(19) United States
(12) Patent Application Publication
(10) Pub. No.: US 2025/0011748 A1
(43) Pub. Date: Jan. 9, 2025

(54) BASE EDITORS, COMPOSITIONS, AND METHODS FOR MODIFYING THE MITOCHONDRIAL GENOME

(71) Applicants: The Broad Institute, Inc., Cambridge, MA (US); University of Washington, Seattle, WA (US); President and Fellows of Harvard College, Cambridge, MA (US)

(72) Inventors: David R. Liu, Cambridge, MA (US); Beverly Mok, Cambridge, MA (US); Joseph D. Mougous, Seattle, WA (US); Snow Brook Peterson, Seattle, WA (US); Marcos de Moraes, Seattle, WA (US); Julian Willis, Cambridge, MA (US)

(73) Assignees: The Broad Institute, Inc., Cambridge, MA (US); University of Washington, Seattle, WA (US); President and Fellows of Harvard College, Cambridge, MA (US)

(21) Appl. No.: 17/795,819

(22) PCT Filed: Jan. 28, 2021
(86) PCT No.: PCT/US2021/015580

§ 371 Date: Jul. 27, 2022

Related U.S. Application Data
(60) Provisional application No. 63/038,741, filed on Jun. 12, 2020.
(60) Provisional application No. 62/967,027, filed on Jan. 28, 2020.

(51) Int. Cl. C12N 9/78; C12N 9/22

(52) U.S. Cl. CPC C12N 9/78; C12N 9/22; C07K 2319/07; C07K 2319/09; C07K 2319/81; C12Y 305/04005

(57) ABSTRACT

The specification provides programmable base editors that are capable of introducing a nucleotide change and/or which could alter or modify the nucleotide sequence at a target site in mitochondrial DNA (mtDNA) with high specificity and efficiency. Moreover, the disclosure provides fusion proteins and compositions comprising a programmable DNA binding protein (e.g., a mitoTALE, a mitoZFP, or a CRISPR/Casp) and double-stranded DNA deaminase that is capable of being delivered to the mitochondria and carrying out precise installation of nucleotide changes in the mtDNA. The fusion proteins and compositions are not limited for use with mtDNA, but also may be used for base editing of any double-stranded target DNA.

### RELATED APPLICATIONS

This application is a national stage filing under 35 U.S.C. § 371 of International PCT Application PCT/US2021/015580, filed Jan. 28, 2021, which claims priority under 35 U.S.C. § 119(e) to U.S. Provisional Application, U.S. Ser. No. 62/967,027, filed on Jan. 28, 2020, and to U.S. Provisional Application, U.S. Ser. No. 63/038,741, filed on Jun. 12, 2020, each of which is incorporated herein by reference.

### GOVERNMENT SUPPORT

This invention was made with government support under grant numbers AI080609, AI142756, HG009490, EB022376, GM122455, GM118062, DK089507, and GM095450 awarded by the National Institutes of Health, and grant number HDTRA1-13-1-0014 awarded by the Department of Defense. The government has certain rights in the invention.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Aug. 23, 2022, is named B119570084US03-SUBSEQ-TNG and is 1,627,926 bytes in size.

### BACKGROUND OF THE INVENTION

Inherited or acquired mutations in mitochondrial DNA (mtDNA) can profoundly impact cell physiology and are associated with a spectrum of human diseases, ranging from rare inborn errors of metabolism,⁴ certain cancers,⁵ age-associated neurodegeneration,⁶ and even the aging process itself.^{7,8} Tools for introducing specific modifications to mtDNA are needed both for modeling diseases and for their therapeutic potential. The development of such tools, however, has been constrained in part by the challenge of transporting RNAs into mitochondria, including guide RNAs required to facilitate nucleic acid modification and/or editing using CRISPR-associated proteins.⁹

Each mammalian cell contains hundreds to thousands of copies of circular mtDNA.¹⁰ Homoplasmy refers to a state in which all mtDNA molecules are identical, while heteroplasmy refers to a state in which a cell contains a mixture of wild-type and mutant mtDNA. Current approaches to engineering and/or altering mtDNA rely on RNA-free DNA-binding proteins, such as transcription activator-like effectors nucleases (mitoTALENs)¹¹⁻¹⁷ and zinc finger nucleases fused to mitochondrial targeting sequences (mitoZFNs), to induce double-strand breaks (DSBs).¹⁸⁻²⁰ Upon cleavage, the linearized mtDNA is rapidly degraded,²¹⁻²³ resulting in heteroplasmic shifts to favor uncut mtDNA genomes. As a candidate therapy however, this approach cannot be applied to homoplasmic mtDNA mutations²⁴ since destroying all mtDNA copies is presumed to be harmful.^{22,25} In addition, using DSBs to eliminate heteroplasmic mtDNA mutations, which tend to be functionally recessive,²⁶ implicitly requires the edited cell to restore its wild-type mtDNA copy number. During this transient period of mtDNA repopulation, the loss of mtDNA copies could cause cellular toxicity resulting in deleterious effects (e.g., apoptosis).

A favorable alternative to targeted destruction of DNA through DSBs is precision genome editing, a capability that has not yet been reported for mtDNA. The ability to precisely install or correct pathogenic mutations, rather than destroy targeted mtDNA, could accelerate our ability to model mtDNA diseases in cells and animal models, and in principle could also enable therapeutic approaches that correct pathogenic mtDNA mutations.

Therefore, the development of programmable base editors that are capable of introducing a nucleotide change and/or which could alter or modify the nucleotide sequence at a target site with high specificity and efficiency within the mtDNA would substantially expand the scope and therapeutic potential of genome editing technologies.

### SUMMARY

The present disclosure relates in part to the inventors' discovery of a double-stranded DNA deaminase, referred to herein as “DddA,” and to its application in base editing of double-stranded nucleic acid molecules, and in particular, the editing of mitochondrial DNA.

Inherited or acquired mutations in mitochondrial DNA (mtDNA) can profoundly impact cell physiology and are associated with a spectrum of human diseases, ranging from rare inborn errors of metabolism,⁴ certain cancers,⁵ age-associated neurodegeneration,⁶ and even the aging process itself.^{7,8} Tools for introducing specific modifications to mtDNA are urgently needed both for modeling diseases and for their therapeutic potential. The present disclosure provides such tools through the use of the newly discovered DddA and variants thereof (e.g., split variants) described herein in base editing of mtDNA, and other double-stranded DNA targets.

Each mammalian cell contains hundreds to thousands of copies of a circular mtDNA¹⁰. Homoplasmy refers to a state in which all mtDNA molecules are identical, while heteroplasmy refers to a state in which a cell contains a mixture of wild-type and mutant mtDNA. Current approaches to engineer mtDNA rely on DNA-binding proteins such as transcription activator-like effectors nucleases (mitoTALENs)¹¹⁻¹⁷ and zinc finger nucleases (mitoZFNs)¹⁸⁻²⁰ fused to mitochondrial targeting sequences to induce double-strand breaks (DSBs). Such proteins do not rely on nucleic acid programmability (e.g., such as with Cas9 domains). Linearized mtDNA is rapidly degraded,²¹⁻²³ resulting in heteroplasmic shifts to favor uncut mtDNA genomes. As a candidate therapy however, this approach cannot be applied to homoplasmic mtDNA mutations²⁴ since destroying all mtDNA copies is presumed to be harmful.^{22,25} In addition, using DSBs to eliminate heteroplasmic mtDNA mutations, which tend to be functionally recessive,²⁶ implicitly requires the edited cell to restore its wild-type mtDNA copy number. During this transient period of mtDNA repopulation, the loss of mtDNA copies could result in cellular toxicity.

As described herein, the disclosure provides a novel platform of precision genome editing using a double-stranded DNA deaminase and a programmable DNA binding protein, such as a TALE domain, zinc finger binding domain, or a napDNAbp (e.g., Cas9), to target the deamination of a target base, which through cellular DNA repair and/or replication, is converted to a new base, thereby installing a base edit at a target site. In some embodiments, the deaminase activity is a cytidine deminase, which deaminates a cytidine, leading to a C-to-T edit at that site. In some other embodiments, that deaminase activity is an adenosine deminase, which deaminates an adenosine, leading to a A-to-G edit at that site. In various embodiments, the disclosure further relates to “split-constructs” and “split-delivery” of said constructs whereby to address the toxic nature of fully active DddA in cells (as discovered by the inventors), the DddA protein is “split” or otherwise divided into two or more DddA fragments which can be separately delivered, expressed, or otherwise provided to cells to avoid the toxicity of fully active DddA. Further, the DddA fragments may be delivered, expressed, or otherwise provided as separate fusion proteins to cells with programmable DNA binding proteins (e.g., zinc finger domains, TALE domains, or Cas9 domains) which are programmed to localize the DddA fragments to a target edit site, through the binding of the DNA binding proteins to DNA sites upstream and downstream of the target edit site. Once co-localized to the target edit site, the separately provided DddA fragments may associate (covalently or non-covalently) to reconstitute an active DddA protein with a double-stranded DNA deaminase activity. In certain embodiments where the objective is to base edit mitochondrial DNA targets, the programmable DNA binding proteins can be modified with one or more mitochondrial localization signals (MLS) so that the DddA-pDNAbp fusions are translocated into the mitochondria, thereby enabling them to act on mtDNA targets.

The inventors are believed to be the first to identify DddA, initially being discovered as a bacterial toxin. The inventors further conceived of the idea of splitting the DddA into two or more domains, which apart do not have a deaminase activity (and as such, lack toxicity), but which may be reconstituted (e.g., inside the cell, and/or inside the mitochondria) to restore the deaminase activity of the protein. This allows the separate delivery DddA fragments to cells (and/or to mitochondria, specifically), or delivery of nucleic acid molecules expressing such DddA fragments to a cell, such that once present or expressed within a cell, DddA fragments may associate with one another. By “associate” it is meant the two or more DddA fragments may come into contact with one another (e.g., in a cell, or within a mitochondria) and form a functional DddA protein within a cell (or mitochondria). The association of the two or more fragments may be through covalent interactions or non-covalent interactions. In addition, the DddA domains may be fused or otherwise non-covalently linked to a programmable DNA binding protein, such as a Cas9 domain or other napDNAbp domain, zinc finger domain or protein (ZF, ZFD, or ZFP), or a transcription activator-like effector protein (TALE), which allows for the co-localization of the two or more DddA fragments to a particular desired site in a target nucleic acid molecule which is to be edited, such that when the DddA fragments are co-localized at the desired editing site, they reform a functional DddA that is capable deaminating a target site on a double-stranded DNA molecule. In certain embodiments, the programmable DNA binding proteins can be engineered to comprise one or more mitochondrial localization signals (MLS) such the DddA domains become translocated into the mitochondria, thereby providing a means by which to conduct base editing directly on the mitochondrial genome.

Accordingly, provided herein are compositions, kits, and methods of modifying double-stranded DNA (e.g., mitochondrial DNA or “mtDNA”) using genome editing strategies that comprise the use of a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in double-stranded DNA (e.g., mtDNA), rather than destroying the DNA (e.g., mtDNA) with double-strand breaks (DSBs). The present disclosure provides pDNAbp polypeptides, DddA polypeptides, fusion proteins comprising pDNAbp polypeptides and DddA polypeptides, nucleic acid molecules encoding the pDNAbp polypeptides, DddA polypeptides, and fusion proteins described herein, expression vectors comprising the nucleic acid molecules described herein, cells comprising the nucleic acid molecules, expression vectors, pDNAbp polypeptides, DddA polypeptides, and/or fusion proteins described herein, pharmaceutical compositions comprising the polypeptides, fusion proteins, nucleic acid molecules, vectors, or cells described herein, and kits comprising the polypeptides, fusion proteins, nucleic acid molecules, vectors, or cells described herein for modifying double-stranded DNA (e.g., mtDNA) by base editing.

In some embodiments, the pDNAbps (e.g., a mitoTALE, mitoZFP, or a CRISPR/Cas) and the DddAs are expressed as fusion proteins. In other embodiments, the pDNAbps and DddAs are expressed as separate polypeptides. In various other embodiments, the fusion proteins and/or the separately expressed pDNAbps and DddAs become translocated into the mitochondria. To effect translocation, the fusion proteins and/or the separately expressed pDNAbps and DddAs can comprise one or more mitochondrial targeting sequences (MTS).

In still other embodiments, the DddA is administered to a cell in which mitochondrial base editing is desired as two or more fragments, wherein each fragment by itself is inactive with respect to deaminase activity, but upon co-localization in the cell, e.g., inside the mitochondria, the two or more fragments reconstitute the deaminase activity.

In certain embodiments, the reconstituted activity of the co-localized two or more fragments can comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 99.9% of the deaminase activity of a wildtype DddA.

In certain embodiments, the DddA is separated into two fragments by dividing the DddA at a split site. A “split site” refers to a position between two adjacent amino acids (in a wildtype DddA amino acid sequence) that marks a point of division of a DddA. In certain embodiments, the DddA can have at least one split site, such that once divided at that split site, the DddA forms an N-terminal fragment and a C-terminal fragment. The N-terminal and C-terminal fragments can be the same or different sizes (or lengths), wherein the size and/or polypeptide length depends on the the location or position of the split site. As used herein, reference to a “fragment” of DddA (or any other polypeptide) can be referred equivalently as a “portion.” Thus, a DddA which is divided at a split site can form an N-terminal portion and a C-terminal portion. Preferably, the N-terminal fragment (or portion) and the C-terminal fragment (or portion) or DddA do not have deaminase activity, or have a reduced deaminase activity that is reduced by at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or up to 100% relative to the wild type DddA activity.

In various embodiments, a DddA may be split into two or more inactive fragments by directly cleaving the DddA at one or more split sites. Direct cleaving can be carried out by a protease (e.g., trypsin) or other enzyme or chemical reagent. In certain embodiments, such chemical cleavage reactions can be designed to be site-selective (e.g., Elashal and Raj, “Site-selective chemical cleavage of peptide bonds,” Chemical Communications, 2016, Vol. 52, pages 6304-6307, the contents of which are incorporated herein by reference.) In other embodiments, chemical cleavage reactions can be designed to be non-selective and/or occur in a random fashion.

In other embodiments, the two or more inactive DddA fragments can be engineered as separately expressed polypeptides. For instance, for a DddA having one split site, the N-terminal DddA fragment could be engineered from a first nucleotide sequence that encodes the N-terminal DddA fragment (which extends from the N-terminus of the DddA up to and including the residue on the amino-terminal side of the split site). In such an example, the C-terminal DddA fragment could be engineered from a second nucleotide sequence that encodes the C-terminal DddA fragment (which extends from the carboxy-terminus of the split site up to including the natural C-terminus of the DddA protein). The first and second nucleotide sequences could be on the same or different nucleotide molecules (e.g., the same or different expression vectors).

In various embodiments, the N-terminal portion of the DddA may be referred to as “DddA-N half” and the C-terminal portion of the DddA may be referred to as the “DddA-C half.” Reference to the term “half” does not connote the requirement that the DddA-N and DddA-C portions are identically half of the size and/or sequence length of a complete DddA, or that the split site is required to be at the mid point of the complete DddA polypeptide. To the contrary, and as noted above, the split site can be between any pair of residues in the DddA polypeptide, thereby giving rise to half portions which are unequal in size and/or sequence length. In certain embodiments, the split site is within a loop region of the DddA.

Accordingly, in one aspect, the disclosure relates to a pair of fusion proteins useful for making modifications to the sequence of mitochondrial DNA (e.g., mtDNA). The pair of fusion proteins, in some embodiments, can comprise a first fusion protein comprising a first pDNAbp (e.g., a mitoTALE, mitoZFP, or a CRISPR/Cas9) and a first portion or fragment of a DddA, and a second fusion protein comprising a second pDNAbp (e.g., mitoTALE, mitoZFP, or a CRISPR/Cas9) and a second portion or fragment of a DddA, such that the first and the second portions of the DddA reconstitute a DddA upon co-localization in a cell and/or mitochondria. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[pDNAbp]-[DddA half^{A}] and [pDNAbp]-[DddA half^{B}];
[DddA-half^{A}]-[pDNAbp] and [DddA-half^{B}]-[pDNAbp];
[pDNAbp]-[DddA half^{A}] and [DddA-half^{B}]-[pDNAbp]; or
[DddA-half^{A}]-[pDNAbp] and [pDNAbp]-[DddA half^{B}], wherein “A” or “B” can be the N-terminal or C-terminal half of DddA.

In another aspect, the disclosure relates to a pair of fusion proteins useful for making modifications to the sequence of mitochondrial DNA (e.g., mtDNA). The pair of fusion proteins can comprise a first fusion protein comprising a first mitoTALE and a first portion or fragment of a DddA, and a second fusion protein comprising a second mitoTALE and a second portion or fragment of a DddA, such that the first and the second portions of the DddA, upon co-localization in a cell and/or mitochondria, are reconstituted as an active DddA. In certain embodiments, that first portion of the DddA is an N-terminal fragment of a DddA and the second portion of the DddA is C-terminal fragment of a Ddda. In other embodiments, the first portion of the DddA is a C-terminal fragment of a DddA and the second portion of the DddA is an N-terminal fragment of a DddA. In this aspect, the structure of the pair of fusion proteins can be, for example:
[mitoTALE]-[DddA half^{A}] and [mitoTALE]-[DddA half^{B}];
[DddA-half^{A}]-[pDNAbp] and [DddA-half^{B}]-[mitoTALE];
[mitoTALE]-[DddA half^{A}] and [DddA-half^{B}]-[mitoTALE]; or
[DddA-half^{A}]-[mitoTALE] and [mitoTALE]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first mitoZFP and a first portion or fragment of a DddA, and a second fusion protein comprising a second mitoZFP and a second portion or fragment of a DddA, such that the first and the second portions of the DddA, upon co-localization in a cell and/or mitochondria, are reconstituted as an active DddA. In certain embodiments, that first portion of the DddA is an N-terminal fragment of a DddA and the second portion of the DddA is C-terminal fragment of a Ddda. In other embodiments, the first portion of the DddA is a C-terminal fragment of a DddA and the second portion of the DddA is an N-terminal fragment of a DddA. In this aspect, the structure of the pair of fusion proteins can be, for example:
[mitoZFP]-[DddA half^{A}] and [mitoZFP]-[DddA half^{B}];
[DddA-half^{A}]-[pDNAbp] and [DddA-half^{B}]-[mitoZFP];
[mitoZFP]-[DddA half^{A}] and [DddA-half^{B}]-[mitoZFP]; or
[DddA-half^{A}]-[mitoZFP] and [mitoZFP]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first Cas9 domain and a first portion or fragment of a DddA, and a second fusion protein comprising a second Cas9 domain and a second portion or fragment of a DddA, such that the first and the second portions of the DddA, upon co-localization in a cell and/or mitochondria, are reconstituted as an active DddA. In certain embodiments, that first portion of the DddA is an N-terminal fragment of a DddA (i.e., “DddA half^{A}” as shown in FIGS. 1A-1E) and the second portion of the DddA is C-terminal fragment of a DddA (i.e., “DddA half^{B}” as shown in FIGS. 1A-1E). In other embodiments, the first portion of the DddA is an C-terminal fragment of a DddA and the second portion of the DddA is an N-terminal fragment of a DddA. In this aspect, the structure of the pair of fusion proteins can be, for example:
[Cas9]-[DddA half^{A}] and [Cas9]-[DddA half^{B}];
[DddA-half^{A}]-[Cas9] and [DddA-half^{B}]-[Cas9];
[Cas9]-[DddA half^{A}] and [DddA-half^{B}]-[Cas9]; or
[DddA-half^{A}]-[Cas9] and [Cas9]-[DddA half^{B}], wherein “A” or “B” can be the N-terminal or C-terminal half of DddA.

In each instance above of “]-[” can be in reference to a linker sequence.

In some embodiments, a first fusion protein comprises, a first mitochondrial transcription activator-like effector (mitoTALE) domain and a first portion of a DNA deaminase effector (DddA). In some embodiments, the first portion of the DddA comprises an N-terminal truncated DddA. In some embodiments, the first mitoTALE domain is configured to bind a first nucleic acid sequence proximal to a target nucleotide. In some embodiments, the first portion of a DddA is linked to the remainder of the first fusion protein by the C-terminus of the first portion of a DddA.

In some embodiments, a second fusion protein comprises, a second mitoTALE domain and a second portion of a DddA. In some embodiments, the second portion of the DddA comprises a C-terminal truncated DddA. In some embodiments, the second mitoTALE domain is configured to bind a second nucleic acid sequence proximal to a nucleotide opposite the target nucleotide. In some embodiments, the second portion of a DddA is linked to the remainder of the second fusion protein by the C-terminus of the second portion of a DddA.

In some embodiments, the first or second fusion protein is the result of truncations of a DddA at a residue site selected from the group comprising: 62, 71, 73, 84, 94, 108, 110, 122, 135, 138, 148, and 155. In some embodiments, the first or second fusion protein is the result of truncations of a DddA at a residue 148.

In some embodiments, the first or second fusion protein further comprises a linker. In some embodiments, the linker is positioned between the first mitoTALE and the first portion of a DddA and/or between the second mitoTALE and the second portion of a DddA. In some embodiments, the linker is at least two amino acids and no greater than sixteen amino acid residues in length. In some embodiments, the linker is two amino acid residues.

In some embodiments, the first or second fusion protein further comprises at least one uracil glycosylase inhibitor. In some embodiments, the first or second fusion protein the at least one glycosylase inhibitor is attached to the C-terminus of the first and/or second portion of a DddA.

In another aspect, the disclosure relates to a pair of fusion proteins comprising: (a) a first fusion protein disclosed herein; and (b) a second fusion protein disclosed herein, wherein the first pDNAbp (e.g., mitoTALE, mitoZFP, or mitoCas9) of the first fusion protein is configured to bind a first nucleic acid sequence proximal to a target nucleotide and the second pDNAbp (e.g., mitoTALE, mitoZFP, or mitoCas9) of the second fusion protein is configured to bind a second nucleic acid sequence proximal to a nucleotide opposite the target nucleotide. In some embodiments, the first nucleic acid sequence of the pair of fusion proteins is upstream of the target nucleotide and the second nucleic acid of the pair of fusion proteins is upstream of a nucleic acid of the complementary nucleotide.

In another aspect the disclosure relates to a pair of fusion proteins, wherein the first and second fusion proteins disclosed herein, are configured to form a dimer, and dimerization of the first and second fusion proteins at closely spaced nucleic acid sequences reconstitutes at least partial activity of a full length DddA. In some embodiments, the dimerization of the pair of fusion proteins facilitates deamination of the target nucleotide.

In another aspect, the disclosure relates to a recombinant vector comprising an isolated nucleic acid as disclosed herein.

In some embodiments, the vector is part of a composition, the composition comprising the vector and a pharmaceutically acceptable excipient.

In another aspect, the disclosure relates to an isolated cell comprising a nucleic acid as disclosed. In some embodiments, the isolated cell is a mammalian cell. In some embodiments, the mammalian cell is a human cell.

In another aspect, the disclosure relates to a method of treating a subject having, at risk of having, or suspected of having, a disorder comprising administering an effective amount of a pair of fusion proteins as described herein, a nucleic acid as described herein, a vector as disclosed herein, a composition as described herein, and/or an isolated cell as described herein. For example, the disorder can be a mitochondrial disorder, such as, MELAS/Leigh syndrome or Leber's hereditary optic neuropathy.

In another aspect, the disclosure relates to a method of editing a nucleic acid in a subject, comprising: (a) determining a target nucleotide to be deaminated; (b) configuring the first fusion protein to bind proximally to the target nucleotide; (c) configuring a second fusion protein to bind proximally to a nucleotide opposite to the target nucleotide; and (d) administering an effective amount of the first and second fusion proteins, wherein, the first mitoTALE binds proximally to the target nucleotide and the second mitoTALE binds proximally to the nucleotide opposite the target nucleotide, and wherein the first portion of a DddA dimerizes with the second portion of a DddA, wherein the dimer has at least some activity native to full length DddA, and wherein the activity deaminates the target nucleotide.

In some embodiments, the disorder treated by the methods described herein is a genetic disorder. In some embodiments, the genetic disorder is a mitochondrial genetic disorder. In some embodiments, the mitochondrial disorder is selected from: MELAS/Leigh syndrome and Leber's hereditary optic neuropathy. In some embodiments, the mitochondrial disorder is MELAS/Leigh syndrome. In some embodiments, the mitochondrial disorder is Leber's hereditary optic neuropathy.

In some embodiments, the subject treated by the methods described herein is a mammal. In some embodiments, the mammal is human.

In another aspect, the disclosure relates to a kit comprising the first and/or second fusion proteins as disclosed herein, the pair of fusion proteins as disclosed herein, the dimer as disclosed herein, the nucleic acids as disclosed herein, the vector as disclosed herein, the composition as disclosed herein, and/or the isolated cell as disclosed herein. The vector may be an AAV vector (e.g., AAV1, AAV2, AAV3, AAV4, AAV5, AAV6, AAV7, AAV8, AAV9, or other serotype), a lentivirus vector, and may include one or more promoters that regulate the expression of the nucleotide sequences encoding the pair of fusion proteins.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

All previously described cytidine deaminases, including those used in base editing, operate on single-stranded DNA and thus when used for genome editing require unwinding of double-stranded DNA by macromolecules such as CRISPR-Cas9 complexed with a guide RNA. The difficulty of delivering guide RNAs into the mitochondria has thus far precluded base editing in mitochondrial DNA (mtDNA). The ability of DddA to deaminate double-stranded DNA raises the possibility of RNA-free precision base editing, rather than simple elimination of targeted mtDNA copies following double-strand DNA breaks. Split-DddA halves were engineered that are non-toxic and inactive until brought together on target DNA by adjacently bound programmable DNA-binding proteins. Fusions of the split-DddA halves, TALE array proteins, and uracil glycosylase inhibitor resulted in RNA-free DddA-derived cytosine base editors (DdCBEs) that catalyze C·G-to-T·A conversions efficiently and with high DNA sequence specificity and product purity at targeted sites within mtDNA in human cells.

DddA-mediated base editing was used to model a disease-associated mtDNA mutation in human cell lines, resulting in changes in rates of respiration and oxidative phosphorylation. CRISPR-free, DddA-mediated base editing enables precision editing of mtDNA, with important basic science and biomedical implications.

FIG. 1A is a schematic representation of a naturally occurring interbacterial toxin discovered by the inventors and catalyzes unprecedented deamination of cytidines within double-stranded DNA as a substrate. The protein is referred to as a double-stranded DNA deaminase, which is referred to herein as a “DddA.” The inventors are believed to be the first to identify such a deaminase. However, in its naturally occurring form, the inventors discovered that DddA is toxic to cells. The inventors have conceived of the idea of using the DddA in the context of base editing to deaminate a nucleobase at a target edit site.

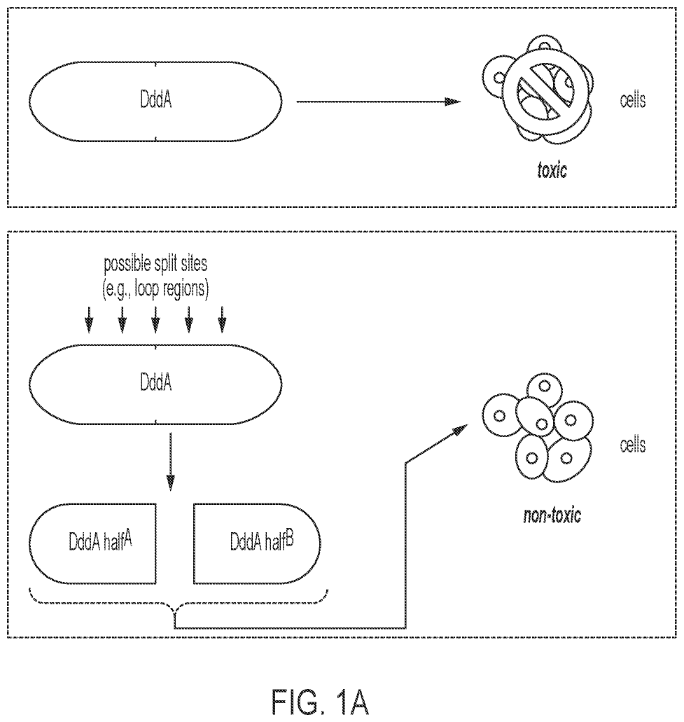

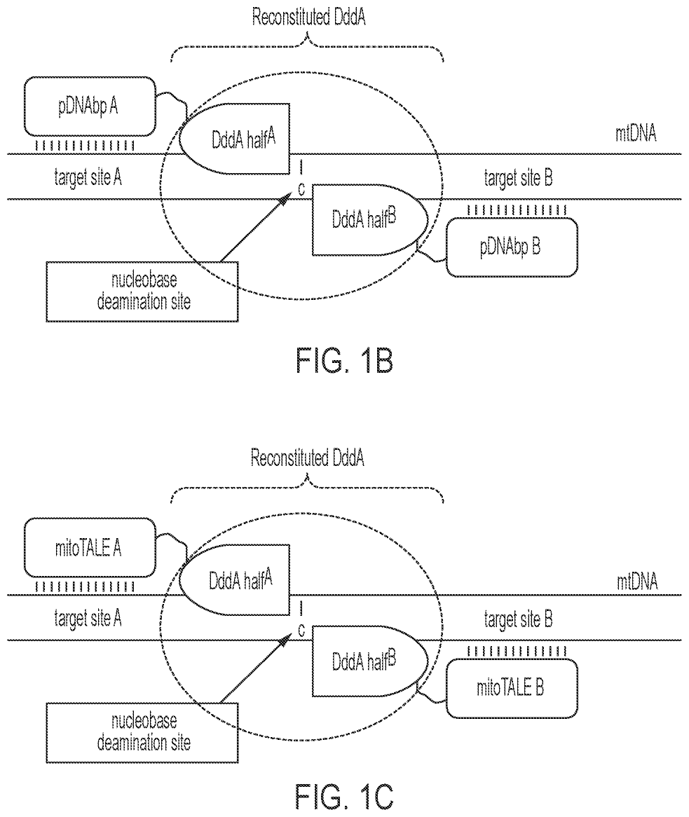

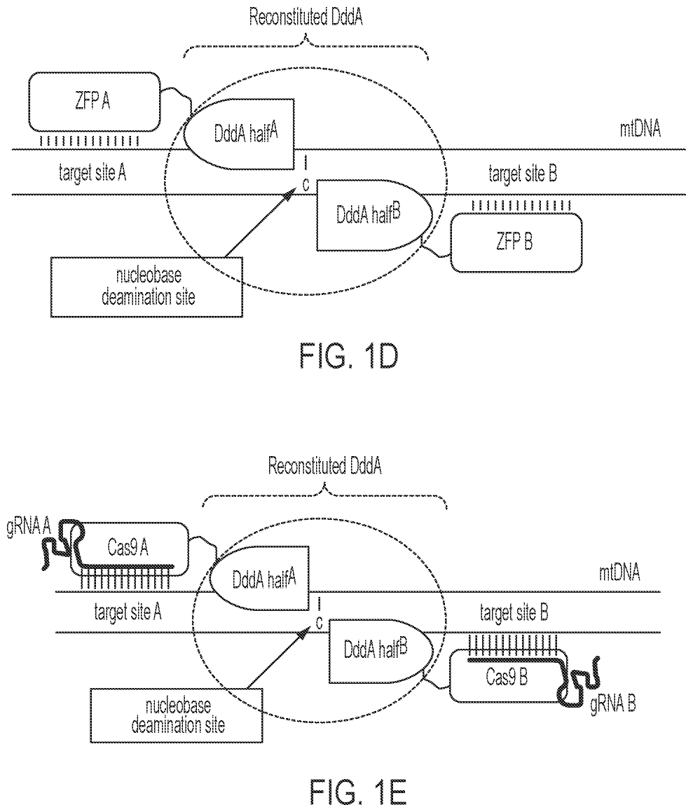

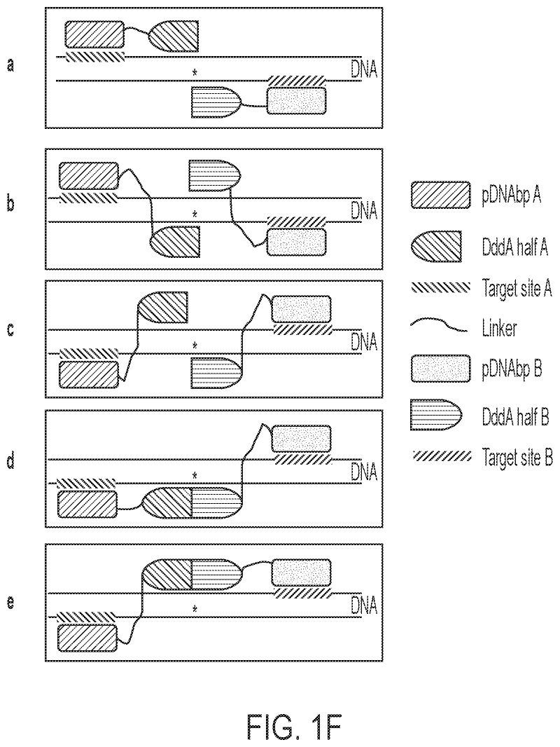

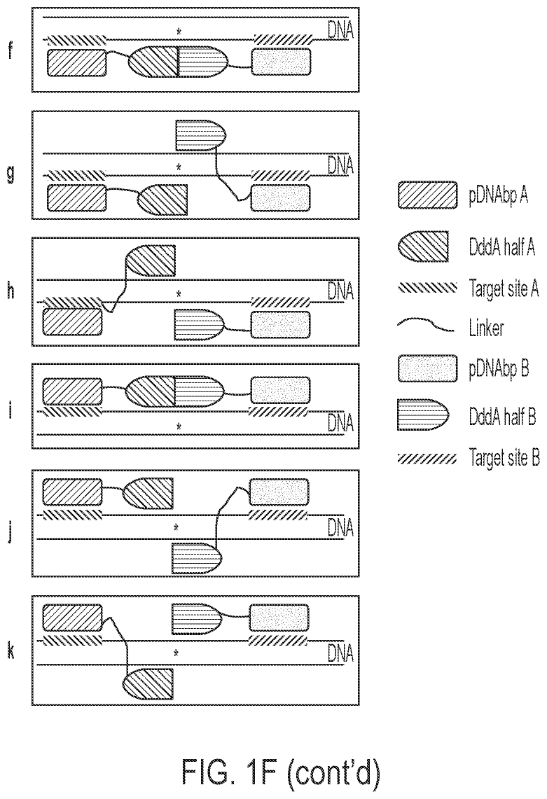

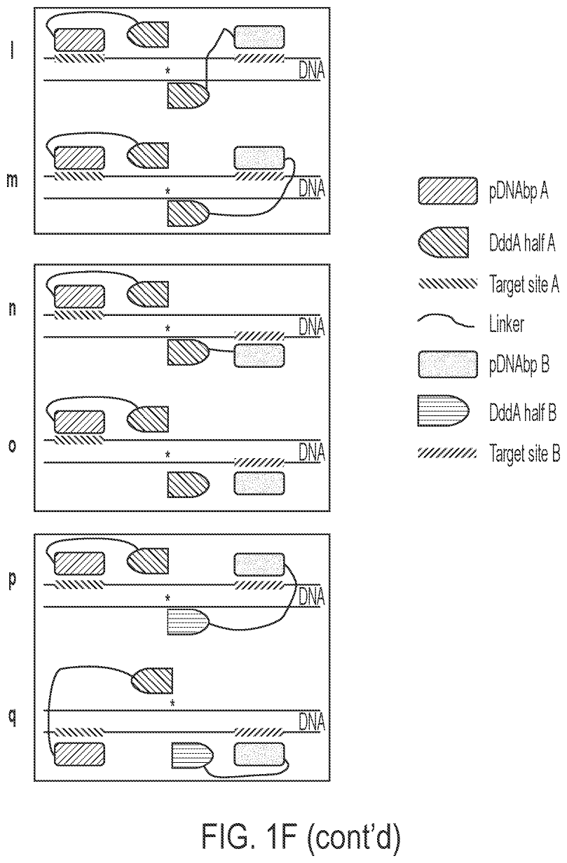

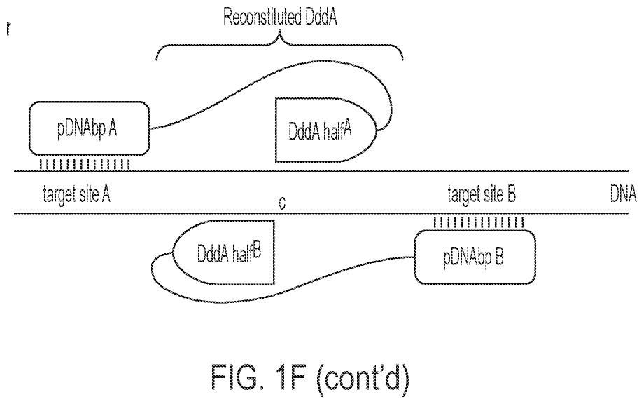

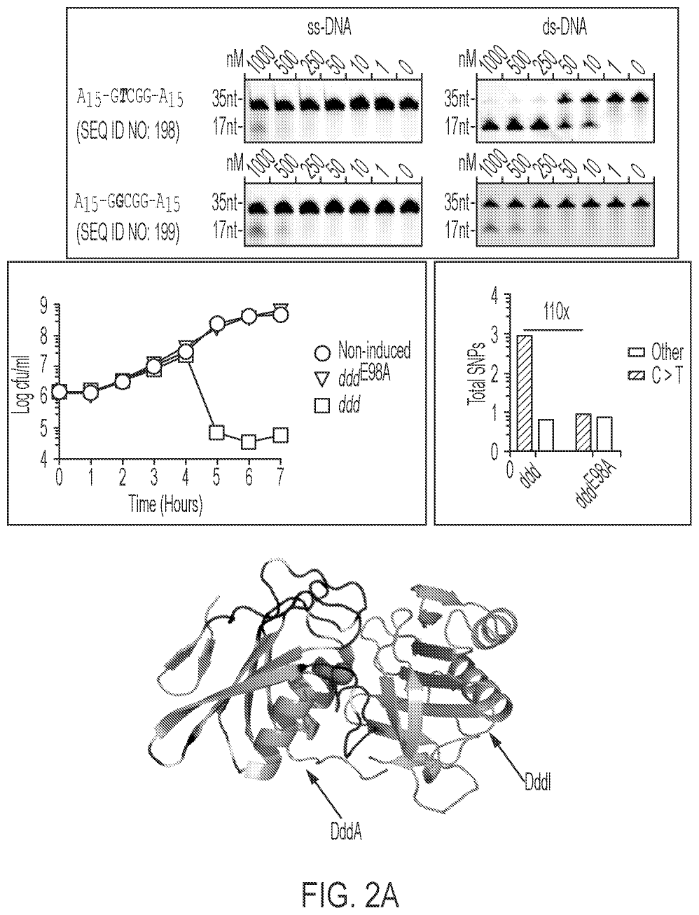

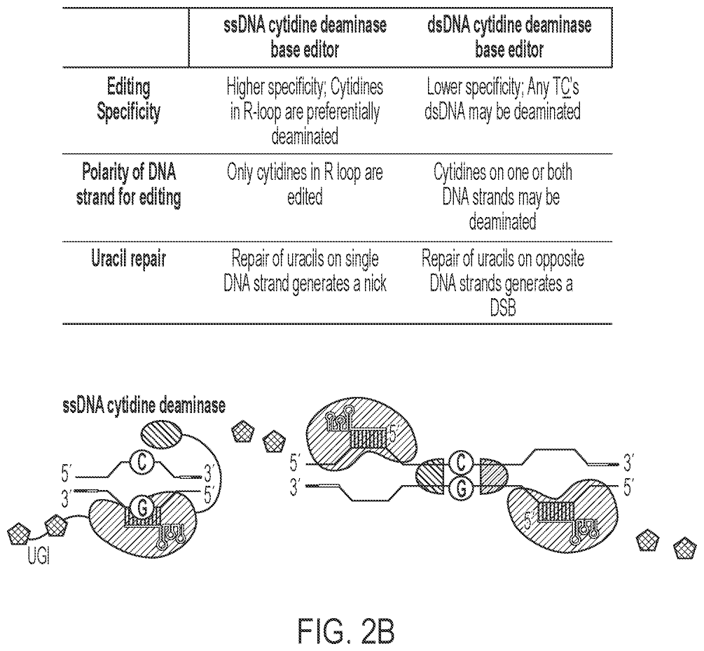

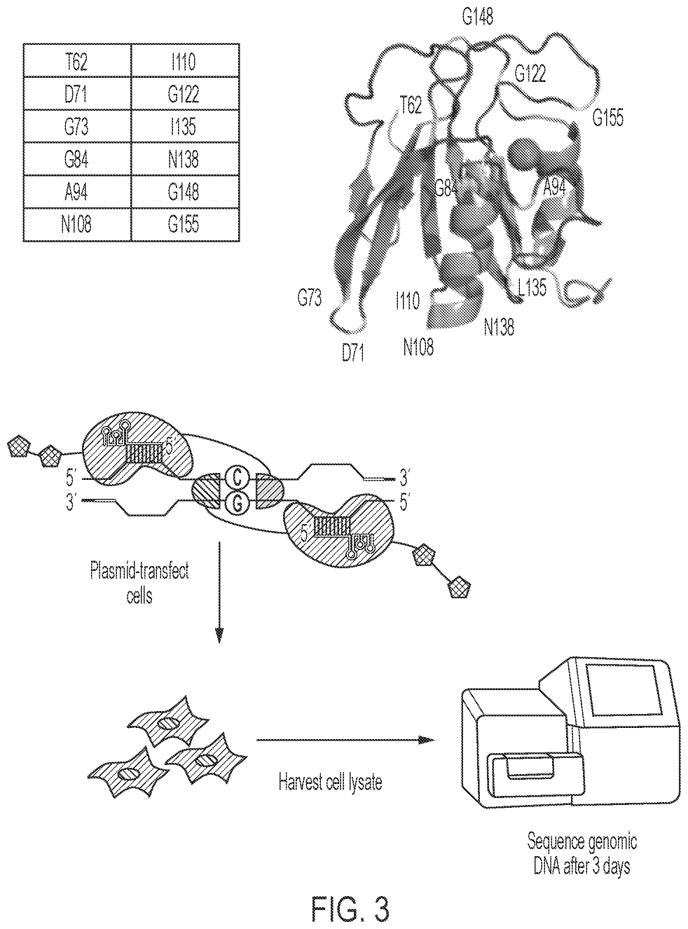

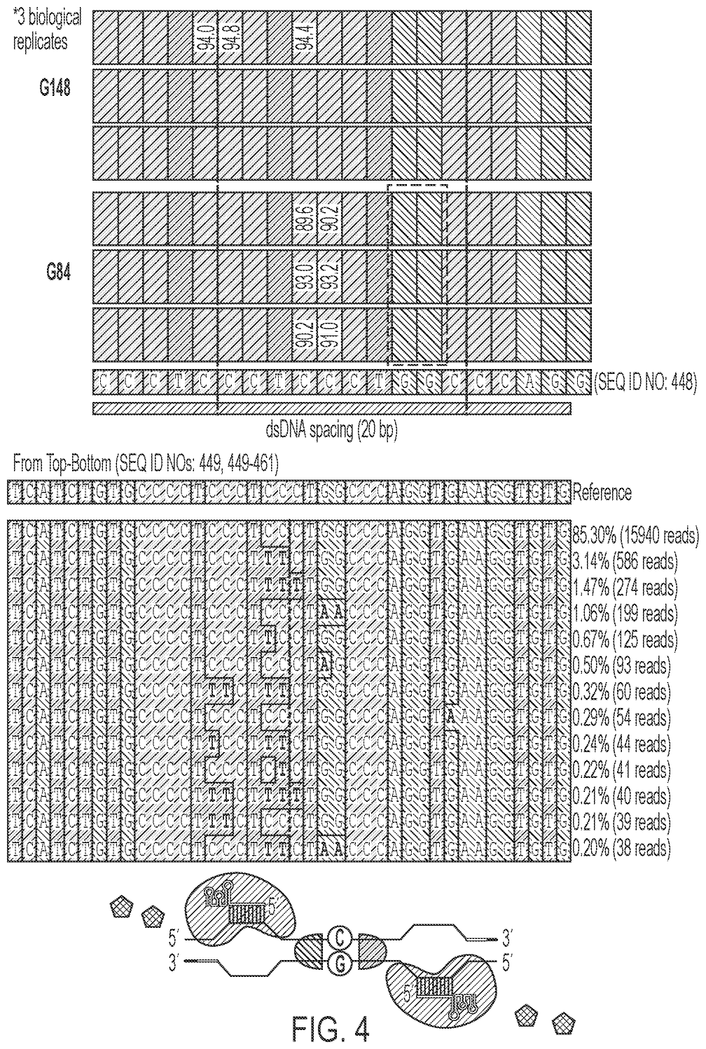

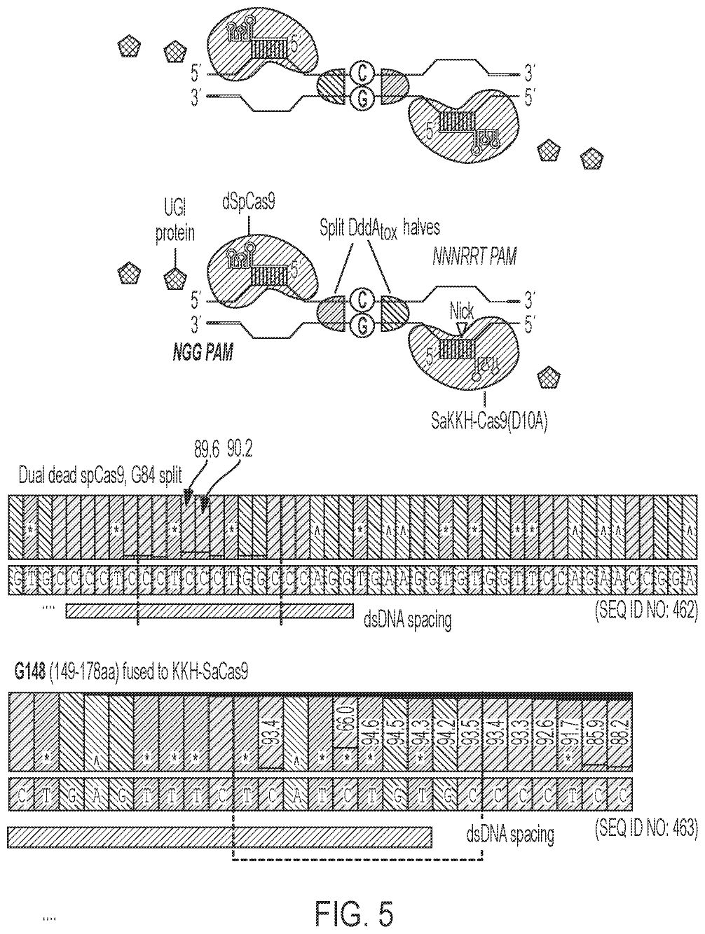

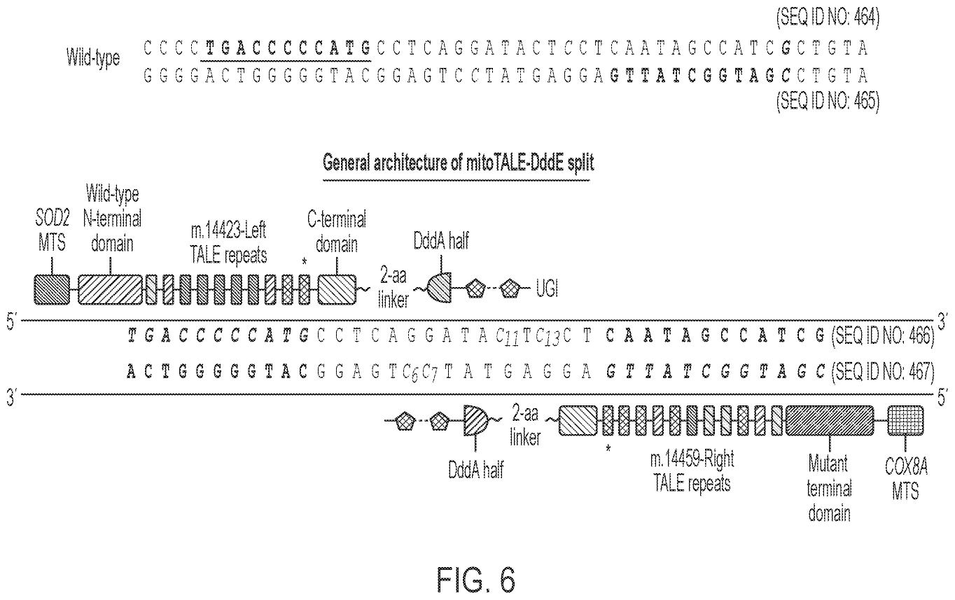

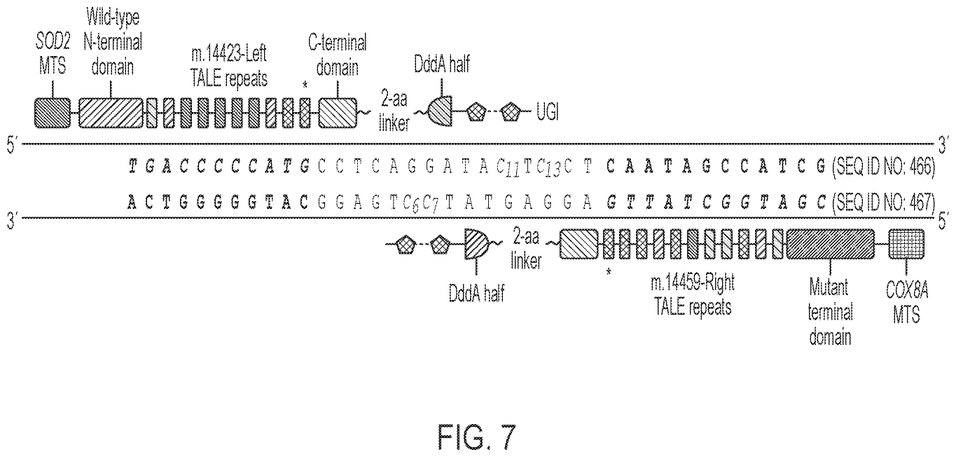

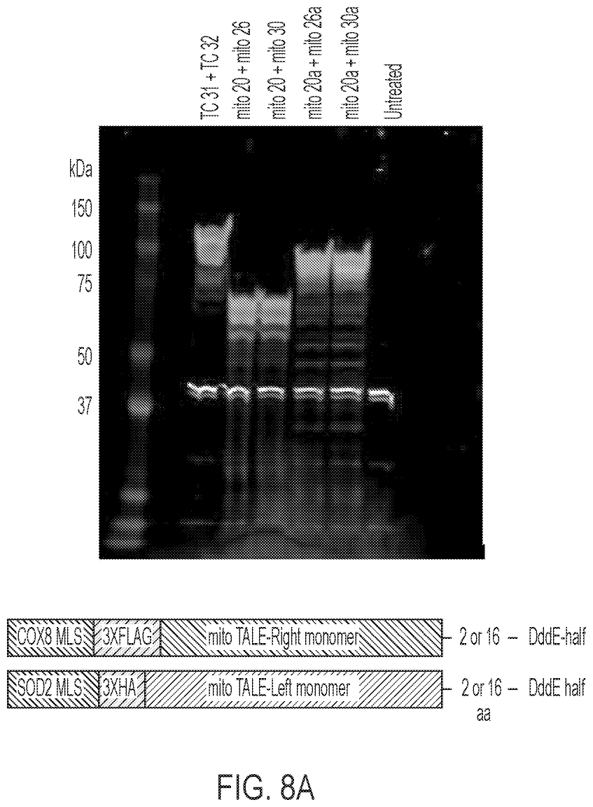

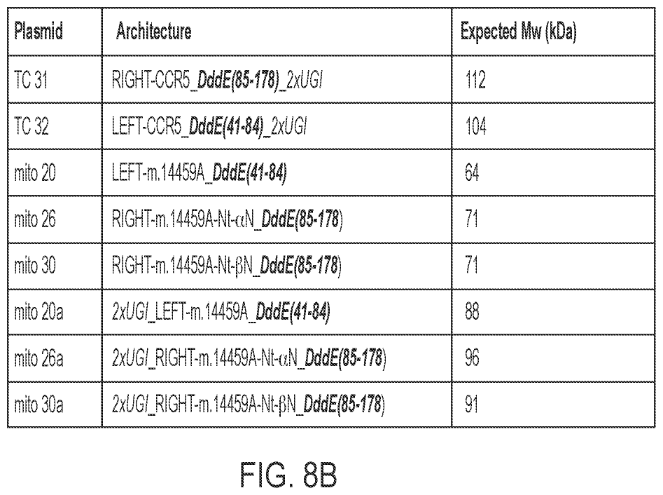

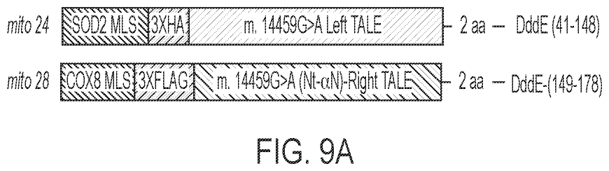

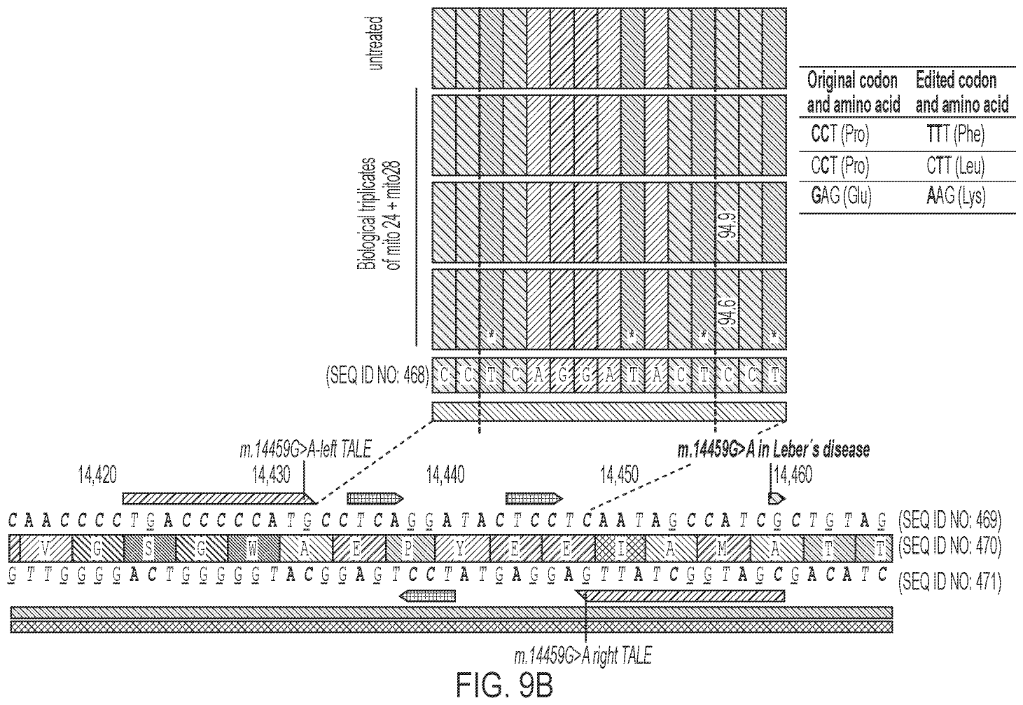

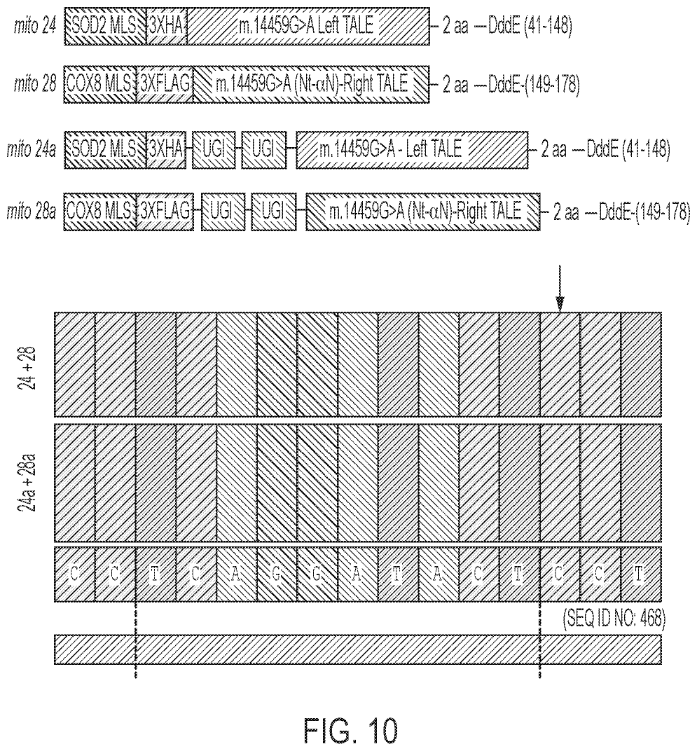

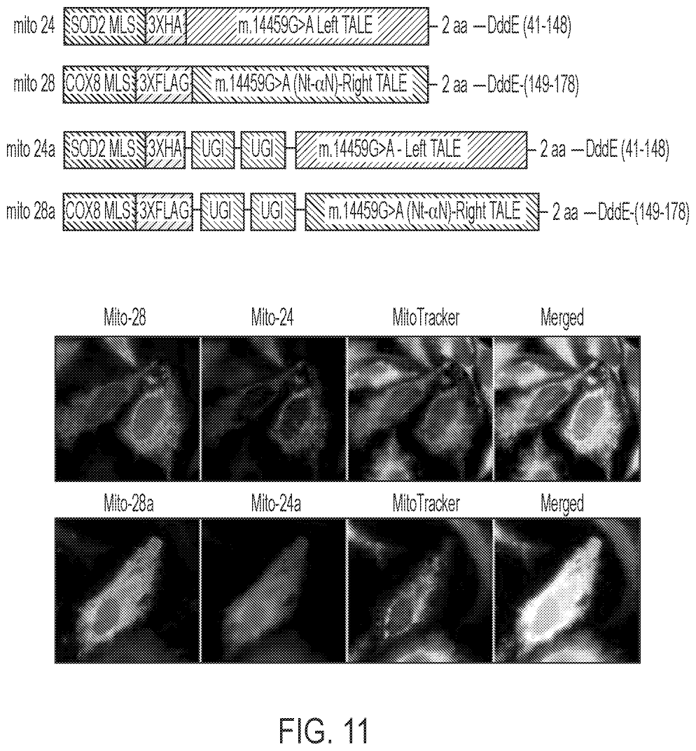

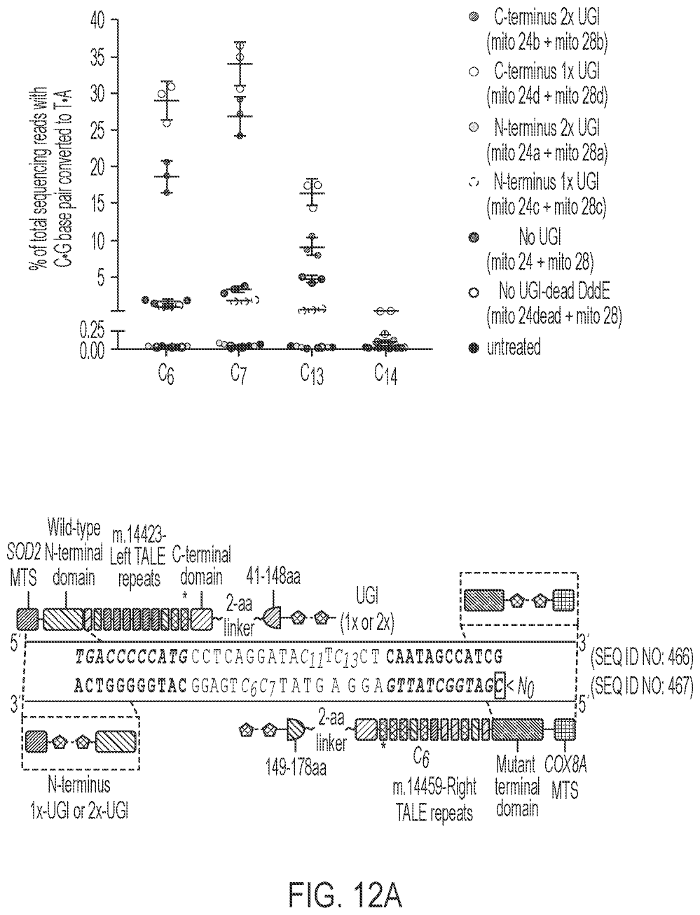

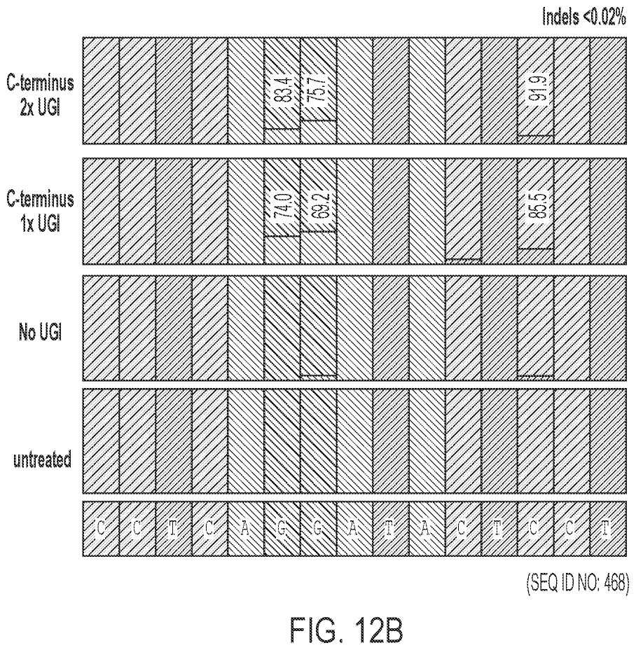

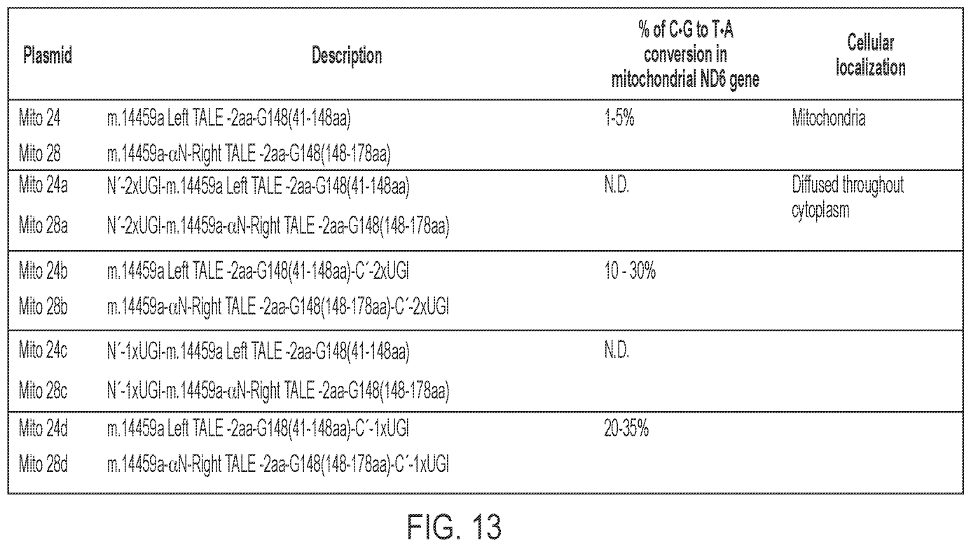

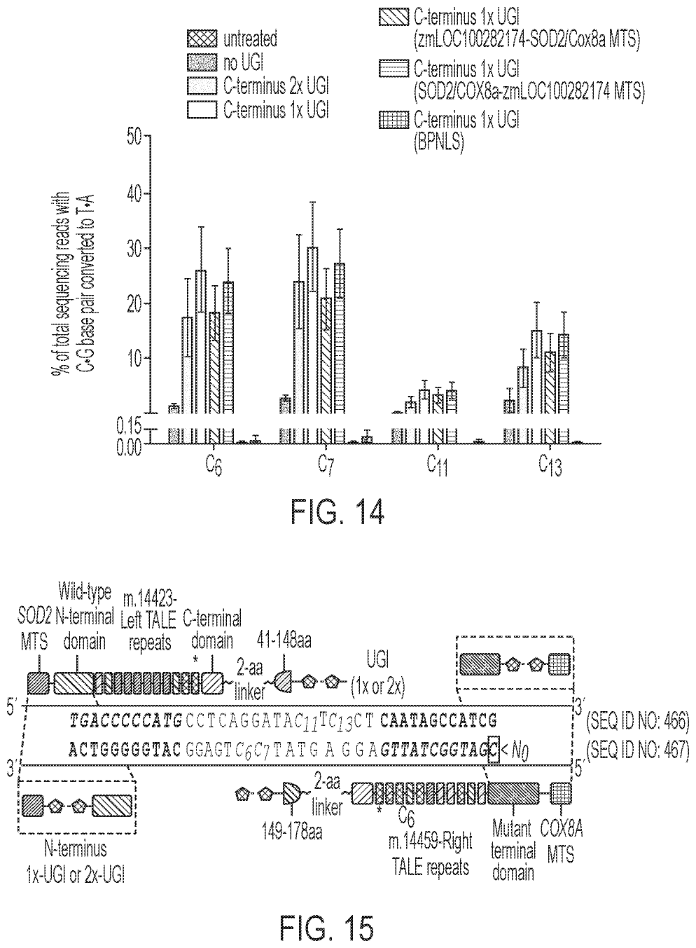

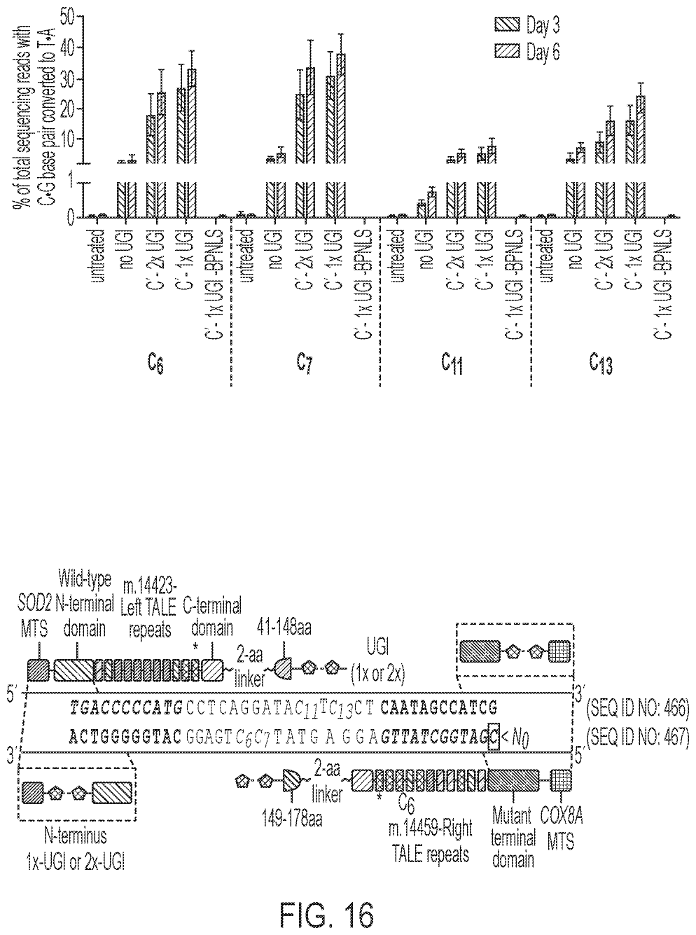

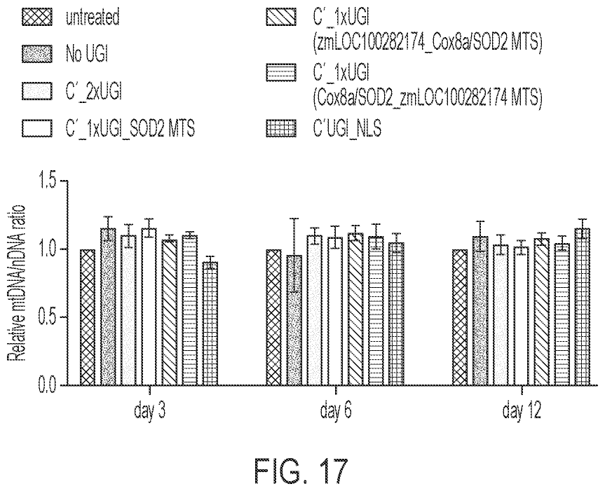

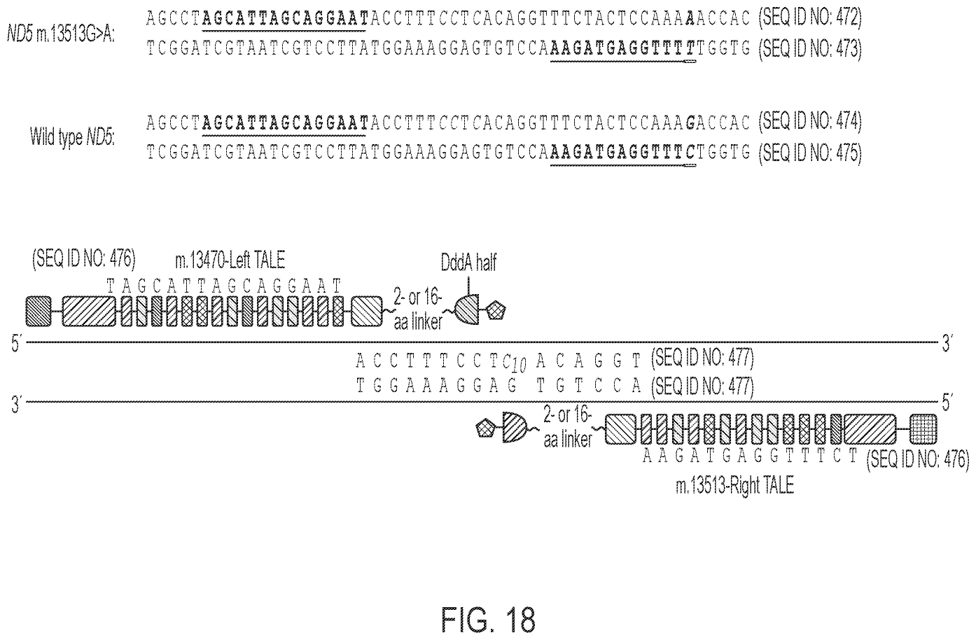

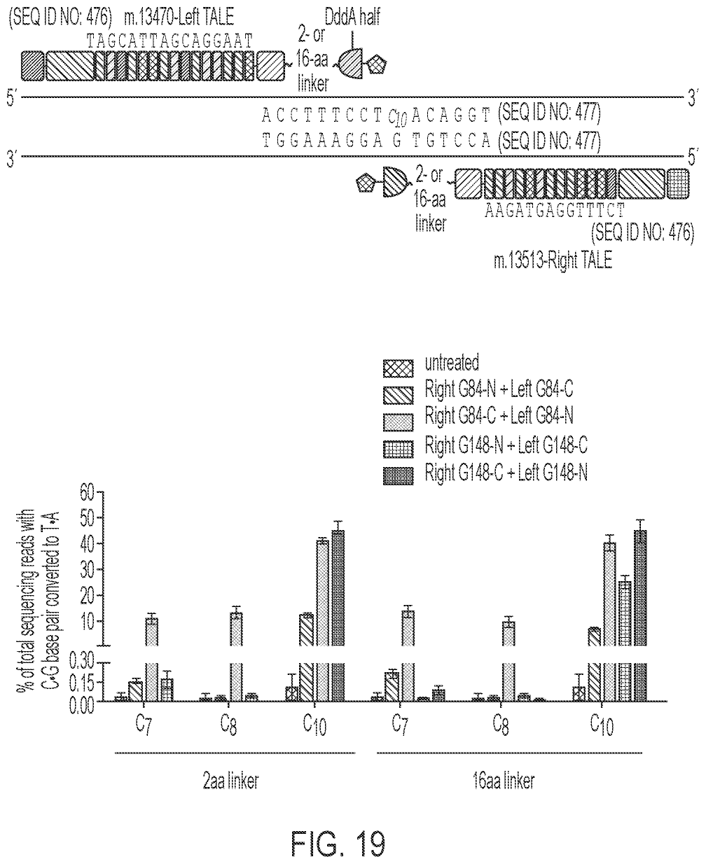

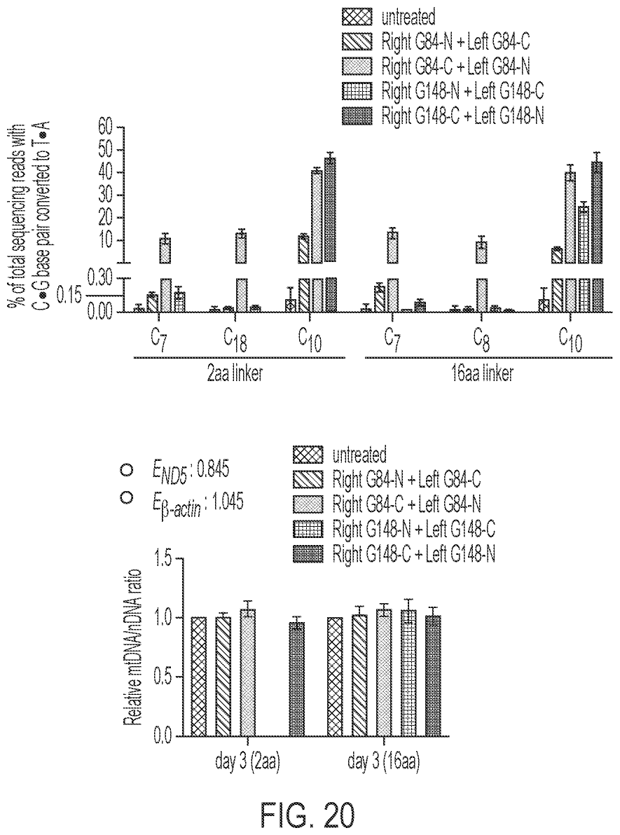

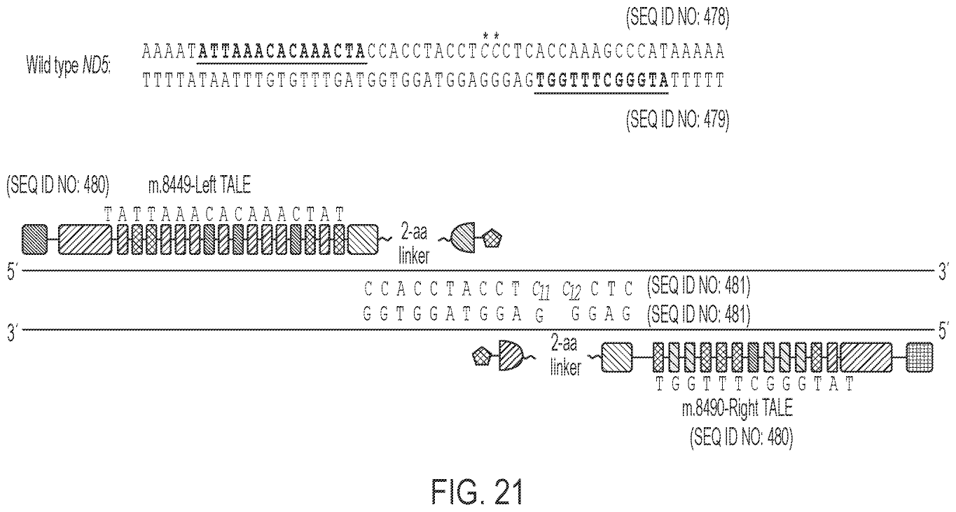

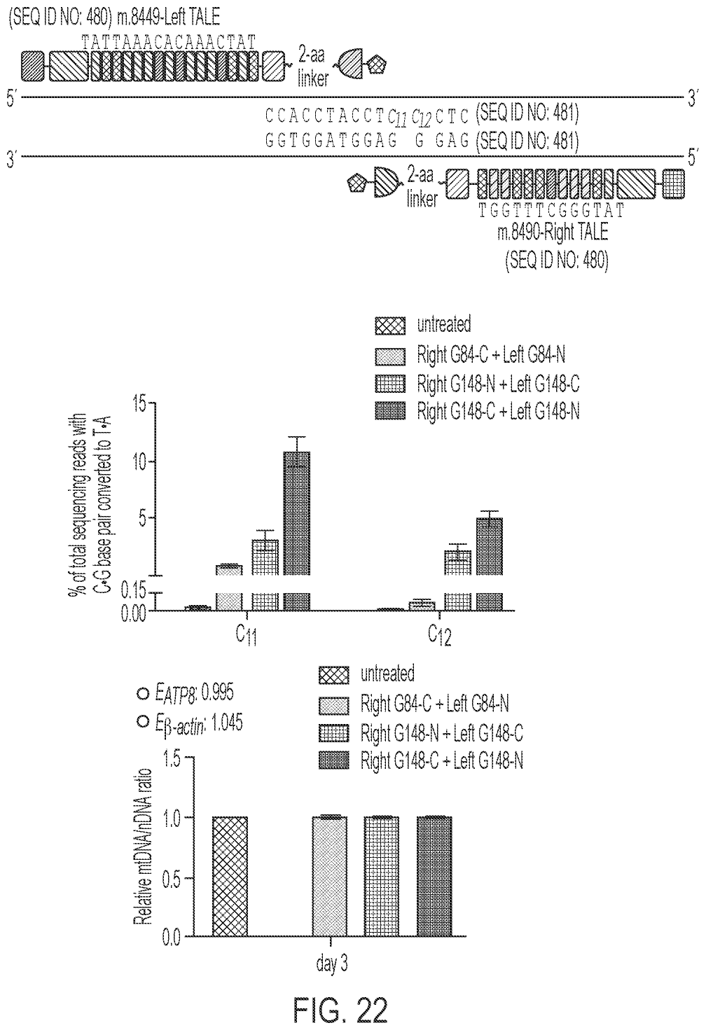

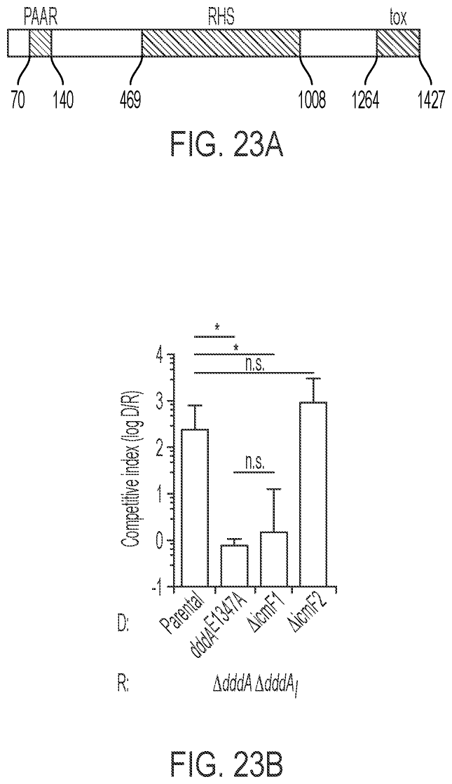

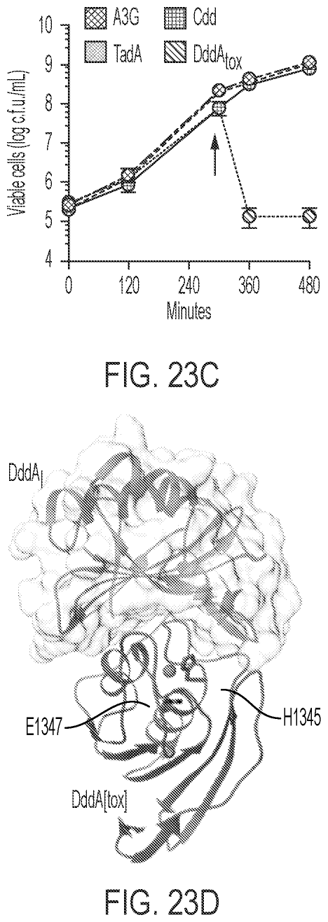

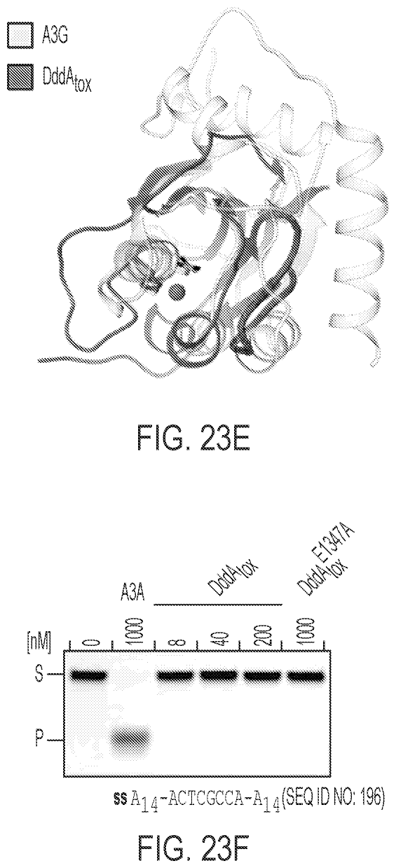

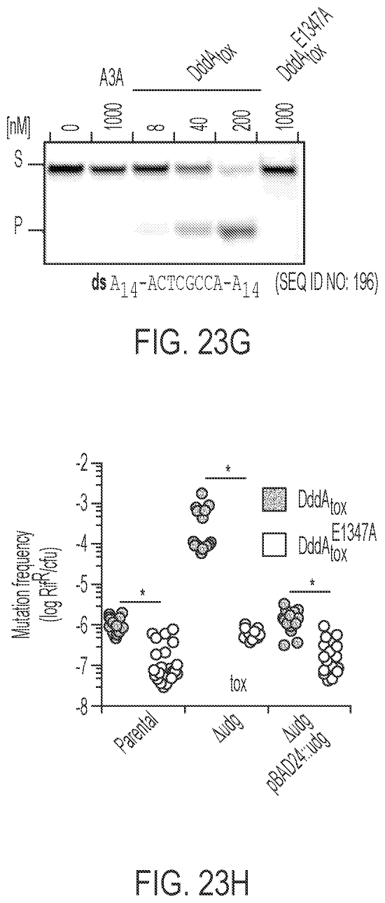

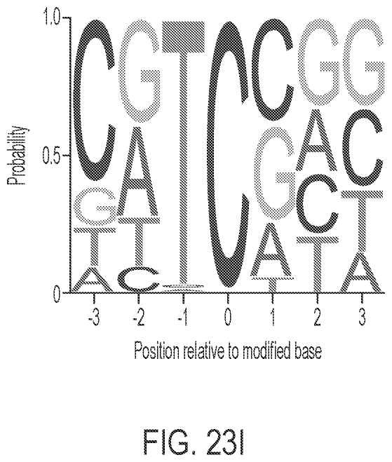

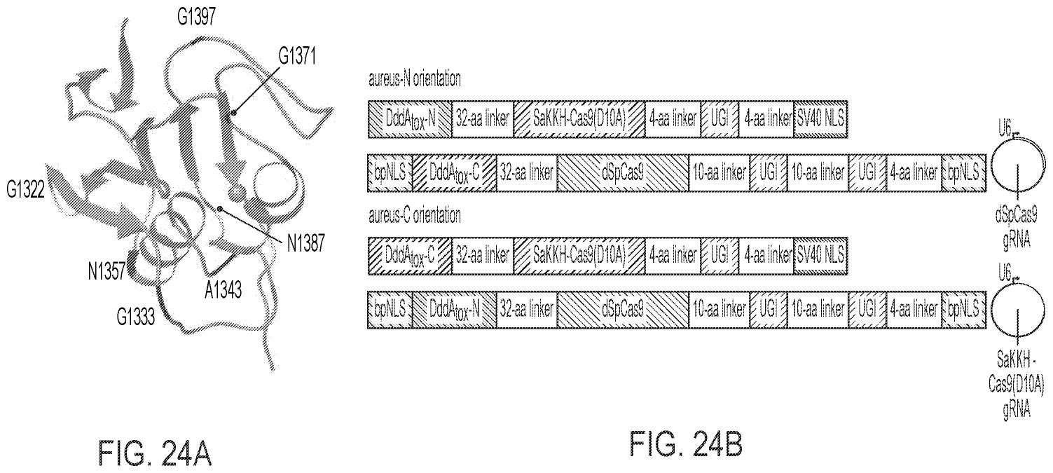

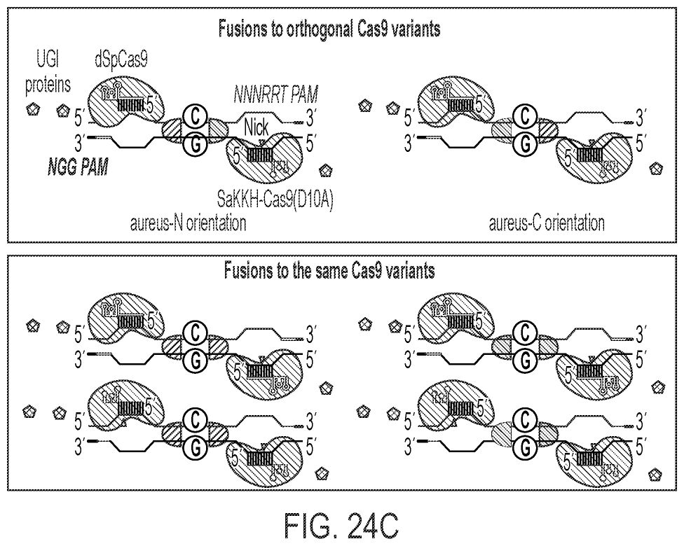

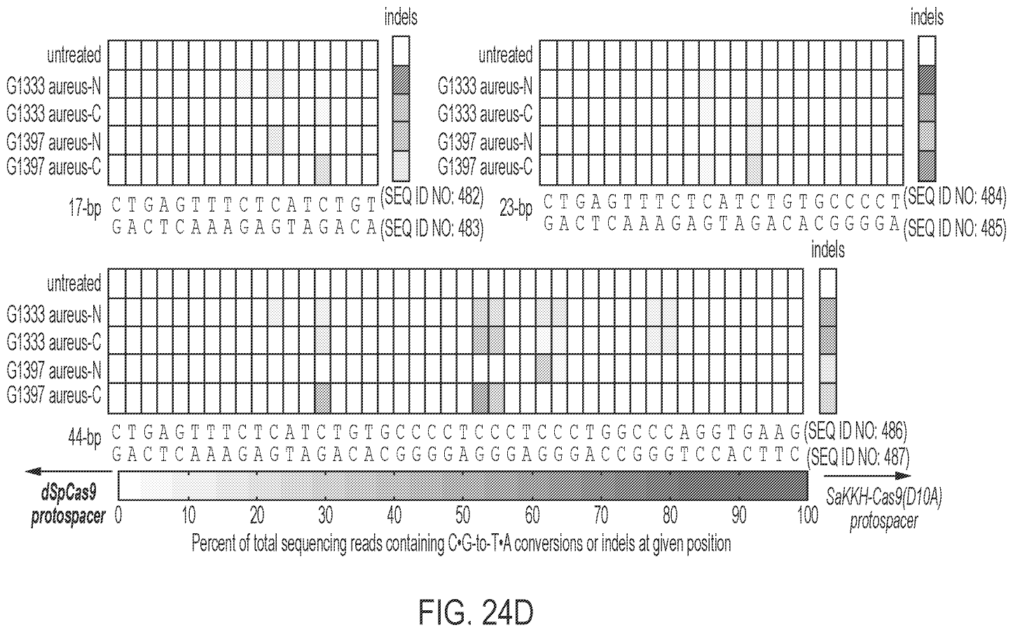

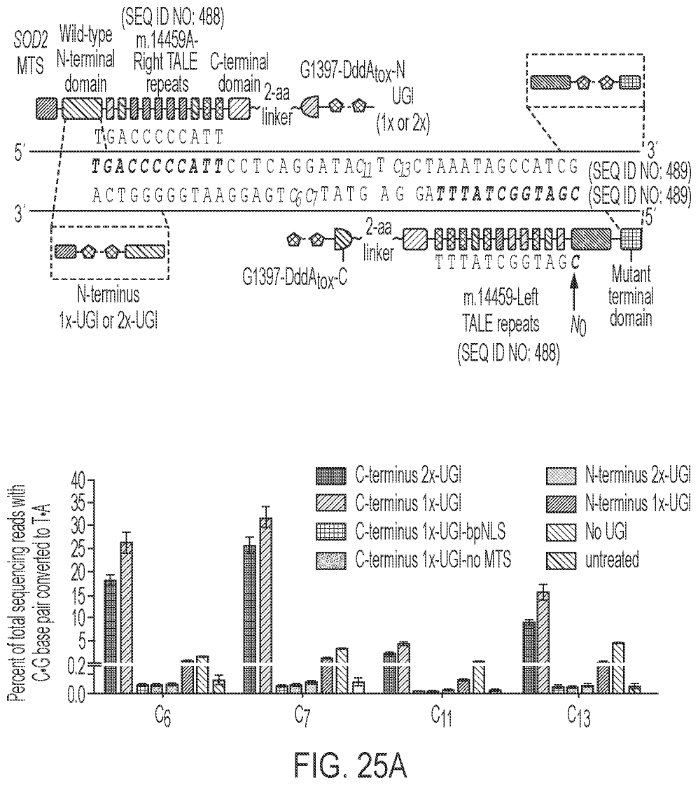

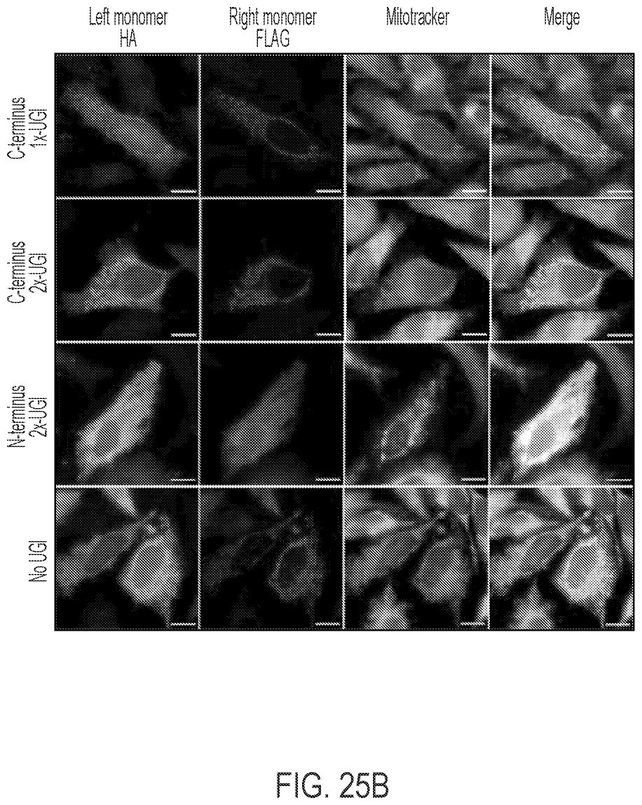

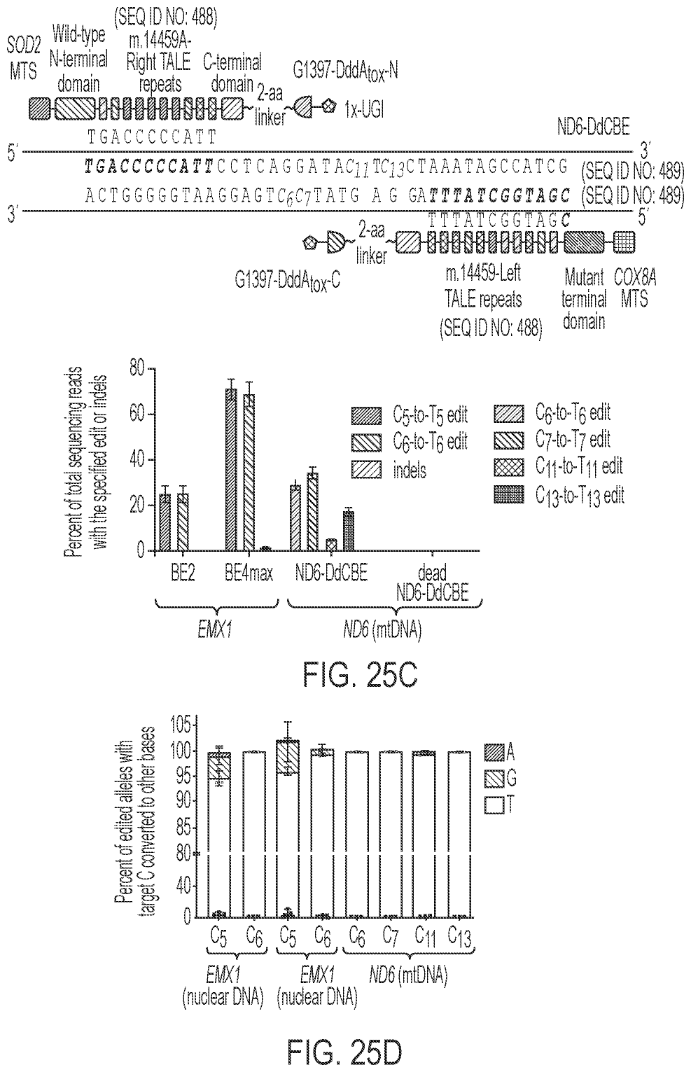

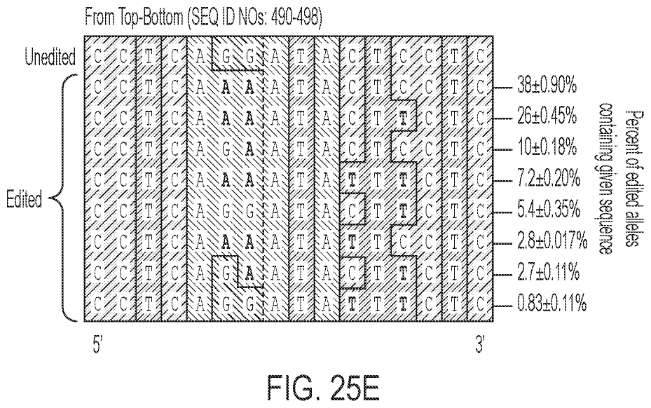

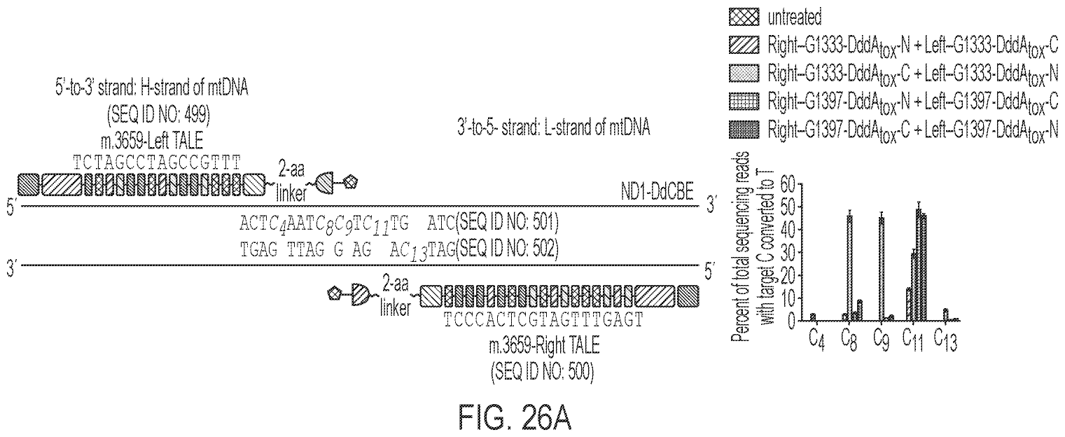

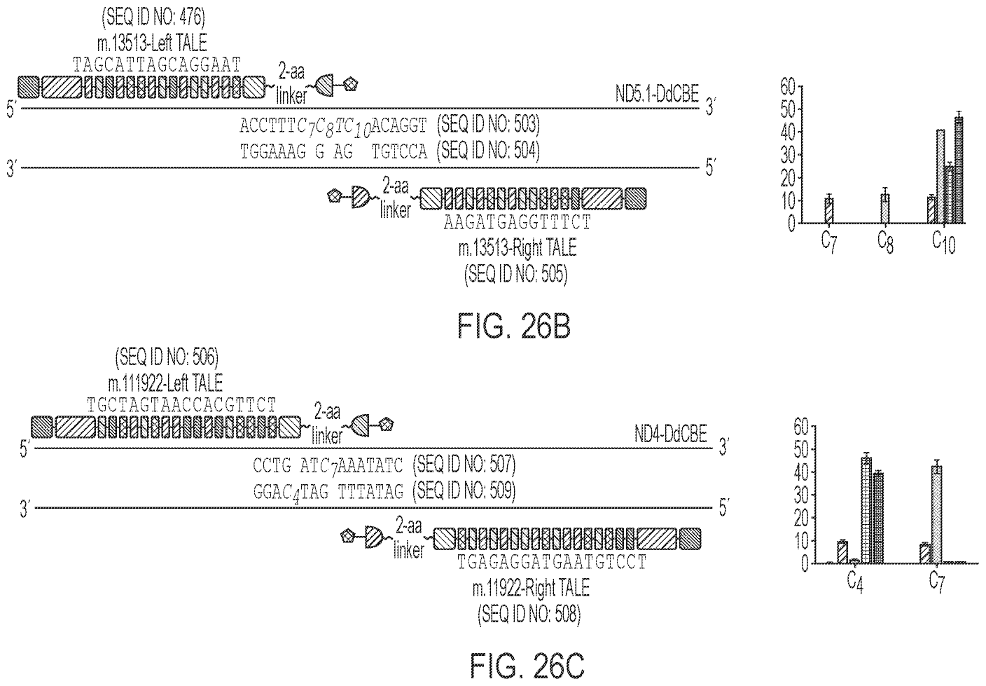

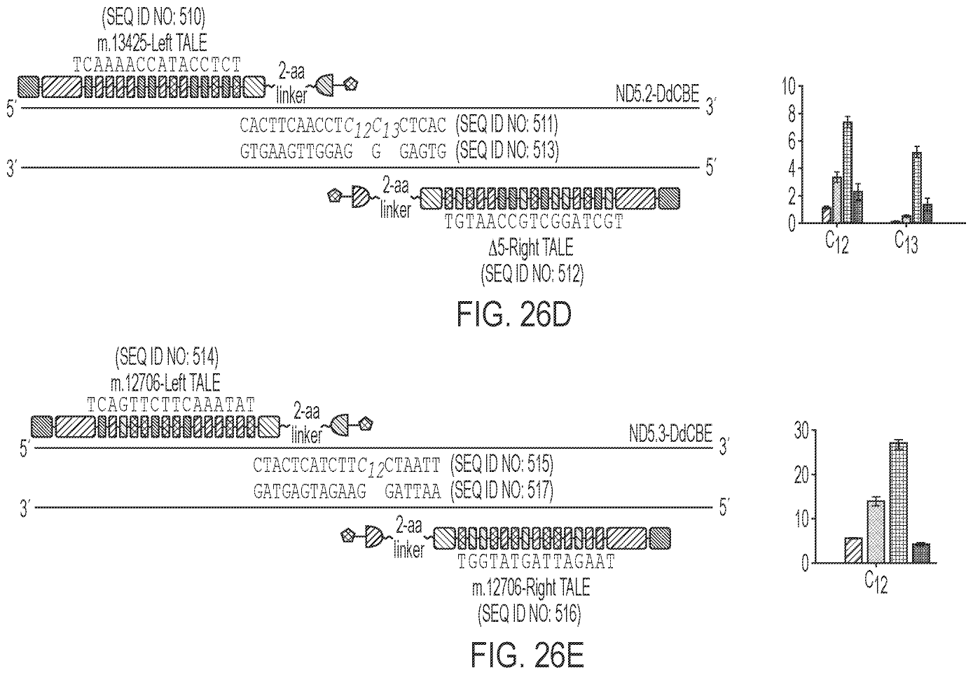

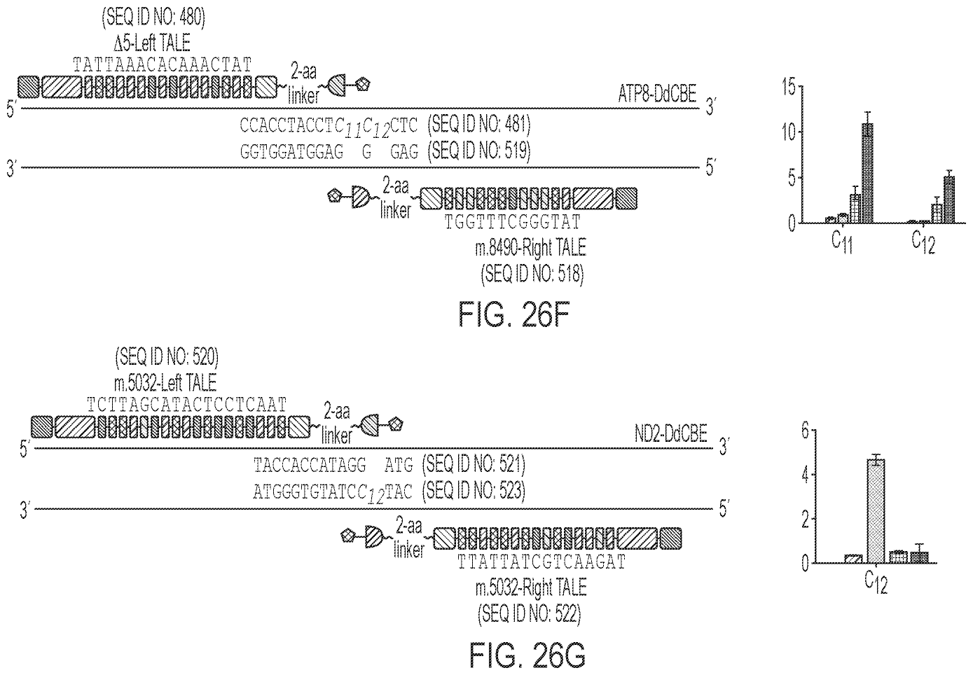

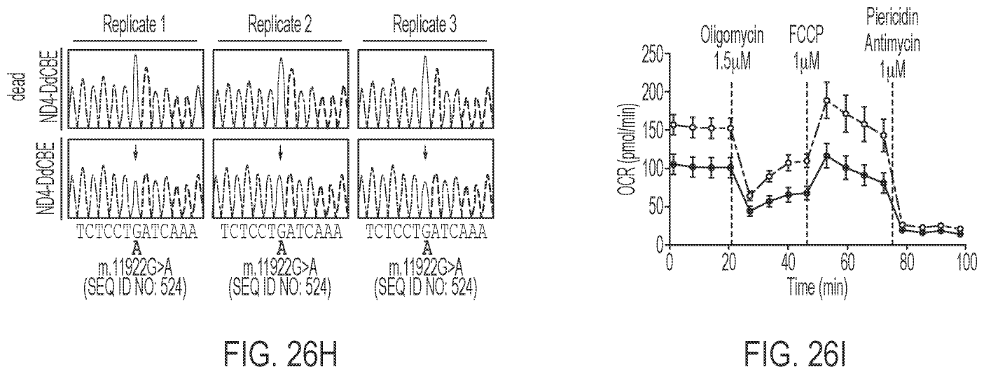

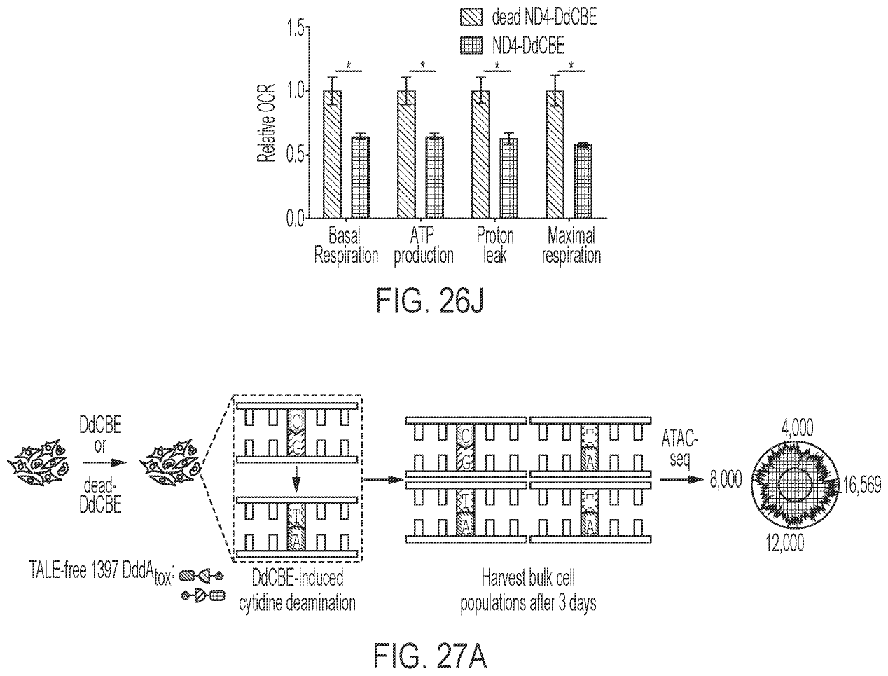

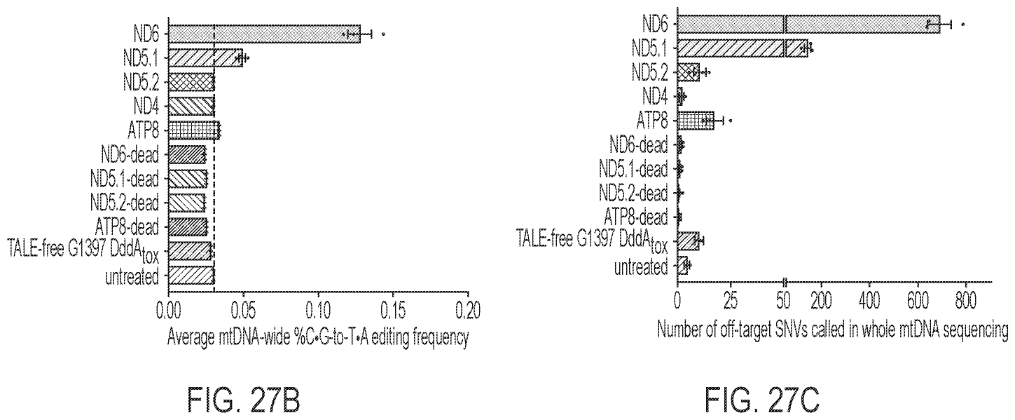

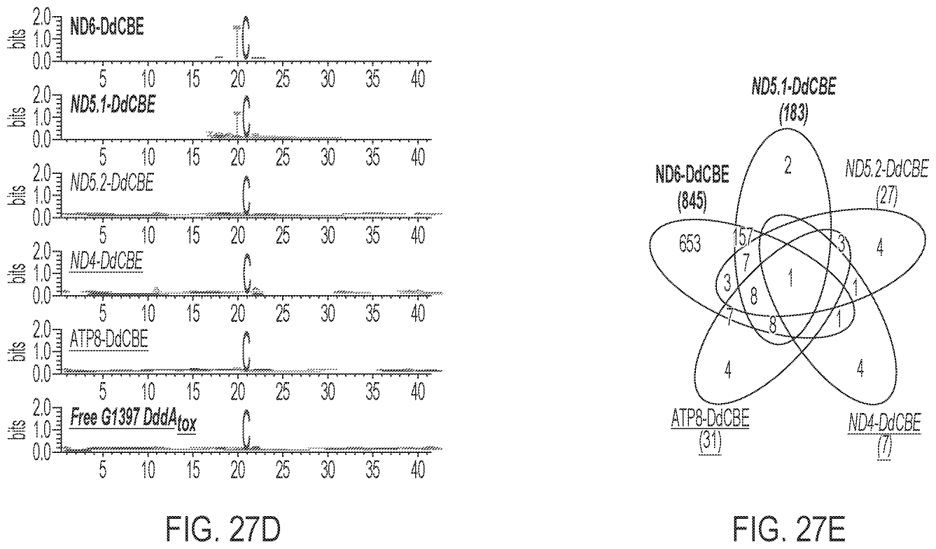

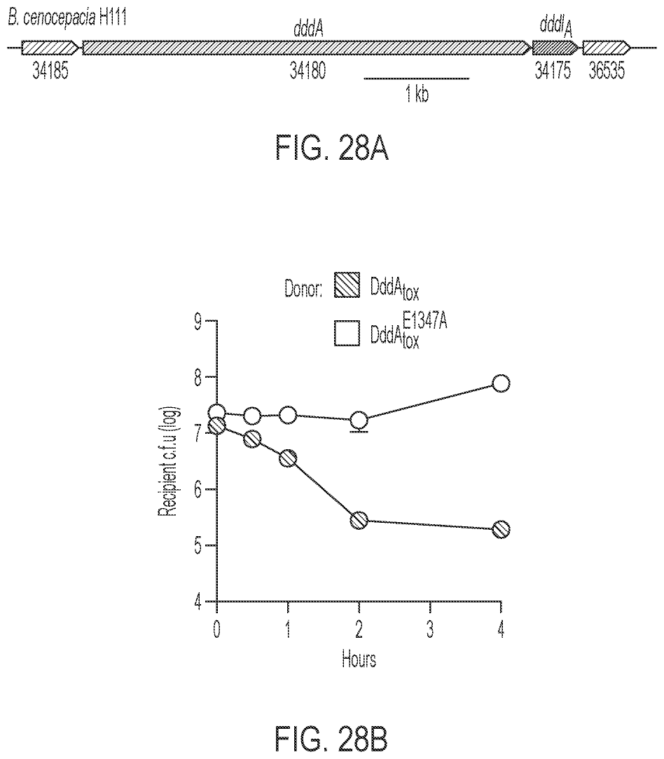

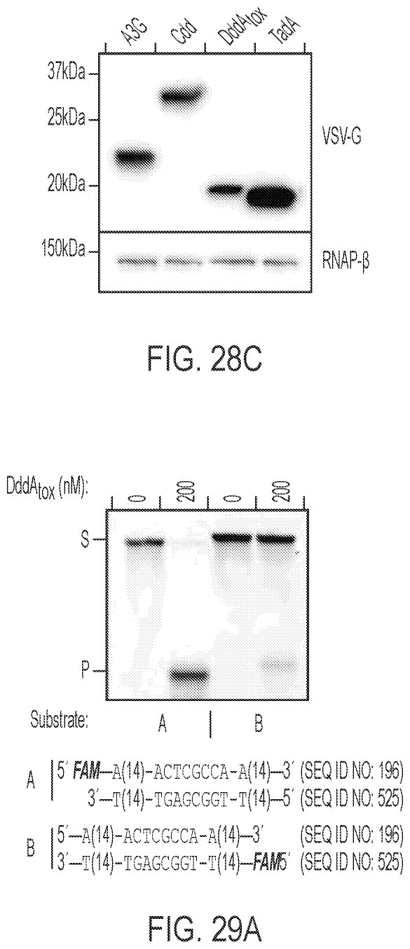

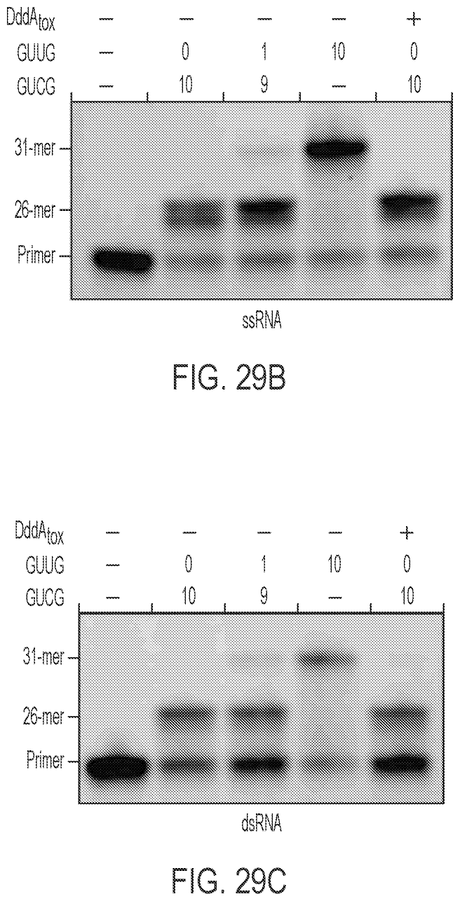

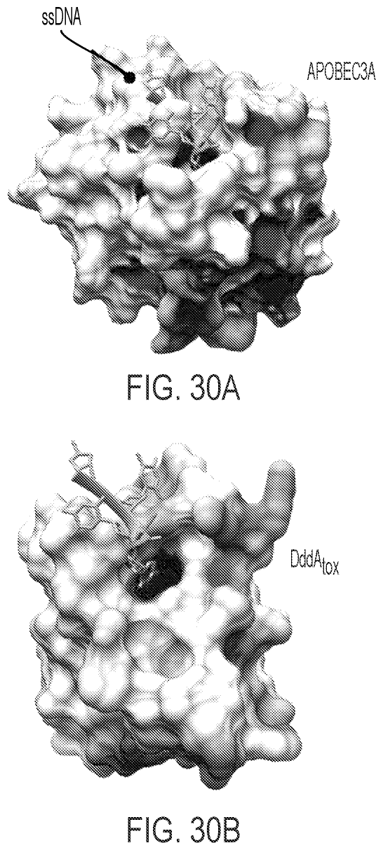

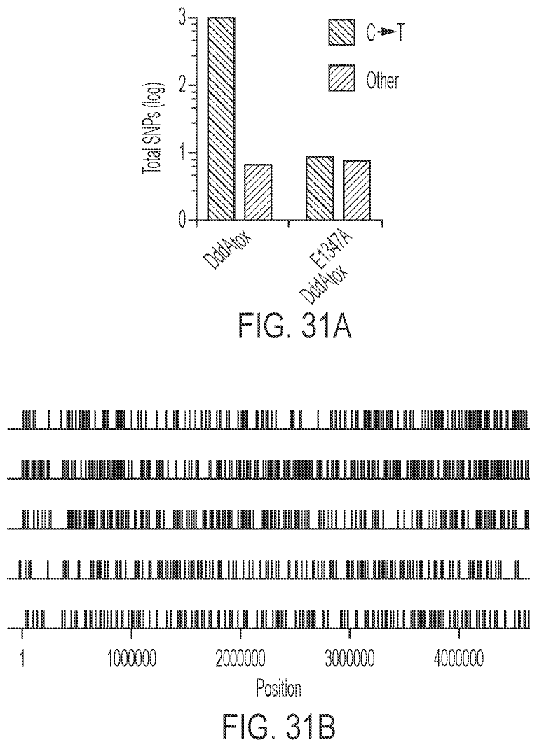

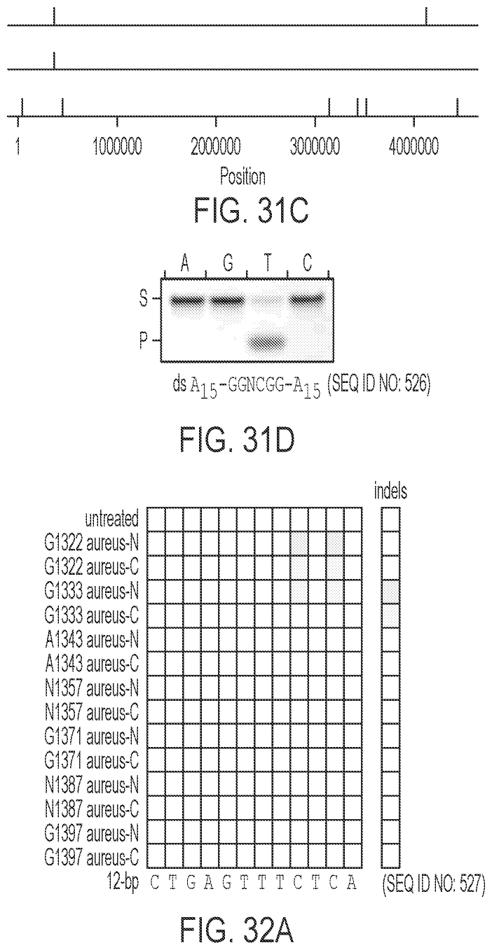

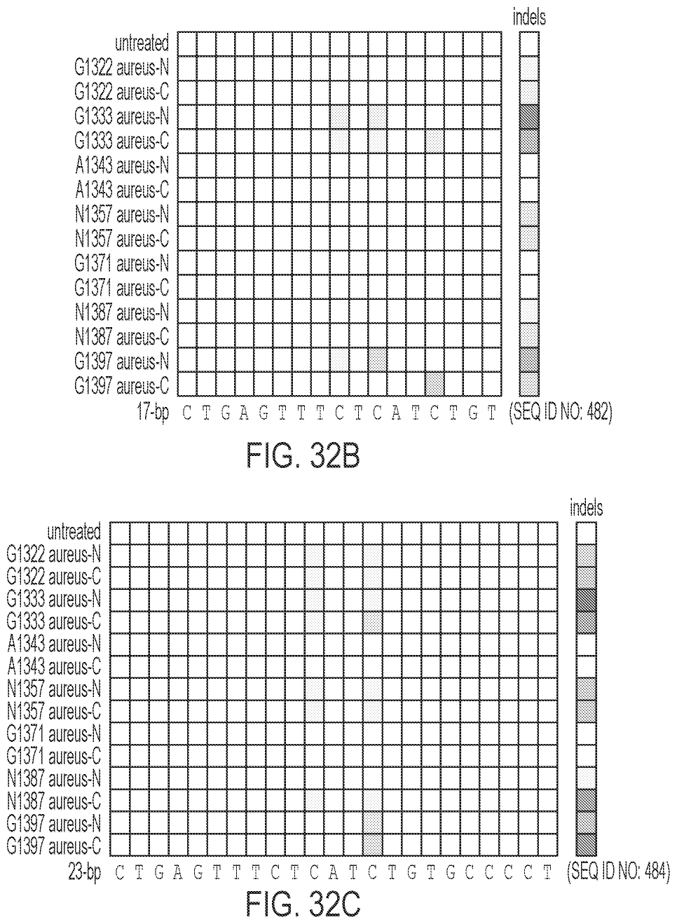

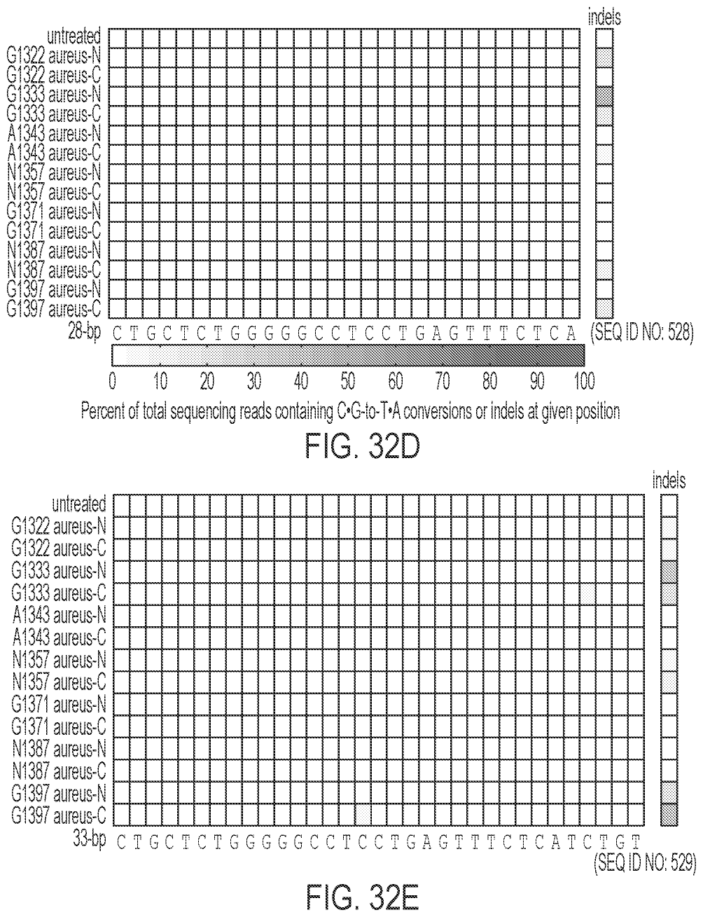

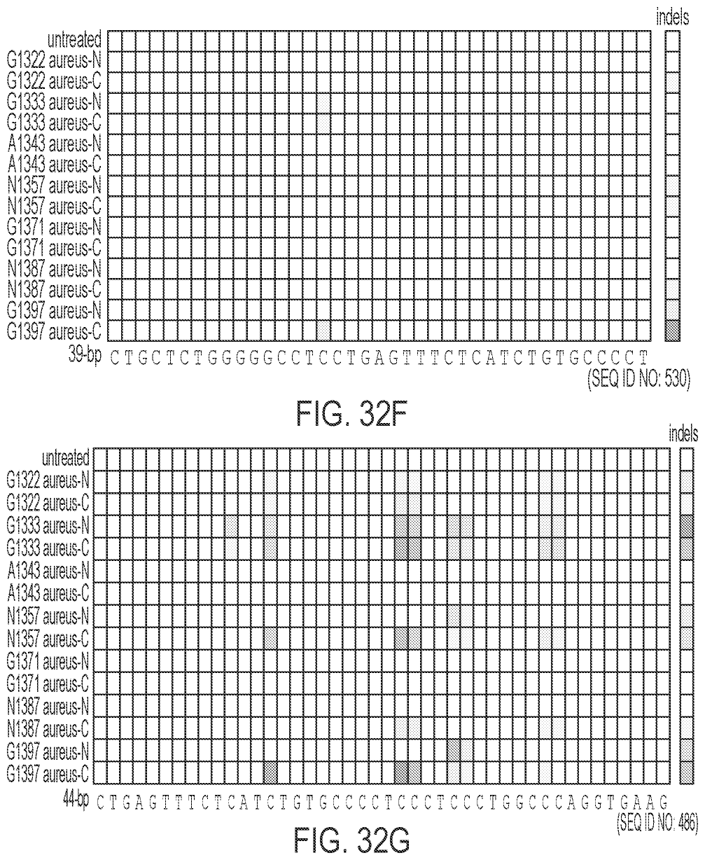

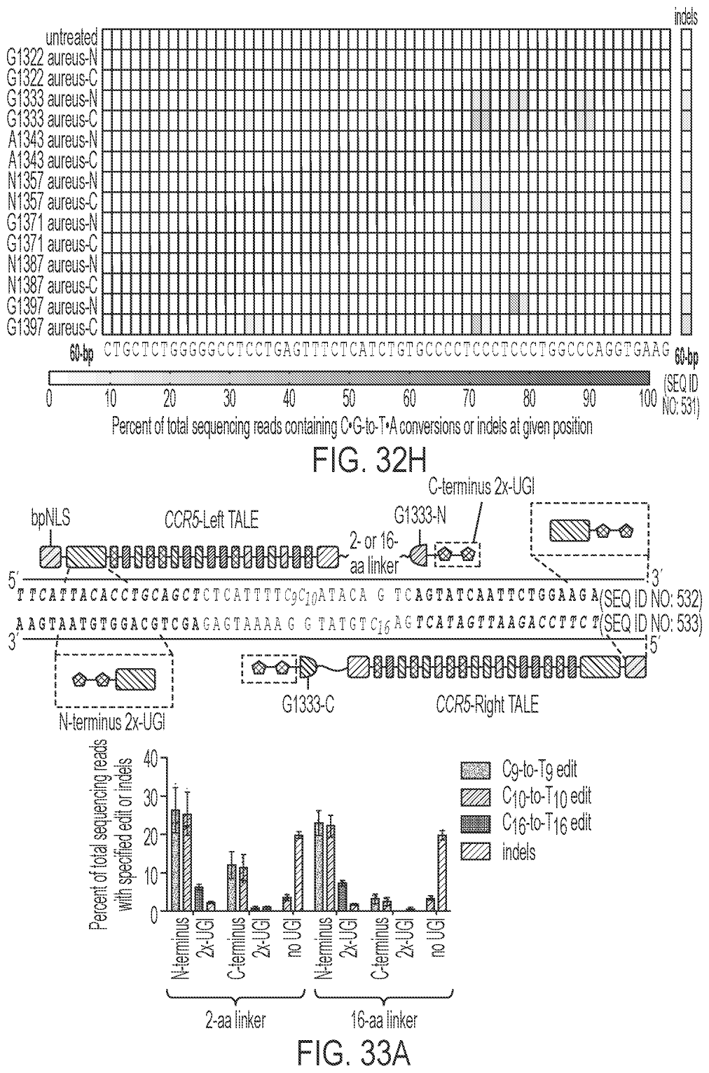

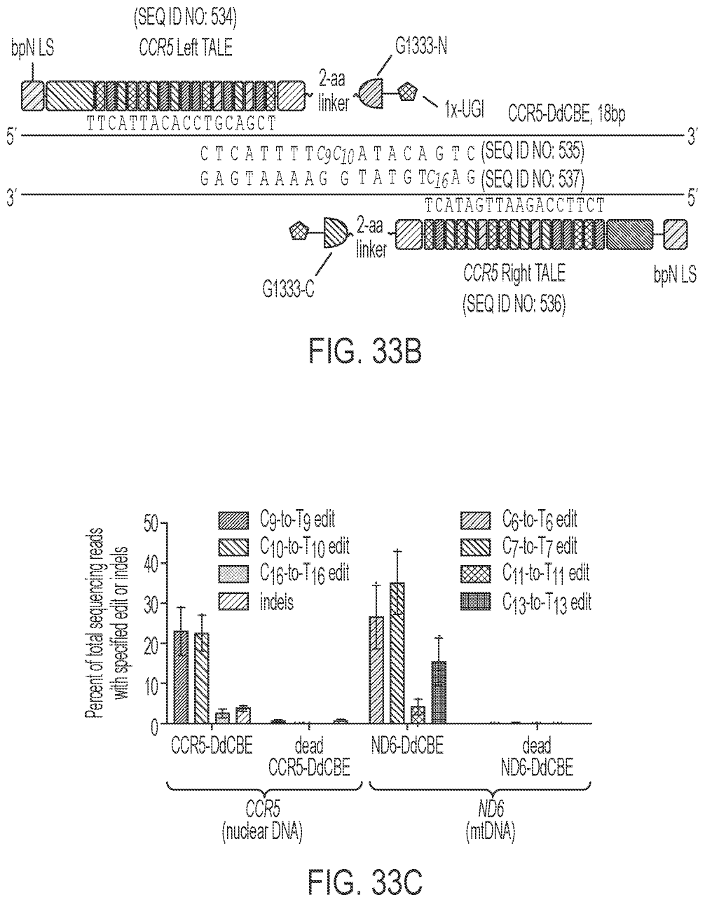

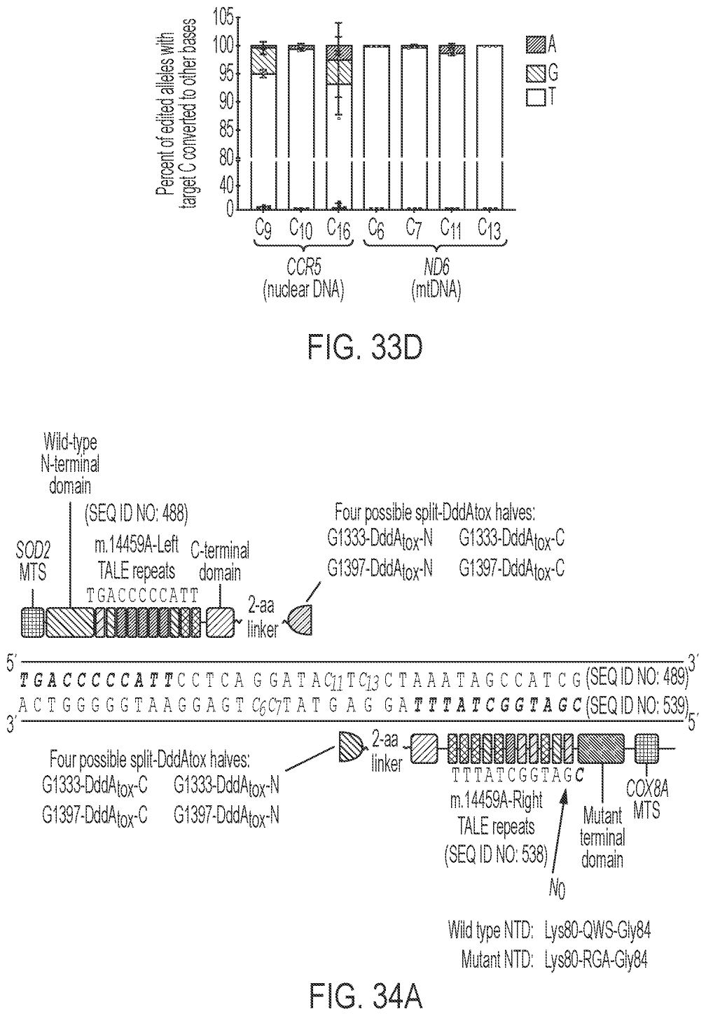

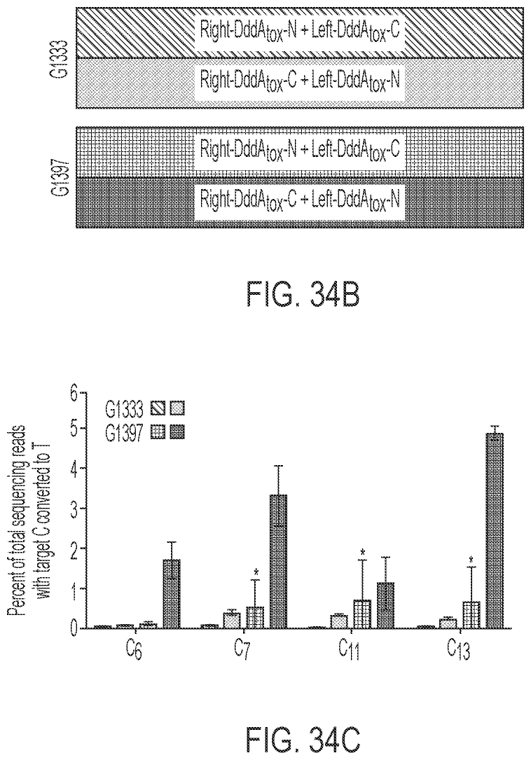

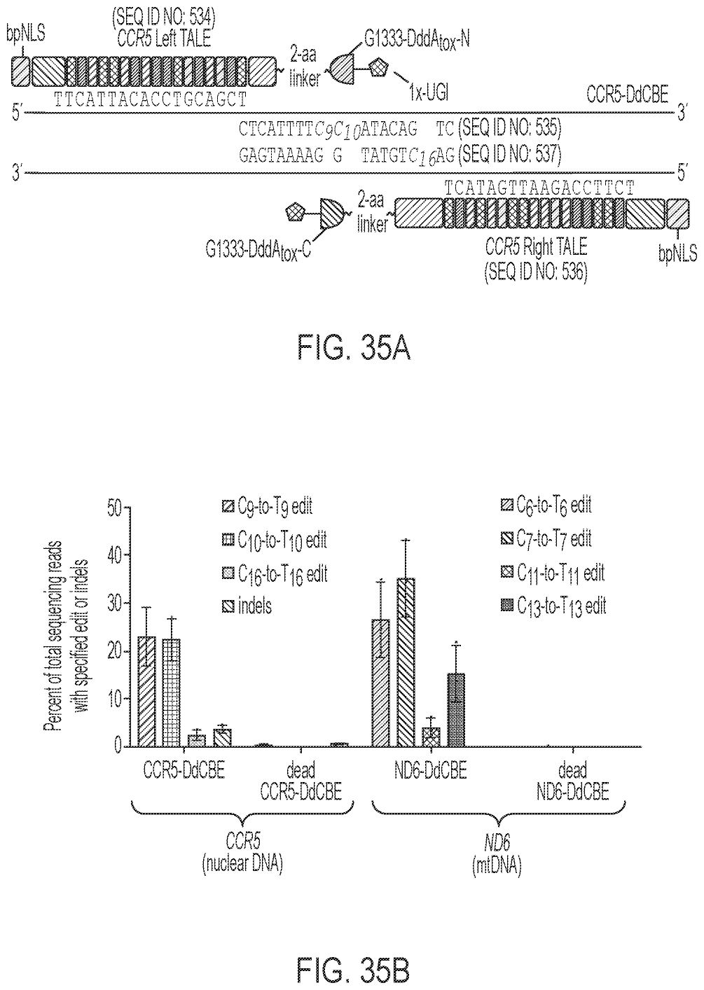

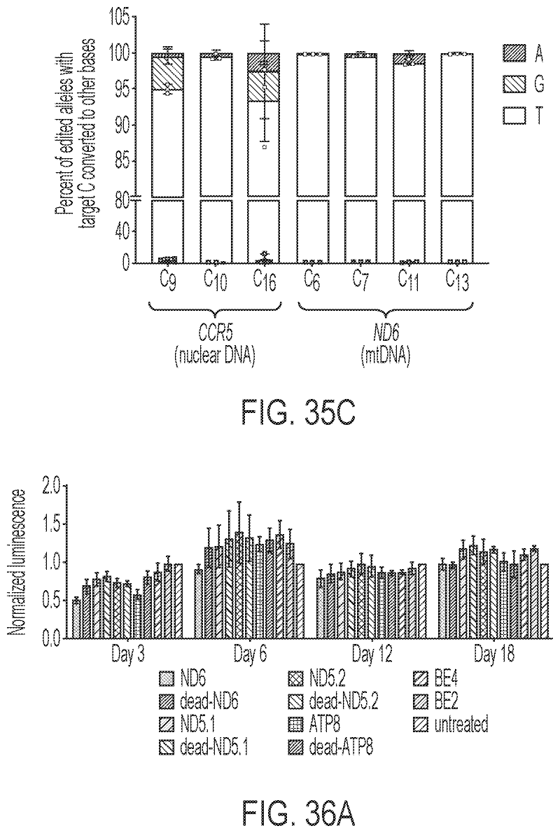

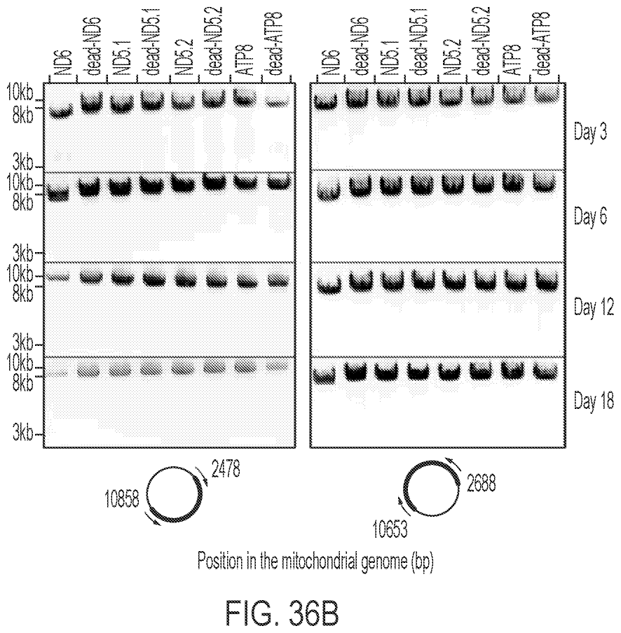

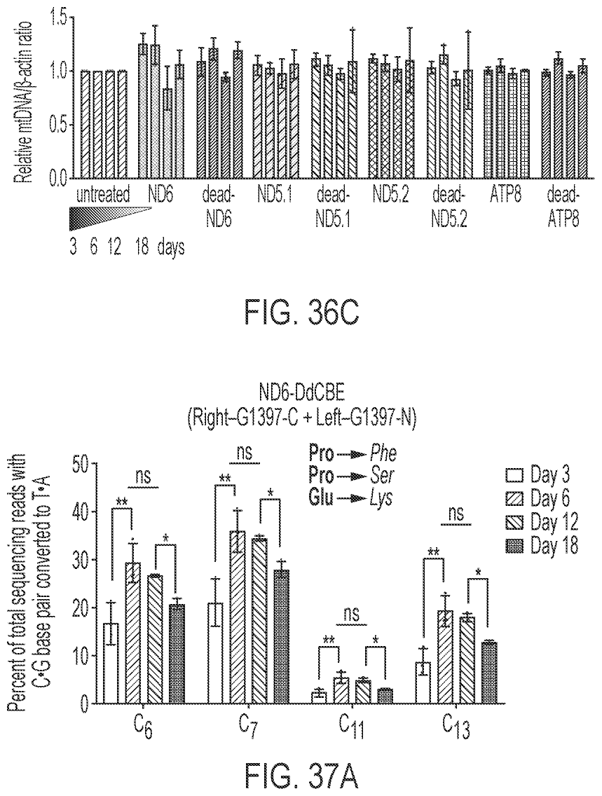

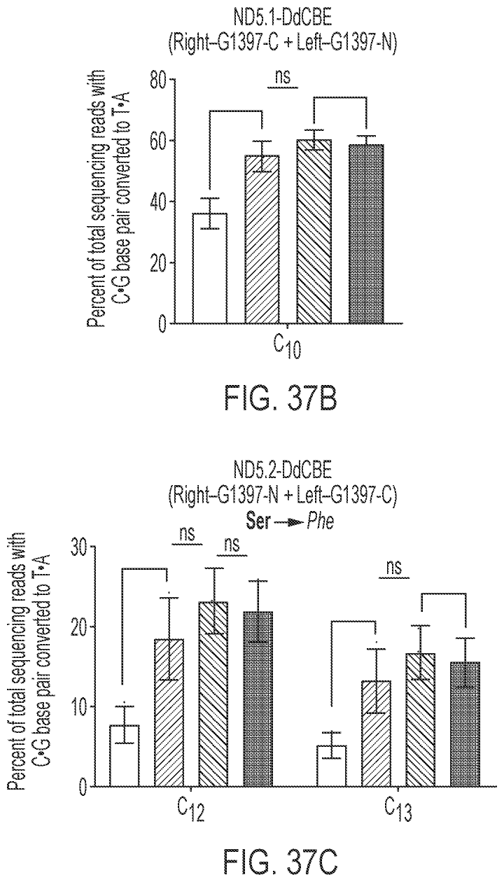

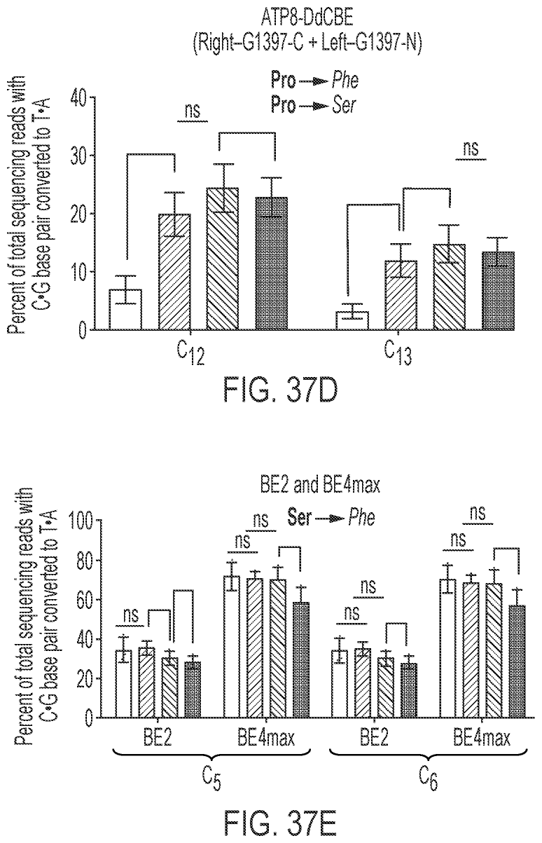

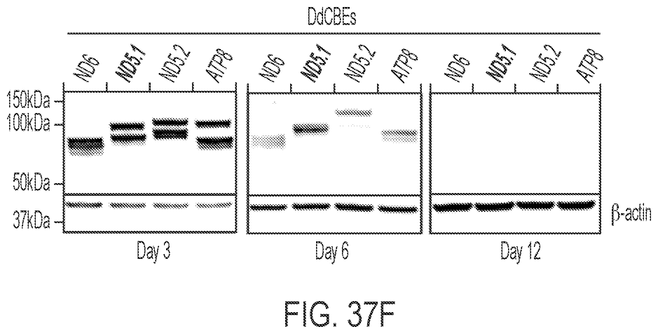

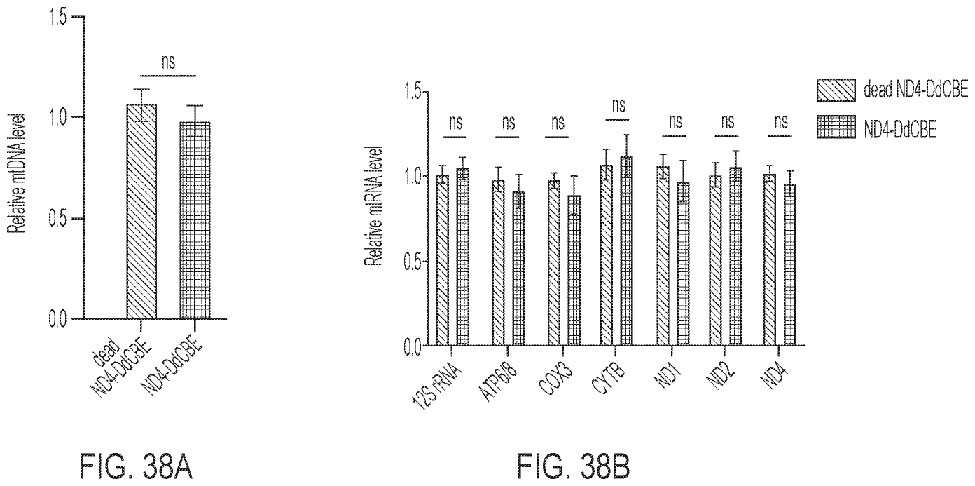

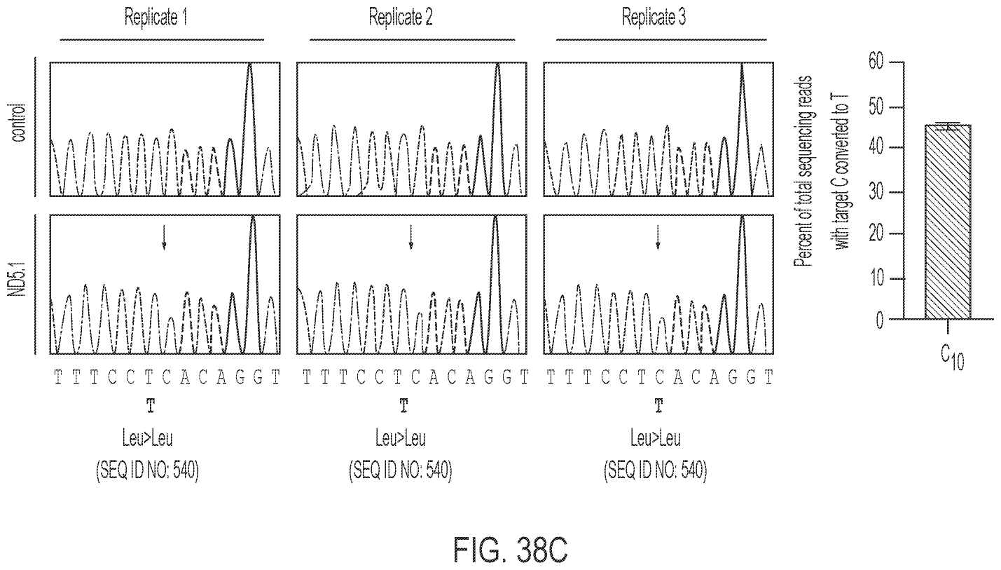

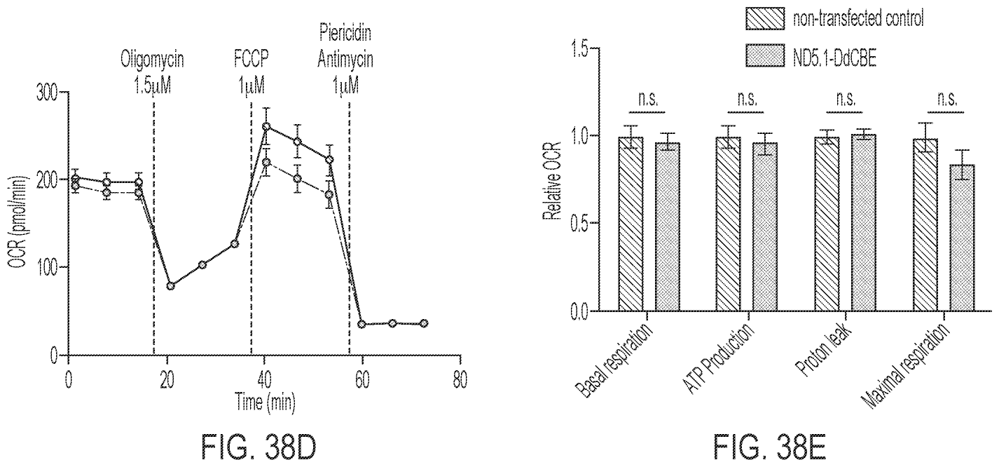

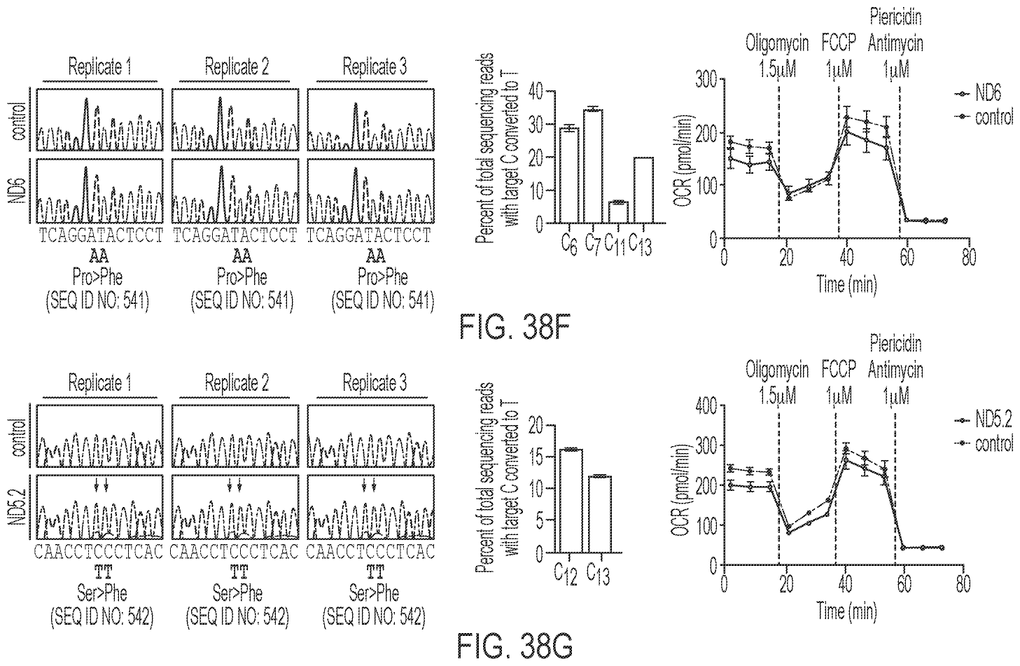

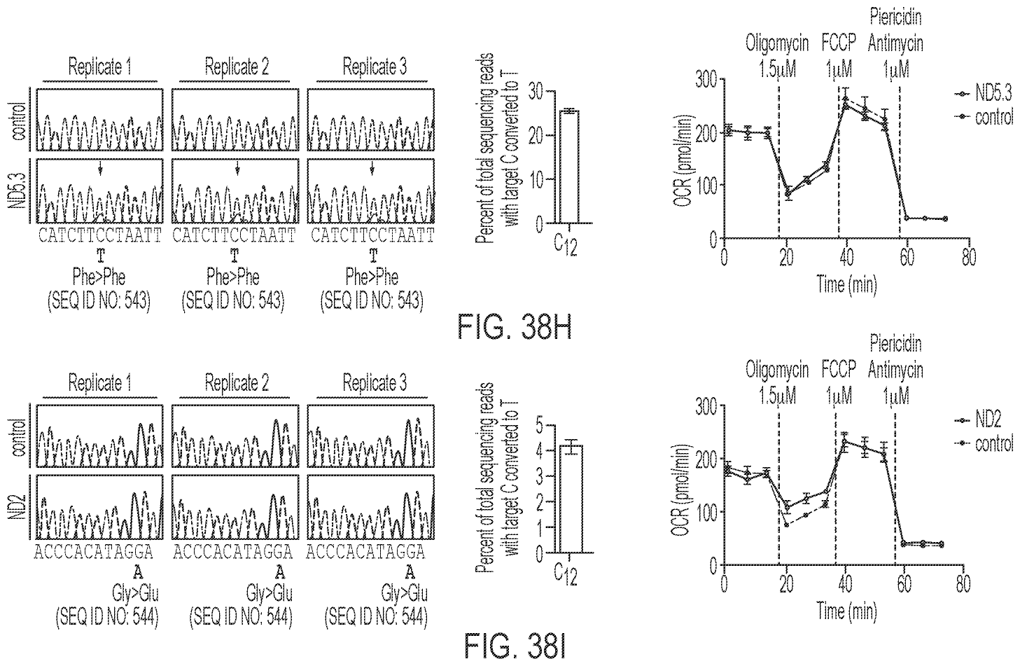

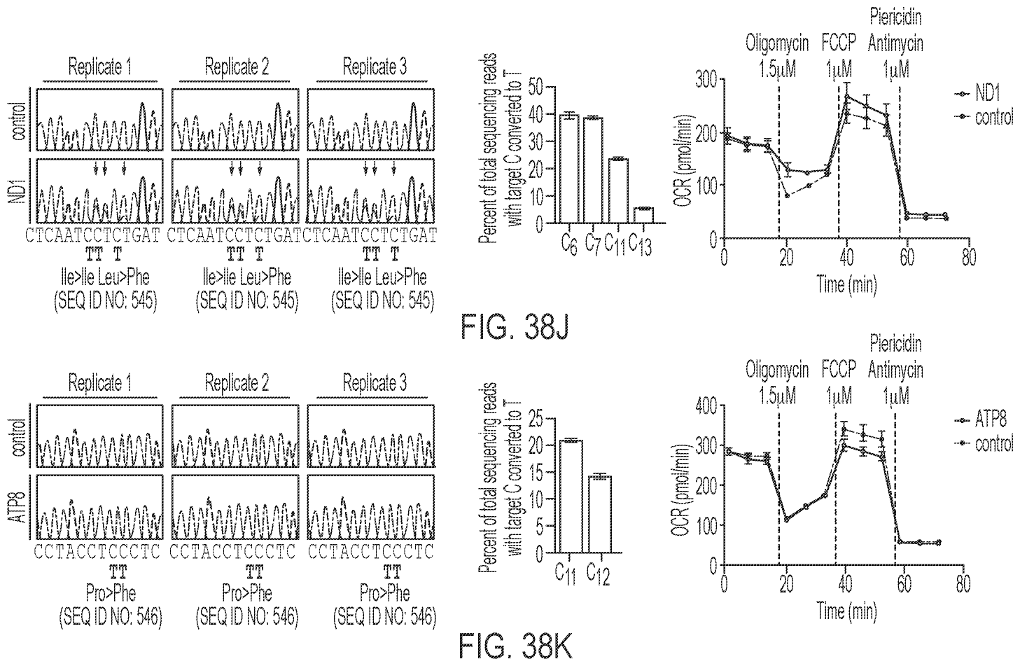

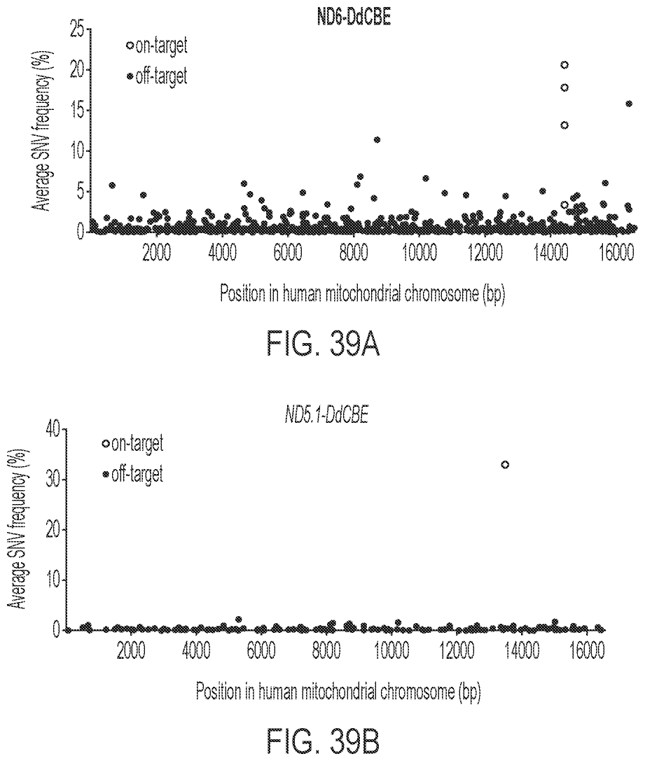

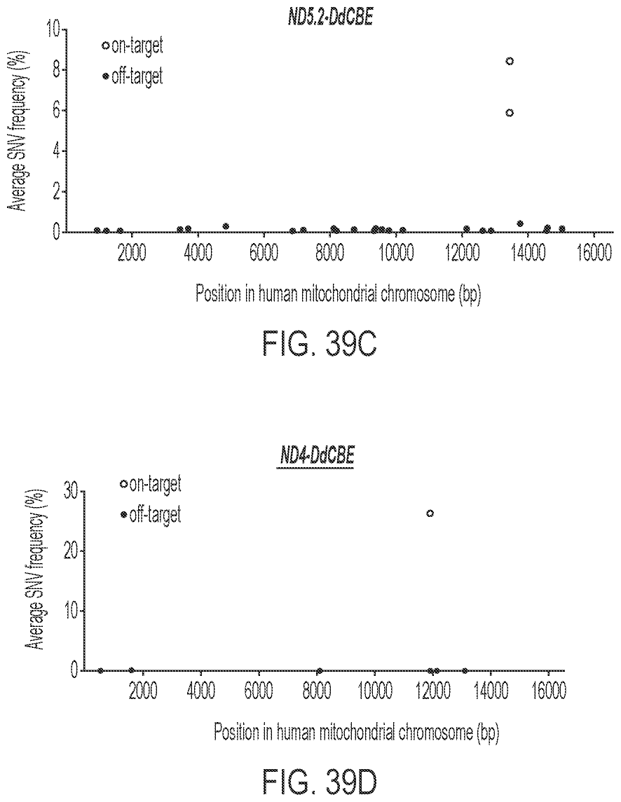

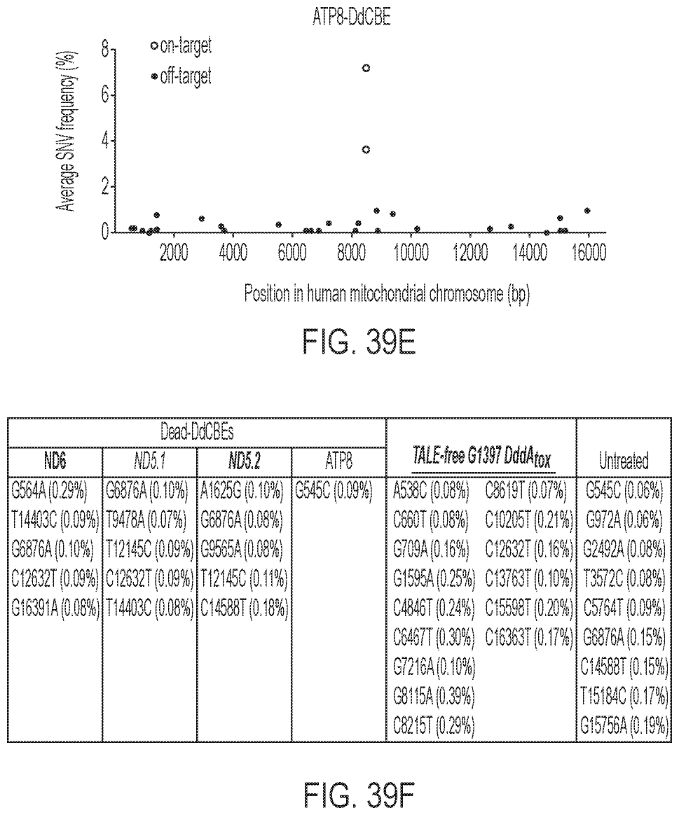

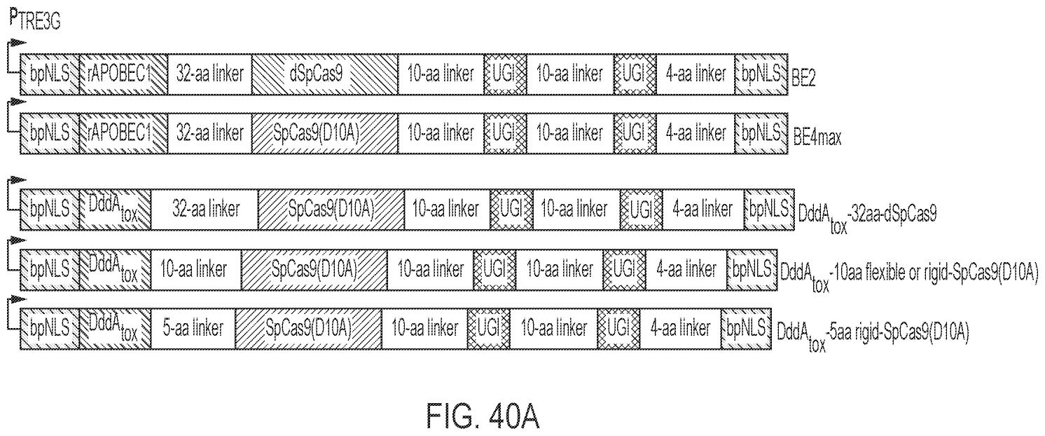

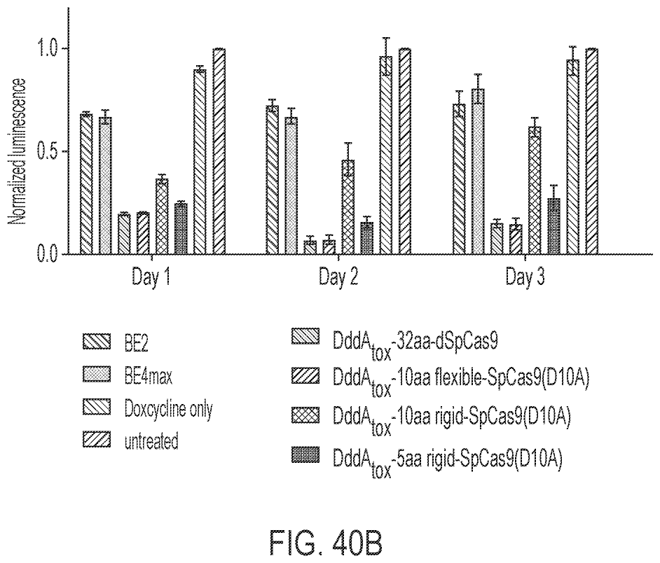

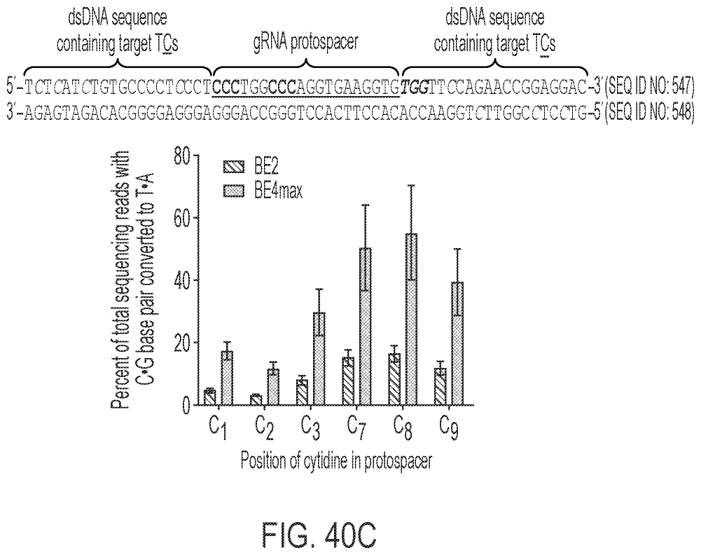

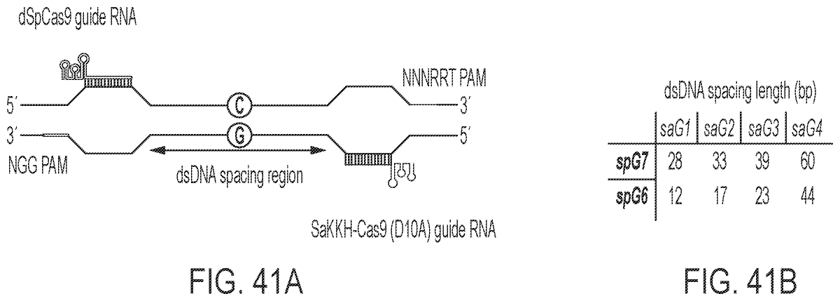

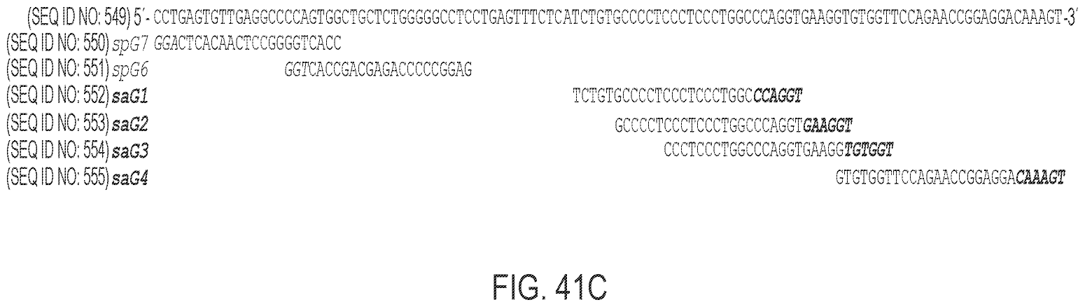

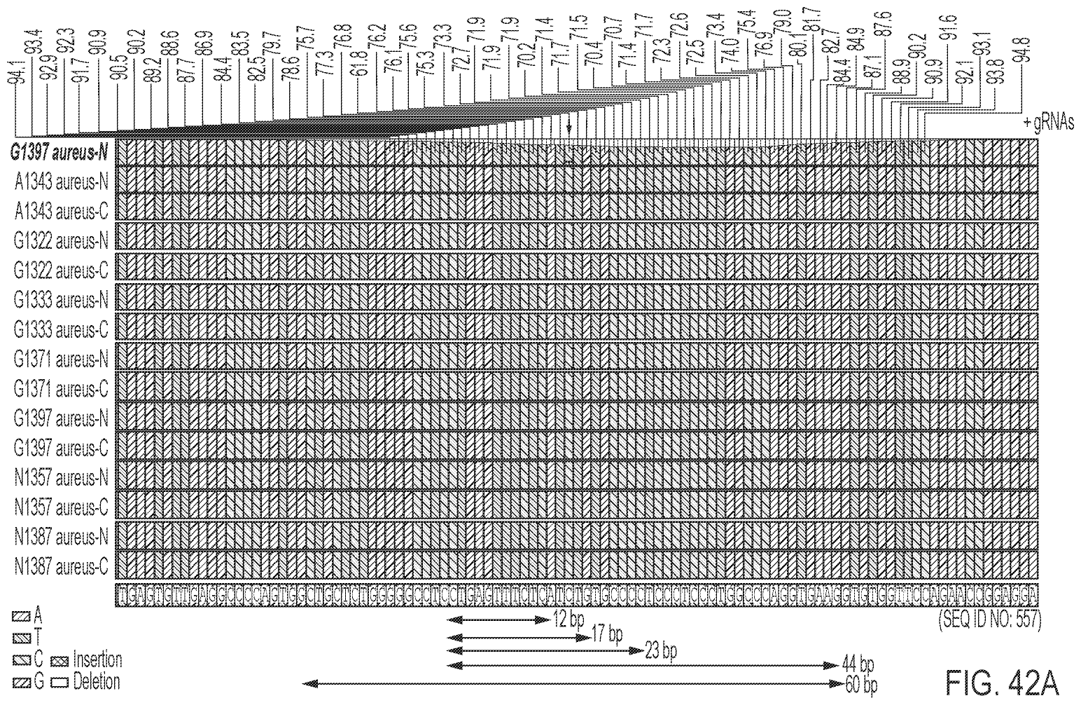

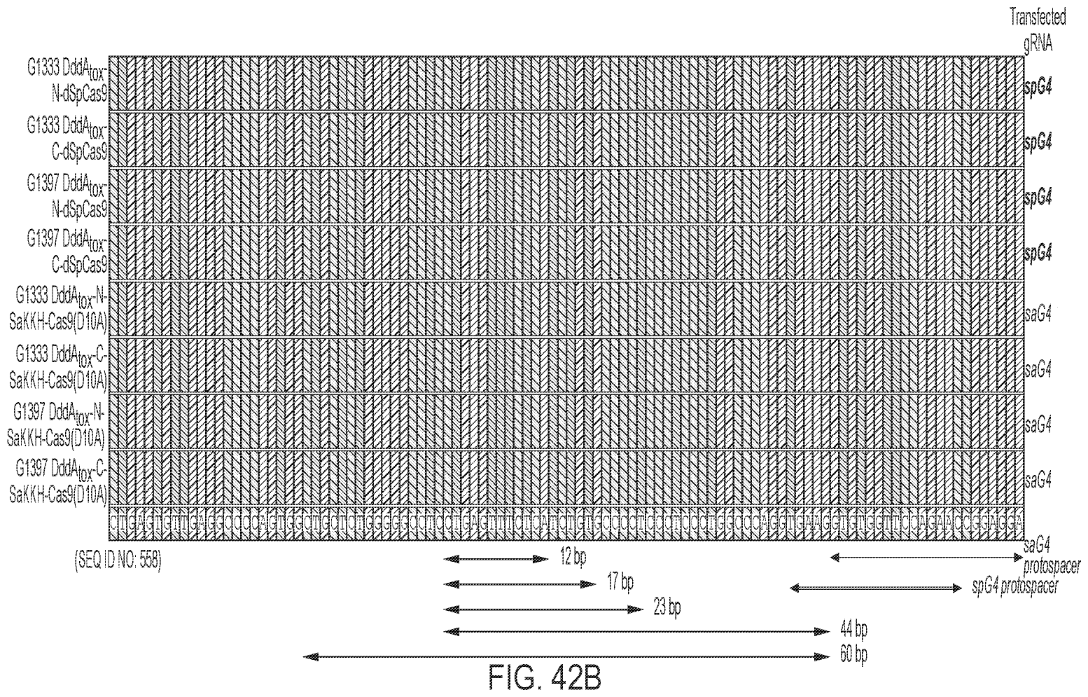

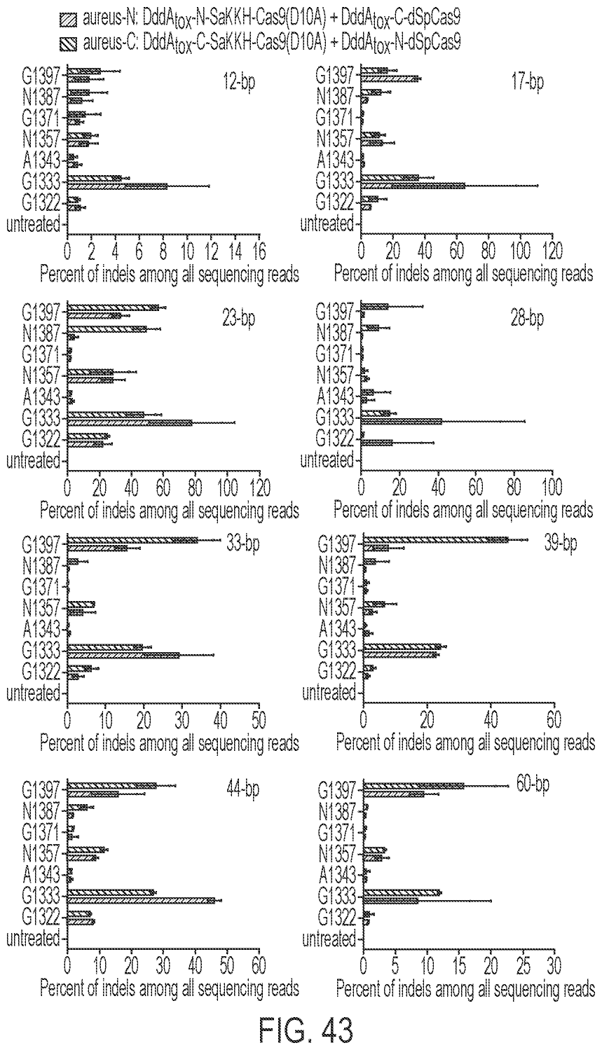

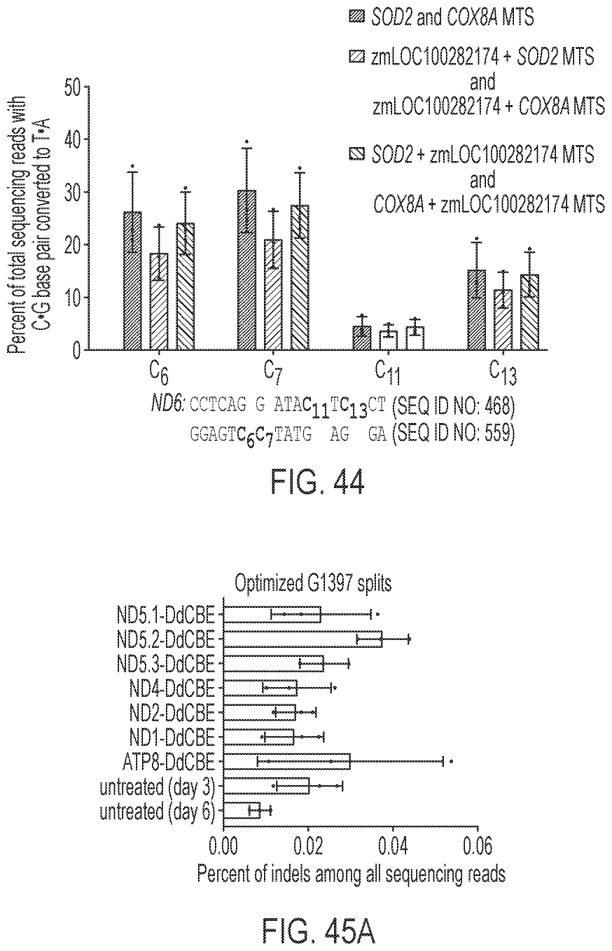

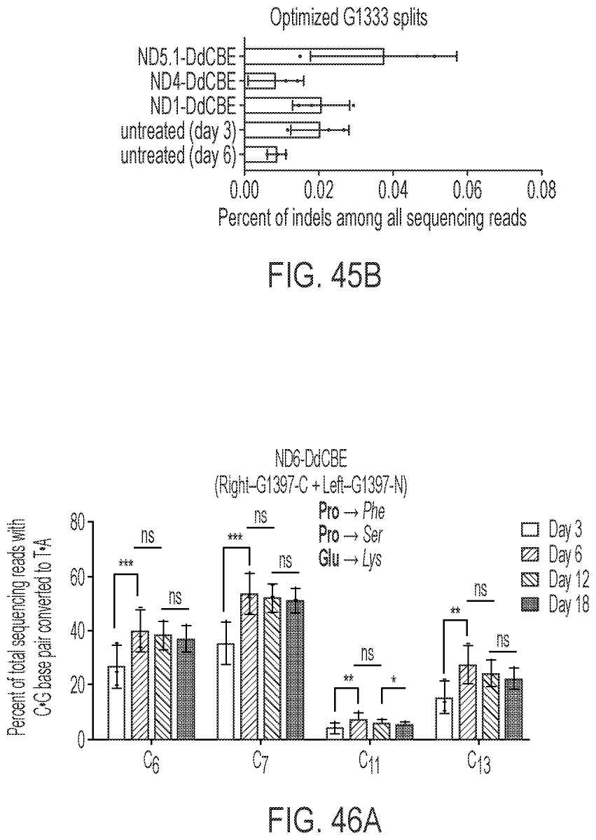

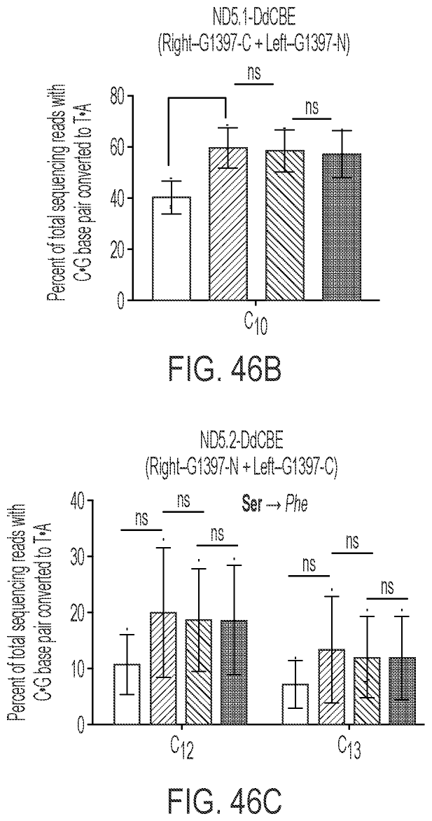

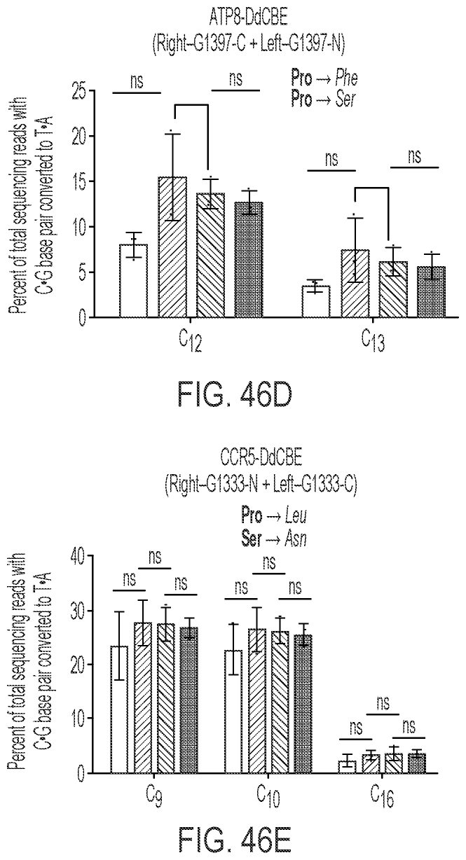

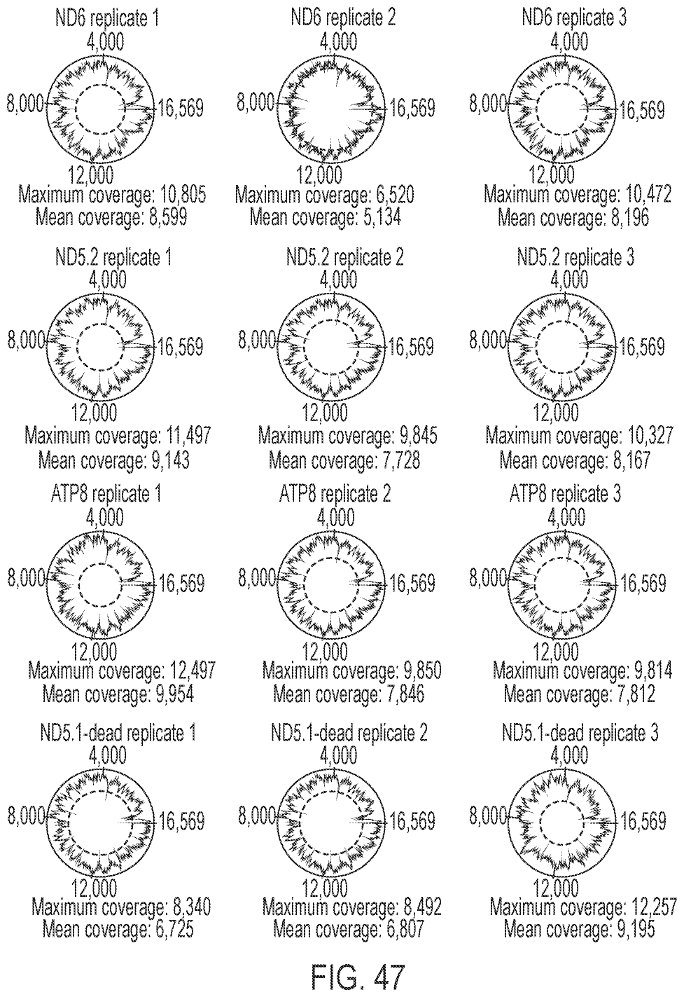

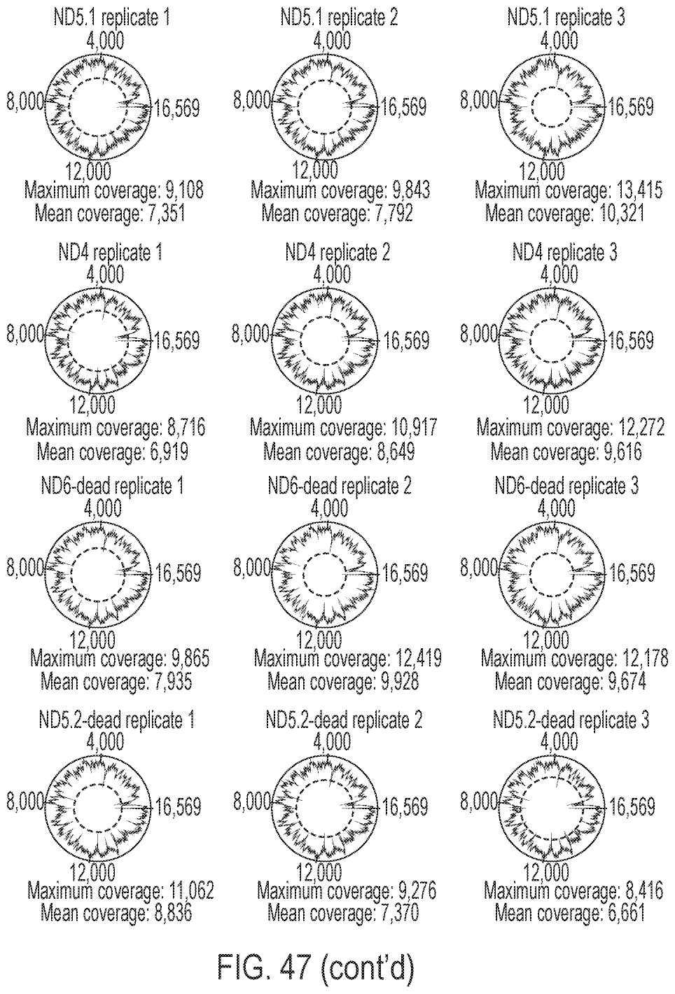

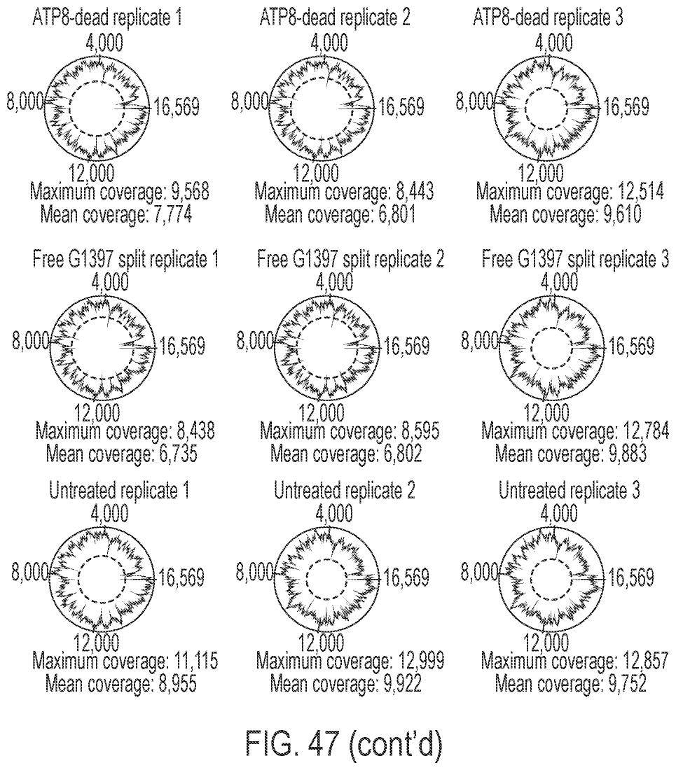

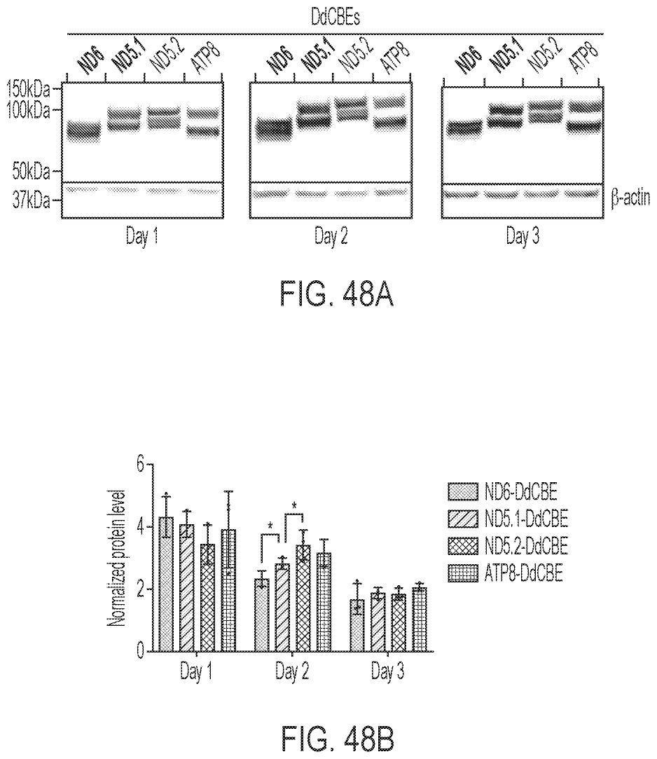

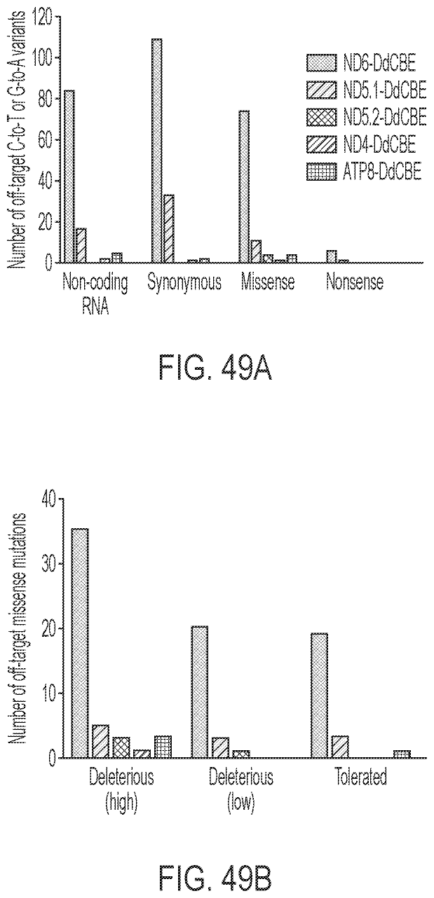

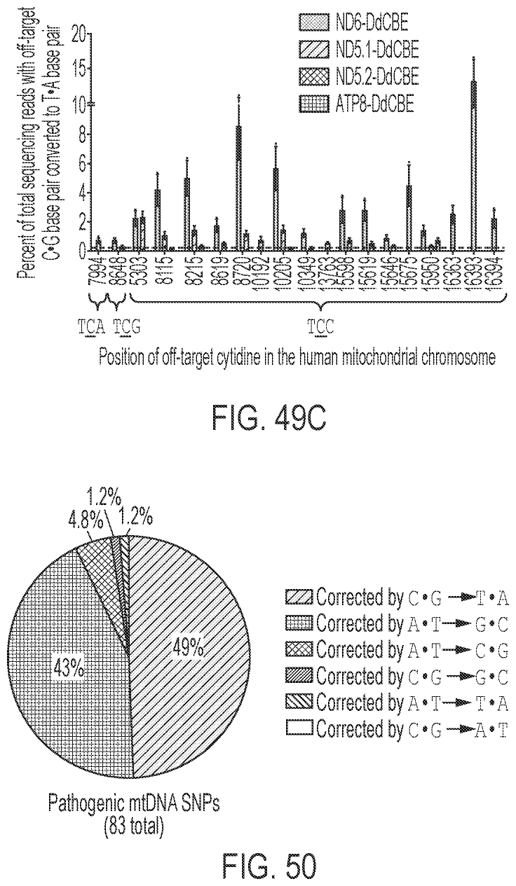

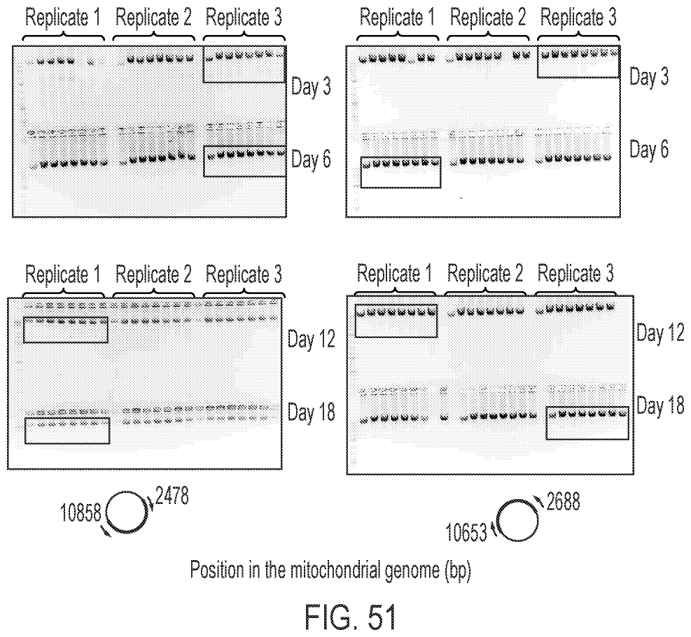

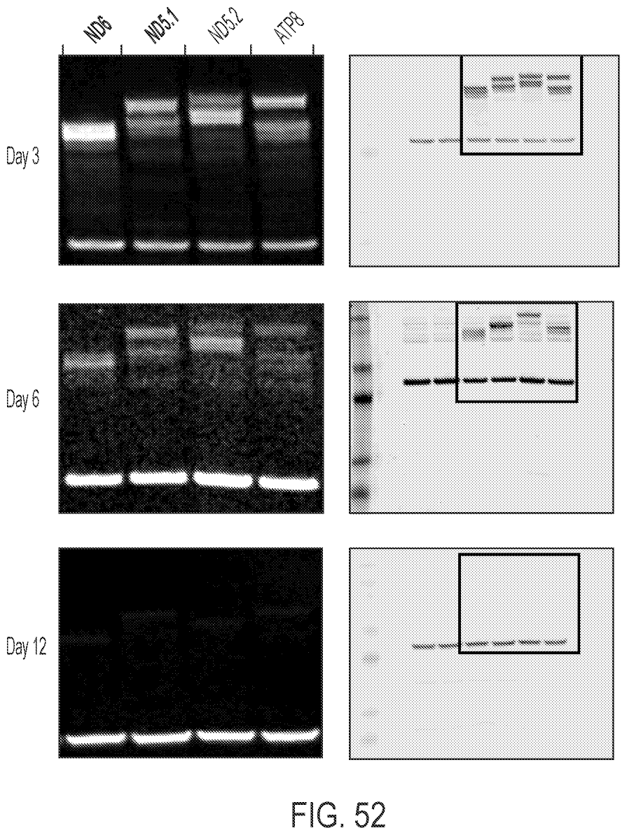

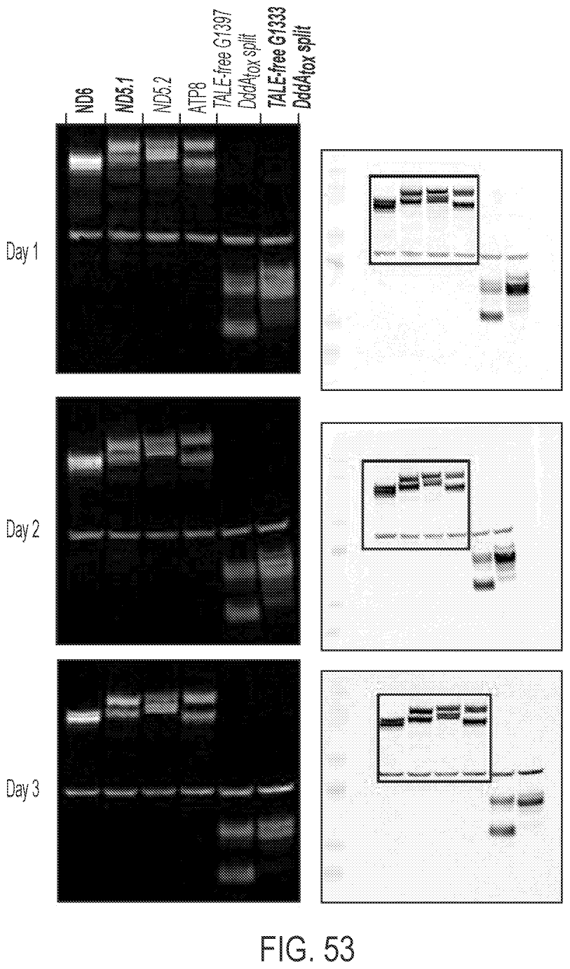

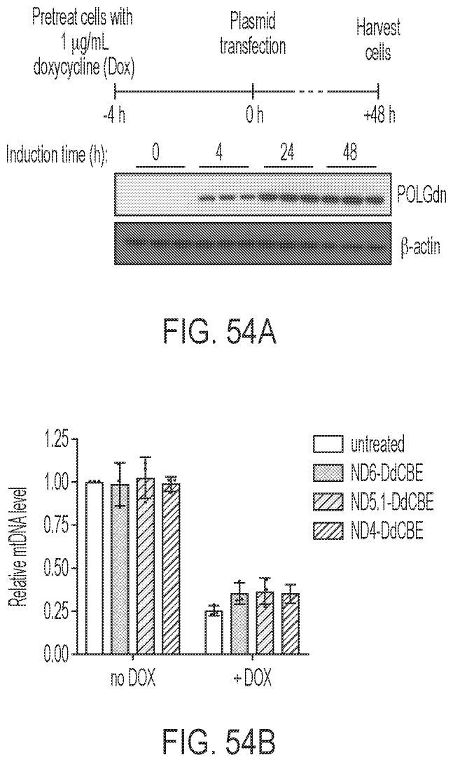

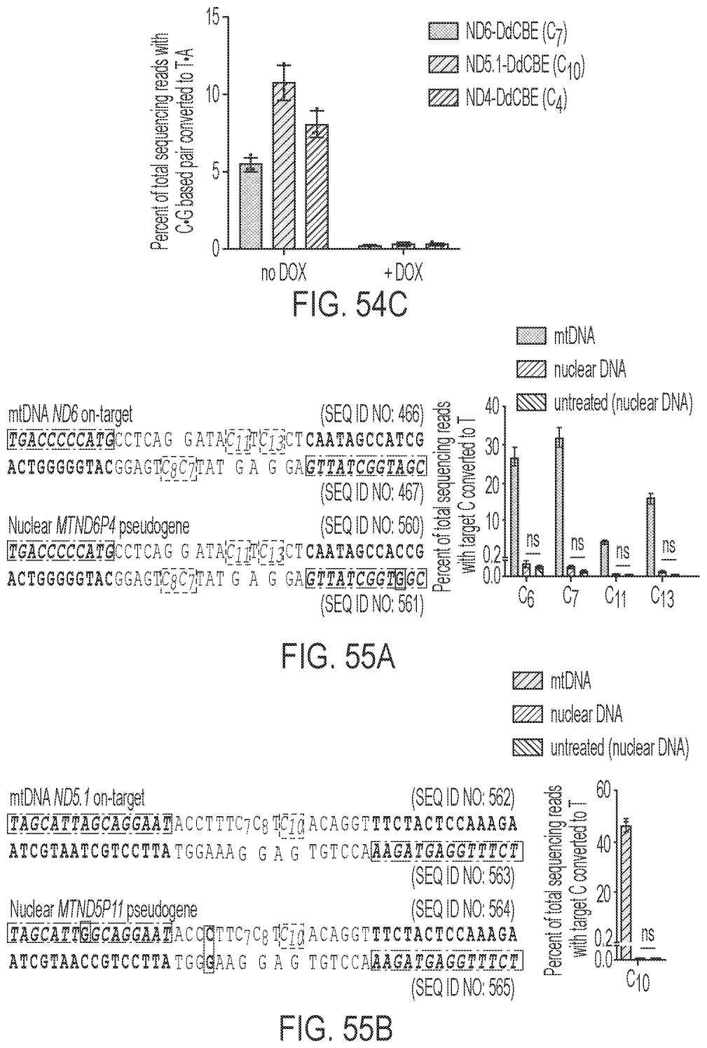

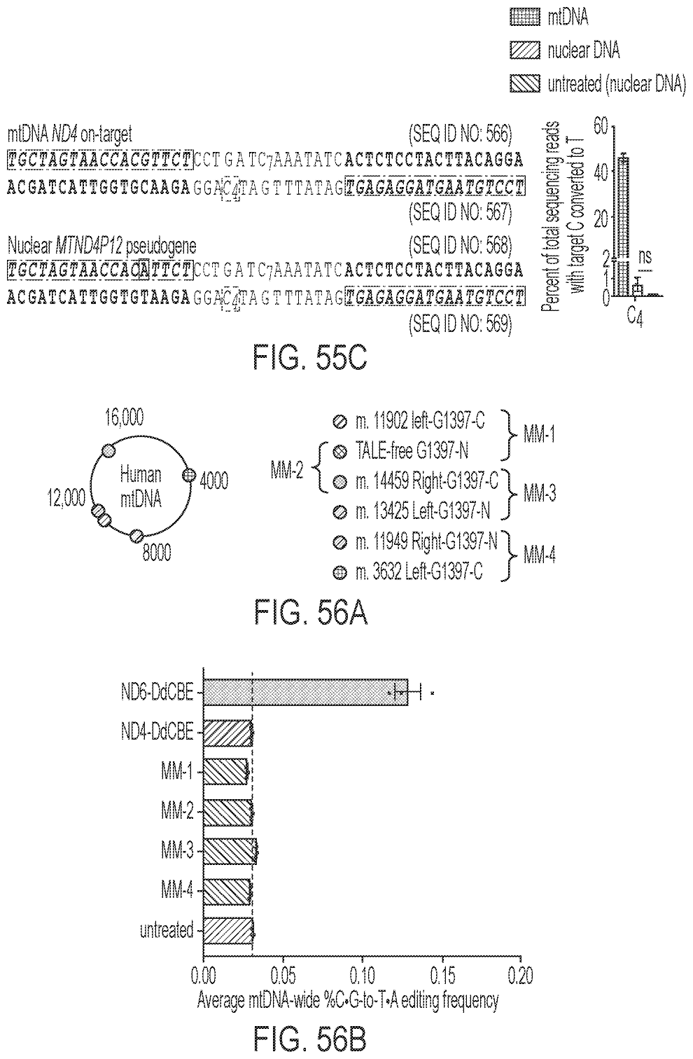

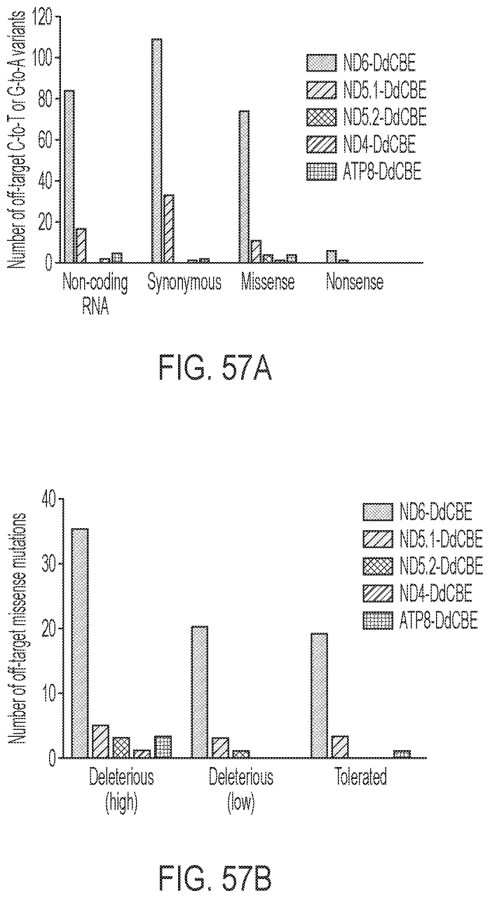

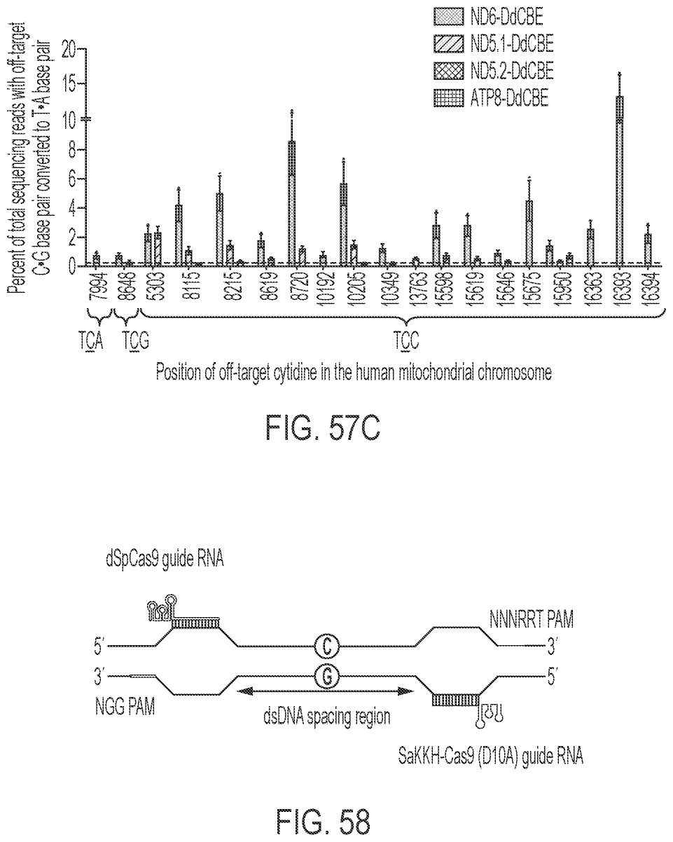

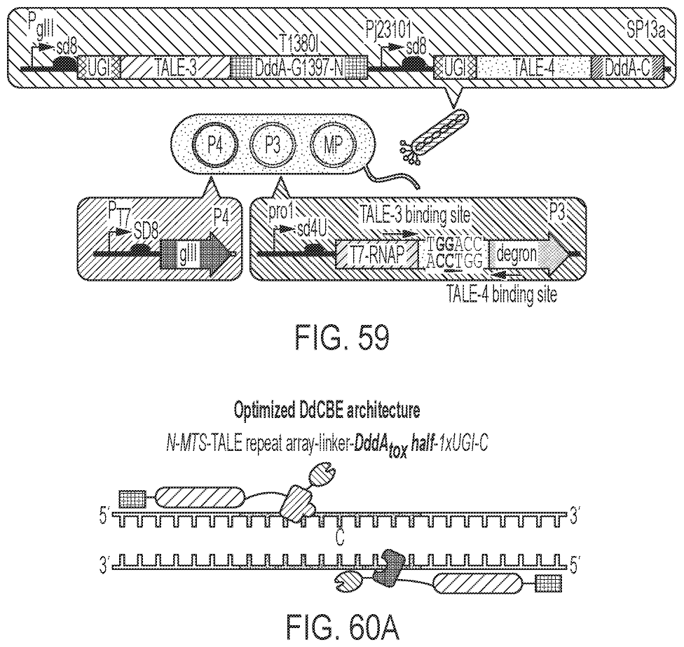

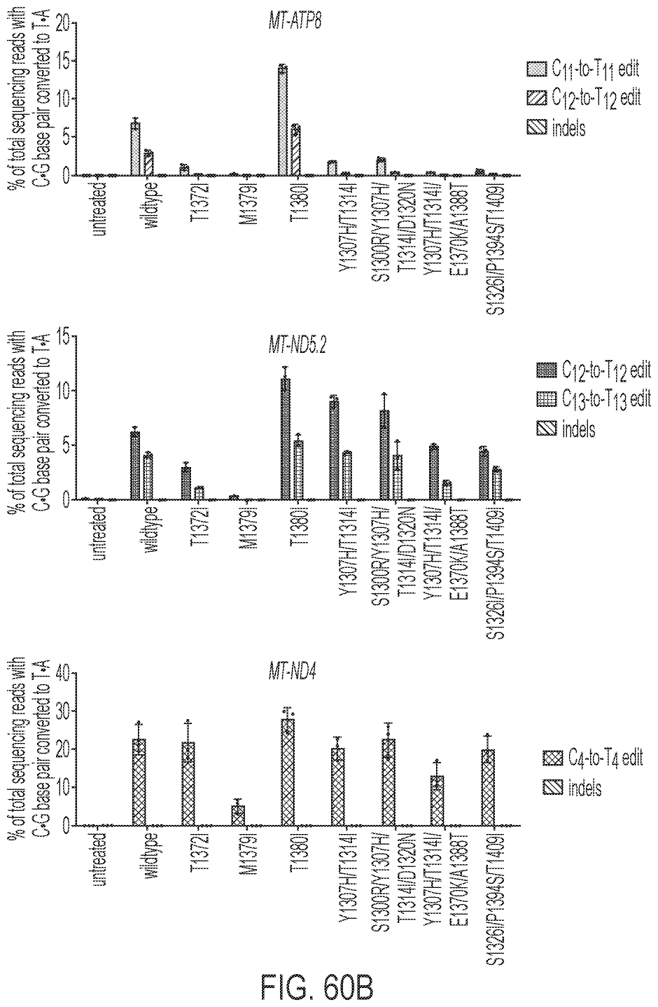

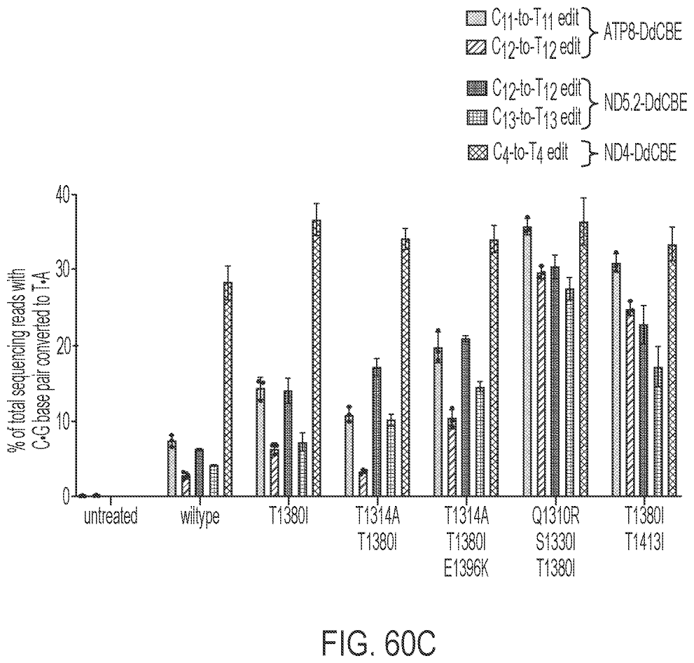

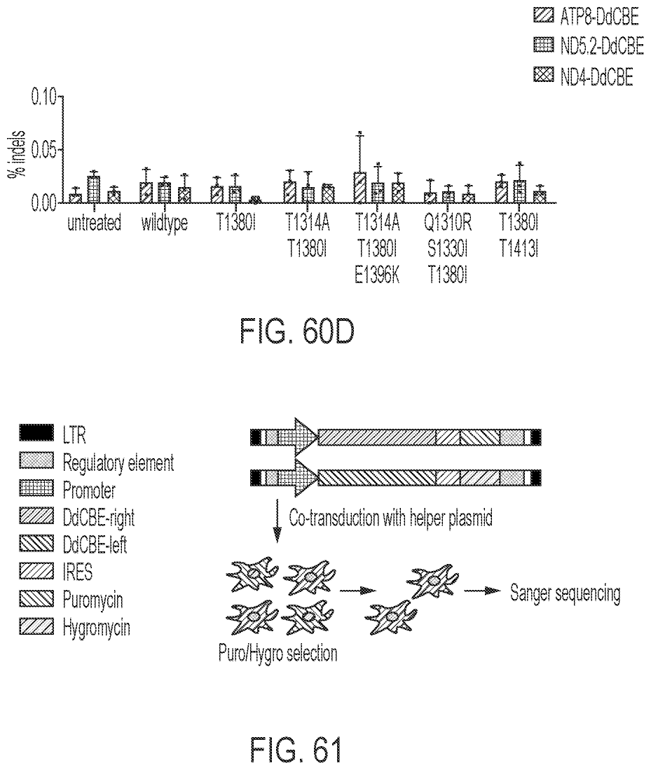

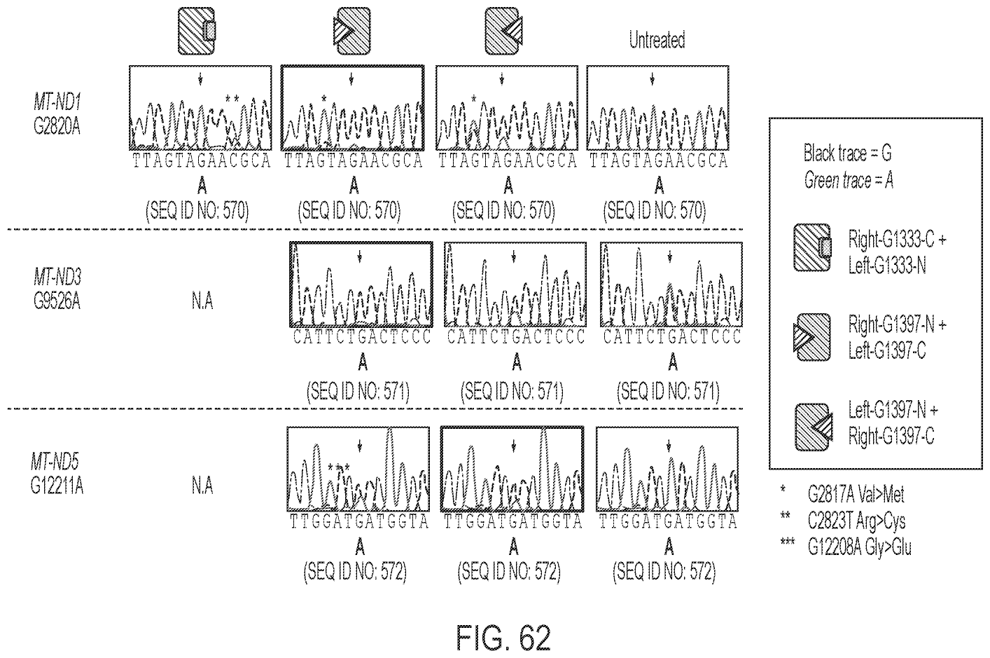

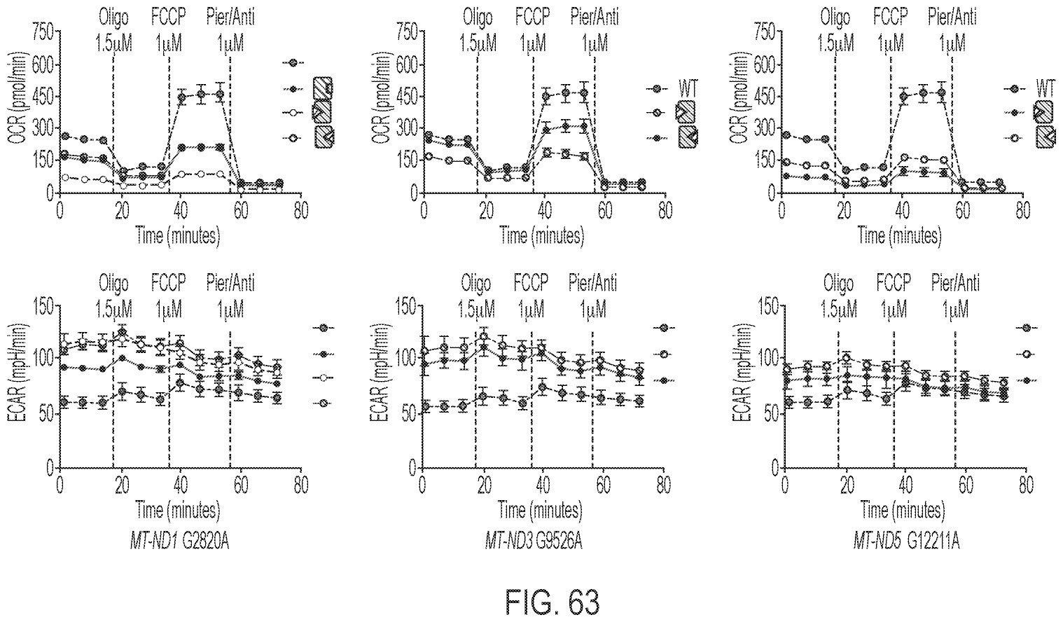

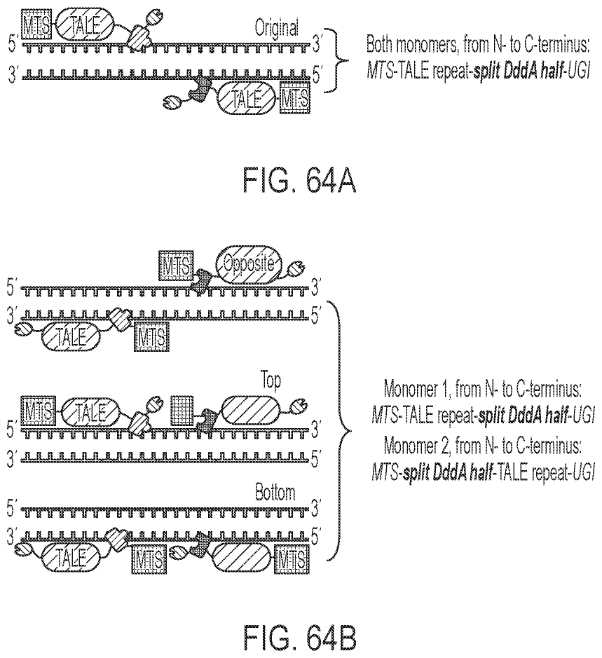

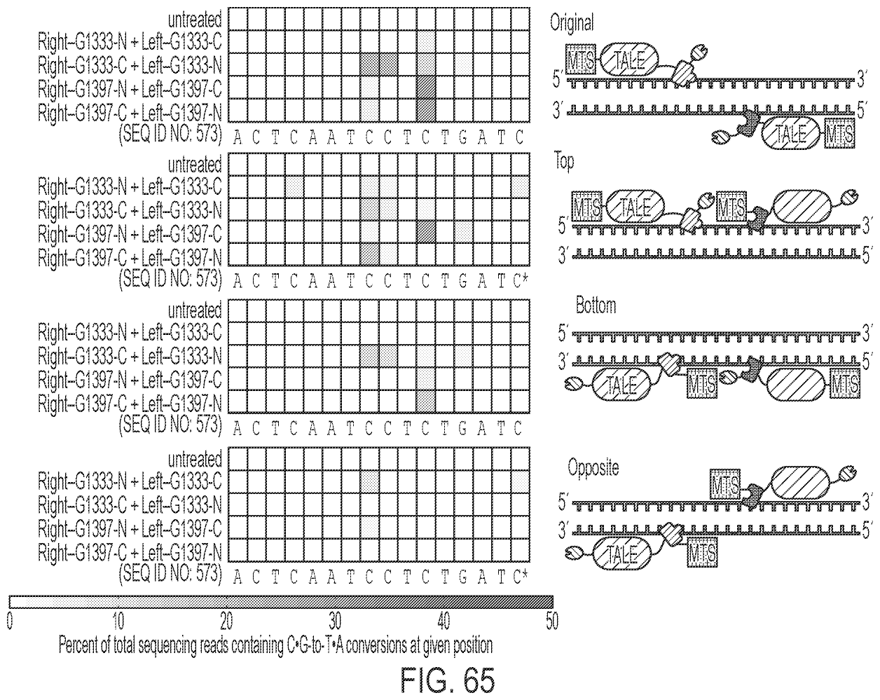

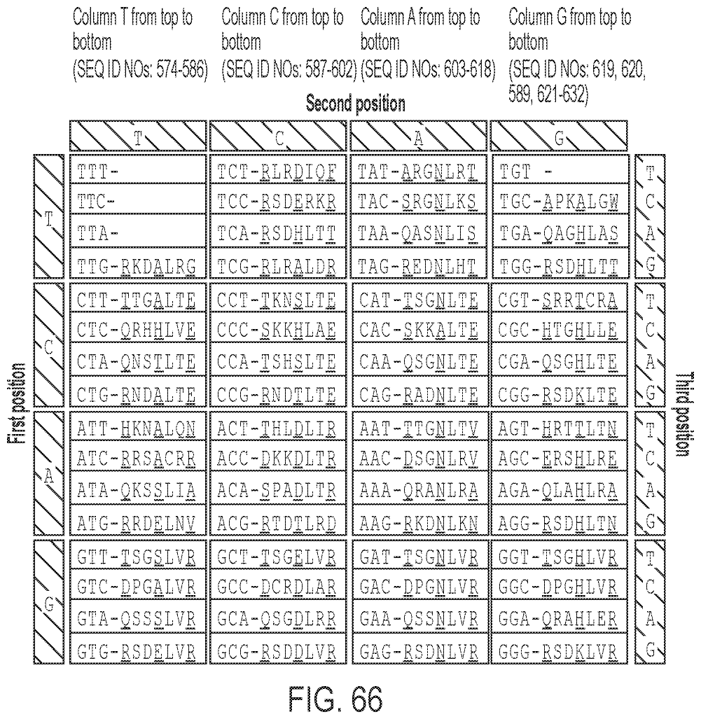

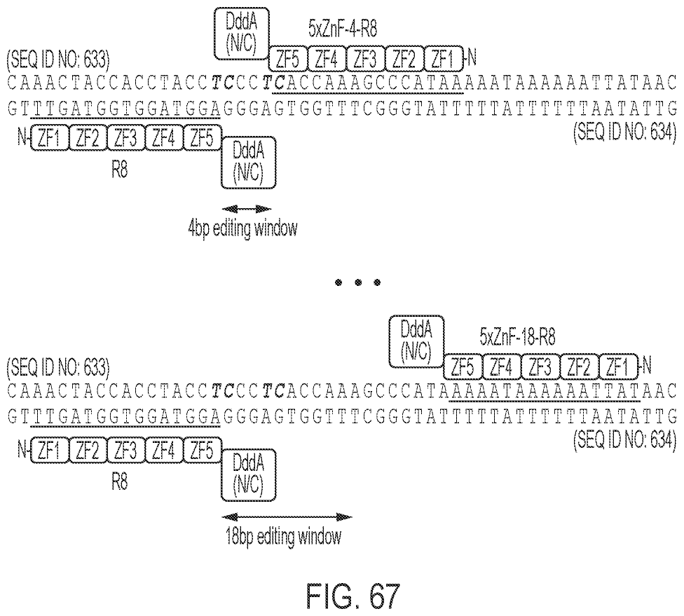

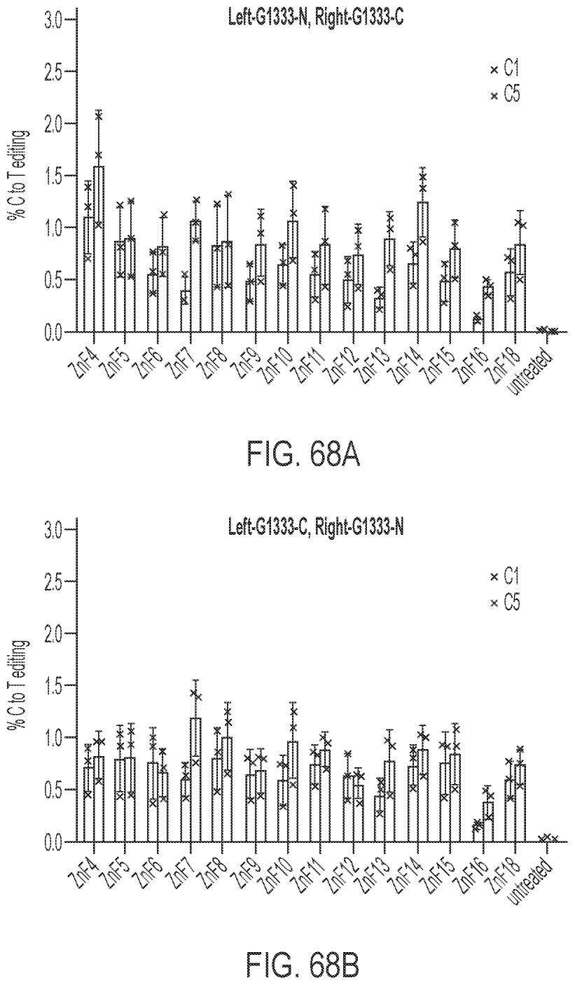

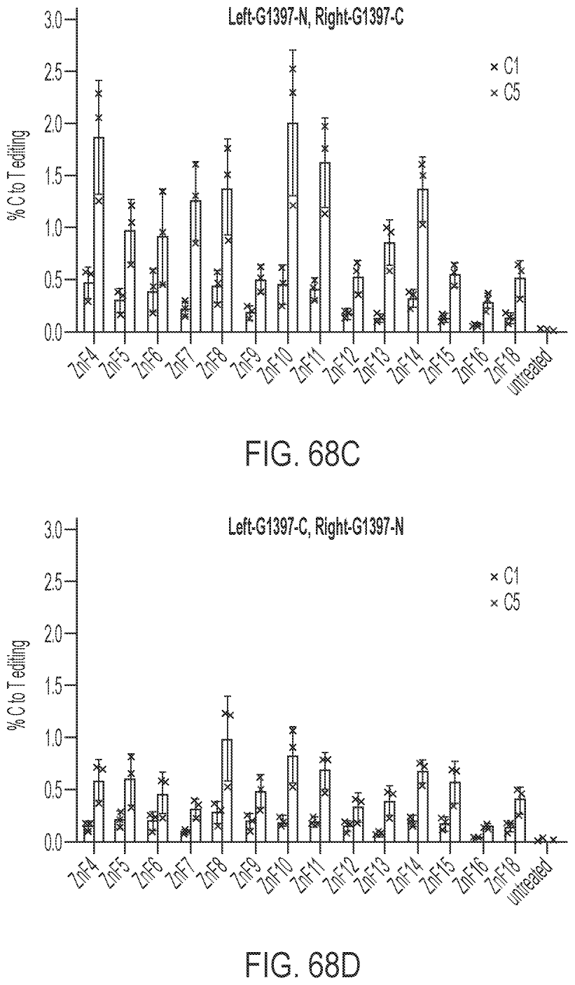

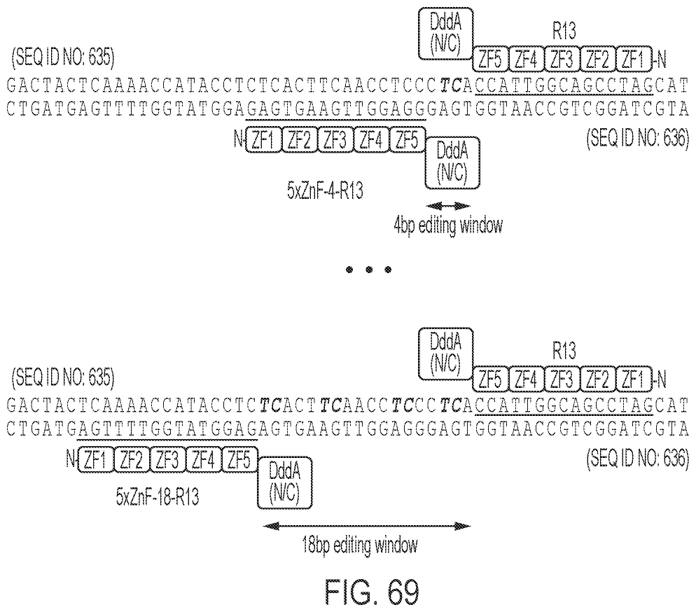

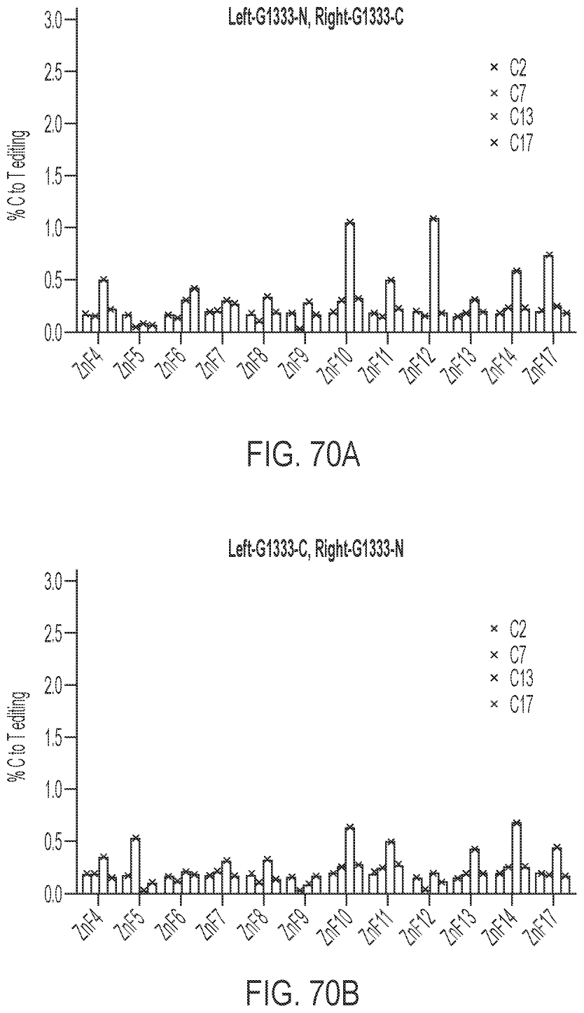

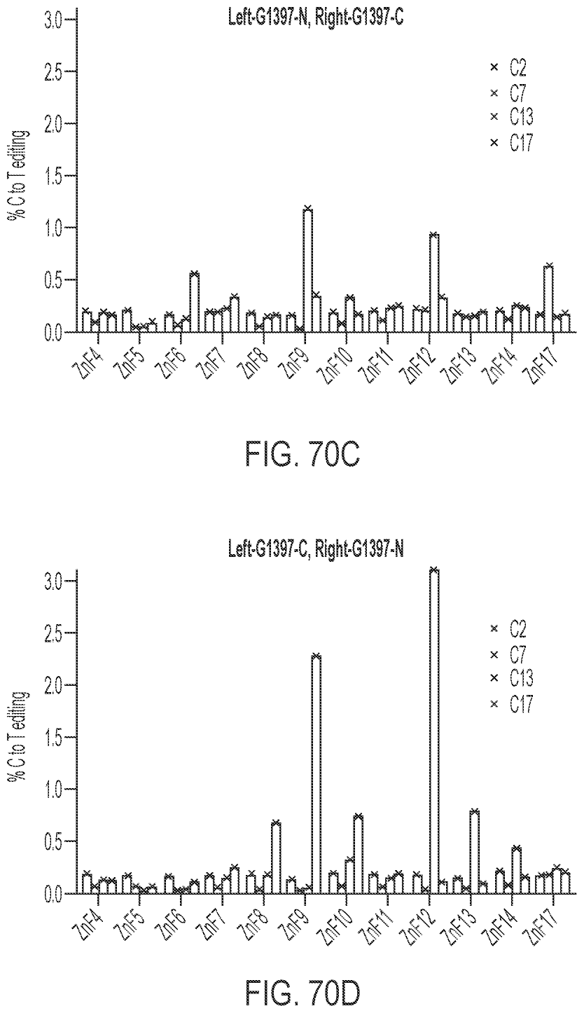

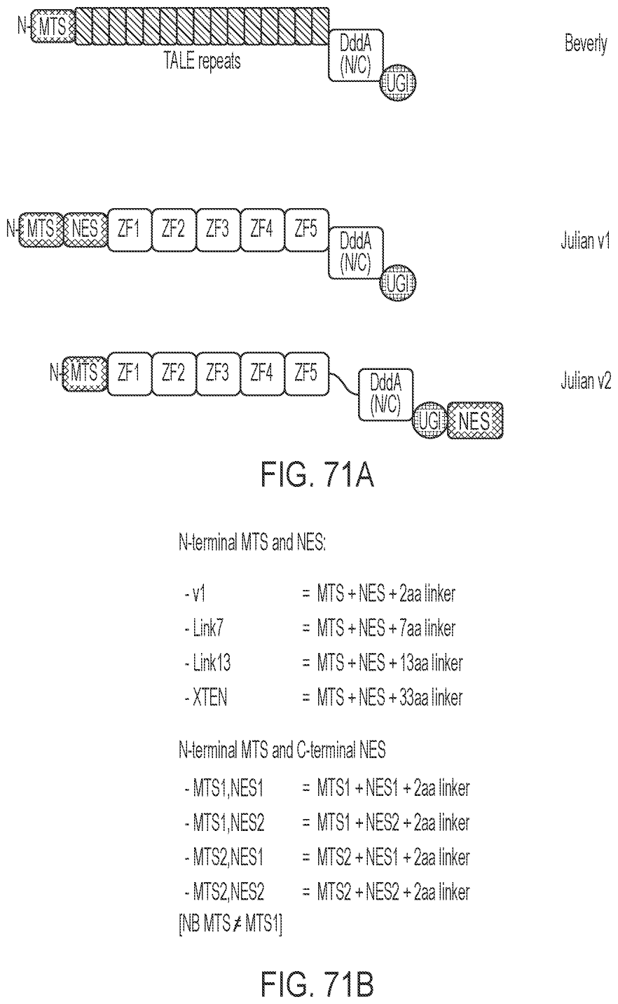

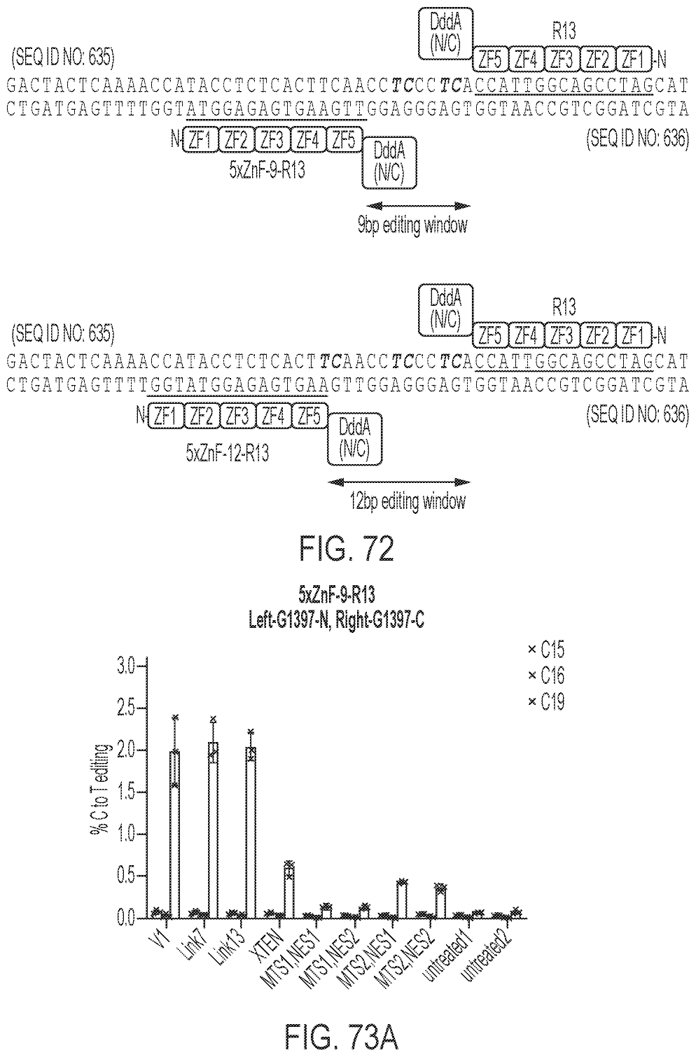

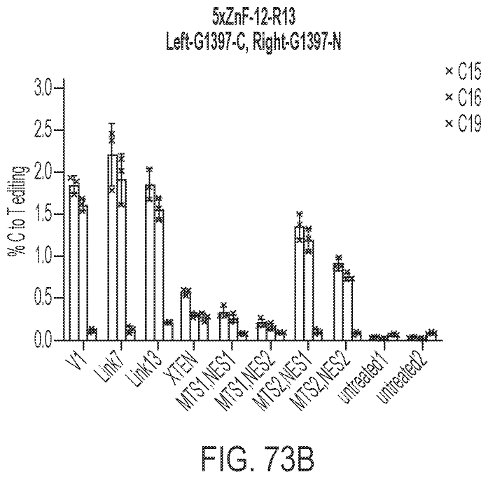

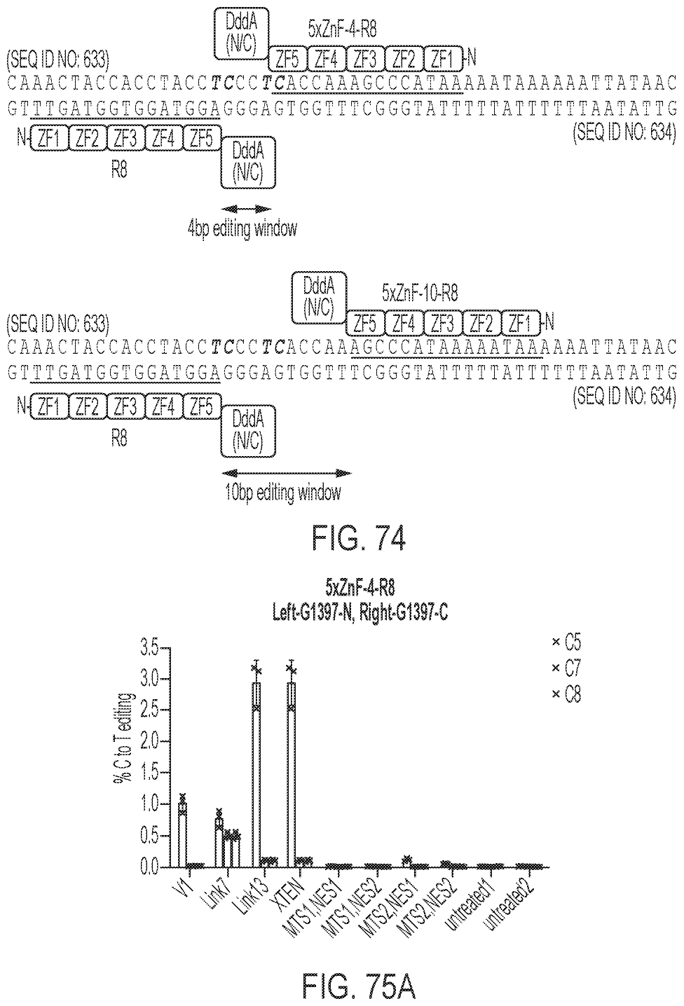

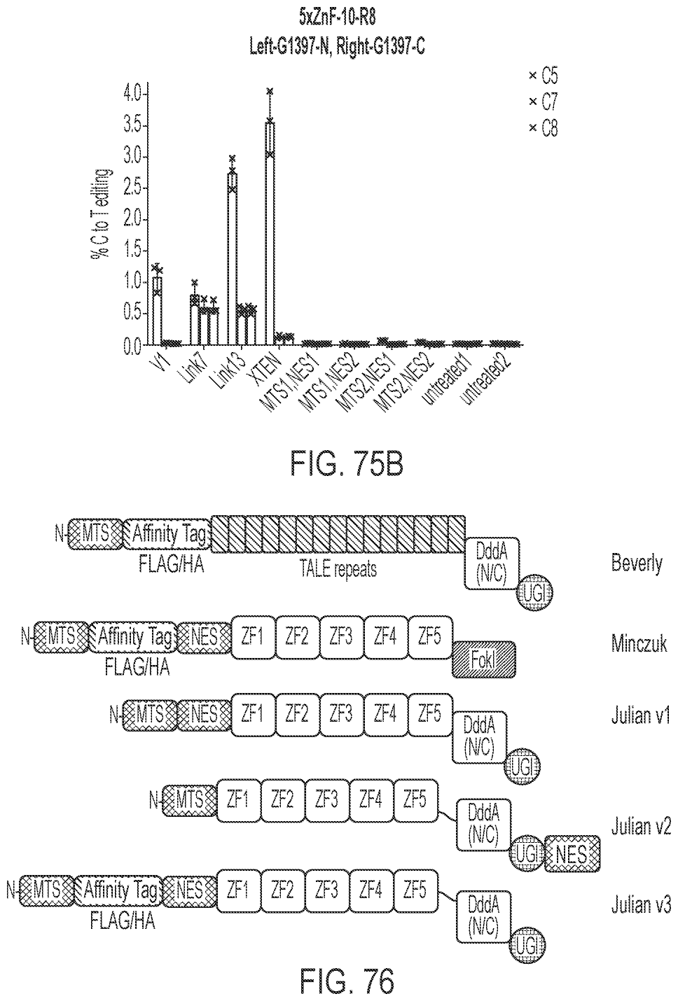

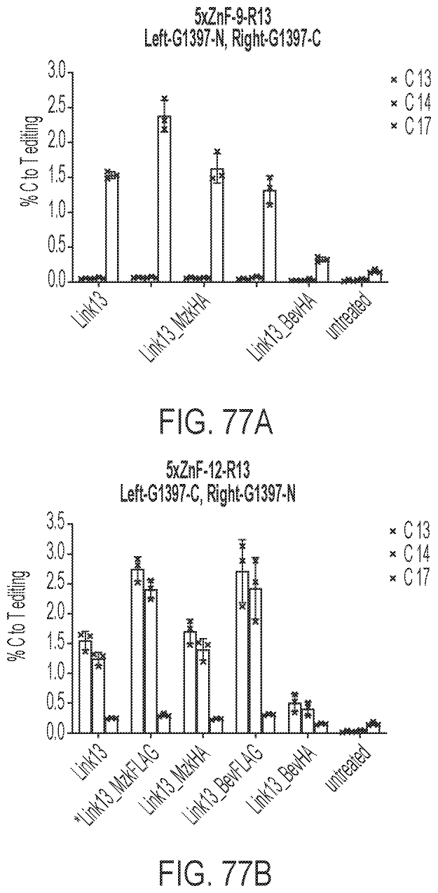

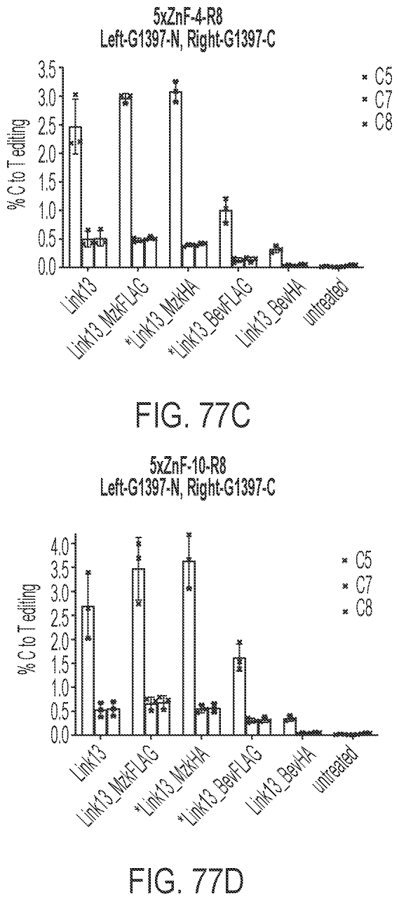

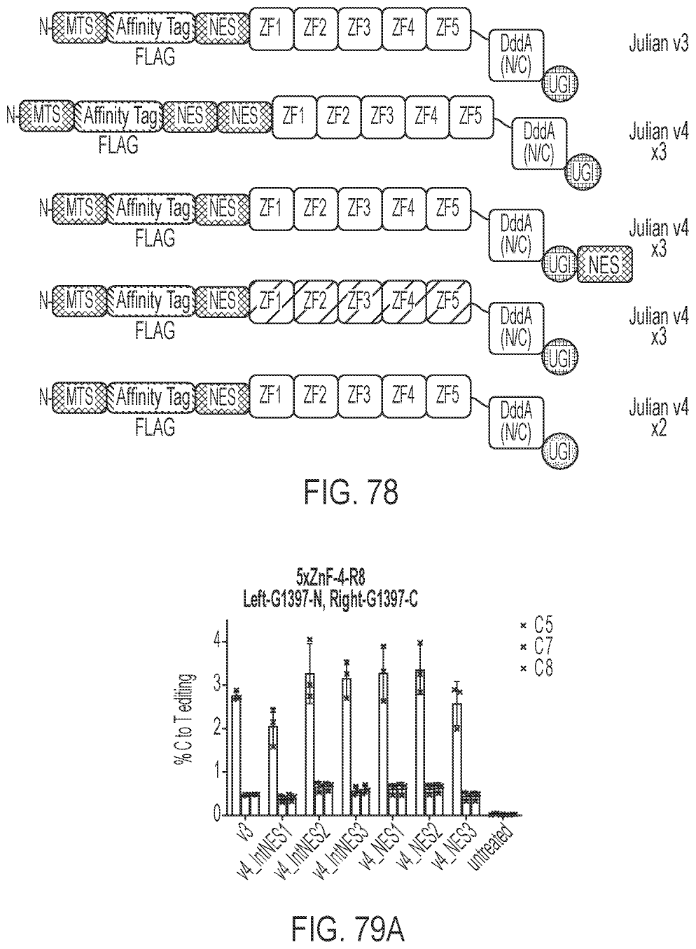

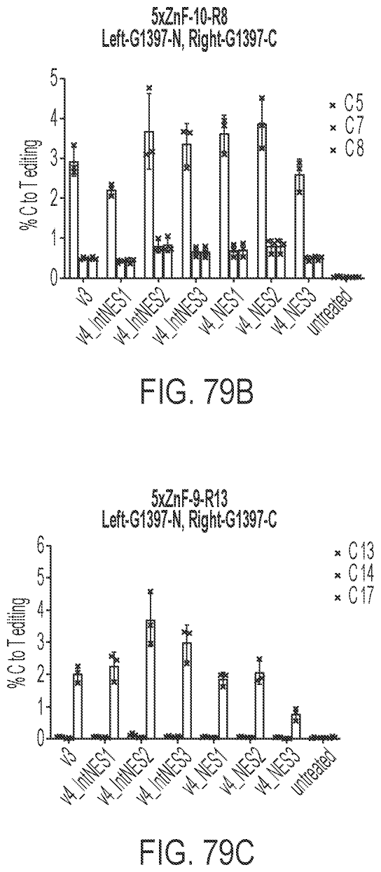

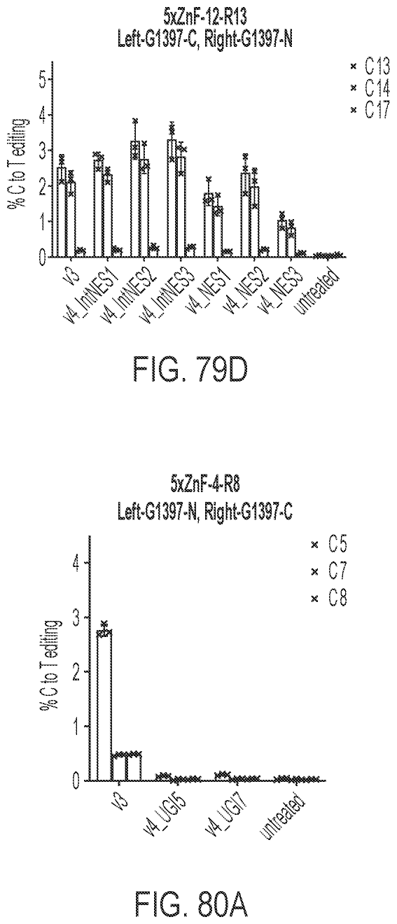

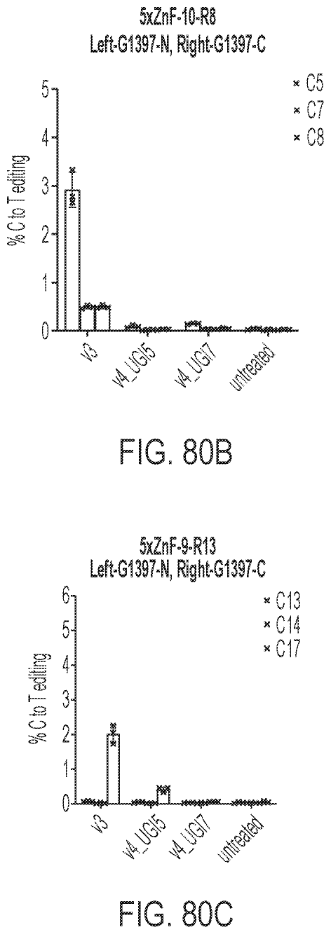

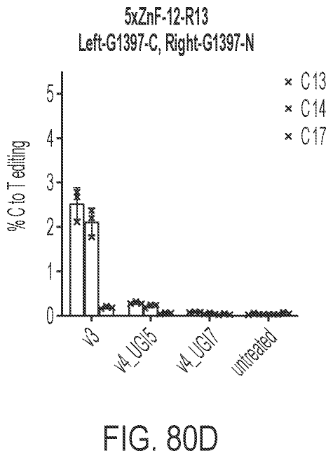

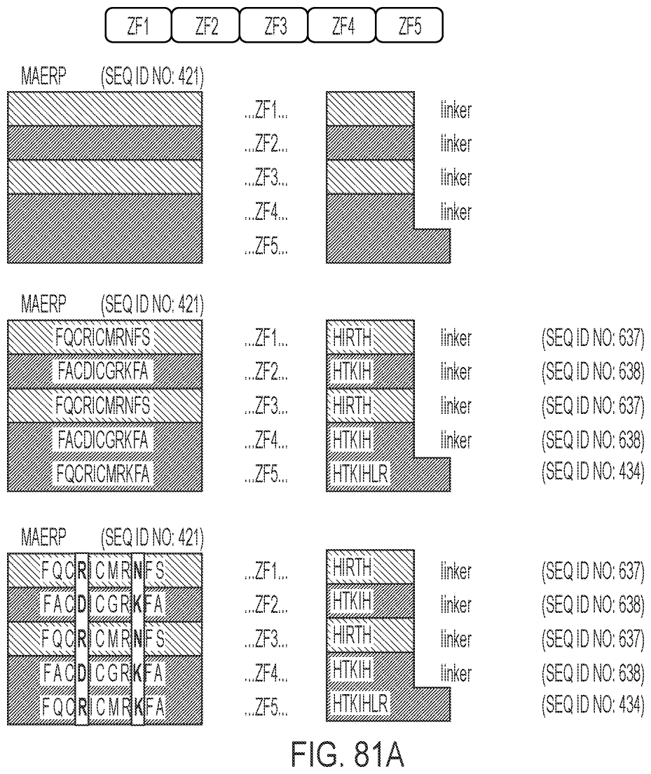

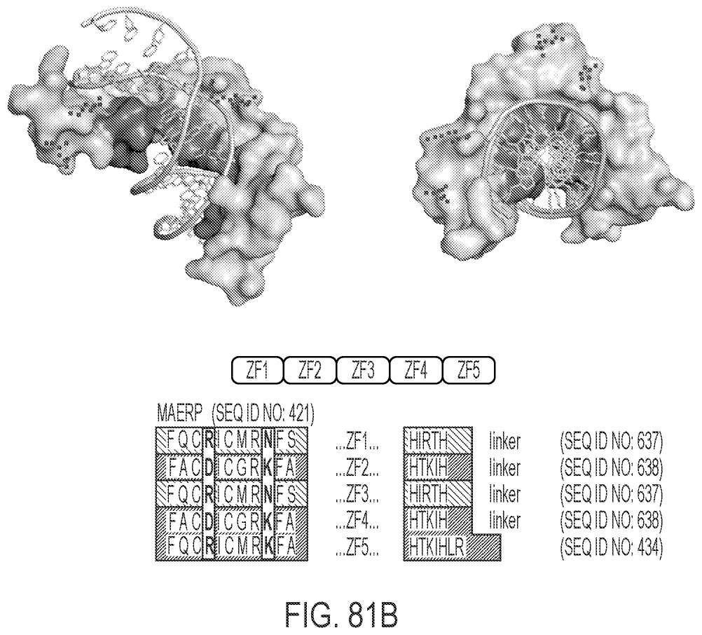

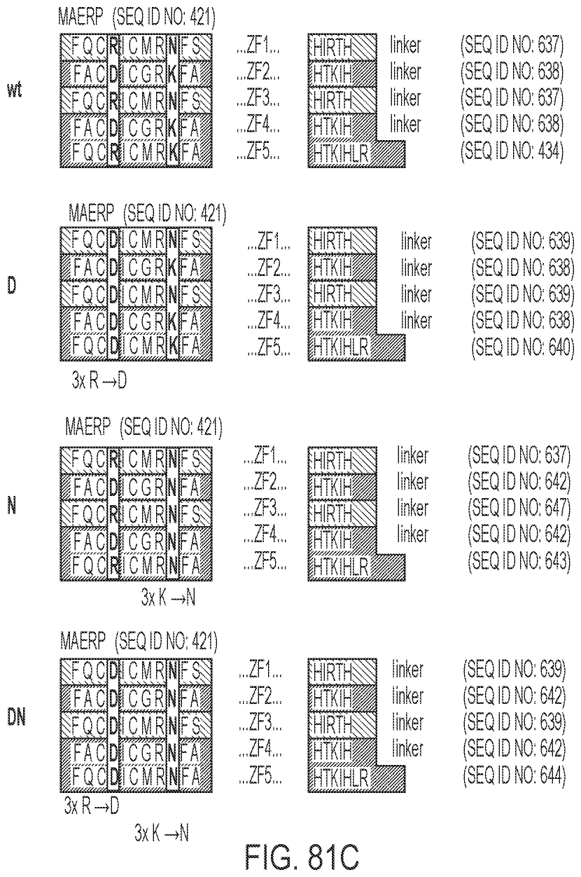

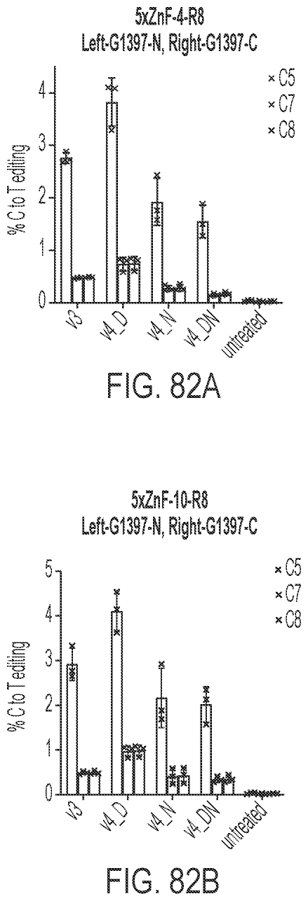

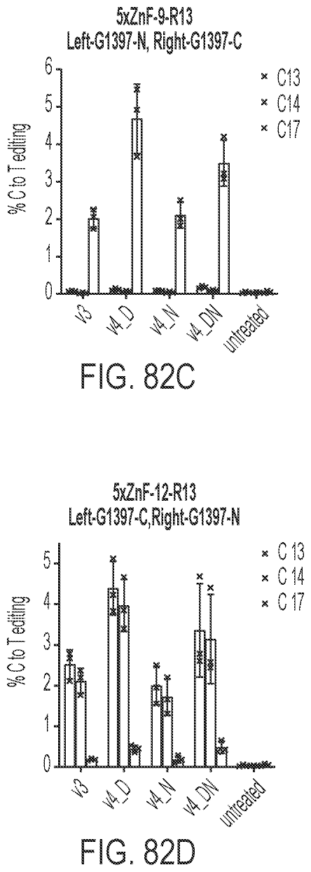

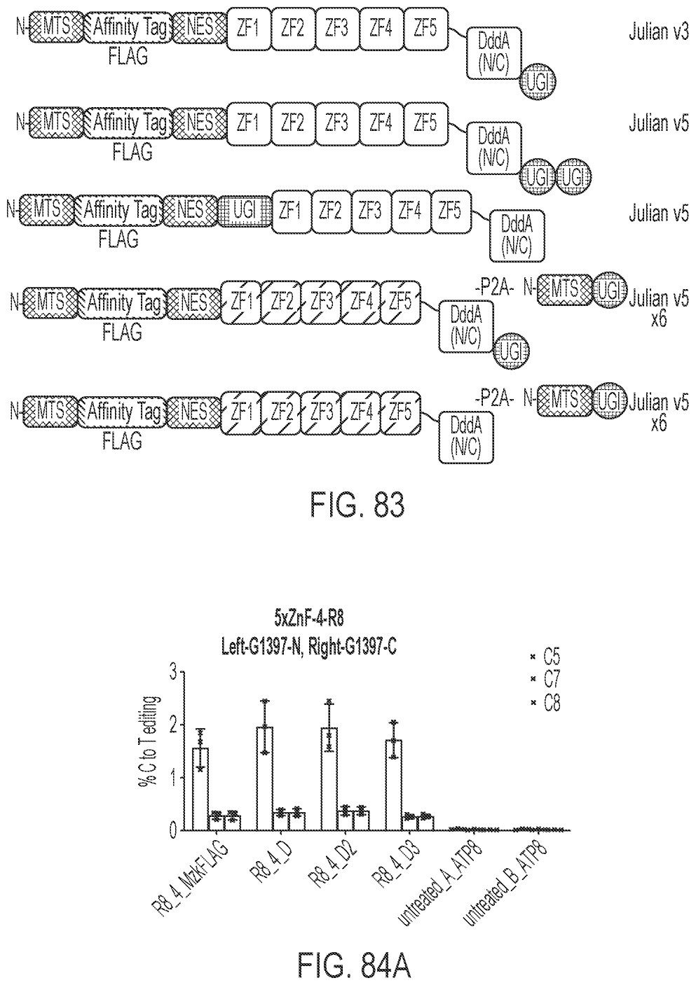

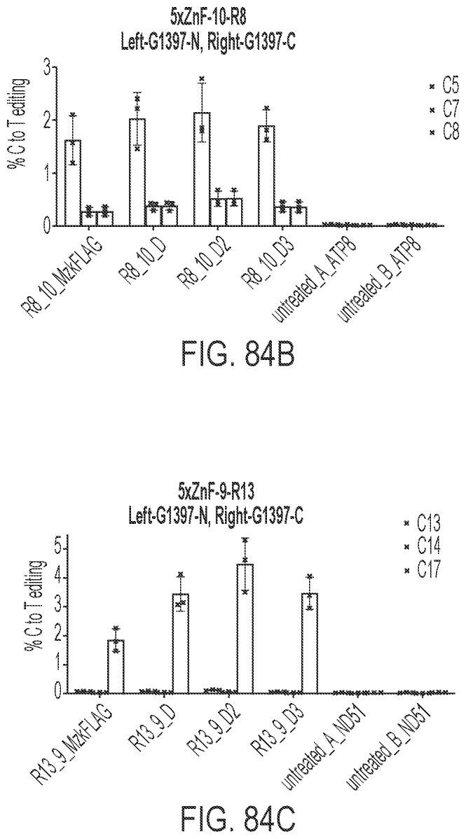

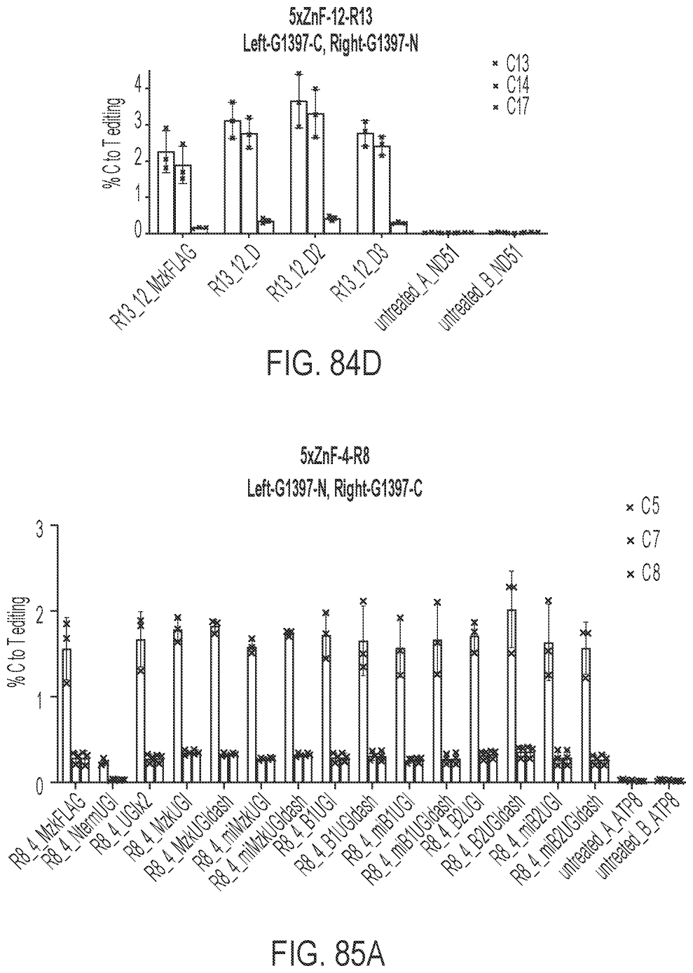

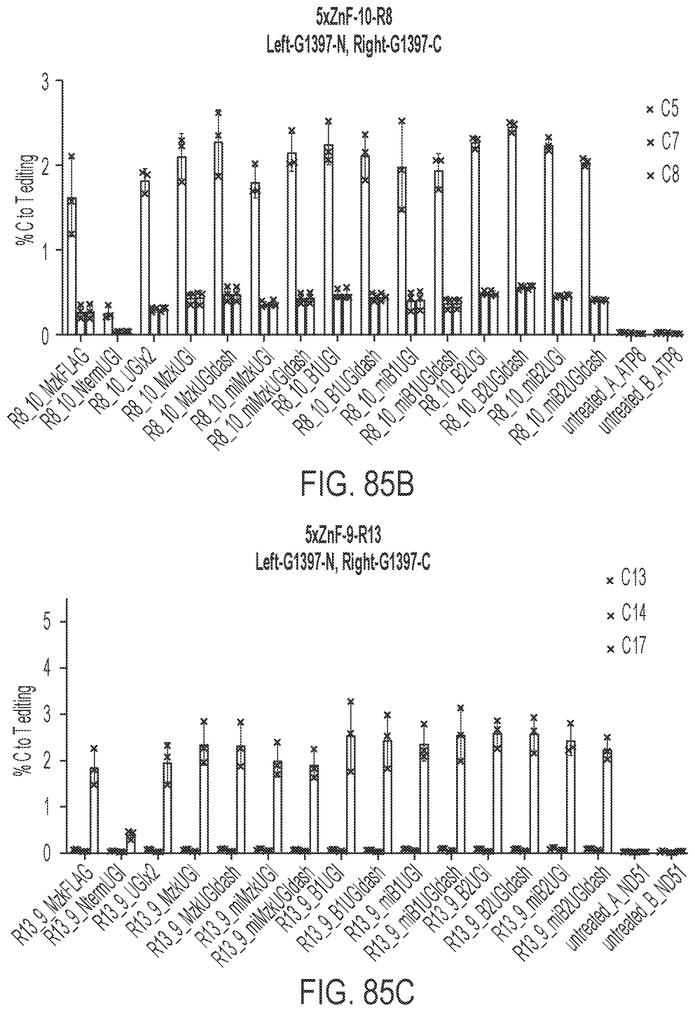

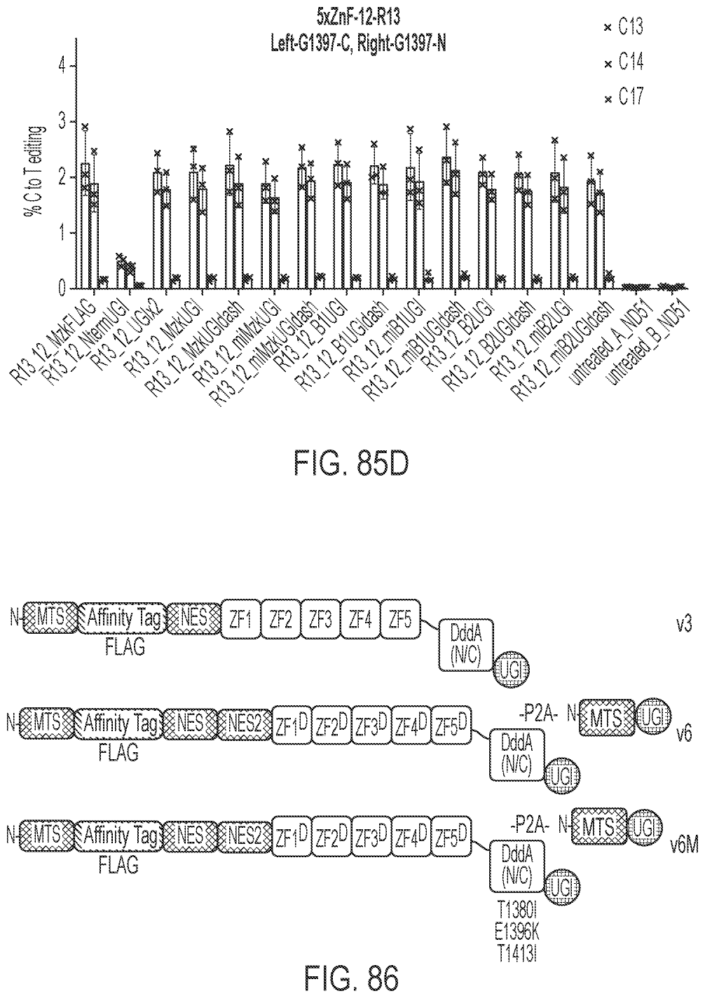

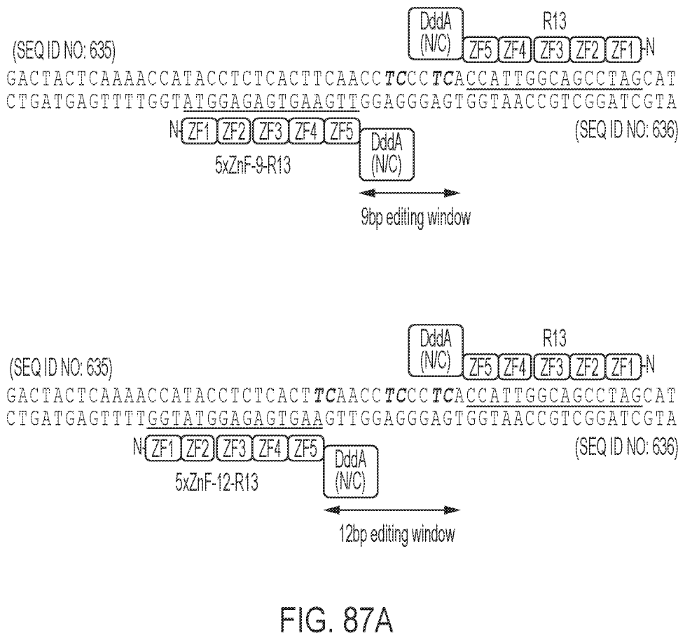

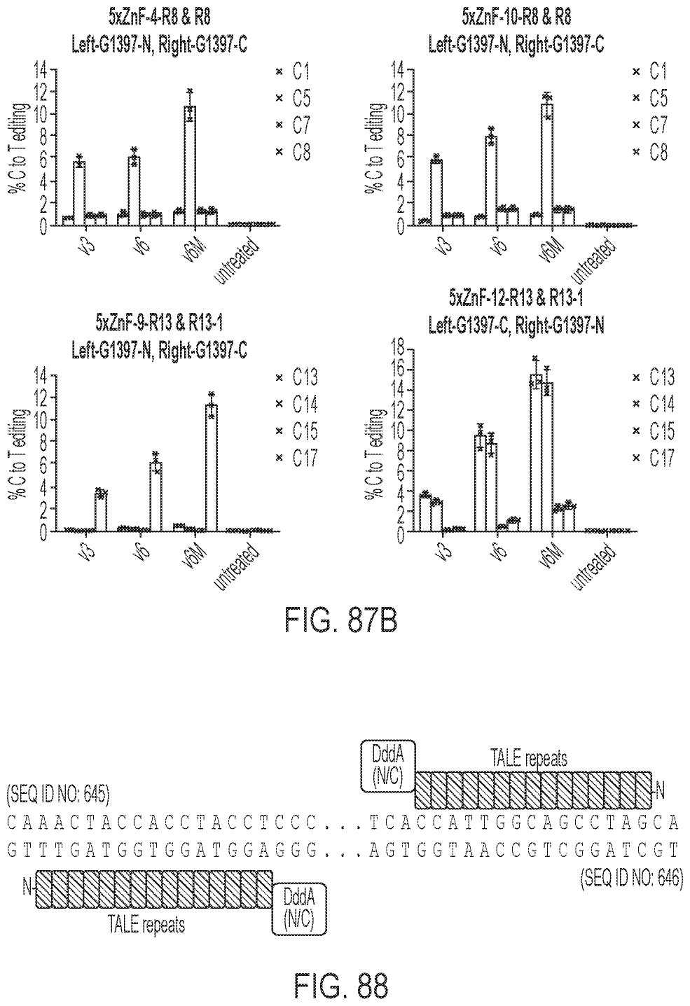

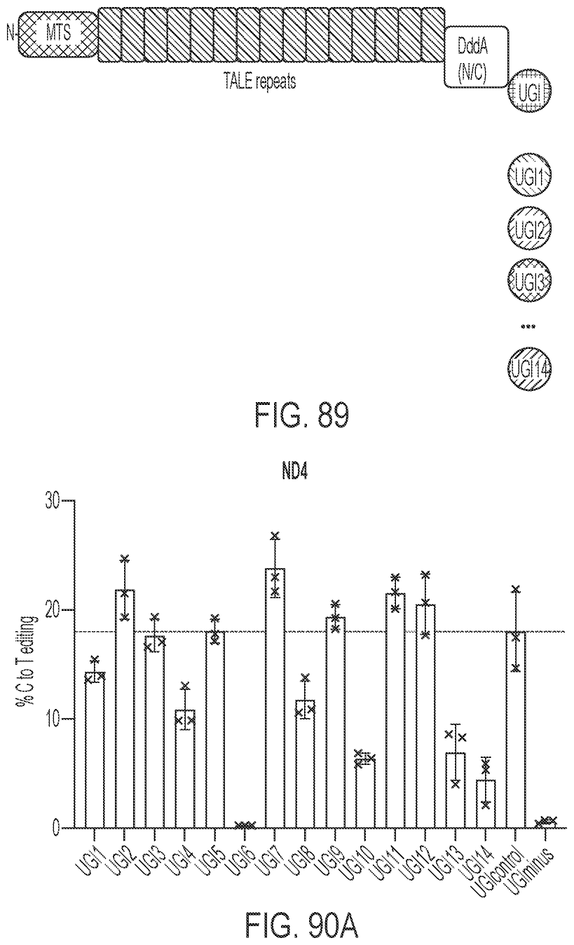

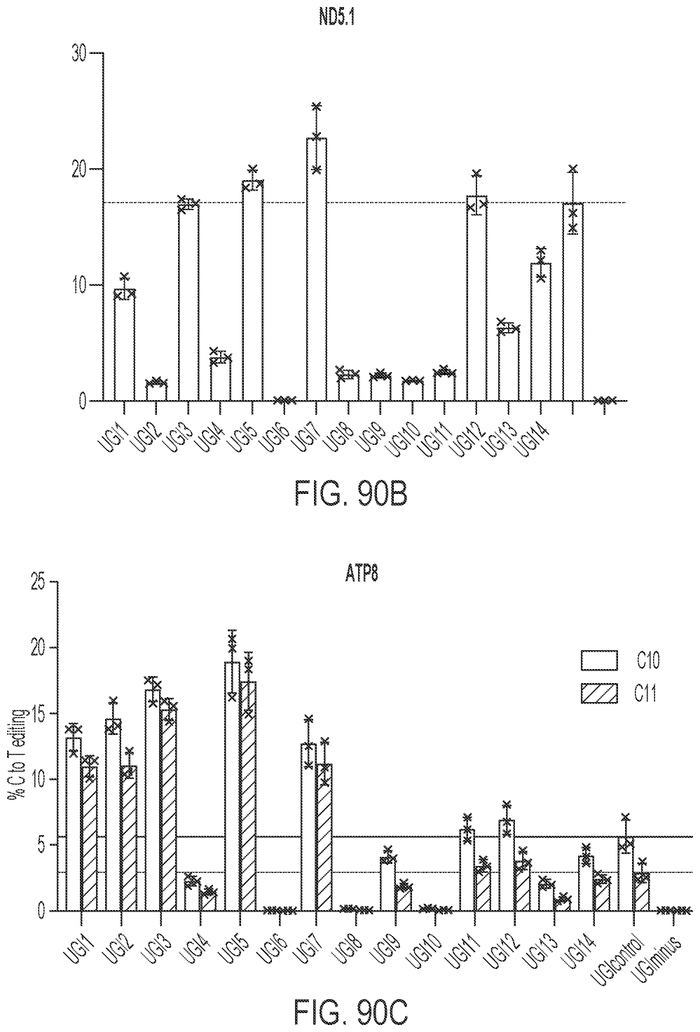

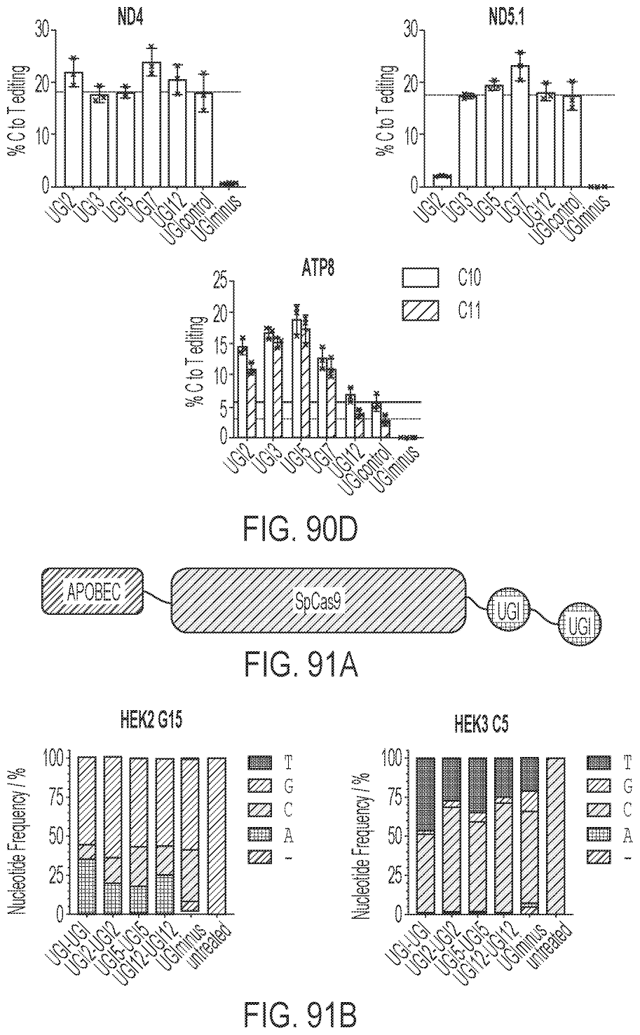

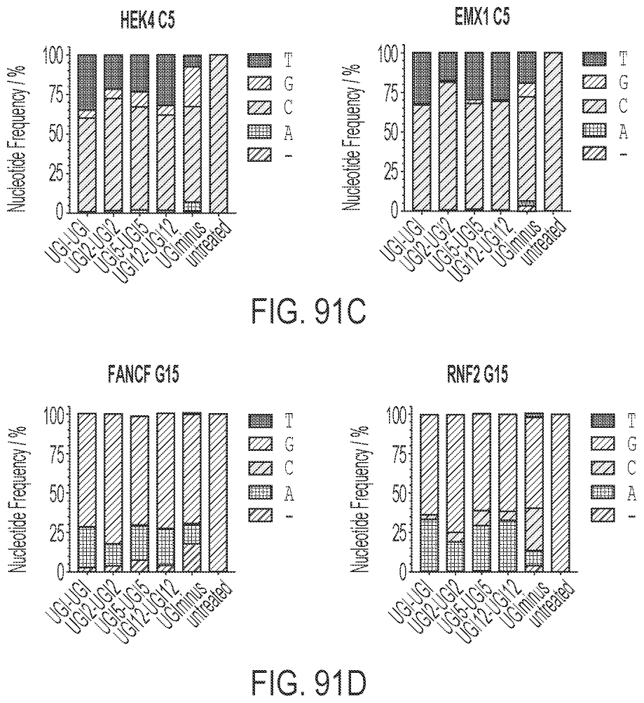

In the context of base editing, all previously described cytidine deaminases utilize single-stranded DNA as a substrate (e.g., the R-loop region of a Cas9-gRNA/dsDNA complex). Base editing in the context of mitochondrial DNA has not heretofore been possible due to the challenges of introducing and/or expressing the gRNA needed for a Cas9-based system into mitochondria. The inventors have recognized for the first time that the catalytic properties of DddA can be leveraged to conduct base editing directly on a double strand DNA substrate by separating the DddA into inactive portions, which when co-localized within a cell will become reconstituted as an active DddA. This avoids or at least minimizes the toxicity associated with delivering and/or expressing a fully active DddA in a cell. For example, a DddA may be divided into two fragments at a “split site,” i.e., a peptide bond between two adjacent residues in the primary structure or sequence of a DddA. The split site may be positioned anywhere along the length of the DddA amino acid sequence, so long as the resulting fragments do not on their own possess a toxic property (which could be a complete or partial deaminase activity). In certain embodiments, the split site is located in a loop region of the DddA protein. In the embodiment shown in FIG. 1A, the arrows depict five possible split sites approximately equally spaced along the length of the DddA protein. The depicted embodiment further shows that the DddA was divided into two fragments at a split site located approximately in the middle of the DddA amino acid sequence. The DddA fragment lying to the left of the the split site may be referred to as the “N-terminal DddA half” and the DddA fragment lying to the right of the split site may be referred to as the “C-terminal DddA half.” FIG. 1A identifies these fragments as “DddA half^{A}” and DddA half^{B},” respectively. Depending on the location of the split site, the N-terminal DddA half and the C-terminal DddA half could be the same size, approximately the same size, or very different sizes.

FIG. 1B depicts a pair of mtDNA base editors each comprising a pDNAbp (pDNAbp A and pDNAbp B) fused to an inactive fragment of DddA (DddA half^{A} and DddA half^{B}). The pDNAbp components bind to their cognate target sites (target site A and target site B) on the mtDNA, thereby localizing the inactive DddA fragments at the target edit/deamination site. Once localized, the DddA activity is restored. It should be noted, that while the pDNAbpA is shown binding to a target site which is physically arranged on the same side of the deamination site as the DddA half^{A}, the DddA half^{A} may be physically arranged so that it approaches the deamination site (e.g., for reconstitution) from any side (e.g., same side, top, opposite side, bottom, or any other angle to the deamination site (e.g., off-axis)) such that it may reconsistute with its DddA half^{B}. Additionally, while the figure shows the pDNAbpA and pDNAbpB binding to target sites on opposite sides of the deamination site, it can be readily envisioned that in view of the aforementinond description regarding orientation, that the two pDNAbp (e.g., A and B) may bind on the same side of the deamination site or opposite sides, provided that the DddA halves may reconstitute and effect deamination at the deamination site. Moreover, while the figure shows the pDNAbpA and pDNAbpB binding to target sites on opposite strands of the DNA duplex, it can be readily envisioned that in view of the aforementinond description regarding orientation, that the two pDNAbp (e.g., A and B) may bind on the same strand of the DNA duplex or opposite strands, provided that the DddA halves may reconstitute and effect deamination at the deamination site. Using these premises, it can readily be envisioned that in some embodiments, the DddA halves are oriented in any position relative to the deamination site such that they effectuate deamination, and further that the pDNAbp to which they are linked may be on the same side or different side of the deamination site, and in some embodiments, such pDNAbp of each of the DddA halves are on the same side of the deamination site, on different sides of the deamination site, are on the same strand of the DNA duplex, or on different strands of the DNA duplex.

FIG. 1C depicts a pair of mtDNA base editors each comprising a mitoTALE (mitoTALE A and mitoTALE B) fused to an inactive fragment of DddA (DddA half^{A} and DddA half^{B}). The mitoTALE components bind to their cognate target sites (target site A and target site B) on the mtDNA, thereby localizing the inactive DddA fragments at the target edit/deamination site. Once localized, the DddA activity is restored. It should be noted, that while the mitoTALEA is shown binding to a target site which is physically arranged on the same side of the deamination site as the DddA half^{A}, the DddA half^{A} may be physically arranged so that it approaches the deamination site (e.g., for reconstitution) from any side (e.g., same side, top, opposite side, bottom, or any other angle to the deamination site (e.g., off-axis)) such that it may reconsistute with its DddA half^{B}. Additionally, while the figure shows the mitoTALEA and mitoTALEB binding to target sites on opposite sides of the deamination site, it can be readily envisioned that in view of the aforementinond description regarding orientation, that the two mitoTALE (e.g., A and B) may bind on the same side of the deamination site or opposite sides, provided that the DddA halves may reconstitute and effect deamination at the deamination site. Moreover, while the figure shows the mitoTALEA and mitoTALEB binding to target sites on opposite strands of the DNA duplex, it can be readily envisioned that in view of the aforementinond description regarding orientation, that the two mitoTALE (e.g., A and B) may bind on the same strand of the DNA duplex or opposite strands, provided that the DddA halves may reconstitute and effect deamination at the deamination site. Using these premises, it can readily be envisioned that in some embodiments, the DddA halves are oriented in any position relative to the deamination site such that they effectuate deamination, and further that the mitoTALE to which they are linked may be on the same side or different side of the deamination site, and in some embodiments, such mitoTALE of each of the DddA halves are on the same side of the deamination site, on different sides of the deamination site, are on the same strand of the DNA duplex, or are on different strands of the DNA duplex.

FIG. 1D depicts a pair of mtDNA base editors each comprising a mitoZFP (mitoZFP A and mitoZFP B) fused to an inactive fragment of DddA (DddA half^{A} and DddA half^{B}). The mitoZFP components bind to their cognate target sites (target site A and target site B) on the mtDNA, thereby localizing the inactive DddA fragments at the target edit/deamination site. Once localized, the DddA activity is restored. It should be noted, that while the ZFPA is shown binding to a target site which is physically arranged on the same side of the deamination site as the DddA half^{A}, the DddA half^{A} may be physically arranged so that it approaches the deamination site (e.g., for reconstitution) from any side (e.g., same side, top, opposite side, bottom, or any other angle to the deamination site (e.g., off-axis)) such that it may reconsistute with its DddA half^{B}. Additionally, while the figure shows the ZFPA and ZFPB binding to target sites on opposite sides of the deamination site, it can be readily envisioned that in view of the aforementinond description regarding orientation, that the two ZFP (e.g., A and B) may bind on the same side of the deamination site or opposite sides, provided that the DddA halves may reconstitute and effect deamination at the deamination site. Moreover, while the figure shows the ZFPA and ZFPB binding to target sites on opposite strands of the DNA duplex, it can be readily envisioned that in view of the aforementinond description regarding orientation, that the two ZFP (e.g., A and B) may bind on the same strand of the DNA duplex or opposite strands, provided that the DddA halves may reconstitute and effect deamination at the deamination site. Using these premises, it can readily be envisioned that in some embodiments, the DddA halves are oriented in any position relative to the deamination site such that they effectuate deamination, and further that the ZFP to which they are linked may be on the same side or different side of the deamination site, and in some embodiments, such ZFP of each of the DddA halves are on the same side of the deamination site, on different sides of the deamination site, are on the same strand of the DNA duplex, or are on different strands of the DNA duplex.

FIG. 1E depicts a pair of mtDNA base editors each comprising a Cas9 (Cas9 A and Cas9 B) fused to an inactive fragment of DddA (DddA half^{A} and DddA half^{B}). The Cas9 components bind to their cognate target sites (target site A and target site B) on the mtDNA as programmed by their respective guide RNAs, thereby localizing the inactive DddA fragments at the target edit/deamination site. Once localized, the DddA activity is restored. It should be noted, that while the Cas9A is shown binding to a target site which is physically arranged on the same side of the deamination site as the DddA half^{A}, the DddA half^{A} may be physically arranged so that it approaches the deamination site (e.g., for reconstitution) from any side (e.g., same side, top, opposite side, bottom, or any other angle to the deamination site (e.g., off-axis)) such that it may reconsistute with its DddA half^{B}. Additionally, while the figure shows the Cas9A and Cas9B binding to target sites on opposite sides of the deamination site, it can be readily envisioned that in view of the aforementinond description regarding orientation, that the two Cas9 (e.g., A and B) may bind on the same side of the deamination site or opposite sides, provided that the DddA halves may reconstitute and effect deamination at the deamination site. Moreover, while the figure shows the Cas9A and Cas9B binding to target sites on opposite strands of the DNA duplex, it can be readily envisioned that in view of the aforementinond description regarding orientation, that the two Cas9 (e.g., A and B) may bind on the same strand of the DNA duplex or opposite strands, provided that the DddA halves may reconstitute and effect deamination at the deamination site. Using these premises, it can readily be envisioned that in some embodiments, the DddA halves are oriented in any position relative to the deamination site such that they effectuate deamination, and further that the Cas9 to which they are linked may be on the same side or different side of the deamination site, and in some embodiments, such Cas9 of each of the DddA halves are on the same side of the deamination site, on different sides of the deamination site, are on the same strand of the DNA duplex, or are on different strands of the DNA duplex.

FIG. 1F. depicts a variety of architectural embodiments envisioned for the constructs described in any of FIGS. 1A to 1E. These architectural embodiments are not intended to limit the present disclosure as other architectures are also feasible and are contemplated by this disclosure. Embodiment (a) depicts a first fusion protein comprising a pDNAbp (arbitrarily labeled pDNAbp A) fused to a DddA half domain (arbitrarily labeled DddA half A) which binds to a first target site on a strand of a double-stranded DNA molecule (e.g., a miDNA). The first target site is arbitrarily labeled “target site A.” This embodiment also depicts a second fusion protein comprising a second pDNAbp (i.e., pDNAbp B) fused through a linker to a second DddA half (i.e., DddA half B). The second fusion protein is shown binding to a second target site on the opposite strand of DNA as the first target site. The DddA half A and DddA half B associate at the deamination site (“*”) to form a functional DddA which then proceeds to deaminate the deamination site. As illustrated by architerctural embodiments in (a) through (e), the target sites are located on opposite strands of the DNA, with the pDNAbps binding to opposite strands. Embodiments (f) through (k), however, show that the target sites may be located on the same strand, with the pDNAbps binding to the same strands. In some embodiments, such as in (f) through (i), the target sites to which the pDNAbps bind are located on the same strand containing the target deamination site (“*”). In other embodiments, as depicted in (i) through (k), the target sites to which the pDNAbps bind are located on the strand opposite the strand containing the target deamination site (“*”). In addition, the fusion proteins can be arranged in any suitable linear order of domains, including N-[dDNAbp]-[linker]-[DddA half]-C and N-[DddA half]-[linker]-[dDNAbp]-C. Still further, the fusion proteins may be configured such that the DddA halves (e.g., DddA half A and DddA half B) associate near or adjacent the deamination target site, such as in same-side association near the deamination site in (d) or (f), or opposite-side association opposite the deamination site in (e) and (i), or combinations of these configurations, as in (a), (b), (c), (g), (h), (j), (k), or (1) through (q). In addition, the linker may fuse the DddA domain to either side of the pDNAbp, as shown in the variations of (1) through (q), or combinations of these embodiments. In addition, the DddA halves may associate with one another on either side of the target deamination site (e.g., compare embodiment (r) versus any of the embodiments of (a) through (q). The disclosure is not limited to the embodiments depicted.

FIGS. 2A-2B show DddA toxin is a double-stranded DNA cytidine deaminase toxin. FIG. 2A: Top, In vitro cytidine deamination assay using single-stranded DNA (left) or double-stranded (right) 6-carboxyfluorescein-labelled DNA substrate. DddA has a stronger preference for deaminating cytidines in the 5′-TC context compared to cytidines in the 5′-GC context. Middle, Viability of E. coli populations expressing active DddA (ddd), catalytically inactive DddA (dddE98A), induced after 4 h. Viability of DddA-expressing E. coli decreases drastically while the viability of populations containing inactive DddA remain comparable to the non-induced control. Number of SNPs from the indicated nucleotide classifications observed in E. coli following intoxication with DddA or inactive DddA. DddA-expressing E. coli contains >100-fold C·G-to-T·A transitions compared to strains expressing inactive DddA. Bottom, Co-crystal structure of DddA bound to immunity protein DddI. FIG. 2B: Differences between a ssDNA cytidine deaminase editor and a hypothetical dsDNA cytidine deaminase base editor. The previously published rAPOBEC1-Cas9 nickase-UGI fusion is an example of a ssDNA cytidine deaminase. DddA-derived cytosine base editor (DdCBE) is an example of a dsDNA cytidine deaminase editor

FIG. 3. shows screening for split sites in DddA to overcome the toxicity of full-length DddA. DddA was split at 12 different sites as listed in the table. For each split, the N-terminal (DddA-N) and C-terminal (DddA-C) halves were each fused to a dCas9-2×UGI protein to form DddA-N-dCas9-2×UGI and DddA-C-dCas9-2×UGI, respectively. Both halves were plasmid transfected into HEK293T cells. Genomic DNA was harvested after 3 days for high-throughput DNA sequencing. Active split-DddA fusions were identified based on the percentage of C·G-to-T·A conversion at target cytidines within the spacing region flanked by the two dCas9 proteins.

FIG. 4 shows splitting DddA at G148 and G84 resulted in two inactive halves that reconstitute activity when co-localized on DNA in HEK293T cells. Nucleotide percentage summary plots showing the percentage of nucleotides at each position of the target spacing region. The appearance of “*” within the C-containing positions indicate C-to-T conversion while the appearance of “\hat{ }” within the G-containing positions indicate G-to-A conversion.

FIG. 5 shows inactive DddA-N and DddA-C halves fused to orthogonal Cas9 proteins reassemble into an active cytidine deaminase. The initial screen was performed with two identical dCas9 proteins, thus precluding control of DddA fusion orientation. In this screen, two fusion orientations are possible for a given DddA split. The aureus-N orientation comprises of DddA-C-dCas9-2×UGI and DddA-N-SaKKH-Cas9(D10A)-1×UGI. The aureus-C orientation comprises of DddA-N-dCas9-2×UGI and DddA-C-SaKKH-Cas9(D10A)-1×UGI. The nucleotide percentage summary plots shows C·G-to-T·A conversion for the G148 split in the DddA-N-dCas9-2×UGI and DddA-C-SaKKH-Cas9(D10A)-1×UGI orientation.

FIG. 6 shows the architecture of DdCBE. DdCBE comprises of a left monomer and right monomer. The architecture of each monomer of a mitoTALE-split-DddAtox pair (in N- to C-terminus order): an MTS, a TALE array, a 2-amino acid linker, a DddAtox half from the G1333 or G1397 split, and one or two UGI proteins. Final optimization studies indicate higher editing efficiencies with one copy of UGI protein. The TALE proteins of the indicated DdCBE binds to the human MT-ND6 gene.

FIG. 7 shows the architecture of DdCBE. DdCBE comprises of a left monomer and right monomer. The architecture of each monomer of a mitoTALE-split-DddAₜₒₓ pair (in N- to C-terminus order): an MTS, a TALE array, a 2-amino acid linker, a DddAₜₒₓ half from the G1333 or G1397 split, and one or two UGI proteins. Final optimization studies indicate higher editing efficiencies with one copy of UGI protein. The TALE proteins of the indicated DdCBE binds to the human MT-ND6 gene.

FIGS. 8A-8B show TALE-DddA constructs. FIG. 8A shows 2×-UGI TALE-split DddA and UGI-free TALE-split halves expresses well in HEK293T cells i. TC31 and TC32 are FokI-based TALENs that target nuclear CCR5 and were included as positive controls. Mito20: SOD2 MTS-3×HA-left TALE m.14459A TALE-2aa¬-G1333 DddA-N; Mito20a: SOD2 MTS-3×HA-2×UGI-left TALE m.14459A TALE-2aa¬-G1333 DddA-N; mito26: COX8a MTS-3×FLAG-right TALE m.14459A TALE(Nt-αN)-2aa-G1333 DddA-C; mito26a: COX8a MTS-3×FLAG-2×UGI-right TALE m.14459A TALE(Nt-αN)-2aa-G1333 DddA-C; mito30: COX8a MTS-3×FLAG-right TALE m.14459A TALE(Nt-βN)-2aa-G1333 DddA-C; mito30a: COX8a MTS-3×FLAG-2×UGI-right TALE m.14459A TALE(Nt-βN)-2aa-G1333 DddA-C. FIG. 8B shows the architectures of TALE-DddA constructs listed in FIG. 8A.

FIGS. 9A-9B show TALE-G1397 split DddA fusions. FIG. 9A shows the architectures of TALE-G1397 split DddA fusions. DddA was split at G1397. The N-terminus half was fused to the left TALE, and the C-terminus half is fused to the Right TALE. TALE sequences target the human MT-ND6 gene. The N-terminal domain of the right TALE is modified (Nt-αN) to recognize non-T nucleotides at the 5′ position immediately after the first nucleotide of the TALE binding sequence. FIG. 9B shows TALE-G1397 split DddA fusions edits the MT-ND6 gene. Genomic context MT-ND6. TALE binding sites are annotated in lime green. Nucleotide percentage summaries of each positions within the target spacing region is shown. C-to-T conversion is shown by the appearance of “*”.

FIG. 10 shows N-terminal UGI fusions abrogate editing activity. Mito 24a and Mito 28a each contains 2 copies of UGI protein fused to the N-terminus of the TALE-split DddA fusion. Nucleotide percentage summaries of HEK193T cells treated with Mito 24a and Mito 28a show the absence of editing at the target C (arrow).

FIG. 11 shows N-terminal UGI fusions do not localize into the mitochondria. Fluorescence imaging of HA- and FLAG-tagged halves of mitoTALE-DddAtox and UGI-mitoTALE-DddAtox-UGI pairs in HeLa cells 24 h after plasmid transfection. Mitochondrial localization was followed using Mitotracker. Non-UGI containing fusions (mito 24 and mito 28) localized to the mitochondria while N-terminal UGI fusions (mito 24a and mito 28a) remain diffused throughout the cytoplasm.

FIGS. 12A-12B show adding UGI to the C-terminus of mitoTALE-DddA fusions improves editing efficiencies. FIG. 12A: ND6 editing efficiencies from fusions containing 1×- or 2×-UGI proteins at the N- or C-terminus 3 days post-transfection are shown. Refer to FIG. 13 for the architectures of each construct. Schematic representation of the mitoTALE-split DddA fusions is shown. FIG. 12B: Nucleotide percentage summaries at MT-ND6. Fusions containing one copy of UGI protein (C-terminus 1×UGI) results in a slightly higher editing that fusions containing two copies of UGI protein (C-terminus 2×UGI).

FIG. 13 is a summary of architecture, editing efficiency and mitochondria localization of the respective constructs listed in FIG. 12A. N.D., not detectable.

FIG. 14 shows alternative mitochondria targeting signal (MTS) sequences do not boost editing efficiency. The original MTS sequences used were COX8a and SOD2 MTS. Tandem fusions of a maize-derived MTS (zmLOC100282174) to COX8a and SOD2 do not improve editing significantly. BPNLS, bipartite nuclear localization signal.

FIG. 15 is a schematic representation of mitoTALE-split DddA fusion.

FIG. 16 Shows DdCBE editing increases with duration of base editor treatment. MT-ND6 editing efficiencies of HEK293T are shown for the listed constructs. Cells were harvested 3-days or 6-days post-transfection C′-2×UGT and C′-1×UGT are mitoTALE-split DddA fusions that contain 2 copies or 1 copy of UGI appended to the C-terminus, respectively. BPNLS, bipartite nuclear localization signal

FIG. 17 shows DdCBE-edited cells maintain mtDNA copy numbers. mtDNA levels of MT-ND6-edited cells were measured by quantitative PCR relative to untreated cells. Cells that were treated with listed variants of base editors had similar relative mtDNA levels to edited cells, suggesting that DdCBE editing does not impact mtDNA integrity.

FIG. 18 is a schematic of ND5.1-DdCBE. ND5.1-DdCBE was designed to target the wildtype MT-ND5 gene. TALE binding sites are underlined in red; Possible cytidine substrate are in magenta. The Right-G1397-C+Left-G1397-N orientation selectively target C10 within the target spacing region for editing.

FIG. 19 shows ND5.1-DdCBE edits MT-ND5 efficiently in HEK293T cells. MT-ND5 editing efficiencies are shown for the different DdCBE orientations. mitoTALE-split DddA-UGI fusions containing a 2 amino acid- or 16 amino acid-linker gave similar editing efficiencies.

FIG. 20 shows ND5.1-edited cells maintain mtDNA copy numbers. mtDNA levels of MT-ND6-edited cells were measured by quantitative PCR (qPCR) relative to untreated cells. mtDNA levels were normalized to beta-actin. E, efficiency of qPCR.

FIG. 21 is a schematic of ND5.2-DdCBE. TALE binding sites are underlined; Possible cytidine substrate are noted by “*”. The Right-G1397-N+Left-G1397-C orientation selectively target C11 and C12 within the target spacing region for editing.

FIG. 22 shows ND5.2-DdCBE edits MT-ND5 efficiently in HEK293T cells without affecting mtDNA copy numbers. MT-ND5.2 editing efficiencies are shown for the different DdCBE orientations 3 days post-transfection. mtDNA levels of MT-ND6-edited cells were measured by quantitative PCR (qPCR) relative to untreated cells. mtDNA levels were normalized to beta-actin. E, efficiency of qPCR

FIGS. 23A-23I show that DddA is a double-stranded DNA cytidine deaminase that mediates T6SS-dependent T6SS-interbacterial antagonisms. FIG. 23A is a schematic depicting domains of full-length DddA. The C-terminal toxin domain (tox) used in later experiments is shown in purple. FIG. 23B shows the competitiveness of the indicated donor B. cenocepacia strains (D) toward the B. cenocepacia ΔdddA ΔdddA₁ recipient strain (R), which is sensitized to DddA intoxication. Values and error bars represent the mean±s.d. of n=2 technical replicates indicative of at least six biological replicates. *P<0.05 by Student's unpaired two-tailed t-test. FIG. 23C shows the viability of E. coli populations expressing the indicated deaminases. The arrow indicates time of induction of the specified genes. Values and error bars represent the mean±s.d. of n=2 technical replicates indicative of at least three biological replicates. FIG. 23D is a schematic of the crystal structure of DddAₜₒₓ(ribbon) complexed with DddAᵢ(space filling). The DddAtox-associated Zn²⁺ ion is shown and residues critical to Zn²⁺ coordination (H1345) and catalysis (E1347) are indicated. FIG. 23E shows the structural alignment of DddAₜₒₓ, and APOBEC3G. The extended intervening loop of DddAₜₒₓ not present in APOBEC3G is shown. FIGS. 23F-23G show in vitro cytidine deamination assays using a synthetic double-stranded, as shown in FIG. 23F, or single-stranded 36-nt DNA substrate (S) containing AC, TC, CC, and GC, as shown in FIG. 23G. Cytidine deamination leads to products (P) with increased mobility (15-21nt). A3A, APOBEC3A. FIG. 23H shows the mutation frequency as measured by spontaneous rifampicin resistance emergence in the indicated E. coli strains expressing DddAₜₒₓ or catalytically inactivated DddAₜₒₓ (E1347A) *P<0.05 by unpaired two-tailed t-test. FIG. 23I shows a probability logo of the region flanking SNPs identified in five E. coli Δung isolates serially exposed to a low level of DddAₜₒₓ.

FIGS. 24A-24D show how engineering non-toxic split-DddAₜₒₓ halves can reconstitute activity when co-localized on DNA. FIG. 24A shows the rational design of seven split sites in apo-DddAtox. The zinc ion is shown in grey. DddAtox was split at the peptide bond between the labelled amino acid and the residue immediately after. FIG. 24B shows architectures of split-DddAtox halves fused to the N-terminus of orthogonal Cas9 proteins dSpCas9 and SaKKH-Cas9(D10A). DddAtox-N and DddAtox-C contain the N-terminus and C-terminus of DddAtox, respectively. Two fusion orientations (aureus-N or aureus-C) are possible for a given split. Guide RNAs are encoded on separate plasmids and transcribed from a U6 promoter. FIG. 24C shows fusions of split-DddAtox halves to orthogonal dSpCas9 and SaKKH-Cas9(D10A) enable reassembly of active DddAtox (top). If split-DddAtox halves are instead fused to the same Cas9 variant, half of reassembled DddAtox is predicted to be non-functional (bottom). FIG. 24D is a heat map of editing efficiencies for G1333 and G1397 splits at the nuclear DNA site EMX1. Each split was assayed in aureus-N and aureus-C orientations across four lengths of spacing regions. The positions of dSpCas9 (pink) and SaKKH-Cas9(D10A) (blue) protospacers are shown. At nucleotide positions containing a canonical T, indels can result in <100% T, as reflected by the heat map. Colors reflect the mean of n=2 independent biological replicates.

FIGS. 25A-25E show how to optimize mitoTALE-DddAtox array fusions for mitochondrial base editing in human cells. FIG. 25A is a schematic of unoptimized m.14459A-TALE-DddAtox array fusions that bind to DNA flanking a 15-bp spacing region in mitochondrial ND6 (see FIG. 34C for editing efficiencies). Target cytidines are shown in C11, C13, C6, and C7 and mitoTALE binding sites are shown in blue. DddAtox was split at G1397 with DddAtox-N fused to the left-side mitoTALE. The N-terminal domain of the right-side mitoTALE was engineered to recognize cytidine at the N0 position55. ND6 editing efficiencies of fusions containing 1×- or 2×-UGI proteins at the N- or C-terminus are shown below. Fusions containing bpNLS instead of MTS, or lacking any localization signal sequence, were included as negative controls. FIG. 25B shows fluorescence imaging of HA- and FLAG-tagged halves of N-terminus UGI-mitoTALE-DddAtox and C-terminus mitoTALE-DddAtox-UGI pairs in HeLa cells 24 h after plasmid transfection. Mitochondrial localization was determined by staining with Mitotracker. Scale bar, 10 RM. FIG. 25C shows optimized DdCBE architecture containing one UGI protein fused to the C-terminus of each fusion. Editing efficiencies and indel frequencies of mitochondrial-localized ND6-DdCBE targeting ND6 in mtDNA and nuclear-localized BE2 and BE4max targeting EMX1 in nuclear DNA are shown. FIG. 25D shows product purity among edited DNA sequencing reads in which the specified target C is shown for the indicated nuclear (BE2 (left) and BE4max (middle)) or mitochondrial (ND6-DdCBE) base editors. FIG. 25E is a ND6 allele frequency table obtained from HEK293T cells treated with ND6-DdCBE. All values and errors for FIGS. 25A, and 25C-25E reflect the mean±s.d. of n=3 independent biological replicates.

FIGS. 26A-26J show DdCBE editing at five mitochondrial DNA genes in human cells. Schematics of DdCBEs showing their mitoTALE repeats, target dsDNA spacing region, and split DddAtox orientation that resulted in the highest on-target editing efficiencies. Colored components are defined in FIGS. 25A-25E. Editing efficiencies of indicated DdCBE in all possible G1333 and G1397 split orientations are shown (right) for ND1-DdCBE (FIG. 26A), ND5.1-DdCBE (FIG. 26B), ND4-DdCBE (FIG. 26C), ND5.2-DdCBE (FIG. 26D), ND5.3-DdCBE (FIG. 26E), ATP8-DdCBE (FIG. 26F) and ND2-DdCBE (FIG. 26G). HEK293T cells were transfected with two plasmids, each encoding an MTS-mitoTALE array-split DddAtox-UGI half programmed to bind the mtDNA half-site shown. Genomic DNA was harvested three (FIGS. 26B, 26D, and 26F) or six days (FIGS. 26A, 26C, 26E, 26G) post-transfection and analyzed by high-throughput DNA sequencing. All values and errors in FIGS. 26A-26G reflect the mean±s.d. of n=3 independent biological replicates. FIG. 26H shows the confirmation of m.11922G>A ND4 editing by Sanger sequencing in HEK293T cells eight days after transfection with ND4-DdCBE and the catalytically inactivated ND4-DddCBE containing the E1347A mutation in split DddAtox (dead ND4-DdCBE). FIG. 26I shows the oxygen consumption rate (OCR) analyzed by XF Seahorse Analyzer. FIG. 26J shoes relative values of respiratory parameters. All values and errors in (FIG. 26I) and (FIG. 26J) reflect the mean±SEM of n=3 independent biological replicates. For FIG. 26J, asterisks indicate significant OCR difference based on a comparison between ND4-DdCBE edited and dead ND4-DdCBE mock-edited cells. *P<0.05 by Student's two-tailed unpaired t-test.

FIGS. 27A-27E show mitochondrial genome-wide off-target DNA editing profiles for DdCBEs. FIG. 27A shows HEK293T cells were transfected with plasmids encoding active DdCBE, the inactive mutant DdCBE (dead-DdCBE) containing DddAtox(E1347A) or TALE-free DddAtox halves split at G1397 (TALE-free 1397 DddAtox, with each half containing MTS-split DddAtox-UGI. 5,000-10,000 cells were harvested after 3 days for bulk-cell ATAC-seq. Each base was sequenced with an average of 5,100-9,900× coverage. The inner circle in the radial plot represents 5,000× coverage and the numbers represent the positions of the human mitochondrial genome. FIG. 27B shows the average % frequency of genome-wide C·G-to-T·A off-target editing in mtDNA by indicated DdCBE and controls (see Methods for quantifying average off-target editing frequencies). The dashed line represents the frequency of endogenous C·G-to-T·A conversions in mtDNA as measured in the untreated control. FIG. 27C shows the number of high-confidence off-target SNVs identified after treatment with the indicated DdCBE or in control samples (see Methods for variant calling workflow, and FIGS. 39A-39F for on- and off-target editing efficiencies of individuals SNVs for each DdCBE). FIG. 27D shows sequence logos generated from off-target C·G-to-T·A conversions by each indicated DdCBE and TALE-free G1397 DddAtox. The target cytidine is at position 21. The 20 bases upstream and downstream of the deaminated cytidine represent TALE array binding sites flanking the spacing region that contains the target base. Bits reflect sequence conservation at a given position. FIG. 27E is a Venn diagram⁷⁷ depicting the number of off-target SNVs shared among two or more DdCBEs. The combined number of unique SNVs for each DdCBE from all three independent biological replicates is indicated in parenthesis. All values and errors in (FIG. 27B) and (FIG. 27C) reflect the mean±SEM of n=3 independent biological replicates.

FIGS. 28A-28C show that DddA is encoded adjacent to a predicted immunity gene and exhibits bactericidal activity during interbacterial competition. FIG. 28A shows genomic context of dddA and dddIA in B. cenocepacia H111. FIG. 28B shows the viability of B. cenocepacia ΔdddA ΔdddIA (recipient) over time during competition with B. cenocepacia donor strains carrying wild-type dddAtox or dddAtoxE1347A. Data represent the mean±s.d. of n=2 technical replicates indicative of at least three biological replicates. FIG. 28C shows a α-VSV-g western blot analysis of total cell lysates of E. coli expressing the indicated deaminases tagged with VSV-G epitope. RNAP-β is used as a loading control.

FIGS. 29A-29C show an analysis of DddAtox activity against dsDNA and RNA substrates. FIG. 29A shows an in vitro DNA cytidine deamination assays using double-stranded 36-nt DNA substrates containing AC, TC, CC, and GC with a FAM fluorophore on the forward (A) or reverse (B) strand. Deamination activity results in a cleavage product (P). FIG. 29B and FIG. 29C show a poisoned primer extension assay to detect deamination of cytidine in single-(FIG. 29B) or double-(FIG. 29C) stranded RNA substrates. A mix of RNA substrates containing the sequences GUCG or GUUG at the indicated ratios were incubated with purified DddAtox and reverse transcriptase. Primer extension was performed in reactions with ddGTP to terminate primer extension at cytidine residues. Cytidine deamination yields the 31-mer product.

FIGS. 30A-30B show predicted nucleotide interactions of DddAtox compared to those of APOBEC3A. FIG. 30A is a schematic showing an electrostatic surface potential rendering of human APOBEC3A in complex with single-strand DNA (PDB 5SWW)⁷⁸. FIG. 30B is a model for DddAtox (electrostatic surface rendering) interaction with double-stranded DNA, based on superposition with the APOBEC3A structure. The substrate cytidine is shown for (FIG. 30A) and (FIG. 30B).

FIGS. 31A-31D show that DddAₜₒₓ deaminate cytidines in bacteria and exhibit sequence context preference. FIG. 31A shows the number of SNPs from the indicated nucleotide classifications observed in E. coli Δudg following intoxication with DddAtox or DddAtox(E1347A). FIGS. 31B-31C show the position of SNPs on the chromosome of E. coli Δudg isolates intoxicated with DddAtox (FIG. 31B) or DddAtox(E1347A) (FIG. 31C). FIG. 31D shows a deamination assay on DddAtox with double-stranded DNA substrates containing a single C with different nucleotides (A, T, C, or G) at the position immediately 5′ of the C (fourth nucleotide as read left to right) (S, substrate; P, product).

FIGS. 32A-32H show base editing efficiencies of all seven DddAₜₒₓ splits. Each split was assayed in the aureus-N and aureus-C orientation (see FIG. 24B) across spacing region lengths of 12-bp (FIG. 32A), 17-bp (FIG. 32B), 23-bp (FIG. 32C), 28-bp (FIG. 32D), 33-bp (FIG. 32E), 39-bp (FIG. 32F), 44-bp (FIG. 32G) and 60-bp (FIG. 32H). Cytidines that are deaminated by reassembled DddAtox are represented on the heat map. At nucleotide positions containing a canonical T, indels can result in <100% T, as reflected on the heat map. Colors reflect the mean of n=2 independent biological replicates.

FIGS. 33A-33D show how TALE-split-DddAₜₒₓ proteins mediate efficient base editing in nuclear DNA of human cells. FIG. 33A is a schematic of TALE-split DddAtox fusion variants that bind to DNA flanking the 18-bp spacing region at the nuclear CCR5 site in U20S cells. The target C is shown in C9, C10, and C16 and TALE binding sites are shown as nucleotides 1-11 of the top strand as read 5′ to 3′, and nucleotides 1-12 of the bottom strand as read 5′ to 3′. Editing efficiencies and indel frequencies for TALE-split-DddAtox pairs in G1333 and G1397 split orientations are shown. FIG. 33B shows the architecture of CCR5-DdCBE. This architecture was optimized for DdCBEs targeting mtDNA. Target cytidines within the CCR5 spacing region are shown. FIG. 33C shows the editing efficiencies and indel frequencies of U20S cells treated with CCR5-DdCBE and ND6-DdCBE are shown. Dead-DdCBEs containing the inactivating DddAtox(E1347A) mutation were used as negative controls. Cells were harvested 3 days-post transfection for DNA sequencing. FIG. 33D shows the outcomes among edited alleles in which the specified target C is mutated are shown for indicated base editor. Values and error bars in FIGS. 33A, 33C, and 33D reflect the mean±s.d. of n=3 independent biological replicates.

FIGS. 34A-34C show unoptimized mitoTALE-split DddAtox fusions mediate modest editing of mitochondrial ND6 in HEK293T cells. FIG. 34A shows the architectures of non-UGI containing ND6-mitoTALE-DddAtox fusion pair. DddAtox was split at G1333 or G1397. TALEs bind to mtDNA sequences (blue) that flank a 15-bp spacing region in mitochondrial ND6. Mutations in the N-terminal domain (NTD)⁷¹ of the Right-TALE should permit recognition of C in addition to canonical T at the first nucleotide bound by the TALE array (the N0 position). Target cytidines are shown in purple. The last TALE repeat marked with an asterisk did not match the reference genome (see Supplementary Table 4). FIG. 34B shows mtDNA editing efficiencies of mitoTALE-DddAtox pairs in the listed split orientations. The dashed line is drawn at 0.1%. FIG. 34C zmLOC100282174, a Zea mays-derived MTS⁷⁵, was appended before or after SOD2 and COX8A MTS sequence of each MTS-mitoTALE-split DddAtox-UGI fusion. All editing efficiencies are measured 3 days post-transfection. Values and error bars in FIGS. 34B and 34C reflect the mean±s.d. of n=3 independent biological replicates

FIGS. 35A-35C show how DdCBE editing in the nucleus of U20S cells yields more indels and lower product purity compared to editing in the mitochondria. FIG. 35A Architecture of CCR5-DdCBE. DddAtox was split at G1333 with DddAtox-N fused to the left-side CCR5-targeting TALE (see FIG. 33A for editing efficiencies of other split orientations in the absence of UGI protein). The bpNLS sequence directs the localization of the CCR5-targeting DdCBE to the nucleus. Target cytidines within the CCR5 spacing region are shown in C9, C10, ad C16. FIG. 35B shows editing efficiencies and indel frequencies of U20S cells treated with CCR5-DdCBE and ND6-DdCBE are shown. The inactive mutant DdCBEs (dead-DdCBE) containing DddAtox(E1347A) were used as negative controls. FIG. 35C shows product purity among edited DNA sequencing reads in which the specified target C is mutated is shown for nuclear CCR5-DdCBE and mitochondrial ND6-DdCBE. All values and errors in (FIG. 35B) and (FIG. 35C) reflect the mean±s.d. of n=3 independent biological replicates.

FIGS. 36A-36C show the effect of DdCBE editing on cell viability and mitochondrial DNA integrity. FIG. 36A shows cell viability was measured by luminescence at indicated timepoints using the CellTiter-Glo 2.0 assay (Promega). Luminescence values were normalized to untreated control. FIG. 36B shows that purified genomic DNA was isolated from DdCBE-treated HEK293T cells at indicated timepoints and amplified by PCR with mtDNA-specific primers to capture the entire 16.6 kb mtDNA as two amplicons (shown between the arrows as bounded by the tails). DNA gel images are representative of n=3 independent biological replicates (see FIG. 51 for uncropped images). FIG. 36C shows relative mtDNA levels in cells treated with indicated DdCBE were measured by quantitative PCR at various timepoints (see the Supplementary Sequences section of Example 1 for PCR primers). All values and errors in (FIG. 36A) and (FIG. 36C) reflect the mean±s.d of n=3 independent biological replicates.

FIGS. 37A-37F show that targeted DdCBE editing in mtDNA of HEK293T cells persist over multiple cell divisions. Editing efficiencies for ND6-DdCBE (FIG. 37A), ND5.1-DdCBE (FIG. 37B), ND5.2-DdCBE (FIG. 37C), ATP-DdCBE (FIG. 37D), BE2 and BE4max (FIG. 37E) in HEK293T cells are shown for each timepoint. For each DdCBE (FIGS. 37A-37D), the optimized split orientation is listed in parenthesis. C·G-to-T·A conversions at protein-coding genes that generate missense mutations (first amino acid as read left to right, shown at the tail-end of each arrow) of the putative amino acid (second amino acid as read left to right, shown at the head of each arrow) are shown. All values and errors reflect the mean±s.d. of n=3 independent biological replicates. For (FIGS. 37A-37E), asterisks indicate significant editing based on a comparison between indicated time points. *P<0.05 and **P<0.01 by Student's two-tailed paired t-test. Individual P values are listed in Table 3. FIG. 37F shows a western blot of ND6-, ND5.1-, ND5.2-, and ATP8-DdCBE at various timepoints from crude HEK293T cell lysates. The right halves were FLAG-tagged and the left halves were HA-tagged. DdCBE halves are distinguished by their molecular weight (see FIG. 52 for uncropped images and fluorescent tagging of each half). Nuclear 3-actin was used as loading control.

FIGS. 38A-38K show the effects of ND4-DdCBE editing on mtDNA homeostasis. FIG. 38A shows mtDNA levels of ND4-edited cells measured by quantitative PCR (qPCR) relative to mock-edited cells. FIG. 38B shows mtRNA levels of ND4-edited cells measured by RT-qPCR relative to mock-edited cells. All values and errors reflect the mean±SEM of n=3 independent biological replicates. Student's unpaired two-tailed t-test was applied. ns, not significant (P>0.05). FIG. 38C shows the confirmation of m.13494C>T ND5 editing by Sanger sequencing and Illumina DNA sequencing in cells transfected with ND5.1-DdCBE. Non-transfected cells were used as a control. FIG. 38B shows the oxygen consumption rate (OCR) of cells treated with ND5.1-DdCBE. ND5.1-DdCBE; (left-hand column of each pair of columns) non-transfected control. FIG. 38C shows the relative values of respiratory parameters of ND5.1-DdCBE-treated cells. Sanger sequencing, Illumina DNA sequencing and OCR of cells treated with ND6-DdCBE (FIG. 38D), ND5.2-DdCBE (FIG. 38E), ND5.3-DdCBE (FIG. 38F), ND2-DdCBE (FIG. 38G), ND1-DdCBE (FIG. 38H) and ATP8-DdCBE (FIG. 381) are shown. All cells were harvested 6 days post-transfection. All values and error bars shown in the graphs reflect the mean±SEM of n=3 independent biological. For (FIG. 38B) and (FIG. 38E), Student's unpaired two-tailed t-test was applied. ns, not significant (P>0.05).

FIGS. 39A-39F show average frequencies of each on-target (colored) and off-target (grey) SNV and their positions within the NC_012920 reference human mtDNA are shown for 5,000-10,000 cells treated with ND6-DdCBE (FIG. 39A), ND5.1-DdCBE (FIG. 39B), ND5.2-DdCBE (FIG. 39C), ND4-DdCBE (FIG. 39D), or ATP8-DdCBE (FIG. 39E). FIG. 39F shows SNP alleles and their associated average frequencies are listed for the dead-DdCBEs, TALE-free G1397 DddAtox, and untreated controls. For (FIGS. 39A-39E), dead DdCBEs, and TALE-free G1397 DddAtox, the combined number of unique off-target SNVs from all three independent biological replicates that are absent in the untreated control are shown. For the untreated control, heteroplasmic mutations were excluded. Average frequencies were calculated from three independent biological replicates.

FIGS. 40A-40C show that intact DddAₜₒₓ fused to DNA-binding protein is toxic to human cells. FIG. 40A shows architectures of BE2, BE4max and intact DddAtox-Cas9 fusions. The DddAtox-Cas9 linker lengths tested were 32-, 10- and 5-amino acids residues. Rigid linkers contain amino acids EAAAK (SEQ ID NO: 108) or EAAAKEAAAK (SEQ ID NO: 108). The flexible linker contains amino acids GGGGSGGGGS (SEQ ID NO: 344). Proteins expression was induced by the addition of 0.1 μg/mL doxycycline 2-4 h after plasmid transfection. FIG. 40B shows cell viability was measured by luminescence every 24 h after transfection for 3 days using the CellTiter-Glo 2.0 assay (Promega). Luminescence values were normalized to untreated control. Values reflect the mean±s.e.m. of n=2 independent biological replicates, each performed in technical triplicates. FIG. 40C shows Cas9 binds to EMX1 protospacer (underlined) upstream of the PAM (nucleotides 41-43 of the top strand as read 5′ to 3′, which is also the first three nucleotides following the underlined segment as read 5′ to 3′ consisting of “TGG”) and unwinds DNA to expose single-stranded DNA containing cytidines (nucleotides 21-23 and 27-29 of the top strand as read 5′ to 3′) that are substrates for C-to-T editing by BE2 and BE4max. Editing efficiencies for BE2 and BE4max 3 days post-transfection are shown below. The dsDNA regions flanking the EMX1 protospacer contain 5′-TC-3′ bases (nucleotides 2, 4, 7, 17, and 46 of the top strand as read 5′ to 3′, and 3, 6, and 12 of the bottom strand as read 5′ to 3′) which can act as deamination substrates for DddAtox-Cas9 fusions. Values and errors reflect the mean±s.d. of n=2 independent biological replicates.

FIGS. 41A-41C show the design of guide RNAs for split-DddAₜₒₓ-Cas9 screen. FIG. 41A is a schematic of relative binding sites for dSpCas9 and SaKKH-Cas9(D10A) gRNAs targeting the EMX1 loci. The gRNAs position the orthogonal split-DddAtox-Cas9 fusions adjacent to each other for DddAtox reconstitution and deamination of a target TC base within the dsDNA spacing region. FIG. 41B is a table showing the pairing of dSpCas9 guide RNAs (spG7 and spG6) with SaKKH guide RNAs (saG1 to saG4) to generate spacing regions with lengths between 12 and 60 bp. FIG. 41C shows the sequences of guide RNAs listed in (FIG. 41B). PAM sequences of dSpCas9 guide RNAs are shown as initial (most right left-side) 3 nucleotides of SEQ ID NO: 550-551 and PAM sequences of SaKKH guide RNAs are shown as the terminal (most right hand-side) 6 nucleotides of SEQ ID NO: 552-555.

FIGS. 42A-42B show how editing strictly depends on reassembly of split-DddAtox-Cas9 halves at target site. FIG. 42A shows base percentages at each position of the EMX1 locus are shown for all tested split orientations with no guide RNAs for dSpCas9 and SaKKH-Cas9(D10A). The nucleotide percentages for G1397 aureus-N split with gRNAs flanking a 23-bp target spacing is shown as a reference. Arrow indicates the position of C-to-T editing within the spacing region (see FIG. 24D and FIGS. 32A-32H for expected editing efficiencies of all split orientations in the presence of guide RNAs). FIG. 42B shows that, for G1333 and G1397 splits, DddAtox-dSpCas9 or DddAtox-SaKKH-Cas9(D10A) halves were directed to a site within EMX1 by a guide RNA spG4 or saG4, respectively. The reciprocal DddAtox half of each fusion was absent. Target TC bases were present 12-21 bp upstream of the protospacer. Shown are the base percentages at each position of the EMX1 locus. All nucleotide percentage plots are representative of n=2 independent replicates.

FIG. 43 shows indel frequencies of split-DddAₜₒₓ-Cas9 fusions. Percent of indels for the seven DddAₜₒₓ splits across the indicated length of spacing region. Percent of indels among total sequencing reads for the seven DddAtox splits across the indicated length of spacing region. Each split was tested in the aureus-C and aureus-N orientations (see FIG. 24B for architectures). All values reflect the mean±s.d. of n=2 independent biological replicates.

FIG. 44 shows dual MTS sequences do not improve mtDNA editing efficiencies. zmLOC100282174, a Zea mays-derived MTS10, was appended before or after SOD2 and COX8A MTS sequence of each MTS-mitoTALE-split DddAtox-UGI fusion. Mitochondrial editing percentages at ND6 are shown for the indicated MTS sequence combination. Values and errors reflect the mean±s.d. of n=3 independent biological replicates.

FIGS. 45A-45B show indel frequencies of DdCBEs in their optimized orientations. Shown are the percent of indels for each optimized DdCBE that produced the highest editing efficiency when DddAtox was split at G1397 (FIG. 45A) or G1333 (FIG. 45B) (see FIGS. 26A-26J for on-target editing efficiencies of optimized DdCBEs). All values and errors reflect the mean±s.d. of n=3 independent biological replicates.

FIGS. 46A-46E show that targeted editing in the mitochondrial DNA of U20S cells persists over multiple cell divisions. Editing efficiencies for ND6-DdCBE (FIG. 46A), ND5.1-DdCBE (FIG. 46B), ND5.2-DdCBE (FIG. 46C), ATP-DdCBE (FIG. 46D) and CCR5-DdCBE (FIG. 46E) in U20S cells are shown for each timepoint. For each DdCBE, the optimized split orientation is provided in parenthesis. C·G-to-T·A conversions at protein-coding genes that generate missense mutations (green) of the putative amino acid (red) are shown. All values and errors reflect the mean±s.d. of n=3 independent biological replicates. Asterisks indicate significant editing based on a comparison between indicated time points. *P<0.05, **P<0.01 and ***P<0.001 by Student's two-tailed paired t-test. Individual P values are listed in Table 3.

FIG. 47 shows sequencing coverage of ATAC-seq samples. Per-base sequencing coverages of each replicate treated with DdCBEs, dead-DdCBE, TALE-Free G1397 DddAtox and untreated control. The nucleotide positions of the human mitochondrial DNA from the NC_012920 reference genome are indicated in the exterior of each radial plot. Inner circle represent 5,000× coverage.

FIGS. 48A-48B show expression levels of different DdCBEs over three days. FIG. 48A shows western blots of ND6-, ND5.1-, ND5.2-, and ATP8-DdCBE at one-day intervals from crude HEK293T cell lysates over a three-day time course. The right-hand half was FLAG-tagged and the left-hand half was HA-tagged. DdCBE halves are distinguished by their molecular weight (see FIG. 53 for uncropped images and fluorescent tagging of each half). Nuclear 3-actin was used as loading control. Images are representative of n=3 independent biological replicates. FIG. 48B shows proteins levels were normalized to nuclear 3-actin and quantified by densitometry using ImageJ. Asterisks indicate significant editing based on a comparison between indicated time points. *P<0.05 by Student's two-tailed unpaired t-test. Values and errors reflect the mean±s.d. of n=3 independent biological replicates.

FIGS. 49A-49C show the predicted effects of off-target SNVs on mitochondrial DNA sequence and protein function. FIG. 49A shows a classification of off-target SNVs into noncoding or coding mutations. Mutations occurring in protein-coding regions of mtDNA were further categorized into synonymous, missense or nonsense mutations. FIG. 49B shows, for nonsynonymous SNVs, SIFT was used to predict the effect of these mutations on protein function. High- or low-confidence calls (indicated in parentheses) were made according to the standard parameters of the prediction software. FIG. 49C shows editing frequencies of selected off-target TC bases in the indicated sequence contexts following targeted amplicon sequencing. Values and errors reflect the mean±s.d. of n=3 independent biological replicates.

FIG. 50 shows the percentage of base pair changes needed to reverse pathogenic mtDNA point mutations in the MITOMAP database¹² (accessed Dec. 10, 2019). Disease-associated mutations in rRNA/tRNA and coding/non-coding regions were considered only if they had been assigned ‘Cfrm’ statuses. (see Table 6 for list of 83 pathogenic mtDNA SNPs).

FIG. 51 shows the uncropped images for FIG. 36B.

FIG. 52 shows dual fluorescence imaging of SOD2 MTS¬-left TALE-split DddAtox-UGI half and COX8A MTS¬-right TALE-split DddAtox-UGI half for each DdCBE (see FIG. 37F). The uncropped images for FIG. 37F are shown on the right.

FIG. 53 shows the dual fluorescence imaging of SOD2 MTS-left TALE-split DddAₜₒₓ-UGI half and COX8A MTS-right TALE-split DddAₜₒₓ-UGI half for each DdCBE (see FIGS. 48A-48B). For expression of TALE-free split-DddAtox, G1397-DddAₜₒₓ-N and G1333-DddAtox-N appear as bands; G1397-DddAₜₒₓ-C and G1333-DddAₜₒₓ-C appear as bands. The uncropped images for FIGS. 48A-48B are shown on the right.

FIGS. 54A-54C show that stalling mtDNA replication impairs mitochondrial base editing in human cells. FIG. 54A is a schematic of experimental design. Addition of doxycycline (Dox) induces the stable expression of a dominant-negative mutant of DNA polymerase-gamma containing a D1153A substitution (POLGdn) in a HEK293-derived cell line⁵⁷. Total cell lysate was collected at indicated timepoints for western blotting of POLGdn in triplicates. FIG. 54B shows mtDNA levels of uninduced (no Dox) and induced (+Dox) cells treated with indicated DdCBE 2 days post-transfection. mtDNA levels were measured by quantitative PCR (qPCR) and normalized to uninduced cells without DdCBE treatment. FIG. 54C shows the editing efficiencies of indicated DdCBE in uninduced and induced cells 48 hours post-transfection. All values and error bars in (FIG. 54B) and (FIG. 54C) reflect the mean±s.d of n=3 independent biological replicates.

FIGS. 55A-55C show the off-target editing activity of DdCBEs in nuclear DNA of human HEK293T cells. The on-target editing site in mtDNA and the corresponding nuclear DNA sequence with the greatest homology are shown for ND6-DdCBE (FIG. 55A), ND5.1-DdCBE (FIG. 55B), and ND4-DdCBE (FIG. 55C). TALE binding sites begin at NO and are shown. Target cytidines are in C7, C8, C11, and C13. Nucleotide mismatches between the mtDNA and nuclear pseudogene are shown. Editing efficiencies are measured by targeted amplicon sequencing 3 days post-transfection (FIGS. 55A-55B) or six days post-transfection (FIG. 55C) (see Methods for primer sequences). Each amplicon was sequenced at >44,000× coverage. All values and error bars reflect the mean±s.d of n=3 independent biological replicates. Student's unpaired two-tailed t-test was applied. ns, not significant (P>0.05).

FIGS. 56A-56B show TALE arrays need to bind to mtDNA sequences positioned in close proximity to reassemble catalytically active DddAₜₒₓ for off-target editing. FIG. 56A shows the identities and relative binding positions of each mismatched (MM) TALE-DddAₜₒₓ half is shown. MM-1 and MM-2 contain a TALE-bound DddAₜₒₓ half and a TALE-free DddAₜₒₓ half. MM-3 and MM-4 contain DddAₜₒₓ halves fused to TALE repeat arrays that bind to distant mtDNA sites. Note that m.14459-Right TALE contains a permissive N-terminal domain. FIG. 56B shows the average percentage of genome-wide C·G-to-T·A off-target editing in mtDNA by indicated DdCBE and MM pairs. The dashed line represents the percentage of endogenous C·G-to-T·A conversions in mtDNA as measured in the untreated control. Values and error bars reflect the mean±SEM of n=3 independent biological replicates.

FIGS. 57A-57C show the predicted effects of off-target SNVs on mitochondrial DNA sequence and protein function. FIG. 57A shows the classification of off-target SNVs into noncoding or coding mutations. Mutations occurring in protein-coding regions of mtDNA were further categorized into synonymous, missense or nonsense mutations. FIG. 57B shows that for nonsynonymous SNVs, SIFT was used to predict the effect of these mutations on protein function. High- or low-confidence calls were made according to the standard parameters of the prediction software. FIG. 57C shows the editing efficiencies of selected off-target TC bases in the indicated sequence contexts. HEK293T cells were treated with indicated DdCBE and harvested 3 days post-transfection for targeted amplicon sequencing. Values and error bars reflect the mean±s.d. of n=3 independent biological replicates.

FIG. 58 is a schematic of relative binding sites for dSpCas9 and SaKKH-Cas9(D10A) gRNAs targeting the EMX1 loci. The gRNAs position the orthogonal split-DddAtox-Cas9 fusions adjacent to each other for DddAtox reconstitution and deamination of a target TC base within the dsDNA spacing region.

FIG. 59 is a schematic showing the selection circuit in PANCE or PACE for evolving split DddA towards higher activity at TC context. DdCBE is encoded in M13 bacteriophage. Plasmid P3 is in the E. coli host cell and encodes for T7 RNA polymerase (T7 RNAP) fused to a degron. TALE-3 and TALE-4 target DNA sequences flanking a linker region within the T7 RNAP-degron fusion. Successful base editing at the linker sequence introduces a stop to remove the degron from T7 RNAP during translation. T7 RNAP is restored and binds to the T7 promoter on Plasmid P4 to drive gIII. Since gIII is required for phage infectivity, phages containing active DdCBEs will propagate and overtime.

FIGS. 60A-60D show editing activity of DdCBE mutants in mammalian HEK293T cells. FIG. 60A shows DdCBE protein architecture used to test mutant activity. FIGS. 60B-60C show editing efficiencies of DdCBEs targeting MT-ATP8, MT-ND5.2 and MT-ND4 3-days post transfection. FIG. 60D shows indel percentages associated with DdCBE editing.

FIG. 61 is a schematic showing DdCBE is packaged into two lentiviral vectors for transduction into mouse embryonic fibroblasts.

FIG. 62 shows Sanger sequencing of mtDNA from mouse embryonic fibroblasts treated with indicated DdCBEs. Arrow indicates the target GC base pair. The appearance of a light trace at the target G position indicates C·G-to-T·A conversion. Off-target bystander mutations are indicated in asterisks. Outlined in bold indicate the DdCBE orientation that resulted in the highest C·G-to-T·A conversion.

FIG. 63 shows results of mitochondria function characterization of edited MEF cells. For each mutation installed, the oxygen consumption rate (OCR) measurements and extracellular acidification rate (ECAR) are shown in the top and bottom panels, respectively.

FIGS. 64A-64B are schematics comparing the different DdCBE architectures for target mtDNA binding. FIG. 64A shows that Left-TALE binds to top strand and Right-TALE binds to bottom strand. Each UGI protein is in close proximity to the target spacing region. FIG. 64B shows schematics of “opposite”, “top” and “bottom” architecture. “Opposite” shows Left-TALE binds to bottom strand and Right-TALE binds to top strand. Both UGI proteins are distal to the target spacing region. “Top” shows both TALE proteins bind to the top strand. “Bottom” shows both TALE proteins bind to the bottom strand. In top and bottom architecture, only one UGI protein is close to the target site.

FIG. 65 shows editing activity of alternative DdCBE architectures in mammalian HEK293T cells. Heatmaps showing C·G-to-T·A conversion at MT-ND1 for Original, Top, Bottom and Opposite architectures. Each architecture was tested in its four possible DddA split orientations. “*” indicates that the cytidine C is within the TALE binding site.

FIG. 66 shows binding motifs used in ZF-BE design (Gersbach et al. Acc. Chem. Res. 2014. 7(8);23309-2318).

FIG. 67 shows the sequence targeted at site R8 in the human mitochondrial genome by ZFs R8, 5×ZnF-4-R8 and 5×ZnF-18-R8.

FIGS. 68A-68D show editing activity for various ZFs designed to create a 4-18 bp editing window with ZF-R8.

FIG. 69 shows the sequence targeted at site R13 in the human mitochondrial genome by ZFs R13, 5×ZnF-4-R13 and 5×ZnF-18-R13.

FIGS. 70A-70D show editing activity for various ZFs designed to create a 4-18 bp editing window with ZF-R13.

FIGS. 71A-71B show improvements to ZF-BE architecture made in round 2 of optimization.

FIG. 72 shows the sequence targeted at site R13 in the human mitochondrial genome by ZFs R13, 5×ZnF-9-R13 and 5×ZnF-12-R13.

FIGS. 73A-73B show editing activity in human HEK293T cells targeting site R13. Results show the differences in outcomes from using ZF-BE architectures as described in FIG. 71A.

FIG. 74 shows the sequence targeted at site R8 in the human mitochondrial genome by ZFs R8, 5×ZnF-4-R8 and 5×ZnF-10-R8.

FIGS. 75A-75B editing activity in human HEK293T cells targeting site R8. Results show the differences in outcomes from using ZF-BE architectures as described in FIG. 71A.

FIG. 76 shows improvements in ZF-BE architecture from round 3 of optimization.

FIGS. 77A-77D show editing activity in human HEK293T cells targeting sites R8 and R13. Results show the differences in outcomes from using ZF-BE architectures as described in FIG. 76.

FIG. 78 shows improvements in ZF-BE architecture from round 4 of optimization.

FIGS. 79A-79D show editing activity in human HEK293T cells targeting sites R8 and R13. Results show the differences in outcomes from using ZF-BE architectures as described in FIG. 78.

FIGS. 80A-80D show editing activity in human HEK293T cells targeting sites R8 and R13. Results show the differences in outcomes from using modified UGI homologs in ZF-BE architectures as described in FIG. 76.

FIGS. 81A-81C show exemplary ZF scaffolds with non-conserved positively charged residues in bold. FIG. 81C shows mutations used in round 4 of optimization.

FIGS. 82A-82D show editing activity in human HEK293T cells targeting sites R8 and R13. Results show the differences in outcomes in mutated ZF scaffolds from using ZF-BE architectures as described in FIG. 78.

FIG. 83 shows the improvements in ZF-BE architecture from round 5 of optimization.

FIGS. 84A-84D show editing activity in human HEK293T cells targeting sites R8 and R13. Results show the differences in outcomes from using ZF-BE architectures as described in FIG. 83.

FIGS. 85A-85D show editing activity in human HEK293T cells targeting sites R8 and R13. Results show the differences in outcomes from using ZF-BE architectures as described in FIG. 83.

FIG. 86 shows the improvements in ZF-BE architecture from rounds of optimization. v6 differs from v3 in the inclusion of an additional NES, improvement of the ZF scaffold sequence, and coexpression of a separate mitochondrially-targeted UGI. v6M differs from v6 in the inclusion of mutations T1380I, E1396K and T1413I into the split DddA deaminase halves.

FIGS. 87A-87B show the sequence targeted at site R13 in the human mitochondrial genome by ZFs R13-1, 5×ZnF-9-R13 and 5×ZnF-12-R13-1 (FIG. 87A), and editing activity of ZF-BEs in human HEK293T cells targeting sites R8 and R13 three days post-transfection (FIG. 87B). Results show the differences in outcomes from using ZF-BE architectures as described in FIG. 86.

FIG. 88 is an exemplary schematic showing a TALE-DdCBE target for alternative UGI homologs.

FIG. 89 is a schematic showing the experimental design for testing alternative UGI homologs.

FIGS. 90A-90D show editing activity of TALE-DdCBEs in human HEK293T cells targeting sites ND4, ND5.1 and ATP8 three days post-transfection. Results show the differences in outcomes from using different UGI homologs in comparison against the canonical UGI sequence from bacteriophage PBS2 (UGIcontrol).

FIGS. 91A-91D show results of alternative UGI homolog testing in BE4max.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

### AAV

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

rAAV particles may comprise a nucleic acid vector (e.g., a recombinant genome), which may comprise at a minimum: (a) one or more heterologous nucleic acid regions comprising a sequence encoding a protein or polypeptide of interest (e.g., a split Cas9 or split nucleobase) or an RNA of interest (e.g., a gRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector further comprises a region encoding a Rep protein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

### Adenosine Deaminase

As used herein, the term “adenosine deaminase” or “adenosine deaminase domain” refers to a protein or enzyme that catalyzes a deamination reaction of an adenosine (or adenine). The terms are used interchangeably. In certain embodiments, the disclosure provides base editor fusion proteins comprising one or more adenosine deaminase domains. For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be enzymes that convert adenine (A) to inosine (I) in DNA or RNA. Such adenosine deaminase can lead to an A:T to G:C base pair conversion. In some embodiments, the deaminase is a variant of a naturally occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5,6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5,6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

### Antisense Strand

In genetics, the “antisense” strand of a segment within double-stranded DNA is the template strand, and which is considered to run in the 3′ to 5′ orientation. By contrast, the “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Base Editing

“Base editing” refers to genome editing technology that involves the conversion of a specific nucleic acid base into another at a targeted genomic locus (e.g., including in a mtDNA). In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

### Base Editor

The term “base editor (BE)” as used herein, refers to an agent comprising a polypeptide that is capable of making a modification to a base (e.g., A, T, C, G, or U) within a nucleic acid sequence (e.g., mtDNA) that converts one base to another (e.g., A to G, A to C, A to T, C to T, C to G, C to A, G to A, G to C, G to T, T to A, T to C, T to G). In some embodiments, the BE refers to those fusion proteins described herein which are capable of modifying bases directly in mtDNA. Such BEs can also be referred to herein as “mtDNA base editors” or “mtDNA BEs.”0 Such BEs can refer to those fusion proteins comprising a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in mtDNA, rather than destroying the mtDNA with double-strand breaks (DSBs). It should be noted that in some places DddA is referred to as DddE (e.g., FIG. 6 of the accompanying drawings). In these instances, DddE shall be interpreted to refer to DddA as a synonym.

In some embodiments, the base editors contemplated herein comprise a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017 and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28;152(5):1173-83 (2013)).

BEs that convert a C to T, in some embodiments, comprise a cytidine deaminase (e.g., a double-stranded DNA deaminase or DddA). A “cytidine deaminase” (including those DddAs disclosed herein) refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T nucleobase editor comprises a dCas9 or nCas9 fused to a cytidine deaminase. In some embodiments, the cytidine deaminase domain is fused to the N-terminus of the dCas9 or nCas9.

In some embodiments, the nucleobase editor further comprises a domain that inhibits uracil glycosylase, and/or a nuclear localization signal.

Cas9 domains used in base editing have been described in the following references, the contents of which may be applied in the instant disclosure to modify and/or include in BEs described herein, which can target mtDNA, e.g., in Rees & Liu, Nat Rev Genet. 2018; 19(12):770-788 and Koblan et al., Nat Biotechnol. 2018; 36(9):843-846; as well as. U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Publication No. WO 2018/0176009, published Sep. 27, 2018, International Application No PCT/US2019/033848, filed May 23, 2019, International Application No. PCT/US2019/47996, filed Aug. 23, 2019; International Application No. PCT/US2019/049793, filed Sep. 5, 2019; U.S. Provisional Application No. 62/835,490, filed Apr. 17, 2019; International Application No. PCT/US2019/61685, filed Nov. 15, 2019; International Application No. PCT/US2019/57956, filed Oct. 24, 2019; U.S. Provisional Application No. 62/858,958, filed Jun. 7, 2019; International Publication No. PCT/US2019/58678, filed Oct. 29, 2019, the contents of each of which are incorporated herein by reference in their entireties.

Exemplary adenine and cytosine base editors are also described in Rees & Liu, Base editing: precision chemistry on the genome and transcriptome of living cells, Nat. Rev. Genet. 2018; 19(12):770-788; as well as U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, PCT Application PCT/US2017/045381, filed Aug. 3, 2017, which published as WO 2018/027078, and PCT Application No. PCT/US2019/033848, which published as WO 2019/226953, each of which is herein incorporated by reference. Any of the deaminase components of these adenine or cytidine BEs could be modified using a method of directed evolution (e.g., PACE or PANCE) to obtain a deaminase which may use double-stranded DNA as a substrate, and thus, which could be used in the BEs described herein which are intended for use in conducting base editing directly on mtDNA, i.e., on a double-stranded DNA target.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28;152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28;152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 28). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 28). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 28). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 28).

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

cDNA

The term “cDNA” refers to a strand of DNA copied from an RNA template. cDNA is complementary to the RNA template.

### Circular Permutant

As used herein, the term “circular permutant” refers to a protein or polypeptide comprising a circular permutation, which is change in the protein's structural configuration involving a change in order of amino acids appearing in the protein's amino acid sequence. In other words, circular permutants are proteins that have altered N- and C-termini as compared to a wild-type counterpart, e.g., the wild-type C-terminal half of a protein becomes the new N-terminal half. Circular permutation (or CP) is essentially the topological rearrangement of a protein's primary sequence, connecting its N- and C-terminus, often with a peptide linker, while concurrently splitting its sequence at a different position to create new, adjacent N- and C-termini. The result is a protein structure with different connectivity, but which often can have the same overall similar three-dimensional (3D) shape, and possibly include improved or altered characteristics, including, reduced proteolytic susceptibility, improved catalytic activity, altered substrate or ligand binding, and/or improved thermostability. Circular permutant proteins can occur in nature (e.g., concanavalin A and lectin). In addition, circular permutation can occur as a result of posttranslational modifications or may be engineered using recombinant techniques. Any of the polypeptides contemplated for use in the mtDNA base editors disclosed herein may be converted to circular permutant variants, including any pDNAbp (e.g., Cas9, mitoTALE, or mitoZFP) and any double-stranded DNA deaminase (e.g., DddA).

Circularly Permuted napDNAbp

In the case of circular permutant Cas9s or other napDNAbps that could be used with the mtDNA base editors contemplated herein, the term “circularly permuted napDNAbp” refers to any napDNAbp protein, or variant thereof (e.g., SpCas9), that occurs as or engineered as a circular permutant, whereby its N- and C-termini have been topically rearranged. Such circularly permuted proteins (“CP-napDNAbp”, such as “CP-Cas9” in the case of Cas9), or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The instant disclosure contemplates any previously known CP-Cas9 or use a new CP-Cas9 so long as the resulting circularly permuted protein retains the ability to bind DNA when complexed with a guide RNA (gRNA). Such CP variants of Cas9 can be used with the mtDNA base editors described herein.

### Cytidine Deaminase

As used herein, a “cytidine deaminase” encoded by the CDA gene is an enzyme that catalyzes the removal of an amine group from cytidine (i.e., the base cytosine when attached to a ribose ring) to uridine (C to U) and deoxycytidine to deoxyuridine (C to U). A non-limiting example of a cytidine deaminase is APOBEC1 (“apolipoprotein B mRNA editing enzyme, catalytic polypeptide 1”). Another example is AID (“activation-induced cytidine deaminase”). Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytidine deaminase will cause the insertion of “A” instead of a “G” during cellular repair and/or replication processes. Since the adenine “A” pairs with thymine “T”, the cytidine deaminase in coordination with DNA replication causes the conversion of an C-G pairing to a T-A pairing in the double-stranded DNA molecule.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### Deaminase

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. In other embodiments, the deaminase is a cytidine (or cytosine) deaminase, which catalyzes the hydrolytic deamination of cytidine or cytosine. In preferred aspects, the deaminase is a double-stranded DNA deaminase, or is modified, evolved, or otherwise altered to be able to utilize double-strand DNA as a substrate for deamination.

The deaminase embraces the DddA domains described herein, and defined below. The DddA is a type of deaminase, but where the activity of the deaminase is against double-stranded DNA, rather than single-stranded DNA, which is the case for deaminases prior to the present disclosure.

The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

### DNA Editing Efficiency

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

### Downstream

As used herein, the terms “upstream” and “downstream” are relative terms that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

In another example, the mtDNA BEs contemplated herein can comprise a pair of fusion proteins wherein a first fusion protein binds upstream of a target nucleobase pair target of deamination, and a second fusion protein binds just downstream of the target nucleobase pair that is being targeted for deamination. The pair of fusion proteins each comprise a pDNAbp (e.g., a Cas9 domain, a mitoTALE, or a mitoZFP) which bind to a target site on either side of the targeted nucleobase pair. Each of the pDNAbps of each fusion protein are each fused to a DddA half portion (e.g., an N-terminal half and a C-terminal half of a DddA which is divided into two inactive fragments at a split site), which become co-localized at the target nucleobase pair upon binding of the pDNAbp domains at their respective upstream and downstream sites.

### DddA

The term “double-stranded DNA deaminase domain” or “DddA” (or equivalently, DddE) refers to a protein which catalyzes a deamination of a target nucleotide (e.g., C, A, G, C) in a double-stranded DNA molecule. Reference to DddA and double-stranded DNA deaminase are equivalent. In one embodiment, the DddA deaminates a cytidine. Deamination of cytidine, results in a uracil (or deoxyuracil in the case of deoxycytidine), and through replication and/or repair processes, converts the original C:G base pair to a T:A base pair. This change can also be referred to as a “C-to-T” edit because the C of the C:G pair is converted to a T of T:A pair. DddA, when expressed naturally, can be toxic to biological systems. While the mechanism of action is not clearly documented, one rationale for the observed toxicity is DddA's activity may cause indiscriminant deamination of cytidine in vivo on double-stranded target DNA (e.g., the cellular genome). Such indiscriminant deaminations may provoke celluar repair responses, including, but not limited to, degradation of genomic DNA.

### Effective Amount

The term “effective amount,” as used herein, refers to an amount of a biologically active agent that is sufficient to elicit a desired biological response. For example, in some embodiments, an effective amount of any of the fusion proteins as described herein, or compositions thereof, may refer to the amount of the fusion proteins sufficient to edit a target nucleotide sequence (e.g., mtDNA). In some embodiments, an effective amount of any of the fusion proteins as described herein, or compositions thereof (e.g., a fusion protein comprising a first mitoTALE or another pDNAbp and a first portion of a DddA, a second fusion protein comprising a second mitoTALE or another pDNAbp and a second portion of a DddA) that is sufficient to induce editing of a target nucleotide, which is proximal to a target nucleic acid sequence specifically bound and edited by the fusion protein (e.g., by the first or second mitoTALE). As will be appreciated by the skilled artisan, the effective amount of an agent (e.g., a fusion protein, a second fusion protein), may vary depending on various factors as, for example, on the desired biological response on the specific allele, genome, or target site to be edited, on the cell or tissue being targeted, and on the agent being used.

### Fusion Protein

The term “fusion protein” as used herein refers to a hybrid polypeptide which comprises protein domains from at least two different proteins (e.g., a first mitoTALE, a first portion of a DddA, a second mitoTALE, a second portion of a DddA). One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding site (e.g., a first or second mitoTALE) and a catalytic domain of a nucleic-acid editing protein (e.g., a first or second portion of a DddA). Another example includes a mitoTALE to a DddA or portion thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

### Guide Nucleic Acid

In embodiments involving mtDNA base editors that comprise Cas9 domains as the pDNAbp component, the Cas9 domain requires a guide RNA (or more generically, a guide nucleic acid) to program the binding of the Cas9 to a target site. The term “guide nucleic acid” or “napDNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system.

Guide RNA is a particular type of guide nucleic acid which is mostly commonly associated with a Cas protein of a CRISPR-Cas9 and which associates with Cas9, directing the Cas9 protein to a specific sequence in a DNA molecule that includes complementarity to protospace sequence of the guide RNA. As used herein, a “guide RNA” refers to a synthetic fusion of the endogenous bacterial crRNA and tracrRNA that provides both targeting specificity and scaffolding and/or binding ability for Cas9 nuclease to a target DNA. This synthetic fusion does not exist in nature and is also commonly referred to as an sgRNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein.

Guide RNA (“gRNA”)

In embodiments involving pDNAbp/DddA base editors that comprise Cas9 domains as the pDNAbp component, the Cas9 domain requires a guide RNA (or more generically, a guide nucleic acid) to program the binding of the Cas9 to a target site. As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to protospace sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein.

Guide RNAs may comprise various structural elements that include, but are not limited to (a) a spacer sequence—the sequence in the guide RNA (having ˜20 nts in length) which binds to a complementary strand of the target DNA (and has the same sequence as the protospacer of the DNA) and (b) a gRNA core (or gRNA scaffold or backbone sequence)-refers to the sequence within the gRNA that is responsible for Cas9 binding, it does not include the ˜20 bp spacer sequence that is used to guide Cas9 to target DNA.

### Guide RNA Target Sequence

As used herein, the “guide RNA target sequence” refers to the ˜20 nucleotides that are complementary to the protospacer sequence in the PAM strand. The target sequence is the sequence that anneals to or is targeted by the spacer sequence of the guide RNA. The spacer sequence of the guide RNA and the protospacer have the same sequence (except the spacer sequence is RNA and the protospacer is DNA).

### Guide RNA Scaffold Sequence

As used herein, the “guide RNA scaffold sequence” refers to the sequence within the gRNA that is responsible for Cas9 binding, it does not include the 20 bp spacer/targeting sequence that is used to guide Cas9 to target DNA.

### Host Cell

The term “host cell,” as used herein, refers to a cell that can host, replicate, and transfer a phage vector useful for a continuous evolution process as provided herein. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect. In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect. The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

### Inteins and Split-Inteins

In some embodiments, the mtDNA base editors or the polypeptides that comprise the mtDNA base editors (e.g., the pDNAbps and DddA) may be engineered to include intein and/or split-intein amino acid sequences.

As used herein, the term “intein” refers to auto-processing polypeptide domains found in organisms from all domains of life. An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. This process is also known as cis-protein splicing, as opposed to the natural process of trans-protein splicing with “split inteins.”

Split inteins are a sub-category of inteins. Unlike the more common contiguous inteins, split inteins are transcribed and translated as two separate polypeptides, the N-intein and C-intein, each fused to one extein. Upon translation, the intein fragments spontaneously and non-covalently assemble into the canonical intein structure to carry out protein splicing in trans.

Inteins and split inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

As used herein, the term “protein splicing” refers to a process in which an interior region of a precursor protein (an intein) is excised and the flanking regions of the protein (exteins) are ligated to form the mature protein. This natural process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., Davis, E. O., Dean, G. E., Gimble, F. S., Jack, W. E., Neff, N., Noren, C. J., Thomer, J., Belfort, M. Nucleic Acids Research 1994, 22, 1127-1127). The resulting proteins are linked, however, not expressed as separate proteins. Protein splicing may also be conducted in trans with split inteins expressed on separate polypeptides spontaneously combine to form a single intein which then undergoes the protein splicing process to join to separate proteins.

The elucidation of the mechanism of protein splicing has led to a number of intein-based applications (Comb, et al., U.S. Pat. No. 5,496,714; Comb, et al., U.S. Pat. No. 5,834,247; Camarero and Muir, J. Amer. Chem. Soc., 121:5597-5598 (1999); Chong, et al., Gene, 192:271-281 (1997), Chong, et al., Nucleic Acids Res., 26:5109-5115 (1998); Chong, et al., J. Biol. Chem., 273:10567-10577 (1998); Cotton, et al. J. Am. Chem. Soc., 121:1100-1101 (1999); Evans, et al., J. Biol. Chem., 274:18359-18363 (1999); Evans, et al., J. Biol. Chem., 274:3923-3926 (1999); Evans, et al., Protein Sci., 7:2256-2264 (1998); Evans, et al., J. Biol. Chem., 275:9091-9094 (2000); Iwai and Pluckthun, FEBS Lett. 459:166-172 (1999); Mathys, et al., Gene, 231:1-13 (1999); Mills, et al., Proc. Natl. Acad. Sci. USA 95:3543-3548 (1998); Muir, et al., Proc. Natl. Acad. Sci. USA 95:6705-6710 (1998); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999); Severinov and Muir, J. Biol. Chem., 273:16205-16209 (1998); Shingledecker, et al., Gene, 207:187-195 (1998); Southworth, et al., EMBO J. 17:918-926 (1998); Southworth, et al., Biotechniques, 27:110-120 (1999); Wood, et al., Nat. Biotechnol., 17:889-892 (1999); Wu, et al., Proc. Natl. Acad. Sci. USA 95:9226-9231 (1998a); Wu, et al., Biochim Biophys Acta 1387:422-432 (1998b); Xu, et al., Proc. Natl. Acad. Sci. USA 96:388-393 (1999); Yamazaki, et al., J. Am. Chem. Soc., 120:5591-5592 (1998)). Each reference is incorporated herein by reference.

### Lentiviral Vectors

Lentiviral vectors are derived from human immunodeficiency virus-1 (HIV-1). The lentiviral genome consists of single-stranded RNA that is reverse-transcribed into DNA and then integrated into the host cell genome. Lentiviruses can infect both dividing and non-dividing cells, making them attractive tools for gene therapy.

The lentiviral genome is around 9 kb in length and contains three major structural genes: gag, pol, and env. The gag gene is translated into three viral core proteins: 1) matrix (MA) proteins, which are necessary for virion assembly and infection of non-dividing cells; 2) capsid (CA) proteins, which form the hydrophobic core of the virion; and 3) nucleocapsid (NC) proteins, which protect the viral genome by coating and associating tightly with the RNA. The pol gene encodes for the viral protease, reverse transcriptase, and integrase enzymes which are essential for viral replication. The env gene encodes for the viral surface glycoproteins, which are essential for virus entry into the host cell by enabling binding to cellular receptors and fusion with cellular membranes. In some embodiments, the viral glycoprotein is derived from vesicular stomatitis virus (VSV-G). The viral genome also contains regulatory genes, including tat and rev. Tat encodes transactivators critical for activating viral transcription, while rev encodes a protein that regulates the splicing and export of viral transcripts. Tat and rev are the first proteins synthesized following viral integration and are required to accelerate production of viral mRNAs.

To improve the safety of lentivirus, the components necessary for viral production are split across multiple vectors. In some embodiments, the disclosure relates to delivery of a heterologous gene (e.g., transgene) via a recombinant lentiviral transfer vector encoding one or more transgenes of interest flanked by long terminal repeat (LTR) sequences. These LTRs are identical nucleotide sequences that are repeated hundreds or thousands of times and facilitate the integration of the transfer plasmid sequences into the host cell genome. Methods of the current disclosure also describe one or more accessory plasmids. These accessory plasmids may include one or more lentiviral packaging plasmids, which encode the pol and rev genes that are necessary for the replication, splicing, and export of viral particles. The accessory plasmids may also include a lentiviral envelope plasmid, which encodes the genes necessary for producing the viral glycoproteins which will allow the viral particle to fuse with the host cell.

### Ligand-Dependent Intein

In some embodiments, the mtDNA base editors or the polypeptides that comprise the mtDNA base editors (e.g., the pDNAbps and DddA) may be engineered to include ligand-dependent inteins.

The term “ligand-dependent intein,” as used herein refers to an intein that comprises a ligand-binding domain. Typically, the ligand-binding domain is inserted into the amino acid sequence of the intein, resulting in a structure intein (N)—ligand-binding domain—intein (C). Typically, ligand-dependent inteins exhibit no or only minimal protein splicing activity in the absence of an appropriate ligand, and a marked increase of protein splicing activity in the presence of the ligand. In some embodiments, the ligand-dependent intein does not exhibit observable splicing activity in the absence of ligand but does exhibit splicing activity in the presence of the ligand. In some embodiments, the ligand-dependent intein exhibits an observable protein splicing activity in the absence of the ligand, and a protein splicing activity in the presence of an appropriate ligand that is at least 5 times, at least 10 times, at least 50 times, at least 100 times, at least 150 times, at least 200 times, at least 250 times, at least 500 times, at least 1000 times, at least 1500 times, at least 2000 times, at least 2500 times, at least 5000 times, at least 10000 times, at least 20000 times, at least 25000 times, at least 50000 times, at least 100000 times, at least 500000 times, or at least 1000000 times greater than the activity observed in the absence of the ligand. In some embodiments, the increase in activity is dose dependent over at least 1 order of magnitude, at least 2 orders of magnitude, at least 3 orders of magnitude, at least 4 orders of magnitude, or at least 5 orders of magnitude, allowing for fine-tuning of intein activity by adjusting the concentration of the ligand. Suitable ligand-dependent inteins are known in the art, and in include those provided below and those described in published U.S. Patent Application U.S. 2014/0065711 A1; Mootz et al., “Protein splicing triggered by a small molecule.” J. Am. Chem. Soc. 2002; 124, 9044-9045; Mootz et al., “Conditional protein splicing: a new tool to control protein structure and function in vitro and in vivo.” J. Am. Chem. Soc. 2003; 125, 10561-10569; Buskirk et al., Proc. Natt. Acad. Sci. USA. 2004; 101, 10505-10510); Skretas & Wood, “Regulation of protein activity with small-molecule-controlled inteins.” Protein Sci. 2005; 14, 523-532; Schwartz, et al., “Post-translational enzyme activation in an animal via optimized conditional protein splicing.” Nat. Chem. Biol. 2007; 3, 50-54; Peck et al., Chem. Biol. 2011; 18 (5), 619-630; the entire contents of each are hereby incorporated by reference. Exemplary sequences are as follows:

| | | SEQ |
|---|---|---|
| NAME | SEQUENCE OF LIGAND-DEPENDENT INTEIN | ID NO: |
| 2-4 | CLAEGTRIFDPVTGTTHRIEDVVDGRKPIHVVAAAKDGTLLARPVVSWFDQGTRDVI | 17 |
| INTEIN: | GLRIAGGAIVWATPDHKVLTEYGWRAAGELRKGDRVAGPGGSGNSLALSLTADQMVS | |
| | ALLDAEPPILYSEYDPTSPFSEASMMGLLINLADRELVHMINWAKRVPGFVDLTLHD | |
| | QAHLLECAWLEILMIGLVWRSMEHPGKLLFAPNLLLDRNQGKCVEGMVEIFDMLLAT | |
| | SSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLD | |
| | AHRLHAGGSGASRVQAFADALDDKFLHDMLAEELRYSVIREVLPTRRARTFDLEVEE | |
| | LHTLVAEGVVVHNC | |
| 3-2 | CLAEGTRIFDPVTGTTHRIEDVVDGRKPIHVVAVAKDGTLLARPVVSWFDQGTRDVI | 18 |
| INTEIN | GLRIAGGAIVWATPDHKVLTEYGWRAAGELRKGDRVAGPGGSGNSLALSLTADQMVS | |
| | ALLDAEPPILYSEYDPTSPFSEASMMGLLTNLADRELVHMINWAKRVPGFVDLTLHD | |
| | QAHLLECAWLEILMIGLVWRSMEHPGKLLFAPNLLLDRNQGKCVEGMVEIFDMLLAT | |
| | SSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYTNVVPLYDLLLEMLD | |
| | AHRLHAGGSGASRVQAFADALDDKFLHDMLAEELRYSVIREVLPTRRARTFDLEVEE | |
| | LHTLVAEGVVVHNC | |
| 30R3-1 | CLAEGTRIFDPVIGTTHRIEDVVDGRKPIHVVAAAKDGTLLARPVVSWFDQGTRDVI | 19 |
| INTEIN | GLRIAGGATVWATPDHKVLTEYGWRAAGELRKGDRVAGPGGSGNSLALSLTADQMVS | |
| | ALLDAEPPIPYSEYDPTSPFSEASMMGLLINLADRELVHMINWAKRVPGFVDLTLHD | |
| | QAHLLECAWLEILMIGLVWRSMEHPGKLLFAPNLLLDRNQGKCVEGMVEIFDMLLAT | |
| | SSRFRMMNLQGEEFVCLKSIILLNSGVYTFLSSILKSLEEKDHIHRALDKITDTLIH | |
| | LMAKAGLTLQQQHQRLAQLLLILSHIRHMSNKGMEHLYSMKYKNVVPLYDLLLEMLD | |
| | AHRLHAGGSGASRVQAFADALDDKFLHDMLAEGLRYSVIREVLPTRRARTFDLEVEE | |
| | LHTLVAEGVVVHNC | |
| 30R3-2 | CLAEGTRIFDPVTGTTHRIEDVVDGRKPIHVVAAAKDGTLLARPVVSWFDQGTRDVI | 20 |
| INTEIN | GLRIAGGATVWATPDHKVLTEYGWRAAGELRKGDRVAGPGGSGNSLALSLTADQMVS | |
| | ALLDAEPPILYSEYDPTSPFSEASMMGLLTNLADRELVHMINWAKRVPGFVDLTLHD | |
| | QAHLLECAWLEILMIGLVWRSMEHPGKLLFAPNLLLDRNQGKCVEGMVEIFDMLLAT | |
| | SSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLD | |
| | AHRLHAGGSGASRVQAFADALDDKFLHDMLAEELRYSVIREVLPTRRARTFDLEVEE | |
| | LHTLVAEGVVVHNC | |
| 30R3-3 | CLAEGTRIFDPVTGTTHRIEDVVDGRKPIHVVAAAKDGTLLARPVVSWFDQGTRDVI | 21 |
| INTEIN | GLRIAGGATVWATPDHKVLTEYGWRAAGELRKGDRVAGPGGSGNSLALSLTADQMVS | |
| | ALLDAEPPIPYSEYDPTSPFSEASMMGLLTNLADRELVHMINWAKRVPGFVDLTLHD | |
| | QAHLLECAWLEILMIGLVWRSMEHPGKLLFAPNLLLDRNQGKCVEGMVEIFDMLLAT | |
| | SSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLD | |
| | AHRLHAGGSGASRVQAFADALDDKFLHDMLAEELRYSVIREVLPTRRARTFDLEVEE | |
| | LHTLVAEGVVVHNC | |
| 37R3-1 | CLAEGTRIFDPVTGTTHRIEDVVDGRKPIHVVAAAKDGTLLARPVVSWFDQGTRDVI | 22 |
| INTEIN | GLRIAGGATVWATPDHKVLTEYGWRAAGELRKGDRVAGPGGSGNSLALSLTADQMVS | |
| | ALLDAEPPILYSEYNPTSPFSEASMMGLLINLADRELVHMINWAKRVPGFVDLTLHD | |
| | QAHLLERAWLEILMIGLVWRSMEHPGKLLFAPNLLLDRNQGKCVEGMVEIFDMLLAT | |
| | SSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLD | |
| | AHRLHAGGSGASRVQAFADALDDKFLHDMLAEGLRYSVIREVLPTRRARTFDLEVEE | |
| | LHTLVAEGVVVHNC | |
| 37R3-2 | CLAEGTRIFDPVTGTTHRIEDVVDGRKPIHVVAAAKDGILLARPVVSWFDQGTRDVI | 23 |
| INTEIN | GLRIAGGAIVWATPDHKVLTEYGWRAAGELRKGDRVAGPGGSGNSLALSLTADQMVS | |
| | ALLDAEPPILYSEYDPTSPFSEASMMGLLINLADRELVHMINWAKRVPGFVDLILHD | |
| | QAHLLERAWLEILMIGLVWRSMEHPGKLLFAPNLLLDRNQGKCVEGMVEIFDMLLAT | |
| | SSRFRMMNLQGEEFVCLKSIILLNSGVYTFLSSILKSLEEKDHIHRALDKITDTLIH | |
| | LMAKAGLTLQQQHQRLAQLLLILSHIRHMSNKGMEHLYSMKYKNVVPLYDLLLEMLD | |
| | AHRLHAGGSGASRVQAFADALDDKFLHDMLAEGLRYSVIREVLPTRRARTFDLEVEE | |
| | LHTLVAEGVVVHNC | |
| 37R3-3 | CLAEGTRIFDPVTGTTHRIEDVVDGRKPIHVVAVAKDGTLLARPVVSWFDQGTRDVI | 24 |
| INTEIN | GLRIAGGATVWATPDHKVLTEYGWRAAGELRKGDRVAGPGGSGNSLALSLTADQMVS | |
| | ALLDAEPPILYSEYDPTSPFSEASMMGLLTNLADRELVHMINWAKRVPGFVDLTLHD | |
| | QAHLLERAWLEILMIGLVWRSMEHPGKLLFAPNLLLDRNQGKCVEGMVEIFDMLLAT | |
| | SSRFRMMNLQGEEFVCLKSIILLNSGVYTFLSSILKSLEEKDHIHRALDKITDTLIH | |
| | LMAKAGLTLQQQHQRLAQLLLILSHIRHMSNKGMEHLYSMKYKNVVPLYDLLLEMLD | |
| | AHRLHAGGSGASRVQAFADALDDKFLHDMLAEELRYSVIREVLPTRRARTFDLEVEE | |
| | LHTLVAEGVVVHNC | |

### Linker

In various embodiments, the herein disclosed fusion proteins (e.g., the mtDNA base editors) or the polypeptides that comprise the mtDNA base editors (e.g., the pDNAbps and DddA) may be engineered to include one or more linker sequences that join two or more polypeptides (e.g., a pDNAbp and a DddA half) to one another.

The term “linker,” as used herein, refers to a molecule linking two other molecules or moieties. The linker can be an amino acid sequence in the case of a linker joining two fusion proteins. For example, a first or second mitoTALE can be fused to a first or second portion of a DddA, by an amino acid linker sequence. The linker can also be a nucleotide sequence in the case of joining two nucleotide sequences together. In other embodiments, the linker is an organic molecule, group, polymer, or chemical moiety. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer linkers are also contemplated.

mitoTALE

In various embodiments, the mtDNA base editors embrace fusion proteins comprising a DddA (or inactive fragment thereof) and a mitoTALE domain. As used herein, a “mitoTALE” protein or domain refers to a modified TALE protein that can be designed to localize to the mitochondria. In one embodiment, a mitoTALE comprises a TALE domain fused to a mitochondrial targeting sequences (MTS). In another embodiment, a mitoTALE comprises a TALE domain fused to an MTS in place of the endogenous LS (localization signal) of the TALE, or into the repeat variable diresidue (RVD) of the TALE. MTS domains can include, but are not limited to, SOD2, Cox8a, bipartitie nuclear localization signals (BPNLS), zmLOC100282174 MLS), which are disclosed herein.

Transcription activator-like effector proteins (TALE proteins) are class of naturally occurring DNA binding proteins which bind specific promoter sequences and which can activate the expression of genes. TALE proteins can be engineered to recognize a desired DNA sequence. TALEs have a modular DNA-binding domain (DBD) consisting of repetitive sequences of amino acids with each repeat region comprising of 34 amino acids. The two amino acids at residue positions 12 and 13 of each repeat region determine the nucleotide specificity of the TALE. This pair of residues is referred to as the repeat variable diresidue (RVD). A final region, known as the half-repeat, is typically truncated to 20 amino acids. Using these factors, one of ordinary skill in the art can sythesize sequence-specific synthetic TALEs, which target user defined nucleotide sequences. See Garg A.; Lohmueller J. J.; Silver P. A.; Armel T. Z. (2012), “Engineering synthetic TAL effectors with orthogonal target sites,” Nucleic Acids Res. 40, 7584-7595, which is incorporated herein by reference. Further reference to designing sequence specific TALEs can be found in Carlson et al., “Targeting DNA with fingers and TALENs,” Mol. Ther. Nucleic Acids, 2012, 1, e3.10.1038/mtna.2011, which is incorporated herein by reference. For example, the C-terminus typically contains a localization signal (LS), which directs a TALE to the particular cellular component (e.g., mitochondria), as well as a functional domain that modulates transcription, such as an acidic activation domain (AD). The endogenous LS can be replaced by an organism-specific localization signal, such as a specific MLS to localize the TALE to the mitochondria. For example, an LS derived from the simian virus 40 large T-antigen can be used in mammalian cells.

mitoZFP

In various embodiments, the mtDNA base editors embrace fusion proteins comprising a DddA (or inactive fragment thereof) and a mitoZFP domain.

A “zinc finger DNA binding protein” or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein can be abbreviated as zinc finger protein or ZFP. A “mitoZFP” refers to a zinc finger DNA binding protein that has been modified to comprise one or more mitochondral targeting sequences (MTS).

Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; 6,534,261; and 6,785,613; see, also WO 98/53058; WO 98/53059; WO 98/53060; WO 02/016536 and WO 03/016496; and U.S. Pat. Nos. 6,746,838; 6,866,997; and 7,030,215, each of which are incorporated herein by reference.

Zinc-finger nucleases (“ZFNs”) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes.

The DNA-binding domains of individual ZFNs typically contain between three and six individual zinc finger repeats and can each recognize between 9 and 18 basepairs. If the zinc finger domains are perfectly specific for their intended target site then even a pair of 3-finger ZFNs that recognize a total of 18 basepairs can, in theory, target a single locus in a mammalian genome. The most straightforward method to generate new zinc-finger arrays is to combine smaller zinc-finger “modules” of known specificity. The most common modular assembly process involves combining three separate zinc fingers that can each recognize a 3 basepair DNA sequence to generate a 3-finger array that can recognize a 9 basepair target site.

### Mitochondrial Targeting Sequence (MTS)

In various embodiments, the mtDNA base editors or the polypeptides that comprise the mtDNA base editors (e.g., the pDNAbps and DddA) may be engineered to include one or more mitochondrial targeting sequences (MTS) (or mitochondrial localization sequence (MLS)) which facilitate that translocation of a polypeptide into the mitochondria. MTS are known in the art and exemplary sequences are provided herein. In general MTSs are short peptide sequences (about 3-70 amino acids long) that direct a newly synthesized protein to the mitochondria within a cell. It is usually found at the N-terminus and consists of an alternating pattern of hydrophobic and positively charged amino acids to form what is called an amphipathic helix. Mitochondrial localization sequences can contain additional signals that subsequently target the protein to different regions of the mitochondria, such as the mitochondrial matrix. One exemplary mitochondrial localization sequence is the mitochondrial localization sequence derived from Cox8, a mitochondrial cytochrome c oxidase subunit VIII. In embodiments, a mitochondrial localization sequence derived from Cox8 includes the amino acid sequence: MSVLTPLLLRGLTGSARRLPVPRAKIHSL (SEQ ID NO: 299). In the embodiments, the mitochondrial localization sequence derived from Cox8 includes an amino acid sequence that is about 50%, 55%, 60%, 65%, 70%, 75%, 80%, 85%, 90% or 95% identity to SEQ ID NO: 299.

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, 0(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### Mutation

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which are mutations that reduce or abolish a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively, the mutation could lead to overexpression of one or more genes involved in control of the cell cycle, thus leading to uncontrolled cell division and hence to cancer. Because of their nature, gain-of-function mutations are usually dominant.

napDNAbp

In various embodiments, the mtDNA base editors may comprise pDNAbps which are nucleic acid programmable. The term “napDNAb” which stand for “nucleic acid programmable DNA binding protein” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each are herein incorporated by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al.., Proc. Natl. Acad. Sci. U.S.A. 98:4658-4663(2001); “CRISPR RNA maturation by trans-encoded small RNA and host factor RNase III.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

### Nickase

The term “nickase” refers to a napDNAbp having only a single nuclease activity that cuts only one strand of a target DNA, rather than both strands. Thus, a nickase type napDNAbp does not leave a double-strand break.

### Nuclear Localization Signal

In various embodiments, the mtDNA base editors or the polypeptides that comprise the mtDNA base editors (e.g., the pDNAbps and DddA) may be further engineered to include one or more nuclear localization signals.

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example more than 25, 25, 15, 12, 10, 8, 7, 6, 5, or 4 amino acids, but will preferably comprise at least a four to eight amino acid sequence known to function as a nuclear localization signal (NLS).

### Nucleic Acid Molecule

The term “nucleic acid molecule” as used herein, refers to RNA as well as single and/or double-stranded DNA. Nucleic acid molecules may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, 0(6)-methylguanine, and 2-thiocytidine); chemically modified bases; biologically modified bases (e.g. methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

### PACE

The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. PACE can be used, for instance, to evolve a deaminase (e.g., a cytidine or adenosine deaminase) which uses single strand DNA as a substrate to obtain a deaminase which is capable of using double-strand DNA as a substrate (e.g., DddA).

Programmable DNA Binding Protein (pDNAbp)

As used herein, the term “programmable DNA binding protein,” “pDNA binding protein,” “pDNA binding protein domain” or “pDNAbp” refers to any protein that localizes to and binds a specific target DNA nucleotide sequence (e.g. a gene locus of a genome). This term embraces RNA-programmable proteins, which associate (e.g. form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. The term also embraces proteins which bind directly to nucleotide sequence in an amino acid-programmable manner, e.g., zinc finger proteins and TALE proteins. Exemplary RNA-programmable proteins are CRISPR-Cas9 proteins, as well as Cas9 equivalents, homologs, orthologs, or paralogs, whether naturally occurring or non-naturally occurring (e.g. engineered or modified), and may include a Cas9 equivalent from any type of CRISPR system (e.g. type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353(6299), the contents of which are incorporated herein by reference.

### Promoter

The term “promoter” is recognized in the art as referring to a nucleic acid molecule with a sequence recognized by the cellular transcription machinery and able to initiate transcription of a downstream (i.e., closer to or toward the 3′ end of the nucleic acid strand) gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

### Protein, Peptide, and Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups {e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the njPAC-R7B Biochemical Nomenclature Commission. Nucleotides, likewise, may be referred to by their commonly accepted single-letter codes. The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

### RNA-Protein Recruitment System

In various embodiments, two separate protein domains (e.g., a pDNAbp and a DddA or a DddA N-terminal half and a DddA C-terminal half) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (aka “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a base editor, as well as to recruitment additional functionalities to a base editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplarly scenario a deaminase-MS2 fusion can recruit a Cas9-MCP fusion.

A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 25).

The amino acid sequence of the MCP or MS2cp is:

(SEQ ID NO: 26)
GSASNFTQFVLVDNGGTGDVTVAPSNFANGVAEWISSNSRSQAYKVT
CSVRQSSAQNRKYTIKVEVPKVATQTVGGEELPVAGWRSYLNMELTI
PIFATNSDCELIVKAMQGLLKDGNPIPSAIAANSGIY.

### Sense Strand

In genetics, a “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Split Site (e.g., of a DddA)

As used herein, the term “split site,” as in a split site of a DddA, refers to a specific peptide bond between any two immediately adjacent amino acid residues in the amino acid sequence of a DddA at which the complete DddA polypeptide is divided into two half portions, i.e., an N-terminal half portion and a C-terminal half portion. The N-terminal half portion of the DddA may be referred to as “DddA-N half” and the C-terminal half portion of the DddA may be referred to as the “DddA-C half.” Alternately, DddA-N half may be referred to as the “DddA-N fragment or portion” and the DddA-C half may be referred to as the “DddA-C fragment of portion.” Depending on the location of the split site, the DddA-N half and the DddA-C half may be the same or different size and/or sequence length. The term “half” does not connote the requirement that the DddA-N and DddA-C portions are identically half of the size and/or sequence length of a complete DddA, or that the split site is required to be at the mid point of the complete DddA polypeptide. To the contrary, and as noted above, the split site can be between any pair of residues in the DddA polypeptide, thereby giving rise to half portions which are unequal in size and/or sequence length. For clarity, as used herein, the term “half” when used in the context of a split molecule (e.g., protein, intein, delivery molecule, nucleic acid, etc.), shall not be interpreted to require, and shall not imply, that the size of the resulting portions (e.g., as “split” or broken into smaller portions) of the molecule are one-half (e.g., ½, 50%) of the original molecule. The term shall be interpreted to be illustriative of idea that they are portion(s) of a larger molecule that has been broken into smaller fragments (e.g., portions), but that when reconstituted may regain the activity of the molecule as a whole. Thus, by way of example, a half (e.g., portion) may be any portion of the molecule from which it is obtained (e.g., is less than 100% of the whole of the molecule), such that there is at least one additional portion formed (e.g., a second half, other half, second portion), which also is less than 100% of the whole of the molecule. It is important to note, that the molecule may be formed into additional portions (e.g., third, fourth, etc., halves (e.g., portions)), which is readily envisioned by using the term definition above, and such additional halves to not constitute a molecule larger than or in addition to the whole from which they were derived. Further, it should be noted that in the event there are more than two halves (e.g., two portions) formed from the splitting of a molecule it may only require two of the portions to reconstitute the activity of the molecule as a whole. By way of example, if an enzyme is split into three halves (e.g., three portions), wherein the catalytic domain of the enzyme possessing the enzymatic activity of interest is only split into two halves (e.g., two portions) only the two portions of the catalytic domain may be necessary to be used to carry out the activity of interest. Thus, when referring to using two halves, it is not necessary that the two halves, together, comprise 100% of the whole of the molecule from which they were derived. In certain embodiments, the split site is within a loop region of the DddA.

As used herein, reference to “splitting a DddA at a split site” embraces direct and indirect means for obtaining two half portions of a DddA. In one embodiment, splitting a DddA refers to the direct splitting a DddA polypeptide at a split site in the protein to obtain the DddA-N and DddA-C half portions. For example, the cleaving of a peptide bond between two adjacent amino acid residues at a split site may be achieved by enzymatic or chemical means. In another embodiment, a DddA may be split by engineering separate nucleic acid sequences, each encoding a different half portion of the DddA. Such methods can be used to obtain expression vectors for expressing the DddA half portions in a cell in order to reconstitute the DddA.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Target Site

The term “target site” refers to a sequence within a nucleic acid molecule (e.g., a mtDNA) that is edited by a mtDNA base editor disclosed herein. The target site further refers to the sequence within a nucleic acid molecule to which a complex of the mtDNA base editor binds. In cases wherein the pDNAbp of the mtDNA base editor is a Cas9 domain, typically, the target site is a sequence that includes the unique ˜20 bp target specified by the gRNA plus the genomic PAM sequence. CRISPR-Cas9 mechanisms recognize DNA targets that are complementary to a short CRISPR sgRNA sequence. The part of the sgRNA sequence that is complementary to the target sequence is known as a protospacer. In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand.

### Transition

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transitions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

### Transversion

As used herein, “transversions” refer to the interchange of purine nucleobases for pyrimidine nucleobases, or in the reverse and thus, involve the interchange of nucleobases with dissimilar shape. These changes involve T↔A, T↔G, C↔G, C↔A, A↔T, A↔C, G↔C, and G↔T. In the context of a double-strand DNA with Watson-Crick paired nucleobases, transversions refer to the following base pair exchanges: T:A↔A:T, T:A↔G:C, C:G↔G:C, C:G↔A:T, A:T↔T:A, A:T↔C:G, G:C↔C:G, and G:C↔T:A. The compositions and methods disclosed herein are capable of inducing one or more transversions in a target DNA molecule. The compositions and methods disclosed herein are also capable of inducing both transitions and transversion in the same target DNA molecule, as well as other nucleotide changes, including deletions and insertions.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Upstream

As used herein, the terms “upstream” and “downstream” are terms of relativety that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Uracil Glycosylase Inhibitor

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 27. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 27. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 27. In some embodiments, a UGI comprises an amino acid sequence homologous to the amino acid sequence set forth in SEQ ID NO: 27, or an amino acid sequence homologous to a fragment of the amino acid sequence set forth in SEQ ID NO: 27. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 27. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 27. In some embodiments, the UGI comprises the following amino acid sequence: MTNLSDIIEKETGKQLVIQESILMLPEEVEEVIGNKPESDILVHTAYDESTDENVMLLTSD APEYKPWALVIQDSNGENKIKML (SEQ ID NO: 27) (P14739|UNGI_BPPB2 Uracil-DNA glycosylase inhibitor), or the same sequence but without the N-terminal methionine.

Other UGI proteins may include those described in Example 6, as follows:

| | | SEQ |
|---|---|---|
| UGI | Sequence | ID NO: |
| Canonical | TNLSDIIEKETGKQLVIQESILMLPEEVE | 341 |
| UGI | EVIGNKPESDILVHTAYDESTDENVMLLT | |
| | SDAPEYKPWALVIQDSNGENKIKML | |
| UGI2 | MTLELQLKHYITNLFNLPKDEKWHCESIE | 445 |
| | EIADDILPDQYVRLGALSNKILQTYTYYS | |
| | DTLHESNIYPFILYYQKQLIAIGYIDENH | |
| | DMDFLYLHNTIMPLLDQRYLLTGGQ | |
| UGI3 | MNKNFDEVKADLRTVTGKKIEFKERLKNI | 446 |
| | LRVQMNQLGFEDSYMIQVQVSSDQEEWVE | |
| | CHENMSLSDFEVMYGNISGEIKRMTVVKY | |
| | EEANIEKLVELKFEYEYAKAHQEYIRAYT | |
| | KLMSNTLYGRKPSL | |
| UGI5 | MNEEKMHYRDAIKEVELTMMSLDSHFRTH | 447 |
| | KEFTDSYLLVLILEDVVGETRVEVSEGLT | |
| | FDEASYIIGGTSDNILNMHMINYCEKNRE | |
| | EIYKWLKVSRVNTFKSNYAKMLLNTAYGK | |
| | DLLKGVVK | |
| UGI7 | MNNHFMSIGRNCSKCNNVRLNEDFSKSEE | 556 |
| | ICNECFDKEERFVDSYTLIYITEDETGKR | |
| | FEAILENQTIEETEIIYGNIIDKIIVWNV | |
| | ILTM | |
| UGI12 | DGNEHWEVHPGLSLSDFEVVYGNNPHQIV | 376 |
| | KLRLDKEVGGSGGSMVQNDFIDSYTLCWL | |
| | LRDDSGGGGSMVQNDFIDSYTLCWLLRDD | |
| | DGNEHWEVHPGLSLSDFEVVYGNNPHQIV | |
| | KLRLDKEV | |

### Variant

In various embodiments, the mtDNA base editors or the polypeptides that comprise the mtDNA base editors (e.g., the pDNAbps and DddA) may be engineered as variants.

As used herein, the term “variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. The term also encompasses circular permutants, mutants, truncations, or domains of a reference sequence, and which display the same or substantially the same functional activity or activities as the reference sequence. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g. DddA).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a DddA protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene or characteristic as it occurs in nature as distinguished from mutant or variant forms.

These and other exemplary embodiments are described in more detail in the Detailed Description, Examples, and claims. The invention is not intended to be limited in any manner by the above exemplary embodiments.

### Detailed Description of Certain Embodiments

Each mammalian cell contains hundreds to thousands of copies of a circular mtDNA¹⁰. Homoplasmy refers to a state in which all mtDNA molecules are identical, while heteroplasmy refers to a state in which a cell contains a mixture of wild-type and mutant mtDNA. Current approaches to engineer mtDNA rely on DNA-binding proteins such as transcription activator-like effectors nucleases (mitoTALENs)¹¹⁻¹⁷ and zinc finger nucleases (mitoZFNs)¹⁸⁻²⁰ fused to mitochondrial targeting sequences to induce double-strand breaks (DSBs). Such proteins do not rely on nucleic acid programmability (e.g., such as with Cas9 domains). Linearized mtDNA is rapidly degraded,²¹⁻²³ resulting in heteroplasmic shifts to favor uncut mtDNA genomes. As a candidate therapy however, this approach cannot be applied to homoplasmic mtDNA mutations²⁴ since destroying all mtDNA copies is presumed to be harmful.^{22,25} In addition, using DSBs to eliminate heteroplasmic mtDNA mutations, which tend to be functionally recessive,²⁶ implicitly requires the edited cell to restore its wild-type mtDNA copy number. During this transient period of mtDNA repopulation, the loss of mtDNA copies could result in cellular toxicity.

The present disclosure relates in part to the inventors' discovery of a double-stranded DNA deaminase, referred to herein as “DddA,” and to its application in base editing of double-stranded nucleic acid molecules, and in particular, the editing of mitochondrial DNA.

Accordingly, the disclosure provides a novel platform of precision genome editing using a double-stranded DNA deaminase (DddA) and a programmable DNA binding protein (pDNAbp), such as a TALE domain, zinc finger binding domain, or a napDNAbp (e.g., Cas9), to target the deamination of a target base, which through cellular DNA repair and/or replication, is converted to a new base, thereby installing a base edit at a target site. In some embodiments, the deaminase activity is a cytidine deminase, which deaminates a cytidine, leading to a C-to-T edit at that site. In some other embodiments, that deaminase activity is an adenosine deminase, which deaminates an adenosine, leading to a A-to-G edit at that site. In various embodiments, the disclosure further relates to “split-constructs” and “split-delivery” of said constructs whereby to address the toxic nature of fully active DddA in cells (as discovered by the inventors), the DddA protein is “split” or otherwise divided into two or more DddA fragments which can be separately delivered, expressed, or otherwise provided to cells to avoid the toxicity of fully active DddA. Further, the DddA fragments may be delivered, expressed, or otherwise provided as separate fusion proteins to cells with programmable DNA binding proteins (e.g., zinc finger domains, TALE domains, or Cas9 domains) which are programmed to localize the DddA fragments to a target edit site, through the binding of the DNA binding proteins to DNA sites upstream and downstream of the target edit site. Once co-localized to the target edit site, the separately provided DddA fragments may associate (covalently or non-covalently) to reconstitute an active DddA protein with a double-stranded DNA deaminase activity. In certain embodiments where the objective is to base edit mitochondrial DNA targets, the programmable DNA binding proteins can be modified with one or more mitochondrial localization signals (MLS) so that the DddA-pDNAbp fusions are translocated into the mitochondria, thereby enabling them to act on mtDNA targets.

The inventors are believed to be the first to identify DddA, initially being discovered as a bacterial toxin. The inventors further conceived of the idea of splitting the DddA into two or more domains, which apart do not have a deaminase activity (and as such, lack toxicity), but which may be reconstituted to restore the deaminase activity of the protein. This allows the separate delivery DddA fragments to cells, or delivery of nucleic acid molecules expressing such DddA fragments to a cell, such that once present or expressed within a cell, DddA fragments may associate with one another By “associate” it is meant the two or more DddA fragments may come into contact with one another (e.g., in a cell) and form a functional DddA protein within a cell. The association of the two or more fragments may be through covalent interactions or non-covalent interactions. In addition, the DddA domains may be fused or otherwise non-covalently linked to a programmable DNA binding protein, such as a Cas9 domain or other napDNAbp domain, zinc finger domain or protein (ZF, ZFD, or ZFP), or a transcription activator-like effector protein (TALE), which allows for the co-localization of the two or more DddA fragments to a particular desired site in a target nucleic acid molecule which is to be edited, such that when the DddA fragments are co-localized at the desired editing site, they reform a functional DddA that is capable deaminating a target site on a double-stranded DNA molecule. In certain embodiments, the programmable DNA binding proteins can be engineered to comprise one or more mitochondrial localization signals (MLS) such the DddA domains become translocated into the mitochondria, thereby providing a means by which to conduct base editing directly on the mitochondrial genome.

FIG. 1A is a schematic representation of a naturally occurring DddA, an interbacterial toxin discovered by the inventors which was found to catalyze deamination of cytidines within double-stranded DNA as a substrate. The inventors are believed to be the first to identify such a deaminase. However, in its naturally occurring form, the inventors discovered that DddA is toxic to cells. The inventors have conceived of the idea of using the DddA in the context of base editing to deaminate a nucleobase at a target edit site.

In the context of base editing, all previously described cytidine deaminases utilize single-stranded DNA as a substrate (e.g., the R-loop region of a Cas9-gRNA/dsDNA complex). Base editing in the context of mitochondrial DNA has not heretofore been possible due to the challenges of introducing and/or expressing the gRNA needed for a Cas9-based system into mitochondria. The inventors have recognized for the first time that the catalytic properties of DddA can be leveraged to conduct base editing directly on a double strand DNA substrate by separating the DddA into inactive portions, which when co-localized within a cell will become reconstituted as an active DddA. This avoids or at least minimizes the toxicity associated with delivering and/or expressing a fully active DddA in a cell.

For example, a DddA may be divided into two fragments at a “split site,” i.e., a peptide bond between two adjacent residues in the primary structure or sequence of a DddA. The split site may be positioned anywhere along the length of the DddA amino acid sequence, so long as the resulting fragments do not on their own possess a toxic property (which could be a complete or partial deaminase activity). In certain embodiments, the split site is located in a loop region of the DddA protein. In the embodiment shown in FIG. 1A, the arrows depict five possible split sites approximately equally spaced along the length of the DddA protein. The depicted embodiment further shows that the DddA was divided into two fragments at a split site located approximately in the middle of the DddA amino acid sequence. The DddA fragment lying to the left of the split site may be referred to as the “N-terminal DddA half” and the DddA fragment lying to the right of the split site may be referred to as the “C-terminal DddA half.” FIG. 1A identifies these fragments as “DddA half^{A}” and DddA half^{B},” respectively. Depending on the location of the split site, the N-terminal DddA half and the C-terminal DddA half could be the same size, approximately the same size, or very different sizes.

Accordingly, this disclosure provides compositions, kits, and methods of modifying double-stranded DNA (e.g., mitochondrial DNA or “mtDNA”) using genome editing strategies that comprise the use of a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in double-stranded DNA (e.g., mtDNA), rather than destroying the DNA (e.g., mtDNA) with double-strand breaks (DSBs). The present disclosure provides pDNAbp polypeptides, DddA polypeptides, fusion proteins comprising pDNAbp polypeptides and DddA polypeptides, nucleic acid molecules encoding the pDNAbp polypeptides, DddA polypeptides, and fusion proteins described herein, expression vectors comprising the nucleic acid molecules described herein, cells comprising the nucleic acid molecules, expression vectors, pDNAbp polypeptides, DddA polypeptides, and/or fusion proteins described herein, pharmaceutical compositions comprising the polypeptides, fusion proteins, nucleic acid molecules, vectors, or cells described herein, and kits comprising the polypeptides, fusion proteins, nucleic acid molecules, vectors, or cells described herein for modifying double-stranded DNA (e.g., mtDNA) by base editing.

Mitochondrial diseases (e.g., MELAS/Leigh syndrome and Leber's hereditary optic neuropathy) are diseases often resulting from errors or mutations in the mitochondrial DNA (mtDNA). In many cases, the mutated mtDNA co-exists with the wild-type mtDNA (mtDNA heteroplasmy). In such instances, residual wild type mtDNA can partially compensate for the mutation before biochemical and clinical manifestations occur. Multiple approaches to reduce the levels of mutant mtDNA have been tried. None of these approaches, however, have been successful in treating or correcting these abnormalities. The present disclosure, including the disclosed DddA/pDNAbp fusion proteins, nucleic acid molecules and vectors encoding same can be used to treat one or more mitochondrial diseases, which can include, but are not limited to: Alper's Disease, Autosomal Dominant Optic Atrophy (ADOA), Barth Syndrome, Carnitine Deficiency, Chronic Progressive External Ophthalmoplegia (CPEO), Co-Enzyme Q10 Deficiency, Creatine Deficiency Syndrome, Fatty Acid Oxidation Disorders, Friedreich's Ataxia, Kearns-Sayre Syndrome (KSS), Lactic Acidosis, Leber Hereditary Optic Neuropathy (LHON), Leigh Syndrome, MELAS, Mitochondrial Myopathy, Multiple Mitochondrial Dysfunction Syndrome, Primary Mitochondrial Myopathy, and TK2d, among others.

The present disclosure addresses many of the shortcomings of the exisiting technologies with a new precision mtDNA editing fusion protein and technique. The proposed technology permits the editing (e.g., deamination) of single, or multiple, nucleotides in the mtDNA allowing for the correction or modification of the nucleotide, and by extension the codon in which it is contained. In various embodiment, however, the present disclosure is not limited to editing mtDNA, but may also be used to target the editing of any double-stranded DNA in the cell, including the genomic DNA in the nucleus.

mtDNA BEs

Provided herein are base editor fusion proteins, vectors and nucleic acid molecule encoding base editor fusion proteins, kits, and methods of modifying mitochondrial DNA (mtDNA) using genome editing strategies that comprise the use of a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in mtDNA, rather than destroying the mtDNA with double-strand breaks (DSBs). In various embodiments, these polypeptides may be combined as fusion proteins referred to as “mtDNA base editors.” In various embodiments, that base editor fusion proteins may be provided as separate components, i.e., not as a fusion protein, but rather as separate pDNAbp and DddA domains which associate in the cell to target the desired edit site.

Also provided herein are base editor fusion proteins, vectors and nucleic acid molecule encoding base editor fusion proteins, kits, and methods of modifying any double-stranded DNA (e.g., genomic DNA) using genome editing strategies that comprise the use of a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in double-stranded DNA, rather than destroying the DNA with double-strand breaks (DSBs). In various embodiments, that base editor fusion proteins may be provided as separate components, i.e., not as a fusion protein, but rather as separate pDNAbp and DddA domains which associate in the cell to target the desired edit site.

The present disclosure provides mtDNA base editors, pDNAbp polypeptides, DddA polypeptides, nucleic acid molecules encoding the pDNAbp polypeptides, DddA polypeptides, and fusion proteins described herein, expression vectors comprising the nucleic acid molecules, cells comprising the nucleic acid molecules, expression vectors, and/or pDNAbp polypeptides, DddA polypeptides, or fusion proteins, pharmaceutical compositions comprising the polypeptides, fusion proteins, nucleic acid molecules, vectors, or the cells described herein, and kits comprising the polypeptides, fusion proteins, nucleic acid molecules, vectors, or the cells described herein for modifying mtDNA by base editing.

In some embodiments, the mtDNA base editors comprise a pDNAbp (e.g., a mitoTALE, mitoZFP, or a CRISPR/Cas) and a DddAs (or inactive fragment there). In other embodiments, the mtDNA base editors comprise separately expressed pDNAbps and DddAs, which may be co-localized at a desired target site through the use of split-intein sequences, RNA-protein recruitment systems, or other elements that facilitate the co-localization of separately expressed elements to a target site. In various other embodiments, the fusion proteins and/or the separately expressed pDNAbps and DddAs become translocated into the mitochondria. To effect translocation, the fusion proteins and/or the separately expressed pDNAbps and DddAs can comprise one or more mitochondrial targeting sequences (MTS).

In still other embodiments, the mtDNA base editors comprise a DddA domain which has been inactivated. In one embodiment, this inactivation can be achieved by engineering a whole DddA polypeptide into two or more fragments, each alone which is inactive and non-toxic to a cell. When the DddA inactive fragments become co-localization in the cell, e.g., inside the mitochondria, the fragments reconstitute the deaminase activity. The co-localization of the DddA fragments can be effectuated by fusing each DddA fragment to a separate pDNAbp that binds on either one side or the other of a target deamination site. For example, the embodiments depicted in FIG. 1A-1F show that splitting the DddA at a split site into two inactive DddA fragments (e.g., “DddA half^{A}” and “DddA half^{B}”) result in a non-toxic form of DddA. FIG. 1B shows that each of the inactive DddA fragments may be separately expressed as a fusion protein with a pDNAbp which binds to separate target sites on either side of a target deamination site. In FIG. 1B, these target sites are represented by “target site A” and “target site B”. By binding the pDNAbp domain of each of the fusion protein to their respective sites, the inactive DddA fragments become co-localized at the desired deamination site, thereby restoring the deaminase activity of the original DddA enzyme. FIGS. 1C, 1D, and 1E show this arrangement in the context of a mitoTALE, mitoZFP, and a Cas9/sgRNA complex as the pDNAbp domain of the mtDNA base editors.

In certain embodiments, the reconstituted activity of the co-localized two or more fragments can comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 99.9% of the deaminase activity of a wildtype DddA.

In terms of the spacing between the target site A and target site B from the site of deamination, any suitable spacing may be used, and which may be further dependent on the length of the linkers (if present) between the pDNAbp and the DddA domains, as well as the properties of the DddA domains. If the target nucleobase site (C on the deamination strand or a G:C nucleobase pair if referring to both strands) is assigned an arbitrary value of 0, then 3′-most position of target site A, in various embodiments, may be spaced at least 1 nucleotide upstream of the target G:C nucleobase pair, or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides upstream of the G:C nucleobase pair (or otherwise the target site of deamination). Likewise, the 3′-most position of target site B (i.e., which is on the opposite strand in this instance), may be spaced at least 1 nucleotide upstream of the target G:C nucleobase pair, or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides upstream of the G:C nucleobase pair (or otherwise the target site of deamination).

Looking at FIG. 1B-1F as a point of reference, it is also contemplated herein that target site A and target site B may be on the same strand of DNA. That is, the inactive DddA fragments may become co-localized at the desired site of deamination by using a pair of mtDNA base editor fusion proteins having pDNAbp components (e.g., mitoTALEs, mitoZFP, Cas9 domains) that both bind to target sites A and B on the same strand. In the case where a pair of mtDNA base editors bind to the same strand of DNA at separate target sites, the strand of DNA containing the target sites can be the same strand at the site of deamination, or the strand can be the opposite strand. So long as the inactive DddA fragments become co-localized at the intended site of deamination, the pair of base editor fusion proteins may bind to target sites on the same strands or opposite strands, and when binding to the same strand, the target sites can be the same or the opposite strand as the strand having the site of deamination.

In certain embodiments, the DddA can be separated into two fragments by dividing the DddA at a split site. A “split site” refers to a position between two adjacent amino acids (in a wildtype DddA amino acid sequence) that marks a point of division of a DddA. In certain embodiments, the DddA can have a least one split site, such that once divided at that split site, the DddA forms an N-terminal fragment and a C-terminal fragment. The N-terminal and C-terminal fragments can be the same or difference sizes (or lengths), wherein the size and/or polypeptide length depends on the location or position of the split site. As used herein, reference to a “fragment” of DddA (or any other polypeptide) can be referred equivalently as a “portion.” Thus, a DddA which is divided at a split site can form an N-terminal portion and a C-terminal portion. Preferably, the N-terminal fragment (or portion) and the C-terminal fragment (or portion) or DddA do not have a deaminase activity.

In various embodiments, a DddA may be split into two or more inactive fragments by directly cleaving the DddA at one or more split sites. Direct cleaving can be carried out by a protease (e.g., trypsin) or other enzyme or chemical reagent. In certain embodiments, such chemical cleavage reactions can be designed to be site-selective (e.g., Elashal and Raj, “Site-selective chemical cleavage of peptide bonds,” Chemical Communications, 2016, Vol. 52, pages 6304-6307, the contents of which are incorporated herein by reference.) In other embodiments, chemical cleavage reactions can be designed to be non-selective and/or occur in a random fashion.

In other embodiments, the two or more inactive DddA fragments can be engineered as separately expressed polypeptides. For instance, for a DddA having one split site, the N-terminal DddA fragment could be engineered from a first nucleotide sequence that encodes the N-terminal DddA fragment (which extends from the N-terminus of the DddA up to and including the residue on the amino-terminal side of the split site). In such an example, the C-terminal DddA fragment could be engineered from a second nucleotide sequence that encodes the C-terminal DddA fragment (which extends from the carboxy-terminus of the split site up to including the natural C-terminus of the DddA protein). The first and second nucleotide sequences could be on the same or different nucleotide molecules (e.g., the same or different expression vectors).

In various embodiments, that N-terminal portion of the DddA may be referred to as “DddA-N half” and the C-terminal portion of the DddA may be referred to as the “DddA-C half.” Reference to the term “half” does not connote the requirement that the DddA-N and DddA-C portions are identically half of the size and/or sequence length of a complete DddA, or that the split site is required to be at the mid point of the complete DddA polypeptide. To the contrary, and as noted above, the split site can be between any pair of residues in the DddA polypeptide, thereby giving rise to half portions which are unequal in size and/or sequence length. In certain embodiments, the split site is within a loop region of the DddA.

Accordingly, in one aspect, the disclosure relates to a pair of fusion proteins useful for making modifications to the sequence of mitochondrial DNA (e.g., mtDNA). The pair of fusion proteins, in some embodiments, can comprise a first fusion protein comprising a first pDNAbp (e.g., a mitoTALE, mitoZFP, or a CRISPR/Cas9) and a first portion or fragment of a DddA, and a second fusion protein comprising a second pDNAbp (e.g., mitoTALE, mitoZFP, or a CRISPR/Cas9) and a second portion or fragment of a DddA, such that the first and the second portions of the DddA reconstitute a DddA upon co-localization in a cell and/or mitochondria. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[pDNAbp]-[DddA half^{A}] and [pDNAbp]-[DddA half^{B}];
[DddA-half^{A}]-[pDNAbp] and [DddA-half^{B}]-[pDNAbp];
[pDNAbp]-[DddA half^{A}] and [DddA-half^{B}]-[pDNAbp]; or
[DddA-half^{A}]-[pDNAbp] and [pDNAbp]-[DddA half^{B}], wherein “A” or “B” can be the N-terminal or C-terminal half of DddA.

In another aspect, the disclosure relates to a pair of fusion proteins useful for making modifications to the sequence of mitochondrial DNA (e.g., mtDNA). The pair of fusion proteins can comprise a first fusion protein comprising a first mitoTALE and a first portion or fragment of a DddA, and a second fusion protein comprising a second mitoTALE and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA and the second portion of the DddA is C-terminal fragment of a Ddda. In other embodiments, the first portion of the DddA is a C-terminal fragment of a DddA and the second portion of the DddA is an N-terminal fragment of a DddA. In this aspect, the structure of the pair of fusion proteins can be, for example:
[mitoTALE]-[DddA half^{A}] and [mitoTALE]-[DddA half^{B}];
[DddA-half^{A}]-[pDNAbp] and [DddA-half^{B}]-[mitoTALE];
[mitoTALE]-[DddA half^{A}] and [DddA-half^{B}]-[mitoTALE]; or
[DddA-half^{A}]-[mitoTALE] and [mitoTALE]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first mitoZFP and a first portion or fragment of a DddA, and a second fusion protein comprising a second mitoZFP and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA and the second portion of the DddA is C-terminal fragment of a Ddda. In other embodiments, the first portion of the DddA is a C-terminal fragment of a DddA and the second portion of the DddA is an N-terminal fragment of a DddA. In this aspect, the structure of the pair of fusion proteins can be, for example:
[mitoZFP]-[DddA half^{A}] and [mitoZFP]-[DddA half^{B}];
[DddA-half^{A}]-[pDNAbp] and [DddA-half^{B}]-[mitoZFP];
[mitoZFP]-[DddA half^{A}] and [DddA-half^{B}]-[mitoZFP]; or
[DddA-half^{A}]-[mitoZFP] and [mitoZFP]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first Cas9 and a first portion or fragment of a DddA, and a second fusion protein comprising a second Cas9 and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA (i.e., “DddA half^{A}” as shown in FIGS. 1A-1E) and the second portion of the DddA is C-terminal fragment of a DddA (i.e., “DddA half^{B}” as shown in FIGS. 1A-1E). In other embodiments, the first portion of the DddA is an C-terminal fragment of a DddA and the second portion of the DddA is an N-terminal fragment of a DddA. In this aspect, the structure of the pair of fusion proteins can be, for example:
[Cas9]-[DddA half^{A}] and [Cas9]-[DddA half^{B}];
[DddA-half^{A}]-[Cas9] and [DddA-half^{B}]-[Cas9];
[Cas9]-[DddA half^{A}] and [DddA-half^{B}]-[Cas9]; or
[DddA-half^{A}]-[Cas9] and [Cas9]-[DddA half^{B}], wherein “A” or “B” can be the N-terminal or C-terminal half of DddA.

In each instance above of “]-[” can be in reference to a linker sequence.

In addition, the fusion proteins may have any suitable architecture, include any those depicted in FIGS. 1F.

In some embodiments, a first fusion protein comprises, a first mitochondrial transcription activator-like effector (mitoTALE) domain and a first portion of a DNA deaminase effector (DddA). In some embodiments, the first portion of the DddA comprises an N-terminal truncated DddA. In some embodiments, the first mitoTALE is configured to bind a first nucleic acid sequence proximal to a target nucleotide. In some embodiments, the first portion of a DddA is linked to the remainder of the first fusion protein by the C-terminus of the first portion of a DddA.

In one aspect, the present disclosure provides mitochondrial DNA editor fusion proteins for use in editing mitochondrial DNA. As used herein, these mitochondrial DNA editor fusion proteins may be referred to as “mtDNA editors” or “mtDNA editing systems.”

In various embodiments, the mtDNA editors described herein comprise (1) a programmable DNA binding protein (“pDNAbp”) (e.g., a mitoTALE domain, mitoZFP domain, or a CRISPR/Cas9 domain) and a double-stranded DNA deaminase domain, which is capable of carrying out a deamination of a nucleobase at a target site associated with the binding site of the programmable DNA binding protein (pDNAbp).

In some embodiments, the double-stranded DNA deaminase is split into two inactive half portions, with each half portion being fused to a programmable DNA binding protein that binds to a nucleotide sequence either upstream or downstream of a target edit site, and wherein once in the mitochondria, the two half portions (i.e., the N-terminal half and the C-terminal half) reassociate at the target edit site by the co-localization of the programmable DNA binding proteins to binding sites upstream and downstream of the target edit site to be acted on by the DNA deaminase. The reassociation of the two half portions of the double-stranded DNA deaminase restores the deaminase activity at the target edit site. In other embodiments, the double-stranded DNA deaminase can initially be set in an inactive state which can be induced when in the mitochondria. The double-stranded DNA deaminase is preferably delivered initially in an inactive form in order to avoid toxicity inherent with the protein. Any means to regulate the toxic properties of the double-stranded DNA deaminase until such time as the activity is desired to be activated (e.g., in the mitochondria) is contemplated.

The mtDNA base editors described herein contemplate fusion proteins comprising a mitoTALE and a DddA domain or fragment or portion thereof (e.g., an N-terminal or C-terminal fragment or portion of a DddA), and optionally the joining of the two by a linker. The application contemplates any suitable mitoTALE and a DddA domain to be combined in a single fusion protein. Examples of mitoTALEs and DddA domains are each defined herein.

In some embodiments, a first fusion protein comprises a first portion of a DddA fused (e.g., attached) to a first mitoTALE. In some embodiments, a second fusion protein comprises a second portion of a DddA fused (e.g., attached) to a second mitoTALE. In some embodiments, the first fusion protein comprises a first portion of a DddA linked to the remainder of the first fusion protein by the C-terminus of the first portion of a DddA. In some embodiments, a second fusion protein comprises a second portion of a DddA linked to the remainder of the second fusion protein by the C-terminus of the second portion of a DddA.

In some embodiments, the first fusion protein comprises a first mitoTALE to bind a target nucleic acid sequence proximal (as defined herein above) to the target nucleotide. In some embodiments, the second fusion protein comprises a mitoTALE to bind a target nucleic acid sequence proximal to the nucleotide complementary to the target nucleotide. In some embodiments, the first and second mitoTALEs are configured to bind proximally to the same target nucleotide (or nucleotide complementary thereto, as described herein above). In some embodiments, the first and second fusion proteins comprise mitoTALEs configured to bind first and second target nucleic acid sequences such that the first and second portions of DddA can dimerize (i.e., re-assemble) at or near the target nucleotide, such that re-assembled first and second portions of a DddA regain, at least partially, the native activity (e.g., deamination) of a full-length DddA. In some embodiments, the first and second fusion proteins comprise mitoTALEs configured to bind first and second target nucleic acid sequences such that that the first and second portions of a DddA can dimerize (i.e., re-assemble) at or near the target nucleotide, such that the target nucleotide is affected by activity of a re-assembled first and second portions of a DddA. Any suitable architecture of the fusion proteins comprising mitoTALEs are contemplated, and shows in FIG. 1F.

The mtDNA base editors described herein also contemplate fusion proteins comprising a mitoZF and a DddA domain or fragment or portion thereof (e.g., an N-terminal or C-terminal fragment or portion of a DddA), and optionally the joining of the two by a linker. The application contemplates any suitable mitoZF and a DddA domain to be combined in a single fusion protein. Examples of mitoZFs and DddA domains are each defined herein.

In some embodiments, a first fusion protein comprises a first portion of a DddA fused (e.g., attached) to a first mitoZF. In some embodiments, a second fusion protein comprises a second portion of a DddA fused (e.g., attached) to a second mitoZF. In some embodiments, the first fusion protein comprises a first portion of a DddA linked to the remainder of the first fusion protein by the C-terminus of the first portion of a DddA. In some embodiments, a second fusion protein comprises a second portion of a DddA linked to the remainder of the second fusion protein by the C-terminus of the second portion of a DddA.

In some embodiments, the first fusion protein comprises a first mitoZF to bind a target nucleic acid sequence proximal (as defined herein above) to the target nucleotide. In some embodiments, the second fusion protein comprises a mitoZF to bind a target nucleic acid sequence proximal to the nucleotide complementary to the target nucleotide. In some embodiments, the first and second mitoZFs are configured to bind proximally to the same target nucleotide (or nucleotide complementary thereto, as described herein above). In some embodiments, the first and second fusion proteins comprise mitoZFs configured to bind first and second target nucleic acid sequences such that the first and second portions of DddA can dimerize (i.e., re-assemble) at or near the target nucleotide, such that re-assembled first and second portions of a DddA regain, at least partially, the native activity (e.g., deamination) of a full-length DddA. In some embodiments, the first and second fusion proteins comprise mitoTALEs configured to bind first and second target nucleic acid sequences such that that the first and second portions of a DddA can dimerize (i.e., re-assemble) at or near the target nucleotide, such that the target nucleotide is affected by activity of a re-assembled first and second portions of a DddA. Any suitable architecture of the fusion proteins comprising mitoZFs are contemplated, and shows in FIG. 1F.

In some embodiments, the first fusion protein comprises the amino acid sequence of any one of SEQ ID NOs.: 360-375. In some embodiments, the first fusion protein comprises an amino acid sequence with 75% or greater percent identity (e.g., 80% or greater, 85% or greater, 90% or greater, 95% or greater, 96% or greater, 97% or greater, 98% or greater, 99% or greater, 99.5% or greater, 99.9% or greater percent identity) any one of SEQ ID NOs.: 360-375. In some embodiments, the second fusion protein comprises the amino acid sequence of any one of SEQ ID NOs.: 360-375. In some embodiments, the second fusion protein comprises an amino acid sequence with 75% or greater percent identity (e.g., 80% or greater, 85% or greater, 90% or greater, 95% or greater, 96% or greater, 97% or greater, 98% or greater, 99% or greater, 99.5% or greater, 99.9% or greater percent identity) to any one of SEQ ID NOs.: 360-375.

In some embodiments, the first and second fusion protein form pairs which result from the targeting of a similar target nucleotide, or which first and second portion of a DddA form a pair of portions which can re-assemble (e.g., dimerize) to form a protein with, at least partially, the activity of a full-length DddA (e.g., deamination). In some embodiments, the pair of fusion proteins comprise a first fusion protein comprising the first fusion protein of any one of and a second fusion protein comprising the second fusion protein wherein the first mitoTALE of the first fusion protein is configured to bind a first nucleic acid sequence proximal to a target nucleotide and the second mitoTALE of the second fusion protein is configured to bind a second nucleic acid sequence proximal to a nucleotide opposite the target nucleotide. In some embodiments, the first nucleic acid sequence is upstream of the target nucleotide and the second nucleic acid sequence is upstream of a nucleic acid of the complementary nucleotide of the target nucleotide. In some embodiments, the re-assembly (i.e., dimerization) of the first and second fusion proteins facilitate deamination of the target nucleotide.

mtDNA BEs Comprising mitoTALES

The mtDNA base editors described herein contemplate fusion proteins comprising a mitoTALE and a DddA domain or fragment or portion thereof (e.g., an N-terminal or C-terminal fragment or portion of a DddA), and optionally the joining of the two by a linker. The application contemplates any suitable mitoTALE and a DddA domain to be combined in a single fusion protein. Examples of mitoTALEs and DddA domains are each defined herein.

In some embodiments, the mtDNA base editors comprise DddA domains which are DdCBE, i.e., DddA which deaminates a C. Examples of general architecture of mtDNA base editors comprising DdCBEs and mitoTALEs and their amino acid and nucleotide sequences are as follows:

All right-side halves of DdCBEs have the general architecture of (from N- to C-terminus): COX8A MTS-3×FLAG-mitoTALE-2aa linker-DddAₜₒₓ half-4aa linker-1×-UGI-ATP5B 3′UTR

All left-side halves of DdCBEs have the general architecture of (from N- to C-terminus): SOD2 MTS-3×HA-mitoTALE-2aa linker-DddAₜₒₓ half-4aa linker-1×-UGI-SOD2 3′UTR

(A) SOD2 MTS
(SEQ ID NO: 298)
MLSRAVCGTSRQLAPVLGYLGSRQKHSLPD
(B) COX8A MTS
(SEQ ID NO: 299)
MSVLTPLLLRGLTGSARRLPVPRAKIHSL
(C) SOD2 3′UTR
(SEQ ID NO: 300)
ACCACGATCGTTATGCTGATCATACCCTAATGATCCCAGCAAGATAATGTCCTGTCT
TCTAAGATGTGCATCAAGCCTGGTACATACTGAAAACCCTATAAGGTCCTGGATAA
TTTTTGTTTGATTATTCATTGAAGAAACATTTATTTTCCAATTGTGTGAAGTTTTTGA
CTGTTAATAAAAGAATCTGTCAACCATCAAAAAAAAAAAAAAA
(D) ATP5B 3′UTR
(SEQ ID NO: 301)
ACCACGATCGTTATGCTGATCATACCCTAATGATCCCAGCAAGATAATGTCCTGTCT
TCTAAGATGTGCATCAAGCCTGGTACATACTGAAAACCCTATAAGGTCCTGGATAA
TTTTTGTTTGATTATTCATTGAAGAAACATTTATTTTCCAATTGTGTGAAGTTTTTGA
CTGTTAATAAAAGAATCTGTCAACCATCAAAAAAAAAAAAAAA
(E) ND6-DdCBE: Left mitoTALE-G1397-DddAₜₒₓ-N-1x-UGI
(SEQ ID NO: 302)
ATGGCCCTGTCCCGTGCGGTTTGTGGCACCTCCCGTCAACTGGCTCCGGTTCTGGGT
TATCTGGGTTCCCGTCAAAAACACTCCCTGCCGGACTACCCGTATGATGTTCCGGAT
TACGCTGGCTACCCATACGACGTCCCAGACTACGCTGGCTACCCATACGACGTCCC
AGACTACGCTATGGACATCGCGGATCTGCGTACCCTGGGTTACAGCCAGCAGCAGC
AGGAGAAGATCAAGCCGAAGGTGCGCAGCACCGTGGCTCAGCACCACGAAGCCCT
GGTGGGCCACGGTTTCACCCACGCTCACATTGTGGCCCTGAGCCAGCACCCAGCCG
CGCTGGGCACCGTGGCCGTGAAATATCAGGATATGATTGCTGCCCTGCCAGAGGCC
ACCCATGAAGCTATTGTGGGCGTGGGCAAGCAGTGGAGCGGTGCTCGTGCGCTGGA
GGCGCTGCTGACCGTGGCTGGTGAACTGCGTGGTCCGCCGCTGCAGCTGGACACCG
GTCAGCTGCTGAAAATCGCGAAACGTGGCGGTGTGACCGCGGTGGAAGCCGTGCAT
GCTTGGCGTAATGCTCTGACCGGTGCGCCGCTGAACCTGACCCCGCAGCAGGTGGT
GGCTATTGCCAGCAACAACGGCGGTAAACAGGCCCTGGAGACCGTGCAGCGCCTG
CTGCCGGTGCTGTGCCAGGCCCATGGTCTGACCCCGGAGCAGGTGGTGGCGATCGC
TAGCAACATCGGCGGCAAGCAGGCCCTGGAAACCGTGCAGGCGCTGTTACCGGTGC
TGTGCCAGGCTCATGGCCTGACCCCGGAACAAGTIGTGGCTATTGCCAGCCATGAT
GGCGGTAAACAGGCTCTGGAAACCGTGCAGCGTCTGTTGCCGGTGCTGTGCCAAGC
CCATGGCCTGACCCCGGAGCAAGTTGTGGCTATTGCGAGCCATGATGGCGGCAAGC
AGGCGCTGGAAACCGTTCAGCGCCTGTTACCGGTGCTGTGCCAAGCTCATGGTCTG
ACCCCGGAACAGGTGGTGGCCATTGCTTCCCATGATGGCGGTAAACAGGCCCTGGA
AACCGTTCAGCGTCTGCTTCCGGTGCTGTGCCAGGCCCATGGGCTGACCCCGGAAC
AAGTGGTTGCTATTGCCAGCCACGATGGCGGCAAGCAGGCTCTGGAGACCGTTCAG
CGCCTGCTTCCGGTGCTGTGCCAGGCCCATGGCTTAACCCCGGAACAAGTTGTTGCT
ATTGCTAGTCATGATGGCGGTAAACAGGCGCTGGAGACCGTTCAGCGTCTGTTACC
GGTGCTGTGCCAGGCGCATGGCTTAACCCCGGAGCAGGTTGTTGCCATTGCCTCCA
ATATCGGCGGCAAGCAGGCTCTGGAAACCGTTCAGGCCCTGTTGCCGGTGCTGTGC
CAGGCCCATGGACTGACCCCGCAGCAAGTTGTTGCCATTGCCAGCAATGGCGGTGG
CAAACAGGCGCTGGAAACTGTTCAGCGCCTGCTCCCGGTGCTGTGCCAAGCGCATG
GTCTGACCCCGCAGCAAGTGGTTGCTATTGCTAGCAATGGTGGCGGTCGTCCGGCG
CTGGAAAGCATTGTGGCTCAGCTGAGCCGTCCAGACCCGGCCCTGGCGGCTCTGAC
CAACGATCACCTGGTGGCGCTGGCTTGCCTGGGCGGTCGTCCGGCCCTGGATGCGG
TGAAGAAAGGCCTGGGTGGATCCGGCAGCTACGCCCTGGGTCCGTATCAGATTAGC
GCCCCGCAGCTGCCAGCTTACAATGGTCAGACCGTGGGTACCTTCTACTATGTGAA
CGACGCGGGCGGTCTGGAGAGCAAGGTGTTTAGCAGCGGCGGTCCAACCCCGTACC
CAAACTATGCCAATGCCGGTCATGTGGAGGGTCAGAGCGCCCTGTTCATGCGTGAT
AACGGCATCAGCGAGGGTCTGGTGTTCCACAACAACCCGGAAGGCACCTGCGGTTT
TTGCGTGAACATGACCGAGACCCTGCTGCCGGAAAACGCGAAAATGACCGTGGTGC
CGCCGGAAGGTTCTGGCGGCTCAACTAATCTGAGCGACATCATTGAGAAGGAGACT
GGGAAACAGCTGGTCATTCAGGAGTCCATCCTGATGCTGCCTGAGGAGGTGGAGGA
AGTGATCGGCAACAAGCCAGAGTCTGACATCCTGGTGCACACCGCCTACGACGAGT
CCACAGATGAGAATGTGATGCTGCTGACCTCTGACGCCCCCGAGTATAAGCCTTGG
GCCCTGGTCATCCAGGATTCTAACGGCGAGAATAAGATCAAGATGCTGTGATAAAC
CACGATCGTTATGCTGATCATACCCTAATGATCCCAGCAAGATAATGTCCTGTCTTC
TAAGATGTGCATCAAGCCTGGTACATACTGAAAACCCTATAAGGTCCTGGATAATT
TTTGTTTGATTATTCATTGAAGAAACATTTATTTTCCAATTGTGTGAAGTTTTTGACT
GTTAATAAAAGAATCTGTCAACCATCAAA
(F) ND6-DdCBE: Right mitoTALE-G1397-DddAₜₒₓ-N-1x-UGI
(SEQ ID NO: 303)
TCCGTTCTGACCCCGCTGCTGCTGCGTGGCCTGACCGGCTCCGCTCGTCGTCTGCCA
GTTCCGCGTGCGAAAATCCATTCCCTGGACTACAAAGACCATGACGGTGATTATAA
AGATCATGACATCGATTACAAGGATGACGATGACAAGATGGACATCGCGGATCTGC
GTACCCTGGGTTACAGCCAGCAGCAGCAGGAGAAGATCAAGCCAAAGGTGCGCAG
CACCGTGGCCCAGCACCATGAAGCTCTGGTGGGTCACGGCTTCACCCACGCGCACA
TCGTGGCTCTGAGCCAGCACCCAGCCGCGCTGGGTACCGTGGCCGTGAAATATCAG
GACATGATTGCTGCCCTGCCAGAGGCTACCCATGAAGCGATTGTGGGTGTGGGCAA
GAGAGGAGCCGGTGCTCGTGCGCTGGAGGCCCTGCTGACCGTGGCCGGTGAACTGC
GTGGCCCGCCGCTGCAGCTGGATACCGGCCAGCTGCTGAAAATCGCGAAACGTGGC
GGTGTGACCGCTGTGGAAGCTGTGCATGCCTGGCGTAATGCTCTGACCGGTGCCCC
GCTGAACCTGACCCCGCAGCAGGTGGTGGCTATTGCCAGCAACAACGGCGGTAAAC
AGGCTCTGGAAACCGTGCAGCGCCTGCTGCCGGTGCTGTGCCAGGCTCATGGTCTG
ACCCCGGAGCAGGTGGTGGCGATCGCTAGCAACATCGGCGGCAAGCAGGCTCTGG
AGACCGTTCAGGCCCTGTTACCGGTGCTGTGCCAAGCCCATGGTCTGACCCCGCAG
CAAGTTGTGGCTATTGCCAGCAATGGCGGTGGCAAACAGGCGCTGGAGACCGTGCA
GCGTCTGTTGCCGGTGCTGTGCCAAGCCCATGGGCTGACCCCGCAGCAAGTGGTTG
CCATCGCCAGCAACAACGGTGGCAAGCAGGCCCTGGAGACCGTTCAGCGCCTGTTA
CCGGTGCTGTGCCAGGCCCATGGCTTAACCCCGCAGCAAGTTGTGGCCATCGCTAG
CAACAACGGTGGCAAACAGGCTCTGGAGACTGTTCAGCGTCTGCTTCCGGTGCTGT
GCCAAGCGCATGGCCTGACCCCGGAACAAGTTGTTGCTATTGCCAGCCATGATGGT
GGCAAGCAGGCGCTGGAAACCGTTCAGCGCCTGCTTCCGGTGCTGTGCCAGGCGCA
TGGATTAACCCCGCAGCAAGTGGTGGCCATCGCCAGCAATGGTGGCGGTAAACAG
GCCCTGGAAACCGTTCAGCGTCTGTTACCGGTGCTGTGCCAGGCCCATGGATTAAC
CCCGGAACAAGTIGTGGCTATTGCGTCCAATATCGGCGGCAAGCAGGCGCTGGAAA
CTGTGCAGGCTCTGCTCCCGGTGCTGTGCCAGGCCCATGGGTTAACCCCGCAGCAG
GTTGTTGCCATTGCGAGCAACGGCGGTGGCAAACAGGCTCTGGAGACGGTTCAGCG
CCTGCTCCCGGTGCTGTGCCAGGCCCATGGTTTAACCCCGCAGCAGGTGGTTGCTAT
TGCTAGCAATGGCGGCGGCAAGCAGGCGCTGGAAACGGTGCAGCGTCTGCTACCG
GTGCTGTGCCAGGCACATGGCCTTACCCCGCAGCAAGTTGTGGCCATTGCTAGCAA
TGGCGGTGGCCGTCCGGCCCTGGAAAGCATTGTGGCGCAGCTGAGCCGTCCAGACC
CGGCCCTGGCGGCTCTGACCAATGATCACCTGGTGGCCCTGGCCTGCCTGGGTGGC
CGTCCGGCTCTGGATGCCGTGAAGAAAGGTCTGGGCGGATCCGCCATTCCAGTGAA
GCGCGGCGCTACCGGTGAAACCAAAGTGTTTACCGGTAACAGCAACAGCCCGAAG
AGCCCGACCAAAGGCGGTTGCTCTGGCGGCTCAACTAATCTGAGCGACATCATTGA
GAAGGAGACTGGGAAACAGCTGGTCATTCAGGAGTCCATCCTGATGCTGCCTGAGG
AGGTGGAGGAAGTGATCGGCAACAAGCCAGAGTCTGACATCCTGGTGCACACCGC
CTACGACGAGTCCACAGATGAGAATGTGATGCTGCTGACCTCTGACGCCCCCGAGT
ATAAGCCTTGGGCCCTGGTCATCCAGGATTCTAACGGCGAGAATAAGATCAAGATG
CTGTGATAAGGGGTCTTTGTCCTCTGTACTGTCTCTCTCCTTGCCCCTAACCCAAAA
AGCTTCATTTTTCTGTGTAGGCTGCACAAGAGCCTTGATTGAAGATATATTCTTTCT
GAACAGTATTTAAGGTTTCCAATAAAATGTACACCCCTCAGAA
(G) ND1-DdCBE Right mitoTALE repeat
(SEQ ID NO: 304)
GACATCGCGGATCTGCGTACCCTGGGTTACAGCCAGCAGCAGCAGGAGAAGATCA
AGCCAAAGGTGCGCAGCACCGTGGCCCAGCACCATGAAGCTCTGGTGGGTCACGG
CTTCACCCACGCGCACATCGTGGCTCTGAGCCAGCACCCAGCCGCGCTGGGTACCG
TGGCCGTGAAATATCAGGACATGATTGCTGCCCTGCCAGAGGCTACCCATGAAGCG
ATTGTGGGTGTGGGCAAGCAGTGGAGCGGTGCTCGTGCGCTGGAGGCCCTGCTGAC
CGTGGCCGGTGAACTGCGTGGCCCGCCGCTGCAGCTGGATACCGGCCAGCTGCTGA
AAATCGCGAAACGTGGCGGTGTGACCGCTGTGGAAGCTGTGCATGCCTGGCGTAAT
GCTCTGACCGGTGCCCCGCTGAACCTGACCCCGGAACAGGTGGTTGCCATCGCATC
CAATAATGGTGGTAAACAAGCTCTGGAGACCGTTCAAGCCCTGCTGCCAGTGCTGT
GCCAGGCTCATGGTCTGACCCCGCAGCAAGTTGTGGCTATTGCCAGCAACATCGGC
GGCAAGCAGGCCCTGGAGACCGTGCAGCGTCTGCTGCCGGTGCTGTGCCAGGCCCA
TGGCCTGACCCCGCAGCAAGTGGTTGCTATCGCCAGCAACAACGGCGGTAAACAGG
CTCTGGAAACCGTGCAGCGCCTGTTACCGGTGCTGTGCCAAGCCCATGGTCTGACC
CCGGAGCAGGTGGTGGCGATTGCTAGCAACGGCGGTGGCAAGCAGGCTCTGGAGA
CCGTTCAGGCCCTGCTTCCGGTGCTGTGCCAAGCGCATGGCCTGACCCCGGAACAA
GTTGTTGCCATTGCCAGCAATGGTGGCGGTAAACAGGCGCTGGAAACCGTGCAGGC
TCTGTTACCGGTGCTGTGCCAGGCCCATGGGCTGACCCCGGAGCAAGTGGTGGCTA
TTGCGAGCAATGGCGGTGGCAAGCAGGCCCTGGAAACCGTGCAGGCGCTGTTGCCG
GTGCTGTGCCAAGCCCATGGATTAACCCCGGAACAAGTGGTGGCGATCGCTAGCAA
CAACGGTGGCAAACAGGCGCTGGAGACCGTTCAGCGTCTGTTACCGGTGCTGTGCC
AGGCGCATGGCTTAACCCCGGAACAGGTTGTTGCGATTGCCAGCAACATTGGTGGC
AAGCAGGCTCTGGAAACCGTTCAGGCCCTGCTCCCGGTGCTGTGCCAGGCCCATGG
TTTAACCCCGGAACAGGTGGTGGCCATTGCCAGCAACGGTGGCGGTAAACAGGCCC
TGGAGACCGTTCAGCGCCTGCTACCGGTGCTGTGCCAGGCCCATGGACTGACCCCG
GAGCAAGTTGTTGCCATTGCTAGCAACAACGGCGGCAAGCAGGCGCTGGAGACCG
TGCAGGCTCTGCTTCCGGTGCTGTGCCAGGCCCATGGGTTAACCCCGGAGCAGGTT
GTGGCCATCGCCAGCCACGACGGCGGTAAACAGGCCCTGGAAACCGTTCAGGCGCT
GCTACCGGTGCTGTGCCAGGCACATGGCTTAACCCCGGAGCAGGTGGTTGCCATCG
CCTCCAATGGCGGTGGCAAGCAGGCTCTGGAAACGGTGCAGGCCCTGCTGCCGGTG
CTGTGCCAAGCCCATGGGTTGACCCCGGAACAAGTGGTGGCTATTGCTAGCCACGA
CGGTGGCAAACAGGCTCTGGAGACTGTTCAGCGTCTGCTTCCGGTGCTGTGCCAGG
CTCATGGCTTAACCCCGCAGCAAGTTGTTGCTATTGCCTCCAATATTGGTGGCAAGC
AGGCGCTGGAAACCGTTCAGCGCCTGCTGCCGGTGCTGTGCCAGGCTCATGGGCTT
ACCCCGGAACAAGTTGTGGCCATTGCCTCCCATGATGGTGGCAAACAGGCGCTGGA
AACTGTGCAGGCTCTGCTCCCGGTGCTGTGCCAGGCTCATGGATTAACCCCGCAGC
AAGTGGTGGCCATTGCTAGCCACGATGGTGGCAAGCAGGCCCTGGAGACGGTTCAG
CGTCTGCTCCCGGTGCTGTGCCAGGCCCATGGGCTAACCCCGCAGCAGGTTGTTGCT
ATTGCCAGTCATGATGGTGGCAAACAGGCTCTGGAAACTGTGCAGCGCCTGCTACC
GGTGCTGTGCCAGGCTCACGGTCTGACCCCGCAGCAGGTGGTGGCAATCGCAAGCA
ACGGTGGTGGTCGTCCGGCACTGGAAAGCATTGTGGCGCAGCTGAGCCGTCCAGAC
CCGGCCCTGGCGGCTCTGACCAATGATCACCTGGTGGCCCTGGCCTGCCTGGGTGG
CCGTCCGGCTCTGGATGCCGTGAAGAAAGGTCTGGGC

Translated amino acid sequence:

(SEQ ID NO: 305)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNNGGKQALETVQALLPVLCQAHGL
TPQQVVAIASNIGGKQALETVQRLLPVLCQAHGLTPQQVVAIASNNGGKQALETVQRL
LPVLCQAHGLTPEQVVAIASNGGGKQALETVQALLPVLCQAHGLTPEQVVAIASNGGG
KQALETVQALLPVLCQAHGLTPEQVVAIASNGGGKQALETVQALLPVLCQAHGLTPEQ
VVAIASNNGGKQALETVQRLLPVLCQAHGLTPEQVVAIASNIGGKQALETVQALLPVL
CQAHGLTPEQVVAIASNGGGKQALETVQRLLPVLCQAHGLTPEQVVAIASNNGGKQA
LETVQALLPVLCQAHGLTPEQVVAIASHDGGKQALETVQALLPVLCQAHGLTPEQVVA
IASNGGGKQALETVQALLPVLCQAHGLTPEQVVAIASHDGGKQALETVQRLLPVLCQA
HGLTPQQVVAIASNIGGKQALETVQRLLPVLCQAHGLTPEQVVAIASHDGGKQALETV
QALLPVLCQAHGLTPQQVVAIASHDGGKQALETVQRLLPVLCQAHGLTPQQVVAIASH
DGGKQALETVQRLLPVLCQAHGLTPQQVVAIASNGGGRPALESIVAQLSRPDPALAAL
TNDHLVALACLGGRPALDAVKKGLG
(H) ND1-DdCBE Left mitoTALE repeat
(SEQ ID NO: 306)
GACATCGCGGATCTGCGTACCCTGGGTTACAGCCAGCAGCAGCAGGAGAAGATCA
AGCCGAAGGTGCGCAGCACCGTGGCTCAGCACCACGAAGCCCTGGTGGGCCACGG
TTTCACCCACGCTCACATTGTGGCCCTGAGCCAGCACCCAGCCGCGCTGGGCACCG
TGGCCGTGAAATATCAGGATATGATTGCTGCCCTGCCAGAGGCCACCCATGAAGCT
ATTGTGGGCGTGGGCAAGCAGTGGAGCGGTGCTCGTGCGCTGGAGGCGCTGCTGAC
CGTGGCTGGTGAACTGCGTGGTCCGCCGCTGCAGCTGGACACCGGTCAGCTGCTGA
AAATCGCGAAACGTGGCGGTGTGACCGCGGTGGAAGCCGTGCATGCTTGGCGTAAT
GCTCTGACCGGTGCGCCGCTGAACCTGACCCCGGAACAAGTGGTTGCTATCGCATC
CCATGACGGCGGTAAACAAGCCCTGGAGACCGTTCAAGCCCTGCTGCCAGTGCTGT
GCCAGGCTCATGGTCTGACCCCGCAGCAGGTGGTGGCTATTGCCAGCAATGGCGGT
GGCAAGCAGGCGCTGGAGACCGTGCAGCGTCTGCTGCCGGTGCTGTGCCAAGCCCA
TGGCCTGACCCCGCAGCAAGTTGTGGCTATCGCCAGCAACATTGGTGGCAAACAGG
CCCTGGAAACCGTGCAGCGCCTGTTACCGGTGCTGTGCCAGGCCCATGGTCTGACC
CCGGAGCAGGTGGTGGCGATCGCTAGCAACAACGGTGGCAAGCAGGCTCTGGAAA
CCGTGCAGGCCCTGCTTCCGGTGCTGTGCCAGGCCCATGGGCTGACCCCGGAACAA
GTTGTGGCTATTGCCAGCCACGACGGTGGCAAACAGGCGCTGGAAACCGTGCAGGC
TCTGTTACCGGTGCTGTGCCAAGCGCATGGCCTGACCCCGGAACAGGTGGTGGCTA
TTGCTAGCCACGATGGTGGCAAGCAGGCCCTGGAGACCGTTCAGGCGCTGTTGCCG
GTGCTGTGCCAGGCGCATGGCTTAACCCCGGAACAAGTTGTTGCGATTGCTAGCAA
CGGTGGCGGTAAACAGGCTCTGGAGACCGTTCAGCGTCTGTTACCGGTGCTGTGCC
AGGCACATGGCCTGACCCCGGAGCAAGTTGTTGCCATTGCCAGCAACATCGGCGGC
AAGCAGGCTCTGGAGACCGTGCAGGCCCTGCTCCCGGTGCTGTGCCAGGCCCATGG
CTTAACCCCGGAGCAAGTIGTGGCCATTGCCAGCAACAACGGCGGTAAACAGGCGC
TGGAGACCGTTCAGCGCCTGCTACCGGTGCTGTGCCAGGCTCATGGCTTGACCCCG
GAACAGGTTGTTGCGATTGCGAGCCATGATGGCGGCAAGCAGGCGCTGGAAACCG
TTCAGGCTCTGCTTCCGGTGCTGTGCCAGGCCCATGGATTAACCCCGGAGCAGGTT
GTTGCTATTGCCAGCCATGATGGCGGTAAACAGGCCCTGGAGACCGTGCAGGCGCT
GCTACCGGTGCTGTGCCAGGCTCATGGGCTGACCCCGGAGCAAGTGGTTGCTATCG
CGAGCAACAATGGCGGCAAGCAGGCTCTGGAAACGGTGCAGGCCCTGCTGCCGGT
GCTGTGCCAGGCCCATGGGTTAACCCCGGAACAAGTGGTGGCCATCGCTAGCAACG
GCGGTGGCAAACAGGCCCTGGAGACTGTTCAGCGTCTGCTTCCGGTGCTGTGCCAG
GCCCATGGGCTAACCCCGCAGCAAGTGGTTGCCATTGCCAGCAATGGCGGCGGCAA
GCAGGCTCTGGAAACTGTGCAGCGCCTGCTGCCGGTGCTGTGCCAGGCTCACGGTC
TGACCCCGCAACAGGTGGTGGCAATCGCAAGCAATGGTGGTGGTCGTCCGGCACTG
GAGAGCATTGTGGCTCAGCTGAGCCGTCCAGACCCGGCCCTGGCGGCTCTGACCAA
CGATCACCTGGTGGCGCTGGCTTGCCTGGGCGGTCGTCCGGCCCTGGATGCGGTGA
AGAAAGGCCTGGGT

Translated amino acid sequence:

(SEQ ID NO: 307)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNGGGKQALETVQRLLPVLCQAHGLTPQQVVAIASNIGGKQALETVQRL
LPVLCQAHGLTPEQVVAIASNNGGKQALETVQALLPVLCQAHGLTPEQVVAIASHDGG
KQALETVQALLPVLCQAHGLTPEQVVAIASHDGGKQALETVQALLPVLCQAHGLTPEQ
VVAIASNGGGKQALETVQRLLPVLCQAHGLTPEQVVAIASNIGGKQALETVQALLPVL
CQAHGLTPEQVVAIASNNGGKQALETVQRLLPVLCQAHGLTPEQVVAIASHDGGKQA
LETVQALLPVLCQAHGLTPEQVVAIASHDGGKQALETVQALLPVLCQAHGLTPEQVVA
IASNNGGKQALETVQALLPVLCQAHGLTPEQVVAIASNGGGKQALETVQRLLPVLCQA
HGLTPQQVVAIASNGGGKQALETVQRLLPVLCQAHGLTPQQVVAIASNGGGRPALESI
VAQLSRPDPALAALTNDHLVALACLGGRPALDAVKKGLG
(I) ND2-DdCBE Right mitoTALE repeat
(SEQ ID NO: 308)
GACATCGCGGATCTGCGTACCCTGGGTTACAGCCAGCAGCAGCAGGAGAAGATCA
AGCCAAAGGTGCGCAGCACCGTGGCCCAGCACCATGAAGCTCTGGTGGGTCACGG
CTTCACCCACGCGCACATCGTGGCTCTGAGCCAGCACCCAGCCGCGCTGGGTACCG
TGGCCGTGAAATATCAGGACATGATTGCTGCCCTGCCAGAGGCTACCCATGAAGCG
ATTGTGGGTGTGGGCAAGCAGTGGAGCGGTGCTCGTGCGCTGGAGGCCCTGCTGAC
CGTGGCCGGTGAACTGCGTGGCCCGCCGCTGCAGCTGGATACCGGCCAGCTGCTGA
AAATCGCGAAACGTGGCGGTGTGACCGCTGTGGAAGCTGTGCATGCCTGGCGTAAT
GCTCTGACCGGTGCCCCGCTGAACCTGACCCCGGAACAAGTGGTTGCCATCGCATC
CAATATCGGTGGTAAACAAGCCCTGGAGACCGTTCAAGCCCTGCTGCCAGTGCTGT
GCCAGGCTCATGGTCTGACCCCGCAGCAGGTGGTGGCCATTGCGAGCAACAATGGC
GGCAAGCAGGCGCTGGAGACCGTGCAGCGTCTGCTGCCGGTGCTGTGCCAAGCCCA
TGGCCTGACCCCGCAGCAAGTGGTTGCTATCGCCAGCAACATTGGCGGTAAACAGG
CCCTGGAAACCGTGCAGCGCCTGTTACCGGTGCTGTGCCAGGCCCATGGTCTGACC
CCGGAGCAGGTGGTGGCGATCGCTAGCAACATCGGCGGCAAGCAGGCTCTGGAAA
CCGTGCAGGCCCTGCTTCCGGTGCTGTGCCAGGCCCATGGGCTGACCCCGGAACAA
GTTGTGGCTATTGCCAGCCATGATGGCGGTAAACAGGCGCTGGAAACCGTGCAGGC
TCTGTTACCGGTGCTGTGCCAAGCGCATGGCCTGACCCCGGAACAGGTGGTGGCTA
TTGCGAGCAATGGCGGTGGCAAGCAGGCCCTGGAGACCGTTCAGGCGCTGTTGCCG
GTGCTGTGCCAGGCGCATGGCTTAACCCCGGAACAAGTTGTTGCGATCGCTAGCAA
CAACGGTGGCAAACAGGCTCTGGAGACCGTTCAGCGTCTGTTACCGGTGCTGTGCC
AGGCACATGGCCTGACCCCGGAGCAAGTTGTTGCCATTGCCAGCCACGATGGTGGC
AAGCAGGCTCTGGAGACCGTGCAGGCCCTGCTCCCGGTGCTGTGCCAGGCCCATGG
CTTAACCCCGGAGCAAGTTGTGGCTATCGCCAGCAACGGTGGCGGTAAACAGGCGC
TGGAGACCGTTCAGCGCCTGCTACCGGTGCTGTGCCAGGCTCATGGCTTGACCCCG
GAACAGGTTGTTGCCATTGCGTCCAATATCGGCGGCAAGCAGGCGCTGGAAACCGT
TCAGGCTCTGCTTCCGGTGCTGTGCCAGGCCCATGGATTAACCCCGGAGCAGGTTG
TGGCGATTGCGAGCAACGGCGGTGGCAAACAGGCCCTGGAGACCGTGCAGGCGCT
GCTACCGGTGCTGTGCCAGGCTCATGGGCTGACCCCGGAGCAAGTGGTTGCTATTG
CTAGCAATGGCGGCGGCAAGCAGGCTCTGGAAACGGTGCAGGCCCTGCTGCCGGT
GCTGTGCCAGGCCCATGGGTTAACCCCGGAACAAGTGGTGGCCATCGCTTCCAATA
TTGGCGGTAAACAGGCCCTGGAGACTGTTCAGCGTCTGCTTCCGGTGCTGTGCCAG
GCCCATGGGCTAACCCCGCAGCAAGTTGTTGCTATTGCCTCCAATGGCGGTGGCAA
GCAGGCTCTGGAAACTGTGCAGCGCCTGCTGCCGGTGCTGTGCCAGGCTCACGGCC
TGACCCCGCAGCAAGTTGTGGCAATCGCAAGCAATGGTGGTGGTCGTCCGGCTCTG
GAGAGCATTGTGGCGCAGCTGAGCCGTCCAGACCCGGCCCTGGCGGCTCTGACCAA
TGATCACCTGGTGGCCCTGGCCTGCCTGGGTGGCCGTCCGGCTCTGGATGCCGTGA
AGAAAGGTCTGGGC

Translated amino acid sequence:

(SEQ ID NO: 309)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNIGGKQALETVQALLPVLCQAHGL
TPQQVVAIASNNGGKQALETVQRLLPVLCQAHGLTPQQVVAIASNIGGKQALETVQRL
LPVLCQAHGLTPEQVVAIASNIGGKQALETVQALLPVLCQAHGLTPEQVVAIASHDGG
KQALETVQALLPVLCQAHGLTPEQVVAIASNGGGKQALETVQALLPVLCQAHGLTPEQ
VVAIASNNGGKQALETVQRLLPVLCQAHGLTPEQVVAIASHDGGKQALETVQALLPVL
CQAHGLTPEQVVAIASNGGGKQALETVQRLLPVLCQAHGLTPEQVVAIASNIGGKQAL
ETVQALLPVLCQAHGLTPEQVVAIASNGGGKQALETVQALLPVLCQAHGLTPEQVVAI
ASNGGGKQALETVQALLPVLCQAHGLTPEQVVAIASNIGGKQALETVQRLLPVLCQAH
GLTPQQVVAIASNGGGKQALETVQRLLPVLCQAHGLTPQQVVAIASNGGGRPALESIV
AQLSRPDPALAALTNDHLVALACLGGRPALDAVKKGLG
(J) ND2-DdCBE Left mitoTALE repeat
(SEQ ID NO: 310)
GACATCGCGGATCTGCGTACCCTGGGTTACAGCCAGCAGCAGCAGGAGAAGATCA
AGCCAAAGGTGCGCAGCACCGTGGCCCAGCACCATGAAGCTCTGGTGGGTCACGG
CTTCACCCACGCGCACATCGTGGCTCTGAGCCAGCACCCAGCCGCGCTGGGTACCG
TGGCCGTGAAATATCAGGACATGATTGCTGCCCTGCCAGAGGCTACCCATGAAGCG
ATTGTGGGTGTGGGCAAGCAGTGGAGCGGTGCTCGTGCGCTGGAGGCCCTGCTGAC
CGTGGCCGGTGAACTGCGTGGCCCGCCGCTGCAGCTGGATACCGGCCAGCTGCTGA
AAATCGCGAAACGTGGCGGTGTGACCGCTGTGGAAGCTGTGCATGCCTGGCGTAAT
GCTCTGACCGGTGCCCCGCTGAACCTGACCCCGGAACAAGTGGTGGCTATCGCGTC
CCATGATGGTGGTAAACAGGCTCTGGAGACCGTGCAAGCTCTGCTGCCAGTGCTGT
GCCAGGCCCATGGTCTGACCCCGCAGCAGGTGGTGGCTATTGCCAGCAATGGCGGT
GGCAAGCAGGCGCTGGAGACCGTGCAGCGTCTGCTGCCGGTGCTGTGCCAGGCTCA
TGGCCTGACCCCGCAGCAAGTIGTGGCTATTGCCAGCAACGGTGGCGGTAAACAGG
CCCTGGAGACCGTGCAGCGCCTGTTACCGGTGCTGTGCCAAGCCCATGGCCTGACC
CCGGAGCAGGTGGTGGCGATCGCTAGCAACATCGGCGGCAAGCAGGCTCTGGAAA
CCGTGCAGGCCCTGCTTCCGGTGCTGTGCCAGGCCCATGGCTTAACCCCGGAACAG
GTTGTTGCTATTGCCAGCAACAACGGCGGTAAACAGGCGCTGGAAACCGTGCAGGC
TCTGTTACCGGTGCTGTGCCAGGCCCATGGGCTGACCCCGGAACAAGTTGTGGCTA
TTGCGAGCCATGATGGCGGCAAGCAGGCCCTGGAAACCGTGCAGGCGCTGTTGCCG
GTGCTGTGCCAAGCCCATGGATTAACCCCGGAACAAGTTGTTGCGATCGCTAGCAA
CATTGGCGGTAAACAGGCTCTGGAAACCGTTCAGCGTCTGTTACCGGTGCTGTGCC
AGGCGCATGGTCTGACCCCGGAACAGGTTGTGGCCATTGCCTCCAATGGCGGTGGC
AAGCAGGCTCTGGAGACCGTTCAGGCCCTGCTCCCGGTGCTGTGCCAAGCGCATGG
CCTGACCCCGGAACAGGTGGTGGCTATCGCCAGCAACATTGGTGGCAAACAGGCGC
TGGAGACCGTTCAGCGCCTGCTACCGGTGCTGTGCCAGGCACATGGTCTGACCCCG
GAGCAAGTTGTGGCCATTGCTAGCCACGATGGTGGCAAGCAGGCGCTGGAAACCGT
TCAGGCTCTGCTTCCGGTGCTGTGCCAGGCCCATGGTTTAACCCCGGAACAAGTGG
TTGCCATTGCGTCCAATGGTGGCGGTAAACAGGCCCTGGAAACCGTTCAGGCGCTG
CTACCGGTGCTGTGCCAGGCTCATGGGCTGACCCCGGAGCAAGTGGTTGCTATTGC
TTCCCATGATGGCGGCAAGCAGGCTCTGGAAACGGTGCAGGCCCTGCTGCCGGTGC
TGTGCCAAGCCCATGGGTTAACCCCGGAACAGGTGGTTGCGATTGCTAGCCACGAC
GGCGGTAAACAGGCCCTGGAAACGGTTCAGCGTCTGCTTCCGGTGCTGTGCCAGGC
CCATGGACTTACCCCGCAGCAGGTTGTGGCGATTGCCTCCAATGGCGGTGGCAAGC
AGGCTCTGGAAACTGTGCAGCGCCTGCTGCCGGTGCTGTGCCAGGCTCATGGTTTA
ACCCCGGAGCAGGTTGTTGCCATCGCCAGCCACGACGGTGGCAAACAGGCGCTGG
AAACTGTGCAGGCTCTGCTCCCGGTGCTGTGCCAGGCTCATGGACTTACCCCGGAG
CAGGTGGTTGCCATTGCTAGCAACATTGGTGGCAAGCAGGCCCTGGAGACTGTTCA
GGCGCTGTTACCGGTGCTGTGCCAGGCCCATGGGTTAACCCCGGAGCAAGTTGTTG
CCATTGCCTCCAATATTGGTGGCAAACAGGCTCTGGAGACTGTTCAGGCCCTGCTG
CCGGTGCTGTGCCAGGCTCACGGTCTGACCCCGCAGCAAGTGGTGGCAATCGCAAG
CAATGGTGGTGGTCGTCCGGCTCTGGAGAGCATTGTGGCGCAGCTGAGCCGTCCAG
ACCCGGCCCTGGCGGCTCTGACCAATGATCACCTGGTGGCCCTGGCCTGCCTGGGT
GGCCGTCCGGCTCTGGATGCCGTGAAGAAAGGTCTGGGC

Translated amino acid sequence:

(SEQ ID NO: 311)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNIGGKQALETVQALLPVLCQAHGL
TPQQVVAIASNNGGKQALETVQRLLPVLCQAHGLTPQQVVAIASNIGGKQALETVQRL
LPVLCQAHGLTPEQVVAIASNIGGKQALETVQALLPVLCQAHGLTPEQVVAIASHDGG
KQALETVQALLPVLCQAHGLTPEQVVAIASNGGGKQALETVQALLPVLCQAHGLTPEQ
VVAIASNNGGKQALETVQRLLPVLCQAHGLTPEQVVAIASHDGGKQALETVQALLPVL
CQAHGLTPEQVVAIASNGGGKQALETVQRLLPVLCQAHGLTPEQVVAIASNIGGKQAL
ETVQALLPVLCQAHGLTPEQVVAIASNGGGKQALETVQALLPVLCQAHGLTPEQVVAI
ASNGGGKQALETVQALLPVLCQAHGLTPEQVVAIASNIGGKQALETVQRLLPVLCQAH
GLTPQQVVAIASNGGGKQALETVQRLLPVLCQAHGLTPQQVVAIASNGGGRPALESIV
AQLSRPDPALAALTNDHLVALACLGGRPALDAVKKGLG
(K) ND4-DdCBE Right mitoTALE repeat
(SEQ ID NO: 312)
GACATCGCGGATCTGCGTACCCTGGGTTACAGCCAGCAGCAGCAGGAGAAGATCA
AGCCAAAGGTGCGCAGCACCGTGGCCCAGCACCATGAAGCTCTGGTGGGTCACGG
CTTCACCCACGCGCACATCGTGGCTCTGAGCCAGCACCCAGCCGCGCTGGGTACCG
TGGCCGTGAAATATCAGGACATGATTGCTGCCCTGCCAGAGGCTACCCATGAAGCG
ATTGTGGGTGTGGGCAAGCAGTGGAGCGGTGCTCGTGCGCTGGAGGCCCTGCTGAC
CGTGGCCGGTGAACTGCGTGGCCCGCCGCTGCAGCTGGATACCGGCCAGCTGCTGA
AAATCGCGAAACGTGGCGGTGTGACCGCTGTGGAAGCTGTGCATGCCTGGCGTAAT
GCTCTGACCGGTGCCCCGCTGAACCTGACCCCGGAACAAGTGGTTGCGATTGCGTC
CCATGATGGTGGTAAACAAGCCCTGGAGACCGTTCAAGCTCTGCTGCCAGTGCTGT
GCCAGGCTCATGGTCTGACCCCGCAGCAGGTGGTGGCTATTGCCAGCCATGATGGC
GGCAAGCAGGCTCTGGAGACCGTGCAGCGTCTGCTGCCGGTGCTGTGCCAAGCCCA
TGGCCTGACCCCGCAGCAAGTTGTGGCTATTGCCAGCAACGGCGGTGGCAAACAGG
CGCTGGAAACCGTGCAGCGCCTGTTACCGGTGCTGTGCCAAGCGCATGGTCTGACC
CCGGAGCAGGTGGTGGCCATCGCTAGCAACAACGGTGGCAAGCAGGCCCTGGAAA
CCGTGCAGGCGCTGTTGCCGGTGCTGTGCCAGGCCCATGGCTTAACCCCGGAACAA
GTGGTGGCCATTGCGAGCAATGGTGGCGGTAAACAGGCTCTGGAAACCGTGCAGG
CCCTGCTTCCGGTGCTGTGCCAGGCCCATGGGCTGACCCCGGAACAAGTTGTGGCT
ATCGCCAGCAACATCGGCGGCAAGCAGGCGCTGGAGACCGTTCAGGCTCTGTTACC
GGTGCTGTGCCAGGCGCATGGCCTGACCCCGGAACAGGTGGTGGCGATCGCTAGCA
ACATTGGCGGTAAACAGGCCCTGGAGACCGTTCAGCGCCTGCTCCCGGTGCTGTGC
CAGGCCCATGGTCTGACCCCGGAACAGGTTGTTGCTATTGCCAGCAACAACGGCGG
CAAGCAGGCCCTGGAGACCGTGCAGGCGCTGCTACCGGTGCTGTGCCAGGCCCATG
GACTGACCCCGGAGCAGGTTGTGGCCATCGCGTCCAATGGCGGTGGCAAACAGGCT
CTGGAGACCGTTCAGCGTCTGTTACCGGTGCTGTGCCAGGCACATGGCCTGACCCC
GGAGCAAGTTGTTGCCATCGCTAGCAACATTGGTGGCAAGCAGGCGCTGGAAACCG
TTCAGCGCCTGCTACCGGTGCTGTGCCAGGCTCATGGCTTAACCCCGGAGCAGGTT
GTCGCCATTGCCAGCAACAATGGTGGCAAACAGGCTCTGGAAACTGTGCAGGCCCT
GCTACCGGTGCTGTGCCAGGCCCATGGGTTAACCCCGGAACAGGTTGTGGCCATTG
CCTCCAATAACGGTGGCAAGCAGGCGCTGGAAACGGTGCAGGCTCTGCTTCCGGTG
CTGTGCCAGGCTCATGGGCTGACCCCGGAGCAAGTGGTTGCTATTGCGTCCAACAT
TGGTGGCAAACAGGCCCTGGAAACCGTTCAGGCGCTGCTCCCGGTGCTGTGCCAGG
CCCATGGGCTAACCCCGGAACAGGTGGTTGCCATTGCCTCCAACAATGGTGGCAAG
CAGGCCCTGGAAACGGTTCAGCGTCTGCTTCCGGTGCTGTGCCAGGCCCATGGGCT
TACCCCGCAGCAAGTTGTTGCTATCGCCAGCAATATTGGTGGCAAACAGGCTCTGG
AAACGGTGCAGCGCCTGCTACCGGTGCTGTGCCAGGCTCATGGTTTAACCCCGCAG
CAGGTGGTTGCGATTGCCTCCAACAACGGTGGCAAGCAGGCGCTGGAAACTGTTCA
GCGTCTGCTCCCGGTGCTGTGCCAGGCTCACGGCCTGACCCCGCAGCAAGTGGTGG
CTATCGCCTCCAACGGTGGTGGTCGCCCGGCTCTGGAAAGCATTGTGGCGCAGCTG
AGCCGTCCAGACCCGGCCCTGGCGGCTCTGACCAATGATCACCTGGTGGCCCTGGC
CTGCCTGGGTGGCCGTCCGGCTCTGGATGCCGTGAAGAAAGGTCTGGGC

Translated amino acid sequence:

(SEQ ID NO: 313)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASHDGGKQALETVQRLLPVLCQAHGLTPQQVVAIASNGGGKQALETVQR
LLPVLCQAHGLTPEQVVAIASNNGGKQALETVQALLPVLCQAHGLTPEQVVAIASNGG
GKQALETVQALLPVLCQAHGLTPEQVVAIASNIGGKQALETVQALLPVLCQAHGLTPE
QVVAIASNIGGKQALETVQRLLPVLCQAHGLTPEQVVAIASNNGGKQALETVQALLPV
LCQAHGLTPEQVVAIASNGGGKQALETVQRLLPVLCQAHGLTPEQVVAIASNIGGKQA
LETVQRLLPVLCQAHGLTPEQVVAIASNNGGKQALETVQALLPVLCQAHGLTPEQVVA
IASNNGGKQALETVQALLPVLCQAHGLTPEQVVAIASNIGGKQALETVQALLPVLCQA
HGLTPEQVVAIASNNGGKQALETVQRLLPVLCQAHGLTPQQVVAIASNIGGKQALETV
QRLLPVLCQAHGLTPQQVVAIASNNGGKQALETVQRLLPVLCQAHGLTPQQVVAIASN
GGGRPALESIVAQLSRPDPALAALTNDHLVALACLGGRPALDAVKKGLG
(L) ND4-DdCBE Left mitoTALE repeat
(SEQ ID NO: 314)
GACATCGCGGATCTGCGTACCCTGGGTTACAGCCAGCAGCAGCAGGAGAAGATCA
AGCCAAAGGTGCGCAGCACCGTGGCCCAGCACCATGAAGCTCTGGTGGGTCACGG
CTTCACCCACGCGCACATCGTGGCTCTGAGCCAGCACCCAGCCGCGCTGGGTACCG
TGGCCGTGAAATATCAGGACATGATTGCTGCCCTGCCAGAGGCTACCCATGAAGCG
ATTGTGGGTGTGGGCAAGCAGTGGAGCGGTGCTCGTGCGCTGGAGGCCCTGCTGAC
CGTGGCCGGTGAACTGCGTGGCCCGCCGCTGCAGCTGGATACCGGCCAGCTGCTGA
AAATCGCGAAACGTGGCGGTGTGACCGCTGTGGAAGCTGTGCATGCCTGGCGTAAT
GCTCTGACCGGTGCCCCGCTGAACCTGACCCCGGAACAGGTGGTGGCAATCGCAAG
CAATAATGGTGGTAAACAGGCTCTGGAAACCGTGCAAGCTCTGCTGCCAGTTCTGT
GCCAGGCTCATGGTCTGACCCCGCAGCAGGTGGTGGCTATTGCCAGCCATGATGGC
GGCAAGCAGGCCCTGGAGACCGTGCAGCGTCTGCTGCCGGTGCTGTGCCAGGCCCA
TGGCCTGACCCCGCAGCAAGTIGTGGCTATTGCCAGCAACGGCGGTGGCAAACAGG
CTCTGGAGACCGTGCAGCGCCTGTTACCGGTGCTGTGCCAAGCCCATGGTCTGACC
CCGGAGCAGGTGGTGGCGATCGCTAGCAACATTGGTGGCAAGCAGGCCCTGGAAA
CCGTGCAGGCGCTGTTGCCGGTGCTGTGCCAAGCCCATGGGCTGACCCCGGAACAA
GTTGTTGCCATTGCCAGCAACAATGGTGGCAAACAGGCTCTGGAAACTGTGCAGGC
CCTGCTTCCGGTGCTGTGCCAGGCCCATGGATTAACCCCGGAACAAGTTGTGGCTA
TTGCGAGCAATGGCGGCGGCAAGCAGGCGCTGGAAACCGTGCAGGCTCTGTTACCG
GTGCTGTGCCAGGCGCATGGCCTGACCCCGGAGCAAGTGGTGGCCATCGCTAGCAA
CATTGGCGGTAAACAGGCGCTGGAGACCGTTCAGCGTCTGTTACCGGTGCTGTGCC
AGGCACATGGCCTTACCCCGGAACAAGTTGTGGCCATTGCCAGCAACATCGGCGGC
AAGCAGGCCCTGGAAACGGTGCAGGCGCTGCTCCCGGTGCTGTGCCAGGCCCATGG
GTTAACCCCGGAACAAGTGGTTGCTATTGCTAGCCATGATGGCGGTAAACAGGCCC
TGGAGACCGTTCAGCGCCTGCTACCGGTGCTGTGCCAGGCCCATGGTTTAACCCCG
GAACAGGTTGTTGCGATTGCTAGCCACGATGGCGGCAAGCAGGCTCTGGAGACCGT
TCAGGCCCTGCTCCCGGTGCTGTGCCAGGCCCATGGGCTTACCCCGGAGCAAGTTG
TTGCTATTGCCTCCAATATTGGCGGTAAACAGGCGCTGGAAACCGTTCAGGCTCTG
CTTCCGGTGCTGTGCCAGGCTCATGGCCTCACCCCGGAACAAGTTGTGGCGATTGC
GTCCCATGATGGCGGCAAGCAGGCCCTGGAAACTGTGCAGGCGCTGCTACCGGTGC
TGTGCCAGGCCCATGGGCTAACCCCGGAACAGGTGGTTGCGATTGCTAGCAACAAC
GGCGGTAAACAGGCTCTGGAGACTGTTCAGCGTCTGCTTCCGGTGCTGTGCCAGGC
TCATGGGCTGACCCCGCAGCAAGTGGTTGCTATTGCCAGCAATGGCGGTGGCAAGC
AGGCGCTGGAGACTGTTCAGCGCCTGCTCCCGGTGCTGTGCCAGGCTCATGGTTTA
ACCCCGGAGCAGGTTGTGGCGATCGCCAGCAATGGTGGCGGTAAACAGGCTCTGG
AAACGGTGCAGGCCCTGCTCCCGGTGCTGTGCCAGGCTCATGGACTGACCCCGGAG
CAAGTTGTTGCCATTGCGTCCCACGACGGCGGCAAGCAGGCGCTGGAGACGGTGCA
GGCTCTGCTCCCGGTGCTGTGCCAGGCTCACGGTCTGACCCCGCAACAGGTGGTGG
CAATCGCAAGCAACGGTGGTGGTCGTCCGGCACTGGAGAGCATTGTGGCGCAGCTG
AGCCGTCCAGACCCGGCCCTGGCGGCTCTGACCAATGATCACCTGGTGGCCCTGGC
CTGCCTGGGTGGCCGTCCGGCTCTGGATGCCGTGAAGAAAGGTCTGGGC

Translated amino acid sequence:

(SEQ ID NO: 315)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNNGGKQALETVQALLPVLCQAHGL
TPQQVVAIASHDGGKQALETVQRLLPVLCQAHGLTPQQVVAIASNGGGKQALETVQR
LLPVLCQAHGLTPEQVVAIASNIGGKQALETVQALLPVLCQAHGLTPEQVVAIASNNG
GKQALETVQALLPVLCQAHGLTPEQVVAIASNGGGKQALETVQALLPVLCQAHGLTPE
QVVAIASNIGGKQALETVQRLLPVLCQAHGLTPEQVVAIASNIGGKQALETVQALLPVL
CQAHGLTPEQVVAIASHDGGKQALETVQRLLPVLCQAHGLTPEQVVAIASHDGGKQA
LETVQALLPVLCQAHGLTPEQVVAIASNIGGKQALETVQALLPVLCQAHGLTPEQVVAI
ASHDGGKQALETVQALLPVLCQAHGLTPEQVVAIASNNGGKQALETVQRLLPVLCQA
HGLTPQQVVAIASNGGGKQALETVQRLLPVLCQAHGLTPEQVVAIASNGGGKQALETV
QALLPVLCQAHGLTPEQVVAIASHDGGKQALETVQALLPVLCQAHGLTPQQVVAIASN
GGGRPALESIVAQLSRPDPALAALTNDHLVALACLGGRPALDAVKKGLG
(M) ND5.1-DdCBE Right mitoTALE repeat
(SEQ ID NO: 316)
GACATCGCGGATCTGCGTACCCTGGGTTACAGCCAGCAGCAGCAGGAGAAGATCA
AGCCAAAGGTGCGCAGCACCGTGGCCCAGCACCATGAAGCTCTGGTGGGTCACGG
CTTCACCCACGCGCACATCGTGGCTCTGAGCCAGCACCCAGCCGCGCTGGGTACCG
TGGCCGTGAAATATCAGGACATGATTGCTGCCCTGCCAGAGGCTACCCATGAAGCG
ATTGTGGGTGTGGGCAAGCAGTGGAGCGGTGCTCGTGCGCTGGAGGCCCTGCTGAC
CGTGGCCGGTGAACTGCGTGGCCCGCCGCTGCAGCTGGATACCGGCCAGCTGCTGA
AAATCGCGAAACGTGGCGGTGTGACCGCTGTGGAAGCTGTGCATGCCTGGCGTAAT
GCTCTGACCGGTGCCCCGCTGAACCTGACCCCACAGCAGGTGGTGGCAATCGCAAG
CCACGACGGAGGCAAGCAGGCCCTGGAGACCGTGCAGAGGCTGCTGCCCGTGCTG
TGCCAGGCACACGGACTGACACCTGAACAGGTCGTGGCAATCGCATCCAACGGAG
GCGGCAAGCAGGCCCTGGAAACCGTGCAGCGCCTGTTACCCGTGCTGTGCCAGGCC
CACGGCCTGACACCCCAGCAGGTGGTGGCCATCGCCTCTAATGGAGGGGGCAAGC
AGGCCCTGGAGACGGTGCAGCGGCTGCTGCCTGTGCTGTGCCAGGCTCATGGACTG
ACACCAGAACAGGTGGTCGCAATCGCAAGCAACGGAGGTGGCAAGCAGGCCCTGG
AGACTGTGCAGGCCCTGCTTCCCGTGCTGTGCCAGGCTCACGGACTGACACCTCAG
CAGGTCGTCGCCATCGCCTCCAACAATGGTGGCAAGCAGGCCCTGGAGACAGTGCA
GAGACTGCTGCCAGTGCTGTGCCAAGCCCATGGACTGACACCACAGCAGGTCGTCG
CTATCGCCTCTAATAACGGCGGCAAGCAGGCCCTGGAGACGGTACAGAGGCTGTTA
CCCGTGCTGTGCCAAGCACACGGACTGACACCAGAGCAGGTCGTCGCAATCGCCAG
CAATATCGGTGGCAAGCAGGCCCTGGAGACGGTCCAGCGCCTGCTCCCCGTGCTGT
GCCAAGCCCACGGCCTGACCCCTCAGCAGGTCGTGGCTATTGCTAGCAATAACGGG
GGCAAGCAGGCCCTGGAGACGGTTCAGCGGCTGTTGCCCGTGCTGTGCCAAGCCCA
CGGTCTGACCCCTCAGCAGGTGGTCGCTATTGCTTCTAATGGAGGAGGCAAGCAGG
CCCTGGAGACGGTACAGAGACTGTTACCTGTGCTGTGCCAGGCACATGGCCTGACA
CCAGAGCAGGTGGTCGCTATCGCCAGCAACATAGGTGGCAAGCAGGCCCTGGAGA
CGGTACAGAGGCTGCTTCCCGTGCTGTGCCAAGCTCATGGCCTGACACCTGAACAG
GTGGTCGCCATTGCTAGCAATAACGGTGGCAAGCAGGCCCTGGAGACGGTACAGC
GGCTGTTACCAGTGCTGTGCCAAGCACATGGCTTAACCCCTCAACAGGTCGTCGCA
ATTGCCTCTAATATCGGAGGCAAGCAGGCCCTGGAGACGGTACAGCGGCTGCTCCC
CGTGCTGTGCCAGGCGCACGGCCTGACTCCTCAGCAGGTCGTGGCAATCGCCAGCA
ACATCGGCGGCAGACCTGCCCTGGAGAGCATTGTGGCGCAGCTGAGCCGTCCAGAC
CCGGCCCTGGCGGCTCTGACCAATGATCACCTGGTGGCCCTGGCCTGCCTGGGTGG
CCGTCCGGCTCTGGATGCCGTGAAGAAAGGTCTGGGC

Translated amino acid sequence:

(SEQ ID NO: 317)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASHDGGKQALETVQRLLPVLCQAHGL
TPEQVVAIASNGGGKQALETVQRLLPVLCQAHGLTPQQVVAIASNGGGKQALETVQRL
LPVLCQAHGLTPEQVVAIASNGGGKQALETVQALLPVLCQAHGLTPQQVVAIASNNGG
KQALETVQRLLPVLCQAHGLTPQQVVAIASNNGGKQALETVQRLLPVLCQAHGLTPEQ
VVAIASNIGGKQALETVQRLLPVLCQAHGLTPQQVVAIASNNGGKQALETVQRLLPVL
CQAHGLTPQQVVAIASNGGGKQALETVQRLLPVLCQAHGLTPEQVVAIASNIGGKQAL
ETVQRLLPVLCQAHGLTPEQVVAIASNNGGKQALETVQRLLPVLCQAHGLTPQQVVAI
ASNIGGKQALETVQRLLPVLCQAHGLTPQQVVAIASNIGGRPALESIVAQLSRPDPALA
ALTNDHLVALACLGGRPALDAVKKGLG
(N) ND5.1-DdCBE Left mitoTALE repeat
(SEQ ID NO: 318)
CTGACCCCTGAGCAGGTGGTGGCCATCGCCAGCAATATCGGAGGCAAGCAGGCCCT
GGAGACCGTGCAGGCCCTGCTGCCCGTGCTGTGCCAGGCACACGGACTGACACCTC
AGCAGGTCGTCGCCATCGCCTCCAACAATGGCGGCAAGCAGGCCCTGGAAACCGTG
CAGAGGCTGTTACCCGTGCTGTGCCAGGCCCACGGCCTGACACCCCAGCAGGTGGT
GGCAATCGCATCTCACGATGGGGGCAAGCAGGCCCTGGAGACGGTGCAGCGCCTG
CTGCCTGTGCTGTGCCAGGCTCATGGACTGACACCAGAACAGGTCGTGGCCATCGC
CAGCAACATTGGCGGCAAGCAGGCCCTGGAGACTGTCCAGGCCCTGTTACCCGTGC
TGTGCCAAGCCCATGGACTGACACCTGAACAGGTCGTGGCAATCGCATCCAATGGA
GGTGGCAAGCAGGCCCTGGAGACAGTGCAGGCCCTGCTGCCAGTGCTGTGCCAGGC
TCACGGCCTGACACCAGAACAGGTGGTCGCAATCGCATCTAATGGAGGAGGCAAG
CAGGCCCTGGAGACGGTACAGGCCCTGTTGCCCGTGCTGTGCCAAGCCCACGGACT
GACACCAGAGCAGGTCGTCGCTATTGCTTCCAACATTGGAGGCAAGCAGGCCCTGG
AGACGGTCCAGCGGCTGCTTCCCGTGCTGTGCCAAGCTCATGGCCTGACACCAGAG
CAGGTGGTCGCTATTGCCTCCAACAATGGAGGCAAGCAGGCCCTGGAGACGGTTCA
GGCCCTGCTTCCCGTGCTGTGCCAGGCTCATGGTCTGACACCCGAACAGGTGGTCG
CTATCGCCTCTCACGATGGAGGCAAGCAGGCCCTGGAGACGGTACAGAGGCTGTTA
CCTGTGCTGTGCCAGGCCCATGGGCTGACCCCAGAACAGGTGGTCGCCATCGCCAG
CAACATCGGCGGCAAGCAGGCCCTGGAGACGGTACAGGCCCTGCTCCCCGTGCTGT
GCCAAGCACATGGCCTGACACCCGAGCAGGTCGTGGCTATTGCTAGCAACAACGGG
GGCAAGCAGGCCCTGGAGACGGTACAGGCCCTGCTACCAGTGCTGTGCCAAGCGC
ACGGGCTGACCCCAGAGCAGGTCGTCGCAATCGCCTCTAACAACGGTGGCAAGCA
GGCCCTGGAGACGGTACAGGCCCTGCTGCCCGTGCTGTGCCAAGCGCATGGGCTGA
CTCCAGAACAGGTGGTGGCTATCGCCAGCAACATTGGAGGCAAGCAGGCCCTGGA
GACGGTACAGCGGCTGCTACCCGTGCTGTGCCAAGCGCACGGTCTGACACCTCAGC
AGGTGGTCGCTATCGCTTCTAACATAGGGGGCAAGCAGGCCCTGGAGACGGTACAG
CGGCTGCTGCCCGTGCTGTGCCAAGCGCACGGACTGACCCCACAGCAGGTCGTCGC
TATCGCCTCTAACGGAGGAGGCAGACCCGCCCTGGAG

Translated amino acid sequence:

(SEQ ID NO: 319)
LTPEQVVAIASNIGGKQALETVQALLPVLCQAHGLTPQQVVAIASNNGGKQALETVQR
LLPVLCQAHGLTPQQVVAIASHDGGKQALETVQRLLPVLCQAHGLTPEQVVAIASNIG
GKQALETVQALLPVLCQAHGLTPEQVVAIASNGGGKQALETVQALLPVLCQAHGLTPE
QVVAIASNGGGKQALETVQALLPVLCQAHGLTPEQVVAIASNIGGKQALETVQRLLPV
LCQAHGLTPEQVVAIASNNGGKQALETVQALLPVLCQAHGLTPEQVVAIASHDGGKQ
ALETVQRLLPVLCQAHGLTPEQVVAIASNIGGKQALETVQALLPVLCQAHGLTPEQVV
AIASNNGGKQALETVQALLPVLCQAHGLTPEQVVAIASNNGGKQALETVQALLPVLCQ
AHGLTPEQVVAIASNIGGKQALETVQRLLPVLCQAHGLTPQQVVAIASNIGGKQALETV
QRLLPVLCQAHGLTPQQVVAIASNGGGRPALE
(O) ND5.2-DdCBE Right mitoTALE repeat
(SEQ ID NO: 320)
GACATCGCGGATCTGCGTACCCTGGGTTACAGCCAGCAGCAGCAGGAGAAGATCA
AGCCGAAGGTGCGCAGCACCGTGGCTCAGCACCACGAAGCCCTGGTGGGCCACGG
TTTCACCCACGCTCACATCGTGGCCCTGAGCCAGCACCCAGCCGCGCTGGGCACCG
TGGCCGTGAAATATCAGGACATGATTGCTGCCCTGCCAGAGGCCACCCATGAAGCT
ATTGTGGGCGTGGGCAAGCAGTGGAGCGGTGCTCGTGCGCTGGAGGCGCTGCTGAC
CGTGGCTGGTGAACTGCGTGGTCCGCCGCTGCAGCTGGATACCGGTCAGCTGCTGA
AAATTGCCAAACGTGGCGGTGTGACCGCGGTGGAAGCCGTGCATGCTTGGCGTAAT
GCTCTGACCGGTGCGCCGCTGAACCTGACCCCGCAGCAGGTGGTGGCTATTGCCAG
CAACAACGGCGGTAAACAGGCTCTGGAGACCGTGCAGCGTCTGCTGCCGGTGCTGT
GCCAGGCTCATGGTCTGACCCCGGAGCAGGTGGTGGCCATTGCTAGCCATGATGGC
GGCAAGCAGGCGCTGGAAACCGTGCAGCGCCTGTTACCGGTGCTGTGCCAAGCCCA
TGGTCTGACCCCGCAGCAAGTIGTGGCTATTGCGAGCAACGGCGGTGGCAAACAGG
CCCTGGAAACCGTTCAGCGTCTGTTACCGGTGCTGTGCCAGGCCCATGGCCTGACC
CCGGAACAAGTGGTGGCTATCGCCAGCAACATTGGTGGCAAGCAGGCCCTGGAAA
CCGTGCAGGCGCTGTTGCCGGTGCTGTGCCAAGCCCATGGGCTGACCCCGCAGCAA
GTGGTTGCGATCGCTAGCAACAACGGTGGCAAACAGGCTCTGGAAACCGTTCAGCG
CCTGCTTCCGGTGCTGTGCCAAGCGCATGGCTTAACCCCGCAGCAAGTTGTGGCCA
TTGCGAGCAACAACGGTGGCAAGCAGGCGCTGGAGACCGTTCAGCGTCTGCTTCCG
GTGCTGTGCCAGGCGCATGGCCTGACCCCGGAGCAAGTGGTGGCTATTGCTAGCCA
CGATGGTGGCAAACAGGCCCTGGAGACCGTGCAGCGCCTGCTCCCGGTGCTGTGCC
AGGCCCATGGATTAACCCCGCAGCAAGTGGTGGCCATCGCCAGCAATGGCGGCGG
CAAGCAGGCTCTGGAAACTGTGCAGCGTCTGTTACCGGTGCTGTGCCAGGCCCATG
GGTTAACCCCGCAGCAGGTTGTTGCCATTGCCTCCAATAATGGCGGTAAACAGGCG
CTGGAGACTGTGCAGCGCCTGCTACCGGTGCTGTGCCAGGCACATGGTCTGACCCC
GGAACAAGTTGTTGCCATTGCGTCCCATGATGGCGGCAAGCAGGCCCTGGAGACTG
TTCAGCGTCTGCTCCCGGTGCTGTGCCAGGCCCATGGTTTAACCCCGGAACAAGTTG
TGGCCATTGCTAGCCACGATGGCGGTAAACAGGCTCTGGAAACTGTTCAGCGCCTG
CTGCCGGTGCTGTGCCAAGCACATGGCTTAACCCCGGAACAGGTTGTTGCTATTGC
CAGCAACATCGGCGGCAAGCAGGCTCTGGAGACCGTTCAGGCCCTGTTGCCGGTGC
TGTGCCAGGCCCATGGGCTTACCCCGGAACAAGTGGTTGCCATCGCCAGCAACATT
GGCGGTAAACAGGCGCTGGAAACCGTTCAGGCTCTGTTGCCGGTGCTGTGCCAGGC
TCATGGCCTTACCCCGCAGCAAGTTGTGGCGATTGCTAGCAATGGCGGTGGCAAGC
AGGCGCTGGAGACGGTTCAGCGTCTGCTACCGGTGCTGTGCCAGGCTCATGGATTG
ACCCCGCAGCAGGTCGTGGCCATTGCCTCCAATAACGGTGGCAAACAGGCGCTGGA
GACAGTTCAGCGCCTGCTGCCGGTGCTGTGCCAGGCTCATGGGTTGACCCCGCAGC
AGGTAGTTGCTATTGCTAGCAATGGTGGCGGTCGTCCGGCCCTGGAGAGCATTGTG
GCGCAGCTGAGCCGTCCAGACCCGGCGCTGGCGGCTCTGACCAACGATCACCTGGT
GGCGCTGGCTTGCCTGGGCGGTCGTCCGGCCCTGGATGCCGTGAAGAAAGGCCTGG
GT

Translated amino acid sequence:

(SEQ ID NO: 321)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASNNGGKQALETVQRLLPVLCQAHGL
TPEQVVAIASHDGGKQALETVQRLLPVLCQAHGLTPQQVVAIASNGGGKQALETVQRL
LPVLCQAHGLTPEQVVAIASNIGGKQALETVQALLPVLCQAHGLTPQQVVAIASNNGG
KQALETVQRLLPVLCQAHGLTPQQVVAIASNNGGKQALETVQRLLPVLCQAHGLTPEQ
VVAIASHDGGKQALETVQRLLPVLCQAHGLTPQQVVAIASNGGGKQALETVQRLLPVL
CQAHGLTPQQVVAIASNNGGKQALETVQRLLPVLCQAHGLTPEQVVAIASHDGGKQA
LETVQRLLPVLCQAHGLTPEQVVAIASHDGGKQALETVQRLLPVLCQAHGLTPEQVVA
IASNIGGKQALETVQALLPVLCQAHGLTPEQVVAIASNIGGKQALETVQALLPVLCQAH
GLTPQQVVAIASNGGGKQALETVQRLLPVLCQAHGLTPQQVVAIASNNGGKQALETV
QRLLPVLCQAHGLTPQQVVAIASNGGGRPALESIVAQLSRPDPALAALTNDHLVALAC
LGGRPALDAVKKGLG
(P) ND5.2-DdCBE Left mitoTALE repeat
(SEQ ID NO: 322)
GACATCGCGGATCTGCGTACCCTGGGTTACAGCCAGCAGCAGCAGGAGAAGATCA
AGCCGAAGGTGCGCAGCACCGTGGCTCAGCACCACGAAGCCCTGGTGGGCCACGG
TTTCACCCACGCTCACATTGTGGCCCTGAGCCAGCACCCAGCCGCGCTGGGCACCG
TGGCCGTGAAATATCAGGATATGATTGCTGCCCTGCCAGAGGCCACCCATGAAGCT
ATTGTGGGCGTGGGCAAGCAGTGGAGCGGTGCTCGTGCGCTGGAGGCGCTGCTGAC
CGTGGCTGGTGAACTGCGTGGTCCGCCGCTGCAGCTGGACACCGGTCAGCTGCTGA
AAATCGCGAAACGTGGCGGTGTGACCGCGGTGGAAGCCGTGCATGCTTGGCGTAAT
GCTCTGACCGGTGCGCCGCTGAACCTGACCCCTGAGCAGGTGGTGGCAATCGCAAG
CCACGACGGAGGCAAGCAGGCCCTGGAGACAGTGCAGGCCCTGCTGCCCGTGCTGT
GCCAGGCACACGGCCTGACACCTGAGCAGGTGGTGGCCATCGCCTCCAACATCGGC
GGCAAGCAGGCCCTGGAGACAGTACAGAGGCTGTTACCCGTGCTGTGCCAGGCCCA
CGGCCTGACACCCCAGCAGGTCGTCGCCATCGCCTCTAATATTGGAGGCAAGCAGG
CCCTGGAGACAGTCCAGCGCCTGCTGCCTGTGCTGTGCCAGGCTCATGGCCTGACA
CCAGAACAGGTCGTGGCCATCGCCAGTAATATTGGGGGCAAGCAGGCCCTGGAGA
CAGTTCAGGCCCTGTTACCCGTGCTGTGCCAAGCCCATGGCCTGACACCTGAACAG
GTGGTCGCCATCGCCTCCAATATTGGTGGCAAGCAGGCCCTGGAGACAGTACAGGC
CCTGCTGCCAGTGCTGTGCCAGGCTCACGGCCTGACACCAGAGCAGGTCGTCGCAA
TCGCATCTCATGATGGCGGCAAGCAGGCCCTGGAGACAGTACAGGCCCTGTTACCC
GTGCTGTGCCAAGCGCACGGCCTGACCCCTGAACAGGTCGTGGCTATTGCAAGCCA
CGATGGTGGCAAGCAGGCCCTGGAGACAGTACAGCGGCTGCTTCCCGTGCTGTGCC
AAGCTCATGGCCTGACACCTGAGCAGGTCGTCGCTATTGCTAGCAATATTGGCGGC
AAGCAGGCCCTGGAGACAGTACAGGCCCTGCTCCCCGTGCTGTGCCAAGCACACGG
CCTGACACCCGAACAGGTGGTGGCTATCGCCTCTAATGGAGGTGGCAAGCAGGCCC
TGGAGACAGTACAGAGGCTGCTTCCTGTGCTGTGCCAGGCCCATGGCCTGACCCCT
GAGCAGGTCGTGGCTATTGCCAGTAATATAGGAGGCAAGCAGGCCCTGGAGACAG
TACAGGCCCTGCTACCCGTGCTGTGCCAAGCGCATGGCCTGACCCCAGAACAGGTC
GTGGCAATCGCATCTCATGACGGCGGCAAGCAGGCCCTGGAGACAGTACAGGCCCT
GCTACCAGTGCTGTGCCAAGCACATGGCCTGACCCCCGAACAGGTGGTGGCAATCG
CCTCTCACGACGGGGGCAAGCAGGCCCTGGAGACAGTACAGGCCCTGCTACCCGTG
CTGTGCCAAGCGCACGGCCTGACGCCAGAACAGGTGGTCGCTATCGCAAGCAACG
GCGGTGGCAAGCAGGCCCTGGAGACAGTACAGCGGCTGCTACCCGTGCTGTGCCAA
GCGCACGGCCTGACTCCTCAGCAGGTCGTCGCTATCGCATCTCATGATGGTGGCAA
GCAGGCCCTGGAGACAGTACAGCGGCTGCTACCCGTGCTGTGCCAAGCGCACGGCC
TGACACCACAGCAGGTCGTCGCAATTGCATCTAACGGAGGAGGCAGACCCGCCCTG
GAGAGCATTGTGGCTCAGCTGAGCCGTCCAGACCCGGCCCTGGCGGCTCTGACCAA
CGATCACCTGGTGGCGCTGGCTTGCCTGGGCGGTCGTCCGGCCCTGGATGCGGTGA
AGAAAGGCCTGGGT

Translated amino acid sequence:

(SEQ ID NO: 323)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPEQVVAIASNIGGKQALETVQRLLPVLCQAHGLTPQQVVAIASNIGGKQALETVQRLL
PVLCQAHGLTPEQVVAIASNIGGKQALETVQALLPVLCQAHGLTPEQVVAIASNIGGKQ
ALETVQALLPVLCQAHGLTPEQVVAIASHDGGKQALETVQALLPVLCQAHGLTPEQVV
AIASHDGGKQALETVQRLLPVLCQAHGLTPEQVVAIASNIGGKQALETVQALLPVLCQ
AHGLTPEQVVAIASNGGGKQALETVQRLLPVLCQAHGLTPEQVVAIASNIGGKQALET
VQALLPVLCQAHGLTPEQVVAIASHDGGKQALETVQALLPVLCQAHGLTPEQVVAIAS
HDGGKQALETVQALLPVLCQAHGLTPEQVVAIASNGGGKQALETVQRLLPVLCQAHG
LTPQQVVAIASHDGGKQALETVQRLLPVLCQAHGLTPQQVVAIASNGGGRPALESIVA
QLSRPDPALAALTNDHLVALACLGGRPALDAVKKGLG
(Q) ND5.3-DdCBE Right mitoTALE repeat
(SEQ ID NO: 324)
GACATCGCGGATCTGCGTACCCTGGGTTACAGCCAGCAGCAGCAGGAGAAGATCA
AGCCAAAGGTGCGCAGCACCGTGGCCCAGCACCATGAAGCTCTGGTGGGTCACGG
CTTCACCCACGCGCACATCGTGGCTCTGAGCCAGCACCCAGCCGCGCTGGGTACCG
TGGCCGTGAAATATCAGGACATGATTGCTGCCCTGCCAGAGGCTACCCATGAAGCG
ATTGTGGGTGTGGGCAAGCAGTGGAGCGGTGCTCGTGCGCTGGAGGCCCTGCTGAC
CGTGGCCGGTGAACTGCGTGGCCCGCCGCTGCAGCTGGATACCGGCCAGCTGCTGA
AAATCGCGAAACGTGGCGGTGTGACCGCTGTGGAAGCTGTGCATGCCTGGCGTAAT
GCTCTGACCGGTGCCCCGCTGAACCTGACACCACAGCAGGTCGTCGCTATCGCTTC
AAACATTGGGGGGAAACAGGCACTGGAAACCGTCCAGAGACTGCTGCCCGTCCTGT
GCCAGGCCCACGGCCTGACCCCTGAGCAGGTGGTGGCCATCGCCAGCAATATCGGA
GGCAAGCAGGCCCTGGAGACCGTGCAGCGGCTGCTGCCCGTGCTGTGCCAAGCCCA
CGGCTTAACACCTCAGCAGGTCGTGGCTATCGCCTCCAACAATGGCGGCAAGCAGG
CCCTGGAGACGGTGCAGAGACTGCTGCCAGTGCTGTGCCAGGCCCACGGCTTAACA
CCAGAACAGGTCGTGGCCATCGCCTCTAACATTGGCGGCAAGCAGGCCCTGGAGAC
TGTGCAGGCCCTGCTGCCCGTGCTGTGCCAGGCCCACGGCCTTACACCACAGCAGG
TGGTGGCAATCGCCAGCAATGGAGGGGGCAAGCAGGCCCTGGAGACAGTGCAGAG
GCTGCTGCCCGTGCTGTGCCAAGCCCACGGCCTGACACCTCAGCAGGTGGTCGCCA
TCGCCTCCAACGGAGGTGGCAAGCAGGCCCTGGAGACGGTACAGCGCCTGCTGCCC
GTGCTGTGCCAAGCCCACGGCCTAACACCCGAACAGGTCGTCGCCATCGCCTCTAA
CATCGGCGGCAAGCAGGCCCTGGAGACGGTCCAGCGGCTGCTGCCTGTGCTGTGCC
AAGCCCACGGCCTTACCCCTCAGCAGGTCGTGGCAATCGCCAGCAACAATGGTGGC
AAGCAGGCCCTGGAGACGGTTCAGAGACTGCTGCCCGTGCTGTGCCAAGCCCACGG
CCTCACACCTCAGCAGGTGGTGGCCATTGCCTCCAACGGAGGAGGCAAGCAGGCCC
TGGAGACGGTACAGAGGCTGCTGCCAGTGCTGTGCCAGGCCCACGGCCTAACACCA
GAACAGGTGGTCGCTATTGCCTCTAACATTGGTGGCAAGCAGGCCCTGGAGACGGT
ACAGCGCCTGCTGCCCGTGCTGTGCCAAGCCCACGGCCTAACGCCAGAACAGGTCG
TCGCTATCGCCAGCAACGGAGGAGGCAAGCAGGCCCTGGAGACGGTACAGCGGCT
GCTGCCCGTGCTGTGCCAAGCCCACGGCCTAACCCCACAGCAGGTCGTGGCCATTG
CCTCCAATAACGGCGGCAAGCAGGCCCTGGAGACGGTACAGCGGCTGCTGCCCGTG
CTGTGCCAAGCCCACGGCCTAACTCCCCAGCAAGTCGTCGCTATTGCCTCTAATAAC
GGGGGCAAGCAGGCCCTGGAGACGGTACAGAGACTGCTGCCCGTGCTGTGCCAAG
CCCACGGCCTGACACCACAGCAGGTCGTCGCCATCGCAAGCAACGGAGGAGGGAG
GCCCGCACTGGAGAGCATTGTGGCGCAGCTGAGCCGTCCAGACCCGGCCCTGGCGG
CTCTGACCAATGATCACCTGGTGGCCCTGGCCTGCCTGGGTGGCCGTCCGGCTCTGG
ATGCCGTGAAGAAAGGTCTGGGC

Translated amino acid sequence:

(SEQ ID NO: 325)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASNIGGKQALETVQRLLPVLCQAHGL
TPEQVVAIASNIGGKQALETVQRLLPVLCQAHGLTPQQVVAIASNNGGKQALETVQRL
LPVLCQAHGLTPEQVVAIASNIGGKQALETVQALLPVLCQAHGLTPQQVVAIASNGGG
KQALETVQRLLPVLCQAHGLTPQQVVAIASNGGGKQALETVQRLLPVLCQAHGLTPEQ
VVAIASNIGGKQALETVQRLLPVLCQAHGLTPQQVVAIASNNGGKQALETVQRLLPVL
CQAHGLTPQQVVAIASNGGGKQALETVQRLLPVLCQAHGLTPEQVVAIASNIGGKQAL
ETVQRLLPVLCQAHGLTPEQVVAIASNGGGKQALETVQRLLPVLCQAHGLTPQQVVAI
ASNNGGKQALETVQRLLPVLCQAHGLTPQQVVAIASNNGGKQALETVQRLLPVLCQA
HGLTPQQVVAIASNGGGRPALESIVAQLSRPDPALAALTNDHLVALACLGGRPALDAV
KKGLG
(R) ND5.3-DdCBE Left mitoTALE repeat
(SEQ ID NO: 326)
GACATCGCGGATCTGCGTACCCTGGGTTACAGCCAGCAGCAGCAGGAGAAGATCA
AGCCGAAGGTGCGCAGCACCGTGGCTCAGCACCACGAAGCCCTGGTGGGCCACGG
TTTCACCCACGCTCACATTGTGGCCCTGAGCCAGCACCCAGCCGCGCTGGGCACCG
TGGCCGTGAAATATCAGGATATGATTGCTGCCCTGCCAGAGGCCACCCATGAAGCT
ATTGTGGGCGTGGGCAAGCAGTGGAGCGGTGCTCGTGCGCTGGAGGCGCTGCTGAC
CGTGGCTGGTGAACTGCGTGGTCCGCCGCTGCAGCTGGACACCGGTCAGCTGCTGA
AAATCGCGAAACGTGGCGGTGTGACCGCGGTGGAAGCCGTGCATGCTTGGCGTAAT
GCTCTGACCGGTGCGCCGCTGAACCTGACTCCCGAACAGGTGGTCGCTATCGCTTCT
CATGATGGCGGAAAACAGGCTCTGGAAACCGTCCAGGCTCTGCTGCCCGTGCTGTG
CCAGGCCCACGGCCTGACCCCACAGCAGGTCGTCGCAATCGCCAGCAATATCGGAG
GCAAGCAGGCCCTGGAGACCGTGCAGCGGCTGCTGCCCGTGCTGTGCCAAGCCCAC
GGCTTAACACCTCAGCAGGTGGTGGCCATCGCCTCCAACAATGGCGGCAAGCAGGC
CCTGGAGACGGTGCAGAGACTGCTGCCAGTGCTGTGCCAGGCCCACGGCTTAACAC
CAGAACAGGTCGTGGCAATCGCCTCTAACGGAGGGGGCAAGCAGGCCCTGGAGAC
TGTGCAGGCCCTGCTGCCCGTGCTGTGCCAGGCCCACGGCCTTACACCAGAACAGG
TGGTCGCCATTGCCAGCAATGGAGGTGGCAAGCAGGCCCTGGAGACAGTCCAGGC
CCTGCTGCCCGTGCTGTGCCAAGCCCACGGCCTGACACCTGAACAGGTGGTCGCAA
TCGCCTCCCACGATGGGGGCAAGCAGGCCCTGGAGACGGTACAGGCCCTGCTGCCC
GTGCTGTGCCAAGCCCACGGCCTAACACCCGAACAGGTGGTGGCCATTGCCTCTAA
CGGAGGAGGCAAGCAGGCCCTGGAGACGGTCCAGCGGCTGCTGCCTGTGCTGTGCC
AAGCCCACGGCCTTACCCCTGAACAAGTCGTGGCCATCGCCAGCAATGGAGGAGGC
AAGCAGGCCCTGGAGACGGTTCAGGCCCTGCTGCCCGTGCTGTGCCAAGCCCACGG
CCTCACACCTGAACAAGTIGTGGCCATCGCCTCCCACGATGGTGGCAAGCAGGCCC
TGGAGACGGTACAGAGGCTGCTGCCAGTGCTGTGCCAGGCCCACGGCCTAACACCA
GAACAGGTGGTGGCTATCGCCTCTAACATTGGCGGCAAGCAGGCCCTGGAGACGGT
ACAGGCCCTGCTGCCCGTGCTGTGCCAAGCCCACGGCCTAACGCCAGAACAGGTCG
TCGCTATTGCCAGCAACATTGGGGGCAAGCAGGCCCTGGAGACGGTACAGGCCCTG
CTGCCCGTGCTGTGCCAAGCCCACGGCCTAACCCCTGAACAGGTGGTGGCAATCGC
CTCCAACATTGGTGGCAAGCAGGCCCTGGAGACGGTACAGGCCCTGCTGCCCGTGC
TGTGCCAAGCCCACGGCCTAACTCCCGAGCAGGTCGTCGCCATCGCCTCTAATGGC
GGCGGCAAGCAGGCCCTGGAGACGGTACAGAGGCTGCTGCCTGTGCTGTGCCAAG
CCCACGGCCTAACGCCGCAGCAAGTCGTCGCTATTGCCAGCAATATTGGCGGCAAG
CAGGCCCTGGAGACGGTACAGCGCCTGCTGCCCGTGCTGTGCCAAGCCCACGGCCT
GACCCCCCAGCAGGTGGTGGCAATCGCTTCAAACGGAGGAGGGAGACCCGCTCTG
GAAAGCATTGTGGCTCAGCTGAGCCGTCCAGACCCGGCCCTGGCGGCTCTGACCAA
CGATCACCTGGTGGCGCTGGCTTGCCTGGGCGGTCGTCCGGCCCTGGATGCGGTGA
AGAAAGGCCTGGGT

Translated amino acid sequence:

(SEQ ID NO: 327)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNIGGKQALETVQRLLPVLCQAHGLTPQQVVAIASNNGGKQALETVQRL
LPVLCQAHGLTPEQVVAIASNGGGKQALETVQALLPVLCQAHGLTPEQVVAIASNGGG
KQALETVQALLPVLCQAHGLTPEQVVAIASHDGGKQALETVQALLPVLCQAHGLTPEQ
VVAIASNGGGKQALETVQRLLPVLCQAHGLTPEQVVAIASNGGGKQALETVQALLPVL
CQAHGLTPEQVVAIASHDGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIA
SNIGGKQALETVQALLPVLCQAHGLTPEQVVAIASNGGGKQALETVQRLLPVLCQAHG
LTPQQVVAIASNIGGKQALETVQRLLPVLCQAHGLTPQQVVAIASNGGGRPALESIVAQ
LSRPDPALAALTNDHLVALACLGGRPALDAVKKGLG
(S) ATP8-DdCBE Right mitoTALE repeat
(SEQ ID NO: 328)
GACATCGCGGATCTGCGTACCCTGGGTTACAGCCAGCAGCAGCAGGAGAAGATCA
AGCCAAAGGTGCGCAGCACCGTGGCCCAGCACCATGAAGCTCTGGTGGGTCACGG
CTTCACCCACGCGCACATCGTGGCTCTGAGCCAGCACCCAGCCGCGCTGGGTACCG
TGGCCGTGAAATATCAGGACATGATTGCTGCCCTGCCAGAGGCTACCCATGAAGCG
ATTGTGGGTGTGGGCAAGCAGTGGAGCGGTGCTCGTGCGCTGGAGGCCCTGCTGAC
CGTGGCCGGTGAACTGCGTGGCCCGCCGCTGCAGCTGGATACCGGCCAGCTGCTGA
AAATCGCGAAACGTGGCGGTGTGACCGCTGTGGAAGCTGTGCATGCCTGGCGTAAT
GCTCTGACCGGTGCCCCGCTGAACCTGACCCCTCAGCAGGTGGTGGCCATCGCCAG
CAACATCGGCGGCAAGCAGGCCCTGGAGACAGTGCAGAGGCTGCTGCCCGTGCTGT
GCCAGGCACACGGCCTGACACCTGAGCAGGTGGTGGCAATCGCATCCAATGGAGG
AGGCAAGCAGGCCCTGGAGACAGTACAGCGCCTGTTACCCGTGCTGTGCCAGGCCC
ACGGCCTGACACCCCAGCAGGTCGTCGCCATCGCCTCTAACAATGGGGGCAAGCAG
GCCCTGGAGACAGTCCAGCGGCTGCTGCCTGTGCTGTGCCAGGCTCATGGCCTGAC
ACCAGAACAGGTCGTGGCTATTGCCAGCAACAATGGTGGCAAGCAGGCCCTGGAG
ACAGTTCAGGCCCTGCTTCCCGTGCTGTGCCAGGCTCACGGCCTGACACCACAGCA
GGTCGTGGCCATCGCCTCCAACAATGGCGGCAAGCAGGCCCTGGAGACAGTACAG
AGACTGCTGCCAGTGCTGTGCCAAGCCCATGGCCTGACCCCTCAGCAGGTCGTGGC
AATCGCATCTCACGACGGTGGCAAGCAGGCCCTGGAGACAGTACAGAGGCTGTTAC
CCGTGCTGTGCCAAGCACACGGCCTGACACCAGAGCAGGTCGTCGCAATCGCAAGC
AACGGCGGCGGCAAGCAGGCCCTGGAGACAGTACAGCGCCTGCTCCCCGTGCTGTG
CCAAGCCCACGGCCTGACACCTCAGCAGGTGGTCGCCATTGCCAGCAACGGCGGGG
GCAAGCAGGCCCTGGAGACAGTACAGCGGCTGTTGCCCGTGCTGTGCCAAGCCCAC
GGCCTGACGCCCCAGCAGGTGGTCGCCATCGCATCTAACGGCGGTGGCAAGCAGGC
CCTGGAGACAGTACAGCGGCTGCTTCCTGTGCTGTGCCAGGCCCATGGCCTGACCC
CCGAACAGGTCGTGGCTATCGCTAGCAACAATGGCGGCAAGCAGGCCCTGGAGAC
AGTACAGAGACTGTTACCCGTGCTGTGCCAAGCGCATGGCCTGACCCCTGAACAGG
TCGTGGCAATTGCCTCCAATAACGGTGGCAAGCAGGCCCTGGAGACAGTACAGCGG
CTGCTACCAGTGCTGTGCCAAGCACATGGCCTGACCCCCCAGCAGGTCGTGGCTAT
TGCATCTAATGGAGGAGGCAGACCCGCCCTGGAGAGCATTGTGGCGCAGCTGAGCC
GTCCAGACCCGGCCCTGGCGGCTCTGACCAATGATCACCTGGTGGCCCTGGCCTGC
CTGGGTGGCCGTCCGGCTCTGGATGCCGTGAAGAAAGGTCTGGGC

Translated amino acid sequence:

(SEQ ID NO: 329)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASNIGGKQALETVQRLLPVLCQAHGL
TPEQVVAIASNGGGKQALETVQRLLPVLCQAHGLTPQQVVAIASNNGGKQALETVQRL
LPVLCQAHGLTPEQVVAIASNNGGKQALETVQALLPVLCQAHGLTPQQVVAIASNNGG
KQALETVQRLLPVLCQAHGLTPQQVVAIASHDGGKQALETVQRLLPVLCQAHGLTPEQ
VVAIASNGGGKQALETVQRLLPVLCQAHGLTPQQVVAIASNGGGKQALETVQRLLPVL
CQAHGLTPQQVVAIASNGGGKQALETVQRLLPVLCQAHGLTPEQVVAIASNNGGKQA
LETVQRLLPVLCQAHGLTPEQVVAIASNNGGKQALETVQRLLPVLCQAHGLTPQQVVA
IASNGGGRPALESIVAQLSRPDPALAALTNDHLVALACLGGRPALDAVKKGLG
(T) ATP8-DdCBE Left mitoTALE repeat
(SEQ ID NO: 330)
GACATCGCGGATCTGCGTACCCTGGGTTACAGCCAGCAGCAGCAGGAGAAGATCA
AGCCGAAGGTGCGCAGCACCGTGGCTCAGCACCACGAAGCCCTGGTGGGCCACGG
TTTCACCCACGCTCACATCGTGGCCCTGAGCCAGCACCCAGCCGCGCTGGGCACCG
TGGCCGTGAAATATCAGGACATGATTGCTGCCCTGCCAGAGGCCACCCATGAAGCT
ATTGTGGGCGTGGGCAAGCAGTGGAGCGGTGCTCGTGCGCTGGAGGCGCTGCTGAC
CGTGGCTGGTGAACTGCGTGGTCCGCCGCTGCAGCTGGATACCGGTCAGCTGCTGA
AAATCGCGAAACGTGGCGGTGTGACCGCGGTGGAAGCCGTGCATGCTTGGCGTAAT
GCTCTGACCGGTGCGCCGCTGAACCTGACCCCGGAGCAGGTGGTGGCTATCGCCAG
CAACATTGGCGGTAAACAGGCCCTGGAAACCGTGCAGGCGCTGCTGCCGGTGCTGT
GCCAGGCTCATGGTCTGACCCCGCAGCAGGTGGTGGCGATCGCTAGCAACGGCGGT
GGCAAGCAGGCTCTGGAGACCGTGCAGCGTCTGTTACCGGTGCTGTGCCAAGCCCA
TGGCCTGACCCCGCAGCAAGTTGTGGCCATTGCGAGCAATGGTGGCGGTAAACAGG
CGCTGGAAACCGTGCAGCGCCTGTTGCCGGTGCTGTGCCAAGCCCATGGGCTGACC
CCGGAACAAGTTGTTGCTATCGCCAGCAACATCGGCGGCAAGCAGGCTCTGGAAAC
CGTGCAGGCCCTGCTTCCGGTGCTGTGCCAAGCGCATGGTCTGACCCCGGAACAAG
TGGTGGCCATCGCTTCCAATATTGGCGGTAAACAGGCGCTGGAGACCGTGCAGGCT
CTGCTCCCGGTGCTGTGCCAAGCACATGGTCTGACCCCGGAGCAAGTTGTGGCTAT
TGCCTCCAATATCGGCGGCAAGCAGGCCCTGGAGACCGTTCAGGCGCTGTTACCGG
TGCTGTGCCAGGCCCATGGATTAACCCCGGAGCAAGTGGTGGCTATTGCTAGCCAT
GATGGCGGTAAACAGGCCCTGGAGACTGTTCAGCGTCTGCTACCGGTGCTGTGCCA
GGCCCATGGTTTAACCCCGGAACAGGTTGTTGCCATCGCTTCCAACATCGGCGGCA
AGCAGGCTCTGGAAACGGTGCAGGCCCTGTTACCGGTGCTGTGCCAGGCCCATGGG
TTAACCCCGGAACAAGTTGTGGCCATTGCCTCCCATGACGGCGGTAAACAGGCTCT
GGAGACCGTTCAGCGCCTGCTACCGGTGCTGTGCCAGGCGCATGGCTTAACCCCGG
AACAAGTGGTTGCCATTGCGTCCAATATCGGCGGCAAGCAGGCGCTGGAGACCGTT
CAGGCTCTGCTTCCGGTGCTGTGCCAGGCACATGGCCTTACCCCGGAACAAGTGGT
CGCGATCGCTTCCAACATTGGCGGTAAACAGGCCCTGGAAACGGTTCAGGCGCTGC
TTCCGGTGCTGTGCCAGGCCCATGGGCTTACCCCGGAACAGGTTGTGGCTATTGCC
AGTAATATCGGCGGCAAGCAGGCTCTGGAAACTGTGCAGGCCCTGCTACCGGTGCT
GTGCCAGGCTCATGGGCTGACCCCGGAGCAAGTGGTTGCCATTGCCTCCCATGATG
GCGGTAAACAGGCGCTGGAAACGGTGCAGCGTCTGCTTCCGGTGCTGTGCCAGGCT
CATGGCTTAACCCCGCAGCAAGTTGTTGCGATTGCTAGCAATGGCGGTGGCAAGCA
GGCCCTGGAAACTGTTCAGCGCCTGTTACCGGTGCTGTGCCAGGCCCATGGGCTAA
CCCCGGAACAGGTGGTTGCTATTGCCAGCAACATTGGTGGCAAACAGGCGCTGGAA
ACTGTGCAGGCTCTGCTTCCGGTGCTGTGCCAGGCCCATGGGCTGACCCCGCAGCA
AGTGGTTGCTATTGCTAGCAATGGTGGCGGTCGTCCGGCCCTGGAGAGCATTGTGG
CGCAGCTGAGCCGTCCAGACCCGGCCCTGGCGGCTCTGACCAACGATCACCTGGTG
GCGCTGGCTTGCCTGGGCGGTCGTCCGGCCCTGGATGCGGTGAAGAAAGGCCTGGG
T

Translated amino acid sequence:

(SEQ ID NO: 331)
DIADLRTLGYSQQQQEKIKPKVRSTVAQHHEALVGHGFTHAHIVALSQHP
AALGTVAVKYQDMIAALPEATHEAIVGVGKQWSGARALEALLTVAGELRG
PPLQLDTGQLLKIAKRGGVTAVEAVHAWRNALTGAPLNLTPEQVVAIASN
IGGKQALETVQALLPVLCQAHGLTPQQVVAIASNGGGKQALETVQRLLPV
LCQAHGLTPQQVVAIASNGGGKQALETVQRLLPVLCQAHGLTPEQVVAIA
SNIGGKQALETVQALLPVLCQAHGLTPEQVVAIASNIGGKQALETVQALL
PVLCQAHGLTPEQVVAIASNIGGKQALETVQALLPVLCQAHGLTPEQVVA
IASHDGGKQALETVQRLLPVLCQAHGLTPEQVVAIASNIGGKQALETVQA
LLPVLCQAHGLTPEQVVAIASHDGGKQALETVQRLLPVLCQAHGLTPEQV
VAIASNIGGKQALETVQALLPVLCQAHGLTPEQVVAIASNIGGKQALETV
QALLPVLCQAHGLTPEQVVAIASNIGGKQALETVQALLPVLCQAHGLTPE
QVVAIASHDGGKQALETVQRLLPVLCQAHGLTPQQVVAIASNGGGKQALE
TVQRLLPVLCQAHGLTPEQVVAIASNIGGKQALETVQALLPVLCQAHGLT
PQQVVAIASNGGGRPALESIVAQLSRPDPALAALTNDHLVALACLGGRPA
LDAVKKGLG

Other exemplary mtDNA base editors may comprise DdCBE/mitoTALE fusion proteins, as follows:

All right-side halves of DdCBEs have the general architecture of (from N- to C-terminus): COX8A MTS-3×FLAG-mitoTALE-2aa linker-DddAtox half-4aa linker-1×-UGI-ATP5B 3′UTR

All left-side halves of DdCBEs have the general architecture of (from N- to C-terminus): SOD2 MTS-3×HA-mitoTALE-2aa linker-DddAtox half-4aa linker-1×-UGI-SOD2 3′UTR

mitoTALE domains are annotated as: bold for N-terminal domain, underlined for RVD and bolded italics for C-terminal domain.

ND6-DdCBE: Left mitoTALE-G1397-DddAtox-N-1x-UGI (Note: Terminal NG
RVD recognizes a mismatched T instead of a G in the reference genome)
(SEQ ID NO: 360)
MALSRAVCGTSRQLAPVLGYLGSRQKHSLPDYPYDVPDYAGYPYDVPDYAGYPYDVP
DYAMDIADLRTLGYSQQQQEKIKPKVRSTVAQHHEALVGHGFTHAHIVALSQHPAAL
GTVAVKYQDMIAALPEATHEAIVGVGKQWSGARALEALLTVAGELRGPPLQLDTGQL
LKIAKRGGVTAVEAVHAWRNALTGAPLNLTPQQVVAIASNNGGKQALETVQRLLPVL
CQAHGLTPEQVVAIASNIGGKQALETVQALLPVLCQAHGLTPEQVVAIASHDGGKQAL
ETVQRLLPVLCQAHGLTPEQVVAIASHDGGKQALETVQRLLPVLCQAHGLTPEQVVAI
ASHDGGKQALETVQRLLPVLCQAHGLTPEQVVAIASHDGGKQALETVQRLLPVLCQA
HGLTPEQVVAIASHDGGKQALETVQRLLPVLCQAHGLTPEQVVAIASNIGGKQALETV
QALLPVLCQAHGLTPQQVVAIASNGGGKQALETVQRLLPVLCQAHGLTPQQVVAIASN
GGGRPALESIVAQLSRPDPALAALTNDHLVALACLGGRPALDAVKKGLGGSGSYALGP
YQISAPQLPAYNGQTVGTFYYVNDAGGLESKVFSSGGPTPYPNYANAGHVEGQSALFM
RDNGISEGLVFHNNPEGTCGFCVNMTETLLPENAKMTVVPPEGSGGSTNLSDIIEKETG
KQLVIQESILMLPEEVEEVIGNKPESDILVHTAYDESTDENVMLLTSDAPEYKPWALVIQ
DSNGENKIKML**
ND6-DdCBE: Right mitoTALE-G1397-DddAtox-N-1x-UGI (Note: Terminal NG
RVD recognizes a mismatched T instead of a G in the reference genome. The NTD was also
engineered to be permissive for A, T, C and G nucleotides at the N0 position)
(SEQ ID NO: 361)
MASVLTPLLLRGLTGSARRLPVPRAKIHSLDYKDHDGDYKDHDIDYKDDDDKMDIAD
LRTLGYSQQQQEKIKPKVRSTVAQHHEALVGHGFTHAHIVALSQHPAALGTVAVKYQ
DMIAALPEATHEAIVGVGKRGAGARALEALLTVAGELRGPPLQLDTGQLLKIAKRGGV
TAVEAVHAWRNALTGAPLNLTPQQVVAIASNNGGKQALETVQRLLPVLCQAHGLTPE
QVVAIASNIGGKQALETVQALLPVLCQAHGLTPQQVVAIASNGGGKQALETVQRLLPV
LCQAHGLTPQQVVAIASNNGGKQALETVQRLLPVLCQAHGLTPQQVVAIASNNGGKQ
ALETVQRLLPVLCQAHGLTPEQVVAIASHDGGKQALETVQRLLPVLCQAHGLTPQQVV
AIASNGGGKQALETVQRLLPVLCQAHGLTPEQVVAIASNIGGKQALETVQALLPVLCQ
AHGLTPQQVVAIASNGGGKQALETVQRLLPVLCQAHGLTPQQVVAIASNGGGKQALE
TVQRLLPVLCQAHGLTPQQVVAIASNGGGRPALESIVAQLSRPDPALAALTNDHLVAL
ACLGGRPALDAVKKGLGGSAIPVKRGATGETKVFTGNSNSPKSPTKGGCSGGSTNLSDI
IEKETGKQLVIQESILMLPEEVEEVIGNKPESDILVHTAYDESTDENVMLLTSDAPEYKP
WALVIQDSNGENKIKML**
ND1-DdCBE Right mitoTALE repeat
(SEQ ID NO: 362)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNNGGKQALETVQALLPVLCQAHGL
TPQQVVAIASNIGGKQALETVQRLLPVLCQAHGLTPQQVVAIASNNGGKQALETVQRL
LPVLCQAHGLTPEQVVAIASNGGGKQALETVQALLPVLCQAHGLTPEQVVAIASNGGG
KQALETVQALLPVLCQAHGLTPEQVVAIASNGGGKQALETVQALLPVLCQAHGLTPEQ
VVAIASNNGGKQALETVQRLLPVLCQAHGLTPEQVVAIASNIGGKQALETVQALLPVL
CQAHGLTPEQVVAIASNGGGKQALETVQRLLPVLCQAHGLTPEQVVAIASNNGGKQA
LETVQALLPVLCQAHGLTPEQVVAIASHDGGKQALETVQALLPVLCQAHGLTPEQVVA
IASNGGGKQALETVQALLPVLCQAHGLTPEQVVAIASHDGGKQALETVQRLLPVLCQA
HGLTPQQVVAIASNIGGKQALETVQRLLPVLCQAHGLTPEQVVAIASHDGGKQALETV
QALLPVLCQAHGLTPQQVVAIASHDGGKQALETVQRLLPVLCQAHGLTPQQVVAIASH
DGGKQALETVQRLLPVLCQAHGLTPQQVVAIASNGGGRPALESIVAQLSRPDPALAAL
TNDHLVALACLGGRPALDAVKKGLG
ND1-DdCBE Left mitoTALE repeat
(SEQ ID NO: 363)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNGGGKQALETVQRLLPVLCQAHGLTPQQVVAIASNIGGKQALETVQRL
LPVLCQAHGLTPEQVVAIASNNGGKQALETVQALLPVLCQAHGLTPEQVVAIASHDGG
KQALETVQALLPVLCQAHGLTPEQVVAIASHDGGKQALETVQALLPVLCQAHGLTPEQ
VVAIASNGGGKQALETVQRLLPVLCQAHGLTPEQVVAIASNIGGKQALETVQALLPVL
CQAHGLTPEQVVAIASNNGGKQALETVQRLLPVLCQAHGLTPEQVVAIASHDGGKQA
LETVQALLPVLCQAHGLTPEQVVAIASHDGGKQALETVQALLPVLCQAHGLTPEQVVA
IASNNGGKQALETVQALLPVLCQAHGLTPEQVVAIASNGGGKQALETVQRLLPVLCQA
HGLTPQQVVAIASNGGGKQALETVQRLLPVLCQAHGLTPQQVVAIASNGGGRPALESI
VAQLSRPDPALAALTNDHLVALACLGGRPALDAVKKGLG
ND2-DdCBE Right mitoTALE repeat
(SEQ ID NO: 364)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNIGGKQALETVQALLPVLCQAHGL
TPQQVVAIASNNGGKQALETVQRLLPVLCQAHGLTPQQVVAIASNIGGKQALETVQRL
LPVLCQAHGLTPEQVVAIASNIGGKQALETVQALLPVLCQAHGLTPEQVVAIASHDGG
KQALETVQALLPVLCQAHGLTPEQVVAIASNGGGKQALETVQALLPVLCQAHGLTPEQ
VVAIASNNGGKQALETVQRLLPVLCQAHGLTPEQVVAIASHDGGKQALETVQALLPVL
CQAHGLTPEQVVAIASNGGGKQALETVQRLLPVLCQAHGLTPEQVVAIASNIGGKQAL
ETVQALLPVLCQAHGLTPEQVVAIASNGGGKQALETVQALLPVLCQAHGLTPEQVVAI
ASNGGGKQALETVQALLPVLCQAHGLTPEQVVAIASNIGGKQALETVQRLLPVLCQAH
GLTPQQVVAIASNGGGKQALETVQRLLPVLCQAHGLTPQQVVAIASNGGGRPALESIV
AQLSRPDPALAALTNDHLVALACLGGRPALDAVKKGLG
ND2-DdCBE Left mitoTALE repeat
(SEQ ID NO: 365)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNGGGKQALETVQRLLPVLCQAHGLTPQQVVAIASNGGGKQALETVQR
LLPVLCQAHGLTPEQVVAIASNIGGKQALETVQALLPVLCQAHGLTPEQVVAIASNNG
GKQALETVQALLPVLCQAHGLTPEQVVAIASHDGGKQALETVQALLPVLCQAHGLTPE
QVVAIASNIGGKQALETVQRLLPVLCQAHGLTPEQVVAIASNGGGKQALETVQALLPV
LCQAHGLTPEQVVAIASNIGGKQALETVQRLLPVLCQAHGLTPEQVVAIASHDGGKQA
LETVQALLPVLCQAHGLTPEQVVAIASNGGGKQALETVQALLPVLCQAHGLTPEQVVA
IASHDGGKQALETVQALLPVLCQAHGLTPEQVVAIASHDGGKQALETVQRLLPVLCQA
HGLTPQQVVAIASNGGGKQALETVQRLLPVLCQAHGLTPEQVVAIASHDGGKQALETV
QALLPVLCQAHGLTPEQVVAIASNIGGKQALETVQALLPVLCQAHGLTPEQVVAIASNI
GGKQALETVQALLPVLCQAHGLTPQQVVAIASNGGGRPALESIVAQLSRPDPALAALT
NDHLVALACLGGRPALDAVKKGLG
ND4-DdCBE Right mitoTALE repeat
(SEQ ID NO: 366)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASHDGGKQALETVQRLLPVLCQAHGLTPQQVVAIASNGGGKQALETVQR
LLPVLCQAHGLTPEQVVAIASNNGGKQALETVQALLPVLCQAHGLTPEQVVAIASNGG
GKQALETVQALLPVLCQAHGLTPEQVVAIASNIGGKQALETVQALLPVLCQAHGLTPE
QVVAIASNIGGKQALETVQRLLPVLCQAHGLTPEQVVAIASNNGGKQALETVQALLPV
LCQAHGLTPEQVVAIASNGGGKQALETVQRLLPVLCQAHGLTPEQVVAIASNIGGKQA
LETVQRLLPVLCQAHGLTPEQVVAIASNNGGKQALETVQALLPVLCQAHGLTPEQVVA
IASNNGGKQALETVQALLPVLCQAHGLTPEQVVAIASNIGGKQALETVQALLPVLCQA
HGLTPEQVVAIASNNGGKQALETVQRLLPVLCQAHGLTPQQVVAIASNIGGKQALETV
QRLLPVLCQAHGLTPQQVVAIASNNGGKQALETVQRLLPVLCQAHGLTPQQVVAIASN
GGGRPALESIVAQLSRPDPALAALTNDHLVALACLGGRPALDAVKKGLG
ND4-DdCBE Left mitoTALE repeat
(SEQ ID NO: 367)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNNGGKQALETVQALLPVLCQAHGL
TPQQVVAIASHDGGKQALETVQRLLPVLCQAHGLTPQQVVAIASNGGGKQALETVQR
LLPVLCQAHGLTPEQVVAIASNIGGKQALETVQALLPVLCQAHGLTPEQVVAIASNNG
GKQALETVQALLPVLCQAHGLTPEQVVAIASNGGGKQALETVQALLPVLCQAHGLTPE
QVVAIASNIGGKQALETVQRLLPVLCQAHGLTPEQVVAIASNIGGKQALETVQALLPVL
CQAHGLTPEQVVAIASHDGGKQALETVQRLLPVLCQAHGLTPEQVVAIASHDGGKQA
LETVQALLPVLCQAHGLTPEQVVAIASNIGGKQALETVQALLPVLCQAHGLTPEQVVAI
ASHDGGKQALETVQALLPVLCQAHGLTPEQVVAIASNNGGKQALETVQRLLPVLCQA
HGLTPQQVVAIASNGGGKQALETVQRLLPVLCQAHGLTPEQVVAIASNGGGKQALETV
QALLPVLCQAHGLTPEQVVAIASHDGGKQALETVQALLPVLCQAHGLTPQQVVAIASN
GGGRPALESIVAQLSRPDPALAALTNDHLVALACLGGRPALDAVKKGLG
ND5.1-DdCBE Right mitoTALE repeat
(SEQ ID NO: 368)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASHDGGKQALETVQRLLPVLCQAHGL
TPEQVVAIASNGGGKQALETVQRLLPVLCQAHGLTPQQVVAIASNGGGKQALETVQRL
LPVLCQAHGLTPEQVVAIASNGGGKQALETVQALLPVLCQAHGLTPQQVVAIASNNGG
KQALETVQRLLPVLCQAHGLTPQQVVAIASNNGGKQALETVQRLLPVLCQAHGLTPEQ
VVAIASNIGGKQALETVQRLLPVLCQAHGLTPQQVVAIASNNGGKQALETVQRLLPVL
CQAHGLTPQQVVAIASNGGGKQALETVQRLLPVLCQAHGLTPEQVVAIASNIGGKQAL
ETVQRLLPVLCQAHGLTPEQVVAIASNNGGKQALETVQRLLPVLCQAHGLTPQQVVAI
ASNIGGKQALETVQRLLPVLCQAHGLTPQQVVAIASNIGGRPALESIVAQLSRPDPALA
ALTNDHLVALACLGGRPALDAVKKGLG
ND5.1-DdCBE Left mitoTALE repeat
(SEQ ID NO: 369)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNIGGKQALETVQALLPVLCQAHGL
TPQQVVAIASNNGGKQALETVQRLLPVLCQAHGLTPQQVVAIASHDGGKQALETVQR
LLPVLCQAHGLTPEQVVAIASNIGGKQALETVQALLPVLCQAHGLTPEQVVAIASNGG
GKQALETVQALLPVLCQAHGLTPEQVVAIASNGGGKQALETVQALLPVLCQAHGLTPE
QVVAIASNIGGKQALETVQRLLPVLCQAHGLTPEQVVAIASNNGGKQALETVQALLPV
LCQAHGLTPEQVVAIASHDGGKQALETVQRLLPVLCQAHGLTPEQVVAIASNIGGKQA
LETVQALLPVLCQAHGLTPEQVVAIASNNGGKQALETVQALLPVLCQAHGLTPEQVVA
IASNNGGKQALETVQALLPVLCQAHGLTPEQVVAIASNIGGKQALETVQRLLPVLCQA
HGLTPQQVVAIASNIGGKQALETVQRLLPVLCQAHGLTPQQVVAIASNGGGRPALESIV
AQLSRPDPALAALTNDHLVALACLGGRPALDAVKKGLG
ND5.2-DdCBE Right mitoTALE repeat (Note: Terminal NG RVD recognizes a
mismatched T instead of a G in the reference genome)
(SEQ ID NO: 370)
DIADLRTLGYSQQQQEKIKPKVRSTVAQHHEALVGHGFTHAHIVALSQHPAALG
TVAVKYQDMIAALPEATHEAIVGVGKQWSGARALEALLTVAGELRGPPLQLDTGQLL
KIAKRGGVTAVEAVHAWRNALTGAPLNLTPQQVVAIASNNGGKQALETVQRLLPVLC
QAHGLTPEQVVAIASHDGGKQALETVQRLLPVLCQAHGLTPQQVVAIASNGGGKQAL
ETVQRLLPVLCQAHGLTPEQVVAIASNIGGKQALETVQALLPVLCQAHGLTPQQVVAI
ASNNGGKQALETVQRLLPVLCQAHGLTPQQVVAIASNNGGKQALETVQRLLPVLCQA
HGLTPEQVVAIASHDGGKQALETVQRLLPVLCQAHGLTPQQVVAIASNGGGKQALETV
QRLLPVLCQAHGLTPQQVVAIASNNGGKQALETVQRLLPVLCQAHGLTPEQVVAIASH
DGGKQALETVQRLLPVLCQAHGLTPEQVVAIASHDGGKQALETVQRLLPVLCQAHGL
TPEQVVAIASNIGGKQALETVQALLPVLCQAHGLTPEQVVAIASNIGGKQALETVQALL
PVLCQAHGLTPQQVVAIASNGGGKQALETVQRLLPVLCQAHGLTPQQVVAIASNNGG
KQALETVQRLLPVLCQAHGLTPQQVVAIASNGGGRPALESIVAQLSRPDPALAALTND
HLVALACLGGRPALDAVKKGLG
ND5.2-DdCBE Left mitoTALE repeat
(SEQ ID NO: 371)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNIGGKQALETVQRLLPVLCQAHGLTPQQVVAIASNIGGKQALETVQRLL
PVLCQAHGLTPEQVVAIASNIGGKQALETVQALLPVLCQAHGLTPEQVVAIASNIGGKQ
ALETVQALLPVLCQAHGLTPEQVVAIASHDGGKQALETVQALLPVLCQAHGLTPEQVV
AIASHDGGKQALETVQRLLPVLCQAHGLTPEQVVAIASNIGGKQALETVQALLPVLCQ
AHGLTPEQVVAIASNGGGKQALETVQRLLPVLCQAHGLTPEQVVAIASNIGGKQALET
VQALLPVLCQAHGLTPEQVVAIASHDGGKQALETVQALLPVLCQAHGLTPEQVVAIAS
HDGGKQALETVQALLPVLCQAHGLTPEQVVAIASNGGGKQALETVQRLLPVLCQAHG
LTPQQVVAIASHDGGKQALETVQRLLPVLCQAHGLTPQQVVAIASNGGGRPALESIVA
QLSRPDPALAALTNDHLVALACLGGRPALDAVKKGLG
ND5.3-DdCBE Right mitoTALE repeat
(SEQ ID NO: 372)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASNIGGKQALETVQRLLPVLCQAHGL
TPEQVVAIASNIGGKQALETVQRLLPVLCQAHGLTPQQVVAIASNNGGKQALETVQRL
LPVLCQAHGLTPEQVVAIASNIGGKQALETVQALLPVLCQAHGLTPQQVVAIASNGGG
KQALETVQRLLPVLCQAHGLTPQQVVAIASNGGGKQALETVQRLLPVLCQAHGLTPEQ
VVAIASNIGGKQALETVQRLLPVLCQAHGLTPQQVVAIASNNGGKQALETVQRLLPVL
CQAHGLTPQQVVAIASNGGGKQALETVQRLLPVLCQAHGLTPEQVVAIASNIGGKQAL
ETVQRLLPVLCQAHGLTPEQVVAIASNGGGKQALETVQRLLPVLCQAHGLTPQQVVAI
ASNNGGKQALETVQRLLPVLCQAHGLTPQQVVAIASNNGGKQALETVQRLLPVLCQA
HGLTPQQVVAIASNGGGRPALESIVAQLSRPDPALAALTNDHLVALACLGGRPALDAV
KKGLG
ND5.3-DdCBE Left mitoTALE repeat
(SEQ ID NO: 373)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNIGGKQALETVQRLLPVLCQAHGLTPQQVVAIASNNGGKQALETVQRL
LPVLCQAHGLTPEQVVAIASNGGGKQALETVQALLPVLCQAHGLTPEQVVAIASNGGG
KQALETVQALLPVLCQAHGLTPEQVVAIASHDGGKQALETVQALLPVLCQAHGLTPEQ
VVAIASNGGGKQALETVQRLLPVLCQAHGLTPEQVVAIASNGGGKQALETVQALLPVL
CQAHGLTPEQVVAIASHDGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIA
SNIGGKQALETVQALLPVLCQAHGLTPEQVVAIASNGGGKQALETVQRLLPVLCQAHG
LTPQQVVAIASNIGGKQALETVQRLLPVLCQAHGLTPQQVVAIASNGGGRPALESIVAQ
LSRPDPALAALTNDHLVALACLGGRPALDAVKKGLG
ATP8-DdCBE Right mitoTALE repeat (Note: Terminal NG RVD recognizes a
mismatched T instead of a C in the reference genome)
(SEQ ID NO: 374)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASNIGGKQALETVQRLLPVLCQAHGL
TPEQVVAIASNGGGKQALETVQRLLPVLCQAHGLTPQQVVAIASNNGGKQALETVQRL
LPVLCQAHGLTPEQVVAIASNNGGKQALETVQALLPVLCQAHGLTPQQVVAIASNNGG
KQALETVQRLLPVLCQAHGLTPQQVVAIASHDGGKQALETVQRLLPVLCQAHGLTPEQ
VVAIASNGGGKQALETVQRLLPVLCQAHGLTPQQVVAIASNGGGKQALETVQRLLPVL
CQAHGLTPQQVVAIASNGGGKQALETVQRLLPVLCQAHGLTPEQVVAIASNNGGKQA
LETVQRLLPVLCQAHGLTPEQVVAIASNNGGKQALETVQRLLPVLCQAHGLTPQQVVA
IASNGGGRPALESIVAQLSRPDPALAALTNDHLVALACLGGRPALDAVKKGLG
ATP8-DdCBE Left mitoTALE repeat
(SEQ ID NO: 375)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNIGGKQALETVQALLPVLCQAHGL
TPQQVVAIASNGGGKQALETVQRLLPVLCQAHGLTPQQVVAIASNGGGKQALETVQR
LLPVLCQAHGLTPEQVVAIASNIGGKQALETVQALLPVLCQAHGLTPEQVVAIASNIGG
KQALETVQALLPVLCQAHGLTPEQVVAIASNIGGKQALETVQALLPVLCQAHGLTPEQ
VVAIASHDGGKQALETVQRLLPVLCQAHGLTPEQVVAIASNIGGKQALETVQALLPVL
CQAHGLTPEQVVAIASHDGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIA
SNIGGKQALETVQALLPVLCQAHGLTPEQVVAIASHDGGKQALETVQRLLPVLCQAHG
LTPQQVVAIASNGGGKQALETVQRLLPVLCQAHGLTPEQVVAIASNIGGKQALETVQA
LLPVLCQAHGLTPQQVVAIASNGGGRPALESIVAQLSRPDPALAALTNDHLVALACLG
GRPALDAVKKGLG

mtDNA BEs Comprising mitoZFs

The mtDNA base editors described herein contemplate fusion proteins comprising a mitoZF and a DddA domain or fragment or portion thereof (e.g., an N-terminal or C-terminal fragment or portion of a DddA), and optionally the joining of the two by a linker. The application contemplates any suitable mitoZF and a DddA domain to be combined in a single fusion protein. Examples of mitoZFs and DddA domains are each defined herein.

In some embodiments, the mtDNA base editors comprise DddA domains which are DdCBE, i.e., DddA which deaminates a C. Examples of general architecture of mtDNA base editors comprising DdCBEs and mitoZFs and their amino acid and nucleotide sequences are as follows:

| | | |
|---|---|---|
| R8 v6 | MLGFVGRVAAAPASGALRRLTPS | MTS |
| | ASLPPAQLLLRAAPTAVHPVRDY | MLGFVGRVAAAPASGALRRLTPSASLP |
| | AAQDYKDDDDKVDEMTKKFGT | PAQLLLRAAPTAVHPVRDYAAQ (SEQ |
| | LTIHDTEKGSLQKKLEELELDAA | ID NO: 394) |
| | MAERPFQCDICMRNFSTSGSLSR | FLAG tag |
| | HIRTHTGEKPFQCDICMRNFSQSG | DYKDDDDK (SEQ ID NO: 395) |
| | SLTRHIRTHTGSEKPFQCDICMRN | NES |
| | FSRSDALSQHIRTHTGEKPFQCDI | VDEMTKKFGTLTIHDTEK (SEQ ID NO: |
| | CMRNFSRNDNRITHIRTHTGEKPF | 396) |
| | QCDICMRNFSRSDHLTQHTKIHL | Linker |
| | RGSGGGGSGGSGGSGSYALGPY | GS |
| | QISAPQLPAYNGQTVGTFYYVND | NES2 |
| | AGGLESKVFSSGGPTPYPNYANA | LQKKLEELELD (SEQ ID NO: 397) |
| | GHVEGQSALFMRDNGISEGLVFH | Linker |
| | NNPEGTCGFCVNMTETLLPENAK | AA |
| | MTVVPPEGSGGSTNLSDIIEKETG | ZF (R8) |
| | KQLVIQESILMLPEEVEEVIGNKP | MAERPFQCDICMRNFSTSGSLSRHIRTH |
| | ESDILVHTAYDESTDENVMLLTS | TGEKPFQCDICMRNFSQSGSLTRHIRTH |
| | DAPEYKPWALVIQDSNGENKIKM | TGSEKPFQCDICMRNFSRSDALSQHIRT |
| | LGSGATNFSLLKQAGDVEENPGP | HTGEKPFQCDICMRNFSRNDNRITHIRT |
| | MASVLTPLLLRGLTGSARRLPVP | HTGEKPFQCDICMRNFSRSDHLTQHTKI |
| | RAKIHSLGSTNLSDIIEKETGKQL | HLR (SEQ ID NO: 398) |
| | VIQESILMLPEEVEEVIGNKPESDI | Linker |
| | LVHTAYDESTDENVMLLTSDAPE | GSGGGGSGGSGGS (SEQ ID NO: 399) |
| | YKPWALVIQDSNGENKIKML | Split DddA (DddA-G1397N) |
| | (SEQ ID NO: 382) | GSYALGPYQISAPQLPAYNGQTVGTFY |
| | | YVNDAGGLESKVFSSGGPTPYPNYANA |
| | | GHVEGQSALFMRDNGISEGLVFHNNPE |
| | | GTCGFCVNMTETLLPENAKMTVVPPE |
| | | G (SEQ ID NO: 284) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| | | Linker |
| | | GSG |
| | | P2A Peptide |
| | | ATNFSLLKQAGDVEENPGP (SEQ ID |
| | | NO: 400) |
| | | MTS |
| | | MASVLTPLLLRGLTGSARRLPVPRAKI |
| | | HSL (SEQ ID NO: 401) |
| | | Linker |
| | | GS |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| 5xZnF- | MLGFVGRVAAAPASGALRRLTPS | MTS |
| 4-R8 v6 | ASLPPAQLLLRAAPTAVHPVRDY | MLGFVGRVAAAPASGALRRLTPSASLP |
| | AAQDYKDDDDKVDEMTKKFGT | PAQLLLRAAPTAVHPVRDYAAQ (SEQ |
| | LTIHDTEKGSLQKKLEELELDAA | ID NO: 394) |
| | MAERPFQCDICMRNFSQASNLIS | FLAG tag |
| | HIRTHTGEKPFQCDICMRNFSTSH | DYKDDDDK (SEQ ID NO: 395) |
| | SLTEHIRTHTGSEKPFQCDICMRN | NES |
| | FSERSHLREHIRTHTGEKPFQCDI | VDEMTKKFGTLTIHDTEK (SEQ ID NO: |
| | CMRNFSQSGNLTEHIRTHTGEKP | 396) |
| | FQCDICMRNFSSKKALTEHTKIHL | Linker |
| | RGSGGGGSGGSGGSAIPVKRGAT | GS |
| | GETKVFTGNSNSPKSPTKGGCSG | NES2 |
| | GSTNLSDIIEKETGKQLVIQESILM | LQKKLEELELD (SEQ ID NO: 397) |
| | LPEEVEEVIGNKPESDILVHTAYD | Linker |
| | ESTDENVMLLTSDAPEYKPWAL | AA |
| | VIQDSNGENKIKMLGSGATNESL | ZF (5xZnF-4-R8) |
| | LKQAGDVEENPGPMASVLTPLLL | MAERPFQCDICMRNFSQASNLISHIRTH |
| | RGLTGSARRLPVPRAKIHSLGSTN | TGEKPFQCDICMRNFSTSHSLTEHIRTH |
| | LSDIIEKETGKQLVIQESILMLPEE | TGSEKPFQCDICMRNFSERSHLREHIRT |
| | VEEVIGNKPESDILVHTAYDESTD | HTGEKPFQCDICMRNFSQSGNLTEHIRT |
| | ENVMLLTSDAPEYKPWALVIQDS | HTGEKPFQCDICMRNFSSKKALTEHTKI |
| | NGENKIKML (SEQ ID NO: 383) | HLR (SEQ ID NO: 402) |
| | | Linker |
| | | GSGGGGSGGSGGS (SEQ ID NO: 399) |
| | | Split DddA (DddA-G1397C) |
| | | AIPVKRGATGETKVFTGNSNSPKSPTK |
| | | GGC (SEQ ID NO: 286) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| | | Linker |
| | | GSG |
| | | P2A Peptide |
| | | ATNFSLLKQAGDVEENPGP (SEQ ID |
| | | NO: 400) |
| | | MTS |
| | | MASVLTPLLLRGLTGSARRLPVPRAKI |
| | | HSL (SEQ ID NO: 401) |
| | | Linker |
| | | GS |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| 5xZnF- | MLGFVGRVAAAPASGALRRLTPS | MTS |
| 10-R8 | ASLPPAQLLLRAAPTAVHPVRDY | MLGFVGRVAAAPASGALRRLTPSASLP |
| v6 | AAQDYKDDDDKVDEMTKKFGT | PAQLLLRAAPTAVHPVRDYAAQ (SEQ |
| | LTIHDTEKGSLQKKLEELELDAA | ID NO: 394) |
| | MAERPFQCDICMRNFSQASNLIS | FLAG tag |
| | HIRTHTGEKPFQCDICMRNFSQR | DYKDDDDK (SEQ ID NO: 395) |
| | ANLRAHIRTHTGSEKPFQCDICM | NES |
| | RNFSQASNLISHIRTHTGEKPFQC | VDEMTKKFGTLTIHDTEK (SEQ ID NO: |
| | DICMRNFSTSHSLTEHIRTHTGEK | 396) |
| | PFQCDICMRNFSERSHLREHTKIH | Linker |
| | LRGSGGGGSGGSGGSAIPVKRGA | GS |
| | TGETKVFTGNSNSPKSPTKGGCS | NES2 |
| | GGSTNLSDIIEKETGKQLVIQESIL | Linker |
| | MLPEEVEEVIGNKPESDILVHTAY | LQKKLEELELD (SEQ ID NO: 397) |
| | DESTDENVMLLTSDAPEYKPWA | AA |
| | LVIQDSNGENKIKMLGSGATNFS | ZF (5xZnF-10-R8) |
| | LLKQAGDVEENPGPMASVLTPLL | MAERPFQCDICMRNFSQASNLISHIRTH |
| | LRGLTGSARRLPVPRAKIHSLGST | TGEKPFQCDICMRNFSQRANLRAHIRT |
| | NLSDIIEKETGKQLVIQESILMLPE | HTGSEKPFQCDICMRNFSQASNLISHIR |
| | EVEEVIGNKPESDILVHTAYDEST | THTGEKPFQCDICMRNFSTSHSLTEHIR |
| | DENVMLLTSDAPEYKPWALVIQ | THTGEKPFQCDICMRNFSERSHLREHT |
| | DSNGENKIKML (SEQ ID NO: 384) | KIHLR (SEQ ID NO: 403) |
| | | Linker |
| | | GSGGGGSGGSGGS (SEQ ID NO: 399) |
| | | Split DddA (DddA-G1397C) |
| | | AIPVKRGATGETKVFTGNSNSPKSPTK |
| | | GGC (SEQ ID NO: 286) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| | | Linker |
| | | GSG |
| | | P2A Peptide |
| | | ATNFSLLKQAGDVEENPGP (SEQ ID |
| | | NO: 400) |
| | | MTS |
| | | MASVLTPLLLRGLTGSARRLPVPRAKI |
| | | HSL (SEQ ID NO: 401) |
| | | Linker |
| | | GS |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| R13-1 | MLGFVGRVAAAPASGALRRLTPS | MTS |
| v6 | ASLPPAQLLLRAAPTAVHPVRDY | MLGFVGRVAAAPASGALRRLTPSASLP |
| | AAQDYKDDDDKVDEMTKKFGT | PAQLLLRAAPTAVHPVRDYAAQ (SEQ |
| | LTIHDTEKGSLQKKLEELELDAA | ID NO: 394) |
| | MAERPFQCDICMRNFSRSDNLST | FLAG tag |
| | HIRTHTGEKPFQCDICMRNFSDRS | DYKDDDDK (SEQ ID NO: 395) |
| | DLSRHIRTHTGEKPFQCDICMRNF | NES |
| | SQSGDLTRHIRTHTGSEKPFQCDI | VDEMTKKFGTLTIHDTEK (SEQ ID NO: |
| | CMRNFSRSDSLSAHIRTHTGEKPF | 396) |
| | QCDICMRNFSQKATRITHTKIHLR | Linker |
| | GSGGGGSGGSGGSGSYALGPYQI | GS |
| | SAPQLPAYNGQTVGTFYYVNDA | NES2 |
| | GGLESKVFSSGGPTPYPNYANAG | LQKKLEELELD (SEQ ID NO: 397) |
| | HVEGQSALFMRDNGISEGLVFHN | Linker |
| | NPEGTCGFCVNMTETLLPENAK | AA |
| | MTVVPPEGSGGSTNLSDIIEKETG | ZF (R13-1) |
| | KQLVIQESILMLPEEVEEVIGNKP | MAERPFQCDICMRNFSRSDNLSTHIRTH |
| | ESDILVHTAYDESTDENVMLLTS | TGEKPFQCDICMRNFSDRSDLSRHIRTH |
| | DAPEYKPWALVIQDSNGENKIKM | TGEKPFQCDICMRNFSQSGDLTRHIRTH |
| | LGSGATNFSLLKQAGDVEENPGP | TGSEKPFQCDICMRNFSRSDSLSAHIRT |
| | MASVLTPLLLRGLTGSARRLPVP | HTGEKPFQCDICMRNFSQKATRITHTKI |
| | RAKIHSLGSTNLSDIIEKETGKQL | HLR (SEQ ID NO: 404) |
| | VIQESILMLPEEVEEVIGNKPESDI | Linker |
| | LVHTAYDESTDENVMLLTSDAPE | GSGGGGSGGSGGS (SEQ ID NO: 399) |
| | YKPWALVIQDSNGENKIKML | Split DddA (DddA-G1397N) |
| | (SEQ ID NO: 385) | GSYALGPYQISAPQLPAYNGQTVGTFY |
| | | YVNDAGGLESKVFSSGGPTPYPNYANA |
| | | GHVEGQSALFMRDNGISEGLVFHNNPE |
| | | GTCGFCVNMTETLLPENAKMTVVPPE |
| | | G (SEQ ID NO: 284) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| | | Linker |
| | | GSG |
| | | P2A Peptide |
| | | ATNFSLLKQAGDVEENPGP (SEQ ID |
| | | NO: 400) |
| | | MTS |
| | | MASVLTPLLLRGLTGSARRLPVPRAKI |
| | | HSL (SEQ ID NO: 401) |
| | | Linker |
| | | GS |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| 5xZnF- | MLGFVGRVAAAPASGALRRLTPS | MTS |
| 9-R13 | ASLPPAQLLLRAAPTAVHPVRDY | MLGFVGRVAAAPASGALRRLTPSASLP |
| v6 | AAQDYKDDDDKVDEMTKKFGT | PAQLLLRAAPTAVHPVRDYAAQ (SEQ |
| | LTIHDTEKGSLQKKLEELELDAA | ID NO: 394) |
| | MAERPFQCDICMRNFSQSSSLVR | FLAG tag |
| | HIRTHTGEKPFQCDICMRNFSRSD | DYKDDDDK (SEQ ID NO: 395) |
| | NLVRHIRTHTGSEKPFQCDICMR | NES |
| | NFSQAGHLASHIRTHTGEKPFQC | VDEMTKKFGTLTIHDTEK (SEQ ID NO: |
| | DICMRNFSRKDNLKNHIRTHTGE | 396) |
| | KPFQCDICMRNFSRKDALRGHTK | Linker |
| | IHLRGSGGGGSGGSGGSAIPVKR | GS |
| | GATGETKVFTGNSNSPKSPTKGG | NES2 |
| | CSGGSTNLSDIIEKETGKQLVIQES | LQKKLEELELD (SEQ ID NO: 397) |
| | ILMLPEEVEEVIGNKPESDILVHT | Linker |
| | AYDESTDENVMLLTSDAPEYKP | AA |
| | WALVIQDSNGENKIKMLGSGAT | ZF (5xZnF-9-R13) |
| | NFSLLKQAGDVEENPGPMASVLT | MAERPFQCDICMRNFSQSSSLVRHIRTH |
| | PLLLRGLTGSARRLPVPRAKIHSL | TGEKPFQCDICMRNFSRSDNLVRHIRTH |
| | GSTNLSDIIEKETGKQLVIQESILM | TGSEKPFQCDICMRNFSQAGHLASHIRT |
| | LPEEVEEVIGNKPESDILVHTAYD | HTGEKPFQCDICMRNFSRKDNLKNHIR |
| | ESTDENVMLLTSDAPEYKPWAL | THTGEKPFQCDICMRNFSRKDALRGHT |
| | VIQDSNGENKIKML (SEQ ID NO: | KIHLR (SEQ ID NO: 405) |
| | 386) | Linker |
| | | GSGGGGSGGSGGS (SEQ ID NO: 399) |
| | | Split DddA (DddA-G1397C) |
| | | AIPVKRGATGETKVFTGNSNSPKSPTK |
| | | GGC (SEQ ID NO: 286) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| | | Linker |
| | | GSG |
| | | P2A Peptide |
| | | ATNFSLLKQAGDVEENPGP (SEQ ID |
| | | NO: 400) |
| | | MTS |
| | | MASVLTPLLLRGLTGSARRLPVPRAKI |
| | | HSL (SEQ ID NO: 401) |
| | | Linker |
| | | GS |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| 5xZnF- | MLGFVGRVAAAPASGALRRLTPS | MTS |
| 12-R13 | ASLPPAQLLLRAAPTAVHPVRDY | MLGFVGRVAAAPASGALRRLTPSASLP |
| v6 | AAQDYKDDDDKVDEMTKKFGT | PAQLLLRAAPTAVHPVRDYAAQ (SEQ |
| | LTIHDTEKGSLQKKLEELELDAA | ID NO: 394) |
| | MAERPFQCDICMRNFSRSDHLTT | FLAG tag |
| | HIRTHTGEKPFQCDICMRNFSQSS | DYKDDDDK (SEQ ID NO: 395) |
| | SLVRHIRTHTGSEKPFQCDICMRN | NES |
| | FSRSDNLVRHIRTHTGEKPFQCDI | VDEMTKKFGTLTIHDTEK (SEQ ID NO: |
| | CMRNFSQAGHLASHIRTHTGEKP | 396) |
| | FQCDICMRNFSRKDNLKNHTKIH | Linker |
| | LRGSGGGGSGGSGGSAIPVKRGA | GS |
| | TGETKVFTGNSNSPKSPTKGGCS | NES2 |
| | GGSTNLSDIIEKETGKQLVIQESIL | LQKKLEELELD (SEQ ID NO: 397) |
| | MLPEEVEEVIGNKPESDILVHTAY | Linker |
| | DESTDENVMLLTSDAPEYKPWA | AA |
| | LVIQDSNGENKIKMLGSGATNFS | ZF (5xZnF-12-R13) |
| | LLKQAGDVEENPGPMASVLTPLL | MAERPFQCDICMRNFSRSDHLTTHIRT |
| | LRGLTGSARRLPVPRAKIHSLGST | HTGEKPFQCDICMRNFSQSSSLVRHIRT |
| | NLSDIIEKETGKQLVIQESILMLPE | HTGSEKPFQCDICMRNFSRSDNL VRHIR |
| | EVEEVIGNKPESDILVHTAYDEST | THTGEKPFQCDICMRNFSQAGHLASHI |
| | DENVMLLTSDAPEYKPWALVIQ | RTHTGEKPFQCDICMRNFSRKDNLKNH |
| | DSNGENKIKML (SEQ ID NO: 387) | TKIHLR (SEQ ID NO: 406) |
| | | Linker |
| | | GSGGGGSGGSGGS (SEQ ID NO: 399) |
| | | Split DddA (DddA-G1397C) |
| | | AIPVKRGATGETKVFTGNSNSPKSPTK |
| | | GGC (SEQ ID NO: 286) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| | | Linker |
| | | GSG |
| | | P2A Peptide |
| | | ATNFSLLKQAGDVEENPGP (SEQ ID |
| | | NO: 400) |
| | | MTS |
| | | MASVLTPLLLRGLTGSARRLPVPRAKI |
| | | HSL (SEQ ID NO: 401) |
| | | Linker |
| | | GS |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| R8 v3 | MLGFVGRVAAAPASGALRRLTPS | MTS |
| | ASLPPAQLLLRAAPTAVHPVRDY | MLGFVGRVAAAPASGALRRLTPSASLP |
| | AAQDYKDDDDKVDEMTKKFGT | PAQLLLRAAPTAVHPVRDYAAQ (SEQ |
| | LTIHDTEKAAMAERPFQCRICMR | ID NO: 394) |
| | NFSTSGSLSRHIRTHTGEKPFACDI | FLAG tag |
| | CGRKFAQSGSLTRHTKIHTGGQR | DYKDDDDK (SEQ ID NO: 395) |
| | PFQCRICMRNFSRSDALSQHIRTH | NES |
| | TGEKPFACDICGRKFARNDNRIT | ZF (R8) |
| | HTKIHTGEKPFQCRICMRKFARS | VDEMTKKFGTLTIHDTEK (SEQ ID NO: |
| | DHLTQHTKIHLRGSGGGGSGGSG | 396) |
| | GSGSYALGPYQISAPQLPAYNGQ | Linker |
| | TVGTFYYVNDAGGLESKVFSSGG | AA |
| | PTPYPNYANAGHVEGQSALFMR | MAERPFQCRICMRNFSTSGSLSRHIRTH |
| | DNGISEGLVFHNNPEGTCGFCVN | TGEKPFACDICGRKFAQSGSLTRHTKIH |
| | MTETLLPENAKMTVVPPEGSGGS | TGGQRPFQCRICMRNFSRSDALSQHIRT |
| | TNLSDIIEKETGKQLVIQESILMLP | HTGEKPFACDICGRKFARNDNRITHTKI |
| | EEVEEVIGNKPESDILVHTAYDES | HTGEKPFQCRICMRKFARSDHLTQHTK |
| | TDENVMLLTSDAPEYKPWALVIQ | IHLR (SEQ ID NO: 407) |
| | DSNGENKIKML (SEQ ID NO: 388) | Linker |
| | | GSGGGGSGGSGGS (SEQ ID NO: 399) |
| | | Split DddA (DddA-G1397N) |
| | | GSYALGPYQISAPQLPAYNGQTVGTFY |
| | | YVNDAGGLESKVFSSGGPTPYPNYANA |
| | | GHVEGQSALFMRDNGISEGLVFHNNPE |
| | | GTCGFCVNMTETLLPENAKMTVVPPE |
| | | G (SEQ ID NO: 284) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| 5xZnF- | MLGFVGRVAAAPASGALRRLTPS | MTS |
| 4-R8 v3 | ASLPPAQLLLRAAPTAVHPVRDY | MLGFVGRVAAAPASGALRRLTPSASLP |
| | AAQDYKDDDDKVDEMTKKFGT | PAQLLLRAAPTAVHPVRDYAAQ (SEQ |
| | LTIHDTEKAAMAERPFQCRICMR | ID NO: 394) |
| | NFSQASNLISHIRTHTGEKPFACDI | FLAG tag |
| | CGRKFATSHSLTEHTKIHTGSQKP | DYKDDDDK (SEQ ID NO: 395) |
| | FQCRICMRNFSERSHLREHIRTHT | NES |
| | GEKPFACDICGRKFAQSGNLTEH | VDEMTKKFGTLTIHDTEK (SEQ ID NO: |
| | TKIHTGEKPFQCRICMRKFASKK | 396) |
| | ALTEHTKIHLRGSGGGGSGGSGG | Linker |
| | SAIPVKRGATGETKVFTGNSNSP | AA |
| | KSPTKGGCSGGSTNLSDIIEKETG | ZF (5xZnF-4-R8) |
| | KQLVIQESILMLPEEVEEVIGNKP | MAERPFQCRICMRNFSQASNLISHIRTH |
| | ESDILVHTAYDESTDENVMLLTS | TGEKPFACDICGRKFATSHSLTEHTKIH |
| | DAPEYKPWALVIQDSNGENKIKM | TGSQKPFQCRICMRNFSERSHLREHIRT |
| | L (SEQ ID NO: 389) | HTGEKPFACDICGRKFAQSGNLTEHTKI |
| | | HTGEKPFQCRICMRKFASKKALTEHTK |
| | | IHLR (SEQ ID NO: 408) |
| | | Linker |
| | | GSGGGGSGGSGGS (SEQ ID NO: 399) |
| | | Split DddA (DddA-G1397C) |
| | | AIPVKRGATGETKVFTGNSNSPKSPTK |
| | | GGC (SEQ ID NO: 286) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| 5xZnF- | MLGFVGRVAAAPASGALRRLTPS | MTS |
| 10-R8 | ASLPPAQLLLRAAPTAVHPVRDY | MLGFVGRVAAAPASGALRRLTPSASLP |
| v3 | AAQDYKDDDDKVDEMTKKFGT | PAQLLLRAAPTAVHPVRDYAAQ (SEQ |
| | LTIHDTEKAAMAERPFQCRICMR | ID NO: 394) |
| | NFSQASNLISHIRTHTGEKPFACDI | FLAG tag |
| | CGRKFAQRANLRAHTKIHTGSQK | DYKDDDDK (SEQ ID NO: 395) |
| | PFQCRICMRNFSQASNLISHIRTH | NES |
| | TGEKPFACDICGRKFATSHSLTEH | VDEMTKKFGTLTIHDTEK (SEQ ID NO: |
| | TKIHTGEKPFQCRICMRKFAERSH | 396) Linker |
| | LREHTKIHLRGSGGGGSGGSGGS | AA |
| | AIPVKRGATGETKVFTGNSNSPK | ZF (5xZnF-10-R8) |
| | SPTKGGCSGGSTNLSDIIEKETGK | MAERPFQCRICMRNFSQASNLISHIRTH |
| | QLVIQESILMLPEEVEEVIGNKPES | TGEKPFACDICGRKFAQRANLRAHTKI |
| | DILVHTAYDESTDENVMLLTSDA | HTGSQKPFQCRICMRNFSQASNLISHIR |
| | PEYKPWALVIQDSNGENKIKML | THTGEKPFACDICGRKFATSHSLTEHTK |
| | (SEQ ID NO: 390) | IHTGEKPFQCRICMRKFAERSHLREHTK |
| | | IHLR (SEQ ID NO: 409) |
| | | Linker |
| | | GSGGGGSGGSGGS (SEQ ID NO: 399) |
| | | Split DddA (DddA-G1397C) |
| | | AIPVKRGATGETKVFTGNSNSPKSPTK |
| | | GGC (SEQ ID NO: 286) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| R13-1 | MLGFVGRVAAAPASGALRRLTPS | MTS |
| v3 | ASLPPAQLLLRAAPTAVHPVRDY | MLGFVGRVAAAPASGALRRLTPSASLP |
| | AAQDYKDDDDKVDEMTKKFGT | PAQLLLRAAPTAVHPVRDYAAQ (SEQ |
| | LTIHDTEKAAMAERPFQCRICMR | ID NO: 394) |
| | NFSRSDNLSTHIRTHTGEKPFACD | FLAG tag |
| | ICGRKFADRSDLSRHTKIHTGEKP | DYKDDDDK (SEQ ID NO: 395) |
| | FQCRICMRKFAQSGDLTRHTKIH | NES |
| | TGSQKPFQCRICMRNFSRSDSLSA | VDEMTKKFGTLTIHDTEK (SEQ ID NO: |
| | HIRTHTGEKPFACDICGRKFAQK | 396) |
| | ATRITHTKIHLRGSGGGGSGGSG | Linker |
| | GSGSYALGPYQISAPQLPAYNGQ | AA |
| | TVGTFYYVNDAGGLESKVFSSGG | ZF (R13-1) |
| | PTPYPNYANAGHVEGQSALFMR | MAERPFQCRICMRNFSRSDNLSTHIRTH |
| | DNGISEGLVFHNNPEGTCGFCVN | TGEKPFACDICGRKFADRSDLSRHTKIH |
| | MTETLLPENAKMTVVPPEGSGGS | TGEKPFQCRICMRKFAQSGDLTRHTKI |
| | TNLSDIIEKETGKQLVIQESILMLP | HTGSQKPFQCRICMRNFSRSDSLSAHIR |
| | EEVEEVIGNKPESDILVHTAYDES | THTGEKPFACDICGRKFAQKATRITHT |
| | TDENVMLLTSDAPEYKPWALVIQ | KIHLR (SEQ ID NO: 410) |
| | DSNGENKIKML (SEQ ID NO: 391) | Linker |
| | | GSGGGGSGGSGGS (SEQ ID NO: 399) |
| | | Split DddA (DddA-G1397N) |
| | | GSYALGPYQISAPQLPAYNGQTVGTFY |
| | | YVNDAGGLESKVFSSGGPTPYPNYANA |
| | | GHVEGQSALFMRDNGISEGLVFHNNPE |
| | | GTCGFCVNMTETLLPENAKMTVVPPE |
| | | G (SEQ ID NO: 284) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| 5xZnF- | MLGFVGRVAAAPASGALRRLTPS | MTS |
| 9-R13 | ASLPPAQLLLRAAPTAVHPVRDY | MLGFVGRVAAAPASGALRRLTPSASLP |
| v3 | AAQDYKDDDDKVDEMTKKFGT | PAQLLLRAAPTAVHPVRDYAAQ (SEQ |
| | LTIHDTEKAAMAERPFQCRICMR | ID NO: 394) |
| | NFSQSSSLVRHIRTHTGEKPFACD | FLAG tag |
| | ICGRKFARSDNLVRHTKIHTGSQ | DYKDDDDK (SEQ ID NO: 395) |
| | KPFQCRICMRNFSQAGHLASHIR | NES |
| | THTGEKPFACDICGRKFARKDNL | VDEMTKKFGTLTIHDTEK (SEQ ID NO: |
| | KNHTKIHTGEKPFQCRICMRKFA | 396) |
| | RKDALRGHTKIHLRGSGGGGSGG | Linker |
| | SGGSAIPVKRGATGETKVFTGNS | AA |
| | NSPKSPTKGGCSGGSTNLSDIIEK | ZF (5xZnF-9-R13) |
| | ETGKQLVIQESILMLPEEVEEVIG | MAERPFQCRICMRNFSQSSSLVRHIRTH |
| | NKPESDILVHTAYDESTDENVML | TGEKPFACDICGRKFARSDNLVRHTKI |
| | LTSDAPEYKPWALVIQDSNGENK | HTGSQKPFQCRICMRNFSQAGHLASHI |
| | IKML (SEQ ID NO: 392) | RTHTGEKPFACDICGRKFARKDNLKNH |
| | | TKIHTGEKPFQCRICMRKFARKDALRG |
| | | HTKIHLR (SEQ ID NO: 411) |
| | | Linker |
| | | GSGGGGSGGSGGS (SEQ ID NO: 399) |
| | | Split DddA (DddA-G1397C) |
| | | AIPVKRGATGETKVFTGNSNSPKSPTK |
| | | GGC (SEQ ID NO: 286) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |
| 5xZnF- | MLGFVGRVAAAPASGALRRLTPS | MTS |
| 12-R13 | ASLPPAQLLLRAAPTAVHPVRDY | MLGFVGRVAAAPASGALRRLTPSASLP |
| v3 | AAQDYKDDDDKVDEMTKKFGT | PAQLLLRAAPTAVHPVRDYAAQ (SEQ |
| | LTIHDTEKAAMAERPFQCRICMR | ID NO: 394) |
| | NFSRSDHLTTHIRTHTGEKPFACD | FLAG tag |
| | ICGRKFAQSSSLVRHTKIHTGSQK | DYKDDDDK (SEQ ID NO: 395) |
| | PFQCRICMRNFSRSDNLVRHIRTH | NES |
| | TGEKPFACDICGRKFAQAGHLAS | VDEMTKKFGTLTIHDTEK (SEQ ID NO: |
| | HTKIHTGEKPFQCRICMRKFARK | 396) |
| | DNLKNHTKIHLRGSGGGGSGGSG | Linker |
| | GSAIPVKRGATGETKVFTGNSNS | AA |
| | PKSPTKGGCSGGSTNLSDIIEKET | ZF (5xZnF-12-R13) |
| | GKQLVIQESILMLPEEVEEVIGNK | MAERPFQCRICMRNFSRSDHLTTHIRTH |
| | PESDILVHTAYDESTDENVMLLT | TGEKPFACDICGRKFAQSSSLVRHTKIH |
| | SDAPEYKPWALVIQDSNGENKIK | TGSQKPFQCRICMRNFSRSDNLVRHIRT |
| | ML (SEQ ID NO: 393) | HTGEKPFACDICGRKFAQAGHLASHTK |
| | | IHTGEKPFQCRICMRKFARKDNLKNHT |
| | | KIHLR (SEQ ID NO: 412) |
| | | Linker |
| | | GSGGGGSGGSGGS (SEQ ID NO: 399) |
| | | Split DddA (DddA-G1397C) |
| | | AIPVKRGATGETKVFTGNSNSPKSPTK |
| | | GGC (SEQ ID NO:286) |
| | | Linker |
| | | SGGS (SEQ ID NO: 348) |
| | | UGI |
| | | TNLSDIIEKETGKQLVIQESILMLPEEVE |
| | | EVIGNKPESDILVHTAYDESTDENVML |
| | | LTSDAPEYKPWALVIQDSNGENKIKML |
| | | (SEQ ID NO: 341) |

### DddAs

In various embodiments, the mtDNA base editors or the polypeptides that comprise the mtDNA base editors (e.g., the pDNAbps and DddA) may be engineered to include a DddA, or an inactive fragment thereof.

In various embodiments, the DddA protein has the following amino acid sequence:

GSYALGPYQISAPQLPAYNGQTVGTFYYVNDAGGLESKVFSSGGPTPYPNYAN AGHVEGQSALFMRDNGISEGLVFHNNPEGTCGFCVNMTETLLPENAKMTVVPPEGAIP VKRGATGETKVFTGNSNSPKSPTKGGC (SEQ IN NO: 338), or an amino acid sequence having at least 70%, at least 75%, at least 80%, at least 85%, at least 90%, at least 95%, at least 96%, at least 97%, at least 98%, at least 99%, or 100% sequence identify with DddA of SEQ ID NO: 338, or a fragment thereof.

This full length DddA may also be referred to as “DddAtox” since it is toxic to cells, as described in Example 1.

In other embodiments, the DddA has the following amino acid sequence: XGSSHHHHHHSQDPIGLNGG ANVYHYAPNP VGWVDPWGLA GSYALGPYQI SAPQLPAYNGQTVGTFYYVN DAGGLESKVF SSGGPTPYPN YANAGHVEGQ SALFXRDNGI SEGLVFHNNPEGTCGFCVNX TETLLPENAK XTVVPPEGAI PVKRGATGET KVFTGNSNSPKSPTKGGC (SEQ ID NO: 413) (which corresponds to PDB Accession No. 6U08_A of Burkholderia cenocepacia), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of 6U08_A (SEQ ID NO: 413).

In various other embodiments, a split DddA can have the following sequences:
G1333 DddAtox-N¬GSYALGPYQISAPQLPAYNGQTVGTFYYVNDAGGLESKVFSSGG (SEQ ID NO: 349), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 349.

G1333 DddAtox-C PTPYPNYANAGHVEGQSALFMRDNGISEGLVFHNNPEGTCGFCVNMTETLLPENAKMT VVPPEGAIPVKRGATGETKVFTGNSNSPKSPTKGGC (SEQ ID NO: 350), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 350.

G1397 DddAtox-N¬GSYALGPYQISAPQLPAYNGQTVGTFYYVNDAGGLESKVFSSGGPTPYPNYANAGHVE GQSALFMRDNGISEGLVFHNNPEGTCGFCVNMTETLLPENAKMTVVPPEG (SEQ ID NO: 351), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 351.

G1397 DddAtox-C AIPVKRGATGETKVFTGNSNSPKSPTKGGC (SEQ ID NO: 352), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 352.

Split DddA (DddA-G1397N) GSYALGPYQISAPQLPAYNGQTVGTFYYVNDAGGLESKVFSSGGPTPYPNYANAGHVE GQSALFMRDNGISEGLVFHNNPEGTCGFCVNMTETLLPENAKMTVVPPEG (SEQ ID NO: 351), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 351.

Split DddA (DddA-G1397C)
(SEQ ID NO: 352)
AIPVKRGATGETKVFTGNSNSPKSPTKGGC.

The disclosure also contemplates the use of any variant of DddAtox, or proteins comprising an amino acid sequence having at least 70%, at least 75%, at least 80%, at least 85%, at least 90%, at least 95%, at least 96%, at least 97%, at least 98%, at least 99%, or 100% sequence identify with DddA-G1397C, or a biologically active fragment of DddA-G1397C.

As shown in FIG. 1A, the present inventors have recognized that the whole, intact DddA is toxic to cells. Thus, in order to utilize the DddA in the context of the mtDNA base editors described herein, the DddA must be delivered in an inactive form. One of ordinary skill in the art will appreciate that various methods, techniques, and modification known in the art can be adapted for reversibly inactivating DddA such that the enzyme may be delivered to a cell in an inactive state, but then become activated inside the cell (or the mitochondria) under one or more conditions, or in the presence of one or more inducing agents, in order to conduct the desired deamination.

In preferred embodiments, as depicted in FIGS. 1A-1F, the DddA may be split into inactive fragments which can be separately delivered to a target deamination site on separate fusion constructs that target each fragment of the DddA to sites positioned on either side of a target edit site.

In some embodiments, the DddA comprises a first portion and a second portion. In some embodiments, the first portion and the second portion together comprise a full length DddA. In some embodiments, the first and second portion comprise less than the full length DddA portion. In some embodiments, the first and second portion independently do not have any, or have minimal, native DddA activity (e.g., deamination activity). In some embodiment, the first and second portion can re-assemble (i.e., dimerize) into a DddA protein with, at least partial, native DddA activity (e.g., deamination activity).

In some embodiments, the first and second portion of the DddA are formed by truncating (i.e., dividing or splitting the DddA protein) at specified amino acid residues. In some embodiments, the first portion of a DddA comprises a full-length DddA truncated at its N-terminus. In some embodiments, the second portion of a DddA comprises a full-length DddA truncated at its C-terminus. In some embodiments, additional truncations are performed to either the full-length DddA or to the first or second portions of the DddA. In some embodiments, the first and second portions of a DddA may comprise additional truncations, but which the first and second portion can dimerize or re-assemble, to restore, at least partially, native DddA activity (e.g., deamination). In some embodiments, the first and second portions comprise full-length DddA truncated at, or around, a residue in DddA selected from the group comprising: 62, 71, 73, 84, 94, 108, 110, 122, 135, 138, 148, and 155. In some embodiments, the truncation of DddA occurs at residue 148.

In certain embodiments, the DddA can be separated into two fragments by dividing the DddA at a split site. A “split site” refers to a position between two adjacent amino acids (in a wildtype DddA amino acid sequence) that marks a point of division of a DddA. In certain embodiments, the DddA can have a least one split site, such that once divided at that split site, the DddA forms an N-terminal fragment and a C-terminal fragment. The N-terminal and C-terminal fragments can be the same or difference sizes (or lengths), wherein the size and/or polypeptide length depends on the location or position of the split site. As used herein, reference to a “fragment” of DddA (or any other polypeptide) can be referred equivalently as a “portion.” Thus, a DddA which is divided at a split site can form an N-terminal portion and a C-terminal portion. Preferably, the N-terminal fragment (or portion) and the C-terminal fragment (or portion) or DddA do not have a deaminase activity.

In various embodiments, a DddA may be split into two or more inactive fragments by directly cleaving the DddA at one or more split sites. Direct cleaving can be carried out by a protease (e.g., trypsin) or other enzyme or chemical reagent. In certain embodiments, such chemical cleavage reactions can be designed to be site-selective (e.g., Elashal and Raj, “Site-selective chemical cleavage of peptide bonds,” Chemical Communications, 2016, Vol. 52, pages 6304-6307, the contents of which are incorporated herein by reference.) In other embodiments, chemical cleavage reactions can be designed to be non-selective and/or occur in a random fashion.

In other embodiments, the two or more inactive DddA fragments can be engineered as separately expressed polypeptides. For instance, for a DddA having one split site, the N-terminal DddA fragment could be engineered from a first nucleotide sequence that encodes the N-terminal DddA fragment (which extends from the N-terminus of the DddA up to and including the residue on the amino-terminal side of the split site). In such an example, the C-terminal DddA fragment could be engineered from a second nucleotide sequence that encodes the C-terminal DddA fragment (which extends from the carboxy-terminus of the split site up to including the natural C-terminus of the DddA protein). The first and second nucleotide sequences could be on the same or different nucleotide molecules (e.g., the same or different expression vectors).

In various embodiments, that N-terminal portion of the DddA may be referred to as “DddA-N half” and the C-terminal portion of the DddA may be referred to as the “DddA-C half.” Reference to the term “half” does not connote the requirement that the DddA-N and DddA-C portions are identically half of the size and/or sequence length of a complete DddA, or that the split site is required to be at the mid point of the complete DddA polypeptide. To the contrary, and as noted above, the split site can be between any pair of residues in the DddA polypeptide, thereby giving rise to half portions which are unequal in size and/or sequence length. In certain embodiments, the split site is within a loop region of the DddA.

Accordingly, in one aspect, the disclosure relates to a pair of fusion proteins useful for making modifications to the sequence of mitochondrial DNA (e.g., mtDNA). The pair of fusion proteins, in some embodiments, can comprise a first fusion protein comprising a first pDNAbp (e.g., a mitoTALE, mitoZFP, or a CRISPR/Cas9) and a first portion or fragment of a DddA, and a second fusion protein comprising a second pDNAbp (e.g., mitoTALE, mitoZFP, or a CRISPR/Cas9) and a second portion or fragment of a DddA, such that the first and the second portions of the DddA reconstitute a DddA upon co-localization in a cell and/or mitochondria. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[pDNAbp]-[DddA half^{A}] and [pDNAbp]-[DddA half^{B}];
[DddA-half^{A}]-[pDNAbp] and [DddA-half^{B}]-[pDNAbp];
[pDNAbp]-[DddA half^{A}] and [DddA-half^{B}]-[pDNAbp]; or
[DddA-half^{A}]-[pDNAbp] and [pDNAbp]-[DddA half^{B}], wherein “A” or “B” can be the N-terminal or C-terminal half of DddA.

In another aspect, the disclosure relates to a pair of fusion proteins useful for making modifications to the sequence of mitochondrial DNA (e.g., mtDNA). The pair of fusion proteins can comprise a first fusion protein comprising a first mitoTALE and a first portion or fragment of a DddA, and a second fusion protein comprising a second mitoTALE and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA and the second portion of the DddA is C-terminal fragment of a Ddda. In other embodiments, the first portion of the DddA is a C-terminal fragment of a DddA and the second portion of the DddA is an N-terminal fragment of a DddA. In this aspect, the structure of the pair of fusion proteins can be, for example:
[mitoTALE]-[DddA half^{A}] and [mitoTALE]-[DddA half^{B}];
[DddA-half^{A}]-[pDNAbp] and [DddA-half^{B}]-[mitoTALE];
[mitoTALE]-[DddA half^{A}] and [DddA-half^{B}]-[mitoTALE]; or
[DddA-half^{A}]-[mitoTALE] and [mitoTALE]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first mitoZFP and a first portion or fragment of a DddA, and a second fusion protein comprising a second mitoZFP and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA and the second portion of the DddA is C-terminal fragment of a Ddda. In other embodiments, the first portion of the DddA is a C-terminal fragment of a DddA and the second portion of the DddA is an N-terminal fragment of a DddA. In this aspect, the structure of the pair of fusion proteins can be, for example:
[mitoZFP]-[DddA half^{A}] and [mitoZFP]-[DddA half^{B}];
[DddA-half^{A}]-[pDNAbp] and [DddA-half^{B}]-[mitoZFP];
[mitoZFP]-[DddA half^{A}] and [DddA-half^{B}]-[mitoZFP]; or
[DddA-half^{A}]-[mitoZFP] and [mitoZFP]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first Cas9 and a first portion or fragment of a DddA, and a second fusion protein comprising a second Cas9 and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA (i.e., “DddA half^{A}” as shown in FIGS. 1A-1E) and the second portion of the DddA is C-terminal fragment of a DddA (i.e., “DddA half^{B}” as shown in FIGS. 1A-1E). In other embodiments, the first portion of the DddA is an C-terminal fragment of a DddA and the second portion of the DddA is an N-terminal fragment of a DddA. In this aspect, the structure of the pair of fusion proteins can be, for example:
[Cas9]-[DddA half^{A}] and [Cas9]-[DddA half^{B}];
[DddA-half^{A}]-[Cas9] and [DddA-half^{B}]-[Cas9];
[Cas9]-[DddA half^{A}] and [DddA-half^{B}]-[Cas9]; or
[DddA-half^{A}]-[Cas9] and [Cas9]-[DddA half^{B}], wherein “A” or “B” can be the N-terminal or C-terminal half of DddA.

In each instance above of “]-[” can be in reference to a linker sequence.

In some embodiments, a first fusion protein comprises, a first mitochondrial transcription activator-like effector (mitoTALE) domain and a first portion of a DNA deaminase effector (DddA). In some embodiments, the first portion of the DddA comprises an N-terminal truncated DddA. In some embodiments, the first mitoTALE is configured to bind a first nucleic acid sequence proximal to a target nucleotide. In some embodiments, the first portion of a DddA is linked to the remainder of the first fusion protein by the C-terminus of the first portion of a DddA.

In one aspect, the present disclosure provides mitochondrial DNA editor fusion proteins for use in editing mitochondrial DNA. As used herein, these mitochondrial DNA editor fusion proteins may be referred to as “mtDNA editors” or “mtDNA editing systems.”

In various embodiments, the mtDNA editors described herein comprise (1) a programmable DNA binding protein (“pDNAbp”) (e.g., a mitoTALE domain, mitoZFP domain, or a CRISPR/Cas9 domain) and a double-stranded DNA deaminase domain, which is capable of carrying out a deamination of a nucleobase at a target site associated with the binding site of the programmable DNA binding protein (pDNAbp).

In some embodiments, the double-stranded DNA deaminase is split into two inactive half portions, with each half portion being fused to a programmable DNA binding protein that binds to a nucleotide sequence either upstream or downstream of a target edit site, and wherein once in the mitochondria, the two half portions (i.e., the N-terminal half and the C-terminal half) reassociate at the target edit site by the co-localization of the programmable DNA binding proteins to binding sites upstream and downstream of the target edit site to be acted on by the DNA deaminase. The reassociation of the two half portions of the double-stranded DNA deaminase restores the deaminase activity at the target edit site. In other embodiments, the double-stranded DNA deaminase can initially be set in an inactive state which can be induced when in the mitochondria. The double-stranded DNA deaminase is preferably delivered initially in an inactive form in order to avoid toxicity inherent with the protein. Any means to regulate the toxic properties of the double-stranded DNA deaminase until such time as the activity is desired to be activated (e.g., in the mitochondria) is contemplated.

In various embodiments, the following exemplary DddA enzymes can be used with the mtDNA base editors described herein, or a sequence (amino acid or nucleotide as the case may be) having at least 70%, 75%, 80%, 85%, 90%, 95%, or 99% sequence identity with any one of the following mitoTALE sequences:

| DddA | |
|---|---|
| Description | DddA amino acid and/or nucleotid sequence |
| DddA | >ATF83755.1 hypothetical protein CO712_00910 [Burkholderia gladioli pv. |
| homolog in | gladioli] |
| Burkholderia | MYEAARVTDPIEHTSALAGFLVGAVLGIALIAAVAFATFTCGFGVALL |
| gladioli | AGMAAGIGAQVLLSLGESIGKMFSSQSGAITLGSPNVYVNGKQAAYA |
| PROTEIN | TLSSVTCSKHNPTPLVAQGSTNIFINGKPAARKDDKITCGAAISDGSHD |
| | TYFHGGIQTCLPIDDEVPPWLRTATDWAFALAGLVGGLGGLLKEAGG |
| | LSHAVMPCAAKFIGGYVLGEAASRYVIGPAINSAIGGMFGNPVDVTT |
| | GRKILPAESETDYVVPSPMPVAIRRFYSSDLDYVGTLGRGWVLPWEL |
| | RLHARDGRLWYTDAQGRESGFPILKPGQAAFSEADQRYLTCTPDGRY |
| | ILHDVGETYYDFGRYEPGSGRIGWVRRIEDQAGQWCQFERDSRGRVR |
| | EIQTCGGLLAVLDYEPEHERLAEVSLVSGDQRRLVVAYGYDENGQM |
| | ASVTDANGAVVRRFTYADGRMTSHSNALGFTSGYTWKVIDGTPRVV |
| | ATHTSEGEAWAFEYDIEGRRTHVRHADGRHAQWRYDAQFQIVEYLD |
| | FDGRRYGLKYNAAGMPVMLTLPGERTVMFEYDDAGRIVAETDPLGR |
| | TTKTRYDGNSMRPVEIILPDGSAWHAEYDRQGRLLVTRDPLDRENRY |
| | EYPEALSALPVAHVDALGGRKTFEWNRLGELVAYTDCSGKTTRNFF |
| | DAFGLPLARENALGHRVSFDLRPTGETRRVTYPDGSSESYEYDAAGL |
| | MIRHIGLGGRMQTLQRNARGQLVEAVDPAGRRTRYHYDAEGRLREL |
| | QQAHARYAFAYSAGGRLVSETRPDGVLRRFEYGEAGDLAALEIVGT |
| | ADDCAPNDRPVRAIRFERDRMGNLCVQHTPTEVTRYERDAGGRLLE |
| | VASVPTAAGLALGIAPDTLTFEYDKAGRLSAEHGANGSVQYTLDALD |
| | NVLKLALPHEQTLQMLRYGSGHVHQIRHGDQVVSDFERDDLHRELT |
| | RTQGPLTERTAYDLLGRKIWQSAGFQPDALARGQGQLWRNYGYDA |
| | AGELVESHDSLRGSTQFSYDPAGYLTQRVNTADRQLESFAWDAAGN |
| | LLDDAQRSSRGYVEGNRLRMWQNLRFDYDAFGNLATKLRGANQRQ |
| | QFTYDGQDRLVAVRTQGARGVVETRFAYDPLGRRIAKTDRTLDVRG |
| | VTLREETKRFVWEGLRLAQEVRDTGVSSYVYSPDAPYMPAARVDAV |
| | KAEALANAAIDKARQATRIYHFHTDVSGAPQEATNEAGDIVWAGQY |
| | SAWGKVAPNQHAPARIDQPLRYAGQYADDSTELHYNTFRFYDPDVG |
| | RFINQDPIGLMGGLNLYQYAPNSIAWTDWWGLAGSYTLGSYQISAPQ |
| | LPAYNGQTVGTFYYVNDAGGLESRTFSSGGPTPYPNYANAGHVEGQ |
| | SALFMRDNGISDGLVFHNNPEGTCGFCVNMTETLLPENSKLTVVPPE |
| | GSIPVKRGATGETRTFTGNSKSPKSPVKGGC [SEQ ID NO: 130] |
| DddA | >CO712_00910 NZ_CP023522.1: 185368-189645 Burkholderia gladioli pv. |
| homolog in | gladioli strain FDAARGOS_389 chromosome 1, complete sequence |
| Burkholderia | GTGTACGAAGCGGCCCGCGTCACGGATCCGATCGAGCACACCAGC |
| gladioli | GCGCTGGCCGGCTTCCTGGTGGGCGCCGTGCTCGGTATCGCCCTGA |
| DNA | TTGCTGCCGTGGCGTTCGCCACGTTCACCTGCGGCTTCGGCGTGGC |
| | ACTGCTGGCCGGCATGGCGGCCGGCATCGGCGCGCAGGTGCTGTT |
| | GTCGTTAGGGGAATCGATCGGGAAGATGTTCAGTTCGCAATCCGG |
| | CGCGATCACGCTCGGCTCGCCGAACGTCTACGTGAACGGCAAGCA |
| | GGCCGCCTACGCCACGCTCAGCAGCGTGACGTGCAGCAAGCACAA |
| | CCCGACGCCGCTCGTCGCGCAGGGCTCCACCAACATCTTCATCAAC |
| | GGCAAGCCGGCCGCGCGCAAGGACGACAAGATCACCTGCGGCGC |
| | GGCCATCTCGGACGGCTCGCACGACACCTACTTCCACGGAGGCAT |
| | CCAGACCTGCCTGCCGATCGACGACGAAGTGCCGCCGTGGCTGCG |
| | CACCGCCACCGACTGGGCGTTCGCGCTGGCCGGGCTGGTGGGCGG |
| | GCTCGGCGGCCTACTCAAGGAAGCGGGCGGGCTGTCGCACGCGGT |
| | GATGCCGTGCGCGGCGAAGTTCATCGGCGGCTACGTGCTCGGCGA |
| | GGCGGCGAGCCGCTACGTGATCGGCCCGGCCATCAACAGCGCGAT |
| | CGGCGGGATGTTCGGCAACCCGGTAGACGTCACCACTGGGCGCAA |
| | GATCCTCCCTGCCGAATCGGAAACCGATTACGTCGTGCCCAGCCC |
| | GATGCCGGTGGCGATCCGGCGCTTCTATTCGAGCGACCTCGATTAC |
| | GTCGGCACGCTTGGGCGCGGCTGGGTGCTGCCGTGGGAGCTGCGC |
| | CTGCACGCGCGTGACGGTCGGCTCTGGTACACCGACGCGCAGGGG |
| | CGCGAGAGCGGCTTCCCGATCCTGAAACCGGGCCAGGCCGCGTTC |
| | AGCGAGGCCGATCAGCGCTATCTGACCTGCACGCCGGATGGCCGC |
| | TACATCCTCCACGACGTCGGCGAAACCTATTACGACTTCGGCCGCT |
| | ACGAGCCGGGCTCGGGCCGCATCGGCTGGGTGCGCCGGATCGAGG |
| | ATCAGGCCGGCCAGTGGTGCCAGTTCGAGCGCGACAGCCGTGGCC |
| | GCGTGCGTGAAATCCAGACCTGCGGCGGCTTGCTGGCCGTGCTCG |
| | ATTACGAGCCGGAGCACGAGCGGCTCGCCGAGGTGTCGCTCGTCA |
| | GCGGCGATCAGCGCCGCCTCGTCGTGGCCTACGGCTACGACGAAA |
| | ACGGCCAGATGGCCTCCGTGACCGACGCGAACGGCGCGGTGGTGC |
| | GCCGCTTCACCTATGCCGACGGGCGCATGACGAGCCATTCGAACG |
| | CGCTCGGTTTCACGTCGGGCTATACGTGGAAGGTCATCGACGGCA |
| | CGCCGCGAGTGGTCGCCACCCACACCAGCGAGGGCGAGGCCTGGG |
| | CGTTCGAGTACGACATCGAAGGCCGCCGCACCCATGTGCGGCATG |
| | CCGACGGCCGCCACGCGCAATGGCGCTACGACGCGCAATTCCAGA |
| | TCGTCGAGTACCTCGATTTCGACGGCCGTCGCTACGGGCTCAAGTA |
| | CAACGCTGCCGGCATGCCCGTGATGCTGACGCTGCCCGGCGAACG |
| | AACCGTGATGTTCGAGTACGACGACGCCGGCCGCATCGTCGCCGA |
| | AACCGATCCCCTCGGCCGCACCACGAAAACGCGCTACGACGGCAA |
| | CAGCATGCGGCCCGTCGAGATCATCTTGCCCGACGGCAGCGCCTG |
| | GCACGCCGAATACGACCGGCAGGGCCGGCTGCTCGTCACCCGTGA |
| | TCCGCTCGACCGGGAGAATCGCTACGAATATCCGGAGGCACTGAG |
| | CGCGCTCCCGGTGGCGCATGTCGATGCGCTGGGCGGGCGCAAGAC |
| | GTTCGAGTGGAACCGGCTCGGCGAGCTGGTGGCCTACACCGATTG |
| | CTCGGGCAAGACCACGCGCAATTTTTTCGATGCATTCGGCCTGCCG |
| | CTCGCGCGCGAGAACGCGCTCGGGCACCGCGTGTCGTTCGATCTG |
| | CGCCCGACCGGCGAGACGCGCCGCGTCACCTATCCCGACGGCAGT |
| | TCCGAAAGCTACGAATACGACGCCGCCGGGCTGATGATCCGGCAC |
| | ATCGGGCTGGGCGGCCGGATGCAGACGTTGCAGCGCAATGCGCGC |
| | GGGCAACTCGTCGAGGCGGTCGATCCGGCCGGGCGGCGAACCCGC |
| | TACCACTACGACGCCGAAGGGCGGCTGCGCGAGCTGCAACAGGCC |
| | CACGCGCGCTACGCATTCGCGTACAGCGCAGGCGGGCGGCTTGTC |
| | AGCGAAACGCGGCCCGACGGCGTGCTGCGCCGCTTCGAATACGGC |
| | GAGGCCGGCGATCTGGCGGCGCTCGAGATCGTCGGAACGGCCGAT |
| | GATTGCGCTCCAAACGATCGCCCGGTTCGCGCGATCCGCTTCGAGC |
| | GCGACCGGATGGGTAACCTGTGCGTGCAGCACACGCCTACCGAGG |
| | TGACGCGCTACGAGCGCGACGCCGGCGGCCGCCTGCTCGAAGTCG |
| | CGAGCGTGCCGACCGCGGCCGGACTGGCGCTCGGCATCGCGCCCG |
| | ACACGCTGACCTTCGAATACGACAAGGCCGGGCGGCTGAGCGCCG |
| | AACACGGCGCGAACGGCAGCGTCCAGTACACGCTCGACGCGCTCG |
| | ACAACGTGTTGAAGCTCGCCTTGCCGCACGAACAGACGCTGCAGA |
| | TGCTGCGCTACGGCTCGGGGCACGTGCACCAGATTCGCCACGGCG |
| | ACCAGGTCGTCAGCGATTTCGAGCGCGACGACCTGCATCGCGAGT |
| | TGACGCGCACGCAGGGCCCCCTGACCGAGCGGACCGCCTACGACC |
| | TGCTGGGCCGCAAGATCTGGCAATCAGCCGGCTTCCAGCCCGACG |
| | CGCTTGCGCGTGGGCAGGGCCAGCTGTGGCGCAACTACGGCTACG |
| | ACGCCGCCGGGGAACTGGTCGAGAGCCACGACAGCCTGCGCGGCA |
| | GCACGCAGTTCAGCTACGATCCGGCCGGCTATCTGACGCAGCGCG |
| | TGAACACCGCCGACCGGCAGCTCGAATCGTTCGCCTGGGACGCCG |
| | CCGGCAACCTGCTCGACGATGCGCAACGCAGCAGCCGCGGCTATG |
| | TCGAGGGCAACCGGCTGCGCATGTGGCAGAACCTGCGCTTCGACT |
| | ACGACGCGTTCGGCAATCTCGCGACCAAGCTGCGCGGCGCGAATC |
| | AGCGCCAGCAGTTCACGTACGATGGGCAGGATCGGCTCGTGGCCG |
| | TGCGCACGCAGGGCGCGCGCGGCGTGGTGGAGACGCGTTTCGCCT |
| | ACGATCCGCTCGGGCGGCGCATCGCCAAGACCGATAGGACACTCG |
| | ACGTGCGCGGCGTAACGCTGCGCGAGGAAACGAAGCGGTTCGTAT |
| | GGGAAGGGCTGCGGCTCGCGCAGGAGGTGCGCGACACCGGCGTG |
| | AGCAGCTACGTGTACAGCCCGGATGCGCCTTACATGCCCGCGGCG |
| | CGGGTCGATGCGGTGAAAGCCGAAGCGCTCGCAAACGCCGCGATC |
| | GACAAGGCCAGACAGGCGACGCGGATCTATCACTTTCATACCGAT |
| | GTGTCGGGCGCACCGCAAGAAGCGACGAACGAGGCCGGCGACAT |
| | TGTTTGGGCCGGCCAATACTCAGCCTGGGGCAAGGTGGCGCCGAA |
| | CCAGCATGCCCCAGCCCGGATCGATCAGCCGCTCCGCTACGCCGG |
| | ACAATATGCCGATGACAGTACCGAGCTGCACTACAACACGTTTCG |
| | TTTCTACGATCCGGATGTCGGCCGGTTTATCAATCAGGATCCAATC |
| | GGGTTGATGGGGGGGCTGAATCTTTACCAATATGCACCCAACTCA |
| | ATCGCGTGGACCGACTGGTGGGGGCTGGCCGGCAGCTATACGCTC |
| | GGTTCCTATCAAATTTCTGCTCCTCAACTTCCCGCCTACAATGGGC |
| | AGACTGTTGGGACCTTCTACTATGTAAACGACGCGGGCGGGCTCG |
| | AATCGAGGACATTCTCTTCTGGAGGGCCGACCCCTTATCCAAATTA |
| | TGCCAATGCCGGGCACGTGGAAGGCCAGTCCGCACTGTTCATGAG |
| | GGATAACGGAATTTCAGACGGACTGGTTTTCCACAACAACCCTGA |
| | GGGTACTTGCGGATTCTGCGTCAATATGACCGAAACGCTTTTGCCT |
| | GAAAATTCCAAACTTACCGTCGTTCCGCCCGAGGGCTCGATTCCGG |
| | TCAAGCGGGGCGCGACGGGCGAAACGAGAACATTTACAGGGAAC |
| | AGCAAGTCTCCGAAGTCCCCTGTCAAAGGAGGATGTTGA (SEQ ID |
| | NO: 131) |
| DddA | >AJY63123.1 RHS repeat-associated core domain protein [Burkholderia |
| homolog in | glumae LMG 2196 = ATCC 33617] |
| Burkholderia | MYEAARVTDPIEHTSALTGFLVGAVLGIALIAAVAFATFTCGFGVALL |
| glumae LMG | AGMAAGIGAQVLLSLGESIGKMFSSQSGAITLGSPNVYVNGKPTAYA |
| 2196 | MLSSVTCSKHNPTPLVAQGSTNIFINGKPAARKDDKITCGATISDGSH |
| PROTEIN | DTYFHGGTQTCLPIDDEVPPWLRTATDWAFALAGLVGGLGGLLKEA |
| | GGLSRAVMPCAAKFIGGYVLGEAASRYVVGPAINSAIGGMFGNPVDV |
| | TTGRKILLAESETDYVVPSPMPVAIRRFYSSDLDYVGTLGRGWVLPW |
| | ELRLHARDGRLWYTDAQGRESGFPMLQPGHAAFSEADQRYLTCTPD |
| | GRYILHDLGETYYDFGHYEPGSGRIGWVRRIEDQAGQWCQFERDSRG |
| | RVREIQTCGGLLAVLDYEPEHGRLAGVSLVSGDQRRLVVAYGYDEH |
| | GQMASVTDANGALVRRFTYADGRMTSHSNALGFTSGYTWQAVGGA |
| | PRVVATHTSEGEAWAFEYDIEGRRTHVRHADGRHAQWRYDAQFQIV |
| | EYLDFDGRRYGLKYNDAGMPVMLTLPGERTVTFEYDDAGRIVAETD |
| | PLGRTTKTRYDGNSRRPVEIIAPDGSAWHAEYDRQGRLLATRDPLDR |
| | ENRYEYPKALSALPIAHVDALGGRKTFEWNRLGELVAYTDCSGKTTR |
| | NFYDAFGLPLARENALGHRVTFDLRPTGEARRVTYPDGSTESYEYDA |
| | AGLMIRHVGLGGRTQIALRNARGQIVEAVDPAGRRTCYRYDAEGRL |
| | RELQQGHARYAFTYSAGGRLTSETRPDGVRRRFEYGEAGDLAALDIV |
| | GAADDATANDRPVRTIRFERDRMGNLCAQHTPTEVTRYTRDTGGRL |
| | LEVACVPTAAGLALGIAPDTLTFEYDKAGRLSAEHGANGSVRYTLDA |
| | LDNVMKLALPHEQTLQMLRYGSGHVHQIRCGDQVVSDFERDDLHRE |
| | LTRTQGRLTERTAYDLLGRKIWQSAGFQPDALARGQGQVWRNYGY |
| | DAAGELAESHDSLRGSTQFSYDPAGYLTQRVNTADRQLESFAWDAA |
| | GNLLDDAQRRSRGYVEGNRLRMWQNLRFEYDPFGNLATKLRGANQ |
| | RQQFTYDGQDRLVAVRTQDARGVVETRFAYDPLGRRIAKTDIVRDA |
| | RGVALREETKRFVWEGLRLAQEVRDTGVSSYVYSPDAPYTPAARVD |
| | AVLAEAMAAAAIEQARQATRIYHFHTDVSGAPQEATNEAGDIVWAG |
| | QYSAWGKVAPNQHAPARIDQPLRYAGQYADDSTELHYNTFRFYDPD |
| | VGRFINQDPIGLMGGLNLYQYAPNSIAWTDWWGLAGSYTLGSYQISA |
| | PQLPAYNGQTVGTFYYVNGAGGLESRTFSSGGPTPYPNYANAGHVE |
| | GQSALFMRDNGISDGLVFHNNPEGTCGFCVNMTETLLPENSKLTVVP |
| | PEGAIPVKRGATGETRTFTGNSKSPKSPVKGEC [SEQ ID NO: 132] |
| DddA | >KS03_3390 CP009434.1: 65330-69607 Burkholderia glumae LMG 2196 = |
| homolog in | ATCC 33617 chromosome II, complete sequence |
| Burkholderia | GTGTACGAAGCGGCCCGCGTCACCGACCCGATCGAACACACCAGC |
| | GCGCTGACCGGCTTTCTGGTGGGCGCCGTGCTCGGCATTGCCCTGA |
| glumae LMG | TCGCCGCGGTGGCGTTCGCCACCTTCACCTGCGGCTTCGGCGTGGC |
| 2196 | GCTGCTGGCCGGCATGGCCGCCGGCATCGGCGCGCAGGTGCTGTT |
| DNA | GTCGTTAGGAGAATCGATCGGGAAGATGTTCAGTTCGCAATCCGG |
| | CGCGATCACGCTCGGCTCGCCGAACGTCTATGTGAACGGCAAGCC |
| | GACCGCCTACGCCATGCTCAGCAGCGTGACGTGCAGCAAGCACAA |
| | CCCGACGCCGCTCGTCGCGCAGGGGTCCACCAACATCTTCATCAA |
| | CGGCAAGCCGGCCGCCCGCAAGGACGACAAGATCACCTGCGGCGC |
| | GACCATCTCCGACGGCTCGCACGACACCTATTTCCACGGCGGCAC |
| | CCAGACCTGCCTGCCGATCGACGACGAAGTGCCGCCGTGGCTGCG |
| | CACCGCCACCGACTGGGCGTTCGCGCTGGCCGGGCTGGTGGGCGG |
| | GCTCGGCGGCCTGCTCAAGGAAGCGGGGGGCTGTCGCGCGCGGT |
| | GATGCCGTGCGCGGCGAAGTTCATCGGCGGCTACGTGCTCGGCGA |
| | GGCGGCGAGCCGCTACGTGGTCGGCCCGGCCATCAACAGCGCGAT |
| | CGGCGGGATGTTCGGCAACCCGGTGGACGTCACCACCGGGCGCAA |
| | GATCCTGCTGGCGGAATCGGAAACCGATTACGTGGTGCCCAGCCC |
| | GATGCCGGTGGCGATCCGGCGCTTCTATTCGAGCGACCTCGACTAC |
| | GTCGGCACGCTCGGGCGCGGCTGGGTGCTGCCGTGGGAACTGCGG |
| | CTGCACGCGCGCGACGGGCGGCTCTGGTACACCGACGCGCAGGGG |
| | CGCGAGAGCGGCTTCCCGATGCTCCAGCCGGGCCATGCCGCGTTC |
| | AGCGAGGCCGACCAGCGCTATCTGACCTGCACCCCGGATGGCCGC |
| | TACATCCTGCACGACCTCGGCGAAACCTATTACGACTTCGGCCACT |
| | ACGAGCCGGGCTCGGGCCGCATCGGCTGGGTGCGCCGCATCGAGG |
| | ATCAGGCCGGCCAGTGGTGCCAGTTCGAGCGCGACAGCCGCGGCC |
| | GCGTGCGCGAAATCCAGACCTGCGGCGGCTTGCTGGCCGTGCTCG |
| | ATTACGAGCCGGAACACGGGCGGCTCGCCGGGGTGTCGCTCGTCA |
| | GCGGGGATCAGCGCCGCCTCGTGGTGGCTTACGGCTATGACGAGC |
| | ACGGCCAGATGGCGTCCGTGACCGATGCGAACGGCGCGCTGGTGC |
| | GCCGCTTCACCTATGCCGACGGGCGCATGACGAGCCATTCGAACG |
| | CGCTCGGCTTCACGTCGGGCTATACGTGGCAAGCCGTCGGCGGCG |
| | CGCCGCGGGTGGTTGCCACCCACACCAGCGAGGGCGAGGCCTGGG |
| | CCTTCGAGTACGACATTGAAGGACGCCGCACCCACGTGCGTCACG |
| | CCGACGGCCGCCACGCGCAATGGCGCTACGACGCGCAATTCCAGA |
| | TCGTCGAGTACCTCGATTTCGACGGCCGGCGCTACGGGCTCAAGT |
| | ACAACGACGCCGGCATGCCCGTGATGCTGACGCTGCCCGGCGAAC |
| | GGACCGTGACGTTCGAGTACGACGATGCCGGCCGCATCGTCGCCG |
| | AAACCGATCCACTCGGCCGCACCACGAAAACGCGCTACGACGGCA |
| | ACAGCAGGCGGCCCGTCGAGATCATCGCGCCCGACGGCAGCGCCT |
| | GGCACGCCGAATACGACCGGCAAGGCCGGCTGCTCGCCACCCGCG |
| | ATCCGCTCGACCGGGAAAACCGCTACGAATACCCGAAGGCGCTCA |
| | GCGCGCTGCCGATCGCGCACGTCGATGCGCTGGGCGGGCGCAAGA |
| | CGTTCGAGTGGAACCGGCTCGGCGAGCTGGTGGCCTATACCGATT |
| | GCTCGGGCAAGACCACACGCAATTTTTACGACGCATTCGGTCTGCC |
| | GCTCGCGCGCGAGAACGCGCTCGGCCACCGCGTGACGTTCGACCT |
| | GCGCCCGACCGGCGAGGCGCGGCGCGTCACCTATCCCGACGGCAG |
| | TACAGAAAGCTACGAATACGACGCCGCCGGGCTGATGATCCGGCA |
| | CGTCGGGCTGGGCGGCCGGACGCAGATTGCGCTGCGCAACGCGCG |
| | TGGGCAGATCGTGGAGGCGGTCGATCCGGCCGGACGGCGCACCTG |
| | CTACCGCTACGACGCCGAGGGGCGGCTGCGCGAGCTGCAACAGGG |
| | GCACGCGCGTTACGCGTTCACCTACAGCGCGGGCGGGCGGCTCAC |
| | CAGCGAAACCCGGCCCGACGGCGTGCGGCGCCGCTTCGAATACGG |
| | CGAGGCCGGCGATCTGGCGGCGCTCGACATCGTCGGCGCGGCCGA |
| | CGACGCCACGGCGAACGATCGTCCGGTTCGCACCATCCGCTTCGA |
| | GCGCGACCGCATGGGCAATCTGTGCGCGCAGCACACGCCCACCGA |
| | GGTGACGCGCTACACGCGCGACACCGGCGGCCGCCTGCTCGAAGT |
| | CGCATGCGTGCCGACCGCGGCCGGGCTGGCGCTCGGCATCGCGCC |
| | CGACACGCTGACCTTCGAATACGACAAGGCCGGGCGGCTGAGTGC |
| | CGAACACGGCGCGAACGGCAGCGTCCGATACACGCTCGACGCGCT |
| | CGACAACGTGATGAAGCTCGCCCTGCCGCACGAGCAGACGCTGCA |
| | GATGCTGCGCTACGGCTCGGGGCACGTGCATCAGATCCGCTGCGG |
| | CGACCAGGTGGTCAGCGATTTCGAGCGCGACGACCTGCATCGCGA |
| | GCTGACGCGCACTCAGGGCCGCCTGACCGAGCGTACCGCCTACGA |
| | CCTGCTGGGCCGCAAGATCTGGCAATCGGCCGGCTTCCAGCCCGA |
| | CGCGCTTGCGCGCGGGCAGGGCCAGGTGTGGCGCAACTACGGCTA |
| | CGACGCCGCCGGCGAACTGGCCGAGAGCCACGATAGCCTGCGCGG |
| | CAGCACGCAGTTCAGCTACGATCCGGCCGGCTATCTGACGCAGCG |
| | CGTCAATACCGCCGACCGGCAGCTCGAATCGTTCGCCTGGGATGC |
| | CGCCGGCAACCTGCTCGACGATGCGCAGCGCCGCAGCCGCGGTTA |
| | TGTCGAGGGCAACCGGCTGCGCATGTGGCAGAACCTGCGCTTCGA |
| | ATACGACCCGTTCGGCAATCTCGCGACCAAGCTGCGCGGCGCGAA |
| | CCAGCGCCAGCAGTTCACTTACGACGGGCAGGATCGGCTCGTGGC |
| | GGTGCGCACGCAGGACGCGCGCGGCGTGGTGGAGACGCGTTTCGC |
| | CTACGATCCGCTGGGGCGGCGCATCGCCAAGACGGATATTGTGCG |
| | CGACGCGCGCGGCGTAGCGCTGCGCGAGGAAACGAAGCGGTTCGT |
| | GTGGGAGGGGCTGCGGCTCGCGCAGGAGGTGCGCGACACGGGCG |
| | TGAGCAGCTACGTGTACAGCCCGGACGCGCCCTATACGCCCGCGG |
| | CGCGCGTGGATGCCGTGCTGGCCGAGGCCATGGCCGCCGCTGCCA |
| | TCGAGCAGGCCAGACAGGCGACGCGGATCTATCACTTTCATACCG |
| | ATGTGTCGGGCGCACCGCAAGAAGCGACGAACGAGGCTGGCGAC |
| | ATTGTTTGGGCCGGCCAATACTCAGCCTGGGGCAAGGTGGCGCCG |
| | AACCAGCATGCCCCCGCCCGGATCGATCAGCCGCTCCGCTACGCC |
| | GGACAATATGCCGACGACAGTACCGAGCTGCACTACAACACGTTT |
| | CGTTTCTACGATCCGGACGTCGGCCGGTTTATCAATCAGGATCCAA |
| | TCGGGTTGATGGGGGGGCTGAATCTTTACCAATATGCACCCAACTC |
| | GATCGCATGGACCGACTGGTGGGGGCTGGCCGGCAGCTATACGCT |
| | CGGTTCCTATCAAATTTCTGCGCCTCAACTTCCGGCCTACAATGGA |
| | CAGACTGTTGGGACCTTCTACTACGTGAACGGCGCGGGCGGGCTC |
| | GAATCGAGGACATTCTCTTCCGGAGGGCCGACCCCTTATCCAAATT |
| | ATGCCAATGCCGGGCACGTGGAGGGCCAGTCCGCGCTGTTCATGA |
| | GGGATAACGGAATTTCAGACGGACTGGTTTTCCACAACAACCCTG |
| | AGGGCACTTGCGGATTCTGCGTTAATATGACCGAAACGCTTTTGCC |
| | TGAAAATTCCAAACTTACCGTCGTTCCGCCCGAGGGCGCGATCCC |
| | GGTCAAGCGGGGCGCGACGGGCGAAACGAGAACATTTACGGGGA |
| | ACAGCAAGTCTCCGAAGTCCCCTGTCAAAGGAGAATGTTGA [SEQ |
| | ID NO: 133] |
| DddA | >ACR30728.1 Rhs family protein [Burkholderia glumae BGR1] |
| homolog in | MYEAARVTDPIEHTSALTGFLVGAVLGIALIAAVAFATFTCGFGVALL |
| Burkholderia | AGMAAGIGAQVLLSLGESIGKMFSSQSGAITLGSPNVYVNGKPTAYA |
| glumae BGR1 | MLSSVTCSKHNPTPLVAQGSTNIFINGKPAARKDDKITCGATISDGSH |
| PROTEIN | DTYFHGGTQTCLPIDDEVPPWLRTATDWAFALAGLVGGLGGLLKEA |
| | GGLSRAVMPCAAKFIGGYVLGEAASRYVVGPAINSAIGGMFGNPVDV |
| | TTGRKILLAESETDYVVPSPMPVAIRRFYSSDLDYVGTLGRGWVLPW |
| | ELRLHARDGRLWYTDAQGRESGFPMLQPGHAAFSEADQRYLTCTPD |
| | GRYILHDLGETYYDFGHYEPGSGRIGWVRRIEDQAGQWCQFERDSRG |
| | RVREIQTCGGLLAVLDYEPEHGRLAGVSLVSGDQRRLVVAYGYDEH |
| | GQMASVTDANGALVRRFTYADGRMTSHSNALGFTSGYTWQAVGGA |
| | PRVVATHTSEGEAWAFEYDIEGRRTHVRHADGRHAQWRYDAQFQIV |
| | EYLDFDGRRYGLKYNDAGMPVMLTLPGERTVTFEYDDAGRIVAETD |
| | PLGRTTKTRYDGNSRRPVEIIAPDGSAWHAEYDRQGRLLATRDPLDR |
| | ENRYEYPKALSALPIAHVDALGGRKTFEWNRLGELVAYTDCSGKTTR |
| | NFYDAFGLPLARENALGHRVTFDLRPTGEARRVTYPDGSTESYEYDA |
| | AGLMIRHVGLGGRTQIALRNARGQIVEAVDPAGRRTCYRYDAEGRL |
| | RELQQGHARYAFTYSAGGRLTSETRPDGVRRRFEYGEAGDLAALDIV |
| | GAADDATANDRPVRTIRFERDRMGNLCAQHTPTEVTRYTRDTGGRL |
| | LEVACVPTAAGLALGIAPDTLTFEYDKAGRLSAEHGANGSVRYTLDA |
| | LDNVMKLALPHEQTLQMLRYGSGHVHQIRCGDQVVSDFERDDLHRE |
| | LTRTQGRLTERTAYDLLGRKIWQSAGFQPDALARGQGQVWRNYGY |
| | DAAGELAESHDSLRGSTQFSYDPAGYLTQRVNTADRQLESFAWDAA |
| | GNLLDDAQRRSRGYVEGNRLRMWQNLRFEYDPFGNLATKLRGANQ |
| | RQQFTYDGQDRLVAVRTQDARGVVETRFAYDPLGRRIAKTDIVRDA |
| | RGVALREETKRFVWEGLRLAQEVRDTGVSSYVYSPDAPYTPAARVD |
| | AVLAEAMAAAAIEQARQATRIYHFHTDVSGAPQEATNEAGDIVWAG |
| | QYSAWGKVAPNQHAPARIDQPLRYAGQYADDSTELHYNTFRFYDPD |
| | VGRFINQDPIGLMGGLNLYQYAPNSIAWTDWWGLAGSYTLGSYQISA |
| | PQLPAYNGQTVGTFYYVNGAGGLESRTFSSGGPTPYPNYANAGHVE |
| | GQSALFMRDNGISDGLVFHNNPEGTCGFCVNMTETLLPENSKLTVVP |
| | PEGAIPVKRGATGETRTFTGNSKSPKSPVKGEC [SEQ ID NO: 134] |
| DddA | >bglu_2g02600 NC_012721.2: 303868-308145 Burkholderia glumae BGR1 |
| homolog in | chromosome 2, complete sequence |
| Burkholderia | GTGTACGAAGCGGCCCGCGTCACCGACCCGATCGAACACACCAGC |
| glumae BGR1 | GCGCTGACCGGCTTTCTGGTGGGCGCCGTGCTCGGCATTGCCCTGA |
| DNA | TCGCCGCGGTGGCGTTCGCCACCTTCACCTGCGGCTTCGGCGTGGC |
| | GCTGCTGGCCGGCATGGCCGCCGGCATCGGCGCGCAGGTGCTGTT |
| | GTCGTTAGGAGAATCGATCGGGAAGATGTTCAGTTCGCAATCCGG |
| | CGCGATCACGCTCGGCTCGCCGAACGTCTATGTGAACGGCAAGCC |
| | GACCGCCTACGCCATGCTCAGCAGCGTGACGTGCAGCAAGCACAA |
| | CCCGACGCCGCTCGTCGCGCAGGGGTCCACCAACATCTTCATCAA |
| | CGGCAAGCCGGCCGCCCGCAAGGACGACAAGATCACCTGCGGCGC |
| | GACCATCTCCGACGGCTCGCACGACACCTATTTCCACGGCGGCAC |
| | CCAGACCTGCCTGCCGATCGACGACGAAGTGCCGCCGTGGCTGCG |
| | CACCGCCACCGACTGGGCGTTCGCGCTGGCCGGGCTGGTGGGCGG |
| | GCTCGGCGGCCTGCTCAAGGAAGCGGGCGGGCTGTCGCGCGCGGT |
| | GATGCCGTGCGCGGCGAAGTTCATCGGCGGCTACGTGCTCGGCGA |
| | GGCGGCGAGCCGCTACGTGGTCGGCCCGGCCATCAACAGCGCGAT |
| | CGGCGGGATGTTCGGCAACCCGGTGGACGTCACCACCGGGCGCAA |
| | GATCCTGCTGGCGGAATCGGAAACCGATTACGTGGTGCCCAGCCC |
| | GATGCCGGTGGCGATCCGGCGCTTCTATTCGAGCGACCTCGACTAC |
| | GTCGGCACGCTCGGGCGCGGCTGGGTGCTGCCGTGGGAACTGCGG |
| | CTGCACGCGCGCGACGGGCGGCTCTGGTACACCGACGCGCAGGGG |
| | CGCGAGAGCGGCTTCCCGATGCTCCAGCCGGGCCATGCCGCGTTC |
| | AGCGAGGCCGACCAGCGCTATCTGACCTGCACCCCGGATGGCCGC |
| | TACATCCTGCACGACCTCGGCGAAACCTATTACGACTTCGGCCACT |
| | ACGAGCCGGGCTCGGGCCGCATCGGCTGGGTGCGCCGCATCGAGG |
| | ATCAGGCCGGCCAGTGGTGCCAGTTCGAGCGCGACAGCCGCGGCC |
| | GCGTGCGCGAAATCCAGACCTGCGGCGGCTTGCTGGCCGTGCTCG |
| | ATTACGAGCCGGAACACGGGCGGCTCGCCGGGGTGTCGCTCGTCA |
| | GCGGGGATCAGCGCCGCCTCGTGGTGGCTTACGGCTATGACGAGC |
| | ACGGCCAGATGGCGTCCGTGACCGATGCGAACGGCGCGCTGGTGC |
| | GCCGCTTCACCTATGCCGACGGGCGCATGACGAGCCATTCGAACG |
| | CGCTCGGCTTCACGTCGGGCTATACGTGGCAAGCCGTCGGCGGCG |
| | CGCCGCGGGTGGTTGCCACCCACACCAGCGAGGGCGAGGCCTGGG |
| | CCTTCGAGTACGACATTGAAGGACGCCGCACCCACGTGCGTCACG |
| | CCGACGGCCGCCACGCGCAATGGCGCTACGACGCGCAATTCCAGA |
| | TCGTCGAGTACCTCGATTTCGACGGCCGGCGCTACGGGCTCAAGT |
| | ACAACGACGCCGGCATGCCCGTGATGCTGACGCTGCCCGGCGAAC |
| | GGACCGTGACGTTCGAGTACGACGATGCCGGCCGCATCGTCGCCG |
| | AAACCGATCCACTCGGCCGCACCACGAAAACGCGCTACGACGGCA |
| | ACAGCAGGCGGCCCGTCGAGATCATCGCGCCCGACGGCAGCGCCT |
| | GGCACGCCGAATACGACCGGCAAGGCCGGCTGCTCGCCACCCGCG |
| | ATCCGCTCGACCGGGAAAACCGCTACGAATACCCGAAGGCGCTCA |
| | GCGCGCTGCCGATCGCGCACGTCGATGCGCTGGGCGGGCGCAAGA |
| | CGTTCGAGTGGAACCGGCTCGGCGAGCTGGTGGCCTATACCGATT |
| | GCTCGGGCAAGACCACACGCAATTTTTACGACGCATTCGGTCTGCC |
| | GCTCGCGCGCGAGAACGCGCTCGGCCACCGCGTGACGTTCGACCT |
| | GCGCCCGACCGGCGAGGCGCGGCGCGTCACCTATCCCGACGGCAG |
| | TACAGAAAGCTACGAATACGACGCCGCCGGGCTGATGATCCGGCA |
| | CGTCGGGCTGGGCGGCCGGACGCAGATTGCGCTGCGCAACGCGCG |
| | TGGGCAGATCGTGGAGGCGGTCGATCCGGCCGGACGGCGCACCTG |
| | CTACCGCTACGACGCCGAGGGGCGGCTGCGCGAGCTGCAACAGGG |
| | GCACGCGCGTTACGCGTTCACCTACAGCGCGGGCGGGCGGCTCAC |
| | CAGCGAAACCCGGCCCGACGGCGTGCGGCGCCGCTTCGAATACGG |
| | CGAGGCCGGCGATCTGGCGGCGCTCGACATCGTCGGCGCGGCCGA |
| | CGACGCCACGGCGAACGATCGTCCGGTTCGCACCATCCGCTTCGA |
| | GCGCGACCGCATGGGCAATCTGTGCGCGCAGCACACGCCCACCGA |
| | GGTGACGCGCTACACGCGCGACACCGGCGGCCGCCTGCTCGAAGT |
| | CGCATGCGTGCCGACCGCGGCCGGGCTGGCGCTCGGCATCGCGCC |
| | CGACACGCTGACCTTCGAATACGACAAGGCCGGGCGGCTGAGTGC |
| | CGAACACGGCGCGAACGGCAGCGTCCGATACACGCTCGACGCGCT |
| | CGACAACGTGATGAAGCTCGCCCTGCCGCACGAGCAGACGCTGCA |
| | GATGCTGCGCTACGGCTCGGGGCACGTGCATCAGATCCGCTGCGG |
| | CGACCAGGTGGTCAGCGATTTCGAGCGCGACGACCTGCATCGCGA |
| | GCTGACGCGCACTCAGGGCCGCCTGACCGAGCGTACCGCCTACGA |
| | CCTGCTGGGCCGCAAGATCTGGCAATCGGCCGGCTTCCAGCCCGA |
| | CGCGCTTGCGCGCGGGCAGGGCCAGGTGTGGCGCAACTACGGCTA |
| | CGACGCCGCCGGCGAACTGGCCGAGAGCCACGATAGCCTGCGCGG |
| | CAGCACGCAGTTCAGCTACGATCCGGCCGGCTATCTGACGCAGCG |
| | CGTCAATACCGCCGACCGGCAGCTCGAATCGTTCGCCTGGGATGC |
| | CGCCGGCAACCTGCTCGACGATGCGCAGCGCCGCAGCCGCGGTTA |
| | TGTCGAGGGCAACCGGCTGCGCATGTGGCAGAACCTGCGCTTCGA |
| | ATACGACCCGTTCGGCAATCTCGCGACCAAGCTGCGCGGCGCGAA |
| | CCAGCGCCAGCAGTTCACTTACGACGGGCAGGATCGGCTCGTGGC |
| | GGTGCGCACGCAGGACGCGCGCGGCGTGGTGGAGACGCGTTTCGC |
| | CTACGATCCGCTGGGGCGGCGCATCGCCAAGACGGATATTGTGCG |
| | CGACGCGCGCGGCGTAGCGCTGCGCGAGGAAACGAAGCGGTTCGT |
| | GTGGGAGGGGCTGCGGCTCGCGCAGGAGGTGCGCGACACGGGCG |
| | TGAGCAGCTACGTGTACAGCCCGGACGCGCCCTATACGCCCGCGG |
| | CGCGCGTGGATGCCGTGCTGGCCGAGGCCATGGCCGCCGCTGCCA |
| | TCGAGCAGGCCAGACAGGCGACGCGGATCTATCACTTTCATACCG |
| | ATGTGTCGGGCGCACCGCAAGAAGCGACGAACGAGGCTGGCGAC |
| | ATTGTTTGGGCCGGCCAATACTCAGCCTGGGGCAAGGTGGCGCCG |
| | AACCAGCATGCCCCCGCCCGGATCGATCAGCCGCTCCGCTACGCC |
| | GGACAATATGCCGACGACAGTACCGAGCTGCACTACAACACGTTT |
| | CGTTTCTACGATCCGGACGTCGGCCGGTTTATCAATCAGGATCCAA |
| | TCGGGTTGATGGGGGGGCTGAATCTTTACCAATATGCACCCAACTC |
| | GATCGCATGGACCGACTGGTGGGGGCTGGCCGGCAGCTATACGCT |
| | CGGTTCCTATCAAATTTCTGCGCCTCAACTTCCGGCCTACAATGGA |
| | CAGACTGTTGGGACCTTCTACTACGTGAACGGCGCGGGCGGGCTC |
| | GAATCGAGGACATTCTCTTCCGGAGGGCCGACCCCTTATCCAAATT |
| | ATGCCAATGCCGGGCACGTGGAGGGCCAGTCCGCGCTGTTCATGA |
| | GGGATAACGGAATTTCAGACGGACTGGTTTTCCACAACAACCCTG |
| | AGGGCACTTGCGGATTCTGCGTTAATATGACCGAAACGCTTTTGCC |
| | TGAAAATTCCAAACTTACCGTCGTTCCGCCCGAGGGCGCGATCCC |
| | GGTCAAGCGGGGCGCGACGGGCGAAACGAGAACATTTACGGGGA |
| | ACAGCAAGTCTCCGAAGTCCCCTGTCAAAGGAGAATGTTGA [SEQ |
| | ID NO: 135] |
| DddA | >AOT60363.1 tRNA nuclease WapA precursor [Streptomyces |
| homolog in | rubrolavendulae] |
| Streptomyces | MSSSDAGRAFGVPENVLARFTRYPGGARRRAGRTARARRLGIVLSAV |
| rubrolavendulae | LSATLLPAEAWAIAPPAPRTGPTLDALQQEEEVDPDPAAMEELDDWD |
| PROTEIN | GGPVEPPADYTPTEVTPPTGGTAPVPLDSAGEELVPAGTLPVRIGQAS |
| | PTEEDPAPPAPSGTWDVTVEPRATTEAAAVDGAIIKLTPPASGSTPVD |
| | VELDYGRFEDLFGTEWSSRLKLTQLPECFLTTPELEECGTPITIPTSNDP |
| | ATGTVRATVDPADGQPQGLAAQSGGGPAVLAATDSASGAGGTYKAT |
| | SLSATGSWTAGGSGGGFSWSYPLTIPDTPAGPAPKISLSYSSQSVDGRT |
| | SVANGQASWIGDGWDYHPGFVERRYRSCNDDRSGTPNNDNSADKE |
| | KSDLCWASDNVVMSLGGSTTELVRDDTTGTWVAQNDTGARIEYKD |
| | KDGGALAAQTAGYDGEHWVVTTRDGTRYWFGRNTLPGRGAPTNSA |
| | LTVPVFGNHTGEPCHAATYAASSCTQAWRWNLDYVEDVHGNAMVV |
| | DWKKEQNRYAKNEKFKAAVSYDRDAYPTQILYGLRADDLAGPPAG |
| | KVVFHAAPRCLESAATCSEAKFESKNYADKQPWWDTPATLHCKAGD |
| | ENCYVTSPTFWSRVRLSAIETQGQRTPGSTALSTVDRWTLHQSFPKQR |
| | TDTHPPLWLESITRVGFGRPDASGNQSSKALPAVTFLPNKVDMPNRV |
| | LKSTTDQTPDFDRLRVEVIRTETGGETHVTYSAPCPVGGTRPTPASNG |
| | TRCFPVHWSPDPAAFSDENLDKSGYEPPLEWFNKYVVTKVTEMDLV |
| | AEQPSVETVYTYEGDAAWAKNTDEYGKPALRTYDQWRGYASVVTR |
| | TGTTANTGAADATEQSQTRTRYFRGMSGDAGRAKVHVTLTDVTGTA |
| | TTVEDLLPYQGMAAETLTYTKAGGDVAARELAFPYSRKTASRARPG |
| | LPALEAYRTGTTRTDSIQHISGDRTRAAQNHTTYDDAYGLPTQTYSLT |
| | LSPNDSGTLVAGDERCTVTTYVHNTAAHIIGLPDRVRATTGDCAAAP |
| | NATTGQIVSDSRTAYDALGAFGTAPVKGLPVQVDTISGGGTSWITSAR |
| | TEYDALGRATKVTDAAGNSTTTTYSPATGPAFEVTVTNAAGHATTTT |
| | LDPGRGSALTVTDQNGRKTTSTYDELGRATGVWTPSRPVNQDASVR |
| | FVYQIEDSKVPAVHTRVLRDAGTYEESIELYDGFLRPRQTQREALGGG |
| | RIVTETLYNANGSAKEVRDGYLAEGEPARELFVPLSLDQVPSATRTA |
| | YDGLGRPVRTTTLHRGVPRHSATTAYGGDWELSRTGMSPDGTTPLSG |
| | SRAVKATTDALGRPARIQHFTTQNVSAESVDTTYTYDPRGPLAQVTD |
| | AQQNTWTYTYDARGRKTSSTDPDAGAAYFGYNALDQQVWSKDNQ |
| | GRLQYTTYDVLGRQTELRDDSASGPLVAKWTFDTLPGAKGHPVAST |
| | RYNDGAAFTSEVTGYDTEYRPTGNKVTIPSTPMTTGLAGTYTYASTY |
| | TPTGKVQSVDLPATPGGLAAEKVITRYDGEDSPTTMSGLAWYTADTF |
| | LGPYGEVLRTASGEAPRRVWTTNVYDEDTRRLTRTTAHRETAPHPVS |
| | TTTYGYDTVGNITSIADQQPAGTEEQCFSYDPMGRLVHAWTDGNSA |
| | VCPRTSTAPGAGPARADVSAGVDGGGYWHSYAFDAIGNRTKLTVHD |
| | RTDAALDDTYTYTYGKTLPGNPQPVQPHTLTQVDAVLNEPGSRVEPR |
| | STYAYDTSGNTTQRVIGGDTQTLAWDRRNKLTSVDTNNDGTPDVKY |
| | LYDASGNRLVEDDGTTRTLFLGEAEIVVNTAGQAVDARRYYSSPGAP |
| | TTIRTTGGKTTGHKLTVMLSDHHSTATTAVELTDTQPVTRRRFDPYG |
| | NPRGTEPTTWPDRRTYLGVGIDDPATGLTHIGAREYDASTGRFISVDP |
| | VMDLTDPLQMNGYTYANADPINNSDPTGLLLDARGGGTQKCVGTCV |
| | KDVTNRKGIPLPPGEEWKHEGEAQTDFNGDGFITVFPTVNVPAKWKK |
| | AKKYTEAFYKAVDTACFYGRESCADPEYPSRAHSINNWKGKACKAV |
| | GGKCPERLSWGEGPAFAGGFAIAAEEYAGRGGYRGGGARRGSPCKC |
| | FLAGTEVLMADGSTKSIEDIKLGDEVVATDPVTGEAGAHPVSALIATE |
| | NDKRFNELVIITSEGVERLTATHEHPFWSPSEGEWLEAGELRTGMTLR |
| | SDSGETLVVAGNRAFTQRARTYNLTVADLHTYYVLAGQTPVLVHNA |
| | NCGPHLKDLQKDYPRRTVGILDVGTDQLPMISGPGGQSGLLKNLPGR |
| | TKANGEHVETHAAAFLRMNPGVRKAVLYIDYPTGTCGTCRSTLPDM |
| | LPEGVQLWVISPRRTEKFTGLPD [SEQ ID NO: 136] |
| DddA | >A4G23_03234 CP017316.1: 3756245-3763321 Streptomyces |
| homolog in | rubrolavendulae strain MJM4426, complete genome |
| Streptomyces | |
| rubrolavendulae | ATGTCCTCGTCCGATGCGGGACGCGCCTTCGGCGTGCCCGAAAAC |
| DNA | GTCCTGGCGCGTTTCACGCGGTATCCCGGCGGGGCGCGACGCCGT |
| | GCCGGGCGCACGGCGCGCGCCCGGCGCCTGGGCATCGTGCTGTCC |
| | GCCGTCCTCTCGGCGACCCTGCTGCCCGCCGAGGCATGGGCCATC |
| | GCGCCCCCGGCGCCGCGCACCGGTCCGACCCTGGACGCCCTCCAG |
| | CAGGAGGAGGAGGTCGATCCGGACCCGGCCGCCATGGAAGAGCT |
| | GGACGACTGGGACGGTGGGCCGGTCGAGCCCCCGGCCGACTACAC |
| | CCCCACCGAGGTCACGCCTCCCACCGGCGGCACCGCCCCGGTGCC |
| | GCTGGACAGCGCGGGCGAGGAACTGGTCCCGGCCGGGACCCTGCC |
| | CGTGCGCATCGGCCAGGCGTCCCCCACCGAGGAGGACCCGGCACC |
| | CCCGGCACCCAGCGGCACGTGGGACGTCACCGTGGAGCCCCGCGC |
| | CACCACCGAGGCGGCCGCCGTGGACGGCGCCATCATCAAGCTCAC |
| | CCCGCCCGCCAGCGGCTCCACACCGGTCGACGTGGAACTCGACTA |
| | CGGCCGGTTCGAGGACCTGTTCGGCACCGAGTGGTCCTCCCGGCTC |
| | AAGCTGACGCAGCTCCCGGAGTGCTTCCTCACGACGCCCGAGCTG |
| | GAGGAGTGCGGCACCCCCATCACCATCCCGACGAGCAACGACCCG |
| | GCCACCGGGACGGTCCGGGCCACCGTCGACCCGGCCGACGGGCAG |
| | CCGCAGGGCCTGGCCGCGCAGTCGGGCGGCGGTCCCGCCGTCCTC |
| | GCCGCGACCGACTCGGCGTCCGGCGCCGGCGGCACGTACAAGGCG |
| | ACCTCCCTCTCGGCCACCGGCTCCTGGACGGCCGGCGGCAGCGGC |
| | GGCGGCTTCTCCTGGTCGTATCCGCTCACCATCCCGGACACCCCGG |
| | CCGGCCCCGCGCCGAAGATCTCCCTGTCGTACTCCTCCCAGTCCGT |
| | CGACGGCCGCACCTCCGTCGCCAACGGCCAGGCGTCGTGGATAGG |
| | CGACGGCTGGGACTACCACCCCGGCTTCGTCGAGCGCCGCTACCG |
| | CTCCTGCAACGACGACCGCTCCGGCACCCCGAACAACGACAACAG |
| | TGCGGACAAGGAGAAGTCCGACCTGTGCTGGGCGAGCGACAACGT |
| | CGTGATGTCGCTCGGCGGCTCCACCACCGAACTCGTCCGCGACGA |
| | CACGACCGGCACGTGGGTCGCGCAGAACGACACCGGTGCCCGGAT |
| | CGAGTACAAGGACAAGGACGGCGGAGCCCTGGCCGCCCAGACCG |
| | CCGGCTACGACGGCGAGCACTGGGTCGTCACCACCCGCGACGGAA |
| | CCCGCTACTGGTTCGGCCGCAACACCCTCCCCGGCCGCGGCGCCCC |
| | CACGAACTCCGCCCTCACCGTCCCCGTCTTCGGCAACCACACCGGC |
| | GAGCCCTGCCACGCCGCCACCTACGCCGCCTCCTCCTGCACCCAGG |
| | CGTGGCGCTGGAACCTCGACTACGTCGAGGACGTCCACGGCAACG |
| | CGATGGTCGTCGACTGGAAGAAGGAGCAGAACCGGTACGCGAAG |
| | AACGAGAAGTTCAAGGCGGCTGTCTCCTACGACCGCGACGCGTAT |
| | CCGACGCAGATCCTCTACGGCCTGCGCGCCGACGACCTGGCGGGC |
| | CCGCCCGCCGGCAAGGTCGTCTTCCACGCCGCCCCGCGCTGCCTCG |
| | AAAGCGCGGCCACCTGCTCCGAAGCCAAGTTCGAGTCCAAGAACT |
| | ACGCGGACAAGCAGCCCTGGTGGGACACACCGGCCACCCTGCACT |
| | GCAAGGCCGGTGACGAGAACTGCTACGTCACCTCGCCGACGTTCT |
| | GGAGCCGCGTCCGCCTGTCGGCGATCGAGACGCAGGGTCAGCGCA |
| | CGCCCGGCTCGACGGCGCTGTCCACGGTCGACCGCTGGACCCTGC |
| | ACCAGTCGTTCCCGAAGCAGCGCACCGACACCCACCCGCCGCTCT |
| | GGCTGGAGTCGATCACCCGCGTGGGCTTCGGCCGGCCGGACGCCT |
| | CCGGCAACCAGTCGAGCAAGGCCCTCCCGGCGGTGACCTTCCTGC |
| | CCAACAAGGTCGACATGCCGAACCGCGTGCTGAAGAGCACGACGG |
| | ACCAGACGCCCGATTTCGACCGCCTGCGCGTCGAGGTCATCCGCA |
| | CGGAGACCGGCGGCGAGACCCATGTGACGTACTCCGCCCCCTGCC |
| | CCGTCGGCGGCACCCGCCCCACCCCGGCCTCCAACGGCACCCGCT |
| | GCTTCCCGGTCCACTGGTCCCCCGACCCGGCGGCCTTCTCCGACGA |
| | GAACCTGGACAAGAGCGGCTACGAGCCGCCCCTCGAGTGGTTCAA |
| | CAAGTACGTCGTCACCAAGGTCACCGAGATGGACCTCGTGGCGGA |
| | GCAGCCCAGCGTCGAGACCGTCTACACCTACGAGGGCGACGCCGC |
| | CTGGGCGAAGAACACCGACGAGTACGGCAAGCCCGCCCTGCGCAC |
| | CTACGACCAGTGGCGCGGCTACGCGAGCGTCGTCACCCGCACGGG |
| | CACCACGGCCAACACCGGCGCCGCCGACGCCACCGAGCAGTCCCA |
| | GACCCGCACCCGGTACTTCCGCGGCATGTCCGGCGACGCGGGCCG |
| | CGCCAAGGTGCACGTCACGCTCACGGACGTGACCGGCACCGCGAC |
| | CACCGTCGAGGACCTGCTCCCGTACCAGGGCATGGCCGCCGAGAC |
| | CCTTACCTACACCAAGGCGGGCGGCGACGTCGCCGCCCGCGAGCT |
| | GGCCTTCCCCTACAGCAGGAAGACCGCCTCCCGCGCCCGCCCCGG |
| | CCTCCCCGCCCTGGAGGCGTACCGCACGGGCACGACGCGCACGGA |
| | CTCCATCCAGCACATCAGCGGCGACCGGACGCGCGCCGCTCAGAA |
| | CCACACCACATACGACGACGCGTACGGCCTGCCCACCCAGACCTA |
| | CTCGCTGACACTCTCGCCGAACGACTCCGGCACCCTTGTCGCCGGT |
| | GACGAGCGGTGCACCGTCACGACGTACGTCCACAACACCGCCGCG |
| | CACATCATCGGCCTCCCCGACCGCGTCCGCGCCACGACGGGCGAC |
| | TGCGCCGCCGCGCCGAACGCCACCACCGGCCAGATCGTCTCCGAC |
| | AGCCGCACCGCGTACGACGCGCTCGGCGCCTTCGGCACGGCCCCG |
| | GTCAAGGGCCTGCCGGTCCAGGTGGACACGATCTCCGGAGGCGGC |
| | ACGAGCTGGATCACCTCGGCGCGCACGGAGTACGACGCGCTGGGC |
| | CGTGCGACCAAGGTCACCGACGCGGCGGGCAACTCCACCACGACC |
| | ACGTACAGCCCGGCGACCGGCCCCGCGTTCGAGGTCACCGTGACC |
| | AACGCGGCTGGTCATGCCACGACCACCACCCTCGACCCCGGTCGC |
| | GGCTCGGCGCTGACCGTCACCGACCAGAACGGCCGCAAGACCACC |
| | AGCACGTACGACGAACTCGGCCGGGCCACCGGCGTGTGGACGCCC |
| | TCCCGCCCGGTGAACCAGGACGCGTCCGTGCGCTTCGTCTACCAG |
| | ATCGAGGACAGCAAGGTCCCGGCGGTGCACACTCGGGTCCTGCGC |
| | GACGCCGGTACGTACGAGGAGTCGATCGAGCTCTACGACGGCTTC |
| | CTCCGCCCCCGTCAGACCCAGCGCGAGGCGCTGGGCGGCGGCCGA |
| | ATCGTCACCGAGACCCTCTACAACGCCAACGGCTCTGCGAAGGAA |
| | GTGCGCGACGGCTACCTGGCGGAGGGCGAGCCCGCGCGGGAACTG |
| | TTCGTCCCGCTCTCCCTCGACCAGGTGCCGAGCGCGACGAGGACG |
| | GCCTATGACGGCCTGGGCCGGCCCGTCCGGACGACGACCCTCCAC |
| | AGGGGAGTCCCCCGGCACTCCGCCACCACGGCGTACGGCGGCGAC |
| | TGGGAACTGAGCCGCACCGGCATGTCGCCCGACGGAACGACGCCG |
| | CTCTCTGGCAGCCGCGCCGTGAAGGCGACGACGGACGCGCTCGGC |
| | CGCCCGGCCCGCATCCAGCACTTCACCACCCAGAACGTGTCGGCC |
| | GAGAGCGTCGACACCACGTACACCTACGACCCCCGCGGCCCCCTT |
| | GCCCAGGTCACCGACGCCCAGCAGAACACCTGGACGTACACGTAC |
| | GACGCCCGTGGGCGCAAGACGTCCTCCACCGACCCGGACGCGGGC |
| | GCCGCCTACTTCGGCTACAACGCGCTGGACCAGCAGGTCTGGTCG |
| | AAGGACAACCAGGGCCGCCTGCAGTACACGACGTACGACGTCCTG |
| | GGCCGCCAGACCGAGCTGCGCGACGACTCCGCGTCCGGCCCGCTG |
| | GTGGCGAAGTGGACCTTCGACACCCTGCCGGGCGCCAAGGGCCAC |
| | CCGGTCGCGTCGACCCGCTACAACGACGGCGCCGCGTTCACCAGC |
| | GAGGTGACCGGTTACGACACCGAGTACCGTCCGACCGGCAACAAG |
| | GTCACCATCCCCAGCACCCCGATGACCACGGGCCTCGCCGGCACG |
| | TACACGTACGCCAGCACGTACACCCCGACCGGCAAGGTCCAGTCC |
| | GTCGACCTGCCCGCGACGCCCGGCGGGCTCGCCGCGGAGAAGGTG |
| | ATCACCCGCTACGACGGCGAGGACTCGCCCACCACGATGTCGGGC |
| | CTGGCCTGGTACACGGCCGACACCTTCCTCGGCCCGTACGGGGAA |
| | GTGCTGCGCACGGCGTCGGGCGAGGCCCCGCGCCGCGTGTGGACG |
| | ACCAACGTCTACGACGAGGACACCCGCCGCCTCACCAGGACCACC |
| | GCGCACCGGGAGACGGCTCCCCACCCGGTCAGCACGACCACCTAC |
| | GGCTACGACACGGTCGGCAACATCACGTCCATCGCCGACCAGCAG |
| | CCGGCGGGTACCGAGGAGCAGTGCTTCTCGTACGACCCGATGGGG |
| | CGCCTCGTCCACGCCTGGACGGACGGCAACAGCGCCGTCTGCCCC |
| | AGGACGTCCACGGCACCGGGCGCCGGCCCGGCCCGCGCCGACGTC |
| | TCGGCCGGTGTCGACGGCGGCGGATACTGGCACTCGTACGCGTTC |
| | GACGCGATCGGCAACCGGACGAAGCTGACCGTCCACGACCGCACC |
| | GACGCGGCCCTGGACGACACGTACACCTACACCTACGGCAAGACC |
| | CTGCCGGGTAACCCGCAGCCGGTCCAGCCGCACACCCTCACCCAG |
| | GTCGACGCGGTGCTCAACGAGCCCGGATCGAGAGTCGAACCGCGC |
| | TCCACATACGCCTACGACACCTCCGGCAACACCACCCAGCGCGTC |
| | ATCGGCGGCGACACCCAGACCCTGGCCTGGGACCGCCGCAACAAG |
| | CTGACGTCCGTCGACACGAACAACGACGGCACACCGGACGTGAAG |
| | TACCTGTACGACGCGTCGGGCAACCGCCTGGTCGAGGACGACGGC |
| | ACCACGCGCACCCTCTTCCTCGGCGAGGCCGAGATCGTCGTCAAC |
| | ACGGCCGGCCAGGCCGTGGACGCGCGCCGCTACTACAGCAGCCCC |
| | GGCGCCCCGACGACGATCCGCACGACCGGCGGCAAGACCACGGG |
| | CCACAAGCTGACCGTCATGCTGTCGGACCACCACAGCACGGCGAC |
| | GACCGCGGTCGAGCTGACCGACACCCAGCCGGTCACCCGCCGCCG |
| | CTTCGACCCGTACGGCAACCCCCGCGGCACCGAGCCGACCACCTG |
| | GCCCGACCGCCGCACCTACCTGGGCGTCGGCATCGACGACCCCGC |
| | CACGGGCCTGACCCACATCGGCGCCCGCGAATACGACGCATCGAC |
| | GGGCCGCTTCATCTCCGTCGATCCGGTCATGGACCTCACGGACCCG |
| | CTCCAGATGAACGGGTACACCTACGCCAACGCGGACCCGATCAAC |
| | AACAGCGACCCCACCGGACTGTTGCTCGACGCCCGAGGCGGCGGC |
| | ACTCAGAAGTGCGTGGGAACCTGCGTCAAGGACGTCACGAACCGA |
| | AAGGGAATTCCGCTCCCGCCTGGCGAGGAGTGGAAGCATGAAGGG |
| | GAGGCGCAAACCGATTTCAACGGTGACGGCTTCATCACCGTCTTCC |
| | CGACCGTGAATGTTCCGGCGAAGTGGAAGAAGGCGAAGAAGTAC |
| | ACGGAGGCTTTCTACAAGGCGGTTGATACTGCTTGCTTCTATGGAC |
| | GCGAAAGCTGTGCGGATCCGGAGTACCCTTCGCGGGCGCATAGCA |
| | TCAACAACTGGAAGGGAAAGGCATGCAAAGCCGTAGGGGGAAAA |
| | TGCCCTGAGAGGTTGTCGTGGGGGGAGGGTCCGGCGTTCGCTGGT |
| | GGCTTCGCGATAGCAGCGGAAGAGTATGCGGGGAGAGGGGGCTA |
| | CCGGGGCGGTGGGGCGAGGAGGGGGTCGCCCTGTAAGTGCTTCCT |
| | TGCCGGCACCGAGGTGCTCATGGCGGATGGCAGCACTAAAAGTAT |
| | CGAGGACATCAAGCTCGGTGACGAAGTGGTTGCGACTGATCCGGT |
| | AACCGGTGAGGCCGGTGCGCACCCTGTCTCGGCGCTGATCGCCAC |
| | CGAGAACGACAAGCGTTTCAACGAGCTGGTCATTATCACCAGCGA |
| | GGGTGTAGAGCGTCTTACCGCAACGCATGAGCACCCCTTCTGGTC |
| | GCCATCCGAAGGGGAGTGGTTGGAGGCGGGTGAGCTGCGCACTGG |
| | CATGACGCTGCGCTCCGACTCTGGCGAAACTCTCGTAGTCGCAGG |
| | AAACCGCGCCTTCACCCAGCGAGCCCGGACCTACAACCTCACGGT |
| | TGCAGACCTCCACACGTACTATGTGCTGGCGGGCCAGACTCCGGT |
| | ACTGGTTCACAATGCAAACTGTGGACCTCACCTGAAGGACCTGCA |
| | AAAGGACTACCCCCGGCGCACTGTGGGCATCCTTGACGTCGGAAC |
| | TGATCAGCTCCCGATGATTAGCGGCCCAGGTGGCCAGTCGGGACT |
| | TCTCAAGAACCTCCCAGGTCGTACGAAGGCCAACGGGGAGCACGT |
| | GGAGACTCACGCAGCAGCGTTCTTGCGTATGAACCCGGGTGTCAG |
| | AAAGGCCGTGCTCTACATCGACTACCCGACGGGGACCTGCGGAAC |
| | ATGTAGAAGTACATTGCCTGACATGCTGCCCGAGGGTGTTCAGTTG |
| | TGGGTGATCTCGCCGCGTAGGACTGAAAAATTCACGGGACTTCCT |
| | GACTGA [SEQ ID NO: 137] |
| DddA | >AVT32940.1 hypothetical protein C6361_29650 [Plantactinospora sp. BC1] |
| homolog in | MGDRLPAFVDGGDTLGIFSRGGIERDLASGVAGPASSLPKGTPGFNGL |
| Plantactinospora | VKSHVEGHAAALMRQNGIPNAELYINRVPCGSGNGCAAMLPHMLPE |
| sp. BC1 | GATLRVYGPNGYDRTFTGLPD [SEQ ID NO: 138] |
| PROTEIN | |
| DddA | >C6361_29650 CP028158.1: 6764267-6764614 Plantactinospora sp. BC1 |
| homolog in | chromosome, complete genome |
| Plantactinospora | CTGGGTGACCGGCTCCCTGCCTTCGTGGACGGTGGAGACACGTTG |
| sp. BC1 | GGCATCTTTTCTCGCGGAGGTATTGAGCGGGACCTCGCCAGCGGA |
| DNA | GTTGCGGGTCCTGCAAGTAGCCTTCCTAAAGGCACGCCTGGCTTCA |
| | ATGGTCTTGTAAAGAGTCATGTTGAAGGGCATGCGGCTGCGCTAA |
| | TGAGACAAAATGGAATTCCGAACGCTGAGCTGTATATCAACAGAG |
| | TGCCGTGCGGTTCAGGTAATGGCTGCGCAGCGATGTTGCCGCATAT |
| | GCTTCCGGAAGGTGCCACCCTCCGCGTATATGGGCCGAACGGGTA |
| | CGATAGAACCTTCACTGGACTTCCGGACTGA [SEQ ID NO: 139] |
| DddA | >BAJ27137.1 hypothetical protein KSE_13070 [Kitasatospora setae KM- |
| homolog in | 6054] |
| Kitasatospora | MAAVPSAEALAAKRARDTIWTPPNTPLGSQTKSVDGENLVPGRLPGP |
| setae KM- | LEPEPADWTPGGPASVPAPGSADVTLGFDSAEAAAARKATGGAAPAS |
| 6054 | DGAALRAGSLPVVIGAAKDAKSGAHRIRVELVDQAKSRAAHLDSPLI |
| PROTEIN | ALTDTEPDTPPSGRTTKVSLDLKGIGAQTWADRARLVALPACALETP |
| | DRPECQQQTPVQSSVDLRSGLLTAEVILPAATEGTAPPTKSSLGSGTAS |
| | GVVQAGLTTAAPAKAAPTVLAATAGASGSGGSFSATSLSPSAAWGA |
| | GSNVGNFTYSYPIQTPPSLGGTAPSVGLGYDSSAVDGKTSAQNSQSS |
| | WLGEGWGYEAGFIERGYKSCNTAGIANSSDMCWGGQNATLSLAGHS |
| | GTLVRDDTTGVWHLQSDDGTKIEQLTGAPNGLQNGEHWRITTTDGT |
| | QFYFGRNHLPGGDGTDPASNSAFKEPVYSPKSGDPCYNSSTATGSWC |
| | TMGWRWNLDYAVDVHGNLITYTYAQETNYYSRGAGQNSGSGTLTD |
| | YTRAGYLTQIAYGQRLSEQVTAKGAAKAAALITFTAAERCVPSGSITC |
| | TEAQRTTANASYWPDTPLDQVCASTGTCTRAGPTFFTTKRLASLTTQ |
| | VLVSGAYRTVDTWTLTHSFKDPGDGNAKSLWLDSIQRTGTNGQTAV |
| | TMPPVTFTAVMKPNRVDGDLTLKDGTKVTVTPFNRPRLQQVTTETG |
| | GQINVVYTTSSDAAHPACSRLAGTMPAAADGNTLACAPVKWYLPGS |
| | SSPDPVDDWFNKYLISAVTEQDAISGTTLIKATNYTYNGDAAWHRND |
| | AEFTDAKTRTWDGFRGYQSVTSTTGSAYPGEAPRTQQTATYLRGMD |
| | GDVKADGSTRSVQVANPLGGPALTDSPWLAGSSFATQTYDQAGGTV |
| | ISANGSVAGGQQVTATHAQSGGMPALVARYPASQVTTTSKSKLSDGT |
| | WRTNTTVSTSDPAHANRPLSSDDKGDGTPGAELCSTNGYATGTNPM |
| | MLNILAERTVTKGACGTPVTSANTVSSARTLYDGKPYGQAGDLAEST |
| | SALTLDHYDTGGNPVYVHTAASTFDAYGRLTSVSEANGATYDAAGN |
| | QLTAPNLTPATTRTAYTPATGAIATTVTQTTPTGWTTTLTQDPGRAEA |
| | LVSTDANGRATTQQYDGLGRLTAAWSPERATNLTPSQKFSYAVNGT |
| | TGPSVVTSQWLKEAGGYAYKNELYDGLGRLRQVQRTSDTYSGRLIT |
| | DTVYDSHGWPVKTASPYYEKTTAPNSTVYLPQDSQVPAQTWVTFDG |
| | IGRTTRSAFVSYGQQQWATTTAYPGADRTDVTPPNGKYPTSTFTDGR |
| | NQVSALWQYRTATPTGNPADATVTTYTYDAANRPATRKDAAGNTW |
| | SYGYDLRGRQTTVTDPDTGTTTTAYDVNSRAVSTTDGKGNTLVVSY |
| | DLIGRKTGLYQGSIAPANQLAGWTYDTLPGGKGKPTSSTRYVGGAGG |
| | SAYTQAVTGYDAGYRPTGTSVTIPASEGKLAGTYTTGLTYNPVLGTL |
| | KQTDLPAIGAAPAESVMYTYNISGVLQKSYSDTYYVYDVQYDAFGRP |
| | VRTTTGDAGTQVVSTQLDKTDYTYNQAGDVTSVTDVQNGTATDAQ |
| | CFTYDHLGRLTQAWTDTAGSTSTTSGTWTDTSGTVHNSGSSQSVPAL |
| | GACANANGPASTGSPAKLSVGGPSPYWQSYGYDSTGNRTTLVQHDT |
| | TGNTTKDTTTTQTFGPAGSVNTATGAPNTGGGTGGPHALLTSSTTGP |
| | TGTQVTSYQYDQLGNTTAVTETSGTTTLAWNGEDKLASVTKTGQAQ |
| | ATSYLYDADGNQLIRRNPGKTTLNLGSDEVTLDTAANSLTDTRYYSA |
| | PGGISIARTTGPTGASALAYQASDPHGTANVQINVDAAQTTTRRPTDP |
| | FGNPRGTQPAPNTWAGDKGFVGGTKDDTTGLTNLGAREYQPTTGRF |
| | LNPDPLLDAGNPQQWNGYAYSDNDPVNSSDPSGLITNALADGDTYV |
| | ARPAAFCVTMSCVEQTSGPGFWEDKRVGDAVFAAVVQATTQSNGN |
| | GSSQTKKEKGIWGQAWDWTKKNGGAILGALVEGAVESTCFIGAGFA |
| | APATGGITVIAGAAACGAVAGEAGALTTNILTPDADHSVDGITNDMV |
| | VGEITGAAVSAASEGASSLAKPAVRKLLGMEAEEGLEAAGRAATGPC |
| | NSFPAGVTVLLADGTTKPIEQIAQGDQVTATDPQTGTTQAEPVTDTIIG |
| | HDDTEFTDLTLTNDADPRAPPSEITSTTHHPYWNATTSRWTDAGDLK |
| | PGDHVRTPDGTELTVNTVYSYTTQPRTARNLTVADLHTYYVLAGNTP |
| | VLVHNTGPGCGEPGFVSDAANSLSGRRITTGQIFDASGNPIGPEITSGG |
| | GSLADRAQSYLADSPNIRNLPAKARYASADHVEAQYAVWMRENGV |
| | TDASVVINQNYVCGLPLGCQAAVPAILPRGSTMTVWYPGSGSPIVLR |
| | GVG [SEQ ID NO: 140] |
| DddA | >KSE_13070 NC_016109.1: 1451556-1458878 Kitasatospora setae KM-6054 |
| homolog in | DNA, complete genome |
| Kitasatospora | GTGCTGGGGACAGCGGCCGCGCTCGCGGTCATGATGTCCATGGCG |
| setae KM- | GCGGTGCCGTCCGCCGAGGCACTGGCCGCGAAGCGGGCACGCGAC |
| 6054 | ACCATCTGGACGCCGCCCAACACCCCGCTGGGCAGCCAGACCAAG |
| DNA | TCCGTCGACGGCGAGAACCTCGTCCCGGGCCGCCTGCCCGGCCCC |
| | CTGGAGCCGGAACCGGCCGACTGGACACCCGGCGGACCGGCATCC |
| | GTGCCCGCTCCGGGCAGCGCGGACGTCACCCTCGGCTTCGACTCC |
| | GCGGAGGCCGCCGCCGCCCGCAAGGCCACCGGCGGCGCCGCCCCC |
| | GCCTCCGACGGCGCGGCCCTCCGCGCGGGCTCCCTCCCCGTCGTCA |
| | TCGGCGCGGCGAAGGACGCCAAGAGCGGCGCCCACCGGATCCGC |
| | GTCGAGCTCGTGGACCAGGCCAAGAGCCGTGCCGCACACCTCGAC |
| | AGCCCGCTGATCGCACTCACCGACACCGAGCCGGACACCCCGCCC |
| | TCCGGTCGGACCACGAAGGTGTCCCTCGACCTGAAGGGCATCGGC |
| | GCCCAGACCTGGGCGGACCGCGCGCGACTCGTCGCCCTGCCCGCC |
| | TGCGCCCTGGAGACGCCCGACAGGCCCGAGTGCCAGCAGCAGACC |
| | CCCGTGCAGAGCTCCGTCGACCTGCGCTCCGGACTGCTGACGGCC |
| | GAGGTCATTCTGCCCGCCGCCACCGAGGGCACCGCCCCGCCCACC |
| | AAGAGCTCCCTCGGCTCGGGCACCGCCTCCGGCGTCGTCCAGGCC |
| | GGCCTCACCACGGCGGCGCCCGCCAAGGCCGCGCCCACGGTGCTC |
| | GCCGCGACCGCCGGCGCGTCCGGCTCGGGCGGCAGCTTCTCGGCG |
| | ACCTCGCTGTCGCCCTCCGCGGCCTGGGGCGCCGGCTCCAACGTCG |
| | GCAACTTCACCTACTCGTACCCGATCCAGACGCCTCCCTCGCTCGG |
| | CGGGACCGCCCCCTCCGTGGGCCTCGGGTACGACTCGTCCGCCGTC |
| | GACGGGAAGACCTCCGCGCAGAACTCCCAGTCCTCCTGGCTCGGC |
| | GAGGGCTGGGGCTACGAGGCCGGGTTCATCGAGCGCGGCTACAAG |
| | TCCTGCAACACGGCCGGCATCGCGAACTCCTCGGACATGTGCTGG |
| | GGCGGGCAGAACGCCACCCTCTCGCTGGCCGGCCACTCCGGCACC |
| | CTGGTGCGCGACGACACCACCGGCGTCTGGCACCTGCAGAGCGAC |
| | GACGGCACGAAGATCGAACAGCTCACCGGCGCGCCCAACGGCCTG |
| | CAGAACGGCGAGCACTGGCGGATCACCACGACCGACGGCACGCA |
| | GTTCTACTTCGGCCGCAACCACCTGCCCGGCGGCGACGGCACCGA |
| | CCCGGCGAGCAACTCCGCCTTCAAGGAACCGGTGTACTCGCCCAA |
| | GAGCGGCGACCCCTGCTACAACTCCTCCACCGCCACCGGCTCCTG |
| | GTGCACGATGGGCTGGCGCTGGAACCTCGACTACGCCGTCGACGT |
| | CCACGGCAACCTGATCACCTACACCTACGCCCAGGAGACCAACTA |
| | CTACAGCCGAGGCGCCGGCCAGAACAGCGGCAGCGGCACCCTGAC |
| | CGACTACACCCGCGCCGGCTACCTCACCCAGATCGCCTACGGCCA |
| | GCGCCTGAGCGAGCAGGTCACCGCCAAGGGCGCGGCCAAGGCCG |
| | CTGCCCTCATCACCTTCACCGCCGCGGAACGCTGCGTCCCGTCCGG |
| | CTCGATCACCTGCACCGAGGCACAGCGCACGACCGCGAACGCCTC |
| | GTACTGGCCGGACACCCCGCTCGACCAGGTCTGCGCCTCCACCGG |
| | CACCTGCACCCGGGCCGGCCCGACGTTCTTCACCACCAAGCGCCTC |
| | GCCTCCCTCACCACCCAGGTCCTGGTCTCCGGCGCCTACCGCACCG |
| | TCGACACCTGGACGCTCACCCATTCCTTCAAGGACCCGGGCGACG |
| | GCAACGCCAAGTCGCTGTGGCTCGACTCGATCCAGCGCACCGGCA |
| | CCAACGGGCAGACCGCGGTCACCATGCCGCCCGTCACCTTCACGG |
| | CGGTGATGAAGCCGAACCGGGTGGACGGGGACCTCACCCTCAAGG |
| | ACGGCACCAAGGTCACCGTCACCCCGTTCAACCGGCCCCGCCTCC |
| | AGCAGGTCACCACGGAGACCGGCGGCCAGATCAACGTCGTCTACA |
| | CCACCTCCTCCGACGCCGCGCACCCCGCCTGCTCGCGCCTGGCCGG |
| | CACCATGCCCGCCGCGGCGGACGGCAACACCCTCGCCTGCGCCCC |
| | CGTCAAGTGGTACCTGCCCGGATCCAGCTCCCCGGACCCGGTCGA |
| | CGACTGGTTCAACAAGTACCTGATCAGCGCCGTCACCGAACAGGA |
| | CGCGATCAGCGGCACCACCCTGATCAAGGCCACCAACTACACCTA |
| | CAACGGCGACGCCGCCTGGCACCGCAACGACGCCGAGTTCACCGA |
| | CGCCAAGACCCGCACCTGGGACGGCTTCCGCGGCTACCAGTCCGT |
| | CACCAGCACCACCGGCAGCGCCTACCCGGGCGAGGCCCCCAGGAC |
| | CCAGCAGACCGCGACCTACCTGCGCGGCATGGACGGCGACGTCAA |
| | GGCCGACGGCTCCACCCGCAGCGTCCAGGTCGCCAACCCGCTCGG |
| | CGGCCCGGCCCTCACCGACAGCCCGTGGCTGGCCGGCTCCAGCTT |
| | CGCCACCCAGACCTACGACCAGGCCGGCGGCACCGTCATCTCCGC |
| | CAACGGCTCCGTCGCCGGCGGCCAGCAGGTCACCGCCACCCACGC |
| | CCAGAGCGGCGGCATGCCGGCCCTGGTCGCCCGCTACCCCGCCTC |
| | CCAGGTCACCACCACCTCCAAGTCCAAGCTCTCCGACGGGACCTG |
| | GCGCACCAACACCACCGTCAGCACCAGCGACCCCGCGCACGCCAA |
| | CCGCCCCCTCAGCAGCGACGACAAGGGCGACGGCACCCCCGGCGC |
| | CGAACTGTGCAGCACCAACGGCTACGCCACCGGCACCAACCCGAT |
| | GATGCTGAACATCCTCGCCGAGCGGACGGTCACCAAGGGCGCCTG |
| | CGGCACCCCCGTGACCTCGGCCAACACCGTCTCCTCCGCCCGCACC |
| | CTCTACGACGGCAAGCCCTACGGCCAGGCCGGCGACCTCGCCGAG |
| | TCCACCAGCGCCCTGACCCTGGACCACTACGACACCGGCGGCAAC |
| | CCCGTCTACGTCCACACCGCCGCCTCCACCTTCGACGCCTACGGCC |
| | GGCTTACCAGCGTCAGCGAGGCCAACGGCGCCACCTACGACGCCG |
| | CGGGCAACCAGCTCACCGCGCCCAACCTCACCCCCGCCACCACCC |
| | GCACCGCCTACACCCCGGCCACCGGCGCCATCGCCACCACCGTCA |
| | CCCAGACCACGCCCACCGGCTGGACCACCACCCTCACCCAGGACC |
| | CGGGCCGCGCCGAAGCTCTGGTCTCCACCGACGCCAACGGCCGCG |
| | CCACCACCCAGCAGTACGACGGCCTCGGCCGCCTGACCGCCGCCT |
| | GGTCACCGGAGCGCGCGACCAACCTCACCCCCAGCCAGAAGTTCT |
| | CCTACGCGGTCAACGGCACCACCGGCCCCTCCGTCGTCACCTCCCA |
| | GTGGCTCAAGGAAGCCGGCGGCTACGCGTACAAGAACGAGCTGTA |
| | CGACGGCCTCGGCCGCCTGCGCCAGGTCCAGCGCACCAGCGACAC |
| | CTACTCCGGGCGGCTGATCACCGACACCGTCTACGACTCGCACGG |
| | CTGGCCCGTCAAGACCGCCAGCCCGTACTACGAGAAGACCACCGC |
| | GCCCAACAGCACCGTCTACCTGCCGCAGGACTCCCAGGTGCCCGC |
| | CCAGACCTGGGTCACCTTCGACGGCATCGGCCGGACCACCCGCTC |
| | CGCGTTCGTCTCCTACGGACAGCAGCAGTGGGCCACCACCACCGC |
| | CTACCCCGGCGCCGACCGCACCGACGTCACCCCGCCCAACGGCAA |
| | ATACCCGACCAGCACCTTCACCGACGGCCGCAACCAGGTCAGCGC |
| | CCTGTGGCAGTACCGCACCGCCACCCCCACCGGCAACCCGGCCGA |
| | CGCGACCGTCACCACCTACACCTACGACGCCGCCAACCGGCCCGC |
| | CACCCGCAAGGACGCCGCCGGGAACACCTGGAGCTACGGCTACGA |
| | CCTGCGCGGCCGCCAGACCACCGTCACCGACCCCGACACCGGCAC |
| | CACCACCACCGCCTACGACGTCAACTCGCGCGCCGTCTCCACCACC |
| | GACGGCAAGGGCAACACCCTCGTCGTCAGCTACGACCTGATCGGC |
| | CGCAAGACCGGCCTCTACCAGGGCAGCATCGCCCCGGCCAACCAG |
| | CTCGCCGGCTGGACGTACGACACCCTGCCGGGCGGAAAGGGCAAG |
| | CCCACCTCCTCCACCCGCTACGTCGGGGGCGCCGGCGGCTCGGCCT |
| | ACACCCAGGCCGTCACCGGCTACGACGCCGGCTACCGGCCCACCG |
| | GCACCTCGGTGACGATCCCCGCCAGCGAAGGCAAGCTCGCCGGTA |
| | CCTACACCACCGGCCTGACGTACAACCCGGTCCTCGGCACGCTCA |
| | AGCAGACCGACCTGCCGGCCATCGGCGCGGCGCCCGCCGAGAGCG |
| | TCATGTACACCTACAACATCTCCGGCGTCCTGCAGAAGTCCTACAG |
| | CGACACCTACTACGTCTACGACGTGCAGTACGACGCCTTCGGCCG |
| | CCCGGTCCGCACGACCACCGGCGACGCCGGAACCCAGGTCGTCTC |
| | CACCCAGCTCGACAAGACCGACTACACCTACAACCAGGCCGGCGA |
| | CGTCACCTCGGTCACCGACGTCCAGAACGGCACCGCCACCGACGC |
| | CCAGTGCTTCACCTACGACCACCTCGGGCGCCTCACCCAGGCCTGG |
| | ACCGACACCGCGGGCTCCACCAGCACCACCAGCGGCACCTGGACC |
| | GACACCTCCGGCACCGTCCACAACAGCGGCTCCTCCCAGTCCGTCC |
| | CCGCACTCGGCGCCTGCGCCAACGCCAACGGCCCCGCCAGCACCG |
| | GCAGCCCCGCCAAGCTCTCCGTCGGCGGCCCCTCCCCGTACTGGCA |
| | GAGCTACGGCTACGACAGCACCGGCAACCGCACCACCCTCGTCCA |
| | GCACGACACCACCGGCAACACCACCAAGGACACCACCACCACCCA |
| | GACCTTCGGCCCCGCCGGATCGGTCAACACCGCCACCGGCGCCCC |
| | CAACACCGGCGGCGGCACCGGCGGCCCGCACGCCCTGCTCACCAG |
| | CAGCACCACCGGACCCACCGGGACCCAGGTCACCAGCTACCAGTA |
| | CGACCAGCTCGGCAACACCACCGCGGTCACCGAGACGTCCGGAAC |
| | CACCACCCTCGCCTGGAACGGCGAGGACAAGCTCGCCTCCGTCAC |
| | CAAGACCGGCCAGGCCCAGGCCACCAGCTACCTCTACGACGCCGA |
| | CGGCAACCAGCTCATCCGCCGCAACCCCGGCAAGACCACCCTCAA |
| | CCTCGGCAGCGACGAGGTCACCCTCGACACCGCCGCCAACTCCCT |
| | CACCGACACCCGCTACTACAGCGCCCCCGGCGGCATCAGCATCGC |
| | CCGCACCACCGGACCCACCGGCGCAAGCGCCCTCGCCTACCAGGC |
| | CTCCGACCCCCACGGCACCGCCAACGTCCAGATCAACGTCGACGC |
| | CGCCCAGACCACCACCCGCCGCCCCACCGACCCCTTCGGCAACCC |
| | CCGCGGCACCCAGCCCGCCCCCAACACCTGGGCCGGCGACAAGGG |
| | CTTCGTCGGCGGCACCAAGGACGACACCACCGGACTCACCAACCT |
| | CGGCGCCCGCGAATACCAACCCACCACCGGCCGCTTCCTCAACCC |
| | CGACCCACTCCTCGACGCCGGCAACCCCCAGCAGTGGAACGGCTA |
| | CGCCTACAGCGACAACGACCCCGTCAACAGCTCCGACCCCAGCGG |
| | ACTCATCACCAACGCCCTGGCCGACGGCGACACCTACGTCGCCCG |
| | CCCCGCCGCCTTCTGCGTCACCATGTCGTGCGTCGAGCAGACCAGC |
| | GGCCCCGGTTTCTGGGAGGACAAGCGCGTCGGTGACGCCGTCTTC |
| | GCCGCCGTCGTCCAGGCCACCACGCAGAGCAACGGCAACGGGTCA |
| | TCCCAGACCAAGAAAGAGAAGGGCATCTGGGGCCAGGCCTGGGA |
| | CTGGACCAAGAAGAACGGCGGCGCCATCCTCGGAGCGCTGGTAGA |
| | GGGAGCGGTCTTCAGCACATGCTTCATCGGAGCTGGATTCGCCGC |
| | ACCTGCAACGGGAGGAATCACCGTCATCGCCGGTGCTGCGGCCTG |
| | CGGGGCTGTGGCCGGCGAGGCAGGGGCACTGACCACCAATATCCT |
| | CACCCCAGATGCCGACCACTCCGTCGACGGCATCACCAACGACAT |
| | GGTCGTTGGTGAAATCACCGGGGCGGCTGTCAGCGCAGCGAGCGA |
| | GGGCGCAAGCTCCCTCGCCAAGCCGGCGGTCCGCAAACTCCTGGG |
| | CATGGAAGCCGAGGAAGGACTCGAGGCAGCAGGCCGCGCCGCCA |
| | CCGGACCTTGCAACAGTTTCCCGGCCGGCGTCACCGTCCTCCTCGC |
| | CGACGGCACCACCAAGCCCATCGAACAGATCGCCCAGGGCGACCA |
| | GGTAACCGCCACCGACCCGCAGACAGGCACCACCCAGGCAGAACC |
| | CGTCACCGACACGATCATCGGCCACGACGACACGGAATTCACCGA |
| | CCTCACCCTCACCAACGACGCAGACCCCCGCGCCCCGCCCAGCGA |
| | GATCACCTCCACCACCCACCACCCCTACTGGAACGCCACCACCAG |
| | CCGCTGGACCGATGCCGGCGACCTCAAGCCCGGCGACCACGTCCG |
| | CACCCCCGACGGCACCGAACTGACCGTCAACACCGTCTACAGCTA |
| | CACCACACAACCCCGGACCGCGCGCAACCTCACCGTCGCAGACCT |
| | CCACACGTACTATGTGCTCGCTGGAAATACGCCGGTCCTAGTGCAT |
| | AACACCGGCCCGGGATGTGGTGAGCCGGGATTCGTTAGTGACGCT |
| | GCTAATTCTCTCTCGGGCAGGCGCATCACCACGGGACAAATATTTG |
| | ATGCGAGCGGGAATCCGATCGGGCCTGAGATCACGAGCGGCGGCG |
| | GCAGTCTGGCAGATAGGGCGCAGAGTTATCTTGCCGACTCCCCTA |
| | ATATTCGAAATCTGCCCGCTAAGGCGAGATATGCGTCGGCTGACC |
| | ACGTTGAGGCGCAATATGCAGTGTGGATGCGAGAAAATGGAGTGA |
| | CCGACGCCAGTGTGGTCATCAATCAAAACTATGTATGTGGGCTGC |
| | CCCTAGGCTGCCAGGCGGCGGTGCCCGCTATCCTCCCTCGCGGCTC |
| | GACCATGACGGTATGGTATCCAGGGTCAGGAAGTCCCATCGTATT |
| | GCGGGGAGTGGGTTAA [SEQ ID NO: 141] |
| DddA | >ATE59819.1 type IV secretion protein Rhs [Thauera sp. K11] |
| homolog in | MRAFRLIACLLAFSAAAAPAAADTSSMLGRLPEASARQLKERLAPRG |
| Thauera sp. | LASAAALRQYLDASQRELDTAPEADDVPARSQRFAARAGELTALREQ |
| K11 | ARRDLASLEDAAKASGSAEATQRIGRIRGQVDARFDRLEGLFTTWRN |
| PROTEIN | APQGSERRQARRELRAALATLRHAGTPAPAAIPVPTLGPLQPAGEPAA |
| | NPPAARLPAYAQADDATGDPFTPGGFRLMKVAALPPAVAAEAATDC |
| | SATSADLADDGKDVRLTQPIRDLAASLDYSPARILRWTQQNVAFEPY |
| | WGALKGAEGVLQTRAGNSTDQASLLIALLRASNIPARYVRGTVQLND |
| | TAAQDDAGGRAQRWLGTKRYRASAAVLAGGGTSAGLQSIDGTVRGI |
| | RFSHVWVQACVPHGAYRGARAEAGGYRWLALDAAVKDHDYQQGI |
| | AVDVPLTDAAFYTPYLAARSDQLPHEHFAQKVAEAARATDANAALA |
| | DVPYAGTPRPLRYDVLPGSLPYEVEAFTNWPGLGSSETASLPDAHRH |
| | TFTVTVRNGATTLASAALPYPQNAFKRVTLSYQPTAASQAAWNAWT |
| | GDLPAAADGSIQVVPQIKADGTVLAAGAPANALPLAGVHNVILKVSQ |
| | GERSGAACINDSGNPADPKDTDGTCLNKTVYTNIKAGAYHALGLNA |
| | LHTSNAFLGQRLEALAAGVQAYPVAPTPAAGAGYEATVGELLHLVL |
| | QDYLHQTEQADQRNAALRGFKSVGPYDLGLTASDLETDYLFDIPVAI |
| | KPAGVFVDFKGGLYGFVKLDTTAETAAARAAENVDLAKLSIYSGSAL |
| | EHHVWQQALRTDAVSTVRGLQFAAEQGIPLVTFTAANIGQYDSLMQ |
| | MSGATSMAAYKSAIQNAVKGSDNGNHGVVTVPRAQIAYADPVDPAS |
| | KWTGAVYMSQNPVTGEYGAIINGTIAGGFPLLNSTPFSNLYNFDSFVP |
| | NTLLGTNGGAGAVQTLPGGTQGESSWITKAGDPVNMLTGNYTLQAR |
| | DFTIKGRGGLPIVLERWFNAQNATDGPFGFGWTHSFNHQLRFYGIESG |
| | QSKVGWVDGTGAQRFYAVAAAGSIAPGTTLAAQAGVFTTLSRLADG |
| | RFQVRETNGLTYSFESLTSPTTPPAAGSEPRARLLAIADRHGNTLTLNY |
| | SGSQLASVSDSLGRTVLSFTWNGNRIGKVKDVSGREVNYAYEDGNG |
| | NLTRVTDPLGQATRYSYYTSADGAKLDHALRRHTLPRGNGMEFEYY |
| | AGGQVFRHTPFDTSGNLIPESALTFHYNSYRRESWTVDGRGAEERFLF |
| | DTHGNVIQQTAANGATHTYAYADPNDPHLRTRMTDPVGRVTQYSYT |
| | AEGYLQTLTLPSGAVQAWRDYDAFGQPRRVKDARGNWTLHHYDTA |
| | GTRTDSIRVKSGVVPTVGTAPAAANVVSWIKYQGDSVGNLTGVKRL |
| | RDWTGATLGNFASGSGPVVTTTFDAARLNVASVGRSGNRNGSQISET |
| | SPIFSHDALGRLTGGVDGRWYPVAFDYDVLDRVTRATDATGQPRRY |
| | AFDVNGNRIGTELIAGGSRIDSSVAAFDVQDRVAHVLDHAGNRVAYA |
| | YDAVGNRVSVESPDGYAIGFDYDLAGRPYSAYDEDGNRVFSAFDVA |
| | GRVRAVIDPNGAATLYDYHGDEQDGRLARVEQPAIPGQNAGRAAET |
| | DYDAGGLPIRVRQVSAGGEAREGYRFHDELGRVVRSVSAPDDVGQR |
| | LQVCYSYDALSNLTQVRAGATTDTTSAACAGSPAVQLTQSWDDFGN |
| | LLTRTDALGRVWKFEYDAHGNLVASQTPEQAKVSTRSTYRYDPALH |
| | GLLAGRSVPGSGSAGQSVSYARNALGQVIRAETRDGAGNLVVAYDY |
| | QYDAAHRVVRIVDSRGGKALDYAWTPAGRLASITLDGHVWRFQYD |
| | GVGRLAAIVAPNGATIAMARDAAGRLTERRWPDGAKSAFDWLPEGS |
| | LAAIEHSAGGSALAQFAYAYDAWGNRTSATETLAGTSRSLAYGYDA |
| | LDRLKTVTTDGATETHAFDLFGNRTSKTTGGVTTDYLFDAAHQLTQV |
| | QIAGTPTERLAYDDNGNLRKHCVGSPSGSTSDCTGTTVLSLAWNGLD |
| | QLIQAARTGLPAESYAYDDAGRRVTKAVGSSATHFAYDGPDILAEYA |
| | SPAGSPTAVYAHGAGIDEPLLRLTGATSTPAASAHHYAQDGLGSIVA |
| | AYGEIGASGPVSAASVSATHSYSAGSYPPAKLIDGETTGSTGFWAGSS |
| | GNFAADPAVITLELGAEKSVSRVRLHRVASYLPDYVVKDAEVQVRKP |
| | DNSWQTVGTLTNNTSEDSPEIVLTGAPGSALRVLVKGVRNGSLVLMA |
| | EVTMSADGGAASVATARYDAWGNVTQASGSIPAFGYTGREPDATGL |
| | VYYRARYYHPALGRFASRDPLGLAAGINPYAYAGGNPILYNDPDGLL |
| | AQLAWNTAASYWGQPIVQETVATIRNGAAVAAGNFVPDTVNGATG |
| | WFEQFLHQESGSFGRMDSWVDVRNPVAQDVAQDLRGVAAVGLMM |
| | TPLRYGRASNASFNPPVANLPLNTGGKTSGMLHIPGQESLSLTSGIAGP |
| | SQVVRGQGLPGFNGNQLTHVEGHAAAYMRTHKVSEAVLDINKAPCT |
| | AGSGGGCNGLLPRMLPEGAHLTIRHPNGVQVYIGTPD [SEQ ID NO: |
| | 142] |
| DddA | >CCZ27_07525 NZ_CP023439.1: 1708666-1716450 Thauera sp. K11 |
| homolog in | chromosome, complete genome |
| Thauera sp. | ATGCGTGCCTTCCGCCTGATCGCCTGCCTTCTCGCCTTTTCGGCGG |
| K11 | CAGCCGCACCTGCTGCGGCTGACACGTCGTCGATGCTGGGGCGTC |
| DNA | TGCCTGAAGCAAGCGCCCGCCAGCTCAAGGAGCGGTTGGCGCCGC |
| | GTGGCCTTGCCTCCGCTGCCGCCTTGCGCCAGTACCTGGACGCCTC |
| | GCAACGCGAGCTGGACACCGCACCGGAAGCGGACGACGTACCCG |
| | CCCGCAGCCAACGCTTTGCCGCAAGGGCGGGCGAACTCACCGCGC |
| | TGCGCGAACAGGCGCGCCGGGATCTCGCCAGTCTGGAGGACGCCG |
| | CGAAGGCGAGCGGCTCGGCCGAGGCGACGCAGCGCATCGGTCGA |
| | ATCCGCGGGCAGGTGGACGCACGCTTCGACCGGCTCGAAGGGCTT |
| | TTTACCACTTGGCGCAATGCGCCCCAGGGCAGCGAACGCCGCCAG |
| | GCCCGCCGCGAACTGCGTGCCGCGCTCGCCACGCTCCGCCATGCC |
| | GGCACCCCGGCTCCGGCTGCGATTCCTGTTCCTACCCTCGGCCCCC |
| | TGCAACCGGCCGGCGAGCCGGCTGCCAACCCACCGGCCGCGCGCT |
| | TGCCAGCCTATGCGCAAGCGGATGACGCGACTGGCGACCCCTTTA |
| | CCCCCGGTGGCTTCCGGCTGATGAAGGTCGCCGCACTGCCGCCGG |
| | CGGTCGCGGCCGAGGCGGCAACGGACTGCTCCGCCACCAGCGCCG |
| | ACCTGGCCGACGACGGCAAGGACGTGCGCCTGACCCAGCCGATCC |
| | GCGACCTCGCGGCATCGCTCGACTACTCACCGGCACGCATCCTGC |
| | GCTGGACGCAGCAGAACGTCGCCTTCGAACCCTACTGGGGGGCAC |
| | TCAAGGGGGCGGAAGGCGTGCTGCAGACGCGCGCCGGCAACAGC |
| | ACCGACCAGGCCAGCCTGCTGATCGCACTCTTGCGGGCCTCCAAC |
| | ATTCCCGCCCGCTACGTACGCGGCACCGTGCAGCTCAACGACACT |
| | GCCGCGCAGGACGACGCAGGCGGGCGGGCGCAGCGCTGGCTGGG |
| | CACCAAGCGCTACCGTGCATCGGCCGCGGTACTCGCCGGCGGCGG |
| | AACTTCCGCCGGCCTGCAGTCGATCGACGGCACCGTCCGCGGCAT |
| | CCGCTTCAGCCATGTCTGGGTCCAGGCCTGCGTTCCCCATGGCGCT |
| | TACCGCGGTGCCCGCGCGGAAGCCGGCGGCTATCGCTGGCTGGCG |
| | CTGGACGCGGCGGTGAAGGACCATGACTACCAGCAGGGCATCGCG |
| | GTCGATGTGCCGCTCACCGATGCCGCGTTCTACACGCCCTATCTGG |
| | CGGCGCGCAGCGACCAGTTGCCGCACGAGCATTTCGCACAGAAGG |
| | TGGCGGAGGCGGCGCGTGCGACCGACGCCAATGCGGCGCTGGCCG |
| | ACGTGCCCTACGCCGGTACGCCGCGGCCGCTGCGCTACGACGTGC |
| | TGCCCGGTTCGCTGCCCTACGAGGTCGAAGCCTTCACCAACTGGCC |
| | CGGCCTCGGTTCGTCCGAAACCGCAAGCCTGCCGGACGCACACCG |
| | CCACACCTTCACCGTGACGGTCAGGAACGGCGCCACCACGTTGGC |
| | GAGCGCCGCGCTGCCCTATCCGCAGAACGCCTTCAAGCGCGTCAC |
| | GCTGTCCTATCAGCCGACTGCCGCCTCGCAGGCGGCCTGGAACGC |
| | CTGGACGGGCGATCTGCCCGCCGCGGCCGACGGCAGCATCCAGGT |
| | CGTGCCGCAGATCAAGGCCGACGGTACCGTGCTCGCCGCAGGTGC |
| | GCCCGCCAACGCGCTGCCGCTCGCCGGCGTGCACAACGTCATCCT |
| | CAAGGTCTCGCAGGGCGAGCGCAGCGGTGCCGCGTGCATCAACGA |
| | CAGCGGCAACCCCGCCGACCCGAAGGACACCGACGGCACCTGCCT |
| | CAACAAGACCGTCTACACCAACATCAAGGCCGGCGCCTACCACGC |
| | CCTGGGCCTGAATGCGCTGCACACCTCGAATGCCTTCCTCGGCCAG |
| | CGGCTCGAAGCGCTGGCGGCCGGCGTGCAGGCCTATCCCGTCGCG |
| | CCCACGCCGGCCGCGGGTGCCGGCTACGAGGCCACGGTCGGTGAA |
| | TTGCTGCATCTGGTGCTGCAGGACTACCTGCACCAGACCGAGCAG |
| | GCCGACCAGCGCAACGCCGCGTTGCGCGGCTTCAAGAGCGTGGGG |
| | CCGTACGACCTCGGGCTGACCGCGTCCGACCTCGAAACCGACTAC |
| | CTCTTCGACATCCCGGTCGCGATCAAGCCGGCCGGCGTGTTCGTGG |
| | ACTTCAAGGGCGGCCTCTACGGTTTCGTCAAACTCGATACCACGGC |
| | CGAGACGGCCGCGGCACGCGCCGCCGAAAACGTGGATCTGGCCAA |
| | GCTCTCGATCTACTCCGGCTCCGCGCTCGAACACCACGTCTGGCAG |
| | CAGGCGCTGCGCACCGATGCGGTGTCCACCGTGCGTGGGCTGCAG |
| | TTCGCCGCCGAGCAGGGCATTCCGCTCGTCACCTTCACCGCGGCCA |
| | ACATCGGCCAGTACGACAGCCTCATGCAGATGAGCGGCGCCACCA |
| | GCATGGCCGCTTACAAGAGCGCGATCCAGAACGCGGTGAAGGGCT |
| | CGGACAACGGCAACCACGGCGTCGTCACCGTGCCGCGCGCCCAGA |
| | TCGCCTACGCCGACCCCGTCGATCCGGCGAGCAAATGGACCGGCG |
| | CGGTCTACATGTCTCAGAACCCCGTCACCGGAGAGTACGGGGCGA |
| | TCATCAACGGCACCATCGCCGGCGGCTTCCCGCTGCTCAACAGCA |
| | CGCCCTTCAGCAATCTCTACAACTTCGATTCCTTCGTGCCCAACAC |
| | CCTCCTTGGCACGAACGGGGGTGCCGGTGCGGTGCAGACCCTGCC |
| | CGGCGGCACCCAGGGCGAGAGTTCCTGGATCACCAAGGCCGGCGA |
| | CCCGGTGAACATGCTCACCGGCAACTACACGCTGCAGGCACGCGA |
| | CTTCACCATCAAGGGCCGGGGCGGACTGCCGATCGTGCTGGAGCG |
| | CTGGTTCAACGCGCAGAACGCCACCGACGGGCCGTTCGGCTTCGG |
| | CTGGACGCACAGCTTCAACCATCAGTTGCGTTTCTACGGCATCGAG |
| | AGCGGCCAGTCCAAGGTCGGCTGGGTGGACGGCACTGGCGCCCAG |
| | CGCTTCTACGCCGTGGCCGCCGCCGGCAGCATTGCGCCGGGCACG |
| | ACGCTGGCCGCGCAGGCCGGGGTGTTCACGACGCTGTCGCGTCTG |
| | GCCGACGGCCGCTTCCAGGTGCGCGAGACCAACGGCCTCACCTAC |
| | AGCTTCGAATCGCTCACGAGCCCGACCACCCCGCCGGCCGCGGGC |
| | AGCGAACCGCGCGCAAGACTGCTGGCCATCGCCGACCGCCACGGC |
| | AACACCCTGACGCTCAACTACAGCGGCAGCCAGCTTGCCTCGGTG |
| | AGCGACAGCCTCGGCCGCACGGTGCTCAGCTTCACCTGGAACGGC |
| | AACCGCATCGGCAAGGTGAAGGACGTCAGCGGACGGGAAGTGAA |
| | CTACGCCTACGAGGACGGCAACGGCAACCTCACGCGCGTCACCGA |
| | TCCGCTGGGTCAAGCCACGCGCTACAGCTACTACACCAGTGCCGA |
| | CGGTGCCAAGCTCGACCACGCCCTGCGCCGCCACACCCTGCCGCG |
| | CGGCAACGGCATGGAGTTCGAGTACTACGCCGGTGGCCAGGTCTT |
| | CCGCCACACGCCGTTCGACACCAGCGGCAACCTCATTCCCGAATC |
| | GGCGCTGACCTTCCACTACAACAGTTATCGGCGCGAGAGCTGGAC |
| | GGTCGATGGCCGCGGTGCCGAGGAGCGCTTCCTGTTCGACACGCA |
| | CGGCAACGTGATCCAGCAGACCGCCGCCAACGGTGCCACCCACAC |
| | CTACGCGTACGCCGACCCGAACGATCCGCATCTGCGCACGCGCAT |
| | GACAGACCCGGTCGGCCGCGTCACCCAGTACAGCTATACCGCCGA |
| | AGGCTATCTGCAGACCCTGACGCTGCCGTCGGGCGCCGTGCAGGC |
| | GTGGCGCGACTACGACGCCTTCGGCCAGCCCCGCCGCGTCAAGGA |
| | CGCGCGCGGCAACTGGACGCTCCACCACTACGACACCGCCGGGAC |
| | ACGGACCGACTCCATCCGGGTCAAATCGGGCGTGGTCCCCACCGT |
| | CGGCACCGCGCCTGCCGCGGCCAACGTCGTTTCCTGGATCAAGTA |
| | CCAGGGCGACAGCGTGGGCAACCTCACCGGCGTCAAGCGCCTGCG |
| | CGACTGGACGGGCGCGACCCTGGGCAATTTCGCCAGCGGCAGCGG |
| | CCCCGTCGTCACCACCACCTTCGATGCGGCCAGGCTCAACGTCGCC |
| | AGCGTCGGCCGTAGCGGCAACCGCAACGGCAGCCAGATCAGCGA |
| | GACCAGCCCGATCTTCTCCCACGACGCGCTGGGGCGCCTCACCGG |
| | CGGGGTGGACGGGCGCTGGTATCCGGTCGCCTTCGATTACGACGT |
| | GCTCGACCGCGTCACCCGCGCCACCGACGCCACGGGCCAGCCGCG |
| | CCGCTACGCGTTCGACGTCAACGGCAACCGCATCGGTACGGAGCT |
| | GATTGCCGGCGGCAGCCGTATCGATTCCTCGGTGGCCGCCTTCGAC |
| | GTGCAGGACCGCGTCGCCCACGTCCTCGATCACGCCGGCAACCGC |
| | GTGGCCTACGCCTACGATGCGGTGGGCAACCGGGTGAGCGTGGAA |
| | AGCCCCGACGGCTACGCCATCGGCTTCGACTACGACCTCGCCGGA |
| | CGGCCCTATTCGGCCTACGACGAAGACGGCAACCGCGTCTTCTCC |
| | GCCTTCGACGTGGCCGGGCGCGTGCGAGCGGTCATCGACCCCAAC |
| | GGCGCCGCGACGCTCTACGACTATCACGGCGACGAGCAGGACGGG |
| | CGTCTCGCGCGCGTGGAGCAGCCCGCCATCCCGGGCCAGAACGCG |
| | GGCCGCGCCGCCGAGACCGACTACGATGCGGGTGGGTTGCCCATC |
| | CGCGTGCGCCAGGTCTCGGCCGGCGGCGAAGCGCGCGAAGGCTAC |
| | CGTTTCCACGACGAGCTTGGCCGCGTGGTGCGCAGCGTCTCCGCGC |
| | CGGACGACGTCGGCCAGCGGCTGCAGGTCTGCTACAGCTACGATG |
| | CACTCTCGAACCTCACCCAGGTGCGCGCCGGCGCCACCACCGACA |
| | CCACCAGTGCCGCCTGCGCCGGCAGCCCCGCGGTGCAGCTCACCC |
| | AGAGCTGGGACGACTTTGGCAACCTGCTGACGCGCACCGACGCGC |
| | TGGGCCGGGTGTGGAAGTTCGAGTACGACGCCCACGGCAACCTCG |
| | TCGCCAGCCAGACGCCCGAGCAGGCCAAGGTCTCGACGCGCAGCA |
| | CCTACCGCTACGATCCGGCGCTGCACGGCTTGCTGGCCGGGCGCA |
| | GCGTGCCGGGCAGCGGCAGTGCGGGCCAGAGCGTGAGCTATGCGC |
| | GCAACGCGCTCGGCCAGGTCATCCGCGCCGAGACGCGCGACGGCG |
| | CGGGCAACCTCGTCGTCGCCTACGACTACCAGTACGACGCCGCCC |
| | ACCGTGTGGTGCGCATCGTCGACAGCCGCGGCGGCAAGGCGCTCG |
| | ACTACGCCTGGACGCCCGCCGGGCGGCTGGCGAGCATTACCCTGG |
| | ACGGCCATGTCTGGCGCTTCCAGTACGACGGCGTCGGCCGGCTCG |
| | CCGCGATCGTCGCGCCCAACGGCGCCACCATAGCGATGGCACGCG |
| | ATGCCGCCGGGCGGCTCACCGAGCGGCGCTGGCCCGACGGCGCGA |
| | AGAGCGCCTTCGACTGGCTGCCCGAAGGCAGCCTCGCCGCCATCG |
| | AGCACAGCGCGGGCGGCAGCGCGCTCGCACAGTTCGCCTATGCCT |
| | ACGATGCCTGGGGCAACCGCACGAGCGCCACCGAGACCCTCGCGG |
| | GCACCAGCCGCAGCCTCGCCTACGGCTACGACGCGCTCGACCGCC |
| | TGAAGACCGTCACCACCGACGGTGCGACCGAAACCCATGCCTTCG |
| | ATCTCTTCGGCAATCGCACCAGCAAGACCACGGGCGGGGTGACCA |
| | CCGACTATCTCTTCGACGCGGCGCACCAGCTCACCCAGGTGCAGA |
| | TCGCCGGCACCCCCACCGAGCGGCTCGCCTACGACGACAACGGTA |
| | ATCTCCGCAAGCACTGCGTCGGCAGTCCGAGTGGCAGCACCAGCG |
| | ATTGCACCGGCACCACCGTGCTGAGCCTCGCCTGGAACGGCCTCG |
| | ACCAGTTGATCCAGGCCGCCAGGACGGGCCTGCCCGCCGAGTCCT |
| | ACGCCTACGACGATGCCGGGCGGCGTGTCACCAAGGCGGTGGGCA |
| | GCAGCGCCACCCACTTCGCCTACGACGGTCCCGACATCCTGGCCG |
| | AGTACGCCAGCCCGGCCGGCAGCCCCACCGCCGTCTATGCCCACG |
| | GTGCCGGCATCGACGAACCGCTGCTGCGCCTCACCGGCGCGACGA |
| | GCACGCCGGCCGCTTCCGCGCACCACTACGCGCAGGACGGGCTGG |
| | GCAGCATCGTCGCGGCCTATGGCGAGATCGGCGCCAGCGGTCCGG |
| | TCAGTGCCGCGAGCGTATCGGCCACCCACAGTTACAGCGCCGGCA |
| | GCTACCCGCCGGCAAAGCTGATCGACGGCGAGACGACCGGAAGC |
| | ACCGGGTTCTGGGCTGGCAGCTCGGGCAACTTCGCTGCCGATCCA |
| | GCCGTGATCACGCTGGAACTGGGTGCGGAGAAAAGCGTGAGCCGC |
| | GTGAGGCTGCACCGGGTGGCCAGCTACCTGCCCGACTACGTGGTC |
| | AAGGATGCCGAGGTGCAGGTCCGAAAACCGGACAATTCGTGGCAG |
| | ACGGTCGGCACGCTGACAAACAACACCAGCGAAGACAGTCCCGA |
| | GATCGTGCTCACCGGCGCCCCCGGCAGCGCGCTGCGCGTGCTCGT |
| | CAAGGGCGTGCGCAACGGCAGCCTGGTGCTGATGGCCGAGGTGAC |
| | GATGAGTGCGGACGGTGGCGCGGCCAGCGTGGCCACCGCCCGCTA |
| | CGACGCCTGGGGCAACGTCACGCAGGCGAGCGGCAGCATCCCGGC |
| | CTTCGGCTACACCGGACGCGAGCCCGATGCCACGGGCCTGGTCTA |
| | CTACCGCGCCCGCTACTACCACCCCGCGCTCGGCCGCTTCGCCAGC |
| | CGCGACCCGCTGGGGCTGGCGGCGGGGATCAATCCCTACGCCTAC |
| | GCGGGCGGCAATCCCATCCTCTACAACGATCCGGATGGCTTGCTG |
| | GCGCAACTGGCGTGGAATACGGCGGCCAGCTACTGGGGACAGCCG |
| | ATAGTTCAAGAAACGGTCGCCACGATTCGAAATGGGGCCGCAGTG |
| | GCCGCTGGCAACTTCGTTCCAGACACGGTCAACGGTGCAACAGGT |
| | TGGTTTGAGCAGTTCCTGCACCAAGAATCGGGCTCGTTCGGGCGC |
| | ATGGACTCGTGGGTGGATGTGCGAAACCCCGTTGCGCAGGACGTA |
| | GCCCAGGACCTGCGCGGTGTCGCAGCCGTTGGGTTAATGATGACG |
| | CCGCTGCGGTATGGTCGTGCCTCCAACGCGTCTTTCAATCCGCCAG |
| | TAGCCAATCTTCCGCTCAACACTGGAGGAAAAACATCTGGCATGT |
| | TGCACATTCCAGGGCAAGAATCACTGTCGCTCACGAGCGGAATTG |
| | CGGGGCCGTCTCAAGTCGTTAGAGGTCAAGGTTTGCCAGGATTCA |
| | ACGGTAATCAGTTGACCCATGTGGAAGGTCATGCTGCTGCTTACAT |
| | GCGGACTCACAAGGTCTCTGAGGCTGTTCTGGACATAAACAAAGC |
| | ACCTTGCACCGCTGGTAGTGGTGGTGGATGTAATGGGTTGCTTCCC |
| | CGAATGCTGCCGGAGGGGGCTCATTTAACAATTCGACACCCAAAT |
| | GGTGTTCAAGTTTATATTGGCACTCCTGACTAA [SEQ ID NO: 143] |
| Chondromyces | >AKT41505.1 type IV secretion protein Rhs [Chondromyces crocatus] |
| crocatus | MSMSASRSQPAFPFVSASSPRPRRRPPFPRALLLLIAVLLVGACGDAG |
| PROTEIN | GPLLWSSSSQALWEPSPIPPLPPLLCLGPGDGPSPFPPDLTQGTTTAAG |
| | TLPGSFSVTSTGEATYTIPVPTLPGRAGIEPSLAITYDSAQGEGLLGIGF |
| | HLQGLSSVDRCPRNVAQDGHIAPVRDAEDDALCLDGQRLVPVDPQP |
| | GRAPREYRTFPDSFTRVEADFAESEGWPAERGPKRLRAHGKAGLIYE |
| | YGGESSGRVLAQGEAVRSWLLTRLSDRDGNTMAVVYRNDLHAKGY |
| | TVEHAPQRITYTRHPTVPASRMVEFTYGPLEAADVRVHYARGMELRR |
| | SLSLRSIQMFGPGHVLARELRFGYGHGPATGRLRLEAVRECAGDGTC |
| | KPPTRFTWHTAGAAGYTQQQTLVEVPLSERGTLMTMDVSGDGLDDL |
| | VTSDMVVEAGTEEPITRWSVALNRSQELTPGFFEAAVTGQEQPHFIDA |
| | EPPYQPELGTPLDYDHDGRMDLFLHDVHGQSMTWEVLLSNGDGRFT |
| | RRDTGVPRPFTMGMTPAGLRSPDASTHLVDVDGDGMVDLLQCYLSA |
| | HEQLWYLHRWTAAAGGFAPHGDRVHALSSYPCHAELHAVDVDADG |
| | RVDLVMQELILVGSQVRAGWQYVAFSYELSDGSWTRALTGLRLTPP |
| | GDRVFFLDVNGDGLPDAVQSSRDDEQLYTSMNIGAGFAAPVPSLATP |
| | TLGAARFVRFASVLDHNADGRQDLLLAMSDGGSESLPAWKVLQATG |
| | EVGPGTFEIVHPGLPMGIVLQQDELPTPDHPLTPRVTDVNGDGAQDLL |
| | YAFNNQVHVFENVLGQEDLLAAVTDGMNAHAPEDAEYLPNVQIRYD |
| | HLIDRARTTEGFEDAPGIPSPEQRTYRPLEQSDEEPCRYPVRCVVGHR |
| | RVVSGYVLNNGADRPRTFQVAYRNGRHHRLGRGFLGFGTRIVRDLD |
| | TGAGTAEFYDNVTFDGAFQAFPFRGQVQRSWRWSPSLPLDAHSAEPA |
| | SLELLTTRSYAVVIPTQAGTYFTLSLLEGKSRHQGTFSPGSGKTLEEAV |
| | RALEGDLASRMSDTLRTVSDFDLYGNILAEQTQTEGVDLDLSVTRSF |
| | DNDPLSWRLGELTRETTCSKAGGETQCRVMHRSYDGRGHVRLERVG |
| | GEPFDPEMQLDVWFSRDALGNIHSTRSRDGTGQVRASCTSYDALGL |
| | MPYAHRNLEGHQSYTRYDPAVGVLRASVDPNGLVSRWAYDGFGRV |
| | TLESLPGRMPTVIRRTWTKDGGAAGNAWNLKIRTASVGGQDETVQL |
| | DGLGREVRWWWQALDVGEEQAPRMMQEVAFDARGEHLAWRSLPI |
| | VDPAPPGSVQVRETWQYDGMGRVLRHVTPWGAATTHEYIGRDEVIT |
| | APGQAVTRIASDPLGRPTAVGDPEGGVSRYTYGPFGGLREVTTPAGA |
| | VTLTERDAFGRVRRQVSPDRGVSTAHYDGYGQKISSLDAAGRAVTTR |
| | YDTLGRIFRQVDEDGVTEFRWDDAQHGVGQLALVVSPDGHRLRYGF |
| | DHLGRPATTTLEIGGESFTSRLSYDLSGRLERIEYPSAPGIGSFAIEREY |
| | DPHGRLRALKDAGSGAEFWRATAIDAGNRITGERFGGGTATTLRTFD |
| | AARERVSRIETQTAGGPVQQLSYLWNDRRKLVERSDGLHANVERFR |
| | YDLLDRLTCAQFGLINAALCERPFTYGPDGNLLQKPGVGAYEYDPAQ |
| | PHAVVRAGSAFYGYDAVGNQTSRPGATIAYTAFDLPKRIALTSGDTV |
| | DFAYDGLQQRVRKTTATQEIASFGEVYERVTDVVTGAVEHRYHVRN |
| | DERVVTLVRRSVAQGTRTLHVHVDHLGSIDVLTDGVTGSVAERRSY |
| | DAFGAPRHPDWGSGQPPSPHELSSLGFTGHEADLDLGLVNMKGRIYD |
| | PKLGRFLTPDPLVPRPLFGQSWNSYSYVLNSPLSLVDPSGFQEQPPATE |
| | DGCSQGCTIWVFGPPREPKPPAPPKVVEGNLEDAAGTGSTQAPVDVG |
| | TSGVRSGWSPQLPATLQTLGRGDAIARRIMDGVRIGMARMLLESAKL |
| | GILGGTSRVYVAYTNLTAAWNGYKESGLPGALDAVNPASQMVQAG |
| | VEAYEAAAAEDWEAAGASLFKAGSIGMSILATAVGVGGAITATVGST |
| | AGAAGRAAARAPSLPAYAGGKTSGVLRTTAGDTALLSGYKGPSASM |
| | PRGTPGMNGRIKSHVEAHAAAVMREQGMKEGTLYINRVPCSGATGC |
| | DAMLPRMLPPDAHLRVVGPNGYDQVFVGLPD [SEQ ID NO: 144] |
| Chondromyces | >CMC5_057130 NZ_CP012159.1: 7808731-7815414 Chondromyces |
| crocatus | crocatus strain Cm c5, complete genome |
| DNA | ATGTCCATGTCGGCCTCACGGAGTCAGCCCGCATTCCCCTTCGTGT |
| | CGGCCTCCTCTCCGCGTCCGCGCCGGCGCCCTCCCTTTCCCCGAGC |
| | GCTGCTCCTCCTCATCGCCGTGCTCCTCGTCGGCGCATGCGGCGAC |
| | GCTGGCGGCCCGCTTCTCTGGTCGAGCAGCTCCCAGGCCCTCTGGG |
| | AACCCTCCCCGATCCCGCCGCTCCCCCCGCTCCTGTGCCTCGGCCC |
| | CGGCGACGGTCCCTCCCCCTTTCCGCCTGACCTTACGCAGGGGACC |
| | ACCACCGCGGCGGGGACCCTGCCAGGGAGCTTTTCGGTCACGAGC |
| | ACGGGCGAGGCGACGTACACGATCCCGGTCCCCACGCTGCCTGGC |
| | CGTGCCGGCATCGAGCCCTCGCTGGCGATCACCTACGACAGTGCG |
| | CAGGGTGAAGGGCTGCTCGGGATCGGCTTCCACTTGCAGGGCCTC |
| | TCGTCGGTCGATCGCTGCCCCCGGAACGTCGCGCAGGATGGTCAC |
| | ATCGCGCCGGTCCGGGATGCCGAGGACGACGCCTTGTGCCTCGAT |
| | GGGCAGCGGCTCGTCCCCGTGGACCCGCAGCCAGGGCGTGCGCCG |
| | CGGGAATACCGCACGTTCCCGGACAGCTTCACGCGCGTCGAGGCC |
| | GACTTCGCGGAGAGCGAGGGGTGGCCGGCGGAGCGTGGGCCGAA |
| | GCGGCTGCGGGCGCATGGCAAAGCGGGGCTGATCTACGAATACGG |
| | TGGAGAATCATCGGGCCGGGTGCTCGCGCAAGGGGAGGCGGTGCG |
| | GTCCTGGTTGCTGACGCGGCTCAGCGACCGGGATGGCAACACGAT |
| | GGCGGTGGTCTACCGGAATGACCTCCACGCGAAGGGCTACACCGT |
| | CGAGCACGCGCCGCAGCGGATCACCTACACCAGGCACCCGACTGT |
| | GCCGGCCTCGCGCATGGTGGAGTTCACGTACGGGCCGCTGGAGGC |
| | GGCGGACGTGCGCGTACACTATGCCCGCGGGATGGAGCTGCGCCG |
| | CTCGCTGAGCTTGCGCTCGATCCAGATGTTCGGGCCGGGACACGT |
| | GCTCGCGAGGGAGCTGCGCTTCGGTTACGGGCATGGGCCGGCGAC |
| | GGGTCGCTTGCGACTGGAGGCGGTTCGGGAGTGCGCAGGTGACGG |
| | GACGTGCAAGCCGCCGACACGCTTCACCTGGCACACGGCCGGAGC |
| | GGCTGGATACACGCAGCAGCAGACACTGGTGGAGGTGCCGCTGTC |
| | GGAGCGCGGCACGTTGATGACGATGGACGTCAGCGGCGATGGCCT |
| | CGACGACCTGGTGACGTCCGACATGGTGGTGGAGGCCGGCACGGA |
| | AGAGCCGATCACCCGCTGGTCGGTCGCGCTCAACCGGAGCCAGGA |
| | GCTGACGCCGGGGTTCTTCGAGGCGGCCGTCACTGGGCAGGAGCA |
| | GCCGCATTTCATCGACGCAGAGCCGCCGTACCAGCCGGAGCTGGG |
| | GACGCCGCTCGACTACGACCACGATGGCCGGATGGACCTGTTTCT |
| | GCACGATGTGCACGGGCAGTCGATGACGTGGGAGGTGCTGCTGTC |
| | GAATGGAGATGGGCGGTTCACGCGGCGGGATACGGGGGTGCCGC |
| | GGCCGTTCACGATGGGCATGACGCCGGCGGGATTGCGCAGCCCGG |
| | ATGCGTCGACCCATCTGGTGGATGTTGACGGTGACGGGATGGTGG |
| | ACCTGCTGCAGTGCTACCTGAGCGCGCACGAGCAGCTCTGGTACTT |
| | GCACCGCTGGACGGCAGCGGCGGGGGGCTTCGCGCCGCACGGCGA |
| | TCGGGTGCATGCGCTGAGCTCCTACCCGTGCCACGCCGAGCTGCA |
| | CGCGGTCGATGTCGACGCGGATGGGCGGGTGGACCTGGTGATGCA |
| | GGAGCTGATCCTCGTCGGGAGCCAGGTGCGGGCGGGGTGGCAGTA |
| | CGTGGCGTTCTCGTACGAGCTGTCCGATGGATCGTGGACGCGCGC |
| | GCTGACGGGGCTGCGGCTCACGCCGCCTGGGGACCGGGTGTTCTT |
| | CCTCGACGTCAACGGCGATGGGCTGCCCGATGCGGTGCAGAGCAG |
| | CCGGGACGATGAGCAGCTGTACACGTCGATGAATATCGGCGCGGG |
| | ATTCGCGGCGCCGGTACCGAGCCTGGCGACGCCGACGCTCGGGGC |
| | TGCGAGGTTCGTTCGGTTTGCGTCGGTGCTCGATCACAACGCGGAT |
| | GGGCGACAAGACCTGCTGCTGGCCATGAGCGATGGGGGATCGGAG |
| | TCGCTGCCCGCGTGGAAGGTGCTCCAGGCGACGGGGGAGGTCGGT |
| | CCGGGGACGTTCGAGATCGTCCATCCCGGGCTGCCGATGGGCATC |
| | GTGCTCCAGCAGGACGAGCTGCCCACGCCCGACCATCCGCTCACG |
| | CCGCGGGTCACTGACGTGAATGGGGATGGGGCGCAGGATCTGCTC |
| | TATGCGTTCAACAACCAGGTCCATGTGTTCGAGAACGTGCTCGGCC |
| | AGGAGGACCTGCTCGCGGCCGTGACCGACGGCATGAATGCGCACG |
| | CTCCGGAGGACGCCGAGTACCTGCCCAACGTGCAGATCCGGTACG |
| | ACCACCTGATCGATCGTGCGCGGACGACGGAGGGCTTCGAGGATG |
| | CTCCAGGGATCCCGTCACCCGAGCAGCGCACCTACCGGCCTCTGG |
| | AGCAAAGCGATGAGGAGCCCTGCCGCTATCCGGTGCGGTGCGTGG |
| | TCGGGCATCGGCGGGTGGTGAGCGGCTATGTGCTCAACAATGGCG |
| | CGGATCGGCCGCGCACCTTCCAGGTGGCCTACCGCAATGGCCGTC |
| | ACCATCGCCTGGGCCGAGGGTTTCTGGGGTTCGGGACGCGGATCG |
| | TGCGTGACCTCGATACCGGCGCGGGGACGGCCGAGTTCTACGACA |
| | ACGTCACGTTTGATGGCGCCTTCCAGGCCTTCCCTTTCCGAGGGCA |
| | GGTACAGCGCTCGTGGCGCTGGAGTCCGAGCTTGCCGCTGGACGC |
| | GCATAGCGCGGAGCCGGCGTCCCTCGAGCTGCTGACGACGCGGAG |
| | CTACGCGGTGGTGATCCCCACGCAAGCGGGGACGTACTTCACCCT |
| | CTCGCTGCTGGAGGGCAAGAGCCGTCATCAGGGCACGTTCTCACC |
| | GGGGAGTGGGAAAACGCTCGAAGAAGCCGTGCGCGCTCTGGAAG |
| | GAGATCTCGCCTCGCGAATGAGCGACACGCTCCGCACCGTCAGCG |
| | ACTTCGACCTCTACGGGAACATCCTCGCCGAGCAAACGCAGACGG |
| | AGGGCGTCGACCTCGACCTCTCGGTGACGCGCAGCTTCGACAACG |
| | ACCCGCTCTCCTGGCGCCTTGGCGAGCTGACGCGAGAGACGACGT |
| | GCAGCAAAGCGGGCGGTGAGACGCAGTGCCGGGTGATGCACCGG |
| | AGCTATGACGGGCGCGGCCACGTTCGCCTGGAGCGCGTCGGGGGA |
| | GAGCCCTTCGACCCGGAGATGCAGCTCGATGTCTGGTTCTCGCGG |
| | GACGCGCTGGGCAACATCCACAGCACCCGGTCACGTGATGGGACG |
| | GGGCAGGTGCGCGCGAGCTGCACCAGCTACGACGCGCTGGGCTTG |
| | ATGCCTTATGCCCACCGCAACCTGGAGGGCCACCAGAGCTATACG |
| | CGCTACGACCCGGCCGTGGGCGTGCTGCGGGCGTCGGTGGATCCC |
| | AACGGCCTGGTGAGCCGCTGGGCCTACGATGGCTTCGGGCGGGTG |
| | ACGCTGGAGAGCCTCCCCGGGCGCATGCCCACCGTCATCCGGCGG |
| | ACCTGGACGAAGGACGGCGGAGCGGCTGGCAACGCCTGGAACCT |
| | GAAGATCCGCACCGCCTCGGTGGGGGGCCAGGACGAGACCGTGCA |
| | GCTCGATGGTCTCGGGCGGGAGGTGCGCTGGTGGTGGCAAGCGCT |
| | CGACGTGGGGGAAGAGCAAGCGCCGCGGATGATGCAGGAGGTCG |
| | CCTTCGATGCGCGGGGCGAGCACCTCGCGTGGCGCTCGCTGCCGA |
| | TCGTGGATCCCGCGCCACCAGGCTCGGTGCAGGTGCGAGAGACGT |
| | GGCAATACGACGGGATGGGGGGGGTGCTCCGGCACGTCACGCCGT |
| | GGGGGGCGGCGACGACGCACGAGTACATCGGGCGGGACGAGGTC |
| | ATCACCGCGCCTGGGCAGGCCGTCACCCGAATCGCCAGCGATCCG |
| | CTCGGGAGGCCCACGGCAGTGGGTGATCCCGAAGGTGGCGTCAGC |
| | CGGTACACCTACGGTCCCTTCGGGGGGCTGCGCGAGGTGACCACG |
| | CCCGCTGGTGCCGTGACGCTGACCGAGCGGGATGCGTTTGGCCGC |
| | GTGCGACGGCAGGTGAGCCCGGACCGGGGAGTCTCTACTGCGCAC |
| | TACGACGGTTACGGGCAGAAGATCTCATCGCTCGACGCGGCAGGA |
| | CGCGCGGTCACGACCCGCTACGACACGCTGGGTCGGATTTTCAGG |
| | CAGGTCGACGAAGACGGCGTCACCGAGTTCCGTTGGGATGACGCG |
| | CAGCATGGAGTGGGTCAGCTCGCGCTGGTGGTCAGCCCCGATGGG |
| | CATCGGCTGCGCTACGGCTTCGACCACCTCGGGCGACCAGCGACG |
| | ACGACGCTGGAGATCGGAGGGGAAAGCTTCACCAGCCGGCTGTCT |
| | TATGATCTGAGCGGCCGGCTCGAGCGGATCGAGTACCCGAGCGCG |
| | CCGGGGATTGGCAGCTTCGCCATCGAGCGGGAGTACGATCCTCAC |
| | GGGCGGCTGCGGGCGCTGAAGGATGCGGGGTCGGGGGCGGAGTT |
| | CTGGCGAGCCACCGCGATCGATGCGGGGAATCGCATCACGGGGGA |
| | GCGCTTCGGTGGGGGGACCGCCACCACGCTCCGCACGTTCGACGC |
| | GGCACGGGAGCGGGTGAGTCGGATCGAGACGCAGACGGCAGGTG |
| | GGCCCGTCCAGCAGCTCTCCTACCTCTGGAACGATCGCCGCAAGCT |
| | CGTCGAGCGCTCCGATGGCCTCCACGCCAACGTCGAGCGCTTTCGT |
| | TACGACCTGCTGGACCGGCTGACGTGCGCGCAGTTCGGGCTGATC |
| | AATGCTGCCCTCTGCGAGCGACCGTTCACCTACGGACCCGACGGC |
| | AACCTGCTCCAGAAGCCCGGCGTCGGTGCCTACGAGTACGACCCC |
| | GCGCAGCCCCACGCCGTCGTCCGAGCTGGTAGCGCGTTCTACGGC |
| | TACGACGCCGTCGGCAACCAGACCTCACGACCCGGCGCGACCATC |
| | GCCTACACCGCGTTCGACCTACCGAAGCGAATCGCGCTCACCAGC |
| | GGCGACACCGTCGACTTCGCGTACGACGGCCTCCAGCAGCGGGTG |
| | CGCAAGACCACGGCGACGCAGGAGATCGCCTCCTTCGGCGAGGTG |
| | TACGAGCGCGTGACCGATGTCGTCACGGGAGCCGTCGAGCATCGC |
| | TACCACGTGCGCAACGACGAGCGCGTCGTCACGCTGGTGCGGCGC |
| | TCGGTCGCGCAAGGCACGCGCACGCTGCATGTCCATGTCGACCAC |
| | CTCGGGTCGATCGATGTGCTCACCGACGGTGTGACCGGCAGCGTC |
| | GCCGAGCGCCGCAGCTACGATGCCTTCGGCGCACCGCGCCATCCC |
| | GACTGGGGTTCGGGTCAGCCTCCGTCACCCCACGAGCTGTCGTCGC |
| | TTGGCTTCACCGGGCACGAGGCCGACCTCGACCTCGGCCTCGTGA |
| | ACATGAAGGGGCGCATCTACGACCCCAAGCTCGGACGGTTCCTCA |
| | CGCCCGATCCGCTCGTGCCGCGGCCTCTCTTCGGGCAGAGCTGGA |
| | ATAGCTATTCGTACGTGCTAAACAGCCCGCTGTCGCTGGTCGATCC |
| | CAGTGGGTTTCAAGAGCAGCCACCTGCGACAGAGGACGGATGCTC |
| | GCAGGGCTGCACCATCTGGGTGTTCGGTCCTCCCCGCGAGCCGAA |
| | GCCACCTGCGCCGCCCAAGGTCGTCGAGGGCAACCTGGAGGACGC |
| | CGCTGGCACTGGTTCGACCCAGGCGCCGGTCGATGTCGGGACCTC |
| | CGGGGTCCGTAGCGGATGGAGTCCGCAGCTCCCGGCCACGTTGCA |
| | GACCTTGGGCCGTGGTGACGCCATCGCCAGGCGCATCATGGACGG |
| | CGTCCGCATCGGGATGGCCAGGATGCTGCTGGAGTCCGCAAAGCT |
| | CGGCATCCTGGGCGGCACCAGCCGCGTCTACGTCGCCTACACCAA |
| | CCTCACCGCCGCCTGGAATGGCTACAAAGAGAGCGGGCTCCCCGG |
| | CGCTCTCGACGCCGTCAATCCCGCCAGCCAGATGGTCCAAGCCGG |
| | CGTGGAGGCCTACGAGGCTGCCGCCGCAGAGGACTGGGAGGCCGC |
| | CGGCGCCAGCTTGTTCAAGGCCGGGTCGATCGGGATGTCGATCCT |
| | GGCGACGGCTGTTGGCGTCGGGGGAGCGATCACTGCGACAGTGGG |
| | CTCGACGGCAGGAGCGGCGGGGAGGGCAGCCGCAAGAGCCCCCT |
| | CACTCCCTGCATATGCTGGCGGAAAAACGTCGGGAGTACTACGGA |
| | CCACCGCAGGCGATACAGCACTGCTGAGCGGCTACAAGGGGCCGT |
| | CCGCATCGATGCCTCGAGGAACGCCAGGCATGAACGGACGCATCA |
| | AGTCGCATGTAGAAGCTCATGCGGCTGCCGTGATGCGAGAGCAAG |
| | GGATGAAGGAAGGAACCCTGTACATCAATCGAGTCCCCTGCTCTG |
| | GCGCCACCGGATGCGACGCGATGCTCCCAAGAATGCTCCCACCAG |
| | ATGCACACCTTCGCGTGGTCGGTCCGAATGGTTACGATCAAGTTTT |
| | TGTCGGGCTGCCCGACTGA [SEQ ID NO: 145] |

In addition, the disclosure contemplates the use any variant of any DddA amino acid sequence, including:

| | Sequence (relative to DddAtox or wildtype of | SEQ ID |
|---|---|---|
| Mutation(s) | SEQ ID NO: 338) | NO: |
| DddA | GSYALGPYQISAPQLPAYNGQTVGTFYYVNDAGGLESKV | 338 |
| (residues 1290- | FSSGGPTPYPNYANAGHVEGQSALFMRDNGISEGLVFHN | |
| 1427) or | NPEGTCGFCVNMTETLLPENAKMTVVPPEGAIPVKRGAT | |
| (DddAtox) | GETKVFTGNSNSPKSPTKGGC | |
| T1380I | GSYALGPYQISAPQLPAYNGQTVGTFYYVNDAGGLESKV | 377 |
| | FSSGGPTPYPNYANAGHVEGQSALFMRDNGISEGLVFHN | |
| | NPEGTCGFCVNMIETLLPENAKMTVVPPEGAIPVKRGAT | |
| | GETKVFTGNSNSPKSPTKGGC | |
| T1314A/T1380I | GSYALGPYQISAPQLPAYNGQTVGAFYYVNDAGGLESK | 378 |
| | VFSSGGPTPYPNYANAGHVEGQSALFMRDNGISEGLVFH | |
| | NNPEGTCGFCVNMIETLLPENAKMTVVPPEGAIPVKRGA | |
| | TGETKVFTGNSNSPKSPTKGGC | |
| Q1310R/ | GSYALGPYQISAPQLPAYNGRTVGTFYYVNDAGGLESKV | 379 |
| S1330I/ | FISGGPTPYPNYANAGHVEGQSALFMRDNGISEGLVFHN | |
| T1380I | NPEGTCGFCVNMIETLLPENAKMTVVPPEGAIPVKRGAT | |
| | GETKVFTGNSNSPKSPTKGGC | |
| T1380I/T1413I | GSYALGPYQISAPQLPAYNGQTVGTFYYVNDAGGLESKV | 380 |
| | FSSGGPTPYPNYANAGHVEGQSALFMRDNGISEGLVFHN | |
| | NPEGTCGFCVNMIETLLPENAKMTVVPPEGAIPVKRGAT | |
| | GETKVFIGNSNSPKSPTKGGC | |
| T1314A/T1380I/ | GSYALGPYQISAPQLPAYNGQTVGAFYYVNDAGGLESK | 381 |
| E1396K | VFSSGGPTPYPNYANAGHVEGQSALFMRDNGISEGLVFH | |
| | NNPEGTCGFCVNMIETLLPENAKMTVVPPKGAIPVKRGA | |
| | TGETKVFTGNSNSPKSPTKGGC | |

mitoTALEs and mitoZFs

In various embodiments, the mtDNA base editors or the polypeptides that comprise the mtDNA base editors (e.g., the pDNAbps and DddA) may include a mitoTALE as the pDNAbp component.

MitoTALEs and mitoZFP are known in the art. Each of the proteins may comprise a mitochondrial targeting sequence (MTS) in order to facilitate the translocation of the protein into the mitochondria.

In one aspect, the methods and compositions described herein involve a TALE protein programmed (e.g., engineered through manipulation of the localization signal in the C-terminus) to localize to the mitochondria (mitoTALE). In some embodiments, the localization signal comprises a sequence to target SOD2. In some embodiments, the LS comprises SEQ ID NO.: 13. In some embodiments, the LS comprises a sequence to target Cox8a. In some embodiments, the LS comprises SEQ ID NO.: 14. In some embodiments, the LS comprises a sequence with 75% or greater percent identity (e.g., 80% or greater, 85% or greater, 90% or greater, 95% or greater, 96% or greater, 97% or greater, 98% or greater, 99% or greater, 99.5% or greater, 99.9% or greater percent identity) to SEQ ID NOs.: 13 or 14.

The mitoTALE is also used to guide the fusion protein to the appropriate target nucleotide in the mtDNA. By using the RVD in the mitoTALE specific sequences can be targeted, which will place the attached DddA proximal to the target nucleotide. As used herein, “proximal” or “proximally” with respect to a target nucleotide shall mean a range of nucleic acids which are arranged consecutively upstream or downstream of the target nucleotide, on either the strand containing the target nucleotide or the strand complementary to the strand containing the target nucleotide, which when targeted and bound by a mitoTALE allow for the dimerization or re-assembly of portions of a DddA to regain, at least partially, the native activity of a full length DddA. Accordingly, the sequence should be selected from a range of nucleotides at or near the target nucleotide, or the nucleotide complementary thereto. In some embodiments, the target nucleic acid sequence is located upstream of the target nucleotide. In some embodiments, the target nucleic acid sequence is between 1 and 40 nucleotides upstream of the target nucleotide. In some embodiments, the target nucleic acid sequence is between 5 and 20 nucleotides upstream of the target nucleotide.

In some embodiments, a second mitoTALE is used. A second mitoTALE can be used to deliver additional components (e.g., additional DddA, a second portion of a DddA, additional enzymes). In some embodiments, the second mitoTALE is configured to bind a second target nucleic acid sequence. In some embodiments, the second mitoTALE is configured to bind a second target nucleic acid sequence on the nucleic acid strand complementary to the strand containing the target nucleotide. In some embodiments, the second mitoTALE is configured to bind a second target nucleic acid sequence upstream of the nucleotide complementary to the target nucleotide, which complementary nucleotide is on the nucleic acid strand complementary to the strand containing the target nucleotide. In some embodiments, the second target nucleic acid sequence is between 1 and 40 nucleotides upstream of the nucleotide complementarty to the target nucleotide, which is on the strand complementary to the strand containing the target nucleotide. In some embodiments, the second target nucleic acid sequence is between 5 and 20 nucleotides upstream of the nucleotide complementarty to the target nucleotide, which is on the strand complementary to the strand containing the target nucleotide.

In some embodiments, a mitoTALE comprises an amino acid sequence selected from any one of the following amino acid sequences, or an amino acid sequence having at least 70%, 75%, 80%, 85%, 90%, 95%, or 99% sequence identity with any one of the following mitoTALE sequences:

| SEQ ID | | |
|---|---|---|
| NO. | Sequence | Description |
| 1 | MALSRAVCGTSRQLAPVLGYLGSRQKHSLPDYPYDVPDYAGYPYDVPDY | Mito 24 |
| | AGYPYDVPDYAMDIADLRTLGYSQQQQEKIKPKVRSTVAQHHEALVGHG | |
| | FTHAHIVALSQHPAALGTVAVKYQDMIAALPEATHEAIVGVGKQWSGAR | |
| | ALEALLTVAGELRGPPLQLDTGQLLKIAKRGGVTAVEAVHAWRNALTGA | |
| | PLNLTPQQVVAIASNNGGKQALETVQRLLPVLCQAHGLTPEQVVAIASN | |
| | IGGKQALETVQALLPVLCQAHGLTPEQVVAIASHDGGKQALETVQRLLP | |
| | VLCQAHGLTPEQVVAIASHDGGKQALETVQRLLPVLCQAHGLTPEQVVA | |
| | IASHDGGKQALETVQRLLPVLCQAHGLTPEQVVAIASHDGGKQALETVQ | |
| | RLLPVLCQAHGLTPEQVVAIASHDGGKQALETVQRLLPVLCQAHGLTPE | |
| | QVVAIASNIGGKQALETVQALLPVLCQAHGLTPQQVVAIASNGGGKQAL | |
| | ETVQRLLPVLCQAHGLTPQQVVAIASNGGGRPALESIVAQLSRPDPALA | |
| | ALTNDHLVALACLGGRPALDAVKKGLGGSGSYALGPYQISAPQLPAYNG | |
| | QTVGTFYYVNDAGGLESKVFSSGGPTPYPNYANAGHVEGQSALEMRDNG | |
| | ISEGLVFHNNPEGTCGFCVNMTETLLPENAKMTVVPPEG | |
| 2 | MALSRAVCGTSRQLAPVLGYLGSRQKHSLPDYPYDVPDYAGYPYDVPDY | Mito 24a |
| | AGYPYDVPDYATNLSDIIEKETGKQLVIQESILMLPEEVEEVIGNKPES | |
| | DILVHTAYDESTDENVMLLTSDAPEYKPWALVIQDSNGENKIKMLSGGS | |
| | GGSGGSTNLSDIIEKETGKQLVIQESILMLPEEVEEVIGNKPESDILVH | |
| | TAYDESTDENVMLLTSDAPEYKPWALVIQDSNGENKIKMLSGGSMDIAD | |
| | LRTLGYSQQQQEKIKPKVRSTVAQHHEALVGHGFTHAHIVALSQHPAAL | |
| | GTVAVKYQDMIAALPEATHEAIVGVGKQWSGARALEALLTVAGELRGPP | |
| | LQLDTGQLLKIAKRGGVTAVEAVHAWRNALTGAPLNLTPQQVVAIASNN | |
| | GGKQALETVQRLLPVLCQAHGLTPEQVVAIASNIGGKQALETVQALLPV | |
| | LCQAHGLTPEQVVAIASHDGGKQALETVQRLLPVLCQAHGLTPEQVVAI | |
| | ASHDGGKQALETVQRLLPVLCQAHGLTPEQVVAIASHDGGKQALETVQR | |
| | LLPVLCQAHGLTPEQVVAIASHDGGKQALETVQRLLPVLCQAHGLTPEQ | |
| | VVAIASHDGGKQALETVQRLLPVLCQAHGLTPEQVVAIASNIGGKQALE | |
| | TVQALLPVLCQAHGLTPQQVVAIASNGGGKQALETVQRLLPVLCQAHGL | |
| | TPQQVVAIASNGGGRPALESIVAQLSRPDPALAALINDHLVALACLGGR | |
| | PALDAVKKGLGGSGSYALGPYQISAPQLPAYNGQTVGTFYYVNDAGGLE | |
| | SKVFSSGGPTPYPNYANAGHVEGQSALEMRDNGISEGLVFHNNPEGTCG | |
| | FCVNMTETLLPENAKMTVVPPEG | |
| 3 | MALSRAVCGTSRQLAPVLGYLGSRQKHSLPDYPYDVPDYAGYPYDVPDY | Mito 24b |
| | AGYPYDVPDYAMDIADLRTLGYSQQQQEKIKPKVRSTVAQHHEALVGHG | |
| | FTHAHIVALSQHPAALGTVAVKYQDMIAALPEATHEAIVGVGKQWSGAR | |
| | ALEALLTVAGELRGPPLQLDTGQLLKIAKRGGVTAVEAVHAWRNALTGA | |
| | PLNLTPQQVVAIASNNGGKQALETVQRLLPVLCQAHGLTPEQVVAIASN | |
| | IGGKQALETVQALLPVLCQAHGLTPEQVVAIASHDGGKQALETVQRLLP | |
| | VLCQAHGLTPEQVVAIASHDGGKQALETVQRLLPVLCQAHGLTPEQVVA | |
| | IASHDGGKQALETVQRLLPVLCQAHGLTPEQVVAIASHDGGKQALETVQ | |
| | RLLPVLCQAHGLTPEQVVAIASHDGGKQALETVQRLLPVLCQAHGLTPE | |
| | QVVAIASNIGGKQALETVQALLPVLCQAHGLTPQQVVAIASNGGGKQAL | |
| | ETVQRLLPVLCQAHGLTPQQVVAIASNGGGRPALESIVAQLSRPDPALA | |
| | ALTNDHLVALACLGGRPALDAVKKGLGGSGSYALGPYQISAPQLPAYNG | |
| | QTVGTFYYVNDAGGLESKVFSSGGPTPYPNYANAGHVEGQSALEMRDNG | |
| | ISEGLVFHNNPEGTCGFCVNMTETLLPENAKMTVVPPEGSGGSINLSDI | |
| | IEKETGKQLVIQESILMLPEEVEEVIGNKPESDILVHTAYDESTDENVM | |
| | LLTSDAPEYKPWALVIQDSNGENKIKMLSGGSGGSGGSTNLSDIIEKET | |
| | GKQLVIQESILMLPEEVEEVIGNKPESDILVHTAYDESTDENVMLLTSD | |
| | APEYKPWALVIQDSNGENKIKML | |
| 4 | MALSRAVCGTSRQLAPVLGYLGSRQKHSLPDYPYDVPDYAGYPYDVPDY | Mito 24c |
| | AGYPYDVPDYATNLSDIIEKETGKQLVIQESILMLPEEVEEVIGNKPES | |
| | DILVHTAYDESTDENVMLLTSDAPEYKPWALVIQDSNGENKIKMLSGGS | |
| | MDIADLRTLGYSQQQQEKIKPKVRSTVAQHHEALVGHGFTHAHIVALSQ | |
| | HPAALGTVAVKYQDMIAALPEATHEAIVGVGKQWSGARALEALLTVAGE | |
| | LRGPPLQLDTGQLLKIAKRGGVTAVEAVHAWRNALTGAPLNLTPQQVVA | |
| | IASNNGGKQALETVQRLLPVLCQAHGLTPEQVVAIASNIGGKQALETVQ | |
| | ALLPVLCQAHGLTPEQVVAIASHDGGKQALETVQRLLPVLCQAHGLTPE | |
| | QVVAIASHDGGKQALETVQRLLPVLCQAHGLTPEQVVAIASHDGGKQAL | |
| | ETVQRLLPVLCQAHGLTPEQVVAIASHDGGKQALETVQRLLPVLCQAHG | |
| | LTPEQVVAIASHDGGKQALETVQRLLPVLCQAHGLTPEQVVAIASNIGG | |
| | KQALETVQALLPVLCQAHGLTPQQVVAIASNGGGKQALETVQRLLPVLC | |
| | QAHGLTPQQVVAIASNGGGRPALESIVAQLSRPDPALAALINDHLVALA | |
| | CLGGRPALDAVKKGLGGSGSYALGPYQISAPQLPAYNGQTVGIFYYVND | |
| | AGGLESKVFSSGGPTPYPNYANAGHVEGQSALFMRDNGISEGLVFHNNP | |
| | EGTCGFCVNMTETLLPENAKMTVVPPEG | |
| 5 | MALSRAVCGTSRQLAPVLGYLGSRQKHSLPDYPYDVPDYAGYPYDVPDY | Mito 24d |
| | AGYPYDVPDYAMDIADLRTLGYSQQQQEKIKPKVRSTVAQHHEALVGHG | |
| | FTHAHIVALSQHPAALGTVAVKYQDMIAALPEATHEAIVGVGKQWSGAR | |
| | ALEALLTVAGELRGPPLQLDTGQLLKIAKRGGVTAVEAVHAWRNALTGA | |
| | PLNLTPQQVVAIASNNGGKQALETVQRLLPVLCQAHGLTPEQVVAIASN | |
| | IGGKQALETVQALLPVLCQAHGLTPEQVVAIASHDGGKQALETVQRLLP | |
| | VLCQAHGLTPEQVVAIASHDGGKQALETVQRLLPVLCQAHGLTPEQVVA | |
| | IASHDGGKQALETVQRLLPVLCQAHGLTPEQVVAIASHDGGKQALETVQ | |
| | RLLPVLCQAHGLTPEQVVAIASHDGGKQALETVQRLLPVLCQAHGLTPE | |
| | QVVAIASNIGGKQALETVQALLPVLCQAHGLTPQQVVAIASNGGGKQAL | |
| | ETVQRLLPVLCQAHGLTPQQVVAIASNGGGRPALESIVAQLSRPDPALA | |
| | ALTNDHLVALACLGGRPALDAVKKGLGGSGSYALGPYQISAPQLPAYNG | |
| | QTVGTFYYVNDAGGLESKVFSSGGPTPYPNYANAGHVEGQSALEMRDNG | |
| | ISEGLVFHNNPEGTCGFCVNMTETLLPENAKMTVVPPEGSGGSTNLSDI | |
| | IEKETGKQLVIQESILMLPEEVEEVIGNKPESDILVHTAYDESTDENVM | |
| | LLTSDAPEYKPWALVIQDSNGENKIKML | |
| 6 | MASVLTPLLLRGLTGSARRLPVPRAKIHSLDYKDHDGDYKDHDIDYKDD | Mito 28 |
| | DDKMDIADLRTLGYSQQQQEKIKPKVRSTVAQHHEALVGHGFTHAHIVA | |
| | LSQHPAALGTVAVKYQDMIAALPEATHEAIVGVGKRGAGARALEALLTV | |
| | AGELRGPPLQLDTGQLLKIAKRGGVTAVEAVHAWRNALTGAPLNLTPQQ | |
| | VVAIASNNGGKQALETVQRLLPVLCQAHGLTPEQVVAIASNIGGKQALE | |
| | TVQALLPVLCQAHGLTPQQVVAIASNGGGKQALETVQRLLPVLCQAHGL | |
| | TPQQVVAIASNNGGKQALETVQRLLPVLCQAHGLTPQQVVAIASNNGGK | |
| | QALETVQRLLPVLCQAHGLTPEQVVAIASHDGGKQALETVQRLLPVLCQ | |
| | AHGLTPQQVVAIASNGGGKQALETVQRLLPVLCQAHGLTPEQVVAIASN | |
| | IGGKQALETVQALLPVLCQAHGLTPQQVVAIASNGGGKQALETVQRLLP | |
| | VLCQAHGLTPQQVVAIASNGGGKQALETVQRLLPVLCQAHGLTPQQVVA | |
| | IASNGGGRPALESIVAQLSRPDPALAALTNDHLVALACLGGRPALDAVK | |
| | KGLGGSAIPVKRGATGETKVFTGNSNSPKSPTKGGC | |
| 7 | MASVLTPLLLRGLTGSARRLPVPRAKIHSLDYKDHDGDYKDHDIDYKDD | Mito 28a |
| | DDKTNLSDIIEKETGKQLVIQESILMLPEEVEEVIGNKPESDILVHTAY | |
| | DESTDENVMLLTSDAPEYKPWALVIQDSNGENKIKMLSGGSGGSGGSTN | |
| | LSDIIEKETGKQLVIQESILMLPEEVEEVIGNKPESDILVHTAYDESTD | |
| | ENVMLLTSDAPEYKPWALVIQDSNGENKIKMLSGGSMDIADLRTLGYSQ | |
| | QQQEKIKPKVRSTVAQHHEALVGHGFTHAHIVALSQHPAALGTVAVKYQ | |
| | DMIAALPEATHEAIVGVGKRGAGARALEALLTVAGELRGPPLQLDTGQL | |
| | LKIAKRGGVTAVEAVHAWRNALTGAPLNLTPQQVVAIASNNGGKQALET | |
| | VQRLLPVLCQAHGLTPEQVVAIASNIGGKQALETVQALLPVLCQAHGLT | |
| | PQQVVAIASNGGGKQALETVQRLLPVLCQAHGLTPQQVVAIASNNGGKQ | |
| | ALETVQRLLPVLCQAHGLTPQQVVAIASNNGGKQALETVQRLLPVLCQA | |
| | HGLTPEQVVAIASHDGGKQALETVQRLLPVLCQAHGLTPQQVVAIASNG | |
| | GGKQALETVQRLLPVLCQAHGLTPEQVVAIASNIGGKQALETVQALLPV | |
| | LCQAHGLTPQQVVAIASNGGGKQALETVQRLLPVLCQAHGLTPQQVVAI | |
| | ASNGGGKQALETVQRLLPVLCQAHGLTPQQVVAIASNGGGRPALESIVA | |
| | QLSRPDPALAALTNDHLVALACLGGRPALDAVKKGLGGSAIPVKRGATG | |
| | ETKVFTGNSNSPKSPTKGGC | |
| 8 | MASVLTPLLLRGLTGSARRLPVPRAKIHSLDYKDHDGDYKDHDIDYKDD | Mito 28b |
| | DDKMDIADLRTLGYSQQQQEKIKPKVRSTVAQHHEALVGHGFTHAHIVA | |
| | LSQHPAALGTVAVKYQDMIAALPEATHEAIVGVGKRGAGARALEALLTV | |
| | AGELRGPPLQLDTGQLLKIAKRGGVTAVEAVHAWRNALTGAPLNLTPQQ | |
| | VVAIASNNGGKQALETVQRLLPVLCQAHGLTPEQVVAIASNIGGKQALE | |
| | TVQALLPVLCQAHGLTPQQVVAIASNGGGKQALETVQRLLPVLCQAHGL | |
| | TPQQVVAIASNNGGKQALETVQRLLPVLCQAHGLTPQQVVAIASNNGGK | |
| | QALETVQRLLPVLCQAHGLTPEQVVAIASHDGGKQALETVQRLLPVLCQ | |
| | AHGLTPQQVVAIASNGGGKQALETVQRLLPVLCQAHGLTPEQVVAIASN | |
| | IGGKQALETVQALLPVLCQAHGLTPQQVVAIASNGGGKQALETVQRLLP | |
| | VLCQAHGLTPQQVVAIASNGGGKQALETVQRLLPVLCQAHGLTPQQVVA | |
| | IASNGGGRPALESIVAQLSRPDPALAALINDHLVALACLGGRPALDAVK | |
| | KGLGGSAIPVKRGATGETKVFTGNSNSPKSPTKGGCSGGSTNLSDIIEK | |
| | ETGKQLVIQESILMLPEEVEEVIGNKPESDILVHTAYDESTDENVMLLT | |
| | SDAPEYKPWALVIQDSNGENKIKMLSGGSGGSGGSTNLSDIIEKETGKQ | |
| | LVIQESILMLPEEVEEVIGNKPESDILVHTAYDESTDENVMLLTSDAPE | |
| | YKPWALVIQDSNGENKIKML | |
| 9 | MASVLTPLLLRGLTGSARRLPVPRAKIHSLDYKDHDGDYKDHDIDYKDD | Mito 28c |
| | DDKTNLSDIIEKETGKQLVIQESILMLPEEVEEVIGNKPESDILVHTAY | |
| | DESTDENVMLLISDAPEYKPWALVIQDSNGENKIKMLSGGSMDIADLRT | |
| | LGYSQQQQEKIKPKVRSTVAQHHEALVGHGFTHAHIVALSQHPAALGTV | |
| | AVKYQDMIAALPEATHEAIVGVGKRGAGARALEALLTVAGELRGPPLQL | |
| | DTGQLLKIAKRGGVTAVEAVHAWRNALTGAPLNLTPQQVVAIASNNGGK | |
| | QALETVQRLLPVLCQAHGLTPEQVVAIASNIGGKQALETVQALLPVLCQ | |
| | AHGLTPQQVVAIASNGGGKQALETVQRLLPVLCQAHGLTPQQVVAIASN | |
| | NGGKQALETVQRLLPVLCQAHGLTPQQVVAIASNNGGKQALETVQRLLP | |
| | VLCQAHGLTPEQVVAIASHDGGKQALETVQRLLPVLCQAHGLTPQQVVA | |
| | IASNGGGKQALETVQRLLPVLCQAHGLTPEQVVAIASNIGGKQALETVQ | |
| | ALLPVLCQAHGLTPQQVVAIASNGGGKQALETVQRLLPVLCQAHGLTPQ | |
| | QVVAIASNGGGKQALETVQRLLPVLCQAHGLTPQQVVAIASNGGGRPAL | |
| | ESIVAQLSRPDPALAALINDHLVALACLGGRPALDAVKKGLGGSAIPVK | |
| | RGATGETKVFTGNSNSPKSPTKGGC | |
| 10 | MASVLTPLLLRGLTGSARRLPVPRAKIHSLDYKDHDGDYKDHDIDYKDD | Mito 28d |
| | DDKMDIADLRTLGYSQQQQEKIKPKVRSTVAQHHEALVGHGFTHAHIVA | |
| | LSQHPAALGTVAVKYQDMIAALPEATHEAIVGVGKRGAGARALEALLTV | |
| | AGELRGPPLQLDTGQLLKIAKRGGVTAVEAVHAWRNALTGAPLNLTPQQ | |
| | VVAIASNNGGKQALETVQRLLPVLCQAHGLTPEQVVAIASNIGGKQALE | |
| | TVQALLPVLCQAHGLTPQQVVAIASNGGGKQALETVQRLLPVLCQAHGL | |
| | TPQQVVAIASNNGGKQALETVQRLLPVLCQAHGLTPQQVVAIASNNGGK | |
| | QALETVQRLLPVLCQAHGLTPEQVVAIASHDGGKQALETVQRLLPVLCQ | |
| | AHGLTPQQVVAIASNGGGKQALETVQRLLPVLCQAHGLTPEQVVAIASN | |
| | IGGKQALETVQALLPVLCQAHGLTPQQVVAIASNGGGKQALETVQRLLP | |
| | VLCQAHGLTPQQVVAIASNGGGKQALETVQRLLPVLCQAHGLTPQQVVA | |
| | IASNGGGRPALESIVAQLSRPDPALAALINDHLVALACLGGRPALDAVK | |
| | KGLGGSAIPVKRGATGETKVFTGNSNSPKSPTKGGCSGGSTNLSDIIEK | |
| | ETGKQLVIQESILMLPEEVEEVIGNKPESDILVHTAYDESTDENVMLLT | |
| | SDAPEYKPWALVIQDSNGENKIKML | |
| 11 | DIADLRTLGYSQQQQEKIKPKVRSTVAQHHEALVGHGFTHAHIVALSQH | Modified |
| | PAALGTVAVKYQDMIAALPEATHEAIVGVGKQWSGARALEALLTVAGEL | m.13513- |
| | RGPPLQLDTGQLLKIAKRGGVTAVEAVHAWRNALTGAPLNLTPQQVVAI | Right TALE |
| | ASHDGGKQALETVQRLLPVLCQAHGLTPEQVVAIASNGGGKQALETVQR | |
| | LLPVLCQAHGLTPQQVVAIASNGGGKQALETVQRLLPVLCQAHGLTPEQ | |
| | VVAIASNGGGKQALETVQALLPVLCQAHGLTPQQVVAIASNNGGKQALE | |
| | TVQRLLPVLCQAHGLTPQQVVAIASNNGGKQALETVQRLLPVLCQAHGL | |
| | TPEQVVAIASNIGGKQALETVQRLLPVLCQAHGLTPQQVVAIASNNGGK | |
| | QALETVQRLLPVLCQAHGLTPQQVVAIASNGGGKQALETVQRLLPVLCQ | |
| | AHGLTPEQVVAIASNIGGKQALETVQRLLPVLCQAHGLTPEQVVAIASN | |
| | NGGKQALETVQRLLPVLCQAHGLTPQQVVAIASNIGGKQALETVQRLLP | |
| | VLCQAHGLTPQQVVAIASNIGGRPALESIVAQLSRPDPALAALTNDHLV | |
| | ALACLGGRPALDAVKKGLG | |
| 12 | DIADLRTLGYSQQQQEKIKPKVRSTVAQHHEALVGHGFTHAHIVALSQH | m.8490- |
| | PAALGTVAVKYQDMIAALPEATHEAIVGVGKQWSGARALEALLTVAGEL | Right TALE |
| | RGPPLQLDTGQLLKIAKRGGVTAVEAVHAWRNALTGAPLNLTPQQVVAI | |
| | ASNIGGKQALETVQRLLPVLCQAHGLTPEQVVAIASNGGGKQALETVQR | |
| | LLPVLCQAHGLTPQQVVAIASNNGGKQALETVQRLLPVLCQAHGLTPEQ | |
| | VVAIASNNGGKQALETVQALLPVLCQAHGLTPQQVVAIASNNGGKQALE | |
| | TVQRLLPVLCQAHGLTPQQVVAIASHDGGKQALETVQRLLPVLCQAHGL | |
| | TPEQVVAIASNGGGKQALETVQRLLPVLCQAHGLTPQQVVAIASNGGGK | |
| | QALETVQRLLPVLCQAHGLTPQQVVAIASNGGGKQALETVQRLLPVLCQ | |
| | AHGLTPEQVVAIASNNGGKQALETVQRLLPVLCQAHGLTPEQVVAIASN | |
| | NGGKQALETVQRLLPVLCQAHGLTPQQVVAIASNGGGRPALESIVAQLS | |
| | RPDPALAALTNDHLVALACLGGRPALDAVKKGLG | |
| 13 | MLSRAVCGTSRQLAPVLGYLGSRQKHSLPD | SOD2 MLS |
| 14 | MSVLTPLLLRGLTGSARRLPVPRAKIHSL | COX8a MLS |
| 15 | ACCACGATCGTTATGCTGATCATACCCTAATGATCCCAGCAAGATAATG | SOD2 3′UTR |
| | TCCTGTCTTCTAAGATGTGCATCAAGCCTGGTACATACTGAAAACCCTA | |
| | TAAGGTCCTGGATAATTTTTGTTTGATTATTCATTGAAGAAACATTTAT | |
| | TTTCCAATTGTGTGAAGTTTTTGACTGTTAATAAAAGAATCTGTCAACC | |
| | ATCAAAAAAAAAAAAAAA | |
| 16 | ACCACGATCGTTATGCTGATCATACCCTAATGATCCCAGCAAGATAATG | ATP5b 3′UTR |
| | TCCTGTCTTCTAAGATGTGCATCAAGCCTGGTACATACTGAAAACCCTA | |
| | TAAGGTCCTGGATAATTTTTGTTTGATTATTCATTGAAGAAACATTTAT | |
| | TTTCCAATTGTGTGAAGTTTTTGACTGTTAATAAAAGAATCTGTCAACC | |
| | ATCAAAAAAAAAAAAAAA | |

In addition, the mitoTALE and/or mitoZFP may comprising one of the following mitochondrial targeting sequences which help promote mitochondrial localization, or an amino acid or nucleotide sequence having at least 70%, 75%, 80%, 85%, 90%, 95%, or 99% sequence identity with any one of the following sequences:

| | | |
|---|---|---|
| 13 | MLSRAVCGTSRQLAPVLGYLGSRQKHSLPD | SOD2 |
| | | (mitochondrial |
| | | superoxide |
| | | dismutase) |
| | | MTS |
| 14 | MSVLTPLLLRGLIGSARRLPVPRAKIHSL | COX8a |
| | | (mitochondrial |
| | | cytochrome |
| | | C oxidase |
| | | subunit 8A) |
| | | MTS |
| 15 | ACCACGATCGTTATGCTGATCATACCCTAATGATCCCAGCAAGATAATGTCCT | SOD2 3′UTR |
| | GTCTTCTAAGATGTGCATCAAGCCTGGTACATACTGAAAACCCTATAAGGTCC | |
| | TGGATAATTTTTGTTTGATTATTCATTGAAGAAACATTTATTTTCCAATTGTG | |
| | TGAAGTTTTTGACTGTTAATAAAAGAATCTGTCAACCATCAAAAAAAAAAAAA | |
| | AA | |
| 16 | ACCACGATCGTTATGCTGATCATACCCTAATGATCCCAGCAAGATAATGTCCT | ATP5b 3′UTR |
| | GTCTTCTAAGATGTGCATCAAGCCTGGTACATACTGAAAACCCTATAAGGTCC | |
| | TGGATAATTTTTGTTTGATTATTCATTGAAGAAACATTTATTTTCCAATTGTG | |
| | TGAAGTTTTTGACTGTTAATAAAAGAATCTGTCAACCATCAAAAAAAAAAAAA | |
| | AA | |

In various embodiments, the mtDNA base editors may comprises a mitoZF. A mitoZF may be a ZF protein comprising one or more mitochondrial localization sequences (MLS). A zinc finger is a small, functional, independently folded domain that coordinates one or more zinc ions to stabilize its structure through cysteine and/or histidine residues. Zinc fingers are structurally diverse and exhibit a wide range of functions, from DNA- or RNA-binding to protein-protein interactions and membrane association. There are more than 40 types of zinc fingers annotated in UniProtKB. The most frequent are the C21H2-type, the CCHC-type, the PHD-type and the RING-type. Examples include Accession Nos. Q7Z42, P55197, Q9P2R3, Q9P2G1, Q9P2S6, Q81UH5, P19811, Q92793, P36406, 095081, and Q9ULV3, some of which have the following sequences:

### Zinc Finger Protein: Q7Z142-1:

MPDFTIIQPD RKFDAAAVAG IFVRSSTSSS FPSASSYIAA KKRKNVDNTS TRKPYSYKDR KRKNTEEIRN IKKKLFMDLG IVRTNCGIDN EKQDREKAMK RKVTETIVTT YCELCEQNFS SSKMLLLHRG KVHNTPYIEC HLCMKLFSQT IQFNRHMKTH YGPNAKIYVQ CELCDRQFKD KQSLRTHWDV SHGSGDNQAV LA (SEQ ID NO: 414), or an amino acid having at least 70%, 75%, 80%, 85%, 90%, 95%, or 99% sequence identity therewith, or fragment thereof.

Zinc finger protein: P55197-4 (isoform-4):

MVSSDRPVSL EDEVSHSMKE MIGGCCVCSD ERGWAENPLV YCDGHGCSVA VHQACYGIVQ VPTGPWFCRK CESQERAARV RCELCPHKDG ALKRTDNGGW AHVVCALYIP EVQFANVSTM EPIVLQSVPH DRYNKTCYIC DEQGRESKAA TGACMTCNKH GCRQAFHVTC AQFAGLLCEE EGNGADNVQY CGYCKYHFSK LKKSKRGSNR SYDQSLSDSS SHSQDKHHEK EKKKYKEKDK HKQKHKKQPE PSPALVPSLT VTTEKTYTST SNNSISGSLK RLEDTTARFT NANFQEVSAH TSSGKDVSET RGSEGKGKKS SAHSSGQRGR KPGGGRNPGT TVSAASPFPQ GSFSGTPGSV KSSSGSSVQS PQDFLSFTDS DLRNDSYSHS QQSSATKDVH KGESGSQEGG VNSFSTLIGL PSTSAVTSQP KSFENSPGDL GNSSLPTAGY KRAQTSGIEE ETVKEKKRKG NKQSKHGPGR PKGNKNQENV SHLSVSSASP TSSVASAAGS ITSSSLQKSP TLLRNGSLQS LSVGSSPVGS EISMQYRHDG ACPTTTFSEL LNAIHNGIYN SNDVAVSFPN VVSGSGSSTP VSSSHLPQQS SGHLQQVGAL SPSAVSSAAP AVATTQANTL SGSSLSQAPS HMYGNRSNSS MAALIAQSEN NQTDQDLGDN SRNLVGRGSS PRGSLSPRSP VSSLQIRYDQ PGNSSLENLP PVAASIEQLL ERQWSEGQQF LLEQGTPSDI LGMLKSLHQL QVENRRLEEQ IKNLTAKKER LQLLNAQLSV PFPTITANPS PSHQIHTFSA QTAPTTDSLN SSKSPHIGNS FLPDNSLPVL NQDLTSSGQS TSSSSALSTP PPAGQSPAQQ GSGVSGVQQV NGVTVGALAS GMQPVTSTIP AVSAVGGIIG ALPGNQLAIN GIVGALNGVM QTPVTMSQNP TPLTHTTVPP NATHPMPATL TNSASGLGLL SDQQRQILIH QQQFQQLLNS QQLTPEQHQA FLYQLMQHHH QQHHQPELQQ LQIPGPTQIP INNLLAGTQA PPLHTATTNP FLTIHGDNAS QKVARLSDKT GPVAQEKS (SEQ ID NO: 415), or an amino acid having at least 70%, 75%, 80%, 85%, 90%, 95%, or 99% sequence identity therewith, or fragment thereof.

### Zinc Finger Protein: Q9P2R3-1 (Isoform 1):

MAEEEVAKLE KHLMLLRQEY VKLQKKLAET EKRCALLAAQ ANKESSSESF ISRLLAIVAD LYEQEQYSDL KIKVGDRHIS AHKFVLAARS DSWSLANLSS TKELDLSDAN PEVTMTMLRW IYTDELEFRE DDVFLTELMK LANRFQLQLL RERCEKGVMS LVNVRNCIRF YQTAEELNAS TLMNYCAEII ASHWDDLRKE DFSSMSAQLL YKMIKSKTEY PLHKAIKVER EDVVFLYLIE MDSQLPGKLN EADHNGDLAL DLALSRRLES IATTLVSHKA DVDMVDKSGW SLLHKGIQRG DLFAATFLIK NGAFVNAATL GAQETPLHLV ALYSSKKHSA DVMSEMAQIA EALLQAGANP NMQDSKGRTP LHVSIMAGNE YVFSQLLQCK QLDLELKDHE GSTALWLAVQ HITVSSDQSV NPFEDVPVVN GTSFDENSFA ARLIQRGSHT DAPDTATGNC LLQRAAGAGN EAAALFLATN GAHVNHRNKW GETPLHTACR HGLANLTAEL LQQGANPNLQ TEEALPLPKE AASLTSLADS VHLQTPLHMA IAYNHPDVVS VILEQKANAL HATNNLQIIP DFSLKDSRDQ TVLGLALWTG MHTIAAQLLG SGAAINDTMS DGQTLLHMAI QRQDSKSALF LLEHQADINV RTQDGETALQ LAIRNQLPLV VDAICTRGAD MSVPDEKGNP PLWLALANNL EDIASTLVRH GCDATCWGPG PGGCLQTLLH RAIDENNEPT ACFLIRSGCD VNSPRQPGAN GEGEEEARDG QTPLHLAASW GLEETVQCLL EFGANVNAQD AEGRTPIHVA ISSQHGVIIQ LLVSHPDIHL NVRDRQGLTP FACAMTFKNN KSAEAILKRE SGAAEQVDNK GRNFLHVAVQ NSDIESVLFL ISVHANVNSR VQDASKLTPL HLAVQAGSEI IVRNLLLAGA KVNELTKHRQ TALHLAAQQD LPTICSVLLE NGVDFAAVDE NGNNALHLAV MHGRLNNIRV LLTECTVDAE AFNLRGQSPL HILGQYGKEN AAAIFDLFLE CMPGYPLDKP DADGSTVLLL AYMKGNANLC RAIVRSGARL GVNNNQGVNI FNYQVATKQL LFRLLDMLSK EPPWCDGSYC YECTARFGVT TRKHHCRHCG RLLCHKCSTK EIPIIKFDLN KPVRVCNICF DVLTLGGVS (SEQ ID NO: 416), or an amino acid having at least 70%, 75%, 80%, 85%, 90%, 95%, or 99% sequence identity therewith, or fragment thereof.

### Zinc Finger Protein: Q9P2G1-1:

MGNTTTKFRK ALINGDENLA CQIYENNPQL KESLDPNTSY GEPYQHNTPL HYAARHGMNK ILGTFLGRDG NPNKRNVHNE TSMHLLCMGP QIMISEGALH PRLARPTEDD FRRADCLQMI LKWKGAKLDQ GEYERAAIDA VDNKKNTPLH YAAASGMKAC VELLVKHGGD LFAENENKDT PCDCAEKQHH KDLALNLESQ MVFSRDPEAE EIEAEYAALD KREPYEGLRP QDLRRLKDML IVETADMLQA PLFTAEALLR AHDWDREKLL EAWMSNPENC CQRSGVQMPT PPPSGYNAWD TLPSPRTPRT TRSSVTSPDE ISLSPGDLDT SLCDICMCSI SVFEDPVDMP CGHDFCRGCW ESFLNLKIQE GEAHNIFCPA YDCFQLVPVD IIESVVSKEM DKRYLQFDIK AFVENNPAIK WCPTPGCDRA VRLTKQGSNT SGSDTLSFPL LRAPAVDCGK GHLFCWECLG EAHEPCDCQT WKNWLQKITE MKPEELVGVS EAYEDAANCL WLLTNSKPCA NCKSPIQKNE GCNHMQCAKC KYDFCWICLE EWKKHSSSTG GYYRCTRYEV IQHVEEQSKE MTVEAEKKHK RFQELDRFMH YYTRFKNHEH SYQLEQRLLK TAKEKMEQLS RALKETEGGC PDTTFIEDAV HVLLKTRRIL KCSYPYGFFL EPKSTKKEIF ELMQTDLEMV TEDLAQKVNR PYLRTPRHKI IKAACLVQQK RQEFLASVAR GVAPADSPEA PRRSFAGGTW DWEYLGFASP EEYAEFQYRR RHRQRRRGDV HSLLSNPPDP DEPSESTLDI PEGGSSSRRP GTSVVSSASM SVLHSSSLRD YTPASRSENQ DSLQALSSLD EDDPNILLAI QLSLQESGLA LDEETRDFLS NEASLGAIGT SLPSRLDSVP RNTDSPRAAL SSSELLELGD SLMRLGAEND PFSTDTLSSH PLSEARSDFC PSSSDPDSAG QDPNINDNLL GNIMAWFHDM NPQSIALIPP ATTEISADSQ LPCIKDGSEG VKDVELVLPE DSMFEDASVS EGRGTQIEEN PLEENILAGE AASQAGDSGN EAANRGDGSD VSSQTPQTSS DWLEQVHLV (SEQ ID NO: 417), or an amino acid having at least 70%, 75%, 80%, 85%, 90%, 95%, or 99% sequence identity therewith, or fragment thereof.

### Zinc Finger Protein: Q8IUH5-1 (Isoform 1):

MGNTTTKFRK ALINGDENLA CQIYENNPQL KESLDPNTSY GEPYQHNTPL HYAARHGMNK ILGTFLGRDG NPNKRNVHNE TSMHLLCMGP QIMISEGALH PRLARPTEDD FRRADCLQMI LKWKGAKLDQ GEYERAAIDA VDNKKNTPLH YAAASGMKAC VELLVKHGGD LFAENENKDT PCDCAEKQHH KDLALNLESQ MVFSRDPEAE EIEAEYAALD KREPYEGLRP QDLRRLKDML IVETADMLQA PLFTAEALLR AHDWDREKLL EAWMSNPENC CQRSGVQMPT PPPSGYNAWD TLPSPRTPRT TRSSVTSPDE ISLSPGDLDT SLCDICMCSI SVFEDPVDMP CGHDFCRGCW ESFLNLKIQE GEAHNIFCPA YDCFQLVPVD IIESVVSKEM DKRYLQFDIK AFVENNPAIK WCPTPGCDRA VRLTKQGSNT SGSDTLSFPL LRAPAVDCGK GHLFCWECLG EAHEPCDCQT WKNWLQKITE MKPEELVGVS EAYEDAANCL WLLTNSKPCA NCKSPIQKNE GCNHMQCAKC KYDFCWICLE EWKKHSSSTG GYYRCTRYEV IQHVEEQSKE MTVEAEKKHK RFQELDRFMH YYTRFKNHEH SYQLEQRLLK TAKEKMEQLS RALKETEGGC PDTTFIEDAV HVLLKTRRIL KCSYPYGFFL EPKSTKKEIF ELMQTDLEMV TEDLAQKVNR PYLRTPRHKI IKAACLVQQK RQEFLASVAR GVAPADSPEA PRRSFAGGTW DWEYLGFASP EEYAEFQYRR RHRQRRRGDV HSLLSNPPDP DEPSESTLDI PEGGSSSRRP GTSVVSSASM SVLHSSSLRD YTPASRSENQ DSLQALSSLD EDDPNILLAI QLSLQESGLA LDEETRDFLS NEASLGAIGT SLPSRLDSVP RNTDSPRAAL SSSELLELGD SLMRLGAEND PFSTDTLSSH PLSEARSDFC PSSSDPDSAG QDPNINDNLL GNIMAWFHDM NPQSIALIPP ATTEISADSQ LPCIKDGSEG VKDVELVLPE DSMFEDASVS EGRGTQIEEN PLEENILAGE AASQAGDSGN EAANRGDGSD VSSQTPQTSS DWLEQVHLV (SEQ ID NO: 417), or an amino acid having at least 70%, 75%, 80%, 85%, 90%, 95%, or 99% sequence identity therewith, or fragment thereof.

### Zinc Finger Protein: P36406-1 (Isoform Alpha):

MATLVVNKLG AGVDSGRQGS RGTAVVKVLE CGVCEDVFSL QGDKVPRLLL CGHTVCHDCL TRLPLHGRAI RCPFDRQVTD LGDSGVWGLK KNFALLELLE RLQNGPIGQY GAAEESIGIS GESIIRCDED EAHLASVYCT VCATHLCSEC SQVTHSTKTL AKHRRVPLAD KPHEKTMCSQ HQVHAIEFVC LEEGCQTSPL MCCVCKEYGK HQGHKHSVLE PEANQIRASI LDMAHCIRTF TEEISDYSRK LVGIVQHIEG GEQIVEDGIG MAHTEHVPGT AENARSCIRA YFYDLHETLC RQEEMALSVV DAHVREKLIW LRQQQEDMTI LLSEVSAACL HCEKTLQQDD CRVVLAKQEI TRLLETLQKQ QQQFTEVADH IQLDASIPVT FTKDNRVHIG PKMEIRVVTL GLDGAGKTTI LFKLKQDEFM QPIPTIGFNV ETVEYKNLKF TIWDVGGKHK LRPLWKHYYL NTQAVVFVVD SSHRDRISEA HSELAKLLTE KELRDALLLI FANKQDVAGA LSVEEITELL SLHKLCCGRS WYIQGCDARS GMGLYEGLDW LSRQLVAAGV LDVA (SEQ ID NO: 418), or an amino acid having at least 70%, 75%, 80%, 85%, 90%, 95%, or 99% sequence identity therewith, or fragment thereof.

### Zinc Finger Protein: Q9ULV3-1 (Isoform-1):

MFSQQQQQQL QQQQQQLQQL QQQQLQQQQL QQQQLLQLQQ LLQQSPPQAP LPMAVSRGLP PQQPQQPLLN LQGTNSASLL NGSMLQRALL LQQLQGLDQF AMPPATYDTA GLTMPTATLG NLRGYGMASP GLAAPSLTPP QLATPNLQQF FPQATRQSLL GPPPVGVPMN PSQFNLSGRN PQKQARTSSS TTPNRKDSSS QTMPVEDKSD PPEGSEEAAE PRMDTPEDQD LPPCPEDIAK EKRTPAPEPE PCEASELPAK RLRSSEEPTE KEPPGQLQVK AQPQARMTVP KQTQTPDLLP EALEAQVLPR FQPRVLQVQA QVQSQTQPRI PSTDTQVQPK LQKQAQTQTS PEHLVLQQKQ VQPQLQQEAE PQKQVQPQVQ PQAHSQGPRQ VQLQQEAEPL KQVQPQVQPQ AHSQPPRQVQ LQLQKQVQTQ TYPQVHTQAQ PSVQPQEHPP AQVSVQPPEQ THEQPHTQPQ VSLLAPEQTP VVVHVCGLEM PPDAVEAGGG MEKTLPEPVG TQVSMEEIQN ESACGLDVGE CENRAREMPG VWGAGGSLKV TILQSSDSRA FSTVPLTPVP RPSDSVSSTP AATSTPSKQA LQFFCYICKA SCSSQQEFQD HMSEPQHQQR LGEIQHMSQA CLLSLLPVPR DVLETEDEEP PPRRWCNTCQ LYYMGDLIQH RRTQDHKIAK QSLRPFCTVC NRYFKTPRKF VEHVKSQGHK DKAKELKSLE KEIAGQDEDH FITVDAVGCF EGDEEEEEDD EDEEEIEVEE ELCKQVRSRD ISREEWKGSE TYSPNTAYGV DFLVPVMGYI CRICHKFYHS NSGAQLSHCK SLGHFENLQK YKAAKNPSPT TRPVSRRCAI NARNALTALF TSSGRPPSQP NTQDKTPSKV TARPSQPPLP RRSTRLKT (SEQ ID NO: 419), or an amino acid having at least 70%, 75%, 80%, 85%, 90%, 95%, or 99% sequence identity therewith, or fragment thereof.

The present disclosure may use any known or available zinc finger protein, or variant or functional fragment thereof. In some embodiments, a mitoZF comprises an amino acid sequence selected from any one of the following amino acid sequences, or an amino acid sequence having at least 70%, 75%, 80%, 85%, 90%, 95%, or 99% sequence identity with any one of the following mitoZF sequences:

ZF (R8)
(SEQ ID NO: 398)
MAERPFQCDICMRNFSTSGSLSRHIRTHTGEKPFQCDICMRNFSQSGSLT
RHIRTHTGSEKPFQCDICMRNFSRSDALSQHIRTHTGEKPFQCDICMRNF
SRNDNRITHIRTHTGEKPFQCDICMRNFSRSDHLTQHTKIHLR
ZF (5xZnF-4-R8)
(SEQ ID NO: 402)
MAERPFQCDICMRNFSQASNLISHIRTHTGEKPFQCDICMRNFSTSHSLT
EHIRTHTGSEKPFQCDICMRNFSERSHLREHIRTHTGEKPFQCDICMRNF
SQSGNLTEHIRTHTGEKPFQCDICMRNESSKKALTEHTKIHLR
ZF (5xZnF-10-R8)
(SEQ ID NO: 403)
MAERPFQCDICMRNFSQASNLISHIRTHTGEKPFQCDICMRNFSQRANLR
AHIRTHTGSEKPFQCDICMRNFSQASNLISHIRTHTGEKPFQCDICMRNF
STSHSLTEHIRTHTGEKPFQCDICMRNFSERSHLREHTKIHLR
ZF (R13-1)
(SEQ ID NO: 404)
MAERPFQCDICMRNFSRSDNLSTHIRTHTGEKPFQCDICMRNFSDRSDLS
RHIRTHTGEKPFQCDICMRNFSQSGDLTRHIRTHTGSEKPFQCDICMRNF
SRSDSLSAHIRTHTGEKPFQCDICMRNFSQKATRITHTKIHLR
ZF (5xZnF-9-R13)
(SEQ ID NO: 405)
MAERPFQCDICMRNFSQSSSLVRHIRTHTGEKPFQCDICMRNFSRSDNLV
RHIRTHTGSEKPFQCDICMRNFSQAGHLASHIRTHTGEKPFQCDICMRNF
SRKDNLKNHIRTHTGEKPFQCDICMRNFSRKDALRGHTKIHLR
ZF (5xZnF-12-R13)
(SEQ ID NO: 406)
MAERPFQCDICMRNFSRSDHLTTHIRTHTGEKPFQCDICMRNFSQSSSLV
RHIRTHTGSEKPFQCDICMRNFSRSDNLVRHIRTHTGEKPFQCDICMRNF
SQAGHLASHIRTHTGEKPFQCDICMRNFSRKDNLKNHTKIHLR

napDNAbp

In various embodiments, the mtDNA base editors or the polypeptides that comprise the mtDNA base editors (e.g., the pDNAbps and DddA) may include a napDNAbp as the pDNAbp component.

In one aspect, the methods and base editor compositions described herein involve a nucleic acid programmable DNA binding protein (napDNAbp). Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence. In various embodiments, the napDNAbp can be fused to a herein disclosed adenosine deaminase or cytidine deaminase.

Without being bound by theory, the binding mechanism of a napDNAbp—guide RNA complex, in general, includes the step of forming an R-loop whereby the napDNAbp induces the unwinding of a double-strand DNA target, thereby separating the strands in the region bound by the napDNAbp. The guideRNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”).

The below description of various napDNAbps which can be used in connection with the presently disclose base editors is not meant to be limiting in any way. The base editors may comprise the canonical SpCas9, or any ortholog Cas9 protein, or any variant Cas9 protein-including any naturally occurring variant, mutant, or otherwise engineered version of Cas9 that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also may also contain various modifications that alter/enhance their PAM specifities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequence or a reference Cas9 equivalent (e.g., Cas12a/Cpf1).

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the base editor (PE) of the invention.

As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

Examples of Cas9 and Cas9 equivalents are provided as follows; however, these specific examples are not meant to be limiting. The base editor fusions of the present disclosure may use any suitable napDNAbp, including any suitable Cas9 or Cas9 equivalent.

### (1) Wild Type SpCas9

In one embodiment, the base editor constructs described herein may comprise the “canonical SpCas9” nuclease from S. pyogenes, which has been widely used as a tool for genome engineering. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9 or variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the following amino acid sequence:

| Description | Sequence | SEQ ID NO: |
|---|---|---|
| SpCas9 | MDKKYSIGLDIGTNSVGWAVITDEYKVPSKKFKVLGNTDRHSIKKNLIGALLEDS | 28 |
| Streptococcus | GETAEATRLKRTARRRYTRRKNRICYLQEIFSNEMAKVDDSFFHRLEESELVEED | |
| pyogenes M1 | KKHERHPIFGNIVDEVAYHEKYPTIYHLRKKLVDSTDKADLRLIYLALAHMIKER | |
| SwissProt | GHFLIEGDLNPDNSDVDKLFIQLVQTYNQLFEENPINASGVDAKAILSARLSKSR | |
| Accession | RLENLIAQLPGEKKNGLFGNLIALSLGLIPNEKSNEDLAEDAKLQLSKDTYDDDL | |
| No. Q99ZW2 | DNLLAQIGDQYADLFLAAKNLSDAILLSDILRVNTEITKAPLSASMIKRYDEHHQ | |
| Wild type | DLTLLKALVRQQLPEKYKEIFFDQSKNGYAGYIDGGASQEEFYKFIKPILEKMDG | |
| | TEELLVKLNREDLLRKQRTFDNGSIPHQIHLGELHAILRRQEDFYPFLKDNREKI | |
| | EKILTFRIPYYVGPLARGNSRFAWMTRKSEETITPWNFEEVVDKGASAQSFIERM | |
| | TNFDKNLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKIIKDKD | |
| | FLDNEENEDILEDIVLILTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSG | |
| | QGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVD | |
| | QELDINRLSDYDVDHIVPQSFLKDDSIDNKVLTRSDKNRGKSDNVPSEEVVKKMK | |
| | NYWRQLLNAKLITQRKFDNLTKAERGGLSELDKAGFIKRQLVETRQITKHVAQIL | |
| | DSRMNTKYDENDKLIREVKVITLKSKLVSDFRKDFQFYKVREINNYHHAHDAYLN | |
| | AVVGTALIKKYPKLESEFVYGDYKVYDVRKMIAKSEQEIGKATAKYFFYSNIMNE | |
| | FKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSK | |
| | KLKSVKELLGITIMERSSFEKNPIDFLEAKGYKEVKKDLIIKLPKYSLFELENGR | |
| | KRMLASAGELQKGNELALPSKYVNFLYLASHYEKLKGSPEDNEQKQLFVEQHKHY | |
| | LDEIIEQISEFSKRVILADANLDKVLSAYNKHRDKPIREQAENIIHLFTLINLGA | |
| | PAAFKYFDTTIDRKRYTSTKEVLDATLIHQSITGLYETRIDLSQLGGD | |
| SpCas9 | ATGGATAAAAAATATAGCATTGGCCTGGATATTGGCACCAACAGCGTGGGCTGGG | 29 |
| Reverse | CGGTGATTACCGATGAATATAAAGTGCCGAGCAAAAAATTTAAAGTGCTGGGCAA | |
| translation | CACCGATCGCCATAGCATTAAAAAAAACCTGATTGGCGCGCTGCTGTTTGATAGC | |
| of | GGCGAAACCGCGGAAGCGACCCGCCTGAAACGCACCGCGCGCCGCCGCTATACCC | |
| SwissProt | GCCGCAAAAACCGCATTTGCTATCTGCAGGAAATTTTTAGCAACGAAATGGCGAA | |
| Accession | AGTGGATGATAGCTTTTTTCATCGCCTGGAAGAAAGCTTTCTGGTGGAAGAAGAT | |
| No. Q99ZW2 | AAAAAACATGAACGCCATCCGATTTTTGGCAACATTGTGGATGAAGTGGCGTATC | |
| Streptococcus | ATGAAAAATATCCGACCATTTATCATCTGCGCAAAAAACTGGTGGATAGCACCGA | |
| pyogenes | TAAAGCGGATCTGCGCCTGATTTATCTGGCGCTGGCGCATATGATTAAATTTCGC | |
| | GGCCATTTTCTGATTGAAGGCGATCTGAACCCGGATAACAGCGATGTGGATAAAC | |
| | TGTTTATTCAGCTGGTGCAGACCTATAACCAGCTGTTTGAAGAAAACCCGATTAA | |
| | CGCGAGCGGCGTGGATGCGAAAGCGATTCTGAGCGCGCGCCTGAGCAAAAGCCGC | |
| | CGCCTGGAAAACCTGATTGCGCAGCTGCCGGGCGAAAAAAAAAACGGCCTGTTTG | |
| | GCAACCTGATTGCGCTGAGCCTGGGCCTGACCCCGAACTTTAAAAGCAACTTTGA | |
| | TCTGGCGGAAGATGCGAAACTGCAGCTGAGCAAAGATACCTATGATGATGATCTG | |
| | GATAACCTGCTGGCGCAGATTGGCGATCAGTATGCGGATCTGTTTCTGGCGGCGA | |
| | AAAACCTGAGCGATGCGATTCTGCTGAGCGATATTCTGCGCGTGAACACCGAAAT | |
| | TACCAAAGCGCCGCTGAGCGCGAGCATGATTAAACGCTATGATGAACATCATCAG | |
| | GATCTGACCCTGCTGAAAGCGCTGGTGCGCCAGCAGCTGCCGGAAAAATATAAAG | |
| | AAATTTTTTTTGATCAGAGCAAAAACGGCTATGCGGGCTATATTGATGGCGGCGC | |
| | GAGCCAGGAAGAATTTTATAAATTTATTAAACCGATTCTGGAAAAAATGGATGGC | |
| | ACCGAAGAACTGCTGGTGAAACTGAACCGCGAAGATCTGCTGCGCAAACAGCGCA | |
| | CCTTTGATAACGGCAGCATTCCGCATCAGATTCATCTGGGCGAACTGCATGCGAT | |
| | TCTGCGCCGCCAGGAAGATTTTTATCCGTTTCTGAAAGATAACCGCGAAAAAATT | |
| | GAAAAAATTCTGACCTTTCGCATTCCGTATTATGTGGGCCCGCTGGCGCGCGGCA | |
| | ACAGCCGCTTTGCGTGGATGACCCGCAAAAGCGAAGAAACCATTACCCCGTGGAA | |
| | CTTTGAAGAAGTGGTGGATAAAGGCGCGAGCGCGCAGAGCTTTATTGAACGCATG | |
| | ACCAACTTTGATAAAAACCTGCCGAACGAAAAAGTGCTGCCGAAACATAGCCTGC | |
| | TGTATGAATATTTTACCGTGTATAACGAACTGACCAAAGTGAAATATGTGACCGA | |
| | AGGCATGCGCAAACCGGCGTTTCTGAGCGGCGAACAGAAAAAAGCGATTGTGGAT | |
| | CTGCTGTTTAAAACCAACCGCAAAGTGACCGTGAAACAGCTGAAAGAAGATTATT | |
| | TTAAAAAAATTGAATGCTTTGATAGCGTGGAAATTAGCGGCGTGGAAGATCGCTT | |
| | TAACGCGAGCCTGGGCACCTATCATGATCTGCTGAAAATTATTAAAGATAAAGAT | |
| | TTTCTGGATAACGAAGAAAACGAAGATATTCTGGAAGATATTGTGCTGACCCTGA | |
| | CCCTGITTGAAGATCGCGAAATGATTGAAGAACGCCTGAAAACCTATGCGCATCT | |
| | GTTTGATGATAAAGTGATGAAACAGCTGAAACGCCGCCGCTATACCGGCTGGGGC | |
| | CGCCTGAGCCGCAAACTGATTAACGGCATTCGCGATAAACAGAGCGGCAAAACCA | |
| | TTCTGGATTTTCTGAAAAGCGATGGCTTTGCGAACCGCAACTTTATGCAGCTGAT | |
| | TCATGATGATAGCCTGACCTTTAAAGAAGATATTCAGAAAGCGCAGGTGAGCGGC | |
| | CAGGGCGATAGCCTGCATGAACATATTGCGAACCTGGCGGGCAGCCCGGCGATTA | |
| | AAAAAGGCATTCTGCAGACCGTGAAAGTGGTGGATGAACTGGTGAAAGTGATGGG | |
| | CCGCCATAAACCGGAAAACATTGTGATTGAAATGGCGCGCGAAAACCAGACCACC | |
| | CAGAAAGGCCAGAAAAACAGCCGCGAACGCATGAAACGCATTGAAGAAGGCATTA | |
| | AAGAACTGGGCAGCCAGATTCTGAAAGAACATCCGGTGGAAAACACCCAGCTGCA | |
| | GAACGAAAAACTGTATCTGTATTATCTGCAGAACGGCCGCGATATGTATGTGGAT | |
| | CAGGAACTGGATATTAACCGCCTGAGCGATTATGATGTGGATCATATTGTGCCGC | |
| | AGAGCTTTCTGAAAGATGATAGCATTGATAACAAAGTGCTGACCCGCAGCGATAA | |
| | AAACCGCGGCAAAAGCGATAACGTGCCGAGCGAAGAAGTGGTGAAAAAAATGAAA | |
| | AACTATTGGCGCCAGCTGCTGAACGCGAAACTGATTACCCAGCGCAAATTTGATA | |
| | ACCTGACCAAAGCGGAACGCGGCGGCCTGAGCGAACTGGATAAAGCGGGCTTTAT | |
| | TAAACGCCAGCTGGTGGAAACCCGCCAGATTACCAAACATGTGGCGCAGATTCTG | |
| | GATAGCCGCATGAACACCAAATATGATGAAAACGATAAACTGATTCGCGAAGTGA | |
| | AAGTGATTACCCTGAAAAGCAAACTGGTGAGCGATTTTCGCAAAGATTTTCAGIT | |
| | TTATAAAGTGCGCGAAATTAACAACTATCATCATGCGCATGATGCGTATCTGAAC | |
| | GCGGTGGTGGGCACCGCGCTGATTAAAAAATATCCGAAACTGGAAAGCGAATTTG | |
| | TGTATGGCGATTATAAAGTGTATGATGTGCGCAAAATGATTGCGAAAAGCGAACA | |
| | GGAAATTGGCAAAGCGACCGCGAAATATTTTTTTTATAGCAACATTATGAACTTT | |
| | TTTAAAACCGAAATTACCCTGGCGAACGGCGAAATTCGCAAACGCCCGCTGATTG | |
| | AAACCAACGGCGAAACCGGCGAAATTGTGTGGGATAAAGGCCGCGATTTTGCGAC | |
| | CGTGCGCAAAGTGCTGAGCATGCCGCAGGTGAACATTGTGAAAAAAACCGAAGTG | |
| | CAGACCGGCGGCTTTAGCAAAGAAAGCATTCTGCCGAAACGCAACAGCGATAAAC | |
| | TGATTGCGCGCAAAAAAGATTGGGATCCGAAAAAATATGGCGGCTTTGATAGCCC | |
| | GACCGTGGCGTATAGCGTGCTGGTGGTGGCGAAAGTGGAAAAAGGCAAAAGCAAA | |
| | AAACTGAAAAGCGTGAAAGAACTGCTGGGCATTACCATTATGGAACGCAGCAGCT | |
| | TTGAAAAAAACCCGATTGATTTTCTGGAAGCGAAAGGCTATAAAGAAGTGAAAAA | |
| | AGATCTGATTATTAAACTGCCGAAATATAGCCTGTTTGAACTGGAAAACGGCCGC | |
| | AAACGCATGCTGGCGAGCGCGGGCGAACTGCAGAAAGGCAACGAACTGGCGCTGC | |
| | CGAGCAAATATGTGAACTTTCTGTATCTGGCGAGCCATTATGAAAAACTGAAAGG | |
| | CAGCCCGGAAGATAACGAACAGAAACAGCTGTTTGTGGAACAGCATAAACATTAT | |
| | CTGGATGAAATTATTGAACAGATTAGCGAATTTAGCAAACGCGTGATTCTGGCGG | |
| | ATGCGAACCTGGATAAAGTGCTGAGCGCGTATAACAAACATCGCGATAAACCGAT | |
| | TCGCGAACAGGCGGAAAACATTATTCATCTGTTTACCCTGACCAACCTGGGCGCG | |
| | CCGGCGGCGTTTAAATATTTTGATACCACCATTGATCGCAAACGCTATACCAGCA | |
| | CCAAAGAAGTGCTGGATGCGACCCTGATTCATCAGAGCATTACCGGCCTGTATGA | |
| | AACCCGCATTGATCTGAGCCAGCTGGGCGGCGAT | |

The base editors described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 entry, which include:

| SpCas9 mutation (relative | Function/Characteristic (as reported) |
|---|---|
| to the amino acid sequence | (see UniProtKB - Q99ZW2 |
| of the canonical SpCas9 | (CAS9_STRPT1) entry - incorporated |
| sequence, SEQ ID NO: 28) | herein by reference) |
| D10A | Nickase mutant which cleaves the |
| | protospacer strand (but no cleavage |
| | of non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased DNA cleavage |
| 312-409 deletion | No nuclease activity |
| E762A | Nickase |
| H840A | Nickase mutant which cleaves the |
| | non-protospacer strand but does |
| | not cleave the protospacer strand |
| N854A | Nickase |
| N863A | Nickase |
| H982A | Decreased DNA cleavage |
| D986A | Nickase |
| 1099-1368 deletion | No nuclease activity |
| R1333A | Reduced DNA binding |

Other wild type SpCas9 sequences that may be used in the present disclosure, include:

| Description | Sequence | SEQ ID NO: |
|---|---|---|
| SpCas9 | ATGGATAAGAAATACTCAATAGGCTTAGATATCGGCACAAATAGCGTCGGATGGGCGGT | 30 |
| Streptococcus | GATCACTGATGATTATAAGGTTCCGTCTAAAAAGTTCAAGGTTCTGGGAAATACAGACC | |
| pyogenes | GCCACAGTATCAAAAAAAATCTTATAGGGGCTCTTTTATTTGGCAGTGGAGAGACAGCG | |
| MGAS1882 | GAAGCGACTCGTCTCAAACGGACAGCTCGTAGAAGGTATACACGTCGGAAGAATCGTAT | |
| wild type | TTGTTATCTACAGGAGATTTTTTCAAATGAGATGGCGAAAGTAGATGATAGTTTCTTTC | |
| NC_017053.1 | ATCGACTTGAAGAGTCTTTTTTGGTGGAAGAAGACAAGAAGCATGAACGTCATCCTATT | |
| | TTTGGAAATATAGTAGATGAAGTTGCTTATCATGAGAAATATCCAACTATCTATCATCT | |
| | GCGAAAAAAATTGGCAGATTCTACTGATAAAGCGGATTTGCGCTTAATCTATTTGGCCT | |
| | TAGCGCATATGATTAAGTTTCGTGGTCATTTTTTGATTGAGGGAGATTTAAATCCTGAT | |
| | AATAGTGATGTGGACAAACTATTTATCCAGTTGGTACAAATCTACAATCAATTATTTGA | |
| | AGAAAACCCTATTAACGCAAGTAGAGTAGATGCTAAAGCGATTCTTTCTGCACGATTGA | |
| | GTAAATCAAGACGATTAGAAAATCTCATTGCTCAGCTCCCCGGTGAGAAGAGAAATGGC | |
| | TTGTTTGGGAATCTCATTGCTTTGTCATTGGGATTGACCCCTAATTTTAAATCAAATTT | |
| | TGATTTGGCAGAAGATGCTAAATTACAGCTTTCAAAAGATACTTACGATGATGATTTAG | |
| | ATAATTTATTGGCGCAAATTGGAGATCAATATGCTGATTTGTTTTTGGCAGCTAAGAAT | |
| | TTATCAGATGCTATTTTACTTTCAGATATCCTAAGAGTAAATAGTGAAATAACTAAGGC | |
| | TCCCCTATCAGCTTCAATGATTAAGCGCTACGATGAACATCATCAAGACTTGACTCTTT | |
| | TAAAAGCTTTAGTTCGACAACAACTTCCAGAAAAGTATAAAGAAATCTTTTTTGATCAA | |
| | TCAAAAAACGGATATGCAGGTTATATTGATGGGGGAGCTAGCCAAGAAGAATTTTATAA | |
| | ATTTATCAAACCAATTTTAGAAAAAATGGATGGTACTGAGGAATTATTGGTGAAACTAA | |
| | ATCGTGAAGATTTGCTGCGCAAGCAACGGACCTTTGACAACGGCTCTATTCCCCATCAA | |
| | ATTCACTTGGGTGAGCTGCATGCTATTTTGAGAAGACAAGAAGACTTTTATCCATTTTT | |
| | AAAAGACAATCGTGAGAAGATTGAAAAAATCTTGACTTTTCGAATTCCTTATTATGTTG | |
| | GTCCATTGGCGCGTGGCAATAGTCGTTTTGCATGGATGACTCGGAAGTCTGAAGAAACA | |
| | ATTACCCCATGGAATTTTGAAGAAGTTGTCGATAAAGGTGCTTCAGCTCAATCATTTAT | |
| | TGAACGCATGACAAACTTTGATAAAAATCTTCCAAATGAAAAAGTACTACCAAAACATA | |
| | GTTTGCTTTATGAGTATTTTACGGITTATAACGAATTGACAAAGGTCAAATATGTTACT | |
| | GAGGGAATGCGAAAACCAGCATTTCTTTCAGGTGAACAGAAGAAAGCCATTGTTGATTT | |
| | ACTCTTCAAAACAAATCGAAAAGTAACCGTTAAGCAATTAAAAGAAGATTATTTCAAAA | |
| | AAATAGAATGTTTTGATAGTGTTGAAATTTCAGGAGTTGAAGATAGATTTAATGCTTCA | |
| | TTAGGCGCCTACCATGATTTGCTAAAAATTATTAAAGATAAAGATTTTTTGGATAATGA | |
| | AGAAAATGAAGATATCTTAGAGGATATTGTTTTAACATTGACCTTATTTGAAGATAGGG | |
| | GGATGATTGAGGAAAGACTTAAAACATATGCTCACCTCTTTGATGATAAGGTGATGAAA | |
| | CAGCTTAAACGTCGCCGTTATACTGGTTGGGGACGTTTGTCTCGAAAATTGATTAATGG | |
| | TATTAGGGATAAGCAATCTGGCAAAACAATATTAGATTTTTTGAAATCAGATGGTTTTG | |
| | CCAATCGCAATTTTATGCAGCTGATCCATGATGATAGTTTGACATTTAAAGAAGATATT | |
| | CAAAAAGCACAGGTGTCTGGACAAGGCCATAGTTTACATGAACAGATTGCTAACTTAGC | |
| | TGGCAGTCCTGCTATTAAAAAAGGTATTTTACAGACTGTAAAAATTGTTGATGAACTGG | |
| | TCAAAGTAATGGGGCATAAGCCAGAAAATATCGTTATTGAAATGGCACGTGAAAATCAG | |
| | ACAACTCAAAAGGGCCAGAAAAATTCGCGAGAGCGTATGAAACGAATCGAAGAAGGTAT | |
| | CAAAGAATTAGGAAGTCAGATTCTTAAAGAGCATCCTGTTGAAAATACTCAATTGCAAA | |
| | ATGAAAAGCTCTATCTCTATTATCTACAAAATGGAAGAGACATGTATGTGGACCAAGAA | |
| | TTAGATATTAATCGTTTAAGTGATTATGATGTCGATCACATTGTTCCACAAAGTTTCAT | |
| | TAAAGACGATTCAATAGACAATAAGGTACTAACGCGTTCTGATAAAAATCGTGGTAAAT | |
| | CGGATAACGTTCCAAGTGAAGAAGTAGTCAAAAAGATGAAAAACTATTGGAGACAACTT | |
| | CTAAACGCCAAGTTAATCACTCAACGTAAGTTTGATAATTTAACGAAAGCTGAACGTGG | |
| | AGGTTTGAGTGAACTTGATAAAGCTGGTTTTATCAAACGCCAATTGGTTGAAACTCGCC | |
| | AAATCACTAAGCATGTGGCACAAATTTTGGATAGTCGCATGAATACTAAATACGATGAA | |
| | AATGATAAACTTATTCGAGAGGTTAAAGTGATTACCTTAAAATCTAAATTAGTTTCTGA | |
| | CTTCCGAAAAGATTTCCAATTCTATAAAGTACGTGAGATTAACAATTACCATCATGCCC | |
| | ATGATGCGTATCTAAATGCCGTCGTTGGAACTGCTTTGATTAAGAAATATCCAAAACTT | |
| | GAATCGGAGTTTGTCTATGGTGATTATAAAGTTTATGATGTTCGTAAAATGATTGCTAA | |
| | GTCTGAGCAAGAAATAGGCAAAGCAACCGCAAAATATTTCTTTTACTCTAATATCATGA | |
| | ACTTCTTCAAAACAGAAATTACACTTGCAAATGGAGAGATTCGCAAACGCCCTCTAATC | |
| | GAAACTAATGGGGAAACTGGAGAAATTGTCTGGGATAAAGGGCGAGATTTTGCCACAGT | |
| | GCGCAAAGTATTGTCCATGCCCCAAGTCAATATTGTCAAGAAAACAGAAGTACAGACAG | |
| | GCGGATTCTCCAAGGAGTCAATTTTACCAAAAAGAAATTCGGACAAGCTTATTGCTCGT | |
| | AAAAAAGACTGGGATCCAAAAAAATATGGTGGTTTTGATAGTCCAACGGTAGCTTATTC | |
| | AGTCCTAGTGGTTGCTAAGGTGGAAAAAGGGAAATCGAAGAAGTTAAAATCCGTTAAAG | |
| | AGTTACTAGGGATCACAATTATGGAAAGAAGTTCCTTTGAAAAAAATCCGATTGACTTT | |
| | TTAGAAGCTAAAGGATATAAGGAAGTTAAAAAAGACTTAATCATTAAACTACCTAAATA | |
| | TAGTCTTTTTGAGTTAGAAAACGGTCGTAAACGGATGCTGGCTAGTGCCGGAGAATTAC | |
| | AAAAAGGAAATGAGCTGGCTCTGCCAAGCAAATATGTGAATTTTTTATATTTAGCTAGT | |
| | CATTATGAAAAGTTGAAGGGTAGTCCAGAAGATAACGAACAAAAACAATTGTTTGTGGA | |
| | GCAGCATAAGCATTATTTAGATGAGATTATTGAGCAAATCAGTGAATTTTCTAAGCGTG | |
| | TTATTTTAGCAGATGCCAATTTAGATAAAGTTCTTAGTGCATATAACAAACATAGAGAC | |
| | AAACCAATACGTGAACAAGCAGAAAATATTATTCATTTATTTACGTTGACGAATCTTGG | |
| | AGCTCCCGCTGCTTTTAAATATTTTGATACAACAATTGATCGTAAACGATATACGTCTA | |
| | CAAAAGAAGTTTTAGATGCCACTCTTATCCATCAATCCATCACTGGTCTTTATGAAACA | |
| | CGCATTGATTTGAGTCAGCTAGGAGGTGACTGA | |
| SpCas9 | MDKKYSIGLDIGTNSVGWAVITDDYKVPSKKFKVLGNTDRHSIKKNLIGALLFGSGETA | 31 |
| Streptococcus | EATRLKRTARRRYTRRKNRICYLQEIFSNEMAKVDDSFFHRLEESELVEEDKKHERHPI | |
| pyogenes | FGNIVDEVAYHEKYPTIYHLRKKLADSTDKADLRLIYLALAHMIKERGHFLIEGDLNPD | |
| MGAS1882 | NSDVDKLFIQLVQIYNQLFEENPINASRVDAKAILSARLSKSRRLENLIAQLPGEKRNG | |
| wild type | LFGNLIALSLGLTPNFKSNFDLAEDAKLILSKDTYDDDLDNLLAQIGDQYADLFLAAKN | |
| NC_017053.1 | LSDAILLSDILRVNSEITKAPLSASMIKRYDEHHQDLILLKALVRIILPEKYKEIFFDQ | |
| | SKNGYAGYIDGGASQEEFYKFIKPILEKMDGTEELLVKLNREDLLRKIRTEDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKILKEDYFKKIECFDSVEISGVEDRENAS | |
| | LGAYHDLLKIIKDKDELDNEENEDILEDIVLTLTLFEDRGMIEERLKTYAHLEDDKVMK | |
| | QLKRRRYTGWGRLSRKLINGIRDKQSGKTILDFLKSDGFANRNFMQLIHDDSLTFKEDI | |
| | QKAQVSGQGHSLHEQIANLAGSPAIKKGILQTVKIVDELVKVMGHKPENIVIEMARENQ | |
| | TTQKGQKNSRERMKRIEEGIKELGSQILKEHPVENTQLQNEKLYLYYLINGRDMYVDQE | |
| | LDINRLSDYDVDHIVPQSFIKDDSIDNKVLTRSDKNRGKSDNVPSEEVVKKMKNYWRQL | |
| | LNAKLITQRKFDNLTKAERGGLSELDKAGFIKRQLVETRIITKHVAQILDSRMNTKYDE | |
| | NDKLIREVKVITLKSKLVSDFRKDFQFYKVREINNYHHAHDAYLNAVVGTALIKKYPKL | |
| | ESEFVYGDYKVYDVRKMIAKSEQEIGKATAKYFFYSNIMNFFKTEITLANGEIRKRPLI | |
| | ETNGETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKNPIDF | |
| | LEAKGYKEVKKDLIIKLPKYSLFELENGRKRMLASAGELQKGNELALPSKYVNFLYLAS | |
| | HYEKLKGSPEDNEQKILFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |
| SpCas9 | ATGGATAAAAAGTATTCTATTGGTTTAGACATCGGCACTAATTCCGTTGGATGGGCTGT | 32 |
| Streptococcus | CATAACCGATGAATACAAAGTACCTTCAAAGAAATTTAAGGTGTTGGGGAACACAGACC | |
| pyogenes | GTCATTCGATTAAAAAGAATCTTATCGGTGCCCTCCTATTCGATAGTGGCGAAACGGCA | |
| wild type | GAGGCGACTCGCCTGAAACGAACCGCTCGGAGAAGGTATACACGTCGCAAGAACCGAAT | |
| SWBC2D7W014 | ATGTTACTTACAAGAAATTTTTAGCAATGAGATGGCCAAAGTTGACGATTCTTTCTTTC | |
| | ACCGTTTGGAAGAGTCCTTCCTTGTCGAAGAGGACAAGAAACATGAACGGCACCCCATC | |
| | TTTGGAAACATAGTAGATGAGGTGGCATATCATGAAAAGTACCCAACGATTTATCACCT | |
| | CAGAAAAAAGCTAGTTGACTCAACTGATAAAGCGGACCTGAGGTTAATCTACTTGGCTC | |
| | TTGCCCATATGATAAAGTTCCGTGGGCACTTTCTCATTGAGGGTGATCTAAATCCGGAC | |
| | AACTCGGATGTCGACAAACTGTTCATCCAGTTAGTACAAACCTATAATCAGTTGITTGA | |
| | AGAGAACCCTATAAATGCAAGTGGCGTGGATGCGAAGGCTATTCTTAGCGCCCGCCTCT | |
| | CTAAATCCCGACGGCTAGAAAACCTGATCGCACAATTACCCGGAGAGAAGAAAAATGGG | |
| | TTGTTCGGTAACCTTATAGCGCTCTCACTAGGCCTGACACCAAATTTTAAGTCGAACTT | |
| | CGACTTAGCTGAAGATGCCAAATTGCAGCTTAGTAAGGACACGTACGATGACGATCTCG | |
| | ACAATCTACTGGCACAAATTGGAGATCAGTATGCGGACTTATTTTTGGCTGCCAAAAAC | |
| | CTTAGCGATGCAATCCTCCTATCTGACATACTGAGAGTTAATACTGAGATTACCAAGGC | |
| | GCCGTTATCCGCTTCAATGATCAAAAGGTACGATGAACATCACCAAGACTTGACACTTC | |
| | TCAAGGCCCTAGTCCGTCAGCAACTGCCTGAGAAATATAAGGAAATATTCTTTGATCAG | |
| | TCGAAAAACGGGTACGCAGGTTATATTGACGGCGGAGCGAGTCAAGAGGAATTCTACAA | |
| | GTTTATCAAACCCATATTAGAGAAGATGGATGGGACGGAAGAGTTGCTTGTAAAACTCA | |
| | ATCGCGAAGATCTACTGCGAAAGCAGCGGACTTTCGACAACGGTAGCATTCCACATCAA | |
| | ATCCACTTAGGCGAATTGCATGCTATACTTAGAAGGCAGGAGGATTTTTATCCGTTCCT | |
| | CAAAGACAATCGTGAAAAGATTGAGAAAATCCTAACCTTTCGCATACCTTACTATGTGG | |
| | GACCCCTGGCCCGAGGGAACTCTCGGTTCGCATGGATGACAAGAAAGTCCGAAGAAACG | |
| | ATTACTCCATGGAATTTTGAGGAAGTTGTCGATAAAGGTGCGTCAGCTCAATCGTTCAT | |
| | CGAGAGGATGACCAACTTTGACAAGAATTTACCGAACGAAAAAGTATTGCCTAAGCACA | |
| | GTTTACTTTACGAGTATTTCACAGTGTACAATGAACTCACGAAAGITAAGTATGTCACT | |
| | GAGGGCATGCGTAAACCCGCCTTTCTAAGCGGAGAACAGAAGAAAGCAATAGTAGATCT | |
| | GTTATTCAAGACCAACCGCAAAGTGACAGTTAAGCAATTGAAAGAGGACTACTTTAAGA | |
| | AAATTGAATGCTTCGATTCTGTCGAGATCTCCGGGGTAGAAGATCGATTTAATGCGTCA | |
| | CTTGGTACGTATCATGACCTCCTAAAGATAATTAAAGATAAGGACTTCCTGGATAACGA | |
| | AGAGAATGAAGATATCTTAGAAGATATAGTGTTGACTCTTACCCTCTTTGAAGATCGGG | |
| | AAATGATTGAGGAAAGACTAAAAACATACGCTCACCTGTTCGACGATAAGGTTATGAAA | |
| | CAGTTAAAGAGGCGTCGCTATACGGGCTGGGGACGATTGTCGCGGAAACTTATCAACGG | |
| | GATAAGAGACAAGCAAAGTGGTAAAACTATTCTCGATTTTCTAAAGAGCGACGGCTTCG | |
| | CCAATAGGAACTTTATGCAGCTGATCCATGATGACTCITTAACCTTCAAAGAGGATATA | |
| | CAAAAGGCACAGGTTTCCGGACAAGGGGACTCATTGCACGAACATATTGCGAATCTTGC | |
| | TGGTTCGCCAGCCATCAAAAAGGGCATACTCCAGACAGTCAAAGTAGTGGATGAGCTAG | |
| | TTAAGGTCATGGGACGTCACAAACCGGAAAACATTGTAATCGAGATGGCACGCGAAAAT | |
| | CAAACGACTCAGAAGGGGCAAAAAAACAGTCGAGAGCGGATGAAGAGAATAGAAGAGGG | |
| | TATTAAAGAACTGGGCAGCCAGATCTTAAAGGAGCATCCTGTGGAAAATACCCAATTGC | |
| | AGAACGAGAAACTTTACCTCTATTACCTACAAAATGGAAGGGACATGTATGTTGATCAG | |
| | GAACTGGACATAAACCGTTTATCTGATTACGACGTCGATCACATTGTACCCCAATCCTT | |
| | TTTGAAGGACGATTCAATCGACAATAAAGTGCTTACACGCTCGGATAAGAACCGAGGGA | |
| | AAAGTGACAATGTTCCAAGCGAGGAAGTCGTAAAGAAAATGAAGAACTATTGGCGGCAG | |
| | CTCCTAAATGCGAAACTGATAACGCAAAGAAAGTTCGATAACTTAACTAAAGCTGAGAG | |
| | GGGTGGCTTGTCTGAACTTGACAAGGCCGGATTTATTAAACGTCAGCTCGTGGAAACCC | |
| | GCCAAATCACAAAGCATGTTGCACAGATACTAGATTCCCGAATGAATACGAAATACGAC | |
| | GAGAACGATAAGCTGATTCGGGAAGTCAAAGTAATCACTTTAAAGTCAAAATTGGTGTC | |
| | GGACTTCAGAAAGGATTTTCAATTCTATAAAGTTAGGGAGATAAATAACTACCACCATG | |
| | CGCACGACGCTTATCITAATGCCGTCGTAGGGACCGCACTCATTAAGAAATACCCGAAG | |
| | CTAGAAAGTGAGTTTGTGTATGGTGATTACAAAGTTTATGACGTCCGTAAGATGATCGC | |
| | GAAAAGCGAACAGGAGATAGGCAAGGCTACAGCCAAATACTTCTTTTATTCTAACATTA | |
| | TGAATTTCTTTAAGACGGAAATCACTCTGGCAAACGGAGAGATACGCAAACGACCTTTA | |
| | ATTGAAACCAATGGGGAGACAGGTGAAATCGTATGGGATAAGGGCCGGGACTTCGCGAC | |
| | GGTGAGAAAAGTTTTGTCCATGCCCCAAGTCAACATAGTAAAGAAAACTGAGGTGCAGA | |
| | CCGGAGGGTTTTCAAAGGAATCGATTCTTCCAAAAAGGAATAGTGATAAGCTCATCGCT | |
| | CGTAAAAAGGACTGGGACCCGAAAAAGTACGGTGGCTTCGATAGCCCTACAGTTGCCTA | |
| | TTCTGTCCTAGTAGTGGCAAAAGTTGAGAAGGGAAAATCCAAGAAACTGAAGTCAGTCA | |
| | AAGAATTATTGGGGATAACGATTATGGAGCGCTCGTCTTTTGAAAAGAACCCCATCGAC | |
| | TTCCTTGAGGCGAAAGGTTACAAGGAAGTAAAAAAGGATCTCATAATTAAACTACCAAA | |
| | GTATAGTCTGTTTGAGTTAGAAAATGGCCGAAAACGGATGTTGGCTAGCGCCGGAGAGC | |
| | TTCAAAAGGGGAACGAACTCGCACTACCGTCTAAATACGTGAATTTCCTGTATTTAGCG | |
| | TCCCATTACGAGAAGTTGAAAGGTTCACCTGAAGATAACGAACAGAAGCAACTTTTTGT | |
| | TGAGCAGCACAAACATTATCTCGACGAAATCATAGAGCAAATTTCGGAATTCAGTAAGA | |
| | GAGTCATCCTAGCTGATGCCAATCTGGACAAAGTATTAAGCGCATACAACAAGCACAGG | |
| | GATAAACCCATACGTGAGCAGGCGGAAAATATTATCCATTTGTTTACTCTTACCAACCT | |
| | CGGCGCTCCAGCCGCATTCAAGTATTTTGACACAACGATAGATCGCAAACGATACACTT | |
| | CTACCAAGGAGGTGCTAGACGCGACACTGATTCACCAATCCATCACGGGATTATATGAA | |
| | ACTCGGATAGATTTGTCACAGCTTGGGGGTGACGGATCCCCCAAGAAGAAGAGGAAAGT | |
| | CTCGAGCGACTACAAAGACCATGACGGTGATTATAAAGATCATGACATCGATTACAAGG | |
| | ATGACGATGACAAGGCTGCAGGA | |
| SpCas9 | MDKKYSIGLDIGTNSVGWAVITDEYKVPSKKFKVLGNTDRHSIKKNLIGALLFDSGETA | 33 |
| Streptococcus | EATRLKRTARRRYTRRKNRICYLQEIFSNEMAKVDDSFFHRLEESELVEEDKKHERHPI | |
| pyogenes | FGNIVDEVAYHEKYPTIYHLRKKLVDSTDKADLRLIYLALAHMIKFRGHFLIEGDLNPD | |
| wild type | NSDVDKLFIQLVQTYNQLFEENPINASGVDAKAILSARLSKSRRLENLIAILPGEKKNG | |
| Encoded | LFGNLIALSLGLIPNFKSNEDLAEDAKLILSKDTYDDDLDNLLAQIGDQYADLFLAAKN | |
| product of | LSDAILLSDILRVNTEITKAPLSASMIKRYDEHHQDLILLKALVRQQLPEKYKEIFFDQ | |
| SWBC2D7W014 | SKNGYAGYIDGGASQEEFYKFIKPILEKMDGTEELLVKLNREDLLRKIRTEDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKILKEDYFKKIECFDSVEISGVEDRENAS | |
| | LGTYHDLLKIIKDKDELDNEENEDILEDIVLILTLFEDREMIEERLKTYAHLEDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLINGRDMYVDQ | |
| | ELDINRLSDYDVDHIVPQSFLKDDSIDNKVLTRSDKNRGKSDNVPSEEVVKKMKNYWRQ | |
| | LLNAKLITIRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGDGSPKKKRKVSSDYKDHDGDYKDHDIDYKDDDDKAAG | |
| SpCas9 | ATGGATAAGAAATACTCAATAGGCTTAGATATCGGCACAAATAGCGTCGGATGGGCGGT | 34 |
| Streptococcus | GATCACTGATGAATATAAGGTTCCGTCTAAAAAGTTCAAGGTTCTGGGAAATACAGACC | |
| pyogenes | GCCACAGTATCAAAAAAAATCTTATAGGGGCTCTTTTATTTGACAGTGGAGAGACAGCG | |
| M1GAS wild | GAAGCGACTCGTCTCAAACGGACAGCTCGTAGAAGGTATACACGTCGGAAGAATCGTAT | |
| type | TTGTTATCTACAGGAGATTTTTTCAAATGAGATGGCGAAAGTAGATGATAGTTTCTTTC | |
| NC_002737.2 | ATCGACTTGAAGAGTCTTTTTTGGTGGAAGAAGACAAGAAGCATGAACGTCATCCTATT | |
| | TTTGGAAATATAGTAGATGAAGTTGCTTATCATGAGAAATATCCAACTATCTATCATCT | |
| | GCGAAAAAAATTGGTAGATTCTACTGATAAAGCGGATTTGCGCTTAATCTATTTGGCCT | |
| | TAGCGCATATGATTAAGTTTCGTGGTCATTTTTTGATTGAGGGAGATTTAAATCCTGAT | |
| | AATAGTGATGTGGACAAACTATTTATCCAGTTGGTACAAACCTACAATCAATTATTTGA | |
| | AGAAAACCCTATTAACGCAAGTGGAGTAGATGCTAAAGCGATTCITTCTGCACGATTGA | |
| | GTAAATCAAGACGATTAGAAAATCTCATTGCTCAGCTCCCCGGTGAGAAGAAAAATGGC | |
| | TTATTTGGGAATCTCATTGCTTTGTCATTGGGTTTGACCCCTAATTTTAAATCAAATTT | |
| | TGATTTGGCAGAAGATGCTAAATTACAGCTTTCAAAAGATACTTACGATGATGATTTAG | |
| | ATAATTTATTGGCGCAAATTGGAGATCAATATGCTGATTTGTTTTTGGCAGCTAAGAAT | |
| | TTATCAGATGCTATTTTACTTTCAGATATCCTAAGAGTAAATACTGAAATAACTAAGGC | |
| | TCCCCTATCAGCTTCAATGATTAAACGCTACGATGAACATCATCAAGACTTGACTCTTT | |
| | TAAAAGCTTTAGTTCGACAACAACTTCCAGAAAAGTATAAAGAAATCTTTTTTGATCAA | |
| | TCAAAAAACGGATATGCAGGTTATATTGATGGGGGAGCTAGCCAAGAAGAATTTTATAA | |
| | ATTTATCAAACCAATTTTAGAAAAAATGGATGGTACTGAGGAATTATTGGTGAAACTAA | |
| | ATCGTGAAGATTTGCTGCGCAAGCAACGGACCTTTGACAACGGCTCTATTCCCCATCAA | |
| | ATTCACTTGGGTGAGCTGCATGCTATTTTGAGAAGACAAGAAGACTTTTATCCATTTTT | |
| | AAAAGACAATCGTGAGAAGATTGAAAAAATCTTGACTTTTCGAATTCCTTATTATGTTG | |
| | GTCCATTGGCGCGTGGCAATAGTCGTTTTGCATGGATGACTCGGAAGTCTGAAGAAACA | |
| | ATTACCCCATGGAATTTTGAAGAAGTTGTCGATAAAGGTGCTTCAGCTCAATCATTTAT | |
| | TGAACGCATGACAAACTTTGATAAAAATCTTCCAAATGAAAAAGTACTACCAAAACATA | |
| | GTTTGCTTTATGAGTATTTTACGGTTTATAACGAATTGACAAAGGTCAAATATGTTACT | |
| | GAAGGAATGCGAAAACCAGCATTTCTTTCAGGTGAACAGAAGAAAGCCATTGTTGATTT | |
| | ACTCTTCAAAACAAATCGAAAAGTAACCGTTAAGCAATTAAAAGAAGATTATTTCAAAA | |
| | AAATAGAATGTTTTGATAGTGTTGAAATTTCAGGAGTTGAAGATAGATTTAATGCTTCA | |
| | TTAGGTACCTACCATGATTTGCTAAAAATTATTAAAGATAAAGATTTTTTGGATAATGA | |
| | AGAAAATGAAGATATCTTAGAGGATATTGTTTTAACATTGACCTTATTTGAAGATAGGG | |
| | AGATGATTGAGGAAAGACTTAAAACATATGCTCACCTCTTTGATGATAAGGTGATGAAA | |
| | CAGCTTAAACGTCGCCGTTATACTGGTTGGGGACGTTTGTCTCGAAAATTGATTAATGG | |
| | TATTAGGGATAAGCAATCTGGCAAAACAATATTAGATTTTTTGAAATCAGATGGTTTTG | |
| | CCAATCGCAATTTTATGCAGCTGATCCATGATGATAGTTTGACATTTAAAGAAGACATT | |
| | CAAAAAGCACAAGTGTCTGGACAAGGCGATAGTTTACATGAACATATTGCAAATTTAGC | |
| | TGGTAGCCCTGCTATTAAAAAAGGTATTTTACAGACTGTAAAAGTTGTTGATGAATTGG | |
| | TCAAAGTAATGGGGCGGCATAAGCCAGAAAATATCGTTATTGAAATGGCACGTGAAAAT | |
| | CAGACAACTCAAAAGGGCCAGAAAAATTCGCGAGAGCGTATGAAACGAATCGAAGAAGG | |
| | TATCAAAGAATTAGGAAGTCAGATTCTTAAAGAGCATCCTGTTGAAAATACTCAATTGC | |
| | AAAATGAAAAGCTCTATCTCTATTATCTCCAAAATGGAAGAGACATGTATGTGGACCAA | |
| | GAATTAGATATTAATCGTTTAAGTGATTATGATGTCGATCACATTGTTCCACAAAGTTT | |
| | CCTTAAAGACGATTCAATAGACAATAAGGTCTTAACGCGTTCTGATAAAAATCGTGGTA | |
| | AATCGGATAACGTTCCAAGTGAAGAAGTAGTCAAAAAGATGAAAAACTATTGGAGACAA | |
| | CTTCTAAACGCCAAGTTAATCACTCAACGTAAGITTGATAATTTAACGAAAGCTGAACG | |
| | TGGAGGTTTGAGTGAACTTGATAAAGCTGGTTTTATCAAACGCCAATTGGTTGAAACTC | |
| | GCCAAATCACTAAGCATGTGGCACAAATTTTGGATAGTCGCATGAATACTAAATACGAT | |
| | GAAAATGATAAACTTATTCGAGAGGTTAAAGTGATTACCTTAAAATCTAAATTAGTTTC | |
| | TGACTTCCGAAAAGATTTCCAATTCTATAAAGTACGTGAGATTAACAATTACCATCATG | |
| | CCCATGATGCGTATCTAAATGCCGTCGTTGGAACTGCTTTGATTAAGAAATATCCAAAA | |
| | CTTGAATCGGAGTTTGTCTATGGTGATTATAAAGTTTATGATGTTCGTAAAATGATTGC | |
| | TAAGTCTGAGCAAGAAATAGGCAAAGCAACCGCAAAATATTTCTTTTACTCTAATATCA | |
| | TGAACTTCTTCAAAACAGAAATTACACTTGCAAATGGAGAGATTCGCAAACGCCCTCTA | |
| | ATCGAAACTAATGGGGAAACTGGAGAAATTGTCTGGGATAAAGGGCGAGATTTTGCCAC | |
| | AGTGCGCAAAGTATTGTCCATGCCCCAAGTCAATATTGTCAAGAAAACAGAAGTACAGA | |
| | CAGGCGGATTCTCCAAGGAGTCAATTTTACCAAAAAGAAATTCGGACAAGCTTATTGCT | |
| | CGTAAAAAAGACTGGGATCCAAAAAAATATGGTGGTTTTGATAGTCCAACGGTAGCTTA | |
| | TTCAGTCCTAGTGGTTGCTAAGGTGGAAAAAGGGAAATCGAAGAAGTTAAAATCCGTTA | |
| | AAGAGTTACTAGGGATCACAATTATGGAAAGAAGTTCCTTTGAAAAAAATCCGATTGAC | |
| | TTTTTAGAAGCTAAAGGATATAAGGAAGTTAAAAAAGACTTAATCATTAAACTACCTAA | |
| | ATATAGTCTTTTTGAGTTAGAAAACGGTCGTAAACGGATGCTGGCTAGTGCCGGAGAAT | |
| | TACAAAAAGGAAATGAGCTGGCTCTGCCAAGCAAATATGTGAATTTTTTATATTTAGCT | |
| | AGTCATTATGAAAAGTTGAAGGGTAGTCCAGAAGATAACGAACAAAAACAATTGTTTGT | |
| | GGAGCAGCATAAGCATTATTTAGATGAGATTATTGAGCAAATCAGTGAATTTTCTAAGC | |
| | GTGTTATTTTAGCAGATGCCAATTTAGATAAAGTTCTTAGTGCATATAACAAACATAGA | |
| | GACAAACCAATACGTGAACAAGCAGAAAATATTATTCATTTATTTACGTTGACGAATCT | |
| | TGGAGCTCCCGCTGCTTTTAAATATTTTGATACAACAATTGATCGTAAACGATATACGT | |
| | CTACAAAAGAAGTTTTAGATGCCACTCTTATCCATCAATCCATCACTGGTCTTTATGAA | |
| | ACACGCATTGATTTGAGTCAGCTAGGAGGTGACTGA | |
| SpCas9 | MDKKYSIGLDIGTNSVGWAVITDEYKVPSKKFKVLGNTDRHSIKKNLIGALLFDSGETA | 35 |
| Streptococcus | EATRLKRTARRRYTRRKNRICYLQEIFSNEMAKVDDSFFHRLEESELVEEDKKHERHPI | |
| pyogenes | FGNIVDEVAYHEKYPTIYHLRKKLVDSTDKADLRLIYLALAHMIKFRGHFLIEGDLNPD | |
| M1GAS wild | NSDVDKLFIQLVQTYNQLFEENPINASGVDAKAILSARLSKSRRLENLIAQLPGEKKNG | |
| type | LFGNLIALSLGLTPNFKSNFDLAEDAKLQLSKDTYDDDLDNLLAQIGDQYADLFLAAKN | |
| Encoded | LSDAILLSDILRVNTEITKAPLSASMIKRYDEHHQDLILLKALVRIQLPEKYKEIFFDQ | |
| product of | SKNGYAGYIDGGASQEEFYKFIKPILEKMDGTEELLVKLNREDLLRKIRTEDNGSIPHQ | |
| NC_002737.2 | IHLGELHAILRRQEDFYPFLKDNREKIEKILTFRIPYYVGPLARGNSRFAWMTRKSEET | |
| (100% | ITPWNFEEVVDKGASAQSFIERMTNFDKNLPNEKVLPKHSLLYEYFTVYNELTKVKYVT | |
| identical to | EGMRKPAFLSGEQKKAIVDLLFKTNRKVTVKILKEDYFKKIECFDSVEISGVEDRENAS | |
| the canonical | LGTYHDLLKIIKDKDELDNEENEDILEDIVLTLTLFEDREMIEERLKTYAHLEDDKVMK | |
| Q99ZW2 | QLKRRRYTGWGRLSRKLINGIRDKQSGKTILDFLKSDGFANRNFMQLIHDDSLTFKEDI | |
| wild type) | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLINGRDMYVDQ | |
| | ELDINRLSDYDVDHIVPQSFLKDDSIDNKVLTRSDKNRGKSDNVPSEEVVKKMKNYWRQ | |
| | LLNAKLITIRKFDNLTKAERGGLSELDKAGFIKRQLVETRIITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |

The base editors described herein may include any of the above SpCas9 sequences, or any variant thereof having at least 80%, at least 85%, at least 90%, at least 95%, or at least 99% sequence identity thereto.

### (2) Wild Type Cas9 Orthologs

In other embodiments, the Cas9 protein can be a wild type Cas9 ortholog from another bacterial species. For example, the following Cas9 orthologs can be used in connection with the base editor constructs described in this specification. In addition, any variant Cas9 orthologs having at least 80%, at least 85%, at least 90%, at least 95%, or at least 99% sequence identity to any of the below orthologs may also be used with the present base editors.

| Description | Sequence |
|---|---|
| LfCas9 | MKEYHIGLDIGTSSIGWAVTDSQFKLMRIKGKTAIGVRLFEEGKTAAERRTFRTTRRRLKRRKWRLHYLDEIFAPHLQEVD |
| Lactobacillus | ENFLRRLKQSNIHPEDPTKNQAFIGKLLFPDLLKKNERGYPTLIKMRDELPVEQRAHYPVMNIYKLREAMINEDRQFDLRE |
| fermentum | VYLAVHHIVKYRGHFLNNASVDKFKVGRIDFDKSFNVLNEAYEELQNGEGSFTIEPSKVEKIGQLLLDTKMRKLDRQKAVA |
| wild type | KLLEVKVADKEETKRNKQIATAMSKLVLGYKADFATVAMANGNEWKIDLSSETSEDEIEKFREELSDAQNDILTEITSLFS |
| GenBank: | QIMLNEIVPNGMSISESMMDRYWTHERQLAEVKEYLATQPASARKEFDQVYNKYIGQAPKERGFDLEKGLKKILSKKENWK |
| SNX31424.11 | EIDELLKAGDFLPKQRTSANGVIPHQMHQQELDRIIEKQAKYYPWLATENPATGERDRHQAKYELDQLVSFRIPYYVGPLV |
| | TPEVQKATSGAKFAWAKRKEDGEITPWNLWDKIDRAESAEAFIKRMTVKDTYLLNEDVLPANSLLYQKYNVLNELNNVRVN |
| | GRRLSVGIKQDIYTELFKKKKTVKASDVASLVMAKTRGVNKPSVEGLSDPKKFNSNLATYLDLKSIVGDKVDDNRYQTDLE |
| | NIIEWRSVFEDGEIFADKLTEVEWLTDEQRSALVKKRYKGWGRLSKKLLTGIVDENGQRIIDLMWNTDQNFKEIVDQPVFK |
| | EQIDQLNQKAITNDGMTLRERVESVLDDAYTSPQNKKAIWQVVRVVEDIVKAVGNAPKSISIEFARNEGNKGEITRSRRTQ |
| | LQKLFEDQAHELVKDTSLTEELEKAPDLSDRYYFYFTQGGKDMYTGDPINFDEISTKYDIDHILPQSFVKDNSLDNRVLTS |
| | RKENNKKSDQVPAKLYAAKMKPYWNQLLKQGLITQRKFENLTKDVDQNIKYRSLGFVKRQLVETRQVIKLTANILGSMYQE |
| | AGTEIIETRAGLTKQLREEFDLPKVREVNDYHHAVDAYLTTFAGQYLNRRYPKLRSFFVYGEYMKFKHGSDLKLRNFNFFH |
| | ELMEGDKSQGKVVDQQTGELITTRDEVAKSFDRLLNMKYMLVSKEVHDRSDQLYGATIVTAKESGKLTSPIEIKKNRLVDL |
| | YGAYTNGTSAFMTIIKFTGNKPKYKVIGIPTTSAASLKRAGKPGSESYNQELHRIIKSNPKVKKGFEIVVPHVSYGQLIVD |
| | GDCKFTLASPTVQHPATQLVLSKKSLETISSGYKILKDKPAIANERLIRVFDEVVGQMNRYFTIFDQRSNRQKVADARDKF |
| | LSLPTESKYEGAKKVQVGKTEVITNLLMGLHANATQGDLKVLGLATFGFFQSTTGLSLSEDTMIVYQSPTGLFERRICLKD |
| | I (SEQ ID NO: 36) |
| SaCas9 | MDKKYSIGLDIGTNSVGWAVITDEYKVPSKKFKVLGNTDRHSIKKNLIGALLFDSGETAEATRLKRTARRRYTRRKNRICY |
| Staphylococcus | LQEIFSNEMAKVDDSFFHRLEESFLVEEDKKHERHPIFGNIVDEVAYHEKYPTIYHLRKKLVDSTDKADLRLIYLALAHMI |
| aureus | KFRGHFLIEGDLNPDNSDVDKLFIQLVQTYNQLFEENPINASGVDAKAILSARLSKSRRLENLIAQLPGEKKNGLFGNLIA |
| wild type | LSLGLTPNFKSNFDLAEDAKLQLSKDTYDDDLDNLLAQIGDQYADLFLAAKNLSDAILLSDILRVNTEITKAPLSASMIKR |
| GenBank: | YDEHHQDLTLLKALVRQQLPEKYKEIFFDQSKNGYAGYIDGGASQEEFYKFIKPILEKMDGTEELLVKLNREDLLRKQRTF |
| AYD60528.1 | DNGSIPHQIHLGELHAILRRQEDFYPFLKDNREKIEKILTFRIPYYVGPLARGNSRFAWMTRKSEETITPWNFEEVVDKGA |
| | SAQSFIERMTNFDKNLPNEKVLPKHSLLYEYFTVYNELTKVKYVTEGMRKPAFLSGEQKKAIVDLLFKTNRKVTVKQLKED |
| | YFKKIECFDSVEISGVEDRFNASLGTYHDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDSLTFKEDIQKAQVSGQGDSLHEHIANLAG |
| | SPAIKKGILQTVKVVDELVKVMGRHKPENIVIEMARENQTTQKGQKNSRERMKRIEEGIKELGSQILKEHPVENTQLQNEK |
| | LYLYYLQNGRDMYVDQELDINRLSDYDVDHIVPQSFLKDDSIDNKVLTRSDKNRGKSDNVPSEEVVKKMKNYWRQLLNAKL |
| | ITQRKFDNLTKAERGGLSELDKAGFIKRQLVETRQITKHVAQILDSRMNTKYDENDKLIREVKVITLKSKLVSDFRKDFQF |
| | YKVREINNYHHAHDAYLNAVVGTALIKKYPKLESEFVYGDYKVYDVRKMIAKSEQEIGKATAKYFFYSNIMNFFKTEITLA |
| | NGEIRKRPLIETNGETGEIVWDKGRDFATVRKVLSMPQVNIVKKTEVQTGGFSKESILPKRNSDKLIARKKDWDPKKYGGF |
| | DSPTVAYSVLVVAKVEKGKSKKLKSVKELLGITIMERSSFEKNPIDFLEAKGYKEVKKDLIIKLPKYSLFELENGRKRMLA |
| | SAGELQKGNELALPSKYVNFLYLASHYEKLKGSPEDNEQKQLFVEQHKHYLDEIIEQISEFSKRVILADANLDKVLSAYNK |
| | HRDKPIREQAENI IHLFTLTNLGAPAAFKYFDTTIDRKRYTSTKEVLDATLIHQSITGLYETRIDLSQLGGD |
| | (SEQ ID NO: 37) |
| SaCas9 | MGKRNYILGLDIGITSVGYGIIDYETRDVIDAGVRLFKEANVENNEGRRSKRGARRLKRRRRHRIQRVKKLLFDYNLLTDH |
| Staphylococcus | SELSGINPYEARVKGLSQKLSEEEFSAALLHLAKRRGVHNVNEVEEDTGNELSTKEQISRNSKALEEKYVAELQLERLKKD |
| aureus | GEVRGSINRFKTSDYVKEAKQLLKVQKAYHQLDQSFIDTYIDLLETRRTYYEGPGEGSPFGWKDIKEWYEMLMGHCTYFPE |
| | ELRSVKYAYNADLYNALNDLNNLVITRDENEKLEYYEKFQIIENVFKQKKKPTLKQIAKEILVNEEDIKGYRVTSTGKPEF |
| | TNLKVYHDIKDITARKEIIENAELLDQIAKILTIYQSSEDIQEELTNLNSELTQEEIEQISNLKGYTGTHNLSLKAINLIL |
| | DELWHTNDNQIAIFNRLKLVPKKVDLSQQKEIPTTLVDDFILSPVVKRSFIQSIKVINAIIKKYGLPNDIIIELAREKNSK |
| | DAQKMINEMQKRNRQTNERIEEIIRTTGKENAKYLIEKIKLHDMQEGKCLYSLEAIPLEDLLNNPFNYEVDHIIPRSVSFD |
| | NSFNNKVLVKQEENSKKGNRTPFQYLSSSDSKISYETFKKHILNLAKGKGRISKTKKEYLLEERDINRFSVQKDFINRNLV |
| | DTRYATRGLMNLLRSYFRVNNLDVKVKSINGGFTSFLRRKWKFKKERNKGYKHHAEDALIIANADFIFKEWKKLDKAKKVM |
| | ENQMFEEKQAESMPEIETEQEYKEIFITPHQIKHIKDFKDYKYSHRVDKKPNRKLINDTLYSTRKDDKGNTLIVNNLNGLY |
| | DKDNDKLKKLINKSPEKLLMYHHDPQTYQKLKLIMEQYGDEKNPLYKYYEETGNYLTKYSKKDNGPVIKKIKYYGNKLNAH |
| | LDITDDYPNSRNKVVKLSLKPYRFDVYLDNGVYKFVTVKNLDVIKKENYYEVNSKCYEEAKKLKKISNQAEFIASFYKNDL |
| | IKINGELYRVIGVNNDLLNRIEVNMIDITYREYLENMNDKRPPHIIKTIASKTQSIKKYSTDILGNLYEVKSKKHPQIIKK |
| | (SEQ ID NO: 38) |
| StCas9 | MLFNKCIIISINLDFSNKEKCMTKPYSIGLDIGTNSVGWAVITDNYKVPSKKMKVLGNTSKKYIKKNLLGVLLFDSGITAE |
| Streptococcus | GRRLKRTARRRYTRRRNRILYLQEIFSTEMATLDDAFFQRLDDSFLVPDDKRDSKYPIFGNLVEEKVYHDEFPTIYHLRKY |
| thermophilus | LADSTKKADLRLVYLALAHMIKYRGHFLIEGEFNSKNNDIQKNFQDFLDTYNAIFESDLSLENSKQLEEIVKDKISKLEKK |
| UniProtKB/ | DRILKLFPGEKNSGIFSEFLKLIVGNQADFRKCFNLDEKASLHFSKESYDEDLETLLGYIGDDYSDVFLKAKKLYDAILLS |
| Swiss-Prot: | GFLTVTDNETEAPLSSAMIKRYNEHKEDLALLKEYIRNISLKTYNEVFKDDTKNGYAGYIDGKTNQEDFYVYLKNLLAEFE |
| G3ECR1.2 | GADYFLEKIDREDFLRKQRTFDNGSIPYQIHLQEMRAILDKQAKFYPFLAKNKERIEKILTFRIPYYVGPLARGNSDFAWS |
| Wild type | IRKRNEKITPWNFEDVIDKESSAEAFINRMTSFDLYLPEEKVLPKHSLLYETFNVYNELTKVRFIAESMRDYQFLDSKQKK |
| | DIVRLYFKDKRKVTDKDIIEYLHAIYGYDGIELKGIEKQFNSSLSTYHDLLNIINDKEFLDDSSNEAIIEEIIHTLTIFED |
| | REMIKQRLSKFENIFDKSVLKKLSRRHYTGWGKLSAKLINGIRDEKSGNTILDYLIDDGISNRNFMQLIHDDALSFKKKIQ |
| | KAQIIGDEDKGNIKEVVKSLPGSPAIKKGILQSIKIVDELVKVMGGRKPESIVVEMARENQYTNQGKSNSQQRLKRLEKSL |
| | KELGSKILKENIPAKLSKIDNNALQNDRLYLYYLQNGKDMYTGDDLDIDRLSNYDIDHIIPQAFLKDNSIDNKVLVSSASN |
| | RGKSDDFPSLEVVKKRKTFWYQLLKSKLISQRKFDNLTKAERGGLLPEDKAGFIQRQLVETRQITKHVARLLDEKENNKKD |
| | ENNRAVRTVKIITLKSTLVSQFRKDFELYKVREINDFHHAHDAYLNAVIASALLKKYPKLEPEFVYGDYPKYNSFRERKSA |
| | TEKVYFYSNIMNIFKKSISLADGRVIERPLIEVNEETGESVWNKESDLATVRRVLSYPQVNVVKKVEEQNHGLDRGKPKGL |
| | FNANLSSKPKPNSNENLVGAKEYLDPKKYGGYAGISNSFAVLVKGTIEKGAKKKITNVLEFQGISILDRINYRKDKLNFLL |
| | EKGYKDIELIIELPKYSLFELSDGSRRMLASILSTNNKRGEIHKGNQIFLSQKFVKLLYHAKRISNTINENHRKYVENHKK |
| | EFEELFYYILEFNENYVGAKKNGKLLNSAFQSWQNHSIDELCSSFIGPTGSERKGLFELTSRGSAADFEFLGVKIPRYRDY |
| | TPSSLLKDATLIHQSVTGLYETRIDLAKLGEG (SEQ ID NO: 39) |
| LcCas9 | MKIKNYNLALTPSTSAVGHVEVDDDLNILEPVHHQKAIGVAKFGEGETAEARRLARSARRTTKRRANRINHYFNEIMKPEI |
| Lactobacillus | DKVDPLMFDRIKQAGLSPLDERKEFRTVIFDRPNIASYYHNQFPTIWHLQKYLMITDEKADIRLIYWALHSLLKHRGHFFN |
| crispatus | TTPMSQFKPGKLNLKDDMLALDDYNDLEGLSFAVANSPEIEKVIKDRSMHKKEKIAELKKLIVNDVPDKDLAKRNNKIITQ |
| NCBI | IVNAIMGNSFHLNFIFDMDLDKLTSKAWSFKLDDPELDTKFDAISGSMTDNQIGIFETLQKIYSAISLLDILNGSSNVVDA |
| Reference | KNALYDKHKRDLNLYFKFLNTLPDEIAKTLKAGYTLYIGNRKKDLLAARKLLKVNVAKNFSQDDFYKLINKELKSIDKQGL |
| Sequence: | QTRFSEKVGELVAQNNFLPVQRSSDNVFIPYQLNAITFNKILENQGKYYDFLVKPNPAKKDRKNAPYELSQLMQFTIPYYV |
| WP_133478044.1 | GPLVTPEEQVKSGIPKTSRFAWMVRKDNGAITPWNFYDKVDIEATADKFIKRSIAKDSYLLSELVLPKHSLLYEKYEVENE |
| Wild type | LSNVSLDGKKLSGGVKQILFNEVFKKTNKVNTSRILKALAKHNIPGSKITGLSNPEEFTSSLQTYNAWKKYFPNQIDNFAY |
| | QQDLEKMIEWSTVFEDHKILAKKLDEIEWLDDDQKKFVANTRLRGWGRLSKRLLTGLKDNYGKSIMQRLETTKANFQQIVY |
| | KPEFREQIDKISQAAAKNQSLEDILANSYTSPSNRKAIRKTMSVVDEYIKLNHGKEPDKIFLMFQRSEQEKGKQTEARSKQ |
| | LNRILSQLKADKSANKLFSKQLADEFSNAIKKSKYKLNDKQYFYFQQLGRDALTGEVIDYDELYKYTVLHIIPRSKLTDDS |
| | QNNKVLTKYKIVDGSVALKFGNSYSDALGMPIKAFWTELNRLKLIPKGKLLNLTTDFSTLNKYQRDGYIARQLVETQQIVK |
| | LLATIMQSRFKHTKIIEVRNSQVANIRYQFDYFRIKNLNEYYRGFDAYLAAVVGTYLYKVYPKARRLFVYGQYLKPKKTNQ |
| | ENQDMHLDSEKKSQGFNFLWNLLYGKQDQIFVNGTDVIAFNRKDLITKMNTVYNYKSQKISLAIDYHNGAMFKATLFPRND |
| | RDTAKTRKLIPKKKDYDTDIYGGYTSNVDGYMLLAEIIKRDGNKQYGFYGVPSRLVSELDTLKKTRYTEYEEKLKEIIKPE |
| | LGVDLKKIKKIKILKNKVPFNQVIIDKGSKFFITSTSYRWNYRQLILSAESQQTLMDLVVDPDFSNHKARKDARKNADERL |
| | IKVYEEILYQVKNYMPMFVELHRCYEKLVDAQKTFKSLKISDKAMVLNQILILLHSNATSPVLEKLGYHTRFTLGKKHNLI |
| | SENAVLVTQSITGLKENHVSIKQML (SEQ ID NO: 40) |
| PdCas9 | MTNEKYSIGLDIGTSSIGFAVVNDNNRVIRVKGKNAIGVRLFDEGKAAADRRSFRTTRRSFRTTRRRLSRRRWRLKLLREI |
| Pedicoccus | FDAYITPVDEAFFIRLKESNLSPKDSKKQYSGDILFNDRSDKDFYEKYPTIYHLRNALMTEHRKFDVREIYLAIHHIMKFR |
| damnosus | GHFLNATPANNFKVGRLNLEEKFEELNDIYQRVFPDESIEFRTDNLEQIKEVLLDNKRSRADRQRTLVSDIYQSSEDKDIE |
| NCBI | KRNKAVATEILKASLGNKAKLNVITNVEVDKEAAKEWSITFDSESIDDDLAKIEGQMTDDGHEIIEVLRSLYSGITLSAIV |
| Reference | PENHTLSQSMVAKYDLHKDHLKLFKKLINGMTDTKKAKNLRAAYDGYIDGVKGKVLPQEDFYKQVQVNLDDSAEANEIQTY |
| Sequence: | IDQDIFMPKQRTKANGSIPHQLQQQELDQIIENQKAYYPWLAELNPNPDKKRQQLAKYKLDELVTFRVPYYVGPMITAKDQ |
| WP_062913273.1 | KNQSGAEFAWMIRKEPGNITPWNFDQKVDRMATANQFIKRMTTTDTYLLGEDVLPAQSLLYQKFEVLNELNKIRIDHKPIS |
| Wild type | IEQKQQIFNDLFKQFKNVTIKHLQDYLVSQGQYSKRPLIEGLADEKRFNSSLSTYSDLCGIFGAKLVEENDRQEDLEKIIE |
| | WSTIFEDKKIYRAKLNDLTWLTDDQKEKLATKRYQGWGRLSRKLLVGLKNSEHRNIMDILWITNENFMQIQAEPDFAKLVT |
| | DANKGMLEKTDSQDVINDLYTSPQNKKAIRQILLVVHDIQNAMHGQAPAKIHVEFARGEERNPRRSVQRQRQVEAAYEKVS |
| | NELVSAKVRQEFKEAINNKRDFKDRLFLYFMQGGIDIYTGKQLNIDQLSSYQIDHILPQAFVKDDSLTNRVLTNENQVKAD |
| | SVPIDIFGKKMLSVWGRMKDQGLISKGKYRNLTMNPENISAHTENGFINRQLVETRQVIKLAVNILADEYGDSTQIISVKA |
| | DLSHQMREDFELLKNRDVNDYHHAFDAYLAAFIGNYLLKRYPKLESYFVYGDFKKFTQKETKMRRFNFIYDLKHCDQVVNK |
| | ETGEILWTKDEDIKYIRHLFAYKKILVSHEVREKRGALYNQTIYKAKDDKGSGQESKKLIRIKDDKETKIYGGYSGKSLAY |
| | MTIVQITKKNKVSYRVIGIPTLALARLNKLENDSTENNGELYKIIKPQFTHYKVDKKNGEIIETTDDFKIVVSKVRFQQLI |
| | DDAGQFFMLASDTYKNNAQQLVISNNALKAINNTNITDCPRDDLERLDNLRLDSAFDEIVKKMDKYFSAYDANNFREKIRN |
| | SNLIFYQLPVEDQWENNKITELGKRTVLTRILQGLHANATTTDMSIFKIKTPFGQLRQRSGISLSENAQLIYQSPTGLFER |
| | RVQLNKIK (SEQ ID NO: 41) |
| FnCas9 | MKKQKFSDYYLGFDIGTNSVGWCVTDLDYNVLRFNKKDMWGSRLFEEAKTAAERRVQRNSRRRLKRRKWRLNLLEEIFSNE |
| Fusobaterium | ILKIDSNFFRRLKESSLWLEDKSSKEKFTLENDDNYKDYDFYKQYPTIFHLRNELIKNPEKKDIRLVYLAIHSIFKSRGHF |
| nucleatum | LFEGQNLKEIKNFETLYNNLIAFLEDNGINKIIDKNNIEKLEKIVCDSKKGLKDKEKEFKEIFNSDKQLVAIFKLSVGSSV |
| NCBI | SLNDLFDTDEYKKGEVEKEKISFREQIYEDDKPIYYSILGEKIELLDIAKTFYDFMVLNNILADSQYISEAKVKLYEEHKK |
| Reference | DLKNLKYIIRKYNKGNYDKLFKDKNENNYSAYIGLNKEKSKKEVIEKSRLKIDDLIKNIKGYLPKVEEIEEKDKAIFNKIL |
| Sequence: | NKIELKTILPKQRISDNGTLPYQIHEAELEKILENQSKYYDFLNYEENGIITKDKLLMTFKFRIPYYVGPLNSYHKDKGGN |
| WP_060798984.1 | SWIVRKEEGKILPWNFEQKVDIEKSAEEFIKRMTNKCTYLNGEDVIPKDTFLYSEYVILNELNKVQVNDEFLNEENKRKII |
| | DELFKENKKVSEKKFKEYLLVKQIVDGTIELKGVKDSFNSNYISYIRFKDIFGEKLNLDIYKEISEKSILWKCLYGDDKKI |
| | FEKKIKNEYGDILTKDEIKKINTFKFNNWGRLSEKLLTGIEFINLETGECYSSVMDALRRTNYNLMELLSSKFTLQESINN |
| | ENKEMNEASYRDLIEESYVSPSLKRAIFQTLKIYEEIRKITGRVPKKVFIEMARGGDESMKNKKIPARQEQLKKLYDSCGN |
| | DIANFSIDIKEMKNSLISYDNNSLRQKKLYLYYLQFGKCMYTGREIDLDRLLQNNDTYDIDHIYPRSKVIKDDSFDNLVLV |
| | LKNENAEKSNEYPVKKEIQEKMKSFWRFLKEKNFISDEKYKRLTGKDDFELRGFMARQLVNVRQTTKEVGKILQQIEPEIK |
| | IVYSKAEIASSFREMFDFIKVRELNDTHHAKDAYLNIVAGNVYNTKFTEKPYRYLQEIKENYDVKKIYNYDIKNAWDKENS |
| | LEIVKKNMEKNTVNITRFIKEKKGQLFDLNPIKKGETSNEIISIKPKVYNGKDDKLNEKYGYYKSLNPAYFLYVEHKEKNK |
| | RIKSFERVNLVDVNNIKDEKSLVKYLIENKKLVEPRVIKKVYKRQVILINDYPYSIVTLDSNKLMDFENLKPLFLENKYEK |
| | ILKNVIKFLEDNQGKSEENYKFIYLKKKDRYEKNETLESVKDRYNLEFNEMYDKFLEKLDSKDYKNYMNNKKYQELLDVKE |
| | KFIKLNLFDKAFTLKSFLDLFNRKTMADFSKVGLTKYLGKIQKISSNVLSKNELYLLEESVTGLFVKKIKI (SEQ ID |
| | NO: 42) |
| EcCas9 | MNKYYLGLDMGSASVGWAVTDENYHLVRRKGKDLWGVRTFDVAQTAKERRITRGNRRRQDRRKQRIQILQELLGEEVLKTD |
| Enterococcus | PGFFHRMKESRYVVEDKRTLDGKQVELPYALFVDKDYTDKEYYKQFPTINHLIVYLMTTSDTPDIRLVYLALHYYMKNRGN |
| cecorum | FLHSGDINNVKDINDILEQLDNVLETFLDGWNLKLKSYVEDIKNIYNRDLGRGERKKAFVNTLGAKTKAEKAFCSLISGGS |
| NCBI | TNLAELFDDSSLKEIETPKIEFASSSLEDKIDGIQEALEDRFAVIEAAKRLYDWKTLTDILGDSSSLAEARVNSYQMHHEQ |
| Reference | LLELKSLVKEYLDRKVFQEVFVSLNVANNYPAYIGHTKINGKKKELEVKRTKRNDFYSYVKKQVIEPIKKKVSDEAVLTKL |
| Sequence: | SEIESLIEVDKYLPLQVNSDNGVIPYQVKLNELTRIFDNLENRIPVLRENRDKIIKTFKFRIPYYVGSLNGVVKNGKCTNW |
| WP_047338501.1 | MVRKEEGKIYPWNFEDKVDLEASAEQFIRRMTNKCTYLVNEDVLPKYSLLYSKYLVLSELNNLRIDGRPLDVKIKQDIYEN |
| Wild type | VFKKNRKVTLKKIKKYLLKEGIITDDDALSGLADDVKSSLTAYRDFKEKLGHLDLSEAQMENIILNITLFGDDKKLLKKRL |
| | AALYPFIDDKSLNRIATLNYRDWGRLSERFLSGITSVDQETGELRTIIQCMYETQANLMQLLAEPYHFVEAIEKENPKVDL |
| | ESISYRIVNDLYVSPAVKRQIWQTLLVIKDIKQVMKHDPERIFIEMAREKQESKKTKSRKQVLSEVYKKAKEYEHLFEKLN |
| | SLTEEQLRSKKIYLYFTQLGKCMYSGEPIDFENLVSANSNYDIDHIYPQSKTIDDSFNNIVLVKKSLNAYKSNHYPIDKNI |
| | RDNEKVKTLWNTLVSKGLITKEKYERLIRSTPFSDEELAGFIARQLVETRQSTKAVAEILSNWFPESEIVYSKAKNVSNER |
| | QDFEILKVRELNDCHHAHDAYLNIVVGNAYHTKFTNSPYRFIKNKANQEYNLRKLLQKVNKIESNGVVAWVGQSENNPGTI |
| | ATVKKVIRRNTVLISRMVKEVDGQLFDLTLMKKGKGQVPIKSSDERLTDISKYGGYNKATGAYFTFVKSKKRGKVVRSFEY |
| | VPLHLSKQFENNNELLKEYIEKDRGLTDVEILIPKVLINSLFRYNGSLVRITGRGDTRLLLVHEQPLYVSNSFVQQLKSVS |
| | SYKLKKSENDNAKLTKTATEKLSNIDELYDGLLRKLDLPIYSYWFSSIKEYLVESRTKYIKLSIEEKALVIFEILHLFQSD |
| | AQVPNLKILGLSTKPSRIRIQKNLKDTDKMSIIHQSPSGIFEHEIELTSL (SEQ ID NO: 444) |
| AhCas9 | MQNGFLGITVSSEQVGWAVTNPKYELERASRKDLWGVRLFDKAETAEDRRMFRTNRRLNQRKKNRIHYLRDIFHEEVNQKD |
| Anaerostipes | PNFFQQLDESNFCEDDRIVEFNFDTNLYKNQFPTVYHLRKYLMETKDKPDIRLVYLAFSKFMKNRGHFLYKGNLGEVMDFE |
| hadrus | NSMKGFCESLEKFNIDFPTLSDEQVKEVRDILCDHKIAKTVKKKNIITITKVKSKTAKAWIGLFCGCSVPVKVLFQDIDEE |
| NCBI | IVTDPEKISFEDASYDDYIANIEKGVGIYYEAIVSAKMLFDWSILNEILGDHQLLSDAMIAEYNKHHDDLKRLQKIIKGTG |
| Reference | SRELYQDIFINDVSGNYVCYVGHAKTMSSADQKQFYTFLKNRLKNVNGISSEDAEWIDTEIKNGTLLPKQTKRDNSVIPHQ |
| Sequence: | LQLREFELILDNMQEMYPFLKENREKLLKIFNFVIPYYVGPLKGVVRKGESTNWMVPKKDGVIHPWNFDEMVDKEASAECF |
| WP_044924278.1 | ISRMTGNCSYLFNEKVLPKNSLLYETFEVLNELNPLKINGEPISVELKQRIYEQLFLTGKKVTKKSLTKYLIKNGYDKDIE |
| Wild type | LSGIDNEFHSNLKSHIDFEDYDNLSDEEVEQIILRITVFEDKQLLKDYLNREFVKLSEDERKQICSLSYKGWGNLSEMLLN |
| | GITVTDSNGVEVSVMDMLWNTNLNLMQILSKKYGYKAEIEHYNKEHEKTIYNREDLMDYLNIPPAQRRKVNQLITIVKSLK |
| | KTYGVPNKIFFKISREHQDDPKRTSSRKEQLKYLYKSLKSEDEKHLMKELDELNDHELSNDKVYLYFLQKGRCIYSGKKLN |
| | LSRLRKSNYQNDIDYIYPLSAVNDRSMNNKVLTGIQENRADKYTYFPVDSEIQKKMKGFWMELVLQGFMTKEKYFRLSREN |
| | DFSKSELVSFIEREISDNQQSGRMIASVLQYYFPESKIVFVKEKLISSFKRDFHLISSYGHNHLQAAKDAYITIVVGNVYH |
| | TKFTMDPAIYFKNHKRKDYDLNRLFLENISRDGQIAWESGPYGSIQTVRKEYAQNHIAVTKRVVEVKGGLFKQMPLKKGHG |
| | EYPLKTNDPRFGNIAQYGGYTNVTGSYFVLVESMEKGKKRISLEYVPVYLHERLEDDPGHKLLKEYLVDHRKLNHPKILLA |
| | KVRKNSLLKIDGFYYRLNGRSGNALILTNAVELIMDDWQTKTANKISGYMKRRAIDKKARVYQNEFHIQELEQLYDFYLDK |
| | LKNGVYKNRKNNQAELIHNEKEQFMELKTEDQCVLLTEIKKLFVCSPMQADLTLIGGSKHTGMIAMSSNVTKADFAVIAED |
| | PLGLRNKVIYSHKGEK (SEQ ID NO: 44) |
| KvCas9 | MSQNNNKIYNIGLDIGDASVGWAVVDEHYNLLKRHGKHMWGSRLFTQANTAVERRSSRSTRRRYNKRRERIRLLREIMEDM |
| Kandleria | VLDVDPTFFIRLANVSFLDQEDKKDYLKENYHSNYNLFIDKDFNDKTYYDKYPTIYHLRKHLCESKEKEDPRLIYLALHHI |
| vitulina | VKYRGNFLYEGQKFSMDVSNIEDKMIDVLRQFNEINLFEYVEDRKKIDEVLNVLKEPLSKKHKAEKAFALFDTTKDNKAAY |
| NCBI | KELCAALAGNKFNVTKMLKEAELHDEDEKDISFKFSDATFDDAFVEKQPLLGDCVEFIDLLHDIYSWVELQNILGSAHTSE |
| Reference | PSISAAMIQRYEDHKNDLKLLKDVIRKYLPKKYFEVFRDEKSKKNNYCNYINHPSKTPVDEFYKYIKKLIEKIDDPDVKTI |
| Sequence: | LNKIELESFMLKQNSRINGAVPYQMQLDELNKILENQSVYYSDLKDNEDKIRSILTFRIPYYFGPLNITKDRQFDWIIKKE |
| WP_031589969.1 | GKENERILPWNANEIVDVDKTADEFIKRMRNFCTYFPDEPVMAKNSLTVSKYEVLNEINKLRINDHLIKRDMKDKMLHTLF |
| Wild type | MDHKSISANAMKKWLVKNQYFSNTDDIKIEGFQKENACSTSLTPWIDFTKIFGKINESNYDFIEKIIYDVTVFEDKKILRR |
| | RLKKEYDLDEEKIKKILKLKYSGWSRLSKKLLSGIKTKYKDSTRTPETVLEVMERTNMNLMQVINDEKLGFKKTIDDANST |
| | SVSGKFSYAEVQELAGSPAIKRGIWQALLIVDEIKKIMKHEPAHVYIEFARNEDEKERKDSFVNQMLKLYKDYDFEDETEK |
| | EANKHLKGEDAKSKIRSERLKLYYTQMGKCMYTGKSLDIDRLDTYQVDHIVPQSLLKDDSIDNKVLVLSSENQRKLDDLVI |
| | PSSIRNKMYGFWEKLFNNKIISPKKFYSLIKTEFNEKDQERFINRQIVETRQITKHVAQIIDNHYENTKVVTVRADLSHQF |
| | RERYHIYKNRDINDFHHAHDAYIATILGTYIGHRFESLDAKYIYGEYKRIFRNQKNKGKEMKKNNDGFILNSMRNIYADKD |
| | TGEIVWDPNYIDRIKKCFYYKDCFVTKKLEENNGTFFNVTVLPNDTNSDKDNTLATVPVNKYRSNVNKYGGFSGVNSFIVA |
| | IKGKKKKGKKVIEVNKLTGIPLMYKNADEEIKINYLKQAEDLEEVQIGKEILKNQLIEKDGGLYYIVAPTEIINAKQLILN |
| | ESQTKLVCEIYKAMKYKNYDNLDSEKIIDLYRLLINKMELYYPEYRKQLVKKFEDRYEQLKVISIEEKCNIIKQILATLHC |
| | NSSIGKIMYSDFKISTTIGRINGRTISLDDISFIAESPTGMYSKKYKL (SEQ ID NO: 45) |
| EfCas9 | MRLFEEGHTAEDRRLKRTARRRISRRRNRLRYLQAFFEEAMTDLDENFFARLQESFLVPEDKKWHRHP IFAKLEDEVAYHE |
| Enterococcus | TYPTIYHLRKKLADSSEQADLRLIYLALAHIVKYRGHFLIEGKLSTENTSVKDQFQQFMVIYNQTFVNGESRLVSAPLPES |
| faecalis | VLIEEELTEKASRTKKSEKVLQQFPQEKANGLFGQFLKLMVGNKADFKKVFGLEEEAKITYASESYEEDLEGILAKVGDEY |
| NCBI | SDVFLAAKNVYDAVELSTILADSDKKSHAKLSSSMIVRFTEHQEDLKKFKRFIRENCPDEYDNLFKNEQKDGYAGYIAHAG |
| Reference | KVSQLKFYQYVKKIIQDIAGAEYFLEKIAQENFLRKQRTFDNGVIPHQIHLAELQAIIHRQAAYYPFLKENQEKIEQLVTF |
| Sequence: | RIPYYVGPLSKGDASTFAWLKRQSEEPIRPWNLQETVDLDQSATAFIERMTNFDTYLPSEKVLPKHSLLYEKFMVFNELTK |
| WP_016631044.1 | ISYTDDRGIKANFSGKEKEKIFDYLFKTRRKVKKKDIIQFYRNEYNTEIVTLSGLEEDQFNASFSTYQDLLKCGLTRAELD |
| Wild type | HPDNAEKLEDIIKILTIFEDRQRIRTQLSTFKGQFSAEVLKKLERKHYTGWGRLSKKLINGIYDKESGKTILDYLVKDDGV |
| | SKHYNRNFMQLINDSQLSFKNAIQKAQSSEHEETLSETVNELAGSPAIKKGIYQSLKIVDELVAIMGYAPKRIVVEMAREN |
| | QTTSTGKRRSIQRLKIVEKAMAEIGSNLLKEQPTTNEQLRDTRLFLYYMQNGKDMYTGDELSLHRLSHYDIDHIIPQSFMK |
| | DDSLDNLVLVGSTENRGKSDDVPSKEVVKDMKAYWEKLYAAGLISQRKFQRLTKGEQGGLTLEDKAHFIQRQLVETRQITK |
| | NVAGILDQRYNAKSKEKKVQIITLKASLTSQFRSIFGLYKVREVNDYHHGQDAYLNCVVATTLLKVYPNLAPEFVYGEYPK |
| | FQTFKENKATAKAIIYTNLLRFFTEDEPRFTKDGEILWSNSYLKTIKKELNYHQMNIVKKVEVQKGGFSKESIKPKGPSNK |
| | LIPVKNGLDPQKYGGFDSPVVAYTVLFTHEKGKKPLIKQEILGITIMEKTRFEQNPILFLEEKGFLRPRVLMKLPKYTLYE |
| | FPEGRRRLLASAKEAQKGNQMVLPEHLLTLLYHAKQCLLPNQSESLAYVEQHQPEFQEILERVVDFAEVHTLAKSKVQQIV |
| | KLFEANQTADVKEIAASFIQLMQFNAMGAPSTFKFFQKDIERARYTSIKEIFDATIIYQSPTGLYETRRKVVD |
| | (SEQ ID NO: 46) |
| Staphylococcus | KRNYILGLDIGITSVGYGIIDYETRDVIDAGVRLFKEANVENNEGRRSKRGARRLKRRRRHRIQRVKKLLFDYNLLTDHSE |
| aureus | LSGINPYEARVKGLSQKLSEEEFSAALLHLAKRRGVHNVNEVEEDTGNELSTKEQISRNSKALEEKYVAELQLERLKKDGE |
| Cas9 | VRGSINRFKTSDYVKEAKQLLKVQKAYHQLDQSFIDTYIDLLETRRTYYEGPGEGSPFGWKDIKEWYEMLMGHCTYFPEEL |
| | RSVKYAYNADLYNALNDLNNLVITRDENEKLEYYEKFQIIENVFKQKKKPTLKQIAKEILVNEEDIKGYRVTSTGKPEFTN |
| | LKVYHDIKDITARKEIIENAELLDQIAKILTIYQSSEDIQEELTNLNSELTQEEIEQISNLKGYTGTHNLSLKAINLILDE |
| | LWHTNDNQIAIFNRLKLVPKKVDLSQQKEIPTTLVDDFILSPVVKRSFIQSIKVINAIIKKYGLPNDIIIELAREKNSKDA |
| | QKMINEMQKRNRQTNERIEEIIRTTGKENAKYLIEKIKLHDMQEGKCLYSLEAIPLEDLLNNPFNYEVDHIIPRSVSEDNS |
| | FNNKVLVKQEENSKKGNRTPFQYLSSSDSKISYETFKKHILNLAKGKGRISKTKKEYLLEERDINRFSVQKDFINRNLVDT |
| | RYATRGLMNLLRSYFRVNNLDVKVKSINGGFTSFLRRKWKFKKERNKGYKHHAEDALIIANADFIFKEWKKLDKAKKVMEN |
| | QMFEEKQAESMPEIETEQEYKEIFITPHQIKHIKDFKDYKYSHRVDKKPNRELINDTLYSTRKDDKGNTLIVNNLNGLYDK |
| | DNDKLKKLINKSPEKLLMYHHDPQTYQKLKLIMEQYGDEKNPLYKYYEETGNYLTKYSKKDNGPVIKKIKYYGNKLNAHLD |
| | ITDDYPNSRNKVVKLSLKPYRFDVYLDNGVYKFVTVKNLDVIKKENYYEVNSKCYEEAKKLKKISNQAEFIASFYNNDLIK |
| | INGELYRVIGVNNDLLNRIEVNMIDITYREYLENMNDKRPPRIIKTIASKTQSIKKYSTDILGNLYEVKSKKHPQIIKKG |
| | (SEQ ID NO: 47) |
| Geobacillus | MKYKIGLDIGITSIGWAVINLDIPRIEDLGVRIFDRAENPKTGESLALPRRLARSARRRLRRRKHRLERIRRLFVREGILT |
| thermodenitri- | KEELNKLFEKKHEIDVWQLRVEALDRKLNNDELARILLHLAKRRGFRSNRKSERTNKENSTMLKHIEENQSILSSYRTVAE |
| ficans | MVVKDPKFSLHKRNKEDNYTNTVARDDLEREIKLIFAKQREYGNIVCTEAFEHEYISIWASQRPFASKDDIEKKVGFCTFE |
| Cas9 | PKEKRAPKATYTFQSFTVWEHINKLRLVSPGGIRALTDDERRLIYKQAFHKNKITFHDVRTLLNLPDDTRFKGLLYDRNTT |
| | LKENEKVRFLELGAYHKIRKAIDSVYGKGAAKSFRPIDFDTFGYALTMFKDDTDIRSYLRNEYEQNGKRMENLADKVYDEE |
| | LIEELLNLSFSKFGHLSLKALRNILPYMEQGEVYSTACERAGYTFTGPKKKQKTVLLPNIPPIANPVVMRALTQARKVVNA |
| | IIKKYGSPVSIHIELARELSQSFDERRKMQKEQEGNRKKNETAIRQLVEYGLTLNPTGLDIVKFKLWSEQNGKCAYSLQPI |
| | EIERLLEPGYTEVDHVIPYSRSLDDSYTNKVLVLTKENREKGNRTPAEYLGLGSERWQQFETFVLINKQFSKKKRDRLLRL |
| | HYDENEENEFKNRNLNDTRYISRFLANFIREHLKFADSDDKQKVYTVNGRITAHLRSRWNENKNREESNLHHAVDAAIVAC |
| | TTPSDIARVTAFYQRREQNKELSKKTDPQFPQPWPHFADELQARLSKNPKESIKALNLGNYDNEKLESLQPVFVSRMPKRS |
| | ITGAAHQETLRRYIGIDERSGKIQTVVKKKLSEIQLDKTGHFPMYGKESDPRTYEAIRQRLLEHNNDPKKAFQEPLYKPKK |
| | NGELGPIIRTIKIIDTTNQVIPLNDGKTVAYNSNIVRVDVFEKDGKYYCVPIYTIDMMKGILPNKAIEPNKPYSEWKEMTE |
| | DYTFRFSLYPNDLIRIEFPREKTIKTAVGEEIKIKDLFAYYQTIDSSNGGLSLVSHDNNFSLRSIGSRTLKRFEKYQVDVL |
| | GNIYKVRGEKRVGVASSSHSKAGETIRPL (SEQ ID NO: 48) |
| ScCas9 | MEKKYSIGLDIGTNSVGWAVITDDYKVPSKKFKVLGNTNRKSIKKNLMGALLFDSGETAEATRLKRTARRRYTRRKNRIRY |
| S. canis | LQEIFANEMAKLDDSFFQRLEESFLVEEDKKNERHPIFGNLADEVAYHRNYPTIYHLRKKLADSPEKADLRLIYLALAHII |
| 1375 AA | KFRGHFLIEGKLNAENSDVAKLFYQLIQTYNQLFEESPLDEIEVDAKGILSARLSKSKRLEKLIAVFPNEKKNGLFGNIIA |
| 159.2 kDa | LALGLTPNFKSNFDLTEDAKLQLSKDTYDDDLDELLGQIGDQYADLFSAAKNLSDAILLSDILRSNSEVTKAPLSASMVKR |
| | YDEHHQDLALLKTLVRQQFPEKYAEIFKDDTKNGYAGYVGIGIKHRKRTTKLATQEEFYKFIKPILEKMDGAEELLAKLNR |
| | DDLLRKQRTFDNGSIPHQIHLKELHAILRRQEEFYPFLKENREKIEKILTFRIPYYVGPLARGNSRFAWLTRKSEEAITPW |
| | NFEEVVDKGASAQSFIERMTNFDEQLPNKKVLPKHSLLYEYFTVYNELTKVKYVTERMRKPEFLSGEQKKAIVDLLFKTNR |
| | KVTVKQLKEDYFKKIECFDSVEIIGVEDRFNASLGTYHDLLKIIKDKDFLDNEENEDILEDIVLTLTLFEDREMIEERLKT |
| | YAHLFDDKVMKQLKRRHYTGWGRLSRKMINGIRDKQSGKTILDFLKSDGFSNRNFMQLIHDDSLTFKEEIEKAQVSGQGDS |
| | LHEQIADLAGSPAIKKGILQTVKIVDELVKVMGHKPENIVIEMARENQTTTKGLQQSRERKKRIEEGIKELESQILKENPV |
| | ENTQLQNEKLYLYYLQNGRDMYVDQELDINRLSDYDVDHIVPQSFIKDDSIDNKVLTRSVENRGKSDNVPSEEVVKKMKNY |
| | WRQLLNAKLITQRKFDNLTKAERGGLSEADKAGFIKRQLVETRQITKHVARILDSRMNTKRDKNDKPIREVKVITLKSKLV |
| | SDFRKDFQLYKVRDINNYHHAHDAYLNAVVGTALIKKYPKLESEFVYGDYKVYDVRKMIAKSEQEIGKATAKRFFYSNIMN |
| | FFKTEVKLANGEIRKRPLIETNGETGEVVWNKEKDFATVRKVLAMPQVNIVKKTEVQTGGFSKESILSKRESAKLIPRKKG |
| | WDTRKYGGFGSPTVAYSILVVAKVEKGKAKKLKSVKVLVGITIMEKGSYEKDPIGFLEAKGYKDIKKELIFKLPKYSLFEL |
| | ENGRRRMLASATELQKANELVLPQHLVRLLYYTQNISATTGSNNLGYIEQHREEFKEIFEKIIDFSEKYILKNKVNSNLKS |
| | SFDEQFAVSDSILLSNSFVSLLKYTSFGASGGFTFLDLDVKQGRLRYQTVTEVLDATLIYQSITGLYETRTDLSQLGGD |
| | (SEQ ID NO: 49) |

The base editors described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or naturally occurring enzymes, such as, Cas9. Cas9 homologs and/or orthologs have been described in various species, including, but not limited to, S. pyogenes and S. thermophilus. Preferably, the Cas moiety is configured (e.g, mutagenized, recombinantly engineered, or otherwise obtained from nature) as a nickase, i.e., capable of cleaving only a single strand of the target doubpdditional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase. In some embodiments, the Cas9 protein comprises an amino acid sequence that is at least 80% identical to the amino acid sequence of a Cas9 protein as provided by any one of the variants of Table 3. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

### (3) Dead Cas9 Variant

In certain embodiments, the base editors described herein may include a dead Cas9, e.g., dead SpCas9, which has no nuclease activity due to one or more mutations that inactive both nuclease domains of Cas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivate of the endogneous Cas9 nuclease activity (e.g., nCas9 or dCas9, respectively). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of Cas9 (e.g., variants of Cas9 from Streptococcus pyogenes (NCBI Reference Sequence: NC_017053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In one embodiment, the dead Cas9 may be based on the canonical SpCas9 sequence of Q99ZW2 and may have the following sequence, which comprises a D10A and an H8 10A substitutions (underlined and bolded), or a variant be variant of SEQ ID NO: 27 having at least 80%, at least 85%, at least 90%, at least 95%, or at least 99% sequence identity thereto:

| Description | Sequence | SEQ ID NO: |
|---|---|---|
| dead Cas9 or | MDKKYSIGLXIGTNSVGWAVITDEYKVPSKKFKVLGNTDRHSIKKNLIGALLFDSGETAEA | 50 |
| dCas9 | TRLKRTARRRYTRRKNRICYLQEIFSNEMAKVDDSFFHRLEESFLVEEDKKHERHPIFGNI | |
| Streptococcus | VDEVAYHEKYPTIYHLRKKLVDSTDKADLRLIYLALAHMIKFRGHFLIEGDLNPDNSDVDK | |
| pyogenes | LFIQLVQTYNQLFEENPINASGVDAKAILSARLSKSRRLENLIAQLPGEKKNGLFGNLIAL | |
| Q99ZW2 Cas9 | SLGLTPNFKSNFDLAEDAKLQLSKDTYDDDLDNLLAQIGDQYADLFLAAKNLSDAILLSDI | |
| with D10X | LRVNTEITKAPLSASMIKRYDEHHQDLILLKALVRQQLPEKYKEIFFDQSKNGYAGYIDGG | |
| and H810X | ASQEEFYKFIKPILEKMDGTEELLVKLNREDLLRKQRTEDNGSIPHQIHLGELHAILRRQE | |
| Where ″X″ is | DFYPFLKDNREKIEKILTFRIPYYVGPLARGNSRFAWMTRKSEETITPWNFEEVVDKGASA | |
| any amino | QSFIERMTNFDKNLPNEKVLPKHSLLYEYFTVYNELTKVKYVTEGMRKPAFLSGEQKKAIV | |
| acid | DLLFKTNRKVTVKQLKEDYFKKIECFDSVEISGVEDRENASLGTYHDLLKIIKDKDELDNE | |
| | ENEDILEDIVLTLILFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDXIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | LDATLIHQSITGLYETRIDLSQLGGD | |
| dead Cas9 or | MDKKYSIGLAIGTNSVGWAVITDEYKVPSKKFKVLGNTDRHSIKKNLIGALLFDSGETAEA | 51 |
| dCas9 | TRLKRTARRRYTRRKNRICYLQEIFSNEMAKVDDSFFHRLEESFLVEEDKKHERHPIFGNI | |
| Streptococcus | VDEVAYHEKYPTIYHLRKKLVDSTDKADLRLIYLALAHMIKERGHFLIEGDLNPDNSDVDK | |
| pyogenes | LFIQLVQTYNQLFEENPINASGVDAKAILSARLSKSRRLENLIAQLPGEKKNGLFGNLIAL | |
| Q99ZW2 Cas9 | SLGLTPNFKSNFDLAEDAKLQLSKDTYDDDLDNLLAQIGDQYADLFLAAKNLSDAILLSDI | |
| with D10A | LRVNTEITKAPLSASMIKRYDEHHQDLTLLKALVRQQLPEKYKEIFFDQSKNGYAGYIDGG | |
| and H810A | ASQEEFYKFIKPILEKMDGTEELLVKLNREDLLRKQRTEDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDELDNE | |
| | ENEDILEDIVLTLILFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDAIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | ILDATLIHQSITGLYETRIDLSQLGGD | |

### (4) Cas9 Nickase Variant

In one embodiment, the base editors described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In various embodiments, the Cas9 nickase can having a mutation in the RuvC nuclease domain and have one of the following amino acid sequences, or a variant thereof having an amino acid sequence that has at least 80%, at least 85%, at least 90%, at least 95%, or at least 99% sequence identity thereto.

| | | SEQ |
|---|---|---|
| | | ID |
| Description | Sequence | NO: |
| Cas9 nickase | MDKKYSIGLXIGTNSVGWAVITDEYKVPSKKFKVLGNTDRHSIKKNLIGALLFDSGETAEA | 52 |
| Streptococcus | TRLKRTARRRYTRRKNRICYLQEIFSNEMAKVDDSFFHRLEESFLVEEDKKHERHPIFGNI | |
| pyogenes | VDEVAYHEKYPTIYHLRKKLVDSTDKADLRLIYLALAHMIKERGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with D10X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTILLKALVRQQLPEKYKEIFFDQSKNGYAGYIDGG | |
| any | ASQEEFYKFIKPILEKMDGTEELLVKLNREDLLRKQRTFDNGSIPHQIHLGELHAILRRQE | |
| alternate | DFYPFLKDNREKIEKILTFRIPYYVGPLARGNSRFAWMTRKSEETITPWNFEEVVDKGASA | |
| amino acid | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDELDNE | |
| | ENEDILEDIVLTLILFEDREMIEERLKTYAHLEDDKVMKQLKRRRYTGWGRLSRKLINGIR | |
| | DKQSGKTILDFLKSDGFANRNEMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKEDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIEINGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYEDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | 53 |
| Streptococcus | TRLKRTARRRYTRRKNRICYLQEIFSNEMAKVDDSFFHRLEESELVEEDKKHERHPIFGNI | |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with E762X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLILLKALVRQQLPEKYKEIFFDQSKNGYAGYIDGG | |
| any | ASQEEFYKFIKPILEKMDGTEELLVKLNREDLLRKQRTFDNGSIPHQIHLGELHAILRRQE | |
| alternate | DFYPFLKDNREKIEKILTFRIPYYVGPLARGNSRFAWMTRKSEETITPWNFEEVVDKGASA | |
| amino acid | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRENASLGTYHDLLKIIKDKDELDNE | |
| | ENEDILEDIVLTLTLFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIXMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKEDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | LDATLIHQSITGLYETRIDLSQLGGD | |
| Cas9 | MDKKYSIGLDIGTNSVGWAVITDEYKVPSKKFKVLGNTDRHSIKKNLIGALLEDSGETAEA | 54 |
| nickase | TRLKRTARRRYTRRKNRICYLQEIFSNEMAKVDDSFFHRLEESELVEEDKKHERHPIFGNI | |
| Streptococcus | VDEVAYHEKYPTIYHLRKKLVDSTDKADLRLIYLALAHMIKFRGHFLIEGDLNPDNSDVDK | |
| pyogenes | LFIQLVQTYNQLFEENPINASGVDAKAILSARLSKSRRLENLIAQLPGEKKNGLEGNLIAL | |
| Q99ZW2 Cas9 | SLGLTPNFKSNFDLAEDAKLQLSKDTYDDDLDNLLAQIGDQYADLFLAAKNLSDAILLSDI | |
| with H983X, | LRVNTEITKAPLSASMIKRYDEHHQDLTLLKALVRQQLPEKYKEIFFDQSKNGYAGYIDGG | |
| wherein X is | ASQEEFYKFIKPILEKMDGTEELLVKLNREDLLRKQRTEDNGSIPHQIHLGELHAILRRQE | |
| any | DFYPFLKDNREKIEKILTFRIPYYVGPLARGNSRFAWMTRKSEETITPWNFEEVVDKGASA | |
| alternate | QSFIERMTNFDKNLPNEKVLPKHSLLYEYFTVYNELTKVKYVTEGMRKPAFLSGEQKKAIV | |
| amino acid | DLLFKTNRKVTVKQLKEDYFKKIECFDSVEISGVEDRFNASLGTYHDLLKIIKDKDELDNE | |
| | ENEDILEDIVLTLILFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHXAHDAYLNAVVGTALIKKYPKLESEFVYGDYKVYDVRKMIAKSEQEIGKATAKYF | |
| | FYSNIMNFFKTEITLANGEIRKRPLIEINGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 | MDKKYSIGLDIGTNSVGWAVITDEYKVPSKKFKVLGNTDRHSIKKNLIGALLFDSGETAEA | 55 |
| nickase | TRLKRTARRRYTRRKNRICYLQEIFSNEMAKVDDSFFHRLEESFLVEEDKKHERHPIFGNI | |
| Streptococcus | VDEVAYHEKYPTIYHLRKKLVDSTDKADLRLIYLALAHMIKFRGHFLIEGDLNPDNSDVDK | |
| pyogenes | LFIQLVQTYNQLFEENPINASGVDAKAILSARLSKSRRLENLIAQLPGEKKNGLFGNLIAL | |
| Q99ZW2 Cas9 | SLGLTPNFKSNFDLAEDAKLQLSKDTYDDDLDNLLAQIGDQYADLFLAAKNLSDAILLSDI | |
| with D986X, | LRVNTEITKAPLSASMIKRYDEHHQDLILLKALVRQQLPEKYKEIFFDQSKNGYAGYIDGG | |
| wherein X is | ASQEEFYKFIKPILEKMDGTEELLVKLNREDLLRKQRTFDNGSIPHQIHLGELHAILRRQE | |
| any | DFYPFLKDNREKIEKILTFRIPYYVGPLARGNSRFAWMTRKSEETITPWNFEEVVDKGASA | |
| alternate | QSFIERMTNFDKNLPNEKVLPKHSLLYEYFTVYNELTKVKYVTEGMRKPAFLSGEQKKAIV | |
| amino acid | DLLFKTNRKVTVKQLKEDYFKKIECFDSVEISGVEDRFNASLGTYHDLLKIIKDKDELDNE | |
| | ENEDILEDIVLTLILFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHXAYLNAVVGTALIKKYPKLESEFVYGDYKVYDVRKMIAKSEQEIGKATAKYF | |
| | FYSNIMNFFKTEITLANGEIRKRPLIEINGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | LDATLIHQSITGLYETRIDLSQLGGD | |
| Cas9 nickase | MDKKYSIGLAIGTNSVGWAVITDEYKVPSKKFKVLGNTDRHSIKKNLIGALLFDSGETAEA | 56 |
| Streptococcus | TRLKRTARRRYTRRKNRICYLQEIFSNEMAKVDDSFFHRLEESFLVEEDKKHERHPIFGNI | |
| pyogenes | VDEVAYHEKYPTIYHLRKKLVDSTDKADLRLIYLALAHMIKERGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with D10A | SLGLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYIDGG | |
| | ASQEEFYKFIKPILEKMDGTEELLVKLNREDLLRKQRTEDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDELDNE | |
| | ENEDILEDIVLILTLFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIEINGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | LDATLIHQSITGLYETRIDLSQLGGD | |
| Cas9 | MDKKYSIGLDIGTNSVGWAVITDEYKVPSKKFKVLGNTDRHSIKKNLIGALLEDSGETAEA | 57 |
| nickase | TRLKRTARRRYTRRKNRICYLQEIFSNEMAKVDDSFFHRLEESELVEEDKKHERHPIFGNI | |
| Streptococcus | VDEVAYHEKYPTIYHLRKKLVDSTDKADLRLIYLALAHMIKFRGHFLIEGDLNPDNSDVDK | |
| pyogenes | LFIQLVQTYNQLFEENPINASGVDAKAILSARLSKSRRLENLIAQLPGEKKNGLEGNLIAL | |
| Q99ZW2 Cas9 | SLGLTPNFKSNFDLAEDAKLQLSKDTYDDDLDNLLAQIGDQYADLFLAAKNLSDAILLSDI | |
| with E762A | LRVNTEITKAPLSASMIKRYDEHHQDLTLLKALVRQQLPEKYKEIFFDQSKNGYAGYIDGG | |
| | ASQEEFYKFIKPILEKMDGTEELLVKLNREDLLRKQRTFDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRENASLGTYHDLLKIIKDKDFLDNE | |
| | ENEDILEDIVLTLTLFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIAMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYEDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | 58 |
| Streptococcus | TRLKRTARRRYTRRKNRICYLQEIFSNEMAKVDDSFFHRLEESFLVEEDKKHERHPIFGNI | |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with H983A | SLGLTPNFKSNFDLAEDAKLQLSKDTYDDDLDNLLAQIGDQYADLFLAAKNLSDAILLSDI | |
| | LRVNTEITKAPLSASMIKRYDEHHQDLILLKALVRQQLPEKYKEIFFDQSKNGYAGYIDGG | |
| | ASQEEFYKFIKPILEKMDGTEELLVKLNREDLLRKQRTEDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDELDNE | |
| | ENEDILEDIVLILILFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHAAHDAYLNAVVGTALIKKYPKLESEFVYGDYKVYDVRKMIAKSEQEIGKATAKYF | |
| | FYSNIMNFFKTEITLANGEIRKRPLIEINGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | 59 |
| Streptococcus | TRLKRTARRRYTRRKNRICYLQEIFSNEMAKVDDSFFHRLEESFLVEEDKKHERHPIFGNI | |
| pyogenes | VDEVAYHEKYPTIYHLRKKLVDSTDKADLRLIYLALAHMIKERGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with D986A | SLGLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYIDGG | |
| | ASQEEFYKFIKPILEKMDGTEELLVKLNREDLLRKQRTFDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDELDNE | |
| | ENEDILEDIVLTLILFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHAAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | LDATLIHQSITGLYETRIDLSQLGGD | |

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof.

In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

| | | SEQ |
|---|---|---|
| | | ID |
| Description | Sequence | NO: |
| Cas9 | MDKKYSIGLDIGTNSVGWAVITDEYKVPSKKFKVLGNTDRHSIKKNLIGALLFDSGETAEA | 60 |
| nickase | TRLKRTARRRYTRRKNRICYLQEIFSNEMAKVDDSFFHRLEESFLVEEDKKHERHPIFGNI | |
| Streptococcus | VDEVAYHEKYPTIYHLRKKLVDSTDKADLRLIYLALAHMIKFRGHFLIEGDLNPDNSDVDK | |
| pyogenes | LFIQLVQTYNQLFEENPINASGVDAKAILSARLSKSRRLENLIAQLPGEKKNGLFGNLIAL | |
| Q99ZW2 Cas9 | SLGLTPNFKSNFDLAEDAKLQLSKDTYDDDLDNLLAQIGDQYADLFLAAKNLSDAILLSDI | |
| with H840X, | LRVNTEITKAPLSASMIKRYDEHHQDLILLKALVRQQLPEKYKEIFFDQSKNGYAGYIDGG | |
| wherein X is | ASQEEFYKFIKPILEKMDGTEELLVKLNREDLLRKQRTFDNGSIPHQIHLGELHAILRRQE | |
| any | DFYPFLKDNREKIEKILTFRIPYYVGPLARGNSRFAWMTRKSEETITPWNFEEVVDKGASA | |
| alternate | QSFIERMTNFDKNLPNEKVLPKHSLLYEYFTVYNELTKVKYVTEGMRKPAFLSGEQKKAIV | |
| amino acid | DLLFKTNRKVTVKQLKEDYFKKIECFDSVEISGVEDRFNASLGTYHDLLKIIKDKDFLDNE | |
| | ENEDILEDIVLILTLFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDXIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIEINGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | LDATLIHQSITGLYETRIDLSQLGGD | |
| Cas9 | MDKKYSIGLDIGTNSVGWAVITDEYKVPSKKFKVLGNTDRHSIKKNLIGALLFDSGETAEA | 61 |
| nickase | TRLKRTARRRYTRRKNRICYLQEIFSNEMAKVDDSFFHRLEESFLVEEDKKHERHPIFGNI | |
| Streptococcus | VDEVAYHEKYPTIYHLRKKLVDSTDKADLRLIYLALAHMIKERGHFLIEGDLNPDNSDVDK | |
| pyogenes | LFIQLVQTYNQLFEENPINASGVDAKAILSARLSKSRRLENLIAQLPGEKKNGLFGNLIAL | |
| Q99ZW2 Cas9 | SLGLTPNFKSNFDLAEDAKLQLSKDTYDDDLDNLLAQIGDQYADLFLAAKNLSDAILLSDI | |
| with H840A, | LRVNTEITKAPLSASMIKRYDEHHQDLILLKALVRQQLPEKYKEIFFDQSKNGYAGYIDGG | |
| wherein X is | ASQEEFYKFIKPILEKMDGTEELLVKLNREDLLRKQRTFDNGSIPHQIHLGELHAILRRQE | |
| any | DFYPFLKDNREKIEKILTFRIPYYVGPLARGNSRFAWMTRKSEETITPWNFEEVVDKGASA | |
| alternate | QSFIERMTNFDKNLPNEKVLPKHSLLYEYFTVYNELTKVKYVTEGMRKPAFLSGEQKKAIV | |
| amino acid | DLLFKTNRKVTVKQLKEDYFKKIECFDSVEISGVEDRFNASLGTYHDLLKIIKDKDFLDNE | |
| | ENEDILEDIVLILILFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDAIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | 62 |
| Streptococcus | TRLKRTARRRYTRRKNRICYLQEIFSNEMAKVDDSFFHRLEESFLVEEDKKHERHPIFGNI | |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with R863X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any | ASQEEFYKFIKPILEKMDGTEELLVKLNREDLLRKQRTFDNGSIPHQIHLGELHAILRRQE | |
| alternate | DFYPFLKDNREKIEKILTFRIPYYVGPLARGNSRFAWMTRKSEETITPWNFEEVVDKGASA | |
| amino acid | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLILFEDREMIEERLKTYAHLEDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNXGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLINLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | 63 |
| Streptococcus | TRLKRTARRRYTRRKNRICYLQEIFSNEMAKVDDSFFHRLEESFLVEEDKKHERHPIFGNI | |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with R863A, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any | ASQEEFYKFIKPILEKMDGTEELLVKLNREDLLRKQRTFDNGSIPHQIHLGELHAILRRQE | |
| alternate | DFYPFLKDNREKIEKILTFRIPYYVGPLARGNSRFAWMTRKSEETITPWNFEEVVDKGASA | |
| amino acid | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDELDNE | |
| | ENEDILEDIVLTLILFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNAGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDERKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

| Description | Sequence |
|---|---|
| Cas9 nickase | DKKYSIGLDIGTNSVGWAVITDEYKVPSKKFKVLGNTDRHSIKKNLIGALLFDSGETAEATRLKRTARRRYT |
| (Met minus) | RRKNRICYLQEIFSNEMAKVDDSFFHRLEESFLVEEDKKHERHPIFGNIVDEVAYHEKYPTIYHLRKKLVDS |
| Streptococcus | TDKADLRLIYLALAHMIKFRGHFLIEGDLNPDNSDVDKLFIQLVQTYNQLFEENPINASGVDAKAILSARLS |
| pyogenes | KSRRLENLIAQLPGEKKNGLFGNLIALSLGLTPNFKSNFDLAEDAKLQLSKDTYDDDLDNLLAQIGDQYADL |
| Q99ZW2 Cas9 | FLAAKNLSDAILLSDILRVNTEITKAPLSASMIKRYDEHHQDLILLKALVRQQLPEKYKEIFFDQSKNGYAG |
| with H840X, | YIDGGASQEEFYKFIKPILEKMDGTEELLVKLNREDLLRKQRTFDNGSIPHQIHLGELHAILRRQEDFYPFL |
| wherein X is | KDNREKIEKILTFRIPYYVGPLARGNSRFAWMTRKSEETITPWNFEEVVDKGASAQSFIERMINEDKNLPNE |
| any | KVLPKHSLLYEYFTVYNELTKVKYVTEGMRKPAFLSGEQKKAIVDLLFKTNRKVTVKQLKEDYFKKIECFDS |
| alternate | VEISGVEDRFNASLGTYHDLLKIIKDKDELDNEENEDILEDIVLTLTLFEDREMIEERLKTYAHLEDDKVMK |
| amino acid | QLKRRRYTGWGRLSRKLINGIRDKQSGKTILDELKSDGFANRNEMQLIHDDSLTFKEDIQKAQVSGQGDSLH |
| | EHIANLAGSPAIKKGILQTVKVVDELVKVMGRHKPENIVIEMARENQTTQKGQKNSRERMKRIEEGIKELGS |
| | QILKEHPVENTQLQNEKLYLYYLQNGRDMYVDQELDINRLSDYDVDXIVPQSFLKDDSIDNKVLIRSDKNRG |
| | KSDNVPSEEVVKKMKNYWRQLLNAKLITQRKFDNLTKAERGGLSELDKAGFIKRQLVETRQITKHVAQILDS |
| | RMNTKYDENDKLIREVKVITLKSKLVSDFRKDFQFYKVREINNYHHAHDAYLNAVVGTALIKKYPKLESEFV |
| | YGDYKVYDVRKMIAKSEQEIGKATAKYFFYSNIMNFFKTEITLANGEIRKRPLIEINGETGEIVWDKGRDFA |
| | TVRKVLSMPQVNIVKKTEVQTGGFSKESILPKRNSDKLIARKKDWDPKKYGGFDSPTVAYSVLVVAKVEKGK |
| | SKKLKSVKELLGITIMERSSFEKNPIDFLEAKGYKEVKKDLIIKLPKYSLFELENGRKRMLASAGELQKGNE |
| | LALPSKYVNFLYLASHYEKLKGSPEDNEQKQLFVEQHKHYLDEIIEQISEFSKRVILADANLDKVLSAYNKH |
| | RDKPIREQAENIIHLFTLTNLGAPAAFKYFDTTIDRKRYTSTKEVLDATLIHQSITGLYETRIDLSQLGGD |
| | (SEQ ID NO: 64) |
| Cas9 | DKKYSIGLDIGTNSVGWAVITDEYKVPSKKFKVLGNTDRHSIKKNLIGALLFDSGETAEATRLKRTARRRYT |
| nickase | RRKNRICYLQEIFSNEMAKVDDSFFHRLEESELVEEDKKHERHPIFGNIVDEVAYHEKYPTIYHLRKKLVDS |
| (Met minus) | TDKADLRLIYLALAHMIKFRGHFLIEGDLNPDNSDVDKLFIQLVQTYNQLFEENPINASGVDAKAILSARLS |
| Streptococcus | KSRRLENLIAQLPGEKKNGLFGNLIALSLGLIPNFKSNFDLAEDAKLQLSKDTYDDDLDNLLAQIGDQYADL |
| pyogenes | FLAAKNLSDAILLSDILRVNTEITKAPLSASMIKRYDEHHQDLILLKALVRQQLPEKYKEIFFDQSKNGYAG |
| Q99ZW2 Cas9 | YIDGGASQEEFYKFIKPILEKMDGTEELLVKLNREDLLRKQRTFDNGSIPHQIHLGELHAILRRQEDFYPEL |
| with | KDNREKIEKILTFRIPYYVGPLARGNSRFAWMTRKSEETITPWNFEEVVDKGASAQSFIERMTNFDKNLPNE |
| H840A, | KVLPKHSLLYEYFTVYNELTKVKYVTEGMRKPAFLSGEQKKAIVDLLFKTNRKVTVKQLKEDYFKKIECFDS |
| wherein X is | VEISGVEDRFNASLGTYHDLLKIIKDKDELDNEENEDILEDIVLTLTLFEDREMIEERLKTYAHLFDDKVMK |
| any | QLKRRRYTGWGRLSRKLINGIRDKQSGKTILDFLKSDGFANRNFMQLIHDDSLTFKEDIQKAQVSGQGDSLH |
| alternate | EHIANLAGSPAIKKGILQTVKVVDELVKVMGRHKPENIVIEMARENQTTQKGQKNSRERMKRIEEGIKELGS |
| amino acid | QILKEHPVENTQLQNEKLYLYYLQNGRDMYVDQELDINRLSDYDVDAIVPQSFLKDDSIDNKVLTRSDKNRG |
| | KSDNVPSEEVVKKMKNYWRQLLNAKLITQRKFDNLTKAERGGLSELDKAGFIKRQLVETRQITKHVAQILDS |
| | RMNTKYDENDKLIREVKVITLKSKLVSDFRKDFQFYKVREINNYHHAHDAYLNAVVGTALIKKYPKLESEFV |
| | YGDYKVYDVRKMIAKSEQEIGKATAKYFFYSNIMNFFKTEITLANGEIRKRPLIETNGETGEIVWDKGRDFA |
| | TVRKVLSMPQVNIVKKTEVQTGGFSKESILPKRNSDKLIARKKDWDPKKYGGFDSPTVAYSVLVVAKVEKGK |
| | SKKLKSVKELLGITIMERSSFEKNPIDFLEAKGYKEVKKDLIIKLPKYSLFELENGRKRMLASAGELQKGNE |
| | LALPSKYVNFLYLASHYEKLKGSPEDNEQKQLFVEQHKHYLDEIIEQISEFSKRVILADANLDKVLSAYNKH |
| | RDKPIREQAENIIHLFTLINLGAPAAFKYFDTTIDRKRYTSTKEVLDATLIHQSITGLYETRIDLSQLGGD |
| | (SEQ ID NO: 65) |
| Cas9 | DKKYSIGLDIGTNSVGWAVITDEYKVPSKKFKVLGNTDRHSIKKNLIGALLFDSGETAEATRLKRTARRRYT |
| nickase | RRKNRICYLQEIFSNEMAKVDDSFFHRLEESELVEEDKKHERHPIFGNIVDEVAYHEKYPTIYHLRKKLVDS |
| (Met minus) | TDKADLRLIYLALAHMIKFRGHFLIEGDLNPDNSDVDKLFIQLVQTYNQLFEENPINASGVDAKAILSARLS |
| Streptococcus | KSRRLENLIAQLPGEKKNGLFGNLIALSLGLTPNFKSNFDLAEDAKLQLSKDTYDDDLDNLLAQIGDQYADL |
| pyogenes | FLAAKNLSDAILLSDILRVNTEITKAPLSASMIKRYDEHHQDLILLKALVRQQLPEKYKEIFFDQSKNGYAG |
| Q99ZW2 Cas9 | YIDGGASQEEFYKFIKPILEKMDGTEELLVKLNREDLLRKRTFDNGSIPHQIHLGELHAILRRQEDFYPFL |
| with | KDNREKIEKILTFRIPYYVGPLARGNSRFAWMTRKSEETITPWNFEEVVDKGASAQSFIERMINEDKNLPNE |
| R863X, | KVLPKHSLLYEYFTVYNELTKVKYVTEGMRKPAFLSGEQKKAIVDLLFKTNRKVTVKQLKEDYFKKIECFDS |
| wherein X is | VEISGVEDRFNASLGTYHDLLKIIKDKDELDNEENEDILEDIVLTLTLFEDREMIEERLKTYAHLEDDKVMK |
| any | QLKRRRYTGWGRLSRKLINGIRDKQSGKTILDFLKSDGFANRNFMQLIHDDSLTFKEDIQKAQVSGQGDSLH |
| alternate | EHIANLAGSPAIKKGILQTVKVVDELVKVMGRHKPENIVIEMARENQTTQKGQKNSRERMKRIEEGIKELGS |
| amino acid | QILKEHPVENTQLQNEKLYLYYLQNGRDMYVDQELDINRLSDYDVDHIVPQSFLKDDSIDNKVLIRSDKNXG |
| | KSDNVPSEEVVKKMKNYWRQLLNAKLITQRKFDNLTKAERGGLSELDKAGFIKRQLVETRQITKHVAQILDS |
| | RMNTKYDENDKLIREVKVITLKSKLVSDFRKDFQFYKVREINNYHHAHDAYLNAVVGTALIKKYPKLESEFV |
| | YGDYKVYDVRKMIAKSEQEIGKATAKYFFYSNIMNFFKTEITLANGEIRKRPLIEINGETGEIVWDKGRDFA |
| | TVRKVLSMPQVNIVKKTEVQTGGFSKESILPKRNSDKLIARKKDWDPKKYGGFDSPTVAYSVLVVAKVEKGK |
| | SKKLKSVKELLGITIMERSSFEKNPIDFLEAKGYKEVKKDLIIKLPKYSLFELENGRKRMLASAGELQKGNE |
| | LALPSKYVNFLYLASHYEKLKGSPEDNEQKQLFVEQHKHYLDEIIEQISEFSKRVILADANLDKVLSAYNKH |
| | RDKPIREQAENIIHLFTLINLGAPAAFKYFDTTIDRKRYTSTKEVLDATLIHQSITGLYETRIDLSQLGGD |
| | (SEQ ID NO: 66) |
| Cas9 | DKKYSIGLDIGTNSVGWAVITDEYKVPSKKFKVLGNTDRHSIKKNLIGALLFDSGETAEATRLKRTARRRYT |
| nickase | RRKNRICYLQEIFSNEMAKVDDSFFHRLEESFLVEEDKKHERHPIFGNIVDEVAYHEKYPTIYHLRKKLVDS |
| (Met minus) | TDKADLRLIYLALAHMIKFRGHFLIEGDLNPDNSDVDKLFIQLVQTYNQLFEENPINASGVDAKAILSARLS |
| Streptococcus | KSRRLENLIAQLPGEKKNGLFGNLIALSLGLTPNFKSNFDLAEDAKLQLSKDTYDDDLDNLLAQIGDQYADL |
| pyogenes | FLAAKNLSDAILLSDILRVNTEITKAPLSASMIKRYDEHHQDLILLKALVRQQLPEKYKEIFFDQSKNGYAG |
| Q99ZW2 Cas9 | YIDGGASQEEFYKFIKPILEKMDGTEELLVKLNREDLLRKQRTFDNGSIPHQIHLGELHAILRRQEDFYPFL |
| with R863A, | KDNREKIEKILTFRIPYYVGPLARGNSRFAWMTRKSEETITPWNFEEVVDKGASAQSFIERMINEDKNLPNE |
| wherein X is | KVLPKHSLLYEYFTVYNELTKVKYVTEGMRKPAFLSGEQKKAIVDLLFKTNRKVTVKQLKEDYFKKIECFDS |
| any | VEISGVEDRFNASLGTYHDLLKIIKDKDELDNEENEDILEDIVLTLTLFEDREMIEERLKTYAHLEDDKVMK |
| alternate | QLKRRRYTGWGRLSRKLINGIRDKQSGKTILDFLKSDGFANRNFMQLIHDDSLTFKEDIQKAQVSGQGDSLH |
| amino acid | EHIANLAGSPAIKKGILQTVKVVDELVKVMGRHKPENIVIEMARENQTTQKGQKNSRERMKRIEEGIKELGS |
| | QILKEHPVENTQLQNEKLYLYYLQNGRDMYVDQELDINRLSDYDVDHIVPQSFLKDDSIDNKVLIRSDKNAG |
| | KSDNVPSEEVVKKMKNYWRQLLNAKLITQRKFDNLTKAERGGLSELDKAGFIKRQLVETRQITKHVAQILDS |
| | RMNTKYDENDKLIREVKVITLKSKLVSDFRKDFQFYKVREINNYHHAHDAYLNAVVGTALIKKYPKLESEFV |
| | YGDYKVYDVRKMIAKSEQEIGKATAKYFFYSNIMNFFKTEITLANGEIRKRPLIEINGETGEIVWDKGRDFA |
| | TVRKVLSMPQVNIVKKTEVQTGGFSKESILPKRNSDKLIARKKDWDPKKYGGFDSPTVAYSVLVVAKVEKGK |
| | SKKLKSVKELLGITIMERSSFEKNPIDFLEAKGYKEVKKDLIIKLPKYSLFELENGRKRMLASAGELQKGNE |
| | LALPSKYVNFLYLASHYEKLKGSPEDNEQKQLFVEQHKHYLDEIIEQISEFSKRVILADANLDKVLSAYNKH |
| | RDKPIREQAENIIHLFTLINLGAPAAFKYFDTTIDRKRYTSTKEVLDATLIHQSITGLYETRIDLSQLGGD |
| | (SEQ ID NO: 67) |

### (5) Other Cas9 Variants

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 28).

In some embodiments, the disclosure also may utilize Cas9 fragments which retain their functionality and which are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the base editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

### (6) Small-Sized Cas9 Variants

In some embodiments, the base editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery.

The canonical SpCas9 protein is 1368 amino acids in length and has a predicted molecular weight of 158 kilodaltons. The term “small-sized Cas9 variant”, as used herein, refers to any Cas9 variant-naturally occurring, engineered, or otherwise—that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In various embodiments, the base editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein.

| | | SEQ |
|---|---|---|
| | | ID |
| Description | Sequence | NO: |
| SaCas9 | MGKRNYILGLDIGITSVGYGIIDYETRDVIDAGVRLFKEANVENNEGRRSKRGARRLKRRRR | 68 |
| Staphylococcus | HRIQRVKKLLFDYNLLTDHSELSGINPYEARVKGLSQKLSEEEFSAALLHLAKRRGVHNVNE | |
| aureus | VEEDTGNELSTKEQISRNSKALEEKYVAELQLERLKKDGEVRGSINRFKTSDYVKEAKQLLK | |
| 1053 AA | VQKAYHQLDQSFIDTYIDLLETRRTYYEGPGEGSPFGWKDIKEWYEMLMGHCTYFPEELRSV | |
| 123 kDa | KYAYNADLYNALNDLNNLVITRDENEKLEYYEKFQIIENVFKQKKKPTLKQIAKEILVNEED | |
| | IKGYRVTSTGKPEFTNLKVYHDIKDITARKEIIENAELLDQIAKILTIYQSSEDIQEELTNL | |
| | NSELTQEEIEQISNLKGYTGTHNLSLKAINLILDELWHINDNQIAIFNRLKLVPKKVDLSQQ | |
| | KEIPTTLVDDFILSPVVKRSFIQSIKVINAIIKKYGLPNDIIIELAREKNSKDAQKMINEMQ | |
| | KRNRQTNERIEEIIRTTGKENAKYLIEKIKLHDMQEGKCLYSLEAIPLEDLLNNPFNYEVDH | |
| | IIPRSVSFDNSFNNKVLVKQEENSKKGNRTPFQYLSSSDSKISYETFKKHILNLAKGKGRIS | |
| | KTKKEYLLEERDINRFSVQKDFINRNLVDTRYATRGLMNLLRSYFRVNNLDVKVKSINGGFT | |
| | SFLRRKWKFKKERNKGYKHHAEDALIIANADFIFKEWKKLDKAKKVMENQMFEEKQAESMPE | |
| | IETEQEYKEIFITPHQIKHIKDFKDYKYSHRVDKKPNRKLINDTLYSTRKDDKGNTLIVNNL | |
| | NGLYDKDNDKLKKLINKSPEKLLMYHHDPQTYQKLKLIMEQYGDEKNPLYKYYEETGNYLTK | |
| | YSKKDNGPVIKKIKYYGNKLNAHLDITDDYPNSRNKVVKLSLKPYRFDVYLDNGVYKFVTVK | |
| | NLDVIKKENYYEVNSKCYEEAKKLKKISNQAEFIASFYKNDLIKINGELYRVIGVNNDLLNR | |
| | IEVNMIDITYREYLENMNDKRPPHIIKTIASKTQSIKKYSTDILGNLYEVKSKKHPQIIKK | |
| NmeCas9 | MAAFKPNSINYILGLDIGIASVGWAMVEIDEEENPIRLIDLGVRVFERAEVPKTGDSLAMAR | 69 |
| N. | RLARSVRRLTRRRAHRLLRTRRLLKREGVLQAANFDENGLIKSLPNTPWQLRAAALDRKLTP | |
| meningitidis | LEWSAVLLHLIKHRGYLSQRKNEGETADKELGALLKGVAGNAHALQTGDFRTPAELALNKFE | |
| 1083 AA | KESGHIRNQRSDYSHTFSRKDLQAELILLFEKQKEFGNPHVSGGLKEGIETLLMTQRPALSG | |
| 124.5 kDa | DAVQKMLGHCTFEPAEPKAAKNTYTAERFIWLIKLNNLRILEQGSERPLTDTERATLMDEPY | |
| | RKSKLTYAQARKLLGLEDTAFFKGLRYGKDNAEASTLMEMKAYHAISRALEKEGLKDKKSPL | |
| | NLSPELQDEIGTAFSLFKTDEDITGRLKDRIQPEILEALLKHISFDKFVQISLKALRRIVPL | |
| | MEQGKRYDEACAEIYGDHYGKKNTEEKIYLPPIPADEIRNPVVLRALSQARKVINGVVRRYG | |
| | SPARIHIETAREVGKSFKDRKEIEKRQEENRKDREKAAAKFREYFPNFVGEPKSKDILKLRL | |
| | YEQQHGKCLYSGKEINLGRLNEKGYVEIDAALPESRTWDDSFNNKVLVLGSENQNKGNQTPY | |
| | EYFNGKDNSREWQEFKARVETSRFPRSKKQRILLQKFDEDGFKERNLNDTRYVNRFLCQFVA | |
| | DRMRLTGKGKKRVFASNGQITNLLRGFWGLRKVRAENDRHHALDAVVVACSTVAMQQKITRF | |
| | VRYKEMNAFDGKTIDKETGEVLHQKTHFPQPWEFFAQEVMIRVFGKPDGKPEFEEADTLEKL | |
| | RTLLAEKLSSRPEAVHEYVTPLFVSRAPNRKMSGQGHMETVKSAKRLDEGVSVLRVPLTQLK | |
| | LKDLEKMVNREREPKLYEALKARLEAHKDDPAKAFAEPFYKYDKAGNRTQQVKAVRVEQVQK | |
| | TGVWVRNHNGIADNATMVRVDVFEKGDKYYLVPIYSWQVAKGILPDRAVVQGKDEEDWQLID | |
| | DSFNFKFSLHPNDLVEVITKKARMFGYFASCHRGTGNINIRIHDLDHKIGKNGILEGIGVKT | |
| | ALSFQKYQIDELGKEIRPCRLKKRPPVR | |
| CjCas9 | MARILAFDIGISSIGWAFSENDELKDCGVRIFTKVENPKTGESLALPRRLARSARKRLARRK | 70 |
| C. jejuni | ARLNHLKHLIANEFKLNYEDYQSFDESLAKAYKGSLISPYELRFRALNELLSKQDFARVILH | |
| 984 AA | IAKRRGYDDIKNSDDKEKGAILKAIKQNEEKLANYQSVGEYLYKEYFQKFKENSKEFTNVRN | |
| 114.9 kDa | KKESYERCIAQSFLKDELKLIFKKQREFGFSFSKKFEEEVLSVAFYKRALKDFSHLVGNCSF | |
| | FTDEKRAPKNSPLAFMFVALTRIINLLNNLKNTEGILYTKDDLNALLNEVLKNGTLTYKQTK | |
| | KLLGLSDDYEFKGEKGTYFIEFKKYKEFIKALGEHNLSQDDLNEIAKDITLIKDEIKLKKAL | |
| | AKYDLNQNQIDSLSKLEFKDHLNISFKALKLVTPLMLEGKKYDEACNELNLKVAINEDKKDF | |
| | LPAFNETYYKDEVINPVVLRAIKEYRKVLNALLKKYGKVHKINIELAREVGKNHSQRAKIEK | |
| | EQNENYKAKKDAELECEKLGLKINSKNILKLRLFKEQKEFCAYSGEKIKISDLQDEKMLEID | |
| | HIYPYSRSFDDSYMNKVLVFTKQNQEKLNQTPFEAFGNDSAKWQKIEVLAKNLPTKKQKRIL | |
| | DKNYKDKEQKNFKDRNLNDTRYIARLVLNYTKDYLDFLPLSDDENTKLNDTQKGSKVHVEAK | |
| | SGMLTSALRHTWGFSAKDRNNHLHHAIDAVIIAYANNSIVKAFSDFKKEQESNSAELYAKKI | |
| | SELDYKNKRKFFEPFSGFRQKVLDKIDEIFVSKPERKKPSGALHEETFRKEEEFYQSYGGKE | |
| | GVLKALELGKIRKVNGKIVKNGDMFRVDIFKHKKINKFYAVPIYTMDFALKVLPNKAVARSK | |
| | KGEIKDWILMDENYEFCFSLYKDSLILIQTKDMQEPEFVYYNAFTSSTVSLIVSKHDNKFET | |
| | LSKNQKILFKNANEKEVIAKSIGIQNLKVFEKYIVSALGEVTKAEFRQREDFKK | |
| GeoCas9 | MRYKIGLDIGITSVGWAVMNLDIPRIEDLGVRIFDRAENPQTGESLALPRRLARSARRRLRR | 71 |
| G. | RKHRLERIRRLVIREGILTKEELDKLFEEKHEIDVWQLRVEALDRKLNNDELARVLLHLAKR | |
| stearothermo- | RGFKSNRKSERSNKENSTMLKHIEENRAILSSYRTVGEMIVKDPKFALHKRNKGENYTNTIA | |
| philus | RDDLEREIRLIFSKQREFGNMSCTEEFENEYITIWASQRPVASKDDIEKKVGFCTFEPKEKR | |
| 1087 AA | APKATYTFQSFIAWEHINKLRLISPSGARGLIDEERRLLYEQAFQKNKITYHDIRTLLHLPD | |
| 127 kDa | DTYFKGIVYDRGESRKQNENIRFLELDAYHQIRKAVDKVYGKGKSSSFLPIDEDTFGYALTL | |
| | FKDDADIHSYLRNEYEQNGKRMPNLANKVYDNELIEELLNLSFTKFGHLSLKALRSILPYME | |
| | QGEVYSSACERAGYTFTGPKKKQKTMLLPNIPPIANPVVMRALTQARKVVNAIIKKYGSPVS | |
| | IHIELARDLSQTFDERRKIKKEQDENRKKNETAIRQLMEYGLTLNPTGHDIVKFKLWSEQNG | |
| | RCAYSLQPIEIERLLEPGYVEVDHVIPYSRSLDDSYTNKVLVLTRENREKGNRIPAEYLGVG | |
| | TERWQQFETFVLINKQFSKKKRDRLLRLHYDENEETEFKNRNLNDTRYISRFFANFIREHLK | |
| | FAESDDKQKVYTVNGRVTAHLRSRWEFNKNREESDLHHAVDAVIVACTTPSDIAKVTAFYQR | |
| | REQNKELAKKTEPHFPQPWPHFADELRARLSKHPKESIKALNLGNYDDQKLESLQPVFVSRM | |
| | PKRSVTGAAHQETLRRYVGIDERSGKIQTVVKTKLSEIKLDASGHFPMYGKESDPRTYEAIR | |
| | QRLLEHNNDPKKAFQEPLYKPKKNGEPGPVIRTVKIIDTKNQVIPLNDGKTVAYNSNIVRVD | |
| | VFEKDGKYYCVPVYIMDIMKGILPNKAIEPNKPYSEWKEMTEDYTFRFSLYPNDLIRIELPR | |
| | EKTVKTAAGEEINVKDVFVYYKTIDSANGGLELISHDHRFSLRGVGSRTLKRFEKYQVDVLG | |
| | NIYKVRGEKRVGLASSAHSKPGKTIRPLQSTRD | |
| LbaCas12a | MSKLEKFTNCYSLSKTLRFKAIPVGKTQENIDNKRLLVEDEKRAEDYKGVKKLLDRYYLSFI | 72 |
| L. bacterium | NDVLHSIKLKNLNNYISLFRKKTRTEKENKELENLEINLRKEIAKAFKGNEGYKSLFKKDII | |
| 1228 AA | ETILPEFLDDKDEIALVNSENGFTTAFTGFFDNRENMFSEEAKSISIAFRCINENLTRYISN | |
| 143.9 kDa | MDIFEKVDAIFDKHEVQEIKEKILNSDYDVEDFFEGEFFNFVLTQEGIDVYNAIIGGFVTES | |
| | GEKIKGLNEYINLYNQKTKQKLPKFKPLYKQVLSDRESLSFYGEGYTSDEEVLEVERNTLNK | |
| | NSEIFSSIKKLEKLFKNFDEYSSAGIFVKNGPAISTISKDIFGEWNVIRDKWNAEYDDIHLK | |
| | KKAVVTEKYEDDRRKSFKKIGSESLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDI | |
| | LLKVDHIYDAIRNYVTQKPYSKDKFKLYFQNPQFMGGWDKDKETDYRATILRYGSKYYLAIM | |
| | DKKYAKCLQKIDKDDVNGNYEKINYKLLPGPNKMLPKVFFSKKWMAYYNPSEDIQKIYKNGT | |
| | FKKGDMFNLNDCHKLIDFFKDSISRYPKWSNAYDENFSETEKYKDIAGFYREVEEQGYKVSF | |
| | ESASKKEVDKLVEEGKLYMFQIYNKDFSDKSHGTPNLHTMYFKLLFDENNHGQIRLSGGAEL | |
| | FMRRASLKKEELVVHPANSPIANKNPDNPKKITTLSYDVYKDKRFSEDQYELHIPIAINKCP | |
| | KNIFKINTEVRVLLKHDDNPYVIGIDRGERNLLYIVVVDGKGNIVEQYSLNEIINNENGIRI | |
| | KTDYHSLLDKKEKERFEARQNWTSIENIKELKAGYISQVVHKICELVEKYDAVIALEDLNSG | |
| | FKNSRVKVEKQVYQKFEKMLIDKLNYMVDKKSNPCATGGALKGYQITNKFESFKSMSTQNGF | |
| | IFYIPAWLISKIDPSTGFVNLLKTKYTSIADSKKFISSFDRIMYVPEEDLFEFALDYKNFSR | |
| | TDADYIKKWKLYSYGNRIRIFRNPKKNNVEDWEEVCLTSAYKELFNKYGINYQQGDIRALLC | |
| | EQSDKAFYSSFMALMSLMLQMRNSITGRTDVDFLISPVKNSDGIFYDSRNYEAQENAILPKN | |
| | ADANGAYNIARKVLWAIGQFKKAEDEKLDKVKIAISNKEWLEYAQTSVKH | |
| BhCas12b | MATRSFILKIEPNEEVKKGLWKTHEVINHGIAYYMNILKLIRQEAIYEHHEQDPKNPKKVSK | 73 |
| B. hisashii | AEIQAELWDFVLKMQKCNSFTHEVDKDEVENILRELYEELVPSSVEKKGEANQLSNKFLYPL | |
| 1108 AA | VDPNSQSGKGTASSGRKPRWYNIKIAGDPSWEEEKKKWEEDKKKDPLAKILGKLAEYGLIPL | |
| 130.4 kDa | FIPYTDSNEPIVKEIKWMEKSRNQSVRRLDKDMFIQALERFLSWESWNLKVKEEYEKVEKEY | |
| | KILEERIKEDIQALKALEQYEKERQEQLLRDILNINEYRLSKRGLRGWREIIQKWLKMDENE | |
| | PSEKYLEVFKDYQRKHPREAGDYSVYEFLSKKENHFIWRNHPEYPYLYATFCEIDKKKKDAK | |
| | QQATFTLADPINHPLWVRFEERSGSNLNKYRILTEQLHTEKLKKKLTVQLDRLIYPTESGGW | |
| | EEKGKVDIVLLPSRQFYNQIFLDIEEKGKHAFTYKDESIKFPLKGTLGGARVQFDRDHLRRY | |
| | PHKVESGNVGRIYFNMTVNIEPTESPVSKSLKIHRDDFPKVVNFKPKELTEWIKDSKGKKLK | |
| | SGIESLEIGLRVMSIDLGQRQAAAASIFEVVDQKPDIEGKLFFPIKGTELYAVHRASENIKL | |
| | PGETLVKSREVLRKAREDNLKLMNQKLNFLRNVLHFQQFEDITEREKRVTKWISRQENSDVP | |
| | LVYQDELIQIRELMYKPYKDWVAFLKQLHKRLEVEIGKEVKHWRKSLSDGRKGLYGISLKNI | |
| | DEIDRTRKFLLRWSLRPTEPGEVRRLEPGQRFAIDQLNHLNALKEDRLKKMANTIIMHALGY | |
| | CYDVRKKKWQAKNPACQIILFEDLSNYNPYEERSRFENSKLMKWSRREIPRQVALQGEIYGL | |
| | QVGEVGAQFSSRFHAKTGSPGIRCSVVTKEKLQDNRFFKNLQREGRLILDKIAVLKEGDLYP | |
| | DKGGEKFISLSKDRKCVTTHADINAAQNLQKRFWIRTHGFYKVYCKAYQVDGQTVYIPESKD | |
| | QKQKIIEEFGEGYFILKDGVYEWVNAGKLKIKKGSSKQSSSELVDSDILKDSFDLASELKGE | |
| | KLMLYRDPSGNVFPSDKWMAAGVFFGKLERILISKLTNQYSISTIEDDSSKQSM | |

### (7) Cas9 Equivalents

In some embodiments, the base editors described herein can include any Cas9 equivalent. As used herein, the term “Cas9 equivalent” is a broad term that encompasses any napDNAbp protein that serves the same function as Cas9 in the present base editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The base editors described here embrace any Cas9 equivalent that would provide the same or similar function as Cas9 despite that the Cas9 equivalent may be based on a protein that arose through convergent evolution.

For example, CasX is a Cas9 equivalent that reportedly has the same function as Cas9 but which evolved through convergent evolution. Thus, the CasX protein described in Liu et al., “CasX enzymes comprises a distinct family of RNA-guided genome editors,” Nature, 2019, Vol. 566: 218-223, is contemplated to be used with the base editors described herein. In addition, any variant or modification of CasX is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), CasX, CasY, Cpf1, C2c1, C2c2, C2C3, Argonaute, Cas12a, and Cas12b. One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference. The state of the art may also now refer to Cpf1 enzymes as Casl2a.

In still other embodiments, the Cas protein may include any CRISPR associated protein, including but not limited to, Cas12a, Cas12b, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 28).

In various other embodiments, the napDNAbp can be any of the following proteins: a Cas9, a Cpf1, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a variant thereof.

Exemplary Cas9 equivalent protein sequences can include the following:

| Description | Sequence |
|---|---|
| AsCas12a | MTQFEGFTNLYQVSKTLRFELIPQGKTLKHIQEQGFIEEDKARNDHYKELKPIIDRIYKTYADQCLQLVQLDW |
| (previously | ENLSAAIDSYRKEKTEETRNALIEEQATYRNAIHDYFIGRTDNLTDAINKRHAEIYKGLFKAELFNGKVLKQL |
| known as | GTVTTTEHENALLRSFDKFTTYFSGFYENRKNVESAEDISTAIPHRIVQDNFPKFKENCHIFTRLITAVPSLR |
| Cpf1) | EHFENVKKAIGIFVSTSIEEVFSFPFYNQLLTQTQIDLYNQLLGGISREAGTEKIKGLNEVLNLAIQKNDETA |
| Acidaminococcus | HIIASLPHRFIPLFKQILSDRNTLSFILEEFKSDEEVIQSFCKYKILLRNENVLETAEALFNELNSIDLTHIF |
| sp. | ISHKKLETISSALCDHWDTLRNALYERRISELTGKITKSAKEKVQRSLKHEDINLQEIISAAGKELSEAFKQK |
| (strain | TSEILSHAHAALDQPLPTTLKKQEEKEILKSQLDSLLGLYHLLDWFAVDESNEVDPEFSARLIGIKLEMEPSL |
| BV3L6) | SFYNKARNYATKKPYSVEKFKLNFQMPTLASGWDVNKEKNNGAILFVKNGLYYLGIMPKQKGRYKALSFEPTE |
| UniProtKB | KTSEGFDKMYYDYFPDAAKMIPKCSTQLKAVTAHFQTHTTPILLSNNFIEPLEITKEIYDLNNPEKEPKKFQT |
| U2UMQ6 | AYAKKTGDQKGYREALCKWIDFTRDFLSKYTKITSIDLSSLRPSSQYKDLGEYYAELNPLLYHISFQRIAEKE |
| | IMDAVETGKLYLFQIYNKDFAKGHHGKPNLHTLYWTGLFSPENLAKTSIKLNGQAELFYRPKSRMKRMAHRLG |
| | EKMLNKKLKDQKTPIPDTLYQELYDYVNHRLSHDLSDEARALLPNVITKEVSHEIIKDRRFTSDKFFFHVPIT |
| | LNYQAANSPSKENQRVNAYLKEHPETPIIGIDRGERNLIYITVIDSTGKILEQRSLNTIQQFDYQKKLDNREK |
| | ERVAARQAWSVVGTIKDLKQGYLSQVIHEIVDLMIHYQAVVVLENLNFGFKSKRIGIAEKAVYQQFEKMLIDK |
| | LNCLVLKDYPAEKVGGVLNPYQLTDQFTSFAKMGTQSGFLFYVPAPYTSKIDPLIGFVDPFVWKTIKNHESRK |
| | HFLEGFDFLHYDVKTGDFILHFKMNRNLSFQRGLPGFMPAWDIVFEKNETQFDAKGTPFIAGKRIVPVIENHR |
| | FTGRYRDLYPANELIALLEEKGIVERDGSNILPKLLENDDSHAIDTMVALIRSVLQMRNSNAATGEDYINSPV |
| | RDLNGVCFDSRFQNPEWPMDADANGAYHIALKGQLLLNHLKESKDLKLQNGISNQDWLAYIQELRN |
| | (SEQ ID NO: 74) |
| AsCas12a | MTQFEGFTNLYQVSKTLRFELIPQGKILKHIQEQGFIEEDKARNDHYKELKPIIDRIYKTYADQCLQLVQLDW |
| nickase | ENLSAAIDSYRKEKTEETRNALIEEQATYRNAIHDYFIGRIDNLTDAINKRHAEIYKGLFKAELFNGKVLKQL |
| (e.g., | GTVTTTEHENALLRSFDKFTTYFSGFYENRKNVESAEDISTAIPHRIVQDNFPKFKENCHIFTRLITAVPSLR |
| R1226A) | EHFENVKKAIGIFVSTSIEEVFSFPFYNQLLTQTQIDLYNQLLGGISREAGTEKIKGLNEVLNLAIQKNDETA |
| | HIIASLPHRFIPLFKQILSDRNTLSFILEEFKSDEEVIQSFCKYKILLRNENVLETAEALFNELNSIDLTHIF |
| | ISHKKLETISSALCDHWDTLRNALYERRISELIGKITKSAKEKVQRSLKHEDINLQEIISAAGKELSEAFKQK |
| | TSEILSHAHAALDQPLPTTLKKQEEKEILKSQLDSLLGLYHLLDWFAVDESNEVDPEFSARLTGIKLEMEPSL |
| | SFYNKARNYATKKPYSVEKFKLNFQMPTLASGWDVNKEKNNGAILFVKNGLYYLGIMPKQKGRYKALSFEPTE |
| | KTSEGFDKMYYDYFPDAAKMIPKCSTQLKAVTAHFQTHTTPILLSNNFIEPLEITKEIYDLNNPEKEPKKFQT |
| | AYAKKTGDQKGYREALCKWIDFTRDELSKYTKITSIDLSSLRPSSQYKDLGEYYAELNPLLYHISFQRIAEKE |
| | IMDAVETGKLYLFQIYNKDFAKGHHGKPNLHTLYWTGLFSPENLAKTSIKLNGQAELFYRPKSRMKRMAHRLG |
| | EKMLNKKLKDQKTPIPDTLYQELYDYVNHRLSHDLSDEARALLPNVITKEVSHEIIKDRRFTSDKFFFHVPIT |
| | LNYQAANSPSKFNQRVNAYLKEHPETPIIGIDRGERNLIYITVIDSTGKILEQRSLNTIQQFDYQKKLDNREK |
| | ERVAARQAWSVVGTIKDLKQGYLSQVIHEIVDLMIHYQAVVVLENLNFGFKSKRIGIAEKAVYQQFEKMLIDK |
| | LNCLVLKDYPAEKVGGVLNPYQLTDQFTSFAKMGTQSGFLFYVPAPYTSKIDPLIGFVDPFVWKTIKNHESRK |
| | HFLEGFDFLHYDVKTGDFILHFKMNRNLSFQRGLPGEMPAWDIVFEKNETQFDAKGTPFIAGKRIVPVIENHR |
| | FTGRYRDLYPANELIALLEEKGIVERDGSNILPKLLENDDSHAIDTMVALIRSVLQMANSNAATGEDYINSPV |
| | RDLNGVCFDSRFQNPEWPMDADANGAYHIALKGQLLLNHLKESKDLKLQNGISNQDWLAYIQELRN (SEQ |
| | ID NO: 75) |
| LbCas12a | MNYKTGLEDFIGKESLSKTLRNALIPTESTKIHMEEMGVIRDDELRAEKQQELKEIMDDYYRTFIEEKLGQIQ |
| (previously | GIQWNSLFQKMEETMEDISVRKDLDKIQNEKRKEICCYFTSDKRFKDLFNAKLITDILPNFIKDNKEYTEEEK |
| known as Cpf1) | AEKEQTRVLFQRFATAFTNYFNQRRNNFSEDNISTAISFRIVNENSEIHLQNMRAFQRIEQQYPEEVCGMEEE |
| Lachnospiraceae | YKDMLQEWQMKHIYSVDFYDRELTQPGIEYYNGICGKINEHMNQFCQKNRINKNDFRMKKLHKQILCKKSSYY |
| bacterium | EIPFRFESDQEVYDALNEFIKTMKKKEIIRRCVHLGQECDDYDLGKIYISSNKYEQISNALYGSWDTIRKCIK |
| GAM79 | EEYMDALPGKGEKKEEKAEAAAKKEEYRSIADIDKIISLYGSEMDRTISAKKCITEICDMAGQISIDPLVCNS |
| Ref Seq. | DIKLLQNKEKTTEIKTILDSFLHVYQWGQTFIVSDIIEKDSYFYSELEDVLEDFEGITTLYNHVRSYVTQKPY |
| WP_119623382.1 | STVKFKLHFGSPTLANGWSQSKEYDNNAILLMRDQKFYLGIFNVRNKPDKQIIKGHEKEEKGDYKKMIYNLLP |
| | GPSKMLPKVFITSRSGQETYKPSKHILDGYNEKRHIKSSPKFDLGYCWDLIDYYKECIHKHPDWKNYDFHFSD |
| | TKDYEDISGFYREVEMQGYQIKWTYISADEIQKLDEKGQIFLFQIYNKDFSVHSTGKDNLHTMYLKNLFSEEN |
| | LKDIVLKLNGEAELFFRKASIKTPIVHKKGSVLVNRSYTQTVGNKEIRVSIPEEYYTEIYNYLNHIGKGKLSS |
| | EAQRYLDEGKIKSFTATKDIVKNYRYCCDHYFLHLPITINFKAKSDVAVNERTLAYIAKKEDIHIIGIDRGER |
| | NLLYISVVDVHGNIREQRSFNIVNGYDYQQKLKDREKSRDAARKNWEEIEKIKELKEGYLSMVIHYIAQLVVK |
| | YNAVVAMEDLNYGFKTGRFKVERQVYQKFETMLIEKLHYLVFKDREVCEEGGVLRGYQLTYIPESLKKVGKQC |
| | GFIFYVPAGYTSKIDPTTGFVNLFSFKNLTNRESRQDFVGKFDEIRYDRDKKMFEFSFDYNNYIKKGTILAST |
| | KWKVYTNGTRLKRIVVNGKYTSQSMEVELTDAMEKMLQRAGIEYHDGKDLKGQIVEKGIEAEIIDIFRLTVQM |
| | RNSRSESEDREYDRLISPVLNDKGEFFDTATADKILPQDADANGAYCIALKGLYEVKQIKENWKENEQFPRNK |
| | LVQDNKTWEDEMQKKRYL (SEQ ID NO: 76) |
| PcCas12a - | MAKNFEDFKRLYSLSKTLRFEAKPIGATLDNIVKSGLLDEDEHRAASYVKVKKLIDEYHKVFIDRVLDDGCLP |
| previously | LENKGNNNSLAEYYESYVSRAQDEDAKKKFKEIQQNLRSVIAKKLTEDKAYANLFGNKLIESYKDKEDKKKII |
| known at | DSDLIQFINTAESTQLDSMSQDEAKELVKEFWGFVTYFYGFFDNRKNMYTAEEKSTGIAYRLVNENLPKFIDN |
| Cpf1 | IEAFNRAITRPEIQENMGVLYSDESEYLNVESIQEMFQLDYYNMLLTQKQIDVYNAIIGGKTDDEHDVKIKGI |
| Prevotella | NEYINLYNQQHKDDKLPKLKALFKQILSDRNAISWLPEEFNSDQEVLNAIKDCYERLAENVLGDKVLKSLLGS |
| copri | LADYSLDGIFIRNDLQLTDISQKMFGNWGVIQNAIMQNIKRVAPARKHKESEEDYEKRIAGIFKKADSFSISY |
| Ref Seq. | INDCLNEADPNNAYFVENYFATFGAVNTPTMQRENLFALVQNAYTEVAALLHSDYPTVKHLAQDKANVSKIKA |
| WP_119227726.1 | LLDAIKSLQHFVKPLLGKGDESDKDERFYGELASLWAELDTVTPLYNMIRNYMTRKPYSQKKIKLNFENPQLL |
| | GGWDANKEKDYATIILRRNGLYYLAIMDKDSRKLLGKAMPSDGECYEKMVYKFFKDVTTMIPKCSTQLKDVQA |
| | YFKVNTDDYVLNSKAFNKPLTITKEVEDLNNVLYGKYKKFQKGYLTATGDNVGYTHAVNVWIKFCMDELNSYD |
| | STCIYDFSSLKPESYLSLDAFYQDANLLLYKLSFARASVSYINQLVEEGKMYLFQIYNKDFSEYSKGTPNMHT |
| | LYWKALFDERNLADVVYKLNGQAEMFYRKKSIENTHPTHPANHPILNKNKDNKKKESLFDYDLIKDRRYTVDK |
| | FMFHVPITMNFKSVGSENINQDVKAYLRHADDMHIIGIDRGERHLLYLVVIDLQGNIKEQYSLNEIVNEYNGN |
| | TYHTNYHDLLDVREEERLKARQSWQTIENIKELKEGYLSQVIHKITQLMVRYHAIVVLEDLSKGEMRSRQKVE |
| | KQVYQKFEKMLIDKLNYLVDKKTDVSTPGGLLNAYQLTCKSDSSQKLGKQSGELFYIPAWNTSKIDPVTGFVN |
| | LLDTHSLNSKEKIKAFFSKFDAIRYNKDKKWFEFNLDYDKFGKKAEDTRIKWTLCTRGMRIDTERNKEKNSQW |
| | DNQEVDLTTEMKSLLEHYYIDIHGNLKDAISAQTDKAFFTGLLHILKLTLQMRNSITGTETDYLVSPVADENG |
| | IFYDSRSCGNQLPENADANGAYNIARKGLMLIEQIKNAEDLNNVKFDISNKAWINFAQQKPYKNG (SEQ ID |
| | NO: 77) |
| ErCas12a - | MFSAKLISDILPEFVIHNNNYSASEKEEKTQVIKLESRFATSFKDYFKNRANCESANDISSSSCHRIVNDNAE |
| previously | IFFSNALVYRRIVKNLSNDDINKISGDMKDSLKEMSLEEIYSYEKYGEFITQEGISFYNDICGKVNLEMNLYC |
| known at | QKNKENKNLYKLRKLHKQILCIADTSYEVPYKFESDEEVYQSVNGELDNISSKHIVERLRKIGENYNGYNLDK |
| Cpf1 | IYIVSKFYESVSQKTYRDWETINTALEIHYNNILPGNGKSKADKVKKAVKNDLQKSITEINELVSNYKLCPDD |
| Eubacterium | NIKAETYIHEISHILNNFEAQELKYNPEIHLVESELKASELKNVLDVIMNAFHWCSVFMTEELVDKDNNFYAE |
| rectale | LEEIYDEIYPVISLYNLVRNYVTQKPYSTKKIKLNFGIPTLADGWSKSKEYSNNAIILMRDNLYYLGIFNAKN |
| Ref Seq. | KPDKKIIEGNTSENKGDYKKMIYNLLPGPNKMIPKVFLSSKTGVETYKPSAYILEGYKQNKHLKSSKDEDITE |
| WP_119223642.1 | CHDLIDYFKNCIAIHPEWKNFGFDFSDTSTYEDISGFYREVELQGYKIDWTYISEKDIDLLQEKGQLYLFQIY |
| | NKDFSKKSSGNDNLHTMYLKNLFSEENLKDIVLKLNGEAEIFFRKSSIKNPIIHKKGSILVNRTYEAEEKDQF |
| | GNIQIVRKTIPENIYQELYKYFNDKSDKELSDEAAKLKNVVGHHEAATNIVKDYRYTYDKYFLHMPITINFKA |
| | NKTSFINDRILQYIAKEKDLHVIGIDRGERNLIYVSVIDTCGNIVEQKSFNIVNGYDYQIKLKQQEGARQIAR |
| | KEWKEIGKIKEIKEGYLSLVIHEISKMVIKYNAIIAMEDLSYGFKKGRFKVERQVYQKFETMLINKLNYLVEK |
| | DISITENGGLLKGYQLTYIPDKLKNVGHQCGCIFYVPAAYTSKIDPTTGFVNIFKFKDLTVDAKREFIKKFDS |
| | IRYDSDKNLFCFTFDYNNFITQNTVMSKSSWSVYTYGVRIKRRFVNGRFSNESDTIDITKDMEKTLEMTDINW |
| | RDGHDLRQDIIDYEIVQHIFEIFKLTVQMRNSLSELEDRDYDRLISPVLNENNIFYDSAKAGDALPKDADANG |
| | AYCIALKGLYEIKQITENWKEDGKFSRDKLKISNKDWEDFIQNKRYL (SEQ ID NO: 78) |
| CsCas12a - | MNYKTGLEDFIGKESLSKTLRNALIPTESTKIHMEEMGVIRDDELRAEKQQELKEIMDDYYRAFIEEKLGQIQ |
| previously | GIQWNSLFQKMEETMEDISVRKDLDKIQNEKRKEICCYFTSDKRFKDLFNAKLITDILPNFIKDNKEYTEEEK |
| known at | AEKEQTRVLFQRFATAFTNYFNQRRNNFSEDNISTAISFRIVNENSEIHLQNMRAFQRIEQQYPEEVCGMEEE |
| Cpf1 | YKDMLQEWQMKHIYLVDFYDRVLTQPGIEYYNGICGKINEHMNQFCQKNRINKNDFRMKKLHKQILCKKSSYY |
| Clostridium | EIPFRFESDQEVYDALNEFIKIMKEKEIICRCVHLGQKCDDYDLGKIYISSNKYEQISNALYGSWDTIRKCIK |
| sp. | EEYMDALPGKGEKKEEKAEAAAKKEEYRSIADIDKIISLYGSEMDRTISAKKCITEICDMAGQISTDPLVCNS |
| AF34-10BH | DIKLLQNKEKTTEIKTILDSFLHVYQWGQTFIVSDIIEKDSYFYSELEDVLEDFEGITTLYNHVRSYVTQKPY |
| Ref Seq: | STVKFKLHFGSPTLANGWSQSKEYDNNAILLMRDQKFYLGIFNVRNKPDKQIIKGHEKEEKGDYKKMIYNLLP |
| WP_118538418.1 | GPSKMLPKVFITSRSGQETYKPSKHILDGYNEKRHIKSSPKFDLGYCWDLIDYYKECIHKHPDWKNYDFHFSD |
| | TKDYEDISGFYREVEMQGYQIKWTYISADEIQKLDEKGQIFLFQIYNKDFSVHSTGKDNLHTMYLKNLFSEEN |
| | LKDIVLKLNGEAELFFRKASIKTPVVHKKGSVLVNRSYTQTVGDKEIRVSIPEEYYTEIYNYLNHIGRGKLST |
| | EAQRYLEERKIKSFTATKDIVKNYRYCCDHYFLHLPITINFKAKSDIAVNERTLAYIAKKEDIHIIGIDRGER |
| | NLLYISVVDVHGNIREQRSFNIVNGYDYQQKLKDREKSRDAARKNWEEIEKIKELKEGYLSMVIHYIAQLVVK |
| | YNAVVAMEDLNYGFKTGRFKVERQVYQKFETMLIEKLHYLVFKDREVCEEGGVLRGYQLTYIPESLKKVGKQC |
| | GFIFYVPAGYTSKIDPTTGFVNLFSFKNLINRESRQDFVGKFDEIRYDRDKKMFEFSFDYNNYIKKGTMLAST |
| | KWKVYINGTRLKRIVVNGKYTSQSMEVELTDAMEKMLQRAGIEYHDGKDLKGQIVEKGIEAEIIDIFRLTVQM |
| | RNSRSESEDREYDRLISPVLNDKGEFFDTATADKTLPQDADANGAYCIALKGLYEVKQIKENWKENEQFPRNK |
| | LVQDNKTWFDFMQKKRYL (SEQ ID NO: 79) |
| BhCas12b | MATRSFILKIEPNEEVKKGLWKTHEVLNHGIAYYMNILKLIRQEAIYEHHEQDPKNPKKVSKAEIQAELWDFV |
| Bacillus | LKMQKCNSFTHEVDKDEVENILRELYEELVPSSVEKKGEANQLSNKFLYPLVDPNSQSGKGTASSGRKPRWYN |
| hisashii | LKIAGDPSWEEEKKKWEEDKKKDPLAKILGKLAEYGLIPLFIPYTDSNEPIVKEIKWMEKSRNQSVRRLDKDM |
| Ref Seq. | FIQALERFLSWESWNLKVKEEYEKVEKEYKTLEERIKEDIQALKALEQYEKERQEQLLRDTLNTNEYRLSKRG |
| WP_095142515.1 | LRGWREIIQKWLKMDENEPSEKYLEVFKDYQRKHPREAGDYSVYEFLSKKENHFIWRNHPEYPYLYATFCEID |
| | KKKKDAKQQATFTLADPINHPLWVRFEERSGSNLNKYRILTEQLHTEKLKKKLTVQLDRLIYPTESGGWEEKG |
| | KVDIVLLPSRQFYNQIFLDIEEKGKHAFTYKDESIKFPLKGTLGGARVQFDRDHLRRYPHKVESGNVGRIYFN |
| | MTVNIEPTESPVSKSLKIHRDDFPKVVNFKPKELTEWIKDSKGKKLKSGIESLEIGLRVMSIDLGQRQAAAAS |
| | IFEVVDQKPDIEGKLFFPIKGTELYAVHRASFNIKLPGETLVKSREVLRKAREDNLKLMNQKLNFLRNVLHFQ |
| | QFEDITEREKRVTKWISRQENSDVPLVYQDELIQIRELMYKPYKDWVAFLKQLHKRLEVEIGKEVKHWRKSLS |
| | DGRKGLYGISLKNIDEIDRTRKELLRWSLRPTEPGEVRRLEPGQRFAIDQLNHLNALKEDRLKKMANTIIMHA |
| | LGYCYDVRKKKWQAKNPACQIILFEDLSNYNPYEERSRFENSKLMKWSRREIPRQVALQGEIYGLQVGEVGAQ |
| | FSSRFHAKTGSPGIRCSVVTKEKLQDNRFFKNLQREGRLILDKIAVLKEGDLYPDKGGEKFISLSKDRKCVTT |
| | HADINAAQNLQKRFWIRTHGFYKVYCKAYQVDGQTVYIPESKDQKQKIIEEFGEGYFILKDGVYEWVNAGKLK |
| | IKKGSSKQSSSELVDSDILKDSFDLASELKGEKLMLYRDPSGNVFPSDKWMAAGVFFGKLERILISKLINQYS |
| | ISTIEDDSSKQSM (SEQ ID NO: 80) |
| ThCas12b | MSEKTTQRAYTLRLNRASGECAVCQNNSCDCWHDALWATHKAVNRGAKAFGDWLLTLRGGLCHTLVEMEVPAK |
| Thermomonas | GNNPPQRPTDQERRDRRVLLALSWLSVEDEHGAPKEFIVATGRDSADDRAKKVEEKLREILEKRDFQEHEIDA |
| hydrothermalis | WLQDCGPSLKAHIREDAVWVNRRALFDAAVERIKTLTWEEAWDFLEPFFGTQYFAGIGDGKDKDDAEGPARQG |
| Ref Seq. | EKAKDLVQKAGQWLSARFGIGTGADFMSMAEAYEKIAKWASQAQNGDNGKATIEKLACALRPSEPPTLDTVLK |
| WP_072754838 | CISGPGHKSATREYLKTLDKKSTVTQEDLNQLRKLADEDARNCRKKVGKKGKKPWADEVLKDVENSCELTYLQ |
| | DNSPARHREFSVMLDHAARRVSMAHSWIKKAEQRRRQFESDAQKLKNLQERAPSAVEWLDRFCESRSMTTGAN |
| | TGSGYRIRKRAIEGWSYVVQAWAEASCDTEDKRIAAARKVQADPEIEKFGDIQLFEALAADEAICVWRDQEGT |
| | QNPSILIDYVTGKTAEHNQKRFKVPAYRHPDELRHPVFCDFGNSRWSIQFAIHKEIRDRDKGAKQDTRQLQNR |
| | HGLKMRLWNGRSMTDVNLHWSSKRLTADLALDQNPNPNPTEVTRADRLGRAASSAFDHVKIKNVFNEKEWNGR |
| | LQAPRAELDRIAKLEEQGKTEQAEKLRKRLRWYVSFSPCLSPSGPFIVYAGQHNIQPKRSGQYAPHAQANKGR |
| | ARLAQLILSRLPDLRILSVDLGHRFAAACAVWETLSSDAFRREIQGLNVLAGGSGEGDLFLHVEMTGDDGKRR |
| | TVVYRRIGPDQLLDNTPHPAPWARLDRQFLIKLQGEDEGVREASNEELWTVHKLEVEVGRTVPLIDRMVRSGF |
| | GKTEKQKERLKKLRELGWISAMPNEPSAETDEKEGEIRSISRSVDELMSSALGTLRLALKRHGNRARIAFAMT |
| | ADYKPMPGGQKYYFHEAKEASKNDDETKRRDNQIEFLQDALSLWHDLFSSPDWEDNEAKKLWQNHIATLPNYQ |
| | TPEEISAELKRVERNKKRKENRDKLRTAAKALAENDQLRQHLHDTWKERWESDDQQWKERLRSLKDWIFPRGK |
| | AEDNPSIRHVGGLSITRINTISGLYQILKAFKMRPEPDDLRKNIPQKGDDELENFNRRLLEARDRLREQRVKQ |
| | LASRIIEAALGVGRIKIPKNGKLPKRPRTTVDTPCHAVVIESLKTYRPDDLRTRRENRQLMQWSSAKVRKYLK |
| | EGCELYGLHFLEVPANYTSRQCSRTGLPGIRCDDVPTGDFLKAPWWRRAINTAREKNGGDAKDRFLVDLYDHL |
| | NNLQSKGEALPATVRVPRQGGNLFIAGAQLDDINKERRAIQADLNAAANIGLRALLDPDWRGRWWYVPCKDGT |
| | SEPALDRIEGSTAFNDVRSLPTGDNSSRRAPREIENLWRDPSGDSLESGTWSPTRAYWDTVQSRVIELLRRHA |
| | GLPTS (SEQ ID NO: 81) |
| LsCas12b | MSIRSFKLKLKTKSGVNAEQLRRGLWRTHQLINDGIAYYMNWLVLLRQEDLFIRNKETNEIEKRSKEEIQAVL |
| Laceyella | LERVHKQQQRNQWSGEVDEQTLLQALRQLYEEIVPSVIGKSGNASLKARFFLGPLVDPNNKTTKDVSKSGPTP |
| sacchari | KWKKMKDAGDPNWVQEYEKYMAERQTLVRLEEMGLIPLFPMYTDEVGDIHWLPQASGYTRTWDRDMFQQAIER |
| WP_132221894.1 | LLSWESWNRRVRERRAQFEKKTHDFASRFSESDVQWMNKLREYEAQQEKSLEENAFAPNEPYALTKKALRGWE |
| | RVYHSWMRLDSAASEEAYWQEVATCQTAMRGEFGDPAIYQFLAQKENHDIWRGYPERVIDFAELNHLQRELRR |
| | AKEDATFTLPDSVDHPLWVRYEAPGGTNIHGYDLVQDTKRNLTLILDKFILPDENGSWHEVKKVPFSLAKSKQ |
| | FHRQVWLQEEQKQKKREVVFYDYSTNLPHLGTLAGAKLQWDRNFLNKRTQQQIEETGEIGKVFFNISVDVRPA |
| | VEVKNGRLQNGLGKALTVLTHPDGTKIVTGWKAEQLEKWVGESGRVSSLGLDSLSEGLRVMSIDLGQRTSATV |
| | SVFEITKEAPDNPYKFFYQLEGTEMFAVHQRSFLLALPGENPPQKIKQMREIRWKERNRIKQQVDQLSAILRL |
| | HKKVNEDERIQAIDKLLQKVASWQLNEEIATAWNQALSQLYSKAKENDLQWNQAIKNAHHQLEPVVGKQISLW |
| | RKDLSTGRQGIAGLSLWSIEELEATKKLLTRWSKRSREPGVVKRIERFETFAKQIQHHINQVKENRLKQLANL |
| | IVMTALGYKYDQEQKKWIEVYPACQVVLFENLRSYRFSFERSRRENKKLMEWSHRSIPKLVQMQGELFGLQVA |
| | DVYAAYSSRYHGRTGAPGIRCHALTEADLRNETNIIHELIEAGFIKEEHRPYLQQGDLVPWSGGELFATLQKP |
| | YDNPRILTLHADINAAQNIQKRFWHPSMWFRVNCESVMEGEIVTYVPKNKTVHKKQGKTFRFVKVEGSDVYEW |
| | AKWSKNRNKNTFSSITERKPPSSMILFRDPSGTFFKEQEWVEQKTFWGKVQSMIQAYMKKTIVQRMEE (SEQ |
| | ID NO: 82) |
| DtCas12b | MVLGRKDDTAELRRALWTTHEHVNLAVAEVERVLLRCRGRSYWILDRRGDPVHVPESQVAEDALAMAREAQRR |
| Dsulfonatronum | NGWPVVGEDEEILLALRYLYEQIVPSCLLDDLGKPLKGDAQKIGTNYAGPLFDSDTCRRDEGKDVACCGPFHE |
| thiodismutans | VAGKYLGALPEWATPISKQEFDGKDASHLRFKATGGDDAFFRVSIEKANAWYEDPANQDALKNKAYNKDDWKK |
| WP_031386437 | EKDKGISSWAVKYIQKQLQLGQDPRTEVRRKLWLELGLLPLFIPVEDKIMVGNLWNRLAVRLALAHLLSWESW |
| | NHRAVQDQALARAKRDELAALFLGMEDGFAGLREYELRRNESIKQHAFEPVDRPYVVSGRALRSWTRVREEWL |
| | RHGDTQESRKNICNRLQDRLRGKFGDPDVFHWLAEDGQEALWKERDCVTSFSLLNDADGLLEKRKGYALMTFA |
| | DARLHPRWAMYEAPGGSNLRTYQIRKTENGLWADVVLLSPRNESAAVEEKTENVRLAPSGQLSNVSFDQIQKG |
| | SKMVGRCRYQSANQQFEGLLGGAEILFDRKRIANEQHGATDLASKPGHVWFKLTLDVRPQAPQGWLDGKGRPA |
| | LPPEAKHFKTALSNKSKFADQVRPGLRVLSVDLGVRSFAACSVFELVRGGPDQGTYFPAADGRTVDDPEKLWA |
| | KHERSFKITLPGENPSRKEEIARRAAMEELRSLNGDIRRLKAILRLSVLQEDDPRTEHLRLFMEAIVDDPAKS |
| | ALNAELFKGFGDDRFRSTPDLWKQHCHFFHDKAEKVVAERFSRWRTETRPKSSSWQDWRERRGYAGGKSYWAV |
| | TYLEAVRGLILRWNMRGRTYGEVNRQDKKQFGTVASALLHHINQLKEDRIKTGADMIIQAARGFVPRKNGAGW |
| | VQVHEPCRLILFEDLARYRFRIDRSRRENSRLMRWSHREIVNEVGMQGELYGLHVDTTEAGESSRYLASSGAP |
| | GVRCRHLVEEDFHDGLPGMHLVGELDWLLPKDKDRTANEARRLLGGMVRPGMLVPWDGGELFATLNAASQLHV |
| | IHADINAAQNLQRRFWGRCGEAIRIVCNQLSVDGSTRYEMAKAPKARLLGALQQLKNGDAPFHLTSIPNSQKP |
| | ENSYVMTPTNAGKKYRAGPGEKSSGEEDELALDIVEQAEELAQGRKTFFRDPSGVFFAPDRWLPSEIYWSRIR |
| | RRIWQVTLERNSSGRQERAEMDEMPY (SEQ ID NO: 83) |

The base editors described herein may also comprise Cas12a/Cpf1 (dCpf1) variants that may be used as a guide nucleotide sequence-programmable DNA-binding protein domain. The Cas12a/Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity.

(8) Cas9 Equivalents with Expanded PAM Sequence

In some embodiments, the napDNAbp is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 Jul;34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the napDNAbp is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other argonaute proteins may be used, and are within the scope of this disclosure.

In some embodiments, the napDNAbp is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13;538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19;65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15;167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the napDNAbp may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein.

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “editing window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

For example, a napDNAbp domain with altered PAM specificity, such as a domain with at least 80%, at least 85%, at least 90%, at least 95%, or at least 99% sequence identity with wild type Francisella novicida Cpf1 (SEQ ID NO: 84) (D91′7, E1006, and D1255), which has the following amino acid sequence:

(SEQ ID NO: 84)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKENTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLEDDLKAQKLDLSKIYFKNDKSLT
DLSQQVEDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQINNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKENDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKIGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINERNSDKNHNWDTREVYPTKELEKLLKDYSIEYGHGECIKAAICGESD
KKFFAKLISVLNTILQMRNSKTGTELDYLISPVADVNGNFFDSRQAPKNM
PQDADANGAYHIGLKGLMLLGRIKNNQEGKKLNLVIKNEEYFEFVQNRNN

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 85), which has the following amino acid sequence:

(SEQ ID NO: 85)
MKYKIGLDIGITSIGWAVINLDIPRIEDLGVRIFDRAENPKTGESLALPR
RLARSARRRLRRRKHRLERIRRLFVREGILTKEELNKLFEKKHEIDVWQL
RVEALDRKLNNDELARILLHLAKRRGERSNRKSERINKENSTMLKHIEEN
QSILSSYRTVAEMVVKDPKFSLHKRNKEDNYTNTVARDDLEREIKLIFAK
QREYGNIVCTEAFEHEYISIWASQRPFASKDDIEKKVGFCTFEPKEKRAP
KATYTFQSFTVWEHINKLRLVSPGGIRALTDDERRLIYKQAFHKNKITFH
DVRILLNLPDDTRFKGLLYDRNTTLKENEKVRFLELGAYHKIRKAIDSVY
GKGAAKSFRPIDFDTFGYALTMEKDDTDIRSYLRNEYEQNGKRMENLADK
VYDEELIEELLNLSFSKFGHLSLKALRNILPYMEQGEVYSTACERAGYTF
TGPKKKQKTVLLPNIPPIANPVVMRALTQARKVVNAIIKKYGSPVSIHIE
LARELSQSFDERRKMQKEQEGNRKKNETAIRQLVEYGLILNPTGLDIVKF
KLWSEQNGKCAYSLQPIEIERLLEPGYTEVDHVIPYSRSLDDSYTNKVLV
LIKENREKGNRTPAEYLGLGSERWQQFETFVLINKQFSKKKRDRLLRLHY
DENEENEFKNRNLNDTRYISRFLANFIREHLKFADSDDKQKVYTVNGRIT
AHLRSRWNENKNREESNLHHAVDAAIVACTTPSDIARVTAFYQRREQNKE
LSKKTDPQFPQPWPHFADELQARLSKNPKESIKALNLGNYDNEKLESLQP
VFVSRMPKRSITGAAHQETLRRYIGIDERSGKIQTVVKKKLSEIQLDKTG
HFPMYGKESDPRTYEAIRQRLLEHNNDPKKAFQEPLYKPKKNGELGPIIR
TIKIIDTTNQVIPLNDGKTVAYNSNIVRVDVFEKDGKYYCVPIYTIDMMK
GILPNKAIEPNKPYSEWKEMTEDYTFRFSLYPNDLIRIEFPREKTIKTAV
GEEIKIKDLFAYYQTIDSSNGGLSLVSHDNNFSLRSIGSRTLKRFEKYQV
DVLGNIYKVRGEKRVGVASSSHSKAGETIRPL

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 63.

The disclosed fusion proteins may comprise a napDNAbp domain having at least 80%, at least 85%, at least 90%, at least 95%, or at least 99% sequence identity with wild type Natronobacterium gregoryi Argonaute (SEQ ID NO: 86), which has the following amino acid sequence:

(SEQ ID NO: 86)
MTVIDLDSTTTADELTSGHTYDISVTLIGVYDNTDEQHPRMS
LAFEQDNGERRYITLWKNTTPKDVFTYDYATGSTYIFTNID
YEVKDGYENLTATYQTTVENATAQEVGTTDEDETFAGGEPLD
HHLDDALNETPDDAETESDSGHVMTSFASRDQLPEWTLHTY
TLTATDGAKTDTEYARRTLAYTVRQELYTDHDAAPVATDGLM
LLTPEPLGETPLDLDCGVRVEADETRTLDYTTAKDRLLARE
LVEEGLKRSLWDDYLVRGIDEVLSKEPVLTCDEFDLHERYDL
SVEVGHSGRAYLHINFRHRFVPKLTLADIDDDNIYPGLRVK
TTYRPRRGHIVWGLRDECATDSLNTLGNQSVVAYHRNNQTPI
NTDLLDAIEAADRRVVETRROGHGDDAVSFPQELLAVEPNT
HQIKQFASDGFHQQARSKTRLSASRCSEKAQAFAERLDPVRL
NGSTVEFSSEFFTGNNEQQLRLLYENGESVLTFRDGARGAH
PDETFSKGIVNPPESFEVAVVLPEQQADTCKAQWDTMADLLN
QAGAPPTRSETVQYDAFSSPESISLNVAGAIDPSEVDAAFV
VLPPDQEGFADLASPTETYDELKKALANMGIYSQMAYFDRFR
DAKIFYTRNVALGLLAAAGGVAFTTEHAMPGDADMFIGIDV
SRSYPEDGASGQINIAATATAVYKDGTILGHSSTRPQLGEKL
QSTDVRDIMKNAILGYQQVTGESPTHIVIHRDGFMNEDLDP
ATEFLNEQGVEYDIVEIRKQPQTRLLAVSDVQYDTPVKSIAA
INQNEPRATVATFGAPEYLATRDGGGLPRPIQIERVAGETD
IETLTRQVYLLSQSHIQVHNSTARLPITTAYADQASTHATKG
YLVQTGAFESNVGFL

### (9) Cas9 Circular Permutants

In various embodiments, the base editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The instant disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure:
N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB-Q99ZW2 (CAS9_STRP1) (numbering is based on the amino acid position in SEQ ID NO: 28)):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus; or
N-terminus-[10-1368]-[optional linker]-[1-9]-C-terminus, or the corresponding circular permutants of other Cas9 proteins (including other Cas9 orthologs, variants, etc).

In particular embodiments, the circular permuant Cas9 has the following structure (based on S. pyogenes Cas9 (1368 amino acids of UniProtKB-Q99ZW2 (CAS9_STRP1) (numbering is based on the amino acid position in SEQ ID NO: 28):
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In still other embodiments, the circular permuant Cas9 has the following structure (based on S. pyogenes Cas9 (1368 amino acids of UniProtKB-Q99ZW2 (CAS9_STRP1) (numbering is based on the amino acid position in SEQ ID NO: 28):
N-terminus-[103-1368]-[optional linker]-[1-102]-C-terminus;
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., any one of SEQ ID NO: 28, 8, 10, 12-26). The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., of SEQ ID NO: 28, 8, 10, 12-26).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus, includes or corresponds to the C-terminal 30% or less of the amino acids of a Cas9 (e.g., amino acids 1012-1368 of SEQ ID NO: 28). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., the Cas9 of SEQ ID NO: 28). In some embodiments, the C-terminal fragment that is rearranged to the N-terminus, includes or corresponds to the C-terminal 410 residues or less of a Cas9 (e.g., the Cas9 of SEQ ID NO: 28). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 5). In some embodiments, the C-terminal portion that is rearranged to the N-terminus, includes or corresponds to the C-terminal 357, 341, 328, 120, or 69 residues of a Cas9 (e.g., the Cas9 of SEQ ID NO: 28).

In other embodiments, circular permutant Cas9 variants may be defined as a topological rearrangement of a Cas9 primary structure based on the following method, which is based on S. pyogenes Cas9 of SEQ ID NO: 28: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to preceed the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 28) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 28, but may be implemented to make CP variants in any Cas9 sequence, either at CP sites that correspond to these positions, or at other CP sites entireley. This description is not meant to limit the specific CP sites in any way. Virtually any CP site may be used to form a CP-Cas9 variant.

Exemplary CP-Cas9 amino acid sequences, based on the Cas9 of SEQ ID NO: 28, are provided below in which linker sequences are indicated by underlining and optional methionine (M) residues are indicated in bold. It should be appreciated that the disclosure provides CP-Cas9 sequences that do not include a linker sequence or that include different linker sequences. It should be appreciated that CP-Cas9 sequences may be based on Cas9 sequences other than that of SEQ ID NO: 28 and any examples provided herein are not meant to be limiting. Exemplary CP-Cas9 sequences are as follows:

| CP name | Sequence | SEQ ID NO: |
|---|---|---|
| CP1012 | DYKVYDVRKMIAKSEQEIGKATAKYFFYSNIMNFFKTEITLANGEIRKRPLIETN | SEQ ID NO: 87 |
| | GETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKE | |
| | VLDATLIHQSITGLYETRIDLSQLGGDGGSGGSGGSGGSGGSGGSGGDKKYSIGL | |
| | AIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIF | |
| | GNIVDEVAYHEKYPTIYHLRKKLVDSTDKADLRLIYLALAHMIKFRGHFLIEGDL | |
| | NPDNSDVDKLFIQLVQTYNQLFEENPINASGVDAKAILSARLSKSRRLENLIAQL | |
| | PGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALV | |
| | RQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMINEDKNLPN | |
| | EKVLPKHSLLYEYFTVYNELTKVKYVTEGMRKPAFLSGEQKKAIVDLLFKTNRKV | |
| | TVKQLKEDYFKKIECFDSVEISGVEDRENASLGTYHDLLKIIKDKDFLDNEENED | |
| | ILEDIVLILTLFEDREMIEERLKTYAHLEDDKVMKQLKRRRYTGWGRLSRKLING | |
| | IRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSRE | |
| | RMKRIEEGIKELGSQILKEHPVENTQLQNEKLYLYYLQNGRDMYVDQELDINRLS | |
| | DYDVDHIVPQSFLKDDSIDNKVLTRSDKNRGKSDNVPSEEVVKKMKNYWRQLLNA | |
| | KLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIK | |
| | KYPKLESEFVYG | |
| CP1028 | EIGKATAKYFFYSNIMNFFKTEITLANGEIRKRPLIETNGETGEIVWDKGRDFAT | SEQ ID NO: 88 |
| | VRKVLSMPQVNIVKKTEVQTGGFSKESILPKRNSDKLIARKKDWDPKKYGGFDSP | |
| | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGDGGSGGSGGSGGSGGSGGSGGMDKKYSIGLAIGTNSVGWAVITDE | |
| | YKVPSKKFKVLGNTDRHSIKKNLIGALLFDSGETAEATRLKRTARRRYTRRKNRI | |
| | CYLQEIFSNEMAKVDDSFFHRLEESFLVEEDKKHERHPIFGNIVDEVAYHEKYPT | |
| | IYHLRKKLVDSTDKADLRLIYLALAHMIKFRGHFLIEGDLNPDNSDVDKLFIQLV | |
| | QTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDA | |
| | ILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFT | |
| | VYNELTKVKYVTEGMRKPAFLSGEQKKAIVDLLFKTNRKVTVKQLKEDYFKKIEC | |
| | FDSVEISGVEDRFNASLGTYHDLLKIIKDKDELDNEENEDILEDIVLTLTLFEDR | |
| | EMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQ | |
| | ILKEHPVENTQLQNEKLYLYYLQNGRDMYVDQELDINRLSDYDVDHIVPQSFLKD | |
| | DSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLK | |
| | SKLVSDFRKDFQFYKVREINNYHHAHDAYLNAVVGTALIKKYPKLESEFVYGDYK | |
| | VYDVRKMIAKSEQ | |
| CP1041 | NIMNFFKTEITLANGEIRKRPLIETNGETGEIVWDKGRDFATVRKVLSMPQVNIV | SEQ ID NO: 89 |
| | KKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGDGG | |
| | SGGSGGSGGSGGSGGSGGDKKYSIGLAIGTNSVGWAVITDEYKVPSKKFKVLGNT | |
| | DRHSIKKNLIGALLFDSGETAEATRLKRTARRRYTRRKNRICYLQEIFSNEMAKV | |
| | DDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINA | |
| | SGVDAKAILSARLSKSRRLENLIAQLPGEKKNGLFGNLIALSLGLIPNFKSNFDL | |
| | AEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGAS | |
| | QEEFYKFIKPILEKMDGTEELLVKLNREDLLRKQRTEDNGSIPHQIHLGELHAIL | |
| | RRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEG | |
| | MRKPAFLSGEQKKAIVDLLFKTNRKVTVKQLKEDYFKKIECFDSVEISGVEDREN | |
| | ASLGTYHDLLKIIKDKDELDNEENEDILEDIVLTLTLFEDREMIEERLKTYAHLE | |
| | DDKVMKQLKRRRYTGWGRLSRKLINGIRDKQSGKTILDELKSDGFANRNEMQLIH | |
| | DDSLTFKEDIQKAQVSGQGDSLHEHIANLAGSPAIKKGILQTVKVVDELVKVMGR | |
| | HKPENIVIEMARENQTTQKGQKNSRERMKRIEEGIKELGSQILKEHPVENTQLQN | |
| | EKLYLYYLQNGRDMYVDQELDINRLSDYDVDHIVPQSFLKDDSIDNKVLTRSDKN | |
| | RGKSDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDFQFY | |
| | KVREINNYHHAHDAYLNAVVGTALIKKYPKLESEFVYGDYKVYDVRKMIAKSEQE | |
| | IGKATAKYFFYS | |
| CP1249 | PEDNEQKQLFVEQHKHYLDEIIEQISEFSKRVILADANLDKVLSAYNKHRDKPIR | SEQ ID NO: 90 |
| | EQAENIIHLFTLTNLGAPAAFKYFDTTIDRKRYTSTKEVLDATLIHQSITGLYET | |
| | RIDLSQLGGDGGSGGSGGSGGSGGSGGSGGMDKKYSIGLAIGTNSVGWAVITDEY | |
| | KVPSKKFKVLGNTDRHSIKKNLIGALLFDSGETAEATRLKRTARRRYTRRKNRIC | |
| | YLQEIFSNEMAKVDDSFFHRLEESELVEEDKKHERHPIFGNIVDEVAYHEKYPTI | |
| | YHLRKKLVDSTDKADLRLIYLALAHMIKFRGHFLIEGDLNPDNSDVDKLFIQLVQ | |
| | TYNQLFEENPINASGVDAKAILSARLSKSRRLENLIAQLPGEKKNGLEGNLIALS | |
| | LGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECE | |
| | DSVEISGVEDRFNASLGTYHDLLKIIKDKDELDNEENEDILEDIVLTLTLFEDRE | |
| | MIEERLKTYAHLFDDKVMKQLKRRRYTGWGRLSRKLINGIRDKQSGKTILDELKS | |
| | DGFANRNFMQLIHDDSLTFKEDIQKAQVSGQGDSLHEHIANLAGSPAIKKGILQT | |
| | VKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGDYKV | |
| | YDVRKMIAKSEQEIGKATAKYFFYSNIMNFFKTEITLANGEIRKRPLIEINGETG | |
| | EIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNE | |
| | LYLASHYEKLKGS | |
| CP1300 | KPIREQAENIIHLFTLINLGAPAAFKYFDTTIDRKRYTSTKEVLDATLIHQSITG | SEQ ID NO: 91 |
| | LYETRIDLSQLGGDGGSGGSGGSGGSGGSGGSGGDKKYSIGLAIGTNSVGWAVIT | |
| | DEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAKNLS | |
| | DAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKI | |
| | ECFDSVEISGVEDRENASLGTYHDLLKIIKDKDELDNEENEDILEDIVLTLTLFE | |
| | DREMIEERLKTYAHLFDDKVMKQLKRRRYTGWGRLSRKLINGIRDKQSGKTILDF | |
| | LKSDGFANRNEMQLIHDDSLTFKEDIQKAQVSGQGDSLHEHIANLAGSPAIKKGI | |
| | LQTVKVVDELVKVMGRHKPENIVIEMARENQTTQKGQKNSRERMKRIEEGIKELG | |
| | SQILKEHPVENTQLQNEKLYLYYLQNGRDMYVDQELDINRLSDYDVDHIVPQSFL | |
| | KDDSIDNKVLTRSDKNRGKSDNVPSEEVVKKMKNYWRQLLNAKLITQRKEDNLTK | |
| | AERGGLSELDKAGFIKRQLVETRQITKHVAQILDSRMNTKYDENDKLIREVKVIT | |
| | LKSKLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETNG | |
| | ETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANL | |
| | DKVLSAYNKHRD | |

The Cas9 circular permutants that may be useful in the base editing constructs described herein. Exemplary C-terminal fragments of Cas9, based on the Cas9 of SEQ ID NO: 28, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| CP name | Sequence | SEQ ID NO: |
|---|---|---|
| CP1012 C- | DYKVYDVRKMIAKSEQEIGKATAKYFFYSNIMNFFKTEITLANGEIRKRPLIETN | 92 |
| terminal | GETGEIVWDKGRDFATVRKVLSMPQVNIVKKTEVQTGGFSKESILPKRNSDKLIA | |
| fragment | RKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLINLGAPAAFKYFDTTIDRKRYTSTKE | |
| | VLDATLIHQSITGLYETRIDLSQLGGD | |
| CP1028 C- | EIGKATAKYFFYSNIMNFFKTEITLANGEIRKRPLIEINGETGEIVWDKGRDFAT | 93 |
| terminal | VRKVLSMPQVNIVKKTEVQTGGFSKESILPKRNSDKLIARKKDWDPKKYGGEDSP | |
| fragment | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGD | |
| CP1041 C- | NIMNFFKTEITLANGEIRKRPLIETNGETGEIVWDKGRDFATVRKVLSMPQVNIV | 94 |
| terminal | KKTEVQTGGFSKESILPKRNSDKLIARKKDWDPKKYGGFDSPTVAYSVLVVAKVE | |
| fragment | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGD | |
| CP1249 C- | PEDNEQKQLFVEQHKHYLDEIIEQISEFSKRVILADANLDKVLSAYNKHRDKPIR | 95 |
| terminal | EQAENIIHLFTLINLGAPAAFKYFDTTIDRKRYTSTKEVLDATLIHQSITGLYET | |
| fragment | RIDLSQLGGD | |
| CP1300 C- | KPIREQAENIIHLFTLTNLGAPAAFKYFDTTIDRKRYTSTKEVLDATLIHQSITG | 96 |
| terminal | LYETRIDLSQLGGD | |
| fragment | | |

Cas9 Variants with Modified PAM Specificities

The base editors of the present disclosure may also comprise Cas9 variants with modified PAM specificities. For example, the base editors described herein may utilize any naturally occurring or engineered variant of SpCas9 having expanded and/or relaxed PAM specificities which are described in the literate, including in Nishimasu et al., “Engineered CRISPR-Cas9 nuclease with expanded targeting space,” Science, 2018, 361: 1259-1262; Chatterjee et al., “Robust Genome Editing of Single-Base PAM Targets with Engineered ScCas9 Variants,” BioRxiv, Apr. 26, 2019 Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparagine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the Cas9 protein comprises a combination of mutations that exhibit activity on a target sequence comprising a 5′-NAA-3′ PAM sequence at its 3′-end. In some embodiments, the combination of mutations are present in any one of the clones listed in Table 1. In some embodiments, the combination of mutations are conservative mutations of the clones listed in Table 1. In some embodiments, the Cas9 protein comprises the combination of mutations of any one of the Cas9 clones listed in Table A.

1.

TABLE A
NAA PAM Clones
Mutations from wild-type SpCas9 (e.g., SEQ ID NO: 28)

D177N, K218R, D614N, D1135N, P1137S, E1219V, A1320V, A1323D, R1333K
D177N, K218R, D614N, D1135N, E1219V, Q1221H, H1264Y, A1320V, R1333K
A10T, I322V, $409I, E427G, G715C, D1135N, E1219V, Q1221H, H1264Y, A1320V, R1333K
A367T, K710E, R1114G, D1135N, P1137S, E1219V, Q1221H, H1264Y, A1320V, R1333K
A10T, I322V, S409I, E427G, R753G, D861N, D1135N, K1188R, E1219V, Q1221H, H1264H,
A1320V, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, V743I, R753G, E762G, D1135N, D1180G, K1211R, E1219V,
Q1221H, H1264Y, A1320V, R1333K
A10T, I322V, S409I, E427G, R753G, D1135N, D1180G, K1211R, E1219V, Q1221H, H1264Y,
S1274R, A1320V, R1333K
A10T, I322V, S409I, E427G, A589S, R753G, D1135N, E1219V, Q1221H, H1264H, A1320V,
R1333K
A10T, I322V, S409I, E427G, R753G, E757K, G865G, D1135N, E1219V, Q1221H, H1264Y,
A1320V, R1333K
A10T, I322V, S409I, E427G, R654L, R753G, E757K, D1135N, E1219V, Q1221H, H1264Y,
A1320V, R1333K
A10T, I322V, S409I, E427G, K599R, M631A, R654L, K673E, V743I, R753G, N758H, E762G,
D1135N, D1180G, E1219V, Q1221H, Q1256R, H1264Y, A1320V, A1323D, R1333K
A10T, I322V, S409I, E427G, R654L, K673E, V743I, R753G, E762G, N869S, N1054D, R1114G,
D1135N, D1180G, E1219V, Q1221H, H1264Y, A1320V, A1323D, R1333K
A10T, I322V, S409I, E427G, R654L, L727I, V743I, R753G, E762G, R859S, N946D, F1134L,
D1135N, D1180G, E1219V, Q1221H, H1264Y, N1317T, A1320V, A1323D, R1333K
A10T, I322V, S409I, E427G, R654L, K673E, V743I, R753G, E762G, N803S, N869S, Y1016D,
G1077D, R1114G, F1134L, D1135N, D1180G, E1219V, Q1221H, H1264Y, V1290G, L1318S,
A1320V, A1323D, R1333K
A10T, I322V, S409I, E427G, R654L, K673E, V743I, R753G, E762G, N803S, N869S, Y1016D,
G1077D, R1114G, F1134L, D1135N, K1151E, D1180G, E1219V, Q1221H, H1264Y, V1290G,
L1318S, A1320V, R1333K
A10T, I322V, S409I, E427G, R654L, K673E, V743I, R753G, E762G, N803S, N869S, Y1016D,
G1077D, R1114G, F1134L, D1135N, D1180G, E1219V, Q1221H, H1264Y, V1290G, L1318S,
A1320V, A1323D, R1333K
A10T, I322V, S409I, E427G, R654L, K673E, F693L, V743I, R753G, E762G, N803S, N869S,
L921P, Y1016D, G1077D, F1080S, R1114G, D1135N, D1180G, E1219V, Q1221H, H1264Y,
L1318S, A1320V, A1323D, R1333K
A10T, I322V, S409I, E427G, E630K, R654L, K673E, V743I, R753G, E762G, Q768H, N803S,
N869S, Y1016D, G1077D, R1114G, F1134L, D1135N, D1180G, E1219V, Q1221H, H1264Y,
L1318S, A1320V, R1333K
A10T, I322V, S409I, E427G, R654L, K673E, F693L, V743I, R753G, E762G, Q768H, N803S,
N869S, Y1016D, G1077D, R1114G, F1134L, D1135N, D1180G, E1219V, Q1221H, G1223S,
H1264Y, L1318S, A1320V, R1333K
A10T, I322V, S409I, E427G, R654L, K673E, F693L, V743I, R753G, E762G, N803S, N869S,
L921P, Y1016D, G1077D, F1801S, R1114G, D1135N, D1180G, E1219V, Q1221H, H1264Y,
L1318S, A1320V, A1323D, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, R654L, K673E, V743I, R753G, E762G, M673I, N803S, N869S,
G1077D, R1114G, D1135N, V1139A, D1180G, E1219V, Q1221H, A1320V, R1333K
A10T, I322V, S409I, E427G, R654L, K673E, V743I, R753G, E762G, N803S, N869S, R1114G,
D1135N, E1219V, Q1221H, A1320V, R1333K

In some embodiments, the Cas9 protein comprises an amino acid sequence that is at least 80% identical to the amino acid sequence of a Cas9 protein as provided by any one of the variants of Table 1. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table A.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 28. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 28 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 28 on the same target sequence. In some embodiments, the 3′ end of the target sequence is directly adjacent to an AAA, GAA, CAA, or TAA sequence. In some embodiments, the Cas9 protein comprises a combination of mutations that exhibit activity on a target sequence comprising a 5′-NAC-3′ PAM sequence at its 3′-end. In some embodiments, the combination of mutations are present in any one of the clones listed in Table 2. In some embodiments, the combination of mutations are conservative mutations of the clones listed in Table 2. In some embodiments, the Cas9 protein comprises the combination of mutations of any one of the Cas9 clones listed in Table B.

TABLE B
NAC PAM Clones
MUTATIONS FROM WILD-TYPE SPCAS9 (E.G., SEQ ID NO: 28)

T472I, R753G, K890E, D1332N, R1335Q, T1337N
I1057S, D1135N, P1301S, R1335Q, T1337N
T472I, R753G, D1332N, R1335Q, T1337N
D1135N, E1219V, D1332N, R1335Q, T1337N
T472I, R753G, K890E, D1332N, R1335Q, T1337N
I1057S, D1135N, P1301S, R1335Q, T1337N
T472I, R753G, D1332N, R1335Q, T1337N
T472I, R753G, Q771H, D1332N, R1335Q, T1337N
E627K, T638P, K652T, R753G, N803S, K959N, R1114G, D1135N, E1219V, D1332N, R1335Q,
T1337N
E627K, T638P, K652T, R753G, N803S, K959N, R1114G, D1135N, K1156E, E1219V, D1332N,
R1335Q, T1337N
E627K, T638P, V647I, R753G, N803S, K959N, G1030R, I1055E, R1114G, D1135N, E1219V,
D1332N, R1335Q, T1337N
E627K, E630G, T638P, V647A, G687R, N767D, N803S, K959N, R1114G, D1135N, E1219V,
D1332G, R1335Q, T1337N
E627K, T638P, R753G, N803S, K959N, R1114G, D1135N, E1219V, N1266H, D1332N, R1335Q,
T1337N
E627K, T638P, R753G, N803S, K959N, I1057T, R1114G, D1135N, E1219V, D1332N, R1335Q,
T1337N
E627K, T638P, R753G, N803S, K959N, R1114G, D1135N, E1219V, D1332N, R1335Q, T1337N
E627K, M631I, T638P, R753G, N803S, K959N, Y1036H, R1114G, D1135N, E1219V, D1251G,
D1332G, R1335Q, T1337N
E627K, T638P, R753G, N803S, V875I, K959N, Y1016C, R1114G, D1135N, E1219V, D1251G,
D1332G, R1335Q, T1337N, I1348V
K608R, E627K, T638P, V647I, R654L, R753G, N803S, T804A, K848N, V922A, K959N, R1114G,
D1135N, E1219V, D1332N, R1335Q, T1337N
K608R, E627K, T638P, V647I, R753G, N803S, V922A, K959N, K1014N, V1015A,
R1114G,
D1135N, K1156N, E1219V, N1252D, D1332N, R1335Q, T1337N
K608R, E627K, R629G, T638P, V647I, A711T, R753G, K775R, K789E, N803S, K959N, V1015A,
Y1036H, R1114G, D1135N, E1219V, N1286H, D1332N, R1335Q, T1337N
K608R, E627K, T638P, V647I, T740A, R753G, N803S, K948E, K959N, Y1016S, R1114G,
D1135N, E1219V, N1286H, D1332N, R1335Q, T1337N
K608R, E627K, T638P, V647I, T740A, N803S, K948E, K959N, Y1016S, R1114G, D1135N,
E1219V, N1286H, D1332N, R1335Q, T1337N
I670S, K608R, E627K, E630G, T638P, V647I, R653K, R753G, I795L, K797N, N803S, K866R,
K890N, K959N, Y1016C, R1114G, D1135N, E1219V, D1332N, R1335Q, T1337N
K608R, E627K, T638P, V647I, T740A, G752R, R753G, K797N, N803S, K948E, K959N, V1015A,
Y1016S, R1114G, D1135N, E1219V, N1266H, D1332N, R1335Q, T1337N
I570T, A589V, K608R, E627K, T638P, V647I, R654L, Q716R, R753G, N803S, K948E, K959N,
Y1016S, R1114G, D1135N, E1207G, E1219V, N1234D, D1332N, R1335Q, T1337N
K608R, E627K, R629G, T638P, V647I, R654L, Q740R, R753G, N803S, K959N, N990S, T995S,
V1015A, Y1036D, R1114G, D1135N, E1207G, E1219V, N1234D, N1266H, D1332N, R1335Q,
T1337N
I562F, V565D, I570T, K608R, L625S, E627K, T638P, V647I, R654I, G752R, R753G, N803S,
N808D, K959N, M1021L, R1114G, D1135N, N1177S, N1234D, D1332N, R1335Q, T1337N
I562F, I570T, K608R, E627K, T638P, V647I, R753G, E790A, N803S, K959N, V1015A, Y1036H,
R1114G, D1135N, D1180E, A1184T, E1219V, D1332N, R1335Q, T1337N
I570T, K608R, E627K, T638P, V647I, R654H, R753G, E790A, N803S, K959N, V1015A, R1114G,
D1127A, D1135N, E1219V, D1332N, R1335Q, T1337N
I570T, K608R, L625S, E627K, T638P, V647I, R654I, T703P, R753G, N803S, N808D, K959N,
M1021L, R1114G, D1135N, E1219V, D1332N, R1335Q, T1337N
I570S, K608R, E627K, E630G, T638P, V647I, R653K, R753G, I795L, N803S, K866R, K890N,
K959N, Y1016C, R1114G, D1135N, E1219V, D1332N, R1335Q, T1337N
I570T, K608R, E627K, T638P, V647I, R654H, R753G, E790A, N803S, K959N, V1016A, R1114G,
D1135N, E1219V, K1246E, D1332N, R1335Q, T1337N
K608R, E627K, T638P, V647I, R654L, K673E, R753G, E790A, N803S, K948E, K959N, R1114G,
D1127G, D1135N, D1180E, E1219V, N1286H, D1332N, R1335Q, T1337N
K608R, L625S, E627K, T638P, V647I, R654I, I670T, R753G, N803S, N808D, K959N, M1021L,
R1114G, D1135N, E1219V, N1286H, D1332N, R1335Q, T1337N
E627K, M631V, T638P, V647I, K710E, R753G, N803S, N808D, K948E, M1021L, R1114G,
D1135N, E1219V, D1332N, R1335Q, T1337N, S1338T, H1349R

In some embodiments, the Cas9 protein comprises an amino acid sequence that is at least 80% identical to the amino acid sequence of a Cas9 protein as provided by any one of the variants of Table 2. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table B.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 28. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 28 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 28 on the same target sequence. In some embodiments, the 3′ end of the target sequence is directly adjacent to an AAC, GAC, CAC, or TAC sequence.

In some embodiments, the Cas9 protein comprises a combination of mutations that exhibit activity on a target sequence comprising a 5′-NAT-3′ PAM sequence at its 3′-end. In some embodiments, the combination of mutations are present in any one of the clones listed in Table 3. In some embodiments, the combination of mutations are conservative mutations of the clones listed in Table 3. In some embodiments, the Cas9 protein comprises the combination of mutations of any one of the Cas9 clones listed in Table C.

TABLE C
NAT PAM Clones
MUTATIONS FROM WILD-TYPE SPCAS9 (E. G., SEQ ID NO: 28)

K961E, H985Y, D1135N, K1191N, E1219V, Q1221H, A1320A, P1321S, R1335L
D1135N, G1218S, E1219V, Q1221H, P1249S, P1321S, D1322G, R1335L
V743I, R753G, E790A, D1135N, G1218S, E1219V, Q1221H, A1227V, P1249S, N1286K, A1293T,
P1321S, D1322G, R1335L, T1339I
F575S, M631L, R654L, V748I, V743I, R753G, D853E, V922A, R1114G D1135N, G1218S,
E1219V, Q1221H, A1227V, P1249S, N1286K, A1293T, P1321S, D1322G, R1335L, T1339I
F575S, M631L, R654L, R664K, R753G, D853E, V922A, R1114G D1135N, D1180G, G1218S,
E1219V, Q1221H, P1249S, N1286K, P1321S, D1322G, R1335L
M631L, R654L, R753G, K797E, D853E, V922A, D1012A, R1114G D1135N, G1218S, E1219V,
Q1221H, P1249S, N1317K, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, D596Y, M631L, R654L, R664K, R753G, D853E, V922A, R1114G, Y1131C, D1135N,
D1180G, G1218S, E1219V, Q1221H, P1249S, Q1256R, P1321S, D1322G, R1335L
F575S, M631L, R654L, R664K, K710E, V750A, R753G, D853E, V922A, R1114G, Y1131C,
D1135N, D1180G, G1218S, E1219V, Q1221H, P1249S, P1321S, D1322G, R1335L
F575S, M631L, K649R, R654L, R664K, R753G, D853E, V922A, R1114G, Y1131C, D1135N,
K1156E, D1180G, G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, I1057G, R1114G, Y1131C, D1135N,
D1180G, G1218S, E1219V, Q1221H, P1249S, N1308D, P1321S, D1322G, R1335L
M631L, R654L, R753G, D853E, V922A, R1114G, Y1131C, D1135N, E1150V, D1180G, G1218S,
E1219V, Q1221H, P1249S, P1321S, D1332G, R1335L
M631L, R654L, R664K, R753G, D853E, I1057V, Y1131C, D1135N, D1180G, G1218S, E1219V,
Q1221H, P1249S, P1321S, D1332G, R1335L
M631L, R654L, R664K, R753G, I1057V, R1114G, Y1131C, D1135N, D1180G, G1218S, E1219V,
Q1221H, P1249S, P1321S, D1332G, R1335L

The above description of various napDNAbps which can be use in connection with the presently disclose base editors is not meant to be limiting in any way. The base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also may also contain various modifications that alter/enhance their PAM specifities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequences or a reference Cas9 equivalent (e.g., Cas12a/Cpf1).

In a particular embodiment, the Cas9 variant having expanded PAM capabilities is SpCas9 (H840A) VRQR, having the following amino acid sequence (with the V, R, Q, R substitutions relative to the SpCas9 (H840A) of SEQ ID NO: 97 show in bold underline. In addition, the methionine residue in SpCas9 (H840) was removed for SpCas9 (H840A) VRQR) (“SpCas9-VRQR”). This SpCas9 variant possesses an altered PAM-specificity which recognizes a PAM of 5′-NGA-3′ instead of the canonical PAM of 5′-NGG-3′:

SpCas 9-VRQR
(SEQ ID NO: 97)
DKKYSIGLDIGTNSVGWAVITDEYKVPSKKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLRKKLVDSTDKADLRLIYLALAHMIKFRGH
FLIEGDLNPDNSDVDKLFIQLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITKAPLSASMIKRYDEHHQDLT
LLKALVRQQLPEKYKEIFFDQSKNGYAGYIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEEVVDKGASAQSFIERMINFD
KNLPNEKVLPKHSLLYEYFTVYNELTKVKYVTEGMRKPAFLSGEQKKAIVDLLFKTNRKVTVKQLKEDYFKKIECFDSVEISG
VEDRFNASLGTYHDLLKIIKDKDELDNEENEDILEDIVLTLILFEDREMIEERLKTYAHLEDDKVMKQLKRRRYTGWGRLSRK
LINGIRDKQSGKTILDELKSDGFANRNEMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYLQNGRDMYVDQELDIN
RLSDYDVDAIVPQSFLKDDSIDNKVLIRSDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDERKDFQFYKVREINNYHHAHDAYLNAVVGTAL
IKKYPKLESEFVYGDYKVYDVRKMIAKSEQEIGKATAKYFFYSNIMNFFKTEITLANGEIRKRPLIEINGETGEIVWDKGRDF
ATVRKVLSMPQVNIVKKTEVQTGGFSKESILPKRNSDKLIARKKDWDPKKYGGFVSPTVAYSVLVVAKVEKGKSKKLKSVKEL
LGITIMERSSFEKNPIDFLEAKGYKEVKKDLIIKLPKYSLFELENGRKRMLASARELQKGNELALPSKYVNFLYLASHYEKLK
GSPEDNEQKQLFVEQHKHYLDEIIEQISEFSKRVILADANLDKVLSAYNKHRDKPIREQAENIIHLFTLINLGAPAAFKYEDT
TIDRKQYRSTKEVLDATLIHQSITGLYETRIDLSQLGGD

In another particular embodiment, the Cas9 variant having expanded PAM capabilities is SpCas9 (H840A) VQR, having the following amino acid sequence (with the V, Q, R substitutions relative to the SpCas9 (H840A) of SEQ ID NO: 98 show in bold underline. In addition, the methionine residue in SpCas9 (H840) was removed for SpCas9 (H840A) VRQR) (“SpCas9-VQR”). This SpCas9 variant possesses an altered PAM-specificity which recognizes a PAM of 5′-NGA-3′ instead of the canonical PAM of 5′-NGG-3′:

SpCas 9-VOR
(SEQ ID NO: 98)
DKKYSIGLDIGTNSVGWAVITDEYKVPSKKFKVLGNTDRHSIKKNLIGALLFDSGETAEATRLKRTARRRYTRRKNRICYLQE
IFSNEMAKVDDSFFHRLEESELVEEDKKHERHPIFGNIVDEVAYHEKYPTIYHLRKKLVDSTDKADLRLIYLALAHMIKFRGH
FLIEGDLNPDNSDVDKLFIQLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITKAPLSASMIKRYDEHHQDLT
LLKALVRQQLPEKYKEIFFDQSKNGYAGYIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMIRKSEETITPWNFEEVVDKGASAQSFIERMINFD
KNLPNEKVLPKHSLLYEYFTVYNELTKVKYVTEGMRKPAFLSGEQKKAIVDLLFKTNRKVTVKQLKEDYFKKIECFDSVEISG
VEDRFNASLGTYHDLLKIIKDKDELDNEENEDILEDIVLTLILFEDREMIEERLKTYAHLEDDKVMKQLKRRRYTGWGRLSRK
LINGIRDKQSGKTILDFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYLQNGRDMYVDQELDIN
RLSDYDVDAIVPQSFLKDDSIDNKVLTRSDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQFYKVREINNYHHAHDAYLNAVVGTAL
IKKYPKLESEFVYGDYKVYDVRKMIAKSEQEIGKATAKYFFYSNIMNFFKTEITLANGEIRKRPLIEINGETGEIVWDKGRDF
ATVRKVLSMPQVNIVKKTEVQTGGFSKESILPKRNSDKLIARKKDWDPKKYGGFVSPTVAYSVLVVAKVEKGKSKKLKSVKEL
LGITIMERSSFEKNPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNLGAPAAFKYFDT
TIDRKQYRSTKEVLDATLIHQSITGLYETRIDLSQLGGD

In another particular embodiment, the Cas9 variant having expanded PAM capabilities is SpCas9 (H840A) VRER, having the following amino acid sequence (with the V, R, E, R substitutions relative to the SpCas9 (H840A) of SEQ ID NO: 99 are shown in bold underline. In addition, the methionine residue in SpCas9 (H840) was removed for SpCas9 (H840A) VRER) (“SpCas9-VRER”). This SpCas9 variant possesses an altered PAM-specificity which recognizes a PAM of 5′-NGCG-3′ instead of the canonical PAM of 5′-NGG-3′:

SpCas 9-VRER
(SEQ ID NO: 99)
DKKYSIGLDIGTNSVGWAVITDEYKVPSKKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLRKKLVDSTDKADLRLIYLALAHMIKFRGH
FLIEGDLNPDNSDVDKLFIQLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITKAPLSASMIKRYDEHHQDLT
LLKALVRQQLPEKYKEIFFDQSKNGYAGYIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEEVVDKGASAQSFIERMTNFD
KNLPNEKVLPKHSLLYEYFTVYNELTKVKYVTEGMRKPAFLSGEQKKAIVDLLFKTNRKVTVKQLKEDYFKKIECFDSVEISG
VEDRFNASLGTYHDLLKIIKDKDELDNEENEDILEDIVLTLTLFEDREMIEERLKTYAHLEDDKVMKQLKRRRYTGWGRLSRK
LINGIRDKQSGKTILDFLKSDGFANRNEMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYLQNGRDMYVDQELDIN
RLSDYDVDAIVPQSFLKDDSIDNKVLTRSDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDERKDFQFYKVREINNYHHAHDAYLNAVVGTAL
IKKYPKLESEFVYGDYKVYDVRKMIAKSEQEIGKATAKYFFYSNIMNFFKTEITLANGEIRKRPLIEINGETGEIVWDKGRDF
ATVRKVLSMPQVNIVKKTEVQTGGFSKESILPKRNSDKLIARKKDWDPKKYGGFVSPTVAYSVLVVAKVEKGKSKKLKSVKEL
LGITIMERSSFEKNPIDFLEAKGYKEVKKDLIIKLPKYSLFELENGRKRMLASARELQKGNELALPSKYVNFLYLASHYEKLK
GSPEDNEQKQLFVEQHKHYLDEIIEQISEFSKRVILADANLDKVLSAYNKHRDKPIREQAENIIHLETLTNLGAPAAFKYFDT
TIDRKEYRSTKEVLDATLIHQSITGLYETRIDLSQLGGD

In yet particular embodiment, the Cas9 variant having expanded PAM capabilities is SpCas9-NG, as reported in Nishimasu et al., “Engineered CRISPR-Cas9 nuclease with expanded targeting space,” Science, 2018, 361: 1259-1262, which is incorporated herein by reference. SpCas9-NG (VRVRFRR), having the following amino acid sequence substitutions: R1335V, L1111R, D1135V, G1218R, E1219F, A1322R, and T1337R relative to the canonical SpCas9 sequence (SEQ ID NO: 28. This SpCas9 has a relaxed PAM specificity, i.e., with activity on a PAM of NGH (wherein H=A, T, or C). See Nishimasu et al., “Engineered CRISPR-Cas9 nuclease with expanded targeting space,” Science, 2018, 361: 1259-1262, which is incorporated herein by reference.

SpCas 9-NG
(SEQ ID NO: 100)
MDKKYSIGLDIGTNSVGWAVITDEYKVPSKKFKVLGNTDRHSIKKNLIGALLFDSGETAEATRLKRTARRRYTRRKNRICYLQ
EIFSNEMAKVDDSFFHRLEESFLVEEDKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELLVKLNREDLLRKQRTFDNGSIPHQIH
LGELHAILRRQEDFYPFLKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTVKQLKEDYFKKIECFDSVEIS
GVEDRFNASLGTYHDLLKIIKDKDELDNEENEDILEDIVLTLILFEDREMIEERLKTYAHLEDDKVMKQLKRRRYTGWGRLSR
KLINGIRDKQSGKTILDFLKSDGFANRNEMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDERKDFQFYKVREINNYHHAHDAYLNAVVGTA
LIKKYPKLESEFVYGDYKVYDVRKMIAKSEQEIGKATAKYFFYSNIMNFFKTEITLANGEIRKRPLIEINGETGEIVWDKGRD
FATVRKVLSMPQVNIVKKTEVQTGGFSKESIRPKRNSDKLIARKKDWDPKKYGGFVSPTVAYSVLVVAKVEKGKSKKLKSVKE
LLGITIMERSSFEKNPIDFLEAKGYKEVKKDLIIKLPKYSLFELENGRKRMLASARFLQKGNELALPSKYVNFLYLASHYEKL
KGSPEDNEQKQLFVEQHKHYLDEIIEQISEFSKRVILADANLDKVLSAYNKHRDKPIREQAENIIHLFTLINLGAPRAFKYFD
TTIDRKVYRSTKEVLDATLIHQSITGLYETRIDLSQLGGD

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Mutations may also be introduced by directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. Variant Cas9s may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Any of the references noted above which relate to Cas9 or Cas9 equivalents are hereby incorporated by reference in their entireties, if not already stated so.

### Linkers

In certain embodiments, linkers may be used to link any of the peptides or peptide domains or moieties of the invention (e.g., a mitoTALE fused to a DddA).

As defined above, the term “linker,” as used herein, refers to a chemical group or a molecule linking two molecules or moieties (e.g., a binding domain (e.g., mitoTALE) and a editing domain (e.g., DddA, or portion thereof)). In some embodiments, a linker joins a binding domain (e.g., mitoTALE) and a catalytic domain (e.g., DddA, or portion thereof). In some embodiments, a linker joins a mitoTALE and DddA. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer linkers are also contemplated.

The linker may be as simple as a covalent bond, or it may be a polymeric linker many atoms in length. In certain embodiments, the linker is a polpeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included funtionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence is greater than one amino acid residues in length. In some embodiments, the linker comprises less than six amino acid in length. In some embodiments, the linker is two amino acid residues in length. In some embodiments, the linker comprises the amino acid sequence of any one of SEQ ID NOs.: 101-117.

In certain embodiments, linkers may be used to link any of the protein or protein domains described herein (e.g., a deaminase domain and a Cas9 domain). The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond e.g., a covalent bond), an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, a linker comprises the amino acid sequence SGSETPGTSESATPES (SEQ ID NO: 101), which may also be referred to as the XTEN linker. In some embodiments, the linker is 32 amino acids in length. In some embodiments, the linker comprises the amino acid sequence (SGGS)₂-SGSETPGTSESATPES-(SGGS)₂ (SEQ ID NO: 102), which may also be referred to as (SGGS)₂—XTEN-(SGGS)₂ (SEQ ID NO: 102). In some embodiments, the linker comprises the amino acid sequence, wherein n is 0, 1, 2, 3, 4, 5, 6, 7, 8, 9, or 10. In some embodiments, a linker comprises the amino acid sequence SGGS (SEQ ID NO: 104). In some embodiments, a linker comprises (SGGS)ₙ(SEQ ID NO: 104), (GGGS)ₙ (SEQ ID NO: 105), (GGGGS)ₙ(SEQ ID NO: 106), (G)ₙ (SEQ ID NO: 107), (EAAAK)ₙ (SEQ ID NO: 108), (SGGS)ₙ-SGSETPGTSESATPES-(SGGS)ₙ(SEQ ID NO: 109), (GGS)ₙ (SEQ ID NO: 110), SGSETPGTSESATPES (SEQ ID NO: 101), or (XP). (SEQ ID NO: 111) motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, a linker comprises SGSETPGTSESATPES (SEQ ID NO: 101), and SGGS (SEQ ID NO: 104). In some embodiments, a linker comprises SGGSSGSETPGTSESATPESSGGS (SEQ ID NO: 109). In some embodiments, a linker comprises SGGSSGGSSGSETPGTSESATPESSGGSSGGS (SEQ ID NO: 112). In some embodiments, a linker comprises GGSGGSPGSPAGSPTSTEEGTSESATPESGPGTSTEPSEGSAPGSPAGSPTSTEEGTSTEPS EGSAPGTSTEPSEGSAPGTSESATPESGPGSEPATSGGSGGS (SEQ ID NO: 113). In some embodiments, the linker is 24 amino acids in length. In some embodiments, the linker comprises the amino acid sequence SGGSSGGSSGSETPGTSESATPES (SEQ ID NO: 114). In some embodiments, the linker is 40 amino acids in length. In some embodiments, the linker comprises the amino acid sequence SGGSSGGSSGSETPGTSESATPESSGGSSGGSSGGSSGGS (SEQ ID NO: 115). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGSSG GS (SEQ ID NO: 116). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAPG TSTEPSEGSAPGTSESATPESGPGSEPATS (SEQ ID NO: 117). It should be appreciated that any of the linkers provided herein may be used to link a first adenosine deaminase and a second adenosine deaminase; an adenosine deaminase (e.g., a first or a second adenosine deaminase) and a napDNAbp; a napDNAbp and an NLS; or an adenosine deaminase (e.g., a first or a second adenosine deaminase) and an NLS.

In some embodiments, any of the fusion proteins provided herein, comprise an adenosine or a cytidine deaminase and a napDNAbp that are fused to each other via a linker. In some embodiments, any of the fusion proteins provided herein, comprise a first adenosine deaminase and a second adenosine deaminase that are fused to each other via a linker. In some embodiments, any of the fusion proteins provided herein, comprise an NLS, which may be fused to an adenosine deaminase (e.g., a first and/or a second adenosine deaminase), a nucleic acid programmable DNA binding protein (napDNAbp). Various linker lengths and flexibilities between an adenosine deaminase (e.g., an engineered ecTadA) and a napDNAbp (e.g., a Cas9 domain), and/or between a first adenosine deaminase and a second adenosine deaminase can be employed (e.g., ranging from very flexible linkers of the form (GGGGS)ₙ (SEQ ID NO: 106), (GGGGS)ₙ (SEQ ID NO: 106), and (G)ₙ (SEQ ID NO: 107) to more rigid linkers of the form (EAAAK)ₙ (SEQ ID NO: 108), (SGGS)ₙ(SEQ ID NO: 104), SGSETPGTSESATPES (SEQ ID NO: 101) (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference) and (XP). (SEQ ID NO: 111)) in order to achieve the optimal length for deaminase activity for the specific application. In some embodiments, n is 1, 2, 3, 4, 5, 6, 7, 8, 9, 10, 11, 12, 13, 14, or 15. In some embodiments, the linker comprises a (GGS)ₙ(SEQ ID NO: 110) motif, wherein n is 1, 3, or 7. In some embodiments, the adenosine deaminase and the napDNAbp, and/or the first adenosine deaminase and the second adenosine deaminase of any of the fusion proteins provided herein are fused via a linker comprising the amino acid sequence SGSETPGTSESATPES (SEQ ID NO: 101), SGGS (SEQ ID NO: 104), SGGSSGSETPGTSESATPESSGGS (SEQ ID NO: 109), SGGSSGGSSGSETPGTSESATPESSGGSSGGS (SEQ ID NO: 102), or GGSGGSPGSPAGSPTSTEEGTSESATPESGPGTSTEPSEGSAPGSPAGSPTSTEEGTSTEPS EGSAPGTSTEPSEGSAPGTSESATPESGPGSEPATSGGSGGS (SEQ ID NO: 113). In some embodiments, the linker is 24 amino acids in length. In some embodiments, the linker comprises the amino acid sequence SGGSSGGSSGSETPGTSESATPES (SEQ ID NO: 114). In some embodiments, the linker is 32 amino acids in length. In some embodiments, the linker is 32 amino acids in length. In some embodiments, the linker comprises the amino acid sequence (SGGS)₂—SGSETPGTSESATPES-(SGGS)₂ (SEQ ID NO: 102), which may also be referred to as (SGGS)₂—XTEN-(SGGS)₂ (SEQ ID NO: 102). In some embodiments, the linker comprises the amino acid sequence, wherein n is 0, 1, 2, 3, 4, 5, 6, 7, 8, 9, or 10. In some embodiments, the linker is 40 amino acids in length. In some embodiments, the linker comprises the amino acid sequence SGGSSGGSSGSETPGTSESATPESSGGSSGGSSGGSSGGS (SEQ ID NO: 115). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGSSG GS (SEQ ID NO: 116). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAPG TSTEPSEGSAPGTSESATPESGPGSEPATS (SEQ ID NO: 117).

### Uracil Glycosylase Inhibitor (UGI)

In some embodiments, the fusion proteins of the disclosure comprises a UGI. When the DddA enzyme is employed and deaminates the target nucleotide, it may trigger uracil repair activity in the cell, thereby causing excision of the deaminated nucleotide. This may cause degradation of the nucleic acid or otherwise inhibit the effect of the correction or nucleotide alteration induced by the fusion protein. To inhibit this activity, a UGI may be desired. In some embodiments, the first and/or second fusion protein comprises more than one UGI. In some embodiments, the first and/or second fusion protein comprises two UGIs. In some embodiments, the first and/or second fusion protein contains two UGIs. The UGI or multiple UGIs may be appended or attached to any portion of the fusion protein. In some embodiments, the UGI is attached to the first or second portion of a DddA in the first or second fusion protein. In some embodiments, a second UGI is attached to the first UGI which is attached to the first or second portion of a DddA in the first or second fusion protein.

In other embodiments, the base editors described herein may comprise one or more uracil glycosylase inhibitors. The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 118. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 118. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 118. In some embodiments, a UGI comprises an amino acid sequence homologous to the amino acid sequence set forth in SEQ ID NO: 118, or an amino acid sequence homologous to a fragment of the amino acid sequence set forth in SEQ ID NO: 118. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 118. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 118. In some embodiments, the UGI comprises the following amino acid sequence:

### Uracil-DNA Glycosylase Inhibitor:

>sp|P14739|UNGI_BPPB2
(SEQ ID NO: 118)
MTNLSDIIEKETGKQLVIQESILMLPEEVEEVIGNKPESDILVHTAYDE
STDENVMLLTSDAPEYKPWALVIQDSNGENKIKML.

The base editors described herein may comprise more than one UGI domain, which may be separated by one or more linkers as described herein. It will also be understood that in the context of the herein disclosed base editors, the UGI domain may be linked to a deaminase domain.

In some embodiments, a UGI is absent from a base editor. In some embodiments, where a base editor comprises a ZFP or mitoZFP, UGIs are removed or are absent from the base editor. In some embodiments, the removal and/or absence of UGIs increases the activity of a DddA.

### NLS Domains

In various embodiments, the fusion proteins may comprise one or more nuclear localization sequences (NLS), which help promote translocation of a protein into the cell nucleus. Such sequences are well-known in the art and can include the following examples:

| | | SEQUENCE |
|---|---|---|
| DESCRIPTION | SEQUENCE | IDENTIFIER |
| NLS OF SV40 | PKKKRKV | 119 |
| LARGE T-AG | | |
| NLS OF | VSRKRPRP | 120 |
| POLYOMA | | |
| LARGE T-AG | | |
| NLS OF C- | PAAKRVKLD | 121 |
| MYC | | |
| NLS OF TUS- | KLKIKRPVK | 122 |
| PROTEIN | | |
| NLS OF | EGAPPAKRAR | 123 |
| HEPATITIS D | | |
| VIRUS | | |
| ANTIGEN | | |
| NLS OF | PPQPKKKPLDGE | 124 |
| MURINE P53 | | |
| NLS | MKRTADGSEFESPKKKRKV | 125 |
| NLS OF | AVKRPAATKKAGQAKKKKLD | 126 |
| NUCLEOPLAS | | |
| MIN | | |
| NLS OF PE1 | SGGSKRTADGSEFEPKKKRKV | 127 |
| AND PE2 | | |
| NLS OF EGL- | MSRRRKANPTKLSENAKKLAKEV | 128 |
| 13 | EN | |
| NLS | MDSLLMNRRKFLYQFKNVRWAKG | 129 |
| | RRETYLC | |

The NLS examples above are non-limiting. The PE fusion proteins may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

### Split-Intein Domains

It will be understood that in some embodiments (e.g., delivery of a base editor in vivo using AAV particles), it may be advantageous to split a polypeptide (e.g., a deaminase or a napDNAbp) or a fusion protein (e.g., a base editor) into an N-terminal half and a C-terminal half, delivery them separately, and then allow their colocalization to reform the complete protein (or fusion protein as the case may be) within the cell. Separate halves of a protein or a fusion protein may each comprise a split-intein tag to facilitate the reformation of the complete protein or fusion protein by the mechanism of protein trans splicing.

Protein trans-splicing, catalyzed by split inteins, provides an entirely enzymatic method for protein ligation. A split-intein is essentially a contiguous intein (e.g. a mini-intein) split into two pieces named N-intein and C-intein, respectively. The N-intein and C-intein of a split intein can associate non-covalently to form an active intein and catalyze the splicing reaction essentially in same way as a contiguous intein does. Split inteins have been found in nature and also engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

As used herein, the “N-terminal split intein (In)” refers to any intein sequence that comprises an N-terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last β-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the Ic.

In some embodiments of the invention, a peptide linked to an Ic or an In can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to an Ic can comprise one or more chemically reactive groups including, among others, ketone, aldehyde, Cys residues and Lys residues. The N-intein and C-intein of a split intein can associate non-covalently to form an active intein and catalyze the splicing reaction when an “intein-splicing polypeptide (ISP)” is present. As used herein, “intein-splicing polypeptide (ISP)” refers to the portion of the amino acid sequence of a split intein that remains when the Ic, In, or both, are removed from the split intein. In certain embodiments, the In comprises the ISP. In another embodiment, the Ic comprises the ISP. In yet another embodiment, the ISP is a separate peptide that is not covalently linked to In nor to Ic.

Split inteins may be created from contiguous inteins by engineering one or more split sites in the unstructured loop or intervening amino acid sequence between the−12 conserved beta-strands found in the structure of mini-inteins. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g. micromolar) concentrations of proteins and can be carried out under physiological conditions.

### RNA-Protein Recruitment System

In various embodiments, two separate protein domains (e.g., a Cas9 domain and a double-stranded deaminase domain) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (aka “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a base editor, as well as to recruitment additional functionalities to a base editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplarly scenario a deaminase-MS2 fusion can recruit a Cas9-MCP fusion.

A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 25).

The amino acid sequence of the MCP or MS2cp is:

(SEQ ID NO: 26)
GSASNFTQFVLVDNGGTGDVTVAPSNFANGVAEWISSNSRSQAYKVTCSV
RQSSAQNRKYTIKVEVPKVATQTVGGEELPVAGWRSYLNMELTIPIFATN
SDCELIVKAMQGLLKDGNPIPSAIAANSGIY.

### Delivery

In another aspect, the present disclosure provides for the delivery of fusion proteins in vitro and in vivo using split DddA protein formulations. The presently disclosed methods for delivering fusion proteins via various methods. For example, DddA proteins have exhibited toxic effects in vivo, and so require special solutions. One such solution is formulating the DddA, and fusion protein thereof, split into pairs that are packaged into two separate rAAV particles that, when co-delivered to a cell, reconstitute the functional DddA protein. Several other special considerations to account for the unique features of fusion protein are described, including the optimization of split sites. MitoTALE-DddA and/or mitoZF-DddA and/or Cas9-DddA fusion proteins, mRNA expressing the fusion proteins, or DNA can be packaged into lipid nanoparticles, rAAV, or lentivirus and injected, ingested, or inhaled to alter genomic DNA in vivo and ex vivo, including for the purposes of establishing animal models of human disease, testing therapeutic and scientific hypotheses in animal models of human disease, and treating disease in humans.

In another aspect, the present disclosure provides for the delivery of base editors in vitro and in vivo using various strategies, including on separate vectors using split inteins and as well as direct delivery strategies of the ribonucleoprotein complex (i.e., the base editor complexed to the gRNA and/or the second-site gRNA) using techniques such as electroporation, use of cationic lipid-mediated formulations, and induced endocytosis methods using receptor ligands fused to the ribonucleoprotein complexes. Any such methods are contemplated herein.

In some aspects, the invention provides methods comprising delivering one or more base editor-encoding polynucleotides, such as or one or more vectors as described herein encoding one or more components of the base editing system described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor fusion protein that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y→F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan Al, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the gRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the gRNA.

In various embodiments, the gRNAs and the second-site nicking guide RNAs can be expressed from an appropriate promoter, such as a human U6 (hU6) promoter, a mouse U6 (mU6) promoter, or other appropriate promoter. The gRNAs and the second-site nicking guide RNAs can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

In other aspects, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

These split intein-based methods overcome several barriers to in vivo delivery. For example, the DNA encoding base editors is larger than the rAAV packaging limit, and so requires special solutions. One such solution is formulating the editor fused to split intein pairs that are packaged into two separate rAAV particles that, when co-delivered to a cell, reconstitute the functional editor protein. Several other special considerations to account for the unique features of prime editing are described, including the optimization of second-site nicking targets and properly packaging base editors into virus vectors, including lentiviruses and rAAV.

In this aspect, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

In various embodiments, the base editors may be engineered as two half proteins (i.e., a BE N-terminal half and a BE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor fusion protein, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

In some embodiments, the split site is located in the napDNAbp domain. In other embodiments, the split site is located in the RT domain. In other embodiments, the split site is located in a linker that joins the napDNAbp domain and the RT domain.

In various embodiments, split site design requires finding sites to split and insert an N- and C-terminal intein that are both structurally permissive for purposes of packaging the two half base editor domains into two different AAV genomes. Additionally, intein residues necessary for trans splicing can be incorporated by mutating residues at the N terminus of the C terminal extein or inserting residues that will leave an intein “scar.”

In various embodiments, using SpCas9 nickase (SEQ ID NO: 29, 1368 amino acids) as an example, the split can between any two amino acids between 1 and 1368. Preferred splits, however, will be located between the central region of the protein, e.g., from amino acids 50-1250, or from 100-1200, or from 150-1150, or from 200-1100, or from 250-1050, or from 300-1000, or from 350-950, or from 400-900, or from 450-850, or from 500-800, or from 550-750, or from 600-700 of SEQ ID NO: 29. In specific exemplary embodiments, the split site may be between 740/741, or 801/802, or 1010/1011, or 1041/1042. In other embodiments the split site may be between 1/2, 2/3, 3/4, 4/5, 5/6, 6/7, 7/8, 8/9, 9/10, 10/11, 12/13, 14/15, 15/16, 17/18, 19/20 . . . 50/51 . . . 100/101 . . . 200/201 . . . 300/301 . . . 400/401 . . . 500/501 . . . 600/601 . . .

700/701 . . . 800/801 . . . 900/901 . . . 1000/1001 . . . 1100/1101 . . . 1200/1201 . . . 1300/1301 . . . and 1367/1368, including all adjacent pairs of amino acid residues.

In various embodiments, the split intein sequences can be engineered by from the following intein sequences.

2-4 INTEIN:
(SEQ ID NO: 17)
CLAEGTRIFDPVTGTTHRIEDVVDGRKPIHVVAAAKDGTLLARPVVSWFDQGTRDVIGL
RIAGGAIVWATPDHKVLTEYGWRAAGELRKGDRVAGPGGSGNSLALSLTADQMVSAL
LDAEPPILYSEYDPTSPFSEASMMGLLTNLADRELVHMINWAKRVPGFVDLTLHDQAH
LLECAWLEILMIGLVWRSMEHPGKLLFAPNLLLDRNQGKCVEGMVEIFDMLLATSSRF
RMMNLQGEEFVCLKSIILLNSGVYTFLSSTLKSLEEKDHIHRALDKITDTLIHLMAKAGL
TLQQQHQRLAQLLLILSHIRHMSNKGMEHLYSMKYKNVVPLYDLLLEMLDAHRLHAG
GSGASRVQAFADALDDKFLHDMLAEELRYSVIREVLPTRRARTFDLEVEELHTLVAEG
VVVHNC
3-2 INTEIN
(SEQ ID NO: 18)
CLAEGTRIFDPVTGTTHRIEDVVDGRKPIHVVAVAKDGTLLARPVVSWFDQGTRDVIGL
RIAGGAIVWATPDHKVLTEYGWRAAGELRKGDRVAGPGGSGNSLALSLTADQMVSAL
LDAEPPILYSEYDPTSPFSEASMMGLLTNLADRELVHMINWAKRVPGFVDLTLHDQAH
LLECAWLEILMIGLVWRSMEHPGKLLFAPNLLLDRNQGKCVEGMVEIFDMLLATSSRF
RMMNLQGEEFVCLKSIILLNSGVYTFLSSTLKSLEEKDHIHRALDKITDTLIHLMAKAGL
TLQQQHQRLAQLLLILSHIRHMSNKGMEHLYSMKYTNVVPLYDLLLEMLDAHRLHAG
GSGASRVQAFADALDDKFLHDMLAEELRYSVIREVLPTRRARTFDLEVEELHTLVAEG
VVVHNC
30R3-1 INTEIN
(SEQ ID NO: 19)
CLAEGTRIFDPVTGTTHRIEDVVDGRKPIHVVAAAKDGTLLARPVVSWFDQGTRDVIGL
RIAGGATVWATPDHKVLTEYGWRAAGELRKGDRVAGPGGSGNSLALSLTADQMVSA
LLDAEPPIPYSEYDPTSPFSEASMMGLLTNLADRELVHMINWAKRVPGFVDLTLHDQA
HLLECAWLEILMIGLVWRSMEHPGKLLFAPNLLLDRNQGKCVEGMVEIFDMLLATSSR
FRMMNLQGEEFVCLKSIILLNSGVYTFLSSTLKSLEEKDHIHRALDKITDTLIHLMAKAG
LTLQQQHQRLAQLLLILSHIRHMSNKGMEHLYSMKYKNVVPLYDLLLEMLDAHRLHA
GGSGASRVQAFADALDDKFLHDMLAEGLRYSVIREVLPTRRARTFDLEVEELHTLVAE
GVVVHNC
30R3-2 INTEIN
(SEQ ID NO: 20)
CLAEGTRIFDPVTGTTHRIEDVVDGRKPIHVVAAAKDGTLLARPVVSWFDQGTRDVIGL
RIAGGATVWATPDHKVLTEYGWRAAGELRKGDRVAGPGGSGNSLALSLTADQMVSA
LLDAEPPILYSEYDPTSPFSEASMMGLLTNLADRELVHMINWAKRVPGFVDLTLHDQA
HLLECAWLEILMIGLVWRSMEHPGKLLFAPNLLLDRNQGKCVEGMVEIFDMLLATSSR
FRMMNLQGEEFVCLKSIILLNSGVYTFLSSTLKSLEEKDHIHRALDKITDTLIHLMAKAG
LTLQQQHQRLAQLLLILSHIRHMSNKGMEHLYSMKYKNVVPLYDLLLEMLDAHRLHA
GGSGASRVQAFADALDDKFLHDMLAEELRYSVIREVLPTRRARTFDLEVEELHTLVAE
GVVVHNC
30R3-3 INTEIN
(SEQ ID NO: 21)
CLAEGTRIFDPVTGTTHRIEDVVDGRKPIHVVAAAKDGTLLARPVVSWFDQGTRDVIGL
RIAGGATVWATPDHKVLTEYGWRAAGELRKGDRVAGPGGSGNSLALSLTADQMVSA
LLDAEPPIPYSEYDPTSPFSEASMMGLLTNLADRELVHMINWAKRVPGFVDLTLHDQA
HLLECAWLEILMIGLVWRSMEHPGKLLFAPNLLLDRNQGKCVEGMVEIFDMLLATSSR
FRMMNLQGEEFVCLKSIILLNSGVYTFLSSTLKSLEEKDHIHRALDKITDTLIHLMAKAG
LTLQQQHQRLAQLLLILSHIRHMSNKGMEHLYSMKYKNVVPLYDLLLEMLDAHRLHA
GGSGASRVQAFADALDDKFLHDMLAEELRYSVIREVLPTRRARTFDLEVEELHTLVAE
GVVVHNC
37R3-1 INTEIN
((SEQ ID NO: 22)
CLAEGTRIFDPVTGTTHRIEDVVDGRKPIHVVAAAKDGTLLARPVVSWFDQGTRDVIGL
RIAGGATVWATPDHKVLTEYGWRAAGELRKGDRVAGPGGSGNSLALSLTADQMVSA
LLDAEPPILYSEYNPTSPFSEASMMGLLTNLADRELVHMINWAKRVPGFVDLTLHDQA
HLLERAWLEILMIGLVWRSMEHPGKLLFAPNLLLDRNQGKCVEGMVEIFDMLLATSSR
FRMMNLQGEEFVCLKSIILLNSGVYTFLSSTLKSLEEKDHIHRALDKITDTLIHLMAKAG
LTLQQQHQRLAQLLLILSHIRHMSNKGMEHLYSMKYKNVVPLYDLLLEMLDAHRLHA
GGSGASRVQAFADALDDKFLHDMLAEGLRYSVIREVLPTRRARTFDLEVEELHTLVAE
GVVVHNC
37R3-2 INTEIN
(SEQ ID NO: 23)
CLAEGTRIFDPVTGTTHRIEDVVDGRKPIHVVAAAKDGTLLARPVVSWFDQGTRDVIGL
RIAGGAIVWATPDHKVLTEYGWRAAGELRKGDRVAGPGGSGNSLALSLTADQMVSAL
LDAEPPILYSEYDPTSPFSEASMMGLLTNLADRELVHMINWAKRVPGFVDLTLHDQAH
LLERAWLEILMIGLVWRSMEHPGKLLFAPNLLLDRNQGKCVEGMVEIFDMLLATSSRF
RMMNLQGEEFVCLKSIILLNSGVYTFLSSTLKSLEEKDHIHRALDKITDTLIHLMAKAGL
TLQQQHQRLAQLLLILSHIRHMSNKGMEHLYSMKYKNVVPLYDLLLEMLDAHRLHAG
GSGASRVQAFADALDDKFLHDMLAEGLRYSVIREVLPTRRARTFDLEVEELHTLVAEG
VVVHNC
37R3-3 INTEIN
(SEQ ID NO: 24)
CLAEGTRIFDPVTGTTHRIEDVVDGRKPIHVVAVAKDGTLLARPVVSWFDQGTRDVIGL
RIAGGATVWATPDHKVLTEYGWRAAGELRKGDRVAGPGGSGNSLALSLTADQMVSA
LLDAEPPILYSEYDPTSPFSEASMMGLLTNLADRELVHMINWAKRVPGFVDLTLHDQA
HLLERAWLEILMIGLVWRSMEHPGKLLFAPNLLLDRNQGKCVEGMVEIFDMLLATSSR
FRMMNLQGEEFVCLKSIILLNSGVYTFLSSTLKSLEEKDHIHRALDKITDTLIHLMAKAG
LTLQQQHQRLAQLLLILSHIRHMSNKGMEHLYSMKYKNVVPLYDLLLEMLDAHRLHA
GGSGASRVQAFADALDDKFLHDMLAEELRYSVIREVLPTRRARTFDLEVEELHTLVAE
GVVVHNC

In various embodiments, the split inteins can be used to separately deliver separate portions of a complete Base editor fusion protein to a cell, which upon expression in a cell, become reconstituted as a complete Base editor fusion protein through the trans splicing.

In some embodiments, the disclosure provides a method of delivering a Base editor fusion protein to a cell, comprising: constructing a first expression vector encoding an N-terminal fragment of the Base editor fusion protein fused to a first split intein sequence; constructing a second expression vector encoding a C-terminal fragment of the Base editor fusion protein fused to a second split intein sequence; delivering the first and second expression vectors to a cell, wherein the N-terminal and C-terminal fragment are reconstituted as the Base editor fusion protein in the cell as a result of trans splicing activity causing self-excision of the first and second split intein sequences.

In other embodiments, the split site is in the napDNAbp domain.

In still other embodiments, the split site is in the adenosine deaminase domain.

In yet other embodiments, the split site is in the linker.

In other embodiments, the base editors may be delivered by ribonucleoprotein complexes.

In this aspect, the base editors may be delivered by non-viral delivery strategies involving delivery of a base editor complexed with a gRNA (i.e., a BE ribonucleoprotein complex) by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In some aspects, the invention provides methods comprising delivering one or more fusion proteins or polynucleotides encoding such fusion proteins, such as or one or more vectors as described herein encoding one or more components of the mtDNA editing system provided herein (e.g., deamination of mitochondrial DNA by a fusion protein or multiple fusion proteins) described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor (e.g., deaminating enzyme) as described herein in combination with (and optionally complexed with) a guide domain (e.g., mitoTALE) is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner: WO 91/17424 and WO 91/16024. Delivery can be to cells (e.g., in vitro or ex vivo administration) or target tissues (e.g., in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated, and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US 2003-0087817, incorporated herein by reference.

gRNAs

Some aspects of the invention relate to guide sequences (“guide RNA” or “gRNA”) that are capable of guiding a napDNAbp or a base editor comprising a napDNAbp to a target site in a DNA molecule. In various embodiments base editors (e.g., base editors provided herein) can be complexed, bound, or otherwise associated with (e.g., via any type of covalent or non-covalent bond) one or more guide sequences, i.e., the sequence which becomes associated or bound to the base editor and directs its localization to a specific target sequence having complementarity to the guide sequence or a portion thereof. The particular design aspects of a guide sequence will depend upon the nucleotide sequence of a genomic target site of interest and the type of napDNA/RNAbp (e.g., type of Cas protein) present in the base editor, among other factors, such as PAM sequence locations, percent G/C content in the target sequence, the degree of microhomology regions, secondary structures, etc.

In embodiments relating mtDNA base editors comprising Cas9/gRNA complexes, the Cas9 and gRNA components will need to be localized to the mitochondria. Cas9 can be modified with one or more MTS as discussed herein. In addition, the guide RNA may be localized to the mitochondria using known localization techniques for mRNA localization to mitochondria.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence, such as a sequence within an SMN2 gene that comprises a C840T point mutation. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 31, 32, 33, 34, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence (e.g., a HGADFN 167 or HGADFN 188 cell line), such as by transfection with vectors encoding the components of a base editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are are well known in the art and can be used with the base editors described herein.

Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are herein incorporated by reference.

### Methods of Treatment

The instant disclosure provides methods for the treatment of a subject diagnosed with a disease associated with or caused by a point mutation that can be corrected by the mtDNA editing system provided herein (e.g., deamination of mitochondrial DNA by a fusion protein or multiple fusion proteins). For example, in some embodiments, a method is provided that comprises administering to a subject having such a disease (e.g., MELAS/Leigh syndrome and Leber's hereditary optic neuropathy, other disorders associated with a point mutation as described above), an effective amount of the mtDNA editing system provided herein (e.g., deamination of mitochondrial DNA by a fusion protein or multiple fusion proteins) described herein that corrects the point mutation or introduces a point mutation comprising desired genetic change. In some embodiments, a method is provided that comprises administering to a subject having such a disease, (e.g., MELAS/Leigh syndrome and Leber's hereditary optic neuropathy, other disorders associated with a point mutation as described above), an effective amount of the mtDNA editing system provided herein (e.g., deamination of mitochondrial DNA by a fusion protein or multiple fusion proteins) described herein that corrects the point mutation or introduces a deactivating mutation into a disease-associated gene. In some embodiments, the disease is a proliferative disease. In some embodiments, the disease is a genetic disease. In some embodiments, the disease is a mitochondrial disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides methods for the treatment of additional diseases or disorders (e.g., diseases or disorders that are associated with or caused by a point mutation that can be corrected by the mtDNA editing system provided herein (e.g., deamination of mitochondrial DNA by a fusion protein or multiple fusion proteins) provided herein). Some such diseases are described herein, and additional suitable diseases that can be treated with the strategies and fusion proteins, or nucleic acids thereof, provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different (e.g., in precursors of a mature protein and the mature protein itself), and differences in sequences from species to species may affect numbering. One of skill in the art will be able to identify the respective residue in any homologous protein and in the respective encoding nucleic acid by methods well known in the art (e.g., by sequence alignment and determination of homologous residues). Exemplary suitable diseases and disorders include, without limitation: MELAS/Leigh syndrome and Leber's hereditary optic neuropathy.

The mtDNA base editors described herein may be used to treat any mitochrondrial disease or disorder. As used herein, “mitochondrial disorders” related to disorders which are due to abnormal mitochondria such as for example, a mitochondrial genetic mutation, enzyme pathways etc. Examples of disorders include and are not limited to: loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays and susceptibility to infection.

The mitochondrial abnormalities give rise to “mitochondrial diseases” which include, but not limited to: AD: Alzheimer's Disease; ADPD: Alzheimer's Disease and Parkinsons's Disease; AMDF: Ataxia, Myoclonus and Deafness CIPO: Chronic Intestinal Pseudoobstruction with myopathy and Opthalmoplegia; CPEO: Chronic Progressive External Opthalmoplegia; DEAF: Maternally inherited DEAFness or aminoglycoside-induced DEAFness; DEMCHO: Dementia and Chorea; DMDF: Diabetes Mellitus & DeaFness; Exercise Intolerance; ESOC: Epilepsy, Strokes, Optic atrophy, & Cognitive decline; FBSN: Familial Bilateral Striatal Necrosis; FICP: Fatal Infantile Cardiomyopathy Plus, a MELAS-associated cardiomyopathy; GER: Gastrointestinal Reflux; KSS Kearns Sayre Syndrome LDYT: Leber's hereditary optic neuropathy and DYsTonia; LHON: Leber Hereditary Optic Neuropathy; LFMM: Lethal Infantile Mitochondrial Myopathy; MDM: Myopathy and Diabetes Mellitus; MELAS:

Mitochondrial Encephalomyopathy, Lactic Acidosis, and Stroke-like episodes; MEPR: Myoclonic Epilepsy and Psychomotor Regression; MERME: MERRF/MELAS overlap disease; MERRF: Myoclonic Epilepsy and Ragged Red Muscle Fibers; MHCM: Maternally Inherited Hypertrophic CardioMyopathy; MICM: Maternally Inherited Cardiomyopathy; MILS: Maternally Inherited Leigh Syndrome; Mitochondrial Encephalocardiomyopathy; Mitochondrial Encephalomyopathy; MM: Mitochondrial Myopathy; MMC: Maternal Myopathy and Cardiomyopathy; Multisystem Mitochondrial Disorder (myopathy, encephalopathy, blindness, hearing loss, peripheral neuropathy); NARP: Neurogenic muscle weakness, Ataxia, and Retinitis Pigmentosa; alternate phenotype at this locus is reported as Leigh Disease; NIDDM: Non-Insulin Dependent Diabetes Mellitus; PEM: Progressive Encephalopathy; PME: Progressive Myoclonus Epilepsy; RTT: Rett Syndrome; SIDS: Sudden Infant Death Syndrome.

In embodiments, a mitochondrial disorder that may be treatable using the mtDNA base editors described herein include Myoclonic Epilepsy with Ragged Red Fibers (MERRF); Mitochondrial Myopathy, Encephalopathy, Lactacidosis, and Stroke (MELAS); Maternally Inherited Diabetes and Deafness (MIDD); Leber's Hereditary Optic Neuropathy (LHON); chronic progressive external ophthalmoplegia (CPEO); Leigh Disease; Kearns-Sayre Syndrome (KSS); Friedreich's Ataxia (FRDA); Co-Enzyme QIO (CoQIO) Deficiency; Complex I Deficiency; Complex II Deficiency; Complex III Deficiency; Complex IV Deficiency; Complex V Deficiency; other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's Disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; and cancer.

### Pharmaceutical Compositions

Other aspects of the present disclosure relate to pharmaceutical compositions comprising any of the various components of the mtDNA editing system provided herein (e.g., deamination of mitochondrial DNA by a fusion protein or multiple fusion proteins) described herein (e.g., including, but not limited to, the mitoTALE, DddA, or portions thereof, and fusion proteins (e.g., comprising mitoTALE and portion of DddA)).

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject (e.g., for nucleic acid editing). Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition is formulated in accordance with routine procedures as a composition adapted for intravenous or subcutaneous administration to a subject (e.g., a human). In some embodiments, pharmaceutical composition for administration by injection are solutions in sterile isotonic aqueous buffer. Where necessary, the pharmaceutical can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid (e.g., sterile saline, lactated Ringer's or Hank's solution). In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising: (a) a container containing a compound of the invention in lyophilized form; and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use, or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

In another aspect, the present disclosure provides for the delivery of mtDNA base editors in vitro and in vivo using various strategies, including on separate vectors using split inteins and as well as direct delivery strategies of the ribonucleoprotein complex (i.e., the base editor complexed to the gRNA and/or the second-site gRNA) using techniques such as electroporation, use of cationic lipid-mediated formulations, and induced endocytosis methods using receptor ligands fused to the ribonucleoprotein complexes. In addition, mRNA delivery methods may also be employed. Any such methods are contemplated herein. The mtDNA BE fusion proteins, or components thereof, preferably be modified with an MTS or other signal sequence that facilitates entry of the polypeptides and the guide RNAs (in the case where a pDNAbp is Cas9) into the mitochondria.

In some aspects, the invention provides methods comprising delivering one or more base editor-encoding and/or gRNA-encoding polynucleotides, such as or one or more vectors as described herein encoding one or more components described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor fusion protein that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y→F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan Al, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the gRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the gRNA.

In various embodiments, the gRNAs can be expressed from an appropriate promoter, such as a human U6 (hU6) promoter, a mouse U6 (mU6) promoter, or other appropriate promoter. The gRNAs (if multiple) can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

In other aspects, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

These split intein-based methods overcome several barriers to in vivo delivery. For example, the DNA encoding base editors is larger than the rAAV packaging limit, and so requires special solutions. One such solution is formulating the editor fused to split intein pairs that are packaged into two separate rAAV particles that, when co-delivered to a cell, reconstitute the functional editor protein.

In this aspect, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

In various embodiments, the base editors may be engineered as two half proteins (i.e., a ABE N-terminal half and a CBE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor fusion protein, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

In some embodiments, the split site is located in the pDNAbp domain. In other embodiments, the split site is located in the double stranded deaminase domain (DddA). In other embodiments, the split site is located in a linker that joins the napDNAbp domain and the deaminase domain. Preferably, the DddA is split so as to inactive the deaminase activity until the split fragments are co-localized in the mitochondria at the target site.

In various embodiments, split site design requires finding sites to split and insert an N- and C-terminal intein that are both structurally permissive for purposes of packaging the two half base editor domains into two different AAV genomes. Additionally, intein residues necessary for trans splicing can be incorporated by mutating residues at the N terminus of the C terminal extein or inserting residues that will leave an intein “scar.”

In various embodiments, using SpCas9 nickase as an example, the split can be between any two amino acids between 1 and 1368 of SEQ ID NO: 28. Preferred splits, however, will be located between the central region of the protein, e.g., from amino acids 50-1250, or from 100-1200, or from 150-1150, or from 200-1100, or from 250-1050, or from 300-1000, or from 350-950, or from 400-900, or from 450-850, or from 500-800, or from 550-750, or from 600-700 of SEQ ID NO: 28. In specific exemplary embodiments, the split site may be between 740/741, or 801/802, or 1010/1011, or 1041/1042. In other embodiments the split site may be between 1/2, 2/3, 3/4, 4/5, 5/6, 6/7, 7/8, 8/9, 9/10, 10/11, 12/13, 14/15, 15/16, 17/18, 19/20 . . . 50/51 . . . 100/101 . . . 200/201 . . . 300/301 . . . 400/401 . . . 500/501 . . . 600/601 . . .

700/701 . . . 800/801 . . . 900/901 . . . 1000/1001 . . . 1100/1101 . . . 1200/1201 . . . 1300/1301 . . . and 1367/1368, including all adjacent pairs of amino acid residues.

In various embodiments, the split inteins can be used to separately deliver separate portions of a complete Base editor fusion protein to a cell, which upon expression in a cell, become reconstituted as a complete Base editor fusion protein through the trans splicing.

In some embodiments, the disclosure provides a method of delivering a Base editor fusion protein to a cell, comprising: constructing a first expression vector encoding an N-terminal fragment of the Base editor fusion protein fused to a first split intein sequence; constructing a second expression vector encoding a C-terminal fragment of the Base editor fusion protein fused to a second split intein sequence; delivering the first and second expression vectors to a cell, wherein the N-terminal and C-terminal fragment are reconstituted as the Base editor fusion protein in the cell as a result of trans splicing activity causing self-excision of the first and second split intein sequences.

In other embodiments, the split site is in the napDNAbp domain.

In still other embodiments, the split site is in the deaminase domain.

In yet other embodiments, the split site is in the linker.

In other embodiments, the base editors may be delivered by ribonucleoprotein complexes.

In this aspect, the base editors may be delivered by non-viral delivery strategies involving delivery of a base editor complexed with a gRNA (i.e., a ABE ribonucleoprotein complex) by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Kits, Vectors, Cells

Some aspects of this disclosure provide kits comprising a fusion protein or a nucleic acid construct comprising a nucleotide sequence encoding the various components (e.g., fusion protein) of the mtDNA editing system provided herein (e.g., deamination of mitochondrial DNA by a fusion protein or multiple fusion proteins) described herein (e.g., including, but not limited to, the mitoTALE-DddA fusion proteins, vectors or cells comprising the same). In some embodiments, the nucleotide sequence comprises a heterologous promoter that drives expression of the fusion protein editing system components described herein.

Some aspects of this disclosure provide kits comprising one or more fusion proteins or nucleic acid constructs encoding the various components of the mtDNA editing system provided herein (e.g., deamination of mitochondrial DNA by a fusion protein or multiple fusion proteins) described herein, e.g., the comprising a nucleotide sequence encoding the components of the mtDNA editing system provided herein (e.g., deamination of mitochondrial DNA by a fusion protein or multiple fusion proteins) capable of modifying a target DNA sequence. In some embodiments, the nucleotide sequence comprises a heterologous promoter that drives expression of the mtDNA editing system provided herein (e.g., deamination of mitochondrial DNA by a fusion protein or multiple fusion proteins) components.

In some embodiments, a kit further comprises a set of instructions for using the fusion proteins and/or carrying out the methods herein.

Some aspects of this disclosure provides kits comprising a nucleic acid construct, comprising (a) a nucleotide sequence encoding a fusion protein (e.g., a mitoTALE and portion of a DddA) and (b) a heterologous promoter that drives expression of the sequence of (a).

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a fusion protein system as described herein (such as by transient transfection of one or more vectors, or transfection with RNA), and modified through the activity of a fusion protein complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### OTHER EMBODIMENTS

In yet further aspects, the present application provides the following embodiments as reflected in the following numbered paragraphs:
1. A non-naturally occurring polypeptide variant comprising a double-stranded DNA deaminase activity.
2. The non-naturally occurring polypeptide variant of paragraph 1, wherein the polypeptide comprises a minimum domain conferring double-stranded DNA deaminase activity.
3. The non-naturally occurring polypeptide variant of paragraph 2, wherein the minimum domain corresponds to amino acid residues 1261-1427 of (i) full-length DddA of SEQ ID NO: 1 of Burkholderia cenocepacias, (ii) a corresponding region of any one of the DddA homologs of SEQ ID NO: 3, 5, 7, 9, 11, 13, 15, or 17, or (iii) a corresponding region of an amino acid sequence having at least 90% sequence identity to SEQ ID NO: 1.
4. The non-naturally occurring polypeptide variant of paragraph 2, wherein the minimum domain corresponds to amino acid residues 1290-1425 of (i) full-length DddA of SEQ ID NO: 1 of Burkholderia cenocepacias, (ii) a corresponding region of any one of the DddA homologs of SEQ ID NO: 3, 5, 7, 9, 11, 13, 15, or 17, or (iii) a corresponding region of an amino acid sequence having at least 90% sequence identity to SEQ ID NO: 1.
5. The non-naturally occurring polypeptide variant of paragraph 1, wherein the minimum domain corresponds to SEQ ID NO: 19, or to an amino acid sequence having at least 90% sequence identity to SEQ ID NO: 19, wherein SEQ ID NO: 19 corresponds to amino acids 1251-1427 of SEQ ID NO: 1.
6. The non-naturally occurring polypeptide variant of paragraph 1, wherein the minimum domain corresponds to SEQ ID NO: 20, or to an amino acid sequence having at least 90% sequence identity to SEQ ID NO: 20, wherein SEQ ID NO: 20 corresponds to amino acids 1290-1425 of SEQ ID NO: 1.
7. A non-naturally occurring polypeptide fragment of a double-stranded DNA deaminase.
8. The non-naturally occurring polypeptide fragment of paragraph 7, wherein the polypeptide fragment comprises a minimum domain conferring double-stranded DNA deaminase activity.
9. The non-naturally occurring polypeptide fragment of paragraph 8, wherein the minimum domain corresponds to amino acid residues 1261-1427 of (i) full-length DddA of SEQ ID NO: 1 of Burkholderia cenocepacias, (ii) a corresponding region of any one of the DddA homologs of SEQ ID NO: 3, 5, 7, 9, 11, 13, 15, or 17, or (iii) a corresponding region of an amino acid sequence having at least 90% sequence identity to SEQ ID NO: 1.
10. The non-naturally occurring polypeptide fragment of paragraph 8, wherein the minimum domain corresponds to amino acid residues 1290-1425 of (i) full-length DddA of SEQ ID NO: 1 of Burkholderia cenocepacias, (ii) a corresponding region of any one of the DddA homologs of SEQ ID NO: 3, 5, 7, 9, 11, 13, 15, or 17, or (iii) a corresponding region of an amino acid sequence having at least 90% sequence identity to SEQ ID NO: 1.
11. The non-naturally occurring polypeptide fragment of paragraph 8 having the amino acid sequence of SEQ ID NO: 19, or an amino acid sequence having at least 90% sequence identity to SEQ ID NO: 19.
12. The non-naturally occurring polypeptide fragment of paragraph 8 having the amino acid sequence of SEQ ID NO: 20, or an amino acid sequence having at least 90% sequence identity to SEQ ID NO: 20.
13. A non-naturally occurring polypeptide fragment of a double-stranded DNA deaminase obtained by splitting the deaminase in the deaminase domain at a split site.
14. The non-naturally occurring polypeptide fragment of paragraph 13, wherein the fragment corresponds to an N-terminal half fragment, wherein said fragment comprises an N-terminal portion of a split deaminase domain.
15. The non-naturally occurring polypeptide fragment of paragraph 13, wherein the fragment corresponds to a C-terminal half fragment, wherein said fragment comprises a C-terminal portion of a split deaminase domain.
16. The non-naturally occurring polypeptide fragment of paragraph 14, wherein the deaminase activity is restored upon co-localizing the N-terminal half fragment with a C-terminal half fragment.
17. The non-naturally occurring polypeptide fragment of paragraph 15, wherein the deaminase activity is restored upon co-localizing the C-terminal half fragment with an N-terminal half fragment.
18. The non-naturally occurring polypeptide fragment of any one of paragraphs 13-17, wherein the amino acid sequence of the double-stranded DNA deaminase that is split is any one of SEQ ID NO: 1, 3, 5, 7, 9, 11, 13, 15, or 17, or an amino acid sequence having at least 90% sequence identity to any one of SEQ ID NO: 1, 3, 5, 7, 9, 11, 13, 15, or 17.
19. The non-naturally occurring polypeptide fragment of any one of paragraphs 13-18, wherein the split site is at the peptide bond immediately following residue G1322, G1333, A1343, N1357, G1371, N1387, or G1397 of the amino acid sequence of SEQ ID NO: 1, or at a corresponding split site in an amino acid sequence having at least 90% sequence identity to SEQ ID NO: 1.
20. The non-naturally occurring polypeptide fragment of paragraphs 14, wherein the N-terminal half fragment comprises an amino acid sequence of SEQ ID NOs: 349 or 351, or an amino acid sequence having at least 90% sequence identity to any of SEQ ID NOs: 349 or 351.
21. The non-naturally occurring polypeptide fragment of paragraphs 15, wherein the C-terminal half fragment comprises an amino acid sequence of SEQ ID NOs: 350 or 352, or an amino acid sequence having at least 90% sequence identity to any of SEQ ID NOs: 350 or 352.
22. A base editor comprising a heterodimer having first and second monomers, said first monomer comprising a first programmable DNA binding protein and an N-terminal or C-terminal fragment of a split double-stranded DNA deaminase, and said second monomer comprising a second programmable DNA binding protein and an N-terminal or C-terminal fragment of a split double-stranded DNA deaminase, wherein dimerization of the first and second monomers reconstitutes the double-stranded DNA deaminase activity.
23. The base editor of paragraph 22, wherein the first and/or second programmable DNA binding protein are the same.
24. The base editor of paragraph 22, wherein the first and/or second programmable DNA binding protein are different.
25. The base editor of paragraph 22, wherein the first and/or second programmable DNA binding protein is a nucleic acid programmable DNA binding protein (napDNAbp).
26. The base editor of paragraph 25, wherein the napDNAbp is a Cas9 domain.
27. The base editor of of paragraph 25, wherein the napDNAbp is a nickase.
28. The base editor of paragraph 25, wherein the napDNAbp comprises an inactivated nuclease activity.
29. The base editor of paragraph 25, wherein the napDNAbp is selected from the group consisting of: Cas9, Cas12e, Cas12d, Cas12a, Cas12b1, Cas13a, Cas12c, and Argonaute and optionally has a nickase activity.
30. The base editor of paragraph 25, wherein the napDNAbp comprises an amino acid sequence selected from the group consisting of SEQ ID NO: 28, 31, 33, 35, 36-91, 353, and 354, or an amino acid sequence having at least 90% sequence identity to an amino acid sequence selected from the group consisting of: SEQ ID NOs: 8, 31, 33, 35, 36-91, 353, and 354.
31. The base editor of paragraph 22, wherein the programmable DNA binding protein is a TALE protein.
32. The base editor of paragraph 31, wherein TALE protein comprises an amino acid sequence selected from the group consisting of SEQ ID NOs: 1-12, or an amino acid sequence having at least 90% sequence identity with an amino acid sequence selected from the group consisting of SEQ ID NOs: 1-12.
33. The base editor of paragraph 22, wherein the programmable DNA binding protein is a zinc finger protein.
34. The base editor of paragraph 33, wherein zinc finger protein is a commercially available zinc finger protein.
35. The base editor of paragraph 22, wherein the programmable DNA binding protein is a mitoTALE protein.
36. The base editor of paragraph 35, wherein mitoTALE protein comprises an amino acid sequence selected from the group consisting of: SEQ ID NO: 1-12, or an amino acid sequence having at least 90% sequence identity with an amino acid sequence selected from the group consisting of SEQ ID NO: 1-12.
37. The base editor of paragraph 22, wherein the N-terminal fragment of the split double-stranded DNA deaminase double-stranded DNA deaminase domain comprises an amino acid sequence of SEQ ID NO: 349 or 351, or an amino acid sequence having at least 90% sequence identity to any of SEQ ID NO: 349 or 351.
38. The base editor of paragraph 22, wherein the C-terminal fragment of the split double-stranded DNA deaminase double-stranded DNA deaminase domain comprises an amino acid sequence of SEQ ID NO: 350 or 352, or an amino acid sequence having at least 90% sequence identity to any of SEQ ID NO: 350 or 352.
39. The base editor of paragraph 22, wherein the C-terminal fragment and the N-terminal fragment of the split double-stranded DNA deaminase are obtained by splitting a double-stranded DNA deaminase in the deaminase domain at a split site.
40. The base editor of paragraph 39, wherein the double-stranded DNA deaminase comprises an amino acid sequence of SEQ ID NO: 1, 3, 5, 7, 9, 11, 13, 15, or 17, or an amino acid sequence having at least 90% sequence identity to any one of SEQ ID NO: 1, 3, 5, 7, 9, 11, 13, 15, or 17.
41. The base editor of paragraph 39, wherein the double-stranded DNA deaminase comprises a minimum domain conferring double-stranded DNA deaminase activity.
42. The base editor of paragraph 41, wherein the minimum domain corresponds to amino acid residues 1261-1427 of (i) full-length DddA of SEQ ID NO: 1 of Burkholderia cenocepacias, (ii) a corresponding region of any one of the DddA homologs of SEQ ID NO: 3, 5, 7, 9, 11, 13, 15, or 17, or (iii) a corresponding region of an amino acid sequence having at least 90% sequence identity to SEQ ID NO: 1.
43. The base editor of paragraph 41, wherein the minimum domain corresponds to amino acid residues 1290-1425 of (i) full-length DddA of SEQ ID NO: 1 of Burkholderia cenocepacias, (ii) a corresponding region of any one of the DddA homologs of SEQ ID NO: 3, 5, 7, 9, 11, 13, 15, or 17, or (iii) a corresponding region of an amino acid sequence having at least 90% sequence identity to SEQ ID NO: 1.
44. The base editor of paragraph 41, wherein the minimum domain comprises SEQ ID NO: 19, or an amino acid sequence having at least 90% sequence identity to SEQ ID NO: 19.
45. The base editor of paragraph 41, wherein the minimum domain comprises SEQ ID NO: 20, or an amino acid sequence having at least 90% sequence identity to SEQ ID NO: 19.
46. The base editor of paragraph 39, wherein the split site is at the peptide bond immediately following residue G1322, G1333, A1343, N1357, G1371, N1387, or G1397 of the amino acid sequence of SEQ ID NO: 1, or at a corresponding split site in an amino acid sequence having at least 90% sequence identity to SEQ ID NO: 1.
47. The base editor of paragraph 22, wherein the first monomer comprises a linker that joins the first programmable DNA binding protein with the N-terminal or C-terminal fragment of the split double-stranded DNA deaminase.
48. The base editor of paragraph 22, wherein the second monomer comprises a linker that joins the first programmable DNA binding protein with the N-terminal or C-terminal fragment of the split double-stranded DNA deaminase.
49. The base editor of paragraph 47 or 48, wherein the linker comprises an amino acid sequence selected from the group consisting of: SEQ ID NOs: 101-117, 357, 358, and 359, or an amino acid sequence having at least 90% sequence identity to SEQ ID NOs: 101-117, 357, 358, or 359.
50. The base editor of paragraph 47 or 48, wherein the linker comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.
51. The base editor of paragraphs 47 or 48, wherein the linker comprises 2 amino acids.
52. The base editor of any one of paragraphs 22-51, further comprising one or more uracil glycosylase inhibitor (UGI) domains.
53. The base editor of paragraph 52, wherein the one or more UGI domains comprise an amino acid sequence selected from the group consisting of: SEQ ID NOs: 118 and 355, or an amino acid sequence having at least 90% sequence identity to SEQ ID NOs: 118 or 355.
54. The base editor of any one of paragraphs 22-53, further comprising one or more targeting sequences.
55. The base editor of paragraph 54, wherein the one or more targeting sequences is a nuclear localization sequence (NLS).
56. The base editor of paragraph 55, wherein the NLS comprises an amino acid sequence selected from the group consisting of: SEQ ID NOs: 119-129 and 356, or an amino acid sequence having at least 90% sequence identity to SEQ ID NOs: 119-129 or 356.
57. The base editor of paragraph 54, wherein the one or more targeting sequences is a mitochondrial targeting sequence (MTS).
58. The base editor of paragraph 57, wherein the MTS comprises an amino acid sequence selected from the group consisting of: SEQ ID NOs: 13, 14, and 299, or an amino acid sequence having at least 90% sequence identity to SEQ ID NOs: 13, 14, or 299.
59. The base editor of paragraph 57, wherein the MTS is an SOD2 having an amino acid sequence comprising SEQ ID NO: 13, or an amino acid sequence having at least 90% sequence identity to SEQ ID NO: 13.
60. The base editor of paragraph 57, wherein the MTS is a COX8a having an amino acid sequence comprising SEQ ID NO: 14, or an amino acid sequence having at least 90% sequence identity to SEQ ID NO: 14.
61. The base editor of paragraph 22, wherein the first and/or second monomers have one of the following structures:
   (a) [A]-[programmable DNA binding protein]-[N-terminal or C-terminal fragment of a split double-stranded DNA deaminase]-[B]; or
   (b) [A]-[N-terminal or C-terminal fragment of a split double-stranded DNA deaminase]-[programmable DNA binding protein]-[B],
      wherein “[A]” and/or “[B]” represent optional one or more additional functional domains and wherein “]-[” is an optional linker.
62. The base editor of paragraph 61, wherein the optional one or more additional functional domains are selected from the group consisting of a uracil glycosylase inhibitor (UGI) domain and a targeting domain.
63. The base editor of paragraph 62, wherein the UGI domain comprises an amino acid sequence comprising SEQ ID NO: 335, or an amino acid sequence having at least 90% sequence identity to SEQ ID NO: 335.
64. The base editor of paragraph 62, wherein the targeting domain comprises an NLS having an amino acid sequence selected from the group consisting of: SEQ ID NOs: 119-129 and 356, or an amino acid sequence having at least 90% sequence identity to SEQ ID NOs: 119-129 or 356.
65. The base editor of paragraph 62, wherein the targeting domain comprises an MTS having an amino acid sequence comprising SEQ ID NO: 299, or an amino acid sequence having at least 90% sequence identity to SEQ ID NO: 299.
66. The base editor of paragraph 22, wherein the first monomer comprises the following structure: [SOD2]-[UGI]₁₋₂-[mitoTALE]-[DddAₜₒₓ-N of SEQ ID NO: 349 or DddAₜₒₓ-C of SEQ ID NO: 350]-[UGI]₁₋₂.
67. The base editor of paragraph 66, wherein the first monomer comprises an amino acid sequence comprising SEQ ID NO: 360, or an amino acid sequence having at least 90% sequence identity to SEQ ID NO: 360.
68. The base editor of paragraph 22, wherein the first monomer comprises the following structure: [COX8A]-[UGI]₁₋₂-[mitoTALE]-[DddAₜₒₓ-N of SEQ ID NO: 351 or DddAₜₒₓ-C of SEQ ID NO: 352]-[UGI]₁₋₂.
69. The base editor of paragraph 68, wherein the first monomer comprises an amino acid sequence of SEQ ID NO: 360, or an amino acid sequence having at least 90% sequence identity to SEQ ID NO: 360.
70. The base editor of paragraph 22, wherein the second monomer comprises the following structure: [SOD2]-[UGI]₁₋₂-[mitoTALE]-[DddAₜₒₓ-N of SEQ ID NO: 349 or DddAₜₒₓ-C of SEQ ID NO: 350]-[UGI]₁₋₂.
71. The base editor of paragraph 70, wherein the second monomer comprises an amino acid sequence of SEQ ID NO: 361, or an amino acid sequence having at least 90% sequence identity to SEQ ID NO: 361.
72. The base editor of paragraph 22, wherein the second monomer comprises the following structure: [COX8A]-[UGI]₁₋₂-[mitoTALE]-[DddAₜₒₓ-N of SEQ ID NO: 351 or DddAₜₒₓ-C of SEQ ID NO: 352]-[UGI]₁₋₂.
73. The base editor of paragraph 72, wherein the second monomer comprises an amino acid sequence of SEQ ID NO: 361, or an amino acid sequence having at least 90% sequence identity to SEQ ID NO: 361.
74. The base editor of any one of paragraphs 22-74, wherein the first and second monomers bind to first and second nucleotide sequences, respectively, on either side of a target site.
75. The base editor of paragraph 74, wherein the target site comprises a target base which becomes deaminated by the base editor.
76. The base editor of paragraph 75, wherein the target base is a C.
77. The base editor of paragraph 76, wherein the C is within a 5′-TC-3′ sequence context.
78. The base editor of paragraph 76, wherein the C is within a 5′-TCC-3′ sequence context, a 5′-CCC-3′ context, a 5′-TCA-3′ sequence context, or a 5′-TCT-3′ sequence context.
79. The base editor of paragraph 74, wherein the nucleotide sequences are each on the same strand as the target base which becomes deaminated by the base editor.
80. The base editor of paragraph 75, wherein the first and second nucleotide sequences are each on the same strand as the strand comprising the target base which becomes deaminated by the base editor.
81. The base editor of paragraph 75, wherein the first and second nucleotide sequences are each on the opposite strand as the strand comprising the target base which becomes deaminated by the base editor.
82. The base editor of paragraph 75, wherein the first and second nucleotide sequences are each on the opposite strand as the strand comprising the target base which becomes deaminated by the base editor.
83. The base editor of paragraph 75, wherein the first and second nucleotide sequences are on opposing strands.
84. The base editor of any of paragraph 74, further comprising one or more guide RNAs if the first and/or second programmable DNA binding protein is a nucleic acid programmable DNA binding protein (napDNAbp), and wherein the one or more guide RNAs directs the base edito to bind to the first or second nucleotide sequence at the target site.
85. An isolated nucleic acid encoding the first monomer of the base editor of paragraph 22.
86. An isolated nucleic acid encoding the second monomer of the base editor of paragraph 22.
87. An isolated nucleic acid encoding the non-naturally occurring polypeptide variant of any of paragraphs 1-6.
88. An isolated nucleic acid encoding the non-naturally occurring polypeptide fragment of any of paragraphs 7-12.
89. A vector comprising the isolated nucleic acid of any one of paragraphs 85-88.
90. A cell comprising a vector of paragraph 89.
91. A method of editing a target nucleotide sequence at a target site, comprising contacting a target nucleotide sequence with a base editor of paragraphs 22-84, wherein the first monomer targets a first nucleotide sequence flanking a target site, and the second monomer targets a second nucleotide sequence flanking the target site, thereby inducing deamination of a target base at the target site.
92. The method of claim 91, wherein the target base is a C.
93. The method of claim 92, wherein the C is within a 5′-TC-3′ sequence context.
94. The method of paragraph 92, wherein the C is within a 5′-TCC-3′ sequence context, a 5′-CCC-3′ context, a 5′-TCA-3′ sequence context, or a 5′-TCT-3′ sequence context.
95. The method of paragraph 91, wherein the programmable DNA binding protein of the base editor is a TALE, mitoTALE, zinc finger protein, or napDNAbp.
96. The method of paragraph 91, wherein the first and/or second programmable DNA binding protein of the base editor is a napDNAbp.
97. The method of paragraph 96, wherein the napDNAbp is a Cas9 domain.
98. The method of paragraph 96, wherein the napDNAbp is a nickase.
99. The method of paragraph 96, wherein the target nucleotide sequence is a mitochondrial sequence within a mitochondria.
100. The method of paragraph 96, wherein the napDNAbp is selected from the group consisting of: Cas9, Cas12e, Cas12d, Cas12a, Cas12b1, Cas13a, Cas12c, and Argonaute and optionally has a nickase activity.
101. The method of paragraph 96, wherein the napDNAbp comprises an amino acid sequence selected from the group consisting of SEQ ID NOs: 28, 31, 33, 35, 36-91, 353, and 354, or an amino acid sequence having at least 90% sequence identity to an amino acid sequence selected from the group consisting of: SEQ ID NOs: 28, 31, 33, 35, 36-91, 353, and 354.
102. The method of paragraph 91, wherein the first and/or second programmable DNA binding protein of the base editor is a mitoTALE.
103. The method of paragraph 102, wherein mitoTALE protein comprises an amino acid sequence selected from the group consisting of: SEQ ID NO: 1-12 or an amino acid sequence having at least 90% sequence identity with an amino acid sequence selected from the group consisting of SEQ ID NO: 1-12.
104. The method of paragraph 91, wherein the method further comprises contacting the target nucleotide sequence with guide RNAs if the programmable DNA binding protein of the base editor is a napDNAbp, wherein the guide RNAs direct the first and second monomers of the base editor to the target nucleotide sequence.
105. The method of paragraph 91, wherein the editing occurs in vivo.
106. The method of paragraph 91, wherein the editing occurs ex vivo.
107. The method of paragraph 91, wherein the target nucleotide sequence is a disease gene.
108. The method of paragraph 91, wherein the target nucleotide sequence is a disease gene in a mitochondria.
109. The method of paragraph 91, wherein the method of editing a nucleotide sequence results in the treatment of a mitochondrial disease.
110. The method of paragraph 91, wherein the mitochondrial disease is MELAS/Leigh syndrome and Leber's hereditary optic neuropathy.
111. A method of delivering a base editor of any of claims 22-84 to a cell comprising transforming a cell with one or more vectors encoding the first and second monomers of the base editor, wherein once in the cell the first and second monomers are expressed and dimerize, thereby forming a base editor in the cell.
112. The method of paragraph 111, wherein the one or more vectors are viral expression vectors.
113. The method of paragraph 111, wherein one or more vectors are adeno-associated viral vectors (AAV) of any serotype.
114. The method of paragraph 111, further comprising the step of expressing a guide RNA in the cell if the first and/or second monomer comprise a napDNAbp, and wherein the guide RNA is expressed from the same vector or a different vector.
115. A method of delivering a base editor of any of paragraphs 22-84 to a mitochondria comprising transforming a cell with one or more vectors encoding the first and second monomers of the base editor, wherein once in the cell the first and second monomers are expressed, transported to the mitochondria, and dimerize therein, thereby forming a base editor in the mitochondria, wherein the first and second programmable DNA binding proteins of the base editor are mitoTALE domains.
116. The method of paragraph 115, wherein the one or more vectors are viral expression vectors.
117. The method of paragraph 115, wherein one or more vectors are adeno-associated viral vectors (AAV) of any serotype.
118. A therapeutic kit comprising one or more nucleic acid constructs, comprising:
   (i) one or more nucleic acid sequences encoding the first and second monomers of the base editor of any of paragraphs 22-84,
   (ii) a promoter that drives expression of (i).
119. The therapeutic kit of paragraph 118, further comprising an expression construct encoding one or more guide RNAs where either the first or second monomer comprises a napDNAbp.

### Examples

### Example 1: A Bacterial Double-Stranded DNA Cytidine Deaminase Toxin Enables RNA-Free Base Editing in the Mitochondrial Genome

### Background

Bacterial toxins represent a vast reservoir of biochemical diversity that can be repurposed for biomedical applications. Many toxins function in interbacterial antagonism, yet their modes of action remain largely unknown. Here, the discovery, structure, biochemical characterization, and application of DddA, an interbacterial toxin that catalyzes the unprecedented deamination of cytidines within double-stranded DNA, is reported. All previously described cytidine deaminases, including those used in base editing, operate on single-stranded DNA and thus when used for genome editing require unwinding of double-stranded DNA by macromolecules such as CRISPR-Cas9 complexed with a guide RNA. The difficulty of delivering guide RNAs into the mitochondria has thus far precluded base editing in mitochondrial DNA (mtDNA). The ability of DddA to deaminate double-stranded DNA raises the possibility of RNA-free precision base editing, rather than simple elimination of targeted mtDNA copies following double-strand DNA breaks. Split-DddA halves were engineered that are non-toxic and inactive until brought together on target DNA by adjacently bound programmable DNA-binding proteins. Fusions of the split-DddA halves, TALE array proteins, and uracil glycosylase inhibitor resulted in RNA-free DddA-derived cytosine base editors (DdCBEs) that catalyze C·G-to-T·A conversions efficiently and with high DNA sequence specificity and product purity at targeted sites within mtDNA in human cells. DddA-mediated base editing was used to model a disease-associated mtDNA mutation in human cell lines, resulting in changes in rates of respiration and oxidative phosphorylation. CRISPR-free, DddA-mediated base editing enables precision editing of mtDNA, with important basic science and biomedical implications.

Enzymes that catalyze the deamination of cytidine and adenosine play pivotal roles in precision genome editing.^{1,2} The biochemical and functional diversity of deaminases, however, remain largely unexplored. In particular, bacterial genomes contain a wide variety of uncharacterized cryptic deaminases,³ raising the possibility that some may possess unique activities that could be exploited to enable new genome editing capabilities.

Inherited or acquired mutations in mitochondrial DNA (mtDNA) can profoundly impact cell physiology and are associated with a spectrum of human diseases, ranging from rare inborn errors of metabolism,⁴ certain cancers,⁵ age-associated neurodegeneration,⁶ and even the aging process itself.^{7,8} Tools for introducing specific modifications to mtDNA are urgently needed both for modeling diseases and for their therapeutic potential. The development of such tools, however, has been constrained in part by the challenge of transporting RNAs into mitochondria, including guide RNAs required to program CRISPR-associated proteins.⁹

Each mammalian cell contains hundreds to thousands of copies of a circular mtDNA¹⁰. Homoplasmy refers to a state in which all mtDNA molecules are identical, while heteroplasmy refers to a state in which a cell contains a mixture of wild-type and mutant mtDNA. Current approaches to engineer mtDNA rely on DNA-binding proteins such as transcription activator-like effectors nucleases (mitoTALENs)¹¹⁻¹⁷ and zinc finger nucleases (mitoZFNs)¹⁸⁻²⁰ fused to mitochondrial targeting sequences to induce double-strand breaks (DSBs). Such proteins do not rely on nucleic acid programmability (e.g., such as with Cas9 domains). Linearized mtDNA is rapidly degraded,²¹⁻²³ resulting in heteroplasmic shifts to favor uncut mtDNA genomes. As a candidate therapy however, this approach cannot be applied to homoplasmic mtDNA mutations²⁴ since destroying all mtDNA copies is presumed to be harmful.²²⁻²⁵ In addition, using DSBs to eliminate heteroplasmic mtDNA mutations, which tend to be functionally recessive,²⁶ implicitly requires the edited cell to restore its wild-type mtDNA copy number. During this transient period of mtDNA repopulation, the loss of mtDNA copies could result in cellular toxicity.

A favorable alternative to targeted destruction of DNA through DSBs is precision genome editing, a capability that has not been reported for mtDNA. The ability to precisely install or correct pathogenic mutations, rather than destroy targeted mtDNA, could accelerate our ability to model mtDNA diseases in cells and animal models, and in principle could also enable therapeutic approaches that correct pathogenic mtDNA mutations.

### A Predicted Deaminase Functions as an Interbacterial Toxin

Some predicted bacterial deaminases contain sequence hallmarks that suggest they are substrates for intercellular protein delivery systems.³ These hallmarks include domains that direct transport through the type VI secretion system (T6SS).³ The T6SS mediates antagonism between Gram-negative bacteria by catalyzing the direct transfer of antibacterial toxins into contacting cells.^{27,28} Given their sequence divergence and potential functional differences from characterized deaminases, the biochemical activity of T6SS-associated deaminases were sought. Investigations were focused on a predicted deaminase (belonging to the SCP1.201-like family),³ henceforth referred to as DddA, encoded by Burkholderia cenocepacias (B. cen) (FIG. 23A). A strain of B. cen lacking dddA and the downstream predicted immunity gene, which we named dddIA, exhibited a marked growth defect when co-cultivated with the wild-type strain (FIG. 23B and FIGS. 28A-28C). The ΔdddA ΔdddI_{A} strain did not display a growth defect in co-culture with a strain lacking activity of a T6SS (ΔicmF1) or expressing DddA bearing an amino acid substitution of a predicted deaminase catalytic residue (dddA^{E1347A}). These data establish DddA as a T6SS-delivered antibacterial toxin.

Members of the deaminase superfamily are known to catalyze deamination of single-stranded DNA (ssDNA), RNA (including mRNA and tRNA), free nucleosides, nucleotides, nucleobases, and other nucleotide derivatives³. To begin to define the substrate of DddA, which belongs to a clade of predicted deaminases lacking a characterized member³, first whether or not deaminases representing the substrate range of the superfamily are toxic if ectopically expressed in bacteria was determined. The growth of E. coli was unaffected by production of deaminases that act on ssDNA, tRNA, or free cytidine (FIG. 23C). In contrast, DddA dramatically reduced the viability of E. coli (FIG. 23C and FIG. 28C). Amino acids 1264-1427 of DddA were identified as the domain that confers toxicity, referred to henceforth as DddAₜₒₓ. These findings suggested that DddA may act on a previously undescribed substrate of deaminases.

### DddA is a Double-Stranded DNA-Specific Cytidine Deaminase

To further illuminate the substrate and mechanism of DddAₜₒₓ, we determined a 2.5-A resolution co-crystal structure of DddAₜₒₓ bound to DddI_{A} (Table 2). DddAₜₒₓ adopts a typical deaminase fold consisting of a five-stranded β-sheet with buttressing helices that contribute critical catalytic residues to the active site (FIG. 23D). DddIA, the immunity protein of DddA, contains a central antiparallel β-sheet that directly occludes the active site of DddAₜₒₓ, explaining the inhibitory activity of DddIA (FIG. 23D). Structure-based homology searches revealed APOBEC family enzymes as the closest structural relatives of DddAₜₒₓ, with notable divergence at the C-terminal β-strands of the two enzymes; these strands are antiparallel with an extended intervening loop in DddAₜₒₓ, versus parallel with an intervening α-helix in APOBEC enzymes (FIGS. 23D-23E).

Given the similarity of DddAₜₒₓ and APOBEC proteins, the ability of DddAₜₒₓ to catalyze the deamination of cytidine in vitro was tested. To date, all known DNA cytidine deaminases operate on ssDNA, often with a preference for the base immediately 5′ of the substrate cytidine.²⁹ Therefore, the activity of DddAₜₒₓ on a ssDNA substrate containing cytidine was measured in all four possible 5′-NC contexts. While the activity of APOBEC3A was readily detected, DddAₜₒₓ did not catalyze uracil formation within ssDNA sequences (FIG. 23F). As a control, a related double-stranded DNA (dsDNA) substrate was included. Consistent with prior studies,³⁰ APOBEC3A did not display measurable activity against dsDNA. Unexpectedly, however, DddAₜₒₓ efficiently converted cytidine to uracil within dsDNA (FIG. 23G). A mutant DddAₜₒₓ in which we inactivated a predicted catalytic residue E1347A showed no uracil formation, indicating that deamination was dependent on DddAₜₒₓ activity. Substrates of ssRNA or dsRNA were not detectably deaminated by DddAₜₒₓ(FIGS. 29A-29B). These results collectively establish DddAₜₒₓ as an unusual cytidine deaminase that operates on dsDNA but rejects ssDNA and RNA. The name DddAₜₒₓ was derived based on these findings (double-stranded DNA deaminase toxin A). In light of these findings, we reexamined the DddAₜₒₓ structure to gain insights into the unprecedented ability of the deaminase to act on dsDNA. Superimposition of DddAₜₒₓ with an APOBEC3A-ssDNA complex suggests that cytidine is positioned similarly by the two enzymes, likely necessitating extrusion of the target cytidine from dsDNA (FIGS. 30A-30B), as observed in dsRNA-specific adenosine deaminases.³¹

If DddAₜₒₓ converts cytidine to uracil specifically within dsDNA, it was reasoned that the enzyme should be mutagenic in a manner that is dependent on uracil DNA glycosylase (UDG), a protein that initiates base excision repair (BER) through uracil removal.^{32,33} Indeed, expression of sub-lethal levels of DddAₜₒₓ in E. coli substantially increased mutation frequency, and these mutagenic effects of DddAₜₒₓ were enhanced >100-fold in an E. coli strain lacking UDG (FIG. 23H). An exploitation of the high mutational frequency incurred by sub-lethal DddAₜₒₓ levels to profile the potential sequence context specificity of the enzyme was sought. Whole-genome sequencing was performed on five E. coli lineages that experienced serial DddAₜₒₓ exposure and clonal bottlenecking, and five control strains that underwent a similar regimen in the presence of inactivated DddAₜₒₓ (E1347A). Consistent with the mutation frequency measurements, ˜50-fold more total SNPs were observed in strains exposed to active DddAₜₒₓ(997) than strains producing the inactive enzyme (17), and >99% of the DddAₜₒₓ-dependent SNPs were C·G-to-T·A transitions (FIGS. 31A-31C). Alignment of sequences flanking the converted cytidine within these C·G-to-T·A mutations revealed a strong preference for 5′-TC contexts (FIG. 23I), which matches the substrate sequence preference of the enzyme in vitro (FIG. 31D). Together, these findings reveal that DddAₜₒₓ deaminates dsDNA substrates in vitro and in bacterial cells with a preference for 5′-TC contexts, likely through cytosine extrusion.

Identifying Split DddAₜₒₓ Halves that are Non-Toxic and Catalytically Competent

Current base editors deaminate nucleotides in single-stranded DNA loops created by RNA-guided CRISPR proteins.^{2,34,35} The ability of DddAₜₒₓ to deaminate cytidines in dsDNA raises the possibility of using RNA-free programmable dsDNA-binding proteins such as zinc-finger arrays³⁶ or TALE arrays³⁷ to direct DddAₜₒₓ to target cytidines in dsDNA. The resulting dsDNA cytosine base editor would enable base editing without requiring guide RNAs, raising the possibility of base editing in systems for which RNA delivery is prohibitive, such as the mitochondria.⁹

Consistent with experiments in E. coli (FIGS. 23B-23C), expression of DddAₜₒₓ fused to programmable DNA-binding proteins was toxic to human HEK293T cells (FIGS. 40A-40C and the Supplementary Discussion section of Example 1). It was hypothesized that this toxicity could be avoided by splitting the protein into two inactive halves, one containing the N-terminus of DddAₜₒₓ(DddAₜₒₓ-N), and the other containing the C-terminus (DddAₜₒₓ-C). Ideally, these halves would reconstitute deamination activity only when assembled adjacently on target DNA, analogous to the reassembly of FokI monomers to confer nuclease activity in ZFNs³⁸ and TALENs³⁷.

Based on the crystal structure of apo-DddAₜₒₓ, seven split sites within the loop regions of DddAₜₒₓ were tested (FIG. 24A). Split variants were named to reflect the last residue of the DddAₜₒₓ-N half. Each DddAₜₒₓ half was fused to the N-terminus of dSpCas9^{39,40} or to an orthogonal engineered S. aureus SaKKH Cas9 variant (SaKKH-Cas9)⁴¹ (FIG. 24B). To enhance editing efficiencies, SaKKH-Cas9(D10A) nickase^{41,42} was used to nick the non-edited strand, thereby promoting resynthesis of the non-edited strand using the edited strand as a template, a strategy used during the development of base editors.^{34,35,43,44} Each split was assayed in its two possible fusion orientations: SaKKH-Cas9(D10A) fused to DddAₜₒₓ-N(referred to as the aureus-N orientation) or SaKKH-Cas9(D10A) fused to DddAₜₒₓ-C(referred to as the aureus-C orientation) (FIG. 24B). Dual binding of orthogonal DddAₜₒₓ-dSpCas9 half and DddAₜₒₓ-SaKKH-Cas9(D10A) half to adjacent protospacers would in principle reconstitute functional DddAₜₒₓ at target sites for cytidine deamination (FIG. 24C). The choice of protospacers was also varied to test split-DddAₜₒₓ reassembly at target site spacing distances ranging from 12 to 60 bp in intervals of ˜5-10 bp (FIGS. 41A-41C).

HEK293T cells were transfected with four plasmids: two plasmids encode DddAₜₒₓ-N or DddAₜₒₓ-C fused to either dSpCas9 or SaKKH-Cas9(D10A), and the remaining two plasmids encode two guide RNAs that direct the fusion proteins to flank a target TC (FIG. 24B). Genomic DNA was harvested three days post-transfection and analyzed by high-throughput DNA sequencing.

Among active split-DddAₜₒₓ fusions, predominantly C·G-to-T·A conversions were observed in the intervening DNA sequence between the two protospacers. Editing efficiencies varied from 1.1% to 49% depending on the split site, split orientation and dsDNA spacing length (FIG. 24B and FIGS. 32A-32H). All editing efficiencies in this study report the fraction of sequenced alleles with the desired C·G-to-T·A edit among all treated cells with no enrichment or sorting. Importantly, it was observed that no on-target editing in the absence of guide RNAs (FIGS. 42A-42B) or when only one DddAₜₒₓ-Cas9 half and its guide RNA were present (FIG. 42B), indicating that editing is strictly dependent on the reassembly of both DddAₜₒₓ halves at the Cas9-specified target site.

Out of the seven split sites tested, G1333 and G1397 yielded the highest editing efficiencies (FIGS. 32A-32H and the Supplementary Discussion section in Example 1), ranging from 22-48% maximal C·G-to-T·A conversion (FIGS. 32B, 32G, and 32H). For a given fusion orientation, the editing efficiencies of target bases were dependent on their positions within the spacing region; editing at a target TC₁₁A within a 12-bp spacing region was 0.43±0.12% for G1397 aureus-N and 17±0.91% at a 17-bp spacing (FIG. 24D). Similarly, G1397 aureus-C yielded 20-22% editing efficiency at a target TC₁₄T within 17- and 23-bp spacing regions and 41±2.1% within a 44-bp spacing region (FIG. 24D). These results collectively suggest that splitting DddAₜₒₓ at G1333 and G1397 produce halves that can reconstitute a catalytically active deaminase capable of mediating efficient C·G-to-T·A conversion in human cells with appropriate spacing (˜12-23 bp, or ˜44-60 bp) between dual protospacers. Spacing length, target cytidine location within the dsDNA spacing region, and split orientation are all determinants of split DddAₜₒₓ base editing efficiency.

### TALE-Split-DddAₜₒₓ Fusions Enable RNA-Free Base Editing

Despite the potential of mitochondrial DNA editing to illuminate mitochondrial biology and treat diseases that arise from mtDNA mutations, editing mtDNA has been hampered by the lack of an effective way to import RNA into the organelle⁹. This constraint has precluded the use of RNA-guided precision editing strategies such as base editing^{34,35}, as well as other CRISPR methods⁴³. We speculated that we could use RNA-independent programmable DNA-binding proteins fused to split-DddAₜₒₓ halves to enable RNA-free base editing of mtDNA. TALE proteins contain an array of highly conserved 33- or 34-amino acid repeats that each recognize a dsDNA nucleotide. Each repeat can be programmed to target A, C, G/A, or T nucleotides^{45,46}. Arrays of TALE repeats recognize consecutive nucleotides of sufficient total length to specify a single dsDNA target sequence within a mammalian genome⁴⁷⁻⁴⁹.

While deaminases have been previously fused to zinc-finger arrays and TALE arrays, the very low activity of previously described deaminases on dsDNA results in very low editing efficiencies (<2.5% in human cells) that are spread over >150 bp around the DNA-binding site⁵⁰. Since our Cas9 fusion results indicate that DddAₜₒₓ split at sites G1333 and G1397 deaminate target TC bases in dsDNA efficiently and in a manner that limits their activity to a modest spacing region (FIG. 24D and FIGS. 32A-32H), it was speculated that fusing halves from these two DddAₜₒₓ splits to TALE array proteins programmed to bind neighboring DNA sites might result in selective and efficient RNA-free base editing in human cells. We limited DNA spacing lengths between the neighboring protospacers to 15-25 bp to minimize deamination of nearby bystander cytidines within the spacing region, and tested both split orientations to compare base editing efficiency and substrate selectivity.

Each TALE array was designed to target neighboring 17-bp sites within CCR5 in U20S cells and contained a bipartite NLS (bpNLS)^{51,52} (FIG. 33A). Among the G1333 and G1397 splits tested, only the G1333 split with DddAₜₒₓ-N fused to the upstream (left-side) TALE (Left-G 1333-DddAₜₒₓ-N+Right-G1333-DddAₜₒₓ-C) resulted in modest 3.6±0.70% editing efficiency at C₉ (the ninth nucleotide of the DNA spacing region between the two TALE array target sites) with 20±0.87% indels across 2- or 16-amino acid linkers (FIG. 33A).

We hypothesized that fusing uracil DNA glycosylase inhibitor (UGI) from B. subtilis bacteriophage PBS1 to the N- or C-terminus of each TALE-DddAₜₒₓ fusion could suppress UDG-mediated nuclear base excision repair, a strategy we previously used to develop cytosine base editors^{34,53,54}. Indeed, appending two copies of UGI (2×-UGI) to the N-terminus increased editing efficiency at C₉ by ˜8-fold to 22-27%, and reduced indels to <2.3±0.31% (FIG. 33B). In contrast, fusing 2×-UGI to the C-terminus through a 2- or 16-amino acid linker resulting in lower editing efficiency of 12±3.5% or 3.3±1.3%, respectively (FIG. 33B). Tethering UGIs to the C-terminus of the DddAₜₒₓ half may sterically hinder the deaminase, thereby impairing editing efficiencies. These results collectively demonstrate that G1333 and G1397 splits of DddAₜₒₓ can be fused to TALE arrays by a 2-amino acid linker to mediate C·G-to-T·A conversions in the nucleus of human cells, and that fusing UGI to these proteins enhances editing efficiencies and reduces indel byproducts^{34,53}

### Optimizing TALE-Split DddAₜₒₓ Fusions for Mitochondrial Base Editing

Given that TALE-split DddAₜₒₓ fusions mediated efficient RNA-free base editing of nuclear DNA in human cells, we next investigated the possibility of applying this system to achieve programmable C·G-to-T·A conversion in mtDNA. We introduced previously reported mutations into the N-terminal domain (NTD)⁵⁵ of the 14459A-mitoTALE pair¹¹ to recognize wild-type ND6, a mitochondrial gene that encodes the NADH dehydrogenase 6 subunit of complex I. Each DddAₜₒₓ half from a G1333 or G1397 split was fused to the C-terminus of the mitoTALE array protein through a 2-amino acid linker to form a mitoTALE-DddAₜₒₓ pair that flanks a 15-bp mtDNA spacing region in HEK293T cells (FIG. 34A).

Among simple mitoTALE-DddAₜₒₓ fusions (FIG. 34B), we observed the highest level of mtDNA target editing (4.9±0.17%) for the G1397 split containing DddAₜₒₓ-C fused to the right-side mitoTALE (Right-DddAₜₒₓ-C+Left-DddAₜₒₓ-N) (FIG. 34C). In contrast to nuclear-localized TALE-DddAₜₒₓ(FIG. 33B), fusing one or two UGI proteins to the N-terminus of each mitoTALE-DddAₜₒₓ half did not enhance C·G-to-T·A conversion (FIG. 25A). Appending one UGI to the C-terminus, however, increased editing levels by 3- to 10-fold compared to constructs lacking UGI. Using this Right-G1397-DddAₜₒₓ-C+Left-G1397-DddAₜₒₓ-N orientation, cytidines C6, C₇, and C₁3 (all in TC contexts) were edited with 27±2.1%, 32±2.5%, and 16±1.5% efficiencies (FIG. 25A), respectively, while C₁₁ in an AC sequence context that is disfavored by DddAₜₒₓ (FIG. 23I) was edited less efficiently (4.4±0.39%). We speculate that UGI may be inhibiting the mitochondrial isoform UDG1⁵⁶, which was previously isolated from human mitochondria and shown to have uracil excision activity in vitro and in mitochondrial extracts⁵⁷⁻⁵⁹. Importantly, removing the mitochondrial targeting signal (MTS) sequences or replacing them with a bpNLS abrogated editing (FIG. 25A), suggesting that ND6 editing is dependent on mitochondrial localization of the mitoTALE-DddAₜₒₓ fusions (see the Supplementary Discussion section of Example 1).

Fluorescence microscopy revealed that while C-terminal MTS-mitoTALE-split-DddAₜₒₓ-UGI fusions clearly localized to the mitochondria in HeLa cells, N-terminal MTS-UGI-mitoTALE-split-DddAₜₒₓ fusions remained diffused throughout the cytoplasm (FIG. 25B). These findings explain the observed dependence of editing efficiency on UGI fusion position, and suggest that the close proximity between the MTS and the N-terminal UGIs may impede mitochondrial translocation of the fusion protein.

In contrast with cytosine base editors for nuclear DNA⁵³, appending a second copy of UGI to the C-terminus of MTS-mitoTALE-split-DddAₜₒₓ-UGI did not increase mtDNA editing efficiencies for any tested target, despite exhibiting similar levels of mitochondrial localization (FIGS. 25A-25B). We speculate that adding a second UGI to the C-terminus may impede reassembly of split DddAₜₒₓ into an active deaminase.

These results collectively suggested an optimized architecture for a mitoTALE-split-DddAₜₒₓ pair in which each protein consists of (in N- to C-terminus order): an MTS, a TALE array, a 2-amino acid linker, a DddAₜₒₓ half from the G1333 or G1397 split, and one UGI protein (FIG. 25C). This architecture, hereafter referred to as DddA-derived cytosine base editor (DdCBE), represents to our knowledge the first agent capable of performing targeted nucleotide conversions in mtDNA. This precision mtDNA editing capability contrasts with previously reported uses of TALE nucleases^{11-13,16}, zinc-finger nucleases¹⁸⁻²⁰ or restriction endonucleases^{22,60} that make DSBs in mtDNA, which result primarily in copy number loss or heteroplasmic shifts in mtDNA^{11-15,18,20,61}.

Given that DddAₜₒₓ can edit cytidines on either DNA strand, intermediates containing uracils on opposing DNA strands could produce DSBs following base excision repair, thereby resulting in unwanted indels. While BE4max targeting EMX1 in the nucleus resulted in 1.8±0.67% indels, typical of nuclear cytosine base editors, indels were not detected (<0.1%) at ND6 in HEK293T cells despite editing by ND6-DdCBE on both DNA strands (FIG. 25C). Similarly, in U20S cells, indels were not detected at ND6 while CCR5-DdCBE targeting nuclear CCR5 yielded 3.7±0.74% indels (FIGS. 35A-35B). Indeed, we observed remarkably high product purities—the proportion of edited alleles that contain only the targeted C·G-to-T·A conversions—for DdCBE-mediated mtDNA base editing of ND6 in HEK293T cells (99.9±0.007%) and U20S cells (99.5±0.22%), even beyond the product purities of BE4max (96±0.78%) and CCR5-targeting DdCBE (95±0.52%) when editing nuclear DNA (FIG. 25D and FIG. 35C).

Taken together, the very high product purity and lack of indels associated with DdCBE suggest that uracil repair processes that lead to indels and other byproducts in nuclear DNA³² are inefficient in the mitochondria. This model is consistent with previous observations that a ssDNA deaminase targeted to the mitochondria introduced C·G-to-T·A conversions with no indels⁶², supporting a model in mitochondria in which lesion-containing genomes are degraded rather than repaired^{63,64}, resulting in selective maintenance of mtDNA copies that have been cleanly edited.

We analyzed the ND6 allele distributions produced by ND6-DdCBE and found that alleles containing the G₆G7A-to-A₆A₇A conversion (which corresponds to a TC₇C₆-to-TT₇T₆ conversion in the complementary mtDNA strand) accounted for 38±0.90% of all edited alleles (FIG. 25E). Notably, AC₁₁T-to-AT₁₁T conversion was present only in alleles that already had edits at the more readily edited TCC contexts (FIG. 23I), suggesting that DddAₜₒₓ may operate processively. This behavior was not apparent in our bacterial genome mutational analyses, therefore it is likely to derive from tethering the enzyme to DNA (FIGS. 31A-31D).

Taken together, these findings establish a precise mtDNA editing capability that uses an unprecedented dsDNA-specific cytidine deaminase that we split to mitigate its toxicity, programmable dsDNA-binding TALE arrays, and a uracil glycosylase inhibitor to achieve efficient RNA-free base editing in the mitochondria.

Mitochondrial Base Editing of Five mtDNA Genes in Human Cells

To explore the generality of DdCBE for mtDNA editing, we constructed seven additional pairs of TALE array proteins either engineered de novo or adapted from validated mitoTALE arrays (Table 3) to target five mitochondrial genes: ND1, ND2, ND4, ND5, and ATP8. For some of the targeted sites, we expected DdCBE editing to install disease-relevant mutations (see the Supplementary Discussion section of Example 1). For example, C·G-to-T·A conversion by ND2- and ND4-DdCBE would install the m.5032G>A and m.11922G>A mutations, respectively, which are believed to be disease-causing in cancer-related tumors of the kidney and thyroid^{5, 65}.

Mitochondrial base editing efficiencies of DdCBEs in HEK293T cells 3-6 days after treatment varied between 4.6-49% depending on the split type, split orientation, and target cytidine position within the spacing region (FIGS. 26A-26J). For DdCBEs using the G1333 split, those with DddAₜₒₓ-C fused to the right-side mitoTALE (Right-G1333-DddAₜₒₓ-C+Left-G 1333-DddAₜₒₓ-N) resulted in 2.1- to 15-fold higher editing efficiencies than Right-G1333-DddAₜₒₓ-N+Left-G1333-DddAₜₒₓ-C, regardless of the spacing length and positions of TC target bases (FIGS. 26A-26E, and FIG. 26G).

In contrast, the effect of split orientation on editing efficiencies was more site-dependent for the G1397 split. The Right-G1397-DddAₜₒₓ-N+Left-G1397-DddAₜₒₓ-C orientation mediated 3.1- to 7.6-fold higher editing than the Right-G1397-DddAₜₒₓ-C+Left-G1397-DddAₜₒₓ-N orientation at TC₁₂C within an 18-bp spacing region (FIGS. 26D-26E), but was 3.4-fold less efficient at converting TC₁₁C within a 15-bp spacing region (FIG. 26F).

For a given TC target that was edited by both G1333 and G1397 splits, G1397 generally afforded higher editing efficiencies than G1333 (FIGS. 26A-26E). Collectively, optimized G1397-split DdCBEs mediated 42±2.5% average base editing efficiencies at four mtDNA sites (FIGS. 26A-26C, and FIG. 26E) and 9.0±0.92% average efficiencies at two other sites (FIGS. 26D and 26F), while the most efficient G1333-split DdCBEs yielded 43±2.2% average conversion at three sites (FIGS. 26A-26C) and 7.4±0.58% average efficiencies at three other sites (FIGS. 26D-26E, and FIG. 26G). In addition, we did not detect indels for the optimized G1333 and G1397 DdCBE orientations (FIGS. 45A-45B).

We observed a narrower editing window, which we define as the number of nucleotide positions upstream or downstream of the target TC base that are amenable to deamination, for G1397-split DdCBE compared to G1333-split DdCBE. The G1397 split efficiently converted TCs within a window of ˜1-2 nucleotides positioned (i) approximately 4-7 nucleotides from the 3′ end of a 15- to 18-bp spacing region in the H-strand of mtDNA (FIGS. 26A-26D, and FIG. 26F) or (ii) approximately 4 nucleotides from the 3′ end of a 15-18 bp spacing region in the L-strand of mtDNA (FIG. 26C). In contrast, the G1333 split converted TCs within a window of 3-5 nucleotides positioned approximately (i) 6-10 nucleotides from the 3′ end of a 15- to 18-bp spacing region in the H-strand of mtDNA (FIGS. 26A-26C) or (ii) approximately 4 nucleotides from the 5′ end of a 15- to 18-bp spacing region in the L-strand of mtDNA (FIG. 26A and FIG. 26G).

These results collectively suggest that each split is associated with a distinct preference for editing TCs within a specific editing window in the spacing region. We recommend testing G1397 and G1333 splits in both orientations to determine the fusion that gives the highest base editing efficiency for a given target sequence and spacing length.

To evaluate the durability of C·G-to-T·A mitochondrial DNA conversions in HEK293T cells, we tracked editing induced by ND6-, ND5.1-, ND5.2- and ATP8-DdCBE over 18 days at 3- or 6-day intervals, spanning approximately 21 cell divisions. Throughout this period, the viability of cells expressing DdCBEs remained indistinguishable from that of untreated cells (FIG. 36A). mtDNA editing did not produce large mtDNA deletions (FIG. 36B, FIG. 47 and the Supplementary Discussion section of Example 1) or alter mtDNA copy number (FIG. 36C).

Across all DdCBEs, editing efficiencies increased by 1.5- to 3.7-fold from day 3 to day 6 as levels of DdCBE protein persisted (FIGS. 37A-37F). DdCBE proteins were not detected by day 12 (FIG. 37F), but editing levels were generally maintained and sustained through the end of the experiment (day 18) (FIGS. 37A-37F and the Supplementary Discussion section of Example 1). By comparison, nuclear base editing of EMX1 by BE2 and BE4max peaked at day 3 and was generally maintained through day 18 (FIG. 37E and the Supplementary Discussion section of Example 1). We observed similar trends in the stability of C·G-to-T·A mtDNA edits in U20S cells (FIGS. 46A-46E).

We further characterized the ND4-DdCBE-edited HEK293T cells to evaluate the functional consequences of mtDNA editing of ND4. Following 8 days of passaging of three independently edited cell lines, we observed persistence of m.11922G>A heteroplasmic mutation in ND4 (FIG. 26H). Mitochondrial DNA homeostasis was intact based on assessment of mtDNA copy number and expression of mtDNA-encoded transcripts (FIGS. 38A-38B). In principle, the m.11922G>A ND4 mutation should disrupt complex I of the electron transport chain. Indeed, we observed decreased rates of oxidative phosphorylation (FIG. 26I), as well as basal and uncoupled respiration rates (FIG. 26J), in the edited cell lines compared to mock-edited cell lines.

Collectively, these results show that DdCBEs are capable of inducing durable, C·G-to-T·A conversions in mtDNA that are stably maintained (in the absence of edit-induced fitness changes) over many cell divisions without concomitant loss of cell viability and mtDNA copy number.

### Off-Target Editing by DdCBEs

Virtually all genome editing agents reported to date exhibit some degree of editing at off-target loci, a consequence of the inherently imperfect nature of binding specificity, as well as the high sensitivity of modern genome analyses^{1,2,66}. To profile potential off-target activity of DdCBE in the human mitochondrial genome, we transfected HEK293T cells with plasmids that constitutively expressed optimized DdCBE halves targeting ND6, ND5.1, ND5.2, ND4 or ATP8, or the corresponding inactive mutant DdCBE (dead-DdCBE) containing the E1347A mutation in DddAₜₒₓ. The dead-DdCBE controls enable identification of single-nucleotide variants (SNVs) that arise from background heteroplasmy to be distinguished from those that arise from off-target editing. To test for spontaneous assembly of split DddAₜₒₓ in the absence of TALE-directed DNA binding, cells were also transfected with plasmids expressing DddAₜₒₓ halves containing MTS-G1397 split DddAₜₒₓ-UGI, with no TALE array. Bulk cell populations were collected 3 days after transfection and sequenced by ATAC-seq⁶⁷⁻⁶⁹ to capture the entire 16.6-kb mitochondrial genome with an average of ˜5,100 to 9,900-fold coverage per mtDNA base (FIG. 27A and FIG. 47). High-confidence SNVs corresponded to >0.1% frequency in at least one biological replicate and were largely absent in the dead-DdCBE and untreated controls.

The average frequencies of genome-wide off-target C·G-to-T·A editing by ND5.2-DdCBE, ND4-DdCBE and ATP8-DdCBE were comparable to that of the untreated and TALE-free G1397 DddAₜₒₓ controls (0.030-0.034% for the DdCBEs versus 0.029-0.030% for the controls), while ND5.1-DdCBE had 1.6-fold higher average off-target editing frequency (0.049%) compared to the untreated control (FIG. 27B). In general, the average off-target editing frequencies for these standard DdCBEs (ND5.1, ND5.2, ND4 and ATP8) were 150- to 860-fold lower than the average on-target editing frequencies (FIGS. 39B-39E). Overall, these results indicate that DdCBEs exhibit high ratios of on-target to off-target editing.

ND6-targeting DdCBE showed 4.2-fold higher average off-target editing frequency (0.13%) compared to that of the untreated control (FIG. 27B). We attribute the unusually high number of average off-target C·G-to-T·A conversions from ND6-DdCBE to its mutant NTD, which was engineered to be permissive for any DNA base at position No (FIG. 25A). This mutant NTD may increase the non-specific binding activity of TALE array proteins, a feature of their two-state search mechanism that is distinct from site-specific DNA target recognition by TALE repeat domains⁷⁰.

Among the DdCBEs with standard NTDs, off-target editing levels did not strongly correlate with on-target editing efficiencies (compare FIG. 39B to FIG. 39D). Moreover, we observed that steady-state expression levels of DdCBEs are similar, and do not account for differences in DdCBE off-target activities (FIGS. 48A-48B and the Supplementary Discussion section of Example 1). The observations that off-target average editing frequencies and SNV numbers for TALE-free split G1937 DddAₜₒₓ are comparable to those of untreated controls (FIGS. 27B-27C) suggest that off-target activity resulting from spontaneous reassembly of split DddAₜₒₓ is negligible. Since all tested DdCBEs (with the exception of ND6-targeting DdCBE) share wild-type NTDs and the same deaminase domains, but contain different lengths and sequences of TALE repeat arrays, we conclude that TALE domains influence off-target DdCBE activity.

We noted a strong 5′-TC-3′ preference across all tested DdCBEs that matched the sequence preferences for overexpression of free DddAₜₒₓ in E. coli (compare FIG. 27D to FIG. 27I). More than 99% of the off-target SNVs in DdCBE-treated samples were C·G-to-T·A transitions (see Table 5 for list of all off-target SNVs, and the Supplementary Discussion section of Example 1) while SNVs in dead-DdCBE and untreated controls were a mixture of DNA transitions and transversions (FIG. 39F), suggesting that these off-target SNVs arise from DdCBE-mediated deamination rather than cell-specific heteroplasmy or somatic mutations.

To further probe the nature of off-target edits by DdCBEs, we searched for sequence homology between 20-bp regions flanking each off-target edit and on-target TALE-binding sites. We did not observe any consensus off-target sequences that closely resemble on-target TALE binding sites (FIG. 27D). In addition, we noted a high percentage of overlapping SNVs (22% for ND6-DdCBE and >40-80% for ND5.1-, ND5.2-, ND4- and ATP8-DdCBEs) across samples treated with DdCBEs containing distinct TALE arrays programmed to bind different on-target sites (FIG. 27E and Table 6). Collectively, these results suggest that DdCBE-mediated off-target editing is TALE-dependent, but does not arise from canonical DdCBE editing at sequences similar to the on-target sites. Instead, we speculate that different TALE arrays experience different frequencies of engaging random DNA sequences⁷⁰⁻⁷³, and DdCBEs that contain TALE arrays with higher non-specific DNA binding activity may induce higher frequencies of deamination at off-target sequences.

### DISCUSSION

This study describes the discovery of a dsDNA-specific cytidine deaminase toxin, and its development into an RNA-free base editor that can install the first targeted point mutations in the human mitochondrial genome. Additional research will be needed to fully elucidate the principles governing DdCBE efficiency and specificity. Given that DddAₜₒₓ activity can be attenuated by the DddI_{A} immunity protein, this built-in kill switch could be used to control DdCBE activity if necessary. In addition, developing in vitro and in vivo strategies to deliver DdCBEs will be essential for exploring their therapeutic potential in other cell types and in animal models of mitochondrial diseases. Evolving or engineering DddAₜₒₓ variants with altered sequence context preferences beyond 5′-TC-3′⁷⁴, or that deaminate nucleosides other than cytidine³⁵, would further expand the scope of mtDNA editing. The largely untapped natural diversity of bacterial DNA deaminases could provide additional starting points towards these and other novel base editing systems.

Editing of mitochondrial DNA has previously been limited to heteroplasmy shifts⁷⁵ or copy number modulation⁷⁶ due to the lack of DSB-initated DNA repair pathways found in the nucleus, and the unavailability of reliable methods to import guide RNAs required for CRISPR methods. Following our discovery of an interbacterial toxin DddAₜₒₓ that unprecedentedly deaminates cytidines only in dsDNA, we designed splits of DddAₜₒₓ to overcome its inherent toxicity and engineered fusions of the non-toxic halves to DNA-binding TALE proteins. The resulting DdCBEs enable programmable C·G-to-T·A conversions in mtDNA without requiring DSBs, a new capability that has the potential to install or correct pathogenic mtDNA SNPs associated with mitochondrial disorders (FIG. 50 and Table 7) and expand our knowledge about mitochondrial biology.

More broadly, the principles behind DdCBE demonstrate how enzymes that modify double-stranded DNA can be made dependent on DNA-binding proteins to enable efficient CRISPR-free gene editing systems. Finally, while this study has focused on the use of DdCBE for mitochondrial base editing, some features of DdCBE (or zinc-finger array variants), such as its all-protein nature, lack of a PAM requirement, and independence from CRISPR components, may also offer advantages for base editing outside the mitochondria.

### Methods

### Bacterial Strains and Culture Conditions

Except as noted, all bacterial strains used in this study were grown in Lysogeny Broth (LB) at 37° C. When required, media was supplemented with the following: carbenicillin 150 μg mL⁻¹ gentamycin μg mL⁻¹, 80 μM IPTG, 0.05% (w/v) rhamnose, chloramphenicol μg mL⁻¹ or tetracycline μg mL⁻¹ for E. coli, chloramphenicol 15 μg mL⁻¹ or gentamycin 30 μg mL⁻¹ for P. aeruginosa, and carbenicillin 150 μg mL⁻¹, or tetracycline 120 μg mL⁻¹ for B. cenocepacia. E. coli strains DH5α, XK1502, and BL21 were used for plasmid maintenance, toxicity and mutagenesis assays, and protein expression, respectively. P. aeruginosa strains were derived from the model strain PAO1, and B. cenocepacia strains were derived from the cystic fibrosis clinical isolate H111. A detailed description of the bacterial strains and plasmids used in this study is provided in Tables 8A-8B.

### Genetic Techniques and Plasmid Construction for Bacterial Expression

All procedures for DNA manipulation and transformation were performed with standard methods. Molecular biology reagents, Phusion® high fidelity DNA polymerase, restriction enzymes, UDG, and Gibson Assembly Reagent were obtained from New England Biolabs (NEB). GoTaq® Green Master Mix was obtained from Promega. Primers and gBlocks used in this study were obtained by Integrated DNA Technologies (IDT). A list of all primers is provided in the Supplementary Sequence section in Example 1.

Protein expression constructs were generated by Gibson assembly. For functional protein expression assays of DddAₜₒₓ, TadA and CDD, the relevant genes or gene fragments were amplified from B. cenocepacia (DddAₜₒₓ) or E. coli genomic DNA and cloned into the vector pSCRhaB2. DddA₁ was amplified from B. cenocepacia and cloned into pPSV39, and the expression construct for DddAₜₒₓ(E1347A) was generated by splicing by overlap extension PCR followed by Gibson assembly with pSCRhaB2. For the APOBEC3G expression construct, the gene sequence was codon optimized for expression in E. coli, generated by synthesis as a gBLOCK (IDT) and cloned into pSCRhaB2. For protein purification, DddAₜₒₓ and DddA₁ were amplified from B. cenocepacia and cloned into pETDuet.

Deletion of udg in P. aeruginosa was performed using allelic exchange mediated by the vector pEXG2, using counter selection with sucrose as described in detail previously⁷⁹. Gene deletions and nucleotide substitutions in B. cenocepacia were performed by homologous recombination using the plasmid pDONRPEX18Tp-SceI-pheS, followed by counter selection using the plasmid pDAI-SceI and plasmid curing using 0.1% (w/v) p-chlorophenylalanine, as described previously⁸⁰. Gentamycin resistant B. cenocepacia was generated by insertion of a resistance cassette at the Tn7 site attachment site as described previously⁸¹.

### Plasmid Construction for Mammalian Expression

PCR was performed using Phusion U Green Multiplex PCR Master Mix (ThermoFisher Scientific), Phusion U Green Hot Start DNA Polymerase (ThermoFisher Scientific) or Q5 Hot Start High-Fidelity DNA Polymerase (New England Biolabs). All plasmids were constructed using USER cloning (New England Biolabs). DddAₜₒₓ and mitoTALE genes were synthesized as gene blocks and codon optimized for human expression (Genscript). BE2 and BE4max plasmids were obtained from previous reports^{34,54}. DddAₜₒₓ-Cas9 fusions and DdCBE variants were cloned into pCMV (mammalian codon-optimized) backbones. sgRNA plasmids were constructed by blunt-end ligation of a linear polymerase chain reaction (PCR) product generated by encoding the 20- to 23-nt variable protospacer sequence onto the 5′ end of an amplification primer and treating the resulting piece with KLD Enzyme Mix (New England Biolabs) according to the manufacturer's instruction. Mach1 chemically competent E. coli cells (ThermoFisher Scientific) were used for plasmid construction. Plasmids for mammalian transfection were purified using ZymoPURE II Plasmid Midiprep Kits (Zymo Research), as previously described⁸². A list of all primers is provided in the Supplementary Sequence section of Example 1.

### Bacterial Competition Experiments

Donor and recipient strains were grown overnight and mixed in a 10:1 (v/v) ratio for donor and recipient, respectively. The cell suspensions were then concentrated to a total OD₆₀₀ of 10, and 10 μL was spotted on a 0.2 μm nitrocellulose membrane placed on LB with 3% (w/v) agar followed by 6 hours incubation at 37° C. After the incubation, cells were scraped from the membranes surface and resuspended in 1 mL LB. The initial donor:recipient ratio and the post-incubation ratio were determined by plating on LB agar (LBA) to determine the total number of colony forming units (CFU) and on LBA to with gentamycin to determine CFUs for the marked recipient strain. The competitive index is defined as the final donor:recipient ratio divided by the initial donor:recipient ratio. Competition experiments used to determine the bactericidal effect of DddA were performed similarly, but instead of cells being plated after 6 hours, they were harvested and plated hourly and CFU were enumerated on LBA with gentamycin to quantify the recipient strain population.

### Toxicity Assays

To evaluate the toxicity of deaminases expressed heterologously, overnight cultures of E. coli XK1502 containing the appropriate plasmids were diluted 1:1000 into fresh medium and grown until reaching exponential phase (OD₆₀₀ 0.6), at which point deaminase expression was induced with 0.2% (w/v) rhamnose. Aliquots of cultures were then collected periodically until 480 minutes of growth, and were diluted and plated onto LBA for CFU determination.

### Crystallization and Structure Determination

Crystals of the selenomethionine derivative hexahistidine-tagged DddAₜₒₓ(a.a. 1264-1427)·DddA₁ complex were obtained at 5-10 mg/mL in crystallization buffer (15 mM Tris pH 7.5, 150 mM NaCl, 1.0 mM tris(2-carboxyethyl)phosphine (TCEP)), mixed 1:1 with crystallization solution containing 25% (w/v) PEG 3350, 0.1 M Bis-Tris:HCl pH 6.5, 200 mM MgCl₂. Rectangular crystals grew to 400×200×100 μm over 5 days. Selenomethionine DddAₜₒₓ·DddA₁ crystals displayed the symmetry of space group P2₁2₁2 (a=126.8 Å, b=145.0 Å, c=64.2 Å, α=β=γ=90°), with four dimers in the asymmetric unit. Prior to data collection, crystals were cryoprotected in crystallization solution 15% glycerol, 25% PEG3350, 100 mM MgCl₂, 100 mM NaCl, 7.5 mM Tris pH 7.5, 50 mM Bis-Tris pH 6.5, 0.5 mM TCEP.

Highly redundant anomalous (SAD) data were obtained at 0.9790 A (peak) wavelength from a single selenomethionine crystal at 100 K temperature at the BL502 beamline (ALS, Lawrence Berkeley National Laboratory). Data were processed using HKL2000⁸³. Heavy atom searching using phenix.autosol identified 18 possible sites, and refinement yielded an estimated Bayes correlation coefficient of 55.9 to 2.5 Å resolution. After density modification, the estimated Bayes correlation coefficient increased to 61.2. Approximately 70% of the selenomethionine model was constructed automatically, and the remaining portion was built manually. The current model (Table 2) contains four DddA-DddIA dimers.

Refinement was carried out against peak anomalous data with Bijvoet pairs kept separate using phenix.refine⁸⁴ interspersed with manual model revisions using the program Coot⁸⁵ and consisted of conjugate-gradient minimization and calculation of individual atomic displacement and translation/libration/screw parameters⁸⁶. Residues that could not be identified in the electron density were: 1250-1289 and 1423-1427 for DddA, and 71-73 for DddIA. Both models exhibit excellent geometry, as determined by MolProbity⁸⁷. Ramachandran analysis identified 99.1% favored, 0.9% allowed, and 0% disallowed residues for the model. Coordinates and structure factors are deposited in the RCSB Protein Data Bank (ID 6U08).

### Mutation Frequency Determination and SNP Generation Assay

To determine the frequency of mutations induced by expression of DddAₜₒₓ and DddAₜₒₓ (E1347 Å), overnight cultures of E. coli containing the expression plasmids for these proteins together with the plasmid for expression of DddA₁ were diluted 1:1000 into fresh medium and grown until reaching exponential phase (OD₆₀₀ 0.6). The cultures were then induced with 0.08 mM IPTG for DddIA and 0.04% rhamnose for DddAₜₒₓ or DddAₜₒₓ(E1347 Å) expression respectively. The combined expression of both toxin and immunity proteins at this low level allows the cells expressing DddAₜₒₓ to suffer growth arrest but does not result in a decrease in culture viability. After 1 hour under these inducing conditions, cultures were supplemented with 1 mM of IPTG to increase DddI_{A} expression and thus block DddA toxicity and were then grown an additional 16 hours. After this recovery period, the cultures were plated onto LBA containing rifampicin or no antibiotics. Mutation frequency was determined by the ratio of the number of rifampicin resistant colonies by the total CFU obtained on non-selective medium.

For the genome-wide identification of SNPs that accumulate following low level expression of DddAₜₒₓ or DddAₜₒₓ(E1347 Å), E. coli Δudg carrying plasmids for expression of one of these proteins plus the plasmid for expressing DddI_{A} was submitted to seven rounds of expression and recovery as described above, with cultures being plated after recovery and single colonies being selected and used to inoculate the subsequent round of expression. Randomly chosen single colonies were used to avoid introducing selection for increased fitness under the culture conditions⁸⁷. Five isolated colonies from each starting population subjected to this regimen were selected for whole genome sequencing.

Western Blot for E. coli-Expressed Deaminase

Western blotting to detect deaminases expressed in E. coli was performed using rabbit α-VSV-G (diluted 1:5000, Sigma) and detected with α-rabbit horseradish peroxidase-conjugated secondary antibodies (diluted 1:5000, Sigma). Loading control was performed with mouse α-RNAP (diluted 1:500, Biolegend) and detected with sheep α-mouse (diluted 1:500, Millipore). Western blots were developed using chemiluminescent substrate (SuperSignal West Pico Substrate, Thermo Scientific) and imaged with a C600 imager (Azure biosystems).

### Western Blot for Mammalian Cell-Expressed DdCBE

HEK293T cells were transfected as described below. For preparation of cell lysate for western blot analysis of DdCBE, cells were lysed in 150 L of ice-cold 1× RIPA buffer (Sigma) with added protease inhibitor (Roche Complete Mini) by incubating for 30 min at 4° C. with agitation. Lysates were cleared by pelleting at 12,000 rcf for 10 min at 4° C.

60 μL of cleared lysate supernatant was added to 20 μL of 4× LDS sample loading buffer (ThermoFisher Scientific) with a final DTT (Sigma Aldrich) concentration of 10 mM. Lysates were boiled for 10 min at 95° C. 15-20 μL of protein lysate was loaded into the wells of a Bolt 4-12% Bis-Tris Plus (Thermo Fisher Scientific) pre-cast gel. 6 μL of Precision Plus Protein Dual Color Standard (Bio-Rad) was used as a reference. Samples were separated by electrophoresis at 180 V for 35 min in Bolt MES SDS running buffer (Thermo Fisher Scientific). Transfer to a PVDF membrane was performed using an iBlot 2 Gel Transfer Device (Thermo Fisher Scientific) according to manufacturer's protocols. The membrane was blocked in Odyssey Blocking Buffer (LI-COR) for 1 h at room temperature, then incubated with rat anti-FLAG (ThermoFisher Scientific MA1-142; 1:2000 dilution), mouse anti-HA (ThermoFisher Scientific 26183; 1:2000 dilution) and rabbit anti-actin (CST 4970; 1:2000 dilution) in blocking buffer (0.5% Tween-20 in 1×PBS, 0.2 μm filtered) overnight at 4° C. The membrane was washed 3× with TBST (1×TBS in 0.5% Tween-20) for 10 min each at room temperature, then incubated with IRDye-labeled secondary antibodies goat anti-rat 680RD (LI-COR 926-68076), goat anti-mouse 800CW (LI-COR 926-32210) and donkey anti-rabbit 800CW (LI-COR 926-32213) diluted 1:5000 in blocking buffer for 1 h at room temperature. The membrane was washed as before, then imaged using an Odyssey Imaging System (LI-COR).

### Purification of Proteins for Biochemical Assays

Overnight cultures of E. coli BL21 pETDuet-1::dddAₜₒₓ-dddA₁, or E. coli BL21 pETDuet-1::dddAₜₒₓ (E1347 Å) were used to inoculate 2 L of LB broth in a 1:100 dilution and cultures were grown to approximately OD₆₀₀ 0.6. At this point, plasmid expression was induced with 0.5 mM IPTG and the cultures were incubated for 16 hours at 18° C. in a shaking incubator. Cell pellets were harvested by centrifugation at 4000 g for 20 min, followed by resuspension in 50 mL of lysis buffer (50 mM Tris-HCl pH 7.5, 500 mM NaCl, 30 mM imidazole, 1 mM DTT, and 1 mg/mL lysozyme). Cell pellets were then lysed by sonication (5 pulses, 10 s each) and supernatant was separated by centrifugation at 25,000 g for 30 min.

The DddAₜₒₓ-DddA₁ complex or DddAₜₒₓ(E1347 Å) was purified from cell lysates by nickel affinity chromatography using 4 mL of Ni-NTA agarose beads loaded onto a gravity-flow column. Supernatant was loaded onto the column and resin was washed with 50 mL of wash buffer (50 mM Tris-HCl pH 7.5, 500 mM NaCl, 30 mM imidazole, 1 mM DTT). Proteins of interest were eluted with 5 mL elution buffer (50 mM Tris-HCl pH 7.5, 300 mM Imidazole, 500 mM NaCl, 30 mM imidazole, 1 mM DTT). When DddAₜₒₓ(E1347 Å) was purified, the eluted samples were applied directly to size exclusion chromatography. For DddA-DddA₁, the eluted samples underwent a denaturation and renaturation step to isolate only the toxin. In this case, the eluted proteins were added to 50 mL 8 M urea denaturing buffer (50 mM Tris-HCl pH 7.5, 300 mM Imidazole, 500 mM NaCl, and 1 mM DTT) and incubated for 16 hours at 4° C. The 8 M urea denaturing buffer with the eluted proteins was loaded on a gravity-flow column with 4 mL Ni-NTA agarose beads. The column was washed with 50 mL 8 M urea denaturing buffer to remove any remaining DddA₁. While still bound to Ni-NTA agarose beads, DddAₜₒₓ was renatured by sequential washes with 25 mL denaturing buffer with decreasing concentrations of urea (6 M, 4 M, 2 M, 1 M), and a last wash with wash buffer to remove remaining traces of urea. Proteins bound to the column were then eluted with 5 mL elution buffer. The eluted samples were purified again by sizing exclusion chromatography using protein liquid chromatography (FPLC) with gel filtration on a Superdex200 column (GE Healthcare) in sizing buffer (20 mM Tris-HCl pH 7.5, 200 mM NaCl, 1 mM DTT, 5% (w/v) glycerol). The fraction purity was evaluated by SDS-PAGE gel stained with Coomassie Brilliant Blue and the highest quality factions were stored at −80° C.

### DNA Deamination Assays

All the DNA substrates were designed with the addition of a 5′ 6-FAM fluorophore for visualization, and they were purchased from Integrated DNA Technologies (IDT). All substrate sequences are present in the Supplementary Sequence section of Example 1. Reactions were performed in 10 μL of deamination buffer (20 mM MES pH 6.4, 200 mM NaCl, 1 mM DTT, 8% Ficoll 70, and 1 μM substrate) with APOBEC3A, DddAₜₒₓ or DddAₜₒₓ(E1347 Å) at the concentrations indicated in FIG. 1. Reactions were incubated for 1 hour at 37° C., followed by the addition 5 μL of UDG solution (New England Biolabs, 0.02 U/μL UDG in 1× UDG buffer) and further incubation for 30 minutes. Cleavage of substrates was induced by addition of 100 mM NaOH and incubation at 95° C. for 3 minutes. Samples were analyzed by denaturing 15% acrylamide gel electrophoresis and the resulting fluorescent DNA fragments were detected by imaged with a C600 (Azure biosystems).

### Poisoned Primer Extension Assay for RNA Deamination

All substrate sequences are listed in the Supplementary Sequence section of Example 1. The RNA substrates and the oligonucleotide containing a 5′ 6-FAM fluorophore for visualization were purchased from Integrated DNA Technologies (IDT). Deamination reactions were performed in 10 μL of RNA deamination buffer (Tris-HCl pH 7.5, 200 mM NaCl, 1 mM DTT) with the addition of 1 μM of DddAₜₒₓ or DddAₜₒₓ (E1347 Å). Substrate combinations and concentrations were added as indicated in FIGS. 29A-29B, and reactions were incubated for 1 hour at 37° C. cDNA synthesis was performed in a 10-μL reaction (2.5 U/μL MultiScribe™ Reverse Transcriptase (ThermoFisher), 1 μL deamination reaction, 1.5 μM oligonucleotide, 100 μM dATP, 100 μM dCTP, 100 μM dTTP, and 100 μM ddGTP). The reaction was incubated at 37° C. for 10 minutes and samples were analyzed by denaturing 15% acrylamide gel electrophoresis. The synthesized cDNA fragments were detected by fluorescence imaging with a C600 (Azure biosystems).

### Genome Sequencing and SNP Identification in Bacteria

Overnight cultures from isolated colonies were used for total gDNA extraction with the DNeasy Blood & Tissue kit (Qiagen), and extraction yield was quantified using a Qubit. Sequencing libraries were constructed using the Nextera DNA Flex Library Prep Kit (Illumina). Library quality and concentration was evaluated with a Qubit and TapeStation System (Agilent). Sequencing was performed with an Illumina MiSeq instrument (300 cycles paired end program). Genome mapping was performed with BWA⁸⁸ using the E. coli MG1655 (NC_000913.3) genome as a reference. Pileup data from alignments were generated with SAMtools and variant calling was performed with VarScan2⁸⁹. SNPs were considered valid if they were present at a frequency higher than 90%.

### Mammalian Cell Culture

All cells were cultured and maintained at 37° C. with 5% CO₂. Antibiotics were not used for cell culture. HEK293T cells [CRL-3216, American Type Culture Collection (ATCC)] were cultured in Dulbecco's modified Eagle's medium plus GlutaMax (Thermo Fisher Scientific) supplemented with 10% (v/v) fetal bovine serum (FBS). U20S cells (HTB-96, ATCC) were cultured in MyCoy's 5 Å medium plus GlutaMax (Thermo Fisher Scientific) supplemented with 10% (v/v) FBS. HeLa cells (ATCC CCL-2) were cultured in high glucose DMEM (Gibco) with 10% fetal bovine serum (Atlanta Biological), and 100 U/mL penicillin (Sigma-Aldrich). Cell lines were authenticated by their respective suppliers and tested negative for mycoplasma.

### Mammalian Cell Lipofection

HEK293T cells were seeded on 48-well collagen-coated plates (Corning) at a density of 2×10⁵ cells/mL 18-24 hours before lipofection. Lipofection was performed at a cell density of approximately 70%. For split DddAₜₒₓ-Cas9 screening, cells were transfected with 375 ng of split DddAₜₒₓ-dSpCas9 monomer expression plasmid, 375 ng of split DddAₜₒₓ-SaKKH-Cas9(D10 Å) monomer expression plasmid, 125 ng of SpCas9 gRNA expression plasmid and 125 ng of SaKKH gRNA plasmid. pUC19 was used as a filler DNA for monomer and no-gRNA control experiments to make up to 1000 ng of total plasmid DNA. For DdCBE experiments, cells were transfected with 500 ng of each mitoTALE monomer to make up 1000 ng of total plasmid DNA. Lipofectamine 2000 (1.5 μL; ThermoFisher Scientific) was used per well. Cells were harvested at the indicated timepoint.

For western blot analysis of DdCBEs expressed in mammalian cells, HEK293T cells were seeded on 6-well tissue culture-treated plates (Corning) at a density of 2×10⁵ cells/mL 18-24 hours before lipofection. Cells were transfected with 4000 ng of each mitoTALE monomer to make up 8000 ng of total plasmid DNA. Lipofectamine 2000 (12 μL; ThermoFisher Scientific) was used per well. Cells were harvested at the indicated timepoint.

For inducible expression of full-length DddAₜₒₓ fused to Cas9 (FIGS. 40A-40C), doxycycline hyclate (Millipore Sigma) was freshly prepared and added directly to cells ˜4-5 h after transfection to a final concentration of 0.1 μg/mL.

### Mammalian Cell Nucleofection

We combined 500 ng of Left DdCBE monomer and 500 ng of Right DdCBE monomer in a volume that did not exceed 2 μL. This combined plasmid mixture was nucleofected in a final volume of 22 μL per sample in a 16-well Nucleocuvette strip (Lonza). U20S cells were nucleofected using the SE Cell Line 4D-Nucleofector X Kit (Lonza) with 30,000-50,000 cells per sample (program DN-100), according to the manufacturer's protocol.

### Cell Viability Assays

Cell viability was measured every either every 24 h post-transfection for 3 days (FIG. 40B) or every 3 to 6-days over an 18-day time course (FIG. 36A) using the CellTiter-Glo 2.0 assay (Promega) according to the manufacturer's protocol. Luminescence was measured in 96-well flat black-bottomed polystyrene microplates (Corning) using a M1000 Pro microplate reader (Tecan) with a 1-s integration time.

Genomic DNA Isolation from Mammalian Cell Culture

Medium was removed, and cells were washed once with 1× Dulbecco's phosphate-buffered saline (ThermoFisher Scientific). Genomic DNA extraction was performed by addition of 45 μL freshly prepared lysis buffer (10 mM tris-HCl (pH 7.0), 0.05% SDS, and proteinase K (20 μg/mL; ThermoFisher Scientific)) directly into the 48-well culture well. The extraction solution was incubated at 37° C. for 60 min and then 80° C. for 20 min. Resulting genomic DNA was subjected to bead cleanup with AMPure DNAdvance beads according to manufacturer's instructions (Beckman Coulter A48705).

### Determination of Relative Total Mitochondrial DNA Levels by Quantitative PCR

Quantitative PCR (qPCR) reactions were performed on a Bio-Rad CFX96/C1000 qPCR machine performed using SYBR green (Lonza). 5 ng of purified DNA was used as template input in a 25 μL reaction volume. For all reactions, the protocol used was an initial heating step of 2 min at 98° C. followed by 40 cycles of amplification (10 s at 98° C., 20 s at 62° C., 15 s at 72° C.). Single threshold values (AC) were determined by manufacturer's software. The level of mtDNA was determined by the calculating the ratio of total mtDNA to genomic DNA (β-actin)

$$( Ratio = \frac{{( {E}_{mtDNA} )}^{Δ {C}_{mtDNA} ( DdCBE - dead DdCBE )}}{{( {E}_{β - actin} )}^{Δ {C}_{β - actin} ( DdCBE - dead DdCBE )}} ,$$

where E is the efficiency of the qPCR reaction; E_{ND6}=0.858, E_{ND5}=0.844, E_{ATP8}=0.995, E_{β-actin}=1.05). Refer to the Supplementary Sequence section of Example 1 for list of primers used. NC_012920 was used as the reference for mtDNA; NG_003019 was used as the reference for human ACTBP2.
Long-Range PCR to Detect mtDNA Deletions

Long-range PCR was performed on purified genomic DNA as previously with listed primers (Supplementary Sequence section of Example 1) to capture the whole mtDNA genome as two overlapping fragment of ˜8 kb each. Briefly, ˜50-200 ng of purified DNA was used as input for amplification by PRIMESTAR GXL DNA polymerase (Takara). For all reactions, the protocol used was an initial heating step of 1 min at 94° C. followed by 30 cycles of amplification (30 s at 98° C., 30 s at 60° C., 9 min at 72° C.). Unpurified PCR products were run on 0.8% agarose gel and stained with ethidium bromide.

### Immunocytochemical Studies of DdCBE Localization

HeLa cells were transfected with a total of 1 μg of plasmid DNA to express left (HA-tagged) or right (FLAG-tagged) monomers of each DdCBE using Lipofectamine 3000 (Invitrogen) according to the manufacturer's protocol. After 24 hours incubation, cells were labelled with MitoTracker Deep Red (ThermoFisher) at a final concentration of 100 nM for 30 minutes at 37° C., 5% CO₂ incubator. Cells were then seeded on an 8-well chamber glass slide (Ibidi) and fixed in 4% paraformaldehyde/PBS for 15 minutes at room temperature. Next, cells were washed twice with PBS and permeabilized in PBS containing 0.1% saponin and 1% BSA for 30 minutes at room temperature. Cells were then immunostained with α-HA (Biolegend) or α-Flag (Sigma Aldrich), followed by Alexa-fluor conjugated α-mouse (HA tag) or α-rabbit (FLAG tag) secondary antibodies (Thermo Fisher). Images were taken using a 60× objective with the high-resolution widefield Nikon system. Acquired images were processed in Fiji (http://fiji.sc/).

### High-Throughput DNA Sequencing of Genomic DNA Samples

Genomic sites of interest were amplified from genomic DNA samples and sequenced on an Illumina MiSeq as previously described with the following modifications⁴³. Amplification primers containing Illumina forward and reverse adapters (Supplementary Sequence section of Example 1) were used for a first round of PCR (PCR 1) to amplify the genomic region of interest. Briefly, 1 μL of purified genomic DNA was input into the first round of PCR (PCR1). For PCR1, DNA was amplified to the top of the linear range using Phusion Hot Start II High-Fidelity DNA Polymerase (ThermoFisher Scientific), according to the manufacturer's instructions but with the addition of 0.5× SYBR Green Nucleic Acid Gel Stain (Lonza) in each 25-μL reaction. For all amplicons, the PCR1 protocol used was an initial heating step of 2 min at 98° C. followed by an optimized number of amplification cycles (10 s at 98° C., 20 s at 62° C., 30 s at 72° C.). Quantitative PCR was performed to determine the optimal cycle number for each amplicon. The number of cycles needed to reach the top of the linear range of amplification are ˜27-28 cycles for nuclear DNA amplicons and ˜17-19 cycles for mtDNA amplicons. Barcoding PCR2 reactions (25 μL) were performed with 1 μL of unpurified PCR1 product and amplified with Q5 Hot Start MasterMix (New England Biolabs) using the following protocol 98° C. for 2 min, then 12 cycles of [98° C. for 10 s, 61° C. for 20 s, and 72° C. for 30 s], followed by a final 72° C. extension for 2 min. PCR products were evaluated analytically by electrophoresis in a 1.5% agarose gel. After PCR2, up to 240 samples with different barcode combinations were combined and purified by gel extraction using the QIAquick Gel Extraction Kit (QIAGEN). DNA concentration was quantified using the Qubit ssDNA HS Assay Kit (Thermo Fisher Scientific) to make up a 4 nM library. The library concentration was further verified by qPCR (KAPA Library Quantification Kit-Illumina, KAPA Biosystems) and sequenced using an Illumina MiSeq with 210- to 280-bp single-end reads.

### Analysis of HTS Data for DNA Sequencing and Targeted Amplicon Sequencing

Sequencing reads were demultiplexed using MiSeq Reporter (Illumina). Batch analysis with CRISPResso2⁹⁰ was used for targeted amplicon and DNA sequencing analysis. A 10-bp window was used to quantify indels centered around the middle of the dsDNA spacing. To set the cleavage offset, a hypothetical 15- or 16-bp spacing region has a cleavage offset of −8. Otherwise, the default parameters were used for analysis. The output file “Reference.NUCLEOTIDE_PERCENTAGE_SUMMARY.txt” was imported into Microsoft Excel for quantification of editing frequencies. Reads containing indels within the 10-bp window are excluded for calculation of editing frequencies. The output file “CRISPRessoBatch_quantification_of_editing_frequency.txt” was imported into Microsoft Excel for quantification of indel frequencies. Indel frequencies were computed by dividing the sum of Insertions and Deletions over the total number of aligned reads.

### Bulk ATAC-Seg for Whole Mitochondrial Genome Sequencing

ATAC-seq was performed as previously described⁶⁷. In brief, 5,000-10,000 cells were trypsinzed, washed with PBS, pelleted by centrifugation and lysed in 50 μL of lysis buffer (0.1% Igepal CA-360 (v/v %), 10 mM Tris-HCl, 10 mM NaCl and 3 mM MgCl2 in nuclease-free water). Lysates were incubated on ice for 3 minutes, pelleted at 500 rcf for 10 minutes at 4° C. and tagmented with 2.5 μL of Tn5 transposase (Illumina #15027865) in a total volume of 10 p L containing 1×TD buffer (Illumina #15027866), 0.1% NP-40 (Sigma), and 0.3× PBS. Samples were incubated at 37° C. for 30 minutes on a thermomixer at 300 rpm. DNA was purified using the MinElute PCR Kit (Qiagen) and eluted in 10 μL elution buffer. All 10 μL of the eluate was amplified using indexed primers (1.25 μM each) listed in the the Supplementary Sequence section of Example land NEBNext High-Fidelity 2× PCR Master Mix (New England Biolabs) in a total volume of 50 μL using the following protocol 72° C. for 5 min, 98° C. for 30 s, then 5 cycles of [98° C. for 10 s, 63° C. for 30 s, and 72° C. for 60 s], followed by a final 72° C. extension for 1 min. After the initial 5 cycles of pre-amplification, 5 μL of partially amplified library was used as input DNA in a total volume of 15 μL for quantitative PCR using SYBR Green to determine the number of additional cycles needed to reach ⅓ of the maximum fluorescence intensity. Typically, 3-8 cycles were conducted on the remaining 45 μL of partially amplified library. The final library was purified using a MinElute PCR kit (Qiagen) and quantified using a Qubit dsDNA HS Assay kit (Invitrogen) and a High Sensitivity DNA chip run on a Bioanalyzer 2100 system (Agilent). All libraries were sequenced using Nextseq High Output Cartridge kits on an Illumina Nextseq 500 sequencer. Libraries were sequenced using paired-end 2×75 cycles and demultiplexed using the bc12fastq program.

### Genome Sequencing and SNP Identification in Mitochondria

SNP identification in mitochondria was performed similarly to in bacteria, with the following modifications. Genome mapping was performed with BWA (v0.7.17) using NC_012920 genome as a reference. Duplicates were marked using Picard tools (v2.20.7). Pileup data from alignments were generated with SAMtools (v1.9) and variant calling was performed with VarScan2 (v2.4.3). Variants that were present at a frequency greater than 0.1% and a p-value less than 0.05 (Fisher's Exact Test) were called as high-confidence SNPs independently in each biological replicate. Only reads with Q>30 at a given position were taken into account when calling SNPs at that particular position.

### Calculation of Average Off-Target C·G-to-T·A Editing Frequency

To calculate the mitochondrial genome-wide average off-target editing frequency for each DdCBE in FIG. 27B, REDItools was used (v1.2.1) (Diroma et al., Brief Bioinform. 20, 436-447 (2019)). All nucleotides except cytidines and guanosines were removed and the number of reads covering each C·G base pair with a PHRED quality score greater than 30 (Q>30) was calculated. The on-target C·G base pairs (depending on the DdCBE used in each treatment) were excluded in order to only consider off-target effects. C·G-to-T·A SNVs present at high frequencies (>50%) in both treated and untreated samples (that therefore did not arise from DdCBE treatment) were also excluded. The average off-target editing frequency was then calculated independently for each biological replicate of each treatment condition as: (number of reads in which a C·G base pair was called as a T·A base pair, summed over all non-target C·G base pairs)+(total number of reads that covered each non-target C·G base pair). Sequence logos in FIG. 27D depicting the local sequence context of all off-target SNVs were generated as described previously⁹¹. For FIGS. 39A-39E, the average frequency of each SNV was calculated by taking the average of three frequencies from the biological triplicates.

### Effect Prediction of the C·G-to-T·A Off-Target SNVs Identified by ATAC-Seq

SIFT (https://sift.bii.α-star.edu.sg/) was used to predict the outcome of nonsynonymous mutations on protein function. High- and low-confidence calls were made using standard SIFT parameters with GRCh37.74 database as the reference genome.

### Data Availability

High-throughput sequencing and whole-mitochondria sequencing data is deposited in the NCBI Seqeunce Read Archive (PRJNA603010). Amino acids sequences of all base editors in this study are provided in the Supplementary Sequences section of Example 1.

### REFERENCES FOR EXAMPLE 1

1 Komor, A. C., Badran, A. H. & Liu, D. R. CRISPR-Based Technologies for the Manipulation of Eukaryotic Genomes. Cell 169, 559, doi:10.1016/j.cell.2017.04.005 (2017).
2 Rees, H. A. & Liu, D. R. Base editing: precision chemistry on the genome and transcriptome of living cells. Nat Rev Genet 19, 770-788, doi:10.1038/s41576-018-0059-1 (2018).
3 Iyer, L. M., Zhang, D., Rogozin, I. B. & Aravind, L. Evolution of the deaminase fold and multiple origins of eukaryotic editing and mutagenic nucleic acid deaminases from bacterial toxin systems. Nucleic acids research 39, 9473-9497, doi:10.1093/nar/gkr691 (2011).
4 Vafai, S. B. & Mootha, V. K. Mitochondrial disorders as windows into an ancient organelle. Nature 491, 374-383, doi:10.1038/nature11707 (2012).
5 Gopal, R. K. et al. Widespread Chromosomal Losses and Mitochondrial DNA Alterations as Genetic Drivers in Hurthle Cell Carcinoma. Cancer Cell 34, 242-255.e245, doi:doi.org/10.1016/j.ccell.2018.06.013 (2018).
6 Schon, E. A. & Przedborski, S. Mitochondria: the next (neurode)generation. Neuron 70, 1033-1053, doi:10.1016/j.neuron.2011.06.003 (2011).
7 Ryzhkova, A. I. et al. Mitochondrial diseases caused by mtDNA mutations: a mini-review. Ther Clin Risk Manag 14, 1933-1942, doi:10.2147/TCRM.S154863 (2018).
8 Craven, L., Alston, C. L., Taylor, R. W. & Turnbull, D. M. Recent Advances in Mitochondrial Disease. Annual Review of Genomics and Human Genetics 18, 257-275, doi:10.1146/annurev-genom-091416-035426 (2017).
9 Gammage, P. A., Moraes, C. T. & Minczuk, M. Mitochondrial Genome Engineering: The Revolution May Not Be CRISPR-Ized. Trends Genet 34, 101-110, doi:10.1016/j.tig.2017.11.001 (2018).
10 Youssoufian, H. & Pyeritz, R. E. Mechanisms and consequences of somatic mosaicism in humans. Nat Rev Genet 3, 748-758, doi:10.1038/nrg906 (2002).
11 Bacman, S. R., Williams, S. L., Pinto, M., Peralta, S. & Moraes, C. T. Specific elimination of mutant mitochondrial genomes in patient-derived cells by mitoTALENs. Nat Med 19, 1111-1113, doi:10.1038/nm.3261 (2013).
12 Hashimoto, M. et al. MitoTALEN: A General Approach to Reduce Mutant mtDNA Loads and Restore Oxidative Phosphorylation Function in Mitochondrial Diseases. Mol Ther 23, 1592-1599, doi:10.1038/mt.2015.126 (2015).
13 Bacman, S. R. et al. MitoTALEN reduces mutant mtDNA load and restores tRNA(Ala) levels in a mouse model of heteroplasmic mtDNA mutation. Nat Med 24, 1696-1700, doi:10.1038/s41591-018-0166-8 (2018).
14 Reddy, P. et al. Selective Elimination of Mitochondrial Mutations in the Germline by Genome Editing. Cell 161, 459-469, doi:doi.org/10.1016/j.cell.2015.03.051 (2015).
15 Yang, Y. et al. Targeted elimination of mutant mitochondrial DNA in MELAS-iPSCs by mitoTALENs. Protein & cell 9, 283-297, doi:10.1007/s13238-017-0499-y (2018).
16 Pereira, C. V. et al. mitoTev-TALE: a monomeric DNA editing enzyme to reduce mutant mitochondrial DNA levels. EMBO Mol Med 10, doi:10.15252/emmm.201708084 (2018).
17 Kazama, T. et al. Curing cytoplasmic male sterility via TALEN-mediated mitochondrial genome editing. Nat Plants 5, 722-730, doi:10.1038/s41477-019-0459-z (2019).
18 Gammage, P. A., Rorbach, J., Vincent, A. I., Rebar, E. J. & Minczuk, M. Mitochondrially targeted ZFNs for selective degradation of pathogenic mitochondrial genomes bearing large-scale deletions or point mutations. EMBO Mol Med 6, 458-466, doi:10.1002/emmm.201303672 (2014).
19 Gammage, P. A. et al. Near-complete elimination of mutant mtDNA by iterative or dynamic dose-controlled treatment with mtZFNs. Nucleic Acids Res 44, 7804-7816, doi:10.1093/nar/gkw676 (2016).
20 Gammage, P. A. et al. Genome editing in mitochondria corrects a pathogenic mtDNA mutation in vivo. Nat Med 24, 1691-1695, doi:10.1038/s41591-018-0165-9 (2018).
21 Nissanka, N., Bacman, S. R., Plastini, M. J. & Moraes, C. T. The mitochondrial DNA polymerase gamma degrades linear DNA fragments precluding the formation of deletions. Nat Commun 9, 2491, doi:10.1038/s41467-018-04895-1 (2018).
22 Moretton, A. et al. Selective mitochondrial DNA degradation following double-strand breaks. PLoS One 12, e0176795, doi:10.1371/journal.pone.0176795 (2017).
23 Peeva, V. et al. Linear mitochondrial DNA is rapidly degraded by components of the replication machinery. Nat Commun 9, 1727, doi:10.1038/s41467-018-04131-w (2018).
24 Carelli, V., Giordano, C. & d'Amati, G. Pathogenic expression of homoplasmic mtDNA mutations needs a complex nuclear& #x2013;mitochondrial interaction. Trends in Genetics 19, 257-262, doi:10.1016/S0168-9525(03)00072-6 (2003).
25 Krishnan, K. J. et al. What causes mitochondrial DNA deletions in human cells? Nature Genetics 40, 275-279, doi:10.1038/ng.f.94 (2008).
26 Wallace, D. C. & Chalkia, D. Mitochondrial DNA Genetics and the Heteroplasmy Conundrum in Evolution and Disease. Cold Spring Harbor Perspectives in Biology 5, doi:10.1101/cshperspect.a021220 (2013).
27 Coulthurst, S. The Type VI secretion system: a versatile bacterial weapon. Microbiology (Reading, England) 165, 503-515, doi:10.1099/mic.0.000789 (2019).
28 Hood, R. D. et al. A type VI secretion system of Pseudomonas aeruginosa targets a toxin to bacteria. Cell host & microbe 7, 25-37 (2010).
29 Chen, J. & MacCarthy, T. The preferred nucleotide contexts of the AID/APOBEC cytidine deaminases have differential effects when mutating retrotransposon and virus sequences compared to host genes. PLoS Comput Biol 13, e1005471, doi:10.1371/journal.pcbi.1005471 (2017).

30 Richardson, S. R., Narvaiza, I., Planegger, R. A., Weitzman, M. D. & Moran, J. V. APOBEC3A deaminates transiently exposed single-strand DNA during LINE-1 retrotransposition. Elife 3, e02008, doi:10.7554/eLife.02008 (2014).
31 Matthews, M. M. et al. Structures of human ADAR2 bound to dsRNA reveal base-flipping mechanism and basis for site selectivity. Nature structural & molecular biology 23, 426-433, doi:10.1038/nsmb.3203 (2016).
32 Krokan, H. E. & Bjsris, M. Base Excision Repair. Cold Spring Harbor Perspectives in Biology 5, doi:10.1101/cshperspect.a012583 (2013).
33 Bhagwat, A. S. et al. Strand-biased cytosine deamination at the replication fork causes cytosine to thymine mutations in <em>Escherichia coli</em>. Proceedings of the National Academy of Sciences 113, 2176-2181, doi:10.1073/pnas.1522325113 (2016).
34 Komor, A. C., Kim, Y. B., Packer, M. S., Zuris, J. A. & Liu, D. R. Programmable editing of a target base in genomic DNA without double-stranded DNA cleavage. Nature 533, 420, doi:10.1038/nature17946 nature.com/articles/nature17946 #supplementary-information (2016).
35 Gaudelli, N. M. et al. Programmable base editing of A·T to G·C in genomic DNA without DNA cleavage. Nature 551, 464, doi:10.1038/nature24644 nature.com/articles/nature24644 #supplementary-information (2017).
36 Klug, A. The Discovery of Zinc Fingers and Their Applications in Gene Regulation and Genome Manipulation. Annual Review of Biochemistry 79, 213-231, doi:10.1146/annurev-biochem-010909-095056 (2010).
37 Joung, J. K. & Sander, J. D. TALENs: a widely applicable technology for targeted genome editing. Nat Rev Mol Cell Biol 14, 49-55, doi:10.1038/nrm3486 (2013).
38 Urnov, F. D., Rebar, E. J., Holmes, M. C., Zhang, H. S. & Gregory, P. D. Genome editing with engineered zinc finger nucleases. Nat Rev Genet 11, 636-646, doi:10.1038/nrg2842 (2010).
39 Jinek, M. et al. A programmable dual-RNA-guided DNA endonuclease in adaptive bacterial immunity. Science 337, 816-821, doi:10.1126/science.1225829 (2012).
40 Qi, L. S. et al. Repurposing CRISPR as an RNA-guided platform for sequence-specific control of gene expression. Cell 152, 1173-1183, doi:10.1016/j.cell.2013.02.022 (2013).
41 Kleinstiver, B. P. et al. Broadening the targeting range of Staphylococcus aureus CRISPR-Cas9 by modifying PAM recognition. Nat Biotechnol 33, 1293-1298, doi:10.1038/nbt.3404 (2015).
42 Nishimasu, H. et al. Crystal Structure of Staphylococcus aureus Cas9. Cell 162, 1113-1126, doi:10.1016/j.cell.2015.08.007 (2015).
43 Anzalone, A. V. et al. Search- and-replace genome editing without double-strand breaks or donor DNA. Nature, doi:10.1038/s41586-019-1711-4 (2019).
44 Nishida, K. et al. Targeted nucleotide editing using hybrid prokaryotic and vertebrate adaptive immune systems. Science 353, aaf8729, doi:10.1126/science.aaf8729 (2016).
45 Moscou, M. J. & Bogdanove, A. J. A simple cipher governs DNA recognition by TAL effectors. Science 326, 1501, doi:10.1126/science.1178817 (2009).
46 Boch, J. et al. Breaking the code of DNA binding specificity of TAL-type III effectors. Science 326, 1509-1512, doi:10.1126/science.1178811 (2009).
47 Miller, J. C. et al. A TALE nuclease architecture for efficient genome editing. Nat Biotechnol 29, 143-148, doi:10.1038/nbt.1755 (2011).
48 Reyon, D. et al. FLASH assembly of TALENs for high-throughput genome editing. Nature Biotechnology 30, 460-465, doi: 10.103 8/nbt.2170 (2012).
49 Zhang, F. et al. Efficient construction of sequence-specific TAL effectors for modulating mammalian transcription. Nat Biotechnol 29, 149-153, doi:10.1038/nbt.1775 (2011).
50 Yang, L. et al. Engineering and optimising deaminase fusions for genome editing. Nature Communications 7, 13330, doi:10.1038/ncomms13330 (2016).
51 Reyon, D. et al. Engineering customized TALE nucleases (TALENs) and TALE transcription factors by fast ligation-based automatable solid-phase high-throughput (FLASH) assembly. Curr Protoc Mol Biol Chapter 12, Unit 12 16, doi:10.1002/0471142727.mb1216s103 (2013).
52 Guilinger, J. P. et al. Broad specificity profiling of TALENs results in engineered nucleases with improved DNA-cleavage specificity. Nature Methods 11, 429-435, doi:10.1038/nmeth.2845 (2014).
53 Komor, A. C. et al. Improved base excision repair inhibition and bacteriophage Mu Gam protein yields C:G-to-T:A base editors with higher efficiency and product purity. Science Advances 3, eaao4774, doi:10.1126/sciadv.aao4774 (2017).
54 Koblan, L. W. et al. Improving cytidine and adenine base editors by expression optimization and ancestral reconstruction. Nature Biotechnology 36, 843, doi:10.1038/nbt.4172 nature.com/articles/nbt.4172 #supplementary-information (2018).
55 Lamb, B. M., Mercer, A. C. & Barbas, C. F., III. Directed evolution of the TALE N-terminal domain for recognition of all 5′ bases. Nucleic Acids Research 41, 9779-9785, doi:10.1093/nar/gkt754 (2013).
56 Nilsen, H. et al. Nuclear and mitochondrial uracil-DNA glycosylases are generated by alternative splicing and transcription from different positions in the UNG gene. Nucleic Acids Res 25, 750-755, doi:10.1093/nar/25.4.750 (1997).
57 Bharati, S., Krokan, H. E., Kristiansen, L., Otterlei, M. & Slupphaug, G. Human mitochondrial uracil-DNA glycosylase preform (UNG1) is processed to two forms one of which is resistant to inhibition by AP sites. Nucleic Acids Research 26, 4953-4959, doi:10.1093/nar/26.21.4953 (1998).
58 Lauritzen, K. H. et al. Mitochondrial DNA toxicity in forebrain neurons causes apoptosis, neurodegeneration, and impaired behavior. Mol Cell Biol 30, 1357-1367, doi:10.1128/MCB.01149-09 (2010).
59 Stuart, J. A. et al. DNA base excision repair activities and pathway function in mitochondrial and cellular lysates from cells lacking mitochondrial DNA. Nucleic acids research 32, 2181-2192, doi:10.1093/nar/gkh533 (2004).
60 Alexeyev, M. F. et al. Selective elimination of mutant mitochondrial genomes as therapeutic strategy for the treatment of NARP and MILS syndromes. Gene Therapy 15, 516-523, doi:10.1038/gt.2008.11 (2008).
61 Minczuk, M., Papworth, M. A., Miller, J. C., Murphy, M. P. & Klug, A. Development of a single-chain, quasi-dimeric zinc-finger nuclease for the selective degradation of mutated human mitochondrial DNA. Nucleic Acids Res 36, 3926-3938, doi:10.1093/nar/gkn313 (2008).
62 Andreazza, S. et al. Mitochondrially-targeted APOBEC1 is a potent mtDNA mutator affecting mitochondrial function and organismal fitness in Drosophila. Nat Commun 10, 3280, doi:10.1038/s41467-019-10857-y (2019).
63 Shokolenko, I., Venediktova, N., Bochkareva, A., Wilson, G. L. & Alexeyev, M. F. Oxidative stress induces degradation of mitochondrial DNA. Nucleic Acids Res 37, 2539-2548, doi:10.1093/nar/gkp100 (2009).
64 Liu, P. & Demple, B. DNA repair in mammalian mitochondria: Much more than we thought? Environ Mol Mutagen 51, 417-426, doi:10.1002/em.20576 (2010).
65 Gopal, R. K. et al. Early loss of mitochondrial complex I and rewiring of glutathione metabolism in renal oncocytoma. Proc Natl Acad Sci USA 115, E6283-E6290, doi:10.1073/pnas.1711888115 (2018).
66 Zhang, X. H., Tee, L. Y., Wang, X. G., Huang, Q. S. & Yang, S. H. Off-target Effects in CRISPR/Cas9-mediated Genome Engineering. Mol Ther Nucleic Acids 4, e264, doi:10.1038/mtna.2015.37 (2015).
67 Ludwig, L. S. et al. Lineage Tracing in Humans Enabled by Mitochondrial Mutations and Single-Cell Genomics. Cell 176, 1325-1339 e1322, doi:10.1016/j.cell.2019.01.022 (2019).
68 Corces, M. R. et al. An improved ATAC-seq protocol reduces background and enables interrogation of frozen tissues. Nat Methods 14, 959-962, doi:10.1038/nmeth.4396 (2017).
69 Xu, J. et al. Single-cell lineage tracing by endogenous mutations enriched in transposase accessible mitochondrial DNA. Elife 8, doi:10.7554/eLife.45105 (2019).
70 Cuculis, L., Abil, Z., Zhao, H. & Schroeder, C. M. Direct observation of TALE protein dynamics reveals a two-state search mechanism. Nature Communications 6, 7277, doi:10.1038/ncomms8277 (2015).
71 Rinaldi, F. C., Doyle, L. A., Stoddard, B. L. & Bogdanove, A. J. The effect of increasing numbers of repeats on TAL effector DNA binding specificity. Nucleic Acids Res 45, 6960-6970, doi:10.1093/nar/gkx342 (2017).
72 Meckler, J. F. et al. Quantitative analysis of TALE-DNA interactions suggests polarity effects. Nucleic Acids Res 41, 4118-4128, doi:10.1093/nar/gkt085 (2013).
73 Rogers, J. M. et al. Context influences on TALE-DNA binding revealed by quantitative profiling. Nat Commun 6, 7440, doi:10.1038/ncomms8440 (2015).
74 Thuronyi, B. W. et al. Continuous evolution of base editors with expanded target compatibility and improved activity. Nat Biotechnol 37, 1070-1079, doi:10.1038/s41587-019-0193-0 (2019).
75 Naeem, M. M. & Sondheimer, N. in Mitochondria in Health and in Sickness (eds Andrea Urbani & Mohan Babu) 257-267 (Springer Singapore, 2019).
76 Filograna, R. et al. Modulation of mtDNA copy number ameliorates the pathological consequences of a heteroplasmic mtDNA mutation in the mouse. Sci Adv 5, eaav9824, doi:10.1126/sciadv.aav9824 (2019).
77 Heberle, H., Meirelles, G. V., da Silva, F. R., Telles, G. P. & Minghim, R. InteractiVenn: a web-based tool for the analysis of sets through Venn diagrams. BMC Bioinformatics 16, 169, doi:10.1186/s12859-015-0611-3 (2015).
78 Shi, K. et al. Structural basis for targeted DNA cytosine deamination and mutagenesis by APOBEC3A and APOBEC3B. Nat Struct Mol Biol 24, 131-139, doi:10.1038/nsmb.3344 (2017).
79 Rietsch, A., Vallet-Gely, I., Dove, S. L. & Mekalanos, J. J. ExsE, a secreted regulator of type III secretion genes in Pseudomonas aeruginosa. Proc Natl Acad Sci USA 102, 8006-8011, doi:10.1073/pnas.0503005102 (2005).
80 Fazli, M., Harrison, J. J., Gambino, M., Givskov, M. & Tolker-Nielsen, T. In-Frame and Unmarked Gene Deletions in Burkholderia cenocepacia via an Allelic Exchange System Compatible with Gateway Technology. Appl Environ Microbiol 81, 3623-3630, doi:10.1128/AEM.03909-14 (2015).
81 Choi, K. H., DeShazer, D. & Schweizer, H. P. mini-Tn7 insertion in bacteria with multiple glmS-linked attTn7 sites: example Burkholderia mallei ATCC 23344. Nat Protoc 1, 162-169, doi:10.1038/nprot.2006.25 (2006).
82 Rees, H. A., Wilson, C., Doman, J. L. & Liu, D. R. Analysis and minimization of cellular RNA editing by DNA adenine base editors. Sci Adv 5, eaax5717, doi:10.1126/sciadv.aax5717 (2019).
83 Otwinowski, Z. & Minor, W. Processing of X-ray diffraction data collected in oscillation mode. Methods Enzymol 276, 307-326 (1997).
84 Adams, P. D. et al. PHENIX: a comprehensive Python-based system for macromolecular structure solution. Acta Crystallogr D Biol Crystallogr 66, 213-221, doi:10.1107/S0907444909052925 (2010).
85 Emsley, P. & Cowtan, K. Coot: model-building tools for molecular graphics. Acta Crystallogr D Biol Crystallogr 60, 2126-2132, doi:10.1107/S0907444904019158 (2004).
86 Painter, J. & Merritt, E. A. Optimal description of a protein structure in terms of multiple groups undergoing TLS motion. Acta Crystallogr D Biol Crystallogr 62, 439-450, doi:10.1107/S0907444906005270 (2006).
87 Chen, V. B. et al. MolProbity: all-atom structure validation for macromolecular crystallography. Acta Crystallogr D Biol Crystallogr 66, 12-21, doi:10.1107/S0907444909042073 (2010).
88 Li, H. & Durbin, R. Fast and accurate short read alignment with Burrows-Wheeler transform. Bioinformatics 25, 1754-1760, doi:10.1093/bioinformatics/btp324 (2009).
89 Koboldt, D. C. et al. VarScan 2: somatic mutation and copy number alteration discovery in cancer by exome sequencing. Genome Res 22, 568-576, doi:10.1101/gr.129684.111 (2012).
90 Clement, K. et al. CRISPResso2 provides accurate and rapid genome editing sequence analysis. Nat Biotechnol 37, 224-226, doi:10.1038/s41587-019-0032-3 (2019).
91 Doman, J. L. R., A., Newby, G. A. & Liu, D. R. Evaluation and minimization of Cas9-independent off-target DNA editing by cytosine base editors Nature Biotechnology in press (2019).

### Supplementary Discussion

### Toxicity of Intact DddAₜₒₓ Fused to DNA-Binding Proteins

To characterize DddAₜₒₓ activity in human cells, we transfected HEK293T cells with plasmids encoding DddAₜₒₓ fused to catalytically inactive S.pyogenes Cas9 (dSpCas9)^{1,2} or SpCas9(D10 Å) nickase^{1,3} (FIG. 40A). We observed a 2-10 fold lower viability in cells transfected with DddAₜₒₓ-Cas9 fusions compared to BE2 (a non-nicking cytosine base editor with two copies of UGI protein)⁴ and BE4max (an optimized current-generation cytosine base editor)⁵′⁶(FIG. 40B). While BE2 and BE4max exhibited between 5-17% and 17-55% target C·G-to-T·A conversion efficiency, respectively, (FIG. 40C), the widespread toxicity induced by DddAₜₒₓ-Cas9 precluded PCR amplification of target DNA sites from the few surviving cells for high-throughput sequencing.

### Editing Efficiencies of DddAₜₒₓ Splits G1322, A1343, N1357, G1371 and N1387

Splits A1343, G1371, and N1387 resulted in <0.1% conversion of C·G-to-T·A at TC target bases across all tested spacing lengths (FIGS. 32A-32H). G1322 and N1357 gave a maximum of 8.3% and 22% C·G-to-T·A conversion, respectively, within a 44-bp spacing regions (FIG. 32G), but editing efficiencies at other lengths of spacing regions were generally below 5.6% (FIGS. 32A-32H).

Indel Frequencies Associated with Split-DddAₜₒₓ-Cas9 Fusions

The level of indels associated with splits that afforded the highest editing efficiency (G1333 and G1397) were higher than those of canonical cytosine base editors⁶, up to 46% for some split fusion combinations (FIG. 43). We speculate that strand nicking by SaKKH-Cas9(D10 Å), combined with uracil excision on either strand⁷ can induce double-strand breaks that induce indel formation through end-joining processes^{8,9}.

### Preliminary Observations Governing Editing Efficiency and Selectivity of Split-DddAₜₒₓ for Subsequent DdCBE Editing

Cytidines in TCC contexts appeared to be favorable substrates for deamination for split-DddAₜₒₓ. TCC target bases are generally deaminated at varying efficiencies depending on their positions within the dsDNA spacing region (FIGS. 32D-32H). We observed modest C·G-to-T·A conversions between 2.0-8.5% within spacing region lengths of 28-, 33- and 39-bp (FIGS. 32D-32F). Editing efficiencies were elevated to 48±1.7% and 26±0.65% at 44- and 60-bp spacing lengths, respectively (FIGS. 32G-32H). In addition to TCC, split-DddAₜₒₓ also mediated deamination of cytidines in TCA (FIGS. 30A-C, and FIGS. 32E-G) and TCT (FIGS. 32B-32C, and FIG. 32G) contexts.

The selectivity of editing also depended on the fusion orientation. For 17-bp spacing length, we observed preferential deamination of target TCs that were closer to the protospacer of the Cas9 monomer fused to DddAₜₒₓ-C. (FIG. 32D). For longer spacing region lengths (44- and 60-bp), target bases closer to the protospacer of the Cas9 monomer fused to DddAₜₒₓ-N were preferentially deaminated (FIGS. 32G-32H).

In light of the above results, we designed subsequent mitoTALE binding sites to flank a spacing region approximately 15-18 bp long that contains TCC and/or TCA target bases. To assess the substrate selectivity and editing efficiency of mitoTALE-split-DddAₜₒₓ, we also included the two possible orientations for each G1333 and G1397 split.

### Efforts to Increase Mitochondrial Localization of MTS-mitoTALE-Split-DddAₜₒₓ-1×UGI

We fused zmLOC100282174, a Zea mays-derived MTS previously reported to induce high mitochondrial localization in human lymphoblasts¹⁰, in tandem to SOD2 or COX8 Å MTS sequence. However, the dual MTS sequence lowered or had no effect on base editing efficiencies (FIG. 44), suggesting that these MTS variants may not be fully compatible with our constructs, or that mitochondrial localization may not be limiting the editing efficiency of these fusions.

Detecting mtDNA Deletions in DdCBE-Treated Human HEK293T Cells

Long-range PCR of the whole mitochondrial genome resulted in a shorter DNA band for ND6-DdCBE-treated cells compared to amplicons obtained from cells treated with ND6-dead-DdCBE (FIG. 36B). The truncated amplicon from positions 2478-10858 of the mitochondrial DNA restored to its full length after day 12 (left panel), while the truncated amplicon from positions 2688-10653 persisted to the end of the experiment (day 18). However, we did not detect any large deletions in HEK293T cells 3 days after treatment with ND6-DdCBE (FIG. 47) which would have manifested as regions in the mtDNA with no sequencing coverage, suggesting that shorter amplicons observed in the DNA gel may arise from PCR artefacts.

### DURABILITY OF C·G-TO-T·A MITOCHONDRIAL DNA CONVERSIONS IN HEK293T CELLS

From day 12 to day 18, cells treated with ND6-DdCBE had a 6.2% decrease out of 27-34% total edits at C6 and C₇ (FIG. 37A). Missense mutations induced by DdCBEs could have impaired mitochondrial fitness, thereby placing them at a selective disadvantage for mtDNA replication. We also noted that BE4max-mediated editing of C₅ and C6, which decreased by 12% out of 57-70% total editing (FIG. 37E).

### Three-Day Time Course Expression Studies on DdCBEs

We wondered if DdCBE expression levels could explain the differences in average off-target editing frequencies and SNV numbers among standard DdCBEs that share the same architectures of wild-type NTDs and deaminases, but contain different lengths and sequences of TALE array repeats. We compared expression levels of ND5.1-DdCBE, ND5.2-DdCBE and ATP8-DdCBE over a three-day period and observed no significant differences, suggesting that the steady-state expression of DdCBEs cannot account for the differences in off-target activities (FIGS. 48A-48B and FIG. 53).

Predicted Effects of Off-Target SNVs on mtDNA Sequence and Protein Function

Approximately one-third of the total off-target mutations for each DdCBE resulted in missense mutations, while approximately two-thirds of the off-target mutations were in non-coding regions or led to synonymous mutations in coding regions (FIG. 49A). Among the missense mutations, more than half were predicted to be deleterious to protein function (FIG. 49B). We note that unannotated SNVs in non-coding rRNA and tRNA, which were excluded in SIFT analysis, could also have functional consequences”. To validate the SNV-calling pipeline, we selected 1-18 SNVs called for the different DdCBEs for targeted amplicon sequencing and verified all 35 to be bona-fide off-target SNVs (FIG. 49C).

### REFERENCES FOR SUPPLEMENTARY DISCUSSION

1 Jinek, M. et al. A programmable dual-RNA-guided DNA endonuclease in adaptive bacterial immunity. Science 337, 816-821, doi:10.1126/science.1225829 (2012).
2 Qi, L. S. et al. Repurposing CRISPR as an RNA-guided platform for sequence-specific control of gene expression. Cell 152, 1173-1183, doi:10.1016/j.cell.2013.02.022 (2013).
3 Cong, L. et al. Multiplex Genome Engineering Using CRISPR/Cas Systems. Science 339, 819-823, doi:10.1126/science.1231143 (2013).
4 Komor, A. C., Kim, Y. B., Packer, M. S., Zuris, J. A. & Liu, D. R. Programmable editing of a target base in genomic DNA without double-stranded DNA cleavage. Nature 533, 420, doi:10.1038/nature17946 nature.com/articles/nature17946 #supplementary-information (2016).
5 Komor, A. C. et al. Improved base excision repair inhibition and bacteriophage Mu Gam protein yields C:G-to-T:A base editors with higher efficiency and product purity. Science Advances 3, eaao4774, doi:10.1126/sciadv.aao4774 (2017).
6 Koblan, L. W. et al. Improving cytidine and adenine base editors by expression optimization and ancestral reconstruction. Nature Biotechnology 36, 843, doi:10.1038/nbt.4172 nature.com/articles/nbt.4172 #supplementary-information (2018).
7 Krokan, H. E. & Bjsris, M. Base Excision Repair. Cold Spring Harbor Perspectives in Biology 5, doi:10.1101/cshperspect.a012583 (2013).
8 Bothmer, A. et al. Characterization of the interplay between DNA repair and CRISPR/Cas9-induced DNA lesions at an endogenous locus. Nature Communications 8, 13905, doi:10.1038/ncomms13905 (2017).
9 Harrison, L., Brame, K. L., Geltz, L. E. & Landry, A. M. Closely opposed apurinic/apyrimidinic sites are converted to double strand breaks in Escherichia coli even in the absence of exonuclease III, endonuclease IV, nucleotide excision repair and AP lyase cleavage. DNA Repair (Amst) 5, 324-335, doi:10.1016/j.dnarep.2005.10.009 (2006).
10 Chin, R. M., Panavas, T., Brown, J. M. & Johnson, K. K. Optimized Mitochondrial Targeting of Proteins Encoded by Modified mRNAs Rescues Cells Harboring Mutations in mtATP6. Cell Rep 22, 2818-2826, doi:10.1016/j.celrep.2018.02.059 (2018).
11 Ruiz-Pesini, E. et al. An enhanced MITOMAP with a global mtDNA mutational phylogeny. Nucleic Acids Res 35, D823-828, doi:10.1093/nar/gkl927 (2007).
12 Lott, M. T. et al. mtDNA Variation and Analysis Using Mitomap and Mitomaster. Curr Protoc Bioinformatics 44, 1 23 21-26, doi:10.1002/0471250953.bi0123s44 (2013).
13 Lin, Y. et al. SAPTA: a new design tool for improving TALE nuclease activity. Nucleic Acids Res 42, e47, doi:10.1093/nar/gkt1363 (2014).

### Tables

TABLE 2
Diffraction data collection and refinement
statistics for DddA/DddIA

| | Ddd_{A}/DddI_{A} SAD peak^{a} | |
|---|---|---|
| PDB accession code | 61308 | |
| Data Collection | | |
| Space group | P2₁2₁2 | |
| Cell dimension | | |
| a, b, c (Å) | 126.8, 145.0, 64.2 | |
| α, β, γ(°) | 90, 90, 90 | |
| Wavelength (Å) | 0.9790 | |
| Resolution (Å) | 38.6-2.5 | (2.53-2.50)^{b} |
| No. unique reflections | 41658 | (1339) |
| R_{merge} | 0.12 | (1.2) |
| I/σI | 26.7 | (2.0) |
| Completeness (%) | | |
| Total | 98.9 | (96.7) |
| Anomalous | 99.0 | |
| Redundancy | 13.0 | (12.6) |
| Wilson B-factor (Å²) | 46.2 | |
| Refinement | | |
| Resolution (Å) | 38.6-2.5 | (2.53-2.50) |
| No. reflections | 41655 | (2789) |
| Rwork Rfree (%) | 17.2/22.8 | (26.3/34.6) |
| No. atoms | 7889 | |
| Protein | 7796 | |
| Ligand/ion | 4 | |
| Water | 89 | |
| B-factors (Å²) | | |
| Protein | 59.2 | |
| Ligand/ion | 72.3 | |
| Water | 50.7 | |
| rmsd | | |
| Bond lengths (Å) | 0.012 | |
| Bond angles (°) | 1.091 | |
| Missing residues | | |

| | |
|---|---|
| chain A | 1250-1289, 1423-1427 |
| chain B | 71-3 |
| chain C | 1250-1289, 1423-1427 |
| chain D | 72-3 |
| chain E | 1250-1290, 1423-1427 |
| chain F | none |
| chain G | 1250-1290, 1423-1427 |
| chain H | none |

TABLE 3
MitoTALE binding sites for each DdCBE.
Sequences start from the 5′ nucleotide recognized by the first repeat in the TALE array
(N₁ position) and end with the nucleotide recognized by the carboxy-terminal truncated
‘half’ repeat. De novo TALE-binding sequences were designed using the following
guidelines¹³: (i) 15-20 bp long, (ii) thymidine at the 3′-end, (iii) large %C within the
first 5 nucleotides if possible and (iv) large %T within the last 5 nucleotides if possible.
Remaining target sequences were from previously published mitoTALEs (green) or adapted to
recognize a different nucleotide at the N₁ position (red). See the Supplementary Sequences
section below for mitoTALE sequences. Top to bottom the left-mitoTALE sequences correspond
to SEQ ID NO: 146-152. Top to bottom the right-mitoTALE sequences correspond to SEQ ID
NO: 153-159.

| | Left-mito TALE | Right-mitoTALE |
|---|---|---|
| DdCBE | target sequence | target sequence |
| ND1 | 5′-CTAGCCTAGCCGTTT-3′ | 5′-GAGTTTGATGCTCACCCT-3′ |
| | De novo | De novo |
| ND2 | 5′-CTTAGCATACTCCTCAAT-3′ | 5′-AGAACTGCTATTATT-3′ |
| | De novo | De novo |
| ND4 | 5′-GCTAGTAACCACGTTCT-3′ | 5′-CCTGTAAGTAGGAGAGT-3′ |
| | De novo | De novo |
| ND5.1 | 5′-AGCATTAGCAGGAAT-3′ | 5′-CTTTGGAGTAGAA-3′ |
| | From Hashimoto. M et al, 2015 | Adapted from Hashimoto. M et al., 2015 |
| | | to recognize C over T at the N, position |
| ND5.2 | 5′-CAAAACCATACCTCT-3′ | 5′-GCTAGGCTGCCAATGT-3′ |
| | De novo | From Bacman. S et al, 2013 |
| ND5.3 | 5′-CAGTTCTTCAAATAT-3′ | AAGATTAGTATGGT |
| | De novo | Ds novo |
| ATP8 | 5′-ATTAAACACAAACTAT-3′ | 5′-ATGGGCTTTGGT-3′ |
| | From Bacman. S et al, 2013 | De novo |

TABLE 4
P-values from comparison of editing efficiencies from time course experiments in
HEK293T cells and U2OS cells.
For a given DdCBE, the P-value for editing efficiencies of target cytidine across two
cumulative timepoints is shown. P-values were calculated using the Student's two-tailed paired
t-test. Entries are highlighted in red if the P-value indicated a significant difference (P < 0.05).
HEK293T cells

| Base editor and | Day3 VS | Day6 VS | Day12 VS |
|---|---|---|---|
| cytidine position | Day6 | Day12 | Day18 |
| ND6-DdCBE, C6 | 0.003248338 | 0.367579396 | 0.015940359 |
| ND6-DdCBE, C7 | 0.002586263 | 0.565703966 | 0.020813553 |
| ND6-DdCBE, C11 | 0.007464037 | 0.3659328 | 0.013874452 |
| ND6-DdCBE, C13 | 0.002564148 | 0.45334421 | 0.014317529 |
| ND5.1-DdCBE, C10 | 0.004716876 | 0.094419717 | 0.047243751 |
| ND5.2-DdCBE, C12 | 0.023166261 | 0.064203104 | 0.058523608 |
| ND5.2-DdCBE, C13 | 0.028990618 | 0.025844251 | 0.052962149 |
| ATP8-DdCBE, C12 | 0.004113271 | 0.048822963 | 0.052625302 |
| ATP8-DdCBE, C13 | 0.011630525 | 0.099114459 | 0.042920243 |
| BE2, C5 | 0.732951948 | 0.035726897 | 0.00268113 |
| BE2, C6 | 0.746509043 | 0.057803697 | 0.005118562 |
| BE4max, CS | 0.630498196 | 0.838864469 | 0.010147598 |
| BE4max, C6 | 0.575297544 | 0.865037977 | 0.008872013 |

U2OS cells

| Base editor and | Day3 VS | Day6 VS | Day12 VS |
|---|---|---|---|
| cytidine position | Day6 | Day12 | Day18 |
| ND6-DdCBE, C6 | 0.000590938 | 0.390452684 | 0.069490317 |
| ND6-DdCBE, C7 | 0.000492063 | 0.464334118 | 0.146219197 |
| ND6-DdCBE, C11 | 0.007736858 | 0.191482179 | 0.042318087 |
| ND6-DdCBE, C13 | 0.003832983 | 0.194483146 | 0.074581243 |
| ND5.1-DdCBE, C10 | 0.003808745 | 0.328396021 | 0.189480814 |
| ND5.2-DdCBE, C12 | 0.13305717 | 0.937487147 | 0.484054682 |
| ND5.2-DdCBE, C13 | 0.190940372 | 0.54498276 | 0.48011839 |
| ATP8-DdCBE, C12 | 0.107997405 | 0.427059997 | 0.041850528 |
| ATP8-DdCBE, C13 | 0.172196805 | 0.381406262 | 0.030673134 |
| CCR5-DdCBE, C9 | 0.29867603 | 0.934543801 | 0.6557219 |
| CCR5-DdCBE, C10 | 0.299897207 | 0.904216914 | 0.56590558 |
| CCR5-DdCBE, C16 | 0.273229353 | 0.769787171 | 0.967771498 |

TABLE 5
Unique off-target SNVs mediated by DdCBEs.

| SN | Reference | Altered | Average |
|---|---|---|---|
| position | nucleotide | nucleotide | % frequency |
| 64 | C | T | 1.34% |
| 94 | G | A | 0.35% |
| 120 | C | T | 0.47% |
| 147 | C | T | 0.39% |
| 150 | C | T | 0.86% |
| 162 | C | T | 0.21% |
| 229 | G | A | 0.16% |
| 320 | C | T | 0.09% |
| 431 | C | T | 0.07% |
| 461 | C | T | 0.40% |
| 486 | C | T | 0.18% |
| 505 | C | T | 1.74% |
| 594 | C | T | 0.51% |
| 597 | C | T | 0.55% |
| 622 | G | A | 0.69% |
| 635 | C | T | 0.97% |
| 660 | C | T | 5.79% |
| 705 | C | T | 1.13% |
| 712 | C | T | 0.29% |
| 734 | C | T | 0.07% |
| 740 | G | A | 1.34% |
| 759 | C | T | 0.36% |
| 822 | G | A | 0.17% |
| 880 | C | T | 0.91% |
| 904 | C | T | 0.55% |
| 909 | G | A | 0.34% |
| 951 | G | A | 0.08% |
| 954 | C | T | 0.27% |
| 962 | C | T | 0.08% |
| 1007 | G | A | 0.07% |
| 1018 | G | A | 0.10% |
| 1074 | G | A | 0.59% |
| 1133 | C | T | 0.08% |
| 1170 | G | A | 0.82% |
| 1190 | C | T | 0.44% |
| 1216 | C | T | 0.24% |
| 1217 | G | A | 0.10% |
| 1227 | G | A | 1.69% |
| 1230 | C | T | 0.16% |
| 1236 | C | T | 0.27% |
| 1250 | C | T | 0.41% |
| 1271 | C | T | 0.43% |
| 1282 | G | A | 0.48% |
| 1290 | C | T | 0.07% |
| 1320 | G | A | 0.37% |
| 1330 | C | T | 0.47% |
| 1345 | G | A | 0.08% |
| 1411 | G | A | 0.09% |
| 1412 | G | A | 0.38% |
| 1414 | C | T | 0.55% |
| 1415 | G | A | 0.28% |
| 1422 | G | A | 0.35% |
| 1469 | G | A | 0.21% |
| 1491 | C | T | 1.61% |
| 1497 | C | T | 0.26% |
| 1500 | C | T | 0.37% |
| 1511 | C | T | 0.07% |
| 1516 | G | A | 0.49% |
| 1554 | G | A | 0.11% |
| 1561 | C | T | 0.08% |
| 1595 | G | A | 4.53% |
| 1660 | G | A | 0.11% |
| 1709 | G | A | 0.49% |
| 1750 | G | A | 0.35% |
| 1831 | G | A | 1.35% |
| 1913 | G | A | 2.41% |
| 1951 | C | T | 0.48% |
| 1970 | G | A | 0.64% |
| 1990 | G | A | 1.59% |
| 2024 | C | T | 2.23% |
| 2070 | C | T | 0.08% |
| 2076 | C | T | 0.11% |
| 2100 | C | T | 1.99% |
| 2121 | G | A | 0.56% |
| 2257 | C | T | 0.35% |
| 2269 | G | A | 2.45% |
| 2328 | C | T | 0.10% |
| 2331 | C | T | 0.87% |
| 2347 | C | T | 1.64% |
| 2349 | G | A | 0.07% |
| 2366 | G | A | 0.42% |
| 2423 | C | T | 0.51% |
| 2511 | C | T | 0.40% |
| 2523 | C | T | 0.50% |
| 2553 | G | A | 0.10% |
| 2611 | C | T | 0.16% |
| 2622 | G | A | 0.79% |
| 2643 | G | A | 0.08% |
| 2657 | C | T | 0.07% |
| 2692 | G | A | 0.47% |
| 2716 | G | A | 1.07% |
| 2719 | G | A | 0.07% |
| 2724 | G | A | 0.09% |
| 2779 | C | T | 0.75% |
| 2819 | G | A | 0.66% |
| 2824 | C | T | 0.47% |
| 2826 | G | A | 0.32% |
| 2842 | C | T | 0.14% |
| 2844 | G | A | 0.09% |
| 2865 | C | T | 0.10% |
| 2872 | C | T | 0.07% |
| 2899 | C | T | 1.09% |
| 2909 | G | A | 0.78% |
| 2917 | G | A | 0.50% |
| 2934 | G | A | 0.36% |
| 2948 | C | T | 0.24% |
| 2961 | C | T | 0.61% |
| 2983 | G | A | 0.50% |
| 2988 | C | T | 0.09% |
| 2989 | G | A | 1.08% |
| 2996 | G | A | 1.73% |
| 2999 | C | T | 0.27% |
| 3002 | G | A | 1.10% |
| 3007 | C | T | 2.38% |
| 3010 | G | A | 1.04% |
| 3047 | G | A | 0.31% |
| 3056 | C | T | 0.63% |
| 3063 | G | A | 0.08% |
| 3066 | C | T | 0.19% |
| 3075 | G | A | 0.17% |
| 3080 | G | A | 0.36% |
| 3087 | C | T | 0.97% |
| 3093 | C | T | 0.85% |
| 3119 | C | T | 0.47% |
| 3132 | G | A | 1.40% |
| 3155 | C | T | 0.09% |
| 3168 | C | T | 0.63% |
| 3187 | C | T | 0.09% |
| 3279 | C | T | 0.45% |
| 3324 | C | T | 0.25% |
| 3333 | C | T | 0.37% |
| 3351 | C | T | 0.51% |
| 3453 | C | T | 0.21% |
| 3457 | G | A | 1.53% |
| 3474 | C | T | 0.61% |
| 3503 | C | T | 0.46% |
| 3510 | C | T | 0.40% |
| 3522 | C | T | 1.13% |
| 3531 | G | A | 0.74% |
| 3549 | C | T | 0.57% |
| 3556 | C | T | 0.09% |
| 3573 | C | T | 0.16% |
| 3594 | C | T | 2.49% |
| 3600 | C | T | 0.13% |
| 3612 | C | T | 0.37% |
| 3654 | C | T | 0.37% |
| 3659 | G | A | 0.60% |
| 3662 | C | T | 0.76% |
| 3668 | G | A | 0.08% |
| 3693 | G | A | 0.49% |
| 3696 | C | T | 2.02% |
| 3738 | C | T | 0.20% |
| 3750 | C | T | 0.09% |
| 3804 | C | T | 0.21% |
| 3824 | G | A | 0.07% |
| 3839 | C | T | 0.26% |
| 3842 | G | A | 0.10% |
| 3869 | C | T | 0.26% |
| 3882 | G | A | 0.68% |
| 3900 | C | T | 0.46% |
| 3901 | G | A | 0.62% |
| 3903 | C | T | 0.08% |
| 3910 | G | A | 0.71% |
| 3920 | C | T | 1.20% |
| 3922 | G | A | 0.17% |
| 3930 | C | T | 0.17% |
| 3945 | C | T | 0.82% |
| 3966 | C | T | 0.63% |
| 3978 | C | T | 0.30% |
| 4032 | C | T | 0.52% |
| 4037 | G | A | 0.22% |
| 4048 | G | A | 1.02% |
| 4086 | C | T | 1.15% |
| 4127 | G | A | 0.08% |
| 4142 | G | A | 0.18% |
| 4153 | G | A | 1.98% |
| 4170 | C | T | 0.24% |
| 4185 | C | T | 0.32% |
| 4273 | C | T | 0.45% |
| 4275 | G | A | 0.18% |
| 4333 | G | A | 0.95% |
| 4345 | C | T | 0.08% |
| 4354 | C | T | 0.85% |
| 4364 | C | T | 0.40% |
| 4374 | C | T | 0.08% |
| 4387 | C | T | 0.24% |
| 4397 | C | T | 1.10% |
| 4410 | C | T | 0.59% |
| 4426 | C | T | 0.37% |
| 4461 | C | T | 0.42% |
| 4476 | C | T | 0.71% |
| 4493 | C | T | 1.40% |
| 4496 | C | T | 0.55% |
| 4526 | C | T | 0.63% |
| 4593 | C | T | 0.34% |
| 4620 | C | T | 0.08% |
| 4624 | C | T | 0.07% |
| 4629 | G | A | 0.09% |
| 4640 | C | T | 0.32% |
| 4652 | C | T | 0.60% |
| 4669 | C | T | 2.96% |
| 4676 | C | T | 6.03% |
| 4714 | G | A | 2.27% |
| 4789 | G | A | 0.41% |
| 4808 | C | T | 0.21% |
| 4814 | C | T | 1.60% |
| 4841 | G | A | 0.49% |
| 4846 | C | T | 4.69% |
| 4860 | C | T | 0.40% |
| 4867 | G | A | 0.43% |
| 4908 | C | T | 0.14% |
| 4912 | C | T | 0.07% |
| 4931 | C | T | 0.10% |
| 4934 | C | T | 0.79% |
| 4951 | C | T | 0.60% |
| 4955 | C | T | 0.17% |
| 4969 | G | A | 0.07% |
| 4975 | G | A | 1.31% |
| 5017 | C | T | 0.42% |
| 5032 | G | A | 0.87% |
| 5099 | C | T | 0.47% |
| 5147 | G | A | 0.18% |
| 5185 | G | A | 0.36% |
| 5202 | C | T | 0.32% |
| 5206 | C | T | 3.91% |
| 5207 | C | T | 0.07% |
| 5213 | C | T | 0.35% |
| 5216 | C | T | 0.30% |
| 5218 | C | T | 0.09% |
| 5224 | G | A | 0.26% |
| 5270 | C | T | 0.26% |
| 5271 | G | A | 0.60% |
| 5297 | C | T | 0.18% |
| 5300 | C | T | 1.09% |
| 5303 | C | T | 2.98% |
| 5312 | C | T | 0.33% |
| 5324 | C | T | 0.41% |
| 5330 | C | T | 0.10% |
| 5413 | G | A | 0.20% |
| 5444 | C | T | 0.08% |
| 5447 | C | T | 2.46% |
| 5448 | C | T | 0.39% |
| 5459 | C | T | 1.99% |
| 5532 | G | A | 0.44% |
| 5554 | C | T | 0.08% |
| 5591 | G | A | 0.67% |
| 5614 | C | T | 0.36% |
| 5660 | G | A | 0.36% |
| 5669 | G | A | 0.34% |
| 5790 | C | T | 0.78% |
| 5820 | C | T | 0.46% |
| 5822 | G | A | 0.07% |
| 5866 | C | T | 1.93% |
| 5875 | C | T | 0.20% |
| 5893 | C | T | 0.36% |
| 5903 | G | A | 0.70% |
| 5909 | C | T | 0.10% |
| 5913 | G | A | 1.46% |
| 5920 | G | A | 0.37% |
| 5950 | G | A | 0.23% |
| 5969 | C | T | 0.09% |
| 5987 | C | T | 1.00% |
| 6008 | C | T | 0.26% |
| 6015 | C | T | 0.11% |
| 6054 | G | A | 2.23% |
| 6062 | C | T | 0.18% |
| 6074 | C | T | 0.28% |
| 6077 | C | T | 2.66% |
| 6107 | C | T | 2.37% |
| 6122 | C | T | 0.21% |
| 6128 | C | T | 0.29% |
| 6130 | G | A | 0.35% |
| 6145 | G | A | 0.72% |
| 6164 | C | T | 0.49% |
| 6174 | G | A | 0.57% |
| 6211 | G | A | 0.20% |
| 6222 | C | T | 0.35% |
| 6242 | C | T | 0.14% |
| 6247 | C | T | 0.31% |
| 6258 | G | A | 0.39% |
| 6265 | G | A | 0.36% |
| 6271 | G | A | 0.36% |
| 6287 | C | T | 0.10% |
| 6294 | C | T | 0.12% |
| 6313 | C | T | 0.08% |
| 6322 | G | A | 0.07% |
| 6328 | C | T | 0.48% |
| 6347 | C | T | 0.07% |
| 6349 | C | T | 0.11% |
| 6368 | C | T | 0.43% |
| 6373 | C | T | 0.26% |
| 6398 | C | T | 0.51% |
| 6455 | C | T | 1.23% |
| 6458 | C | T | 2.26% |
| 6460 | G | A | 0.08% |
| 6463 | C | T | 1.96% |
| 6467 | C | T | 4.90% |
| 6473 | C | T | 0.33% |
| 6482 | C | T | 0.88% |
| 6506 | C | T | 1.07% |
| 6521 | C | T | 0.15% |
| 6537 | G | A | 0.19% |
| 6563 | C | T | 1.00% |
| 6564 | G | A | 0.71% |
| 6566 | C | T | 0.24% |
| 6573 | G | A | 0.40% |
| 6574 | G | A | 2.31% |
| 6577 | G | A | 1.18% |
| 6582 | G | A | 0.27% |
| 6621 | C | T | 0.58% |
| 6627 | G | A | 0.09% |
| 6644 | C | T | 0.65% |
| 6656 | C | T | 0.62% |
| 6658 | G | A | 0.08% |
| 6688 | C | T | 0.09% |
| 6725 | C | T | 0.35% |
| 6734 | G | A | 0.07% |
| 6749 | C | T | 0.57% |
| 6761 | C | T | 0.24% |
| 6790 | G | A | 0.08% |
| 6795 | G | A | 0.61% |
| 6801 | G | A | 0.10% |
| 6808 | G | A | 0.08% |
| 6823 | C | T | 0.52% |
| 6839 | C | T | 0.72% |
| 6845 | C | T | 0.42% |
| 6857 | C | T | 2.06% |
| 6871 | G | A | 0.38% |
| 6889 | G | A | 0.44% |
| 6907 | C | T | 0.09% |
| 6931 | G | A | 0.07% |
| 6962 | G | A | 0.43% |
| 6988 | C | T | 0.38% |
| 6993 | G | A | 0.09% |
| 6998 | C | T | 1.08% |
| 7008 | G | A | 0.38% |
| 7034 | C | T | 0.35% |
| 7043 | C | T | 0.90% |
| 7070 | C | T | 0.13% |
| 7075 | G | A | 0.33% |
| 7119 | G | A | 1.72% |
| 7139 | C | T | 0.10% |
| 7160 | C | T | 1.31% |
| 7181 | C | T | 1.48% |
| 7204 | C | T | 1.13% |
| 7207 | G | A | 0.19% |
| 7216 | G | A | 3.45% |
| 7225 | C | T | 0.41% |
| 7227 | G | A | 1.83% |
| 7236 | G | A | 1.14% |
| 7259 | C | T | 1.19% |
| 7267 | C | T | 0.17% |
| 7331 | C | T | 0.20% |
| 7336 | C | T | 0.51% |
| 7337 | G | A | 0.12% |
| 7349 | C | T | 0.37% |
| 7369 | C | T | 0.19% |
| 7384 | G | A | 0.44% |
| 7393 | G | A | 0.95% |
| 7419 | G | A | 0.65% |
| 7444 | G | A | 0.99% |
| 7454 | G | A | 0.45% |
| 7458 | G | A | 0.07% |
| 7502 | C | T | 0.62% |
| 7506 | G | A | 0.09% |
| 7550 | C | T | 0.56% |
| 7573 | C | T | 0.11% |
| 7616 | G | A | 0.43% |
| 7626 | C | T | 0.46% |
| 7637 | G | A | 0.18% |
| 7658 | G | A | 0.39% |
| 7661 | C | T | 0.44% |
| 7669 | C | T | 0.08% |
| 7693 | C | T | 0.52% |
| 7699 | C | T | 1.76% |
| 7754 | G | A | 0.52% |
| 7777 | C | T | 1.16% |
| 7779 | G | A | 0.08% |
| 7786 | C | T | 0.48% |
| 7798 | C | T | 0.40% |
| 7801 | C | T | 1.92% |
| 7807 | C | T | 1.75% |
| 7810 | C | T | 0.36% |
| 7813 | C | T | 1.41% |
| 7819 | C | T | 0.40% |
| 7824 | C | T | 0.97% |
| 7834 | C | T | 1.01% |
| 7847 | G | A | 1.17% |
| 7850 | G | A | 0.18% |
| 7855 | C | T | 0.49% |
| 7862 | C | T | 0.60% |
| 7866 | C | T | 0.35% |
| 7876 | C | T | 0.08% |
| 7919 | G | A | 1.30% |
| 7929 | G | A | 2.92% |
| 7939 | C | T | 0.10% |
| 7955 | C | T | 0.48% |
| 7979 | G | A | 0.40% |
| 7986 | G | A | 0.45% |
| 7994 | G | A | 1.03% |
| 8000 | G | A | 0.42% |
| 8020 | G | A | 0.23% |
| 8052 | C | T | 0.44% |
| 8057 | G | A | 0.30% |
| 8062 | C | T | 0.46% |
| 8080 | C | T | 0.79% |
| 8102 | G | A | 0.25% |
| 8115 | G | A | 5.88% |
| 8120 | C | T | 0.89% |
| 8148 | G | A | 1.16% |
| 8168 | C | T | 0.30% |
| 8187 | G | A | 0.28% |
| 8212 | C | T | 0.64% |
| 8215 | C | T | 6.92% |
| 8287 | C | T | 0.10% |
| 8428 | C | T | 0.07% |
| 8431 | C | T | 0.66% |
| 8434 | C | T | 1.14% |
| 8474 | C | T | 0.34% |
| 8478 | C | T | 0.38% |
| 8544 | C | T | 0.40% |
| 8549 | C | T | 0.09% |
| 8568 | C | T | 0.54% |
| 8592 | G | A | 0.21% |
| 8595 | C | T | 0.35% |
| 8605 | C | T | 0.09% |
| 8619 | C | T | 4.21% |
| 8620 | C | T | 0.11% |
| 8627 | C | T | 0.62% |
| 8635 | C | T | 0.34% |
| 8640 | C | T | 0.58% |
| 8648 | G | A | 1.05% |
| 8655 | C | T | 0.09% |
| 8669 | G | A | 0.31% |
| 8687 | C | T | 0.31% |
| 8720 | G | A | 11.41% |
| 8729 | G | A | 0.27% |
| 8747 | C | T | 0.08% |
| 8778 | C | T | 0.41% |
| 8781 | C | T | 0.91% |
| 8783 | G | A | 1.48% |
| 8844 | C | T | 1.76% |
| 8910 | C | T | 0.07% |
| 8940 | C | T | 1.00% |
| 8958 | C | T | 0.18% |
| 8967 | C | T | 0.19% |
| 9050 | G | A | 0.72% |
| 9085 | C | T | 0.50% |
| 9099 | C | T | 0.43% |
| 9102 | C | T | 0.08% |
| 9105 | C | T | 0.35% |
| 9123 | G | A | 0.30% |
| 9129 | C | T | 0.29% |
| 9144 | C | T | 1.17% |
| 9153 | C | T | 1.83% |
| 9196 | G | A | 1.19% |
| 9253 | G | A | 0.22% |
| 9281 | C | T | 1.03% |
| 9292 | C | T | 0.65% |
| 9317 | C | T | 0.63% |
| 9335 | C | T | 0.42% |
| 9376 | G | A | 0.17% |
| 9384 | G | A | 1.73% |
| 9394 | G | A | 0.08% |
| 9431 | C | T | 1.83% |
| 9444 | C | T | 0.30% |
| 9445 | G | A | 0.13% |
| 9452 | G | A | 0.34% |
| 9458 | C | T | 0.36% |
| 9472 | C | T | 0.47% |
| 9488 | C | T | 0.94% |
| 9544 | G | A | 0.09% |
| 9569 | C | T | 0.39% |
| 9582 | C | T | 0.54% |
| 9593 | C | T | 1.01% |
| 9610 | C | T | 1.93% |
| 9620 | C | T | 0.10% |
| 9625 | C | T | 0.78% |
| 9657 | C | T | 0.07% |
| 9731 | C | T | 0.17% |
| 9742 | C | T | 0.15% |
| 9752 | C | T | 0.34% |
| 9764 | C | T | 0.08% |
| 9772 | C | T | 0.07% |
| 9774 | G | A | 2.59% |
| 9782 | C | T | 0.09% |
| 9815 | C | T | 0.96% |
| 9820 | G | A | 0.79% |
| 9825 | C | T | 0.24% |
| 9830 | C | T | 0.77% |
| 9851 | C | T | 0.90% |
| 9866 | C | T | 1.77% |
| 9892 | C | T | 2.30% |
| 9900 | C | T | 0.34% |
| 9911 | C | T | 0.47% |
| 9912 | G | A | 0.26% |
| 9942 | G | A | 0.23% |
| 9952 | G | A | 0.27% |
| 9968 | C | T | 0.10% |
| 9974 | C | T | 0.20% |
| 9979 | G | A | 0.07% |
| 10022 | C | T | 0.27% |
| 10038 | G | A | 0.08% |
| 10067 | C | T | 0.26% |
| 10094 | C | T | 0.35% |
| 10126 | G | A | 0.08% |
| 10181 | C | T | 0.46% |
| 10182 | G | A | 0.46% |
| 10184 | C | T | 0.10% |
| 10192 | C | T | 1.17% |
| 10205 | C | T | 6.66% |
| 10213 | C | T | 0.20% |
| 10257 | C | T | 0.37% |
| 10271 | C | T | 0.08% |
| 10327 | C | T | 0.15% |
| 10330 | C | T | 0.88% |
| 10346 | C | T | 0.34% |
| 10349 | C | T | 1.36% |
| 10362 | C | T | 0.28% |
| 10375 | G | A | 0.31% |
| 10387 | G | A | 0.64% |
| 10437 | G | A | 0.36% |
| 10478 | C | T | 0.12% |
| 10518 | C | T | 0.07% |
| 10536 | C | T | 0.33% |
| 10552 | C | T | 0.70% |
| 10555 | C | T | 0.69% |
| 10573 | G | A | 0.07% |
| 10585 | C | T | 0.07% |
| 10616 | C | T | 0.07% |
| 10664 | C | T | 0.08% |
| 10677 | G | A | 0.34% |
| 10706 | C | T | 0.18% |
| 10731 | G | A | 0.30% |
| 10757 | C | T | 0.09% |
| 10774 | C | T | 0.18% |
| 10777 | C | T | 4.87% |
| 10801 | G | A | 0.16% |
| 10834 | C | T | 0.08% |
| 10867 | C | T | 0.10% |
| 10870 | C | T | 1.10% |
| 10917 | C | T | 0.46% |
| 10932 | C | T | 1.37% |
| 10933 | C | T | 0.08% |
| 10934 | G | A | 0.33% |
| 10954 | C | T | 0.44% |
| 10971 | G | A | 0.30% |
| 10984 | C | T | 0.07% |
| 11013 | C | T | 0.76% |
| 11125 | C | T | 0.82% |
| 11140 | C | T | 0.77% |
| 11158 | C | T | 0.46% |
| 11163 | G | A | 1.18% |
| 11166 | G | A | 0.29% |
| 11206 | C | T | 0.43% |
| 11234 | C | T | 0.41% |
| 11242 | C | T | 0.07% |
| 11245 | C | T | 0.80% |
| 11391 | G | A | 2.03% |
| 11422 | C | T | 4.55% |
| 11423 | G | A | 1.53% |
| 11434 | C | T | 0.58% |
| 11497 | C | T | 0.19% |
| 11518 | G | A | 1.20% |
| 11542 | C | T | 0.23% |
| 11592 | G | A | 1.44% |
| 11600 | G | A | 0.11% |
| 11628 | C | T | 0.10% |
| 11632 | C | T | 0.57% |
| 11647 | C | T | 2.09% |
| 11668 | C | T | 1.63% |
| 11679 | G | A | 0.17% |
| 11686 | C | T | 0.15% |
| 11698 | C | T | 0.24% |
| 11710 | C | T | 0.29% |
| 11718 | G | A | 0.67% |
| 11727 | C | T | 1.29% |
| 11730 | C | T | 0.33% |
| 11777 | C | T | 0.36% |
| 11782 | C | T | 0.20% |
| 11788 | C | T | 0.37% |
| 11799 | G | A | 0.34% |
| 11815 | C | T | 0.09% |
| 11840 | C | T | 0.21% |
| 11851 | C | T | 2.34% |
| 11860 | C | T | 1.82% |
| 11922 | G | A | 0.63% |
| 11925 | C | T | 0.36% |
| 11949 | G | A | 0.55% |
| 11965 | C | T | 0.37% |
| 12045 | C | T | 0.07% |
| 12054 | G | A | 0.25% |
| 12073 | C | T | 0.08% |
| 12084 | C | T | 0.45% |
| 12097 | C | T | 1.26% |
| 12102 | C | T | 0.64% |
| 12106 | C | T | 0.18% |
| 12113 | G | A | 0.83% |
| 12118 | C | T | 0.56% |
| 12162 | C | T | 0.56% |
| 12176 | G | A | 0.55% |
| 12192 | G | A | 0.47% |
| 12207 | G | A | 0.07% |
| 12246 | C | T | 0.28% |
| 12276 | G | A | 0.55% |
| 12288 | C | T | 0.42% |
| 12296 | C | T | 0.70% |
| 12377 | C | T | 1.39% |
| 12393 | C | T | 1.69% |
| 12405 | C | T | 0.30% |
| 12449 | C | T | 0.91% |
| 12456 | C | T | 2.06% |
| 12461 | C | T | 2.48% |
| 12474 | C | T | 0.32% |
| 12478 | C | T | 0.08% |
| 12483 | C | T | 1.15% |
| 12508 | G | A | 0.72% |
| 12583 | G | A | 0.65% |
| 12591 | C | T | 0.73% |
| 12593 | C | T | 0.46% |
| 12606 | C | T | 0.90% |
| 12621 | C | T | 0.44% |
| 12632 | C | T | 4.47% |
| 12636 | C | T | 0.32% |
| 12667 | G | A | 0.07% |
| 12682 | C | T | 0.37% |
| 12690 | C | T | 0.29% |
| 12708 | C | T | 0.60% |
| 12759 | C | T | 0.14% |
| 12762 | C | T | 1.29% |
| 12788 | C | T | 0.48% |
| 12806 | G | A | 0.10% |
| 12818 | G | A | 0.42% |
| 12823 | G | A | 0.20% |
| 12852 | C | T | 0.30% |
| 12867 | C | T | 0.46% |
| 12871 | G | A | 0.45% |
| 12876 | C | T | 0.54% |
| 12885 | C | T | 1.96% |
| 12888 | C | T | 0.71% |
| 12906 | C | T | 0.45% |
| 12925 | G | A | 0.30% |
| 12958 | C | T | 1.18% |
| 12966 | C | T | 0.09% |
| 12984 | C | T | 0.40% |
| 12987 | C | T | 0.43% |
| 13007 | C | T | 0.09% |
| 13021 | C | T | 0.17% |
| 13031 | G | A | 0.38% |
| 13040 | C | T | 0.36% |
| 13065 | C | T | 0.20% |
| 13119 | C | T | 2.25% |
| 13125 | C | T | 0.91% |
| 13155 | C | T | 0.71% |
| 13185 | C | T | 0.07% |
| 13197 | C | T | 0.23% |
| 13206 | C | T | 0.10% |
| 13230 | C | T | 0.43% |
| 13250 | C | T | 0.36% |
| 13261 | C | T | 0.08% |
| 13268 | G | A | 0.50% |
| 13287 | C | T | 0.26% |
| 13293 | C | T | 0.50% |
| 13323 | C | T | 0.42% |
| 13341 | C | T | 0.20% |
| 13364 | C | T | 0.19% |
| 13370 | C | 1 | 1.68% |
| 13374 | C | T | 0.29% |
| 13377 | C | T | 1.61% |
| 13393 | G | A | 0.18% |
| 13415 | G | A | 0.21% |
| 13418 | G | A | 0.86% |
| 13445 | C | T | 0.17% |
| 13451 | C | T | 0.19% |
| 13455 | C | T | 0.07% |
| 13481 | G | A | 0.07% |
| 13494 | C | T | 0.88% |
| 13508 | C | T | 0.18% |
| 13513 | G | A | 0.24% |
| 13521 | C | T | 0.80% |
| 13524 | C | T | 0.97% |
| 13578 | C | T | 0.90% |
| 13586 | C | T | 0.33% |
| 13590 | G | A | 0.70% |
| 13636 | C | T | 0.21% |
| 13642 | C | T | 0.24% |
| 13647 | C | T | 0.92% |
| 13715 | G | A | 1.01% |
| 13754 | C | T | 0.25% |
| 13763 | C | T | 5.08% |
| 13764 | C | T | 0.07% |
| 13770 | C | T | 1.28% |
| 13782 | C | T | 0.83% |
| 13809 | C | T | 0.09% |
| 13815 | C | T | 0.16% |
| 13826 | G | A | 0.23% |
| 13843 | G | A | 0.07% |
| 13880 | C | T | 0.07% |
| 13947 | C | T | 0.16% |
| 13992 | C | T | 1.62% |
| 13996 | G | A | 0.47% |
| 14006 | G | A | 0.49% |
| 14057 | C | T | 0.39% |
| 14061 | C | T | 0.27% |
| 14064 | C | T | 0.79% |
| 14100 | C | T | 0.38% |
| 14115 | C | T | 1.22% |
| 14124 | C | T | 2.34% |
| 14142 | C | T | 0.09% |
| 14155 | C | T | 0.32% |
| 14160 | G | A | 0.37% |
| 14259 | G | A | 0.11% |
| 14262 | C | T | 0.30% |
| 14265 | C | T | 0.81% |
| 14279 | G | A | 0.54% |
| 14292 | C | T | 0.17% |
| 14309 | C | T | 0.99% |
| 14346 | C | T | 0.35% |
| 14372 | C | T | 0.40% |
| 14379 | C | T | 0.43% |
| 14383 | C | T | 0.34% |
| 14438* | G* | A* | 17.85%* |
| 14439* | G* | A* | 20.74%* |
| 14443* | C* | T* | 3.43%* |
| 14445* | C* | T* | 13.25%* |
| 14446 | C | T | 0.20% |
| 14448 | C | T | 0.09% |
| 14471 | C | T | 0.81% |
| 14485 | C | T | 0.21% |
| 14531 | C | T | 0.53% |
| 14560 | G | A | 2.54% |
| 14601 | G | A | 0.51% |
| 14612 | G | A | 0.10% |
| 14671 | C | T | 0.26% |
| 14686 | G | A | 2.60% |
| 14698 | G | A | 4.24% |
| 14720 | C | T | 0.52% |
| 14749 | G | A | 0.20% |
| 14796 | C | T | 0.18% |
| 14803 | C | T | 0.94% |
| 14804 | G | A | 1.80% |
| 14806 | C | T | 0.32% |
| 14809 | C | T | 3.13% |
| 14810 | C | T | 0.89% |
| 14820 | C | T | 4.50% |
| 14827 | C | T | 0.26% |
| 14829 | C | T | 0.30% |
| 14835 | G | A | 0.48% |
| 14845 | C | T | 0.19% |
| 14854 | C | T | 0.08% |
| 14869 | G | A | 0.18% |
| 14872 | C | T | 2.53% |
| 14875 | C | T | 1.47% |
| 14889 | G | A | 1.35% |
| 14896 | C | T | 0.08% |
| 14918 | G | A | 0.65% |
| 14925 | C | T | 0.11% |
| 14940 | C | T | 0.34% |
| 14944 | C | T | 0.33% |
| 14953 | C | T | 0.66% |
| 14960 | G | A | 0.56% |
| 14983 | C | T | 3.36% |
| 14993 | C | T | 0.32% |
| 15009 | C | T | 0.30% |
| 15031 | C | T | 1.39% |
| 15040 | C | T | 2.78% |
| 15045 | G | A | 0.11% |
| 15060 | G | A | 1.48% |
| 15091 | C | T | 1.34% |
| 15100 | C | T | 0.60% |
| 15103 | C | T | 1.37% |
| 15142 | C | T | 1.18% |
| 15145 | C | T | 0.85% |
| 15150 | G | A | 0.34% |
| 15198 | C | T | 0.85% |
| 15205 | C | T | 1.24% |
| 15221 | G | A | 0.07% |
| 15240 | G | A | 0.45% |
| 15257 | G | A | 0.07% |
| 15263 | C | T | 0.81% |
| 15271 | C | T | 0.07% |
| 15295 | C | T | 0.18% |
| 15298 | C | T | 0.16% |
| 15337 | C | T | 0.49% |
| 15357 | G | A | 1.63% |
| 15384 | C | T | 1.34% |
| 15385 | C | T | 0.34% |
| 15390 | C | T | 0.08% |
| 15392 | G | A | 0.20% |
| 15406 | C | T | 0.81% |
| 15428 | G | A | 0.10% |
| 15436 | C | T | 0.19% |
| 15451 | C | T | 0.96% |
| 15493 | C | T | 0.26% |
| 15500 | G | A | 0.31% |
| 15506 | G | A | 0.51% |
| 15542 | C | T | 0.40% |
| 15550 | C | T | 1.32% |
| 15574 | C | T | 0.07% |
| 15591 | G | A | 0.43% |
| 15594 | C | T | 0.84% |
| 15598 | C | T | 3.60% |
| 15612 | G | A | 0.31% |
| 15619 | C | T | 3.41% |
| 15636 | C | T | 0.45% |
| 15640 | C | T | 0.72% |
| 15643 | C | T | 0.21% |
| 15646 | C | T | 1.05% |
| 15664 | C | T | 0.53% |
| 15667 | C | T | 0.35% |
| 15675 | C | T | 6.13% |
| 15676 | C | T | 0.15% |
| 15737 | G | A | 0.09% |
| 15742 | C | T | 0.34% |
| 15745 | C | T | 0.24% |
| 15760 | C | T | 0.09% |
| 15762 | G | A | 0.65% |
| 15765 | G | A | 1.26% |
| 15793 | C | T | 0.07% |
| 15798 | G | A | 1.94% |
| 15810 | C | T | 0.75% |
| 15832 | C | T | 0.08% |
| 15838 | C | T | 0.33% |
| 15890 | C | T | 0.77% |
| 15928 | G | A | 0.09% |
| 15930 | G | A | 0.10% |
| 15950 | G | A | 1.83% |
| 15957 | C | T | 0.07% |
| 16036 | G | A | 0.27% |
| 16173 | C | T | 0.26% |
| 16179 | C | T | 0.29% |
| 16193 | C | T | 0.19% |
| 16239 | C | T | 0.07% |
| 16363 | C | T | 3.22% |
| 16370 | G | A | 0.09% |
| 16393 | C | T | 15.92% |
| 16394 | C | T | 2.80% |
| 16407 | C | T | 0.82% |
| 16410 | C | T | 0.63% |
| 16425 | C | T | 0.44% |
| 16449 | C | T | 0.70% |
| 16494 | C | T | 0.39% |
| 16496 | G | A | 0.41% |
| 16501 | C | T | 0.62% |
| 16563 | C | T | 0.59% |
| 64 | C | T | 0.10% |
| 505 | C | T | 0.60% |
| 660 | C | T | 1.09% |
| 705 | C | T | 0.12% |
| 740 | G | A | 0.09% |
| 1227 | G | A | 0.28% |
| 1491 | C | T | 0.38% |
| 1595 | G | A | 0.62% |
| 1709 | G | A | 0.30% |
| 1831 | G | A | 0.40% |
| 1913 | G | A | 0.33% |
| 1990 | G | A | 0.19% |
| 2024 | C | T | 0.36% |
| 2100 | C | T | 0.09% |
| 2269 | G | A | 0.69% |
| 2347 | C | T | 0.27% |
| 2523 | C | T | 0.27% |
| 2716 | G | A | 0.37% |
| 2899 | C | T | 0.10% |
| 2989 | G | A | 0.08% |
| 2996 | G | A | 0.31% |
| 3007 | C | T | 0.44% |
| 3132 | G | A | 0.17% |
| 3351 | C | T | 0.16% |
| 3457 | G | A | 0.55% |
| 3522 | C | T | 0.08% |
| 3594 | C | T | 0.28% |
| 3662 | C | T | 0.08% |
| 3696 | C | T | 0.41% |
| 3920 | C | T | 0.21% |
| 4048 | G | A | 0.19% |
| 4153 | G | A | 0.54% |
| 4333 | G | A | 0.17% |
| 4397 | C | T | 0.19% |
| 4493 | C | T | 0.08% |
| 4526 | C | T | 0.16% |
| 4669 | C | T | 0.30% |
| 4714 | G | A | 0.42% |
| 4846 | C | T | 0.96% |
| 4975 | G | A | 0.07% |
| 5032 | G | A | 0.07% |
| 5099 | C | T | 0.26% |
| 5206 | C | T | 0.31% |
| 5300 | C | T | 0.14% |
| 5303 | C | T | 2.28% |
| 5447 | C | T | 0.40% |
| 5459 | C | T | 0.38% |
| 5866 | C | I | 0.27% |
| 5875 | C | T | 0.07% |
| 5913 | G | A | 0.26% |
| 5987 | C | T | 0.23% |
| 6054 | G | A | 0.08% |
| 6077 | C | T | 0.53% |
| 6128 | C | T | 0.16% |
| 6398 | C | T | 0.19% |
| 6455 | C | T | 0.31% |
| 6458 | C | T | 0.27% |
| 6463 | C | T | 0.30% |
| 6467 | C | T | 0.76% |
| 6563 | C | T | 0.14% |
| 6574 | G | A | 0.37% |
| 6839 | C | T | 0.23% |
| 6857 | C | T | 0.18% |
| 6998 | C | T | 0.32% |
| 7119 | G | A | 0.28% |
| 7160 | C | T | 0.34% |
| 7181 | C | T | 0.51% |
| 7204 | C | T | 0.32% |
| 7216 | G | A | 0.73% |
| 7227 | G | A | 0.20% |
| 7236 | G | A | 0.07% |
| 7259 | C | T | 0.07% |
| 7336 | C | T | 0.25% |
| 7393 | G | A | 0.10% |
| 7699 | C | T | 0.21% |
| 7801 | C | T | 0.38% |
| 7807 | C | T | 0.37% |
| 7813 | C | T | 0.35% |
| 7824 | C | T | 0.19% |
| 7834 | C | T | 0.17% |
| 7847 | G | A | 0.27% |
| 7919 | G | A | 0.07% |
| 7929 | G | A | 0.39% |
| 7994 | G | A | 0.20% |
| 8115 | G | A | 1.20% |
| 8120 | C | T | 0.17% |
| 8212 | C | T | 0.10% |
| 8215 | C | T | 1.49% |
| 8619 | C | T | 0.89% |
| 8648 | G | A | 0.33% |
| 8720 | G | A | 1.29% |
| 8781 | C | T | 0.17% |
| 8783 | G | A | 0.15% |
| 8844 | C | T | 0.40% |
| 9144 | C | T | 0.09% |
| 9153 | C | T | 0.84% |
| 9384 | G | A | 0.24% |
| 9431 | C | T | 0.39% |
| 9488 | C | T | 0.31% |
| 9610 | C | T | 0.24% |
| 9774 | G | A | 0.50% |
| 9815 | C | T | 0.10% |
| 9830 | C | T | 0.08% |
| 9851 | C | T | 0.26% |
| 9866 | C | T | 0.08% |
| 9892 | C | T | 0.32% |
| 9942 | G | A | 0.07% |
| 9952 | G | A | 0.28% |
| 10192 | C | T | 0.29% |
| 10205 | C | T | 1.53% |
| 10330 | C | T | 0.11% |
| 10349 | C | T | 0.26% |
| 10387 | G | A | 0.11% |
| 10552 | C | T | 0.08% |
| 10555 | C | T | 0.08% |
| 10777 | C | T | 0.77% |
| 10932 | C | I | 0.26% |
| 11013 | C | T | 0.09% |
| 11140 | C | T | 0.15% |
| 11163 | G | A | 0.29% |
| 11518 | G | A | 0.20% |
| 11592 | G | A | 0.13% |
| 11668 | C | T | 0.35% |
| 11727 | C | T | 0.35% |
| 11799 | G | A | 1.05% |
| 11804 | C | T | 0.53% |
| 12084 | C | T | 0.07% |
| 12296 | C | T | 0.08% |
| 12393 | C | I | 0.16% |
| 12456 | C | T | 0.51% |
| 12461 | C | T | 0.44% |
| 12483 | C | T | 0.07% |
| 12606 | C | T | 0.14% |
| 12632 | C | T | 0.99% |
| 12762 | C | T | 0.17% |
| 12885 | C | T | 0.18% |
| 12888 | C | T | 0.08% |
| 13119 | C | T | 0.36% |
| 13370 | C | T | 0.40% |
| 13377 | C | T | 0.67% |
| 13445 | C | T | 0.08% |
| 13494* | C* | T* | 33.00%* |
| 13496 | C | T | 0.56% |
| 13578 | C | T | 0.31% |
| 13590 | G | A | 0.44% |
| 13647 | C | T | 0.37% |
| 13763 | C | T | 0.95% |
| 13770 | C | T | 0.09% |
| 13782 | C | T | 0.08% |
| 13992 | C | T | 0.33% |
| 14006 | G | A | 0.08% |
| 14124 | C | T | 0.48% |
| 14265 | C | T | 0.08% |
| 14383 | C | T | 0.08% |
| 14560 | G | A | 0.69% |
| 14686 | G | A | 0.51% |
| 14698 | G | A | 0.63% |
| 14803 | C | T | 0.20% |
| 14820 | C | T | 0.40% |
| 14872 | C | T | 0.35% |
| 14875 | C | I | 0.31% |
| 14983 | C | T | 0.64% |
| 15031 | C | T | 0.19% |
| 15040 | C | T | 1.67% |
| 15060 | G | A | 0.09% |
| 15091 | C | T | 0.36% |
| 15103 | C | T | 0.22% |
| 15142 | C | T | 0.21% |
| 15198 | C | T | 0.07% |
| 15205 | C | T | 0.16% |
| 15357 | G | A | 0.28% |
| 15550 | C | T | 0.28% |
| 15594 | C | T | 0.33% |
| 15598 | C | T | 0.80% |
| 15619 | C | T | 0.58% |
| 15646 | C | T | 0.21% |
| 15664 | C | T | 0.20% |
| 15765 | G | A | 0.18% |
| 15798 | G | A | 0.23% |
| 15810 | C | I | 0.16% |
| 15950 | G | A | 0.36% |
| 16363 | C | T | 0.61% |
| 16449 | C | T | 0.16% |
| 933 | G | A | 0.09% |
| 1227 | G | A | 0.07% |
| 1625 | A | G | 0.07% |
| 3457 | G | A | 0.16% |
| 3696 | C | T | 0.19% |
| 4846 | C | T | 0.30% |
| 6876 | G | A | 0.07% |
| 7160 | C | T | 0.08% |
| 7216 | G | A | 0.10% |
| 8115 | G | A | 0.20% |
| 8148 | G | A | 0.07% |
| 8215 | C | T | 0.07% |
| 8720 | G | A | 0.16% |
| 9343 | G | A | 0.07% |
| 9384 | G | A | 0.20% |
| 9565 | G | A | 0.14% |
| 9774 | G | A | 0.07% |
| 10205 | C | T | 0.10% |
| 12145 | T | C | 0.18% |
| 12632 | C | T | 0.09% |
| 12871 | G | A | 0.07% |
| 13451 | C | T | 8.44% |
| 13452 | C | T | 5.89% |
| 13763 | C | T | 0.42% |
| 14560 | G | A | 0.08% |
| 14588 | C | T | 0.24% |
| 15040 | C | T | 0.19% |
| 933 | G | A | 0.09% |
| 1227 | G | A | 0.07% |
| 1625 | A | G | 0.07% |
| 3457 | G | A | 0.16% |
| 3696 | C | T | 0.19% |
| 4846 | C | T | 0.30% |
| 6876 | G | A | 0.07% |
| 7160 | C | T | 0.08% |
| 7216 | G | A | 0.10% |
| 8115 | G | A | 0.20% |
| 8148 | G | A | 0.07% |
| 8215 | C | T | 0.07% |
| 8720 | G | A | 0.16% |
| 9343 | G | A | 0.07% |
| 9384 | G | A | 0.20% |
| 9565 | G | A | 0.14% |
| 9774 | G | A | 0.07% |
| 10205 | C | T | 0.10% |
| 12145 | T | C | 0.18% |
| 12632 | C | T | 0.09% |
| 12871 | G | A | 0.07% |
| 13451 | C | T | 8.44% |
| 13452 | C | T | 5.89% |
| 13763 | C | T | 0.42% |
| 14560 | G | A | 0.08% |
| 14588 | C | T | 0.24% |
| 15040 | C | T | 0.19% |
| 933 | G | A | 0.09% |
| 1227 | G | A | 0.07% |
| 1625 | A | G | 0.07% |
| 3457 | G | A | 0.16% |
| 3696 | C | T | 0.19% |
| 4846 | C | T | 0.30% |
| 6876 | G | A | 0.07% |
| 7160 | C | T | 0.08% |
| 7216 | G | A | 0.10% |
| 8115 | G | A | 0.20% |
| 8148 | G | A | 0.07% |
| 8215 | C | T | 0.07% |
| 8720 | G | A | 0.16% |
| 9343 | G | A | 0.07% |
| 9384 | G | A | 0.20% |
| 9565 | G | A | 0.14% |
| 9774 | G | A | 0.07% |
| 10205 | C | T | 0.10% |
| 12145 | T | C | 0.18% |
| 12632 | C | T | 0.09% |
| 12871 | G | A | 0.07% |
| 13451* | C* | T* | 8.44%* |
| 13452* | C* | T* | 5.89%* |
| 13763 | C | T | 0.42% |
| 14560 | G | A | 0.08% |
| 14588 | C | T | 0.24% |
| 15040 | C | T | 0.19% |
| 546 | A | C | 0.11% |
| 574 | A | C | 0.12% |
| 1623 | G | A | 0.24% |
| 8115 | G | A | 0.09% |
| 11922* | G* | A* | 26.41%* |
| 11925 | C | I | 0.08% |
| 12145 | T | C | 0.09% |
| 13111 | T | C | 0.08% |
| 550 | A | C | 0.21% |
| 660 | C | T | 0.20% |
| 933 | G | A | 0.08% |
| 1169 | G | A | 0.00% |
| 1227 | G | A | 0.09% |
| 1415 | G | A | 0.76% |
| 1422 | G | A | 0.14% |
| 2948 | C | T | 0.61% |
| 3594 | C | T | 0.28% |
| 3696 | C | T | 0.09% |
| 5532 | G | A | 0.34% |
| 6467 | C | T | 0.08% |
| 6621 | C | T | 0.09% |
| 6876 | G | A | 0.09% |
| 7216 | G | A | 0.40% |
| 8115 | G | A | 0.09% |
| 8215 | C | T | 0.42% |
| 8474* | C* | T* | 7.21%* |
| 8475* | C* | T* | 3.64%* |
| 8844 | C | T | 0.97% |
| 8886 | G | A | 0.08% |
| 9384 | G | A | 0.81% |
| 10205 | C | T | 0.16% |
| 12632 | C | T | 0.18% |
| 12682 | C | T | 0.16% |
| 13377 | C | T | 0.27% |
| 14588 | C | T | 0.18% |
| 15031 | C | T | 0.65% |
| 15040 | C | T | 0.08% |
| 15205 | C | T | 0.08% |
| 15950 | G | A | 0.97% |

SNVs called by VarScan 2 were considered high-confidence if the percentage frequency of a given SNV is >0.1% in one or more replicates. Shown are all the unique SNVs across three independent biological replicates. SNV positions for each DdCBE treatment (ND6-DdCBE, italics; ND5.1-DdCBE, italics, underline; ND5.2 DdCBE, bold; ND4-DdCBE, bold, underlined; ATP8-DdCBE, bold, italics) were from the NC_012920 reference genome. On-target SNVs are marked by asterisks.

TABLE 6
Overlapping off-target SNVs.
Shown are the list of overlapping off-target SNVs between ND6- and
ND5.1-DdCBE (italics), ND6-, ND5.1- and ATP8-DdCBE (bold) and ND6-,
ND5.1-, ND5.2- and ATP8-DdCBE (underlined). The average frequencies
for the respective DdCBEs are shown.

| | | Average % frequency | | |
|---|---|---|---|---|
| SNV position | ND6-DdCBE | ND5.1-DdCBE | ND5.2-DdCBE | ATP8-DdCBE |
| 64 | 1.34% | 0.10% | | |
| 505 | 1.74% | 0.60% | | |
| 705 | 1.13% | 0.12% | | |
| 740 | 1.34% | 0.09% | | |
| 1491 | 1.61% | 0.38% | | |
| 1595 | 4.53% | 0.62% | | |
| 1709 | 0.49% | 0.30% | | |
| 1831 | 1.35% | 0.40% | | |
| 1913 | 2.41% | 0.33% | | |
| 1990 | 1.59% | 0.19% | | |
| 2024 | 2.23% | 0.36% | | |
| 2100 | 1.99% | 0.09% | | |
| 2269 | 2.45% | 0.69% | | |
| 2347 | 1.64% | 0.27% | | |
| 2523 | 0.50% | 0.27% | | |
| 2716 | 1.07% | 0.37% | | |
| 2899 | 1.09% | 0.10% | | |
| 2989 | 1.08% | 0.08% | | |
| 2996 | 1.73% | 0.31% | | |
| 3007 | 2.38% | 0.44% | | |
| 3132 | 1.40% | 0.17% | | |
| 3351 | 0.51% | 0.16% | | |
| 3522 | 1.13% | 0.08% | | |
| 3662 | 0.76% | 0.08% | | |
| 3920 | 1.20% | 0.21% | | |
| 4048 | 1.02% | 0.19% | | |
| 4153 | 1.98% | 0.54% | | |
| 4333 | 0.95% | 0.17% | | |
| 4397 | 1.10% | 0.19% | | |
| 4493 | 1.40% | 0.08% | | |
| 4526 | 0.63% | 0.16% | | |
| 4669 | 2.96% | 0.30% | | |
| 4714 | 2.27% | 0.42% | | |
| 4975 | 1.31% | 0.07% | | |
| 5032 | 0.87% | 0.07% | | |
| 5099 | 0.47% | 0.26% | | |
| 5206 | 3.91% | 0.31% | | |
| 5300 | 1.09% | 0.14% | | |
| 5303 | 2.98% | 2.28% | | |
| 5447 | 2.46% | 0.40% | | |
| 5459 | 1.99% | 0.38% | | |
| 5866 | 1.93% | 0.27% | | |
| 5875 | 0.20% | 0.07% | | |
| 5913 | 1.46% | 0.26% | | |
| 5987 | 1.00% | 0.23% | | |
| 6054 | 2.23% | 0.08% | | |
| 6077 | 2.66% | 0.53% | | |
| 6128 | 0.29% | 0.16% | | |
| 6398 | 0.51% | 0.19% | | |
| 6455 | 1.23% | 0.31% | | |
| 6458 | 2.26% | 0.27% | | |
| 6463 | 1.96% | 0.30% | | |
| 6563 | 1.00% | 0.14% | | |
| 6574 | 2.31% | 0.37% | | |
| 6839 | 0.72% | 0.23% | | |
| 6857 | 2.06% | 0.18% | | |
| 6998 | 1.08% | 0.32% | | |
| 7119 | 1.72% | 0.28% | | |
| 7181 | 1.48% | 0.51% | | |
| 7204 | 1.13% | 0.32% | | |
| 7227 | 1.83% | 0.20% | | |
| 7236 | 1.14% | 0.07% | | |
| 7259 | 1.19% | 0.07% | | |
| 7336 | 0.51% | 0.25% | | |
| 7393 | 0.95% | 0.10% | | |
| 7699 | 1.76% | 0.21% | | |
| 7801 | 1.92% | 0.38% | | |
| 7807 | 1.75% | 0.37% | | |
| 7813 | 1.41% | 0.35% | | |
| 7824 | 0.97% | 0.19% | | |
| 7834 | 1.01% | 0.17% | | |
| 7847 | 1.17% | 0.27% | | |
| 7919 | 1.30% | 0.07% | | |
| 7929 | 2.92% | 0.39% | | |
| 7994 | 1.03% | 0.20% | | |
| 8120 | 0.89% | 0.17% | | |
| 8212 | 0.64% | 0.10% | | |
| 8619 | 4.21% | 0.89% | | |
| 8648 | 1.05% | 0.33% | | |
| 8781 | 0.91% | 0.17% | | |
| 8783 | 1.48% | 0.15% | | |
| 9144 | 1.17% | 0.09% | | |
| 9153 | 1.83% | 0.84% | | |
| 9431 | 1.83% | 0.39% | | |
| 9488 | 0.94% | 0.31% | | |
| 9610 | 1.93% | 0.24% | | |
| 9815 | 0.96% | 0.10% | | |
| 9830 | 0.77% | 0.08% | | |
| 9851 | 0.90% | 0.26% | | |
| 9866 | 1.77% | 0.08% | | |
| 9892 | 2.30% | 0.32% | | |
| 9942 | 0.23% | 0.07% | | |
| 9952 | 0.27% | 0.28% | | |
| 10192 | 1.17% | 0.29% | | |
| 10330 | 0.88% | 0.11% | | |
| 10349 | 1.36% | 0.26% | | |
| 10387 | 0.64% | 0.11% | | |
| 10552 | 0.70% | 0.08% | | |
| 10555 | 0.69% | 0.08% | | |
| 10777 | 4.87% | 0.77% | | |
| 10932 | 1.37% | 0.26% | | |
| 11013 | 0.76% | 0.09% | | |
| 11140 | 0.77% | 0.15% | | |
| 11163 | 1.18% | 0.29% | | |
| 11518 | 1.20% | 0.20% | | |
| 11592 | 1.44% | 0.13% | | |
| 11668 | 1.63% | 0.35% | | |
| 11727 | 1.29% | 0.35% | | |
| 11799 | 0.34% | 1.05% | | |
| 12084 | 0.45% | 0.07% | | |
| 12296 | 0.70% | 0.08% | | |
| 12393 | 1.69% | 0.16% | | |
| 12456 | 2.06% | 0.51% | | |
| 12461 | 2.48% | 0.44% | | |
| 12483 | 1.15% | 0.07% | | |
| 12606 | 0.90% | 0.14% | | |
| 12762 | 1.29% | 0.17% | | |
| 12885 | 1.96% | 0.18% | | |
| 12888 | 0.71% | 0.08% | | |
| 13119 | 2.25% | 0.36% | | |
| 13370 | 1.68% | 0.40% | | |
| 13445 | 0.17% | 0.08% | | |
| 13494 | 0.88% | 33.00% | | |
| 13578 | 0.90% | 0.31% | | |
| 13590 | 0.70% | 0.44% | | |
| 13647 | 0.92% | 0.37% | | |
| 13770 | 1.28% | 0.09% | | |
| 13782 | 0.83% | 0.08% | | |
| 13992 | 1.62% | 0.33% | | |
| 14006 | 0.49% | 0.08% | | |
| 14124 | 2.34% | 0.48% | | |
| 14265 | 0.81% | 0.08% | | |
| 14383 | 0.34% | 0.08% | | |
| 14686 | 2.60% | 0.51% | | |
| 14698 | 4.24% | 0.63% | | |
| 14803 | 0.94% | 0.20% | | |
| 14820 | 4.50% | 0.40% | | |
| 14872 | 2.53% | 0.35% | | |
| 14875 | 1.47% | 0.31% | | |
| 14983 | 3.36% | 0.64% | | |
| 15060 | 1.48% | 0.09% | | |
| 15091 | 1.34% | 0.36% | | |
| 15103 | 1.37% | 0.22% | | |
| 15142 | 1.18% | 0.21% | | |
| 15198 | 0.85% | 0.07% | | |
| 15357 | 1.63% | 0.28% | | |
| 15550 | 1.32% | 0.28% | | |
| 15594 | 0.84% | 0.33% | | |
| 15598 | 3.60% | 0.80% | | |
| 15619 | 3.41% | 0.58% | | |
| 15646 | 1.05% | 0.21% | | |
| 15664 | 0.53% | 0.20% | | |
| 15765 | 1.26% | 0.18% | | |
| 15798 | 1.94% | 0.23% | | |
| 15810 | 0.75% | 0.16% | | |
| 16363 | 3.22% | 0.61% | | |
| 16449 | 0.70% | 0.16% | | |
| 660 | 5.79% | 1.09% | | 0.20% |
| 3594 | 2.49% | 0.28% | | 0.28% |
| 6467 | 4.90% | 0.76% | | 0.08% |
| 8844 | 1.76% | 0.40% | | 0.97% |
| 13377 | 1.61% | 0.67% | | 0.27% |
| 15031 | 1.39% | 0.19% | | 0.65% |
| 15205 | 1.24% | 0.16% | | 0.08% |
| 15950 | 1.83% | 0.36% | | 0.97% |
| 1227 | 1.69% | 0.28% | 0.07% | 0.09% |
| 3696 | 2.02% | 0.41% | 0.19% | 0.09% |
| 7216 | 3.45% | 0.73% | 0.10% | 0.40% |
| 8215 | 6.92% | 1.49% | 0.07% | 0.42% |
| 9384 | 1.73% | 0.24% | 0.20% | 0.81% |
| 10205 | 6.66% | 1.53% | 0.10% | 0.16% |
| 12632 | 4.47% | 0.99% | 0.09% | 0.18% |
| 15040 | 2.78% | 1.67% | 0.19% | 0.08% |

TABLE 7
Disease-associated mitochondrial DNA point mutations.
Pathogenic mtDNA point mutations were obtained from the MITOMAP
database12 (accessed Dec. 10, 2019). Disease-associated mutations
in rRNA/tRNA and coding/non-coding regions were considered only
if they had been assigned ‘Cfrm’ statuses. Italics: SNPs that
are corrected by C•G-to-T•A conversions; Underlined: SNPs
that are corrected by A•T-to-G•C conversions; Bold, underlined:
SNPs that are corrected by A•T-to-C•G conversions; Bold,
italics: SNPs that are corrected by C•G-to-G•C conversions;
Italics, underlined: SNPs that are corrected by A•T-to-T•A conversions

| Allele SNP | Locus | Associated disease |
|---|---|---|
| T616C | MT-TF | Maternally inherited epilepsy/kidney disease |
| A1555G | MT-RNR1 | DEAF; autism spectrum intellectual disability; possibly |
| | | antiatherosclerotic |
| A1630G | MT-TV | MNGIE-like disease/MELAS |
| A3243G | MT-TL1 | MELAS/LS/DMDF/MIDD/SNHL/CPEO/MM/FSGS/ASD/ |
| | | Cardiac + multi-organ dysfunction |
| T3258C | MT-TL1 | MELAS/Myopathy |
| A3260G | MT-TL1 | MMC/MELAS |
| T3271C | MT-TL1 | MELAS/DM |
| A3280G | MT-TL1 | Myopathy |
| T3291C | MT-TL1 | MELAS/Myopathy/Deafness + Cognitive Impairment |
| A3302G | MT-TL1 | MM |
| A4300G | MT-TI | MICM |
| A5690G | MT-TN | CPEO + ptosis + proximal myopathy |
| T5728C | MT-TN | Multiorgan failure/myopathy |
| A7445G | MT-TS1 precursor | SNHL |
| A7445G | MT-CO1 | SNHL |
| T7510C | MT-TS1 | SNHL |
| T7511C | MT-TS1 | SNHL/Deafness |
| A8344G | MT-TK | MERRF; Other-LD/Depressive mood disorder/ |
| | | leukoencephalopathy/HiCM |
| T8356C | MT-TK | MERRF |
| T8528C | MT-ATP8/6 | Infantile cardiomyopathy |
| T8851C | MT-ATP6 | BSN/Leigh syndrome |
| T8993C | MT-ATP6 | NARP/Leigh Disease/MILS/other |
| T9035C | MT-ATP6 | Ataxia syndromes |
| A9155G | MT-ATP6 | MIDD, renal insufficiency |
| T9176C | MT-ATP6 | FBSN/Leigh Disease |
| T9185C | MT-ATP6 | Leigh Disease/Ataxia syndromes/NARP-like disease |
| T10010C | MT-TG | PEM |
| T10158C | MT-ND3 | Leigh Disease/MELAS |
| T10191C | MT-ND3 | Leigh Disease/Leigh-like Disease/ESOC |
| T10663C | MT-ND4L | LHON |
| T12706C | MT-ND5 | Leigh Disease |
| T13094C | MT-ND5 | Ataxia + PEO/MELAS, LD, LHON, myoclonus, fatigue |
| A13514G | MT-ND5 | Leigh Disease/MELAS/Ca2+ downregulation |
| T14484C | MT-ND6 | LHON |
| T14487C | MT-ND6 | Dystonia/Leigh Disease/ataxia/ptosis/epilepsy |
| A14495G | MT-ND6 | LHON |
| T14674C | MT-TE | Reversible COX deficiency myopathy |
| T14709C | MT-TE | MM + DMDF/Encephalomyopathy/ |
| | | Dementia + diabetes + ophthalmoplegia |
| T14849C | MT-CYB | EXIT/Septo-Optic Dysplasia |
| T14864C | MT-CYB | MELAS |
| A15579G | MT-CYB | Multisystem Disorder, EXIT |
| G583A | MT-TF | MELAS/MM & EXIT |
| C1494T | MT-RNR1 | DEAF |
| G1606A | MT-TV | AMDF |
| G1644A | MT-TV | LS/HCM/MELAS |
| C3256T | MT-TL1 | MELAS; possible atherosclerosis risk |
| C3303T | MT-TL1 | MMC |
| G3376A | MT-ND1 | LHON MELAS overlap |
| G3460A | MT-ND1 | LHON |
| G3635A | MT-ND1 | LHON |
| G3697A | MT-ND1 | MELAS/LS/LDYT/BSN |
| G3700A | MT-ND1 | LHON |
| G3733A | MT-ND1 | LHON |
| G3890A | MT-ND1 | Progressive Encephalomyopathy/LS/Optic Atrophy |
| G4298A | MT-TI | CPEO/MS |
| G4308A | MT-TI | CPEO |
| G4332A | MT-TQ | Encephalopathy/MELAS |
| G4450A | MT-TM | Myopathy/MELAS |
| G5521A | MT-TW | Mitochondrial myopathy |
| G5650A | MT-TA | Myopathy |
| G5703A | MT-TN | CPEO/MM |
| G7497A | MT-TS1 | MM/EXIT |
| G8340A | MT-TK | Myopathy/Exercise Intolerance/Eye disease + SNHL |
| G8363A | MT-TK | MICM + DEAF/MERRF/Autism/LS/Ataxia + Lipomas |
| G8969A | MT-ATP6 | Mitochondrial myopathy, lactic acidosis and |
| | | sideroblastic anemia (MLASA)/IgG nephropathy |
| G10197A | MT-ND3 | Leigh Disease/Dystonia/Stroke/LDYT |
| G11778A | MT-ND4 | LHON/Progressive Dystonia |
| G12147A | MT-TH | MERRF-MELAS/Encephalopathy |
| G12276A | MT-TL2 | CPEO |
| G12294A | MT-TL2 | CPEO/EXIT + Ophthalmoplegia |
| G12315A | MT-TL2 | CPEO/KSS/possible carotid atherosclerosis risk, trend |
| | | toward myocardial infarction risk |
| G12316A | MT-TL2 | CPEO |
| G13042A | MT-ND5 | Optic neuropathy/retinopathy/LD |
| G13051A | MT-ND5 | LHON |
| G13513A | MT-ND5 | Leigh Disease/MELAS/LHON-MELAS Overlap Syndrome/ |
| | | negative association w Carotid Atherosclerosis |
| G14459A | MT-ND6 | LDYT/Leigh Disease/dystonia/carotid atherosclerosis risk |
| G14710A | MT-TE | Encephalomyopathy + Retinopathy |
| C4171A | MT-ND1 | LHON/Leigh-like phenotype |
| C11777A | MT-ND4 | Leigh Disease |
| C12258A | MT-TS2 | DMDF/RP + SNHL |
| C14482A | MT-ND6 | LHON |
| C14482G | MT-ND6 | LHON |
| A3243T | MT-TL1 | MM/MELAS/SNHL/CPEQ |

### Tables 8A-8B List of Bacterial Strains and Plasmids Used in Example1

Table 8A is a list of bacterial strains used in this study.

| Bacterial species and strains | Genotype | Purpose | Source |
|---|---|---|---|
| Escherichia coli DH5α | F- φ80lacZΔM15 Δ(lacZYA- | General cloning | Thermo Fisher |
| | argF)U169 recA1 endA1 | | Scientific |
| | hsdR17(rK−, mK+) phoA supE44 λ- | | Cat#18258012 |
| | thi-1 gyrA96 relA1 | | |
| Escherichia coli DH5α:Dddl | F- φ80lacZΔM15 Δ(lacZYA- | Cloning for full-length | This study |
| | argF)U169 recA1 endA1 | DddAₜₒₓ fusions | |
| | hsdR17(rK−, mK+) phoA supE44 A- | | |
| | thi-1 gyrA96 relA1, with | | |
| | chromosomally integrated dddl | | |
| Escherichia coli Mach1 | str. W | General cloning | Thermo Fisher |
| | ΔrecA1398 endA1 fhuA ϕ80Δ(lac)M | (mammalian plasmids) | Scientific Cat# |
| | 15 Δ(lac)X74 hsdR(r_{K}⁻ m_{K}⁺) | | C862003 |
| Escherichia coli BL21 | F⁻ ompT hsdS_{B} (r_{B}⁻, m_{B}⁻) gal | Protein extraction | EMD Millipore |
| | dcm (DE3) | | Cat#69450 |
| Escherichia coli XK1502 | F− ΔlacU169 nalA | Protein expression | PMID: |
| | | | 16923902 |
| Burkholderia cenocepacia | Wild type | Amplification of | PMID: |
| H111 | | dddA-dddA_{I}, parental | 10713433 |
| | | strain for mutant | |
| | | construction | |
| Burkholderia cenocepacia | Δ I35_RS01770, attTn7::aacC1 | Growth competition | This study |
| H111 AicmF1 GentR | | assays | |
| Burkholderia cenocepacia | Δ I35_RS17395, attTn7::aacC1 | Growth competition | This study |
| H111 AicmF2 GentR | | assays | |
| Burkholderia cenocepacia | ΔI35_RS34180Δ I35_RS34175, | Growth competition | This study |
| H111 AdddAAdddAl GentR | attTn7::aacC1 | assays | |
| Burkholderia cenocepacia | I35_RS34180^{E1347A}, attTn7::aacC1 | Growth competition | This study |
| H111 dddAE1347A | | assays | |
| Pseudomonas aeruginosa | Wild type | Growth competition | PMID: |
| PAO1 | | assays | 10984043 |

Table 8B is a list of plasmids used in this study.

| Plasmids | Purpose | Source |
|---|---|---|
| pPSV39-CV | For inducible expression of proteins in E. coli | PMID: 23954347 |
| pScrhaB2-V | For inducible expression of proteins in E. coli | PMID: 15925406 |
| pBAD24 | Protein expression | PMID: 7608087 |
| pETDuet-1 | Protein expression | EMD Millipore |
| | | Cat#71146-3 |
| pDONRPEX18Gm-Scel-pheS | Allellic replacement in B. cenocepacia | PMID: 25795676 |
| pDAI-Scel-pheS | Allellic replacement in B. cenocepacia | PMID: 25795676 |
| pUC18T-mini-Tn7T-Gm | For the generation of gentamycin resistant B. | PMID: 15908923 |
| | cenocepacia | |
| pScrhaB2-V ::dddA | To express dddA | This study |
| pScrhaB2-V ::cdd | To express cdd | This study |
| pScrhaB2-V::APOBEC3G | To express APOBEC3G | This study |
| pScrhaB2-V::tadA | To express tadA | This study |
| pPSV39-CV::dddA_{I} | To express dddAl | This study |
| pDONRPEX18Gm-Scel-pheS::ΔicmF1 | To delete icmF1 from B. cenocepacia | This study |
| pDONRPEX18Gm-Scel-pheS::ΔicmF2 | To delete icmF2 from B. cenocepacia | This study |
| pDONRPEX18Gm-Scel- | To delete dddA and dddA_{I} from B. cenocepacia | This study |
| pheS::ΔdddAΔdddA_{I} | | |
| pDONRPEX18Gm-Scel- | To generate the dddAE^{1347A} catalytic mutant in B. | This study |
| pheS::dddA^{E1347A} | cenocepacia | |
| pBAD24::ung | To express ung in E. coli | This study |
| pETDuet-1 mcs1::dddA-his₆ | To co-express dddA-dddA_{I} | This study |
| mcs1::dddA_{I} | | |
| pTNS3 | Tn7 transposase expression | PMID: 18156318 |
| pRK2013 | Helper plasmid for plasmid mobilization | ATCC ® 37159 |
| pKD46 | Recombinase expression plasmid for A Red | PMID: 10829079 |
| | deletion system | |
| pKD4 | Template for ung deletion amplicon | PMID: 10829079 |
| pCMV | For mammalian expression of BE2, BE4max, | PMID: 27096365 |
| | split DddAtox and all DddAtox fusions | PMID: 24531420 |
| | | for CCR5 TALEs |

TABLE 9A
shows gRNA sequences for Cas9 screening

| gRNA | Sequence (5′-to-3′) | SEQ ID NO: |
|---|---|---|
| spG7 | CCACTGGGGCCTCAACACTC | 332 |
| spG6 | GAGGCCCCCAGAGCAGCCAC | 333 |
| saG1 | TCTGTGCCCCTCCCTCCCTGGC | 334 |
| saG2 | GCCCCTCCCTCCCTGGCCCAGGT | 335 |
| saG3 | CCCTCCCTGGCCCAGGTGAAGG | 336 |
| saG4 | GTGTGGTTCCAGAACCGGAGGA | 337 |

dsDNA spacing length (bp)

| | saG1 | saG2 | saG3 | saG4 |
|---|---|---|---|---|
| spG7 | 28 | 33 | 39 | 60 |
| spG6 | 12 | 17 | 23 | 44 |

TABLE 10A
KKH-Cas9 half with saG4 gRNA

| Batch | Nucleotide | G | G | C | C | C | C | T | A | A | C | C | C |
|---|---|---|---|---|---|---|---|---|---|---|---|---|---|
| G1333 DddAtox-N- | A | 0.19% | 0.03% | 0.00% | 0.00% | 0.00% | 0.00% | 0.00% | 99.92% | 99.68% | 0.00% | 0.00% | 0.01% |
| SaKKH-Cas9 with | | | | | | | | | | | | | |
| saG4 gRNA | | | | | | | | | | | | | |
| G1333 DddAtox-N- | T | 0.24% | 0.03% | 0.01% | 0.00% | 0.01% | 0.01% | 99.95% | 0.01% | 0.01% | 0.00% | 0.01% | 0.00% |
| SaKKH-Cas9 with | | | | | | | | | | | | | |
| saG4 gRNA | | | | | | | | | | | | | |
| G1333 DddAtox-N- | C | 0.13% | 0.03% | 99.28% | 99.98% | 99.97% | 99.97% | 0.03% | 0.05% | 0.00% | 99.98% | 99.98% | 99.79% |
| SaKKH-Cas9 with | | | | | | | | | | | | | |
| saG4 gRNA | | | | | | | | | | | | | |
| G1333 DddAtox-N- | G | 99.44% | 99.91% | 0.70% | 0.01% | 0.00% | 0.00% | 0.00% | 0.02% | 0.01% | 0.00% | 0.00% | 0.00% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.00% | 0.00% | 0.00% | 0.00% | 0.01% | 0.01% | 0.03% | 0.00% | 0.30% | 0.01% | 0.01% | 0.20% |
| SaKKH-Cas9 with | | | | | | | | | | | | | |
| saG4 gRNA | | | | | | | | | | | | | |
| G1333 DddAtox-C- | A | 0.19% | 0.04% | 0.00% | 0.00% | 0.00% | 0.00% | 0.00% | 99.93% | 99.63% | 0.00% | 0.00% | 0.00% |
| SaKKH-Cas9 with | | | | | | | | | | | | | |
| saG4 gRNA | | | | | | | | | | | | | |
| G1333 DddAtox-C- | T | 0.21% | 0.03% | 0.01% | 0.01% | 0.01% | 0.01% | 99.92% | 0.01% | 0.00% | 0.01% | 0.01% | 0.00% |
| SaKKH-Cas9 with | | | | | | | | | | | | | |
| saG4 gRNA | | | | | | | | | | | | | |
| G1333 DddAtox-C- | C | 0.13% | 0.02% | 99.27% | 99.98% | 99.97% | 99.97% | 0.03% | 0.04% | 0.01% | 99.99% | 99.98% | 99.73% |
| SaKKH-Cas9 with | | | | | | | | | | | | | |
| saG4 gRNA | | | | | | | | | | | | | |
| G1333 DddAtox-C- | G | 99.47% | 99.91% | 0.72% | 0.01% | 0.00% | 0.00% | 0.00% | 0.02% | 0.01% | 0.00% | 0.00% | 0.00% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.00% | 0.00% | 0.00% | 0.00% | 0.01% | 0.01% | 0.04% | 0.00% | 0.35% | 0.00% | 0.01% | 0.26% |
| SaKKH-Cas9 with | | | | | | | | | | | | | |
| saG4 gRNA | | | | | | | | | | | | | |
| G1397 DddAtox-N- | A | 0.21% | 0.04% | 0.01% | 0.01% | 0.00% | 0.01% | 0.00% | 99.93% | 99.62% | 0.01% | 0.00% | 0.01% |
| SaKKH-Cas9 with | | | | | | | | | | | | | |
| saG4 gRNA | | | | | | | | | | | | | |
| G1397 DddAtox-N- | T | 0.23% | 0.03% | 0.00% | 0.01% | 0.01% | 0.01% | 99.94% | 0.02% | 0.01% | 0.00% | 0.01% | 0.00% |
| SaKKH-Cas9 with | | | | | | | | | | | | | |
| saG4 gRNA | | | | | | | | | | | | | |
| G1397 DddAtox-N- | C | 0.13% | 0.03% | 99.26% | 99.97% | 99.98% | 99.98% | 0.02% | 0.03% | 0.00% | 99.98% | 99.99% | 99.75% |
| SaKKH-Cas9 with | | | | | | | | | | | | | |
| saG4 gRNA | | | | | | | | | | | | | |
| G1397 DddAtox-N- | G | 99.43% | 99.91% | 0.73% | 0.01% | 0.00% | 0.00% | 0.00% | 0.01% | 0.01% | 0.00% | 0.00% | 0.00% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 0.00% | 0.00% | 0.00% | 0.00% | 0.01% | 0.01% | 0.03% | 0.01% | 0.36% | 0.01% | 0.01% | 0.23% |
| SaKKH-Cas9 with | | | | | | | | | | | | | |
| saG4 gRNA | | | | | | | | | | | | | |
| G1397 DddAtox-C- | A | 0.19% | 0.03% | 0.01% | 0.01% | 0.00% | 0.01% | 0.00% | 99.91% | 99.63% | 0.00% | 0.01% | 0.01% |
| SaKKH-Cas9 with | | | | | | | | | | | | | |
| saG4 gRNA | | | | | | | | | | | | | |
| G1397 DddAtox-C- | T | 0.25% | 0.05% | 0.01% | 0.01% | 0.01% | 0.01% | 99.92% | 0.01% | 0.00% | 0.01% | 0.01% | 0.01% |
| SaKKH-Cas9 with | | | | | | | | | | | | | |
| saG4 gRNA | | | | | | | | | | | | | |
| G1397 DddAtox-C- | C | 0.16% | 0.01% | 99.19% | 99.97% | 99.98% | 99.97% | 0.03% | 0.04% | 0.01% | 99.98% | 99.98% | 99.73% |
| SaKKH-Cas9 with | | | | | | | | | | | | | |
| saG4 gRNA | | | | | | | | | | | | | |
| G1397 DddAtox-C- | G | 99.40% | 99.91% | 0.79% | 0.02% | 0.00% | 0.00% | 0.01% | 0.02% | 0.01% | 0.00% | 0.00% | 0.00% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.00% | 0.00% | 0.00% | 0.00% | 0.01% | 0.01% | 0.04% | 0.01% | 0.35% | 0.01% | 0.01% | 0.26% |
| SaKKH-Cas9 with | | | | | | | | | | | | | |
| saG4 gRNA | | | | | | | | | | | | | |
| Batch | Nucleotide | T | A | T | G | T | A | G | C | C | T | C | A |
| G1333 DddAtox-N- | A | 0.00% | 99.90% | 0.00% | 0.05% | 0.01% | 99.94% | 0.03% | 0.00% | 0.00% | 0.01% | 0.01% | 99.96% |
| SaKKH-Cas9 with | | | | | | | | | | | | | |
| saG4 gRNA | | | | | | | | | | | | | |
| G1333 DddAtox-N- | T | 99.97% | 0.01% | 99.98% | 0.03% | 99.97% | 0.01% | 0.04% | 0.01% | 0.01% | 99.98% | 0.01% | 0.00% |
| SaKKH-Cas9 with | | | | | | | | | | | | | |
| saG4 gRNA | | | | | | | | | | | | | |
| G1333 DddAtox-N- | C | 0.01% | 0.03% | 0.01% | 0.00% | 0.01% | 0.02% | 0.00% | 99.99% | 99.99% | 0.01% | 99.97% | 0.02% |
| SaKKH-Cas9 with | | | | | | | | | | | | | |
| saG4 gRNA | | | | | | | | | | | | | |
| G1333 DddAtox-N- | G | 0.00% | 0.02% | 0.00% | 99.92% | 0.00% | 0.03% | 99.93% | 0.00% | 0.00% | 0.00% | 0.00% | 0.01% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.02% | 0.04% | 0.01% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | A | 0.01% | 99.90% | 0.01% | 0.05% | 0.01% | 99.94% | 0.01% | 0.01% | 0.00% | 0.01% | 0.01% | 99.96% |
| SaKKH-Cas9 with | | | | | | | | | | | | | |
| saG4 gRNA | | | | | | | | | | | | | |
| G1333 DddAtox-C- | T | 99.97% | 0.02% | 99.97% | 0.03% | 99.98% | 0.00% | 0.05% | 0.01% | 0.01% | 99.99% | 0.02% | 0.01% |
| SaKKH-Cas9 with | | | | | | | | | | | | | |
| saG4 gRNA | | | | | | | | | | | | | |
| G1333 DddAtox-C- | C | 0.00% | 0.01% | 0.01% | 0.00% | 0.01% | 0.02% | 0.00% | 99.99% | 99.99% | 0.01% | 99.97% | 0.01% |
| SaKKH-Cas9 with | | | | | | | | | | | | | |
| saG4 gRNA | | | | | | | | | | | | | |
| G1333 DddAtox-C- | G | 0.00% | 0.03% | 0.00% | 99.92% | 0.00% | 0.04% | 99.94% | 0.00% | 0.00% | 0.00% | 0.00% | 0.02% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.02% | 0.04% | 0.01% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | A | 0.01% | 99.90% | 0.01% | 0.06% | 0.02% | 99.94% | 0.02% | 0.00% | 0.01% | 0.01% | 0.01% | 99.97% |
| SaKKH-Cas9 with | | | | | | | | | | | | | |
| saG4 gRNA | | | | | | | | | | | | | |
| G1397 DddAtox-N- | T | 99.95% | 0.01% | 99.97% | 0.04% | 99.97% | 0.01% | 0.03% | 0.00% | 0.01% | 99.97% | 0.01% | 0.01% |
| SaKKH-Cas9 with | | | | | | | | | | | | | |
| saG4 gRNA | | | | | | | | | | | | | |
| G1397 DddAtox-N- | C | 0.01% | 0.02% | 0.01% | 0.01% | 0.00% | 0.02% | 0.00% | 99.99% | 99.98% | 0.02% | 99.98% | 0.01% |
| SaKKH-Cas9 with | | | | | | | | | | | | | |
| saG4 gRNA | | | | | | | | | | | | | |
| G1397 DddAtox-N- | G | 0.00% | 0.01% | 0.00% | 99.90% | 0.00% | 0.03% | 99.95% | 0.00% | 0.00% | 0.01% | 0.00% | 0.02% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 0.03% | 0.06% | 0.01% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | A | 0.01% | 99.90% | 0.01% | 0.07% | 0.01% | 99.94% | 0.02% | 0.01% | 0.01% | 0.01% | 0.01% | 99.97% |
| SaKKH-Cas9 with | | | | | | | | | | | | | |
| saG4 gRNA | | | | | | | | | | | | | |
| G1397 DddAtox-C- | T | 99.95% | 0.01% | 99.96% | 0.04% | 99.97% | 0.01% | 0.05% | 0.01% | 0.01% | 99.97% | 0.02% | 0.00% |
| SaKKH-Cas9 with | | | | | | | | | | | | | |
| saG4 gRNA | | | | | | | | | | | | | |
| G1397 DddAtox-C- | C | 0.01% | 0.03% | 0.01% | 0.00% | 0.01% | 0.03% | 0.00% | 99.99% | 99.99% | 0.01% | 99.97% | 0.02% |
| SaKKH-Cas9 with | | | | | | | | | | | | | |
| saG4 gRNA | | | | | | | | | | | | | |
| G1397 DddAtox-C- | G | 0.00% | 0.03% | 0.00% | 99.89% | 0.00% | 0.03% | 99.93% | 0.00% | 0.00% | 0.01% | 0.00% | 0.01% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.03% | 0.03% | 0.02% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |

| | Nucle- | | | | | | | | | | | | | |
|---|---|---|---|---|---|---|---|---|---|---|---|---|---|---|
| Batch | otide | G | T | C | T | T | C | C | C | A | T | C | A | G |
| G1333 | A | 0.01% | 0.00% | 0.00% | 0.00% | 0.00% | 0.01% | 0.01% | 0.00% | 99.96% | 0.01% | 0.01% | 99.96% | 0.01% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1333 | T | 0.05% | 99.99% | 0.00% | 99.98% | 99.97% | 0.01% | 0.00% | 0.01% | 0.01% | 99.98% | 0.01% | 0.01% | 0.04% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1333 | C | 0.00% | 0.01% | 99.99% | 0.01% | 0.02% | 99.98% | 99.98% | 99.98% | 0.01% | 0.00% | 99.99% | 0.01% | 0.00% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1333 | G | 99.95% | 0.00% | 0.00% | 0.00% | 0.00% | 0.00% | 0.00% | 0.00% | 0.02% | 0.01% | 0.00% | 0.02% | 99.95% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1333 | A | 0.01% | 0.01% | 0.00% | 0.00% | 0.01% | 0.00% | 0.01% | 0.01% | 99.96% | 0.00% | 0.01% | 99.96% | 0.01% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1333 | T | 0.03% | 99.98% | 0.01% | 99.98% | 99.98% | 0.02% | 0.00% | 0.01% | 0.01% | 99.99% | 0.00% | 0.01% | 0.05% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1333 | C | 0.00% | 0.01% | 99.99% | 0.02% | 0.01% | 99.98% | 99.99% | 99.98% | 0.01% | 0.00% | 99.98% | 0.01% | 0.00% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1333 | G | 99.95% | 0.00% | 0.00% | 0.00% | 0.01% | 0.00% | 0.00% | 0.00% | 0.03% | 0.00% | 0.00% | 0.02% | 99.94% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1397 | A | 0.01% | 0.00% | 0.01% | 0.00% | 0.00% | 0.01% | 0.00% | 0.00% | 99.95% | 0.00% | 0.00% | 99.96% | 0.01% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1397 | T | 0.04% | 99.99% | 0.00% | 99.98% | 99.97% | 0.01% | 0.01% | 0.01% | 0.00% | 99.99% | 0.01% | 0.01% | 0.04% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1397 | C | 0.01% | 0.01% | 99.99% | 0.01% | 0.02% | 99.98% | 99.98% | 99.98% | 0.01% | 0.01% | 99.99% | 0.01% | 0.00% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1397 | G | 99.95% | 0.00% | 0.00% | 0.00% | 0.00% | 0.01% | 0.00% | 0.00% | 0.04% | 0.00% | 0.00% | 0.02% | 99.94% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | | |
| N-SaKKH- | | | | | | | | | | | | | | |
| Cas9 with | | | | | | | | | | | | | | |
| saG4 gRNA | | | | | | | | | | | | | | |
| G1397 | A | 0.00% | 0.01% | 0.00% | 0.01% | 0.01% | 0.00% | 0.01% | 0.01% | 99.96% | 0.01% | 0.00% | 99.97% | 0.01% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1397 | T | 0.04% | 99.98% | 0.00% | 99.98% | 99.97% | 0.01% | 0.01% | 0.01% | 0.01% | 99.98% | 0.01% | 0.00% | 0.03% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1397 | C | 0.00% | 0.01% | 99.99% | 0.02% | 0.02% | 99.98% | 99.98% | 99.98% | 0.01% | 0.01% | 99.98% | 0.01% | 0.00% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1397 | G | 99.95% | 0.00% | 0.00% | 0.00% | 0.01% | 0.00% | 0.00% | 0.00% | 0.02% | 0.01% | 0.00% | 0.02% | 99.96% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | | |
| C-SaKKH- | | | | | | | | | | | | | | |
| Cas9 with | | | | | | | | | | | | | | |
| saG4 gRNA | | | | | | | | | | | | | | |

| | Nucle- | | | | | | | | | | | |
|---|---|---|---|---|---|---|---|---|---|---|---|---|
| Batch | otide | G | C | T | C | T | C | A | G | C | T | C |
| G1333 | A | 0.01% | 0.00% | 0.00% | 0.01% | 0.00% | 0.00% | 99.97% | 0.01% | 0.00% | 0.01% | 0.01% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1333 | T | 0.03% | 0.00% | 99.99% | 0.01% | 99.99% | 0.01% | 0.00% | 0.01% | 0.01% | 99.98% | 0.01% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1333 | C | 0.00% | 100.00% | 0.01% | 99.98% | 0.01% | 99.98% | 0.01% | 0.00% | 99.99% | 0.01% | 99.98% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1333 | G | 99.96% | 0.00% | 0.00% | 0.00% | 0.00% | 0.00% | 0.02% | 99.97% | 0.00% | 0.00% | 0.00% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1333 | N | 0.00% | 0.00% | 0.00% | 0.00% | 0.00% | 0.00% | 0.00% | 0.00% | 0.00% | 0.00% | 0.00% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1333 | — | 0.00% | 0.00% | 0.00% | 0.00% | 0.00% | 0.00% | 0.00% | 0.00% | 0.00% | 0.00% | 0.00% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1333 | A | 0.01% | 0.00% | 0.00% | 0.01% | 0.00% | 0.01% | 99.97% | 0.00% | 0.00% | 0.01% | 0.00% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1333 | T | 0.02% | 0.01% | 99.99% | 0.01% | 99.98% | 0.02% | 0.00% | 0.01% | 0.01% | 99.97% | 0.01% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1333 | C | 0.00% | 99.98% | 0.00% | 99.99% | 0.01% | 99.98% | 0.01% | 0.00% | 99.99% | 0.02% | 99.98% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1333 | G | 99.97 | 0.00% | 0.00% | 0.00% | 0.00% | 0.00% | 0.022% | 99.98% | 0.00% | 0.00% | 0.00% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1333 | N | 0.00% | 0.00% | 0.00% | 0.00% | 0.00% | 0.00% | 0.00% | 0.00% | 0.00% | 0.00% | 0.00% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1333 | — | 0.00% | 0.00% | 0.00% | 0.00% | 0.00% | 0.00% | 0.00% | 0.00% | 0.00% | 0.00% | 0.00% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1397 | A | 0.01% | 0.00% | 0.00% | 0.00% | 0.00% | 0.00% | 99.98% | 0.01% | 0.00% | 0.00% | 0.00% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1397 | T | 0.01% | 0.00% | 99.99% | 0.01% | 99.99% | 0.01% | 0.00% | 0.01% | 0.01% | 99.98% | 0.01% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1397 | C | 0.01% | 99.99% | 0.01% | 99.99% | 0.01% | 99.98% | 0.01% | 0.00% | 99.98% | 0.01% | 99.98% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1397 | G | 99.97% | 0.00% | 0.00% | 0.00% | 0.00% | 0.00% | 0.01% | 99.98% | 0.00% | 0.01% | 0.00% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1397 | N | 0.00% | 0.00% | 0.00% | 0.00% | 0.00% | 0.00% | 0.00% | 0.00% | 0.00% | 0.00% | 0.00% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1397 | — | 0.00% | 0.00% | 0.00% | 0.00% | 0.00% | 0.00% | 0.00% | 0.00% | 0.00% | 0.00% | 0.00% |
| DddAtox- | | | | | | | | | | | | |
| N-SaKKH- | | | | | | | | | | | | |
| Cas9 with | | | | | | | | | | | | |
| saG4 gRNA | | | | | | | | | | | | |
| G1397 | A | 0.01% | 0.00% | 0.00% | 0.00% | 0.01% | 0.01% | 99.96% | 0.01% | 0.00% | 0.01% | 0.00% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1397 | T | 0.01% | 0.00% | 99.98% | 0.00% | 99.98% | 0.01% | 0.00% | 0.01% | 0.01% | 99.97% | 0.01% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1397 | C | 0.01% | 99.99% | 0.02% | 100.00% | 0.01% | 99.98% | 0.01% | 0.00% | 99.98% | 0.01% | 99.98% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1397 | G | 99.97% | 0.00% | 0.00% | 0.00% | 0.00% | 0.00% | 0.02% | 99.97% | 0.00% | 0.01% | 0.00% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1397 | N | 0.00% | 0.00% | 0.00% | 0.00% | 0.00% | 0.00% | 0.00% | 0.00% | 0.00% | 0.00% | 0.00% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |
| G1397 | — | 0.00% | 0.00% | 0.00% | 0.00% | 0.00% | 0.00% | 0.00% | 0.00% | 0.00% | 0.00% | 0.00% |
| DddAtox- | | | | | | | | | | | | |
| C-SaKKH- | | | | | | | | | | | | |
| Cas9 with | | | | | | | | | | | | |
| saG4 gRNA | | | | | | | | | | | | |

| | Nucle- | | | | | | | | | | | | |
|---|---|---|---|---|---|---|---|---|---|---|---|---|---|
| Batch | otide | A | G | C | C | T | G | A | G | T | G | T | T |
| G1333 | A | 99.97% | 0.02% | 0.00% | 0.00% | 0.00% | 0.01% | 99.96% | 0.00% | 0.01% | 0.01% | 0.01% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | T | 0.00% | 0.01% | 0.01% | 0.01% | 99.98% | 0.02% | 0.00% | 0.01% | 99.96% | 0.01% | 99.97% | 99.97% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | C | 0.01% | 0.00% | 99.99% | 99.99% | 0.01% | 0.00% | 0.01% | 0.00% | 0.01% | 0.00% | 0.01% | 0.02% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | G | 0.02% | 99.97% | 0.00% | 0.00% | 0.00% | 99.97% | 0.02% | 99.98% | 0.02% | 99.97% | 0.01% | 0.01% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | A | 99.96% | 0.01% | 0.00% | 0.00% | 0.00% | 0.01% | 99.98% | 0.00% | 0.01% | 0.01% | 0.00% | 0.01% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | T | 0.00% | 0.01% | 0.01% | 0.00% | 99.98% | 0.01% | 0.00% | 0.01% | 99.96% | 0.01% | 99.96% | 99.97% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | C | 0.01% | 0.00% | 99.99% | 100.00% | 0.02% | 0.00% | 0.00% | 0.00% | 0.01% | 0.00% | 0.02% | 0.02% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | G | 0.03% | 99.98% | 0.00% | 0.00% | 0.00% | 99.99% | 0.02% | 99.99% | 0.02% | 99.98% | 0.01% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | A | 99.97% | 0.01% | 0.00% | 0.01% | 0.00% | 0.01% | 99.97% | 0.01% | 0.01% | 0.01% | 0.01% | 0.01% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | T | 0.01% | 0.01% | 0.00% | 0.01% | 99.98% | 0.02% | 0.00% | 0.01% | 99.97% | 0.01% | 99.96% | 99.97% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | C | 0.01% | 0.00% | 100.00% | 99.99% | 0.01% | 0.00% | 0.01% | 0.00% | 0.01% | 0.00% | 0.02% | 0.01% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | G | 0.01% | 99.98% | 0.00% | 0.00% | 0.00% | 99.97% | 0.01% | 99.99% | 0.01% | 99.97% | 0.00% | 0.01% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | A | 99.97% | 0.01% | 0.00% | 0.00% | 0.01% | 0.01% | 99.97% | 0.01% | 0.01% | 0.00% | 0.01% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | T | 0.01% | 0.01% | 0.01% | 0.01% | 99.97% | 0.01% | 0.01% | 0.00% | 99.94% | 0.01% | 99.97% | 99.97% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | C | 0.01% | 0.00% | 99.99% | 99.99% | 0.02% | 0.01% | 0.01% | 0.00% | 0.02% | 0.01% | 0.02% | 0.02% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | G | 0.01% | 99.98% | 0.01% | 0.00% | 0.00% | 99.97% | 0.01% | 99.99% | 0.03% | 99.98% | 0.01% | 0.01% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |

| | Nucle- | | | | | | | | | | | | |
|---|---|---|---|---|---|---|---|---|---|---|---|---|---|
| Batch | otide | G | A | G | G | C | C | C | C | A | G | T | G |
| G1333 | A | 0.02% | 99.95% | 0.01% | 0.01% | 0.00% | 0.01% | 0.01% | 0.01% | 99.94% | 0.00% | 0.01% | 0.01% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | T | 0.01% | 0.01% | 0.01% | 0.01% | 0.01% | 0.00% | 0.01% | 0.01% | 0.01% | 0.01% | 99.96% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | C | 0.00% | 0.03% | 0.00% | 0.00% | 99.97% | 99.99% | 99.97% | 99.96% | 0.02% | 0.00% | 0.02% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | G | 99.95% | 0.01% | 99.96% | 99.97% | 0.00% | 0.00% | 0.00% | 0.00% | 0.03% | 99.98% | 0.01% | 99.98% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | — | 0.01% | 0.01% | 0.01% | 0.00% | 0.00% | 0.00% | 0.00% | 0.02% | 0.00% | 0.00% | 0.00% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1333 | A | 0.02% | 99.96% | 0.01% | 0.01% | 0.00% | 0.01% | 0.01% | 0.01% | 99.96% | 0.00% | 0.02% | 0.02% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | T | 0.01% | 0.01% | 0.02% | 0.01% | 0.00% | 0.01% | 0.01% | 0.01% | 0.01% | 0.01% | 99.94% | 0.02% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | C | 0.00% | 0.02% | 0.00% | 0.00% | 99.99% | 99.98% | 99.98% | 99.97% | 0.02% | 0.00% | 0.02% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | G | 99.96% | 0.01% | 99.97% | 99.97% | 0.00% | 0.00% | 0.00% | 0.00% | 0.02% | 99.99% | 0.02% | 99.96% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1333 | — | 0.00% | 0.00% | 0.00% | 0.00% | 0.00% | 0.00% | 0.00% | 0.01% | 0.00% | 0.00% | 0.00% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | A | 0.02% | 99.95% | 0.01% | 0.02% | 0.00% | 0.01% | 0.01% | 0.01% | 99.98% | 0.01% | 0.02% | 0.01% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | T | 0.01% | 0.00% | 0.02% | 0.00% | 0.01% | 0.01% | 0.01% | 0.02% | 0.00% | 0.01% | 99.93% | 0.01% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | C | 0.00% | 0.02% | 0.00% | 0.00% | 99.97% | 99.98% | 99.98% | 99.95% | 0.00% | 0.00% | 0.02% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | G | 99.97% | 0.02% | 99.97% | 99.97% | 0.01% | 0.00% | 0.01% | 0.01% | 0.01% | 99.98% | 0.03% | 99.98% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | — | 0.00% | 0.00% | 0.00% | 0.00% | 0.00% | 0.00% | 0.00% | 0.02% | 0.00% | 0.00% | 0.00% | 0.00% |
| DddAtox- | | | | | | | | | | | | | |
| N-SaKKH- | | | | | | | | | | | | | |
| Cas9 with | | | | | | | | | | | | | |
| saG4 gRNA | | | | | | | | | | | | | |
| G1397 | A | 0.01% | 99.95% | 0.01% | 0.01% | 0.01% | 0.01% | 0.01% | 0.02% | 99.96% | 0.00% | 0.01% | 0.01% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | T | 0.00% | 0.01% | 0.01% | 0.01% | 0.01% | 0.01% | 0.00% | 0.00% | 0.00% | 0.01% | 99.95% | 0.01% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | C | 0.00% | 0.03% | 0.00% | 0.00% | 99.98% | 99.98% | 99.98% | 99.96% | 0.01% | 0.00% | 0.02% | 0.01% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | G | 99.98% | 0.01% | 99.97% | 99.99% | 0.00% | 0.01% | 0.00% | 0.00% | 0.02% | 99.98% | 0.02% | 99.97% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox- | | | | | | | | | | | | | |
| C-SaKKH- | | | | | | | | | | | | | |
| Cas9 with | | | | | | | | | | | | | |
| saG4 gRNA | | | | | | | | | | | | | |

| | Nucle- | | | | | | | | | | | | |
|---|---|---|---|---|---|---|---|---|---|---|---|---|---|
| Batch | otide | G | C | T | G | C | T | C | T | G | G | G | G |
| G1333 DddAtox-N- | A | 0.00% | 0.01% | 0.01% | 0.02% | 0.01% | 0.00% | 0.01% | 0.01% | 0.01% | 0.01% | 0.01% | 0.01% |
| SaKKH-Cas9 with | | | | | | | | | | | | | |
| saG4 gRNA | | | | | | | | | | | | | |
| G1333 DddAtox-N- | T | 0.01% | 0.01% | 99.97% | 0.02% | 0.00% | 99.97% | 0.00% | 99.97% | 0.02% | 0.02% | 0.02% | 0.01% |
| SaKKH-Cas9 with | | | | | | | | | | | | | |
| saG4 gRNA | | | | | | | | | | | | | |
| G1333 DddAtox-N- | C | 0.00% | 99.98% | 0.02% | 0.00% | 99.99% | 0.01% | 99.99% | 0.02% | 0.01% | 0.00% | 0.01% | 0.01% |
| SaKKH-Cas9 with | | | | | | | | | | | | | |
| saG4 gRNA | | | | | | | | | | | | | |
| G1333 DddAtox-N- | G | 99.98% | 0.00% | 0.00% | 99.96% | 0.00% | 0.01% | 0.00% | 0.00% | 99.96% | 99.97% | 99.95% | 99.97% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.00% | 0.00% | 0.00% | 0.00% | 0.00% | 0.00% | 0.00% | 0.00% | 0.00% | 0.00% | 0.00% | 0.01% |
| SaKKH-Cas9 with | | | | | | | | | | | | | |
| saG4 gRNA | | | | | | | | | | | | | |
| G1333 DddAtox-C- | A | 0.01% | 0.01% | 0.01% | 0.01% | 0.01% | 0.00% | 0.01% | 0.01% | 0.01% | 0.02% | 0.01% | 0.01% |
| SaKKH-Cas9 with | | | | | | | | | | | | | |
| saG4 gRNA | | | | | | | | | | | | | |
| G1333 DddAtox-C- | T | 0.02% | 0.01% | 99.98% | 0.02% | 0.01% | 99.98% | 0.00% | 99.97% | 0.02% | 0.03% | 0.02% | 0.01% |
| SaKKH-Cas9 with | | | | | | | | | | | | | |
| saG4 gRNA | | | | | | | | | | | | | |
| G1333 DddAtox-C- | C | 0.00% | 99.98% | 0.01% | 0.00% | 99.98% | 0.02% | 99.98% | 0.02% | 0.00% | 0.00% | 0.01% | 0.00% |
| SaKKH-Cas9 with | | | | | | | | | | | | | |
| saG4 gRNA | | | | | | | | | | | | | |
| G1333 DddAtox-C- | G | 99.96% | 0.00% | 0.00% | 99.97% | 0.00% | 0.00% | 0.00% | 0.00% | 99.97% | 99.95% | 99.77% | 99.77% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.00% | 0.00% | 0.00% | 0.00% | 0.00% | 0.00% | 0.00% | 0.00% | 0.00% | 0.00% | 0.20% | 0.20% |
| SaKKH-Cas9 with | | | | | | | | | | | | | |
| saG4 gRNA | | | | | | | | | | | | | |
| G1397 DddAtox-N- | A | 0.00% | 0.01% | 0.03% | 0.02% | 0.03% | 0.02% | 0.01% | 0.00% | 0.01% | 0.03% | 0.03% | 0.02% |
| SaKKH-Cas9 with | | | | | | | | | | | | | |
| saG4 gRNA | | | | | | | | | | | | | |
| G1397 DddAtox-N- | T | 0.01% | 0.00% | 99.94% | 0.01% | 0.01% | 99.95% | 0.01% | 99.98% | 0.02% | 0.02% | 0.01% | 0.01% |
| SaKKH-Cas9 with | | | | | | | | | | | | | |
| saG4 gRNA | | | | | | | | | | | | | |
| G1397 DddAtox-N- | C | 0.01% | 99.98% | 0.01% | 0.00% | 99.96% | 0.01% | 99.98% | 0.01% | 0.01% | 0.01% | 0.01% | 0.00% |
| SaKKH-Cas9 with | | | | | | | | | | | | | |
| saG4 gRNA | | | | | | | | | | | | | |
| G1397 DddAtox-N- | G | 99.98% | 0.01% | 0.01% | 99.96% | 0.00% | 0.01% | 0.00% | 0.01% | 99.96% | 99.95% | 99.34% | 97.81% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 0.00% | 0.00% | 0.00% | 0.00% | 0.00% | 0.00% | 0.00% | 0.00% | 0.00% | 0.00% | 0.60% | 2.15% |
| SaKKH-Cas9 with | | | | | | | | | | | | | |
| saG4 gRNA | | | | | | | | | | | | | |
| G1397 DddAtox-C- | A | 0.01% | 0.00% | 0.01% | 0.02% | 0.01% | 0.00% | 0.01% | 0.01% | 0.01% | 0.01% | 0.01% | 0.01% |
| SaKKH-Cas9 with | | | | | | | | | | | | | |
| saG4 gRNA | | | | | | | | | | | | | |
| G1397 DddAtox-C- | T | 0.02% | 0.01% | 99.97% | 0.02% | 0.01% | 99.99% | 0.01% | 99.96% | 0.02% | 0.02% | 0.02% | 0.01% |
| SaKKH-Cas9 with | | | | | | | | | | | | | |
| saG4 gRNA | | | | | | | | | | | | | |
| G1397 DddAtox-C- | C | 0.01% | 99.98% | 0.02% | 0.00% | 99.98% | 0.01% | 99.99% | 0.02% | 0.00% | 0.00% | 0.01% | 0.00% |
| SaKKH-Cas9 with | | | | | | | | | | | | | |
| saG4 gRNA | | | | | | | | | | | | | |
| G1397 DddAtox-C- | G | 99.97% | 0.00% | 0.00% | 99.96% | 0.00% | 0.01% | 0.00% | 0.00% | 99.97% | 99.97% | 99.96% | 99.97% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.00% | 0.00% | 0.00% | 0.00% | 0.00% | 0.00% | 0.00% | 0.00% | 0.00% | 0.00% | 0.00% | 0.01% |
| SaKKH-Cas9 with | | | | | | | | | | | | | |
| saG4 gRNA | | | | | | | | | | | | | |
| | Nucle- | | | | | | | | | | | | |
| Batch | otide | G | C | C | T | C | C | T | G | A | G | T | T |
| G1333 DddAtox-N- | A | 0.01% | 0.00% | 0.02% | 0.01% | 0.00% | 0.01% | 0.01% | 0.02% | 99.93% | 0.00% | 0.00% | 0.00% |
| SaKKH-Cas9 with | | | | | | | | | | | | | |
| saG4 gRNA | | | | | | | | | | | | | |
| G1333 DddAtox-N- | T | 0.02% | 0.00% | 0.00% | 99.97% | 0.00% | 0.01% | 99.96% | 0.02% | 0.00% | 0.00% | 99.97% | 99.97% |
| SaKKH-Cas9 with | | | | | | | | | | | | | |
| saG4 gRNA | | | | | | | | | | | | | |
| G1333 DddAtox-N- | C | 0.00% | 99.99% | 99.97% | 0.01% | 99.99% | 99.98% | 0.02% | 0.00% | 0.04% | 0.00% | 0.01% | 0.02% |
| SaKKH-Cas9 with | | | | | | | | | | | | | |
| saG4 gRNA | | | | | | | | | | | | | |
| G1333 DddAtox-N- | G | 99.95% | 0.00% | 0.00% | 0.00% | 0.00% | 0.00% | 0.00% | 99.95% | 0.02% | 99.98% | 0.01% | 0.00% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.02% | 0.00% | 0.01% | 0.01% | 0.01% | 0.01% | 0.01% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | A | 0.01% | 0.00% | 0.02% | 0.00% | 0.01% | 0.00% | 0.01% | 0.01% | 99.75% | 0.00% | 0.01% | 0.00% |
| SaKKH-Cas9 with | | | | | | | | | | | | | |
| saG4 gRNA | | | | | | | | | | | | | |
| G1333 DddAtox-C- | T | 0.01% | 0.01% | 0.01% | 99.77% | 0.01% | 0.00% | 99.77% | 0.01% | 0.01% | 0.01% | 99.77% | 99.78% |
| SaKKH-Cas9 with | | | | | | | | | | | | | |
| saG4 gRNA | | | | | | | | | | | | | |
| G1333 DddAtox-C- | C | 0.00% | 99.78% | 99.77% | 0.01% | 99.78% | 99.79% | 0.02% | 0.00% | 0.03% | 0.00% | 0.01% | 0.01% |
| SaKKH-Cas9 with | | | | | | | | | | | | | |
| saG4 gRNA | | | | | | | | | | | | | |
| G1333 DddAtox-C- | G | 99.74% | 0.00% | 0.00% | 0.00% | 0.00% | 0.00% | 0.00% | 99.77% | 0.01% | 99.79% | 0.01% | 0.00% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.24% | 0.20% | 0.20% | 0.20% | 0.20% | 0.20% | 0.21% | 0.20% | 0.20% | 0.20% | 0.20% | 0.20% |
| SaKKH-Cas9 with | | | | | | | | | | | | | |
| saG4 gRNA | | | | | | | | | | | | | |
| G1397 DddAtox-N- | A | 0.00% | 0.03% | 0.01% | 0.01% | 0.00% | 0.01% | 0.00% | 0.01% | 97.73% | 0.01% | 0.00% | 0.00% |
| SaKKH-Cas9 with | | | | | | | | | | | | | |
| saG4 gRNA | | | | | | | | | | | | | |
| G1397 DddAtox-N- | T | 0.01% | 0.01% | 0.01% | 97.80% | 0.01% | 0.01% | 97.82% | 0.01% | 0.01% | 0.01% | 97.75% | 97.76% |
| SaKKH-Cas9 with | | | | | | | | | | | | | |
| saG4 gRNA | | | | | | | | | | | | | |
| G1397 DddAtox-N- | C | 0.01% | 97.81% | 97.80% | 0.01% | 97.84% | 97.83% | 0.02% | 0.00% | 0.03% | 0.00% | 0.03% | 0.01% |
| SaKKH-Cas9 with | | | | | | | | | | | | | |
| saG4 gRNA | | | | | | | | | | | | | |
| G1397 DddAtox-N- | G | 97.80% | 0.01% | 0.02% | 0.03% | 0.00% | 0.00% | 0.00% | 97.83% | 0.03% | 97.78% | 0.01% | 0.00% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 2.18% | 2.15% | 2.15% | 2.15% | 2.15% | 2.15% | 2.15% | 2.15% | 2.20% | 2.20% | 2.20% | 2.22% |
| SaKKH-Cas9 with | | | | | | | | | | | | | |
| saG4 gRNA | | | | | | | | | | | | | |
| G1397 DddAtox-C- | A | 0.01% | 0.00% | 0.01% | 0.01% | 0.00% | 0.00% | 0.01% | 0.01% | 99.93% | 0.01% | 0.01% | 0.00% |
| SaKKH-Cas9 with | | | | | | | | | | | | | |
| saG4 gRNA | | | | | | | | | | | | | |
| G1397 DddAtox-C- | T | 0.01% | 0.01% | 0.01% | 99.97% | 0.01% | 0.01% | 99.97% | 0.01% | 0.01% | 0.01% | 99.96% | 99.99% |
| SaKKH-Cas9 with | | | | | | | | | | | | | |
| saG4 gRNA | | | | | | | | | | | | | |
| G1397 DddAtox-C- | C | 0.01% | 99.98% | 99.98% | 0.01% | 99.98% | 99.99% | 0.01% | 0.00% | 0.03% | 0.00% | 0.01% | 0.01% |
| SaKKH-Cas9 with | | | | | | | | | | | | | |
| saG4 gRNA | | | | | | | | | | | | | |
| G1397 DddAtox-C- | G | 99.93% | 0.00% | 0.00% | 0.01% | 0.00% | 0.00% | 0.00% | 99.98% | 0.03% | 99.98% | 0.01% | 0.00% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.04% | 0.01% | 0.01% | 0.01% | 0.01% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |

| Batch | Nucleotide | T | C | T | C | A | T | C | T | G | T | G | C |
|---|---|---|---|---|---|---|---|---|---|---|---|---|---|
| G1333 DddAtox-N- | A | 0.00% | 0.01% | 0.02% | 0.01% | 99.97% | 0.00% | 0.01% | 0.01% | 0.01% | 0.02% | 0.03% | 0.01% |
| SaKKH-Cas9 with | | | | | | | | | | | | | |
| saG4 gRNA | | | | | | | | | | | | | |
| G1333 DddAtox-N- | T | 99.98% | 0.01% | 99.96% | 0.01% | 0.00% | 99.99% | 0.00% | 99.97% | 0.02% | 99.93% | 0.01% | 0.00% |
| SaKKH-Cas9 with | | | | | | | | | | | | | |
| saG4 gRNA | | | | | | | | | | | | | |
| G1333 DddAtox-N- | C | 0.01% | 99.98% | 0.02% | 99.97% | 0.01% | 0.01% | 99.98% | 0.01% | 0.00% | 0.02% | 0.00% | 99.97% |
| SaKKH-Cas9 with | | | | | | | | | | | | | |
| saG4 gRNA | | | | | | | | | | | | | |
| G1333 DddAtox-N- | G | 0.00% | 0.00% | 0.00% | 0.00% | 0.01% | 0.00% | 0.00% | 0.00% | 99.96% | 0.01% | 99.95% | 0.00% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.00% | 0.00% | 0.00% | 0.00% | 0.00% | 0.00% | 0.00% | 0.01% | 0.01% | 0.01% | 0.01% | 0.01% |
| SaKKH-Cas9 with | | | | | | | | | | | | | |
| saG4 gRNA | | | | | | | | | | | | | |
| G1333 DddAtox-C- | A | 0.00% | 0.00% | 0.01% | 0.01% | 99.77% | 0.00% | 0.01% | 0.01% | 0.01% | 0.02% | 0.03% | 0.01% |
| SaKKH-Cas9 with | | | | | | | | | | | | | |
| saG4 gRNA | | | | | | | | | | | | | |
| G1333 DddAtox-C- | T | 99.78% | 0.00% | 99.77% | 0.01% | 0.00% | 99.79% | 0.01% | 99.78% | 0.02% | 99.73% | 0.02% | 0.01% |
| SaKKH-Cas9 with | | | | | | | | | | | | | |
| saG4 gRNA | | | | | | | | | | | | | |
| G1333 DddAtox-C- | C | 0.01% | 99.79% | 0.02% | 99.78% | 0.01% | 0.01% | 99.78% | 0.01% | 0.00% | 0.02% | 0.00% | 99.78% |
| SaKKH-Cas9 with | | | | | | | | | | | | | |
| saG4 gRNA | | | | | | | | | | | | | |
| G1333 DddAtox-C- | G | 0.00% | 0.00% | 0.00% | 0.00% | 0.02% | 0.00% | 0.00% | 0.00% | 99.77% | 0.02% | 99.74% | 0.00% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.20% | 0.20% | 0.20% | 0.20% | 0.20% | 0.20% | 0.20% | 0.20% | 0.20% | 0.20% | 0.21% | 0.21% |
| SaKKH-Cas9 with | | | | | | | | | | | | | |
| saG4 gRNA | | | | | | | | | | | | | |
| G1397 DddAtox-N- | A | 0.00% | 0.01% | 0.01% | 0.01% | 97.75% | 0.00% | 0.01% | 0.03% | 0.02% | 0.03% | 0.04% | 0.01% |
| SaKKH-Cas9 with | | | | | | | | | | | | | |
| saG4 gRNA | | | | | | | | | | | | | |
| G1397 DddAtox-N- | T | 97.76% | 0.01% | 97.75% | 0.01% | 0.01% | 97.42% | 0.01% | 96.67% | 0.01% | 96.65% | 0.01% | 0.02% |
| SaKKH-Cas9 with | | | | | | | | | | | | | |
| saG4 gRNA | | | | | | | | | | | | | |
| G1397 DddAtox-N- | C | 0.00% | 97.78% | 0.03% | 97.75% | 0.01% | 0.01% | 97.43% | 0.01% | 0.00% | 0.02% | 0.00% | 96.67% |
| SaKKH-Cas9 with | | | | | | | | | | | | | |
| saG4 gRNA | | | | | | | | | | | | | |
| G1397 DddAtox-N- | G | 0.01% | 0.01% | 0.01% | 0.02% | 0.02% | 0.03% | 0.01% | 0.01% | 96.68% | 0.01% | 96.67% | 0.02% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 2.22% | 2.21% | 2.21% | 2.21% | 2.21% | 2.54% | 2.54% | 3.28% | 3.28% | 3.28% | 3.28% | 3.28% |
| SaKKH-Cas9 with | | | | | | | | | | | | | |
| saG4 gRNA | | | | | | | | | | | | | |
| G1397 DddAtox-C- | A | 0.00% | 0.01% | 0.01% | 0.00% | 99.97% | 0.00% | 0.01% | 0.01% | 0.01% | 0.03% | 0.02% | 0.00% |
| SaKKH-Cas9 with | | | | | | | | | | | | | |
| saG4 gRNA | | | | | | | | | | | | | |
| G1397 DddAtox-C- | T | 99.98% | 0.01% | 99.97% | 0.01% | 0.00% | 99.99% | 0.01% | 99.97% | 0.01% | 99.94% | 0.01% | 0.01% |
| SaKKH-Cas9 with | | | | | | | | | | | | | |
| saG4 gRNA | | | | | | | | | | | | | |
| G1397 DddAtox-C- | C | 0.01% | 99.98% | 0.01% | 99.98% | 0.01% | 0.00% | 99.98% | 0.01% | 0.00% | 0.02% | 0.00% | 99.98% |
| SaKKH-Cas9 with | | | | | | | | | | | | | |
| saG4 gRNA | | | | | | | | | | | | | |
| G1397 DddAtox-C- | G | 0.00% | 0.00% | 0.00% | 0.00% | 0.02% | 0.00% | 0.00% | 0.01% | 99.97% | 0.01% | 99.96% | 0.00% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| Batch | Nucleotide | C | C | C | T | C | C | C | T | C | C | C | T |
| G1333 DddAtox-N- | A | 0.00% | 0.02% | 0.02% | 0.01% | 0.01% | 0.01% | 0.02% | 0.01% | 0.00% | 0.02% | 0.02% | 0.01% |
| SaKKH-Cas9 with | | | | | | | | | | | | | |
| saG4 gRNA | | | | | | | | | | | | | |
| G1333 DddAtox-N- | T | 0.01% | 0.01% | 0.01% | 99.96% | 0.02% | 0.02% | 0.01% | 99.97% | 0.01% | 0.01% | 0.00% | 99.68% |
| SaKKH-Cas9 with | | | | | | | | | | | | | |
| saG4 gRNA | | | | | | | | | | | | | |
| G1333 DddAtox-N- | C | 99.98% | 99.96% | 99.95% | 0.02% | 99.96% | 99.96% | 99.95% | 0.01% | 99.96% | 99.94% | 99.95% | 0.02% |
| SaKKH-Cas9 with | | | | | | | | | | | | | |
| saG4 gRNA | | | | | | | | | | | | | |
| G1333 DddAtox-N- | G | 0.00% | 0.00% | 0.00% | 0.00% | 0.00% | 0.00% | 0.00% | 0.00% | 0.00% | 0.00% | 0.00% | 0.01% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.01% | 0.01% | 0.01% | 0.01% | 0.01% | 0.01% | 0.02% | 0.01% | 0.02% | 0.02% | 0.03% | 0.2% |
| SaKKH-Cas9 with | | | | | | | | | | | | | |
| saG4 gRNA | | | | | | | | | | | | | |
| G1333 DddAtox-C- | A | 0.00% | 0.01% | 0.01% | 0.01% | 0.01% | 0.01% | 0.02% | 0.01% | 0.01% | 0.01% | 0.01% | 0.01% |
| SaKKH-Cas9 with | | | | | | | | | | | | | |
| saG4 gRNA | | | | | | | | | | | | | |
| G1333 DddAtox-C- | T | 0.01% | 0.01% | 0.01% | 99.75% | 0.02% | 0.03% | 0.01% | 99.77% | 0.02% | 0.01% | 0.01% | 99.72% |
| SaKKH-Cas9 with | | | | | | | | | | | | | |
| saG4 gRNA | | | | | | | | | | | | | |
| G1333 DddAtox-C- | C | 99.78% | 99.78% | 99.77% | 0.02% | 99.76% | 99.76% | 99.76% | 0.01% | 99.74% | 99.75% | 99.76% | 0.03% |
| SaKKH-Cas9 with | | | | | | | | | | | | | |
| saG4 gRNA | | | | | | | | | | | | | |
| G1333 DddAtox-C- | G | 0.00% | 0.00% | 0.00% | 0.01% | 0.00% | 0.00% | 0.00% | 0.01% | 0.01% | 0.00% | 0.00% | 0.02% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.20% | 0.20% | 0.21% | 0.21% | 0.20% | 0.20% | 0.21% | 0.20% | 0.22% | 0.22% | 0.23% | 0.23% |
| SaKKH-Cas9 with | | | | | | | | | | | | | |
| saG4 gRNA | | | | | | | | | | | | | |
| G1397 DddAtox-N- | A | 0.03% | 0.02% | 0.02% | 0.01% | 0.01% | 0.01% | 0.01% | 0.01% | 0.01% | 0.01% | 0.02% | 0.01% |
| SaKKH-Cas9 with | | | | | | | | | | | | | |
| saG4 gRNA | | | | | | | | | | | | | |
| G1397 DddAtox-N- | T | 0.01% | 0.02% | 0.01% | 96.67% | 0.03% | 0.02% | 0.01% | 96.6% | 0.02% | 0.01% | 0.01% | 97.37% |
| SaKKH-Cas9 with | | | | | | | | | | | | | |
| saG4 gRNA | | | | | | | | | | | | | |
| G1397 DddAtox-N- | C | 96.68% | 96.65% | 96.67% | 0.02% | 96.68% | 96.67% | 96.68% | 0.01% | 96.65% | 97.40% | 97.40% | 0.03% |
| SaKKH-Cas9 with | | | | | | | | | | | | | |
| saG4 gRNA | | | | | | | | | | | | | |
| G1397 DddAtox-N- | G | 0.00% | 0.03% | 0.00% | 0.01% | 0.00% | 0.01% | 0.00% | 0.01% | 0.00% | 0.01% | 0.00% | 0.02% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 3.28% | 3.28% | 3.29% | 3.28% | 3.28% | 3.30% | 3.30% | 3.30% | 3.31% | 2.57% | 2.58% | 2.57% |
| SaKKH-Cas9 with | | | | | | | | | | | | | |
| saG4 gRNA | | | | | | | | | | | | | |
| G1397 DddAtox-C- | A | 0.01% | 0.01% | 0.01% | 0.01% | 0.01% | 0.03% | 0.01% | 0.00% | 0.01% | 0.03% | 0.02% | 0.01% |
| SaKKH-Cas9 with | | | | | | | | | | | | | |
| saG4 gRNA | | | | | | | | | | | | | |
| G1397 DddAtox-C- | T | 0.01% | 0.01% | 0.01% | 99.93% | 0.06% | 0.02% | 0.00% | 99.86% | 0.03% | 0.01% | 0.00% | 99.81% |
| SaKKH-Cas9 with | | | | | | | | | | | | | |
| saG4 gRNA | | | | | | | | | | | | | |
| G1397 DddAtox-C- | C | 99.98% | 99.98% | 99.97% | 0.05% | 99.93% | 99.94% | 99.98% | 0.12% | 99.94% | 99.83% | 99.84% | 0.03% |
| SaKKH-Cas9 with | | | | | | | | | | | | | |
| saG4 gRNA | | | | | | | | | | | | | |
| G1397 DddAtox-C- | G | 0.00% | 0.00% | 0.00% | 0.00% | 0.00% | 0.00% | 0.00% | 0.00% | 0.00% | 0.00% | 0.00% | 0.01% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.00% | 0.00% | 0.00% | 0.00% | 0.00% | 0.00% | 0.01% | 0.01% | 0.02% | 0.13% | 0.14% | 0.13% |
| SaKKH-Cas9 with | | | | | | | | | | | | | |
| saG4 gRNA | | | | | | | | | | | | | |
| Batch | Nucleotide | G | G | C | C | C | A | G | G | T | G | A | A |
| G1333 DddAtox-N- | A | 0.02% | 0.02% | 0.01% | 0.01% | 0.03% | 99.18% | 0.01% | 0.01% | 0.03% | 0.01% | 99.16% | 99.12% |
| SaKKH-Cas9 with | | | | | | | | | | | | | |
| saG4 gRNA | | | | | | | | | | | | | |
| G1333 DddAtox-N- | T | 0.01% | 0.01% | 0.04% | 0.01% | 0.01% | 0.01% | 0.01% | 0.01% | 98.88% | 0.01% | 0.01% | 0.01% |
| SaKKH-Cas9 with | | | | | | | | | | | | | |
| saG4 gRNA | | | | | | | | | | | | | |
| G1333 DddAtox-N- | C | 0.00% | 0.01% | 99.32% | 99.26% | 99.24% | 0.04% | 0.00% | 0.01% | 0.06% | 0.00% | 0.04% | 0.03% |
| SaKKH-Cas9 with | | | | | | | | | | | | | |
| saG4 gRNA | | | | | | | | | | | | | |
| G1333 DddAtox-N- | G | 99.53% | 99.52% | 0.01% | 0.00% | 0.01% | 0.06% | 99.26% | 99.26% | 0.28% | 99.22% | 0.04% | 0.08% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.44% | 0.44% | 0.62% | 0.71% | 0.72% | 0.72% | 0.72% | 0.72% | 0.75% | 0.76% | 0.76% | 0.76% |
| SaKKH-Cas9 with | | | | | | | | | | | | | |
| saG4 gRNA | | | | | | | | | | | | | |
| G1333 DddAtox-C- | A | 0.01% | 0.01% | 0.01% | 0.01% | 0.03% | 99.65% | 0.01% | 0.01% | 0.03% | 0.01% | 99.58% | 99.55% |
| SaKKH-Cas9 with | | | | | | | | | | | | | |
| saG4 gRNA | | | | | | | | | | | | | |
| G1333 DddAtox-C- | T | 0.01% | 0.02% | 0.01% | 0.01% | 0.02% | 0.01% | 0.01% | 0.01% | 99.30% | 0.01% | 0.01% | 0.01% |
| SaKKH-Cas9 with | | | | | | | | | | | | | |
| saG4 gRNA | | | | | | | | | | | | | |
| G1333 DddAtox-C- | C | 0.00% | 0.00% | 99.79% | 99.74% | 99.70% | 0.03% | 0.00% | 0.00% | 0.10% | 0.00% | 0.04% | 0.03% |
| SaKKH-Cas9 with | | | | | | | | | | | | | |
| saG4 gRNA | | | | | | | | | | | | | |
| G1333 DddAtox-C- | G | 99.78% | 99.77% | 0.01% | 0.00% | 0.00% | 0.06% | 99.73% | 99.70% | 0.29% | 99.69% | 0.05% | 0.08% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.20% | 0.20% | 0.19% | 0.24% | 0.24% | 0.24% | 0.25% | 0.27% | 0.29% | 0.29% | 0.32% | 0.32% |
| SaKKH-Cas9 with | | | | | | | | | | | | | |
| saG4 gRNA | | | | | | | | | | | | | |
| G1397 DddAtox-N- | A | 0.02% | 0.01% | 0.03% | 0.03% | 0.02% | 97.25% | 0.02% | 0.01% | 0.05% | 0.01% | 97.22% | 97.19% |
| SaKKH-Cas9 with | | | | | | | | | | | | | |
| saG4 gRNA | | | | | | | | | | | | | |
| G1397 DddAtox-N- | T | 0.01% | 0.01% | 0.05% | 0.01% | 0.01% | 0.02% | 0.02% | 0.01% | 96.78% | 0.01% | 0.02% | 0.02% |
| SaKKH-Cas9 with | | | | | | | | | | | | | |
| saG4 gRNA | | | | | | | | | | | | | |
| G1397 DddAtox-N- | C | 0.01% | 0.01% | 97.39% | 97.44% | 97.31% | 0.04% | 0.00% | 0.02% | 0.25% | 0.02% | 0.06% | 0.05% |
| SaKKH-Cas9 with | | | | | | | | | | | | | |
| saG4 gRNA | | | | | | | | | | | | | |
| G1397 DddAtox-N- | G | 97.42% | 97.43% | 0.02% | 0.01% | 0.02% | 0.06% | 97.30% | 97.29% | 0.29% | 97.31% | 0.04% | 0.09% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 2.54% | 2.54% | 2.50% | 2.51% | 2.64% | 2.64% | 2.67% | 2.67% | 2.63% | 2.65% | 2.65% | 2.66% |
| SaKKH-Cas9 with | | | | | | | | | | | | | |
| saG4 gRNA | | | | | | | | | | | | | |
| G1397 DddAtox-C- | A | 0.01% | 0.02% | 0.01% | 0.01% | 0.02% | 99.46% | 0.01% | 0.01% | 0.03% | 0.01% | 99.43% | 99.37% |
| SaKKH-Cas9 with | | | | | | | | | | | | | |
| saG4 gRNA | | | | | | | | | | | | | |
| G1397 DddAtox-C- | T | 0.01% | 0.01% | 0.02% | 0.01% | 0.02% | 0.01% | 0.02% | 0.01% | 99.18% | 0.01% | 0.01% | 0.01% |
| SaKKH-Cas9 with | | | | | | | | | | | | | |
| saG4 gRNA | | | | | | | | | | | | | |
| G1397 DddAtox-C- | C | 0.00% | 0.00% | 99.63% | 99.64% | 99.51% | 0.04% | 0.00% | 0.00% | 0.05% | 0.00% | 0.06% | 0.03% |
| SaKKH-Cas9 with | | | | | | | | | | | | | |
| saG4 gRNA | | | | | | | | | | | | | |
| G1397 DddAtox-C- | G | 99.86% | 99.86% | 0.01% | 0.01% | 0.00% | 0.05% | 99.52% | 99.53% | 0.29% | 99.51% | 0.04% | 0.09% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.11% | 0.11% | 0.33% | 0.34% | 0.44% | 0.44% | 0.45% | 0.45% | 0.46% | 0.46% | 0.47% | 0.50% |
| SaKKH-Cas9 with | | | | | | | | | | | | | |
| saG4 gRNA | | | | | | | | | | | | | |
| Batch | Nucleotide | G | G | T | G | T | G | G | T | T | C | C | A |
| G1333 DddAtox-N- | A | 0.00% | 0.01% | 0.04% | 0.01% | 0.03% | 0.02% | 0.00% | 0.01% | 0.00% | 0.01% | 0.02% | 99.10% |
| SaKKH-Cas9 with | | | | | | | | | | | | | |
| saG4 gRNA | | | | | | | | | | | | | |
| G1333 DddAtox-N- | T | 0.01% | 0.01% | 98.73% | 0.02% | 99.02% | 0.01% | 0.01% | 99.06% | 99.06% | 0.06% | 0.02% | 0.01% |
| SaKKH-Cas9 with | | | | | | | | | | | | | |
| saG4 gRNA | | | | | | | | | | | | | |
| G1333 DddAtox-N- | C | 0.00% | 0.00% | 0.05% | 0.00% | 0.03% | 0.00% | 0.00% | 0.02% | 0.01% | 99.00% | 99.01% | 0.01% |
| SaKKH-Cas9 with | | | | | | | | | | | | | |
| saG4 gRNA | | | | | | | | | | | | | |
| G1333 DddAtox-N- | G | 99.20% | 99.14% | 0.27% | 99.11% | 0.06% | 99.11% | 99.12% | 0.03% | 0.00% | 0.02% | 0.00% | 0.04% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.78% | 0.84% | 0.86% | 0.86% | 0.86% | 0.86% | 0.87% | 0.89% | 0.92% | 0.92% | 0.94% | 0.84% |
| SaKKH-Cas9 with | | | | | | | | | | | | | |
| saG4 gRNA | | | | | | | | | | | | | |
| G1333 DddAtox-C- | A | 0.01% | 0.00% | 0.03% | 0.01% | 0.02% | 0.01% | 0.01% | 0.01% | 0.01% | 0.00% | 0.02% | 99.50% |
| SaKKH-Cas9 with | | | | | | | | | | | | | |
| saG4 gRNA | | | | | | | | | | | | | |
| G1333 DddAtox-C- | T | 0.01% | 0.02% | 99.23% | 0.02% | 99.48% | 0.01% | 0.00% | 99.50% | 99.51% | 0.03% | 0.01% | 0.01% |
| SaKKH-Cas9 with | | | | | | | | | | | | | |
| saG4 gRNA | | | | | | | | | | | | | |
| G1333 DddAtox-C- | C | 0.00% | 0.00% | 0.06% | 0.01% | 0.02% | 0.01% | 0.00% | 0.03% | 0.02% | 99.51% | 99.51% | 0.01% |
| SaKKH-Cas9 with | | | | | | | | | | | | | |
| saG4 gRNA | | | | | | | | | | | | | |
| G1333 DddAtox-C- | G | 99.65% | 99.63% | 0.31% | 99.58% | 0.08% | 99.56% | 99.56% | 0.02% | 0.00% | 0.00% | 0.00% | 0.03% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.33% | 0.35% | 0.36% | 0.38% | 0.40% | 0.42% | 0.42% | 0.45% | 0.46% | 0.45% | 0.46% | 0.45% |
| SaKKH-Cas9 with | | | | | | | | | | | | | |
| saG4 gRNA | | | | | | | | | | | | | |
| G1397 DddAtox-N- | A | 0.01% | 0.01% | 0.04% | 0.01% | 0.01% | 0.02% | 0.03% | 0.02% | 0.01% | 0.00% | 0.02% | 96.50% |
| SaKKH-Cas9 with | | | | | | | | | | | | | |
| saG4 gRNA | | | | | | | | | | | | | |
| G1397 DddAtox-N- | T | 0.01% | 0.01% | 96.22% | 0.02% | 96.47% | 0.01% | 0.01% | 96.46% | 96.51% | 0.04% | 0.01% | 0.01% |
| SaKKH-Cas9 with | | | | | | | | | | | | | |
| saG4 gRNA | | | | | | | | | | | | | |
| G1397 DddAtox-N- | C | 0.01% | 0.02% | 0.07% | 0.01% | 0.03% | 0.00% | 0.01% | 0.05% | 0.02% | 96.51% | 96.50% | 0.01% |
| SaKKH-Cas9 with | | | | | | | | | | | | | |
| saG4 gRNA | | | | | | | | | | | | | |
| G1397 DddAtox-N- | G | 97.32% | 96.55% | 0.26% | 96.56% | 0.08% | 96.54% | 96.52% | 0.02% | 0.01% | 0.00% | 0.01% | 0.04% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 2.66% | 3.41% | 3.41% | 3.41% | 3.42% | 3.43% | 3.44% | 3.45% | 3.45% | 3.45% | 3.45% | 3.45% |
| SaKKH-Cas9 with | | | | | | | | | | | | | |
| saG4 gRNA | | | | | | | | | | | | | |
| G1397 DddAtox-C- | A | 0.00% | 0.02% | 0.05% | 0.01% | 0.02% | 0.01% | 0.01% | 0.01% | 0.01% | 0.01% | 0.01% | 99.28% |
| SaKKH-Cas9 with | | | | | | | | | | | | | |
| saG4 gRNA | | | | | | | | | | | | | |
| G1397 DddAtox-C- | T | 0.02% | 0.03% | 99.08% | 0.01% | 99.32% | 0.02% | 0.01% | 99.32% | 99.34% | 0.03% | 0.02% | 0.01% |
| SaKKH-Cas9 with | | | | | | | | | | | | | |
| saG4 gRNA | | | | | | | | | | | | | |
| G1397 DddAtox-C- | C | 0.00% | 0.00% | 0.05% | 0.01% | 0.02% | 0.00% | 0.00% | 0.03% | 0.02% | 99.33% | 99.32% | 0.01% |
| SaKKH-Cas9 with | | | | | | | | | | | | | |
| saG4 gRNA | | | | | | | | | | | | | |
| G1397 DddAtox-C- | G | 99.47% | 99.42% | 0.27% | 99.42% | 0.06% | 99.38% | 99.40% | 0.02% | 0.01% | 0.01% | 0.00% | 0.05% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.51% | 0.53% | 0.56% | 0.56% | 0.58% | 0.59% | 0.58% | 0.61% | 0.63% | 0.63% | 0.64% | 0.65% |
| SaKKH-Cas9 with | | | | | | | | | | | | | |
| saG4 gRNA | | | | | | | | | | | | | |
| Batch | Nucleotide | G | A | A | C | C | G | G | A | G | G | A | C |
| G1333 DddAtox-N- | A | 0.01% | 99.11% | 99.11% | 0.01% | 0.01% | 0.02% | 0.01% | 98.94% | 0.01% | 0.01% | 99.12% | 0.00% |
| SaKKH-Cas9 with | | | | | | | | | | | | | |
| saG4 gRNA | | | | | | | | | | | | | |
| G1333 DddAtox-N- | T | 0.01% | 0.01% | 0.01% | 0.01% | 0.02% | 0.02% | 0.01% | 0.01% | 0.01% | 0.01% | 0.02% | 0.02% |
| SaKKH-Cas9 with | | | | | | | | | | | | | |
| saG4 gRNA | | | | | | | | | | | | | |
| G1333 DddAtox-N- | C | 0.00% | 0.02% | 0.02% | 99.02% | 99.00% | 0.00% | 0.00% | 0.02% | 0.00% | 0.01% | 0.04% | 99.28% |
| SaKKH-Cas9 with | | | | | | | | | | | | | |
| saG4 gRNA | | | | | | | | | | | | | |
| G1333 DddAtox-N- | G | 99.15% | 0.01% | 0.02% | 0.00% | 0.00% | 98.99% | 99.00% | 0.06% | 98.91% | 98.93% | 0.02% | 0.00% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.84% | 0.85% | 0.85% | 0.95% | 0.97% | 0.97% | 0.97% | 0.97% | 1.07% | 1.04% | 0.81% | 0.70% |
| SaKKH-Cas9 with | | | | | | | | | | | | | |
| saG4 gRNA | | | | | | | | | | | | | |
| G1333 DddAtox-C- | A | 0.01% | 99.50% | 99.50% | 0.00% | 0.00% | 0.02% | 0.01% | 99.41% | 0.01% | 0.01% | 99.60% | 0.01% |
| SaKKH-Cas9 with | | | | | | | | | | | | | |
| saG4 gRNA | | | | | | | | | | | | | |
| G1333 DddAtox-C- | T | 0.00% | 0.00% | 0.01% | 0.01% | 0.01% | 0.02% | 0.01% | 0.02% | 0.01% | 0.00% | 0.02% | 0.01% |
| SaKKH-Cas9 with | | | | | | | | | | | | | |
| saG4 gRNA | | | | | | | | | | | | | |
| G1333 DddAtox-C- | C | 0.00% | 0.02% | 0.02% | 99.52% | 99.50% | 0.00% | 0.00% | 0.02% | 0.00% | 0.00% | 0.05% | 99.75% |
| SaKKH-Cas9 with | | | | | | | | | | | | | |
| saG4 gRNA | | | | | | | | | | | | | |
| G1333 DddAtox-C- | G | 99.53% | 0.03% | 0.02% | 0.00% | 0.01% | 99.48% | 99.56% | 0.06% | 99.41% | 99.42% | 0.03% | 0.00% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.45% | 0.45% | 0.46% | 0.46% | 0.48% | 0.48% | 0.48% | 0.50% | 0.57% | 0.56% | 0.31% | 0.22% |
| SaKKH-Cas9 with | | | | | | | | | | | | | |
| saG4 gRNA | | | | | | | | | | | | | |
| G1397 DddAtox-N- | A | 0.01% | 96.51% | 96.49% | 0.02% | 0.02% | 0.02% | 0.01% | 96.43% | 0.01% | 0.01% | 98.49% | 0.01% |
| SaKKH-Cas9 with | | | | | | | | | | | | | |
| saG4 gRNA | | | | | | | | | | | | | |
| G1397 DddAtox-N- | T | 0.01% | 0.02% | 0.00% | 0.00% | 0.01% | 0.01% | 0.01% | 0.03% | 0.01% | 0.02% | 0.01% | 0.01% |
| SaKKH-Cas9 with | | | | | | | | | | | | | |
| saG4 gRNA | | | | | | | | | | | | | |
| G1397 DddAtox-N- | C | 0.00% | 0.01% | 0.03% | 96.51% | 96.51% | 0.00% | 0.00% | 0.03% | 0.00% | 0.01% | 0.09% | 98.65% |
| SaKKH-Cas9 with | | | | | | | | | | | | | |
| saG4 gRNA | | | | | | | | | | | | | |
| G1397 DddAtox-N- | G | 96.53% | 0.02% | 0.04% | 0.02% | 0.01% | 96.51% | 96.51% | 0.06% | 96.48% | 96.48% | 0.04% | 0.01% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 3.45% | 3.44% | 3.44% | 3.44% | 3.45% | 3.45% | 3.47% | 3.45% | 3.51% | 3.49% | 1.37% | 1.31% |
| SaKKH-Cas9 with | | | | | | | | | | | | | |
| saG4 gRNA | | | | | | | | | | | | | |
| G1397 DddAtox-C- | A | 0.01% | 99.28% | 99.25% | 0.01% | 0.01% | 0.01% | 0.02% | 99.31% | 0.01% | 0.01% | 99.37% | 0.01% |
| SaKKH-Cas9 with | | | | | | | | | | | | | |
| saG4 gRNA | | | | | | | | | | | | | |
| G1397 DddAtox-C- | T | 0.01% | 0.01% | 0.02% | 0.01% | 0.03% | 0.02% | 0.01% | 0.01% | 0.02% | 0.02% | 0.01% | 0.01% |
| SaKKH-Cas9 with | | | | | | | | | | | | | |
| saG4 gRNA | | | | | | | | | | | | | |
| G1397 DddAtox-C- | C | 0.01% | 0.02% | 0.04% | 99.30% | 99.26% | 0.00% | 0.00% | 0.03% | 0.00% | 0.01% | 0.05% | 99.60% |
| SaKKH-Cas9 with | | | | | | | | | | | | | |
| saG4 gRNA | | | | | | | | | | | | | |
| G1397 DddAtox-C- | G | 99.31% | 0.03% | 0.02% | 0.01% | 0.01% | 99.28% | 99.28% | 0.05% | 99.25% | 99.26% | 0.02% | 0.00% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.66% | 0.66% | 0.68% | 0.68% | 0.69% | 0.69% | 0.69% | 0.59% | 0.72% | 0.70% | 0.55% | 0.37% |
| SaKKH-Cas9 with | | | | | | | | | | | | | |
| saG4 gRNA | | | | | | | | | | | | | |
| Batch | Nucleotide | A | A | A | G | T | A | C | A | A | A | C | G |
| G1333 DddAtox-N- | A | 99.27% | 99.27% | 99.26% | 0.01% | 0.11 | 99.68% | 0.02% | 99.71% | 99.72% | 99.72% | 0.01% | 0.03% |
| SaKKH-Cas9 with | | | | | | | | | | | | | |
| saG4 gRNA | | | | | | | | | | | | | |
| G1333 DddAtox-N- | T | 0.01% | 0.02% | 0.00% | 0.01% | 99.27% | 0.00% | 0.02% | 0.00% | 0.00% | 0.00% | 0.02% | 0.00% |
| SaKKH-Cas9 with | | | | | | | | | | | | | |
| saG4 gRNA | | | | | | | | | | | | | |
| G1333 DddAtox-N- | C | 0.02% | 0.03% | 0.03% | 0.00% | 0.05% | 0.01% | 99.68% | 0.02% | 0.02% | 0.02% | 99.72% | 0.01% |
| SaKKH-Cas9 with | | | | | | | | | | | | | |
| saG4 gRNA | | | | | | | | | | | | | |
| G1333 DddAtox-N- | G | 0.03% | 0.01% | 0.03% | 99.41% | 0.07% | 0.02% | 0.00% | 0.01% | 0.01% | 0.01% | 0.01% | 99.96% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.68% | 0.68% | 0.67% | 0.57% | 0.50% | 0.28% | 0.27% | 0.26% | 0.25% | 0.25% | 0.25% | 0.00% |
| SaKKH-Cas9 with | | | | | | | | | | | | | |
| saG4 gRNA | | | | | | | | | | | | | |
| G1333 DddAtox-C- | A | 99.74% | 99.75% | 99.75% | 0.01% | 0.14% | 99.88% | 0.02% | 99.90% | 99.89% | 99.89% | 0.01% | 0.03% |
| SaKKH-Cas9 with | | | | | | | | | | | | | |
| saG4 gRNA | | | | | | | | | | | | | |
| G1333 DddAtox-C- | T | 0.01% | 0.01% | 0.01% | 0.00% | 99.63% | 0.02% | 0.00% | 0.00% | 0.00% | 0.00% | 0.02% | 0.01% |
| SaKKH-Cas9 with | | | | | | | | | | | | | |
| saG4 gRNA | | | | | | | | | | | | | |
| G1333 DddAtox-C- | C | 0.02% | 0.03% | 0.04% | 0.00% | 0.05% | 0.01% | 99.90% | 0.02% | 0.03% | 0.02% | 99.90% | 0.01% |
| SaKKH-Cas9 with | | | | | | | | | | | | | |
| saG4 gRNA | | | | | | | | | | | | | |
| G1333 DddAtox-C- | G | 0.02% | 0.01% | 0.05% | 99.84% | 0.07% | 0.01% | 0.00% | 0.01% | 0.01% | 0.02% | 0.01% | 99.91% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.21% | 0.21% | 0.15% | 0.14% | 0.12% | 0.08% | 0.07% | 0.07% | 0.06% | 0.07% | 0.07% | 0.05% |
| SaKKH-Cas9 with | | | | | | | | | | | | | |
| saG4 gRNA | | | | | | | | | | | | | |
| G1397 DddAtox-N- | A | 98.75% | 98.71% | 98.73% | 0.01% | 0.12% | 98.80% | 0.02% | 98.84% | 99.19% | 99.19% | 0.01% | 0.03% |
| SaKKH-Cas9 with | | | | | | | | | | | | | |
| saG4 gRNA | | | | | | | | | | | | | |
| G1397 DddAtox-N- | T | 0.02% | 0.03% | 0.00% | 0.02% | 98.44% | 0.02% | 0.01% | 0.00% | 0.02% | 0.01% | 0.05% | 0.01% |
| SaKKH-Cas9 with | | | | | | | | | | | | | |
| saG4 gRNA | | | | | | | | | | | | | |
| G1397 DddAtox-N- | C | 0.02% | 0.05% | 0.06% | 0.00% | 0.23% | 0.03% | 98.85% | 0.02% | 0.03% | 0.03% | 99.20% | 0.02% |
| SaKKH-Cas9 with | | | | | | | | | | | | | |
| saG4 gRNA | | | | | | | | | | | | | |
| G1397 DddAtox-N- | G | 0.03% | 0.03% | 0.04% | 98.80% | 0.06% | 0.03% | 0.00% | 0.04% | 0.01% | 0.03% | 0.00% | 99.20% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 1.17% | 1.17% | 1.16% | 1.16% | 1.15% | 1.12% | 1.11% | 1.10% | 0.75% | 0.75% | 0.74% | 0.74% |
| SaKKH-Cas9 with | | | | | | | | | | | | | |
| saG4 gRNA | | | | | | | | | | | | | |
| G1397 DddAtox-C- | A | 99.69% | 99.69% | 99.65% | 0.01% | 0.13% | 99.91% | 0.02% | 99.93% | 99.94% | 99.93% | 0.02% | 0.03% |
| SaKKH-Cas9 with | | | | | | | | | | | | | |
| saG4 gRNA | | | | | | | | | | | | | |
| G1397 DddAtox-C- | T | 0.01% | 0.01% | 0.01% | 0.01% | 99.66% | 0.01% | 0.01% | 0.00% | 0.00% | 0.00% | 0.02% | 0.01% |
| SaKKH-Cas9 with | | | | | | | | | | | | | |
| saG4 gRNA | | | | | | | | | | | | | |
| G1397 DddAtox-C- | C | 0.02% | 0.03% | 0.04% | 0.00% | 0.05% | 0.02% | 99.92% | 0.02% | 0.03% | 0.03% | 99.93% | 0.00% |
| SaKKH-Cas9 with | | | | | | | | | | | | | |
| saG4 gRNA | | | | | | | | | | | | | |
| G1397 DddAtox-C- | G | 0.02% | 0.01% | 0.05% | 99.73% | 0.06% | 0.02% | 0.00% | 0.01% | 0.01% | 0.02% | 0.01% | 99.94% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.27% | 0.26% | 0.26% | 0.25% | 0.10% | 0.05% | 0.05% | 0.03% | 0.02% | 0.02% | 0.02% | 0.02% |
| SaKKH-Cas9 with | | | | | | | | | | | | | |
| saG4 gRNA | | | | | | | | | | | | | |
| Batch | Nucleotide | G | C | A | G | A | A | G | C | T | G | G | A |
| G1333 DddAtox-N- | A | 0.03% | 0.01% | 99.92% | 0.00% | 99.95% | 99.85% | 0.00% | 0.00% | 0.00% | 0.01% | 0.01% | 99.86% |
| SaKKH-Cas9 with | | | | | | | | | | | | | |
| saG4 gRNA | | | | | | | | | | | | | |
| G1333 DddAtox-N- | T | 0.01% | 0.01% | 0.01% | 0.01% | 0.00% | 0.00% | 0.01% | 0.01% | 99.95% | 0.02% | 0.02% | 0.01% |
| SaKKH-Cas9 with | | | | | | | | | | | | | |
| saG4 gRNA | | | | | | | | | | | | | |
| G1333 DddAtox-N- | C | 0.00% | 99.96% | 0.01% | 0.00% | 0.03% | 0.05% | 0.00% | 99.97% | 0.02% | 0.01% | 0.00% | 0.03% |
| SaKKH-Cas9 with | | | | | | | | | | | | | |
| saG4 gRNA | | | | | | | | | | | | | |
| G1333 DddAtox-N- | G | 99.96% | 0.01% | 0.06% | 99.99% | 0.02% | 0.09% | 99.98% | 0.01% | 0.02% | 99.97% | 99.97% | 0.10% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | A | 0.03% | 0.02% | 99.87% | 0.01% | 99.93% | 99.83% | 0.00% | 0.00% | 0.00% | 0.01% | 0.01% | 99.81% |
| SaKKH-Cas9 with | | | | | | | | | | | | | |
| saG4 gRNA | | | | | | | | | | | | | |
| G1333 DddAtox-C- | T | 0.00% | 0.01% | 0.01% | 0.00% | 0.01% | 0.00% | 0.00% | 0.01% | 99.92% | 0.01% | 0.01% | 0.02% |
| SaKKH-Cas9 with | | | | | | | | | | | | | |
| saG4 gRNA | | | | | | | | | | | | | |
| G1333 DddAtox-C- | C | 0.00% | 99.91% | 0.03% | 0.00% | 0.02% | 0.04% | 0.00% | 99.97% | 0.03% | 0.00% | 0.00% | 0.05% |
| SaKKH-Cas9 with | | | | | | | | | | | | | |
| saG4 gRNA | | | | | | | | | | | | | |
| G1333 DddAtox-C- | G | 99.92% | 0.01% | 0.05% | 99.94% | 0.01% | 0.11% | 99.97% | 0.00% | 0.03% | 99.96% | 99.95% | 0.11% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.05% | 0.05% | 0.05% | 0.05% | 0.02% | 0.02% | 0.02% | 0.02% | 0.02% | 0.02% | 0.02% | 0.02% |
| SaKKH-Cas9 with | | | | | | | | | | | | | |
| saG4 gRNA | | | | | | | | | | | | | |
| G1397 DddAtox-N- | A | 0.02% | 0.02% | 99.12% | 0.01% | 99.16% | 99.07% | 0.01% | 0.01% | 0.00% | 0.00% | 0.01% | 99.76% |
| SaKKH-Cas9 with | | | | | | | | | | | | | |
| saG4 gRNA | | | | | | | | | | | | | |
| G1397 DddAtox-N- | T | 0.01% | 0.02% | 0.03% | 0.03% | 0.03% | 0.02% | 0.01% | 0.02% | 99.95% | 0.02% | 0.02% | 0.02% |
| SaKKH-Cas9 with | | | | | | | | | | | | | |
| saG4 gRNA | | | | | | | | | | | | | |
| G1397 DddAtox-N- | C | 0.01% | 99.21% | 0.05% | 0.00% | 0.05% | 0.06% | 0.00% | 99.96% | 0.03% | 0.01% | 0.01% | 0.11% |
| SaKKH-Cas9 with | | | | | | | | | | | | | |
| saG4 gRNA | | | | | | | | | | | | | |
| G1397 DddAtox-N- | G | 99.21% | 0.01% | 0.07% | 99.23% | 0.02% | 0.11% | 99.24% | 0.01% | 0.02% | 99.97% | 99.95% | 0.11% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 0.74% | 0.74% | 0.73% | 0.73% | 0.73% | 0.74% | 0.73% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | A | 0.03% | 0.01% | 99.90% | 0.01% | 99.93% | 99.86% | 0.00% | 0.00% | 0.01% | 0.01% | 0.00% | 99.82% |
| SaKKH-Cas9 with | | | | | | | | | | | | | |
| saG4 gRNA | | | | | | | | | | | | | |
| G1397 DddAtox-C- | T | 0.01% | 0.01% | 0.01% | 0.01% | 0.01% | 0.01% | 0.01% | 0.01% | 99.93% | 0.01% | 0.02% | 0.01% |
| SaKKH-Cas9 with | | | | | | | | | | | | | |
| saG4 gRNA | | | | | | | | | | | | | |
| G1397 DddAtox-C- | C | 0.00% | 99.96% | 0.03% | 0.00% | 0.03% | 0.05% | 0.00% | 99.98% | 0.04% | 0.00% | 0.00% | 0.04% |
| SaKKH-Cas9 with | | | | | | | | | | | | | |
| saG4 gRNA | | | | | | | | | | | | | |
| G1397 DddAtox-C- | G | 99.93% | 0.01% | 0.06% | 99.98% | 0.03% | 0.09% | 99.98% | 0.01% | 0.03% | 99.97% | 99.97% | 0.13% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.02% | 0.01% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| Batch | Nucleotide | G | G | A | G | G | A | A | G | G | G | C | C |
| G1333 DddAtox-N- | A | 0.00% | 0.01% | 99.88% | 0.01% | 0.01% | 99.92% | 99.91% | 0.02% | 0.01% | 0.01% | 0.01% | 0.02% |
| SaKKH-Cas9 with | | | | | | | | | | | | | |
| saG4 gRNA | | | | | | | | | | | | | |
| G1333 DddAtox-N- | T | 0.02% | 0.02% | 0.01% | 0.02% | 0.01% | 0.01% | 0.01% | 0.01% | 0.01% | 0.02% | 0.04% | 0.08% |
| SaKKH-Cas9 with | | | | | | | | | | | | | |
| saG4 gRNA | | | | | | | | | | | | | |
| G1333 DddAtox-N- | C | 0.00% | 0.00% | 0.04% | 0.00% | 0.01% | 0.02% | 0.02% | 0.01% | 0.01% | 0.00% | 99.93% | 99.88% |
| SaKKH-Cas9 with | | | | | | | | | | | | | |
| saG4 gRNA | | | | | | | | | | | | | |
| G1333 DddAtox-N- | G | 99.97% | 99.97% | 0.07% | 99.97% | 99.97% | 0.06% | 0.06% | 99.97% | 99.97% | 99.97% | 0.02% | 0.01% |
| SaKKH-Cas9 with | | | | | | | | | | | | | |
| saG4 gRNA | | | | | | | | | | | | | |
| G1333 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-N- | — | 0.00% | 0.00% | 0.00% | 0.00% | 0.00% | 0.00% | 0.00% | 0.00% | 0.00% | 0.00% | 0.00% | 0.01% |
| SaKKH-Cas9 with | | | | | | | | | | | | | |
| saG4 gRNA | | | | | | | | | | | | | |
| G1333 DddAtox-C- | A | 0.01% | 0.01% | 99.85% | 0.00% | 0.02% | 99.90% | 99.87% | 0.01% | 0.00% | 0.00% | 0.02% | 0.02% |
| SaKKH-Cas9 with | | | | | | | | | | | | | |
| saG4 gRNA | | | | | | | | | | | | | |
| G1333 DddAtox-C- | T | 0.02% | 0.01% | 0.01% | 0.01% | 0.01% | 0.01% | 0.01% | 0.01% | 0.00% | 0.00% | 0.05% | 0.10% |
| SaKKH-Cas9 with | | | | | | | | | | | | | |
| saG4 gRNA | | | | | | | | | | | | | |
| G1333 DddAtox-C- | C | 0.00% | 0.00% | 0.02% | 0.00% | 0.00% | 0.02% | 0.02% | 0.01% | 0.00% | 0.01% | 99.90% | 99.85% |
| SaKKH-Cas9 with | | | | | | | | | | | | | |
| saG4 gRNA | | | | | | | | | | | | | |
| G1333 DddAtox-C- | G | 99.95% | 99.96% | 0.09% | 99.97% | 99.95% | 0.05% | 0.09% | 99.95% | 99.98% | 99.96% | 0.03% | 0.03% |
| SaKKH-Cas9 with | | | | | | | | | | | | | |
| saG4 gRNA | | | | | | | | | | | | | |
| G1333 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1333 DddAtox-C- | — | 0.02% | 0.02% | 0.02% | 0.02% | 0.02% | 0.02% | 0.02% | 0.02% | 0.02% | 0.02% | 0.00% | 0.00% |
| SaKKH-Cas9 with | | | | | | | | | | | | | |
| saG4 gRNA | | | | | | | | | | | | | |
| G1397 DddAtox-N- | A | 0.03% | 0.01% | 99.83% | 0.03% | 0.03% | 99.89% | 99.88% | 0.04% | 0.02% | 0.01% | 0.01% | 0.02% |
| SaKKH-Cas9 with | | | | | | | | | | | | | |
| saG4 gRNA | | | | | | | | | | | | | |
| G1397 DddAtox-N- | T | 0.02% | 0.01% | 0.03% | 0.00% | 0.01% | 0.02% | 0.01% | 0.01% | 0.01% | 0.01% | 0.05% | 0.09% |
| SaKKH-Cas9 with | | | | | | | | | | | | | |
| saG4 gRNA | | | | | | | | | | | | | |
| G1397 DddAtox-N- | C | 0.00% | 0.00% | 0.05% | 0.01% | 0.02% | 0.04% | 0.03% | 0.01% | 0.02% | 0.00% | 99.92% | 99.87% |
| SaKKH-Cas9 with | | | | | | | | | | | | | |
| saG4 gRNA | | | | | | | | | | | | | |
| G1397 DddAtox-N- | G | 99.95% | 99.97% | 0.09% | 99.96% | 99.94% | 0.05% | 0.07% | 99.94% | 99.95% | 99.98% | 0.03% | 0.02% |
| SaKKH-Cas9 with | | | | | | | | | | | | | |
| saG4 gRNA | | | | | | | | | | | | | |
| G1397 DddAtox-N- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-N- | — | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | A | 0.00% | 0.01% | 99.84% | 0.01% | 0.01% | 99.91% | 99.88% | 0.01% | 0.01% | 0.01% | 0.01% | 0.04% |
| SaKKH-Cas9 with | | | | | | | | | | | | | |
| saG4 gRNA | | | | | | | | | | | | | |
| G1397 DddAtox-C- | T | 0.02% | 0.01% | 0.02% | 0.02% | 0.01% | 0.01% | 0.00% | 0.01% | 0.01% | 0.02% | 0.03% | 0.09% |
| SaKKH-Cas9 with | | | | | | | | | | | | | |
| saG4 gRNA | | | | | | | | | | | | | |
| G1397 DddAtox-C- | C | 0.00% | 0.00% | 0.04% | 0.00% | 0.00% | 0.02% | 0.02% | 0.01% | 0.00% | 0.00% | 99.92% | 99.85% |
| SaKKH-Cas9 with | | | | | | | | | | | | | |
| saG4 gRNA | | | | | | | | | | | | | |
| G1397 DddAtox-C- | G | 99.98% | 99.97% | 0.10% | 99.97% | 99.97% | 0.06% | 0.09% | 99.98% | 99.98% | 99.97% | 0.04% | 0.02% |
| SaKKH-Cas9 with | | | | | | | | | | | | | |
| saG4 gRNA | | | | | | | | | | | | | |
| G1397 DddAtox-C- | N | 0.00% | 0.00% | 0.00% | 0.00% | 0.00% | 0.00% | 0.00% | 0.00% | 0.00% | 0.00% | 0.00% | 0.00% |
| SaKKH-Cas9 with | | | | | | | | | | | | | |
| saG4 gRNA | | | | | | | | | | | | | |
| G1397 DddAtox-C- | — | 0.00% | 0.00% | 0.00% | 0.01% | 0.01% | 0.01% | 0.01% | 0.00% | 0.00% | 0.00% | 0.00% | 0.00% |
| SaKKH-Cas9 with | | | | | | | | | | | | | |
| saG4 gRNA | | | | | | | | | | | | | |

| Batch | Nucleotide | T | G | A | G | T | C | C | G |
|---|---|---|---|---|---|---|---|---|---|
| G1333 DddAtox-N- | A | 0.01% | 0.01% | 99.88% | 0.01% | 0.02% | 0.02% | 0.01% | 0.02% |
| SaKKH-Cas9 with | | | | | | | | | |
| saG4 gRNA | | | | | | | | | |
| G1333 DddAtox-N- | T | 99.91% | 0.01% | 0.00% | 0.01% | 99.80% | 0.03% | 0.04% | 0.01% |
| SaKKH-Cas9 with | | | | | | | | | |
| saG4 gRNA | | | | | | | | | |
| G1333 DddAtox-N- | C | 0.04% | 0.01% | 0.04% | 0.00% | 0.09% | 99.95% | 99.87% | 0.00% |
| SaKKH-Cas9 with | | | | | | | | | |
| saG4 gRNA | | | | | | | | | |
| G1333 DddAtox-N- | G | 0.03% | 99.97% | 0.08% | 99.97% | 0.06% | 0.01% | 0.01% | 99.92% |
| SaKKH-Cas9 with | | | | | | | | | |
| saG4 gRNA | | | | | | | | | |
| G1333 DddAtox-N- | N | 0.00% | 0.00% | 0.00% | 0.00% | 0.00% | 0.00% | 0.00% | 0.00% |
| SaKKH-Cas9 with | | | | | | | | | |
| saG4 gRNA | | | | | | | | | |
| G1333 DddAtox-N- | — | 0.00% | 0.00% | 0.00% | 0.01% | 0.03% | 0.00% | 0.07% | 0.04% |
| SaKKH-Cas9 with | | | | | | | | | |
| saG4 gRNA | | | | | | | | | |
| G1333 DddAtox-C- | A | 0.01% | 0.01% | 99.86% | 0.01% | 0.03% | 0.01% | 0.01% | 0.02% |
| SaKKH-Cas9 with | | | | | | | | | |
| saG4 gRNA | | | | | | | | | |
| G1333 DddAtox-C- | T | 99.89% | 0.02% | 0.01% | 0.01% | 99.79% | 0.04% | 0.05% | 0.00% |
| SaKKH-Cas9 with | | | | | | | | | |
| saG4 gRNA | | | | | | | | | |
| G1333 DddAtox-C- | C | 0.04% | 0.00% | 0.04% | 0.00% | 0.07% | 99.94% | 99.84% | 0.01% |
| SaKKH-Cas9 with | | | | | | | | | |
| saG4 gRNA | | | | | | | | | |
| G1333 DddAtox-C- | G | 0.05% | 99.97% | 0.09% | 99.95% | 0.07% | 0.01% | 0.03% | 99.91% |
| SaKKH-Cas9 with | | | | | | | | | |
| saG4 gRNA | | | | | | | | | |
| G1333 DddAtox-C- | N | 0.00% | 0.00% | 0.00% | 0.00% | 0.00% | 0.00% | 0.00% | 0.00% |
| SaKKH-Cas9 with | | | | | | | | | |
| saG4 gRNA | | | | | | | | | |
| G1333 DddAtox-C- | — | 0.00% | 0.00% | 0.00% | 0.03% | 0.04% | 0.00% | 0.08% | 0.06% |
| SaKKH-Cas9 with | | | | | | | | | |
| saG4 gRNA | | | | | | | | | |
| G1397 DddAtox-N- | A | 0.02% | 0.03% | 99.83% | 0.02% | 0.05% | 0.02% | 0.01% | 0.03% |
| SaKKH-Cas9 with | | | | | | | | | |
| saG4 gRNA | | | | | | | | | |
| G1397 DddAtox-N- | T | 99.89% | 0.01% | 0.01% | 0.00% | 99.75% | 0.05% | 0.06% | 0.01% |
| SaKKH-Cas9 with | | | | | | | | | |
| saG4 gRNA | | | | | | | | | |
| G1397 DddAtox-N- | C | 0.05% | 0.00% | 0.06% | 0.01% | 0.07% | 99.92% | 99.83% | 0.01% |
| SaKKH-Cas9 with | | | | | | | | | |
| saG4 gRNA | | | | | | | | | |
| G1397 DddAtox-N- | G | 0.04% | 99.96% | 0.09% | 99.95% | 0.08% | 0.01% | 0.01% | 99.89% |
| SaKKH-Cas9 with | | | | | | | | | |
| saG4 gRNA | | | | | | | | | |
| G1397 DddAtox-N- | N | 0.00% | 0.00% | 0.00% | 0.00% | 0.00% | 0.00% | 0.00% | 0.00% |
| SaKKH-Cas9 with | | | | | | | | | |
| saG4 gRNA | | | | | | | | | |
| G1397 DddAtox-N- | — | 0.00% | 0.00% | 0.00% | 0.02% | 0.05% | 0.00% | 0.09% | 0.07% |
| SaKKH-Cas9 with | | | | | | | | | |
| saG4 gRNA | | | | | | | | | |
| G1397 DddAtox-C- | A | 0.01% | 0.02% | 99.86% | 0.00% | 0.02% | 0.01% | 0.01% | 0.01% |
| SaKKH-Cas9 with | | | | | | | | | |
| saG4 gRNA | | | | | | | | | |
| G1397 DddAtox-C- | T | 99.90% | 0.02% | 0.01% | 0.01% | 99.79% | 0.03% | 0.05% | 0.01% |
| SaKKH-Cas9 with | | | | | | | | | |
| saG4 gRNA | | | | | | | | | |
| G1397 DddAtox-C- | C | 0.05% | 0.00% | 0.03% | 0.00% | 0.08% | 99.95% | 99.83% | 0.00% |
| SaKKH-Cas9 with | | | | | | | | | |
| saG4 gRNA | | | | | | | | | |
| G1397 DddAtox-C- | G | 0.04% | 99.97% | 0.09% | 99.96% | 0.07% | 0.01% | 0.02% | 99.90% |
| SaKKH-Cas9 with | | | | | | | | | |
| saG4 gRNA | | | | | | | | | |
| G1397 DddAtox-C- | N | 0.00% | 0.00% | 0.00% | 0.00% | 0.00% | 0.00% | 0.00% | 0.00% |
| SaKKH-Cas9 with | | | | | | | | | |
| saG4 gRNA | | | | | | | | | |
| G1397 DddAtox-C- | — | 0.00% | 0.00% | 0.00% | 0.02% | 0.04% | 0.00% | 0.09% | 0.07% |
| SaKKH-Cas9 with | | | | | | | | | |
| saG4 gRNA | | | | | | | | | |

| Batch | Nucleotide | A | G | C | A | G | A | A | G |
|---|---|---|---|---|---|---|---|---|---|
| G1333 DddAtox-N- | A | 99.85% | 0.01% | 0.04% | 3.03% | 0.08% | 2.92% | 1.01% | 0.00% |
| SaKKH-Cas9 with | | | | | | | | | |
| saG4 gRNA | | | | | | | | | |
| G1333 DddAtox-N- | T | 0.01% | 0.01% | 0.03% | 0.01% | 0.01% | 0.00% | 0.00% | 0.00% |
| SaKKH-Cas9 with | | | | | | | | | |
| saG4 gRNA | | | | | | | | | |
| G1333 DddAtox-N- | C | 0.02% | 0.01% | 99.89% | 0.05% | 0.00% | 0.02% | 0.00% | 0.00% |
| SaKKH-Cas9 with | | | | | | | | | |
| saG4 gRNA | | | | | | | | | |
| G1333 DddAtox-N- | G | 0.09% | 99.94% | 0.02% | 0.11% | 3.07% | 0.05% | 0.01% | 1.01% |
| SaKKH-Cas9 with | | | | | | | | | |
| saG4 gRNA | | | | | | | | | |
| G1333 DddAtox-N- | N | 0.00% | 0.00% | 0.00% | 0.00% | 0.00% | 0.00% | 0.00% | 0.00% |
| SaKKH-Cas9 with | | | | | | | | | |
| saG4 gRNA | | | | | | | | | |
| G1333 DddAtox-N- | — | 0.03% | 0.04% | 0.02% | 96.81% | 96.84% | 97.01% | 98.98% | 98.98% |
| SaKKH-Cas9 with | | | | | | | | | |
| saG4 gRNA | | | | | | | | | |
| G1333 DddAtox-C- | A | 99.85% | 0.01% | 0.03% | 2.89% | 0.10% | 2.75% | 0.74% | 0.00% |
| SaKKH-Cas9 with | | | | | | | | | |
| saG4 gRNA | | | | | | | | | |
| G1333 DddAtox-C- | T | 0.01% | 0.01% | 0.04% | 0.02% | 0.01% | 0.01% | 0.00% | 0.00% |
| SaKKH-Cas9 with | | | | | | | | | |
| saG4 gRNA | | | | | | | | | |
| G1333 DddAtox-C- | C | 0.02% | 0.01% | 99.87% | 0.04% | 0.00% | 0.03% | 0.01% | 0.00% |
| SaKKH-Cas9 with | | | | | | | | | |
| saG4 gRNA | | | | | | | | | |
| G1333 DddAtox-C- | G | 0.09% | 99.92% | 0.02% | 0.10% | 2.93% | 0.05% | 0.00% | 0.74% |
| SaKKH-Cas9 with | | | | | | | | | |
| saG4 gRNA | | | | | | | | | |
| G1333 DddAtox-C- | N | 0.00% | 0.00% | 0.00% | 0.00% | 0.00% | 0.00% | 0.00% | 0.00% |
| SaKKH-Cas9 with | | | | | | | | | |
| saG4 gRNA | | | | | | | | | |
| G1333 DddAtox-C- | — | 0.03% | 0.05% | 0.03% | 96.95% | 96.96% | 97.17% | 99.25% | 99.26% |
| SaKKH-Cas9 with | | | | | | | | | |
| saG4 gRNA | | | | | | | | | |
| G1397 DddAtox-N- | A | 99.81% | 0.01% | 0.03% | 6.28% | 0.10% | 6.13% | 4.18% | 0.00% |
| SaKKH-Cas9 with | | | | | | | | | |
| saG4 gRNA | | | | | | | | | |
| G1397 DddAtox-N- | T | 0.01% | 0.02% | 0.04% | 0.01% | 0.01% | 0.01% | 0.00% | 0.00% |
| SaKKH-Cas9 with | | | | | | | | | |
| saG4 gRNA | | | | | | | | | |
| G1397 DddAtox-N- | C | 0.03% | 0.01% | 99.88% | 0.06% | 0.01% | 0.02% | 0.01% | 0.00% |
| SaKKH-Cas9 with | | | | | | | | | |
| saG4 gRNA | | | | | | | | | |
| G1397 DddAtox-N- | G | 0.12% | 99.92% | 0.01% | 0.09% | 6.31% | 0.08% | 0.01% | 4.19% |
| SaKKH-Cas9 with | | | | | | | | | |
| saG4 gRNA | | | | | | | | | |
| G1397 DddAtox-N- | N | 0.00% | 0.00% | 0.00% | 0.00% | 0.00% | 0.00% | 0.00% | 0.00% |
| SaKKH-Cas9 with | | | | | | | | | |
| saG4 gRNA | | | | | | | | | |
| G1397 DddAtox-N- | — | 0.03% | 0.04% | 0.04% | 93.55% | 93.57% | 93.76% | 95.80% | 95.81% |
| SaKKH-Cas9 with | | | | | | | | | |
| saG4 gRNA | | | | | | | | | |
| G1397 DddAtox-C- | A | 99.84% | 0.01% | 0.03% | 3.12% | 0.11% | 2.98% | 0.75% | 0.00% |
| SaKKH-Cas9 with | | | | | | | | | |
| saG4 gRNA | | | | | | | | | |
| G1397 DddAtox-C- | T | 0.01% | 0.00% | 0.03% | 0.01% | 0.01% | 0.01% | 0.00% | 0.00% |
| SaKKH-Cas9 with | | | | | | | | | |
| saG4 gRNA | | | | | | | | | |
| G1397 DddAtox-C- | C | 0.04% | 0.01% | 99.88% | 0.03% | 0.00% | 0.01% | 0.00% | 0.00% |
| SaKKH-Cas9 with | | | | | | | | | |
| saG4 gRNA | | | | | | | | | |
| G1397 DddAtox-C- | G | 0.08% | 99.91% | 0.03% | 0.12% | 3.14% | 0.06% | 0.00% | 0.76% |
| SaKKH-Cas9 with | | | | | | | | | |
| saG4 gRNA | | | | | | | | | |
| G1397 DddAtox-C- | N | 0.00% | 0.00% | 0.00% | 0.00% | 0.00% | 0.00% | 0.00% | 0.00% |
| SaKKH-Cas9 with | | | | | | | | | |
| saG4 gRNA | | | | | | | | | |
| G1397 DddAtox-C- | — | 0.03% | 0.06% | 0.03% | 96.72% | 96.74% | 96.94% | 99.24% | 99.24% |
| SaKKH-Cas9 with | | | | | | | | | |
| saG4 gRNA | | | | | | | | | |

TABLE 10B
dSpCas9 half with spG4 gRNA

| | Nucle- | | | | | | | | | | | | |
|---|---|---|---|---|---|---|---|---|---|---|---|---|---|
| Batch | otide | G | G | C | C | C | C | T | A | A | C | C | C |
| G1333 | A | 0.18% | 0.03% | 0.00% | 0.00% | 0.00% | 0.00% | 0.01% | 99.93% | 99.67% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | T | 0.20% | 0.03% | 0.01% | 0.00% | 0.01% | 0.00% | 99.93% | 0.00% | 0.00% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | C | 0.10% | 0.02% | 99.34% | 99.99% | 99.98% | 99.98% | 0.03% | 0.04% | 0.01% | 99.98% | 99.99% | 99.78% |
| DddAtox-N- | | | | | | | | | | | | | |
| dSpCas9 | | | | | | | | | | | | | |
| with spG4 gRNA | | | | | | | | | | | | | |
| G1333 | G | 99.51% | 99.92% | 0.65% | 0.01% | 0.00% | 0.00% | 0.00% | 0.02% | 0.01% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.01% | 0.01% | 0.03% | 0.00% | 0.30% | 0.01% | 0.00% | 0.20% |
| DddAtox-N- | | | | | | | | | | | | | |
| dSpCas9 | | | | | | | | | | | | | |
| with spG4 gRNA | | | | | | | | | | | | | |
| G1333 | A | 0.21% | 0.02% | 0.00% | 0.00% | 0.01% | 0.01% | 0.01% | 99.92% | 99.65% | 0.01% | 0.00% | 0.01% |
| DddAtox-C- | | | | | | | | | | | | | |
| dSpCas9 | | | | | | | | | | | | | |
| with spG4 gRNA | | | | | | | | | | | | | |
| G1333 | T | 0.22% | 0.03% | 0.00% | 0.01% | 0.00% | 0.00% | 99.92% | 0.00% | 0.00% | 0.00% | 0.01% | 0.00% |
| DddAtox-C- | | | | | | | | | | | | | |
| dSpCas9 | | | | | | | | | | | | | |
| with spG4 gRNA | | | | | | | | | | | | | |
| G1333 | C | 0.11% | 0.01% | 99.30% | 99.97% | 99.98% | 99.99% | 0.02% | 0.05% | 0.00% | 99.98% | 99.98% | 99.74% |
| DddAtox-C- | | | | | | | | | | | | | |
| dSpCas9 | | | | | | | | | | | | | |
| with spG4 gRNA | | | | | | | | | | | | | |
| G1333 | G | 99.47% | 99.94% | 0.69% | 0.02% | 0.00% | 0.00% | 0.00% | 0.02% | 0.01% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.05% | 0.01% | 0.33% | 0.01% | 0.00% | 0.24% |
| DddAtox-C- | | | | | | | | | | | | | |
| dSpCas9 | | | | | | | | | | | | | |
| with spG4 gRNA | | | | | | | | | | | | | |
| G1397 | A | 0.20% | 0.04% | 0.01% | 0.00% | 0.00% | 0.01% | 0.01% | 99.94% | 99.65% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | T | 0.23% | 0.03% | 0.01% | 0.00% | 0.01% | 0.01% | 99.93% | 0.01% | 0.00% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.15% | 0.02% | 99.23% | 99.99% | 99.97% | 99.97% | 0.03% | 0.03% | 0.01% | 99.97% | 99.99% | 99.73% |
| DddAtox-N- | | | | | | | | | | | | | |
| dSpCas9 | | | | | | | | | | | | | |
| with spG4 gRNA | | | | | | | | | | | | | |
| G1397 | G | 99.41% | 99.91% | 0.75% | 0.01% | 0.00% | 0.00% | 0.00% | 0.02% | 0.01% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.01% | 0.02% | 0.03% | 0.00% | 0.34% | 0.01% | 0.00% | 0.26% |
| DddAtox-N- | | | | | | | | | | | | | |
| dSpCas9 | | | | | | | | | | | | | |
| with spG4 gRNA | | | | | | | | | | | | | |
| G1397 | A | 0.20% | 0.05% | 0.01% | 0.01% | 0.00% | 0.01% | 0.00% | 99.93% | 99.66% | 0.01% | 0.01% | 0.00% |
| DddAtox-C- | | | | | | | | | | | | | |
| dSpCas9 | | | | | | | | | | | | | |
| with spG4 gRNA | | | | | | | | | | | | | |
| G1397 | T | 0.24% | 0.03% | 0.00% | 0.00% | 0.00% | 0.01% | 99.93% | 0.00% | 0.01% | 0.01% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | C | 0.13% | 0.03% | 99.24% | 99.98% | 99.98% | 99.97% | 0.02% | 0.04% | 0.01% | 99.98% | 99.98% | 99.76% |
| DddAtox-C- | | | | | | | | | | | | | |
| dSpCas9 | | | | | | | | | | | | | |
| with spG4 gRNA | | | | | | | | | | | | | |
| G1397 | G | 99.44% | 99.89% | 0.75% | 0.01% | 0.00% | 0.00% | 0.00% | 0.02% | 0.01% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.01% | 0.01% | 0.04% | 0.01% | 0.31% | 0.01% | 0.01% | 0.23% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | T | A | T | G | T | A | G | C | C | T | C | A |
| G1333 | A | 0.00% | 99.90% | 0.01% | 0.05% | 0.01% | 99.95% | 0.02% | 0.00% | 0.00% | 0.01% | 0.01% | 99.98% |
| DddAtox-N- | | | | | | | | | | | | | |
| dSpCas9 | | | | | | | | | | | | | |
| with spG4 gRNA | | | | | | | | | | | | | |
| G1333 | T | 99.97% | 0.02% | 99.97% | 0.04% | 99.97% | 0.00% | 0.04% | 0.00% | 0.00% | 99.97% | 0.01% | 0.00% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 0.02% | 0.01% | 0.00% | 0.01% | 0.02% | 0.00% | 99.99% | 99.99% | 0.02% | 99.95% | 0.01% |
| DddAtox-N- | | | | | | | | | | | | | |
| dSpCas9 | | | | | | | | | | | | | |
| with spG4 gRNA | | | | | | | | | | | | | |
| G1333 | G | 0.00% | 0.02% | 0.00% | 99.91% | 0.00% | 0.03% | 99.94% | 0.00% | 0.00% | 0.00% | 0.03% | 0.01% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.02% | 0.03% | 0.01% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 0.01% | 99.93% | 0.01% | 0.05% | 0.01% | 99.95% | 0.02% | 0.00% | 0.01% | 0.00% | 0.00% | 99.97% |
| DddAtox-C- | | | | | | | | | | | | | |
| dSpCas9 | | | | | | | | | | | | | |
| with spG4 gRNA | | | | | | | | | | | | | |
| G1333 | T | 99.96% | 0.00% | 99.96% | 0.04% | 99.97% | 0.00% | 0.05% | 0.00% | 0.01% | 99.98% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.01% | 0.02% | 0.02% | 0.00% | 0.01% | 0.01% | 0.00% | 99.99% | 99.98% | 0.01% | 99.96% | 0.01% |
| DddAtox-C- | | | | | | | | | | | | | |
| dSpCas9 | | | | | | | | | | | | | |
| with spG4 gRNA | | | | | | | | | | | | | |
| G1333 | G | 0.00% | 0.01% | 0.00% | 99.90% | 0.00% | 0.03% | 99.93% | 0.00% | 0.00% | 0.00% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.02% | 0.03% | 0.01% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | A | 0.00% | 99.91% | 0.01% | 0.07% | 0.01% | 99.94% | 0.02% | 0.01% | 0.00% | 0.00% | 0.00% | 99.96% |
| DddAtox-N- | | | | | | | | | | | | | |
| dSpCas9 | | | | | | | | | | | | | |
| with spG4 gRNA | | | | | | | | | | | | | |
| G1397 | T | 99.95% | 0.01% | 99.97% | 0.03% | 99.97% | 0.00% | 0.05% | 0.00% | 0.01% | 99.98% | 0.02% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.02% | 0.02% | 0.01% | 0.00% | 0.01% | 0.02% | 0.00% | 99.99% | 99.99% | 0.01% | 99.98% | 0.01% |
| DddAtox-N- | | | | | | | | | | | | | |
| dSpCas9 | | | | | | | | | | | | | |
| with spG4 gRNA | | | | | | | | | | | | | |
| G1397 | G | 0.00% | 0.02% | 0.00% | 99.90% | 0.00% | 0.04% | 99.93% | 0.00% | 0.00% | 0.00% | 0.00% | 0.02% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.02% | 0.04% | 0.01% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 0.00% | 99.88% | 0.00% | 0.04% | 0.02% | 99.94% | 0.02% | 0.01% | 0.00% | 0.01% | 0.01% | 99.96% |
| DddAtox-C- | | | | | | | | | | | | | |
| dSpCas9 | | | | | | | | | | | | | |
| with spG4 gRNA | | | | | | | | | | | | | |
| G1397 | T | 99.96% | 0.02% | 99.98% | 0.03% | 99.96% | 0.00% | 0.05% | 0.00% | 0.01% | 99.97% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.01% | 0.02% | 0.01% | 0.00% | 0.01% | 0.02% | 0.00% | 99.99% | 99.99% | 0.02% | 99.98% | 0.02% |
| DddAtox-C- | | | | | | | | | | | | | |
| dSpCas9 | | | | | | | | | | | | | |
| with spG4 gRNA | | | | | | | | | | | | | |
| G1397 | G | 0.00% | 0.02% | 0.00% | 99.92% | 0.00% | 0.03% | 99.93% | 0.00% | 0.00% | 0.00% | 0.00% | 0.02% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.03% | 0.05% | 0.01% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | G | T | C | T | T | C | C | C | A | T | C | A |
| G1333 | A | 0.01% | 0.01% | 0.01% | 0.00% | 0.00% | 0.02% | 0.01% | 0.01% | 99.95% | 0.01% | 0.01% | 99.96% |
| DddAtox-N- | | | | | | | | | | | | | |
| dSpCas9 | | | | | | | | | | | | | |
| with spG4 gRNA | | | | | | | | | | | | | |
| G1333 | T | 0.04% | 99.98% | 0.01% | 99.96% | 99.95% | 0.01% | 0.01% | 0.01% | 0.01% | 99.96% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 0.01% | 99.98% | 0.04% | 0.01% | 99.97% | 99.95% | 99.97% | 0.01% | 0.01% | 99.98% | 0.01% |
| DddAtox-N- | | | | | | | | | | | | | |
| dSpCas9 | | | | | | | | | | | | | |
| with spG4 gRNA | | | | | | | | | | | | | |
| G1333 | G | 99.94% | 0.00% | 0.00% | 0.00% | 0.03% | 0.00% | 0.03% | 0.00% | 0.03% | 0.03% | 0.00% | 0.01% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 0.00% | 0.01% | 0.00% | 0.00% | 0.00% | 0.01% | 0.00% | 0.01% | 99.95% | 0.01% | 0.00% | 99.96% |
| DddAtox-C- | | | | | | | | | | | | | |
| dSpCas9 | | | | | | | | | | | | | |
| with spG4 gRNA | | | | | | | | | | | | | |
| G1333 | T | 0.04% | 99.98% | 0.01% | 99.97% | 99.96% | 0.01% | 0.01% | 0.01% | 0.01% | 99.97% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.00% | 0.01% | 99.99% | 0.03% | 0.01% | 99.98% | 99.97% | 99.97% | 0.01% | 0.01% | 99.98% | 0.01% |
| DddAtox-C- | | | | | | | | | | | | | |
| dSpCas9 | | | | | | | | | | | | | |
| with spG4 gRNA | | | | | | | | | | | | | |
| G1333 | G | 99.95% | 0.00% | 0.00% | 0.00% | 0.02% | 0.01% | 0.01% | 0.00% | 0.03% | 0.02% | 0.00% | 0.02% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | A | 0.01% | 0.01% | 0.01% | 0.00% | 0.00% | 0.00% | 0.01% | 0.01% | 99.96% | 0.00% | 0.00% | 99.97% |
| DddAtox-N- | | | | | | | | | | | | | |
| dSpCas9 | | | | | | | | | | | | | |
| with spG4 gRNA | | | | | | | | | | | | | |
| G1397 | T | 0.03% | 99.97% | 0.00% | 99.98% | 99.97% | 0.01% | 0.01% | 0.01% | 0.00% | 99.98% | 0.00% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 0.02% | 99.99% | 0.02% | 0.01% | 99.99% | 99.98% | 99.98% | 0.01% | 0.01% | 99.99% | 0.01% |
| DddAtox-N- | | | | | | | | | | | | | |
| dSpCas9 | | | | | | | | | | | | | |
| with spG4 gRNA | | | | | | | | | | | | | |
| G1397 | G | 99.96% | 0.00% | 0.00% | 0.00% | 0.01% | 0.00% | 0.00% | 0.00% | 0.03% | 0.01% | 0.00% | 0.01% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 0.01% | 0.00% | 0.00% | 0.00% | 0.00% | 0.00% | 0.01% | 0.01% | 99.96% | 0.01% | 0.01% | 99.96% |
| DddAtox-C- | | | | | | | | | | | | | |
| dSpCas9 | | | | | | | | | | | | | |
| with spG4 gRNA | | | | | | | | | | | | | |
| G1397 | T | 0.04% | 99.99% | 0.00% | 99.98% | 99.97% | 0.01% | 0.01% | 0.01% | 0.01% | 99.97% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.00% | 0.01% | 99.99% | 0.01% | 0.02% | 99.99% | 99.98% | 99.99% | 0.01% | 0.01% | 99.98% | 0.01% |
| DddAtox-C- | | | | | | | | | | | | | |
| dSpCas9 | | | | | | | | | | | | | |
| with spG4 gRNA | | | | | | | | | | | | | |
| G1397 | G | 99.95% | 0.00% | 0.00% | 0.00% | 0.01% | 0.00% | 0.00% | 0.00% | 0.02% | 0.01% | 0.00% | 0.01% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | G | G | C | T | C | T | C | A | G | C | T | C |
| G1333 | A | 0.01% | 0.04% | 0.00% | 0.02% | 0.00% | 0.01% | 0.01% | 99.95% | 0.01% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | T | 0.04% | 0.01% | 0.01% | 99.97% | 0.01% | 99.97% | 0.01% | 0.00% | 0.01% | 0.01% | 99.97% | 0.01% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 0.00% | 99.96% | 0.01% | 99.98% | 0.01% | 99.96% | 0.01% | 0.00% | 99.98% | 0.01% | 99.97% |
| DddAtox-N- | | | | | | | | | | | | | |
| dSpCas9 | | | | | | | | | | | | | |
| with spG4 gRNA | | | | | | | | | | | | | |
| G1333 | G | 99.95% | 99.93% | 0.03% | 0.00% | 0.00% | 0.00% | 0.02% | 0.04% | 99.98% | 0.00% | 0.02% | 0.00% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 0.01% | 0.02% | 0.00% | 0.00% | 0.01% | 0.01% | 0.01% | 99.96% | 0.02% | 0.00% | 0.01% | 0.00% |
| DddAtox-C- | | | | | | | | | | | | | |
| dSpCas9 | | | | | | | | | | | | | |
| with spG4 gRNA | | | | | | | | | | | | | |
| G1333 | T | 0.04% | 0.00% | 0.00% | 99.97% | 0.00% | 99.98% | 0.00% | 0.00% | 0.01% | 0.00% | 99.96% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.00% | 0.01% | 99.98% | 0.02% | 99.99% | 0.01% | 99.98% | 0.01% | 0.00% | 99.99% | 0.01% | 99.98% |
| DddAtox-C- | | | | | | | | | | | | | |
| dSpCas9 | | | | | | | | | | | | | |
| with spG4 gRNA | | | | | | | | | | | | | |
| G1333 | G | 99.95% | 99.97% | 0.01% | 0.00% | 0.00% | 0.01% | 0.01% | 0.03% | 99.97% | 0.00% | 0.02% | 0.00% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | A | 0.00% | 0.01% | 0.00% | 0.00% | 0.01% | 0.01% | 0.00% | 99.96% | 0.01% | 0.00% | 0.00% | 0.01% |
| DddAtox-N- | | | | | | | | | | | | | |
| dSpCas9 | | | | | | | | | | | | | |
| with spG4 gRNA | | | | | | | | | | | | | |
| G1397 | T | 0.03% | 0.01% | 0.01% | 99.99% | 0.01% | 99.98% | 0.01% | 0.00% | 0.01% | 0.01% | 99.98% | 0.02% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 0.00% | 99.99% | 0.01% | 99.99% | 0.01% | 99.98% | 0.01% | 0.00% | 99.99% | 0.01% | 99.97% |
| DddAtox-N- | | | | | | | | | | | | | |
| dSpCas9 | | | | | | | | | | | | | |
| with spG4 gRNA | | | | | | | | | | | | | |
| G1397 | G | 99.96% | 99.97% | 0.00% | 0.00% | 0.00% | 0.00% | 0.00% | 0.02% | 99.98% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 0.01% | 0.01% | 0.00% | 0.00% | 0.00% | 0.00% | 0.01% | 99.96% | 0.01% | 0.00% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | T | 0.04% | 0.01% | 0.00% | 99.99% | 0.01% | 99.99% | 0.01% | 0.00% | 0.01% | 0.01% | 99.98% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.00% | 0.01% | 100.00% | 0.01% | 99.99% | 0.01% | 99.99% | 0.01% | 0.00% | 99.98% | 0.01% | 99.98% |
| DddAtox-C- | | | | | | | | | | | | | |
| dSpCas9 | | | | | | | | | | | | | |
| with spG4 gRNA | | | | | | | | | | | | | |
| G1397 | G | 99.95% | 99.97% | 0.00% | 0.00% | 0.00% | 0.00% | 0.00% | 0.03% | 99.98% | 0.00% | 0.01% | 0.00% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | A | G | C | C | T | G | A | G | T | G | T | T |
| G1333 | A | 99.95% | 0.00% | 0.03% | 0.01% | 0.00% | 0.01% | 99.94% | 0.01% | 0.02% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | T | 0.00% | 0.00% | 0.01% | 0.00% | 99.97% | 0.01% | 0.01% | 0.01% | 99.92% | 0.03% | 99.92% | 99.95% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 0.01% | 99.96% | 99.98% | 0.01% | 0.01% | 0.01% | 0.01% | 0.04% | 0.01% | 0.02% | 0.02% |
| DddAtox-N- | | | | | | | | | | | | | |
| dSpCas9 | | | | | | | | | | | | | |
| with spG4 gRNA | | | | | | | | | | | | | |
| G1333 | G | 0.04% | 99.98% | 0.00% | 0.00% | 0.00% | 99.97% | 0.04% | 99.98% | 0.01% | 99.94% | 0.04% | 0.01% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.01% | 0.01% | 0.00% | 0.00% | 0.01% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | A | 99.96% | 0.02% | 0.01% | 0.00% | 0.01% | 0.01% | 99.93% | 0.01% | 0.01% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | T | 0.00% | 0.01% | 0.01% | 0.00% | 99.96% | 0.01% | 0.01% | 0.01% | 99.94% | 0.03% | 99.95% | 99.97% |
| DddAtox-C- | | | | | | | | | | | | | |
| dSpCas9 | | | | | | | | | | | | | |
| with spG4 gRNA | | | | | | | | | | | | | |
| G1333 | C | 0.01% | 0.02% | 99.98% | 99.99% | 0.02% | 0.01% | 0.01% | 0.00% | 0.03% | 0.00% | 0.02% | 0.02% |
| DddAtox-C- | | | | | | | | | | | | | |
| dSpCas9 | | | | | | | | | | | | | |
| with spG4 gRNA | | | | | | | | | | | | | |
| G1333 | G | 0.03% | 99.95% | 0.00% | 0.00% | 0.00% | 99.96% | 0.04% | 99.98% | 0.02% | 99.96% | 0.02% | 0.00% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.01% | 0.01% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | A | 99.97% | 0.01% | 0.00% | 0.00% | 0.01% | 0.01% | 99.96% | 0.00% | 0.01% | 0.01% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | T | 0.01% | 0.01% | 0.01% | 0.01% | 99.98% | 0.01% | 0.00% | 0.01% | 99.96% | 0.02% | 99.96% | 99.97% |
| DddAtox-N- | | | | | | | | | | | | | |
| dSpCas9 | | | | | | | | | | | | | |
| with spG4 gRNA | | | | | | | | | | | | | |
| G1397 | C | 0.01% | 0.00% | 99.99% | 99.99% | 0.01% | 0.00% | 0.01% | 0.00% | 0.01% | 0.00% | 0.02% | 0.02% |
| DddAtox-N- | | | | | | | | | | | | | |
| dSpCas9 | | | | | | | | | | | | | |
| with spG4 gRNA | | | | | | | | | | | | | |
| G1397 | G | 0.02% | 99.98% | 0.00% | 0.00% | 0.01% | 99.97% | 0.02% | 99.99% | 0.02% | 99.97% | 0.01% | 0.00% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 99.97% | 0.01% | 0.00% | 0.00% | 0.00% | 0.01% | 99.96% | 0.01% | 0.02% | 0.01% | 0.01% | 0.00% |
| DddAtox-C- | | | | | | | | | | | | | |
| dSpCas9 | | | | | | | | | | | | | |
| with spG4 gRNA | | | | | | | | | | | | | |
| G1397 | T | 0.00% | 0.01% | 0.01% | 0.01% | 99.97% | 0.01% | 0.01% | 0.01% | 99.95% | 0.02% | 99.97% | 99.98% |
| DddAtox-C- | | | | | | | | | | | | | |
| dSpCas9 | | | | | | | | | | | | | |
| with spG4 gRNA | | | | | | | | | | | | | |
| G1397 | C | 0.01% | 0.00% | 99.99% | 99.98% | 0.02% | 0.00% | 0.02% | 0.00% | 0.01% | 0.00% | 0.01% | 0.02% |
| DddAtox-C- | | | | | | | | | | | | | |
| dSpCas9 | | | | | | | | | | | | | |
| with spG4 gRNA | | | | | | | | | | | | | |
| G1397 | G | 0.02% | 99.98% | 0.00% | 0.00% | 0.00% | 99.98% | 0.01% | 99.99% | 0.01% | 99.97% | 0.01% | 0.00% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |

| | Nucle- | | | | | | | | | | | | |
|---|---|---|---|---|---|---|---|---|---|---|---|---|---|
| Batch | otide | G | A | G | G | C | C | C | C | A | G | T | G |
| G1333 | A | 0.04% | 99.93% | 0.02% | 0.01% | 0.00% | 0.02% | 0.01% | 0.01% | 99.94% | 0.02% | 0.02% | 0.02% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.01% | 0.04% | 0.01% | 0.01% | 0.02% | 0.01% | 0.01% | 0.01% | 0.01% | 99.92% | 0.02% |
| DddAtox-N- | | | | | | | | | | | | | |
| dSpCas9 | | | | | | | | | | | | | |
| with spG4 gRNA | | | | | | | | | | | | | |
| G1333 | C | 0.01% | 0.04% | 0.01% | 0.01% | 99.98% | 99.96% | 99.97% | 99.96% | 0.02% | 0.00% | 0.04% | 0.02% |
| DddAtox-N- | | | | | | | | | | | | | |
| dSpCas9 | | | | | | | | | | | | | |
| with spG4 gRNA | | | | | | | | | | | | | |
| G1333 | G | 99.92% | 0.01% | 99.92% | 99.95% | 0.00% | 0.00% | 0.01% | 0.00% | 0.02% | 99.96% | 0.01% | 99.94% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.01% | 0.01% | 0.01% | 0.02% | 0.00% | 0.00% | 0.00% | 0.01% | 0.01% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 0.04% | 99.93% | 0.01% | 0.01% | 0.01% | 0.02% | 0.00% | 0.02% | 99.94% | 0.02% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.00% | 0.05% | 0.01% | 0.02% | 0.01% | 0.01% | 0.02% | 0.01% | 0.02% | 99.93% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.01% | 0.05% | 0.01% | 0.01% | 99.97% | 99.97% | 99.97% | 99.94% | 0.03% | 0.00% | 0.04% | 0.02% |
| DddAtox-C- | | | | | | | | | | | | | |
| dSpCas9 | | | | | | | | | | | | | |
| with spG4 gRNA | | | | | | | | | | | | | |
| G1333 | G | 99.94% | 0.01% | 99.93% | 99.96% | 0.01% | 0.00% | 0.01% | 0.00% | 0.01% | 99.95% | 0.01% | 99.95% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.03% | 0.01% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | A | 0.02% | 99.95% | 0.01% | 0.01% | 0.00% | 0.00% | 0.01% | 0.01% | 99.96% | 0.01% | 0.02% | 0.01% |
| DddAtox-N- | | | | | | | | | | | | | |
| dSpCas9 | | | | | | | | | | | | | |
| with spG4 gRNA | | | | | | | | | | | | | |
| G1397 | T | 0.01% | 0.00% | 0.02% | 0.01% | 0.00% | 0.01% | 0.01% | 0.01% | 0.01% | 0.01% | 99.95% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 0.02% | 0.00% | 0.00% | 99.99% | 99.98% | 99.98% | 99.96% | 0.02% | 0.00% | 0.02% | 0.00% |
| DddAtox-N- | | | | | | | | | | | | | |
| dSpCas9 | | | | | | | | | | | | | |
| with spG4 gRNA | | | | | | | | | | | | | |
| G1397 | G | 99.96% | 0.01% | 99.96% | 99.98% | 0.00% | 0.00% | 0.00% | 0.00% | 0.02% | 99.98% | 0.02% | 99.98% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.01% | 0.00% | 0.00% | 0.00% | 0.00% | 0.00% | 0.00% | 0.02% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 0.01% | 99.95% | 0.01% | 0.01% | 0.00% | 0.00% | 0.01% | 0.01% | 99.95% | 0.01% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.01% | 0.02% | 0.01% | 0.01% | 0.00% | 0.01% | 0.01% | 0.01% | 0.01% | 99.94% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.00% | 0.03% | 0.00% | 0.00% | 99.98% | 100.00 | 99.98% | 99.95% | 0.01% | 0.00% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1397 | G | 99.96% | 0.01% | 99.96% | 99.97% | 0.00% | 0.00% | 0.00% | 0.01% | 0.03% | 99.99% | 0.01% | 99.97% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.01% | 0.00% | 0.00% | 0.00% | 0.00% | 0.00% | 0.00% | 0.02% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |

| | Nucle- | | | | | | | | | | | | |
|---|---|---|---|---|---|---|---|---|---|---|---|---|---|
| Batch | otide | G | C | T | G | C | T | C | T | G | G | G | G |
| G1333 | A | 0.03% | 0.01% | 0.01% | 0.01% | 0.01% | 0.01% | 0.01% | 0.00% | 0.01% | 0.02% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.00% | 99.95% | 0.02% | 0.00% | 99.97% | 0.01% | 99.97% | 0.02% | 0.02% | 0.05% | 0.02% |
| DddAtox-N- | | | | | | | | | | | | | |
| dSpCas9 | | | | | | | | | | | | | |
| with spG4 gRNA | | | | | | | | | | | | | |
| G1333 | C | 0.01% | 99.98% | 0.03% | 0.01% | 99.96% | 0.01% | 99.98% | 0.01% | 0.01% | 0.01% | 0.00% | 0.01% |
| DddAtox-N- | | | | | | | | | | | | | |
| dSpCas9 | | | | | | | | | | | | | |
| with spG4 gRNA | | | | | | | | | | | | | |
| G1333 | G | 99.95% | 0.01% | 0.00% | 99.96% | 0.02% | 0.01% | 0.00% | 0.01% | 99.96% | 99.95% | 99.94% | 99.95% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.01% | 0.00% | 0.00% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | A | 0.01% | 0.01% | 0.02% | 0.01% | 0.00% | 0.00% | 0.01% | 0.00% | 0.01% | 0.01% | 0.01% | 0.02% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.01% | 99.67% | 0.02% | 0.00% | 99.66% | 0.00% | 99.69% | 0.01% | 0.03% | 0.04% | 0.02% |
| DddAtox-C- | | | | | | | | | | | | | |
| dSpCas9 | | | | | | | | | | | | | |
| with spG4 gRNA | | | | | | | | | | | | | |
| G1333 | C | 0.01% | 99.98% | 0.03% | 0.00% | 99.69% | 0.01% | 99.70% | 0.02% | 0.01% | 0.00% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1333 | G | 99.96% | 0.00% | 0.00% | 99.68% | 0.02% | 0.00% | 0.00% | 0.00% | 99.68% | 99.67% | 99.65% | 99.65% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.01% | 0.01% | 0.28% | 0.28% | 0.29% | 0.29% | 0.29% | 0.29% | 0.29% | 0.29% | 0.29% | 0.30% |
| DddAtox-C- | | | | | | | | | | | | | |
| dSpCas9 | | | | | | | | | | | | | |
| with spG4 gRNA | | | | | | | | | | | | | |
| G1397 | A | 0.01% | 0.00% | 0.00% | 0.01% | 0.00% | 0.00% | 0.01% | 0.00% | 0.01% | 0.01% | 0.01% | 0.02% |
| DddAtox-N- | | | | | | | | | | | | | |
| dSpCas9 | | | | | | | | | | | | | |
| with spG4 gRNA | | | | | | | | | | | | | |
| G1397 | T | 0.01% | 0.00% | 99.98% | 0.02% | 0.00% | 99.76% | 0.01% | 99.75% | 0.01% | 0.02% | 0.02% | 0.02% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 99.99% | 0.01% | 0.00% | 99.76% | 0.01% | 99.76% | 0.01% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | G | 99.97% | 0.00% | 0.00% | 99.97% | 0.00% | 0.01% | 0.00% | 0.00% | 99.75% | 99.75% | 99.74% | 99.74% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.22% | 0.22% | 0.23% | 0.23% | 0.22% | 0.22% | 0.22% | 0.23% |
| DddAtox-N- | | | | | | | | | | | | | |
| dSpCas9 | | | | | | | | | | | | | |
| with spG4 gRNA | | | | | | | | | | | | | |
| G1397 | A | 0.00% | 0.00% | 0.01% | 0.01% | 0.01% | 0.00% | 0.01% | 0.01% | 0.01% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.01% | 99.97% | 0.02% | 0.01% | 99.98% | 0.00% | 99.97% | 0.02% | 0.01% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.00% | 99.99% | 0.01% | 0.00% | 99.99% | 0.01% | 99.99% | 0.01% | 0.01% | 0.01% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | G | 99.98% | 0.00% | 0.00% | 99.97% | 0.00% | 0.01% | 0.00% | 0.00% | 99.96% | 99.97% | 99.96% | 99.97% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.01% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | G | C | C | T | C | C | T | G | A | G | T | T |
| G1333 | A | 0.00% | 0.00% | 0.04% | 0.01% | 0.01% | 0.01% | 0.01% | 0.01% | 99.91% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.01% | 0.01% | 99.95% | 0.01% | 0.01% | 99.96% | 0.01% | 0.02% | 0.01% | 99.96% | 99.97% |
| DddAtox-N- | | | | | | | | | | | | | |
| dSpCas9 | | | | | | | | | | | | | |
| with spG4 gRNA | | | | | | | | | | | | | |
| G1333 | C | 0.01% | 99.98% | 99.94% | 0.02% | 99.97% | 99.97% | 0.02% | 0.00% | 0.03% | 0.01% | 0.02% | 0.01% |
| DddAtox-N- | | | | | | | | | | | | | |
| dSpCas9 | | | | | | | | | | | | | |
| with spG4 gRNA | | | | | | | | | | | | | |
| G1333 | G | 99.94% | 0.00% | 0.01% | 0.01% | 0.01% | 0.00% | 0.00% | 99.96% | 0.03% | 99.97% | 0.01% | 0.00% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.04% | 0.00% | 0.01% | 0.01% | 0.01% | 0.01% | 0.01% | 0.01% | 0.01% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | A | 0.01% | 0.00% | 0.08% | 0.01% | 0.01% | 0.01% | 0.01% | 0.02% | 99.62% | 0.00% | 0.01% | 0.00% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.00% | 0.02% | 99.68% | 0.01% | 0.01% | 99.66% | 0.02% | 0.01% | 0.01% | 99.65% | 99.69% |
| DddAtox-C- | | | | | | | | | | | | | |
| dSpCas9 | | | | | | | | | | | | | |
| with spG4 gRNA | | | | | | | | | | | | | |
| G1333 | C | 0.01% | 99.69% | 99.60% | 0.01% | 99.68% | 99.69% | 0.02% | 0.01% | 0.04% | 0.00% | 0.02% | 0.00% |
| DddAtox-C- | | | | | | | | | | | | | |
| dSpCas9 | | | | | | | | | | | | | |
| with spG4 gRNA | | | | | | | | | | | | | |
| G1333 | G | 99.66% | 0.00% | 0.01% | 0.00% | 0.00% | 0.00% | 0.01% | 99.66% | 0.02% | 99.68% | 0.01% | 0.00% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.31% | 0.30% | 0.30% | 0.30% | 0.30% | 0.30% | 0.30% | 0.30% | 0.30% | 0.30% | 0.31% | 0.31% |
| DddAtox-C- | | | | | | | | | | | | | |
| dSpCas9 | | | | | | | | | | | | | |
| with spG4 gRNA | | | | | | | | | | | | | |
| G1397 | A | 0.01% | 0.00% | 0.04% | 0.01% | 0.00% | 0.01% | 0.01% | 0.01% | 99.71% | 0.00% | 0.01% | 0.00% |
| DddAtox-N- | | | | | | | | | | | | | |
| dSpCas9 | | | | | | | | | | | | | |
| with spG4 gRNA | | | | | | | | | | | | | |
| G1397 | T | 0.01% | 0.01% | 0.01% | 99.75% | 0.01% | 0.01% | 99.74% | 0.02% | 0.00% | 0.01% | 99.74% | 99.77% |
| DddAtox-N- | | | | | | | | | | | | | |
| dSpCas9 | | | | | | | | | | | | | |
| with spG4 gRNA | | | | | | | | | | | | | |
| G1397 | C | 0.01% | 99.76% | 99.73% | 0.01% | 99.76% | 99.75% | 0.02% | 0.00% | 0.03% | 0.00% | 0.02% | 0.00% |
| DddAtox-N- | | | | | | | | | | | | | |
| dSpCas9 | | | | | | | | | | | | | |
| with spG4 gRNA | | | | | | | | | | | | | |
| G1397 | G | 99.72% | 0.00% | 0.00% | 0.00% | 0.00% | 0.00% | 0.00% | 99.74% | 0.03% | 99.76% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.26% | 0.23% | 0.23% | 0.23% | 0.23% | 0.23% | 0.23% | 0.23% | 0.23% | 0.23% | 0.23% | 0.23% |
| DddAtox-N- | | | | | | | | | | | | | |
| dSpCas9 | | | | | | | | | | | | | |
| with spG4 gRNA | | | | | | | | | | | | | |
| G1397 | A | 0.01% | 0.00% | 0.02% | 0.01% | 0.00% | 0.01% | 0.01% | 0.01% | 99.91% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.01% | 0.01% | 99.97% | 0.02% | 0.01% | 99.97% | 0.01% | 0.01% | 0.01% | 99.96% | 99.98% |
| DddAtox-C- | | | | | | | | | | | | | |
| dSpCas9 | | | | | | | | | | | | | |
| with spG4 gRNA | | | | | | | | | | | | | |
| G1397 | C | 0.00% | 99.98% | 99.97% | 0.01% | 99.97% | 99.98% | 0.02% | 0.00% | 0.04% | 0.00% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1397 | G | 99.95% | 0.01% | 0.00% | 0.01% | 0.00% | 0.00% | 0.00% | 99.98% | 0.03% | 99.98% | 0.02% | 0.00% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.03% | 0.00% | 0.00% | 0.00% | 0.00% | 0.00% | 0.00% | 0.00% | 0.00% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | T | C | T | C | A | T | C | T | G | T | G | C |
| G1333 | A | 0.00% | 0.00% | 0.01% | 0.01% | 99.95% | 0.01% | 0.01% | 0.02% | 0.02% | 0.02% | 0.04% | 0.01% |
| DddAtox-N- | | | | | | | | | | | | | |
| dSpCas9 | | | | | | | | | | | | | |
| with spG4 gRNA | | | | | | | | | | | | | |
| G1333 | T | 99.97% | 0.02% | 99.95% | 0.01% | 0.01% | 99.97% | 0.01% | 99.96% | 0.02% | 99.92% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | C | 0.01% | 99.96% | 0.01% | 99.97% | 0.02% | 0.01% | 99.96% | 0.01% | 0.00% | 0.03% | 0.01% | 99.97% |
| DddAtox-N- | | | | | | | | | | | | | |
| dSpCas9 | | | | | | | | | | | | | |
| with spG4 gRNA | | | | | | | | | | | | | |
| G1333 | G | 0.00% | 0.01% | 0.02% | 0.00% | 0.02% | 0.00% | 0.01% | 0.00% | 99.96% | 0.02% | 99.93% | 0.00% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.01% | 0.01% | 0.01% | 0.01% | 0.01% | 0.01% | 0.01% | 0.01% | 0.01% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | A | 0.00% | 0.00% | 0.01% | 0.00% | 99.66% | 0.01% | 0.01% | 0.01% | 0.02% | 0.03% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1333 | T | 99.68% | 0.01% | 99.65% | 0.01% | 0.01% | 99.67% | 0.01% | 99.67% | 0.01% | 99.60% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.01% | 99.68% | 0.01% | 99.67% | 0.02% | 0.01% | 99.67% | 0.01% | 0.00% | 0.04% | 0.00% | 99.67% |
| DddAtox-C- | | | | | | | | | | | | | |
| dSpCas9 | | | | | | | | | | | | | |
| with spG4 gRNA | | | | | | | | | | | | | |
| G1333 | G | 0.00% | 0.00% | 0.02% | 0.00% | 0.01% | 0.00% | 0.01% | 0.00% | 99.67% | 0.02% | 99.65% | 0.01% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.31% | 0.31% | 0.31% | 0.31% | 0.31% | 0.31% | 0.31% | 0.31% | 0.30% | 0.30% | 0.30% | 0.31% |
| DddAtox-C- | | | | | | | | | | | | | |
| dSpCas9 | | | | | | | | | | | | | |
| with spG4 gRNA | | | | | | | | | | | | | |
| G1397 | A | 0.00% | 0.00% | 0.01% | 0.01% | 99.75% | 0.00% | 0.01% | 0.01% | 0.01% | 0.02% | 0.02% | 0.00% |
| DddAtox-N- | | | | | | | | | | | | | |
| dSpCas9 | | | | | | | | | | | | | |
| with spG4 gRNA | | | | | | | | | | | | | |
| G1397 | T | 99.76% | 0.00% | 99.74% | 0.01% | 0.00% | 99.76% | 0.01% | 99.76% | 0.01% | 99.71% | 0.02% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.01% | 99.76% | 0.01% | 99.75% | 0.01% | 0.01% | 99.76% | 0.01% | 0.00% | 0.02% | 0.01% | 99.76% |
| DddAtox-N- | | | | | | | | | | | | | |
| dSpCas9 | | | | | | | | | | | | | |
| with spG4 gRNA | | | | | | | | | | | | | |
| G1397 | G | 0.00% | 0.00% | 0.00% | 0.00% | 0.01% | 0.00% | 0.00% | 0.00% | 99.75% | 0.02% | 99.73% | 0.00% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.23% | 0.23% | 0.23% | 0.23% | 0.23% | 0.23% | 0.23% | 0.23% | 0.23% | 0.23% | 0.23% | 0.23% |
| DddAtox-N- | | | | | | | | | | | | | |
| dSpCas9 | | | | | | | | | | | | | |
| with spG4 gRNA | | | | | | | | | | | | | |
| G1397 | A | 0.00% | 0.01% | 0.01% | 0.01% | 99.96% | 0.01% | 0.00% | 0.01% | 0.01% | 0.02% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1397 | T | 99.98% | 0.01% | 99.96% | 0.01% | 0.01% | 99.97% | 0.01% | 99.97% | 0.02% | 99.93% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.01% | 99.97% | 0.01% | 99.97% | 0.02% | 0.01% | 99.98% | 0.01% | 0.00% | 0.02% | 0.01% | 99.97% |
| DddAtox-C- | | | | | | | | | | | | | |
| dSpCas9 | | | | | | | | | | | | | |
| with spG4 gRNA | | | | | | | | | | | | | |
| G1397 | G | 0.00% | 0.01% | 0.01% | 0.00% | 0.01% | 0.00% | 0.00% | 0.00% | 99.96% | 0.01% | 99.95% | 0.00% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.01% | 0.01% | 0.01% | 0.01% | 0.01% | 0.01% | 0.01% | 0.01% | 0.01% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | C | C | C | T | C | C | C | T | C | C | C | T |
| G1333 | A | 0.00% | 0.02% | 0.01% | 0.01% | 0.01% | 0.01% | 0.04% | 0.01% | 0.01% | 0.01% | 0.02% | 0.00% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.01% | 0.02% | 99.95% | 0.02% | 0.02% | 0.01% | 99.97% | 0.02% | 0.01% | 0.00% | 99.92% |
| DddAtox-N- | | | | | | | | | | | | | |
| dSpCas9 | | | | | | | | | | | | | |
| with spG4 gRNA | | | | | | | | | | | | | |
| G1333 | C | 99.97% | 99.96% | 99.95% | 0.02% | 99.97% | 99.96% | 99.93% | 0.01% | 99.95% | 99.95% | 99.94% | 0.03% |
| DddAtox-N- | | | | | | | | | | | | | |
| dSpCas9 | | | | | | | | | | | | | |
| with spG4 gRNA | | | | | | | | | | | | | |
| G1333 | G | 0.00% | 0.00% | 0.00% | 0.01% | 0.00% | 0.00% | 0.00% | 0.00% | 0.00% | 0.00% | 0.00% | 0.01% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.01% | 0.01% | 0.02% | 0.01% | 0.01% | 0.01% | 0.02% | 0.01% | 0.02% | 0.02% | 0.03% | 0.03% |
| DddAtox-N- | | | | | | | | | | | | | |
| dSpCas9 | | | | | | | | | | | | | |
| with spG4 gRNA | | | | | | | | | | | | | |
| G1333 | A | 0.01% | 0.02% | 0.01% | 0.00% | 0.01% | 0.01% | 0.01% | 0.01% | 0.00% | 0.01% | 0.03% | 0.01% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.00% | 0.01% | 99.67% | 0.02% | 0.02% | 0.01% | 99.66% | 0.02% | 0.01% | 0.01% | 99.62% |
| DddAtox-C- | | | | | | | | | | | | | |
| dSpCas9 | | | | | | | | | | | | | |
| with spG4 gRNA | | | | | | | | | | | | | |
| G1333 | C | 99.67% | 99.67% | 99.65% | 0.01% | 99.65% | 99.66% | 99.67% | 0.02% | 99.65% | 99.65% | 99.62% | 0.03% |
| DddAtox-C- | | | | | | | | | | | | | |
| dSpCas9 | | | | | | | | | | | | | |
| with spG4 gRNA | | | | | | | | | | | | | |
| G1333 | G | 0.00% | 0.00% | 0.00% | 0.00% | 0.00% | 0.00% | 0.00% | 0.00% | 0.00% | 0.00% | 0.00% | 0.02% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.31% | 0.31% | 0.32% | 0.31% | 0.31% | 0.31% | 0.31% | 0.31% | 0.33% | 0.33% | 0.33% | 0.33% |
| DddAtox-C- | | | | | | | | | | | | | |
| dSpCas9 | | | | | | | | | | | | | |
| with spG4 gRNA | | | | | | | | | | | | | |
| G1397 | A | 0.00% | 0.01% | 0.01% | 0.01% | 0.01% | 0.01% | 0.03% | 0.00% | 0.01% | 0.03% | 0.02% | 0.01% |
| DddAtox-N- | | | | | | | | | | | | | |
| dSpCas9 | | | | | | | | | | | | | |
| with spG4 gRNA | | | | | | | | | | | | | |
| G1397 | T | 0.00% | 0.01% | 0.00% | 99.73% | 0.02% | 0.02% | 0.01% | 99.75% | 0.02% | 0.01% | 0.00% | 99.71% |
| DddAtox-N- | | | | | | | | | | | | | |
| dSpCas9 | | | | | | | | | | | | | |
| with spG4 gRNA | | | | | | | | | | | | | |
| G1397 | C | 99.76% | 99.76% | 99.74% | 0.02% | 99.74% | 99.74% | 99.73% | 0.01% | 99.73% | 99.72% | 99.73% | 0.03% |
| DddAtox-N- | | | | | | | | | | | | | |
| dSpCas9 | | | | | | | | | | | | | |
| with spG4 gRNA | | | | | | | | | | | | | |
| G1397 | G | 0.00% | 0.00% | 0.00% | 0.00% | 0.00% | 0.00% | 0.00% | 0.00% | 0.00% | 0.00% | 0.00% | 0.01% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.23% | 0.23% | 0.24% | 0.23% | 0.23% | 0.23% | 0.23% | 0.23% | 0.24% | 0.24% | 0.24% | 0.24% |
| DddAtox-N- | | | | | | | | | | | | | |
| dSpCas9 | | | | | | | | | | | | | |
| with spG4 gRNA | | | | | | | | | | | | | |
| G1397 | A | 0.01% | 0.02% | 0.01% | 0.01% | 0.01% | 0.01% | 0.02% | 0.01% | 0.01% | 0.02% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.01% | 0.01% | 99.95% | 0.01% | 0.05% | 0.01% | 99.96% | 0.01% | 0.01% | 0.01% | 99.89% |
| DddAtox-C- | | | | | | | | | | | | | |
| dSpCas9 | | | | | | | | | | | | | |
| with spG4 gRNA | | | | | | | | | | | | | |
| G1397 | C | 99.97% | 99.96% | 99.96% | 0.03% | 99.97% | 99.93% | 99.96% | 0.02% | 99.95% | 99.94% | 99.93% | 0.05% |
| DddAtox-C- | | | | | | | | | | | | | |
| dSpCas9 | | | | | | | | | | | | | |
| with spG4 gRNA | | | | | | | | | | | | | |
| G1397 | G | 0.01% | 0.00% | 0.00% | 0.00% | 0.00% | 0.00% | 0.00% | 0.00% | 0.00% | 0.00% | 0.00% | 0.01% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.01% | 0.01% | 0.02% | 0.01% | 0.01% | 0.01% | 0.01% | 0.01% | 0.03% | 0.03% | 0.04% | 0.04% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | G | G | C | C | C | A | G | G | T | G | A | A |
| G1333 | A | 0.02% | 0.02% | 0.00% | 0.01% | 0.05% | 99.91% | 0.00% | 0.01% | 0.04% | 0.01% | 99.90% | 99.87% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.01% | 0.02% | 0.01% | 0.01% | 0.01% | 0.01% | 0.01% | 99.60% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 0.00% | 99.96% | 99.97% | 99.91% | 0.03% | 0.00% | 0.00% | 0.06% | 0.00% | 0.03% | 0.03% |
| DddAtox-N- | | | | | | | | | | | | | |
| dSpCas9 | | | | | | | | | | | | | |
| with spG4 gRNA | | | | | | | | | | | | | |
| G1333 | G | 99.96% | 99.96% | 0.01% | 0.00% | 0.01% | 0.05% | 99.97% | 99.97% | 0.29% | 99.97% | 0.05% | 0.09% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.01% | 0.01% | 0.01% | 0.01% | 0.01% | 0.01% | 0.01% | 0.01% | 0.01% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | A | 0.01% | 0.01% | 0.01% | 0.00% | 0.02% | 99.86% | 0.00% | 0.01% | 0.02% | 0.01% | 99.90% | 99.84% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.02% | 0.04% | 0.02% | 0.01% | 0.01% | 0.02% | 0.01% | 99.58% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.01% | 0.00% | 99.63% | 99.94% | 99.93% | 0.04% | 0.00% | 0.01% | 0.09% | 0.00% | 0.04% | 0.03% |
| DddAtox-C- | | | | | | | | | | | | | |
| dSpCas9 | | | | | | | | | | | | | |
| with spG4 gRNA | | | | | | | | | | | | | |
| G1333 | G | 99.66% | 99.66% | 0.01% | 0.00% | 0.00% | 0.06% | 99.95% | 99.95% | 0.28% | 99.95% | 0.03% | 0.09% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.31% | 0.31% | 0.31% | 0.03% | 0.03% | 0.03% | 0.02% | 0.02% | 0.02% | 0.02% | 0.02% | 0.02% |
| DddAtox-C- | | | | | | | | | | | | | |
| dSpCas9 | | | | | | | | | | | | | |
| with spG4 gRNA | | | | | | | | | | | | | |
| G1397 | A | 0.01% | 0.01% | 0.00% | 0.00% | 0.01% | 99.67% | 0.00% | 0.01% | 0.04% | 0.01% | 99.69% | 99.64% |
| DddAtox-N- | | | | | | | | | | | | | |
| dSpCas9 | | | | | | | | | | | | | |
| with spG4 gRNA | | | | | | | | | | | | | |
| G1397 | T | 0.02% | 0.01% | 0.02% | 0.01% | 0.02% | 0.01% | 0.01% | 0.01% | 99.38% | 0.02% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 0.00% | 99.74% | 99.76% | 99.74% | 0.04% | 0.00% | 0.00% | 0.07% | 0.00% | 0.04% | 0.03% |
| DddAtox-N- | | | | | | | | | | | | | |
| dSpCas9 | | | | | | | | | | | | | |
| with spG4 gRNA | | | | | | | | | | | | | |
| G1397 | G | 99.74% | 99.75% | 0.01% | 0.00% | 0.00% | 0.05% | 99.76% | 99.76% | 0.29% | 99.75% | 0.04% | 0.09% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.23% | 0.23% | 0.23% | 0.23% | 0.23% | 0.23% | 0.23% | 0.23% | 0.23% | 0.23% | 0.23% | 0.23% |
| DddAtox-N- | | | | | | | | | | | | | |
| dSpCas9 | | | | | | | | | | | | | |
| with spG4 gRNA | | | | | | | | | | | | | |
| G1397 | A | 0.02% | 0.01% | 0.01% | 0.03% | 0.01% | 99.89% | 0.01% | 0.00% | 0.03% | 0.01% | 99.92% | 99.86% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.02% | 0.02% | 0.02% | 0.03% | 0.01% | 0.02% | 0.01% | 99.59% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.01% | 0.00% | 99.96% | 99.94% | 99.94% | 0.03% | 0.01% | 0.00% | 0.08% | 0.01% | 0.04% | 0.04% |
| DddAtox-C- | | | | | | | | | | | | | |
| dSpCas9 | | | | | | | | | | | | | |
| with spG4 gRNA | | | | | | | | | | | | | |
| G1397 | G | 99.96% | 99.96% | 0.01% | 0.01% | 0.00% | 0.06% | 99.96% | 99.97% | 0.29% | 99.96% | 0.03% | 0.09% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.01% | 0.01% | 0.01% | 0.01% | 0.01% | 0.01% | 0.01% | 0.01% | 0.01% | 0.01% | 0.00% | 0.01% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | C | G | T | G | T | G | G | T | T | C | C | A |
| G1333 | A | 0.00% | 0.01% | 0.04% | 0.00% | 0.01% | 0.01% | 0.01% | 0.00% | 0.01% | 0.01% | 0.01% | 99.94% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.01% | 99.57% | 0.02% | 99.89% | 0.02% | 0.01% | 99.94% | 99.96% | 0.01% | 0.02% | 0.01% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 0.00% | 0.06% | 0.00% | 0.03% | 0.00% | 0.00% | 0.03% | 0.01% | 99.97% | 99.95% | 0.02% |
| DddAtox-N- | | | | | | | | | | | | | |
| dSpCas9 | | | | | | | | | | | | | |
| with spG4 gRNA | | | | | | | | | | | | | |
| G1333 | G | 99.98% | 99.97% | 0.33% | 99.97% | 0.06% | 99.96% | 99.97% | 0.02% | 0.00% | 0.00% | 0.01% | 0.03% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.01% | 0.01% | 0.01% | 0.01% | 0.00% | 0.00% | 0.01% | 0.00% | 0.01% | 0.00% | 0.01% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 0.00% | 0.00% | 0.02% | 0.01% | 0.01% | 0.01% | 0.00% | 0.01% | 0.01% | 0.00% | 0.01% | 99.94% |
| DddAtox-C- | | | | | | | | | | | | | |
| dSpCas9 | | | | | | | | | | | | | |
| with spG4 gRNA | | | | | | | | | | | | | |
| G1333 | T | 0.02% | 0.02% | 99.49% | 0.02% | 99.87% | 0.02% | 0.02% | 99.91% | 99.94% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.00% | 0.00% | 0.11% | 0.00% | 0.03% | 0.01% | 0.00% | 0.04% | 0.03% | 99.98% | 99.96% | 0.00% |
| DddAtox-C- | | | | | | | | | | | | | |
| dSpCas9 | | | | | | | | | | | | | |
| with spG4 gRNA | | | | | | | | | | | | | |
| G1333 | G | 99.95% | 99.96% | 0.36% | 99.96% | 0.08% | 99.95% | 99.96% | 0.02% | 0.00% | 0.00% | 0.01% | 0.04% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.02% | 0.02% | 0.02% | 0.02% | 0.02% | 0.01% | 0.01% | 0.01% | 0.02% | 0.01% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1397 | A | 0.01% | 0.00% | 0.04% | 0.01% | 0.02% | 0.01% | 0.00% | 0.01% | 0.00% | 0.00% | 0.01% | 99.95% |
| DddAtox-N- | | | | | | | | | | | | | |
| dSpCas9 | | | | | | | | | | | | | |
| with spG4 gRNA | | | | | | | | | | | | | |
| G1397 | T | 0.01% | 0.01% | 99.37% | 0.02% | 99.65% | 0.02% | 0.01% | 99.94% | 99.97% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 0.00% | 0.07% | 0.00% | 0.03% | 0.00% | 0.00% | 0.03% | 0.02% | 99.98% | 99.98% | 0.01% |
| DddAtox-N- | | | | | | | | | | | | | |
| dSpCas9 | | | | | | | | | | | | | |
| with spG4 gRNA | | | | | | | | | | | | | |
| G1397 | G | 99.75% | 99.76% | 0.29% | 99.75% | 0.07% | 99.75% | 99.99% | 0.02% | 0.00% | 0.00% | 0.00% | 0.03% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.23% | 0.23% | 0.23% | 0.23% | 0.23% | 0.23% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 0.01% | 0.00% | 0.04% | 0.00% | 0.01% | 0.00% | 0.01% | 0.01% | 0.01% | 0.00% | 0.01% | 99.95% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.02% | 99.59% | 0.03% | 99.89% | 0.02% | 0.01% | 99.95% | 99.96% | 0.02% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.01% | 0.00% | 0.07% | 0.01% | 0.03% | 0.00% | 0.01% | 0.02% | 0.02% | 99.97% | 99.97% | 0.01% |
| DddAtox-C- | | | | | | | | | | | | | |
| dSpCas9 | | | | | | | | | | | | | |
| with spG4 gRNA | | | | | | | | | | | | | |
| G1397 | G | 99.97% | 99.98% | 0.30% | 99.96% | 0.07% | 99.98% | 99.98% | 0.02% | 0.00% | 0.01% | 0.00% | 0.03% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.01% | 0.00% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | G | A | A | C | C | G | G | A | G | G | A | C |
| G1333 | A | 0.01% | 99.97% | 99.96% | 0.01% | 0.00% | 0.01% | 0.01% | 99.92% | 0.00% | 0.01% | 99.90% | 0.00% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.00% | 0.00% | 0.01% | 0.01% | 0.01% | 0.01% | 0.01% | 0.01% | 0.02% | 0.02% | 0.01% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 0.01% | 0.02% | 99.98% | 99.97% | 0.00% | 0.00% | 0.01% | 0.00% | 0.00% | 0.06% | 99.99% |
| DddAtox-N- | | | | | | | | | | | | | |
| dSpCas9 | | | | | | | | | | | | | |
| with spG4 gRNA | | | | | | | | | | | | | |
| G1333 | G | 99.97% | 0.01% | 0.01% | 0.00% | 0.01% | 99.97% | 99.98% | 0.06% | 99.98% | 99.97% | 0.03% | 0.00% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 0.01% | 99.97% | 99.96% | 0.01% | 0.00% | 0.01% | 0.01% | 99.88% | 0.00% | 0.01% | 99.88% | 0.01% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.01% | 0.01% | 0.01% | 0.02% | 0.00% | 0.01% | 0.01% | 0.02% | 0.01% | 0.02% | 0.00% |
| DddAtox-C- | | | | | | | | | | | | | |
| dSpCas9 | | | | | | | | | | | | | |
| with spG4 gRNA | | | | | | | | | | | | | |
| G1333 | C | 0.01% | 0.01% | 0.02% | 99.97% | 99.96% | 0.01% | 0.00% | 0.03% | 0.00% | 0.00% | 0.07% | 99.98% |
| DddAtox-C- | | | | | | | | | | | | | |
| dSpCas9 | | | | | | | | | | | | | |
| with spG4 gRNA | | | | | | | | | | | | | |
| G1333 | G | 99.97% | 0.01% | 0.01% | 0.00% | 0.01% | 99.97% | 99.97% | 0.07% | 99.97% | 99.97% | 0.03% | 0.00% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.01% | 0.01% | 0.01% | 0.01% | 0.01% | 0.00% | 0.01% | 0.01% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | A | 0.01% | 99.97% | 99.96% | 0.00% | 0.00% | 0.01% | 0.01% | 99.92% | 0.01% | 0.01% | 99.90% | 0.01% |
| DddAtox-N- | | | | | | | | | | | | | |
| dSpCas9 | | | | | | | | | | | | | |
| with spG4 gRNA | | | | | | | | | | | | | |
| G1397 | T | 0.01% | 0.00% | 0.00% | 0.00% | 0.02% | 0.01% | 0.01% | 0.01% | 0.01% | 0.00% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 0.01% | 0.02% | 99.99% | 99.97% | 0.00% | 0.00% | 0.02% | 0.00% | 0.00% | 0.06% | 99.99% |
| DddAtox-N- | | | | | | | | | | | | | |
| dSpCas9 | | | | | | | | | | | | | |
| with spG4 gRNA | | | | | | | | | | | | | |
| G1397 | G | 99.98% | 0.01% | 0.01% | 0.00% | 0.00% | 99.97% | 99.97% | 0.04% | 99.98% | 99.98% | 0.03% | 0.00% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 0.01% | 99.97% | 99.95% | 0.01% | 0.00% | 0.02% | 0.01% | 99.91% | 0.01% | 0.00% | 99.92% | 0.01% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.00% | 0.01% | 0.01% | 0.01% | 0.02% | 0.01% | 0.01% | 0.01% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.00% | 0.01% | 0.03% | 99.98% | 99.97% | 0.00% | 0.00% | 0.04% | 0.00% | 0.00% | 0.05% | 99.98% |
| DddAtox-C- | | | | | | | | | | | | | |
| dSpCas9 | | | | | | | | | | | | | |
| with spG4 gRNA | | | | | | | | | | | | | |
| G1397 | G | 99.98% | 0.01% | 0.01% | 0.00% | 0.01% | 99.96% | 99.98% | 0.05% | 99.99% | 99.98% | 0.02% | 0.00% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | A | A | A | G | T | A | C | A | A | A | C | G |
| G1333 | A | 99.95% | 99.96% | 99.91% | 0.00% | 0.11% | 99.96% | 0.02% | 99.97% | 99.98% | 99.96% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.01% | 0.00% | 0.01% | 99.77% | 0.01% | 0.01% | 0.00% | 0.01% | 0.00% | 0.01% | 0.02% |
| DddAtox-N- | | | | | | | | | | | | | |
| dSpCas9 | | | | | | | | | | | | | |
| with spG4 gRNA | | | | | | | | | | | | | |
| G1333 | C | 0.02% | 0.02% | 0.04% | 0.00% | 0.05% | 0.01% | 99.97% | 0.02% | 0.01% | 0.02% | 99.98% | 0.00% |
| DddAtox-N- | | | | | | | | | | | | | |
| dSpCas9 | | | | | | | | | | | | | |
| with spG4 gRNA | | | | | | | | | | | | | |
| G1333 | G | 0.02% | 0.01% | 0.04% | 99.99% | 0.06% | 0.02% | 0.00% | 0.01% | 0.01% | 0.01% | 0.01% | 99.97% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 99.95% | 99.95% | 99.90% | 0.01% | 0.13% | 99.96% | 0.03% | 99.97% | 99.97% | 99.96% | 0.01% | 0.02% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.01% | 0.00% | 0.01% | 99.75% | 0.00% | 0.01% | 0.00% | 0.00% | 0.00% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.02% | 0.03% | 0.04% | 0.00% | 0.06% | 0.01% | 99.96% | 0.01% | 0.01% | 0.02% | 99.98% | 0.01% |
| DddAtox-C- | | | | | | | | | | | | | |
| dSpCas9 | | | | | | | | | | | | | |
| with spG4 gRNA | | | | | | | | | | | | | |
| G1333 | G | 0.02% | 0.01% | 0.05% | 99.98% | 0.06% | 0.02% | 0.00% | 0.01% | 0.02% | 0.01% | 0.01% | 99.97% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | A | 99.95% | 99.95% | 99.91% | 0.00% | 0.09% | 99.97% | 0.02% | 99.96% | 99.97% | 99.97% | 0.02% | 0.02% |
| DddAtox-N- | | | | | | | | | | | | | |
| dSpCas9 | | | | | | | | | | | | | |
| with spG4 gRNA | | | | | | | | | | | | | |
| G1397 | T | 0.01% | 0.01% | 0.01% | 0.01% | 99.79% | 0.00% | 0.01% | 0.00% | 0.00% | 0.00% | 0.02% | 0.01% |
| DddAtox-N- | | | | | | | | | | | | | |
| dSpCas9 | | | | | | | | | | | | | |
| with spG4 gRNA | | | | | | | | | | | | | |
| G1397 | C | 0.02% | 0.02% | 0.04% | 0.00% | 0.05% | 0.01% | 99.97% | 0.02% | 0.02% | 0.02% | 99.96% | 0.01% |
| DddAtox-N- | | | | | | | | | | | | | |
| dSpCas9 | | | | | | | | | | | | | |
| with spG4 gRNA | | | | | | | | | | | | | |
| G1397 | G | 0.02% | 0.01% | 0.04% | 99.99% | 0.06% | 0.02% | 0.00% | 0.02% | 0.01% | 0.01% | 0.00% | 99.96% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.01% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 99.94% | 99.95% | 99.90% | 0.01% | 0.11% | 99.96% | 0.02% | 99.97% | 99.97% | 99.95% | 0.01% | 0.02% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.01% | 0.01% | 0.01% | 99.76% | 0.01% | 0.01% | 0.00% | 0.00% | 0.01% | 0.02% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.03% | 0.03% | 0.05% | 0.00% | 0.07% | 0.02% | 99.96% | 0.02% | 0.02% | 0.03% | 99.96% | 0.01% |
| DddAtox-C- | | | | | | | | | | | | | |
| dSpCas9 | | | | | | | | | | | | | |
| with spG4 gRNA | | | | | | | | | | | | | |
| G1397 | G | 0.03% | 0.01% | 0.04% | 99.99% | 0.07% | 0.01% | 0.00% | 0.01% | 0.01% | 0.02% | 0.01% | 99.96% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.01% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | G | C | A | G | A | A | G | C | T | G | G | A |
| G1333 | A | 0.02% | 0.00% | 99.91% | 0.01% | 99.95% | 99.86% | 0.00% | 0.00% | 0.00% | 0.00% | 0.01% | 99.86% |
| DddAtox-N- | | | | | | | | | | | | | |
| dSpCas9 | | | | | | | | | | | | | |
| with spG4 gRNA | | | | | | | | | | | | | |
| G1333 | T | 0.01% | 0.01% | 0.00% | 0.01% | 0.01% | 0.00% | 0.00% | 0.02% | 99.95% | 0.01% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 99.97% | 0.02% | 0.00% | 0.02% | 0.04% | 0.01% | 99.97% | 0.02% | 0.00% | 0.00% | 0.03% |
| DddAtox-N- | | | | | | | | | | | | | |
| dSpCas9 | | | | | | | | | | | | | |
| with spG4 gRNA | | | | | | | | | | | | | |
| G1333 | G | 99.97% | 0.01% | 0.06% | 99.98% | 0.02% | 0.09% | 99.99% | 0.01% | 0.02% | 99.98% | 99.98% | 0.10% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 0.02% | 0.02% | 99.91% | 0.01% | 99.95% | 99.86% | 0.00% | 0.25% | 0.00% | 0.01% | 0.01% | 99.86% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.01% | 0.00% | 0.01% | 0.01% | 0.00% | 0.01% | 0.01% | 99.95% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1333 | C | 0.00% | 99.97% | 0.02% | 0.00% | 0.02% | 0.03% | 0.00% | 99.74% | 0.02% | 0.00% | 0.00% | 0.02% |
| DddAtox-C- | | | | | | | | | | | | | |
| dSpCas9 | | | | | | | | | | | | | |
| with spG4 gRNA | | | | | | | | | | | | | |
| G1333 | G | 99.97% | 0.01% | 0.07% | 99.98% | 0.02% | 0.11% | 99.98% | 0.01% | 0.03% | 99.98% | 99.98% | 0.11% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | A | 0.03% | 0.01% | 99.93% | 0.00% | 99.95% | 99.88% | 0.00% | 0.00% | 0.00% | 0.00% | 0.01% | 99.85% |
| DddAtox-N- | | | | | | | | | | | | | |
| dSpCas9 | | | | | | | | | | | | | |
| with spG4 gRNA | | | | | | | | | | | | | |
| G1397 | T | 0.01% | 0.01% | 0.02% | 0.01% | 0.00% | 0.01% | 0.00% | 0.00% | 99.96% | 0.01% | 0.02% | 0.00% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 99.96% | 0.01% | 0.00% | 0.02% | 0.04% | 0.00% | 99.98% | 0.02% | 0.00% | 0.00% | 0.03% |
| DddAtox-N- | | | | | | | | | | | | | |
| dSpCas9 | | | | | | | | | | | | | |
| with spG4 gRNA | | | | | | | | | | | | | |
| G1397 | G | 99.96% | 0.01% | 0.03% | 99.99% | 0.01% | 0.08% | 99.99% | 0.01% | 0.02% | 99.98% | 99.97% | 0.11% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.01% |
| DddAtox-N- | | | | | | | | | | | | | |
| dSpCas9 | | | | | | | | | | | | | |
| with spG4 gRNA | | | | | | | | | | | | | |
| G1397 | A | 0.02% | 0.01% | 99.92% | 0.00% | 99.95% | 99.87% | 0.00% | 0.01% | 0.01% | 0.00% | 0.01% | 99.84% |
| DddAtox-C- | | | | | | | | | | | | | |
| dSpCas9 | | | | | | | | | | | | | |
| with spG4 gRNA | | | | | | | | | | | | | |
| G1397 | T | 0.01% | 0.01% | 0.01% | 0.01% | 0.00% | 0.00% | 0.01% | 0.01% | 99.95% | 0.01% | 0.01% | 0.01% |
| DddAtox-C- | | | | | | | | | | | | | |
| dSpCas9 | | | | | | | | | | | | | |
| with spG4 gRNA | | | | | | | | | | | | | |
| G1397 | C | 0.00% | 99.96% | 0.02% | 0.00% | 0.03% | 0.03% | 0.00% | 99.98% | 0.02% | 0.00% | 0.01% | 0.05% |
| DddAtox-C- | | | | | | | | | | | | | |
| dSpCas9 | | | | | | | | | | | | | |
| with spG4 gRNA | | | | | | | | | | | | | |
| G1397 | G | 99.96% | 0.02% | 0.04% | 99.99% | 0.02% | 0.09% | 99.98% | 0.01% | 0.02% | 99.98% | 99.97% | 0.10% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| | Nucle- | | | | | | | | | | | | |
| Batch | otide | G | G | A | G | G | A | A | G | G | G | C | C |
| G1333 | A | 0.00% | 0.01% | 99.87% | 0.00% | 0.01% | 99.91% | 99.90% | 0.01% | 0.01% | 0.00% | 0.01% | 0.01% |
| DddAtox-N- | | | | | | | | | | | | | |
| dSpCas9 | | | | | | | | | | | | | |
| with spG4 gRNA | | | | | | | | | | | | | |
| G1333 | T | 0.02% | 0.01% | 0.02% | 0.01% | 0.02% | 0.01% | 0.00% | 0.01% | 0.01% | 0.00% | 0.03% | 0.08% |
| DddAtox-N- | | | | | | | | | | | | | |
| dSpCas9 | | | | | | | | | | | | | |
| with spG4 gRNA | | | | | | | | | | | | | |
| G1333 | C | 0.00% | 0.00% | 0.03% | 0.00% | 0.00% | 0.02% | 0.02% | 0.00% | 0.00% | 0.00% | 99.92% | 99.90% |
| DddAtox-N- | | | | | | | | | | | | | |
| dSpCas9 | | | | | | | | | | | | | |
| with spG4 gRNA | | | | | | | | | | | | | |
| G1333 | G | 99.98% | 99.97% | 0.08% | 99.98% | 99.96% | 0.05% | 0.07% | 99.98% | 99.98% | 99.99% | 0.03% | 0.01% |
| DddAtox-N- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.01% | 0.01% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1333 | A | 0.00% | 0.00% | 99.86% | 0.00% | 0.01% | 99.93% | 99.89% | 0.01% | 0.01% | 0.01% | 0.02% | 0.02% |
| DddAtox-C- | | | | | | | | | | | | | |
| dSpCas9 | | | | | | | | | | | | | |
| with spG4 gRNA | | | | | | | | | | | | | |
| G1333 | T | 0.01% | 0.01% | 0.00% | 0.01% | 0.01% | 0.01% | 0.01% | 0.01% | 0.00% | 0.01% | 0.03% | 0.08% |
| DddAtox-C- | | | | | | | | | | | | | |
| dSpCas9 | | | | | | | | | | | | | |
| with spG4 gRNA | | | | | | | | | | | | | |
| G1333 | C | 0.00% | 0.00% | 0.04% | 0.00% | 0.00% | 0.02% | 0.01% | 0.00% | 0.00% | 0.01% | 99.91% | 99.88% |
| DddAtox-C- | | | | | | | | | | | | | |
| dSpCas9 | | | | | | | | | | | | | |
| with spG4 gRNA | | | | | | | | | | | | | |
| G1333 | G | 99.99% | 99.98% | 0.10% | 99.98% | 99.98% | 0.05% | 0.09% | 99.98% | 99.98% | 99.97% | 0.04% | 0.01% |
| DddAtox-C- | | | | | | | | | | | | | |
| dSpCas9 | | | | | | | | | | | | | |
| with spG4 gRNA | | | | | | | | | | | | | |
| G1333 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1333 | — | 0.00% | 0.00% | 0.00% | 0.00% | 0.00% | 0.00% | 0.00% | 0.00% | 0.00% | 0.00% | 0.00% | 0.01% |
| DddAtox-C- | | | | | | | | | | | | | |
| dSpCas9 | | | | | | | | | | | | | |
| with spG4 gRNA | | | | | | | | | | | | | |
| G1397 | A | 0.00% | 0.00% | 99.88% | 0.01% | 0.01% | 99.91% | 99.91% | 0.01% | 0.01% | 0.00% | 0.02% | 0.03% |
| DddAtox-N- | | | | | | | | | | | | | |
| dSpCas9 | | | | | | | | | | | | | |
| with spG4 gRNA | | | | | | | | | | | | | |
| G1397 | T | 0.02% | 0.01% | 0.02% | 0.01% | 0.01% | 0.01% | 0.01% | 0.01% | 0.01% | 0.01% | 0.04% | 0.06% |
| DddAtox-N- | | | | | | | | | | | | | |
| dSpCas9 | | | | | | | | | | | | | |
| with spG4 gRNA | | | | | | | | | | | | | |
| G1397 | C | 0.00% | 0.00% | 0.04% | 0.00% | 0.00% | 0.03% | 0.03% | 0.00% | 0.00% | 0.00% | 99.90% | 99.89% |
| DddAtox-N- | | | | | | | | | | | | | |
| dSpCas9 | | | | | | | | | | | | | |
| with spG4 gRNA | | | | | | | | | | | | | |
| G1397 | G | 99.97% | 99.98% | 0.06% | 99.97% | 99.98% | 0.05% | 0.05% | 99.97% | 99.98% | 99.98% | 0.03% | 0.02% |
| DddAtox-N- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.01% | 0.01% | 0.01% | 0.00% | 0.00% | 0.00% | 0.00% | 0.00% | 0.00% |
| DddAtox-N- | | | | | | | | | | | | | |
| dSpCas9 | | | | | | | | | | | | | |
| with spG4 gRNA | | | | | | | | | | | | | |
| G1397 | A | 0.01% | 0.00% | 99.84% | 0.01% | 0.01% | 99.94% | 99.91% | 0.01% | 0.01% | 0.01% | 0.02% | 0.02% |
| DddAtox-C- | | | | | | | | | | | | | |
| dSpCas9 | | | | | | | | | | | | | |
| with spG4 gRNA | | | | | | | | | | | | | |
| G1397 | T | 0.02% | 0.01% | 0.01% | 0.01% | 0.01% | 0.01% | 0.01% | 0.01% | 0.01% | 0.01% | 0.02% | 0.07% |
| DddAtox-C- | | | | | | | | | | | | | |
| dSpCas9 | | | | | | | | | | | | | |
| with spG4 gRNA | | | | | | | | | | | | | |
| G1397 | C | 0.00% | 0.00% | 0.05% | 0.00% | 0.00% | 0.01% | 0.02% | 0.00% | 0.00% | 0.00% | 99.92% | 99.88% |
| DddAtox-C- | | | | | | | | | | | | | |
| dSpCas9 | | | | | | | | | | | | | |
| with spG4 gRNA | | | | | | | | | | | | | |
| G1397 | G | 99.97% | 99.98% | 0.09% | 99.97% | 99.97% | 0.03% | 0.07% | 99.97% | 99.98% | 99.98% | 0.04% | 0.02% |
| DddAtox-C- | | | | | | | | | | | | | |
| dSpCas9 | | | | | | | | | | | | | |
| with spG4 gRNA | | | | | | | | | | | | | |
| G1397 | N | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |
| G1397 | — | 0.00% | 0.00% | 0.00% | 0.00% | 0.00% | 0.00% | 0.00% | 0.00% | 0.00% | 0.00% | 0.00% | 0.00% |
| DddAtox-C- | | | | | | | | | | | | | |
| dSpCas9 | | | | | | | | | | | | | |
| with spG4 gRNA | | | | | | | | | | | | | |

| Batch | Nucleotide | T | G | A | G | T | C | C | G |
|---|---|---|---|---|---|---|---|---|---|
| G1333 DddAtox-N- | A | 0.00% | 0.01% | 99.87% | 0.01% | 0.01% | 0.01% | 0.01% | 0.01% |
| dSpCas9 with spG4 gRNA | | | | | | | | | |
| G1333 DddAtox-N- | T | 99.91% | 0.01% | 0.00% | 0.00% | 99.84% | 0.03% | 0.04% | 0.01% |
| dSpCas9 with spG4 gRNA | | | | | | | | | |
| G1333 DddAtox-N- | C | 0.05% | 0.00% | 0.03% | 0.00% | 0.05% | 99.96% | 99.86% | 0.00% |
| dSpCas9 with spG4 gRNA | | | | | | | | | |
| G1333 DddAtox-N- | G | 0.04% | 99.97% | 0.08% | 99.97% | 0.05% | 0.00% | 0.01% | 99.93% |
| dSpCas9 with spG4 gRNA | | | | | | | | | |
| G1333 DddAtox-N- | N | 0.00% | 0.00% | 0.00% | 0.00% | 0.00% | 0.00% | 0.00% | 0.00% |
| dSpCas9 with spG4 gRNA | | | | | | | | | |
| G1333 DddAtox-N- | — | 0.00% | 0.00% | 0.00% | 0.01% | 0.05% | 0.00% | 0.08% | 0.05% |
| dSpCas9 with spG4 gRNA | | | | | | | | | |
| G1333 DddAtox-C- | A | 0.01% | 0.03% | 99.89% | 0.01% | 0.01% | 0.03% | 0.01% | 0.02% |
| dSpCas9 with spG4 gRNA | | | | | | | | | |
| G1333 DddAtox-C- | T | 99.90% | 0.01% | 0.01% | 0.02% | 99.81% | 0.03% | 0.05% | 0.01% |
| dSpCas9 with spG4 gRNA | | | | | | | | | |
| G1333 DddAtox-C- | C | 0.04% | 0.00% | 0.02% | 0.00% | 0.09% | 99.93% | 99.82% | 0.01% |
| dSpCas9 with spG4 gRNA | | | | | | | | | |
| G1333 DddAtox-C- | G | 0.04% | 99.96% | 0.07% | 99.96% | 0.06% | 0.01% | 0.02% | 99.90% |
| dSpCas9 with spG4 gRNA | | | | | | | | | |
| G1333 DddAtox-C- | N | 0.00% | 0.00% | 0.00% | 0.00% | 0.00% | 0.00% | 0.00% | 0.00% |
| dSpCas9 with spG4 gRNA | | | | | | | | | |
| G1333 DddAtox-C- | — | 0.00% | 0.00% | 0.00% | 0.02% | 0.03% | 0.00% | 0.10% | 0.07% |
| dSpCas9 with spG4 gRNA | | | | | | | | | |
| G1397 DddAtox-N- | A | 0.01% | 0.01% | 99.86% | 0.00% | 0.02% | 0.01% | 0.01% | 0.01% |
| dSpCas9 with spG4 gRNA | | | | | | | | | |
| G1397 DddAtox-N- | T | 99.91% | 0.02% | 0.01% | 0.01% | 99.81% | 0.05% | 0.05% | 0.01% |
| dSpCas9 with spG4 gRNA | | | | | | | | | |
| G1397 DddAtox-N- | C | 0.04% | 0.00% | 0.03% | 0.00% | 0.07% | 99.93% | 99.86% | 0.00% |
| dSpCas9 with spG4 gRNA | | | | | | | | | |
| G1397 DddAtox-N- | G | 0.04% | 99.96% | 0.09% | 99.95% | 0.05% | 0.01% | 0.01% | 99.89% |
| dSpCas9 with spG4 gRNA | | | | | | | | | |
| G1397 DddAtox-N- | N | 0.00% | 0.00% | 0.00% | 0.00% | 0.00% | 0.00% | 0.00% | 0.00% |
| dSpCas9 with spG4 gRNA | | | | | | | | | |
| G1397 DddAtox-N- | — | 0.00% | 0.00% | 0.01% | 0.03% | 0.05% | 0.00% | 0.08% | 0.08% |
| dSpCas9 with spG4 gRNA | | | | | | | | | |
| G1397 DddAtox-C- | A | 0.01% | 0.01% | 99.86% | 0.01% | 0.03% | 0.01% | 0.00% | 0.01% |
| dSpCas9 with spG4 gRNA | | | | | | | | | |
| G1397 DddAtox-C- | T | 99.89% | 0.02% | 0.01% | 0.01% | 99.82% | 0.04% | 0.05% | 0.01% |
| dSpCas9 with spG4 gRNA | | | | | | | | | |
| G1397 DddAtox-C- | C | 0.05% | 0.00% | 0.04% | 0.00% | 0.07% | 99.93% | 99.85% | 0.00% |
| dSpCas9 with spG4 gRNA | | | | | | | | | |
| G1397 DddAtox-C- | G | 0.04% | 99.97% | 0.09% | 99.95% | 0.05% | 0.01% | 0.01% | 99.89% |
| dSpCas9 with spG4 gRNA | | | | | | | | | |
| G1397 DddAtox-C- | N | 0.00% | 0.00% | 0.00% | 0.00% | 0.00% | 0.00% | 0.00% | 0.00% |
| dSpCas9 with spG4 gRNA | | | | | | | | | |
| G1397 DddAtox-C- | — | 0.01% | 0.00% | 0.01% | 0.03% | 0.05% | 0.00% | 0.09% | 0.08% |
| dSpCas9 with spG4 gRNA | | | | | | | | | |

| Batch | Nucleotide | A | G | C | A | G | A | A | G |
|---|---|---|---|---|---|---|---|---|---|
| G1333 DddAtox-N- | A | 99.88% | 0.01% | 0.03% | 1.92% | 0.07% | 1.81% | 0.03% | 0.00% |
| dSpCas9 with spG4 gRNA | | | | | | | | | |
| G1333 DddAtox-N- | T | 0.01% | 0.01% | 0.03% | 0.01% | 0.01% | 0.01% | 0.00% | 0.00% |
| dSpCas9 with spG4 gRNA | | | | | | | | | |
| G1333 DddAtox-N- | C | 0.02% | 0.00% | 99.90% | 0.04% | 0.00% | 0.01% | 0.00% | 0.00% |
| dSpCas9 with spG4 gRNA | | | | | | | | | |
| G1333 DddAtox-N- | G | 0.07% | 99.94% | 0.02% | 0.09% | 1.97% | 0.04% | 0.00% | 0.03% |
| dSpCas9 with spG4 gRNA | | | | | | | | | |
| G1333 DddAtox-N- | N | 0.00% | 0.00% | 0.00% | 0.00% | 0.00% | 0.00% | 0.00% | 0.00% |
| dSpCas9 with spG4 gRNA | | | | | | | | | |
| G1333 DddAtox-N- | — | 0.02% | 0.04% | 0.03% | 97.94% | 97.96% | 98.13% | 99.97% | 99.97% |
| dSpCas9 with spG4 gRNA | | | | | | | | | |
| G1333 DddAtox-C- | A | 99.86% | 0.01% | 0.02% | 2.35 | 0.09% | 2.23 | 0.34 | 0.00% |
| dSpCas9 with spG4 gRNA | | | | | | | | | |
| G1333 DddAtox-C- | T | 0.01% | 0.00% | 0.05% | 0.01% | 0.00% | 0.01% | 0.00% | 0.00% |
| dSpCas9 with spG4 gRNA | | | | | | | | | |
| G1333 DddAtox-C- | C | 0.02% | 0.00% | 99.89% | 0.06% | 0.00% | 0.00% | 0.00% | 0.00% |
| dSpCas9 with spG4 gRNA | | | | | | | | | |
| G1333 DddAtox-C- | G | 0.08% | 99.96% | 0.02% | 0.11% | 2.39% | 0.04% | 0.00% | 0.34% |
| dSpCas9 with spG4 gRNA | | | | | | | | | |
| G1333 DddAtox-C- | N | 0.00% | 0.00% | 0.00% | 0.00% | 0.00% | 0.00% | 0.00% | 0.00% |
| dSpCas9 with spG4 gRNA | | | | | | | | | |
| G1333 DddAtox-C- | — | 0.03% | 0.03% | 0.02% | 97.48% | 97.52% | 97.72% | 99.66% | 99.66% |
| dSpCas9 with spG4 gRNA | | | | | | | | | |
| G1397 DddAtox-N- | A | 99.87% | 0.01% | 0.02% | 2.42% | 0.10% | 2.29% | 0.25% | 0.00% |
| dSpCas9 with spG4 gRNA | | | | | | | | | |
| G1397 DddAtox-N- | T | 0.01% | 0.00% | 0.04% | 0.01% | 0.00% | 0.01% | 0.00% | 0.00% |
| dSpCas9 with spG4 gRNA | | | | | | | | | |
| G1397 DddAtox-N- | C | 0.02% | 0.01% | 99.90% | 0.06% | 0.00% | 0.01% | 0.00% | 0.00% |
| dSpCas9 with spG4 gRNA | | | | | | | | | |
| G1397 DddAtox-N- | G | 0.08% | 99.94% | 0.02% | 0.09% | 2.46% | 0.06% | 0.00% | 0.25% |
| dSpCas9 with spG4 gRNA | | | | | | | | | |
| G1397 DddAtox-N- | N | 0.00% | 0.00% | 0.00% | 0.00% | 0.00% | 0.00% | 0.00% | 0.00% |
| dSpCas9 with spG4 gRNA | | | | | | | | | |
| G1397 DddAtox-N- | — | 0.03% | 0.04% | 0.02% | 97.42% | 97.44% | 97.64% | 99.75% | 99.75% |
| dSpCas9 with spG4 gRNA | | | | | | | | | |
| G1397 DddAtox-C- | A | 99.86% | 0.01% | 0.03% | 2.19% | 0.10% | 2.05% | 0.04% | 0.00% |
| dSpCas9 with spG4 gRNA | | | | | | | | | |
| G1397 DddAtox-C- | T | 0.01% | 0.01% | 0.04% | 0.01% | 0.01% | 0.00% | 0.00% | 0.00% |
| dSpCas9 with spG4 gRNA | | | | | | | | | |
| G1397 DddAtox-C- | C | 0.03% | 0.01% | 99.88% | 0.04% | 0.00% | 0.01% | 0.00% | 0.00% |
| dSpCas9 with spG4 gRNA | | | | | | | | | |
| G1397 DddAtox-C- | G | 0.06% | 99.93% | 0.03% | 0.10% | 2.20% | 0.04% | 0.00% | 0.04% |
| dSpCas9 with spG4 gRNA | | | | | | | | | |
| G1397 DddAtox-C- | N | 0.00% | 0.00% | 0.00% | 0.00% | 0.00% | 0.00% | 0.00% | 0.00% |
| dSpCas9 with spG4 gRNA | | | | | | | | | |
| G1397 DddAtox-C- | — | 0.03% | 0.04% | 0.02% | 97.66% | 97.69% | 97.90% | 99.95% | 99.96% |
| dSpCas9 with spG4 gRNA | | | | | | | | | |

TABLE 10C
No gRNA

| | Nucle- | | | | | | | | | | | | |
|---|---|---|---|---|---|---|---|---|---|---|---|---|---|
| Batch | otide | G | G | C | C | C | C | T | A | A | C | C | C |
| nogRNA- | A | 0.23% | 0.03% | 0.01% | 0.00% | 0.00% | 0.02% | 0.00% | 99.89% | 99.60% | 0.00% | 0.00% | 0.01% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | T | 0.25% | 0.04% | 0.01% | 0.01% | 0.01% | 0.00% | 99.89% | 0.02% | 0.01% | 0.00% | 0.00% | 0.00% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | C | 0.18% | 0.01% | 99.17% | 99.97% | 99.98% | 99.97% | 0.06% | 0.03% | 0.00% | 100.00% | 99.98% | 99.72% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | G | 99.34% | 99.92% | 0.80% | 0.01% | 0.00% | 0.00% | 0.00% | 0.05% | 0.04% | 0.00% | 0.00% | 0.00% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | — | 0.00% | 0.00% | 0.00% | 0.00% | 0.00% | 0.00% | 0.05% | 0.00% | 0.34% | 0.00% | 0.01% | 0.27% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | A | 0.25% | 0.04% | 0.01% | 0.00% | 0.00% | 0.00% | 0.00% | 99.92% | 99.56% | 0.01% | 0.00% | 0.01% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | T | 0.27% | 0.03% | 0.00% | 0.01% | 0.01% | 0.00% | 99.93% | 0.00% | 0.00% | 0.01% | 0.02% | 0.00% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | C | 0.15% | 0.03% | 99.10% | 99.98% | 99.98% | 99.99% | 0.01% | 0.05% | 0.03% | 99.96% | 99.97% | 99.69% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | G | 99.33% | 99.90% | 0.89% | 0.00% | 0.00% | 0.00% | 0.00% | 0.04% | 0.02% | 0.00% | 0.00% | 0.00% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | — | 0.00% | 0.00% | 0.00% | 0.00% | 0.00% | 0.00% | 0.05% | 0.00% | 0.39% | 0.01% | 0.00% | 0.29% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | A | 0.21% | 0.05% | 0.02% | 0.00% | 0.01% | 0.01% | 0.00% | 99.94% | 99.59% | 0.00% | 0.00% | 0.01% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | T | 0.25% | 0.01% | 0.01% | 0.01% | 0.01% | 0.00% | 99.90% | 0.01% | 0.01% | 0.01% | 0.01% | 0.01% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | C | 0.14% | 0.02% | 99.18% | 99.98% | 99.96% | 99.97% | 0.04% | 0.02% | 0.02% | 99.98% | 99.97% | 99.74% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | G | 99.40% | 99.91% | 0.79% | 0.01% | 0.00% | 0.00% | 0.00% | 0.02% | 0.02% | 0.00% | 0.00% | 0.00% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | — | 0.00% | 0.00% | 0.00% | 0.00% | 0.01% | 0.02% | 0.05% | 0.01% | 0.37% | 0.01% | 0.01% | 0.24% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | A | 0.24% | 0.03% | 0.00% | 0.00% | 0.00% | 0.02% | 0.00% | 99.90% | 99.57% | 0.01% | 0.00% | 0.00% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | T | 0.27% | 0.03% | 0.01% | 0.00% | 0.00% | 0.02% | 99.93% | 0.00% | 0.01% | 0.00% | 0.00% | 0.00% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | C | 0.14% | 0.01% | 99.11% | 99.97% | 99.99% | 99.96% | 0.02% | 0.03% | 0.01% | 99.98% | 99.99% | 99.73% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | G | 99.35% | 99.92% | 0.87% | 0.03% | 0.00% | 0.01% | 0.00% | 0.06% | 0.03% | 0.00% | 0.00% | 0.00% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | — | 0.00% | 0.00% | 0.00% | 0.00% | 0.00% | 0.00% | 0.04% | 0.01% | 0.38% | 0.00% | 0.01% | 0.27% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | A | 0.27% | 0.03% | 0.01% | 0.00% | 0.01% | 0.00% | 0.00% | 99.89% | 99.56% | 0.01% | 0.01% | 0.00% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | T | 0.28% | 0.01% | 0.00% | 0.01% | 0.01% | 0.01% | 99.93% | 0.00% | 0.00% | 0.00% | 0.01% | 0.00% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | C | 0.19% | 0.02% | 99.00% | 99.97% | 99.97% | 99.96% | 0.02% | 0.03% | 0.01% | 99.97% | 99.96% | 99.65% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | G | 99.25% | 99.93% | 0.98% | 0.01% | 0.00% | 0.01% | 0.01% | 0.07% | 0.03% | 0.00% | 0.00% | 0.00% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | — | 0.01% | 0.00% | 0.00% | 0.00% | 0.01% | 0.02% | 0.05% | 0.00% | 0.40% | 0.01% | 0.02% | 0.34% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | A | 0.20% | 0.03% | 0.02% | 0.00% | 0.00% | 0.00% | 0.01% | 99.93% | 99.52% | 0.00% | 0.01% | 0.00% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | T | 0.35% | 0.02% | 0.00% | 0.01% | 0.00% | 0.01% | 99.90% | 0.01% | 0.00% | 0.00% | 0.02% | 0.01% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | C | 0.19% | 0.03% | 99.03% | 99.96% | 99.95% | 99.95% | 0.05% | 0.03% | 0.03% | 99.98% | 99.95% | 99.72% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | G | 99.26% | 99.92% | 0.95% | 0.02% | 0.01% | 0.00% | 0.00% | 0.02% | 0.01% | 0.00% | 0.00% | 0.00% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | — | 0.00% | 0.00% | 0.00% | 0.00% | 0.04% | 0.04% | 0.04% | 0.01% | 0.44% | 0.02% | 0.02% | 0.26% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | A | 0.18% | 0.05% | 0.02% | 0.01% | 0.01% | 0.01% | 0.00% | 99.93% | 99.62% | 0.00% | 0.00% | 0.00% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | T | 0.24% | 0.03% | 0.01% | 0.02% | 0.00% | 0.00% | 99.95% | 0.01% | 0.00% | 0.01% | 0.02% | 0.02% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | C | 0.16% | 0.02% | 99.25% | 99.96% | 99.98% | 99.97% | 0.02% | 0.01% | 0.01% | 99.97% | 99.98% | 99.66% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | G | 99.42% | 99.90% | 0.71% | 0.01% | 0.00% | 0.00% | 0.00% | 0.04% | 0.01% | 0.00% | 0.00% | 0.00% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | — | 0.00% | 0.00% | 0.00% | 0.00% | 0.01% | 0.01% | 0.03% | 0.01% | 0.36% | 0.02% | 0.00% | 0.33% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | A | 0.20% | 0.06% | 0.00% | 0.00% | 0.01% | 0.00% | 0.00% | 99.92% | 99.61% | 0.01% | 0.00% | 0.00% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | T | 0.28% | 0.03% | 0.02% | 0.02% | 0.01% | 0.00% | 99.93% | 0.00% | 0.01% | 0.01% | 0.00% | 0.01% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | C | 0.16% | 0.02% | 99.09% | 99.96% | 99.97% | 99.98% | 0.02% | 0.02% | 0.01% | 99.96% | 99.98% | 99.70% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | G | 99.35% | 99.89% | 0.89% | 0.01% | 0.00% | 0.00% | 0.02% | 0.05% | 0.02% | 0.00% | 0.01% | 0.00% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | — | 0.00% | 0.00% | 0.00% | 0.00% | 0.01% | 0.01% | 0.03% | 0.00% | 0.35% | 0.01% | 0.01% | 0.29% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | A | 0.22% | 0.06% | 0.01% | 0.01% | 0.00% | 0.00% | 0.00% | 99.88% | 99.58% | 0.01% | 0.00% | 0.00% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | T | 0.26% | 0.01% | 0.01% | 0.00% | 0.00% | 0.00% | 99.92% | 0.01% | 0.01% | 0.00% | 0.00% | 0.00% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | C | 0.19% | 0.03% | 99.10% | 99.97% | 99.98% | 99.98% | 0.03% | 0.05% | 0.01% | 99.98% | 99.98% | 99.74% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | G | 99.31% | 99.90% | 0.87% | 0.02% | 0.00% | 0.00% | 0.00% | 0.06% | 0.02% | 0.00% | 0.00% | 0.00% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | — | 0.01% | 0.00% | 0.00% | 0.00% | 0.01% | 0.01% | 0.05% | 0.01% | 0.38% | 0.01% | 0.01% | 0.25% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | A | 0.27% | 0.04% | 0.01% | 0.00% | 0.01% | 0.01% | 0.01% | 99.92% | 99.56% | 0.00% | 0.00% | 0.01% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | T | 0.29% | 0.02% | 0.01% | 0.01% | 0.01% | 0.00% | 99.92% | 0.01% | 0.01% | 0.01% | 0.02% | 0.00% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | C | 0.15% | 0.03% | 98.97% | 99.96% | 99.98% | 99.98% | 0.02% | 0.02% | 0.01% | 99.96% | 99.97% | 99.76% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | G | 99.29% | 99.90% | 1.01% | 0.03% | 0.00% | 0.00% | 0.00% | 0.03% | 0.03% | 0.00% | 0.00% | 0.00% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | — | 0.00% | 0.00% | 0.00% | 0.00% | 0.01% | 0.01% | 0.05% | 0.02% | 0.39% | 0.02% | 0.01% | 0.23% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | A | 0.26% | 0.04% | 0.01% | 0.00% | 0.00% | 0.01% | 0.00% | 99.93% | 99.59% | 0.01% | 0.00% | 0.01% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | T | 0.32% | 0.02% | 0.01% | 0.01% | 0.02% | 0.01% | 99.94% | 0.00% | 0.00% | 0.01% | 0.01% | 0.00% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | C | 0.17% | 0.01% | 99.09% | 99.97% | 99.97% | 99.97% | 0.01% | 0.03% | 0.00% | 99.97% | 99.99% | 99.73% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | G | 99.25% | 99.92% | 0.89% | 0.01% | 0.00% | 0.00% | 0.00% | 0.02% | 0.02% | 0.00% | 0.00% | 0.00% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | — | 0.00% | 0.00% | 0.00% | 0.00% | 0.01% | 0.01% | 0.05% | 0.01% | 0.38% | 0.01% | 0.00% | 0.27% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | A | 0.22% | 0.03% | 0.03% | 0.01% | 0.00% | 0.00% | 0.01% | 99.85% | 99.57% | 0.00% | 0.01% | 0.00% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | T | 0.23% | 0.02% | 0.01% | 0.01% | 0.00% | 0.00% | 99.94% | 0.01% | 0.01% | 0.00% | 0.01% | 0.01% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | C | 0.19% | 0.03% | 99.18% | 99.96% | 99.97% | 99.98% | 0.02% | 0.05% | 0.03% | 99.99% | 99.98% | 99.76% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | G | 99.37% | 99.93% | 0.78% | 0.02% | 0.01% | 0.00% | 0.00% | 0.08 | 0.03% | 0.00% | 0.00% | 0.01% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | — | 0.00% | 0.00% | 0.00% | 0.00% | 0.02% | 0.02% | 0.03% | 0.02% | 0.36% | 0.01% | 0.01% | 0.23% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | A | 0.30% | 0.04% | 0.02% | 0.00% | 0.01% | 0.01% | 0.00% | 99.90% | 99.52% | 0.00% | 0.00% | 0.00% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | T | 0.28% | 0.03% | 0.01% | 0.01% | 0.01% | 0.00% | 99.93% | 0.00% | 0.00% | 0.01% | 0.00% | 0.02% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | C | 0.13% | 0.02% | 99.06% | 99.96% | 99.96% | 99.96% | 0.02% | 0.04% | 0.01% | 99.98% | 99.98% | 99.64% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | G | 99.28% | 99.91% | 0.91% | 0.02% | 0.00% | 0.00% | 0.01% | 0.04% | 0.03% | 0.00% | 0.00% | 0.00% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | — | 0.00% | 0.00% | 0.00% | 0.00% | 0.01% | 0.03% | 0.04% | 0.01% | 0.44% | 0.01% | 0.01% | 0.33% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | A | 0.26% | 0.04% | 0.01% | 0.01% | 0.01% | 0.01% | 0.00% | 99.90% | 99.56% | 0.01% | 0.00% | 0.00% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | T | 0.28% | 0.02% | 0.01% | 0.02% | 0.01% | 0.02% | 99.94% | 0.00% | 0.01% | 0.00% | 0.01% | 0.00% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | C | 0.20% | 0.03% | 99.09% | 99.96% | 99.96% | 99.94% | 0.02% | 0.04% | 0.01% | 99.99% | 99.99% | 99.80% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | G | 99.25% | 99.90% | 0.89% | 0.02% | 0.00% | 0.00% | 0.00% | 0.05% | 0.01% | 0.00% | 0.00% | 0.00% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | — | 0.00% | 0.00% | 0.00% | 0.00% | 0.02% | 0.03% | 0.03% | 0.00% | 0.40% | 0.00% | 0.00% | 0.20% |
| N1387spilt-KKH- | | | | | | | | | | | | | |
| Cas9-N1387-C and | | | | | | | | | | | | | |
| dSpCas9-N1387-N | | | | | | | | | | | | | |

| | Nucle- | | | | | | | | | | | | |
|---|---|---|---|---|---|---|---|---|---|---|---|---|---|
| Batch | otide | T | A | T | G | T | A | G | C | C | T | C | A |
| nogRNA- | A | 0.01% | 99.89% | 0.00% | 0.04% | 0.00% | 99.94% | 0.03% | 0.01% | 0.00% | 0.00% | 0.00% | 99.96% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | T | 99.94% | 0.00% | 99.98% | 0.02% | 99.98% | 0.00% | 0.03% | 0.02% | 0.01% | 99.99% | 0.02% | 0.00% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | C | 0.00% | 0.01% | 0.01% | 0.00% | 0.01% | 0.03% | 0.00% | 99.97% | 99.99% | 0.00% | 99.96% | 0.03% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | G | 0.00% | 0.05% | 0.00% | 99.93% | 0.00% | 0.02% | 99.93% | 0.00% | 0.00% | 0.00% | 0.01% | 0.01% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | — | 0.05% | 0.05% | 0.00% | 0.01% | 0.01% | 0.00% | 0.00% | 0.00% | 0.00% | 0.00% | 0.00% | 0.00% |
| A1343spilt-KKH- | | | | | | | | | | | | | |
| Cas9-A1343-N and | | | | | | | | | | | | | |
| dSpCas9-A1343-C | | | | | | | | | | | | | |
| nogRNA- | A | 0.00% | 99.86% | 0.02% | 0.04% | 0.01% | 99.91% | 0.02% | 0.01% | 0.01% | 0.00% | 0.02% | 99.91% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | T | 99.97% | 0.02% | 99.96% | 0.03% | 99.97% | 0.00% | 0.09% | 0.03% | 0.00% | 99.96% | 0.02% | 0.01% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | C | 0.01% | 0.03% | 0.02% | 0.01% | 0.01% | 0.04% | 0.00% | 99.96% | 99.98% | 0.02% | 99.96% | 0.04% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | G | 0.01% | 0.05% | 0.00% | 99.93% | 0.00% | 0.05% | 99.89 | 0.00% | 0.00% | 0.02% | 0.00% | 0.04% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | — | 0.01% | 0.05% | 0.00% | 0.00% | 0.00% | 0.00% | 0.00% | 0.00% | 0.00% | 0.00% | 0.00% | 0.00% |
| A1343spilt-KKH- | | | | | | | | | | | | | |
| Cas9-A1343-C and | | | | | | | | | | | | | |
| dSpCas9-A1343-N | | | | | | | | | | | | | |
| nogRNA- | A | 0.00% | 99.89% | 0.00% | 0.07% | 0.01% | 99.94% | 0.02% | 0.00% | 0.01% | 0.01% | 0.00% | 99.96% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | T | 99.96% | 0.01% | 99.97% | 0.03% | 99.98% | 0.00% | 0.06% | 0.00% | 0.02% | 99.98% | 0.01% | 0.00% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | C | 0.01% | 0.01% | 0.02% | 0.00% | 0.01% | 0.01% | 0.00% | 100.00% | 99.97% | 0.01% | 99.99% | 0.02% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | G | 0.00% | 0.03% | 0.00% | 99.89% | 0.00% | 0.04% | 99.92% | 0.00% | 0.00% | 0.00% | 0.00% | 0.02% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | — | 0.03% | 0.05% | 0.00% | 0.00% | 0.00% | 0.00% | 0.00% | 0.00% | 0.00% | 0.00% | 0.00% | 0.00% |
| G1322spilt-KKH- | | | | | | | | | | | | | |
| Cas9-G1322-N and | | | | | | | | | | | | | |
| dSpCas9-G1322-C | | | | | | | | | | | | | |
| nogRNA- | A | 0.01% | 99.88% | 0.00% | 0.08% | 0.01% | 99.93% | 0.02% | 0.00% | 0.00% | 0.00% | 0.00% | 99.95% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | T | 99.94% | 0.02% | 99.97% | 0.03% | 99.96% | 0.03% | 0.07% | 0.00% | 0.01% | 99.97% | 0.03% | 0.00% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | C | 0.00% | 0.03% | 0.02% | 0.01% | 0.02% | 0.02% | 0.00% | 99.99% | 99.99% | 0.01% | 99.96% | 0.03% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | G | 0.01% | 0.05% | 0.00% | 99.88% | 0.00% | 0.02% | 99.91% | 0.00% | 0.00% | 0.01% | 0.00% | 0.02% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | — | 0.04% | 0.02% | 0.01% | 0.00% | 0.00% | 0.00% | 0.00% | 0.00% | 0.00% | 0.00% | 0.00% | 0.00% |
| G1322spilt-KKH- | | | | | | | | | | | | | |
| Cas9-G1322-C and | | | | | | | | | | | | | |
| dSpCas9-G1322-N | | | | | | | | | | | | | |
| nogRNA- | A | 0.00% | 99.88% | 0.01% | 0.07% | 0.02% | 99.94% | 0.01 | 0.01% | 0.00% | 0.01% | 0.00% | 99.95% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | T | 99.92% | 0.02% | 99.97% | 0.03% | 99.97% | 0.00% | 0.05% | 0.00% | 0.00% | 99.98% | 0.01% | 0.01% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | C | 0.01% | 0.01% | 0.00% | 0.00% | 0.01% | 0.02% | 0.00% | 99.99% | 99.99% | 0.01% | 99.99% | 0.02% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | G | 0.01% | 0.05% | 0.00% | 99.90% | 0.00% | 0.04% | 99.94% | 0.00% | 0.00% | 0.00% | 0.00% | 0.02% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | — | 0.06% | 0.04% | 0.01% | 0.00% | 0.00% | 0.00% | 0.00% | 0.00% | 0.00% | 0.00% | 0.00% | 0.00% |
| G1333spilt-KKH- | | | | | | | | | | | | | |
| Cas9-G1333-N and | | | | | | | | | | | | | |
| dSpCas9-G1333-C | | | | | | | | | | | | | |
| nogRNA- | A | 0.00% | 99.87% | 0.00% | 0.05% | 0.01% | 99.96% | 0.00% | 0.00% | 0.00% | 0.00% | 0.00% | 99.96% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | T | 99.95% | 0.01% | 99.97% | 0.04% | 99.97% | 0.01% | 0.06% | 0.01% | 0.02% | 100.00% | 0.01% | 0.00% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | C | 0.00% | 0.01% | 0.00% | 0.00% | 0.01% | 0.01% | 0.00% | 99.99% | 99.98% | 0.00% | 99.98% | 0.02% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | G | 0.00% | 0.05% | 0.00% | 99.90 | 0.00% | 0.02% | 99.94% | 0.00% | 0.00% | 0.00% | 0.00% | 0.02% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | — | 0.05% | 0.06% | 0.02% | 0.00% | 0.00% | 0.00% | 0.00% | 0.00% | 0.00% | 0.00% | 0.00% | 0.00% |
| G1333spilt-KKH- | | | | | | | | | | | | | |
| Cas9-G1333-C and | | | | | | | | | | | | | |
| dSpCas9-G1333-N | | | | | | | | | | | | | |
| nogRNA- | A | 0.01% | 99.89% | 0.00% | 0.05% | 0.01% | 99.95% | 0.02% | 0.01% | 0.01% | 0.01% | 0.01% | 99.96% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | T | 99.93% | 0.02% | 99.96% | 0.03% | 99.95% | 0.00% | 0.04% | 0.01% | 0.01% | 99.98% | 0.02% | 0.01% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | C | 0.02% | 0.00% | 0.01% | 0.00% | 0.03% | 0.01% | 0.00% | 99.98% | 99.98% | 0.01% | 99.97% | 0.01% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | G | 0.00% | 0.06% | 0.00% | 99.92% | 0.00% | 0.03% | 99.94% | 0.00% | 0.00% | 0.00% | 0.00% | 0.02% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | — | 0.05% | 0.03% | 0.03% | 0.00% | 0.00% | 0.00% | 0.00% | 0.00% | 0.00% | 0.00% | 0.00% | 0.00% |
| G1371spilt-KKH- | | | | | | | | | | | | | |
| Cas9-G1371-N and | | | | | | | | | | | | | |
| dSpCas9-G1371-C | | | | | | | | | | | | | |
| nogRNA- | A | 0.00% | 99.87% | 0.00% | 0.05% | 0.01% | 99.95% | 0.01% | 0.00% | 0.00% | 0.01% | 0.00% | 99.93% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | T | 99.96% | 0.03% | 99.96% | 0.05% | 99.96% | 0.01% | 0.06% | 0.00% | 0.01% | 99.97% | 0.03% | 0.01% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | C | 0.00% | 0.01% | 0.01% | 0.01% | 0.02% | 0.01% | 0.00% | 100.00% | 99.99% | 0.01% | 99.96% | 0.03% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | G | 0.01% | 0.06% | 0.01% | 99.89% | 0.00% | 0.03% | 99.92% | 0.00% | 0.00% | 0.01% | 0.00% | 0.02% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | — | 0.02% | 0.03% | 0.02% | 0.00% | 0.00% | 0.00% | 0.00% | 0.00% | 0.00% | 0.00% | 0.00% | 0.00% |
| G1371spilt-KKH- | | | | | | | | | | | | | |
| Cas9-G1371-C and | | | | | | | | | | | | | |
| dSpCas9-G1371-N | | | | | | | | | | | | | |
| nogRNA- | A | 0.01% | 99.86% | 0.00% | 0.07% | 0.01% | 99.94% | 0.01% | 0.00% | 0.00% | 0.01% | 0.00% | 99.93% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | T | 99.96% | 0.01% | 99.96% | 0.01% | 99.97% | 0.01% | 0.05% | 0.00% | 0.02% | 99.99% | 0.01% | 0.01% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | C | 0.01% | 0.02% | 0.01% | 0.01% | 0.02% | 0.03% | 0.00% | 99.99% | 99.98% | 0.00% | 99.99% | 0.03% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | G | 0.00% | 0.04% | 0.00% | 99.91% | 0.00% | 0.02% | 99.95% | 0.00% | 0.00% | 0.00% | 0.00% | 0.03% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | — | 0.02% | 0.07% | 0.02% | 0.00% | 0.00% | 0.00% | 0.00% | 0.00% | 0.00% | 0.00% | 0.00% | 0.00% |
| G1397spilt-KKH- | | | | | | | | | | | | | |
| Cas9-G1397-N and | | | | | | | | | | | | | |
| dSpCas9-G1397-C | | | | | | | | | | | | | |
| nogRNA- | A | 0.00% | 99.87% | 0.00% | 0.05% | 0.02% | 99.95% | 0.01% | 0.00% | 0.01% | 0.01% | 0.00% | 99.96% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | T | 99.92% | 0.01% | 99.96% | 0.02% | 99.95% | 0.01% | 0.05% | 0.01% | 0.01% | 99.97% | 0.01% | 0.00% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | C | 0.02% | 0.01% | 0.02% | 0.01% | 0.02% | 0.01% | 0.00% | 99.98% | 99.98% | 0.01% | 99.99% | 0.01% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | G | 0.01% | 0.05% | 0.01% | 99.92% | 0.00% | 0.02% | 99.94% | 0.00% | 0.00% | 0.01% | 0.00% | 0.02% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | — | 0.06% | 0.06% | 0.01% | 0.00% | 0.01% | 0.00% | 0.00% | 0.00% | 0.00% | 0.00% | 0.00% | 0.00% |
| G1397spilt-KKH- | | | | | | | | | | | | | |
| Cas9-G1397-C and | | | | | | | | | | | | | |
| dSpCas9-G1397-N | | | | | | | | | | | | | |
| nogRNA- | A | 0.00% | 99.89% | 0.01% | 0.07% | 0.03% | 99.95% | 0.02% | 0.00% | 0.02% | 0.00% | 0.00% | 99.95% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | T | 99.97% | 0.01% | 99.97% | 0.02% | 99.97% | 0.01% | 0.06% | 0.02% | 0.01% | 99.97% | 0.02% | 0.02% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | C | 0.02% | 0.01% | 0.01% | 0.00% | 0.00% | 0.02% | 0.00% | 99.98% | 99.97% | 0.03% | 99.98% | 0.02% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | G | 0.00% | 0.05% | 0.00% | 99.92% | 0.00% | 0.02% | 99.92% | 0.00% | 0.00% | 0.00% | 0.00% | 0.02% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | — | 0.01% | 0.04% | 0.01% | 0.00% | 0.00% | 0.00% | 0.00% | 0.00% | 0.00% | 0.00% | 0.00% | 0.00% |
| N1357spilt-KKH- | | | | | | | | | | | | | |
| Cas9-N1357-N and | | | | | | | | | | | | | |
| dSpCas9-N1357-C | | | | | | | | | | | | | |
| nogRNA- | A | 0.01% | 99.85% | 0.01% | 0.05% | 0.01% | 99.92% | 0.01% | 0.00% | 0.01% | 0.00% | 0.01% | 99.93% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | T | 99.94% | 0.00% | 99.95% | 0.03% | 99.98% | 0.02% | 0.04% | 0.01% | 0.00% | 99.98% | 0.01% | 0.01% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | C | 0.01% | 0.04% | 0.02% | 0.00% | 0.01% | 0.04% | 0.01% | 99.99% | 99.99% | 0.02% | 99.97% | 0.04% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | G | 0.00% | 0.04% | 0.01% | 99.92% | 0.00% | 0.02% | 99.94% | 0.00% | 0.00% | 0.00% | 0.01% | 0.02% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | — | 0.04% | 0.07% | 0.01% | 0.00% | 0.00% | 0.00% | 0.00% | 0.00% | 0.00% | 0.00% | 0.00% | 0.00% |
| N1357spilt-KKH- | | | | | | | | | | | | | |
| Cas9-N1357-C and | | | | | | | | | | | | | |
| dSpCas9-N1357-N | | | | | | | | | | | | | |
| nogRNA- | A | 0.01% | 99.85% | 0.00% | 0.04% | 0.02% | 99.90% | 0.01% | 0.00% | 0.01% | 0.00% | 0.00% | 99.95% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | T | 99.94% | 0.01% | 99.96% | 0.02% | 99.96% | 0.01% | 0.07% | 0.01% | 0.00% | 99.96% | 0.03% | 0.00% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | C | 0.01% | 0.01% | 0.02% | 0.00% | 0.01% | 0.03% | 0.00% | 99.99% | 99.99% | 0.02% | 99.97% | 0.03% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | G | 0.01% | 0.06% | 0.00% | 99.94% | 0.00% | 0.06% | 99.92% | 0.00% | 0.00% | 0.01% | 0.00% | 0.02% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | — | 0.02% | 0.07% | 0.02% | 0.00% | 0.00% | 0.00% | 0.00% | 0.00% | 0.00% | 0.00% | 0.00% | 0.00% |
| N1387spilt-KKH- | | | | | | | | | | | | | |
| Cas9-N1387-N and | | | | | | | | | | | | | |
| dSpCas9-N1387-C | | | | | | | | | | | | | |
| nogRNA- | A | 0.00% | 99.86% | 0.01% | 0.04% | 0.03% | 99.92% | 0.01% | 0.00% | 0.00% | 0.00% | 0.00% | 99.94% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | T | 99.94% | 0.00% | 99.98% | 0.02% | 99.96% | 0.00% | 0.09% | 0.01% | 0.01% | 99.97% | 0.03% | 0.00% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | C | 0.01% | 0.02% | 0.01% | 0.00% | 0.01% | 0.03% | 0.00% | 99.99% | 99.99% | 0.01% | 99.96% | 0.04% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | G | 0.01% | 0.06% | 0.00% | 99.94% | 0.00% | 0.04% | 99.89% | 0.00% | 0.00% | 0.01% | 0.00% | 0.02% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | N | 0.00% | 0.00% | 0.00% | 0.00% | 0.00% | 0.00% | 0.00% | 0.00% | 0.00% | 0.00% | 0.00% | 0.00% |
| N1387spilt-KKH- | | | | | | | | | | | | | |
| Cas9-N1387-C and | | | | | | | | | | | | | |
| dSpCas9-N1387-N | | | | | | | | | | | | | |
| nogRNA- | — | 0.04% | 0.05% | 0.00% | 0.01% | 0.00% | 0.00% | 0.00% | 0.00% | 0.00% | 0.00% | 0.00% | 0.00% |
| N1387spilt-KKH- | | | | | | | | | | | | | |
| Cas9-N1387-C and | | | | | | | | | | | | | |
| dSpCas9-N1387-N | | | | | | | | | | | | | |

| Batch | Nucleotide | G | T | C | T | T | C | C | C | A |
|---|---|---|---|---|---|---|---|---|---|---|
| nogRNA-A1343spilt-KKH- | A | 0.01% | 0.00% | 0.00% | 0.00% | 0.00% | 0.01% | 0.02% | 0.01% | 99.97% |
| Cas9-A1343-N and | | | | | | | | | | |
| dSpCas9-A1343-C | | | | | | | | | | |
| nogRNA-A1343spilt-KKH- | T | 0.00% | 99.99% | 0.01% | 99.98% | 99.95% | 0.01% | 0.00% | 0.02% | 0.01% |
| Cas9-A1343-N and | | | | | | | | | | |
| dSpCas9-A1343-C | | | | | | | | | | |
| nogRNA-A1343spilt-KKH- | C | 0.00% | 0.00% | 99.99% | 0.00% | 0.02% | 99.98% | 99.98% | 99.98% | 0.00% |
| Cas9-A1343-N and | | | | | | | | | | |
| dSpCas9-A1343-C | | | | | | | | | | |
| nogRNA-A1343spilt-KKH- | G | 99.98% | 0.00% | 0.00% | 0.01% | 0.03% | 0.00% | 0.01% | 0.00% | 0.02% |
| Cas9-A1343-N and | | | | | | | | | | |
| dSpCas9-A1343-C | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | A | 0.00% | 0.01% | 0.00% | 0.00% | 0.00% | 0.00% | 0.01% | 0.00% | 99.95% |
| Cas9-A1343-C and | | | | | | | | | | |
| dSpCas9-A1343-N | | | | | | | | | | |
| nogRNA-A1343spilt-KKH- | T | 0.00% | 99.98% | 0.01% | 99.99% | 99.95% | 0.01% | 0.02% | 0.02% | 0.01% |
| Cas9-A1343-C and | | | | | | | | | | |
| dSpCas9-A1343-N | | | | | | | | | | |
| nogRNA-A1343spilt-KKH- | C | 0.00% | 0.01% | 99.99% | 0.00% | 0.02% | 99.99% | 99.97% | 99.98% | 0.02% |
| Cas9-A1343-C and | | | | | | | | | | |
| dSpCas9-A1343-N | | | | | | | | | | |
| nogRNA-A1343spilt-KKH- | G | 99.99% | 0.00% | 0.00% | 0.01% | 0.02% | 0.00% | 0.00% | 0.00% | 0.02% |
| Cas9-A1343-C and | | | | | | | | | | |
| dSpCas9-A1343-N | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-G1322spilt-KKH- | A | 0.00% | 0.01% | 0.00% | 0.00% | 0.00% | 0.00% | 0.01% | 0.01% | 99.95% |
| Cas9-G1322-N and | | | | | | | | | | |
| dSpCas9-G1322-C | | | | | | | | | | |
| nogRNA-G1322spilt-KKH- | T | 0.01% | 99.98% | 0.00% | 99.98% | 99.96% | 0.01% | 0.00% | 0.01% | 0.01% |
| Cas9-G1322-N and | | | | | | | | | | |
| dSpCas9-G1322-C | | | | | | | | | | |
| nogRNA-G1322spilt-KKH- | C | 0.00% | 0.01% | 100.00% | 0.02% | 0.02% | 99.99% | 99.99% | 99.97% | 0.01% |
| Cas9-G1322-N and | | | | | | | | | | |
| dSpCas9-G1322-C | | | | | | | | | | |
| nogRNA-G1322spilt-KKH- | G | 99.99% | 0.00% | 0.00% | 0.00% | 0.02% | 0.00% | 0.00% | 0.00% | 0.03% |
| Cas9-G1322-N and | | | | | | | | | | |
| dSpCas9-G1322-C | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | — | 0.00% | 0.00% | 0.00% | 0.00% | 0.00% | 0.00% | 0.00% | 0.01% | 0.00% |
| Cas9-G1322-N and | | | | | | | | | | |
| dSpCas9-G1322-C | | | | | | | | | | |
| nogRNA-G1322spilt-KKH- | A | 0.02% | 0.01% | 0.00% | 0.00% | 0.01% | 0.00% | 0.00% | 0.00% | 99.94% |
| Cas9-G1322-C and | | | | | | | | | | |
| dSpCas9-G1322-N | | | | | | | | | | |
| nogRNA-G1322spilt-KKH- | T | 0.01% | 99.98% | 0.00% | 99.97% | 99.95% | 0.01% | 0.01% | 0.01% | 0.01% |
| Cas9-G1322-C and | | | | | | | | | | |
| dSpCas9-G1322-N | | | | | | | | | | |
| nogRNA-G1322spilt-KKH- | C | 0.00% | 0.01% | 99.99% | 0.02% | 0.02% | 99.99% | 99.99% | 99.98% | 0.01% |
| Cas9-G1322-C and | | | | | | | | | | |
| dSpCas9-G1322-N | | | | | | | | | | |
| nogRNA-G1322spilt-KKH- | G | 99.97% | 0.00% | 0.00% | 0.00% | 0.02% | 0.00% | 0.00% | 0.00% | 0.04% |
| Cas9-G1322-C and | | | | | | | | | | |
| dSpCas9-G1322-N | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1333spilt-KKH- | A | 0.00% | 0.01% | 0.00% | 0.00% | 0.00% | 0.00% | 0.00% | 0.00% | 99.97% |
| Cas9-G1333-N and | | | | | | | | | | |
| dSpCas9-G1333-C | | | | | | | | | | |
| nogRNA-G1333spilt-KKH- | T | 0.00% | 99.97% | 0.00% | 100.00% | 99.96% | 0.01% | 0.00% | 0.02% | 0.00% |
| Cas9-G1333-N and | | | | | | | | | | |
| dSpCas9-G1333-C | | | | | | | | | | |
| nogRNA-G1333spilt-KKH- | C | 0.00% | 0.02% | 100.00% | 0.00% | 0.02% | 99.99% | 99.99% | 99.98% | 0.00% |
| Cas9-G1333-N and | | | | | | | | | | |
| dSpCas9-G1333-C | | | | | | | | | | |
| nogRNA-G1333spilt-KKH- | G | 99.99% | 0.00% | 0.00% | 0.00% | 0.02% | 0.00% | 0.00% | 0.00% | 0.03% |
| Cas9-G1333-N and | | | | | | | | | | |
| dSpCas9-G1333-C | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | A | 0.01% | 0.00% | 0.00% | 0.00% | 0.00% | 0.00% | 0.00% | 0.00% | 99.95% |
| Cas9-G1333-C and | | | | | | | | | | |
| dSpCas9-G1333-N | | | | | | | | | | |
| nogRNA-G1333spilt-KKH- | T | 0.00% | 99.96% | 0.02% | 99.97% | 99.97% | 0.01% | 0.00% | 0.02% | 0.00% |
| Cas9-G1333-C and | | | | | | | | | | |
| dSpCas9-G1333-N | | | | | | | | | | |
| nogRNA-G1333spilt-KKH- | C | 0.00% | 0.03% | 99.98% | 0.02% | 0.01% | 99.99% | 99.98% | 99.98% | 0.00% |
| Cas9-G1333-C and | | | | | | | | | | |
| dSpCas9-G1333-N | | | | | | | | | | |
| nogRNA-G1333spilt-KKH- | G | 99.98% | 0.00% | 0.00% | 0.01% | 0.01% | 0.00% | 0.01% | 0.00% | 0.04% |
| Cas9-G1333-C and | | | | | | | | | | |
| dSpCas9-G1333-N | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1371spilt-KKH- | A | 0.02% | 0.01% | 0.00% | 0.00% | 0.00% | 0.00% | 0.01% | 0.00% | 99.96% |
| Cas9-G1371-N and | | | | | | | | | | |
| dSpCas9-G1371-C | | | | | | | | | | |
| nogRNA-G1371spilt-KKH- | T | 0.01% | 99.98% | 0.00% | 99.97% | 99.95% | 0.01% | 0.01% | 0.01% | 0.00% |
| Cas9-G1371-N and | | | | | | | | | | |
| dSpCas9-G1371-C | | | | | | | | | | |
| nogRNA-G1371spilt-KKH- | C | 0.00% | 0.01% | 100.00% | 0.02% | 0.03% | 99.99% | 99.98% | 99.98% | 0.01% |
| Cas9-G1371-N and | | | | | | | | | | |
| dSpCas9-G1371-C | | | | | | | | | | |
| nogRNA-G1371spilt-KKH- | G | 99.97% | 0.00% | 0.00% | 0.01% | 0.02% | 0.00% | 0.00% | 0.00% | 0.03% |
| Cas9-G1371-N and | | | | | | | | | | |
| dSpCas9-G1371-C | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | — | 0.00% | 0.00% | 0.00% | 0.00% | 0.00% | 0.00% | 0.00% | 0.01% | 0.00% |
| Cas9-G1371-N and | | | | | | | | | | |
| dSpCas9-G1371-C | | | | | | | | | | |
| nogRNA-G1371spilt-KKH- | A | 0.00% | 0.00% | 0.01% | 0.00% | 0.00% | 0.01% | 0.01% | 0.00% | 99.95% |
| Cas9-G1371-C and | | | | | | | | | | |
| dSpCas9-G1371-N | | | | | | | | | | |
| nogRNA-G1371spilt-KKH- | T | 0.01% | 99.98% | 0.01% | 99.96% | 99.97% | 0.01% | 0.00% | 0.00% | 0.01% |
| Cas9-G1371-C and | | | | | | | | | | |
| dSpCas9-G1371-N | | | | | | | | | | |
| nogRNA-G1371spilt-KKH- | C | 0.00% | 0.02% | 99.98% | 0.02% | 0.01% | 99.99% | 99.99% | 99.99% | 0.01% |
| Cas9-G1371-C and | | | | | | | | | | |
| dSpCas9-G1371-N | | | | | | | | | | |
| nogRNA-G1371spilt-KKH- | G | 99.99% | 0.00% | 0.00% | 0.02% | 0.01% | 0.00% | 0.00% | 0.00% | 0.03% |
| Cas9-G1371-C and | | | | | | | | | | |
| dSpCas9-G1371-N | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1371spilt-KKH- | — | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1397spilt-KKH- | A | 0.00% | 0.01% | 0.00% | 0.00% | 0.00% | 0.00% | 0.01% | 0.02% | 99.97% |
| Cas9-G1397-N and | | | | | | | | | | |
| dSpCas9-G1397-C | | | | | | | | | | |
| nogRNA-G1397spilt-KKH- | T | 0.02% | 99.97% | 0.00% | 99.96% | 99.98% | 0.02% | 0.00% | 0.01% | 0.01% |
| Cas9-G1397-N and | | | | | | | | | | |
| dSpCas9-G1397-C | | | | | | | | | | |
| nogRNA-G1397spilt-KKH- | C | 0.00% | 0.02% | 99.99% | 0.03% | 0.02% | 99.98% | 99.98% | 99.95% | 0.00% |
| Cas9-G1397-N and | | | | | | | | | | |
| dSpCas9-G1397-C | | | | | | | | | | |
| nogRNA-G1397spilt-KKH- | G | 99.98% | 0.00% | 0.01% | 0.00% | 0.00% | 0.00% | 0.00% | 0.00% | 0.01% |
| Cas9-G1397-N and | | | | | | | | | | |
| dSpCas9-G1397-C | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | — | 0.00% | 0.00% | 0.00% | 0.00% | 0.00% | 0.00% | 0.00% | 0.02% | 0.00% |
| Cas9-G1397-N and | | | | | | | | | | |
| dSpCas9-G1397-C | | | | | | | | | | |
| nogRNA-G1397spilt-KKH- | A | 0.01% | 0.00% | 0.00% | 0.00% | 0.00% | 0.00% | 0.00% | 0.01% | 99.96% |
| Cas9-G1397-C and | | | | | | | | | | |
| dSpCas9-G1397-N | | | | | | | | | | |
| nogRNA-G1397spilt-KKH- | T | 0.00% | 99.98% | 0.01% | 99.98% | 99.95% | 0.02% | 0.01% | 0.00% | 0.01% |
| Cas9-G1397-C and | | | | | | | | | | |
| dSpCas9-G1397-N | | | | | | | | | | |
| nogRNA-G1397spilt-KKH- | C | 0.00% | 0.02% | 99.99% | 0.01% | 0.03% | 99.97% | 99.98% | 99.98% | 0.00% |
| Cas9-G1397-C and | | | | | | | | | | |
| dSpCas9-G1397-N | | | | | | | | | | |
| nogRNA-G1397spilt-KKH- | G | 99.99% | 0.00% | 0.00% | 0.01% | 0.02% | 0.00% | 0.01% | 0.00% | 0.02% |
| Cas9-G1397-C and | | | | | | | | | | |
| dSpCas9-G1397-N | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-G1397spilt-KKH- | — | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-N1357spilt-KKH- | A | 0.01% | 0.01% | 0.00% | 0.01% | 0.01% | 0.00% | 0.01% | 0.01% | 99.98% |
| Cas9-N1357-N and | | | | | | | | | | |
| dSpCas9-N1357-C | | | | | | | | | | |
| nogRNA-N1357spilt-KKH- | T | 0.00% | 99.97% | 0.00% | 99.98% | 99.97% | 0.01% | 0.01% | 0.01% | 0.00% |
| Cas9-N1357-N and | | | | | | | | | | |
| dSpCas9-N1357-C | | | | | | | | | | |
| nogRNA-N1357spilt-KKH- | C | 0.00% | 0.02% | 100.00% | 0.01% | 0.02% | 99.99% | 99.98% | 99.98% | 0.00% |
| Cas9-N1357-N and | | | | | | | | | | |
| dSpCas9-N1357-C | | | | | | | | | | |
| nogRNA-N1357spilt-KKH- | G | 99.98% | 0.00% | 0.00% | 0.01% | 0.01% | 0.00% | 0.00% | 0.00% | 0.02% |
| Cas9-N1357-N and | | | | | | | | | | |
| dSpCas9-N1357-C | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | A | 0.02% | 0.01% | 0.00% | 0.01% | 0.01% | 0.01% | 0.01% | 0.01% | 99.93% |
| Cas9-N1357-C and | | | | | | | | | | |
| dSpCas9-N1357-N | | | | | | | | | | |
| nogRNA-N1357spilt-KKH- | T | 0.00% | 99.98% | 0.01% | 99.97% | 99.97% | 0.01% | 0.00% | 0.02% | 0.00% |
| Cas9-N1357-C and | | | | | | | | | | |
| dSpCas9-N1357-N | | | | | | | | | | |
| nogRNA-N1357spilt-KKH- | C | 0.00% | 0.01% | 99.99% | 0.02% | 0.01% | 99.98% | 99.99% | 99.98% | 0.02% |
| Cas9-N1357-C and | | | | | | | | | | |
| dSpCas9-N1357-N | | | | | | | | | | |
| nogRNA-N1357spilt-KKH- | G | 99.98% | 0.00% | 0.00% | 0.01% | 0.01% | 0.00% | 0.00% | 0.00% | 0.04% |
| Cas9-N1357-C and | | | | | | | | | | |
| dSpCas9-N1357-N | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1387spilt-KKH- | A | 0.00% | 0.00% | 0.00% | 0.00% | 0.01% | 0.00% | 0.01% | 0.02% | 99.96% |
| Cas9-N1387-N and | | | | | | | | | | |
| dSpCas9-N1387-C | | | | | | | | | | |
| nogRNA-N1387spilt-KKH- | T | 0.00% | 99.98% | 0.01% | 99.98% | 99.96% | 0.00% | 0.00% | 0.01% | 0.01% |
| Cas9-N1387-N and | | | | | | | | | | |
| dSpCas9-N1387-C | | | | | | | | | | |
| nogRNA-N1387spilt-KKH- | C | 0.00% | 0.01% | 99.98% | 0.01% | 0.01% | 100.00% | 99.98% | 99.97% | 0.01% |
| Cas9-N1387-N and | | | | | | | | | | |
| dSpCas9-N1387-C | | | | | | | | | | |
| nogRNA-N1387spilt-KKH- | G | 100.00% | 0.00% | 0.00% | 0.01% | 0.02% | 0.00% | 0.00% | 0.00% | 0.03% |
| Cas9-N1387-N and | | | | | | | | | | |
| dSpCas9-N1387-C | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | — | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | A | 0.01% | 0.00% | 0.00% | 0.00% | 0.01% | 0.00% | 0.00% | 0.00% | 99.96% |
| Cas9-N1387-C and | | | | | | | | | | |
| dSpCas9-N1387-N | | | | | | | | | | |
| nogRNA-N1387spilt-KKH- | T | 0.00% | 100.00% | 0.00% | 99.97% | 99.95% | 0.01% | 0.01% | 0.00% | 0.01% |
| Cas9-N1387-C and | | | | | | | | | | |
| dSpCas9-N1387-N | | | | | | | | | | |
| nogRNA-N1387spilt-KKH- | C | 0.00% | 0.00% | 100.00% | 0.01% | 0.02% | 99.98% | 99.99% | 99.99% | 0.00% |
| Cas9-N1387-C and | | | | | | | | | | |
| dSpCas9-N1387-N | | | | | | | | | | |
| nogRNA-N1387spilt-KKH- | G | 99.99% | 0.00% | 0.00% | 0.01% | 0.02% | 0.00% | 0.00% | 0.00% | 0.03% |
| Cas9-N1387-C and | | | | | | | | | | |
| dSpCas9-N1387-N | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |
| nogRNA-N1387spilt-KKH- | — | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |

| Batch | Nucleotide | T | C | A | G | G | C | T | C |
|---|---|---|---|---|---|---|---|---|---|
| nogRNA-A1343spilt-KKH- | A | 0.00% | 0.00% | 99.98% | 0.00% | 0.03% | 0.00% | 0.00% | 0.01% |
| Cas9-A1343-N and | | | | | | | | | |
| dSpCas9-A1343-C | | | | | | | | | |
| nogRNA-A1343spilt-KKH- | T | 99.99% | 0.01% | 0.00% | 0.00% | 0.01% | 0.01% | 100.00% | 0.01% |
| Cas9-A1343-N and | | | | | | | | | |
| dSpCas9-A1343-C | | | | | | | | | |
| nogRNA-A1343spilt-KKH- | C | 0.00% | 99.99% | 0.00% | 0.00% | 0.00% | 99.99% | 0.00% | 99.98% |
| Cas9-A1343-N and | | | | | | | | | |
| dSpCas9-A1343-C | | | | | | | | | |
| nogRNA-A1343spilt-KKH- | G | 0.00% | 0.00% | 0.02% | 99.99% | 99.96% | 0.00% | 0.00% | 0.00% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | A | 0.01% | 0.01% | 99.95% | 0.01% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | T | 99.99% | 0.01% | 0.02% | 0.00% | 0.00% | 0.00% | 99.98% | 0.00% |
| Cas9-A1343-C and | | | | | | | | | |
| dSpCas9-A1343-N | | | | | | | | | |
| nogRNA-A1343spilt-KKH- | C | 0.00% | 99.97% | 0.03% | 0.00% | 0.00% | 100.00% | 0.02% | 99.99% |
| Cas9-A1343-C and | | | | | | | | | |
| dSpCas9-A1343-N | | | | | | | | | |
| nogRNA-A1343spilt-KKH- | G | 0.00% | 0.00% | 0.00% | 99.99% | 10.000% | 0.00% | 0.00% | 0.00% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-G1322spilt-KKH- | A | 0.01% | 0.01% | 99.94% | 0.00% | 0.01% | 0.00% | 0.00% | 0.01% |
| Cas9-G1322-N and | | | | | | | | | |
| dSpCas9-G1322-C | | | | | | | | | |
| nogRNA-G1322spilt-KKH- | T | 99.98% | 0.01% | 0.03% | 0.01% | 0.00% | 0.00% | 99.99% | 0.01% |
| Cas9-G1322-N and | | | | | | | | | |
| dSpCas9-G1322-C | | | | | | | | | |
| nogRNA-G1322spilt-KKH- | C | 0.00% | 99.98% | 0.00% | 0.00% | 0.00% | 100.00% | 0.00% | 99.99% |
| Cas9-G1322-N and | | | | | | | | | |
| dSpCas9-G1322-C | | | | | | | | | |
| nogRNA-G1322spilt-KKH- | G | 0.01% | 0.00% | 0.03% | 99.99% | 99.99% | 0.00% | 0.00% | 0.00% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | A | 0.00% | 0.00% | 99.96% | 0.00% | 0.01% | 0.00% | 0.00% | 0.00% |
| Cas9-G1322-C and | | | | | | | | | |
| dSpCas9-G1322-N | | | | | | | | | |
| nogRNA-G1322spilt-KKH- | T | 99.97% | 0.01% | 0.01% | 0.02% | 0.01% | 0.01% | 99.99% | 0.01% |
| Cas9-G1322-C and | | | | | | | | | |
| dSpCas9-G1322-N | | | | | | | | | |
| nogRNA-G1322spilt-KKH- | C | 0.01% | 99.99% | 0.02% | 0.00% | 0.00% | 99.99% | 0.00% | 99.99% |
| Cas9-G1322-C and | | | | | | | | | |
| dSpCas9-G1322-N | | | | | | | | | |
| nogRNA-G1322spilt-KKH- | G | 0.02% | 0.00% | 0.01% | 99.98% | 99.98% | 0.00% | 0.00% | 0.00% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1333spilt-KKH- | A | 0.00% | 0.00% | 99.95% | 0.01% | 0.01% | 0.00% | 0.00% | 0.00% |
| Cas9-G1333-N and | | | | | | | | | |
| dSpCas9-G1333-C | | | | | | | | | |
| nogRNA-G1333spilt-KKH- | T | 99.97% | 0.00% | 0.01% | 0.00% | 0.00% | 0.01% | 100.00% | 0.00% |
| Cas9-G1333-N and | | | | | | | | | |
| dSpCas9-G1333-C | | | | | | | | | |
| nogRNA-G1333spilt-KKH- | C | 0.02% | 100.00% | 0.00% | 0.00% | 0.00% | 99.98% | 0.00% | 99.99% |
| Cas9-G1333-N and | | | | | | | | | |
| dSpCas9-G1333-C | | | | | | | | | |
| nogRNA-G1333spilt-KKH- | G | 0.00% | 0.00% | 0.03% | 99.99% | 99.99% | 0.00% | 0.00% | 0.00% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | A | 0.00% | 0.01% | 99.95% | 0.02% | 0.01% | 0.00% | 0.01% | 0.00% |
| Cas9-G1333-C and | | | | | | | | | |
| dSpCas9-G1333-N | | | | | | | | | |
| nogRNA-G1333spilt-KKH- | T | 99.98% | 0.00% | 0.02% | 0.00% | 0.01% | 0.00% | 99.98% | 0.00% |
| Cas9-G1333-C and | | | | | | | | | |
| dSpCas9-G1333-N | | | | | | | | | |
| nogRNA-G1333spilt-KKH- | C | 0.01% | 99.98% | 0.00% | 0.00% | 0.00% | 100.00% | 0.01% | 100.00% |
| Cas9-G1333-C and | | | | | | | | | |
| dSpCas9-G1333-N | | | | | | | | | |
| nogRNA-G1333spilt-KKH- | G | 0.01% | 0.00% | 0.02% | 99.98% | 99.97% | 0.00% | 0.00% | 0.00% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1371spilt-KKH- | A | 0.00% | 0.01% | 99.97% | 0.01% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | T | 99.98% | 0.02% | 0.01% | 0.01% | 0.02% | 0.00% | 99.98% | 0.00% |
| Cas9-G1371-N and | | | | | | | | | |
| dSpCas9-G1371-C | | | | | | | | | |
| nogRNA-G1371spilt-KKH- | C | 0.01% | 99.97% | 0.00% | 0.00% | 0.00% | 99.99% | 0.01% | 100.00% |
| Cas9-G1371-N and | | | | | | | | | |
| dSpCas9-G1371-C | | | | | | | | | |
| nogRNA-G1371spilt-KKH- | G | 0.01% | 0.00% | 0.02% | 99.98% | 99.98% | 0.00% | 0.00% | 0.00% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | A | 0.01% | 0.00% | 99.94% | 0.00% | 0.01% | 0.00% | 0.00% | 0.01% |
| Cas9-G1371-C and | | | | | | | | | |
| dSpCas9-G1371-N | | | | | | | | | |
| nogRNA-G1371spilt-KKH- | T | 99.98% | 0.01% | 0.02% | 0.01% | 0.01% | 0.00% | 99.99% | 0.01% |
| Cas9-G1371-C and | | | | | | | | | |
| dSpCas9-G1371-N | | | | | | | | | |
| nogRNA-G1371spilt-KKH- | C | 0.00% | 99.98% | 0.01% | 0.00% | 0.00% | 99.99% | 0.01% | 99.99% |
| Cas9-G1371-C and | | | | | | | | | |
| dSpCas9-G1371-N | | | | | | | | | |
| nogRNA-G1371spilt-KKH- | G | 0.01% | 0.00% | 0.03% | 99.99% | 99.98% | 0.00% | 0.00% | 0.00% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1371spilt-KKH- | — | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1397spilt-KKH- | A | 0.01% | 0.00% | 99.94% | 0.00% | 0.01% | 0.00% | 0.01% | 0.01% |
| Cas9-G1397-N and | | | | | | | | | |
| dSpCas9-G1397-C | | | | | | | | | |
| nogRNA-G1397spilt-KKH- | T | 99.99% | 0.03% | 0.02% | 0.00% | 0.00% | 0.00% | 99.99% | 0.01% |
| Cas9-G1397-N and | | | | | | | | | |
| dSpCas9-G1397-C | | | | | | | | | |
| nogRNA-G1397spilt-KKH- | C | 0.00% | 99.96% | 0.00% | 0.00% | 0.00% | 100.00% | 0.00% | 99.98% |
| Cas9-G1397-N and | | | | | | | | | |
| dSpCas9-G1397-C | | | | | | | | | |
| nogRNA-G1397spilt-KKH- | G | 0.00% | 0.00% | 0.03% | 100.00% | 99.99% | 0.00% | 0.00% | 0.00% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | A | 0.00% | 0.01% | 99.95% | 0.01% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | T | 99.97% | 0.02% | 0.02% | 0.00% | 0.00% | 0.00% | 99.97% | 0.01% |
| Cas9-G1397-C and | | | | | | | | | |
| dSpCas9-G1397-N | | | | | | | | | |
| nogRNA-G1397spilt-KKH- | C | 0.01% | 99.97% | 0.00% | 0.00% | 0.00% | 100.00% | 0.01% | 99.99% |
| Cas9-G1397-C and | | | | | | | | | |
| dSpCas9-G1397-N | | | | | | | | | |
| nogRNA-G1397spilt-KKH- | G | 0.02% | 0.00% | 0.02% | 99.99% | 99.99% | 0.00% | 0.01% | 0.00% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-N1357spilt-KKH- | A | 0.00% | 0.01% | 99.96% | 0.01% | 0.01% | 0.00% | 0.00% | 0.00% |
| Cas9-N1357-N and | | | | | | | | | |
| dSpCas9-N1357-C | | | | | | | | | |
| nogRNA-N1357spilt-KKH- | T | 99.98% | 0.01% | 0.01% | 0.00% | 0.01% | 0.01% | 99.99% | 0.01% |
| Cas9-N1357-N and | | | | | | | | | |
| dSpCas9-N1357-C | | | | | | | | | |
| nogRNA-N1357spilt-KKH- | C | 0.00% | 99.98% | 0.01% | 0.00% | 0.00% | 99.99% | 0.00% | 99.99% |
| Cas9-N1357-N and | | | | | | | | | |
| dSpCas9-N1357-C | | | | | | | | | |
| nogRNA-N1357spilt-KKH- | G | 0.01% | 0.00% | 0.02% | 99.99% | 99.98% | 0.00% | 0.00% | 0.00% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | A | 0.01% | 0.01% | 99.93% | 0.00% | 0.01% | 0.00% | 0.00% | 0.00% |
| Cas9-N1357-C and | | | | | | | | | |
| dSpCas9-N1357-N | | | | | | | | | |
| nogRNA-N1357spilt-KKH- | T | 99.98% | 0.02% | 0.01% | 0.01% | 0.00% | 0.00% | 99.98% | 0.01% |
| Cas9-N1357-C and | | | | | | | | | |
| dSpCas9-N1357-N | | | | | | | | | |
| nogRNA-N1357spilt-KKH- | C | 0.01% | 99.97% | 0.03% | 0.00% | 0.00% | 99.99% | 0.01% | 99.99% |
| Cas9-N1357-C and | | | | | | | | | |
| dSpCas9-N1357-N | | | | | | | | | |
| nogRNA-N1357spilt-KKH- | G | 0.01% | 0.00% | 0.03% | 99.99% | 99.99% | 0.01% | 0.01% | 0.00% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1387spilt-KKH- | A | 0.00% | 0.00% | 99.95% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | T | 99.98% | 0.01% | 0.02% | 0.00% | 0.00% | 0.00% | 99.98% | 0.01% |
| Cas9-N1387-N and | | | | | | | | | |
| dSpCas9-N1387-C | | | | | | | | | |
| nogRNA-N1387spilt-KKH- | C | 0.01% | 99.98% | 0.01% | 0.00% | 0.00% | 99.98% | 0.02% | 99.99% |
| Cas9-N1387-N and | | | | | | | | | |
| dSpCas9-N1387-C | | | | | | | | | |
| nogRNA-N1387spilt-KKH- | G | 0.01% | 0.00% | 0.02% | 100.00% | 99.99% | 0.01% | 0.00% | 0.00% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | A | 0.00% | 0.00% | 99.96% | 0.01% | 0.01% | 0.00% | 0.00% | 0.00% |
| Cas9-N1387-C and | | | | | | | | | |
| dSpCas9-N1387-N | | | | | | | | | |
| nogRNA-N1387spilt-KKH- | T | 99.97% | 0.02% | 0.01% | 0.00% | 0.00% | 0.00% | 99.98% | 0.00% |
| Cas9-N1387-C and | | | | | | | | | |
| dSpCas9-N1387-N | | | | | | | | | |
| nogRNA-N1387spilt-KKH- | C | 0.02% | 99.97% | 0.01% | 0.00% | 0.00% | 100.00% | 0.01% | 100.00% |
| Cas9-N1387-C and | | | | | | | | | |
| dSpCas9-N1387-N | | | | | | | | | |
| nogRNA-N1387spilt-KKH- | G | 0.01% | 0.00% | 0.02% | 99.98% | 99.99% | 0.00% | 0.00% | 0.00% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |

| Batch | Nucleotide | T | C | A | G | C | T | C |
|---|---|---|---|---|---|---|---|---|
| nogRNA-A1343spilt-KKH- | A | 0.01% | 0.00% | 99.97% | 0.02% | 0.01% | 0.00% | 0.00% |
| Cas9-A1343-N and | | | | | | | | |
| dSpCas9-A1343-C | | | | | | | | |
| nogRNA-A1343spilt-KKH- | T | 99.97% | 0.03% | 0.00% | 0.00% | 0.01% | 99.99% | 0.02% |
| Cas9-A1343-N and | | | | | | | | |
| dSpCas9-A1343-C | | | | | | | | |
| nogRNA-A1343spilt-KKH- | C | 0.02% | 99.96% | 0.00% | 0.00% | 99.98% | 0.01% | 99.98% |
| Cas9-A1343-N and | | | | | | | | |
| dSpCas9-A1343-C | | | | | | | | |
| nogRNA-A1343spilt-KKH- | G | 0.00% | 0.00% | 0.02% | 99.98% | 0.00% | 0.00% | 0.00% |
| Cas9-A1343-N and | | | | | | | | |
| dSpCas9-A1343-C | | | | | | | | |
| nogRNA-A1343spilt-KKH- | N | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | — | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | A | 0.00% | 0.01% | 99.96% | 0.01% | 0.00% | 0.00% | 0.00% |
| Cas9-A1343-C and | | | | | | | | |
| dSpCas9-A1343-N | | | | | | | | |
| nogRNA-A1343spilt-KKH- | T | 99.97% | 0.02% | 0.00% | 0.00% | 0.01% | 99.98% | 0.00% |
| Cas9-A1343-C and | | | | | | | | |
| dSpCas9-A1343-N | | | | | | | | |
| nogRNA-A1343spilt-KKH- | C | 0.02% | 99.97% | 0.01% | 0.00% | 99.99% | 0.01% | 99.99% |
| Cas9-A1343-C and | | | | | | | | |
| dSpCas9-A1343-N | | | | | | | | |
| nogRNA-A1343spilt-KKH- | G | 0.00% | 0.00% | 0.03% | 99.98% | 0.00% | 0.00% | 0.00% |
| Cas9-A1343-C and | | | | | | | | |
| dSpCas9-A1343-N | | | | | | | | |
| nogRNA-A1343spilt-KKH- | N | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-A1343spilt-KKH- | — | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-G1322spilt-KKH- | A | 0.00% | 0.01% | 99.98% | 0.01% | 0.00% | 0.00% | 0.01% |
| Cas9-G1322-N and | | | | | | | | |
| dSpCas9-G1322-C | | | | | | | | |
| nogRNA-G1322spilt-KKH- | T | 99.98% | 0.01% | 0.01% | 0.00% | 0.00% | 99.98% | 0.02% |
| Cas9-G1322-N and | | | | | | | | |
| dSpCas9-G1322-C | | | | | | | | |
| nogRNA-G1322spilt-KKH- | C | 0.02% | 99.98% | 0.00% | 0.00% | 100.00% | 0.01% | 99.97% |
| Cas9-G1322-N and | | | | | | | | |
| dSpCas9-G1322-C | | | | | | | | |
| nogRNA-G1322spilt-KKH- | G | 0.00% | 0.00% | 0.01% | 99.98% | 0.00% | 0.01% | 0.00% |
| Cas9-G1322-N and | | | | | | | | |
| dSpCas9-G1322-C | | | | | | | | |
| nogRNA-G1322spilt-KKH- | N | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | — | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | A | 0.00% | 0.00% | 99.98% | 0.00% | 0.00% | 0.00% | 0.00% |
| Cas9-G1322-C and | | | | | | | | |
| dSpCas9-G1322-N | | | | | | | | |
| nogRNA-G1322spilt-KKH- | T | 99.97% | 0.01% | 0.00% | 0.01% | 0.00% | 99.98% | 0.03% |
| Cas9-G1322-C and | | | | | | | | |
| dSpCas9-G1322-N | | | | | | | | |
| nogRNA-G1322spilt-KKH- | C | 0.03% | 99.99% | 0.00% | 0.01% | 100.00% | 0.01% | 99.97% |
| Cas9-G1322-C and | | | | | | | | |
| dSpCas9-G1322-N | | | | | | | | |
| nogRNA-G1322spilt-KKH- | G | 0.00% | 0.00% | 0.02% | 99.98% | 0.00% | 0.01% | 0.00% |
| Cas9-G1322-C and | | | | | | | | |
| dSpCas9-G1322-N | | | | | | | | |
| nogRNA-G1322spilt-KKH- | N | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1322spilt-KKH- | — | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1333spilt-KKH- | A | 0.00% | 0.02% | 100.00% | 0.02% | 0.00% | 0.00% | 0.00% |
| Cas9-G1333-N and | | | | | | | | |
| dSpCas9-G1333-C | | | | | | | | |
| nogRNA-G1333spilt-KKH- | T | 99.99% | 0.01% | 0.00% | 0.00% | 0.00% | 100.00% | 0.02% |
| Cas9-G1333-N and | | | | | | | | |
| dSpCas9-G1333-C | | | | | | | | |
| nogRNA-G1333spilt-KKH- | C | 0.00% | 99.97% | 0.00% | 0.00% | 100.00% | 0.00% | 99.98% |
| Cas9-G1333-N and | | | | | | | | |
| dSpCas9-G1333-C | | | | | | | | |
| nogRNA-G1333spilt-KKH- | G | 0.00% | 0.00% | 0.00% | 99.98% | 0.00% | 0.00% | 0.00% |
| Cas9-G1333-N and | | | | | | | | |
| dSpCas9-G1333-C | | | | | | | | |
| nogRNA-G1333spilt-KKH- | N | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | — | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | A | 0.00% | 0.00% | 99.97% | 0.01% | 0.00% | 0.00% | 0.01% |
| Cas9-G1333-C and | | | | | | | | |
| dSpCas9-G1333-N | | | | | | | | |
| nogRNA-G1333spilt-KKH- | T | 99.98% | 0.01% | 0.01% | 0.00% | 0.00% | 100.00% | 0.01% |
| Cas9-G1333-C and | | | | | | | | |
| dSpCas9-G1333-N | | | | | | | | |
| nogRNA-G1333spilt-KKH- | C | 0.01% | 99.98% | 0.00% | 0.00% | 10.000% | 0.00% | 99.98% |
| Cas9-G1333-C and | | | | | | | | |
| dSpCas9-G1333-N | | | | | | | | |
| nogRNA-G1333spilt-KKH- | G | 0.00% | 0.00% | 0.02% | 99.99% | 0.00% | 0.00% | 0.00% |
| Cas9-G1333-C and | | | | | | | | |
| dSpCas9-G1333-N | | | | | | | | |
| nogRNA-G1333spilt-KKH- | N | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1333spilt-KKH- | — | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1371spilt-KKH- | A | 0.02% | 0.02% | 99.98% | 0.01% | 0.00% | 0.00% | 0.00% |
| Cas9-G1371-N and | | | | | | | | |
| dSpCas9-G1371-C | | | | | | | | |
| nogRNA-G1371spilt-KKH- | T | 99.98% | 0.01% | 0.00% | 0.01% | 0.01% | 99.99% | 0.02% |
| Cas9-G1371-N and | | | | | | | | |
| dSpCas9-G1371-C | | | | | | | | |
| nogRNA-G1371spilt-KKH- | C | 0.00% | 99.98% | 0.00% | 0.01% | 99.99% | 0.00% | 99.97% |
| Cas9-G1371-N and | | | | | | | | |
| dSpCas9-G1371-C | | | | | | | | |
| nogRNA-G1371spilt-KKH- | G | 0.00% | 0.00% | 0.02% | 99.98% | 0.00% | 0.00% | 0.00% |
| Cas9-G1371-N and | | | | | | | | |
| dSpCas9-G1371-C | | | | | | | | |
| nogRNA-G1371spilt-KKH- | N | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | — | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | A | 0.01% | 0.00% | 99.93% | 0.01% | 0.00% | 0.00% | 0.01% |
| Cas9-G1371-C and | | | | | | | | |
| dSpCas9-G1371-N | | | | | | | | |
| nogRNA-G1371spilt-KKH- | T | 99.99% | 0.01% | 0.01% | 0.00% | 0.01% | 99.98% | 0.03% |
| Cas9-G1371-C and | | | | | | | | |
| dSpCas9-G1371-N | | | | | | | | |
| nogRNA-G1371spilt-KKH- | C | 0.00% | 99.99% | 0.01% | 0.00% | 99.99% | 0.01% | 99.96% |
| Cas9-G1371-C and | | | | | | | | |
| dSpCas9-G1371-N | | | | | | | | |
| nogRNA-G1371spilt-KKH- | G | 0.01% | 0.00% | 0.06% | 99.99% | 0.00% | 0.01% | 0.00% |
| Cas9-G1371-C and | | | | | | | | |
| dSpCas9-G1371-N | | | | | | | | |
| nogRNA-G1371spilt-KKH- | N | 0.00% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1371spilt-KKH- | — | 0.00% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1397spilt-KKH- | A | 0.00% | 0.01% | 99.98% | 0.02% | 0.00% | 0.00% | 0.00% |
| Cas9-G1397-N and | | | | | | | | |
| dSpCas9-G1397-C | | | | | | | | |
| nogRNA-G1397spilt-KKH- | T | 99.99% | 0.03% | 0.00% | 0.00% | 0.01% | 99.99% | 0.01% |
| Cas9-G1397-N and | | | | | | | | |
| dSpCas9-G1397-C | | | | | | | | |
| nogRNA-G1397spilt-KKH- | C | 0.01% | 99.96% | 0.00% | 0.00% | 99.99% | 0.00% | 99.99% |
| Cas9-G1397-N and | | | | | | | | |
| dSpCas9-G1397-C | | | | | | | | |
| nogRNA-G1397spilt-KKH- | G | 0.00% | 0.00% | 0.02% | 99.98% | 0.00% | 0.00% | 0.00% |
| Cas9-G1397-N and | | | | | | | | |
| dSpCas9-G1397-C | | | | | | | | |
| nogRNA-G1397spilt-KKH- | N | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | — | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | A | 0.01% | 0.00% | 99.96% | 0.01% | 0.00% | 0.01% | 0.01% |
| Cas9-G1397-C and | | | | | | | | |
| dSpCas9-G1397-N | | | | | | | | |
| nogRNA-G1397spilt-KKH- | T | 99.98% | 0.01% | 0.00% | 0.02% | 0.00% | 99.96% | 0.03% |
| Cas9-G1397-C and | | | | | | | | |
| dSpCas9-G1397-N | | | | | | | | |
| nogRNA-G1397spilt-KKH- | C | 0.01% | 99.98% | 0.00% | 0.00% | 100.00% | 0.01% | 99.96% |
| Cas9-G1397-C and | | | | | | | | |
| dSpCas9-G1397-N | | | | | | | | |
| nogRNA-G1397spilt-KKH- | G | 0.00% | 0.01% | 0.04% | 99.97% | 0.00% | 0.01% | 0.00% |
| Cas9-G1397-C and | | | | | | | | |
| dSpCas9-G1397-N | | | | | | | | |
| nogRNA-G1397spilt-KKH- | N | 0.00% | 0.00% | 0.00% | 0.00% | 0.00% | 0.00% | 0.00% |
| Cas9-G1397-C and | | | | | | | | |
| dSpCas9-G1397-N | | | | | | | | |
| nogRNA-G1397spilt-KKH- | — | 0.00% | 0.00% | 0.00% | 0.00% | 0.00% | 0.00% | 0.00% |
| Cas9-G1397-C and | | | | | | | | |
| dSpCas9-G1397-N | | | | | | | | |
| nogRNA-N1357spilt-KKH- | A | 0.00% | 0.01% | 99.98% | 0.03% | 0.00% | 0.00% | 0.01% |
| Cas9-N1357-N and | | | | | | | | |
| dSpCas9-N1357-C | | | | | | | | |
| nogRNA-N1357spilt-KKH- | T | 99.98% | 0.02% | 0.00% | 0.00% | 0.00% | 99.99% | 0.01% |
| Cas9-N1357-N and | | | | | | | | |
| dSpCas9-N1357-C | | | | | | | | |
| nogRNA-N1357spilt-KKH- | C | 0.00% | 99.97% | 0.00% | 0.00% | 99.99% | 0.00% | 99.98% |
| Cas9-N1357-N and | | | | | | | | |
| dSpCas9-N1357-C | | | | | | | | |
| nogRNA-N1357spilt-KKH- | G | 0.00% | 0.00% | 0.02% | 99.97% | 0.00% | 0.01% | 0.00% |
| Cas9-N1357-N and | | | | | | | | |
| dSpCas9-N1357-C | | | | | | | | |
| nogRNA-N1357spilt-KKH- | N | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | — | 0.01% | 0.01% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | A | 0.01% | 0.00% | 99.93% | 0.01% | 0.01% | 0.00% | 0.01% |
| Cas9-N1357-C and | | | | | | | | |
| dSpCas9-N1357-N | | | | | | | | |
| nogRNA-N1357spilt-KKH- | T | 99.98% | 0.02% | 0.00% | 0.00% | 0.02% | 99.98% | 0.02% |
| Cas9-N1357-C and | | | | | | | | |
| dSpCas9-N1357-N | | | | | | | | |
| nogRNA-N1357spilt-KKH- | C | 0.01% | 99.97% | 0.02% | 0.00% | 99.98% | 0.01% | 99.98% |
| Cas9-N1357-C and | | | | | | | | |
| dSpCas9-N1357-N | | | | | | | | |
| nogRNA-N1357spilt-KKH- | G | 0.00% | 0.01% | 0.04% | 99.99% | 0.00% | 0.01% | 0.00% |
| Cas9-N1357-C and | | | | | | | | |
| dSpCas9-N1357-N | | | | | | | | |
| nogRNA-N1357spilt-KKH- | N | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1357spilt-KKH- | — | 0.01% | 0.01% | 0.00% | 0.00% | 0.00% | 0.00% | 0.00% |
| Cas9-N1357-C and | | | | | | | | |
| dSpCas9-N1357-N | | | | | | | | |
| nogRNA-N1387spilt-KKH- | A | 0.00% | 0.01% | 99.99% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | T | 99.98% | 0.00% | 0.00% | 0.00% | 0.00% | 99.99% | 0.04% |
| Cas9-N1387-N and | | | | | | | | |
| dSpCas9-N1387-C | | | | | | | | |
| nogRNA-N1387spilt-KKH- | C | 0.01% | 99.98% | 0.00% | 0.01% | 100.00% | 0.00% | 99.96% |
| Cas9-N1387-N and | | | | | | | | |
| dSpCas9-N1387-C | | | | | | | | |
| nogRNA-N1387spilt-KKH- | G | 0.00% | 0.00% | 0.00% | 99.99% | 0.00% | 0.01% | 0.00% |
| Cas9-N1387-N and | | | | | | | | |
| dSpCas9-N1387-C | | | | | | | | |
| nogRNA-N1387spilt-KKH- | N | 0.00% | 0.00% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | — | 0.00% | 0.00% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | A | 0.01% | 0.00% | 99.96% | 0.01% | 0.00% | 0.00% | 0.01% |
| Cas9-N1387-C and | | | | | | | | |
| dSpCas9-N1387-N | | | | | | | | |
| nogRNA-N1387spilt-KKH- | T | 99.99% | 0.02% | 0.00% | 0.00% | 0.02% | 99.99% | 0.02% |
| Cas9-N1387-C and | | | | | | | | |
| dSpCas9-N1387-N | | | | | | | | |
| nogRNA-N1387spilt-KKH- | C | 0.00% | 99.98% | 0.00% | 0.00% | 99.98% | 0.00% | 99.97% |
| Cas9-N1387-C and | | | | | | | | |
| dSpCas9-N1387-N | | | | | | | | |
| nogRNA-N1387spilt-KKH- | G | 0.00% | 0.00% | 0.04% | 99.98% | 0.00% | 0.01% | 0.00% |
| Cas9-N1387-C and | | | | | | | | |
| dSpCas9-N1387-N | | | | | | | | |
| nogRNA-N1387spilt-KKH- | N | 0.00% | 0.00% | 0.00% | 0.00% | 0.00% | 0.00% | 0.00% |
| Cas9-N1387-C and | | | | | | | | |
| dSpCas9-N1387-N | | | | | | | | |
| nogRNA-N1387spilt-KKH- | — | 0.00% | 0.00% | 0.00% | 0.00% | 0.00% | 0.00% | 0.00% |
| Cas9-N1387-C and | | | | | | | | |
| dSpCas9-N1387-N | | | | | | | | |

| Batch | Nucleotide | A | G | C | C | T | G | A | G | T |
|---|---|---|---|---|---|---|---|---|---|---|
| nogRNA-A1343spilt-KKH- | A | 99.97% | 0.02% | 0.01% | 0.00% | 0.01% | 0.00% | 99.96% | 0.01% | 0.00% |
| Cas9-A1343-N and | | | | | | | | | | |
| dSpCas9-A1343-C | | | | | | | | | | |
| nogRNA-A1343spilt-KKH- | T | 0.01% | 0.01% | 0.01% | 0.01% | 99.97% | 0.00% | 0.00% | 0.00% | 99.98% |
| Cas9-A1343-N and | | | | | | | | | | |
| dSpCas9-A1343-C | | | | | | | | | | |
| nogRNA-A1343spilt-KKH- | C | 0.00% | 0.01% | 99.98% | 99.99% | 0.01% | 0.01% | 0.01% | 0.00% | 0.02% |
| Cas9-A1343-N and | | | | | | | | | | |
| dSpCas9-A1343-C | | | | | | | | | | |
| nogRNA-A1343spilt-KKH- | G | 0.02% | 99.96% | 0.00% | 0.00% | 0.00% | 99.98% | 0.02% | 99.99% | 0.00% |
| Cas9-A1343-N and | | | | | | | | | | |
| dSpCas9-A1343-C | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | A | 99.95% | 0.01% | 0.00% | 0.01% | 0.00% | 0.01% | 99.94% | 0.00% | 0.01% |
| Cas9-A1343-C and | | | | | | | | | | |
| dSpCas9-A1343-N | | | | | | | | | | |
| nogRNA-A1343spilt-KKH- | T | 0.00% | 0.00% | 0.00% | 0.01% | 99.99% | 0.02% | 0.00% | 0.00% | 99.96% |
| Cas9-A1343-C and | | | | | | | | | | |
| dSpCas9-A1343-N | | | | | | | | | | |
| nogRNA-A1343spilt-KKH- | C | 0.03% | 0.00% | 100.00% | 99.98% | 0.01% | 0.00% | 0.04% | 0.00% | 0.02% |
| Cas9-A1343-C and | | | | | | | | | | |
| dSpCas9-A1343-N | | | | | | | | | | |
| nogRNA-A1343spilt-KKH- | G | 0.01% | 99.99% | 0.00% | 0.00% | 0.00% | 99.97% | 0.02% | 100.00% | 0.00% |
| Cas9-A1343-C and | | | | | | | | | | |
| dSpCas9-A1343-N | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-G1322spilt-KKH- | A | 99.98% | 0.01% | 0.00% | 0.00% | 0.01% | 0.01% | 99.95% | 0.00% | 0.02% |
| Cas9-G1322-N and | | | | | | | | | | |
| dSpCas9-G1322-C | | | | | | | | | | |
| nogRNA-G1322spilt-KKH- | T | 0.00% | 0.01% | 0.00% | 0.01% | 99.97% | 0.01% | 0.00% | 0.00% | 99.95% |
| Cas9-G1322-N and | | | | | | | | | | |
| dSpCas9-G1322-C | | | | | | | | | | |
| nogRNA-G1322spilt-KKH- | C | 0.00% | 0.00% | 99.99% | 99.98% | 0.02% | 0.00% | 0.02% | 0.00% | 0.01% |
| Cas9-G1322-N and | | | | | | | | | | |
| dSpCas9-G1322-C | | | | | | | | | | |
| nogRNA-G1322spilt-KKH- | G | 0.02% | 99.98% | 0.00% | 0.00% | 0.00% | 99.98% | 0.03% | 99.99% | 0.02% |
| Cas9-G1322-N and | | | | | | | | | | |
| dSpCas9-G1322-C | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | A | 99.98% | 0.00% | 0.00% | 0.00% | 0.01% | 0.03% | 99.97% | 0.01% | 0.01% |
| Cas9-G1322-C and | | | | | | | | | | |
| dSpCas9-G1322-N | | | | | | | | | | |
| nogRNA-G1322spilt-KKH- | T | 0.00% | 0.00% | 0.00% | 0.01% | 99.97% | 0.00% | 0.01% | 0.00% | 99.98% |
| Cas9-G1322-C and | | | | | | | | | | |
| dSpCas9-G1322-N | | | | | | | | | | |
| nogRNA-G1322spilt-KKH- | C | 0.01% | 0.00% | 99.99% | 99.99% | 0.02% | 0.00% | 0.01% | 0.00% | 0.00% |
| Cas9-G1322-C and | | | | | | | | | | |
| dSpCas9-G1322-N | | | | | | | | | | |
| nogRNA-G1322spilt-KKH- | G | 0.01% | 99.99% | 0.00% | 0.00% | 0.00% | 99.97% | 0.01% | 99.99% | 0.00% |
| Cas9-G1322-C and | | | | | | | | | | |
| dSpCas9-G1322-N | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1333spilt-KKH- | A | 99.99% | 0.01% | 0.01% | 0.00% | 0.00% | 0.03% | 99.99% | 0.00% | 0.00% |
| Cas9-G1333-N and | | | | | | | | | | |
| dSpCas9-G1333-C | | | | | | | | | | |
| nogRNA-G1333spilt-KKH- | T | 0.00% | 0.00% | 0.01% | 0.02% | 100.00% | 0.01% | 0.00% | 0.00% | 99.97% |
| Cas9-G1333-N and | | | | | | | | | | |
| dSpCas9-G1333-C | | | | | | | | | | |
| nogRNA-G1333spilt-KKH- | C | 0.00% | 0.01% | 99.98% | 99.98% | 0.00% | 0.00% | 0.00% | 0.00% | 0.02% |
| Cas9-G1333-N and | | | | | | | | | | |
| dSpCas9-G1333-C | | | | | | | | | | |
| nogRNA-G1333spilt-KKH- | G | 0.01% | 99.98% | 0.00% | 0.00% | 0.00% | 99.96% | 0.01% | 100.00% | 0.01% |
| Cas9-G1333-N and | | | | | | | | | | |
| dSpCas9-G1333-C | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | A | 99.97% | 0.00% | 0.00% | 0.01% | 0.02% | 0.01% | 99.95% | 0.00% | 0.01% |
| Cas9-G1333-C and | | | | | | | | | | |
| dSpCas9-G1333-N | | | | | | | | | | |
| nogRNA-G1333spilt-KKH- | T | 0.01% | 0.01% | 0.00% | 0.02% | 99.95% | 0.01% | 0.02% | 0.00% | 99.96% |
| Cas9-G1333-C and | | | | | | | | | | |
| dSpCas9-G1333-N | | | | | | | | | | |
| nogRNA-G1333spilt-KKH- | C | 0.00% | 0.00% | 100.00% | 99.96% | 0.03% | 0.00% | 0.00% | 0.00% | 0.02% |
| Cas9-G1333-C and | | | | | | | | | | |
| dSpCas9-G1333-N | | | | | | | | | | |
| nogRNA-G1333spilt-KKH- | G | 0.02% | 99.99% | 0.00% | 0.00% | 0.00% | 99.98% | 0.01% | 99.99% | 0.00% |
| Cas9-G1333-C and | | | | | | | | | | |
| dSpCas9-G1333-N | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1371spilt-KKH- | A | 99.96% | 0.02% | 0.00% | 0.00% | 0.00% | 0.01% | 99.98% | 0.00% | 0.01% |
| Cas9-G1371-N and | | | | | | | | | | |
| dSpCas9-G1371-C | | | | | | | | | | |
| nogRNA-G1371spilt-KKH- | T | 0.01% | 0.00% | 0.00% | 0.01% | 99.98% | 0.01% | 0.00% | 0.00% | 99.97% |
| Cas9-G1371-N and | | | | | | | | | | |
| dSpCas9-G1371-C | | | | | | | | | | |
| nogRNA-G1371spilt-KKH- | C | 0.01% | 0.00% | 100.00% | 99.98% | 0.01% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | G | 0.02% | 99.98% | 0.00% | 0.00% | 0.01% | 99.98% | 0.02% | 100.00% | 0.02% |
| Cas9-G1371-N and | | | | | | | | | | |
| dSpCas9-G1371-C | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | — | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | A | 99.99% | 0.01% | 0.00% | 0.00% | 0.00% | 0.00% | 99.96% | 0.01% | 0.00% |
| Cas9-G1371-C and | | | | | | | | | | |
| dSpCas9-G1371-N | | | | | | | | | | |
| nogRNA-G1371spilt-KKH- | T | 0.00% | 0.01% | 0.01% | 0.02% | 99.96% | 0.01% | 0.01% | 0.02% | 99.95% |
| Cas9-G1371-C and | | | | | | | | | | |
| dSpCas9-G1371-N | | | | | | | | | | |
| nogRNA-G1371spilt-KKH- | C | 0.00% | 0.00% | 99.98% | 99.98% | 0.03% | 0.00% | 0.00% | 0.00% | 0.02% |
| Cas9-G1371-C and | | | | | | | | | | |
| dSpCas9-G1371-N | | | | | | | | | | |
| nogRNA-G1371spilt-KKH- | G | 0.01% | 99.98% | 0.01% | 0.00% | 0.00% | 99.98% | 0.02% | 99.97% | 0.01% |
| Cas9-G1371-C and | | | | | | | | | | |
| dSpCas9-G1371-N | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1371spilt-KKH- | — | 0.00% | 0.00% | 0.00% | 0.00% | 0.00% | 0.01% | 0.01% | 0.01% | 0.01% |
| Cas9-G1371-C and | | | | | | | | | | |
| dSpCas9-G1371-N | | | | | | | | | | |
| nogRNA-G1397spilt-KKH- | A | 99.98% | 0.00% | 0.01% | 0.01% | 0.00% | 0.00% | 99.83% | 0.00% | 0.00% |
| Cas9-G1397-N and | | | | | | | | | | |
| dSpCas9-G1397-C | | | | | | | | | | |
| nogRNA-G1397spilt-KKH- | T | 0.01% | 0.01% | 0.00% | 0.00% | 99.83% | 0.00% | 0.00% | 0.00% | 99.84% |
| Cas9-G1397-N and | | | | | | | | | | |
| dSpCas9-G1397-C | | | | | | | | | | |
| nogRNA-G1397spilt-KKH- | C | 0.00% | 0.00% | 99.85% | 99.85% | 0.02% | 0.00% | 0.01% | 0.00% | 0.00% |
| Cas9-G1397-N and | | | | | | | | | | |
| dSpCas9-G1397-C | | | | | | | | | | |
| nogRNA-G1397spilt-KKH- | G | 0.01% | 99.99% | 0.01% | 0.00% | 0.00% | 99.86% | 0.02% | 99.86% | 0.01% |
| Cas9-G1397-N and | | | | | | | | | | |
| dSpCas9-G1397-C | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | — | 0.00% | 0.00% | 0.13% | 0.13% | 0.14% | 0.14% | 0.14% | 0.14% | 0.14% |
| Cas9-G1397-N and | | | | | | | | | | |
| dSpCas9-G1397-C | | | | | | | | | | |
| nogRNA-G1397spilt-KKH- | A | 99.98% | 0.01% | 0.00% | 0.00% | 0.01% | 0.01% | 99.96% | 0.01% | 0.01% |
| Cas9-G1397-C and | | | | | | | | | | |
| dSpCas9-G1397-N | | | | | | | | | | |
| nogRNA-G1397spilt-KKH- | T | 0.00% | 0.01% | 0.01% | 0.02% | 99.96% | 0.01% | 0.00% | 0.00% | 99.97% |
| Cas9-G1397-C and | | | | | | | | | | |
| dSpCas9-G1397-N | | | | | | | | | | |
| nogRNA-G1397spilt-KKH- | C | 0.00% | 0.00% | 99.99% | 99.98% | 0.02% | 0.00% | 0.01% | 0.00% | 0.02% |
| Cas9-G1397-C and | | | | | | | | | | |
| dSpCas9-G1397-N | | | | | | | | | | |
| nogRNA-G1397spilt-KKH- | G | 0.02% | 99.99% | 0.00% | 0.00% | 0.00% | 99.98% | 0.02% | 99.99% | 0.01% |
| Cas9-G1397-C and | | | | | | | | | | |
| dSpCas9-G1397-N | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-G1397spilt-KKH- | — | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-N1357spilt-KKH- | A | 99.96% | 0.02% | 0.00% | 0.01% | 0.00% | 0.01% | 99.96% | 0.00% | 0.01% |
| Cas9-N1357-N and | | | | | | | | | | |
| dSpCas9-N1357-C | | | | | | | | | | |
| nogRNA-N1357spilt-KKH- | T | 0.01% | 0.00% | 0.00% | 0.00% | 99.98% | 0.01% | 0.00% | 0.00% | 99.96% |
| Cas9-N1357-N and | | | | | | | | | | |
| dSpCas9-N1357-C | | | | | | | | | | |
| nogRNA-N1357spilt-KKH- | C | 0.01% | 0.00% | 100.00% | 99.98% | 0.01% | 0.00% | 0.00% | 0.00% | 0.01% |
| Cas9-N1357-N and | | | | | | | | | | |
| dSpCas9-N1357-C | | | | | | | | | | |
| nogRNA-N1357spilt-KKH- | G | 0.02% | 99.98% | 0.00% | 0.00% | 0.00% | 99.97% | 0.03% | 99.99% | 0.02% |
| Cas9-N1357-N and | | | | | | | | | | |
| dSpCas9-N1357-C | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | A | 99.92% | 0.01% | 0.00% | 0.00% | 0.00% | 0.01% | 99.93% | 0.01% | 0.01% |
| Cas9-N1357-C and | | | | | | | | | | |
| dSpCas9-N1357-N | | | | | | | | | | |
| nogRNA-N1357spilt-KKH- | T | 0.00% | 0.01% | 0.01% | 0.00% | 99.99% | 0.02% | 0.01% | 0.01% | 99.97% |
| Cas9-N1357-C and | | | | | | | | | | |
| dSpCas9-N1357-N | | | | | | | | | | |
| nogRNA-N1357spilt-KKH- | C | 0.05% | 0.00% | 99.99% | 10.000% | 0.01% | 0.00% | 0.04% | 0.00% | 0.02% |
| Cas9-N1357-C and | | | | | | | | | | |
| dSpCas9-N1357-N | | | | | | | | | | |
| nogRNA-N1357spilt-KKH- | G | 0.03% | 99.99% | 0.00% | 0.00% | 0.00% | 99.97% | 0.02% | 99.99% | 0.01% |
| Cas9-N1357-C and | | | | | | | | | | |
| dSpCas9-N1357-N | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1387spilt-KKH- | A | 99.98% | 0.01% | 0.00% | 0.00% | 0.00% | 0.01% | 99.96% | 0.00% | 0.01% |
| Cas9-N1387-N and | | | | | | | | | | |
| dSpCas9-N1387-C | | | | | | | | | | |
| nogRNA-N1387spilt-KKH- | T | 0.00% | 0.00% | 0.00% | 0.00% | 99.94% | 0.01% | 0.00% | 0.00% | 99.96% |
| Cas9-N1387-N and | | | | | | | | | | |
| dSpCas9-N1387-C | | | | | | | | | | |
| nogRNA-N1387spilt-KKH- | C | 0.00% | 0.01% | 99.98% | 99.99% | 0.04% | 0.00% | 0.00% | 0.00% | 0.01% |
| Cas9-N1387-N and | | | | | | | | | | |
| dSpCas9-N1387-C | | | | | | | | | | |
| nogRNA-N1387spilt-KKH- | G | 0.02% | 99.97% | 0.00% | 0.00% | 0.00% | 99.97% | 0.02% | 99.98% | 0.00% |
| Cas9-N1387-N and | | | | | | | | | | |
| dSpCas9-N1387-C | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | — | 0.00% | 0.00% | 0.00% | 0.00% | 0.01% | 0.01% | 0.01% | 0.01% | 0.01% |
| Cas9-N1387-N and | | | | | | | | | | |
| dSpCas9-N1387-C | | | | | | | | | | |
| nogRNA-N1387spilt-KKH- | A | 99.98% | 0.00% | 0.01% | 0.00% | 0.01% | 0.02% | 99.97% | 0.00% | 0.00% |
| Cas9-N1387-C and | | | | | | | | | | |
| dSpCas9-N1387-N | | | | | | | | | | |
| nogRNA-N1387spilt-KKH- | T | 0.00% | 0.00% | 0.01% | 0.01% | 99.96% | 0.01% | 0.00% | 0.00% | 99.96% |
| Cas9-N1387-C and | | | | | | | | | | |
| dSpCas9-N1387-N | | | | | | | | | | |
| nogRNA-N1387spilt-KKH- | C | 0.00% | 0.00% | 99.98% | 99.99% | 0.03% | 0.00% | 0.01% | 0.00% | 0.04% |
| Cas9-N1387-C and | | | | | | | | | | |
| dSpCas9-N1387-N | | | | | | | | | | |
| nogRNA-N1387spilt-KKH- | G | 0.02% | 100.00% | 0.00% | 0.00% | 0.00% | 99.97% | 0.02% | 100.00% | 0.00% |
| Cas9-N1387-C and | | | | | | | | | | |
| dSpCas9-N1387-N | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |
| nogRNA-N1387spilt-KKH- | — | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |

| Batch | Nucleotide | G | T | T | G | A | G | G | C |
|---|---|---|---|---|---|---|---|---|---|
| nogRNA-A1343spilt-KKH- | A | 0.00% | 0.01% | 0.00% | 0.02% | 99.98% | 0.03% | 0.02% | 0.00% |
| Cas9-A1343-N and | | | | | | | | | |
| dSpCas9-A1343-C | | | | | | | | | |
| nogRNA-A1343spilt-KKH- | T | 0.01% | 99.97% | 99.98% | 0.01% | 0.00% | 0.01% | 0.00% | 0.01% |
| Cas9-A1343-N and | | | | | | | | | |
| dSpCas9-A1343-C | | | | | | | | | |
| nogRNA-A1343spilt-KKH- | C | 0.01% | 0.00% | 0.01% | 0.00% | 0.01% | 0.00% | 0.00% | 99.98% |
| Cas9-A1343-N and | | | | | | | | | |
| dSpCas9-A1343-C | | | | | | | | | |
| nogRNA-A1343spilt-KKH- | G | 99.98% | 0.02% | 0.01% | 99.97% | 0.01% | 99.95% | 99.97% | 0.00% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.00% | 0.00% | 0.00% | 0.00% | 0.00% | 0.01% | 0.00% | 0.00% |
| Cas9-A1343-N and | | | | | | | | | |
| dSpCas9-A1343-C | | | | | | | | | |
| nogRNA-A1343spilt-KKH- | A | 0.00% | 0.00% | 0.00% | 0.01% | 99.97% | 0.02% | 0.01% | 0.00% |
| Cas9-A1343-C and | | | | | | | | | |
| dSpCas9-A1343-N | | | | | | | | | |
| nogRNA-A1343spilt-KKH- | T | 0.01% | 99.99% | 99.98% | 0.00% | 0.00% | 0.00% | 0.00% | 0.03% |
| Cas9-A1343-C and | | | | | | | | | |
| dSpCas9-A1343-N | | | | | | | | | |
| nogRNA-A1343spilt-KKH- | C | 0.00% | 0.00% | 0.01% | 0.00% | 0.02% | 0.00% | 0.00% | 99.97% |
| Cas9-A1343-C and | | | | | | | | | |
| dSpCas9-A1343-N | | | | | | | | | |
| nogRNA-A1343spilt-KKH- | G | 99.98% | 0.01% | 0.01% | 99.98% | 0.00% | 99.97% | 99.98% | 0.00% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-G1322spilt-KKH- | A | 0.00% | 0.01% | 0.01% | 0.02% | 99.97% | 0.03% | 0.01% | 0.00% |
| Cas9-G1322-N and | | | | | | | | | |
| dSpCas9-G1322-C | | | | | | | | | |
| nogRNA-G1322spilt-KKH- | T | 0.01% | 99.97% | 99.99% | 0.00% | 0.01% | 0.01% | 0.01% | 0.01% |
| Cas9-G1322-N and | | | | | | | | | |
| dSpCas9-G1322-C | | | | | | | | | |
| nogRNA-G1322spilt-KKH- | C | 0.00% | 0.01% | 0.00% | 0.00% | 0.00% | 0.00% | 0.00% | 99.98% |
| Cas9-G1322-N and | | | | | | | | | |
| dSpCas9-G1322-C | | | | | | | | | |
| nogRNA-G1322spilt-KKH- | G | 99.99% | 0.01% | 0.00% | 99.96% | 0.02% | 99.96% | 99.98% | 0.01% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.00% | 0.00% | 0.00% | 0.01% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | A | 0.00% | 0.00% | 0.00% | 0.01% | 99.95% | 0.01% | 0.02% | 0.01% |
| Cas9-G1322-C and | | | | | | | | | |
| dSpCas9-G1322-N | | | | | | | | | |
| nogRNA-G1322spilt-KKH- | T | 0.02% | 99.95% | 99.96% | 0.00% | 0.00% | 0.01% | 0.01% | 0.02% |
| Cas9-G1322-C and | | | | | | | | | |
| dSpCas9-G1322-N | | | | | | | | | |
| nogRNA-G1322spilt-KKH- | C | 0.00% | 0.03% | 0.03% | 0.00% | 0.01% | 0.00% | 0.00% | 99.97% |
| Cas9-G1322-C and | | | | | | | | | |
| dSpCas9-G1322-N | | | | | | | | | |
| nogRNA-G1322spilt-KKH- | G | 99.97% | 0.02% | 0.00% | 99.97% | 0.01% | 99.96% | 99.97% | 0.00% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.00% | 0.00% | 0.00% | 0.01% | 0.02% | 0.02% | 0.00% | 0.00% |
| Cas9-G1322-C and | | | | | | | | | |
| dSpCas9-G1322-N | | | | | | | | | |
| nogRNA-G1333spilt-KKH- | A | 0.00% | 0.01% | 0.00% | 0.02% | 99.98% | 0.01% | 0.01% | 0.00% |
| Cas9-G1333-N and | | | | | | | | | |
| dSpCas9-G1333-C | | | | | | | | | |
| nogRNA-G1333spilt-KKH- | T | 0.01% | 99.96% | 99.99% | 0.00% | 0.00% | 0.00% | 0.00% | 0.01% |
| Cas9-G1333-N and | | | | | | | | | |
| dSpCas9-G1333-C | | | | | | | | | |
| nogRNA-G1333spilt-KKH- | C | 0.00% | 0.01% | 0.01% | 0.00% | 0.00% | 0.00% | 0.00% | 99.99% |
| Cas9-G1333-N and | | | | | | | | | |
| dSpCas9-G1333-C | | | | | | | | | |
| nogRNA-G1333spilt-KKH- | G | 99.98% | 0.02% | 0.00% | 99.95% | 0.01% | 99.98% | 99.98% | 0.00% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.00% | 0.00% | 0.00% | 0.02% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | A | 0.00% | 0.01% | 0.01% | 0.01% | 99.95% | 0.03% | 0.01% | 0.00% |
| Cas9-G1333-C and | | | | | | | | | |
| dSpCas9-G1333-N | | | | | | | | | |
| nogRNA-G1333spilt-KKH- | T | 0.00% | 99.96% | 99.97% | 0.01% | 0.02% | 0.02% | 0.00% | 0.01% |
| Cas9-G1333-C and | | | | | | | | | |
| dSpCas9-G1333-N | | | | | | | | | |
| nogRNA-G1333spilt-KKH- | C | 0.00% | 0.02% | 0.02% | 0.00% | 0.01% | 0.00% | 0.01% | 99.98% |
| Cas9-G1333-C and | | | | | | | | | |
| dSpCas9-G1333-N | | | | | | | | | |
| nogRNA-G1333spilt-KKH- | G | 99.99% | 0.00% | 0.00% | 99.98% | 0.02% | 99.95% | 99.97% | 0.01% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1371spilt-KKH- | A | 0.02% | 0.00% | 0.00% | 0.02% | 99.95% | 0.02% | 0.00% | 0.01% |
| Cas9-G1371-N and | | | | | | | | | |
| dSpCas9-G1371-C | | | | | | | | | |
| nogRNA-G1371spilt-KKH- | T | 0.02% | 99.98% | 99.99% | 0.00% | 0.01% | 0.00% | 0.01% | 0.02% |
| Cas9-G1371-N and | | | | | | | | | |
| dSpCas9-G1371-C | | | | | | | | | |
| nogRNA-G1371spilt-KKH- | C | 0.00% | 0.02% | 0.01% | 0.00% | 0.00% | 0.00% | 0.00% | 99.97% |
| Cas9-G1371-N and | | | | | | | | | |
| dSpCas9-G1371-C | | | | | | | | | |
| nogRNA-G1371spilt-KKH- | G | 99.97% | 0.00% | 0.00% | 99.97% | 0.04% | 99.97% | 99.99% | 0.00% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.00% | 0.00% | 0.00% | 0.01% | 0.01% | 0.01% | 0.00% | 0.00% |
| Cas9-G1371-N and | | | | | | | | | |
| dSpCas9-G1371-C | | | | | | | | | |
| nogRNA-G1371spilt-KKH- | A | 0.00% | 0.01% | 0.00% | 0.01% | 99.95% | 0.01% | 0.01% | 0.00% |
| Cas9-G1371-C and | | | | | | | | | |
| dSpCas9-G1371-N | | | | | | | | | |
| nogRNA-G1371spilt-KKH- | T | 0.00% | 99.95% | 99.98% | 0.00% | 0.01% | 0.01% | 0.01% | 0.01% |
| Cas9-G1371-C and | | | | | | | | | |
| dSpCas9-G1371-N | | | | | | | | | |
| nogRNA-G1371spilt-KKH- | C | 0.00% | 0.02% | 0.01% | 0.00% | 0.00% | 0.00% | 0.00% | 99.98% |
| Cas9-G1371-C and | | | | | | | | | |
| dSpCas9-G1371-N | | | | | | | | | |
| nogRNA-G1371spilt-KKH- | G | 99.99% | 0.01% | 0.00% | 99.97% | 0.02% | 99.97% | 99.98% | 0.00% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1371spilt-KKH- | — | 0.01% | 0.01% | 0.01% | 0.01% | 0.01% | 0.01% | 0.01% | 0.01% |
| Cas9-G1371-C and | | | | | | | | | |
| dSpCas9-G1371-N | | | | | | | | | |
| nogRNA-G1397spilt-KKH- | A | 0.02% | 0.01% | 0.00% | 0.00% | 99.85% | 0.01% | 0.01% | 0.00% |
| Cas9-G1397-N and | | | | | | | | | |
| dSpCas9-G1397-C | | | | | | | | | |
| nogRNA-G1397spilt-KKH- | T | 0.01% | 99.83% | 99.83% | 0.00% | 0.00% | 0.00% | 0.01% | 0.00% |
| Cas9-G1397-N and | | | | | | | | | |
| dSpCas9-G1397-C | | | | | | | | | |
| nogRNA-G1397spilt-KKH- | C | 0.00% | 0.01% | 0.02% | 0.00% | 0.00% | 0.00% | 0.00% | 99.76% |
| Cas9-G1397-N and | | | | | | | | | |
| dSpCas9-G1397-C | | | | | | | | | |
| nogRNA-G1397spilt-KKH- | G | 99.83% | 0.00% | 0.00% | 99.85% | 0.00% | 99.75% | 99.75% | 0.00% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.14% | 0.14% | 0.14% | 0.14% | 0.14% | 0.23% | 0.23% | 0.23% |
| Cas9-G1397-N and | | | | | | | | | |
| dSpCas9-G1397-C | | | | | | | | | |
| nogRNA-G1397spilt-KKH- | A | 0.01% | 0.01% | 0.00% | 0.01% | 99.97% | 0.01% | 0.00% | 0.00% |
| Cas9-G1397-C and | | | | | | | | | |
| dSpCas9-G1397-N | | | | | | | | | |
| nogRNA-G1397spilt-KKH- | T | 0.01% | 99.97% | 99.98% | 0.00% | 0.01% | 0.01% | 0.00% | 0.01% |
| Cas9-G1397-C and | | | | | | | | | |
| dSpCas9-G1397-N | | | | | | | | | |
| nogRNA-G1397spilt-KKH- | C | 0.00% | 0.01% | 0.02% | 0.00% | 0.01% | 0.00% | 0.01% | 99.98% |
| Cas9-G1397-C and | | | | | | | | | |
| dSpCas9-G1397-N | | | | | | | | | |
| nogRNA-G1397spilt-KKH- | G | 99.98% | 0.01% | 0.00% | 99.98% | 0.01% | 99.97% | 99.98% | 0.01% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.00% | 0.00% | 0.00% | 0.01% | 0.01% | 0.01% | 0.00% | 0.00% |
| Cas9-G1397-C and | | | | | | | | | |
| dSpCas9-G1397-N | | | | | | | | | |
| nogRNA-N1357spilt-KKH- | A | 0.01% | 0.01% | 0.00% | 0.02% | 99.97% | 0.01% | 0.01% | 0.00% |
| Cas9-N1357-N and | | | | | | | | | |
| dSpCas9-N1357-C | | | | | | | | | |
| nogRNA-N1357spilt-KKH- | T | 0.01% | 99.97% | 99.99% | 0.01% | 0.01% | 0.02% | 0.00% | 0.01% |
| Cas9-N1357-N and | | | | | | | | | |
| dSpCas9-N1357-C | | | | | | | | | |
| nogRNA-N1357spilt-KKH- | C | 0.00% | 0.01% | 0.01% | 0.00% | 0.00% | 0.00% | 0.00% | 99.98% |
| Cas9-N1357-N and | | | | | | | | | |
| dSpCas9-N1357-C | | | | | | | | | |
| nogRNA-N1357spilt-KKH- | G | 99.98% | 0.01% | 0.00% | 99.97% | 0.01% | 99.97% | 99.99% | 0.00% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.00% | 0.00% | 0.00% | 0.01% | 0.01% | 0.01% | 0.00% | 0.00% |
| Cas9-N1357-N and | | | | | | | | | |
| dSpCas9-N1357-C | | | | | | | | | |
| nogRNA-N1357spilt-KKH- | A | 0.01% | 0.01% | 0.00% | 0.02% | 99.95% | 0.01% | 0.00% | 0.00% |
| Cas9-N1357-C and | | | | | | | | | |
| dSpCas9-N1357-N | | | | | | | | | |
| nogRNA-N1357spilt-KKH- | T | 0.01% | 99.98% | 99.98% | 0.00% | 0.00% | 0.02% | 0.01% | 0.01% |
| Cas9-N1357-C and | | | | | | | | | |
| dSpCas9-N1357-N | | | | | | | | | |
| nogRNA-N1357spilt-KKH- | C | 0.00% | 0.01% | 0.02% | 0.00% | 0.02% | 0.00% | 0.00% | 99.98% |
| Cas9-N1357-C and | | | | | | | | | |
| dSpCas9-N1357-N | | | | | | | | | |
| nogRNA-N1357spilt-KKH- | G | 99.99% | 0.01% | 0.00% | 99.97% | 0.02% | 99.96% | 99.99% | 0.01% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.00% | 0.00% | 0.00% | 0.01% | 0.01% | 0.01% | 0.00% | 0.00% |
| Cas9-N1357-C and | | | | | | | | | |
| dSpCas9-N1357-N | | | | | | | | | |
| nogRNA-N1387spilt-KKH- | A | 0.00% | 0.00% | 0.01% | 0.02% | 99.95% | 0.01% | 0.02% | 0.00% |
| Cas9-N1387-N and | | | | | | | | | |
| dSpCas9-N1387-C | | | | | | | | | |
| nogRNA-N1387spilt-KKH- | T | 0.01% | 99.96% | 99.96% | 0.00% | 0.01% | 0.01% | 0.01% | 0.02% |
| Cas9-N1387-N and | | | | | | | | | |
| dSpCas9-N1387-C | | | | | | | | | |
| nogRNA-N1387spilt-KKH- | C | 0.00% | 0.02% | 0.01% | 0.00% | 0.00% | 0.01% | 0.00% | 99.97% |
| Cas9-N1387-N and | | | | | | | | | |
| dSpCas9-N1387-C | | | | | | | | | |
| nogRNA-N1387spilt-KKH- | G | 99.97% | 0.00% | 0.01% | 99.96% | 0.02% | 99.96% | 99.94% | 0.00% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.01% | 0.01% | 0.01% | 0.02% | 0.01% | 0.01% | 0.02% | 0.01% |
| Cas9-N1387-N and | | | | | | | | | |
| dSpCas9-N1387-C | | | | | | | | | |
| nogRNA-N1387spilt-KKH- | A | 0.01% | 0.01% | 0.00% | 0.01% | 99.96% | 0.00% | 0.01% | 0.01% |
| Cas9-N1387-C and | | | | | | | | | |
| dSpCas9-N1387-N | | | | | | | | | |
| nogRNA-N1387spilt-KKH- | T | 0.00% | 99.96% | 99.98% | 0.00% | 0.01% | 0.01% | 0.00% | 0.00% |
| Cas9-N1387-C and | | | | | | | | | |
| dSpCas9-N1387-N | | | | | | | | | |
| nogRNA-N1387spilt-KKH- | C | 0.00% | 0.01% | 0.02% | 0.00% | 0.01% | 0.00% | 0.00% | 99.96% |
| Cas9-N1387-C and | | | | | | | | | |
| dSpCas9-N1387-N | | | | | | | | | |
| nogRNA-N1387spilt-KKH- | G | 99.99% | 0.01% | 0.00% | 99.99% | 0.01% | 99.99% | 99.98% | 0.02% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |

| Batch | Nucleotide | C | C | C | A | G | T | G |
|---|---|---|---|---|---|---|---|---|
| nogRNA-A1343spilt-KKH- | A | 0.00% | 0.00% | 0.00% | 99.97% | 0.01% | 0.00% | 0.00% |
| Cas9-A1343-N and | | | | | | | | |
| dSpCas9-A1343-C | | | | | | | | |
| nogRNA-A1343spilt-KKH- | T | 0.03% | 0.01% | 0.01% | 0.00% | 0.00% | 99.98% | 0.00% |
| Cas9-A1343-N and | | | | | | | | |
| dSpCas9-A1343-C | | | | | | | | |
| nogRNA-A1343spilt-KKH- | C | 99.97% | 99.98% | 99.96% | 0.00% | 0.00% | 0.01% | 0.00% |
| Cas9-A1343-N and | | | | | | | | |
| dSpCas9-A1343-C | | | | | | | | |
| nogRNA-A1343spilt-KKH- | G | 0.00% | 0.00% | 0.00% | 0.02% | 99.98% | 0.01% | 99.99% |
| Cas9-A1343-N and | | | | | | | | |
| dSpCas9-A1343-C | | | | | | | | |
| nogRNA-A1343spilt-KKH- | N | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | — | 0.00% | 0.00% | 0.03% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | A | 0.00% | 0.01% | 0.00% | 99.95% | 0.00% | 0.00% | 0.02% |
| Cas9-A1343-C and | | | | | | | | |
| dSpCas9-A1343-N | | | | | | | | |
| nogRNA-A1343spilt-KKH- | T | 0.01% | 0.01% | 0.00% | 0.00% | 0.00% | 99.95% | 0.01% |
| Cas9-A1343-C and | | | | | | | | |
| dSpCas9-A1343-N | | | | | | | | |
| nogRNA-A1343spilt-KKH- | C | 99.99% | 99.97% | 99.99% | 0.04% | 0.00% | 0.02% | 0.00% |
| Cas9-A1343-C and | | | | | | | | |
| dSpCas9-A1343-N | | | | | | | | |
| nogRNA-A1343spilt-KKH- | G | 0.00% | 0.00% | 0.00% | 0.02% | 100.00% | 0.02% | 99.97% |
| Cas9-A1343-C and | | | | | | | | |
| dSpCas9-A1343-N | | | | | | | | |
| nogRNA-A1343spilt-KKH- | N | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-A1343spilt-KKH- | — | 0.00% | 0.00% | 0.01% | 0.00% | 0.00% | 0.00% | 0.00% |
| Cas9-A1343-C and | | | | | | | | |
| dSpCas9-A1343-N | | | | | | | | |
| nogRNA-G1322spilt-KKH- | A | 0.00% | 0.00% | 0.01% | 99.94% | 0.00% | 0.01% | 0.01% |
| Cas9-G1322-N and | | | | | | | | |
| dSpCas9-G1322-C | | | | | | | | |
| nogRNA-G1322spilt-KKH- | T | 0.00% | 0.02% | 0.01% | 0.01% | 0.00% | 99.96% | 0.01% |
| Cas9-G1322-N and | | | | | | | | |
| dSpCas9-G1322-C | | | | | | | | |
| nogRNA-G1322spilt-KKH- | C | 99.99% | 99.98% | 99.93% | 0.01% | 0.00% | 0.01% | 0.01% |
| Cas9-G1322-N and | | | | | | | | |
| dSpCas9-G1322-C | | | | | | | | |
| nogRNA-G1322spilt-KKH- | G | 0.00% | 0.00% | 0.00% | 0.05% | 10.000% | 0.01% | 99.97% |
| Cas9-G1322-N and | | | | | | | | |
| dSpCas9-G1322-C | | | | | | | | |
| nogRNA-G1322spilt-KKH- | N | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | — | 0.00% | 0.00% | 0.05% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | A | 0.00% | 0.01% | 0.01% | 99.96% | 0.00% | 0.01% | 0.02% |
| Cas9-G1322-C and | | | | | | | | |
| dSpCas9-G1322-N | | | | | | | | |
| nogRNA-G1322spilt-KKH- | T | 0.00% | 0.01% | 0.00% | 0.00% | 0.00% | 99.95% | 0.01% |
| Cas9-G1322-C and | | | | | | | | |
| dSpCas9-G1322-N | | | | | | | | |
| nogRNA-G1322spilt-KKH- | C | 10.000% | 99.97% | 99.95% | 0.01% | 0.00% | 0.03% | 0.00% |
| Cas9-G1322-C and | | | | | | | | |
| dSpCas9-G1322-N | | | | | | | | |
| nogRNA-G1322spilt-KKH- | G | 0.00% | 0.00% | 0.00% | 0.02% | 99.99% | 0.00% | 99.97% |
| Cas9-G1322-C and | | | | | | | | |
| dSpCas9-G1322-N | | | | | | | | |
| nogRNA-G1322spilt-KKH- | N | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1322spilt-KKH- | — | 0.00% | 0.01% | 0.03% | 0.00% | 0.00% | 0.00% | 0.00% |
| Cas9-G1322-C and | | | | | | | | |
| dSpCas9-G1322-N | | | | | | | | |
| nogRNA-G1333spilt-KKH- | A | 0.00% | 0.01% | 0.01% | 99.94% | 0.00% | 0.00% | 0.02% |
| Cas9-G1333-N and | | | | | | | | |
| dSpCas9-G1333-C | | | | | | | | |
| nogRNA-G1333spilt-KKH- | T | 0.00% | 0.01% | 0.01% | 0.01% | 0.00% | 99.95% | 0.00% |
| Cas9-G1333-N and | | | | | | | | |
| dSpCas9-G1333-C | | | | | | | | |
| nogRNA-G1333spilt-KKH- | C | 99.99% | 99.96% | 99.94% | 0.01% | 0.00% | 0.02% | 0.00% |
| Cas9-G1333-N and | | | | | | | | |
| dSpCas9-G1333-C | | | | | | | | |
| nogRNA-G1333spilt-KKH- | G | 0.00% | 0.00% | 0.01% | 0.04% | 100.00% | 0.02% | 99.97% |
| Cas9-G1333-N and | | | | | | | | |
| dSpCas9-G1333-C | | | | | | | | |
| nogRNA-G1333spilt-KKH- | N | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | — | 0.00% | 0.01% | 0.03% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | A | 0.00% | 0.02% | 0.00% | 99.97% | 0.01% | 0.00% | 0.01% |
| Cas9-G1333-C and | | | | | | | | |
| dSpCas9-G1333-N | | | | | | | | |
| nogRNA-G1333spilt-KKH- | T | 0.00% | 0.02% | 0.02% | 0.00% | 0.01% | 99.97% | 0.00% |
| Cas9-G1333-C and | | | | | | | | |
| dSpCas9-G1333-N | | | | | | | | |
| nogRNA-G1333spilt-KKH- | C | 99.99% | 99.96% | 99.94% | 0.01% | 0.00% | 0.03% | 0.00% |
| Cas9-G1333-C and | | | | | | | | |
| dSpCas9-G1333-N | | | | | | | | |
| nogRNA-G1333spilt-KKH- | G | 0.00% | 0.00% | 0.01% | 0.02% | 99.97% | 0.00% | 99.98% |
| Cas9-G1333-C and | | | | | | | | |
| dSpCas9-G1333-N | | | | | | | | |
| nogRNA-G1333spilt-KKH- | N | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1333spilt-KKH- | — | 0.00% | 0.00% | 0.04% | 0.00% | 0.00% | 0.00% | 0.00% |
| Cas9-G1333-C and | | | | | | | | |
| dSpCas9-G1333-N | | | | | | | | |
| nogRNA-G1371spilt-KKH- | A | 0.00% | 0.00% | 0.00% | 99.96% | 0.00% | 0.01% | 0.03% |
| Cas9-G1371-N and | | | | | | | | |
| dSpCas9-G1371-C | | | | | | | | |
| nogRNA-G1371spilt-KKH- | T | 0.00% | 0.00% | 0.01% | 0.01% | 0.00% | 99.98% | 0.01% |
| Cas9-G1371-N and | | | | | | | | |
| dSpCas9-G1371-C | | | | | | | | |
| nogRNA-G1371spilt-KKH- | C | 100.00% | 99.98% | 99.96% | 0.00% | 0.00% | 0.01% | 0.00% |
| Cas9-G1371-N and | | | | | | | | |
| dSpCas9-G1371-C | | | | | | | | |
| nogRNA-G1371spilt-KKH- | G | 0.00% | 0.01% | 0.00% | 0.03% | 99.99% | 0.01% | 99.96% |
| Cas9-G1371-N and | | | | | | | | |
| dSpCas9-G1371-C | | | | | | | | |
| nogRNA-G1371spilt-KKH- | N | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | — | 0.00% | 0.00% | 0.02% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | A | 0.01% | 0.01% | 0.01% | 99.96% | 0.01% | 0.01% | 0.01% |
| Cas9-G1371-C and | | | | | | | | |
| dSpCas9-G1371-N | | | | | | | | |
| nogRNA-G1371spilt-KKH- | T | 0.01% | 0.01% | 0.02% | 0.01% | 0.00% | 99.95% | 0.01% |
| Cas9-G1371-C and | | | | | | | | |
| dSpCas9-G1371-N | | | | | | | | |
| nogRNA-G1371spilt-KKH- | C | 99.96% | 99.97% | 99.95% | 0.00% | 0.00% | 0.01% | 0.00% |
| Cas9-G1371-C and | | | | | | | | |
| dSpCas9-G1371-N | | | | | | | | |
| nogRNA-G1371spilt-KKH- | G | 0.01% | 0.00% | 0.00% | 0.02% | 99.99% | 0.02% | 99.97% |
| Cas9-G1371-C and | | | | | | | | |
| dSpCas9-G1371-N | | | | | | | | |
| nogRNA-G1371spilt-KKH- | N | 0.00% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1371spilt-KKH- | — | 0.01% | 0.01% | 0.02% | 0.01% | 0.01% | 0.01% | 0.01% |
| Cas9-G1371-C and | | | | | | | | |
| dSpCas9-G1371-N | | | | | | | | |
| nogRNA-G1397spilt-KKH- | A | 0.00% | 0.01% | 0.01% | 99.74% | 0.01% | 0.01% | 0.01% |
| Cas9-G1397-N and | | | | | | | | |
| dSpCas9-G1397-C | | | | | | | | |
| nogRNA-G1397spilt-KKH- | T | 0.01% | 0.00% | 0.00% | 0.00% | 0.01% | 99.74% | 0.03% |
| Cas9-G1397-N and | | | | | | | | |
| dSpCas9-G1397-C | | | | | | | | |
| nogRNA-G1397spilt-KKH- | C | 99.75% | 99.76% | 99.72% | 0.00% | 0.00% | 0.01% | 0.00% |
| Cas9-G1397-N and | | | | | | | | |
| dSpCas9-G1397-C | | | | | | | | |
| nogRNA-G1397spilt-KKH- | G | 0.00% | 0.00% | 0.00% | 0.02% | 99.75% | 0.01% | 99.73% |
| Cas9-G1397-N and | | | | | | | | |
| dSpCas9-G1397-C | | | | | | | | |
| nogRNA-G1397spilt-KKH- | N | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | — | 0.23% | 0.23% | 0.26% | 0.23% | 0.23% | 0.23% | 0.23% |
| Cas9-G1397-N and | | | | | | | | |
| dSpCas9-G1397-C | | | | | | | | |
| nogRNA-G1397spilt-KKH- | A | 0.00% | 0.00% | 0.01% | 99.96% | 0.00% | 0.00% | 0.01% |
| Cas9-G1397-C and | | | | | | | | |
| dSpCas9-G1397-N | | | | | | | | |
| nogRNA-G1397spilt-KKH- | T | 0.00% | 0.02% | 0.01% | 0.00% | 0.00% | 99.94% | 0.01% |
| Cas9-G1397-C and | | | | | | | | |
| dSpCas9-G1397-N | | | | | | | | |
| nogRNA-G1397spilt-KKH- | C | 100.00% | 99.97% | 99.95% | 0.00% | 0.00% | 0.02% | 0.00% |
| Cas9-G1397-C and | | | | | | | | |
| dSpCas9-G1397-N | | | | | | | | |
| nogRNA-G1397spilt-KKH- | G | 0.00% | 0.00% | 0.00% | 0.03% | 99.98% | 0.02% | 99.96% |
| Cas9-G1397-C and | | | | | | | | |
| dSpCas9-G1397-N | | | | | | | | |
| nogRNA-G1397spilt-KKH- | N | 0.00% | 0.00% | 0.00% | 0.00% | 0.00% | 0.00% | 0.00% |
| Cas9-G1397-C and | | | | | | | | |
| dSpCas9-G1397-N | | | | | | | | |
| nogRNA-G1397spilt-KKH- | — | 0.00% | 0.00% | 0.03% | 0.01% | 0.01% | 0.01% | 0.01% |
| Cas9-G1397-C and | | | | | | | | |
| dSpCas9-G1397-N | | | | | | | | |
| nogRNA-N1357spilt-KKH- | A | 0.01% | 0.00% | 0.00% | 99.98% | 0.03% | 0.01% | 0.02% |
| Cas9-N1357-N and | | | | | | | | |
| dSpCas9-N1357-C | | | | | | | | |
| nogRNA-N1357spilt-KKH- | T | 0.01% | 0.01% | 0.01% | 0.00% | 0.00% | 99.95% | 0.02% |
| Cas9-N1357-N and | | | | | | | | |
| dSpCas9-N1357-C | | | | | | | | |
| nogRNA-N1357spilt-KKH- | C | 99.98% | 99.98% | 99.95% | 0.00% | 0.00% | 0.02% | 0.01% |
| Cas9-N1357-N and | | | | | | | | |
| dSpCas9-N1357-C | | | | | | | | |
| nogRNA-N1357spilt-KKH- | G | 0.00% | 0.00% | 0.00% | 0.02% | 99.97% | 0.02% | 99.96% |
| Cas9-N1357-N and | | | | | | | | |
| dSpCas9-N1357-C | | | | | | | | |
| nogRNA-N1357spilt-KKH- | N | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | — | 0.00% | 0.00% | 0.04% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | A | 0.02% | 0.00% | 0.00% | 99.94% | 0.01% | 0.01% | 0.01% |
| Cas9-N1357-C and | | | | | | | | |
| dSpCas9-N1357-N | | | | | | | | |
| nogRNA-N1357spilt-KKH- | T | 0.00% | 0.02% | 0.02% | 0.01% | 0.00% | 99.97% | 0.01% |
| Cas9-N1357-C and | | | | | | | | |
| dSpCas9-N1357-N | | | | | | | | |
| nogRNA-N1357spilt-KKH- | C | 99.98% | 99.97% | 99.98% | 0.04% | 0.00% | 0.02% | 0.01% |
| Cas9-N1357-C and | | | | | | | | |
| dSpCas9-N1357-N | | | | | | | | |
| nogRNA-N1357spilt-KKH- | G | 0.00% | 0.01% | 0.00% | 0.01% | 99.99% | 0.00% | 99.98% |
| Cas9-N1357-C and | | | | | | | | |
| dSpCas9-N1357-N | | | | | | | | |
| nogRNA-N1357spilt-KKH- | N | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1357spilt-KKH- | — | 0.00% | 0.00% | 0.01% | 0.00% | 0.00% | 0.00% | 0.00% |
| Cas9-N1357-C and | | | | | | | | |
| dSpCas9-N1357-N | | | | | | | | |
| nogRNA-N1387spilt-KKH- | A | 0.00% | 0.00% | 0.01% | 99.96% | 0.00% | 0.01% | 0.01% |
| Cas9-N1387-N and | | | | | | | | |
| dSpCas9-N1387-C | | | | | | | | |
| nogRNA-N1387spilt-KKH- | T | 0.02% | 0.01% | 0.04% | 0.00% | 0.00% | 99.96% | 0.02% |
| Cas9-N1387-N and | | | | | | | | |
| dSpCas9-N1387-C | | | | | | | | |
| nogRNA-N1387spilt-KKH- | C | 99.97% | 99.97% | 99.89% | 0.01% | 0.00% | 0.02% | 0.00% |
| Cas9-N1387-N and | | | | | | | | |
| dSpCas9-N1387-C | | | | | | | | |
| nogRNA-N1387spilt-KKH- | G | 0.00% | 0.00% | 0.01% | 0.02% | 99.99% | 0.00% | 99.96% |
| Cas9-N1387-N and | | | | | | | | |
| dSpCas9-N1387-C | | | | | | | | |
| nogRNA-N1387spilt-KKH- | N | 0.00% | 0.00% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | — | 0.01% | 0.01% | 0.04% | 0.01% | 0.01% | 0.01% | 0.01% |
| Cas9-N1387-N and | | | | | | | | |
| dSpCas9-N1387-C | | | | | | | | |
| nogRNA-N1387spilt-KKH- | A | 0.00% | 0.00% | 0.01% | 99.95% | 0.00% | 0.01% | 0.00% |
| Cas9-N1387-C and | | | | | | | | |
| dSpCas9-N1387-N | | | | | | | | |
| nogRNA-N1387spilt-KKH- | T | 0.01% | 0.00% | 0.02% | 0.01% | 0.01% | 99.96% | 0.01% |
| Cas9-N1387-C and | | | | | | | | |
| dSpCas9-N1387-N | | | | | | | | |
| nogRNA-N1387spilt-KKH- | C | 99.98% | 99.98% | 99.92% | 0.01% | 0.00% | 0.01% | 0.00% |
| Cas9-N1387-C and | | | | | | | | |
| dSpCas9-N1387-N | | | | | | | | |
| nogRNA-N1387spilt-KKH- | G | 0.00% | 0.00% | 0.00% | 0.02% | 99.97% | 0.00% | 99.97% |
| Cas9-N1387-C and | | | | | | | | |
| dSpCas9-N1387-N | | | | | | | | |
| nogRNA-N1387spilt-KKH- | N | 0.00% | 0.00% | 0.00% | 0.00% | 0.00% | 0.00% | 0.00% |
| Cas9-N1387-C and | | | | | | | | |
| dSpCas9-N1387-N | | | | | | | | |
| nogRNA-N1387spilt-KKH- | — | 0.01% | 0.01% | 0.05% | 0.01% | 0.01% | 0.01% | 0.01% |
| Cas9-N1387-C and | | | | | | | | |
| dSpCas9-N1387-N | | | | | | | | |

| Batch | Nucleotide | G | C | T | G | C | T | C | T | G |
|---|---|---|---|---|---|---|---|---|---|---|
| nogRNA-A1343spilt-KKH- | A | 0.01% | 0.01% | 0.00% | 0.01% | 0.01% | 0.00% | 0.00% | 0.00% | 0.02% |
| Cas9-A1343-N and | | | | | | | | | | |
| dSpCas9-A1343-C | | | | | | | | | | |
| nogRNA-A1343spilt-KKH- | T | 0.00% | 0.01% | 99.98% | 0.00% | 0.00% | 99.98% | 0.00% | 99.98% | 0.00% |
| Cas9-A1343-N and | | | | | | | | | | |
| dSpCas9-A1343-C | | | | | | | | | | |
| nogRNA-A1343spilt-KKH- | C | 0.01% | 99.98% | 0.02% | 0.00% | 99.99% | 0.01% | 100.00% | 0.02% | 0.01% |
| Cas9-A1343-N and | | | | | | | | | | |
| dSpCas9-A1343-C | | | | | | | | | | |
| nogRNA-A1343spilt-KKH- | G | 99.98% | 0.00% | 0.00% | 99.99% | 0.00% | 0.01% | 0.00% | 0.00% | 99.97% |
| Cas9-A1343-N and | | | | | | | | | | |
| dSpCas9-A1343-C | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | A | 0.01% | 0.00% | 0.01% | 0.00% | 0.00% | 0.00% | 0.00% | 0.00% | 0.01% |
| Cas9-A1343-C and | | | | | | | | | | |
| dSpCas9-A1343-N | | | | | | | | | | |
| nogRNA-A1343spilt-KKH- | T | 0.00% | 0.01% | 99.96% | 0.01% | 0.00% | 99.99% | 0.00% | 99.98% | 0.01% |
| Cas9-A1343-C and | | | | | | | | | | |
| dSpCas9-A1343-N | | | | | | | | | | |
| nogRNA-A1343spilt-KKH- | C | 0.00% | 99.99% | 0.03% | 0.00% | 99.99% | 0.00% | 100.00% | 0.02% | 0.00% |
| Cas9-A1343-C and | | | | | | | | | | |
| dSpCas9-A1343-N | | | | | | | | | | |
| nogRNA-A1343spilt-KKH- | G | 99.99% | 0.00% | 0.00% | 99.98% | 0.01% | 0.01% | 0.00% | 0.00% | 99.98% |
| Cas9-A1343-C and | | | | | | | | | | |
| dSpCas9-A1343-N | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-G1322spilt-KKH- | A | 0.01% | 0.00% | 0.00% | 0.00% | 0.00% | 0.00% | 0.00% | 0.00% | 0.01% |
| Cas9-G1322-N and | | | | | | | | | | |
| dSpCas9-G1322-C | | | | | | | | | | |
| nogRNA-G1322spilt-KKH- | T | 0.01% | 0.01% | 99.98% | 0.02% | 0.00% | 99.98% | 0.01% | 99.97% | 0.02% |
| Cas9-G1322-N and | | | | | | | | | | |
| dSpCas9-G1322-C | | | | | | | | | | |
| nogRNA-G1322spilt-KKH- | C | 0.00% | 99.99% | 0.01% | 0.00% | 99.99% | 0.00% | 99.99% | 0.02% | 0.00% |
| Cas9-G1322-N and | | | | | | | | | | |
| dSpCas9-G1322-C | | | | | | | | | | |
| nogRNA-G1322spilt-KKH- | G | 99.98% | 0.00% | 0.00% | 99.97% | 0.00% | 0.01% | 0.00% | 0.01% | 99.96% |
| Cas9-G1322-N and | | | | | | | | | | |
| dSpCas9-G1322-C | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | A | 0.00% | 0.00% | 0.01% | 0.03% | 0.00% | 0.00% | 0.01% | 0.01% | 0.01% |
| Cas9-G1322-C and | | | | | | | | | | |
| dSpCas9-G1322-N | | | | | | | | | | |
| nogRNA-G1322spilt-KKH- | T | 0.00% | 0.01% | 99.98% | 0.01% | 0.00% | 99.97% | 0.03% | 99.97% | 0.01% |
| Cas9-G1322-C and | | | | | | | | | | |
| dSpCas9-G1322-N | | | | | | | | | | |
| nogRNA-G1322spilt-KKH- | C | 0.00% | 99.99% | 0.01% | 0.00% | 99.99% | 0.02% | 99.97% | 0.02% | 0.01% |
| Cas9-G1322-C and | | | | | | | | | | |
| dSpCas9-G1322-N | | | | | | | | | | |
| nogRNA-G1322spilt-KKH- | G | 99.99% | 0.00% | 0.00% | 99.96% | 0.00% | 0.01% | 0.00% | 0.00% | 99.97% |
| Cas9-G1322-C and | | | | | | | | | | |
| dSpCas9-G1322-N | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1333spilt-KKH- | A | 0.01% | 0.00% | 0.01% | 0.01% | 0.00% | 0.00% | 0.01% | 0.01% | 0.02% |
| Cas9-G1333-N and | | | | | | | | | | |
| dSpCas9-G1333-C | | | | | | | | | | |
| nogRNA-G1333spilt-KKH- | T | 0.01% | 0.00% | 99.98% | 0.02% | 0.01% | 99.97% | 0.01% | 99.98% | 0.00% |
| Cas9-G1333-N and | | | | | | | | | | |
| dSpCas9-G1333-C | | | | | | | | | | |
| nogRNA-G1333spilt-KKH- | C | 0.00% | 99.99% | 0.01% | 0.00% | 99.99% | 0.01% | 99.97% | 0.00% | 0.00% |
| Cas9-G1333-N and | | | | | | | | | | |
| dSpCas9-G1333-C | | | | | | | | | | |
| nogRNA-G1333spilt-KKH- | G | 99.98% | 0.00% | 0.00% | 99.97% | 0.00% | 0.01% | 0.00% | 0.00% | 99.97% |
| Cas9-G1333-N and | | | | | | | | | | |
| dSpCas9-G1333-C | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | A | 0.01% | 0.00% | 0.00% | 0.01% | 0.00% | 0.00% | 0.00% | 0.00% | 0.01% |
| Cas9-G1333-C and | | | | | | | | | | |
| dSpCas9-G1333-N | | | | | | | | | | |
| nogRNA-G1333spilt-KKH- | T | 0.01% | 0.00% | 99.97% | 0.02% | 0.00% | 99.98% | 0.00% | 99.99% | 0.00% |
| Cas9-G1333-C and | | | | | | | | | | |
| dSpCas9-G1333-N | | | | | | | | | | |
| nogRNA-G1333spilt-KKH- | C | 0.00% | 100.00% | 0.03% | 0.00% | 99.99% | 0.01% | 100.00% | 0.01% | 0.01% |
| Cas9-G1333-C and | | | | | | | | | | |
| dSpCas9-G1333-N | | | | | | | | | | |
| nogRNA-G1333spilt-KKH- | G | 99.97% | 0.00% | 0.00% | 99.97% | 0.00% | 0.00% | 0.00% | 0.00% | 99.97% |
| Cas9-G1333-C and | | | | | | | | | | |
| dSpCas9-G1333-N | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1333spilt-KKH- | — | 0.01% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1371spilt-KKH- | A | 0.01% | 0.00% | 0.01% | 0.01% | 0.00% | 0.01% | 0.00% | 0.01% | 0.00% |
| Cas9-G1371-N and | | | | | | | | | | |
| dSpCas9-G1371-C | | | | | | | | | | |
| nogRNA-G1371spilt-KKH- | T | 0.00% | 0.00% | 99.97% | 0.00% | 0.01% | 99.97% | 0.03% | 99.97% | 0.02% |
| Cas9-G1371-N and | | | | | | | | | | |
| dSpCas9-G1371-C | | | | | | | | | | |
| nogRNA-G1371spilt-KKH- | C | 0.00% | 99.99% | 0.02% | 0.00% | 99.99% | 0.02% | 99.97% | 0.02% | 0.00% |
| Cas9-G1371-N and | | | | | | | | | | |
| dSpCas9-G1371-C | | | | | | | | | | |
| nogRNA-G1371spilt-KKH- | G | 99.98% | 0.00% | 0.00% | 99.98% | 0.00% | 0.00% | 0.00% | 0.00% | 99.97% |
| Cas9-G1371-N and | | | | | | | | | | |
| dSpCas9-G1371-C | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | — | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | A | 0.00% | 0.00% | 0.00% | 0.01% | 0.00% | 0.00% | 0.00% | 0.01% | 0.01% |
| Cas9-G1371-C and | | | | | | | | | | |
| dSpCas9-G1371-N | | | | | | | | | | |
| nogRNA-G1371spilt-KKH- | T | 0.01% | 0.00% | 99.99% | 0.00% | 0.00% | 99.96% | 0.01% | 99.96% | 0.01% |
| Cas9-G1371-C and | | | | | | | | | | |
| dSpCas9-G1371-N | | | | | | | | | | |
| nogRNA-G1371spilt-KKH- | C | 0.00% | 99.99% | 0.01% | 0.00% | 99.99% | 0.02% | 99.97% | 0.01% | 0.00% |
| Cas9-G1371-C and | | | | | | | | | | |
| dSpCas9-G1371-N | | | | | | | | | | |
| nogRNA-G1371spilt-KKH- | G | 99.97% | 0.00% | 0.00% | 99.98% | 0.00% | 0.01% | 0.00% | 0.00% | 99.97% |
| Cas9-G1371-C and | | | | | | | | | | |
| dSpCas9-G1371-N | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1371spilt-KKH- | — | 0.01% | 0.01% | 0.01% | 0.01% | 0.01% | 0.01% | 0.01% | 0.01% | 0.01% |
| Cas9-G1371-C and | | | | | | | | | | |
| dSpCas9-G1371-N | | | | | | | | | | |
| nogRNA-G1397spilt-KKH- | A | 0.00% | 0.01% | 0.01% | 0.03% | 0.00% | 0.00% | 0.00% | 0.00% | 0.01% |
| Cas9-G1397-N and | | | | | | | | | | |
| dSpCas9-G1397-C | | | | | | | | | | |
| nogRNA-G1397spilt-KKH- | T | 0.00% | 0.00% | 99.70% | 0.00% | 0.00% | 99.70% | 0.01% | 99.70% | 0.01% |
| Cas9-G1397-N and | | | | | | | | | | |
| dSpCas9-G1397-C | | | | | | | | | | |
| nogRNA-G1397spilt-KKH- | C | 0.00% | 99.71% | 0.01% | 0.00% | 99.72% | 0.01% | 99.71% | 0.02% | 0.00% |
| Cas9-G1397-N and | | | | | | | | | | |
| dSpCas9-G1397-C | | | | | | | | | | |
| nogRNA-G1397spilt-KKH- | G | 99.76% | 0.00% | 0.00% | 99.69% | 0.00% | 0.01% | 0.00% | 0.00% | 99.70% |
| Cas9-G1397-N and | | | | | | | | | | |
| dSpCas9-G1397-C | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | — | 0.23% | 0.27% | 0.27% | 0.28% | 0.28% | 0.28% | 0.28% | 0.28% | 0.28% |
| Cas9-G1397-N and | | | | | | | | | | |
| dSpCas9-G1397-C | | | | | | | | | | |
| nogRNA-G1397spilt-KKH- | A | 0.01% | 0.00% | 0.00% | 0.02% | 0.00% | 0.00% | 0.01% | 0.01% | 0.01% |
| Cas9-G1397-C and | | | | | | | | | | |
| dSpCas9-G1397-N | | | | | | | | | | |
| nogRNA-G1397spilt-KKH- | T | 0.01% | 0.02% | 99.96% | 0.01% | 0.00% | 99.97% | 0.01% | 99.96% | 0.00% |
| Cas9-G1397-C and | | | | | | | | | | |
| dSpCas9-G1397-N | | | | | | | | | | |
| nogRNA-G1397spilt-KKH- | C | 0.00% | 99.96% | 0.01% | 0.00% | 99.98% | 0.00% | 99.96% | 0.00% | 0.00% |
| Cas9-G1397-C and | | | | | | | | | | |
| dSpCas9-G1397-N | | | | | | | | | | |
| nogRNA-G1397spilt-KKH- | G | 99.97% | 0.00% | 0.00% | 99.95% | 0.00% | 0.01% | 0.00% | 0.01% | 99.96% |
| Cas9-G1397-C and | | | | | | | | | | |
| dSpCas9-G1397-N | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-G1397spilt-KKH- | — | 0.01% | 0.02% | 0.02% | 0.02% | 0.02% | 0.02% | 0.02% | 0.02% | 0.02% |
| Cas9-G1397-C and | | | | | | | | | | |
| dSpCas9-G1397-N | | | | | | | | | | |
| nogRNA-N1357spilt-KKH- | A | 0.00% | 0.00% | 0.00% | 0.01% | 0.00% | 0.00% | 0.00% | 0.00% | 0.01% |
| Cas9-N1357-N and | | | | | | | | | | |
| dSpCas9-N1357-C | | | | | | | | | | |
| nogRNA-N1357spilt-KKH- | T | 0.00% | 0.01% | 99.97% | 0.00% | 0.01% | 99.98% | 0.00% | 99.99% | 0.01% |
| Cas9-N1357-N and | | | | | | | | | | |
| dSpCas9-N1357-C | | | | | | | | | | |
| nogRNA-N1357spilt-KKH- | C | 0.00% | 99.98% | 0.03% | 0.00% | 99.99% | 0.01% | 100.00% | 0.00% | 0.00% |
| Cas9-N1357-N and | | | | | | | | | | |
| dSpCas9-N1357-C | | | | | | | | | | |
| nogRNA-N1357spilt-KKH- | G | 99.99% | 0.01% | 0.00% | 99.99% | 0.00% | 0.01% | 0.00% | 0.00% | 99.98% |
| Cas9-N1357-N and | | | | | | | | | | |
| dSpCas9-N1357-C | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | A | 0.02% | 0.00% | 0.00% | 0.02% | 0.00% | 0.00% | 0.01% | 0.01% | 0.01% |
| Cas9-N1357-C and | | | | | | | | | | |
| dSpCas9-N1357-N | | | | | | | | | | |
| nogRNA-N1357spilt-KKH- | T | 0.01% | 0.01% | 99.99% | 0.01% | 0.03% | 99.97% | 0.00% | 99.99% | 0.01% |
| Cas9-N1357-C and | | | | | | | | | | |
| dSpCas9-N1357-N | | | | | | | | | | |
| nogRNA-N1357spilt-KKH- | C | 0.00% | 99.99% | 0.01% | 0.00% | 99.97% | 0.02% | 99.99% | 0.01% | 0.00% |
| Cas9-N1357-C and | | | | | | | | | | |
| dSpCas9-N1357-N | | | | | | | | | | |
| nogRNA-N1357spilt-KKH- | G | 99.97% | 0.00% | 0.01% | 99.98% | 0.00% | 0.02% | 0.00% | 0.00% | 99.98% |
| Cas9-N1357-C and | | | | | | | | | | |
| dSpCas9-N1357-N | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1387spilt-KKH- | A | 0.00% | 0.01% | 0.00% | 0.01% | 0.01% | 0.02% | 0.00% | 0.01% | 0.02% |
| Cas9-N1387-N and | | | | | | | | | | |
| dSpCas9-N1387-C | | | | | | | | | | |
| nogRNA-N1387spilt-KKH- | T | 0.00% | 0.01% | 99.97% | 0.01% | 0.00% | 99.96% | 0.00% | 99.95% | 0.01% |
| Cas9-N1387-N and | | | | | | | | | | |
| dSpCas9-N1387-C | | | | | | | | | | |
| nogRNA-N1387spilt-KKH- | C | 0.00% | 99.97% | 0.01% | 0.00% | 99.98% | 0.00% | 99.98% | 0.02% | 0.01% |
| Cas9-N1387-N and | | | | | | | | | | |
| dSpCas9-N1387-C | | | | | | | | | | |
| nogRNA-N1387spilt-KKH- | G | 99.98% | 0.00% | 0.00% | 99.97% | 0.00% | 0.00% | 0.00% | 0.00% | 99.95% |
| Cas9-N1387-N and | | | | | | | | | | |
| dSpCas9-N1387-C | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | — | 0.01% | 0.01% | 0.01% | 0.01% | 0.01% | 0.01% | 0.01% | 0.01% | 0.01% |
| Cas9-N1387-N and | | | | | | | | | | |
| dSpCas9-N1387-C | | | | | | | | | | |
| nogRNA-N1387spilt-KKH- | A | 0.00% | 0.00% | 0.00% | 0.03% | 0.00% | 0.01% | 0.01% | 0.00% | 0.00% |
| Cas9-N1387-C and | | | | | | | | | | |
| dSpCas9-N1387-N | | | | | | | | | | |
| nogRNA-N1387spilt-KKH- | T | 0.00% | 0.00% | 99.94% | 0.00% | 0.00% | 99.97% | 0.01% | 99.98% | 0.01% |
| Cas9-N1387-C and | | | | | | | | | | |
| dSpCas9-N1387-N | | | | | | | | | | |
| nogRNA-N1387spilt-KKH- | C | 0.00% | 99.99% | 0.04% | 0.00% | 99.99% | 0.01% | 99.97% | 0.01% | 0.00% |
| Cas9-N1387-C and | | | | | | | | | | |
| dSpCas9-N1387-N | | | | | | | | | | |
| nogRNA-N1387spilt-KKH- | G | 99.98% | 0.00% | 0.00% | 99.96% | 0.00% | 0.00% | 0.00% | 0.00% | 99.97% |
| Cas9-N1387-C and | | | | | | | | | | |
| dSpCas9-N1387-N | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |
| nogRNA-N1387spilt-KKH- | — | 0.01% | 0.01% | 0.01% | 0.01% | 0.01% | 0.01% | 0.01% | 0.01% | 0.01% |
| Cas9-N1387-C and | | | | | | | | | | |
| dSpCas9-N1387-N | | | | | | | | | | |

| Batch | Nucleotide | G | G | G | G | C | C | T | C |
|---|---|---|---|---|---|---|---|---|---|
| nogRNA-A1343spilt-KKH- | A | 0.01% | 0.00% | 0.02% | 0.00% | 0.00% | 0.02% | 0.00% | 0.00% |
| Cas9-A1343-N and | | | | | | | | | |
| dSpCas9-A1343-C | | | | | | | | | |
| nogRNA-A1343spilt-KKH- | T | 0.01% | 0.02% | 0.01% | 0.01% | 0.01% | 0.02% | 99.98% | 0.02% |
| Cas9-A1343-N and | | | | | | | | | |
| dSpCas9-A1343-C | | | | | | | | | |
| nogRNA-A1343spilt-KKH- | C | 0.00% | 0.00% | 0.01% | 0.00% | 99.99% | 99.95% | 0.02% | 99.98% |
| Cas9-A1343-N and | | | | | | | | | |
| dSpCas9-A1343-C | | | | | | | | | |
| nogRNA-A1343spilt-KKH- | G | 99.98% | 99.97% | 99.95% | 99.94% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.00% | 0.00% | 0.01% | 0.04% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | A | 0.00% | 0.01% | 0.02% | 0.00% | 0.00% | 0.04% | 0.00% | 0.00% |
| Cas9-A1343-C and | | | | | | | | | |
| dSpCas9-A1343-N | | | | | | | | | |
| nogRNA-A1343spilt-KKH- | T | 0.01% | 0.00% | 0.00% | 0.00% | 0.01% | 0.00% | 99.99% | 0.00% |
| Cas9-A1343-C and | | | | | | | | | |
| dSpCas9-A1343-N | | | | | | | | | |
| nogRNA-A1343spilt-KKH- | C | 0.00% | 0.00% | 0.00% | 0.00% | 99.99% | 99.95% | 0.01% | 100.00% |
| Cas9-A1343-C and | | | | | | | | | |
| dSpCas9-A1343-N | | | | | | | | | |
| nogRNA-A1343spilt-KKH- | G | 99.98% | 99.98% | 99.98% | 99.93% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.00% | 0.00% | 0.00% | 0.06% | 0.00% | 0.00% | 0.00% | 0.00% |
| Cas9-A1343-C and | | | | | | | | | |
| dSpCas9-A1343-N | | | | | | | | | |
| nogRNA-G1322spilt-KKH- | A | 0.01% | 0.00% | 0.00% | 0.02% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | T | 0.01% | 0.01% | 0.01% | 0.00% | 0.00% | 0.00% | 99.98% | 0.02% |
| Cas9-G1322-N and | | | | | | | | | |
| dSpCas9-G1322-C | | | | | | | | | |
| nogRNA-G1322spilt-KKH- | C | 0.00% | 0.00% | 0.00% | 0.00% | 10.000% | 99.99% | 0.01% | 99.98% |
| Cas9-G1322-N and | | | | | | | | | |
| dSpCas9-G1322-C | | | | | | | | | |
| nogRNA-G1322spilt-KKH- | G | 99.98% | 99.99% | 99.98% | 99.93% | 0.00% | 0.00% | 0.01% | 0.00% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.00% | 0.00% | 0.00% | 0.04% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | A | 0.02% | 0.02% | 0.02% | 0.02% | 0.00% | 0.01% | 0.00% | 0.00% |
| Cas9-G1322-C and | | | | | | | | | |
| dSpCas9-G1322-N | | | | | | | | | |
| nogRNA-G1322spilt-KKH- | T | 0.03% | 0.02% | 0.01% | 0.00% | 0.00% | 0.02% | 99.96% | 0.02% |
| Cas9-G1322-C and | | | | | | | | | |
| dSpCas9-G1322-N | | | | | | | | | |
| nogRNA-G1322spilt-KKH- | C | 0.00% | 0.00% | 0.00% | 0.00% | 99.99% | 99.97% | 0.03% | 99.98% |
| Cas9-G1322-C and | | | | | | | | | |
| dSpCas9-G1322-N | | | | | | | | | |
| nogRNA-G1322spilt-KKH- | G | 99.95% | 99.97% | 99.97% | 99.94% | 0.01% | 0.00% | 0.01% | 0.00% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.00% | 0.00% | 0.00% | 0.04% | 0.00% | 0.00% | 0.00% | 0.00% |
| Cas9-G1322-C and | | | | | | | | | |
| dSpCas9-G1322-N | | | | | | | | | |
| nogRNA-G1333spilt-KKH- | A | 0.02% | 0.01% | 0.02% | 0.01% | 0.01% | 0.02% | 0.00% | 0.00% |
| Cas9-G1333-N and | | | | | | | | | |
| dSpCas9-G1333-C | | | | | | | | | |
| nogRNA-G1333spilt-KKH- | T | 0.00% | 0.00% | 0.01% | 0.00% | 0.01% | 0.01% | 99.97% | 0.01% |
| Cas9-G1333-N and | | | | | | | | | |
| dSpCas9-G1333-C | | | | | | | | | |
| nogRNA-G1333spilt-KKH- | C | 0.00% | 0.00% | 0.00% | 0.00% | 99.97% | 99.97% | 0.03% | 99.98% |
| Cas9-G1333-N and | | | | | | | | | |
| dSpCas9-G1333-C | | | | | | | | | |
| nogRNA-G1333spilt-KKH- | G | 99.97% | 99.99% | 99.97% | 99.94% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.00% | 0.00% | 0.00% | 0.05% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | A | 0.01% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | T | 0.03% | 0.03% | 0.02% | 0.01% | 0.00% | 0.01% | 99.99% | 0.05% |
| Cas9-G1333-C and | | | | | | | | | |
| dSpCas9-G1333-N | | | | | | | | | |
| nogRNA-G1333spilt-KKH- | C | 0.00% | 0.00% | 0.00% | 0.00% | 99.99% | 99.99% | 0.00% | 99.94% |
| Cas9-G1333-C and | | | | | | | | | |
| dSpCas9-G1333-N | | | | | | | | | |
| nogRNA-G1333spilt-KKH- | G | 99.96% | 99.96% | 99.97% | 99.95% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.00% | 0.00% | 0.00% | 0.04% | 0.00% | 0.00% | 0.00% | 0.00% |
| Cas9-G1333-C and | | | | | | | | | |
| dSpCas9-G1333-N | | | | | | | | | |
| nogRNA-G1371spilt-KKH- | A | 0.01% | 0.00% | 0.00% | 0.02% | 0.00% | 0.01% | 0.01% | 0.00% |
| Cas9-G1371-N and | | | | | | | | | |
| dSpCas9-G1371-C | | | | | | | | | |
| nogRNA-G1371spilt-KKH- | T | 0.02% | 0.01% | 0.01% | 0.02% | 0.00% | 0.00% | 99.98% | 0.00% |
| Cas9-G1371-N and | | | | | | | | | |
| dSpCas9-G1371-C | | | | | | | | | |
| nogRNA-G1371spilt-KKH- | C | 0.00% | 0.00% | 0.00% | 0.00% | 100.00% | 99.98% | 0.01% | 100.00% |
| Cas9-G1371-N and | | | | | | | | | |
| dSpCas9-G1371-C | | | | | | | | | |
| nogRNA-G1371spilt-KKH- | G | 99.97% | 99.98% | 99.98% | 99.93% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.00% | 0.00% | 0.00% | 0.04% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | A | 0.02% | 0.02% | 0.01% | 0.00% | 0.00% | 0.01% | 0.00% | 0.01% |
| Cas9-G1371-C and | | | | | | | | | |
| dSpCas9-G1371-N | | | | | | | | | |
| nogRNA-G1371spilt-KKH- | T | 0.00% | 0.01% | 0.01% | 0.01% | 0.01% | 0.01% | 99.95% | 0.02% |
| Cas9-G1371-C and | | | | | | | | | |
| dSpCas9-G1371-N | | | | | | | | | |
| nogRNA-G1371spilt-KKH- | C | 0.00% | 0.00% | 0.00% | 0.00% | 99.97% | 99.96% | 0.01% | 99.95% |
| Cas9-G1371-C and | | | | | | | | | |
| dSpCas9-G1371-N | | | | | | | | | |
| nogRNA-G1371spilt-KKH- | G | 99.96% | 99.94% | 99.96% | 99.94% | 0.00% | 0.00% | 0.01% | 0.00% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1371spilt-KKH- | — | 0.02% | 0.02% | 0.02% | 0.05% | 0.02% | 0.02% | 0.02% | 0.02% |
| Cas9-G1371-C and | | | | | | | | | |
| dSpCas9-G1371-N | | | | | | | | | |
| nogRNA-G1397spilt-KKH- | A | 0.03% | 0.01% | 0.00% | 0.00% | 0.00% | 0.00% | 0.02% | 0.00% |
| Cas9-G1397-N and | | | | | | | | | |
| dSpCas9-G1397-C | | | | | | | | | |
| nogRNA-G1397spilt-KKH- | T | 0.02% | 0.03% | 0.01% | 0.01% | 0.01% | 0.01% | 99.64% | 0.00% |
| Cas9-G1397-N and | | | | | | | | | |
| dSpCas9-G1397-C | | | | | | | | | |
| nogRNA-G1397spilt-KKH- | C | 0.00% | 0.00% | 0.00% | 0.00% | 99.66% | 99.66% | 0.01% | 99.67% |
| Cas9-G1397-N and | | | | | | | | | |
| dSpCas9-G1397-C | | | | | | | | | |
| nogRNA-G1397spilt-KKH- | G | 99.67% | 99.67% | 99.66% | 99.61% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.28% | 0.29% | 0.32% | 0.37% | 0.32% | 0.32% | 0.33% | 0.33% |
| Cas9-G1397-N and | | | | | | | | | |
| dSpCas9-G1397-C | | | | | | | | | |
| nogRNA-G1397spilt-KKH- | A | 0.01% | 0.02% | 0.01% | 0.02% | 0.01% | 0.01% | 0.01% | 0.00% |
| Cas9-G1397-C and | | | | | | | | | |
| dSpCas9-G1397-N | | | | | | | | | |
| nogRNA-G1397spilt-KKH- | T | 0.01% | 0.01% | 0.00% | 0.01% | 0.00% | 0.01% | 99.94% | 0.02% |
| Cas9-G1397-C and | | | | | | | | | |
| dSpCas9-G1397-N | | | | | | | | | |
| nogRNA-G1397spilt-KKH- | C | 0.00% | 0.00% | 0.00% | 0.00% | 99.97% | 99.96% | 0.02% | 99.96% |
| Cas9-G1397-C and | | | | | | | | | |
| dSpCas9-G1397-N | | | | | | | | | |
| nogRNA-G1397spilt-KKH- | G | 99.96% | 99.94% | 99.96% | 99.93% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.02% | 0.02% | 0.02% | 0.04% | 0.02% | 0.02% | 0.01% | 0.01% |
| Cas9-G1397-C and | | | | | | | | | |
| dSpCas9-G1397-N | | | | | | | | | |
| nogRNA-N1357spilt-KKH- | A | 0.02% | 0.00% | 0.01% | 0.00% | 0.00% | 0.00% | 0.00% | 0.01% |
| Cas9-N1357-N and | | | | | | | | | |
| dSpCas9-N1357-C | | | | | | | | | |
| nogRNA-N1357spilt-KKH- | T | 0.02% | 0.00% | 0.02% | 0.00% | 0.02% | 0.02% | 99.99% | 0.01% |
| Cas9-N1357-N and | | | | | | | | | |
| dSpCas9-N1357-C | | | | | | | | | |
| nogRNA-N1357spilt-KKH- | C | 0.00% | 0.00% | 0.00% | 0.00% | 99.98% | 99.98% | 0.01% | 99.98% |
| Cas9-N1357-N and | | | | | | | | | |
| dSpCas9-N1357-C | | | | | | | | | |
| nogRNA-N1357spilt-KKH- | G | 99.96% | 99.99% | 99.97% | 99.93% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.00% | 0.00% | 0.00% | 0.06% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | A | 0.00% | 0.01% | 0.01% | 0.02% | 0.02% | 0.00% | 0.01% | 0.00% |
| Cas9-N1357-C and | | | | | | | | | |
| dSpCas9-N1357-N | | | | | | | | | |
| nogRNA-N1357spilt-KKH- | T | 0.01% | 0.02% | 0.00% | 0.01% | 0.03% | 0.03% | 99.97% | 0.00% |
| Cas9-N1357-C and | | | | | | | | | |
| dSpCas9-N1357-N | | | | | | | | | |
| nogRNA-N1357spilt-KKH- | C | 0.01% | 0.01% | 0.00% | 0.00% | 99.95% | 99.97% | 0.02% | 100.00% |
| Cas9-N1357-C and | | | | | | | | | |
| dSpCas9-N1357-N | | | | | | | | | |
| nogRNA-N1357spilt-KKH- | G | 99.99% | 99.97% | 99.99% | 99.93% | 0.01% | 0.00% | 0.00% | 0.00% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.00% | 0.00% | 0.00% | 0.05% | 0.00% | 0.00% | 0.00% | 0.00% |
| Cas9-N1357-C and | | | | | | | | | |
| dSpCas9-N1357-N | | | | | | | | | |
| nogRNA-N1387spilt-KKH- | A | 0.01% | 0.01% | 0.02% | 0.02% | 0.00% | 0.01% | 0.00% | 0.00% |
| Cas9-N1387-N and | | | | | | | | | |
| dSpCas9-N1387-C | | | | | | | | | |
| nogRNA-N1387spilt-KKH- | T | 0.02% | 0.01% | 0.01% | 0.01% | 0.01% | 0.00% | 99.98% | 0.02% |
| Cas9-N1387-N and | | | | | | | | | |
| dSpCas9-N1387-C | | | | | | | | | |
| nogRNA-N1387spilt-KKH- | C | 0.01% | 0.00% | 0.00% | 0.00% | 99.98% | 99.97% | 0.00% | 99.97% |
| Cas9-N1387-N and | | | | | | | | | |
| dSpCas9-N1387-C | | | | | | | | | |
| nogRNA-N1387spilt-KKH- | G | 99.95% | 99.96% | 99.96% | 99.94% | 0.00% | 0.00% | 0.01% | 0.00% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.01% | 0.01% | 0.01% | 0.03% | 0.01% | 0.01% | 0.01% | 0.01% |
| Cas9-N1387-N and | | | | | | | | | |
| dSpCas9-N1387-C | | | | | | | | | |
| nogRNA-N1387spilt-KKH- | A | 0.01% | 0.02% | 0.01% | 0.01% | 0.01% | 0.01% | 0.00% | 0.00% |
| Cas9-N1387-C and | | | | | | | | | |
| dSpCas9-N1387-N | | | | | | | | | |
| nogRNA-N1387spilt-KKH- | T | 0.04% | 0.02% | 0.00% | 0.01% | 0.01% | 0.00% | 99.96% | 0.01% |
| Cas9-N1387-C and | | | | | | | | | |
| dSpCas9-N1387-N | | | | | | | | | |
| nogRNA-N1387spilt-KKH- | C | 0.00% | 0.01% | 0.00% | 0.00% | 99.97% | 99.98% | 0.02% | 99.98% |
| Cas9-N1387-C and | | | | | | | | | |
| dSpCas9-N1387-N | | | | | | | | | |
| nogRNA-N1387spilt-KKH- | G | 99.94% | 99.93% | 99.97% | 99.93% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.01% | 0.01% | 0.01% | 0.05% | 0.01% | 0.01% | 0.01% | 0.01% |
| Cas9-N1387-C and | | | | | | | | | |
| dSpCas9-N1387-N | | | | | | | | | |

| Batch | Nucleotide | C | T | G | A | G | T | T |
|---|---|---|---|---|---|---|---|---|
| nogRNA-A1343spilt-KKH- | A | 0.00% | 0.01% | 0.02% | 99.94% | 0.00% | 0.00% | 0.00% |
| Cas9-A1343-N and | | | | | | | | |
| dSpCas9-A1343-C | | | | | | | | |
| nogRNA-A1343spilt-KKH- | T | 0.02% | 99.98% | 0.01% | 0.01% | 0.01% | 99.98% | 99.99% |
| Cas9-A1343-N and | | | | | | | | |
| dSpCas9-A1343-C | | | | | | | | |
| nogRNA-A1343spilt-KKH- | C | 99.98% | 0.01% | 0.00% | 0.03% | 0.00% | 0.02% | 0.01% |
| Cas9-A1343-N and | | | | | | | | |
| dSpCas9-A1343-C | | | | | | | | |
| nogRNA-A1343spilt-KKH- | G | 0.00% | 0.00% | 99.97% | 0.02% | 99.99% | 0.01% | 0.00% |
| Cas9-A1343-N and | | | | | | | | |
| dSpCas9-A1343-C | | | | | | | | |
| nogRNA-A1343spilt-KKH- | N | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | — | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | A | 0.01% | 0.00% | 0.02% | 99.91% | 0.00% | 0.00% | 0.00% |
| Cas9-A1343-C and | | | | | | | | |
| dSpCas9-A1343-N | | | | | | | | |
| nogRNA-A1343spilt-KKH- | T | 0.01% | 99.96% | 0.02% | 0.00% | 0.00% | 100.00% | 100.00% |
| Cas9-A1343-C and | | | | | | | | |
| dSpCas9-A1343-N | | | | | | | | |
| nogRNA-A1343spilt-KKH- | C | 99.98% | 0.03% | 0.00% | 0.05% | 0.00% | 0.00% | 0.00% |
| Cas9-A1343-C and | | | | | | | | |
| dSpCas9-A1343-N | | | | | | | | |
| nogRNA-A1343spilt-KKH- | G | 0.00% | 0.00% | 99.96% | 0.04% | 100.00% | 0.00% | 0.00% |
| Cas9-A1343-C and | | | | | | | | |
| dSpCas9-A1343-N | | | | | | | | |
| nogRNA-A1343spilt-KKH- | N | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-A1343spilt-KKH- | — | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-G1322spilt-KKH- | A | 0.01% | 0.00% | 0.02% | 99.95% | 0.01% | 0.00% | 0.00% |
| Cas9-G1322-N and | | | | | | | | |
| dSpCas9-G1322-C | | | | | | | | |
| nogRNA-G1322spilt-KKH- | T | 0.01% | 99.97% | 0.01% | 0.01% | 0.00% | 99.98% | 99.97% |
| Cas9-G1322-N and | | | | | | | | |
| dSpCas9-G1322-C | | | | | | | | |
| nogRNA-G1322spilt-KKH- | C | 99.98% | 0.03% | 0.00% | 0.02% | 0.00% | 0.01% | 0.02% |
| Cas9-G1322-N and | | | | | | | | |
| dSpCas9-G1322-C | | | | | | | | |
| nogRNA-G1322spilt-KKH- | G | 0.00% | 0.00% | 99.97% | 0.03% | 99.99% | 0.01% | 0.01% |
| Cas9-G1322-N and | | | | | | | | |
| dSpCas9-G1322-C | | | | | | | | |
| nogRNA-G1322spilt-KKH- | N | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | — | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | A | 0.01% | 0.00% | 0.02% | 99.93% | 0.00% | 0.00% | 0.00% |
| Cas9-G1322-C and | | | | | | | | |
| dSpCas9-G1322-N | | | | | | | | |
| nogRNA-G1322spilt-KKH- | T | 0.00% | 99.97% | 0.02% | 0.00% | 0.00% | 99.96% | 99.99% |
| Cas9-G1322-C and | | | | | | | | |
| dSpCas9-G1322-N | | | | | | | | |
| nogRNA-G1322spilt-KKH- | C | 99.99% | 0.02% | 0.00% | 0.03% | 0.00% | 0.01% | 0.00% |
| Cas9-G1322-C and | | | | | | | | |
| dSpCas9-G1322-N | | | | | | | | |
| nogRNA-G1322spilt-KKH- | G | 0.00% | 0.00% | 99.96% | 0.04% | 100.00% | 0.03% | 0.00% |
| Cas9-G1322-C and | | | | | | | | |
| dSpCas9-G1322-N | | | | | | | | |
| nogRNA-G1322spilt-KKH- | N | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1322spilt-KKH- | — | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1333spilt-KKH- | A | 0.00% | 0.01% | 0.01% | 99.96% | 0.00% | 0.01% | 0.00% |
| Cas9-G1333-N and | | | | | | | | |
| dSpCas9-G1333-C | | | | | | | | |
| nogRNA-G1333spilt-KKH- | T | 0.00% | 99.94% | 0.00% | 0.01% | 0.00% | 99.96% | 99.98% |
| Cas9-G1333-N and | | | | | | | | |
| dSpCas9-G1333-C | | | | | | | | |
| nogRNA-G1333spilt-KKH- | C | 99.98% | 0.04% | 0.00% | 0.01% | 0.00% | 0.02% | 0.01% |
| Cas9-G1333-N and | | | | | | | | |
| dSpCas9-G1333-C | | | | | | | | |
| nogRNA-G1333spilt-KKH- | G | 0.00% | 0.00% | 99.98% | 0.01% | 100.00% | 0.01% | 0.00% |
| Cas9-G1333-N and | | | | | | | | |
| dSpCas9-G1333-C | | | | | | | | |
| nogRNA-G1333spilt-KKH- | N | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | — | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | A | 0.00% | 0.01% | 0.00% | 99.90% | 0.00% | 0.00% | 0.00% |
| Cas9-G1333-C and | | | | | | | | |
| dSpCas9-G1333-N | | | | | | | | |
| nogRNA-G1333spilt-KKH- | T | 0.01% | 99.98% | 0.01% | 0.00% | 0.00% | 99.99% | 99.98% |
| Cas9-G1333-C and | | | | | | | | |
| dSpCas9-G1333-N | | | | | | | | |
| nogRNA-G1333spilt-KKH- | C | 99.99% | 0.01% | 0.00% | 0.05% | 0.00% | 0.01% | 0.02% |
| Cas9-G1333-C and | | | | | | | | |
| dSpCas9-G1333-N | | | | | | | | |
| nogRNA-G1333spilt-KKH- | G | 0.00% | 0.00% | 99.98% | 0.05% | 100.00% | 0.00% | 0.00% |
| Cas9-G1333-C and | | | | | | | | |
| dSpCas9-G1333-N | | | | | | | | |
| nogRNA-G1333spilt-KKH- | N | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1333spilt-KKH- | — | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1371spilt-KKH- | A | 0.01% | 0.00% | 0.02% | 99.89% | 0.01% | 0.00% | 0.00% |
| Cas9-G1371-N and | | | | | | | | |
| dSpCas9-G1371-C | | | | | | | | |
| nogRNA-G1371spilt-KKH- | T | 0.00% | 99.98% | 0.01% | 0.01% | 0.00% | 99.98% | 99.99% |
| Cas9-G1371-N and | | | | | | | | |
| dSpCas9-G1371-C | | | | | | | | |
| nogRNA-G1371spilt-KKH- | C | 99.98% | 0.01% | 0.00% | 0.06% | 0.00% | 0.01% | 0.00% |
| Cas9-G1371-N and | | | | | | | | |
| dSpCas9-G1371-C | | | | | | | | |
| nogRNA-G1371spilt-KKH- | G | 0.00% | 0.00% | 99.97% | 0.04% | 99.99% | 0.01% | 0.00% |
| Cas9-G1371-N and | | | | | | | | |
| dSpCas9-G1371-C | | | | | | | | |
| nogRNA-G1371spilt-KKH- | N | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | — | 0.01% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | A | 0.00% | 0.00% | 0.01% | 99.92% | 0.00% | 0.00% | 0.00% |
| Cas9-G1371-C and | | | | | | | | |
| dSpCas9-G1371-N | | | | | | | | |
| nogRNA-G1371spilt-KKH- | T | 0.01% | 99.96% | 0.01% | 0.01% | 0.00% | 99.94% | 99.95% |
| Cas9-G1371-C and | | | | | | | | |
| dSpCas9-G1371-N | | | | | | | | |
| nogRNA-G1371spilt-KKH- | C | 99.97% | 0.00% | 0.00% | 0.03% | 0.00% | 0.02% | 0.01% |
| Cas9-G1371-C and | | | | | | | | |
| dSpCas9-G1371-N | | | | | | | | |
| nogRNA-G1371spilt-KKH- | G | 0.00% | 0.01% | 99.96% | 0.02% | 99.97% | 0.00% | 0.00% |
| Cas9-G1371-C and | | | | | | | | |
| dSpCas9-G1371-N | | | | | | | | |
| nogRNA-G1371spilt-KKH- | N | 0.00% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1371spilt-KKH- | — | 0.02% | 0.02% | 0.02% | 0.03% | 0.03% | 0.03% | 0.03% |
| Cas9-G1371-C and | | | | | | | | |
| dSpCas9-G1371-N | | | | | | | | |
| nogRNA-G1397spilt-KKH- | A | 0.00% | 0.00% | 0.01% | 99.60% | 0.00% | 0.00% | 0.00% |
| Cas9-G1397-N and | | | | | | | | |
| dSpCas9-G1397-C | | | | | | | | |
| nogRNA-G1397spilt-KKH- | T | 0.01% | 99.64% | 0.01% | 0.00% | 0.00% | 99.66% | 99.65% |
| Cas9-G1397-N and | | | | | | | | |
| dSpCas9-G1397-C | | | | | | | | |
| nogRNA-G1397spilt-KKH- | C | 99.66% | 0.02% | 0.00% | 0.03% | 0.00% | 0.00% | 0.02% |
| Cas9-G1397-N and | | | | | | | | |
| dSpCas9-G1397-C | | | | | | | | |
| nogRNA-G1397spilt-KKH- | G | 0.00% | 0.01% | 99.64% | 0.03% | 99.66% | 0.01% | 0.00% |
| Cas9-G1397-N and | | | | | | | | |
| dSpCas9-G1397-C | | | | | | | | |
| nogRNA-G1397spilt-KKH- | N | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | — | 0.33% | 0.33% | 0.33% | 0.34% | 0.34% | 0.33% | 0.33% |
| Cas9-G1397-N and | | | | | | | | |
| dSpCas9-G1397-C | | | | | | | | |
| nogRNA-G1397spilt-KKH- | A | 0.01% | 0.00% | 0.00% | 99.93% | 0.00% | 0.00% | 0.00% |
| Cas9-G1397-C and | | | | | | | | |
| dSpCas9-G1397-N | | | | | | | | |
| nogRNA-G1397spilt-KKH- | T | 0.01% | 99.98% | 0.01% | 0.00% | 0.00% | 99.95% | 99.99% |
| Cas9-G1397-C and | | | | | | | | |
| dSpCas9-G1397-N | | | | | | | | |
| nogRNA-G1397spilt-KKH- | C | 99.97% | 0.00% | 0.00% | 0.03% | 0.00% | 0.02% | 0.00% |
| Cas9-G1397-C and | | | | | | | | |
| dSpCas9-G1397-N | | | | | | | | |
| nogRNA-G1397spilt-KKH- | G | 0.00% | 0.00% | 99.98% | 0.02% | 99.99% | 0.02% | 0.00% |
| Cas9-G1397-C and | | | | | | | | |
| dSpCas9-G1397-N | | | | | | | | |
| nogRNA-G1397spilt-KKH- | N | 0.00% | 0.00% | 0.00% | 0.00% | 0.00% | 0.00% | 0.00% |
| Cas9-G1397-C and | | | | | | | | |
| dSpCas9-G1397-N | | | | | | | | |
| nogRNA-G1397spilt-KKH- | — | 0.01% | 0.01% | 0.01% | 0.01% | 0.01% | 0.01% | 0.01% |
| Cas9-G1397-C and | | | | | | | | |
| dSpCas9-G1397-N | | | | | | | | |
| nogRNA-N1357spilt-KKH- | A | 0.00% | 0.01% | 0.02% | 99.95% | 0.01% | 0.00% | 0.00% |
| Cas9-N1357-N and | | | | | | | | |
| dSpCas9-N1357-C | | | | | | | | |
| nogRNA-N1357spilt-KKH- | T | 0.01% | 99.97% | 0.01% | 0.01% | 0.00% | 99.98% | 100.00% |
| Cas9-N1357-N and | | | | | | | | |
| dSpCas9-N1357-C | | | | | | | | |
| nogRNA-N1357spilt-KKH- | C | 99.99% | 0.02% | 0.00% | 0.02% | 0.00% | 0.00% | 0.00% |
| Cas9-N1357-N and | | | | | | | | |
| dSpCas9-N1357-C | | | | | | | | |
| nogRNA-N1357spilt-KKH- | G | 0.00% | 0.00% | 99.98% | 0.03% | 99.99% | 0.02% | 0.00% |
| Cas9-N1357-N and | | | | | | | | |
| dSpCas9-N1357-C | | | | | | | | |
| nogRNA-N1357spilt-KKH- | N | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | — | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | A | 0.00% | 0.00% | 0.01% | 99.88% | 0.00% | 0.01% | 0.00% |
| Cas9-N1357-C and | | | | | | | | |
| dSpCas9-N1357-N | | | | | | | | |
| nogRNA-N1357spilt-KKH- | T | 0.01% | 99.98% | 0.02% | 0.01% | 0.01% | 99.97% | 99.99% |
| Cas9-N1357-C and | | | | | | | | |
| dSpCas9-N1357-N | | | | | | | | |
| nogRNA-N1357spilt-KKH- | C | 99.99% | 0.02% | 0.00% | 0.08% | 0.00% | 0.00% | 0.01% |
| Cas9-N1357-C and | | | | | | | | |
| dSpCas9-N1357-N | | | | | | | | |
| nogRNA-N1357spilt-KKH- | G | 0.00% | 0.01% | 99.97% | 0.03% | 99.99% | 0.02% | 0.00% |
| Cas9-N1357-C and | | | | | | | | |
| dSpCas9-N1357-N | | | | | | | | |
| nogRNA-N1357spilt-KKH- | N | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1357spilt-KKH- | — | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1387spilt-KKH- | A | 0.00% | 0.01% | 0.02% | 99.89% | 0.01% | 0.00% | 0.00% |
| Cas9-N1387-N and | | | | | | | | |
| dSpCas9-N1387-C | | | | | | | | |
| nogRNA-N1387spilt-KKH- | T | 0.01% | 99.96% | 0.01% | 0.01% | 0.00% | 99.97% | 99.97% |
| Cas9-N1387-N and | | | | | | | | |
| dSpCas9-N1387-C | | | | | | | | |
| nogRNA-N1387spilt-KKH- | C | 99.97% | 0.02% | 0.00% | 0.04% | 0.00% | 0.01% | 0.01% |
| Cas9-N1387-N and | | | | | | | | |
| dSpCas9-N1387-C | | | | | | | | |
| nogRNA-N1387spilt-KKH- | G | 0.00% | 0.01% | 99.96% | 0.05% | 99.98% | 0.00% | 0.00% |
| Cas9-N1387-N and | | | | | | | | |
| dSpCas9-N1387-C | | | | | | | | |
| nogRNA-N1387spilt-KKH- | N | 0.00% | 0.00% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | — | 0.01% | 0.01% | 0.01% | 0.01% | 0.01% | 0.01% | 0.02% |
| Cas9-N1387-N and | | | | | | | | |
| dSpCas9-N1387-C | | | | | | | | |
| nogRNA-N1387spilt-KKH- | A | 0.00% | 0.00% | 0.01% | 99.92% | 0.01% | 0.00% | 0.00% |
| Cas9-N1387-C and | | | | | | | | |
| dSpCas9-N1387-N | | | | | | | | |
| nogRNA-N1387spilt-KKH- | T | 0.01% | 99.95% | 0.00% | 0.00% | 0.00% | 99.96% | 99.97% |
| Cas9-N1387-C and | | | | | | | | |
| dSpCas9-N1387-N | | | | | | | | |
| nogRNA-N1387spilt-KKH- | C | 99.97% | 0.03% | 0.00% | 0.02% | 0.00% | 0.01% | 0.02% |
| Cas9-N1387-C and | | | | | | | | |
| dSpCas9-N1387-N | | | | | | | | |
| nogRNA-N1387spilt-KKH- | G | 0.00% | 0.00% | 99.98% | 0.04% | 99.97% | 0.00% | 0.00% |
| Cas9-N1387-C and | | | | | | | | |
| dSpCas9-N1387-N | | | | | | | | |
| nogRNA-N1387spilt-KKH- | N | 0.00% | 0.00% | 0.00% | 0.00% | 0.00% | 0.00% | 0.00% |
| Cas9-N1387-C and | | | | | | | | |
| dSpCas9-N1387-N | | | | | | | | |
| nogRNA-N1387spilt-KKH- | — | 0.01% | 0.01% | 0.01% | 0.01% | 0.01% | 0.01% | 0.01% |
| Cas9-N1387-C and | | | | | | | | |
| dSpCas9-N1387-N | | | | | | | | |

| Batch | Nucleotide | T | C | T | C | A | T | C | T | G |
|---|---|---|---|---|---|---|---|---|---|---|
| nogRNA-A1343spilt-KKH- | A | 0.00% | 0.00% | 0.01% | 0.01% | 99.95% | 0.00% | 0.00% | 0.00% | 0.01% |
| Cas9-A1343-N and | | | | | | | | | | |
| dSpCas9-A1343-C | | | | | | | | | | |
| nogRNA-A1343spilt-KKH- | T | 99.99% | 0.01% | 99.97% | 0.01% | 0.01% | 99.98% | 0.01% | 99.99% | 0.00% |
| Cas9-A1343-N and | | | | | | | | | | |
| dSpCas9-A1343-C | | | | | | | | | | |
| nogRNA-A1343spilt-KKH- | C | 0.01% | 99.98% | 0.01% | 99.98% | 0.00% | 0.01% | 99.99% | 0.01% | 0.00% |
| Cas9-A1343-N and | | | | | | | | | | |
| dSpCas9-A1343-C | | | | | | | | | | |
| nogRNA-A1343spilt-KKH- | G | 0.00% | 0.00% | 0.01% | 0.00% | 0.04% | 0.00% | 0.00% | 0.00% | 99.99% |
| Cas9-A1343-N and | | | | | | | | | | |
| dSpCas9-A1343-C | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | A | 0.00% | 0.00% | 0.01% | 0.01% | 99.95% | 0.00% | 0.01% | 0.01% | 0.02% |
| Cas9-A1343-C and | | | | | | | | | | |
| dSpCas9-A1343-N | | | | | | | | | | |
| nogRNA-A1343spilt-KKH- | T | 100.00% | 0.01% | 99.97% | 0.03% | 0.00% | 100.00% | 0.00% | 99.97% | 0.01% |
| Cas9-A1343-C and | | | | | | | | | | |
| dSpCas9-A1343-N | | | | | | | | | | |
| nogRNA-A1343spilt-KKH- | C | 0.00% | 99.99% | 0.01% | 99.96% | 0.04% | 0.00% | 99.97% | 0.02% | 0.00% |
| Cas9-A1343-C and | | | | | | | | | | |
| dSpCas9-A1343-N | | | | | | | | | | |
| nogRNA-A1343spilt-KKH- | G | 0.00% | 0.00% | 0.00% | 0.00% | 0.01% | 0.00% | 0.01% | 0.00% | 99.97% |
| Cas9-A1343-C and | | | | | | | | | | |
| dSpCas9-A1343-N | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-G1322spilt-KKH- | A | 0.00% | 0.00% | 0.01% | 0.01% | 99.96% | 0.00% | 0.00% | 0.01% | 0.00% |
| Cas9-G1322-N and | | | | | | | | | | |
| dSpCas9-G1322-C | | | | | | | | | | |
| nogRNA-G1322spilt-KKH- | T | 99.99% | 0.00% | 99.97% | 0.01% | 0.00% | 99.99% | 0.00% | 99.96% | 0.02% |
| Cas9-G1322-N and | | | | | | | | | | |
| dSpCas9-G1322-C | | | | | | | | | | |
| nogRNA-G1322spilt-KKH- | C | 0.01% | 99.99% | 0.02% | 99.97% | 0.01% | 0.01% | 99.99% | 0.02% | 0.00% |
| Cas9-G1322-N and | | | | | | | | | | |
| dSpCas9-G1322-C | | | | | | | | | | |
| nogRNA-G1322spilt-KKH- | G | 0.00% | 0.00% | 0.00% | 0.00% | 0.03% | 0.00% | 0.00% | 0.00% | 99.98% |
| Cas9-G1322-N and | | | | | | | | | | |
| dSpCas9-G1322-C | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | A | 0.00% | 0.00% | 0.01% | 0.00% | 99.96% | 0.00% | 0.01% | 0.00% | 0.00% |
| Cas9-G1322-C and | | | | | | | | | | |
| dSpCas9-G1322-N | | | | | | | | | | |
| nogRNA-G1322spilt-KKH- | T | 99.97% | 0.00% | 99.98% | 0.01% | 0.00% | 99.98% | 0.01% | 99.97% | 0.01% |
| Cas9-G1322-C and | | | | | | | | | | |
| dSpCas9-G1322-N | | | | | | | | | | |
| nogRNA-G1322spilt-KKH- | C | 0.02% | 100.00% | 0.00% | 99.98% | 0.00% | 0.01% | 99.97% | 0.01% | 0.00% |
| Cas9-G1322-C and | | | | | | | | | | |
| dSpCas9-G1322-N | | | | | | | | | | |
| nogRNA-G1322spilt-KKH- | G | 0.00% | 0.00% | 0.00% | 0.00% | 0.03% | 0.00% | 0.00% | 0.01% | 99.97% |
| Cas9-G1322-C and | | | | | | | | | | |
| dSpCas9-G1322-N | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1322spilt-KKH- | — | 0.01% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1333spilt-KKH- | A | 0.00% | 0.00% | 0.00% | 0.01% | 99.95% | 0.00% | 0.00% | 0.01% | 0.01% |
| Cas9-G1333-N and | | | | | | | | | | |
| dSpCas9-G1333-C | | | | | | | | | | |
| nogRNA-G1333spilt-KKH- | T | 99.97% | 0.01% | 99.96% | 0.01% | 0.00% | 99.99% | 0.02% | 99.97% | 0.01% |
| Cas9-G1333-N and | | | | | | | | | | |
| dSpCas9-G1333-C | | | | | | | | | | |
| nogRNA-G1333spilt-KKH- | C | 0.01% | 99.98% | 0.03% | 99.97% | 0.00% | 0.00% | 99.97% | 0.02% | 0.00% |
| Cas9-G1333-N and | | | | | | | | | | |
| dSpCas9-G1333-C | | | | | | | | | | |
| nogRNA-G1333spilt-KKH- | G | 0.01% | 0.00% | 0.01% | 0.00% | 0.04% | 0.00% | 0.00% | 0.00% | 99.97% |
| Cas9-G1333-N and | | | | | | | | | | |
| dSpCas9-G1333-C | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | A | 0.00% | 0.00% | 0.02% | 0.00% | 99.98% | 0.00% | 0.00% | 0.02% | 0.01% |
| Cas9-G1333-C and | | | | | | | | | | |
| dSpCas9-G1333-N | | | | | | | | | | |
| nogRNA-G1333spilt-KKH- | T | 99.97% | 0.01% | 99.97% | 0.03% | 0.00% | 99.98% | 0.01% | 99.96% | 0.01% |
| Cas9-G1333-C and | | | | | | | | | | |
| dSpCas9-G1333-N | | | | | | | | | | |
| nogRNA-G1333spilt-KKH- | C | 0.02% | 99.99% | 0.01% | 99.97% | 0.00% | 0.01% | 99.99% | 0.02% | 0.00% |
| Cas9-G1333-C and | | | | | | | | | | |
| dSpCas9-G1333-N | | | | | | | | | | |
| nogRNA-G1333spilt-KKH- | G | 0.01% | 0.00% | 0.00% | 0.00% | 0.02% | 0.00% | 0.00% | 0.00% | 99.98% |
| Cas9-G1333-C and | | | | | | | | | | |
| dSpCas9-G1333-N | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1371spilt-KKH- | A | 0.00% | 0.00% | 0.02% | 0.01% | 99.94% | 0.00% | 0.02% | 0.01% | 0.01% |
| Cas9-G1371-N and | | | | | | | | | | |
| dSpCas9-G1371-C | | | | | | | | | | |
| nogRNA-G1371spilt-KKH- | T | 99.98% | 0.01% | 99.97% | 0.03% | 0.01% | 99.98% | 0.01% | 99.98% | 0.02% |
| Cas9-G1371-N and | | | | | | | | | | |
| dSpCas9-G1371-C | | | | | | | | | | |
| nogRNA-G1371spilt-KKH- | C | 0.02% | 99.98% | 0.02% | 99.96% | 0.00% | 0.01% | 99.97% | 0.01% | 0.00% |
| Cas9-G1371-N and | | | | | | | | | | |
| dSpCas9-G1371-C | | | | | | | | | | |
| nogRNA-G1371spilt-KKH- | G | 0.00% | 0.00% | 0.00% | 0.00% | 0.05% | 0.00% | 0.00% | 0.00% | 99.97% |
| Cas9-G1371-N and | | | | | | | | | | |
| dSpCas9-G1371-C | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | — | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | A | 0.00% | 0.00% | 0.01% | 0.00% | 99.94% | 0.00% | 0.00% | 0.00% | 0.02% |
| Cas9-G1371-C and | | | | | | | | | | |
| dSpCas9-G1371-N | | | | | | | | | | |
| nogRNA-G1371spilt-KKH- | T | 99.96% | 0.01% | 99.94% | 0.02% | 0.00% | 99.95% | 0.00% | 99.95% | 0.01% |
| Cas9-G1371-C and | | | | | | | | | | |
| dSpCas9-G1371-N | | | | | | | | | | |
| nogRNA-G1371spilt-KKH- | C | 0.00% | 99.96% | 0.02% | 99.94% | 0.01% | 0.01% | 99.96% | 0.01% | 0.00% |
| Cas9-G1371-C and | | | | | | | | | | |
| dSpCas9-G1371-N | | | | | | | | | | |
| nogRNA-G1371spilt-KKH- | G | 0.01% | 0.00% | 0.00% | 0.00% | 0.01% | 0.00% | 0.00% | 0.00% | 99.93% |
| Cas9-G1371-C and | | | | | | | | | | |
| dSpCas9-G1371-N | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1371spilt-KKH- | — | 0.03% | 0.03% | 0.03% | 0.03% | 0.04% | 0.04% | 0.04% | 0.03% | 0.03% |
| Cas9-G1371-C and | | | | | | | | | | |
| dSpCas9-G1371-N | | | | | | | | | | |
| nogRNA-G1397spilt-KKH- | A | 0.00% | 0.01% | 0.02% | 0.00% | 99.64% | 0.00% | 0.01% | 0.01% | 0.01% |
| Cas9-G1397-N and | | | | | | | | | | |
| dSpCas9-G1397-C | | | | | | | | | | |
| nogRNA-G1397spilt-KKH- | T | 99.66% | 0.01% | 99.63% | 0.00% | 0.01% | 99.65% | 0.01% | 99.65% | 0.00% |
| Cas9-G1397-N and | | | | | | | | | | |
| dSpCas9-G1397-C | | | | | | | | | | |
| nogRNA-G1397spilt-KKH- | C | 0.01% | 99.65% | 0.00% | 99.66% | 0.00% | 0.00% | 99.65% | 0.00% | 0.00% |
| Cas9-G1397-N and | | | | | | | | | | |
| dSpCas9-G1397-C | | | | | | | | | | |
| nogRNA-G1397spilt-KKH- | G | 0.00% | 0.00% | 0.01% | 0.00% | 0.02% | 0.01% | 0.00% | 0.00% | 99.65% |
| Cas9-G1397-N and | | | | | | | | | | |
| dSpCas9-G1397-C | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | — | 0.33% | 0.33% | 0.34% | 0.34% | 0.33% | 0.33% | 0.33% | 0.33% | 0.33% |
| Cas9-G1397-N and | | | | | | | | | | |
| dSpCas9-G1397-C | | | | | | | | | | |
| nogRNA-G1397spilt-KKH- | A | 0.00% | 0.00% | 0.01% | 0.00% | 99.94% | 0.00% | 0.01% | 0.00% | 0.02% |
| Cas9-G1397-C and | | | | | | | | | | |
| dSpCas9-G1397-N | | | | | | | | | | |
| nogRNA-G1397spilt-KKH- | T | 99.99% | 0.01% | 99.96% | 0.01% | 0.00% | 99.97% | 0.01% | 99.97% | 0.01% |
| Cas9-G1397-C and | | | | | | | | | | |
| dSpCas9-G1397-N | | | | | | | | | | |
| nogRNA-G1397spilt-KKH- | C | 0.00% | 99.98% | 0.01% | 99.98% | 0.01% | 0.01% | 99.97% | 0.01% | 0.00% |
| Cas9-G1397-C and | | | | | | | | | | |
| dSpCas9-G1397-N | | | | | | | | | | |
| nogRNA-G1397spilt-KKH- | G | 0.00% | 0.00% | 0.00% | 0.00% | 0.04% | 0.00% | 0.00% | 0.00% | 99.95% |
| Cas9-G1397-C and | | | | | | | | | | |
| dSpCas9-G1397-N | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-G1397spilt-KKH- | — | 0.01% | 0.01% | 0.01% | 0.01% | 0.01% | 0.01% | 0.01% | 0.02% | 0.02% |
| Cas9-G1397-C and | | | | | | | | | | |
| dSpCas9-G1397-N | | | | | | | | | | |
| nogRNA-N1357spilt-KKH- | A | 0.00% | 0.00% | 0.01% | 0.00% | 99.97% | 0.00% | 0.00% | 0.00% | 0.02% |
| Cas9-N1357-N and | | | | | | | | | | |
| dSpCas9-N1357-C | | | | | | | | | | |
| nogRNA-N1357spilt-KKH- | T | 99.98% | 0.02% | 99.97% | 0.02% | 0.01% | 99.99% | 0.01% | 99.98% | 0.02% |
| Cas9-N1357-N and | | | | | | | | | | |
| dSpCas9-N1357-C | | | | | | | | | | |
| nogRNA-N1357spilt-KKH- | C | 0.01% | 99.98% | 0.00% | 99.98% | 0.01% | 0.01% | 99.99% | 0.02% | 0.00% |
| Cas9-N1357-N and | | | | | | | | | | |
| dSpCas9-N1357-C | | | | | | | | | | |
| nogRNA-N1357spilt-KKH- | G | 0.00% | 0.00% | 0.01% | 0.00% | 0.02% | 0.00% | 0.00% | 0.00% | 99.97% |
| Cas9-N1357-N and | | | | | | | | | | |
| dSpCas9-N1357-C | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | A | 0.00% | 0.00% | 0.02% | 0.00% | 99.93% | 0.01% | 0.01% | 0.01% | 0.02% |
| Cas9-N1357-C and | | | | | | | | | | |
| dSpCas9-N1357-N | | | | | | | | | | |
| nogRNA-N1357spilt-KKH- | T | 99.99% | 0.01% | 99.97% | 0.02% | 0.00% | 99.99% | 0.03% | 99.98% | 0.01% |
| Cas9-N1357-C and | | | | | | | | | | |
| dSpCas9-N1357-N | | | | | | | | | | |
| nogRNA-N1357spilt-KKH- | C | 0.01% | 99.99% | 0.01% | 99.98% | 0.05% | 0.00% | 99.97% | 0.02% | 0.01% |
| Cas9-N1357-C and | | | | | | | | | | |
| dSpCas9-N1357-N | | | | | | | | | | |
| nogRNA-N1357spilt-KKH- | G | 0.00% | 0.00% | 0.01% | 0.00% | 0.02% | 0.00% | 0.00% | 0.00% | 99.97% |
| Cas9-N1357-C and | | | | | | | | | | |
| dSpCas9-N1357-N | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1387spilt-KKH- | A | 0.00% | 0.00% | 0.03% | 0.01% | 99.96% | 0.00% | 0.01% | 0.01% | 0.00% |
| Cas9-N1387-N and | | | | | | | | | | |
| dSpCas9-N1387-C | | | | | | | | | | |
| nogRNA-N1387spilt-KKH- | T | 99.97% | 0.00% | 99.94% | 0.04% | 0.00% | 99.96% | 0.04% | 99.96% | 0.01% |
| Cas9-N1387-N and | | | | | | | | | | |
| dSpCas9-N1387-C | | | | | | | | | | |
| nogRNA-N1387spilt-KKH- | C | 0.00% | 99.97% | 0.01% | 99.94% | 0.00% | 0.01% | 99.94% | 0.00% | 0.00% |
| Cas9-N1387-N and | | | | | | | | | | |
| dSpCas9-N1387-C | | | | | | | | | | |
| nogRNA-N1387spilt-KKH- | G | 0.00% | 0.00% | 0.00% | 0.00% | 0.01% | 0.01% | 0.00% | 0.00% | 99.97% |
| Cas9-N1387-N and | | | | | | | | | | |
| dSpCas9-N1387-C | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | — | 0.02% | 0.01% | 0.01% | 0.01% | 0.01% | 0.01% | 0.02% | 0.02% | 0.02% |
| Cas9-N1387-N and | | | | | | | | | | |
| dSpCas9-N1387-C | | | | | | | | | | |
| nogRNA-N1387spilt-KKH- | A | 0.00% | 0.00% | 0.00% | 0.00% | 99.97% | 0.00% | 0.00% | 0.01% | 0.01% |
| Cas9-N1387-C and | | | | | | | | | | |
| dSpCas9-N1387-N | | | | | | | | | | |
| nogRNA-N1387spilt-KKH- | T | 99.97% | 0.02% | 99.98% | 0.01% | 0.00% | 99.96% | 0.00% | 99.95% | 0.00% |
| Cas9-N1387-C and | | | | | | | | | | |
| dSpCas9-N1387-N | | | | | | | | | | |
| nogRNA-N1387spilt-KKH- | C | 0.01% | 99.96% | 0.00% | 99.97% | 0.00% | 0.02% | 99.98% | 0.03% | 0.00% |
| Cas9-N1387-C and | | | | | | | | | | |
| dSpCas9-N1387-N | | | | | | | | | | |
| nogRNA-N1387spilt-KKH- | G | 0.00% | 0.01% | 0.00% | 0.00% | 0.00% | 0.00% | 0.00% | 0.00% | 99.97% |
| Cas9-N1387-C and | | | | | | | | | | |
| dSpCas9-N1387-N | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |
| nogRNA-N1387spilt-KKH- | — | 0.01% | 0.01% | 0.01% | 0.01% | 0.01% | 0.01% | 0.01% | 0.01% | 0.01% |
| Cas9-N1387-C and | | | | | | | | | | |
| dSpCas9-N1387-N | | | | | | | | | | |

| Batch | Nucleotide | T | G | C | C | C | C | T | C |
|---|---|---|---|---|---|---|---|---|---|
| nogRNA-A1343spilt-KKH- | A | 0.00% | 0.03% | 0.00% | 0.02% | 0.01% | 0.02% | 0.01% | 0.02% |
| Cas9-A1343-N and | | | | | | | | | |
| dSpCas9-A1343-C | | | | | | | | | |
| nogRNA-A1343spilt-KKH- | T | 99.98% | 0.01% | 0.01% | 0.01% | 0.02% | 0.01% | 99.96% | 0.02% |
| Cas9-A1343-N and | | | | | | | | | |
| dSpCas9-A1343-C | | | | | | | | | |
| nogRNA-A1343spilt-KKH- | C | 0.02% | 0.01% | 99.99% | 99.98% | 99.97% | 99.95% | 0.02% | 99.97% |
| Cas9-A1343-N and | | | | | | | | | |
| dSpCas9-A1343-C | | | | | | | | | |
| nogRNA-A1343spilt-KKH- | G | 0.00% | 99.95% | 0.00% | 0.00% | 0.00% | 0.00% | 0.01% | 0.00% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.00% | 0.00% | 0.00% | 0.00% | 0.00% | 0.01% | 0.00% | 0.00% |
| Cas9-A1343-N and | | | | | | | | | |
| dSpCas9-A1343-C | | | | | | | | | |
| nogRNA-A1343spilt-KKH- | A | 0.00% | 0.03% | 0.00% | 0.00% | 0.01% | 0.01% | 0.00% | 0.03% |
| Cas9-A1343-C and | | | | | | | | | |
| dSpCas9-A1343-N | | | | | | | | | |
| nogRNA-A1343spilt-KKH- | T | 99.97% | 0.00% | 0.01% | 0.01% | 0.00% | 0.02% | 99.96% | 0.01% |
| Cas9-A1343-C and | | | | | | | | | |
| dSpCas9-A1343-N | | | | | | | | | |
| nogRNA-A1343spilt-KKH- | C | 0.02% | 0.00% | 99.98% | 99.98% | 99.99% | 99.97% | 0.03% | 99.96% |
| Cas9-A1343-C and | | | | | | | | | |
| dSpCas9-A1343-N | | | | | | | | | |
| nogRNA-A1343spilt-KKH- | G | 0.01% | 99.96% | 0.00% | 0.00% | 0.00% | 0.00% | 0.01% | 0.00% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-G1322spilt-KKH- | A | 0.02% | 0.02% | 0.00% | 0.01% | 0.02% | 0.02% | 0.02% | 0.00% |
| Cas9-G1322-N and | | | | | | | | | |
| dSpCas9-G1322-C | | | | | | | | | |
| nogRNA-G1322spilt-KKH- | T | 99.96% | 0.02% | 0.01% | 0.01% | 0.01% | 0.00% | 99.94% | 0.06% |
| Cas9-G1322-N and | | | | | | | | | |
| dSpCas9-G1322-C | | | | | | | | | |
| nogRNA-G1322spilt-KKH- | C | 0.02% | 0.01% | 99.99% | 99.96% | 99.97% | 99.98% | 0.02% | 99.94% |
| Cas9-G1322-N and | | | | | | | | | |
| dSpCas9-G1322-C | | | | | | | | | |
| nogRNA-G1322spilt-KKH- | G | 0.00% | 99.96% | 0.00% | 0.01% | 0.00% | 0.00% | 0.01% | 0.00% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | A | 0.01% | 0.01% | 0.00% | 0.01% | 0.02% | 0.01% | 0.01% | 0.01% |
| Cas9-G1322-C and | | | | | | | | | |
| dSpCas9-G1322-N | | | | | | | | | |
| nogRNA-G1322spilt-KKH- | T | 99.95% | 0.00% | 0.04% | 0.02% | 0.03% | 0.03% | 99.97% | 0.02% |
| Cas9-G1322-C and | | | | | | | | | |
| dSpCas9-G1322-N | | | | | | | | | |
| nogRNA-G1322spilt-KKH- | C | 0.01% | 0.00% | 99.95% | 99.96% | 99.95% | 99.93% | 0.01% | 99.96% |
| Cas9-G1322-C and | | | | | | | | | |
| dSpCas9-G1322-N | | | | | | | | | |
| nogRNA-G1322spilt-KKH- | G | 0.02% | 99.98% | 0.01% | 0.01% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.01% | 0.01% | 0.01% | 0.01% | 0.01% | 0.03% | 0.01% | 0.01% |
| Cas9-G1322-C and | | | | | | | | | |
| dSpCas9-G1322-N | | | | | | | | | |
| nogRNA-G1333spilt-KKH- | A | 0.02% | 0.01% | 0.00% | 0.00% | 0.04% | 0.00% | 0.03% | 0.00% |
| Cas9-G1333-N and | | | | | | | | | |
| dSpCas9-G1333-C | | | | | | | | | |
| nogRNA-G1333spilt-KKH- | T | 99.95% | 0.00% | 0.01% | 0.02% | 0.02% | 0.00% | 99.95% | 0.03% |
| Cas9-G1333-N and | | | | | | | | | |
| dSpCas9-G1333-C | | | | | | | | | |
| nogRNA-G1333spilt-KKH- | C | 0.02% | 0.00% | 99.98% | 99.97% | 99.94% | 99.97% | 0.00% | 99.96% |
| Cas9-G1333-N and | | | | | | | | | |
| dSpCas9-G1333-C | | | | | | | | | |
| nogRNA-G1333spilt-KKH- | G | 0.01% | 99.99% | 0.00% | 0.00% | 0.00% | 0.00% | 0.01% | 0.00% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.00% | 0.00% | 0.00% | 0.00% | 0.00% | 0.02% | 0.00% | 0.00% |
| Cas9-G1333-N and | | | | | | | | | |
| dSpCas9-G1333-C | | | | | | | | | |
| nogRNA-G1333spilt-KKH- | A | 0.00% | 0.00% | 0.00% | 0.00% | 0.00% | 0.00% | 0.01% | 0.00% |
| Cas9-G1333-C and | | | | | | | | | |
| dSpCas9-G1333-N | | | | | | | | | |
| nogRNA-G1333spilt-KKH- | T | 99.98% | 0.03% | 0.00% | 0.01% | 0.01% | 0.01% | 99.97% | 0.02% |
| Cas9-G1333-C and | | | | | | | | | |
| dSpCas9-G1333-N | | | | | | | | | |
| nogRNA-G1333spilt-KKH- | C | 0.00% | 0.00% | 99.99% | 99.98% | 99.99% | 99.99% | 0.01% | 99.97% |
| Cas9-G1333-C and | | | | | | | | | |
| dSpCas9-G1333-N | | | | | | | | | |
| nogRNA-G1333spilt-KKH- | G | 0.01% | 99.97% | 0.00% | 0.00% | 0.00% | 0.00% | 0.01% | 0.00% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1371spilt-KKH- | A | 0.02% | 0.02% | 0.00% | 0.00% | 0.00% | 0.00% | 0.02% | 0.01% |
| Cas9-G1371-N and | | | | | | | | | |
| dSpCas9-G1371-C | | | | | | | | | |
| nogRNA-G1371spilt-KKH- | T | 99.92% | 0.01% | 0.02% | 0.00% | 0.00% | 0.02% | 99.97% | 0.01% |
| Cas9-G1371-N and | | | | | | | | | |
| dSpCas9-G1371-C | | | | | | | | | |
| nogRNA-G1371spilt-KKH- | C | 0.04% | 0.00% | 99% | 99.99% | 99.99% | 99.97% | 0.01% | 99.98% |
| Cas9-G1371-N and | | | | | | | | | |
| dSpCas9-G1371-C | | | | | | | | | |
| nogRNA-G1371spilt-KKH- | G | 0.01% | 99.96% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.00% | 0.00% | 0.00% | 0.00% | 0.00% | 0.01% | 0.00% | 0.00% |
| Cas9-G1371-N and | | | | | | | | | |
| dSpCas9-G1371-C | | | | | | | | | |
| nogRNA-G1371spilt-KKH- | A | 0.01% | 0.01% | 0.00% | 0.01% | 0.01% | 0.01% | 0.01% | 0.00% |
| Cas9-G1371-C and | | | | | | | | | |
| dSpCas9-G1371-N | | | | | | | | | |
| nogRNA-G1371spilt-KKH- | T | 99.92% | 0.01% | 0.01% | 0.02% | 0.01% | 0.01% | 99.92% | 0.01% |
| Cas9-G1371-C and | | | | | | | | | |
| dSpCas9-G1371-N | | | | | | | | | |
| nogRNA-G1371spilt-KKH- | C | 0.02% | 0.00% | 99.96% | 99.94% | 99.95% | 99.94% | 0.04% | 99.95% |
| Cas9-G1371-C and | | | | | | | | | |
| dSpCas9-G1371-N | | | | | | | | | |
| nogRNA-G1371spilt-KKH- | G | 0.01% | 99.94% | 0.00% | 0.00% | 0.00% | 0.00% | 0.01% | 0.00% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1371spilt-KKH- | — | 0.03% | 0.03% | 0.03% | 0.03% | 0.03% | 0.04% | 0.03% | 0.02% |
| Cas9-G1371-C and | | | | | | | | | |
| dSpCas9-G1371-N | | | | | | | | | |
| nogRNA-G1397spilt-KKH- | A | 0.00% | 0.02% | 0.00% | 0.01% | 0.02% | 0.01% | 0.00% | 0.02% |
| Cas9-G1397-N and | | | | | | | | | |
| dSpCas9-G1397-C | | | | | | | | | |
| nogRNA-G1397spilt-KKH- | T | 99.62% | 0.00% | 0.01% | 0.01% | 0.01% | 0.01% | 99.63% | 0.00% |
| Cas9-G1397-N and | | | | | | | | | |
| dSpCas9-G1397-C | | | | | | | | | |
| nogRNA-G1397spilt-KKH- | C | 0.02% | 0.01% | 99.65% | 99.64% | 99.64% | 99.64% | 0.02% | 99.64% |
| Cas9-G1397-N and | | | | | | | | | |
| dSpCas9-G1397-C | | | | | | | | | |
| nogRNA-G1397spilt-KKH- | G | 0.02% | 99.63% | 0.00% | 0.00% | 0.00% | 0.00% | 0.01% | 0.00% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.33% | 0.33% | 0.33% | 0.33% | 0.33% | 0.34% | 0.34% | 0.34% |
| Cas9-G1397-N and | | | | | | | | | |
| dSpCas9-G1397-C | | | | | | | | | |
| nogRNA-G1397spilt-KKH- | A | 0.01% | 0.02% | 0.00% | 0.01% | 0.02% | 0.01% | 0.02% | 0.01% |
| Cas9-G1397-C and | | | | | | | | | |
| dSpCas9-G1397-N | | | | | | | | | |
| nogRNA-G1397spilt-KKH- | T | 99.95% | 0.00% | 0.01% | 0.00% | 0.01% | 0.01% | 99.93% | 0.07% |
| Cas9-G1397-C and | | | | | | | | | |
| dSpCas9-G1397-N | | | | | | | | | |
| nogRNA-G1397spilt-KKH- | C | 0.01% | 0.01% | 99.96% | 99.97% | 99.95% | 99.93% | 0.02% | 99.89% |
| Cas9-G1397-C and | | | | | | | | | |
| dSpCas9-G1397-N | | | | | | | | | |
| nogRNA-G1397spilt-KKH- | G | 0.01% | 99.95% | 0.00% | 0.00% | 0.00% | 0.00% | 0.01% | 0.00% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.02% | 0.02% | 0.02% | 0.02% | 0.02% | 0.04% | 0.02% | 0.02% |
| Cas9-G1397-C and | | | | | | | | | |
| dSpCas9-G1397-N | | | | | | | | | |
| nogRNA-N1357spilt-KKH- | A | 0.01% | 0.03% | 0.01% | 0.01% | 0.00% | 0.01% | 0.01% | 0.00% |
| Cas9-N1357-N and | | | | | | | | | |
| dSpCas9-N1357-C | | | | | | | | | |
| nogRNA-N1357spilt-KKH- | T | 99.96% | 0.01% | 0.02% | 0.00% | 0.00% | 0.01% | 99.97% | 0.02% |
| Cas9-N1357-N and | | | | | | | | | |
| dSpCas9-N1357-C | | | | | | | | | |
| nogRNA-N1357spilt-KKH- | C | 0.02% | 0.00% | 99.97% | 99.98% | 99.99% | 99.97% | 0.01% | 99.97% |
| Cas9-N1357-N and | | | | | | | | | |
| dSpCas9-N1357-C | | | | | | | | | |
| nogRNA-N1357spilt-KKH- | G | 0.02% | 99.96% | 0.00% | 0.00% | 0.01% | 0.00% | 0.00% | 0.00% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.00% | 0.00% | 0.00% | 0.00% | 0.00% | 0.01% | 0.01% | 0.00% |
| Cas9-N1357-N and | | | | | | | | | |
| dSpCas9-N1357-C | | | | | | | | | |
| nogRNA-N1357spilt-KKH- | A | 0.01% | 0.02% | 0.00% | 0.02% | 0.01% | 0.00% | 0.02% | 0.01% |
| Cas9-N1357-C and | | | | | | | | | |
| dSpCas9-N1357-N | | | | | | | | | |
| nogRNA-N1357spilt-KKH- | T | 99.97% | 0.00% | 0.01% | 0.01% | 0.01% | 0.01% | 99.93% | 0.03% |
| Cas9-N1357-C and | | | | | | | | | |
| dSpCas9-N1357-N | | | | | | | | | |
| nogRNA-N1357spilt-KKH- | C | 0.02% | 0.01% | 99.99% | 99.97% | 99.99% | 99.98% | 0.04% | 99.97% |
| Cas9-N1357-C and | | | | | | | | | |
| dSpCas9-N1357-N | | | | | | | | | |
| nogRNA-N1357spilt-KKH- | G | 0.01% | 99.97% | 0.00% | 0.00% | 0.00% | 0.00% | 0.01% | 0.00% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.00% | 0.00% | 0.00% | 0.00% | 0.00% | 0.01% | 0.00% | 0.00% |
| Cas9-N1357-C and | | | | | | | | | |
| dSpCas9-N1357-N | | | | | | | | | |
| nogRNA-N1387spilt-KKH- | A | 0.02% | 0.02% | 0.01% | 0.00% | 0.02% | 0.02% | 0.01% | 0.01% |
| Cas9-N1387-N and | | | | | | | | | |
| dSpCas9-N1387-C | | | | | | | | | |
| nogRNA-N1387spilt-KKH- | T | 99.92% | 0.00% | 0.00% | 0.03% | 0.01% | 0.01% | 99.93% | 0.03% |
| Cas9-N1387-N and | | | | | | | | | |
| dSpCas9-N1387-C | | | | | | | | | |
| nogRNA-N1387spilt-KKH- | C | 0.03% | 0.01% | 99.96% | 99.94% | 99.95% | 99.92% | 0.03% | 99.93% |
| Cas9-N1387-N and | | | | | | | | | |
| dSpCas9-N1387-C | | | | | | | | | |
| nogRNA-N1387spilt-KKH- | G | 0.01% | 99.94% | 0.00% | 0.01% | 0.00% | 0.02% | 0.00% | 0.00% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.02% | 0.02% | 0.02% | 0.02% | 0.02% | 0.04% | 0.02% | 0.02% |
| Cas9-N1387-N and | | | | | | | | | |
| dSpCas9-N1387-C | | | | | | | | | |
| nogRNA-N1387spilt-KKH- | A | 0.01% | 0.04% | 0.01% | 0.01% | 0.02% | 0.01% | 0.01% | 0.00% |
| Cas9-N1387-C and | | | | | | | | | |
| dSpCas9-N1387-N | | | | | | | | | |
| nogRNA-N1387spilt-KKH- | T | 99.95% | 0.01% | 0.01% | 0.01% | 0.01% | 0.00% | 99.96% | 0.04% |
| Cas9-N1387-C and | | | | | | | | | |
| dSpCas9-N1387-N | | | | | | | | | |
| nogRNA-N1387spilt-KKH- | C | 0.01% | 0.00% | 99.96% | 99.96% | 99.96% | 99.96% | 0.00% | 99.95% |
| Cas9-N1387-C and | | | | | | | | | |
| dSpCas9-N1387-N | | | | | | | | | |
| nogRNA-N1387spilt-KKH- | G | 0.02% | 99.94% | 0.01% | 0.00% | 0.00% | 0.00% | 0.01% | 0.00% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.01% | 0.01% | 0.01% | 0.01% | 0.01% | 0.02% | 0.01% | 0.01% |
| Cas9-N1387-C and | | | | | | | | | |
| dSpCas9-N1387-N | | | | | | | | | |

| Batch | Nucleotide | C | C | T | C | C | C | T |
|---|---|---|---|---|---|---|---|---|
| nogRNA-A1343spilt-KKH- | A | 0.00% | 0.01% | 0.00% | 0.01% | 0.01% | 0.02% | 0.01% |
| Cas9-A1343-N and | | | | | | | | |
| dSpCas9-A1343-C | | | | | | | | |
| nogRNA-A1343spilt-KKH- | T | 0.01% | 0.02% | 99.97% | 0.02% | 0.00% | 0.01% | 99.94% |
| Cas9-A1343-N and | | | | | | | | |
| dSpCas9-A1343-C | | | | | | | | |
| nogRNA-A1343spilt-KKH- | C | 99.98% | 99.95% | 0.02% | 99.96% | 99.98% | 99.96% | 0.02% |
| Cas9-A1343-N and | | | | | | | | |
| dSpCas9-A1343-C | | | | | | | | |
| nogRNA-A1343spilt-KKH- | G | 0.00% | 0.00% | 0.00% | 0.00% | 0.00% | 0.00% | 0.02% |
| Cas9-A1343-N and | | | | | | | | |
| dSpCas9-A1343-C | | | | | | | | |
| nogRNA-A1343spilt-KKH- | N | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | — | 0.00% | 0.01% | 0.00% | 0.00% | 0.00% | 0.01% | 0.01% |
| Cas9-A1343-N and | | | | | | | | |
| dSpCas9-A1343-C | | | | | | | | |
| nogRNA-A1343spilt-KKH- | A | 0.01% | 0.03% | 0.02% | 0.01% | 0.00% | 0.01% | 0.01% |
| Cas9-A1343-C and | | | | | | | | |
| dSpCas9-A1343-N | | | | | | | | |
| nogRNA-A1343spilt-KKH- | T | 0.01% | 0.01% | 99.95% | 0.01% | 0.02% | 0.00% | 99.92% |
| Cas9-A1343-C and | | | | | | | | |
| dSpCas9-A1343-N | | | | | | | | |
| nogRNA-A1343spilt-KKH- | C | 99.98% | 99.95% | 0.03% | 99.97% | 99.96% | 99.97% | 0.04% |
| Cas9-A1343-C and | | | | | | | | |
| dSpCas9-A1343-N | | | | | | | | |
| nogRNA-A1343spilt-KKH- | G | 0.00% | 0.00% | 0.00% | 0.00% | 0.00% | 0.00% | 0.02% |
| Cas9-A1343-C and | | | | | | | | |
| dSpCas9-A1343-N | | | | | | | | |
| nogRNA-A1343spilt-KKH- | N | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-A1343spilt-KKH- | — | 0.00% | 0.00% | 0.00% | 0.00% | 0.01% | 0.02% | 0.01% |
| Cas9-A1343-C and | | | | | | | | |
| dSpCas9-A1343-N | | | | | | | | |
| nogRNA-G1322spilt-KKH- | A | 0.02% | 0.01% | 0.01% | 0.01% | 0.01% | 0.02% | 0.02% |
| Cas9-G1322-N and | | | | | | | | |
| dSpCas9-G1322-C | | | | | | | | |
| nogRNA-G1322spilt-KKH- | T | 0.03% | 0.01% | 99.97% | 0.02% | 0.02% | 0.01% | 99.92% |
| Cas9-G1322-N and | | | | | | | | |
| dSpCas9-G1322-C | | | | | | | | |
| nogRNA-G1322spilt-KKH- | C | 99.95% | 99.97% | 0.02% | 99.96% | 99.96% | 99.95% | 0.04% |
| Cas9-G1322-N and | | | | | | | | |
| dSpCas9-G1322-C | | | | | | | | |
| nogRNA-G1322spilt-KKH- | G | 0.00% | 0.00% | 0.01% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | N | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | — | 0.00% | 0.01% | 0.00% | 0.01% | 0.01% | 0.02% | 0.02% |
| Cas9-G1322-N and | | | | | | | | |
| dSpCas9-G1322-C | | | | | | | | |
| nogRNA-G1322spilt-KKH- | A | 0.00% | 0.01% | 0.05% | 0.02% | 0.01% | 0.02% | 0.01% |
| Cas9-G1322-C and | | | | | | | | |
| dSpCas9-G1322-N | | | | | | | | |
| nogRNA-G1322spilt-KKH- | T | 0.01% | 0.01% | 99.93% | 0.10% | 0.00% | 0.02% | 99.92% |
| Cas9-G1322-C and | | | | | | | | |
| dSpCas9-G1322-N | | | | | | | | |
| nogRNA-G1322spilt-KKH- | C | 99.98% | 99.96% | 0.02% | 99.87% | 99.97% | 99.95% | 0.04% |
| Cas9-G1322-C and | | | | | | | | |
| dSpCas9-G1322-N | | | | | | | | |
| nogRNA-G1322spilt-KKH- | G | 0.00% | 0.00% | 0.00% | 0.00% | 0.00% | 0.00% | 0.02% |
| Cas9-G1322-C and | | | | | | | | |
| dSpCas9-G1322-N | | | | | | | | |
| nogRNA-G1322spilt-KKH- | N | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1322spilt-KKH- | — | 0.01% | 0.02% | 0.01% | 0.01% | 0.00% | 0.01% | 0.01% |
| Cas9-G1322-C and | | | | | | | | |
| dSpCas9-G1322-N | | | | | | | | |
| nogRNA-G1333spilt-KKH- | A | 0.02% | 0.01% | 0.01% | 0.01% | 0.00% | 0.03% | 0.00% |
| Cas9-G1333-N and | | | | | | | | |
| dSpCas9-G1333-C | | | | | | | | |
| nogRNA-G1333spilt-KKH- | T | 0.03% | 0.01% | 99.96% | 0.03% | 0.02% | 0.01% | 99.91% |
| Cas9-G1333-N and | | | | | | | | |
| dSpCas9-G1333-C | | | | | | | | |
| nogRNA-G1333spilt-KKH- | C | 99.95% | 99.97% | 0.02% | 99.95% | 99.97% | 99.94% | 0.04% |
| Cas9-G1333-N and | | | | | | | | |
| dSpCas9-G1333-C | | | | | | | | |
| nogRNA-G1333spilt-KKH- | G | 0.00% | 0.00% | 0.00% | 0.00% | 0.00% | 0.00% | 0.04% |
| Cas9-G1333-N and | | | | | | | | |
| dSpCas9-G1333-C | | | | | | | | |
| nogRNA-G1333spilt-KKH- | N | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | — | 0.00% | 0.01% | 0.01% | 0.01% | 0.01% | 0.01% | 0.01% |
| Cas9-G1333-N and | | | | | | | | |
| dSpCas9-G1333-C | | | | | | | | |
| nogRNA-G1333spilt-KKH- | A | 0.00% | 0.01% | 0.01% | 0.01% | 0.02% | 0.00% | 0.01% |
| Cas9-G1333-C and | | | | | | | | |
| dSpCas9-G1333-N | | | | | | | | |
| nogRNA-G1333spilt-KKH- | T | 0.01% | 0.01% | 99.96% | 0.03% | 0.01% | 0.01% | 99.90% |
| Cas9-G1333-C and | | | | | | | | |
| dSpCas9-G1333-N | | | | | | | | |
| nogRNA-G1333spilt-KKH- | C | 99.99% | 99.96% | 0.01% | 99.91% | 99.90% | 99.93% | 0.04% |
| Cas9-G1333-C and | | | | | | | | |
| dSpCas9-G1333-N | | | | | | | | |
| nogRNA-G1333spilt-KKH- | G | 0.00% | 0.00% | 0.01% | 0.00% | 0.01% | 0.00% | 0.00% |
| Cas9-G1333-C and | | | | | | | | |
| dSpCas9-G1333-N | | | | | | | | |
| nogRNA-G1333spilt-KKH- | N | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1333spilt-KKH- | — | 0.00% | 0.02% | 0.00% | 0.05% | 0.05% | 0.05% | 0.05% |
| Cas9-G1333-C and | | | | | | | | |
| dSpCas9-G1333-N | | | | | | | | |
| nogRNA-G1371spilt-KKH- | A | 0.00% | 0.02% | 0.01% | 0.00% | 0.01% | 0.02% | 0.00% |
| Cas9-G1371-N and | | | | | | | | |
| dSpCas9-G1371-C | | | | | | | | |
| nogRNA-G1371spilt-KKH- | T | 0.02% | 0.01% | 99.96% | 0.02% | 0.01% | 0.00% | 99.92% |
| Cas9-G1371-N and | | | | | | | | |
| dSpCas9-G1371-C | | | | | | | | |
| nogRNA-G1371spilt-KKH- | C | 99.97% | 99.95% | 0.02% | 99.95% | 99.94% | 99.94% | 0.02% |
| Cas9-G1371-N and | | | | | | | | |
| dSpCas9-G1371-C | | | | | | | | |
| nogRNA-G1371spilt-KKH- | G | 0.00% | 0.00% | 0.00% | 0.00% | 0.00% | 0.00% | 0.02% |
| Cas9-G1371-N and | | | | | | | | |
| dSpCas9-G1371-C | | | | | | | | |
| nogRNA-G1371spilt-KKH- | N | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | — | 0.00% | 0.01% | 0.01% | 0.03% | 0.03% | 0.04% | 0.04% |
| Cas9-G1371-N and | | | | | | | | |
| dSpCas9-G1371-C | | | | | | | | |
| nogRNA-G1371spilt-KKH- | A | 0.00% | 0.02% | 0.02% | 0.01% | 0.00% | 0.01% | 0.01% |
| Cas9-G1371-C and | | | | | | | | |
| dSpCas9-G1371-N | | | | | | | | |
| nogRNA-G1371spilt-KKH- | T | 0.02% | 0.01% | 99.92% | 0.01% | 0.02% | 0.01% | 99.93% |
| Cas9-G1371-C and | | | | | | | | |
| dSpCas9-G1371-N | | | | | | | | |
| nogRNA-G1371spilt-KKH- | C | 99.95% | 99.95% | 0.03% | 99.96% | 99.95% | 99.95% | 0.03% |
| Cas9-G1371-C and | | | | | | | | |
| dSpCas9-G1371-N | | | | | | | | |
| nogRNA-G1371spilt-KKH- | G | 0.00% | 0.00% | 0.01% | 0.00% | 0.00% | 0.00% | 0.01% |
| Cas9-G1371-C and | | | | | | | | |
| dSpCas9-G1371-N | | | | | | | | |
| nogRNA-G1371spilt-KKH- | N | 0.00% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1371spilt-KKH- | — | 0.02% | 0.02% | 0.02% | 0.02% | 0.02% | 0.02% | 0.02% |
| Cas9-G1371-C and | | | | | | | | |
| dSpCas9-G1371-N | | | | | | | | |
| nogRNA-G1397spilt-KKH- | A | 0.00% | 0.01% | 0.01% | 0.01% | 0.02% | 0.00% | 0.01% |
| Cas9-G1397-N and | | | | | | | | |
| dSpCas9-G1397-C | | | | | | | | |
| nogRNA-G1397spilt-KKH- | T | 0.01% | 0.00% | 99.62% | 0.02% | 0.00% | 0.00% | 99.58% |
| Cas9-G1397-N and | | | | | | | | |
| dSpCas9-G1397-C | | | | | | | | |
| nogRNA-G1397spilt-KKH- | C | 99.65% | 99.66% | 0.02% | 99.61% | 99.62% | 99.64% | 0.02% |
| Cas9-G1397-N and | | | | | | | | |
| dSpCas9-G1397-C | | | | | | | | |
| nogRNA-G1397spilt-KKH- | G | 0.00% | 0.00% | 0.01% | 0.00% | 0.00% | 0.00% | 0.02% |
| Cas9-G1397-N and | | | | | | | | |
| dSpCas9-G1397-C | | | | | | | | |
| nogRNA-G1397spilt-KKH- | N | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | — | 0.34% | 0.34% | 0.34% | 0.36% | 0.36% | 0.36% | 0.36% |
| Cas9-G1397-N and | | | | | | | | |
| dSpCas9-G1397-C | | | | | | | | |
| nogRNA-G1397spilt-KKH- | A | 0.02% | 0.01% | 0.01% | 0.01% | 0.00% | 0.01% | 0.01% |
| Cas9-G1397-C and | | | | | | | | |
| dSpCas9-G1397-N | | | | | | | | |
| nogRNA-G1397spilt-KKH- | T | 0.04% | 0.03% | 99.93% | 0.06% | 0.05% | 0.04% | 99.91% |
| Cas9-G1397-C and | | | | | | | | |
| dSpCas9-G1397-N | | | | | | | | |
| nogRNA-G1397spilt-KKH- | C | 99.91% | 99.93% | 0.03% | 99.90% | 99.93% | 99.92% | 0.04% |
| Cas9-G1397-C and | | | | | | | | |
| dSpCas9-G1397-N | | | | | | | | |
| nogRNA-G1397spilt-KKH- | G | 0.00% | 0.00% | 0.00% | 0.00% | 0.00% | 0.00% | 0.01% |
| Cas9-G1397-C and | | | | | | | | |
| dSpCas9-G1397-N | | | | | | | | |
| nogRNA-G1397spilt-KKH- | N | 0.00% | 0.00% | 0.00% | 0.00% | 0.00% | 0.00% | 0.00% |
| Cas9-G1397-C and | | | | | | | | |
| dSpCas9-G1397-N | | | | | | | | |
| nogRNA-G1397spilt-KKH- | — | 0.02% | 0.02% | 0.02% | 0.03% | 0.02% | 0.03% | 0.03% |
| Cas9-G1397-C and | | | | | | | | |
| dSpCas9-G1397-N | | | | | | | | |
| nogRNA-N1357spilt-KKH- | A | 0.01% | 0.01% | 0.00% | 0.00% | 0.02% | 0.03% | 0.01% |
| Cas9-N1357-N and | | | | | | | | |
| dSpCas9-N1357-C | | | | | | | | |
| nogRNA-N1357spilt-KKH- | T | 0.03% | 0.01% | 99.99% | 0.02% | 0.03% | 0.00% | 99.91% |
| Cas9-N1357-N and | | | | | | | | |
| dSpCas9-N1357-C | | | | | | | | |
| nogRNA-N1357spilt-KKH- | C | 99.96% | 99.96% | 0.00% | 99.96% | 99.92% | 99.95% | 0.04% |
| Cas9-N1357-N and | | | | | | | | |
| dSpCas9-N1357-C | | | | | | | | |
| nogRNA-N1357spilt-KKH- | G | 0.00% | 0.01% | 0.00% | 0.00% | 0.01% | 0.00% | 0.02% |
| Cas9-N1357-N and | | | | | | | | |
| dSpCas9-N1357-C | | | | | | | | |
| nogRNA-N1357spilt-KKH- | N | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | — | 0.00% | 0.02% | 0.01% | 0.02% | 0.02% | 0.02% | 0.02% |
| Cas9-N1357-N and | | | | | | | | |
| dSpCas9-N1357-C | | | | | | | | |
| nogRNA-N1357spilt-KKH- | A | 0.00% | 0.02% | 0.01% | 0.01% | 0.01% | 0.03% | 0.01% |
| Cas9-N1357-C and | | | | | | | | |
| dSpCas9-N1357-N | | | | | | | | |
| nogRNA-N1357spilt-KKH- | T | 0.02% | 0.01% | 99.96% | 0.02% | 0.02% | 0.00% | 99.95% |
| Cas9-N1357-C and | | | | | | | | |
| dSpCas9-N1357-N | | | | | | | | |
| nogRNA-N1357spilt-KKH- | C | 99.97% | 99.96% | 0.03% | 99.97% | 99.97% | 99.94% | 0.03% |
| Cas9-N1357-C and | | | | | | | | |
| dSpCas9-N1357-N | | | | | | | | |
| nogRNA-N1357spilt-KKH- | G | 0.01% | 0.00% | 0.00% | 0.00% | 0.00% | 0.01% | 0.01% |
| Cas9-N1357-C and | | | | | | | | |
| dSpCas9-N1357-N | | | | | | | | |
| nogRNA-N1357spilt-KKH- | N | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1357spilt-KKH- | — | 0.00% | 0.01% | 0.01% | 0.01% | 0.01% | 0.02% | 0.01% |
| Cas9-N1357-C and | | | | | | | | |
| dSpCas9-N1357-N | | | | | | | | |
| nogRNA-N1387spilt-KKH- | A | 0.01% | 0.02% | 0.00% | 0.02% | 0.02% | 0.01% | 0.00% |
| Cas9-N1387-N and | | | | | | | | |
| dSpCas9-N1387-C | | | | | | | | |
| nogRNA-N1387spilt-KKH- | T | 0.02% | 0.01% | 99.93% | 0.06% | 0.05% | 0.03% | 99.92% |
| Cas9-N1387-N and | | | | | | | | |
| dSpCas9-N1387-C | | | | | | | | |
| nogRNA-N1387spilt-KKH- | C | 99.94% | 99.94% | 0.04% | 99.89% | 99.89% | 99.91% | 0.03% |
| Cas9-N1387-N and | | | | | | | | |
| dSpCas9-N1387-C | | | | | | | | |
| nogRNA-N1387spilt-KKH- | G | 0.00% | 0.00% | 0.00% | 0.00% | 0.01% | 0.00% | 0.02% |
| Cas9-N1387-N and | | | | | | | | |
| dSpCas9-N1387-C | | | | | | | | |
| nogRNA-N1387spilt-KKH- | N | 0.00% | 0.00% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | — | 0.02% | 0.03% | 0.02% | 0.03% | 0.04% | 0.04% | 0.04% |
| Cas9-N1387-N and | | | | | | | | |
| dSpCas9-N1387-C | | | | | | | | |
| nogRNA-N1387spilt-KKH- | A | 0.01% | 0.01% | 0.00% | 0.01% | 0.04% | 0.01% | 0.00% |
| Cas9-N1387-C and | | | | | | | | |
| dSpCas9-N1387-N | | | | | | | | |
| nogRNA-N1387spilt-KKH- | T | 0.04% | 0.04% | 99.95% | 0.02% | 0.04% | 0.02% | 99.93% |
| Cas9-N1387-C and | | | | | | | | |
| dSpCas9-N1387-N | | | | | | | | |
| nogRNA-N1387spilt-KKH- | C | 99.94% | 99.93% | 0.03% | 99.94% | 99.90% | 99.95% | 0.05% |
| Cas9-N1387-C and | | | | | | | | |
| dSpCas9-N1387-N | | | | | | | | |
| nogRNA-N1387spilt-KKH- | G | 0.00% | 0.00% | 0.00% | 0.01% | 0.00% | 0.00% | 0.01% |
| Cas9-N1387-C and | | | | | | | | |
| dSpCas9-N1387-N | | | | | | | | |
| nogRNA-N1387spilt-KKH- | N | 0.00% | 0.00% | 0.00% | 0.00% | 0.00% | 0.00% | 0.00% |
| Cas9-N1387-C and | | | | | | | | |
| dSpCas9-N1387-N | | | | | | | | |
| nogRNA-N1387spilt-KKH- | — | 0.01% | 0.02% | 0.02% | 0.02% | 0.02% | 0.01% | 0.01% |
| Cas9-N1387-C and | | | | | | | | |
| dSpCas9-N1387-N | | | | | | | | |

| Batch | Nucleotide | G | G | C | C | C | A | G | G | T |
|---|---|---|---|---|---|---|---|---|---|---|
| nogRNA-A1343spilt-KKH- | A | 0.01% | 0.00% | 0.01% | 0.01% | 0.01% | 99.93% | 0.00% | 0.01% | 0.00% |
| Cas9-A1343-N and | | | | | | | | | | |
| dSpCas9-A1343-C | | | | | | | | | | |
| nogRNA-A1343spilt-KKH- | T | 0.01% | 0.00% | 0.01% | 0.01% | 0.01% | 0.02% | 0.01% | 0.00% | 99.77% |
| Cas9-A1343-N and | | | | | | | | | | |
| dSpCas9-A1343-C | | | | | | | | | | |
| nogRNA-A1343spilt-KKH- | C | 0.00% | 0.00% | 99.98% | 99.98% | 99.98% | 0.00% | 0.00% | 0.00% | 0.05% |
| Cas9-A1343-N and | | | | | | | | | | |
| dSpCas9-A1343-C | | | | | | | | | | |
| nogRNA-A1343spilt-KKH- | G | 99.98% | 100.00% | 0.01% | 0.00% | 0.00% | 0.05% | 99.98% | 99.98% | 0.17% |
| Cas9-A1343-N and | | | | | | | | | | |
| dSpCas9-A1343-C | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | A | 0.02% | 0.00% | 0.00% | 0.00% | 0.01% | 99.84% | 0.00% | 0.01% | 0.00% |
| Cas9-A1343-C and | | | | | | | | | | |
| dSpCas9-A1343-N | | | | | | | | | | |
| nogRNA-A1343spilt-KKH- | T | 0.00% | 0.01% | 0.01% | 0.01% | 0.01% | 0.00% | 0.01% | 0.00% | 99.71% |
| Cas9-A1343-C and | | | | | | | | | | |
| dSpCas9-A1343-N | | | | | | | | | | |
| nogRNA-A1343spilt-KKH- | C | 0.00% | 0.00% | 99.99% | 99.98% | 99.97% | 0.08% | 0.00% | 0.00% | 0.04% |
| Cas9-A1343-C and | | | | | | | | | | |
| dSpCas9-A1343-N | | | | | | | | | | |
| nogRNA-A1343spilt-KKH- | G | 99.98% | 99.98% | 0.00% | 0.00% | 0.00% | 0.08% | 99.99% | 99.98% | 0.25% |
| Cas9-A1343-C and | | | | | | | | | | |
| dSpCas9-A1343-N | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-A1343spilt-KKH- | — | 0.00% | 0.00% | 0.00% | 0.00% | 0.01% | 0.00% | 0.00% | 0.00% | 0.00% |
| Cas9-A1343-C and | | | | | | | | | | |
| dSpCas9-A1343-N | | | | | | | | | | |
| nogRNA-G1322spilt-KKH- | A | 0.02% | 0.01% | 0.00% | 0.00% | 0.03% | 99.90% | 0.00% | 0.01% | 0.03% |
| Cas9-G1322-N and | | | | | | | | | | |
| dSpCas9-G1322-C | | | | | | | | | | |
| nogRNA-G1322spilt-KKH- | T | 0.00% | 0.00% | 0.02% | 0.01% | 0.02% | 0.02% | 0.01% | 0.00% | 99.74% |
| Cas9-G1322-N and | | | | | | | | | | |
| dSpCas9-G1322-C | | | | | | | | | | |
| nogRNA-G1322spilt-KKH- | C | 0.00% | 0.01% | 99.97% | 99.98% | 99.95% | 0.02% | 0.01% | 0.00% | 0.05% |
| Cas9-G1322-N and | | | | | | | | | | |
| dSpCas9-G1322-C | | | | | | | | | | |
| nogRNA-G1322spilt-KKH- | G | 99.98% | 99.98% | 0.01% | 0.01% | 0.00% | 0.06% | 99.98% | 99.99% | 0.19% |
| Cas9-G1322-N and | | | | | | | | | | |
| dSpCas9-G1322-C | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | A | 0.00% | 0.02% | 0.00% | 0.01% | 0.02% | 99.92% | 0.01% | 0.00% | 0.02% |
| Cas9-G1322-C and | | | | | | | | | | |
| dSpCas9-G1322-N | | | | | | | | | | |
| nogRNA-G1322spilt-KKH- | T | 0.01% | 0.00% | 0.01% | 0.01% | 0.02% | 0.01% | 0.01% | 0.00% | 99.71% |
| Cas9-G1322-C and | | | | | | | | | | |
| dSpCas9-G1322-N | | | | | | | | | | |
| nogRNA-G1322spilt-KKH- | C | 0.00% | 0.00% | 99.96% | 99.97% | 99.95% | 0.01% | 0.00% | 0.00% | 0.05% |
| Cas9-G1322-C and | | | | | | | | | | |
| dSpCas9-G1322-N | | | | | | | | | | |
| nogRNA-G1322spilt-KKH- | G | 99.98% | 99.97% | 0.02% | 0.00% | 0.00% | 0.05% | 99.97% | 99.99% | 0.22% |
| Cas9-G1322-C and | | | | | | | | | | |
| dSpCas9-G1322-N | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1322spilt-KKH- | — | 0.00% | 0.01% | 0.00% | 0.00% | 0.01% | 0.00% | 0.00% | 0.00% | 0.00% |
| Cas9-G1322-C and | | | | | | | | | | |
| dSpCas9-G1322-N | | | | | | | | | | |
| nogRNA-G1333spilt-KKH- | A | 0.03% | 0.03% | 0.00% | 0.00% | 0.00% | 99.91% | 0.00% | 0.00% | 0.02% |
| Cas9-G1333-N and | | | | | | | | | | |
| dSpCas9-G1333-C | | | | | | | | | | |
| nogRNA-G1333spilt-KKH- | T | 0.00% | 0.00% | 0.01% | 0.02% | 0.03% | 0.02% | 0.02% | 0.00% | 99.70% |
| Cas9-G1333-N and | | | | | | | | | | |
| dSpCas9-G1333-C | | | | | | | | | | |
| nogRNA-G1333spilt-KKH- | C | 0.00% | 0.00% | 99.97% | 99.96% | 99.96% | 0.03% | 0.00% | 0.00% | 0.06% |
| Cas9-G1333-N and | | | | | | | | | | |
| dSpCas9-G1333-C | | | | | | | | | | |
| nogRNA-G1333spilt-KKH- | G | 99.97% | 99.96% | 0.02% | 0.01% | 0.00% | 0.04% | 99.98% | 99.99% | 0.21% |
| Cas9-G1333-N and | | | | | | | | | | |
| dSpCas9-G1333-C | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | — | 0.00% | 0.01% | 0.00% | 0.00% | 0.01% | 0.00% | 0.00% | 0.00% | 0.01% |
| Cas9-G1333-N and | | | | | | | | | | |
| dSpCas9-G1333-C | | | | | | | | | | |
| nogRNA-G1333spilt-KKH- | A | 0.01% | 0.00% | 0.01% | 0.00% | 0.00% | 99.88% | 0.00% | 0.00% | 0.01% |
| Cas9-G1333-C and | | | | | | | | | | |
| dSpCas9-G1333-N | | | | | | | | | | |
| nogRNA-G1333spilt-KKH- | T | 0.00% | 0.01% | 0.02% | 0.00% | 0.03% | 0.01% | 0.01% | 0.01% | 99.69% |
| Cas9-G1333-C and | | | | | | | | | | |
| dSpCas9-G1333-N | | | | | | | | | | |
| nogRNA-G1333spilt-KKH- | C | 0.00% | 0.00% | 99.95% | 99.99% | 99.95% | 0.02% | 0.00% | 0.00% | 0.07% |
| Cas9-G1333-C and | | | | | | | | | | |
| dSpCas9-G1333-N | | | | | | | | | | |
| nogRNA-G1333spilt-KKH- | G | 99.99% | 99.98% | 0.02% | 0.00% | 0.01% | 0.09% | 99.99% | 99.99% | 0.22% |
| Cas9-G1333-C and | | | | | | | | | | |
| dSpCas9-G1333-N | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1371spilt-KKH- | A | 0.02% | 0.00% | 0.00% | 0.00% | 0.02% | 99.91% | 0.00% | 0.01% | 0.02% |
| Cas9-G1371-N and | | | | | | | | | | |
| dSpCas9-G1371-C | | | | | | | | | | |
| nogRNA-G1371spilt-KKH- | T | 0.00% | 0.00% | 0.04% | 0.02% | 0.02% | 0.02% | 0.01% | 0.00% | 99.71% |
| Cas9-G1371-N and | | | | | | | | | | |
| dSpCas9-G1371-C | | | | | | | | | | |
| nogRNA-G1371spilt-KKH- | C | 0.00% | 0.00% | 99.94% | 99.97% | 99.95% | 0.01% | 0.00% | 0.00% | 0.08% |
| Cas9-G1371-N and | | | | | | | | | | |
| dSpCas9-G1371-C | | | | | | | | | | |
| nogRNA-G1371spilt-KKH- | G | 99.97% | 99.98% | 0.01% | 0.00% | 0.00% | 0.06% | 99.98% | 99.98% | 0.19% |
| Cas9-G1371-N and | | | | | | | | | | |
| dSpCas9-G1371-C | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | — | 0.00% | 0.01% | 0.01% | 0.01% | 0.02% | 0.01% | 0.01% | 0.00% | 0.00% |
| Cas9-G1371-N and | | | | | | | | | | |
| dSpCas9-G1371-C | | | | | | | | | | |
| nogRNA-G1371spilt-KKH- | A | 0.01% | 0.01% | 0.00% | 0.01% | 0.01% | 99.89% | 0.00% | 0.00% | 0.01% |
| Cas9-G1371-C and | | | | | | | | | | |
| dSpCas9-G1371-N | | | | | | | | | | |
| nogRNA-G1371spilt-KKH- | T | 0.00% | 0.01% | 0.03% | 0.02% | 0.02% | 0.02% | 0.02% | 0.00% | 99.73% |
| Cas9-G1371-C and | | | | | | | | | | |
| dSpCas9-G1371-N | | | | | | | | | | |
| nogRNA-G1371spilt-KKH- | C | 0.00% | 0.01% | 99.96% | 99.96% | 99.95% | 0.03% | 0.00% | 0.00% | 0.06% |
| Cas9-G1371-C and | | | | | | | | | | |
| dSpCas9-G1371-N | | | | | | | | | | |
| nogRNA-G1371spilt-KKH- | G | 99.98% | 99.96% | 0.00% | 0.01% | 0.00% | 0.06% | 99.98% | 99.99% | 0.20% |
| Cas9-G1371-C and | | | | | | | | | | |
| dSpCas9-G1371-N | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1371spilt-KKH- | — | 0.01% | 0.01% | 0.00% | 0.00% | 0.01% | 0.00% | 0.00% | 0.00% | 0.01% |
| Cas9-G1371-C and | | | | | | | | | | |
| dSpCas9-G1371-N | | | | | | | | | | |
| nogRNA-G1397spilt-KKH- | A | 0.00% | 0.02% | 0.00% | 0.01% | 0.02% | 99.62% | 0.02% | 0.01% | 0.01% |
| Cas9-G1397-N and | | | | | | | | | | |
| dSpCas9-G1397-C | | | | | | | | | | |
| nogRNA-G1397spilt-KKH- | T | 0.00% | 0.00% | 0.05% | 0.03% | 0.01% | 0.02% | 0.00% | 0.00% | 99.53% |
| Cas9-G1397-N and | | | | | | | | | | |
| dSpCas9-G1397-C | | | | | | | | | | |
| nogRNA-G1397spilt-KKH- | C | 0.00% | 0.00% | 99.61% | 99.64% | 99.65% | 0.01% | 0.00% | 0.00% | 0.09% |
| Cas9-G1397-N and | | | | | | | | | | |
| dSpCas9-G1397-C | | | | | | | | | | |
| nogRNA-G1397spilt-KKH- | G | 99.66% | 99.65% | 0.01% | 0.00% | 0.00% | 0.04% | 99.66% | 99.81% | 0.19% |
| Cas9-G1397-N and | | | | | | | | | | |
| dSpCas9-G1397-C | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | — | 0.33% | 0.33% | 0.32% | 0.32% | 0.32% | 0.31% | 0.31% | 0.18% | 0.18% |
| Cas9-G1397-N and | | | | | | | | | | |
| dSpCas9-G1397-C | | | | | | | | | | |
| nogRNA-G1397spilt-KKH- | A | 0.02% | 0.02% | 0.00% | 0.00% | 0.01% | 99.90% | 0.00% | 0.02% | 0.01% |
| Cas9-G1397-C and | | | | | | | | | | |
| dSpCas9-G1397-N | | | | | | | | | | |
| nogRNA-G1397spilt-KKH- | T | 0.00% | 0.00% | 0.04% | 0.02% | 0.01% | 0.00% | 0.01% | 0.00% | 99.71% |
| Cas9-G1397-C and | | | | | | | | | | |
| dSpCas9-G1397-N | | | | | | | | | | |
| nogRNA-G1397spilt-KKH- | C | 0.00% | 0.00% | 99.94% | 99.96% | 99.97% | 0.01% | 0.00% | 0.00% | 0.06% |
| Cas9-G1397-C and | | | | | | | | | | |
| dSpCas9-G1397-N | | | | | | | | | | |
| nogRNA-G1397spilt-KKH- | G | 99.95% | 99.96% | 0.01% | 0.01% | 0.00% | 0.07% | 99.97% | 99.97% | 0.20% |
| Cas9-G1397-C and | | | | | | | | | | |
| dSpCas9-G1397-N | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-G1397spilt-KKH- | — | 0.02% | 0.01% | 0.01% | 0.01% | 0.02% | 0.02% | 0.01% | 0.01% | 0.01% |
| Cas9-G1397-C and | | | | | | | | | | |
| dSpCas9-G1397-N | | | | | | | | | | |
| nogRNA-N1357spilt-KKH- | A | 0.01% | 0.01% | 0.00% | 0.00% | 0.01% | 99.91% | 0.01% | 0.02% | 0.03% |
| Cas9-N1357-N and | | | | | | | | | | |
| dSpCas9-N1357-C | | | | | | | | | | |
| nogRNA-N1357spilt-KKH- | T | 0.01% | 0.02% | 0.02% | 0.02% | 0.02% | 0.01% | 0.00% | 0.00% | 99.71% |
| Cas9-N1357-N and | | | | | | | | | | |
| dSpCas9-N1357-C | | | | | | | | | | |
| nogRNA-N1357spilt-KKH- | C | 0.00% | 0.00% | 99.95% | 99.97% | 99.97% | 0.02% | 0.00% | 0.00% | 0.08% |
| Cas9-N1357-N and | | | | | | | | | | |
| dSpCas9-N1357-C | | | | | | | | | | |
| nogRNA-N1357spilt-KKH- | G | 99.98% | 99.97% | 0.03% | 0.00% | 0.00% | 0.05% | 99.99% | 99.98% | 0.18% |
| Cas9-N1357-N and | | | | | | | | | | |
| dSpCas9-N1357-C | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | — | 0.00% | 0.01% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | A | 0.02% | 0.02% | 0.01% | 0.00% | 0.01% | 99.83% | 0.00% | 0.01% | 0.01% |
| Cas9-N1357-C and | | | | | | | | | | |
| dSpCas9-N1357-N | | | | | | | | | | |
| nogRNA-N1357spilt-KKH- | T | 0.00% | 0.00% | 0.01% | 0.01% | 0.00% | 0.02% | 0.00% | 0.00% | 99.63% |
| Cas9-N1357-C and | | | | | | | | | | |
| dSpCas9-N1357-N | | | | | | | | | | |
| nogRNA-N1357spilt-KKH- | C | 0.01% | 0.01% | 99.97% | 99.98% | 99.98% | 0.07% | 0.00% | 0.00% | 0.03% |
| Cas9-N1357-C and | | | | | | | | | | |
| dSpCas9-N1357-N | | | | | | | | | | |
| nogRNA-N1357spilt-KKH- | G | 99.98% | 99.98% | 0.02% | 0.01% | 0.01% | 0.09% | 100.00% | 99.99% | 0.32% |
| Cas9-N1357-C and | | | | | | | | | | |
| dSpCas9-N1357-N | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1357spilt-KKH- | — | 0.00% | 0.00% | 0.00% | 0.00% | 0.01% | 0.00% | 0.00% | 0.00% | 0.01% |
| Cas9-N1357-C and | | | | | | | | | | |
| dSpCas9-N1357-N | | | | | | | | | | |
| nogRNA-N1387spilt-KKH- | A | 0.02% | 0.02% | 0.00% | 0.01% | 0.02% | 99.86% | 0.01% | 0.01% | 0.02% |
| Cas9-N1387-N and | | | | | | | | | | |
| dSpCas9-N1387-C | | | | | | | | | | |
| nogRNA-N1387spilt-KKH- | T | 0.01% | 0.02% | 0.03% | 0.02% | 0.03% | 0.02% | 0.01% | 0.00% | 99.69% |
| Cas9-N1387-N and | | | | | | | | | | |
| dSpCas9-N1387-C | | | | | | | | | | |
| nogRNA-N1387spilt-KKH- | C | 0.01% | 0.00% | 99.93% | 99.94% | 99.91% | 0.01% | 0.00% | 0.00% | 0.04% |
| Cas9-N1387-N and | | | | | | | | | | |
| dSpCas9-N1387-C | | | | | | | | | | |
| nogRNA-N1387spilt-KKH- | G | 99.92% | 99.93% | 0.00% | 0.00% | 0.00% | 0.08% | 99.96% | 99.98% | 0.25% |
| Cas9-N1387-N and | | | | | | | | | | |
| dSpCas9-N1387-C | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | — | 0.03% | 0.04% | 0.03% | 0.03% | 0.04% | 0.02% | 0.02% | 0.01% | 0.01% |
| Cas9-N1387-N and | | | | | | | | | | |
| dSpCas9-N1387-C | | | | | | | | | | |
| nogRNA-N1387spilt-KKH- | A | 0.02% | 0.01% | 0.00% | 0.01% | 0.01% | 99.91% | 0.00% | 0.00% | 0.01% |
| Cas9-N1387-C and | | | | | | | | | | |
| dSpCas9-N1387-N | | | | | | | | | | |
| nogRNA-N1387spilt-KKH- | T | 0.00% | 0.00% | 0.02% | 0.01% | 0.01% | 0.02% | 0.00% | 0.00% | 99.71% |
| Cas9-N1387-C and | | | | | | | | | | |
| dSpCas9-N1387-N | | | | | | | | | | |
| nogRNA-N1387spilt-KKH- | C | 0.00% | 0.00% | 99.96% | 99.97% | 99.96% | 0.03% | 0.00% | 0.00% | 0.06% |
| Cas9-N1387-C and | | | | | | | | | | |
| dSpCas9-N1387-N | | | | | | | | | | |
| nogRNA-N1387spilt-KKH- | G | 99.97% | 99.98% | 0.01% | 0.01% | 0.00% | 0.03% | 100.00% | 99.99% | 0.21% |
| Cas9-N1387-C and | | | | | | | | | | |
| dSpCas9-N1387-N | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |
| nogRNA-N1387spilt-KKH- | — | 0.00% | 0.00% | 0.00% | 0.00% | 0.01% | 0.00% | 0.00% | 0.00% | 0.00% |
| Cas9-N1387-C and | | | | | | | | | | |
| dSpCas9-N1387-N | | | | | | | | | | |

| Batch | Nucleotide | G | A | A | G | G | T | G | T |
|---|---|---|---|---|---|---|---|---|---|
| nogRNA-A1343spilt-KKH- | A | 0.01% | 99.95% | 99.93% | 0.00% | 0.00% | 0.02% | 0.01% | 0.02% |
| Cas9-A1343-N and | | | | | | | | | |
| dSpCas9-A1343-C | | | | | | | | | |
| nogRNA-A1343spilt-KKH- | T | 0.01% | 0.00% | 0.00% | 0.00% | 0.01% | 99.74% | 0.01% | 99.93% |
| Cas9-A1343-N and | | | | | | | | | |
| dSpCas9-A1343-C | | | | | | | | | |
| nogRNA-A1343spilt-KKH- | C | 0.00% | 0.02% | 0.00% | 0.00% | 0.00% | 0.05% | 0.00% | 0.01% |
| Cas9-A1343-N and | | | | | | | | | |
| dSpCas9-A1343-C | | | | | | | | | |
| nogRNA-A1343spilt-KKH- | G | 99.98% | 0.02% | 0.05% | 99.99% | 99.99% | 0.19% | 99.98% | 0.04% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | A | 0.00% | 99.86% | 99.83% | 0.01% | 0.01% | 0.02% | 0.01% | 0.00% |
| Cas9-A1343-C and | | | | | | | | | |
| dSpCas9-A1343-N | | | | | | | | | |
| nogRNA-A1343spilt-KKH- | T | 0.00% | 0.01% | 0.02% | 0.00% | 0.01% | 99.72% | 0.01% | 99.91% |
| Cas9-A1343-C and | | | | | | | | | |
| dSpCas9-A1343-N | | | | | | | | | |
| nogRNA-A1343spilt-KKH- | C | 0.00% | 0.07% | 0.07% | 0.00% | 0.00% | 0.05% | 0.00% | 0.01% |
| Cas9-A1343-C and | | | | | | | | | |
| dSpCas9-A1343-N | | | | | | | | | |
| nogRNA-A1343spilt-KKH- | G | 99.99% | 0.05% | 0.08% | 99.99% | 99.98% | 0.22% | 99.98% | 0.08% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-G1322spilt-KKH- | A | 0.01% | 99.93% | 99.87% | 0.01% | 0.00% | 0.03% | 0.00% | 0.01% |
| Cas9-G1322-N and | | | | | | | | | |
| dSpCas9-G1322-C | | | | | | | | | |
| nogRNA-G1322spilt-KKH- | T | 0.02% | 0.01% | 0.01% | 0.01% | 0.02% | 99.76% | 0.02% | 99.93% |
| Cas9-G1322-N and | | | | | | | | | |
| dSpCas9-G1322-C | | | | | | | | | |
| nogRNA-G1322spilt-KKH- | C | 0.00% | 0.03% | 0.03% | 0.00% | 0.00% | 0.04% | 0.00% | 0.03% |
| Cas9-G1322-N and | | | | | | | | | |
| dSpCas9-G1322-C | | | | | | | | | |
| nogRNA-G1322spilt-KKH- | G | 99.98% | 0.02% | 0.09% | 99.98% | 99.98% | 0.17% | 99.98% | 0.03% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | A | 0.00% | 99.90% | 99.85% | 0.00% | 0.00% | 0.02% | 0.00% | 0.01% |
| Cas9-G1322-C and | | | | | | | | | |
| dSpCas9-G1322-N | | | | | | | | | |
| nogRNA-G1322spilt-KKH- | T | 0.02% | 0.02% | 0.03% | 0.01% | 0.01% | 99.70% | 0.02% | 99.90% |
| Cas9-G1322-C and | | | | | | | | | |
| dSpCas9-G1322-N | | | | | | | | | |
| nogRNA-G1322spilt-KKH- | C | 0.01% | 0.03% | 0.01% | 0.01% | 0.00% | 0.07% | 0.00% | 0.03% |
| Cas9-G1322-C and | | | | | | | | | |
| dSpCas9-G1322-N | | | | | | | | | |
| nogRNA-G1322spilt-KKH- | G | 99.97% | 0.06% | 0.11% | 99.98% | 99.99% | 0.21% | 99.98% | 0.05% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1333spilt-KKH- | A | 0.00% | 99.95% | 99.89% | 0.00% | 0.01% | 0.03% | 0.00% | 0.01% |
| Cas9-G1333-N and | | | | | | | | | |
| dSpCas9-G1333-C | | | | | | | | | |
| nogRNA-G1333spilt-KKH- | T | 0.01% | 0.00% | 0.02% | 0.00% | 0.00% | 99.76% | 0.02% | 99.90% |
| Cas9-G1333-N and | | | | | | | | | |
| dSpCas9-G1333-C | | | | | | | | | |
| nogRNA-G1333spilt-KKH- | C | 0.00% | 0.02% | 0.03% | 0.00% | 0.00% | 0.05% | 0.00% | 0.03% |
| Cas9-G1333-N and | | | | | | | | | |
| dSpCas9-G1333-C | | | | | | | | | |
| nogRNA-G1333spilt-KKH- | G | 99.98% | 0.03% | 0.06% | 100.00% | 99.98% | 0.16% | 99.97% | 0.06% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | A | 0.00% | 99.91% | 99.86% | 0.00% | 0.01% | 0.01% | 0.01% | 0.01% |
| Cas9-G1333-C and | | | | | | | | | |
| dSpCas9-G1333-N | | | | | | | | | |
| nogRNA-G1333spilt-KKH- | T | 0.01% | 0.00% | 0.01% | 0.00% | 0.01% | 99.75% | 0.02% | 99.94% |
| Cas9-G1333-C and | | | | | | | | | |
| dSpCas9-G1333-N | | | | | | | | | |
| nogRNA-G1333spilt-KKH- | C | 0.00% | 0.05% | 0.04% | 0.00% | 0.00% | 0.06% | 0.00% | 0.01% |
| Cas9-G1333-C and | | | | | | | | | |
| dSpCas9-G1333-N | | | | | | | | | |
| nogRNA-G1333spilt-KKH- | G | 99.99% | 0.03% | 0.10% | 99.99% | 99.98% | 0.18% | 99.97% | 0.04% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1371spilt-KKH- | A | 0.01% | 99.92% | 99.90% | 0.02% | 0.02% | 0.03% | 0.02% | 0.02% |
| Cas9-G1371-N and | | | | | | | | | |
| dSpCas9-G1371-C | | | | | | | | | |
| nogRNA-G1371spilt-KKH- | T | 0.01% | 0.01% | 0.01% | 0.00% | 0.01% | 99.70% | 0.00% | 99.93% |
| Cas9-G1371-N and | | | | | | | | | |
| dSpCas9-G1371-C | | | | | | | | | |
| nogRNA-G1371spilt-KKH- | C | 0.00% | 0.03% | 0.03% | 0.00% | 0.00% | 0.05% | 0.00% | 0.02% |
| Cas9-G1371-N and | | | | | | | | | |
| dSpCas9-G1371-C | | | | | | | | | |
| nogRNA-G1371spilt-KKH- | G | 99.98% | 0.03% | 0.06% | 99.98% | 99.97% | 0.21% | 99.98% | 0.03% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | A | 0.00% | 99.88% | 99.85% | 0.00% | 0.01% | 0.04% | 0.00% | 0.01% |
| Cas9-G1371-C and | | | | | | | | | |
| dSpCas9-G1371-N | | | | | | | | | |
| nogRNA-G1371spilt-KKH- | T | 0.02% | 0.02% | 0.02% | 0.01% | 0.02% | 99.65% | 0.05% | 99.88% |
| Cas9-G1371-C and | | | | | | | | | |
| dSpCas9-G1371-N | | | | | | | | | |
| nogRNA-G1371spilt-KKH- | C | 0.00% | 0.05% | 0.02% | 0.00% | 0.00% | 0.08% | 0.00% | 0.04% |
| Cas9-G1371-C and | | | | | | | | | |
| dSpCas9-G1371-N | | | | | | | | | |
| nogRNA-G1371spilt-KKH- | G | 99.97% | 0.05% | 0.12% | 99.99% | 99.97% | 0.23% | 99.95% | 0.07% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1371spilt-KKH- | — | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1397spilt-KKH- | A | 0.00% | 99.72% | 99.73% | 0.00% | 0.00% | 0.03% | 0.01% | 0.02% |
| Cas9-G1397-N and | | | | | | | | | |
| dSpCas9-G1397-C | | | | | | | | | |
| nogRNA-G1397spilt-KKH- | T | 0.02% | 0.01% | 0.01% | 0.01% | 0.02% | 99.71% | 0.01% | 99.89% |
| Cas9-G1397-N and | | | | | | | | | |
| dSpCas9-G1397-C | | | | | | | | | |
| nogRNA-G1397spilt-KKH- | C | 0.00% | 0.03% | 0.02% | 0.00% | 0.00% | 0.05% | 0.00% | 0.02% |
| Cas9-G1397-N and | | | | | | | | | |
| dSpCas9-G1397-C | | | | | | | | | |
| nogRNA-G1397spilt-KKH- | G | 99.79% | 0.06% | 0.07% | 99.82% | 99.80% | 0.17% | 99.93% | 0.02% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.18% | 0.17% | 0.17% | 0.17% | 0.17% | 0.05% | 0.05% | 0.05% |
| Cas9-G1397-N and | | | | | | | | | |
| dSpCas9-G1397-C | | | | | | | | | |
| nogRNA-G1397spilt-KKH- | A | 0.00% | 99.92% | 99.90% | 0.01% | 0.00% | 0.03% | 0.01% | 0.00% |
| Cas9-G1397-C and | | | | | | | | | |
| dSpCas9-G1397-N | | | | | | | | | |
| nogRNA-G1397spilt-KKH- | T | 0.00% | 0.02% | 0.01% | 0.01% | 0.00% | 99.75% | 0.01% | 99.93% |
| Cas9-G1397-C and | | | | | | | | | |
| dSpCas9-G1397-N | | | | | | | | | |
| nogRNA-G1397spilt-KKH- | C | 0.00% | 0.02% | 0.02% | 0.00% | 0.00% | 0.06% | 0.00% | 0.03% |
| Cas9-G1397-C and | | | | | | | | | |
| dSpCas9-G1397-N | | | | | | | | | |
| nogRNA-G1397spilt-KKH- | G | 99.99% | 0.02% | 0.06% | 99.98% | 100.00% | 0.16% | 99.98% | 0.04% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.01% | 0.01% | 0.01% | 0.00% | 0.00% | 0.00% | 0.00% | 0.00% |
| Cas9-G1397-C and | | | | | | | | | |
| dSpCas9-G1397-N | | | | | | | | | |
| nogRNA-N1357spilt-KKH- | A | 0.00% | 99.90% | 99.89% | 0.00% | 0.00% | 0.05% | 0.01% | 0.00% |
| Cas9-N1357-N and | | | | | | | | | |
| dSpCas9-N1357-C | | | | | | | | | |
| nogRNA-N1357spilt-KKH- | T | 0.02% | 0.02% | 0.02% | 0.01% | 0.01% | 99.71% | 0.02% | 99.93% |
| Cas9-N1357-N and | | | | | | | | | |
| dSpCas9-N1357-C | | | | | | | | | |
| nogRNA-N1357spilt-KKH- | C | 0.00% | 0.05% | 0.02% | 0.00% | 0.00% | 0.06% | 0.00% | 0.02% |
| Cas9-N1357-N and | | | | | | | | | |
| dSpCas9-N1357-C | | | | | | | | | |
| nogRNA-N1357spilt-KKH- | G | 99.97% | 0.03% | 0.08% | 99.98% | 99.99% | 0.18% | 99.97% | 0.05% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.01% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | A | 0.01% | 99.87% | 99.79% | 0.00% | 0.00% | 0.02% | 0.00% | 0.00% |
| Cas9-N1357-C and | | | | | | | | | |
| dSpCas9-N1357-N | | | | | | | | | |
| nogRNA-N1357spilt-KKH- | T | 0.00% | 0.01% | 0.01% | 0.02% | 0.01% | 99.74% | 0.02% | 99.90% |
| Cas9-N1357-C and | | | | | | | | | |
| dSpCas9-N1357-N | | | | | | | | | |
| nogRNA-N1357spilt-KKH- | C | 0.00% | 0.07% | 0.06% | 0.00% | 0.01% | 0.03% | 0.00% | 0.03% |
| Cas9-N1357-C and | | | | | | | | | |
| dSpCas9-N1357-N | | | | | | | | | |
| nogRNA-N1357spilt-KKH- | G | 99.99% | 0.06% | 0.14% | 99.98% | 99.99% | 0.22% | 99.98% | 0.07% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1387spilt-KKH- | A | 0.02% | 99.89% | 99.85% | 0.01% | 0.01% | 0.02% | 0.01% | 0.01% |
| Cas9-N1387-N and | | | | | | | | | |
| dSpCas9-N1387-C | | | | | | | | | |
| nogRNA-N1387spilt-KKH- | T | 0.03% | 0.01% | 0.02% | 0.02% | 0.04% | 99.69% | 0.02% | 99.93% |
| Cas9-N1387-N and | | | | | | | | | |
| dSpCas9-N1387-C | | | | | | | | | |
| nogRNA-N1387spilt-KKH- | C | 0.00% | 0.06% | 0.03% | 0.00% | 0.00% | 0.06% | 0.00% | 0.01% |
| Cas9-N1387-N and | | | | | | | | | |
| dSpCas9-N1387-C | | | | | | | | | |
| nogRNA-N1387spilt-KKH- | G | 99.95% | 0.04% | 0.09% | 99.96% | 99.94% | 0.22% | 99.96% | 0.04% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.00% | 0.00% | 0.00% | 0.01% | 0.01% | 0.01% | 0.01% | 0.01% |
| Cas9-N1387-N and | | | | | | | | | |
| dSpCas9-N1387-C | | | | | | | | | |
| nogRNA-N1387spilt-KKH- | A | 0.00% | 99.91% | 99.89% | 0.00% | 0.00% | 0.02% | 0.00% | 0.00% |
| Cas9-N1387-C and | | | | | | | | | |
| dSpCas9-N1387-N | | | | | | | | | |
| nogRNA-N1387spilt-KKH- | T | 0.01% | 0.02% | 0.00% | 0.02% | 0.01% | 99.73% | 0.02% | 99.95% |
| Cas9-N1387-C and | | | | | | | | | |
| dSpCas9-N1387-N | | | | | | | | | |
| nogRNA-N1387spilt-KKH- | C | 0.00% | 0.04% | 0.02% | 0.00% | 0.00% | 0.07% | 0.00% | 0.02% |
| Cas9-N1387-C and | | | | | | | | | |
| dSpCas9-N1387-N | | | | | | | | | |
| nogRNA-N1387spilt-KKH- | G | 99.98% | 0.04% | 0.09% | 99.97% | 99.98% | 0.18% | 99.97% | 0.03% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |

| Batch | Nucleotide | G | G | T | T | C | C | A |
|---|---|---|---|---|---|---|---|---|
| nogRNA-A1343spilt-KKH- | A | 0.00% | 0.00% | 0.00% | 0.01% | 0.01% | 0.01% | 99.97% |
| Cas9-A1343-N and | | | | | | | | |
| dSpCas9-A1343-C | | | | | | | | |
| nogRNA-A1343spilt-KKH- | T | 0.01% | 0.00% | 99.96% | 99.98% | 0.00% | 0.01% | 0.00% |
| Cas9-A1343-N and | | | | | | | | |
| dSpCas9-A1343-C | | | | | | | | |
| nogRNA-A1343spilt-KKH- | C | 0.00% | 0.00% | 0.02% | 0.01% | 99.99% | 99.97% | 0.00% |
| Cas9-A1343-N and | | | | | | | | |
| dSpCas9-A1343-C | | | | | | | | |
| nogRNA-A1343spilt-KKH- | G | 99.99% | 99.99% | 0.02% | 0.00% | 0.00% | 0.00% | 0.02% |
| Cas9-A1343-N and | | | | | | | | |
| dSpCas9-A1343-C | | | | | | | | |
| nogRNA-A1343spilt-KKH- | N | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | — | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | A | 0.00% | 0.00% | 0.00% | 0.00% | 0.01% | 0.00% | 99.89% |
| Cas9-A1343-C and | | | | | | | | |
| dSpCas9-A1343-N | | | | | | | | |
| nogRNA-A1343spilt-KKH- | T | 0.02% | 0.00% | 99.95% | 99.99% | 0.02% | 0.01% | 0.01% |
| Cas9-A1343-C and | | | | | | | | |
| dSpCas9-A1343-N | | | | | | | | |
| nogRNA-A1343spilt-KKH- | C | 0.00% | 0.00% | 0.03% | 0.00% | 99.97% | 99.97% | 0.06% |
| Cas9-A1343-C and | | | | | | | | |
| dSpCas9-A1343-N | | | | | | | | |
| nogRNA-A1343spilt-KKH- | G | 99.97% | 100.00% | 0.02% | 0.00% | 0.00% | 0.00% | 0.04% |
| Cas9-A1343-C and | | | | | | | | |
| dSpCas9-A1343-N | | | | | | | | |
| nogRNA-A1343spilt-KKH- | N | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-A1343spilt-KKH- | — | 0.00% | 0.00% | 0.00% | 0.00% | 0.00% | 0.01% | 0.00% |
| Cas9-A1343-C and | | | | | | | | |
| dSpCas9-A1343-N | | | | | | | | |
| nogRNA-G1322spilt-KKH- | A | 0.02% | 0.01% | 0.00% | 0.00% | 0.00% | 0.01% | 99.94% |
| Cas9-G1322-N and | | | | | | | | |
| dSpCas9-G1322-C | | | | | | | | |
| nogRNA-G1322spilt-KKH- | T | 0.00% | 0.00% | 99.96% | 99.98% | 0.01% | 0.02% | 0.01% |
| Cas9-G1322-N and | | | | | | | | |
| dSpCas9-G1322-C | | | | | | | | |
| nogRNA-G1322spilt-KKH- | C | 0.00% | 0.01% | 0.02% | 0.01% | 99.99% | 99.95% | 0.01% |
| Cas9-G1322-N and | | | | | | | | |
| dSpCas9-G1322-C | | | | | | | | |
| nogRNA-G1322spilt-KKH- | G | 99.98% | 99.98% | 0.02% | 0.00% | 0.00% | 0.00% | 0.03% |
| Cas9-G1322-N and | | | | | | | | |
| dSpCas9-G1322-C | | | | | | | | |
| nogRNA-G1322spilt-KKH- | N | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | — | 0.00% | 0.00% | 0.00% | 0.00% | 0.00% | 0.01% | 0.00% |
| Cas9-G1322-N and | | | | | | | | |
| dSpCas9-G1322-C | | | | | | | | |
| nogRNA-G1322spilt-KKH- | A | 0.01% | 0.00% | 0.00% | 0.02% | 0.01% | 0.00% | 99.97% |
| Cas9-G1322-C and | | | | | | | | |
| dSpCas9-G1322-N | | | | | | | | |
| nogRNA-G1322spilt-KKH- | T | 0.01% | 0.00% | 99.95% | 99.97% | 0.05% | 0.02% | 0.01% |
| Cas9-G1322-C and | | | | | | | | |
| dSpCas9-G1322-N | | | | | | | | |
| nogRNA-G1322spilt-KKH- | C | 0.00% | 0.00% | 0.01% | 0.01% | 99.93% | 99.97% | 0.01% |
| Cas9-G1322-C and | | | | | | | | |
| dSpCas9-G1322-N | | | | | | | | |
| nogRNA-G1322spilt-KKH- | G | 99.97% | 99.99% | 0.03% | 0.00% | 0.00% | 0.00% | 0.02% |
| Cas9-G1322-C and | | | | | | | | |
| dSpCas9-G1322-N | | | | | | | | |
| nogRNA-G1322spilt-KKH- | N | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1322spilt-KKH- | — | 0.00% | 0.00% | 0.00% | 0.00% | 0.00% | 0.01% | 0.00% |
| Cas9-G1322-C and | | | | | | | | |
| dSpCas9-G1322-N | | | | | | | | |
| nogRNA-G1333spilt-KKH- | A | 0.00% | 0.00% | 0.00% | 0.01% | 0.00% | 0.01% | 99.95% |
| Cas9-G1333-N and | | | | | | | | |
| dSpCas9-G1333-C | | | | | | | | |
| nogRNA-G1333spilt-KKH- | T | 0.00% | 0.00% | 99.96% | 99.94% | 0.03% | 0.00% | 0.00% |
| Cas9-G1333-N and | | | | | | | | |
| dSpCas9-G1333-C | | | | | | | | |
| nogRNA-G1333spilt-KKH- | C | 0.00% | 0.00% | 0.02% | 0.04% | 99.97% | 99.98% | 0.01% |
| Cas9-G1333-N and | | | | | | | | |
| dSpCas9-G1333-C | | | | | | | | |
| nogRNA-G1333spilt-KKH- | G | 10.000% | 100.00% | 0.02% | 0.00% | 0.00% | 0.00% | 0.04% |
| Cas9-G1333-N and | | | | | | | | |
| dSpCas9-G1333-C | | | | | | | | |
| nogRNA-G1333spilt-KKH- | N | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | — | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | A | 0.00% | 0.01% | 0.00% | 0.00% | 0.00% | 0.00% | 99.96% |
| Cas9-G1333-C and | | | | | | | | |
| dSpCas9-G1333-N | | | | | | | | |
| nogRNA-G1333spilt-KKH- | T | 0.01% | 0.00% | 99.96% | 99.98% | 0.02% | 0.03% | 0.00% |
| Cas9-G1333-C and | | | | | | | | |
| dSpCas9-G1333-N | | | | | | | | |
| nogRNA-G1333spilt-KKH- | C | 0.00% | 0.00% | 0.02% | 0.01% | 99.98% | 99.95% | 0.02% |
| Cas9-G1333-C and | | | | | | | | |
| dSpCas9-G1333-N | | | | | | | | |
| nogRNA-G1333spilt-KKH- | G | 99.99% | 99.99% | 0.02% | 0.00% | 0.00% | 0.00% | 0.01% |
| Cas9-G1333-C and | | | | | | | | |
| dSpCas9-G1333-N | | | | | | | | |
| nogRNA-G1333spilt-KKH- | N | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1333spilt-KKH- | — | 0.00% | 0.00% | 0.00% | 0.00% | 0.00% | 0.01% | 0.00% |
| Cas9-G1333-C and | | | | | | | | |
| dSpCas9-G1333-N | | | | | | | | |
| nogRNA-G1371spilt-KKH- | A | 0.00% | 0.01% | 0.02% | 0.01% | 0.00% | 0.01% | 99.93% |
| Cas9-G1371-N and | | | | | | | | |
| dSpCas9-G1371-C | | | | | | | | |
| nogRNA-G1371spilt-KKH- | T | 0.02% | 0.00% | 99.95% | 99.98% | 0.02% | 0.01% | 0.02% |
| Cas9-G1371-N and | | | | | | | | |
| dSpCas9-G1371-C | | | | | | | | |
| nogRNA-G1371spilt-KKH- | C | 0.00% | 0.00% | 0.02% | 0.01% | 99.98% | 99.97% | 0.01% |
| Cas9-G1371-N and | | | | | | | | |
| dSpCas9-G1371-C | | | | | | | | |
| nogRNA-G1371spilt-KKH- | G | 99.98% | 99.98% | 0.02% | 0.00% | 0.00% | 0.00% | 0.03% |
| Cas9-G1371-N and | | | | | | | | |
| dSpCas9-G1371-C | | | | | | | | |
| nogRNA-G1371spilt-KKH- | N | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | — | 0.00% | 0.00% | 0.00% | 0.00% | 0.00% | 0.01% | 0.00% |
| Cas9-G1371-N and | | | | | | | | |
| dSpCas9-G1371-C | | | | | | | | |
| nogRNA-G1371spilt-KKH- | A | 0.00% | 0.02% | 0.00% | 0.01% | 0.00% | 0.01% | 99.92% |
| Cas9-G1371-C and | | | | | | | | |
| dSpCas9-G1371-N | | | | | | | | |
| nogRNA-G1371spilt-KKH- | T | 0.00% | 0.00% | 99.96% | 99.96% | 0.02% | 0.03% | 0.01% |
| Cas9-G1371-C and | | | | | | | | |
| dSpCas9-G1371-N | | | | | | | | |
| nogRNA-G1371spilt-KKH- | C | 0.00% | 0.00% | 0.01% | 0.02% | 99.97% | 99.95% | 0.02% |
| Cas9-G1371-C and | | | | | | | | |
| dSpCas9-G1371-N | | | | | | | | |
| nogRNA-G1371spilt-KKH- | G | 99.99% | 99.98% | 0.02% | 0.00% | 0.00% | 0.00% | 0.06% |
| Cas9-G1371-C and | | | | | | | | |
| dSpCas9-G1371-N | | | | | | | | |
| nogRNA-G1371spilt-KKH- | N | 0.00% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1371spilt-KKH- | — | 0.00% | 0.00% | 0.00% | 0.01% | 0.00% | 0.01% | 0.00% |
| Cas9-G1371-C and | | | | | | | | |
| dSpCas9-G1371-N | | | | | | | | |
| nogRNA-G1397spilt-KKH- | A | 0.00% | 0.01% | 0.01% | 0.00% | 0.01% | 0.02% | 99.94% |
| Cas9-G1397-N and | | | | | | | | |
| dSpCas9-G1397-C | | | | | | | | |
| nogRNA-G1397spilt-KKH- | T | 0.00% | 0.00% | 99.95% | 99.96% | 0.01% | 0.02% | 0.01% |
| Cas9-G1397-N and | | | | | | | | |
| dSpCas9-G1397-C | | | | | | | | |
| nogRNA-G1397spilt-KKH- | C | 0.00% | 0.00% | 0.02% | 0.03% | 99.98% | 99.95% | 0.00% |
| Cas9-G1397-N and | | | | | | | | |
| dSpCas9-G1397-C | | | | | | | | |
| nogRNA-G1397spilt-KKH- | G | 99.95% | 99.94% | 0.01% | 0.00% | 0.00% | 0.00% | 0.04% |
| Cas9-G1397-N and | | | | | | | | |
| dSpCas9-G1397-C | | | | | | | | |
| nogRNA-G1397spilt-KKH- | N | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | — | 0.05% | 0.05% | 0.00% | 0.00% | 0.00% | 0.01% | 0.00% |
| Cas9-G1397-N and | | | | | | | | |
| dSpCas9-G1397-C | | | | | | | | |
| nogRNA-G1397spilt-KKH- | A | 0.00% | 0.01% | 0.00% | 0.01% | 0.01% | 0.01% | 99.95% |
| Cas9-G1397-C and | | | | | | | | |
| dSpCas9-G1397-N | | | | | | | | |
| nogRNA-G1397spilt-KKH- | T | 0.01% | 0.00% | 99.95% | 99.96% | 0.02% | 0.01% | 0.01% |
| Cas9-G1397-C and | | | | | | | | |
| dSpCas9-G1397-N | | | | | | | | |
| nogRNA-G1397spilt-KKH- | C | 0.00% | 0.00% | 0.03% | 0.02% | 99.97% | 99.94% | 0.00% |
| Cas9-G1397-C and | | | | | | | | |
| dSpCas9-G1397-N | | | | | | | | |
| nogRNA-G1397spilt-KKH- | G | 99.99% | 99.99% | 0.02% | 0.01% | 0.00% | 0.01% | 0.03% |
| Cas9-G1397-C and | | | | | | | | |
| dSpCas9-G1397-N | | | | | | | | |
| nogRNA-G1397spilt-KKH- | N | 0.00% | 0.00% | 0.00% | 0.00% | 0.00% | 0.00% | 0.00% |
| Cas9-G1397-C and | | | | | | | | |
| dSpCas9-G1397-N | | | | | | | | |
| nogRNA-G1397spilt-KKH- | — | 0.00% | 0.00% | 0.00% | 0.00% | 0.00% | 0.02% | 0.00% |
| Cas9-G1397-C and | | | | | | | | |
| dSpCas9-G1397-N | | | | | | | | |
| nogRNA-N1357spilt-KKH- | A | 0.01% | 0.01% | 0.01% | 0.01% | 0.00% | 0.00% | 99.97% |
| Cas9-N1357-N and | | | | | | | | |
| dSpCas9-N1357-C | | | | | | | | |
| nogRNA-N1357spilt-KKH- | T | 0.01% | 0.00% | 99.97% | 99.97% | 0.01% | 0.01% | 0.01% |
| Cas9-N1357-N and | | | | | | | | |
| dSpCas9-N1357-C | | | | | | | | |
| nogRNA-N1357spilt-KKH- | C | 0.00% | 0.00% | 0.00% | 0.02% | 99.98% | 99.98% | 0.00% |
| Cas9-N1357-N and | | | | | | | | |
| dSpCas9-N1357-C | | | | | | | | |
| nogRNA-N1357spilt-KKH- | G | 99.98% | 99.98% | 0.02% | 0.00% | 0.00% | 0.00% | 0.02% |
| Cas9-N1357-N and | | | | | | | | |
| dSpCas9-N1357-C | | | | | | | | |
| nogRNA-N1357spilt-KKH- | N | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | — | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | A | 0.00% | 0.02% | 0.00% | 0.01% | 0.00% | 0.01% | 99.91% |
| Cas9-N1357-C and | | | | | | | | |
| dSpCas9-N1357-N | | | | | | | | |
| nogRNA-N1357spilt-KKH- | T | 0.02% | 0.00% | 99.97% | 99.97% | 0.02% | 0.02% | 0.01% |
| Cas9-N1357-C and | | | | | | | | |
| dSpCas9-N1357-N | | | | | | | | |
| nogRNA-N1357spilt-KKH- | C | 0.00% | 0.00% | 0.02% | 0.02% | 99.98% | 99.97% | 0.06% |
| Cas9-N1357-C and | | | | | | | | |
| dSpCas9-N1357-N | | | | | | | | |
| nogRNA-N1357spilt-KKH- | G | 99.98% | 99.98% | 0.02% | 0.00% | 0.00% | 0.00% | 0.03% |
| Cas9-N1357-C and | | | | | | | | |
| dSpCas9-N1357-N | | | | | | | | |
| nogRNA-N1357spilt-KKH- | N | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1357spilt-KKH- | — | 0.00% | 0.01% | 0.00% | 0.01% | 0.00% | 0.01% | 0.00% |
| Cas9-N1357-C and | | | | | | | | |
| dSpCas9-N1357-N | | | | | | | | |
| nogRNA-N1387spilt-KKH- | A | 0.01% | 0.00% | 0.01% | 0.01% | 0.00% | 0.04% | 99.95% |
| Cas9-N1387-N and | | | | | | | | |
| dSpCas9-N1387-C | | | | | | | | |
| nogRNA-N1387spilt-KKH- | T | 0.00% | 0.01% | 99.96% | 99.95% | 0.01% | 0.01% | 0.00% |
| Cas9-N1387-N and | | | | | | | | |
| dSpCas9-N1387-C | | | | | | | | |
| nogRNA-N1387spilt-KKH- | C | 0.00% | 0.01% | 0.01% | 0.01% | 99.98% | 99.94% | 0.00% |
| Cas9-N1387-N and | | | | | | | | |
| dSpCas9-N1387-C | | | | | | | | |
| nogRNA-N1387spilt-KKH- | G | 99.97% | 99.96% | 0.01% | 0.01% | 0.00% | 0.00% | 0.04% |
| Cas9-N1387-N and | | | | | | | | |
| dSpCas9-N1387-C | | | | | | | | |
| nogRNA-N1387spilt-KKH- | N | 0.00% | 0.00% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | — | 0.01% | 0.01% | 0.01% | 0.01% | 0.01% | 0.01% | 0.00% |
| Cas9-N1387-N and | | | | | | | | |
| dSpCas9-N1387-C | | | | | | | | |
| nogRNA-N1387spilt-KKH- | A | 0.01% | 0.00% | 0.00% | 0.00% | 0.00% | 0.00% | 99.97% |
| Cas9-N1387-C and | | | | | | | | |
| dSpCas9-N1387-N | | | | | | | | |
| nogRNA-N1387spilt-KKH- | T | 0.01% | 0.01% | 99.97% | 99.96% | 0.02% | 0.01% | 0.00% |
| Cas9-N1387-C and | | | | | | | | |
| dSpCas9-N1387-N | | | | | | | | |
| nogRNA-N1387spilt-KKH- | C | 0.00% | 0.00% | 0.01% | 0.03% | 99.97% | 99.97% | 0.00% |
| Cas9-N1387-C and | | | | | | | | |
| dSpCas9-N1387-N | | | | | | | | |
| nogRNA-N1387spilt-KKH- | G | 99.98% | 99.99% | 0.02% | 0.00% | 0.00% | 0.00% | 0.03% |
| Cas9-N1387-C and | | | | | | | | |
| dSpCas9-N1387-N | | | | | | | | |
| nogRNA-N1387spilt-KKH- | N | 0.00% | 0.00% | 0.00% | 0.00% | 0.00% | 0.00% | 0.00% |
| Cas9-N1387-C and | | | | | | | | |
| dSpCas9-N1387-N | | | | | | | | |
| nogRNA-N1387spilt-KKH- | — | 0.00% | 0.00% | 0.00% | 0.00% | 0.00% | 0.01% | 0.00% |
| Cas9-N1387-C and | | | | | | | | |
| dSpCas9-N1387-N | | | | | | | | |

| Batch | Nucleotide | G | A | A | C | C | G | G | A | G |
|---|---|---|---|---|---|---|---|---|---|---|
| nogRNA-A1343spilt-KKH- | A | 0.00% | 99.98% | 99.96% | 0.01% | 0.00% | 0.00% | 0.01% | 99.92% | 0.00% |
| Cas9-A1343-N and | | | | | | | | | | |
| dSpCas9-A1343-C | | | | | | | | | | |
| nogRNA-A1343spilt-KKH- | T | 0.01% | 0.00% | 0.00% | 0.01% | 0.03% | 0.01% | 0.00% | 0.01% | 0.02% |
| Cas9-A1343-N and | | | | | | | | | | |
| dSpCas9-A1343-C | | | | | | | | | | |
| nogRNA-A1343spilt-KKH- | C | 0.00% | 0.01% | 0.02% | 99.98% | 99.96% | 0.00% | 0.00% | 0.01% | 0.00% |
| Cas9-A1343-N and | | | | | | | | | | |
| dSpCas9-A1343-C | | | | | | | | | | |
| nogRNA-A1343spilt-KKH- | G | 99.98% | 0.00% | 0.02% | 0.00% | 0.00% | 99.98% | 99.98% | 0.05% | 99.98% |
| Cas9-A1343-N and | | | | | | | | | | |
| dSpCas9-A1343-C | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | A | 0.02% | 99.91% | 99.92% | 0.00% | 0.00% | 0.01% | 0.00% | 99.83% | 0.00% |
| Cas9-A1343-C and | | | | | | | | | | |
| dSpCas9-A1343-N | | | | | | | | | | |
| nogRNA-A1343spilt-KKH- | T | 0.00% | 0.00% | 0.00% | 0.00% | 0.01% | 0.02% | 0.01% | 0.00% | 0.00% |
| Cas9-A1343-C and | | | | | | | | | | |
| dSpCas9-A1343-N | | | | | | | | | | |
| nogRNA-A1343spilt-KKH- | C | 0.00% | 0.07% | 0.06% | 99.99% | 99.98% | 0.01% | 0.00% | 0.08% | 0.00% |
| Cas9-A1343-C and | | | | | | | | | | |
| dSpCas9-A1343-N | | | | | | | | | | |
| nogRNA-A1343spilt-KKH- | G | 99.98% | 0.02% | 0.01% | 0.00% | 0.01% | 99.96% | 99.98% | 0.09% | 99.99% |
| Cas9-A1343-C and | | | | | | | | | | |
| dSpCas9-A1343-N | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-G1322spilt-KKH- | A | 0.00% | 99.96% | 99.98% | 0.01% | 0.00% | 0.01% | 0.01% | 99.90% | 0.00% |
| Cas9-G1322-N and | | | | | | | | | | |
| dSpCas9-G1322-C | | | | | | | | | | |
| nogRNA-G1322spilt-KKH- | T | 0.00% | 0.00% | 0.00% | 0.01% | 0.02% | 0.00% | 0.01% | 0.02% | 0.02% |
| Cas9-G1322-N and | | | | | | | | | | |
| dSpCas9-G1322-C | | | | | | | | | | |
| nogRNA-G1322spilt-KKH- | C | 0.00% | 0.02% | 0.01% | 99.99% | 99.98% | 0.00% | 0.00% | 0.02% | 0.00% |
| Cas9-G1322-N and | | | | | | | | | | |
| dSpCas9-G1322-C | | | | | | | | | | |
| nogRNA-G1322spilt-KKH- | G | 99.99% | 0.01% | 0.01% | 0.00% | 0.01% | 99.98% | 99.98% | 0.06% | 99.98% |
| Cas9-G1322-N and | | | | | | | | | | |
| dSpCas9-G1322-C | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | A | 0.01% | 99.97% | 99.96% | 0.00% | 0.01% | 0.03% | 0.01% | 99.92% | 0.00% |
| Cas9-G1322-C and | | | | | | | | | | |
| dSpCas9-G1322-N | | | | | | | | | | |
| nogRNA-G1322spilt-KKH- | T | 0.00% | 0.00% | 0.01% | 0.00% | 0.03% | 0.01% | 0.01% | 0.02% | 0.01% |
| Cas9-G1322-C and | | | | | | | | | | |
| dSpCas9-G1322-N | | | | | | | | | | |
| nogRNA-G1322spilt-KKH- | C | 0.00% | 0.00% | 0.00% | 99.99% | 99.95% | 0.00% | 0.00% | 0.02% | 0.00% |
| Cas9-G1322-C and | | | | | | | | | | |
| dSpCas9-G1322-N | | | | | | | | | | |
| nogRNA-G1322spilt-KKH- | G | 99.98% | 0.02% | 0.02% | 0.00% | 0.01% | 99.96% | 99.97% | 0.05% | 99.98% |
| Cas9-G1322-C and | | | | | | | | | | |
| dSpCas9-G1322-N | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1333spilt-KKH- | A | 0.00% | 99.96% | 99.98% | 0.00% | 0.00% | 0.02% | 0.02% | 99.88% | 0.01% |
| Cas9-G1333-N and | | | | | | | | | | |
| dSpCas9-G1333-C | | | | | | | | | | |
| nogRNA-G1333spilt-KKH- | T | 0.00% | 0.00% | 0.00% | 0.01% | 0.00% | 0.01% | 0.00% | 0.02% | 0.01% |
| Cas9-G1333-N and | | | | | | | | | | |
| dSpCas9-G1333-C | | | | | | | | | | |
| nogRNA-G1333spilt-KKH- | C | 0.00% | 0.03% | 0.00% | 99.98% | 99.99% | 0.00% | 0.00% | 0.04% | 0.00% |
| Cas9-G1333-N and | | | | | | | | | | |
| dSpCas9-G1333-C | | | | | | | | | | |
| nogRNA-G1333spilt-KKH- | G | 100.00% | 0.01% | 0.00% | 0.00% | 0.01% | 99.97% | 99.98% | 0.06% | 99.98% |
| Cas9-G1333-N and | | | | | | | | | | |
| dSpCas9-G1333-C | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | A | 0.00% | 99.96% | 99.97% | 0.00% | 0.00% | 0.02% | 0.01% | 99.90% | 0.02% |
| Cas9-G1333-C and | | | | | | | | | | |
| dSpCas9-G1333-N | | | | | | | | | | |
| nogRNA-G1333spilt-KKH- | T | 0.01% | 0.00% | 0.00% | 0.00% | 0.00% | 0.03% | 0.01% | 0.02% | 0.02% |
| Cas9-G1333-C and | | | | | | | | | | |
| dSpCas9-G1333-N | | | | | | | | | | |
| nogRNA-G1333spilt-KKH- | C | 0.00% | 0.02% | 0.01% | 99.99% | 100.00% | 0.00% | 0.00% | 0.03% | 0.00% |
| Cas9-G1333-C and | | | | | | | | | | |
| dSpCas9-G1333-N | | | | | | | | | | |
| nogRNA-G1333spilt-KKH- | G | 99.99% | 0.02% | 0.02% | 0.00% | 0.00% | 99.95% | 99.97% | 0.05% | 99.96% |
| Cas9-G1333-C and | | | | | | | | | | |
| dSpCas9-G1333-N | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1371spilt-KKH- | A | 0.01% | 99.94% | 99.97% | 0.00% | 0.00% | 0.01% | 0.01% | 99.87% | 0.00% |
| Cas9-G1371-N and | | | | | | | | | | |
| dSpCas9-G1371-C | | | | | | | | | | |
| nogRNA-G1371spilt-KKH- | T | 0.01% | 0.00% | 0.01% | 0.00% | 0.02% | 0.01% | 0.00% | 0.00% | 0.01% |
| Cas9-G1371-N and | | | | | | | | | | |
| dSpCas9-G1371-C | | | | | | | | | | |
| nogRNA-G1371spilt-KKH- | C | 0.00% | 0.02% | 0.01% | 99.99% | 99.98% | 0.00% | 0.00% | 0.05% | 0.00% |
| Cas9-G1371-N and | | | | | | | | | | |
| dSpCas9-G1371-C | | | | | | | | | | |
| nogRNA-G1371spilt-KKH- | G | 99.98% | 0.04% | 0.02% | 0.00% | 0.00% | 99.98% | 99.98% | 0.07% | 99.98% |
| Cas9-G1371-N and | | | | | | | | | | |
| dSpCas9-G1371-C | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | — | 0.00% | 0.00% | 0.00% | 0.00% | 0.00% | 0.00% | 0.01% | 0.01% | 0.00% |
| Cas9-G1371-N and | | | | | | | | | | |
| dSpCas9-G1371-C | | | | | | | | | | |
| nogRNA-G1371spilt-KKH- | A | 0.01% | 99.96% | 99.96% | 0.00% | 0.00% | 0.00% | 0.01% | 99.88% | 0.01% |
| Cas9-G1371-C and | | | | | | | | | | |
| dSpCas9-G1371-N | | | | | | | | | | |
| nogRNA-G1371spilt-KKH- | T | 0.01% | 0.00% | 0.01% | 0.01% | 0.02% | 0.01% | 0.00% | 0.01% | 0.01% |
| Cas9-G1371-C and | | | | | | | | | | |
| dSpCas9-G1371-N | | | | | | | | | | |
| nogRNA-G1371spilt-KKH- | C | 0.00% | 0.01% | 0.01% | 99.98% | 99.98% | 0.00% | 0.00% | 0.04% | 0.00% |
| Cas9-G1371-C and | | | | | | | | | | |
| dSpCas9-G1371-N | | | | | | | | | | |
| nogRNA-G1371spilt-KKH- | G | 99.98% | 0.03% | 0.01% | 0.00% | 0.00% | 99.99% | 99.98% | 0.07% | 99.98% |
| Cas9-G1371-C and | | | | | | | | | | |
| dSpCas9-G1371-N | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-C and | | | | | | | | | | |
| dSnCas9-G1371-N | | | | | | | | | | |
| nogRNA-G1371spilt-KKH- | — | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1397spilt-KKH- | A | 0.00% | 99.98% | 99.98% | 0.00% | 0.00% | 0.02% | 0.01% | 99.92% | 0.01% |
| Cas9-G1397-N and | | | | | | | | | | |
| dSpCas9-G1397-C | | | | | | | | | | |
| nogRNA-G1397spilt-KKH- | T | 0.00% | 0.00% | 0.00% | 0.00% | 0.03% | 0.02% | 0.01% | 0.02% | 0.00% |
| Cas9-G1397-N and | | | | | | | | | | |
| dSpCas9-G1397-C | | | | | | | | | | |
| nogRNA-G1397spilt-KKH- | C | 0.00% | 0.01% | 0.01% | 100.00% | 99.95% | 0.01% | 0.00% | 0.02% | 0.00% |
| Cas9-G1397-N and | | | | | | | | | | |
| dSpCas9-G1397-C | | | | | | | | | | |
| nogRNA-G1397spilt-KKH- | G | 99.99% | 0.01% | 0.00% | 0.00% | 0.01% | 99.95% | 99.98% | 0.03% | 99.99% |
| Cas9-G1397-N and | | | | | | | | | | |
| dSpCas9-G1397-C | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | — | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | A | 0.01% | 99.98% | 99.96% | 0.00% | 0.01% | 0.01% | 0.01% | 99.91% | 0.00% |
| Cas9-G1397-C and | | | | | | | | | | |
| dSpCas9-G1397-N | | | | | | | | | | |
| nogRNA-G1397spilt-KKH- | T | 0.01% | 0.00% | 0.01% | 0.00% | 0.02% | 0.01% | 0.01% | 0.02% | 0.01% |
| Cas9-G1397-C and | | | | | | | | | | |
| dSpCas9-G1397-N | | | | | | | | | | |
| nogRNA-G1397spilt-KKH- | C | 0.00% | 0.01% | 0.01% | 99.99% | 99.95% | 0.00% | 0.00% | 0.01% | 0.00% |
| Cas9-G1397-C and | | | | | | | | | | |
| dSpCas9-G1397-N | | | | | | | | | | |
| nogRNA-G1397spilt-KKH- | G | 99.97% | 0.01% | 0.02% | 0.00% | 0.01% | 99.98% | 99.98% | 0.06% | 99.99% |
| Cas9-G1397-C and | | | | | | | | | | |
| dSpCas9-G1397-N | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-G1397spilt-KKH- | — | 0.01% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-N1357spilt-KKH- | A | 0.01% | 99.97% | 99.96% | 0.00% | 0.00% | 0.02% | 0.01% | 99.93% | 0.01% |
| Cas9-N1357-N and | | | | | | | | | | |
| dSpCas9-N1357-C | | | | | | | | | | |
| nogRNA-N1357spilt-KKH- | T | 0.00% | 0.01% | 0.01% | 0.00% | 0.01% | 0.01% | 0.01% | 0.02% | 0.00% |
| Cas9-N1357-N and | | | | | | | | | | |
| dSpCas9-N1357-C | | | | | | | | | | |
| nogRNA-N1357spilt-KKH- | C | 0.00% | 0.01% | 0.02% | 100.00% | 99.98% | 0.00% | 0.00% | 0.03% | 0.00% |
| Cas9-N1357-N and | | | | | | | | | | |
| dSpCas9-N1357-C | | | | | | | | | | |
| nogRNA-N1357spilt-KKH- | G | 99.98% | 0.01% | 0.02% | 0.00% | 0.00% | 99.97% | 99.98% | 0.03% | 99.98% |
| Cas9-N1357-N and | | | | | | | | | | |
| dSpCas9-N1357-C | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | A | 0.01% | 99.90% | 99.94% | 0.00% | 0.00% | 0.01% | 0.01% | 99.85% | 0.01% |
| Cas9-N1357-C and | | | | | | | | | | |
| dSpCas9-N1357-N | | | | | | | | | | |
| nogRNA-N1357spilt-KKH- | T | 0.01% | 0.00% | 0.00% | 0.01% | 0.02% | 0.03% | 0.01% | 0.01% | 0.01% |
| Cas9-N1357-C and | | | | | | | | | | |
| dSpCas9-N1357-N | | | | | | | | | | |
| nogRNA-N1357spilt-KKH- | C | 0.00% | 0.05% | 0.06% | 99.98% | 99.98% | 0.01% | 0.01% | 0.07% | 0.00% |
| Cas9-N1357-C and | | | | | | | | | | |
| dSpCas9-N1357-N | | | | | | | | | | |
| nogRNA-N1357spilt-KKH- | G | 99.98% | 0.05% | 0.01% | 0.01% | 0.00% | 99.95% | 99.97% | 0.08% | 99.99% |
| Cas9-N1357-C and | | | | | | | | | | |
| dSpCas9-N1357-N | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1357spilt-KKH- | — | 0.00% | 0.00% | 0.00% | 0.00% | 0.00% | 0.01% | 0.01% | 0.00% | 0.00% |
| Cas9-N1357-C and | | | | | | | | | | |
| dSpCas9-N1357-N | | | | | | | | | | |
| nogRNA-N1387spilt-KKH- | A | 0.01% | 99.97% | 99.96% | 0.01% | 0.00% | 0.01% | 0.02% | 99.85% | 0.00% |
| Cas9-N1387-N and | | | | | | | | | | |
| dSpCas9-N1387-C | | | | | | | | | | |
| nogRNA-N1387spilt-KKH- | T | 0.01% | 0.00% | 0.00% | 0.01% | 0.01% | 0.01% | 0.00% | 0.03% | 0.01% |
| Cas9-N1387-N and | | | | | | | | | | |
| dSpCas9-N1387-C | | | | | | | | | | |
| nogRNA-N1387spilt-KKH- | C | 0.00% | 0.01% | 0.01% | 99.98% | 99.96% | 0.00% | 0.00% | 0.03% | 0.00% |
| Cas9-N1387-N and | | | | | | | | | | |
| dSpCas9-N1387-C | | | | | | | | | | |
| nogRNA-N1387spilt-KKH- | G | 99.98% | 0.01% | 0.03% | 0.00% | 0.02% | 99.97% | 99.97% | 0.09% | 99.98% |
| Cas9-N1387-N and | | | | | | | | | | |
| dSpCas9-N1387-C | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | — | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | A | 0.00% | 99.99% | 99.98% | 0.00% | 0.00% | 0.00% | 0.02% | 99.92% | 0.01% |
| Cas9-N1387-C and | | | | | | | | | | |
| dSpCas9-N1387-N | | | | | | | | | | |
| nogRNA-N1387spilt-KKH- | T | 0.00% | 0.00% | 0.00% | 0.00% | 0.01% | 0.01% | 0.01% | 0.01% | 0.01% |
| Cas9-N1387-C and | | | | | | | | | | |
| dSpCas9-N1387-N | | | | | | | | | | |
| nogRNA-N1387spilt-KKH- | C | 0.00% | 0.01% | 0.01% | 100.00% | 99.98% | 0.00% | 0.00% | 0.02% | 0.00% |
| Cas9-N1387-C and | | | | | | | | | | |
| dSpCas9-N1387-N | | | | | | | | | | |
| nogRNA-N1387spilt-KKH- | G | 99.99% | 0.00% | 0.01% | 0.00% | 0.00% | 99.99% | 99.97% | 0.04% | 99.98% |
| Cas9-N1387-C and | | | | | | | | | | |
| dSpCas9-N1387-N | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |
| nogRNA-N1387spilt-KKH- | — | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |

| Batch | Nucleotide | G | A | C | A | A | A | G | T |
|---|---|---|---|---|---|---|---|---|---|
| nogRNA-A1343spilt-KKH- | A | 0.00% | 99.92% | 0.00% | 99.92% | 99.92% | 99.89% | 0.00% | 0.07% |
| Cas9-A1343-N and | | | | | | | | | |
| dSpCas9-A1343-C | | | | | | | | | |
| nogRNA-A1343spilt-KKH- | T | 0.01% | 0.02% | 0.00% | 0.01% | 0.01% | 0.00% | 0.02% | 99.86% |
| Cas9-A1343-N and | | | | | | | | | |
| dSpCas9-A1343-C | | | | | | | | | |
| nogRNA-A1343spilt-KKH- | C | 0.01% | 0.04% | 99.99% | 0.04% | 0.05% | 0.06% | 0.00% | 0.03% |
| Cas9-A1343-N and | | | | | | | | | |
| dSpCas9-A1343-C | | | | | | | | | |
| nogRNA-A1343spilt-KKH- | G | 99.98% | 0.02% | 0.00% | 0.03% | 0.02% | 0.04% | 99.98% | 0.04% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | A | 0.00% | 99.83% | 0.00% | 99.87% | 99.86% | 99.88% | 0.00% | 0.08% |
| Cas9-A1343-C and | | | | | | | | | |
| dSpCas9-A1343-N | | | | | | | | | |
| nogRNA-A1343spilt-KKH- | T | 0.02% | 0.02% | 0.01% | 0.02% | 0.01% | 0.01% | 0.01% | 99.81% |
| Cas9-A1343-C and | | | | | | | | | |
| dSpCas9-A1343-N | | | | | | | | | |
| nogRNA-A1343spilt-KKH- | C | 0.01% | 0.09% | 99.99% | 0.08% | 0.0% | 0.07% | 0.00% | 0.07% |
| Cas9-A1343-C and | | | | | | | | | |
| dSpCas9-A1343-N | | | | | | | | | |
| nogRNA-A1343spilt-KKH- | G | 99.97% | 0.05% | 0.00% | 0.03% | 0.02% | 0.04% | 99.98% | 0.04% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-G1322spilt-KKH- | A | 0.02% | 99.89% | 0.00% | 99.94% | 99.94% | 99.94% | 0.00% | 0.07% |
| Cas9-G1322-N and | | | | | | | | | |
| dSpCas9-G1322-C | | | | | | | | | |
| nogRNA-G1322spilt-KKH- | T | 0.00% | 0.01% | 0.01% | 0.01% | 0.01% | 0.00% | 0.00% | 99.83% |
| Cas9-G1322-N and | | | | | | | | | |
| dSpCas9-G1322-C | | | | | | | | | |
| nogRNA-G1322spilt-KKH- | C | 0.00% | 0.05% | 99.99% | 0.01% | 0.05% | 0.03% | 0.00% | 0.05% |
| Cas9-G1322-N and | | | | | | | | | |
| dSpCas9-G1322-C | | | | | | | | | |
| nogRNA-G1322spilt-KKH- | G | 99.98% | 0.05% | 0.00% | 0.04% | 0.01% | 0.02% | 100.00% | 0.04% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.00% | 0.00% | 0.00% | 0.00% | 0.00% | 0.01% | 0.00% | 0.00% |
| Cas9-G1322-N and | | | | | | | | | |
| dSpCas9-G1322-C | | | | | | | | | |
| nogRNA-G1322spilt-KKH- | A | 0.01% | 99.85% | 0.01% | 99.94% | 99.93% | 99.89% | 0.01% | 0.08% |
| Cas9-G1322-C and | | | | | | | | | |
| dSpCas9-G1322-N | | | | | | | | | |
| nogRNA-G1322spilt-KKH- | T | 0.00% | 0.02% | 0.01% | 0.01% | 0.01% | 0.01% | 0.00% | 99.82% |
| Cas9-G1322-C and | | | | | | | | | |
| dSpCas9-G1322-N | | | | | | | | | |
| nogRNA-G1322spilt-KKH- | C | 0.00% | 0.06% | 99.97% | 0.02% | 0.03% | 0.04% | 0.00% | 0.03% |
| Cas9-G1322-C and | | | | | | | | | |
| dSpCas9-G1322-N | | | | | | | | | |
| nogRNA-G1322spilt-KKH- | G | 99.98% | 0.06% | 0.00% | 0.03% | 0.03% | 0.05% | 99.99% | 0.07% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.00% | 0.01% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1333spilt-KKH- | A | 0.00% | 99.88% | 0.00% | 99.94% | 99.94% | 99.91% | 0.00% | 0.08% |
| Cas9-G1333-N and | | | | | | | | | |
| dSpCas9-G1333-C | | | | | | | | | |
| nogRNA-G1333spilt-KKH- | T | 0.00% | 0.01% | 0.03% | 0.00% | 0.01% | 0.00% | 0.01% | 99.81% |
| Cas9-G1333-N and | | | | | | | | | |
| dSpCas9-G1333-C | | | | | | | | | |
| nogRNA-G1333spilt-KKH- | C | 0.00% | 0.04% | 99.97% | 0.05% | 0.05% | 0.03% | 0.00% | 0.05% |
| Cas9-G1333-N and | | | | | | | | | |
| dSpCas9-G1333-C | | | | | | | | | |
| nogRNA-G1333spilt-KKH- | G | 100.00% | 0.06% | 0.00% | 0.00% | 0.00% | 0.06% | 99.99% | 0.06% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | A | 0.03% | 99.89% | 0.00% | 99.94% | 99.93% | 99.90% | 0.00% | 0.09% |
| Cas9-G1333-C and | | | | | | | | | |
| dSpCas9-G1333-N | | | | | | | | | |
| nogRNA-G1333spilt-KKH- | T | 0.00% | 0.00% | 0.00% | 0.01% | 0.01% | 0.02% | 0.01% | 99.80% |
| Cas9-G1333-C and | | | | | | | | | |
| dSpCas9-G1333-N | | | | | | | | | |
| nogRNA-G1333spilt-KKH- | C | 0.00% | 0.07% | 100.00% | 0.02% | 0.05% | 0.05% | 0.00% | 0.06% |
| Cas9-G1333-C and | | | | | | | | | |
| dSpCas9-G1333-N | | | | | | | | | |
| nogRNA-G1333spilt-KKH- | G | 99.96% | 0.03% | 0.00% | 0.03% | 0.01% | 0.03% | 99.99% | 0.05% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1371spilt-KKH- | A | 0.00% | 99.87% | 0.01% | 99.95% | 99.91% | 99.93% | 0.01% | 0.09% |
| Cas9-G1371-N and | | | | | | | | | |
| dSpCas9-G1371-C | | | | | | | | | |
| nogRNA-G1371spilt-KKH- | T | 0.02% | 0.03% | 0.02% | 0.02% | 0.00% | 0.00% | 0.01% | 99.79% |
| Cas9-G1371-N and | | | | | | | | | |
| dSpCas9-G1371-C | | | | | | | | | |
| nogRNA-G1371spilt-KKH- | C | 0.00% | 0.06% | 99.98% | 0.01% | 0.07% | 0.02% | 0.00% | 0.05% |
| Cas9-G1371-N and | | | | | | | | | |
| dSpCas9-G1371-C | | | | | | | | | |
| nogRNA-G1371spilt-KKH- | G | 99.98% | 0.03% | 0.00% | 0.02% | 0.02% | 0.05% | 99.98% | 0.07% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | A | 0.01% | 99.86% | 0.01% | 99.96% | 99.93% | 99.92% | 0.00% | 0.06% |
| Cas9-G1371-C and | | | | | | | | | |
| dSpCas9-G1371-N | | | | | | | | | |
| nogRNA-G1371spilt-KKH- | T | 0.01% | 0.02% | 0.02% | 0.01% | 0.01% | 0.01% | 0.01% | 99.85% |
| Cas9-G1371-C and | | | | | | | | | |
| dSpCas9-G1371-N | | | | | | | | | |
| nogRNA-G1371spilt-KKH- | C | 0.00% | 0.05% | 99.97% | 0.02% | 0.05% | 0.04% | 0.00% | 0.04% |
| Cas9-G1371-C and | | | | | | | | | |
| dSpCas9-G1371-N | | | | | | | | | |
| nogRNA-G1371spilt-KKH- | G | 99.98% | 0.07% | 0.00% | 0.02% | 0.01% | 0.03% | 99.99% | 0.05% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSnCas9-G1371-N | | | | | | | | | |
| nogRNA-G1371spilt-KKH- | — | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1397spilt-KKH- | A | 0.01% | 99.88% | 0.00% | 99.93% | 99.90% | 99.90% | 0.00% | 0.04% |
| Cas9-G1397-N and | | | | | | | | | |
| dSpCas9-G1397-C | | | | | | | | | |
| nogRNA-G1397spilt-KKH- | T | 0.00% | 0.01% | 0.02% | 0.01% | 0.02% | 0.00% | 0.00% | 99.88% |
| Cas9-G1397-N and | | | | | | | | | |
| dSpCas9-G1397-C | | | | | | | | | |
| nogRNA-G1397spilt-KKH- | C | 0.00% | 0.07% | 99.98% | 0.03% | 0.07% | 0.04% | 0.00% | 0.03% |
| Cas9-G1397-N and | | | | | | | | | |
| dSpCas9-G1397-C | | | | | | | | | |
| nogRNA-G1397spilt-KKH- | G | 99.99% | 0.04% | 0.00% | 0.02% | 0.01% | 0.05% | 99.99% | 0.05% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.00% | 0.00% | 0.00% | 0.00% | 0.00% | 0.01% | 0.00% | 0.00% |
| Cas9-G1397-N and | | | | | | | | | |
| dSpCas9-G1397-C | | | | | | | | | |
| nogRNA-G1397spilt-KKH- | A | 0.00% | 99.87% | 0.00% | 99.93% | 99.90% | 99.93% | 0.00% | 0.07% |
| Cas9-G1397-C and | | | | | | | | | |
| dSpCas9-G1397-N | | | | | | | | | |
| nogRNA-G1397spilt-KKH- | T | 0.00% | 0.01% | 0.04% | 0.01% | 0.00% | 0.00% | 0.00% | 99.83% |
| Cas9-G1397-C and | | | | | | | | | |
| dSpCas9-G1397-N | | | | | | | | | |
| nogRNA-G1397spilt-KKH- | C | 0.00% | 0.06% | 99.96% | 0.05% | 0.05% | 0.03% | 0.00% | 0.04% |
| Cas9-G1397-C and | | | | | | | | | |
| dSpCas9-G1397-N | | | | | | | | | |
| nogRNA-G1397spilt-KKH- | G | 99.99% | 0.06% | 0.00% | 0.01% | 0.04% | 0.03% | 99.99% | 0.06% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.00% | 0.01% | 0.00% | 0.00% | 0.00% | 0.01% | 0.01% | 0.00% |
| Cas9-G1397-C and | | | | | | | | | |
| dSpCas9-G1397-N | | | | | | | | | |
| nogRNA-N1357spilt-KKH- | A | 0.03% | 99.86% | 0.02% | 99.95% | 99.92% | 99.90% | 0.00% | 0.06% |
| Cas9-N1357-N and | | | | | | | | | |
| dSpCas9-N1357-C | | | | | | | | | |
| nogRNA-N1357spilt-KKH- | T | 0.00% | 0.03% | 0.01% | 0.00% | 0.01% | 0.01% | 0.00% | 99.83% |
| Cas9-N1357-N and | | | | | | | | | |
| dSpCas9-N1357-C | | | | | | | | | |
| nogRNA-N1357spilt-KKH- | C | 0.00% | 0.08% | 99.97% | 0.03% | 0.06% | 0.04% | 0.00% | 0.06% |
| Cas9-N1357-N and | | | | | | | | | |
| dSpCas9-N1357-C | | | | | | | | | |
| nogRNA-N1357spilt-KKH- | G | 99.97% | 0.03% | 0.00% | 0.02% | 0.01% | 0.03% | 100.00% | 0.04% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.00% | 0.00% | 0.00% | 0.00% | 0.00% | 0.01% | 0.00% | 0.00% |
| Cas9-N1357-N and | | | | | | | | | |
| dSpCas9-N1357-C | | | | | | | | | |
| nogRNA-N1357spilt-KKH- | A | 0.01% | 99.80% | 0.01% | 99.90% | 99.90% | 99.90% | 0.00% | 0.10% |
| Cas9-N1357-C and | | | | | | | | | |
| dSpCas9-N1357-N | | | | | | | | | |
| nogRNA-N1357spilt-KKH- | T | 0.04% | 0.03% | 0.01% | 0.00% | 0.01% | 0.01% | 0.01% | 99.80% |
| Cas9-N1357-C and | | | | | | | | | |
| dSpCas9-N1357-N | | | | | | | | | |
| nogRNA-N1357spilt-KKH- | C | 0.00% | 0.10% | 99.98% | 0.07% | 0.08% | 0.07% | 0.00% | 0.05% |
| Cas9-N1357-C and | | | | | | | | | |
| dSpCas9-N1357-N | | | | | | | | | |
| nogRNA-N1357spilt-KKH- | G | 99.95% | 0.07% | 0.00% | 0.03% | 0.01% | 0.02% | 99.99% | 0.05% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.00% | 0.00% | 0.00% | 0.00% | 0.00% | 0.01% | 0.00% | 0.00% |
| Cas9-N1357-C and | | | | | | | | | |
| dSpCas9-N1357-N | | | | | | | | | |
| nogRNA-N1387spilt-KKH- | A | 0.01% | 99.85% | 0.01% | 99.93% | 99.90% | 99.91% | 0.00% | 0.12% |
| Cas9-N1387-N and | | | | | | | | | |
| dSpCas9-N1387-C | | | | | | | | | |
| nogRNA-N1387spilt-KKH- | T | 0.01% | 0.02% | 0.01% | 0.00% | 0.03% | 0.01% | 0.00% | 99.81% |
| Cas9-N1387-N and | | | | | | | | | |
| dSpCas9-N1387-C | | | | | | | | | |
| nogRNA-N1387spilt-KKH- | C | 0.00% | 0.09% | 99.96 | 0.04% | 0.06% | 0.04% | 0.00% | 0.03% |
| Cas9-N1387-N and | | | | | | | | | |
| dSpCas9-N1387-C | | | | | | | | | |
| nogRNA-N1387spilt-KKH- | G | 99.97% | 0.03% | 0.00% | 0.02% | 0.01% | 0.04% | 99.99% | 0.04% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | A | 0.01% | 99.86% | 0.01% | 99.91% | 99.95% | 99.91% | 0.00% | 0.07% |
| Cas9-N1387-C and | | | | | | | | | |
| dSpCas9-N1387-N | | | | | | | | | |
| nogRNA-N1387spilt-KKH- | T | 0.01% | 0.03% | 0.01% | 0.02% | 0.01% | 0.00% | 0.00% | 99.86% |
| Cas9-N1387-C and | | | | | | | | | |
| dSpCas9-N1387-N | | | | | | | | | |
| nogRNA-N1387spilt-KKH- | C | 0.00% | 0.04% | 99.97% | 0.04% | 0.03% | 0.04% | 0.00% | 0.04% |
| Cas9-N1387-C and | | | | | | | | | |
| dSpCas9-N1387-N | | | | | | | | | |
| nogRNA-N1387spilt-KKH- | G | 99.98% | 0.07% | 0.00% | 0.04% | 0.00% | 0.04% | 99.99% | 0.02% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |

| Batch | Nucleotide | A | C | A | A | A | C | G |
|---|---|---|---|---|---|---|---|---|
| nogRNA-A1343spilt-KKH- | A | 99.94% | 0.03% | 99.98% | 99.99% | 99.96% | 0.01% | 0.01% |
| Cas9-A1343-N and | | | | | | | | |
| dSpCas9-A1343-C | | | | | | | | |
| nogRNA-A1343spilt-KKH- | T | 0.02% | 0.01% | 0.00% | 0.00% | 0.00% | 0.00% | 0.01% |
| Cas9-A1343-N and | | | | | | | | |
| dSpCas9-A1343-C | | | | | | | | |
| nogRNA-A1343spilt-KKH- | C | 0.01% | 99.96% | 0.00% | 0.00% | 0.02% | 99.97% | 0.02% |
| Cas9-A1343-N and | | | | | | | | |
| dSpCas9-A1343-C | | | | | | | | |
| nogRNA-A1343spilt-KKH- | G | 0.02% | 0.00% | 0.02% | 0.00% | 0.01% | 0.02% | 99.97% |
| Cas9-A1343-N and | | | | | | | | |
| dSpCas9-A1343-C | | | | | | | | |
| nogRNA-A1343spilt-KKH- | N | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | — | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | A | 99.88% | 0.05% | 99.91% | 99.93% | 99.92% | 0.02% | 0.02% |
| Cas9-A1343-C and | | | | | | | | |
| dSpCas9-A1343-N | | | | | | | | |
| nogRNA-A1343spilt-KKH- | T | 0.00% | 0.03% | 0.00% | 0.00% | 0.00% | 0.02% | 0.01% |
| Cas9-A1343-C and | | | | | | | | |
| dSpCas9-A1343-N | | | | | | | | |
| nogRNA-A1343spilt-KKH- | C | 0.09% | 99.92% | 0.06% | 0.05% | 0.08% | 99.95% | 0.00% |
| Cas9-A1343-C and | | | | | | | | |
| dSpCas9-A1343-N | | | | | | | | |
| nogRNA-A1343spilt-KKH- | G | 0.03% | 0.00% | 0.02% | 0.01% | 0.00% | 0.01% | 99.96% |
| Cas9-A1343-C and | | | | | | | | |
| dSpCas9-A1343-N | | | | | | | | |
| nogRNA-A1343spilt-KKH- | N | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-A1343spilt-KKH- | — | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-G1322spilt-KKH- | A | 99.95% | 0.03% | 99.96% | 99.97% | 99.97% | 0.02% | 0.01% |
| Cas9-G1322-N and | | | | | | | | |
| dSpCas9-G1322-C | | | | | | | | |
| nogRNA-G1322spilt-KKH- | T | 0.01% | 0.02% | 0.00% | 0.00% | 0.00% | 0.01% | 0.01% |
| Cas9-G1322-N and | | | | | | | | |
| dSpCas9-G1322-C | | | | | | | | |
| nogRNA-G1322spilt-KKH- | C | 0.01% | 99.94% | 0.01% | 0.02% | 0.02% | 99.96% | 0.00% |
| Cas9-G1322-N and | | | | | | | | |
| dSpCas9-G1322-C | | | | | | | | |
| nogRNA-G1322spilt-KKH- | G | 0.02% | 0.01% | 0.02% | 0.01% | 0.01% | 0.01% | 99.99% |
| Cas9-G1322-N and | | | | | | | | |
| dSpCas9-G1322-C | | | | | | | | |
| nogRNA-G1322spilt-KKH- | N | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | — | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | A | 99.97% | 0.01% | 99.97% | 99.97% | 99.97% | 0.02% | 0.01% |
| Cas9-G1322-C and | | | | | | | | |
| dSpCas9-G1322-N | | | | | | | | |
| nogRNA-G1322spilt-KKH- | T | 0.01% | 0.00% | 0.00% | 0.00% | 0.00% | 0.02% | 0.00% |
| Cas9-G1322-C and | | | | | | | | |
| dSpCas9-G1322-N | | | | | | | | |
| nogRNA-G1322spilt-KKH- | C | 0.01% | 99.99% | 0.02% | 0.02% | 0.02% | 99.95% | 0.00% |
| Cas9-G1322-C and | | | | | | | | |
| dSpCas9-G1322-N | | | | | | | | |
| nogRNA-G1322spilt-KKH- | G | 0.02% | 0.00% | 0.01% | 0.01% | 0.00% | 0.01% | 99.99% |
| Cas9-G1322-C and | | | | | | | | |
| dSpCas9-G1322-N | | | | | | | | |
| nogRNA-G1322spilt-KKH- | N | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1322spilt-KKH- | — | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1333spilt-KKH- | A | 99.95% | 0.03% | 99.97% | 99.98% | 100.00% | 0.03% | 0.04% |
| Cas9-G1333-N and | | | | | | | | |
| dSpCas9-G1333-C | | | | | | | | |
| nogRNA-G1333spilt-KKH- | T | 0.01% | 0.01% | 0.00% | 0.00% | 0.00% | 0.04% | 0.01% |
| Cas9-G1333-N and | | | | | | | | |
| dSpCas9-G1333-C | | | | | | | | |
| nogRNA-G1333spilt-KKH- | C | 0.02% | 99.96% | 0.01% | 0.00% | 0.00% | 99.92% | 0.00% |
| Cas9-G1333-N and | | | | | | | | |
| dSpCas9-G1333-C | | | | | | | | |
| nogRNA-G1333spilt-KKH- | G | 0.02% | 0.00% | 0.02% | 0.02% | 0.00% | 0.01% | 99.94% |
| Cas9-G1333-N and | | | | | | | | |
| dSpCas9-G1333-C | | | | | | | | |
| nogRNA-G1333spilt-KKH- | N | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | — | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | A | 99.98% | 0.00% | 99.96% | 99.96% | 99.97% | 0.01% | 0.01% |
| Cas9-G1333-C and | | | | | | | | |
| dSpCas9-G1333-N | | | | | | | | |
| nogRNA-G1333spilt-KKH- | T | 0.01% | 0.01% | 0.00% | 0.00% | 0.00% | 0.03% | 0.00% |
| Cas9-G1333-C and | | | | | | | | |
| dSpCas9-G1333-N | | | | | | | | |
| nogRNA-G1333spilt-KKH- | C | 0.00% | 99.97% | 0.02% | 0.02% | 0.01% | 99.96% | 0.00% |
| Cas9-G1333-C and | | | | | | | | |
| dSpCas9-G1333-N | | | | | | | | |
| nogRNA-G1333spilt-KKH- | G | 0.00% | 0.01% | 0.01% | 0.02% | 0.01% | 0.00% | 99.98% |
| Cas9-G1333-C and | | | | | | | | |
| dSpCas9-G1333-N | | | | | | | | |
| nogRNA-G1333spilt-KKH- | N | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1333spilt-KKH- | — | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1371spilt-KKH- | A | 99.97% | 0.02% | 99.97% | 99.97% | 99.95% | 0.02% | 0.02% |
| Cas9-G1371-N and | | | | | | | | |
| dSpCas9-G1371-C | | | | | | | | |
| nogRNA-G1371spilt-KKH- | T | 0.01% | 0.01% | 0.00% | 0.00% | 0.00% | 0.02% | 0.00% |
| Cas9-G1371-N and | | | | | | | | |
| dSpCas9-G1371-C | | | | | | | | |
| nogRNA-G1371spilt-KKH- | C | 0.01% | 99.97% | 0.02% | 0.02% | 0.03% | 99.96% | 0.01% |
| Cas9-G1371-N and | | | | | | | | |
| dSpCas9-G1371-C | | | | | | | | |
| nogRNA-G1371spilt-KKH- | G | 0.01% | 0.00% | 0.01% | 0.02% | 0.01% | 0.00% | 99.97% |
| Cas9-G1371-N and | | | | | | | | |
| dSpCas9-G1371-C | | | | | | | | |
| nogRNA-G1371spilt-KKH- | N | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | — | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | A | 99.97% | 0.02% | 99.95% | 99.98% | 99.96% | 0.01% | 0.02% |
| Cas9-G1371-C and | | | | | | | | |
| dSpCas9-G1371-N | | | | | | | | |
| nogRNA-G1371spilt-KKH- | T | 0.01% | 0.01% | 0.00% | 0.01% | 0.00% | 0.02% | 0.01% |
| Cas9-G1371-C and | | | | | | | | |
| dSpCas9-G1371-N | | | | | | | | |
| nogRNA-G1371spilt-KKH- | C | 0.01% | 99.96% | 0.02% | 0.01% | 0.03% | 99.95% | 0.00% |
| Cas9-G1371-C and | | | | | | | | |
| dSpCas9-G1371-N | | | | | | | | |
| nogRNA-G1371spilt-KKH- | G | 0.01% | 0.00% | 0.02% | 0.01% | 0.00% | 0.01% | 99.97% |
| Cas9-G1371-C and | | | | | | | | |
| dSpCas9-G1371-N | | | | | | | | |
| nogRNA-G1371spilt-KKH- | N | 0.00% | 0.00% | 0.00% | 0.00% | 0.00% | 0.00% | 0.00% |
| Cas9-G1371-C and | | | | | | | | |
| dSnCas9-G1371-N | | | | | | | | |
| nogRNA-G1371spilt-KKH- | — | 0.00% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1397spilt-KKH- | A | 99.95% | 0.00% | 99.99% | 99.97% | 99.95% | 0.02% | 0.02% |
| Cas9-G1397-N and | | | | | | | | |
| dSpCas9-G1397-C | | | | | | | | |
| nogRNA-G1397spilt-KKH- | T | 0.01% | 0.01% | 0.00% | 0.00% | 0.00% | 0.02% | 0.00% |
| Cas9-G1397-N and | | | | | | | | |
| dSpCas9-G1397-C | | | | | | | | |
| nogRNA-G1397spilt-KKH- | C | 0.02% | 99.99% | 0.00% | 0.00% | 0.02% | 99.95% | 0.00% |
| Cas9-G1397-N and | | | | | | | | |
| dSpCas9-G1397-C | | | | | | | | |
| nogRNA-G1397spilt-KKH- | G | 0.01% | 0.00% | 0.01% | 0.02% | 0.01% | 0.01% | 99.97% |
| Cas9-G1397-N and | | | | | | | | |
| dSpCas9-G1397-C | | | | | | | | |
| nogRNA-G1397spilt-KKH- | N | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | — | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | A | 99.96% | 0.02% | 99.98% | 99.93% | 99.97% | 0.01% | 0.02% |
| Cas9-G1397-C and | | | | | | | | |
| dSpCas9-G1397-N | | | | | | | | |
| nogRNA-G1397spilt-KKH- | T | 0.01% | 0.01% | 0.00% | 0.01% | 0.00% | 0.03% | 0.00% |
| Cas9-G1397-C and | | | | | | | | |
| dSpCas9-G1397-N | | | | | | | | |
| nogRNA-G1397spilt-KKH- | C | 0.00% | 99.96% | 0.01% | 0.03% | 0.02% | 99.95% | 0.00% |
| Cas9-G1397-C and | | | | | | | | |
| dSpCas9-G1397-N | | | | | | | | |
| nogRNA-G1397spilt-KKH- | G | 0.03% | 0.01% | 0.01% | 0.03% | 0.01% | 0.00% | 99.98% |
| Cas9-G1397-C and | | | | | | | | |
| dSpCas9-G1397-N | | | | | | | | |
| nogRNA-G1397spilt-KKH- | N | 0.00% | 0.00% | 0.00% | 0.00% | 0.00% | 0.00% | 0.00% |
| Cas9-G1397-C and | | | | | | | | |
| dSpCas9-G1397-N | | | | | | | | |
| nogRNA-G1397spilt-KKH- | — | 0.00% | 0.00% | 0.00% | 0.00% | 0.00% | 0.01% | 0.00% |
| Cas9-G1397-C and | | | | | | | | |
| dSpCas9-G1397-N | | | | | | | | |
| nogRNA-N1357spilt-KKH- | A | 99.94% | 0.00% | 99.99% | 99.98% | 99.98% | 0.00% | 0.01% |
| Cas9-N1357-N and | | | | | | | | |
| dSpCas9-N1357-C | | | | | | | | |
| nogRNA-N1357spilt-KKH- | T | 0.02% | 0.01% | 0.00% | 0.01% | 0.00% | 0.01% | 0.00% |
| Cas9-N1357-N and | | | | | | | | |
| dSpCas9-N1357-C | | | | | | | | |
| nogRNA-N1357spilt-KKH- | C | 0.02% | 99.98% | 0.00% | 0.00% | 0.02% | 99.98% | 0.00% |
| Cas9-N1357-N and | | | | | | | | |
| dSpCas9-N1357-C | | | | | | | | |
| nogRNA-N1357spilt-KKH- | G | 0.03% | 0.00% | 0.00% | 0.01% | 0.00% | 0.00% | 99.99% |
| Cas9-N1357-N and | | | | | | | | |
| dSpCas9-N1357-C | | | | | | | | |
| nogRNA-N1357spilt-KKH- | N | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | — | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | A | 99.93% | 0.04% | 99.94% | 99.92% | 99.93% | 0.01% | 0.02% |
| Cas9-N1357-C and | | | | | | | | |
| dSpCas9-N1357-N | | | | | | | | |
| nogRNA-N1357spilt-KKH- | T | 0.00% | 0.02% | 0.00% | 0.00% | 0.00% | 0.02% | 0.00% |
| Cas9-N1357-C and | | | | | | | | |
| dSpCas9-N1357-N | | | | | | | | |
| nogRNA-N1357spilt-KKH- | C | 0.05% | 99.94% | 0.04% | 0.05% | 0.05% | 99.96% | 0.01% |
| Cas9-N1357-C and | | | | | | | | |
| dSpCas9-N1357-N | | | | | | | | |
| nogRNA-N1357spilt-KKH- | G | 0.02% | 0.00% | 0.01% | 0.03% | 0.02% | 0.02% | 99.97% |
| Cas9-N1357-C and | | | | | | | | |
| dSpCas9-N1357-N | | | | | | | | |
| nogRNA-N1357spilt-KKH- | N | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1357spilt-KKH- | — | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1387spilt-KKH- | A | 99.96% | 0.03% | 99.97% | 99.96% | 99.98% | 0.01% | 0.02% |
| Cas9-N1387-N and | | | | | | | | |
| dSpCas9-N1387-C | | | | | | | | |
| nogRNA-N1387spilt-KKH- | T | 0.00% | 0.00% | 0.00% | 0.00% | 0.00% | 0.01% | 0.00% |
| Cas9-N1387-N and | | | | | | | | |
| dSpCas9-N1387-C | | | | | | | | |
| nogRNA-N1387spilt-KKH- | C | 0.02% | 99.96% | 0.01% | 0.01% | 0.01% | 99.97% | 0.00% |
| Cas9-N1387-N and | | | | | | | | |
| dSpCas9-N1387-C | | | | | | | | |
| nogRNA-N1387spilt-KKH- | G | 0.01% | 0.00% | 0.01% | 0.02% | 0.00% | 0.01% | 99.97% |
| Cas9-N1387-N and | | | | | | | | |
| dSpCas9-N1387-C | | | | | | | | |
| nogRNA-N1387spilt-KKH- | N | 0.00% | 0.00% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | — | 0.00% | 0.01% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | A | 99.96% | 0.02% | 99.98% | 99.98% | 99.97% | 0.01% | 0.01% |
| Cas9-N1387-C and | | | | | | | | |
| dSpCas9-N1387-N | | | | | | | | |
| nogRNA-N1387spilt-KKH- | T | 0.01% | 0.02% | 0.00% | 0.00% | 0.00% | 0.04% | 0.00% |
| Cas9-N1387-C and | | | | | | | | |
| dSpCas9-N1387-N | | | | | | | | |
| nogRNA-N1387spilt-KKH- | C | 0.00% | 99.96% | 0.01% | 0.01% | 0.02% | 99.95% | 0.01% |
| Cas9-N1387-C and | | | | | | | | |
| dSpCas9-N1387-N | | | | | | | | |
| nogRNA-N1387spilt-KKH- | G | 0.02% | 0.00% | 0.00% | 0.00% | 0.01% | 0.01% | 99.98% |
| Cas9-N1387-C and | | | | | | | | |
| dSpCas9-N1387-N | | | | | | | | |
| nogRNA-N1387spilt-KKH- | N | 0.00% | 0.00% | 0.00% | 0.00% | 0.00% | 0.00% | 0.00% |
| Cas9-N1387-C and | | | | | | | | |
| dSpCas9-N1387-N | | | | | | | | |
| nogRNA-N1387spilt-KKH- | — | 0.00% | 0.00% | 0.00% | 0.00% | 0.00% | 0.00% | 0.00% |
| Cas9-N1387-C and | | | | | | | | |
| dSpCas9-N1387-N | | | | | | | | |

| Batch | Nucleotide | G | C | A | G | A | A | G | C | T |
|---|---|---|---|---|---|---|---|---|---|---|
| nogRNA-A1343spilt-KKH- | A | 0.01% | 0.01% | 99.93% | 0.00% | 99.94% | 99.92% | 0.00% | 0.00% | 0.00% |
| Cas9-A1343-N and | | | | | | | | | | |
| dSpCas9-A1343-C | | | | | | | | | | |
| nogRNA-A1343spilt-KKH- | T | 0.02% | 0.02% | 0.00% | 0.00% | 0.01% | 0.00% | 0.00% | 0.01% | 99.95% |
| Cas9-A1343-N and | | | | | | | | | | |
| dSpCas9-A1343-C | | | | | | | | | | |
| nogRNA-A1343spilt-KKH- | C | 0.00% | 99.96% | 0.00% | 0.00% | 0.01% | 0.02% | 0.00% | 99.98% | 0.02% |
| Cas9-A1343-N and | | | | | | | | | | |
| dSpCas9-A1343-C | | | | | | | | | | |
| nogRNA-A1343spilt-KKH- | G | 99.98% | 0.01% | 0.07% | 99.99% | 0.04% | 0.06% | 99.98% | 0.00% | 0.02% |
| Cas9-A1343-N and | | | | | | | | | | |
| dSpCas9-A1343-C | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-N and | | | | | | | | | | |
| dSpCas9-A1343-C | | | | | | | | | | |
| nogRNA-A1343spilt-KKH- | A | 0.02% | 0.02% | 99.82% | 0.01% | 99.91% | 99.83% | 0.00% | 0.00% | 0.00% |
| Cas9-A1343-C and | | | | | | | | | | |
| dSpCas9-A1343-N | | | | | | | | | | |
| nogRNA-A1343spilt-KKH- | T | 0.00% | 0.01% | 0.01% | 0.00% | 0.00% | 0.01% | 0.00% | 0.00% | 99.94% |
| Cas9-A1343-C and | | | | | | | | | | |
| dSpCas9-A1343-N | | | | | | | | | | |
| nogRNA-A1343spilt-KKH- | C | 0.00% | 99.94% | 0.08% | 0.00% | 0.07% | 0.08% | 0.00% | 99.98% | 0.01% |
| Cas9-A1343-C and | | | | | | | | | | |
| dSpCas9-A1343-N | | | | | | | | | | |
| nogRNA-A1343spilt-KKH- | G | 99.97% | 0.03% | 0.09% | 99.98% | 0.02% | 0.08% | 99.99% | 0.01% | 0.05% |
| Cas9-A1343-C and | | | | | | | | | | |
| dSpCas9-A1343-N | | | | | | | | | | |
| nogRNA-A1343spilt-KKH- | N | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-A1343spilt-KKH- | — | 0.00% | 0.00% | 0.00% | 0.00% | 0.00% | 0.00% | 0.00% | 0.00% | 0.00% |
| Cas9-A1343-C and | | | | | | | | | | |
| dSpCas9-A1343-N | | | | | | | | | | |
| nogRNA-G1322spilt-KKH- | A | 0.03% | 0.01% | 99.93% | 0.01% | 99.96% | 99.93% | 0.00% | 0.00% | 0.00% |
| Cas9-G1322-N and | | | | | | | | | | |
| dSpCas9-G1322-C | | | | | | | | | | |
| nogRNA-G1322spilt-KKH- | T | 0.01% | 0.01% | 0.00% | 0.01% | 0.00% | 0.01% | 0.00% | 0.01% | 99.96% |
| Cas9-G1322-N and | | | | | | | | | | |
| dSpCas9-G1322-C | | | | | | | | | | |
| nogRNA-G1322spilt-KKH- | C | 0.01% | 99.96% | 0.01% | 0.00% | 0.03% | 0.02% | 0.00% | 99.98% | 0.02% |
| Cas9-G1322-N and | | | | | | | | | | |
| dSpCas9-G1322-C | | | | | | | | | | |
| nogRNA-G1322spilt-KKH- | G | 99.95% | 0.02% | 0.05% | 99.98% | 0.01% | 0.05% | 100.00% | 0.01% | 0.02% |
| Cas9-G1322-N and | | | | | | | | | | |
| dSpCas9-G1322-C | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-N and | | | | | | | | | | |
| dSpCas9-G1322-C | | | | | | | | | | |
| nogRNA-G1322spilt-KKH- | A | 0.02% | 0.01% | 99.87% | 0.02% | 99.95% | 99.89% | 0.00% | 0.00% | 0.00% |
| Cas9-G1322-C and | | | | | | | | | | |
| dSpCas9-G1322-N | | | | | | | | | | |
| nogRNA-G1322spilt-KKH- | T | 0.00% | 0.01% | 0.01% | 0.00% | 0.00% | 0.00% | 0.00% | 0.03% | 99.95% |
| Cas9-G1322-C and | | | | | | | | | | |
| dSpCas9-G1322-N | | | | | | | | | | |
| nogRNA-G1322spilt-KKH- | C | 0.00% | 99.96% | 0.03% | 0.00% | 0.01% | 0.02% | 0.00% | 99.96% | 0.02% |
| Cas9-G1322-C and | | | | | | | | | | |
| dSpCas9-G1322-N | | | | | | | | | | |
| nogRNA-G1322spilt-KKH- | G | 99.97% | 0.02% | 0.08% | 99.98% | 0.03% | 0.09% | 99.99% | 0.02% | 0.03% |
| Cas9-G1322-C and | | | | | | | | | | |
| dSpCas9-G1322-N | | | | | | | | | | |
| nogRNA-G1322spilt-KKH- | N | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1322spilt-KKH- | — | 0.00% | 0.00% | 0.00% | 0.00% | 0.00% | 0.00% | 0.00% | 0.00% | 0.00% |
| Cas9-G1322-C and | | | | | | | | | | |
| dSpCas9-G1322-N | | | | | | | | | | |
| nogRNA-G1333spilt-KKH- | A | 0.02% | 0.00% | 99.94% | 0.01% | 99.96% | 99.85% | 0.00% | 0.00% | 0.00% |
| Cas9-G1333-N and | | | | | | | | | | |
| dSpCas9-G1333-C | | | | | | | | | | |
| nogRNA-G1333spilt-KKH- | T | 0.01% | 0.02% | 0.00% | 0.00% | 0.00% | 0.00% | 0.01% | 0.01% | 99.97% |
| Cas9-G1333-N and | | | | | | | | | | |
| dSpCas9-G1333-C | | | | | | | | | | |
| nogRNA-G1333spilt-KKH- | C | 0.00% | 99.96% | 0.01% | 0.00% | 0.02% | 0.05% | 0.01% | 99.98% | 0.01% |
| Cas9-G1333-N and | | | | | | | | | | |
| dSpCas9-G1333-C | | | | | | | | | | |
| nogRNA-G1333spilt-KKH- | G | 99.96% | 0.02% | 0.04% | 99.99% | 0.02% | 0.10% | 99.98% | 0.00% | 0.02% |
| Cas9-G1333-N and | | | | | | | | | | |
| dSpCas9-G1333-C | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-N and | | | | | | | | | | |
| dSpCas9-G1333-C | | | | | | | | | | |
| nogRNA-G1333spilt-KKH- | A | 0.03% | 0.01% | 99.93% | 0.00% | 99.95% | 99.88% | 0.00% | 0.00% | 0.00% |
| Cas9-G1333-C and | | | | | | | | | | |
| dSpCas9-G1333-N | | | | | | | | | | |
| nogRNA-G1333spilt-KKH- | T | 0.01% | 0.01% | 0.00% | 0.01% | 0.00% | 0.00% | 0.00% | 0.01% | 99.94% |
| Cas9-G1333-C and | | | | | | | | | | |
| dSpCas9-G1333-N | | | | | | | | | | |
| nogRNA-G1333spilt-KKH- | C | 0.01% | 99.95% | 0.01% | 0.00% | 0.03% | 0.03% | 0.01% | 99.99% | 0.04% |
| Cas9-G1333-C and | | | | | | | | | | |
| dSpCas9-G1333-N | | | | | | | | | | |
| nogRNA-G1333spilt-KKH- | G | 99.95% | 0.01% | 0.05% | 99.99% | 0.02% | 0.09% | 99.99% | 0.00% | 0.03% |
| Cas9-G1333-C and | | | | | | | | | | |
| dSpCas9-G1333-N | | | | | | | | | | |
| nogRNA-G1333spilt-KKH- | N | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1333spilt-KKH- | — | 0.00% | 0.00% | 0.00% | 0.00% | 0.00% | 0.00% | 0.00% | 0.00% | 0.00% |
| Cas9-G1333-C and | | | | | | | | | | |
| dSpCas9-G1333-N | | | | | | | | | | |
| nogRNA-G1371spilt-KKH- | A | 0.04% | 0.02% | 99.92% | 0.01% | 99.96% | 99.90% | 0.01% | 0.01% | 0.00% |
| Cas9-G1371-N and | | | | | | | | | | |
| dSpCas9-G1371-C | | | | | | | | | | |
| nogRNA-G1371spilt-KKH- | T | 0.00% | 0.00% | 0.00% | 0.00% | 0.01% | 0.00% | 0.00% | 0.01% | 99.94% |
| Cas9-G1371-N and | | | | | | | | | | |
| dSpCas9-G1371-C | | | | | | | | | | |
| nogRNA-G1371spilt-KKH- | C | 0.00% | 99.96% | 0.01% | 0.00% | 0.03% | 0.01% | 0.00% | 99.98% | 0.03% |
| Cas9-G1371-N and | | | | | | | | | | |
| dSpCas9-G1371-C | | | | | | | | | | |
| nogRNA-G1371spilt-KKH- | G | 99.95% | 0.01% | 0.06% | 99.98% | 0.01% | 0.08% | 99.99% | 0.00% | 0.03% |
| Cas9-G1371-N and | | | | | | | | | | |
| dSpCas9-G1371-C | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | — | 0.00% | 0.01% | 0.00% | 0.01% | 0.00% | 0.00% | 0.00% | 0.00% | 0.00% |
| Cas9-G1371-N and | | | | | | | | | | |
| dSpCas9-G1371-C | | | | | | | | | | |
| nogRNA-G1371spilt-KKH- | A | 0.02% | 0.01% | 99.92% | 0.01% | 99.95% | 99.93% | 0.00% | 0.00% | 0.00% |
| Cas9-G1371-C and | | | | | | | | | | |
| dSpCas9-G1371-N | | | | | | | | | | |
| nogRNA-G1371spilt-KKH- | T | 0.01% | 0.00% | 0.01% | 0.00% | 0.00% | 0.00% | 0.01% | 0.01% | 99.94% |
| Cas9-G1371-C and | | | | | | | | | | |
| dSpCas9-G1371-N | | | | | | | | | | |
| nogRNA-G1371spilt-KKH- | C | 0.00% | 99.98% | 0.01% | 0.00% | 0.03% | 0.01% | 0.00% | 99.98% | 0.03% |
| Cas9-G1371-C and | | | | | | | | | | |
| dSpCas9-G1371-N | | | | | | | | | | |
| nogRNA-G1371spilt-KKH- | G | 99.96% | 0.01% | 0.05% | 99.99% | 0.02% | 0.05% | 99.98% | 0.01% | 0.02% |
| Cas9-G1371-C and | | | | | | | | | | |
| dSpCas9-G1371-N | | | | | | | | | | |
| nogRNA-G1371spilt-KKH- | N | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1371spilt-KKH- | — | 0.00% | 0.00% | 0.00% | 0.00% | 0.00% | 0.00% | 0.00% | 0.00% | 0.00% |
| Cas9-G1371-C and | | | | | | | | | | |
| dSpCas9-G1371-N | | | | | | | | | | |
| nogRNA-G1397spilt-KKH- | A | 0.04% | 0.02% | 99.92% | 0.01% | 99.95% | 99.89% | 0.00% | 0.00% | 0.01% |
| Cas9-G1397-N and | | | | | | | | | | |
| dSpCas9-G1397-C | | | | | | | | | | |
| nogRNA-G1397spilt-KKH- | T | 0.00% | 0.02% | 0.00% | 0.01% | 0.00% | 0.00% | 0.00% | 0.02% | 99.95% |
| Cas9-G1397-N and | | | | | | | | | | |
| dSpCas9-G1397-C | | | | | | | | | | |
| nogRNA-G1397spilt-KKH- | C | 0.00% | 99.95% | 0.02% | 0.00% | 0.03% | 0.02% | 0.00% | 99.97% | 0.01% |
| Cas9-G1397-N and | | | | | | | | | | |
| dSpCas9-G1397-C | | | | | | | | | | |
| nogRNA-G1397spilt-KKH- | G | 99.96% | 0.01% | 0.06% | 99.98% | 0.02% | 0.09% | 99.99% | 0.01% | 0.03% |
| Cas9-G1397-N and | | | | | | | | | | |
| dSpCas9-G1397-C | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | — | 0.00% | 0.00% | 0.00% | 0.00% | 0.00% | 0.00% | 0.00% | 0.00% | 0.00% |
| Cas9-G1397-N and | | | | | | | | | | |
| dSpCas9-G1397-C | | | | | | | | | | |
| nogRNA-G1397spilt-KKH- | A | 0.05% | 0.00% | 99.95% | 0.00% | 99.95% | 99.91% | 0.00% | 0.01% | 0.00% |
| Cas9-G1397-C and | | | | | | | | | | |
| dSpCas9-G1397-N | | | | | | | | | | |
| nogRNA-G1397spilt-KKH- | T | 0.01% | 0.01% | 0.00% | 0.00% | 0.00% | 0.00% | 0.00% | 0.02% | 99.96% |
| Cas9-G1397-C and | | | | | | | | | | |
| dSpCas9-G1397-N | | | | | | | | | | |
| nogRNA-G1397spilt-KKH- | C | 0.00% | 99.97% | 0.00% | 0.00% | 0.04% | 0.02% | 0.00% | 99.96% | 0.01% |
| Cas9-G1397-C and | | | | | | | | | | |
| dSpCas9-G1397-N | | | | | | | | | | |
| nogRNA-G1397spilt-KKH- | G | 99.94% | 0.01% | 0.04% | 100.00% | 0.01% | 0.07% | 100.00% | 0.01% | 0.03% |
| Cas9-G1397-C and | | | | | | | | | | |
| dSpCas9-G1397-N | | | | | | | | | | |
| nogRNA-G1397spilt-KKH- | N | 0.00% | 0.00% | 0.00% | 0.00% | 0.00% | 0.00% | 0.00% | 0.00% | 0.00% |
| Cas9-G1397-C and | | | | | | | | | | |
| dSpCas9-G1397-N | | | | | | | | | | |
| nogRNA-G1397spilt-KKH- | — | 0.00% | 0.00% | 0.00% | 0.00% | 0.00% | 0.01% | 0.00% | 0.00% | 0.00% |
| Cas9-G1397-C and | | | | | | | | | | |
| dSpCas9-G1397-N | | | | | | | | | | |
| nogRNA-N1357spilt-KKH- | A | 0.05% | 0.00% | 99.93% | 0.00% | 99.96% | 99.93% | 0.01% | 0.00% | 0.00% |
| Cas9-N1357-N and | | | | | | | | | | |
| dSpCas9-N1357-C | | | | | | | | | | |
| nogRNA-N1357spilt-KKH- | T | 0.00% | 0.00% | 0.00% | 0.01% | 0.01% | 0.00% | 0.00% | 0.02% | 99.97% |
| Cas9-N1357-N and | | | | | | | | | | |
| dSpCas9-N1357-C | | | | | | | | | | |
| nogRNA-N1357spilt-KKH- | C | 0.00% | 99.98% | 0.01% | 0.00% | 0.01% | 0.00% | 0.00% | 99.97% | 0.02% |
| Cas9-N1357-N and | | | | | | | | | | |
| dSpCas9-N1357-C | | | | | | | | | | |
| nogRNA-N1357spilt-KKH- | G | 99.95% | 0.01% | 0.06% | 99.99% | 0.02% | 0.07% | 99.98% | 0.01% | 0.00% |
| Cas9-N1357-N and | | | | | | | | | | |
| dSpCas9-N1357-C | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-N and | | | | | | | | | | |
| dSpCas9-N1357-C | | | | | | | | | | |
| nogRNA-N1357spilt-KKH- | A | 0.05% | 0.02% | 99.84% | 0.01% | 99.90% | 99.82% | 0.02% | 0.00% | 0.01% |
| Cas9-N1357-C and | | | | | | | | | | |
| dSpCas9-N1357-N | | | | | | | | | | |
| nogRNA-N1357spilt-KKH- | T | 0.00% | 0.02% | 0.02% | 0.00% | 0.01% | 0.00% | 0.00% | 0.01% | 99.97% |
| Cas9-N1357-C and | | | | | | | | | | |
| dSpCas9-N1357-N | | | | | | | | | | |
| nogRNA-N1357spilt-KKH- | C | 0.01% | 99.96% | 0.08% | 0.00% | 0.06% | 0.07% | 0.00% | 99.98% | 0.01% |
| Cas9-N1357-C and | | | | | | | | | | |
| dSpCas9-N1357-N | | | | | | | | | | |
| nogRNA-N1357spilt-KKH- | G | 99.94% | 0.01% | 0.06% | 99.99% | 0.04% | 0.11% | 99.98% | 0.02% | 0.02% |
| Cas9-N1357-C and | | | | | | | | | | |
| dSpCas9-N1357-N | | | | | | | | | | |
| nogRNA-N1357spilt-KKH- | N | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1357spilt-KKH- | — | 0.00% | 0.00% | 0.00% | 0.00% | 0.00% | 0.00% | 0.00% | 0.00% | 0.00% |
| Cas9-N1357-C and | | | | | | | | | | |
| dSpCas9-N1357-N | | | | | | | | | | |
| nogRNA-N1387spilt-KKH- | A | 0.03% | 0.02% | 99.93% | 0.01% | 99.94% | 99.88% | 0.01% | 0.00% | 0.01% |
| Cas9-N1387-N and | | | | | | | | | | |
| dSpCas9-N1387-C | | | | | | | | | | |
| nogRNA-N1387spilt-KKH- | T | 0.01% | 0.02% | 0.00% | 0.00% | 0.00% | 0.00% | 0.00% | 0.03% | 99.92% |
| Cas9-N1387-N and | | | | | | | | | | |
| dSpCas9-N1387-C | | | | | | | | | | |
| nogRNA-N1387spilt-KKH- | C | 0.01% | 99.96% | 0.01% | 0.00% | 0.03% | 0.04% | 0.00% | 99.96% | 0.02% |
| Cas9-N1387-N and | | | | | | | | | | |
| dSpCas9-N1387-C | | | | | | | | | | |
| nogRNA-N1387spilt-KKH- | G | 99.96% | 0.00% | 0.05% | 99.99% | 0.03% | 0.08% | 99.99% | 0.00% | 0.05% |
| Cas9-N1387-N and | | | | | | | | | | |
| dSpCas9-N1387-C | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | — | 0.00% | 0.00% | 0.00% | 0.00% | 0.00% | 0.00% | 0.00% | 0.00% | 0.00% |
| Cas9-N1387-N and | | | | | | | | | | |
| dSpCas9-N1387-C | | | | | | | | | | |
| nogRNA-N1387spilt-KKH- | A | 0.02% | 0.01% | 99.90% | 0.01% | 99.94% | 99.89% | 0.01% | 0.00% | 0.00% |
| Cas9-N1387-C and | | | | | | | | | | |
| dSpCas9-N1387-N | | | | | | | | | | |
| nogRNA-N1387spilt-KKH- | T | 0.00% | 0.00% | 0.01% | 0.00% | 0.00% | 0.00% | 0.01% | 0.01% | 99.95% |
| Cas9-N1387-C and | | | | | | | | | | |
| dSpCas9-N1387-N | | | | | | | | | | |
| nogRNA-N1387spilt-KKH- | C | 0.00% | 99.96% | 0.03% | 0.00% | 0.04% | 0.02% | 0.00% | 99.98% | 0.02% |
| Cas9-N1387-C and | | | | | | | | | | |
| dSpCas9-N1387-N | | | | | | | | | | |
| nogRNA-N1387spilt-KKH- | G | 99.97% | 0.02% | 0.05% | 99.99% | 0.02% | 0.09% | 99.97% | 0.01% | 0.03% |
| Cas9-N1387-C and | | | | | | | | | | |
| dSpCas9-N1387-N | | | | | | | | | | |
| nogRNA-N1387spilt-KKH- | N | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |
| nogRNA-N1387spilt-KKH- | — | 0.00% | 0.00% | 0.00% | 0.00% | 0.00% | 0.00% | 0.00% | 0.00% | 0.00% |
| Cas9-N1387-C and | | | | | | | | | | |
| dSpCas9-N1387-N | | | | | | | | | | |

| Batch | Nucleotide | G | G | A | G | G | A | G | G |
|---|---|---|---|---|---|---|---|---|---|
| nogRNA-A1343spilt-KKH- | A | 0.00% | 0.00% | 99.83% | 0.00% | 0.01% | 99.85% | 0.02% | 0.00% |
| Cas9-A1343-N and | | | | | | | | | |
| dSpCas9-A1343-C | | | | | | | | | |
| nogRNA-A1343spilt-KKH- | T | 0.01% | 0.01% | 0.00% | 0.01% | 0.02% | 0.02% | 0.01% | 0.01% |
| Cas9-A1343-N and | | | | | | | | | |
| dSpCas9-A1343-C | | | | | | | | | |
| nogRNA-A1343spilt-KKH- | C | 0.00% | 0.00% | 0.03% | 0.00% | 0.00% | 0.04% | 0.00% | 0.00% |
| Cas9-A1343-N and | | | | | | | | | |
| dSpCas9-A1343-C | | | | | | | | | |
| nogRNA-A1343spilt-KKH- | G | 99.98% | 99.98% | 0.14% | 99.99% | 99.97% | 0.10% | 99.97% | 99.99% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | A | 0.00% | 0.01% | 99.66% | 0.00% | 0.00% | 99.75% | 0.00% | 0.01% |
| Cas9-A1343-C and | | | | | | | | | |
| dSpCas9-A1343-N | | | | | | | | | |
| nogRNA-A1343spilt-KKH- | T | 0.00% | 0.00% | 0.02% | 0.02% | 0.02% | 0.01% | 0.01% | 0.00% |
| Cas9-A1343-C and | | | | | | | | | |
| dSpCas9-A1343-N | | | | | | | | | |
| nogRNA-A1343spilt-KKH- | C | 0.00% | 0.01% | 0.09% | 0.00% | 0.00% | 0.09% | 0.01% | 0.00% |
| Cas9-A1343-C and | | | | | | | | | |
| dSpCas9-A1343-N | | | | | | | | | |
| nogRNA-A1343spilt-KKH- | G | 99.99% | 99.98% | 0.23% | 99.98% | 99.98% | 0.15% | 99.97% | 99.98% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.00% | 0.00% | 0.00% | 0.00% | 0.00% | 0.00% | 0.01% | 0.01% |
| Cas9-A1343-C and | | | | | | | | | |
| dSpCas9-A1343-N | | | | | | | | | |
| nogRNA-G1322spilt-KKH- | A | 0.00% | 0.02% | 99.80% | 0.00% | 0.01% | 99.83% | 0.00% | 0.01% |
| Cas9-G1322-N and | | | | | | | | | |
| dSpCas9-G1322-C | | | | | | | | | |
| nogRNA-G1322spilt-KKH- | T | 0.00% | 0.00% | 0.01% | 0.01% | 0.01% | 0.01% | 0.00% | 0.01% |
| Cas9-G1322-N and | | | | | | | | | |
| dSpCas9-G1322-C | | | | | | | | | |
| nogRNA-G1322spilt-KKH- | C | 0.00% | 0.00% | 0.02% | 0.00% | 0.00% | 0.03% | 0.00% | 0.01% |
| Cas9-G1322-N and | | | | | | | | | |
| dSpCas9-G1322-C | | | | | | | | | |
| nogRNA-G1322spilt-KKH- | G | 99.99% | 99.98% | 0.17% | 99.99% | 99.98% | 0.13% | 100.00% | 99.98% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | A | 0.01% | 0.04% | 99.78% | 0.00% | 0.01% | 99.82% | 0.00% | 0.01% |
| Cas9-G1322-C and | | | | | | | | | |
| dSpCas9-G1322-N | | | | | | | | | |
| nogRNA-G1322spilt-KKH- | T | 0.01% | 0.00% | 0.02% | 0.00% | 0.00% | 0.01% | 0.00% | 0.02% |
| Cas9-G1322-C and | | | | | | | | | |
| dSpCas9-G1322-N | | | | | | | | | |
| nogRNA-G1322spilt-KKH- | C | 0.00% | 0.00% | 0.01% | 0.00% | 0.00% | 0.06% | 0.00% | 0.00% |
| Cas9-G1322-C and | | | | | | | | | |
| dSpCas9-G1322-N | | | | | | | | | |
| nogRNA-G1322spilt-KKH- | G | 99.98% | 99.96% | 0.19% | 100.00% | 99.99% | 0.10% | 99.99% | 99.97% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1333spilt-KKH- | A | 0.00% | 0.02% | 99.74% | 0.01% | 0.02% | 99.80% | 0.01% | 0.01% |
| Cas9-G1333-N and | | | | | | | | | |
| dSpCas9-G1333-C | | | | | | | | | |
| nogRNA-G1333spilt-KKH- | T | 0.01% | 0.00% | 0.00% | 0.02% | 0.01% | 0.02% | 0.00% | 0.00% |
| Cas9-G1333-N and | | | | | | | | | |
| dSpCas9-G1333-C | | | | | | | | | |
| nogRNA-G1333spilt-KKH- | C | 0.00% | 0.00% | 0.04% | 0.00% | 0.00% | 0.04% | 0.00% | 0.00% |
| Cas9-G1333-N and | | | | | | | | | |
| dSpCas9-G1333-C | | | | | | | | | |
| nogRNA-G1333spilt-KKH- | G | 99.99% | 99.98% | 0.21% | 99.96% | 99.97% | 0.14% | 99.99% | 99.99% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | A | 0.00% | 0.03% | 99.78% | 0.01% | 0.02% | 99.85% | 0.01% | 0.01% |
| Cas9-G1333-C and | | | | | | | | | |
| dSpCas9-G1333-N | | | | | | | | | |
| nogRNA-G1333spilt-KKH- | T | 0.01% | 0.00% | 0.01% | 0.01% | 0.01% | 0.01% | 0.00% | 0.00% |
| Cas9-G1333-C and | | | | | | | | | |
| dSpCas9-G1333-N | | | | | | | | | |
| nogRNA-G1333spilt-KKH- | C | 0.00% | 0.01% | 0.05% | 0.00% | 0.00% | 0.04% | 0.00% | 0.00% |
| Cas9-G1333-C and | | | | | | | | | |
| dSpCas9-G1333-N | | | | | | | | | |
| nogRNA-G1333spilt-KKH- | G | 99.98% | 99.95% | 0.15% | 99.97% | 99.97% | 0.10% | 99.99% | 99.99% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1371spilt-KKH- | A | 0.00% | 0.00% | 99.79% | 0.00% | 0.01% | 99.82% | 0.02% | 0.01% |
| Cas9-G1371-N and | | | | | | | | | |
| dSpCas9-G1371-C | | | | | | | | | |
| nogRNA-G1371spilt-KKH- | T | 0.01% | 0.02% | 0.01% | 0.00% | 0.01% | 0.02% | 0.01% | 0.01% |
| Cas9-G1371-N and | | | | | | | | | |
| dSpCas9-G1371-C | | | | | | | | | |
| nogRNA-G1371spilt-KKH- | C | 0.00% | 0.00% | 0.04% | 0.00% | 0.01% | 0.03% | 0.00% | 0.00% |
| Cas9-G1371-N and | | | | | | | | | |
| dSpCas9-G1371-C | | | | | | | | | |
| nogRNA-G1371spilt-KKH- | G | 99.99% | 99.98% | 0.16% | 100.00% | 99.97% | 0.13% | 99.97% | 99.97% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.00% | 0.00% | 0.00% | 0.00% | 0.00% | 0.00% | 0.01% | 0.01% |
| Cas9-G1371-N and | | | | | | | | | |
| dSpCas9-G1371-C | | | | | | | | | |
| nogRNA-G1371spilt-KKH- | A | 0.00% | 0.01% | 99.79% | 0.00% | 0.00% | 99.80% | 0.01% | 0.00% |
| Cas9-G1371-C and | | | | | | | | | |
| dSpCas9-G1371-N | | | | | | | | | |
| nogRNA-G1371spilt-KKH- | T | 0.02% | 0.01% | 0.01% | 0.01% | 0.02% | 0.00% | 0.02% | 0.01% |
| Cas9-G1371-C and | | | | | | | | | |
| dSpCas9-G1371-N | | | | | | | | | |
| nogRNA-G1371spilt-KKH- | C | 0.00% | 0.00% | 0.03% | 0.00% | 0.00% | 0.05% | 0.00% | 0.00% |
| Cas9-G1371-C and | | | | | | | | | |
| dSpCas9-G1371-N | | | | | | | | | |
| nogRNA-G1371spilt-KKH- | G | 99.97% | 99.98% | 0.17% | 99.99% | 99.98% | 0.15% | 99.95% | 99.98% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1371spilt-KKH- | — | 0.00% | 0.00% | 0.00% | 0.00% | 0.00% | 0.00% | 0.01% | 0.01% |
| Cas9-G1371-C and | | | | | | | | | |
| dSpCas9-G1371-N | | | | | | | | | |
| nogRNA-G1397spilt-KKH- | A | 0.01% | 0.00% | 99.79% | 0.01% | 0.02% | 99.86% | 0.01% | 0.01% |
| Cas9-G1397-N and | | | | | | | | | |
| dSpCas9-G1397-C | | | | | | | | | |
| nogRNA-G1397spilt-KKH- | T | 0.01% | 0.01% | 0.00% | 0.00% | 0.02% | 0.01% | 0.01% | 0.00% |
| Cas9-G1397-N and | | | | | | | | | |
| dSpCas9-G1397-C | | | | | | | | | |
| nogRNA-G1397spilt-KKH- | C | 0.00% | 0.00% | 0.04% | 0.00% | 0.00% | 0.02% | 0.00% | 0.00% |
| Cas9-G1397-N and | | | | | | | | | |
| dSpCas9-G1397-C | | | | | | | | | |
| nogRNA-G1397spilt-KKH- | G | 99.98% | 99.99% | 0.16% | 99.98% | 99.97% | 0.10% | 99.97% | 99.98% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.00% | 0.00% | 0.00% | 0.00% | 0.00% | 0.00% | 0.01% | 0.01% |
| Cas9-G1397-N and | | | | | | | | | |
| dSpCas9-G1397-C | | | | | | | | | |
| nogRNA-G1397spilt-KKH- | A | 0.01% | 0.02% | 99.78% | 0.01% | 0.01% | 99.84% | 0.01% | 0.00% |
| Cas9-G1397-C and | | | | | | | | | |
| dSpCas9-G1397-N | | | | | | | | | |
| nogRNA-G1397spilt-KKH- | T | 0.01% | 0.00% | 0.02% | 0.01% | 0.01% | 0.01% | 0.00% | 0.00% |
| Cas9-G1397-C and | | | | | | | | | |
| dSpCas9-G1397-N | | | | | | | | | |
| nogRNA-G1397spilt-KKH- | C | 0.00% | 0.00% | 0.05% | 0.00% | 0.00% | 0.06% | 0.00% | 0.00% |
| Cas9-G1397-C and | | | | | | | | | |
| dSpCas9-G1397-N | | | | | | | | | |
| nogRNA-G1397spilt-KKH- | G | 99.98% | 99.97% | 0.16% | 99.98% | 99.98% | 0.09% | 99.99% | 100.00% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.00% | 0.00% | 0.00% | 0.00% | 0.00% | 0.01% | 0.00% | 0.00% |
| Cas9-G1397-C and | | | | | | | | | |
| dSpCas9-G1397-N | | | | | | | | | |
| nogRNA-N1357spilt-KKH- | A | 0.02% | 0.01% | 99.81% | 0.01% | 0.01% | 99.85% | 0.00% | 0.00% |
| Cas9-N1357-N and | | | | | | | | | |
| dSpCas9-N1357-C | | | | | | | | | |
| nogRNA-N1357spilt-KKH- | T | 0.01% | 0.00% | 0.00% | 0.02% | 0.02% | 0.01% | 0.01% | 0.00% |
| Cas9-N1357-N and | | | | | | | | | |
| dSpCas9-N1357-C | | | | | | | | | |
| nogRNA-N1357spilt-KKH- | C | 0.00% | 0.00% | 0.02% | 0.00% | 0.00% | 0.03% | 0.00% | 0.00% |
| Cas9-N1357-N and | | | | | | | | | |
| dSpCas9-N1357-C | | | | | | | | | |
| nogRNA-N1357spilt-KKH- | G | 99.97% | 99.99% | 0.17% | 99.98% | 99.97% | 0.11% | 99.98% | 99.99% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | A | 0.00% | 0.01% | 99.71% | 0.00% | 0.00% | 99.76% | 0.00% | 0.01% |
| Cas9-N1357-C and | | | | | | | | | |
| dSpCas9-N1357-N | | | | | | | | | |
| nogRNA-N1357spilt-KKH- | T | 0.01% | 0.01% | 0.01% | 0.03% | 0.01% | 0.01% | 0.01% | 0.01% |
| Cas9-N1357-C and | | | | | | | | | |
| dSpCas9-N1357-N | | | | | | | | | |
| nogRNA-N1357spilt-KKH- | C | 0.01% | 0.00% | 0.06% | 0.01% | 0.00% | 0.08% | 0.00% | 0.01% |
| Cas9-N1357-C and | | | | | | | | | |
| dSpCas9-N1357-N | | | | | | | | | |
| nogRNA-N1357spilt-KKH- | G | 99.99% | 99.98% | 0.22% | 99.96% | 99.98% | 0.15% | 99.98% | 99.96% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.00% | 0.00% | 0.01% | 0.00% | 0.01% | 0.00% | 0.01% | 0.02% |
| Cas9-N1357-C and | | | | | | | | | |
| dSpCas9-N1357-N | | | | | | | | | |
| nogRNA-N1387spilt-KKH- | A | 0.01% | 0.01% | 99.79% | 0.00% | 0.00% | 99.81% | 0.00% | 0.01% |
| Cas9-N1387-N and | | | | | | | | | |
| dSpCas9-N1387-C | | | | | | | | | |
| nogRNA-N1387spilt-KKH- | T | 0.00% | 0.02% | 0.01% | 0.00% | 0.00% | 0.01% | 0.02% | 0.00% |
| Cas9-N1387-N and | | | | | | | | | |
| dSpCas9-N1387-C | | | | | | | | | |
| nogRNA-N1387spilt-KKH- | C | 0.00% | 0.00% | 0.04% | 0.00% | 0.00% | 0.04% | 0.00% | 0.00% |
| Cas9-N1387-N and | | | | | | | | | |
| dSpCas9-N1387-C | | | | | | | | | |
| nogRNA-N1387spilt-KKH- | G | 99.98% | 99.97% | 0.15% | 99.99% | 99.99% | 0.14% | 99.97% | 99.98% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | A | 0.00% | 0.00% | 99.74% | 0.01% | 0.01% | 99.82% | 0.00% | 0.00% |
| Cas9-N1387-C and | | | | | | | | | |
| dSpCas9-N1387-N | | | | | | | | | |
| nogRNA-N1387spilt-KKH- | T | 0.01% | 0.01% | 0.02% | 0.01% | 0.01% | 0.01% | 0.01% | 0.00% |
| Cas9-N1387-C and | | | | | | | | | |
| dSpCas9-N1387-N | | | | | | | | | |
| nogRNA-N1387spilt-KKH- | C | 0.00% | 0.00% | 0.04% | 0.00% | 0.00% | 0.04% | 0.00% | 0.00% |
| Cas9-N1387-C and | | | | | | | | | |
| dSpCas9-N1387-N | | | | | | | | | |
| nogRNA-N1387spilt-KKH- | G | 99.99% | 99.98% | 0.20% | 99.97% | 99.97% | 0.13% | 99.98% | 99.99% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |

| Batch | Nucleotide | A | A | G | G | G | C | C |
|---|---|---|---|---|---|---|---|---|
| nogRNA-A1343spilt-KKH- | A | 99.87% | 99.97% | 0.01% | 0.01% | 0.01% | 0.03% | 0.02% |
| Cas9-A1343-N and | | | | | | | | |
| dSpCas9-A1343-C | | | | | | | | |
| nogRNA-A1343spilt-KKH- | T | 0.02% | 0.00% | 0.01% | 0.01% | 0.00% | 0.05% | 0.05% |
| Cas9-A1343-N and | | | | | | | | |
| dSpCas9-A1343-C | | | | | | | | |
| nogRNA-A1343spilt-KKH- | C | 0.00% | 0.00% | 0.00% | 0.00% | 0.01% | 99.88% | 99.90% |
| Cas9-A1343-N and | | | | | | | | |
| dSpCas9-A1343-C | | | | | | | | |
| nogRNA-A1343spilt-KKH- | G | 0.11% | 0.02% | 99.98% | 99.98% | 99.98% | 0.03% | 0.02% |
| Cas9-A1343-N and | | | | | | | | |
| dSpCas9-A1343-C | | | | | | | | |
| nogRNA-A1343spilt-KKH- | N | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | — | 0.00% | 0.00% | 0.00% | 0.00% | 0.00% | 0.00% | 0.00% |
| Cas9-A1343-N and | | | | | | | | |
| dSpCas9-A1343-C | | | | | | | | |
| nogRNA-A1343spilt-KKH- | A | 99.80% | 99.89% | 0.03% | 0.00% | 0.01% | 0.03% | 0.02% |
| Cas9-A1343-C and | | | | | | | | |
| dSpCas9-A1343-N | | | | | | | | |
| nogRNA-A1343spilt-KKH- | T | 0.00% | 0.01% | 0.01% | 0.00% | 0.01% | 0.04% | 0.07% |
| Cas9-A1343-C and | | | | | | | | |
| dSpCas9-A1343-N | | | | | | | | |
| nogRNA-A1343spilt-KKH- | C | 0.05% | 0.06% | 0.00% | 0.00% | 0.00% | 99.91% | 99.90% |
| Cas9-A1343-C and | | | | | | | | |
| dSpCas9-A1343-N | | | | | | | | |
| nogRNA-A1343spilt-KKH- | G | 0.13% | 0.04% | 99.95% | 99.99% | 99.98% | 0.03% | 0.01% |
| Cas9-A1343-C and | | | | | | | | |
| dSpCas9-A1343-N | | | | | | | | |
| nogRNA-A1343spilt-KKH- | N | 0.00% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-A1343spilt-KKH- | — | 0.01% | 0.00% | 0.00% | 0.00% | 0.00% | 0.00% | 0.00% |
| Cas9-A1343-C and | | | | | | | | |
| dSpCas9-A1343-N | | | | | | | | |
| nogRNA-G1322spilt-KKH- | A | 99.88% | 99.95% | 0.03% | 0.00% | 0.00% | 0.01% | 0.03% |
| Cas9-G1322-N and | | | | | | | | |
| dSpCas9-G1322-C | | | | | | | | |
| nogRNA-G1322spilt-KKH- | T | 0.00% | 0.02% | 0.00% | 0.01% | 0.00% | 0.03% | 0.09% |
| Cas9-G1322-N and | | | | | | | | |
| dSpCas9-G1322-C | | | | | | | | |
| nogRNA-G1322spilt-KKH- | C | 0.02% | 0.01% | 0.00% | 0.00% | 0.00% | 99.93% | 99.86% |
| Cas9-G1322-N and | | | | | | | | |
| dSpCas9-G1322-C | | | | | | | | |
| nogRNA-G1322spilt-KKH- | G | 0.09% | 0.01% | 99.97% | 99.99% | 99.99% | 0.03% | 0.01% |
| Cas9-G1322-N and | | | | | | | | |
| dSpCas9-G1322-C | | | | | | | | |
| nogRNA-G1322spilt-KKH- | N | 0.00% | 0.00% | 0.00% | 0.00% | 0.00% | 0.00% | 0.00% |
| Cas9-G1322-N and | | | | | | | | |
| dSpCas9-G1322-C | | | | | | | | |
| nogRNA-G1322spilt-KKH- | — | 0.00% | 0.00% | 0.00% | 0.00% | 0.01% | 0.00% | 0.01% |
| Cas9-G1322-N and | | | | | | | | |
| dSpCas9-G1322-C | | | | | | | | |
| nogRNA-G1322spilt-KKH- | A | 99.77% | 99.92% | 0.02% | 0.00% | 0.01% | 0.03% | 0.02% |
| Cas9-G1322-C and | | | | | | | | |
| dSpCas9-G1322-N | | | | | | | | |
| nogRNA-G1322spilt-KKH- | T | 0.02% | 0.01% | 0.04% | 0.01% | 0.00% | 0.05% | 0.10% |
| Cas9-G1322-C and | | | | | | | | |
| dSpCas9-G1322-N | | | | | | | | |
| nogRNA-G1322spilt-KKH- | C | 0.03% | 0.01% | 0.00% | 0.00% | 0.00% | 99.88% | 99.86% |
| Cas9-G1322-C and | | | | | | | | |
| dSpCas9-G1322-N | | | | | | | | |
| nogRNA-G1322spilt-KKH- | G | 0.17% | 0.05% | 99.94% | 99.99% | 99.99% | 0.05% | 0.02% |
| Cas9-G1322-C and | | | | | | | | |
| dSpCas9-G1322-N | | | | | | | | |
| nogRNA-G1322spilt-KKH- | N | 0.00% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1322spilt-KKH- | — | 0.01% | 0.00% | 0.00% | 0.00% | 0.00% | 0.00% | 0.00% |
| Cas9-G1322-C and | | | | | | | | |
| dSpCas9-G1322-N | | | | | | | | |
| nogRNA-G1333spilt-KKH- | A | 99.80% | 99.91% | 0.03% | 0.01% | 0.00% | 0.02% | 0.03% |
| Cas9-G1333-N and | | | | | | | | |
| dSpCas9-G1333-C | | | | | | | | |
| nogRNA-G1333spilt-KKH- | T | 0.02% | 0.01% | 0.01% | 0.00% | 0.02% | 0.03% | 0.06% |
| Cas9-G1333-N and | | | | | | | | |
| dSpCas9-G1333-C | | | | | | | | |
| nogRNA-G1333spilt-KKH- | C | 0.00% | 0.02% | 0.00% | 0.00% | 0.00% | 99.90% | 99.88% |
| Cas9-G1333-N and | | | | | | | | |
| dSpCas9-G1333-C | | | | | | | | |
| nogRNA-G1333spilt-KKH- | G | 0.18% | 0.06% | 99.96% | 99.98% | 99.97% | 0.05% | 0.01% |
| Cas9-G1333-N and | | | | | | | | |
| dSpCas9-G1333-C | | | | | | | | |
| nogRNA-G1333spilt-KKH- | N | 0.00% | 0.00% | 0.00% | 0.00% | 0.00% | 0.00% | 0.00% |
| Cas9-G1333-N and | | | | | | | | |
| dSpCas9-G1333-C | | | | | | | | |
| nogRNA-G1333spilt-KKH- | — | 0.00% | 0.00% | 0.00% | 0.00% | 0.00% | 0.00% | 0.01% |
| Cas9-G1333-N and | | | | | | | | |
| dSpCas9-G1333-C | | | | | | | | |
| nogRNA-G1333spilt-KKH- | A | 99.83% | 99.96% | 0.00% | 0.02% | 0.00% | 0.01% | 0.02% |
| Cas9-G1333-C and | | | | | | | | |
| dSpCas9-G1333-N | | | | | | | | |
| nogRNA-G1333spilt-KKH- | T | 0.03% | 0.01% | 0.01% | 0.00% | 0.00% | 0.04% | 0.09% |
| Cas9-G1333-C and | | | | | | | | |
| dSpCas9-G1333-N | | | | | | | | |
| nogRNA-G1333spilt-KKH- | C | 0.01% | 0.01% | 0.01% | 0.00% | 0.00% | 99.92% | 99.88% |
| Cas9-G1333-C and | | | | | | | | |
| dSpCas9-G1333-N | | | | | | | | |
| nogRNA-G1333spilt-KKH- | G | 0.13% | 0.02% | 99.98% | 99.97% | 99.99% | 0.03% | 0.00% |
| Cas9-G1333-C and | | | | | | | | |
| dSpCas9-G1333-N | | | | | | | | |
| nogRNA-G1333spilt-KKH- | N | 0.00% | 0.00% | 0.00% | 0.00% | 0.00% | 0.00% | 0.00% |
| Cas9-G1333-C and | | | | | | | | |
| dSpCas9-G1333-N | | | | | | | | |
| nogRNA-G1333spilt-KKH- | — | 0.00% | 0.00% | 0.00% | 0.00% | 0.01% | 0.00% | 0.01% |
| Cas9-G1333-C and | | | | | | | | |
| dSpCas9-G1333-N | | | | | | | | |
| nogRNA-G1371spilt-KKH- | A | 99.82% | 99.94% | 0.02% | 0.02% | 0.00% | 0.02% | 0.01% |
| Cas9-G1371-N and | | | | | | | | |
| dSpCas9-G1371-C | | | | | | | | |
| nogRNA-G1371spilt-KKH- | T | 0.01% | 0.00% | 0.01% | 0.00% | 0.00% | 0.05% | 0.08% |
| Cas9-G1371-N and | | | | | | | | |
| dSpCas9-G1371-C | | | | | | | | |
| nogRNA-G1371spilt-KKH- | C | 0.02% | 0.01% | 0.00% | 0.00% | 0.00% | 99.89% | 99.87% |
| Cas9-G1371-N and | | | | | | | | |
| dSpCas9-G1371-C | | | | | | | | |
| nogRNA-G1371spilt-KKH- | G | 0.14% | 0.04% | 99.97% | 99.98% | 99.98% | 0.03% | 0.02% |
| Cas9-G1371-N and | | | | | | | | |
| dSpCas9-G1371-C | | | | | | | | |
| nogRNA-G1371spilt-KKH- | N | 0.00% | 0.00% | 0.00% | 0.00% | 0.00% | 0.00% | 0.00% |
| Cas9-G1371-N and | | | | | | | | |
| dSpCas9-G1371-C | | | | | | | | |
| nogRNA-G1371spilt-KKH- | — | 0.01% | 0.00% | 0.00% | 0.00% | 0.01% | 0.00% | 0.02% |
| Cas9-G1371-N and | | | | | | | | |
| dSpCas9-G1371-C | | | | | | | | |
| nogRNA-G1371spilt-KKH- | A | 99.83% | 99.92% | 0.02% | 0.02% | 0.00% | 0.02% | 0.01% |
| Cas9-G1371-C and | | | | | | | | |
| dSpCas9-G1371-N | | | | | | | | |
| nogRNA-G1371spilt-KKH- | T | 0.01% | 0.01% | 0.01% | 0.00% | 0.01% | 0.06% | 0.09% |
| Cas9-G1371-C and | | | | | | | | |
| dSpCas9-G1371-N | | | | | | | | |
| nogRNA-G1371spilt-KKH- | C | 0.01% | 0.02% | 0.00% | 0.01% | 0.00% | 99.88% | 99.89% |
| Cas9-G1371-C and | | | | | | | | |
| dSpCas9-G1371-N | | | | | | | | |
| nogRNA-G1371spilt-KKH- | G | 0.14% | 0.05% | 99.96% | 99.97% | 99.99% | 0.03% | 0.00% |
| Cas9-G1371-C and | | | | | | | | |
| dSpCas9-G1371-N | | | | | | | | |
| nogRNA-G1371spilt-KKH- | N | 0.00% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1371spilt-KKH- | — | 0.01% | 0.00% | 0.00% | 0.00% | 0.00% | 0.00% | 0.00% |
| Cas9-G1371-C and | | | | | | | | |
| dSpCas9-G1371-N | | | | | | | | |
| nogRNA-G1397spilt-KKH- | A | 99.83% | 99.93% | 0.02% | 0.02% | 0.01% | 0.05% | 0.02% |
| Cas9-G1397-N and | | | | | | | | |
| dSpCas9-G1397-C | | | | | | | | |
| nogRNA-G1397spilt-KKH- | T | 0.01% | 0.00% | 0.00% | 0.00% | 0.00% | 0.06% | 0.09% |
| Cas9-G1397-N and | | | | | | | | |
| dSpCas9-G1397-C | | | | | | | | |
| nogRNA-G1397spilt-KKH- | C | 0.02% | 0.01% | 0.00% | 0.00% | 0.01% | 99.87% | 99.87% |
| Cas9-G1397-N and | | | | | | | | |
| dSpCas9-G1397-C | | | | | | | | |
| nogRNA-G1397spilt-KKH- | G | 0.13% | 0.05% | 99.98% | 99.97% | 99.98% | 0.02% | 0.02% |
| Cas9-G1397-N and | | | | | | | | |
| dSpCas9-G1397-C | | | | | | | | |
| nogRNA-G1397spilt-KKH- | N | 0.00% | 0.00% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | — | 0.01% | 0.01% | 0.00% | 0.00% | 0.00% | 0.00% | 0.00% |
| Cas9-G1397-N and | | | | | | | | |
| dSpCas9-G1397-C | | | | | | | | |
| nogRNA-G1397spilt-KKH- | A | 99.78% | 99.95% | 0.01% | 0.01% | 0.01% | 0.02% | 0.03% |
| Cas9-G1397-C and | | | | | | | | |
| dSpCas9-G1397-N | | | | | | | | |
| nogRNA-G1397spilt-KKH- | T | 0.02% | 0.00% | 0.02% | 0.00% | 0.00% | 0.05% | 0.08% |
| Cas9-G1397-C and | | | | | | | | |
| dSpCas9-G1397-N | | | | | | | | |
| nogRNA-G1397spilt-KKH- | C | 0.02% | 0.01% | 0.00% | 0.00% | 0.00% | 99.89% | 99.86% |
| Cas9-G1397-C and | | | | | | | | |
| dSpCas9-G1397-N | | | | | | | | |
| nogRNA-G1397spilt-KKH- | G | 0.18% | 0.03% | 99.98% | 99.99% | 99.99% | 0.03% | 0.02% |
| Cas9-G1397-C and | | | | | | | | |
| dSpCas9-G1397-N | | | | | | | | |
| nogRNA-G1397spilt-KKH- | N | 0.00% | 0.00% | 0.00% | 0.00% | 0.00% | 0.00% | 0.00% |
| Cas9-G1397-C and | | | | | | | | |
| dSpCas9-G1397-N | | | | | | | | |
| nogRNA-G1397spilt-KKH- | — | 0.00% | 0.00% | 0.00% | 0.00% | 0.00% | 0.00% | 0.01% |
| Cas9-G1397-C and | | | | | | | | |
| dSpCas9-G1397-N | | | | | | | | |
| nogRNA-N1357spilt-KKH- | A | 99.82% | 99.94% | 0.02% | 0.01% | 0.01% | 0.02% | 0.03% |
| Cas9-N1357-N and | | | | | | | | |
| dSpCas9-N1357-C | | | | | | | | |
| nogRNA-N1357spilt-KKH- | T | 0.02% | 0.01% | 0.01% | 0.01% | 0.01% | 0.05% | 0.11% |
| Cas9-N1357-N and | | | | | | | | |
| dSpCas9-N1357-C | | | | | | | | |
| nogRNA-N1357spilt-KKH- | C | 0.02% | 0.01% | 0.00% | 0.00% | 0.00% | 99.90% | 99.84% |
| Cas9-N1357-N and | | | | | | | | |
| dSpCas9-N1357-C | | | | | | | | |
| nogRNA-N1357spilt-KKH- | G | 0.15% | 0.05% | 99.96% | 99.98% | 99.97% | 0.02% | 0.01% |
| Cas9-N1357-N and | | | | | | | | |
| dSpCas9-N1357-C | | | | | | | | |
| nogRNA-N1357spilt-KKH- | N | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | — | 0.00% | 0.00% | 0.00% | 0.00% | 0.00% | 0.00% | 0.00% |
| Cas9-N1357-N and | | | | | | | | |
| dSpCas9-N1357-C | | | | | | | | |
| nogRNA-N1357spilt-KKH- | A | 99.65% | 99.92% | 0.02% | 0.00% | 0.01% | 0.03% | 0.01% |
| Cas9-N1357-C and | | | | | | | | |
| dSpCas9-N1357-N | | | | | | | | |
| nogRNA-N1357spilt-KKH- | T | 0.01% | 0.00% | 0.01% | 0.00% | 0.01% | 0.03% | 0.13% |
| Cas9-N1357-C and | | | | | | | | |
| dSpCas9-N1357-N | | | | | | | | |
| nogRNA-N1357spilt-KKH- | C | 0.07% | 0.04% | 0.00% | 0.00% | 0.00% | 99.92% | 99.84% |
| Cas9-N1357-C and | | | | | | | | |
| dSpCas9-N1357-N | | | | | | | | |
| nogRNA-N1357spilt-KKH- | G | 0.26% | 0.04% | 99.98% | 100.00% | 99.98% | 0.03% | 0.02% |
| Cas9-N1357-C and | | | | | | | | |
| dSpCas9-N1357-N | | | | | | | | |
| nogRNA-N1357spilt-KKH- | N | 0.00% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1357spilt-KKH- | — | 0.01% | 0.00% | 0.00% | 0.00% | 0.00% | 0.00% | 0.00% |
| Cas9-N1357-C and | | | | | | | | |
| dSpCas9-N1357-N | | | | | | | | |
| nogRNA-N1387spilt-KKH- | A | 99.79% | 99.93% | 0.02% | 0.01% | 0.00% | 0.03% | 0.02% |
| Cas9-N1387-N and | | | | | | | | |
| dSpCas9-N1387-C | | | | | | | | |
| nogRNA-N1387spilt-KKH- | T | 0.01% | 0.00% | 0.01% | 0.00% | 0.00% | 0.03% | 0.16% |
| Cas9-N1387-N and | | | | | | | | |
| dSpCas9-N1387-C | | | | | | | | |
| nogRNA-N1387spilt-KKH- | C | 0.04% | 0.00% | 0.00% | 0.00% | 0.00% | 99.92% | 99.78% |
| Cas9-N1387-N and | | | | | | | | |
| dSpCas9-N1387-C | | | | | | | | |
| nogRNA-N1387spilt-KKH- | G | 0.17% | 0.06% | 99.97% | 99.99% | 100.00% | 0.03% | 0.02% |
| Cas9-N1387-N and | | | | | | | | |
| dSpCas9-N1387-C | | | | | | | | |
| nogRNA-N1387spilt-KKH- | N | 0.00% | 0.00% | 0.00% | 0.00% | 0.00% | 0.00% | 0.00% |
| Cas9-N1387-N and | | | | | | | | |
| dSpCas9-N1387-C | | | | | | | | |
| nogRNA-N1387spilt-KKH- | — | 0.00% | 0.00% | 0.00% | 0.00% | 0.00% | 0.00% | 0.01% |
| Cas9-N1387-N and | | | | | | | | |
| dSpCas9-N1387-C | | | | | | | | |
| nogRNA-N1387spilt-KKH- | A | 99.79% | 99.93% | 0.02% | 0.01% | 0.00% | 0.03% | 0.01% |
| Cas9-N1387-C and | | | | | | | | |
| dSpCas9-N1387-N | | | | | | | | |
| nogRNA-N1387spilt-KKH- | T | 0.02% | 0.00% | 0.02% | 0.00% | 0.00% | 0.05% | 0.05% |
| Cas9-N1387-C and | | | | | | | | |
| dSpCas9-N1387-N | | | | | | | | |
| nogRNA-N1387spilt-KKH- | C | 0.01% | 0.01% | 0.00% | 0.00% | 0.00% | 99.89% | 99.91% |
| Cas9-N1387-C and | | | | | | | | |
| dSpCas9-N1387-N | | | | | | | | |
| nogRNA-N1387spilt-KKH- | G | 0.17% | 0.04% | 99.96% | 99.99% | 99.99% | 0.04% | 0.01% |
| Cas9-N1387-C and | | | | | | | | |
| dSpCas9-N1387-N | | | | | | | | |
| nogRNA-N1387spilt-KKH- | N | 0.00% | 0.00% | 0.00% | 0.00% | 0.00% | 0.00% | 0.00% |
| Cas9-N1387-C and | | | | | | | | |
| dSpCas9-N1387-N | | | | | | | | |
| nogRNA-N1387spilt-KKH- | — | 0.01% | 0.01% | 0.00% | 0.00% | 0.00% | 0.00% | 0.01% |
| Cas9-N1387-C and | | | | | | | | |
| dSpCas9-N1387-N | | | | | | | | |

| Batch | Nucleotide | T | G | A | G | T | C | C | G |
|---|---|---|---|---|---|---|---|---|---|
| nogRNA-A1343spilt-KKH- | A | 0.02% | 0.03% | 99.90% | 0.01% | 0.02% | 0.02% | 0.02% | 0.01% |
| Cas9-A1343-N and | | | | | | | | | |
| dSpCas9-A1343-C | | | | | | | | | |
| nogRNA-A1343spilt-KKH- | T | 99.88% | 0.01% | 0.00% | 0.00% | 99.83% | 0.06% | 0.05% | 0.01% |
| Cas9-A1343-N and | | | | | | | | | |
| dSpCas9-A1343-C | | | | | | | | | |
| nogRNA-A1343spilt-KKH- | C | 0.05% | 0.00% | 0.02% | 0.00% | 0.05% | 99.92% | 99.81% | 0.00% |
| Cas9-A1343-N and | | | | | | | | | |
| dSpCas9-A1343-C | | | | | | | | | |
| nogRNA-A1343spilt-KKH- | G | 0.05% | 99.96% | 0.07% | 99.95% | 0.04% | 0.00% | 0.03% | 99.92% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.00% | 0.00% | 0.00% | 0.04% | 0.07% | 0.00% | 0.09% | 0.05% |
| Cas9-A1343-N and | | | | | | | | | |
| dSpCas9-A1343-C | | | | | | | | | |
| nogRNA-A1343spilt-KKH- | A | 0.01% | 0.01% | 99.79% | 0.00% | 0.00% | 0.02% | 0.02% | 0.02% |
| Cas9-A1343-C and | | | | | | | | | |
| dSpCas9-A1343-N | | | | | | | | | |
| nogRNA-A1343spilt-KKH- | T | 99.92% | 0.02% | 0.01% | 0.00% | 99.82% | 0.04% | 0.08% | 0.01% |
| Cas9-A1343-C and | | | | | | | | | |
| dSpCas9-A1343-N | | | | | | | | | |
| nogRNA-A1343spilt-KKH- | C | 0.03% | 0.00% | 0.07% | 0.00% | 0.05% | 99.93% | 99.75% | 0.00% |
| Cas9-A1343-C and | | | | | | | | | |
| dSpCas9-A1343-N | | | | | | | | | |
| nogRNA-A1343spilt-KKH- | G | 0.04% | 99.96% | 0.12% | 99.97% | 0.03% | 0.01% | 0.03% | 99.90% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.00% | 0.00% | 0.00% | 0.01% | 0.09% | 0.00% | 0.13% | 0.06% |
| Cas9-A1343-C and | | | | | | | | | |
| dSpCas9-A1343-N | | | | | | | | | |
| nogRNA-G1322spilt-KKH- | A | 0.00% | 0.01% | 99.87% | 0.01% | 0.02% | 0.01% | 0.02% | 0.02% |
| Cas9-G1322-N and | | | | | | | | | |
| dSpCas9-G1322-C | | | | | | | | | |
| nogRNA-G1322spilt-KKH- | T | 99.93% | 0.01% | 0.00% | 0.00% | 99.83% | 0.04% | 0.08% | 0.01% |
| Cas9-G1322-N and | | | | | | | | | |
| dSpCas9-G1322-C | | | | | | | | | |
| nogRNA-G1322spilt-KKH- | C | 0.03% | 0.00% | 0.03% | 0.00% | 0.07% | 99.95% | 99.79% | 0.00% |
| Cas9-G1322-N and | | | | | | | | | |
| dSpCas9-G1322-C | | | | | | | | | |
| nogRNA-G1322spilt-KKH- | G | 0.03% | 99.97% | 0.10% | 99.96% | 0.05% | 0.00% | 0.02% | 99.89% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.00% | 0.00% | 0.00% | 0.03% | 0.03% | 0.00% | 0.10% | 0.08% |
| Cas9-G1322-N and | | | | | | | | | |
| dSpCas9-G1322-C | | | | | | | | | |
| nogRNA-G1322spilt-KKH- | A | 0.01% | 0.03% | 99.85% | 0.00% | 0.03% | 0.01% | 0.01% | 0.01% |
| Cas9-G1322-C and | | | | | | | | | |
| dSpCas9-G1322-N | | | | | | | | | |
| nogRNA-G1322spilt-KKH- | T | 99.87% | 0.03% | 0.00% | 0.00% | 99.81% | 0.06% | 0.05% | 0.01% |
| Cas9-G1322-C and | | | | | | | | | |
| dSpCas9-G1322-N | | | | | | | | | |
| nogRNA-G1322spilt-KKH- | C | 0.07% | 0.00% | 0.03% | 0.00% | 0.05% | 99.92% | 99.83% | 0.00% |
| Cas9-G1322-C and | | | | | | | | | |
| dSpCas9-G1322-N | | | | | | | | | |
| nogRNA-G1322spilt-KKH- | G | 0.04% | 99.94% | 0.11% | 99.97% | 0.06% | 0.00% | 0.02% | 99.89% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.00% | 0.00% | 0.01% | 0.02% | 0.05% | 0.00% | 0.09% | 0.09% |
| Cas9-G1322-C and | | | | | | | | | |
| dSpCas9-G1322-N | | | | | | | | | |
| nogRNA-G1333spilt-KKH- | A | 0.01% | 0.01% | 99.87% | 0.01% | 0.03% | 0.01% | 0.00% | 0.01% |
| Cas9-G1333-N and | | | | | | | | | |
| dSpCas9-G1333-C | | | | | | | | | |
| nogRNA-G1333spilt-KKH- | T | 99.94% | 0.00% | 0.00% | 0.00% | 99.85% | 0.04% | 0.03% | 0.00% |
| Cas9-G1333-N and | | | | | | | | | |
| dSpCas9-G1333-C | | | | | | | | | |
| nogRNA-G1333spilt-KKH- | C | 0.02% | 0.00% | 0.03% | 0.00% | 0.04% | 99.94% | 99.87% | 0.00% |
| Cas9-G1333-N and | | | | | | | | | |
| dSpCas9-G1333-C | | | | | | | | | |
| nogRNA-G1333spilt-KKH- | G | 0.02% | 99.99% | 0.09% | 99.96% | 0.04% | 0.01% | 0.00% | 99.91% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.00% | 0.00% | 0.01% | 0.03% | 0.04% | 0.00% | 0.09% | 0.07% |
| Cas9-G1333-N and | | | | | | | | | |
| dSpCas9-G1333-C | | | | | | | | | |
| nogRNA-G1333spilt-KKH- | A | 0.00% | 0.01% | 99.87% | 0.00% | 0.00% | 0.00% | 0.00% | 0.02% |
| Cas9-G1333-C and | | | | | | | | | |
| dSpCas9-G1333-N | | | | | | | | | |
| nogRNA-G1333spilt-KKH- | T | 99.91% | 0.00% | 0.00% | 0.01% | 99.85% | 0.06% | 0.02% | 0.01% |
| Cas9-G1333-C and | | | | | | | | | |
| dSpCas9-G1333-N | | | | | | | | | |
| nogRNA-G1333spilt-KKH- | C | 0.05% | 0.00% | 0.04% | 0.00% | 0.07% | 99.92% | 99.86% | 0.00% |
| Cas9-G1333-C and | | | | | | | | | |
| dSpCas9-G1333-N | | | | | | | | | |
| nogRNA-G1333spilt-KKH- | G | 0.04% | 99.99% | 0.08% | 99.92% | 0.04% | 0.01% | 0.01% | 99.90% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.00% | 0.00% | 0.00% | 0.06% | 0.04% | 0.00% | 0.10% | 0.06% |
| Cas9-G1333-C and | | | | | | | | | |
| dSpCas9-G1333-N | | | | | | | | | |
| nogRNA-G1371spilt-KKH- | A | 0.00% | 0.03% | 99.86% | 0.02% | 0.02% | 0.01% | 0.02% | 0.03% |
| Cas9-G1371-N and | | | | | | | | | |
| dSpCas9-G1371-C | | | | | | | | | |
| nogRNA-G1371spilt-KKH- | T | 99.92% | 0.00% | 0.00% | 0.01% | 99.84% | 0.04% | 0.05% | 0.00% |
| Cas9-G1371-N and | | | | | | | | | |
| dSpCas9-G1371-C | | | | | | | | | |
| nogRNA-G1371spilt-KKH- | C | 0.04% | 0.00% | 0.03% | 0.00% | 0.06% | 99.94% | 99.83% | 0.00% |
| Cas9-G1371-N and | | | | | | | | | |
| dSpCas9-G1371-C | | | | | | | | | |
| nogRNA-G1371spilt-KKH- | G | 0.03% | 99.97% | 0.11% | 99.95% | 0.04% | 0.00% | 0.00% | 99.91% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.00% | 0.00% | 0.00% | 0.02% | 0.05% | 0.01% | 0.11% | 0.06% |
| Cas9-G1371-N and | | | | | | | | | |
| dSpCas9-G1371-C | | | | | | | | | |
| nogRNA-G1371spilt-KKH- | A | 0.01% | 0.02% | 99.85% | 0.00% | 0.01% | 0.02% | 0.01% | 0.02% |
| Cas9-G1371-C and | | | | | | | | | |
| dSpCas9-G1371-N | | | | | | | | | |
| nogRNA-G1371spilt-KKH- | T | 99.91% | 0.02% | 0.01% | 0.01% | 99.82% | 0.05% | 0.04% | 0.01% |
| Cas9-G1371-C and | | | | | | | | | |
| dSpCas9-G1371-N | | | | | | | | | |
| nogRNA-G1371spilt-KKH- | C | 0.04% | 0.00% | 0.04% | 0.00% | 0.06% | 99.92% | 99.85% | 0.00% |
| Cas9-G1371-C and | | | | | | | | | |
| dSpCas9-G1371-N | | | | | | | | | |
| nogRNA-G1371spilt-KKH- | G | 0.05% | 99.95% | 0.11% | 99.98% | 0.05% | 0.00% | 0.02% | 99.91% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1371spilt-KKH- | — | 0.00% | 0.00% | 0.00% | 0.01% | 0.06% | 0.00% | 0.08% | 0.07% |
| Cas9-G1371-C and | | | | | | | | | |
| dSpCas9-G1371-N | | | | | | | | | |
| nogRNA-G1397spilt-KKH- | A | 0.01% | 0.01% | 99.84% | 0.01% | 0.00% | 0.02% | 0.00% | 0.02% |
| Cas9-G1397-N and | | | | | | | | | |
| dSpCas9-G1397-C | | | | | | | | | |
| nogRNA-G1397spilt-KKH- | T | 99.91% | 0.02% | 0.01% | 0.00% | 99.87% | 0.05% | 0.07% | 0.00% |
| Cas9-G1397-N and | | | | | | | | | |
| dSpCas9-G1397-C | | | | | | | | | |
| nogRNA-G1397spilt-KKH- | C | 0.05% | 0.00% | 0.03% | 0.00% | 0.04% | 99.92% | 99.83% | 0.00% |
| Cas9-G1397-N and | | | | | | | | | |
| dSpCas9-G1397-C | | | | | | | | | |
| nogRNA-G1397spilt-KKH- | G | 0.02% | 99.96% | 0.12% | 99.96% | 0.03% | 0.01% | 0.02% | 99.90% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.00% | 0.00% | 0.00% | 0.03% | 0.05% | 0.00% | 0.08% | 0.09% |
| Cas9-G1397-N and | | | | | | | | | |
| dSpCas9-G1397-C | | | | | | | | | |
| nogRNA-G1397spilt-KKH- | A | 0.01% | 0.01% | 99.87% | 0.00% | 0.02% | 0.02% | 0.02% | 0.03% |
| Cas9-G1397-C and | | | | | | | | | |
| dSpCas9-G1397-N | | | | | | | | | |
| nogRNA-G1397spilt-KKH- | T | 99.90% | 0.01% | 0.00% | 0.00% | 99.85% | 0.03% | 0.06% | 0.00% |
| Cas9-G1397-C and | | | | | | | | | |
| dSpCas9-G1397-N | | | | | | | | | |
| nogRNA-G1397spilt-KKH- | C | 0.05% | 0.00% | 0.04% | 0.00% | 0.05% | 99.94% | 99.79% | 0.00% |
| Cas9-G1397-C and | | | | | | | | | |
| dSpCas9-G1397-N | | | | | | | | | |
| nogRNA-G1397spilt-KKH- | G | 0.02% | 99.98% | 0.08% | 99.97% | 0.04% | 0.01% | 0.03% | 99.88% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.00% | 0.00% | 0.00% | 0.03% | 0.04% | 0.00% | 0.10% | 0.09% |
| Cas9-G1397-C and | | | | | | | | | |
| dSpCas9-G1397-N | | | | | | | | | |
| nogRNA-N1357spilt-KKH- | A | 0.02% | 0.03% | 99.84% | 0.01% | 0.01% | 0.01% | 0.00% | 0.02% |
| Cas9-N1357-N and | | | | | | | | | |
| dSpCas9-N1357-C | | | | | | | | | |
| nogRNA-N1357spilt-KKH- | T | 99.91% | 0.01% | 0.01% | 0.00% | 99.87% | 0.05% | 0.06% | 0.00% |
| Cas9-N1357-N and | | | | | | | | | |
| dSpCas9-N1357-C | | | | | | | | | |
| nogRNA-N1357spilt-KKH- | C | 0.03% | 0.00% | 0.04% | 0.00% | 0.04% | 99.94% | 99.84% | 0.00% |
| Cas9-N1357-N and | | | | | | | | | |
| dSpCas9-N1357-C | | | | | | | | | |
| nogRNA-N1357spilt-KKH- | G | 0.03% | 99.96% | 0.11% | 99.96% | 0.04% | 0.01% | 0.02% | 99.89% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.00% | 0.00% | 0.01% | 0.03% | 0.03% | 0.00% | 0.08% | 0.09% |
| Cas9-N1357-N and | | | | | | | | | |
| dSpCas9-N1357-C | | | | | | | | | |
| nogRNA-N1357spilt-KKH- | A | 0.01% | 0.01% | 99.80% | 0.01% | 0.02% | 0.02% | 0.02% | 0.01% |
| Cas9-N1357-C and | | | | | | | | | |
| dSpCas9-N1357-N | | | | | | | | | |
| nogRNA-N1357spilt-KKH- | T | 99.91% | 0.01% | 0.01% | 0.01% | 99.87% | 0.02% | 0.06% | 0.02% |
| Cas9-N1357-C and | | | | | | | | | |
| dSpCas9-N1357-N | | | | | | | | | |
| nogRNA-N1357spilt-KKH- | C | 0.03% | 0.00% | 0.06% | 0.00% | 0.04% | 99.96% | 99.80% | 0.01% |
| Cas9-N1357-C and | | | | | | | | | |
| dSpCas9-N1357-N | | | | | | | | | |
| nogRNA-N1357spilt-KKH- | G | 0.04% | 99.99% | 0.13% | 99.97% | 0.03% | 0.01% | 0.02% | 99.86% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.00% | 0.00% | 0.00% | 0.02% | 0.05% | 0.01% | 0.11% | 0.10% |
| Cas9-N1357-C and | | | | | | | | | |
| dSpCas9-N1357-N | | | | | | | | | |
| nogRNA-N1387spilt-KKH- | A | 0.00% | 0.01% | 99.82% | 0.00% | 0.02% | 0.03% | 0.03% | 0.02% |
| Cas9-N1387-N and | | | | | | | | | |
| dSpCas9-N1387-C | | | | | | | | | |
| nogRNA-N1387spilt-KKH- | T | 99.88% | 0.00% | 0.01% | 0.00% | 99.82% | 0.04% | 0.07% | 0.00% |
| Cas9-N1387-N and | | | | | | | | | |
| dSpCas9-N1387-C | | | | | | | | | |
| nogRNA-N1387spilt-KKH- | C | 0.07% | 0.00% | 0.03% | 0.00% | 0.08% | 99.94% | 99.77% | 0.00% |
| Cas9-N1387-N and | | | | | | | | | |
| dSpCas9-N1387-C | | | | | | | | | |
| nogRNA-N1387spilt-KKH- | G | 0.04% | 99.98% | 0.12% | 99.96% | 0.04% | 0.00% | 0.04% | 99.89% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.01% | 0.00% | 0.01% | 0.03% | 0.04% | 0.00% | 0.09% | 0.09% |
| Cas9-N1387-N and | | | | | | | | | |
| dSpCas9-N1387-C | | | | | | | | | |
| nogRNA-N1387spilt-KKH- | A | 0.00% | 0.03% | 99.86% | 0.01% | 0.01% | 0.01% | 0.00% | 0.01% |
| Cas9-N1387-C and | | | | | | | | | |
| dSpCas9-N1387-N | | | | | | | | | |
| nogRNA-N1387spilt-KKH- | T | 99.92% | 0.01% | 0.00% | 0.01% | 99.80% | 0.05% | 0.08% | 0.00% |
| Cas9-N1387-C and | | | | | | | | | |
| dSpCas9-N1387-N | | | | | | | | | |
| nogRNA-N1387spilt-KKH- | C | 0.04% | 0.00% | 0.02% | 0.00% | 0.06% | 99.93% | 99.76% | 0.00% |
| Cas9-N1387-C and | | | | | | | | | |
| dSpCas9-N1387-N | | | | | | | | | |
| nogRNA-N1387spilt-KKH- | G | 0.03% | 99.96% | 0.12% | 99.96% | 0.08% | 0.00% | 0.02% | 99.85% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.00% | 0.00% | 0.00% | 0.02% | 0.05% | 0.00% | 0.14% | 0.13% |
| Cas9-N1387-C and | | | | | | | | | |
| dSpCas9-N1387-N | | | | | | | | | |

| Batch | Nucleotide | A | G | C | A | G | A | A | G |
|---|---|---|---|---|---|---|---|---|---|
| nogRNA-A1343spilt-KKH- | A | 99.85% | 0.00% | 0.02% | 2.31% | 0.10% | 2.16% | 0.01% | 0.00% |
| Cas9-A1343-N and | | | | | | | | | |
| dSpCas9-A1343-C | | | | | | | | | |
| nogRNA-A1343spilt-KKH- | T | 0.01% | 0.01% | 0.02% | 0.01% | 0.01% | 0.00% | 0.00% | 0.00% |
| Cas9-A1343-N and | | | | | | | | | |
| dSpCas9-A1343-C | | | | | | | | | |
| nogRNA-A1343spilt-KKH- | C | 0.03% | 0.00% | 99.95% | 0.05% | 0.01% | 0.00% | 0.00% | 0.00% |
| Cas9-A1343-N and | | | | | | | | | |
| dSpCas9-A1343-C | | | | | | | | | |
| nogRNA-A1343spilt-KKH- | G | 0.07% | 99.96% | 0.00% | 0.10% | 2.34% | 0.06% | 0.00% | 0.01% |
| Cas9-A1343-N and | | | | | | | | | |
| dSpCas9-A1343-C | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-N and | | | | | | | | | |
| dSpCas9-A1343-C | | | | | | | | | |
| nogRNA-A1343spilt-KKH- | — | 0.03% | 0.03% | 0.00% | 97.53% | 97.54% | 97.77% | 99.99% | 99.99% |
| Cas9-A1343-N and | | | | | | | | | |
| dSpCas9-A1343-C | | | | | | | | | |
| nogRNA-A1343spilt-KKH- | A | 99.78% | 0.01% | 0.02% | 2.52% | 0.09% | 2.38% | 0.02% | 0.00% |
| Cas9-A1343-C and | | | | | | | | | |
| dSpCas9-A1343-N | | | | | | | | | |
| nogRNA-A1343spilt-KKH- | T | 0.00% | 0.01% | 0.04% | 0.01% | 0.01% | 0.00% | 0.00% | 0.00% |
| Cas9-A1343-C and | | | | | | | | | |
| dSpCas9-A1343-N | | | | | | | | | |
| nogRNA-A1343spilt-KKH- | C | 0.07% | 0.00% | 99.88% | 0.08% | 0.01% | 0.06% | 0.00% | 0.00% |
| Cas9-A1343-C and | | | | | | | | | |
| dSpCas9-A1343-N | | | | | | | | | |
| nogRNA-A1343spilt-KKH- | G | 0.11% | 99.93% | 0.02% | 0.09% | 2.57% | 0.05% | 0.00% | 0.02% |
| Cas9-A1343-C and | | | | | | | | | |
| dSpCas9-A1343-N | | | | | | | | | |
| nogRNA-A1343spilt-KKH- | N | 0.00% | 0.00% | 0.00% | 0.00% | 0.00% | 0.00% | 0.00% | 0.00% |
| Cas9-A1343-C and | | | | | | | | | |
| dSpCas9-A1343-N | | | | | | | | | |
| nogRNA-A1343spilt-KKH- | — | 0.04% | 0.05% | 0.04% | 97.29% | 97.33% | 97.51% | 99.98% | 99.98% |
| Cas9-A1343-C and | | | | | | | | | |
| dSpCas9-A1343-N | | | | | | | | | |
| nogRNA-G1322spilt-KKH- | A | 99.86% | 0.01% | 0.03% | 2.26% | 0.09% | 2.15% | 0.03% | 0.00% |
| Cas9-G1322-N and | | | | | | | | | |
| dSpCas9-G1322-C | | | | | | | | | |
| nogRNA-G1322spilt-KKH- | T | 0.01% | 0.00% | 0.04% | 0.02% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | C | 0.02% | 0.00% | 99.88% | 0.04% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | G | 0.09% | 99.94% | 0.03% | 0.12% | 2.32% | 0.03% | 0.00% | 0.03% |
| Cas9-G1322-N and | | | | | | | | | |
| dSpCas9-G1322-C | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-N and | | | | | | | | | |
| dSpCas9-G1322-C | | | | | | | | | |
| nogRNA-G1322spilt-KKH- | — | 0.02% | 0.04% | 0.02% | 97.56% | 97.59% | 97.82% | 99.97% | 99.97% |
| Cas9-G1322-N and | | | | | | | | | |
| dSpCas9-G1322-C | | | | | | | | | |
| nogRNA-G1322spilt-KKH- | A | 99.84% | 0.02% | 0.03% | 2.49% | 0.09% | 2.32% | 0.03% | 0.00% |
| Cas9-G1322-C and | | | | | | | | | |
| dSpCas9-G1322-N | | | | | | | | | |
| nogRNA-G1322spilt-KKH- | T | 0.01% | 0.01% | 0.03% | 0.02% | 0.02% | 0.00% | 0.00% | 0.00% |
| Cas9-G1322-C and | | | | | | | | | |
| dSpCas9-G1322-N | | | | | | | | | |
| nogRNA-G1322spilt-KKH- | C | 0.04% | 0.00% | 99.90% | 0.06% | 0.00% | 0.02% | 0.00% | 0.00% |
| Cas9-G1322-C and | | | | | | | | | |
| dSpCas9-G1322-N | | | | | | | | | |
| nogRNA-G1322spilt-KKH- | G | 0.08% | 99.93% | 0.00% | 0.10% | 2.54% | 0.09% | 0.00% | 0.02% |
| Cas9-G1322-C and | | | | | | | | | |
| dSpCas9-G1322-N | | | | | | | | | |
| nogRNA-G1322spilt-KKH- | N | 0.00% | 0.00% | 0.00% | 0.00% | 0.00% | 0.00% | 0.00% | 0.00% |
| Cas9-G1322-C and | | | | | | | | | |
| dSpCas9-G1322-N | | | | | | | | | |
| nogRNA-G1322spilt-KKH- | — | 0.03% | 0.04% | 0.05% | 97.33% | 97.35% | 97.58% | 99.97% | 99.98% |
| Cas9-G1322-C and | | | | | | | | | |
| dSpCas9-G1322-N | | | | | | | | | |
| nogRNA-G1333spilt-KKH- | A | 99.84% | 0.02% | 0.05% | 2.70% | 0.12% | 2.52% | 0.02% | 0.00% |
| Cas9-G1333-N and | | | | | | | | | |
| dSpCas9-G1333-C | | | | | | | | | |
| nogRNA-G1333spilt-KKH- | T | 0.01% | 0.00% | 0.03% | 0.01% | 0.01% | 0.00% | 0.00% | 0.00% |
| Cas9-G1333-N and | | | | | | | | | |
| dSpCas9-G1333-C | | | | | | | | | |
| nogRNA-G1333spilt-KKH- | C | 0.06% | 0.00% | 99.88% | 0.02% | 0.00% | 0.01% | 0.00% | 0.00% |
| Cas9-G1333-N and | | | | | | | | | |
| dSpCas9-G1333-C | | | | | | | | | |
| nogRNA-G1333spilt-KKH- | G | 0.07% | 99.92% | 0.02% | 0.09% | 2.67% | 0.06% | 0.00% | 0.02% |
| Cas9-G1333-N and | | | | | | | | | |
| dSpCas9-G1333-C | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-N and | | | | | | | | | |
| dSpCas9-G1333-C | | | | | | | | | |
| nogRNA-G1333spilt-KKH- | — | 0.02% | 0.06% | 0.02% | 97.19% | 97.21% | 97.40% | 99.98% | 99.98% |
| Cas9-G1333-N and | | | | | | | | | |
| dSpCas9-G1333-C | | | | | | | | | |
| nogRNA-G1333spilt-KKH- | A | 99.84% | 0.02% | 0.01% | 2.75% | 0.10% | 2.61% | 0.06% | 0.00% |
| Cas9-G1333-C and | | | | | | | | | |
| dSpCas9-G1333-N | | | | | | | | | |
| nogRNA-G1333spilt-KKH- | T | 0.02% | 0.00% | 0.04% | 0.00% | 0.01% | 0.00% | 0.00% | 0.00% |
| Cas9-G1333-C and | | | | | | | | | |
| dSpCas9-G1333-N | | | | | | | | | |
| nogRNA-G1333spilt-KKH- | C | 0.05% | 0.00% | 99.89% | 0.03% | 0.00% | 0.01% | 0.00% | 0.00% |
| Cas9-G1333-C and | | | | | | | | | |
| dSpCas9-G1333-N | | | | | | | | | |
| nogRNA-G1333spilt-KKH- | G | 0.08% | 99.95% | 0.01% | 0.10% | 2.77% | 0.05% | 0.00% | 0.05% |
| Cas9-G1333-C and | | | | | | | | | |
| dSpCas9-G1333-N | | | | | | | | | |
| nogRNA-G1333spilt-KKH- | N | 0.00% | 0.00% | 0.00% | 0.00% | 0.00% | 0.00% | 0.00% | 0.00% |
| Cas9-G1333-C and | | | | | | | | | |
| dSpCas9-G1333-N | | | | | | | | | |
| nogRNA-G1333spilt-KKH- | — | 0.02% | 0.03% | 0.04% | 97.12% | 97.13% | 97.33% | 99.94% | 99.95% |
| Cas9-G1333-C and | | | | | | | | | |
| dSpCas9-G1333-N | | | | | | | | | |
| nogRNA-G1371spilt-KKH- | A | 99.84% | 0.00% | 0.03% | 2.35% | 0.10% | 2.22% | 0.06% | 0.00% |
| Cas9-G1371-N and | | | | | | | | | |
| dSpCas9-G1371-C | | | | | | | | | |
| nogRNA-G1371spilt-KKH- | T | 0.02% | 0.00% | 0.03% | 0.01% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | C | 0.02% | 0.00% | 99.87% | 0.05% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | G | 0.07% | 99.95% | 0.03% | 0.11% | 2.38% | 0.06% | 0.00% | 0.06% |
| Cas9-G1371-N and | | | | | | | | | |
| dSpCas9-G1371-C | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-N and | | | | | | | | | |
| dSpCas9-G1371-C | | | | | | | | | |
| nogRNA-G1371spilt-KKH- | — | 0.04% | 0.05% | 0.04% | 97.48% | 97.51% | 97.72% | 99.94% | 99.94% |
| Cas9-G1371-N and | | | | | | | | | |
| dSpCas9-G1371-C | | | | | | | | | |
| nogRNA-G1371spilt-KKH- | A | 99.85% | 0.01% | 0.04% | 2.43% | 0.08% | 2.27% | 0.05% | 0.00% |
| Cas9-G1371-C and | | | | | | | | | |
| dSpCas9-G1371-N | | | | | | | | | |
| nogRNA-G1371spilt-KKH- | T | 0.01% | 0.01% | 0.07% | 0.01% | 0.01% | 0.00% | 0.00% | 0.00% |
| Cas9-G1371-C and | | | | | | | | | |
| dSpCas9-G1371-N | | | | | | | | | |
| nogRNA-G1371spilt-KKH- | C | 0.01% | 0.00% | 99.84% | 0.04% | 0.00% | 0.01% | 0.00% | 0.00% |
| Cas9-G1371-C and | | | | | | | | | |
| dSpCas9-G1371-N | | | | | | | | | |
| nogRNA-G1371spilt-KKH- | G | 0.09% | 99.93% | 0.03% | 0.10% | 2.45% | 0.05% | 0.00% | 0.06% |
| Cas9-G1371-C and | | | | | | | | | |
| dSpCas9-G1371-N | | | | | | | | | |
| nogRNA-G1371spilt-KKH- | N | 0.00% | 0.00% | 0.00% | 0.00% | 0.00% | 0.00% | 0.00% | 0.00% |
| Cas9-G1371-C and | | | | | | | | | |
| dSpCas9-G1371-N | | | | | | | | | |
| nogRNA-G1371spilt-KKH- | — | 0.03% | 0.05% | 0.02% | 97.43% | 97.46% | 97.66% | 99.94% | 99.94% |
| Cas9-G1371-C and | | | | | | | | | |
| dSpCas9-G1371-N | | | | | | | | | |
| nogRNA-G1397spilt-KKH- | A | 99.87% | 0.01% | 0.02% | 2.85% | 0.08% | 2.71% | 0.36% | 0.00% |
| Cas9-G1397-N and | | | | | | | | | |
| dSpCas9-G1397-C | | | | | | | | | |
| nogRNA-G1397spilt-KKH- | T | 0.00% | 0.00% | 0.04% | 0.02% | 0.01% | 0.00% | 0.00% | 0.00% |
| Cas9-G1397-N and | | | | | | | | | |
| dSpCas9-G1397-C | | | | | | | | | |
| nogRNA-G1397spilt-KKH- | C | 0.02% | 0.00% | 99.91% | 0.03% | 0.00% | 0.02% | 0.00% | 0.00% |
| Cas9-G1397-N and | | | | | | | | | |
| dSpCas9-G1397-C | | | | | | | | | |
| nogRNA-G1397spilt-KKH- | G | 0.09% | 99.94% | 0.02% | 0.09% | 2.87% | 0.05% | 0.00% | 0.36% |
| Cas9-G1397-N and | | | | | | | | | |
| dSpCas9-G1397-C | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-N and | | | | | | | | | |
| dSpCas9-G1397-C | | | | | | | | | |
| nogRNA-G1397spilt-KKH- | — | 0.01% | 0.05% | 0.01% | 97.01% | 97.04% | 97.23% | 99.64% | 99.64% |
| Cas9-G1397-N and | | | | | | | | | |
| dSpCas9-G1397-C | | | | | | | | | |
| nogRNA-G1397spilt-KKH- | A | 99.82% | 0.01% | 0.02% | 2.72% | 0.10% | 2.53% | 0.05% | 0.00% |
| Cas9-G1397-C and | | | | | | | | | |
| dSpCas9-G1397-N | | | | | | | | | |
| nogRNA-G1397spilt-KKH- | T | 0.01% | 0.00% | 0.02% | 0.01% | 0.01% | 0.01% | 0.00% | 0.00% |
| Cas9-G1397-C and | | | | | | | | | |
| dSpCas9-G1397-N | | | | | | | | | |
| nogRNA-G1397spilt-KKH- | C | 0.05% | 0.00% | 99.90% | 0.04% | 0.00% | 0.02% | 0.00% | 0.00% |
| Cas9-G1397-C and | | | | | | | | | |
| dSpCas9-G1397-N | | | | | | | | | |
| nogRNA-G1397spilt-KKH- | G | 0.07% | 99.93% | 0.02% | 0.09% | 2.72% | 0.08% | 0.00% | 0.05% |
| Cas9-G1397-C and | | | | | | | | | |
| dSpCas9-G1397-N | | | | | | | | | |
| nogRNA-G1397spilt-KKH- | N | 0.00% | 0.00% | 0.00% | 0.00% | 0.00% | 0.00% | 0.00% | 0.00% |
| Cas9-G1397-C and | | | | | | | | | |
| dSpCas9-G1397-N | | | | | | | | | |
| nogRNA-G1397spilt-KKH- | — | 0.05% | 0.06% | 0.04% | 97.14% | 97.17% | 97.35% | 99.95% | 99.95% |
| Cas9-G1397-C and | | | | | | | | | |
| dSpCas9-G1397-N | | | | | | | | | |
| nogRNA-N1357spilt-KKH- | A | 99.86% | 0.00% | 0.03% | 2.50% | 0.10% | 2.35% | 0.02% | 0.00% |
| Cas9-N1357-N and | | | | | | | | | |
| dSpCas9-N1357-C | | | | | | | | | |
| nogRNA-N1357spilt-KKH- | T | 0.01% | 0.01% | 0.05% | 0.01% | 0.00% | 0.01% | 0.00% | 0.00% |
| Cas9-N1357-N and | | | | | | | | | |
| dSpCas9-N1357-C | | | | | | | | | |
| nogRNA-N1357spilt-KKH- | C | 0.03% | 0.00% | 99.87% | 0.05% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | G | 0.09% | 99.95% | 0.01% | 0.13% | 2.57% | 0.05% | 0.00% | 0.02% |
| Cas9-N1357-N and | | | | | | | | | |
| dSpCas9-N1357-C | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-N and | | | | | | | | | |
| dSpCas9-N1357-C | | | | | | | | | |
| nogRNA-N1357spilt-KKH- | — | 0.02% | 0.03% | 0.03% | 97.31% | 97.33% | 97.59% | 99.98% | 99.98% |
| Cas9-N1357-N and | | | | | | | | | |
| dSpCas9-N1357-C | | | | | | | | | |
| nogRNA-N1357spilt-KKH- | A | 99.78% | 0.03% | 0.06% | 2.41% | 0.06% | 2.29% | 0.02% | 0.00% |
| Cas9-N1357-C and | | | | | | | | | |
| dSpCas9-N1357-N | | | | | | | | | |
| nogRNA-N1357spilt-KKH- | T | 0.01% | 0.01% | 0.03% | 0.02% | 0.01% | 0.01% | 0.00% | 0.00% |
| Cas9-N1357-C and | | | | | | | | | |
| dSpCas9-N1357-N | | | | | | | | | |
| nogRNA-N1357spilt-KKH- | C | 0.07% | 0.01% | 99.86% | 0.06% | 0.00% | 0.05% | 0.00% | 0.00% |
| Cas9-N1357-C and | | | | | | | | | |
| dSpCas9-N1357-N | | | | | | | | | |
| nogRNA-N1357spilt-KKH- | G | 0.08% | 99.90% | 0.03% | 0.05% | 2.45% | 0.04% | 0.00% | 0.02% |
| Cas9-N1357-C and | | | | | | | | | |
| dSpCas9-N1357-N | | | | | | | | | |
| nogRNA-N1357spilt-KKH- | N | 0.00% | 0.00% | 0.00% | 0.00% | 0.00% | 0.00% | 0.00% | 0.00% |
| Cas9-N1357-C and | | | | | | | | | |
| dSpCas9-N1357-N | | | | | | | | | |
| nogRNA-N1357spilt-KKH- | — | 0.06% | 0.06% | 0.02% | 97.46% | 97.48% | 97.62% | 99.98% | 99.98% |
| Cas9-N1357-C and | | | | | | | | | |
| dSpCas9-N1357-N | | | | | | | | | |
| nogRNA-N1387spilt-KKH- | A | 99.82% | 0.01% | 0.04% | 2.60% | 0.08% | 2.46% | 0.06% | 0.00% |
| Cas9-N1387-N and | | | | | | | | | |
| dSpCas9-N1387-C | | | | | | | | | |
| nogRNA-N1387spilt-KKH- | T | 0.01% | 0.03% | 0.03% | 0.00% | 0.01% | 0.00% | 0.00% | 0.00% |
| Cas9-N1387-N and | | | | | | | | | |
| dSpCas9-N1387-C | | | | | | | | | |
| nogRNA-N1387spilt-KKH- | C | 0.06% | 0.00% | 99.91% | 0.03% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | G | 0.08% | 99.92% | 0.01% | 0.12% | 2.64% | 0.05% | 0.00% | 0.06% |
| Cas9-N1387-N and | | | | | | | | | |
| dSpCas9-N1387-C | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-N and | | | | | | | | | |
| dSpCas9-N1387-C | | | | | | | | | |
| nogRNA-N1387spilt-KKH- | — | 0.03% | 0.04% | 0.01% | 97.24% | 97.27% | 97.48% | 99.94% | 99.94% |
| Cas9-N1387-N and | | | | | | | | | |
| dSpCas9-N1387-C | | | | | | | | | |
| nogRNA-N1387spilt-KKH- | A | 99.82% | 0.02% | 0.02% | 2.58% | 0.13% | 2.37% | 0.05% | 0.00% |
| Cas9-N1387-C and | | | | | | | | | |
| dSpCas9-N1387-N | | | | | | | | | |
| nogRNA-N1387spilt-KKH- | T | 0.01% | 0.00% | 0.04% | 0.00% | 0.01% | 0.00% | 0.00% | 0.00% |
| Cas9-N1387-C and | | | | | | | | | |
| dSpCas9-N1387-N | | | | | | | | | |
| nogRNA-N1387spilt-KKH- | C | 0.04% | 0.00% | 99.85% | 0.03% | 0.00% | 0.01% | 0.00% | 0.00% |
| Cas9-N1387-C and | | | | | | | | | |
| dSpCas9-N1387-N | | | | | | | | | |
| nogRNA-N1387spilt-KKH- | G | 0.11% | 99.93% | 0.02% | 0.13% | 2.57% | 0.08% | 0.00% | 0.05% |
| Cas9-N1387-C and | | | | | | | | | |
| dSpCas9-N1387-N | | | | | | | | | |
| nogRNA-N1387spilt-KKH- | N | 0.00% | 0.00% | 0.00% | 0.00% | 0.00% | 0.00% | 0.00% | 0.00% |
| Cas9-N1387-C and | | | | | | | | | |
| dSpCas9-N1387-N | | | | | | | | | |
| nogRNA-N1387spilt-KKH- | — | 0.02% | 0.04% | 0.06% | 97.27% | 97.29% | 97.53% | 99.94% | 99.95% |
| Cas9-N1387-C and | | | | | | | | | |
| dSpCas9-N1387-N | | | | | | | | | |

| Sample | Reads_aligned | Insertions | Deletions | indel % |
|---|---|---|---|---|
| ND5.1-DdCBE_replicate1 | 27776 | 0 | 4 | 0.014400922 |
| ND5.1-DdCBE_replicate2 | 35682 | 0 | 13 | 0.036432935 |
| ND5.1-DdCBE_replicate3 | 38221 | 0 | 7 | 0.018314539 |
| ND5.2-DdCBE_replicate1 | 75163 | 0 | 33 | 0.043904581 |
| ND5.2-DdCBE_replicate2 | 3150 | 0 | 1 | 0.031746032 |
| ND5.2-DdCBE_replicate3 | 100007 | 0 | 37 | 0.03699741 |
| ND5.3-DdCBE_replicate1 | 55241 | 0 | 10 | 0.018102496 |
| ND5.3-DdCBE_replicate2 | 64264 | 0 | 19 | 0.029565542 |
| ND5.3-DdCBE_replicate3 | 75963 | 0 | 18 | 0.023695747 |
| ND4-DdCBE_replicate1 | 96693 | 0 | 15 | 0.015513015 |
| ND4-DdCBE_replicate2 | 34406 | 0 | 9 | 0.026158228 |
| ND4-DdCBE_replicate3 | 67979 | 1 | 6 | 0.010297298 |
| ND2-DdCBE_replicate1 | 38026 | 0 | 8 | 0.021038237 |
| ND2-DdCBE_replicate2 | 42224 | 0 | 5 | 0.011841607 |
| ND2-DdCBE_replicate3 | 38272 | 0 | 7 | 0.018290134 |
| ND1-DdCBE_replicate1 | 3412 | 0 | 1 | 0.029308324 |
| ND1-DdCBE_replicate2 | 71984 | 0 | 13 | 0.018059569 |
| ND1-DdCBE_replicate3 | 27370 | 0 | 4 | 0.014614541 |
| BMATP8mito55mito56replicate1 | 46836 | 0 | 5 | 0.010675549 |
| BMATP8mito55mito56replicate2 | 47180 | 0 | 12 | 0.025434506 |
| BMATP8mito55mito56replicate3 | 50303 | 0 | 27 | 0.053674731 |
| untreated-day3_replicate1 | 50677 | 0 | 17 | 0.03354579 |
| untreated-day3_replicate2 | 81585 | 0 | 35 | 0.042900043 |
| untreated-day3_replicate3 | 70197 | 0 | 20 | 0.028491246 |
| untreated-day6_replicate1 | 96647 | 0 | 6 | 0.00620816 |
| untreated-day6_replicate2 | 35744 | 0 | 4 | 0.011190689 |
| untreated-day6_replicate3 | 57272 | 0 | 5 | 0.00873027 |

TABLE 12A
Base Percentages at TALE Binding Sites-ND6-DdCBE

| Batch | Nucleotide | T | C | A | C | C | A | A | G | A | C | C | T |
|---|---|---|---|---|---|---|---|---|---|---|---|---|---|
| ND6- | A | 0.01% | 0.01% | 99.94% | 0.01% | 0.01% | 99.94% | 99.93% | 0.09% | 99.95% | 0.02% | 0.01% | 0.01% |
| DdCBE_ | | | | | | | | | | | | | |
| replicate1 | | | | | | | | | | | | | |
| ND6- | T | 99.92% | 0.23% | 0.01% | 0.03% | 0.02% | 0.01% | 0.00% | 0.02% | 0.00% | 0.03% | 0.01% | 99.96% |
| DdCBE_ | | | | | | | | | | | | | |
| replicate1 | | | | | | | | | | | | | |
| ND6- | C | 0.07% | 99.76% | 0.04% | 99.94% | 99.96% | 0.03% | 0.04% | 0.00% | 0.03% | 99.95% | 99.99% | 0.02% |
| DdCBE_ | | | | | | | | | | | | | |
| replicate1 | | | | | | | | | | | | | |
| ND6- | G | 0.00% | 0.00% | 0.02% | 0.01% | 0.01% | 0.02% | 0.03% | 99.89% | 0.02% | 0.00% | 0.00% | 0.01% |
| DdCBE_ | | | | | | | | | | | | | |
| replicate1 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | A | 0.01% | 0.01% | 99.95% | 0.01% | 0.00% | 99.96% | 99.94% | 0.15% | 99.95% | 0.00% | 0.01% | 0.00% |
| DdCBE_ | | | | | | | | | | | | | |
| replicate2 | | | | | | | | | | | | | |
| ND6- | T | 99.90% | 0.21% | 0.01% | 0.02% | 0.02% | 0.01% | 0.00% | 0.02% | 0.02% | 0.03% | 0.00% | 99.99% |
| DdCBE_ | | | | | | | | | | | | | |
| replicate2 | | | | | | | | | | | | | |
| ND6- | C | 0.09% | 99.78% | 0.03% | 99.97% | 99.97% | 0.03% | 0.05% | 0.00% | 0.02% | 99.97% | 99.99% | 0.01% |
| DdCBE_ | | | | | | | | | | | | | |
| replicate2 | | | | | | | | | | | | | |
| ND6- | G | 0.00% | 0.00% | 0.01% | 0.00% | 0.00% | 0.01% | 0.01% | 99.83% | 0.01% | 0.00% | 0.00% | 0.00% |
| DdCBE_ | | | | | | | | | | | | | |
| replicate2 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | A | 0.01% | 0.00% | 99.9% | 0.01% | 0.02% | 99.95% | 99.95% | 0.13% | 99.98% | 0.01% | 0.00% | 0.01% |
| DdCBE_ | | | | | | | | | | | | | |
| replicate3 | | | | | | | | | | | | | |
| ND6- | T | 99.87% | 0.17% | 0.01% | 0.02% | 0.01% | 0.01% | 0.00% | 0.02% | 0.00% | 0.02% | 0.01% | 99.98% |
| DdCBE_ | | | | | | | | | | | | | |
| replicate3 | | | | | | | | | | | | | |
| ND6- | C | 0.11% | 99.82% | 0.03% | 99.97% | 99.96% | 0.03% | 0.04% | 0.00% | 0.01% | 99.97% | 99.99% | 0.01% |
| DdCBE_ | | | | | | | | | | | | | |
| replicate3 | | | | | | | | | | | | | |
| ND6- | G | 0.00% | 0.00% | 0.01% | 0.00% | 0.00% | 0.01% | 0.01% | 99.85% | 0.01% | 0.00% | 0.00% | 0.00% |
| DdCBE_ | | | | | | | | | | | | | |
| replicate3 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND6-DdCBE

| Batch | Nucleotide | C | A | A | C | C | C | C | T | G | A | C | C |
|---|---|---|---|---|---|---|---|---|---|---|---|---|---|
| ND6- | A | 0.01% | 99.96% | 99.95% | 0.01% | 0.02% | 0.01% | 0.01% | 0.01% | 0.11% | 99.98% | 0.01% | 0.01% |
| DdCBE_ | | | | | | | | | | | | | |
| replicate1 | | | | | | | | | | | | | |
| ND6- | T | 0.25% | 0.00% | 0.00% | 0.01% | 0.01% | 0.00% | 0.01% | 99.98% | 0.02% | 0.01% | 0.01% | 0.01% |
| DdCBE_ | | | | | | | | | | | | | |
| replicate1 | | | | | | | | | | | | | |
| ND6- | C | 99.74% | 0.03% | 0.03% | 99.98% | 99.97% | 99.99% | 99.98% | 0.02% | 0.00% | 0.00% | 99.98% | 99.98% |
| DdCBE_ | | | | | | | | | | | | | |
| replicate1 | | | | | | | | | | | | | |
| ND6- | G | 0.00% | 0.01% | 0.02% | 0.00% | 0.00% | 0.00% | 0.00% | 0.00% | 99.87% | 0.01% | 0.00% | 0.00% |
| DdCBE_ | | | | | | | | | | | | | |
| replicate1 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | — | 0.00% | 0.00% | 0.00% | 0.00% | 0.00% | 0.00% | 0.01% | 0.00% | 0.00% | 0.00% | 0.00% | 0.00% |
| DdCBE_ | | | | | | | | | | | | | |
| replicate1 | | | | | | | | | | | | | |
| ND6- | A | 0.00% | 99.96% | 99.96% | 0.00% | 0.01% | 0.01% | 0.01% | 0.01% | 0.14% | 99.97% | 0.01% | 0.00% |
| DdCBE_ | | | | | | | | | | | | | |
| replicate2 | | | | | | | | | | | | | |
| ND6- | T | 0.19% | 0.00% | 0.00% | 0.00% | 0.00% | 0.01% | 0.01% | 99.97% | 0.01% | 0.01% | 0.01% | 0.01% |
| DdCBE_ | | | | | | | | | | | | | |
| replicate2 | | | | | | | | | | | | | |
| ND6- | C | 99.80% | 0.02% | 0.02% | 99.99% | 99.98% | 99.98% | 99.98% | 0.02% | 0.00% | 0.01% | 99.98% | 99.98% |
| DdCBE_ | | | | | | | | | | | | | |
| replicate2 | | | | | | | | | | | | | |
| ND6- | G | 0.00% | 0.01% | 0.02% | 0.00% | 0.00% | 0.00% | 0.00% | 0.00% | 99.84% | 0.01% | 0.00% | 0.00% |
| DdCBE_ | | | | | | | | | | | | | |
| replicate2 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | A | 0.00% | 99.97% | 99.96% | 0.01% | 0.01% | 0.01% | 0.01% | 0.00% | 0.07% | 99.98% | 0.01% | 0.00% |
| DdCBE_ | | | | | | | | | | | | | |
| replicate3 | | | | | | | | | | | | | |
| ND6- | T | 0.18% | 0.00% | 0.00% | 0.01% | 0.01% | 0.01% | 0.01% | 99.97% | 0.01% | 0.01% | 0.01% | 0.01% |
| DdCBE_ | | | | | | | | | | | | | |
| replicate3 | | | | | | | | | | | | | |
| ND6- | C | 99.81% | 0.02% | 0.02% | 99.98% | 99.98% | 99.99% | 99.98% | 0.02% | 0.00% | 0.01% | 99.98% | 99.99% |
| DdCBE_ | | | | | | | | | | | | | |
| replicate3 | | | | | | | | | | | | | |
| ND6- | G | 0.00% | 0.01% | 0.01% | 0.00% | 0.00% | 0.00% | 0.00% | 0.00% | 99.92% | 0.01% | 0.00% | 0.00% |
| DdCBE_ | | | | | | | | | | | | | |
| replicate3 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND6-DdCBE

| Batch | Nucleotide | C | C | C | A | T | G | C | C | T | C | A | G |
|---|---|---|---|---|---|---|---|---|---|---|---|---|---|
| ND6- | A | 0.01% | 0.02% | 0.00% | 99.97% | 0.00% | 0.02% | 0.01% | 0.00% | 0.01% | 0.00% | 99.96% | 30.47% |
| DdCBE_ | | | | | | | | | | | | | |
| replicate1 | | | | | | | | | | | | | |
| ND6- | T | 0.01% | 0.02% | 0.01% | 0.00% | 99.99% | 0.00% | 0.03% | 0.00% | 99.98% | 0.17% | 0.00% | 0.01% |
| DdCBE_ | | | | | | | | | | | | | |
| replicate1 | | | | | | | | | | | | | |
| ND6- | C | 99.98% | 99.96% | 99.97% | 0.01% | 0.01% | 0.01% | 99.96% | 99.99% | 0.01% | 99.82% | 0.02% | 0.03% |
| DdCBE_ | | | | | | | | | | | | | |
| replicate1 | | | | | | | | | | | | | |
| ND6- | G | 0.01% | 0.00% | 0.00% | 0.01% | 0.00% | 99.97% | 0.00% | 0.00% | 0.00% | 0.00% | 0.02% | 69.49% |
| DdCBE_ | | | | | | | | | | | | | |
| replicate1 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | — | 0.00% | 0.00% | 0.02% | 0.00% | 0.00% | 0.00% | 0.00% | 0.00% | 0.00% | 0.00% | 0.01% | 0.00% |
| DdCBE_ | | | | | | | | | | | | | |
| replicate1 | | | | | | | | | | | | | |
| ND6- | A | 0.00% | 0.03% | 0.00% | 99.95% | 0.01% | 0.01% | 0.03% | 0.00% | 0.01% | 0.00% | 99.97% | 29.83% |
| DdCBE_ | | | | | | | | | | | | | |
| replicate2 | | | | | | | | | | | | | |
| ND6- | T | 0.01% | 0.02% | 0.01% | 0.00% | 99.98% | 0.00% | 0.01% | 0.01% | 99.96% | 0.20% | 0.00% | 0.01% |
| DdCBE_ | | | | | | | | | | | | | |
| replicate2 | | | | | | | | | | | | | |
| ND6- | C | 99.99% | 99.96% | 99.97% | 0.03% | 0.01% | 0.01% | 99.96% | 99.98% | 0.02% | 99.79% | 0.01% | 0.04% |
| DdCBE_ | | | | | | | | | | | | | |
| replicate2 | | | | | | | | | | | | | |
| ND6- | G | 0.00% | 0.00% | 0.00% | 0.01% | 0.00% | 99.97% | 0.00% | 0.00% | 0.01% | 0.00% | 0.01% | 70.13% |
| DdCBE_ | | | | | | | | | | | | | |
| replicate2 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | | 0.00% | 0.00% | 0.01% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | A | 0.00% | 0.02% | 0.01% | 99.96% | 0.01% | 0.01% | 0.02% | 0.01% | 0.00% | 0.01% | 99.97% | 26.86% |
| DdCBE_ | | | | | | | | | | | | | |
| replicate3 | | | | | | | | | | | | | |
| ND6- | T | 0.01% | 0.01% | 0.01% | 0.01% | 99.98% | 0.01% | 0.02% | 0.01% | 99.96% | 0.15% | 0.01% | 0.01% |
| DdCBE_ | | | | | | | | | | | | | |
| replicate3 | | | | | | | | | | | | | |
| ND6- | C | 99.99% | 99.97% | 99.96% | 0.02% | 0.01% | 0.01% | 99.96% | 99.99% | 0.03% | 99.84% | 0.01% | 0.03% |
| DdCBE_ | | | | | | | | | | | | | |
| replicate3 | | | | | | | | | | | | | |
| ND6- | G | 0.00% | 0.00% | 0.00% | 0.01% | 0.00% | 99.97% | 0.00% | 0.00% | 0.01% | 0.00% | 0.02% | C |
| DdCBE_ | | | | | | | | | | | | | |
| replicate3 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND6- | — | 0.00% | 0.00% | 0.02% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND6-DdCBE

| Batch | Nucleotide | G | A | T | A | C | T | C | C | T | C | A | A |
|---|---|---|---|---|---|---|---|---|---|---|---|---|---|
| ND6- | A | 36.39% | 99.91% | 0.00% | 99.98% | 0.02% | 0.02% | 0.02% | 0.01% | 0.01% | 0.00% | 99.97% | 99.97% |
| DdCBE_ | | | | | | | | | | | | | |
| replicate1 | | | | | | | | | | | | | |
| ND6- | T | 0.01% | 0.01% | 99.99% | 0.01% | 5.37% | 99.97% | 18.62% | 0.45% | 99.96% | 0.11% | 0.00% | 0.00% |
| DdCBE_ | | | | | | | | | | | | | |
| replicate1 | | | | | | | | | | | | | |
| ND6- | C | 0.00% | 0.08% | 0.01% | 0.01% | 94.59% | 0.01% | 81.35% | 99.54% | 0.03% | 99.88% | 0.02% | 0.02% |
| DdCBE_ | | | | | | | | | | | | | |
| replicate1 | | | | | | | | | | | | | |
| ND6- | G | 63.59% | 0.00% | 0.00% | 0.00% | 0.00% | 0.00% | 0.00% | 0.00% | 0.00% | 0.01% | 0.01% | 0.01% |
| DdCBE_ | | | | | | | | | | | | | |
| replicate1 | | | | | | | | | | | | | |
| ND6- | N | 0.00% | 0.00% | 0.00% | 0.00% | 0.01% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | A | 35.45% | 99.90% | 0.00% | 99.96% | 0.03% | 0.01% | 0.02% | 0.01% | 0.00% | 0.00% | 99.97% | 99.96% |
| DdCBE_ | | | | | | | | | | | | | |
| replicate2 | | | | | | | | | | | | | |
| ND6- | T | 0.01% | 0.00% | 99.99% | 0.00% | 5.04% | 99.97% | 17.88% | 0.41% | 99.97% | 0.10% | 0.00% | 0.01% |
| DdCBE_ | | | | | | | | | | | | | |
| replicate2 | | | | | | | | | | | | | |
| ND6- | C | 0.00% | 0.07% | 0.01% | 0.03% | 94.92% | 0.01% | 82.09% | 99.58% | 0.02% | 99.89% | 0.02% | 0.02% |
| DdCBE_ | | | | | | | | | | | | | |
| replicate2 | | | | | | | | | | | | | |
| ND6- | G | 64.53% | 0.02% | 0.00% | 0.01% | 0.00% | 0.00% | 0.01% | 0.00% | 0.01% | 0.00% | 0.01% | 0.01% |
| DdCBE_ | | | | | | | | | | | | | |
| replicate2 | | | | | | | | | | | | | |
| ND6- | N | 0.00% | 0.00% | 0.00% | 0.00% | 0.01% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | A | 31.97% | 99.89% | 0.01% | 99.96% | 0.03% | 0.02% | 0.02% | 0.00% | 0.01% | 0.00% | 99.97% | 99.96% |
| DdCBE_ | | | | | | | | | | | | | |
| replicate3 | | | | | | | | | | | | | |
| ND6- | T | 0.01% | 0.01% | 99.97% | 0.01% | 4.50% | 99.96% | 15.39% | 0.34% | 99.98% | 0.08% | 0.00% | 0.01% |
| DdCBE_ | | | | | | | | | | | | | |
| replicate3 | | | | | | | | | | | | | |
| ND6- | C | 0.00% | 0.09% | 0.02% | 0.03% | 95.46% | 0.02% | 84.59% | 99.65% | 0.01% | 99.91% | 0.01% | 0.02% |
| DdCBE_ | | | | | | | | | | | | | |
| replicate3 | | | | | | | | | | | | | |
| ND6- | G | 68.02% | 0.02% | 0.00% | 0.01% | 0.00% | 0.00% | 0.00% | 0.00% | 0.00% | 0.00% | 0.01% | 0.01% |
| DdCBE_ | | | | | | | | | | | | | |
| replicate3 | | | | | | | | | | | | | |
| ND6- | N | 0.00% | 0.00% | 0.00% | 0.00% | 0.01% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND6-DdCBE

| Batch | Nucleotide | T | A | G | C | C | A | T | C | G | C | T | G |
|---|---|---|---|---|---|---|---|---|---|---|---|---|---|
| ND6- | A | 0.01% | 99.95% | 0.02% | 0.01% | 0.02% | 99.96% | 0.01% | 0.07% | 0.02% | 0.01% | 0.00% | 0.01% |
| DdCBE_ | | | | | | | | | | | | | |
| replicate1 | | | | | | | | | | | | | |
| ND6- | T | 99.97% | 0.01% | 0.02% | 0.01% | 0.02% | 0.01% | 99.98% | 0.25% | 0.03% | 0.01% | 99.97% | 0.02% |
| DdCBE_ | | | | | | | | | | | | | |
| replicate | | | | | | | | | | | | | |
| ND6- | C | 0.01% | 0.03% | 0.01% | 99.99% | 99.96% | 0.02% | 0.01% | 99.69% | 0.01% | 99.98% | 0.03% | 0.01% |
| DdCBE_ | | | | | | | | | | | | | |
| replicate1 | | | | | | | | | | | | | |
| ND6- | G | 0.02% | 0.01% | 99.96% | 0.00% | 0.01% | 0.01% | 0.00% | 0.00% | 99.94% | 0.00% | 0.00% | 99.97% |
| DdCBE_ | | | | | | | | | | | | | |
| replicate1 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | A | 0.01% | 99.97% | 0.03% | 0.00% | 0.02% | 99.98% | 0.01% | 0.07% | 0.01% | 0.01% | 0.00% | 0.01% |
| DdCBE_ | | | | | | | | | | | | | |
| replicate2 | | | | | | | | | | | | | |
| ND6- | T | 99.96% | 0.01% | 0.02% | 0.01% | 0.02% | 0.00% | 99.97% | 0.23% | 0.02% | 0.01% | 99.97% | 0.02% |
| DdCBE_ | | | | | | | | | | | | | |
| replicate2 | | | | | | | | | | | | | |
| ND6- | C | 0.01% | 0.02% | 0.02% | 99.98% | 99.96% | 0.01% | 0.02% | 99.71% | 0.01% | 99.98% | 0.02% | 0.00% |
| DdCBE_ | | | | | | | | | | | | | |
| replicate2 | | | | | | | | | | | | | |
| ND6- | G | 0.01% | 0.01% | 99.94% | 0.00% | 0.00% | 0.01% | 0.00% | 0.00% | 99.96% | 0.00% | 0.01% | 99.97% |
| DdCBE_ | | | | | | | | | | | | | |
| replicate2 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | A | 0.01% | 99.97% | 0.02% | 0.01% | 0.03% | 99.96% | 0.01% | 0.07% | 0.02% | 0.00% | 0.01% | 0.02% |
| DdCBE_ | | | | | | | | | | | | | |
| replicate3 | | | | | | | | | | | | | |
| ND6- | T | 99.98% | 0.00% | 0.02% | 0.01% | 0.01% | 0.01% | 99.97% | 0.20% | 0.02% | 0.01% | 99.98% | 0.02% |
| DdCBE_ | | | | | | | | | | | | | |
| replicate3 | | | | | | | | | | | | | |
| ND6- | C | 0.01% | 0.02% | 0.01% | 99.98% | 99.96% | 0.01% | 0.02% | 99.72% | 0.01% | 99.99% | 0.01% | 0.00% |
| DdCBE_ | | | | | | | | | | | | | |
| replicate3 | | | | | | | | | | | | | |
| ND6- | G | 0.01% | 0.01% | 99.95% | 0.00% | 0.00% | 0.01% | 0.00% | 0.00% | 99.95% | 0.00% | 0.00% | 99.96% |
| DdCBE_ | | | | | | | | | | | | | |
| replicate3 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND6-DdCBE

| Batch | Nucleotide | T | A | G | T | A | T | A | T | C | C | A | A |
|---|---|---|---|---|---|---|---|---|---|---|---|---|---|
| ND6- | A | 0.01% | 99.95% | 0.01% | 0.03% | 99.97% | 0.01% | 99.98% | 0.02% | 0.01% | 0.00% | 99.97% | 99.98% |
| DdCBE_ | | | | | | | | | | | | | |
| replicate1 | | | | | | | | | | | | | |
| ND6- | T | 99.86% | 0.01% | 0.02% | 99.74% | 0.01% | 99.98% | 0.02% | 99.97% | 1.23% | 0.02% | 0.00% | 0.01% |
| DdCBE_ | | | | | | | | | | | | | |
| replicate | | | | | | | | | | | | | |
| ND6- | C | 0.12% | 0.02% | 0.01% | 0.01% | 0.01% | 0.01% | 0.00% | 0.01% | 98.75% | 99.98% | 0.02% | 0.00% |
| DdCBE_ | | | | | | | | | | | | | |
| replicate1 | | | | | | | | | | | | | |
| ND6- | G | 0.01% | 0.02% | 99.96% | 0.22% | 0.01% | 0.00% | 0.01% | 0.00% | 0.01% | 0.00% | 0.01% | 0.01% |
| DdCBE_ | | | | | | | | | | | | | |
| replicate1 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND6- | — | 0.00% | 0.00% | 0.00% | 0.00% | 0.00% | 0.00% | 0.00% | 0.00% | 0.00% | 0.01% | 0.00% | 0.00% |
| DdCBE_ | | | | | | | | | | | | | |
| replicate1 | | | | | | | | | | | | | |
| ND6- | A | 0.02% | 99.96% | 0.02% | 0.03% | 99.98% | 0.01% | 99.98% | 0.02% | 0.01% | 0.01% | 99.95% | 99.98% |
| DdCBE_ | | | | | | | | | | | | | |
| replicate2 | | | | | | | | | | | | | |
| ND6- | T | 99.86% | 0.01% | 0.02% | 99.75% | 0.00% | 99.98% | 0.01% | 99.96% | 1.21% | 0.03% | 0.00% | 0.01% |
| DdCBE_ | | | | | | | | | | | | | |
| replicate2 | | | | | | | | | | | | | |
| ND6- | C | 0.11% | 0.01% | 0.00% | 0.02% | 0.01% | 0.01% | 0.00% | 0.02% | 98.78% | 99.96% | 0.02% | 0.01% |
| DdCBE_ | | | | | | | | | | | | | |
| replicate2 | | | | | | | | | | | | | |
| ND6- | G | 0.01% | 0.02% | 99.96% | 0.20% | 0.01% | 0.00% | 0.01% | 0.00% | 0.00% | 0.00% | 0.02% | 0.01% |
| DdCBE_ | | | | | | | | | | | | | |
| replicate2 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND6- | A | 0.01% | 99.96% | 0.01% | 0.02% | 99.97% | 0.00% | 99.98% | 0.01% | 0.01% | 0.01% | 99.97% | 99.99% |
| DdCBE_ | | | | | | | | | | | | | |
| replicate3 | | | | | | | | | | | | | |
| ND6- | T | 99.91% | 0.01% | 0.03% | 99.76% | 0.01% | 99.99% | 0.01% | 99.97% | 0.98% | 0.02% | 0.01% | 0.00% |
| DdCBE_ | | | | | | | | | | | | | |
| replicate3 | | | | | | | | | | | | | |
| ND6- | C | 0.08% | 0.02% | 0.00% | 0.02% | 0.01% | 0.00% | 0.01% | 0.01% | 99.01% | 99.9% | 0.01% | 0.00% |
| DdCBE_ | | | | | | | | | | | | | |
| replicate3 | | | | | | | | | | | | | |
| ND6- | G | 0.00% | 0.02% | 99.96% | 0.21% | 0.01% | 0.00% | 0.00% | 0.00% | 0.00% | 0.00% | 0.01% | 0.00% |
| DdCBE_ | | | | | | | | | | | | | |
| replicate3 | | | | | | | | | | | | | |
| ND6- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND6- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND6-DdCBE

| Batch | Nucleotide | A | G | A | C | A |
|---|---|---|---|---|---|---|
| ND6- | A | 99.98% | 0.16% | 99.89% | 0.01% | 99.96% |
| DdCBE_ | | | | | | |
| replicate1 | | | | | | |
| ND6- | T | 0.01% | 0.01% | 0.01% | 0.02% | 0.00% |
| DdCBE_ | | | | | | |
| replicate1 | | | | | | |
| ND6- | C | 0.01% | 0.01% | 0.09% | 99.97% | 0.02% |
| DdCBE_ | | | | | | |
| replicate1 | | | | | | |
| ND6- | G | 0.00% | 99.82% | 0.01% | 0.00% | 0.01% |
| DdCBE_ | | | | | | |
| replicate1 | | | | | | |
| ND6- | N | 0.00% | 0.00% | 0.00% | 0.00% | 0.00% |
| DdCBE_ | | | | | | |
| replicate1 | | | | | | |
| ND6- | — | 0.01% | 0.00% | 0.00% | 0.00% | 0.00% |
| DdCBE_ | | | | | | |
| replicate1 | | | | | | |
| ND6- | A | 99.98% | 0.17% | 99.89% | 0.01% | 99.97% |
| DdCBE_ | | | | | | |
| replicate2 | | | | | | |
| ND6- | T | 0.00% | 0.01% | 0.01% | 0.01% | 0.00% |
| DdCBE_ | | | | | | |
| replicate2 | | | | | | |
| ND6- | C | 0.01% | 0.00% | 0.09% | 99.98% | 0.01% |
| DdCBE_ | | | | | | |
| replicate2 | | | | | | |
| ND6- | G | 0.00% | 99.82% | 0.01% | 0.00% | 0.01% |
| DdCBE_ | | | | | | |
| replicate2 | | | | | | |
| ND6- | N | 0.00% | 0.00% | 0.00% | 0.00% | 0.00% |
| DdCBE_ | | | | | | |
| replicate2 | | | | | | |
| ND6- | — | 0.01% | 0.00% | 0.00% | 0.00% | 0.00% |
| DdCBE_ | | | | | | |
| replicate2 | | | | | | |
| ND6- | A | 99.95% | 0.14% | 99.92% | 0.01% | 99.97% |
| DdCBE_ | | | | | | |
| replicate3 | | | | | | |
| ND6- | T | 0.01% | 0.02% | 0.01% | 0.01% | 0.01% |
| DdCBE_ | | | | | | |
| replicate3 | | | | | | |
| ND6- | C | 0.02% | 0.00% | 0.06% | 99.98% | 0.01% |
| DdCBE_ | | | | | | |
| replicate3 | | | | | | |
| ND6- | G | 0.01% | 99.84% | 0.01% | 0.00% | 0.01% |
| DdCBE_ | | | | | | |
| replicate3 | | | | | | |
| ND6- | N | 0.00% | 0.00% | 0.00% | 0.00% | 0.00% |
| DdCBE_ | | | | | | |
| replicate3 | | | | | | |
| ND6- | — | 0.01% | 0.00% | 0.00% | 0.00% | 0.00% |
| DdCBE_ | | | | | | |
| replicate3 | | | | | | |

TABLE 12B
Base Percentages at TALE Binding Sites-ND5.1-DdCBE

| | Nu- | | | | | | | | | | | | |
|---|---|---|---|---|---|---|---|---|---|---|---|---|---|
| | cle- | | | | | | | | | | | | |
| Batch | otide | C | T | C | C | C | T | C | A | C | C | A | T |
| ND5.1- | A | 0.01% | 0.00% | 0.05% | 0.02% | 0.03% | 0.02% | 0.04% | 99.95% | 0.02% | 0.02% | 99.98% | 0.03% |
| DdCBE_ | | | | | | | | | | | | | |
| replicate 1 | | | | | | | | | | | | | |
| ND5.1- | T | 0.01% | 99.98% | 0.14% | 0.03% | 0.01% | 99.96% | 0.12% | 0.02% | 0.02% | 0.03% | 0.00% | 99.97% |
| DdCBE_ | | | | | | | | | | | | | |
| replicate1 | | | | | | | | | | | | | |
| ND5.1- | C | 99.97% | 0.01% | 99.81% | 99.95% | 99.93% | 0.01% | 99.84% | 0.00% | 99.96% | 99.95% | 0.00% | 0.00% |
| DdCBE_ | | | | | | | | | | | | | |
| replicate1 | | | | | | | | | | | | | |
| ND5.1- | G | 0.00% | 0.00% | 0.00% | 0.00% | 0.01% | 0.00% | 0.00% | 0.03% | 0.00% | 0.00% | 0.01% | 0.00% |
| DdCBE_ | | | | | | | | | | | | | |
| replicate1 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | — | 0.00% | 0.00% | 0.00% | 0.00% | 0.02% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | A | 0.01% | 0.01% | 0.06% | 0.01% | 0.04% | 0.01% | 0.01% | 99.95% | 0.01% | 0.01% | 99.96% | 0.01% |
| DdCBE_ | | | | | | | | | | | | | |
| replicate2 | | | | | | | | | | | | | |
| ND5.1- | T | 0.01% | 99.95% | 0.14% | 0.03% | 0.02% | 99.97% | 0.14% | 0.02% | 0.01% | 0.03% | 0.02% | 99.96% |
| DdCBE_ | | | | | | | | | | | | | |
| replicate2 | | | | | | | | | | | | | |
| ND5.1- | C | 99.98% | 0.04% | 99.79% | 99.96% | 99.92% | 0.02% | 99.84% | 0.01% | 99.97% | 99.96% | 0.00% | 0.03% |
| DdCBE_ | | | | | | | | | | | | | |
| replicate2 | | | | | | | | | | | | | |
| ND5.1- | G | 0.01% | 0.00% | 0.00% | 0.00% | 0.01% | 0.00% | 0.00% | 0.02% | 0.00% | 0.00% | 0.01% | 0.00% |
| DdCBE_ | | | | | | | | | | | | | |
| replicate2 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.1- | — | 0.00% | 0.00% | 0.00% | 0.00% | 0.02% | 0.00% | 0.00% | 0.01% | 0.01% | 0.00% | 0.00% | 0.00% |
| DdCBE_ | | | | | | | | | | | | | |
| replicate2 | | | | | | | | | | | | | |
| ND5.1- | A | 0.01% | 0.01% | 0.06% | 0.01% | 0.03% | 0.01% | 0.06% | 99.96% | 0.02% | 0.01% | 99.97% | 0.01% |
| DdCBE_ | | | | | | | | | | | | | |
| replicate3 | | | | | | | | | | | | | |
| ND5.1- | T | 0.02% | 99.96% | 0.09% | 0.03% | 0.02% | 99.97% | 0.08% | 0.02% | 0.04% | 0.03% | 0.02% | 99.98% |
| DdCBE_ | | | | | | | | | | | | | |
| replicate3 | | | | | | | | | | | | | |
| ND5.1- | C | 99.97% | 0.03% | 99.85% | 99.95% | 99.92% | 0.02% | 99.86% | 0.01% | 99.94% | 99.96% | 0.00% | 0.01% |
| DdCBE_ | | | | | | | | | | | | | |
| replicate3 | | | | | | | | | | | | | |
| ND5.1- | G | 0.00% | 0.00% | 0.00% | 0.01% | 0.00% | 0.00% | 0.00% | 0.02% | 0.00% | 0.00% | 0.00% | 0.00% |
| DdCBE_ | | | | | | | | | | | | | |
| replicate3 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.1- | — | 0.00% | 0.00% | 0.00% | 0.00% | 0.02% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.1-DdCBE

| | Nu- | | | | | | | | | | | | |
|---|---|---|---|---|---|---|---|---|---|---|---|---|---|
| | cle- | | | | | | | | | | | | |
| Batch | otide | T | G | G | C | A | G | C | C | T | A | G | C |
| ND5.1- | A | 0.03% | 0.05% | 0.02% | 0.00% | 99.97% | 0.15% | 0.01% | 0.01% | 0.01% | 99.98% | 0.01% | 0.01% |
| DdCBE_ | | | | | | | | | | | | | |
| replicate 1 | | | | | | | | | | | | | |
| ND5.1- | T | 99.96% | 0.03% | 0.03% | 0.04% | 0.00% | 0.01% | 0.03% | 0.01% | 99.99% | 0.01% | 0.01% | 0.01% |
| DdCBE_ | | | | | | | | | | | | | |
| replicate1 | | | | | | | | | | | | | |
| ND5.1- | C | 0.01% | 0.01% | 0.00% | 99.95% | 0.00% | 0.00% | 99.95% | 99.98% | 0.01% | 0.00% | 0.00% | 99.97% |
| DdCBE_ | | | | | | | | | | | | | |
| replicate1 | | | | | | | | | | | | | |
| ND5.1- | G | 0.00% | 99.91% | 99.9% | 0.00% | 0.02% | 99.83% | 0.01% | 0.00% | 0.00% | 0.01% | 99.97% | 0.00% |
| DdCBE_ | | | | | | | | | | | | | |
| replicate1 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | — | 0.00% | 0.00% | 0.02% | 0.00% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | A | 0.01% | 0.04% | 0.02% | 0.02% | 99.95% | 0.13% | 0.01% | 0.01% | 0.01% | 99.96% | 0.01% | 0.02% |
| DdCBE_ | | | | | | | | | | | | | |
| replicate2 | | | | | | | | | | | | | |
| ND5.1- | T | 99.99% | 0.04% | 0.02% | 0.05% | 0.02% | 0.03% | 0.01% | 0.01% | 99.99% | 0.02% | 0.02% | 0.02% |
| DdCBE_ | | | | | | | | | | | | | |
| replicate2 | | | | | | | | | | | | | |
| ND5.1- | C | 0.00% | 0.01% | 0.00% | 99.92% | 0.00% | 0.00% | 99.97% | 99.97% | 0.01% | 0.00% | 0.00% | 99.97% |
| DdCBE_ | | | | | | | | | | | | | |
| replicate2 | | | | | | | | | | | | | |
| ND5.1- | G | 0.00% | 99.91% | 99.96% | 0.00% | 0.03% | 99.84% | 0.00% | 0.00% | 0.00% | 0.02% | 99.97% | 0.00% |
| DdCBE_ | | | | | | | | | | | | | |
| replicate2 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.1- | — | 0.00% | 0.00% | 0.01% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.1- | A | 0.03% | 0.05% | 0.02% | 0.01% | 99.93% | 0.18% | 0.01% | 0.02% | 0.01% | 99.96% | 0.01% | 0.02% |
| DdCBE_ | | | | | | | | | | | | | |
| replicate3 | | | | | | | | | | | | | |
| ND5.1- | T | 99.94% | 0.03% | 0.04% | 0.08% | 0.03% | 0.02% | 0.02% | 0.01% | 99.96% | 0.02% | 0.01% | 0.02% |
| DdCBE_ | | | | | | | | | | | | | |
| replicate3 | | | | | | | | | | | | | |
| ND5.1- | C | 0.03% | 0.00% | 0.00% | 99.90% | 0.00% | 0.00% | 99.97% | 99.97% | 0.02% | 0.00% | 0.01% | 99.96% |
| DdCBE_ | | | | | | | | | | | | | |
| replicate3 | | | | | | | | | | | | | |
| ND5.1- | G | 0.00% | 99.92% | 99.93% | 0.01% | 0.04% | 99.80% | 0.00% | 0.00% | 0.00% | 0.02% | 99.97% | 0.00% |
| DdCBE_ | | | | | | | | | | | | | |
| replicate3 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.1- | — | 0.00% | 0.00% | 0.01% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.1-DdCBE

| | Nu- | | | | | | | | | | | | |
|---|---|---|---|---|---|---|---|---|---|---|---|---|---|
| | cle- | | | | | | | | | | | | |
| Batch | otide | A | T | T | A | G | C | A | G | G | A | A | T |
| ND5.1- | A | 99.97% | 0.00% | 0.00% | 99.82% | 0.01% | 0.02% | 99.97% | 0.02% | 0.05% | 99.97% | 99.97% | 0.01% |
| DdCBE_ | | | | | | | | | | | | | |
| replicate 1 | | | | | | | | | | | | | |
| ND5.1- | T | 0.01% | 99.98% | 99.96% | 0.01% | 0.03% | 0.03% | 0.02% | 0.04% | 0.02% | 0.02% | 0.01% | 99.99% |
| DdCBE_ | | | | | | | | | | | | | |
| replicate1 | | | | | | | | | | | | | |
| ND5.1- | C | 0.01% | 0.02% | 0.02% | 0.00% | 0.00% | 99.95% | 0.00% | 0.01% | 0.00% | 0.00% | 0.00% | 0.00% |
| DdCBE_ | | | | | | | | | | | | | |
| replicate1 | | | | | | | | | | | | | |
| ND5.1- | G | 0.01% | 0.00% | 0.00% | 0.15% | 99.95% | 0.00% | 0.01% | 99.94% | 99.93% | 0.01% | 0.02% | 0.00% |
| DdCBE_ | | | | | | | | | | | | | |
| replicate1 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | — | 0.00% | 0.00% | 0.01% | 0.01% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | A | 99.96% | 0.01% | 0.00% | 99.87% | 0.03% | 0.01% | 99.96% | 0.01% | 0.03% | 99.98% | 99.97% | 0.01% |
| DdCBE_ | | | | | | | | | | | | | |
| replicate2 | | | | | | | | | | | | | |
| ND5.1- | T | 0.03% | 99.98% | 99.98% | 0.01% | 0.03% | 0.02% | 0.02% | 0.03% | 0.03% | 0.00% | 0.01% | 99.97% |
| DdCBE_ | | | | | | | | | | | | | |
| replicate2 | | | | | | | | | | | | | |
| ND5.1- | C | 0.00% | 0.01% | 0.02% | 0.00% | 0.00% | 99.97% | 0.01% | 0.01% | 0.00% | 0.01% | 0.00% | 0.02% |
| DdCBE_ | | | | | | | | | | | | | |
| replicate2 | | | | | | | | | | | | | |
| ND5.1- | G | 0.01% | 0.00% | 0.00% | 0.12% | 99.94% | 0.01% | 0.02% | 99.95% | 99.94% | 0.01% | 0.02% | 0.00% |
| DdCBE_ | | | | | | | | | | | | | |
| replicate2 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.1- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.1- | A | 99.98% | 0.01% | 0.01% | 99.79% | 0.02% | 0.01% | 99.94% | 0.01% | 0.03% | 99.94% | 99.95% | 0.01% |
| DdCBE_ | | | | | | | | | | | | | |
| replicate3 | | | | | | | | | | | | | |
| ND5.1- | T | 0.01% | 99.99% | 99.97% | 0.02% | 0.05% | 0.03% | 0.02% | 0.04% | 0.04% | 0.04% | 0.02% | 99.97% |
| DdCBE_ | | | | | | | | | | | | | |
| replicate3 | | | | | | | | | | | | | |
| ND5.1- | C | 0.00% | 0.01% | 0.02% | 0.00% | 0.01% | 99.96% | 0.01% | 0.00% | 0.01% | 0.01% | 0.00% | 0.02% |
| DdCBE_ | | | | | | | | | | | | | |
| replicate3 | | | | | | | | | | | | | |
| ND5.1- | G | 0.02% | 0.00% | 0.00% | 0.18% | 99.92% | 0.00% | 0.03% | 99.95% | 99.92% | 0.02% | 0.03% | 0.00% |
| DdCBE_ | | | | | | | | | | | | | |
| replicate3 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.1- | — | 0.00% | 0.00% | 0.00% | 0.01% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.1-DdCBE

| | Nu- | | | | | | | | | | | | |
|---|---|---|---|---|---|---|---|---|---|---|---|---|---|
| | cle- | | | | | | | | | | | | |
| Batch | otide | A | C | C | T | T | T | C | C | T | C | A | C |
| ND5.1- | A | 99.96% | 0.00% | 0.01% | 0.01% | 0.01% | 0.00% | 0.01% | 0.01% | 0.01% | 0.02% | 99.97% | 0.01% |
| DdCBE_ | | | | | | | | | | | | | |
| replicate 1 | | | | | | | | | | | | | |
| ND5.1- | T | 0.03% | 0.03% | 0.01% | 99.81% | 99.97% | 99.97% | 0.16% | 0.05% | 99.98% | 47.95% | 0.01% | 1.14% |
| DdCBE_ | | | | | | | | | | | | | |
| replicate1 | | | | | | | | | | | | | |
| ND5.1- | C | 0.00% | 99.96% | 99.97% | 0.18% | 0.03% | 0.03% | 99.82% | 99.94% | 0.01% | 52.03% | 0.00% | 98.85% |
| DdCBE_ | | | | | | | | | | | | | |
| replicate1 | | | | | | | | | | | | | |
| ND5.1- | G | 0.01% | 0.00% | 0.00% | 0.00% | 0.00% | 0.00% | 0.00% | 0.00% | 0.00% | 0.00% | 0.01% | 0.00% |
| DdCBE_ | | | | | | | | | | | | | |
| replicate1 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | A | 99.94% | 0.00% | 0.01% | 0.02% | 0.00% | 0.01% | 0.01% | 0.01% | 0.01% | 0.01% | 99.98% | 0.02% |
| DdCBE_ | | | | | | | | | | | | | |
| replicate2 | | | | | | | | | | | | | |
| ND5.1- | T | 0.03% | 0.03% | 0.02% | 99.84% | 99.97% | 99.94% | 0.24% | 0.04% | 99.96% | 47.91% | 0.00% | 1.36% |
| DdCBE_ | | | | | | | | | | | | | |
| replicate2 | | | | | | | | | | | | | |
| ND5.1- | C | 0.00% | 99.97% | 99.97% | 0.13% | 0.01% | 0.03% | 99.73% | 99.94% | 0.02% | 52.07% | 0.00% | 98.61% |
| DdCBE_ | | | | | | | | | | | | | |
| replicate2 | | | | | | | | | | | | | |
| ND5.1- | G | 0.03% | 0.00% | 0.00% | 0.01% | 0.01% | 0.00% | 0.01% | 0.00% | 0.00% | 0.00% | 0.01% | 0.00% |
| DdCBE_ | | | | | | | | | | | | | |
| replicate2 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.1- | — | 0.00% | 0.00% | 0.00% | 0.00% | 0.00% | 0.01% | 0.00% | 0.00% | 0.01% | 0.01% | 0.00% | 0.01% |
| DdCBE_ | | | | | | | | | | | | | |
| replicate2 | | | | | | | | | | | | | |
| ND5.1- | A | 99.95% | 0.01% | 0.02% | 0.01% | 0.01% | 0.02% | 0.01% | 0.02% | 0.01% | 0.02% | 99.95% | 0.02% |
| DdCBE_ | | | | | | | | | | | | | |
| replicate3 | | | | | | | | | | | | | |
| ND5.1- | T | 0.02% | 0.03% | 0.02% | 99.80% | 99.96% | 99.96% | 0.13% | 0.06% | 99.97% | 43.61% | 0.01% | 1.03% |
| DdCBE_ | | | | | | | | | | | | | |
| replicate3 | | | | | | | | | | | | | |
| ND5.1- | C | 0.01% | 99.96% | 99.96% | 0.19% | 0.03% | 0.02% | 99.86% | 99.92% | 0.02% | 56.36% | 0.01% | 98.95% |
| DdCBE_ | | | | | | | | | | | | | |
| replicate3 | | | | | | | | | | | | | |
| ND5.1- | G | 0.02% | 0.00% | 0.00% | 0.00% | 0.00% | 0.00% | 0.00% | 0.00% | 0.00% | 0.00% | 0.03% | 0.00% |
| DdCBE_ | | | | | | | | | | | | | |
| replicate3 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.1- | — | 0.00% | 0.00% | 0.00% | 0.00% | 0.00% | 0.00% | 0.00% | 0.00% | 0.00% | 0.01% | 0.00% | 0.01% |
| DdCBE_ | | | | | | | | | | | | | |
| replicate3 | | | | | | | | | | | | | |

Base Percentages at TALE Binding Sites-ND5.1-DdCBE

| | Nu- | | | | | | | | | | | | |
|---|---|---|---|---|---|---|---|---|---|---|---|---|---|
| | cle- | | | | | | | | | | | | |
| Batch | otide | A | G | G | T | T | T | C | T | A | C | T | C |
| ND5.1- | A | 99.97% | 0.00% | 0.02% | 0.02% | 0.00% | 0.00% | 0.01% | 0.01% | 99.95% | 0.01% | 0.01% | 0.01% |
| DdCBE_ | | | | | | | | | | | | | |
| replicate 1 | | | | | | | | | | | | | |
| ND5.1- | T | 0.01% | 0.02% | 0.01% | 99.82% | 99.97% | 99.99% | 0.03% | 99.98% | 0.02% | 0.04% | 99.97% | 0.03% |
| DdCBE_ | | | | | | | | | | | | | |
| replicate1 | | | | | | | | | | | | | |
| ND5.1- | C | 0.00% | 0.00% | 0.00% | 0.09% | 0.02% | 0.00% | 99.96% | 0.01% | 0.01% | 99.95% | 0.01% | 99.95% |
| DdCBE_ | | | | | | | | | | | | | |
| replicate1 | | | | | | | | | | | | | |
| ND5.1- | G | 0.02% | 99.98% | 99.96% | 0.07% | 0.00% | 0.00% | 0.00% | 0.00% | 0.02% | 0.00% | 0.00% | 0.01% |
| DdCBE_ | | | | | | | | | | | | | |
| replicate1 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate 1 | | | | | | | | | | | | | |
| ND5.1- | A | 99.96% | 0.00% | 0.01% | 0.02% | 0.00% | 0.01% | 0.02% | 0.01% | 99.97% | 0.01% | 0.00% | 0.01% |
| DdCBE_ | | | | | | | | | | | | | |
| replicate2 | | | | | | | | | | | | | |
| ND5.1- | T | 0.01% | 0.03% | 0.02% | 99.79% | 99.97% | 99.97% | 0.02% | 99.98% | 0.02% | 0.02% | 99.96% | 0.02% |
| DdCBE_ | | | | | | | | | | | | | |
| replicate2 | | | | | | | | | | | | | |
| ND5.1- | C | 0.01% | 0.00% | 0.00% | 0.12% | 0.02% | 0.01% | 99.96% | 0.01% | 0.00% | 99.97% | 0.04% | 99.96% |
| DdCBE_ | | | | | | | | | | | | | |
| replicate2 | | | | | | | | | | | | | |
| ND5.1- | G | 0.02% | 99.97% | 99.97% | 0.08% | 0.00% | 0.00% | 0.00% | 0.00% | 0.01% | 0.00% | 0.00% | 0.00% |
| DdCBE_ | | | | | | | | | | | | | |
| replicate2 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.1- | — | 0.01% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.1- | A | 99.96% | 0.01% | 0.01% | 0.02% | 0.01% | 0.02% | 0.01% | 0.01% | 99.96% | 0.01% | 0.02% | 0.02% |
| DdCBE_ | | | | | | | | | | | | | |
| replicate3 | | | | | | | | | | | | | |
| ND5.1- | T | 0.02% | 0.02% | 0.00% | 99.81% | 99.98% | 99.96% | 0.03% | 99.96% | 0.02% | 0.02% | 99.96% | 0.02% |
| DdCBE_ | | | | | | | | | | | | | |
| replicate3 | | | | | | | | | | | | | |
| ND5.1- | C | 0.00% | 0.00% | 0.00% | 0.12% | 0.01% | 0.02% | 99.96% | 0.02% | 0.01% | 99.96% | 0.02% | 99.96% |
| DdCBE_ | | | | | | | | | | | | | |
| replicate3 | | | | | | | | | | | | | |
| ND5.1- | G | 0.02% | 99.97% | 99.99% | 0.05% | 0.00% | 0.00% | 0.00% | 0.01% | 0.01% | 0.00% | 0.00% | 0.00% |
| DdCBE_ | | | | | | | | | | | | | |
| replicate3 | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.1- | — | 0.01% | 0.00% | 0.00% | 0.00% | 0.00% | 0.01% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.1-DdCBE

| | Nu- | | | | | | | | | | | | | |
|---|---|---|---|---|---|---|---|---|---|---|---|---|---|---|
| | cle- | | | | | | | | | | | | | |
| Batch | otide | C | A | A | A | G | A | C | C | A | C | A | T | C |
| ND5.1- | A | 0.08% | 99.95% | 99.98% | 99.94% | 0.03% | 99.98% | 0.02% | 0.03% | 99.95% | 0.01% | 99.98% | 0.03% | 0.03% |
| DdCBE_ | | | | | | | | | | | | | | |
| replicate 1 | | | | | | | | | | | | | | |
| ND5.1- | T | 0.03% | 0.01% | 0.01% | 0.00% | 0.04% | 0.01% | 0.02% | 0.02% | 0.01% | 0.03% | 0.01% | 99.95% | 0.14% |
| DdCBE_ | | | | | | | | | | | | | | |
| replicate1 | | | | | | | | | | | | | | |
| ND5.1- | C | 99.87% | 0.01% | 0.00% | 0.01% | 0.00% | 0.00% | 99.96% | 99.95% | 0.01% | 99.96% | 0.00% | 0.02% | 99.82% |
| DdCBE_ | | | | | | | | | | | | | | |
| replicate1 | | | | | | | | | | | | | | |
| ND5.1- | G | 0.01% | 0.03% | 0.01% | 0.03% | 99.94% | 0.01% | 0.00% | 0.00% | 0.03% | 0.01% | 0.00% | 0.00% | 0.00% |
| DdCBE_ | | | | | | | | | | | | | | |
| replicate1 | | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | | |
| replicate 1 | | | | | | | | | | | | | | |
| ND5.1- | — | 0.00% | 0.00% | 0.00% | 0.01% | 0.00% | 0.00% | 0.00% | 0.00% | 0.00% | 0.00% | 0.00% | 0.00% | 0.00% |
| DdCBE_ | | | | | | | | | | | | | | |
| replicate 1 | | | | | | | | | | | | | | |
| ND5.1- | A | 0.09% | 99.94% | 99.98% | 99.93% | 0.02% | 99.97% | 0.01% | 0.03% | 99.96% | 0.01% | 99.97% | 0.02% | 0.02% |
| DdCBE_ | | | | | | | | | | | | | | |
| replicate2 | | | | | | | | | | | | | | |
| ND5.1- | T | 0.03% | 0.01% | 0.01% | 0.00% | 0.03% | 0.01% | 0.01% | 0.01% | 0.03% | 0.02% | 0.01% | 99.96% | 0.17% |
| DdCBE_ | | | | | | | | | | | | | | |
| replicate2 | | | | | | | | | | | | | | |
| ND5.1- | C | 99.87% | 0.03% | 0.00% | 0.00% | 0.01% | 0.01% | 99.98% | 99.96% | 0.01% | 99.96% | 0.00% | 0.01% | 99.81% |
| DdCBE_ | | | | | | | | | | | | | | |
| replicate2 | | | | | | | | | | | | | | |
| ND5.1- | G | 0.01% | 0.02% | 0.01% | 0.03% | 99.94% | 0.01% | 0.00% | 0.01% | 0.01% | 0.00% | 0.02% | 0.00% | 0.00% |
| DdCBE_ | | | | | | | | | | | | | | |
| replicate2 | | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | | |
| replicate2 | | | | | | | | | | | | | | |
| ND5.1- | — | 0.01% | 0.00% | 0.00% | 0.04% | 0.00% | 0.00% | 0.00% | 0.00% | 0.00% | 0.01% | 0.01% | 0.00% | 0.00% |
| DdCBE_ | | | | | | | | | | | | | | |
| replicate2 | | | | | | | | | | | | | | |
| ND5.1- | A | 0.09% | 99.96% | 99.99% | 99.94% | 0.03% | 99.96% | 0.00% | 0.04% | 99.96% | 0.02% | 99.97% | 0.02% | 0.02% |
| DdCBE_ | | | | | | | | | | | | | | |
| replicate3 | | | | | | | | | | | | | | |
| ND5.1- | T | 0.03% | 0.00% | 0.00% | 0.01% | 0.01% | 0.03% | 0.01% | 0.02% | 0.03% | 0.01% | 0.01% | 99.96% | 0.11% |
| DdCBE_ | | | | | | | | | | | | | | |
| replicate3 | | | | | | | | | | | | | | |
| ND5.1- | C | 99.87% | 0.02% | 0.00% | 0.01% | 0.01% | 0.01% | 99.99% | 99.93% | 0.00% | 99.97% | 0.01% | 0.02% | 99.87% |
| DdCBE_ | | | | | | | | | | | | | | |
| replicate3 | | | | | | | | | | | | | | |
| ND5.1- | G | 0.01% | 0.02% | 0.01% | 0.03% | 99.95% | 0.01% | 0.00% | 0.00% | 0.02% | 0.00% | 0.01% | 0.00% | 0.00% |
| DdCBE_ | | | | | | | | | | | | | | |
| replicate3 | | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | | |
| replicate3 | | | | | | | | | | | | | | |
| ND5.1- | — | 0.00% | 0.00% | 0.00% | 0.02% | 0.00% | 0.00% | 0.00% | 0.01% | 0.00% | 0.00% | 0.00% | 0.00% | 0.00% |
| DdCBE_ | | | | | | | | | | | | | | |
| replicate3 | | | | | | | | | | | | | | |

Base Percentages at TALE Binding Sites-ND5.1-DdCBE

| | Nu- | | | | | | | | | | | | | |
|---|---|---|---|---|---|---|---|---|---|---|---|---|---|---|
| | cle- | | | | | | | | | | | | | |
| Batch | otide | A | T | C | G | A | A | A | C | C | G | C | A | A |
| ND5.1- | A | 99.97% | 0.01% | 0.01% | 0.05% | 99.95% | 99.98% | 99.98% | 0.01% | 0.01% | 0.05% | 0.01% | 99.96% | 99.98% |
| DdCBE_ | | | | | | | | | | | | | | |
| replicate 1 | | | | | | | | | | | | | | |
| ND5.1- | T | 0.01% | 99.97% | 0.12% | 0.01% | 0.01% | 0.00% | 0.01% | 0.01% | 0.01% | 0.03% | 0.03% | 0.00% | 0.00% |
| DdCBE_ | | | | | | | | | | | | | | |
| replicate1 | | | | | | | | | | | | | | |
| ND5.1- | C | 0.01% | 0.02% | 99.86% | 0.00% | 0.00% | 0.01% | 0.01% | 99.97% | 99.97% | 0.01% | 99.96% | 0.01% | 0.00% |
| DdCBE_ | | | | | | | | | | | | | | |
| replicate1 | | | | | | | | | | | | | | |
| ND5.1- | G | 0.01% | 0.00% | 0.01% | 99.93% | 0.03% | 0.01% | 0.00% | 0.00% | 0.00% | 99.91% | 0.00% | 0.03% | 0.01% |
| DdCBE_ | | | | | | | | | | | | | | |
| replicate1 | | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | | |
| replicate 1 | | | | | | | | | | | | | | |
| ND5.1- | — | 0.00% | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | | |
| replicate 1 | | | | | | | | | | | | | | |
| ND5.1- | A | 99.98% | 0.02% | 0.01% | 0.05% | 99.98% | 99.97% | 99.97% | 0.01% | 0.02% | 0.07% | 0.01% | 99.97% | 99.97% |
| DdCBE_ | | | | | | | | | | | | | | |
| replicate2 | | | | | | | | | | | | | | |
| ND5.1- | T | 0.01% | 99.97% | 0.12% | 0.03% | 0.01% | 0.00% | 0.01% | 0.01% | 0.02% | 0.02% | 0.03% | 0.01% | 0.01% |
| DdCBE_ | | | | | | | | | | | | | | |
| replicate2 | | | | | | | | | | | | | | |
| ND5.1- | C | 0.00% | 0.01% | 99.86% | 0.00% | 0.00% | 0.01% | 0.01% | 99.97% | 99.96% | 0.01% | 99.96% | 0.01% | 0.00% |
| DdCBE_ | | | | | | | | | | | | | | |
| replicate2 | | | | | | | | | | | | | | |
| ND5.1- | G | 0.01% | 0.00% | 0.01% | 99.92% | 0.01% | 0.02% | 0.02% | 0.00% | 0.00% | 99.90% | 0.00% | 0.01% | 0.02% |
| DdCBE_ | | | | | | | | | | | | | | |
| replicate2 | | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | | |
| replicate2 | | | | | | | | | | | | | | |
| ND5.1- | — | 0.00% | 0.00% | 0.00% | 0.00% | 0.00% | 0.00% | 0.01% | 0.00% | 0.00% | 0.00% | 0.00% | 0.00% | 0.00% |
| DdCBE_ | | | | | | | | | | | | | | |
| replicate2 | | | | | | | | | | | | | | |
| ND5.1- | A | 99.96% | 0.01% | 0.02% | 0.07% | 99.96% | 99.97% | 99.95% | 0.01% | 0.02% | 0.04% | 0.01% | 99.94% | 99.97% |
| DdCBE_ | | | | | | | | | | | | | | |
| replicate3 | | | | | | | | | | | | | | |
| ND5.1- | T | 0.02% | 99.98% | 0.14% | 0.02% | 0.02% | 0.01% | 0.01% | 0.02% | 0.02% | 0.02% | 0.02% | 0.02% | 0.01% |
| DdCBE_ | | | | | | | | | | | | | | |
| replicate3 | | | | | | | | | | | | | | |
| ND5.1- | C | 0.00% | 0.01% | 99.83% | 0.00% | 0.00% | 0.00% | 0.03% | 99.98% | 99.95% | 0.01% | 99.97% | 0.01% | 0.01% |
| DdCBE_ | | | | | | | | | | | | | | |
| replicate3 | | | | | | | | | | | | | | |
| ND5.1- | G | 0.02% | 0.00% | 0.01% | 99.90% | 0.01% | 0.02% | 0.01% | 0.00% | 0.01% | 99.93% | 0.01% | 0.04% | 0.01% |
| DdCBE_ | | | | | | | | | | | | | | |
| replicate3 | | | | | | | | | | | | | | |
| ND5.1- | N | 0.00% | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | | |
| replicate3 | | | | | | | | | | | | | | |
| ND5.1- | — | 0.00% | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | | |
| replicate3 | | | | | | | | | | | | | | |

TABLE 12C
Base Percentages at TALE Binding Sites-ND5.2-DdCBE

| Batch | Nucleotide | T | C | G | A | A | A | A | A | T | A | G | G |
|---|---|---|---|---|---|---|---|---|---|---|---|---|---|
| ND5.2- | A | 0.01% | 0.08% | 0.02% | 99.94% | 99.97% | 99.97% | 99.98% | 99.96% | 0.00% | 99.96% | 0.02% | 0.03% |
| DdCBE_ | | | | | | | | | | | | | |
| replicate1 | | | | | | | | | | | | | |
| ND5.2- | T | 99.96% | 0.02% | 0.02% | 0.00% | 0.01% | 0.01% | 0.01% | 0.01% | 99.98% | 0.02% | 0.03% | 0.03% |
| DdCBE_ | | | | | | | | | | | | | |
| replicate1 | | | | | | | | | | | | | |
| ND5.2- | C | 0.01% | 99.88% | 0.00% | 0.05% | 0.01% | 0.01% | 0.00% | 0.01% | 0.01% | 0.01% | 0.00% | 0.01% |
| DdCBE_ | | | | | | | | | | | | | |
| replicate 1 | | | | | | | | | | | | | |
| ND5.2- | G | 0.01% | 0.00% | 99.96% | 0.01% | 0.01% | 0.01% | 0.01% | 0.01% | 0.00% | 0.01% | 99.95% | 99.92% |
| DdCBE_ | | | | | | | | | | | | | |
| replicate1 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | — | 0.00% | 0.01% | 0.00% | 0.00% | 0.00% | 0.00% | 0.00% | 0.02% | 0.00% | 0.00% | 0.00% | 0.00% |
| DdCBE_ | | | | | | | | | | | | | |
| replicate1 | | | | | | | | | | | | | |
| ND5.2- | A | 0.00% | 0.06% | 0.03% | 99.94% | 99.87% | 99.94% | 99.90% | 99.87% | 0.00% | 99.94% | 0.00% | 0.03% |
| DdCBE_ | | | | | | | | | | | | | |
| replicate2 | | | | | | | | | | | | | |
| ND5.2- | T | 99.94% | 0.00% | 0.03% | 0.03% | 0.00% | 0.03% | 0.03% | 0.00% | 99.94% | 0.03% | 0.03% | 0.06% |
| DdCBE_ | | | | | | | | | | | | | |
| replicate2 | | | | | | | | | | | | | |
| ND5.2- | C | 0.06% | 99.94% | 0.00% | 0.03% | 0.06% | 0.03% | 0.03% | 0.03% | 0.06% | 0.03% | 0.00% | 0.03% |
| DdCBE_ | | | | | | | | | | | | | |
| replicate2 | | | | | | | | | | | | | |
| ND5.2- | G | 0.00% | 0.00% | 99.90% | 0.00% | 0.06% | 0.00% | 0.03% | 0.00% | 0.00% | 0.00% | 99.97% | 99.87% |
| DdCBE_ | | | | | | | | | | | | | |
| replicate2 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | | 0.00% | 0.00% | 0.03% | 0.00% | 0.00% | 0.00% | 0.00% | 0.10% | 0.00% | 0.00% | 0.00% | 0.00% |
| DdCBE_ | | | | | | | | | | | | | |
| replicate2 | | | | | | | | | | | | | |
| ND5.2- | A | 0.01% | 0.07% | 0.01% | 99.91% | 99.97% | 99.97% | 99.97% | 99.96% | 0.00% | 99.97% | 0.02% | 0.02% |
| DdCBE_ | | | | | | | | | | | | | |
| replicate3 | | | | | | | | | | | | | |
| ND5.2- | T | 99.97% | 0.02% | 0.02% | 0.00% | 0.01% | 0.01% | 0.01% | 0.01% | 99.99% | 0.01% | 0.03% | 0.02% |
| DdCBE_ | | | | | | | | | | | | | |
| replicate3 | | | | | | | | | | | | | |
| ND5.2- | C | 0.01% | 99.90% | 0.00% | 0.07% | 0.01% | 0.01% | 0.01% | 0.01% | 0.01% | 0.01% | 0.00% | 0.01% |
| DdCBE_ | | | | | | | | | | | | | |
| replicate3 | | | | | | | | | | | | | |
| ND5.2- | G | 0.01% | 0.00% | 99.96% | 0.02% | 0.01% | 0.01% | 0.01% | 0.01% | 0.00% | 0.00% | 99.95% | 99.95% |
| DdCBE_ | | | | | | | | | | | | | |
| replicate3 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.2- | — | 0.00% | 0.01% | 0.00% | 0.00% | 0.00% | 0.00% | 0.00% | 0.01% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.2-DdCBE

| Batch | Nucleotide | A | G | G | A | C | T | A | C | T | C | A | A |
|---|---|---|---|---|---|---|---|---|---|---|---|---|---|
| ND5.2- | A | 99.94% | 0.01% | 0.03% | 99.91% | 0.09% | 0.01% | 99.96% | 0.03% | 0.01% | 0.03% | 99.96% | 99.96% |
| DdCBE_ | | | | | | | | | | | | | |
| replicate1 | | | | | | | | | | | | | |
| ND5.2- | T | 0.01% | 0.02% | 0.04% | 0.01% | 0.02% | 99.97% | 0.02% | 0.01% | 99.97% | 0.02% | 0.01% | 0.01% |
| DdCBE_ | | | | | | | | | | | | | |
| replicate1 | | | | | | | | | | | | | |
| ND5.2- | C | 0.03% | 0.00% | 0.01% | 0.05% | 99.89% | 0.01% | 0.01% | 99.96% | 0.01% | 99.95% | 0.02% | 0.03% |
| DdCBE_ | | | | | | | | | | | | | |
| replicate 1 | | | | | | | | | | | | | |
| ND5.2- | G | 0.02% | 99.96% | 99.91% | 0.03% | 0.00% | 0.01% | 0.01% | 0.00% | 0.00% | 0.01% | 0.01% | 0.00% |
| DdCBE_ | | | | | | | | | | | | | |
| replicate1 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | A | 99.84% | 0.00% | 0.00% | 99.87% | 0.03% | 0.00% | 99.90% | 0.03% | 0.00% | 0.00% | 99.97% | 99.97% |
| DdCBE_ | | | | | | | | | | | | | |
| replicate2 | | | | | | | | | | | | | |
| ND5.2- | T | 0.03% | 0.03% | 0.06% | 0.00% | 0.00% | 99.87% | 0.03% | 0.06% | 99.97% | 0.06% | 0.00% | 0.00% |
| DdCBE_ | | | | | | | | | | | | | |
| replicate2 | | | | | | | | | | | | | |
| ND5.2- | C | 0.10% | 0.00% | 0.00% | 0.06% | 99.97% | 0.03% | 0.06% | 99.90% | 0.03% | 99.94% | 0.03% | 0.03% |
| DdCBE_ | | | | | | | | | | | | | |
| replicate2 | | | | | | | | | | | | | |
| ND5.2- | G | 0.03% | 99.97% | 99.94% | 0.06% | 0.00% | 0.10% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | A | 99.95% | 0.01% | 0.03% | 99.92% | 0.09% | 0.01% | 99.96% | 0.04% | 0.01% | 0.02% | 99.96% | 99.95% |
| DdCBE_ | | | | | | | | | | | | | |
| replicate3 | | | | | | | | | | | | | |
| ND5.2- | T | 0.01% | 0.02% | 0.03% | 0.01% | 0.01% | 99.96% | 0.02% | 0.01% | 99.98% | 0.03% | 0.01% | 0.01% |
| DdCBE_ | | | | | | | | | | | | | |
| replicate3 | | | | | | | | | | | | | |
| ND5.2- | C | 0.03% | 0.00% | 0.00% | 0.05% | 99.90% | 0.01% | 0.02% | 99.95% | 0.01% | 99.94% | 0.02% | 0.03% |
| DdCBE_ | | | | | | | | | | | | | |
| replicate3 | | | | | | | | | | | | | |
| ND5.2- | G | 0.01% | 99.97% | 99.93% | 0.02% | 0.00% | 0.01% | 0.01% | 0.00% | 0.00% | 0.00% | 0.01% | 0.01% |
| DdCBE_ | | | | | | | | | | | | | |
| replicate3 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.2- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.2-DdCBE

| Batch | Nucleotide | A | A | C | C | A | T | A | C | C | T | C | T |
|---|---|---|---|---|---|---|---|---|---|---|---|---|---|
| ND5.2- | A | 99.94% | 99.95% | 0.17% | 0.45% | 99.93% | 0.02% | 99.91% | 0.41% | 0.22% | 0.03% | 0.11% | 0.02% |
| DdCBE_ | | | | | | | | | | | | | |
| replicate1 | | | | | | | | | | | | | |
| ND5.2- | T | 0.01% | 0.02% | 0.11% | 0.13% | 0.01% | 99.96% | 0.02% | 0.61% | 0.15% | 99.94% | 0.03% | 99.95% |
| DdCBE_ | | | | | | | | | | | | | |
| replicate1 | | | | | | | | | | | | | |
| ND5.2- | C | 0.04% | 0.02% | 99.71% | 99.41% | 0.04% | 0.02% | 0.04% | 98.96% | 99.61% | 0.01% | 99.85% | 0.02% |
| DdCBE_ | | | | | | | | | | | | | |
| replicate 1 | | | | | | | | | | | | | |
| ND5.2- | G | 0.02% | 0.01% | 0.01% | 0.00% | 0.02% | 0.00% | 0.04% | 0.02% | 0.03% | 0.01% | 0.01% | 0.01% |
| DdCBE_ | | | | | | | | | | | | | |
| replicate1 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | A | 99.97% | 99.97% | 0.16% | 0.54% | 99.94% | 0.00% | 99.84% | 0.32% | 0.10% | 0.06% | 0.06% | 0.06% |
| DdCBE_ | | | | | | | | | | | | | |
| replicate2 | | | | | | | | | | | | | |
| ND5.2- | T | 0.00% | 0.00% | 0.06% | 0.10% | 0.00% | 99.97% | 0.03% | 0.48% | 0.13% | 99.90% | 0.03% | 99.90% |
| DdCBE_ | | | | | | | | | | | | | |
| replicate2 | | | | | | | | | | | | | |
| ND5.2- | C | 0.03% | 0.03% | 99.78% | 99.37% | 0.03% | 0.03% | 0.06% | 99.21% | 99.78% | 0.00% | 99.90% | 0.03% |
| DdCBE_ | | | | | | | | | | | | | |
| replicate2 | | | | | | | | | | | | | |
| ND5.2- | G | 0.00% | 0.00% | 0.00% | 0.00% | 0.03% | 0.00% | 0.06% | 0.00% | 0.00% | 0.03% | 0.00% | 0.00% |
| DdCBE_ | | | | | | | | | | | | | |
| replicate2 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | A | 99.95% | 99.97% | 0.16% | 0.40% | 99.95% | 0.01% | 99.94% | 0.38% | 0.19% | 0.04% | 0.12% | 0.02% |
| DdCBE_ | | | | | | | | | | | | | |
| replicate3 | | | | | | | | | | | | | |
| ND5.2- | T | 0.00% | 0.01% | 0.10% | 0.07% | 0.01% | 99.98% | 0.01% | 0.46% | 0.11% | 99.94% | 0.03% | 99.95% |
| DdCBE_ | | | | | | | | | | | | | |
| replicate3 | | | | | | | | | | | | | |
| ND5.2- | C | 0.02% | 0.01% | 99.74% | 99.53% | 0.03% | 0.01% | 0.02% | 99.14% | 99.67% | 0.01% | 99.84% | 0.02% |
| DdCBE_ | | | | | | | | | | | | | |
| replicate3 | | | | | | | | | | | | | |
| ND5.2- | G | 0.02% | 0.01% | 0.00% | 0.00% | 0.01% | 0.00% | 0.02% | 0.01% | 0.03% | 0.00% | 0.00% | 0.01% |
| DdCBE_ | | | | | | | | | | | | | |
| replicate3 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.2- | — | 0.00% | 0.01% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.2-DdCBE

| Batch | Nucleotide | C | A | C | T | T | C | A | A | C | C | T | C |
|---|---|---|---|---|---|---|---|---|---|---|---|---|---|
| ND5.2- | A | 0.04% | 99.95% | 0.07% | 0.01% | 0.01% | 0.15% | 99.95% | 99.95% | 0.04% | 0.04% | 0.01% | 0.05% |
| DdCBE_ | | | | | | | | | | | | | |
| replicate1 | | | | | | | | | | | | | |
| ND5.2- | T | 0.15% | 0.01% | 0.03% | 99.97% | 99.97% | 0.05% | 0.01% | 0.01% | 0.03% | 0.03% | 99.97% | 7.13% |
| DdCBE_ | | | | | | | | | | | | | |
| replicate1 | | | | | | | | | | | | | |
| ND5.2- | C | 99.81% | 0.03% | 99.89% | 0.02% | 0.02% | 99.78% | 0.02% | 0.02% | 99.85% | 99.91% | 0.01% | 92.81% |
| DdCBE_ | | | | | | | | | | | | | |
| replicate 1 | | | | | | | | | | | | | |
| ND5.2- | G | 0.01% | 0.01% | 0.00% | 0.00% | 0.01% | 0.01% | 0.01% | 0.01% | 0.08% | 0.01% | 0.01% | 0.01% |
| DdCBE_ | | | | | | | | | | | | | |
| replicate1 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | — | 0.00% | 0.00% | 0.00% | 0.00% | 0.00% | 0.01% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | A | 0.00% | 99.90% | 0.10% | 0.00% | 0.03% | 0.10% | 99.94% | 99.97% | 0.03% | 0.03% | 0.00% | 0.03% |
| DdCBE_ | | | | | | | | | | | | | |
| replicate2 | | | | | | | | | | | | | |
| ND5.2- | T | 0.10% | 0.00% | 0.03% | 99.97% | 99.90% | 0.13% | 0.00% | 0.00% | 0.03% | 0.00% | 99.97% | 6.92% |
| DdCBE_ | | | | | | | | | | | | | |
| replicate2 | | | | | | | | | | | | | |
| ND5.2- | C | 99.90% | 0.10% | 99.87% | 0.03% | 0.06% | 99.78% | 0.06% | 0.03% | 99.84% | 99.97% | 0.03% | 93.05% |
| DdCBE_ | | | | | | | | | | | | | |
| replicate2 | | | | | | | | | | | | | |
| ND5.2- | G | 0.00% | 0.00% | 0.00% | 0.00% | 0.00% | 0.00% | 0.00% | 0.00% | 0.10% | 0.00% | 0.00% | 0.00% |
| DdCBE_ | | | | | | | | | | | | | |
| replicate2 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | — | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | A | 0.04% | 99.95% | 0.07% | 0.01% | 0.01% | 0.16% | 99.95% | 99.95% | 0.04% | 0.05% | 0.02% | 0.05% |
| DdCBE_ | | | | | | | | | | | | | |
| replicate3 | | | | | | | | | | | | | |
| ND5.2- | T | 0.18% | 0.01% | 0.02% | 99.96% | 99.96% | 0.04% | 0.01% | 0.01% | 0.03% | 0.03% | 99.95% | 7.69% |
| DdCBE_ | | | | | | | | | | | | | |
| replicate3 | | | | | | | | | | | | | |
| ND5.2- | C | 99.77% | 0.03% | 99.90% | 0.02% | 0.01% | 99.78% | 0.01% | 0.02% | 99.85% | 99.91% | 0.02% | 92.26% |
| DdCBE_ | | | | | | | | | | | | | |
| replicate3 | | | | | | | | | | | | | |
| ND5.2- | G | 0.00% | 0.01% | 0.00% | 0.00% | 0.01% | 0.01% | 0.02% | 0.01% | 0.08% | 0.01% | 0.00% | 0.00% |
| DdCBE_ | | | | | | | | | | | | | |
| replicate3 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.2- | — | 0.00% | 0.00% | 0.00% | 0.00% | 0.00% | 0.01% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.2-DdCBE

| Batch | Nucleotide | C | C | T | C | A | C | C | A | T | T | G | G |
|---|---|---|---|---|---|---|---|---|---|---|---|---|---|
| ND5.2- | A | 0.07% | 0.04% | 0.02% | 0.07% | 99.97% | 0.03% | 0.02% | 99.97% | 0.00% | 0.01% | 0.03% | 0.01% |
| DdCBE_ | | | | | | | | | | | | | |
| replicate1 | | | | | | | | | | | | | |
| ND5.2- | T | 5.07% | 0.02% | 99.95% | 0.07% | 0.00% | 0.01% | 0.01% | 0.01% | 99.98% | 99.96% | 0.02% | 0.01% |
| DdCBE_ | | | | | | | | | | | | | |
| replicate1 | | | | | | | | | | | | | |
| ND5.2- | C | 94.85% | 99.93% | 0.01% | 99.85% | 0.01% | 99.95% | 99.96% | 0.01% | 0.01% | 0.01% | 0.00% | 0.01% |
| DdCBE_ | | | | | | | | | | | | | |
| replicate 1 | | | | | | | | | | | | | |
| ND5.2- | G | 0.00% | 0.00% | 0.01% | 0.00% | 0.01% | 0.00% | 0.00% | 0.01% | 0.01% | 0.01% | 99.95% | 99.96% |
| DdCBE_ | | | | | | | | | | | | | |
| replicate1 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | — | 0.00% | 0.02% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate1 | | | | | | | | | | | | | |
| ND5.2- | A | 0.13% | 0.00% | 0.06% | 0.10% | 100.00% | 0.00% | 0.00% | 100.00% | 0.00% | 0.00% | 0.03% | 0.00% |
| DdCBE_ | | | | | | | | | | | | | |
| replicate2 | | | | | | | | | | | | | |
| ND5.2- | T | 4.73% | 0.00% | 99.90% | 0.03% | 0.00% | 0.00% | 0.00% | 0.00% | 100.00% | 99.97% | 0.03% | 0.03% |
| DdCBE_ | | | | | | | | | | | | | |
| replicate2 | | | | | | | | | | | | | |
| ND5.2- | C | 95.14% | 100.00% | 0.00% | 99.84% | 0.00% | 100.00% | 100.00% | 0.00% | 0.00% | 0.03% | 0.00% | 0.00% |
| DdCBE_ | | | | | | | | | | | | | |
| replicate2 | | | | | | | | | | | | | |
| ND5.2- | G | 0.00% | 0.00% | 0.00% | 0.00% | 0.00% | 0.00% | 0.00% | 0.00% | 0.00% | 0.00% | 99.94% | 99.97% |
| DdCBE_ | | | | | | | | | | | | | |
| replicate2 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | | 0.00% | 0.00% | 0.03% | 0.03% | 0.00% | 0.00% | 0.00% | 0.00% | 0.00% | 0.00% | 0.00% | 0.00% |
| DdCBE_ | | | | | | | | | | | | | |
| replicate2 | | | | | | | | | | | | | |
| ND5.2- | A | 0.09% | 0.02% | 0.02% | 0.11% | 99.97% | 0.05% | 0.03% | 99.97% | 0.01% | 0.01% | 0.03% | 0.01% |
| DdCBE_ | | | | | | | | | | | | | |
| replicate3 | | | | | | | | | | | | | |
| ND5.2- | T | 5.57% | 0.02% | 99.97% | 0.05% | 0.01% | 0.01% | 0.02% | 0.01% | 99.97% | 99.96% | 0.02% | 0.02% |
| DdCBE_ | | | | | | | | | | | | | |
| replicate3 | | | | | | | | | | | | | |
| ND5.2- | C | 94.33% | 99.93% | 0.01% | 99.83% | 0.01% | 99.93% | 99.94% | 0.01% | 0.01% | 0.02% | 0.00% | 0.00% |
| DdCBE_ | | | | | | | | | | | | | |
| replicate3 | | | | | | | | | | | | | |
| ND5.2- | G | 0.00% | 0.00% | 0.00% | 0.00% | 0.01% | 0.00% | 0.00% | 0.01% | 0.00% | 0.00% | 99.94% | 99.96% |
| DdCBE_ | | | | | | | | | | | | | |
| replicate3 | | | | | | | | | | | | | |
| ND5.2- | N | 0.00% | 0.00% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |
| ND5.2- | — | 0.00% | 0.02% | 0.00% | 0.00% | 0.00% | 0.00% | 0.00% | 0.00% | 0.00% | 0.00% | 0.00% | 0.00% |
| DdCBE_ | | | | | | | | | | | | | |
| replicate3 | | | | | | | | | | | | | |

Base Percentages at TALE Binding Sites-ND5.2-DdCBE

| ND5.2- | A | C | A | G | C | C | T | A | G | C | A | T | T |
|---|---|---|---|---|---|---|---|---|---|---|---|---|---|
| DdCBE_ | | 0.04% | 99.97% | 0.14% | 0.03% | 0.03% | 0.01% | 99.96% | 0.01% | 0.04% | 99.97% | 0.00% | 0.01% |
| replicate1 | | | | | | | | | | | | | |
| ND5.2- | T | | | | | | | | | | | | |
| DdCBE_ | | 0.04% | 0.00% | 0.01% | 0.01% | 0.01% | 99.97% | 0.01% | 0.02% | 0.01% | 0.01% | 99.98% | 99.97% |
| replicate1 | | | | | | | | | | | | | |
| ND5.2- | C | | | | | | | | | | | | |
| DdCBE_ | | 99.92% | 0.01% | 0.00% | 99.95% | 99.95% | 0.01% | 0.01% | 0.00% | 99.94% | 0.01% | 0.01% | 0.01% |
| replicate 1 | | | | | | | | | | | | | |
| ND5.2- | G | | | | | | | | | | | | |
| DdCBE_ | | 0.00% | 0.01% | 99.84% | 0.00% | 0.00% | 0.00% | 0.01% | 99.97% | 0.00% | 0.01% | 0.00% | 0.01% |
| replicate1 | | | | | | | | | | | | | |
| ND5.2- | N | | | | | | | | | | | | |
| DdCBE_ | | 0.00% | 0.00% | 0.01% | 0.00% | 0.00% | 0.00% | 0.01% | 0.00% | 0.00% | 0.00% | 0.00% | 0.00% |
| replicate1 | | | | | | | | | | | | | |
| ND5.2- | — | | | | | | | | | | | | |
| DdCBE_ | | 0.00% | 0.00% | 0.00% | 0.00% | 0.00% | 0.00% | 0.00% | 0.00% | 0.00% | 0.00% | 0.00% | 0.01% |
| replicate1 | | | | | | | | | | | | | |
| ND5.2- | A | | | | | | | | | | | | |
| DdCBE_ | | 0.03% | 99.97% | 0.13% | 0.00% | 0.06% | 0.00% | 99.97% | 0.03% | 0.03% | 100.00% | 0.03% | 0.00% |
| replicate2 | | | | | | | | | | | | | |
| ND5.2- | T | | | | | | | | | | | | |
| DdCBE_ | | 0.00% | 0.00% | 0.03% | 0.03% | 0.06% | 99.97% | 0.03% | 0.00% | 0.03% | 0.00% | 99.97% | 100.00% |
| replicate2 | | | | | | | | | | | | | |
| ND5.2- | C | | | | | | | | | | | | |
| DdCBE_ | | 99.97% | 0.00% | 0.00% | 99.97% | 99.87% | 0.03% | 0.00% | 0.00% | 99.94% | 0.00% | 0.00% | 0.00% |
| replicate2 | | | | | | | | | | | | | |
| ND5.2- | G | | | | | | | | | | | | |
| DdCBE_ | | 0.00% | 0.03% | 99.84% | 0.00% | 0.00% | 0.00% | 0.00% | 99.97% | 0.00% | 0.00% | 0.00% | 0.00% |
| replicate2 | | | | | | | | | | | | | |
| ND5.2- | N | | | | | | | | | | | | |
| DdCBE_ | | 0.00% | 0.00% | 0.00% | 0.00% | 0.00% | 0.00% | 0.00% | 0.00% | 0.00% | 0.00% | 0.00% | 0.00% |
| replicate2 | | | | | | | | | | | | | |
| ND5.2- | | | | | | | | | | | | | |
| DdCBE_ | | 0.00% | 0.00% | 0.00% | 0.00% | 0.00% | 0.00% | 0.00% | 0.00% | 0.00% | 0.00% | 0.00% | 0.00% |
| replicate2 | | | | | | | | | | | | | |
| ND5.2- | A | | | | | | | | | | | | |
| DdCBE_ | | 0.04% | 99.97% | 0.17% | 0.02% | 0.03% | 0.01% | 99.96% | 0.01% | 0.04% | 99.98% | 0.01% | 0.01% |
| replicate3 | | | | | | | | | | | | | |
| ND5.2- | T | | | | | | | | | | | | |
| DdCBE_ | | 0.02% | 0.00% | 0.01% | 0.02% | 0.01% | 99.96% | 0.01% | 0.01% | 0.02% | 0.01% | 99.97% | 99.97% |
| replicate3 | | | | | | | | | | | | | |
| ND5.2- | C | | | | | | | | | | | | |
| DdCBE_ | | 99.93% | 0.01% | 0.00% | 99.96% | 99.95% | 0.03% | 0.01% | 0.00% | 99.94% | 0.01% | 0.01% | 0.01% |
| replicate3 | | | | | | | | | | | | | |
| ND5.2- | G | | | | | | | | | | | | |
| DdCBE_ | | 0.00% | 0.01% | 99.80% | 0.00% | 0.00% | 0.01% | 0.01% | 99.97% | 0.00% | 0.01% | 0.01% | 0.00% |
| replicate3 | | | | | | | | | | | | | |
| ND5.2- | N | | | | | | | | | | | | |
| DdCBE_ | | 0.00% | 0.00% | 0.01% | 0.00% | 0.00% | 0.00% | 0.01% | 0.00% | 0.00% | 0.00% | 0.00% | 0.00% |
| replicate3 | | | | | | | | | | | | | |
| ND5.2- | — | | | | | | | | | | | | |
| DdCBE_ | | 0.00% | 0.00% | 0.00% | 0.00% | 0.00% | 0.00% | 0.00% | 0.00% | 0.00% | 0.00% | 0.00% | 0.00% |
| replicate3 | | | | | | | | | | | | | |

Base Percentages at TALE Binding Sites-ND5.2-DdCBE

| Batch | Nucleotide | A | G | C | A | G | G | A | A | T |
|---|---|---|---|---|---|---|---|---|---|---|
| ND5.2- | A | 99.86% | 0.02% | 0.03% | 99.97% | 0.01% | 0.02% | 99.95% | 99.95% | 0.01% |
| DdCBE_ | | | | | | | | | | |
| replicate1 | | | | | | | | | | |
| ND5.2- | T | 0.01% | 0.02% | 0.03% | 0.01% | 0.01% | 0.02% | 0.02% | 0.02% | 99.97% |
| DdCBE_ | | | | | | | | | | |
| replicate1 | | | | | | | | | | |
| ND5.2- | C | 0.00% | 0.01% | 99.94% | 0.00% | 0.00% | 0.00% | 0.01% | 0.01% | 0.02% |
| DdCBE_ | | | | | | | | | | |
| replicate 1 | | | | | | | | | | |
| ND5.2- | G | 0.13% | 99.96% | 0.00% | 0.02% | 99.97% | 99.95% | 0.02% | 0.01% | 0.00% |
| DdCBE_ | | | | | | | | | | |
| replicate1 | | | | | | | | | | |
| ND5.2- | N | 0.00% | 0.00% | 0.00% | 0.00% | 0.00% | 0.00% | 0.00% | 0.00% | 0.00% |
| DdCBE_ | | | | | | | | | | |
| replicate1 | | | | | | | | | | |
| ND5.2- | — | 0.01% | 0.00% | 0.00% | 0.00% | 0.00% | 0.00% | 0.00% | 0.00% | 0.00% |
| DdCBE_ | | | | | | | | | | |
| replicate1 | | | | | | | | | | |
| ND5.2- | A | 99.87% | 0.03% | 0.00% | 100.00% | 0.00% | 0.03% | 100.00% | 99.94% | 0.00% |
| DdCBE_ | | | | | | | | | | |
| replicate2 | | | | | | | | | | |
| ND5.2- | T | 0.00% | 0.00% | 0.00% | 0.00% | 0.03% | 0.03% | 0.00% | 0.00% | 100.00% |
| DdCBE_ | | | | | | | | | | |
| replicate2 | | | | | | | | | | |
| ND5.2- | C | 0.00% | 0.00% | 99.97% | 0.00% | 0.00% | 0.00% | 0.00% | 0.03% | 0.00% |
| DdCBE_ | | | | | | | | | | |
| replicate2 | | | | | | | | | | |
| ND5.2- | G | 0.13% | 99.97% | 0.03% | 0.00% | 99.97% | 99.94% | 0.00% | 0.03% | 0.00% |
| DdCBE_ | | | | | | | | | | |
| replicate2 | | | | | | | | | | |
| ND5.2- | N | 0.00% | 0.00% | 0.00% | 0.00% | 0.00% | 0.00% | 0.00% | 0.00% | 0.00% |
| DdCBE_ | | | | | | | | | | |
| replicate2 | | | | | | | | | | |
| ND5.2- | — | 0.00% | 0.00% | 0.00% | 0.00% | 0.00% | 0.00% | 0.00% | 0.00% | 0.00% |
| DdCBE_ | | | | | | | | | | |
| replicate2 | | | | | | | | | | |
| ND5.2- | A | 99.80% | 0.01% | 0.02% | 99.96% | 0.01% | 0.01% | 99.93% | 99.95% | 0.01% |
| DdCBE_ | | | | | | | | | | |
| replicate3 | | | | | | | | | | |
| ND5.2- | T | 0.01% | 0.02% | 0.02% | 0.01% | 0.03% | 0.04% | 0.03% | 0.02% | 99.98% |
| DdCBE_ | | | | | | | | | | |
| replicate3 | | | | | | | | | | |
| ND5.2- | C | 0.02% | 0.01% | 99.96% | 0.01% | 0.00% | 0.01% | 0.02% | 0.02% | 0.01% |
| DdCBE_ | | | | | | | | | | |
| replicate3 | | | | | | | | | | |
| ND5.2- | G | 0.17% | 99.95% | 0.00% | 0.02% | 99.97% | 99.94% | 0.02% | 0.01% | 0.00% |
| DdCBE_ | | | | | | | | | | |
| replicate3 | | | | | | | | | | |
| ND5.2- | N | 0.00% | 0.00% | 0.00% | 0.00% | 0.00% | 0.00% | 0.00% | 0.00% | 0.00% |
| DdCBE_ | | | | | | | | | | |
| replicate3 | | | | | | | | | | |
| ND5.2- | — | 0.00% | 0.00% | 0.00% | 0.00% | 0.00% | 0.01% | 0.00% | 0.00% | 0.00% |
| DdCBE_ | | | | | | | | | | |
| replicate3 | | | | | | | | | | |

Base Percentages at TALE Binding Sites-ND5.2-DdCBE

| Batch | Nucleotide | A | C | C | T | T | T | C | C | T |
|---|---|---|---|---|---|---|---|---|---|---|
| ND5.2- | A | 99.97% | 0.02% | 0.02% | 0.03% | 0.00% | 0.01% | 0.02% | 0.02% | 0.01% |
| DdCBE_ | | | | | | | | | | |
| replicate1 | | | | | | | | | | |
| ND5.2- | T | 0.01% | 0.02% | 0.01% | 99.81% | 99.98% | 99.97% | 0.02% | 0.01% | 99.97% |
| DdCBE_ | | | | | | | | | | |
| replicate1 | | | | | | | | | | |
| ND5.2- | C | 0.01% | 99.96% | 99.96% | 0.15% | 0.02% | 0.01% | 99.96% | 99.97% | 0.01% |
| DdCBE_ | | | | | | | | | | |
| replicate 1 | | | | | | | | | | |
| ND5.2- | G | 0.01% | 0.00% | 0.00% | 0.01% | 0.00% | 0.00% | 0.00% | 0.00% | 0.00% |
| DdCBE_ | | | | | | | | | | |
| replicate1 | | | | | | | | | | |
| ND5.2- | N | 0.00% | 0.00% | 0.00% | 0.00% | 0.00% | 0.00% | 0.00% | 0.00% | 0.00% |
| DdCBE_ | | | | | | | | | | |
| replicate1 | | | | | | | | | | |
| ND5.2- | — | 0.00% | 0.00% | 0.00% | 0.00% | 0.00% | 0.00% | 0.00% | 0.00% | 0.00% |
| DdCBE_ | | | | | | | | | | |
| replicate1 | | | | | | | | | | |
| ND5.2- | A | 99.97% | 0.00% | 0.06% | 0.03% | 0.00% | 0.00% | 0.00% | 0.03% | 0.03% |
| DdCBE_ | | | | | | | | | | |
| replicate2 | | | | | | | | | | |
| ND5.2- | T | 0.03% | 0.00% | 0.00% | 99.84% | 99.97% | 100.00% | 0.00% | 0.00% | 99.97% |
| DdCBE_ | | | | | | | | | | |
| replicate2 | | | | | | | | | | |
| ND5.2- | C | 0.00% | 100.00% | 99.94% | 0.13% | 0.03% | 0.00% | 99.97% | 99.97% | 0.00% |
| DdCBE_ | | | | | | | | | | |
| replicate2 | | | | | | | | | | |
| ND5.2- | G | 0.00% | 0.00% | 0.00% | 0.00% | 0.00% | 0.00% | 0.03% | 0.00% | 0.00% |
| DdCBE_ | | | | | | | | | | |
| replicate2 | | | | | | | | | | |
| ND5.2- | N | 0.00% | 0.00% | 0.00% | 0.00% | 0.00% | 0.00% | 0.00% | 0.00% | 0.00% |
| DdCBE_ | | | | | | | | | | |
| replicate2 | | | | | | | | | | |
| ND5.2- | — | 0.00% | 0.00% | 0.00% | 0.00% | 0.00% | 0.00% | 0.00% | 0.00% | 0.00% |
| DdCBE_ | | | | | | | | | | |
| replicate2 | | | | | | | | | | |
| ND5.2- | A | 99.97% | 0.02% | 0.02% | 0.02% | 0.00% | 0.01% | 0.02% | 0.02% | 0.01% |
| DdCBE_ | | | | | | | | | | |
| replicate3 | | | | | | | | | | |
| ND5.2- | T | 0.01% | 0.01% | 0.01% | 99.81% | 99.98% | 99.97% | 0.02% | 0.01% | 99.98% |
| DdCBE_ | | | | | | | | | | |
| replicate3 | | | | | | | | | | |
| ND5.2- | C | 0.01% | 99.97% | 99.96% | 0.16% | 0.01% | 0.01% | 99.96% | 99.97% | 0.01% |
| DdCBE_ | | | | | | | | | | |
| replicate3 | | | | | | | | | | |
| ND5.2- | G | 0.00% | 0.00% | 0.00% | 0.01% | 0.00% | 0.00% | 0.00% | 0.00% | 0.00% |
| DdCBE_ | | | | | | | | | | |
| replicate3 | | | | | | | | | | |
| ND5.2- | N | 0.00% | 0.00% | 0.00% | 0.00% | 0.00% | 0.00% | 0.00% | 0.00% | 0.00% |
| DdCBE_ | | | | | | | | | | |
| replicate3 | | | | | | | | | | |
| ND5.2- | — | 0.00% | 0.00% | 0.00% | 0.00% | 0.00% | 0.00% | 0.00% | 0.00% | 0.00% |
| DdCBE_ | | | | | | | | | | |
| replicate3 | | | | | | | | | | |

TABLE 12D
Base Percentages at TALE Binding Sites-ND5.3-DdCBE

| Batch | Nucleotide | A | A | C | T | C | A | G | A |
|---|---|---|---|---|---|---|---|---|---|
| ND5.3- | A | 99.96% | 99.98% | 0.01% | 0.00% | 0.01% | 99.99% | 0.03% | 99.99% |
| DdCBE_ | | | | | | | | | |
| replicate1 | | | | | | | | | |
| ND5.3- | T | 0.01% | 0.00% | 0.01% | 99.98% | 0.05% | 0.00% | 0.00% | 0.01% |
| DdCBE_ | | | | | | | | | |
| replicate 1 | | | | | | | | | |
| ND5.3- | C | 0.01% | 0.00% | 99.99% | 0.01% | 99.95% | 0.00% | 0.00% | 0.00% |
| DdCBE_ | | | | | | | | | |
| replicate1 | | | | | | | | | |
| ND5.3- | G | 0.01% | 0.01% | 0.00% | 0.00% | 0.00% | 0.01% | 99.97% | 0.01% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND5.3- | — | 0.00% | 0.01% | 0.00% | 0.00% | 0.00% | 0.00% | 0.00% | 0.00% |
| DdCBE_ | | | | | | | | | |
| replicate 1 | | | | | | | | | |
| ND5.3- | A | 99.98% | 99.97% | 0.01% | 0.00% | 0.00% | 99.98% | 0.02% | 99.98% |
| DdCBE_ | | | | | | | | | |
| replicate2 | | | | | | | | | |
| ND5.3- | T | 0.01% | 0.00% | 0.01% | 99.98% | 0.03% | 0.00% | 0.00% | 0.01% |
| DdCBE_ | | | | | | | | | |
| replicate2 | | | | | | | | | |
| ND5.3- | C | 0.01% | 0.00% | 99.98% | 0.01% | 99.96% | 0.00% | 0.00% | 0.00% |
| DdCBE_ | | | | | | | | | |
| replicate2 | | | | | | | | | |
| ND5.3- | G | 0.01% | 0.01% | 0.00% | 0.00% | 0.00% | 0.01% | 99.97% | 0.01% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.01% | 0.00% | 0.00% | 0.00% | 0.00% | 0.00% | 0.00% |
| DdCBE_ | | | | | | | | | |
| replicate2 | | | | | | | | | |
| ND5.3- | A | 99.98% | 99.97% | 0.01% | 0.00% | 0.01% | 99.97% | 0.01% | 99.98% |
| DdCBE_ | | | | | | | | | |
| replicate3 | | | | | | | | | |
| ND5.3- | T | 0.00% | 0.00% | 0.01% | 99.98% | 0.04% | 0.00% | 0.01% | 0.01% |
| DdCBE_ | | | | | | | | | |
| replicate3 | | | | | | | | | |
| ND5.3- | C | 0.00% | 0.01% | 99.98% | 0.02% | 99.94% | 0.00% | 0.00% | 0.00% |
| DdCBE_ | | | | | | | | | |
| replicate3 | | | | | | | | | |
| ND5.3- | G | 0.01% | 0.01% | 0.00% | 0.00% | 0.00% | 0.03% | 99.98% | 0.01% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.01% | 0.00% | 0.00% | 0.00% | 0.00% | 0.00% | 0.00% |
| DdCBE_ | | | | | | | | | |
| replicate3 | | | | | | | | | |
| Batch | Nucleotide | T | T | C | T | T | C | A | A |
| ND5.3- | A | 0.02% | 0.01% | 0.00% | 0.01% | 0.01% | 0.01% | 99.97% | 99.97% |
| DdCBE_ | | | | | | | | | |
| replicate 1 | | | | | | | | | |
| ND5.3- | T | 99.94% | 99.97% | 0.00% | 99.97% | 99.97% | 0.01% | 0.01% | 0.01% |
| DdCBE_ | | | | | | | | | |
| replicate 1 | | | | | | | | | |
| ND5.3- | C | 0.02% | 0.01% | 99.99% | 0.02% | 0.02% | 99.98% | 0.00% | 0.00% |
| DdCBE_ | | | | | | | | | |
| replicate 1 | | | | | | | | | |
| ND5.3- | G | 0.02% | 0.00% | 0.00% | 0.01% | 0.00% | 0.00% | 0.01% | 0.02% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | A | 0.01% | 0.01% | 0.01% | 0.00% | 0.01% | 0.01% | 99.96% | 99.96% |
| DdCBE_ | | | | | | | | | |
| replicate2 | | | | | | | | | |
| ND5.3- | T | 99.95% | 99.99% | 0.02% | 99.98% | 99.97% | 0.02% | 0.01% | 0.01% |
| DdCBE_ | | | | | | | | | |
| replicate2 | | | | | | | | | |
| ND5.3- | C | 0.02% | 0.00% | 99.98% | 0.01% | 0.01% | 99.96% | 0.00% | 0.01% |
| DdCBE_ | | | | | | | | | |
| replicate2 | | | | | | | | | |
| ND5.3- | G | 0.02% | 0.00% | 0.00% | 0.00% | 0.01% | 0.00% | 0.02% | 0.02% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | A | 0.01% | 0.01% | 0.00% | 0.00% | 0.01% | 0.01% | 99.96% | 99.96% |
| DdCBE_ | | | | | | | | | |
| replicate3 | | | | | | | | | |
| ND5.3- | T | 99.94% | 99.97% | 0.01% | 99.99% | 99.98% | 0.01% | 0.01% | 0.01% |
| DdCBE_ | | | | | | | | | |
| replicate3 | | | | | | | | | |
| ND5.3- | C | 0.02% | 0.01% | 99.99% | 0.01% | 0.01% | 99.98% | 0.00% | 0.01% |
| DdCBE_ | | | | | | | | | |
| replicate3 | | | | | | | | | |
| ND5.3- | G | 0.03% | 0.00% | 0.00% | 0.00% | 0.00% | 0.00% | 0.03% | 0.03% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | C | T | A | A | T | T | A | C |
| ND5.3- | A | 0.00% | 0.00% | 99.98% | 99.98% | 0.00% | 0.00% | 99.99% | 0.00% |
| DdCBE_ | | | | | | | | | |
| replicate 1 | | | | | | | | | |
| ND5.3- | T | 5.40% | 99.99% | 0.00% | 0.00% | 99.99% | 99.98% | 0.01% | 0.01% |
| DdCBE_ | | | | | | | | | |
| replicate 1 | | | | | | | | | |
| ND5.3- | C | 94.60% | 0.01% | 0.00% | 0.01% | 0.00% | 0.01% | 0.00% | 99.99% |
| DdCBE_ | | | | | | | | | |
| replicate1 | | | | | | | | | |
| ND5.3- | G | 0.00% | 0.00% | 0.01% | 0.00% | 0.00% | 0.00% | 0.01% | 0.00% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | — | 0.00% | 0.00% | 0.00% | 0.01% | 0.00% | 0.01% | 0.00% | 0.00% |
| DdCBE_ | | | | | | | | | |
| replicate 1 | | | | | | | | | |
| ND5.3- | A | 0.00% | 0.00% | 99.98% | 99.96% | 0.00% | 0.01% | 99.98% | 0.01% |
| DdCBE_ | | | | | | | | | |
| replicate2 | | | | | | | | | |
| ND5.3- | T | 5.00% | 99.97% | 0.00% | 0.01% | 99.99% | 99.97% | 0.01% | 0.01% |
| DdCBE_ | | | | | | | | | |
| replicate2 | | | | | | | | | |
| ND5.3- | C | 94.99% | 0.01% | 0.00% | 0.01% | 0.00% | 0.02% | 0.00% | 99.98% |
| DdCBE_ | | | | | | | | | |
| replicate2 | | | | | | | | | |
| ND5.3- | G | 0.00% | 0.00% | 0.01% | 0.01% | 0.00% | 0.00% | 0.01% | 0.00% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.01% | 0.00% | 0.01% | 0.00% | 0.00% | 0.00% | 0.00% |
| DdCBE_ | | | | | | | | | |
| replicate2 | | | | | | | | | |
| ND5.3- | A | 0.01% | 0.01% | 99.98% | 99.96% | 0.00% | 0.01% | 99.99% | 0.00% |
| DdCBE_ | | | | | | | | | |
| replicate3 | | | | | | | | | |
| ND5.3- | T | 5.27% | 99.98% | 0.00% | 0.00% | 99.99% | 99.98% | 0.00% | 0.01% |
| DdCBE_ | | | | | | | | | |
| replicate3 | | | | | | | | | |
| ND5.3- | C | 94.72% | 0.01% | 0.01% | 0.02% | 0.01% | 0.01% | 0.00% | 99.99% |
| DdCBE_ | | | | | | | | | |
| replicate3 | | | | | | | | | |
| ND5.3- | G | 0.00% | 0.00% | 0.01% | 0.01% | 0.00% | 0.00% | 0.01% | 0.00% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.00% | 0.00% | 0.01% | 0.00% | 0.00% |
| DdCBE_ | | | | | | | | | |
| replicate3 | | | | | | | | | |
| Batch | Nucleotide | A | C | C | G | C | T | A | A |
| ND5.3- | A | 99.99% | 0.00% | 0.02% | 0.02% | 0.00% | 0.00% | 99.98% | 99.96% |
| DdCBE_ | | | | | | | | | |
| replicate 1 | | | | | | | | | |
| ND5.3- | T | 0.01% | 0.01% | 0.02% | 0.01% | 0.01% | 99.99% | 0.00% | 0.01% |
| DdCBE_ | | | | | | | | | |
| replicate1 | | | | | | | | | |
| ND5.3- | C | 0.00% | 99.99% | 99.96% | 0.01% | 99.98% | 0.01% | 0.01% | 0.01% |
| DdCBE_ | | | | | | | | | |
| replicate 1 | | | | | | | | | |
| ND5.3- | G | 0.00% | 0.00% | 0.01% | 99.97% | 0.01% | 0.00% | 0.01% | 0.01% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND5.3- | — | 0.00% | 0.00% | 0.00% | 0.00% | 0.00% | 0.00% | 0.00% | 0.01% |
| DdCBE_ | | | | | | | | | |
| replicate 1 | | | | | | | | | |
| ND5.3- | A | 99.97% | 0.01% | 0.01% | 0.02% | 0.01% | 0.01% | 99.98% | 99.93% |
| DdCBE_ | | | | | | | | | |
| replicate2 | | | | | | | | | |
| ND5.3- | T | 0.01% | 0.00% | 0.02% | 0.01% | 0.01% | 99.97% | 0.00% | 0.02% |
| DdCBE_ | | | | | | | | | |
| replicate2 | | | | | | | | | |
| ND5.3- | C | 0.01% | 99.99% | 99.96% | 0.01% | 99.98% | 0.02% | 0.00% | 0.02% |
| DdCBE_ | | | | | | | | | |
| replicate2 | | | | | | | | | |
| ND5.3- | G | 0.00% | 0.00% | 0.01% | 99.96% | 0.00% | 0.00% | 0.01% | 0.01% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.01% | 0.00% | 0.00% | 0.00% | 0.00% | 0.01% | 0.00% | 0.01% |
| DdCBE_ | | | | | | | | | |
| replicate2 | | | | | | | | | |
| ND5.3- | A | 99.96% | 0.01% | 0.01% | 0.02% | 0.01% | 0.01% | 99.98% | 99.96% |
| DdCBE_ | | | | | | | | | |
| replicate3 | | | | | | | | | |
| ND5.3- | T | 0.01% | 0.01% | 0.01% | 0.01% | 0.00% | 99.99% | 0.00% | 0.01% |
| DdCBE_ | | | | | | | | | |
| replicate3 | | | | | | | | | |
| ND5.3- | C | 0.00% | 99.98% | 99.98% | 0.01% | 99.99% | 0.01% | 0.00% | 0.01% |
| DdCBE_ | | | | | | | | | |
| replicate3 | | | | | | | | | |
| ND5.3- | G | 0.02% | 0.00% | 0.01% | 99.97% | 0.00% | 0.00% | 0.01% | 0.01% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.00% | 0.00% | 0.00% | 0.00% | 0.01% |
| DdCBE_ | | | | | | | | | |
| replicate3 | | | | | | | | | |
| Batch | Nucleotide | C | C | C | A | A | A | C | A |
| ND5.3- | A | 0.01% | 0.01% | 0.01% | 99.97% | 99.98% | 99.98% | 0.02% | 99.96% |
| DdCBE_ | | | | | | | | | |
| replicate1 | | | | | | | | | |
| ND5.3- | T | 0.01% | 0.01% | 0.01% | 0.00% | 0.00% | 0.01% | 0.01% | 0.01% |
| DdCBE_ | | | | | | | | | |
| replicate 1 | | | | | | | | | |
| ND5.3- | C | 99.98% | 99.97% | 99.97% | 0.00% | 0.00% | 0.00% | 99.97% | 0.02% |
| DdCBE_ | | | | | | | | | |
| replicate1 | | | | | | | | | |
| ND5.3- | G | 0.00% | 0.00% | 0.00% | 0.02% | 0.02% | 0.01% | 0.00% | 0.01% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND5.3- | A | 0.00% | 0.01% | 0.00% | 99.98% | 99.98% | 99.98% | 0.02% | 99.96% |
| DdCBE_ | | | | | | | | | |
| replicate2 | | | | | | | | | |
| ND5.3- | T | 0.01% | 0.01% | 0.01% | 0.00% | 0.01% | 0.00% | 0.01% | 0.00% |
| DdCBE_ | | | | | | | | | |
| replicate2 | | | | | | | | | |
| ND5.3- | C | 99.99% | 99.98% | 99.98% | 0.00% | 0.01% | 0.01% | 99.97% | 0.02% |
| DdCBE_ | | | | | | | | | |
| replicate2 | | | | | | | | | |
| ND5.3- | G | 0.00% | 0.00% | 0.00% | 0.02% | 0.01% | 0.01% | 0.00% | 0.02% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | A | 0.01% | 0.01% | 0.01% | 99.97% | 99.97% | 99.96% | 0.03% | 99.96% |
| DdCBE_ | | | | | | | | | |
| replicate3 | | | | | | | | | |
| ND5.3- | T | 0.01% | 0.01% | 0.01% | 0.00% | 0.01% | 0.01% | 0.01% | 0.00% |
| DdCBE_ | | | | | | | | | |
| replicate3 | | | | | | | | | |
| ND5.3- | C | 99.98% | 99.98% | 99.97% | 0.01% | 0.00% | 0.01% | 99.96% | 0.02% |
| DdCBE_ | | | | | | | | | |
| replicate3 | | | | | | | | | |
| ND5.3- | G | 0.00% | 0.00% | 0.00% | 0.01% | 0.02% | 0.01% | 0.01% | 0.01% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.00% | 0.00% | 0.01% | 0.00% | 0.00% |
| DdCBE_ | | | | | | | | | |
| replicate3 | | | | | | | | | |
| Batch | Nucleotide | A | T | A | T | C | T | A | C |
| ND5.3- | A | 99.99% | 0.01% | 99.99% | 0.01% | 0.01% | 0.00% | 99.97% | 0.00% |
| DdCBE_ | | | | | | | | | |
| replicate 1 | | | | | | | | | |
| ND5.3- | T | 0.00% | 99.98% | 0.01% | 99.99% | 0.01% | 99.99% | 0.02% | 22.67% |
| DdCBE_ | | | | | | | | | |
| replicate 1 | | | | | | | | | |
| ND5.3- | C | 0.00% | 0.01% | 0.00% | 0.00% | 99.98% | 0.01% | 0.01% | 77.32% |
| DdCBE_ | | | | | | | | | |
| replicate 1 | | | | | | | | | |
| ND5.3- | G | 0.01% | 0.00% | 0.01% | 0.00% | 0.00% | 0.00% | 0.01% | 0.00% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | A | 99.99% | 0.01% | 99.98% | 0.00% | 0.00% | 0.01% | 99.98% | 0.00% |
| DdCBE_ | | | | | | | | | |
| replicate2 | | | | | | | | | |
| ND5.3- | T | 0.00% | 99.98% | 0.01% | 99.99% | 0.02% | 99.97% | 0.01% | 21.07% |
| DdCBE_ | | | | | | | | | |
| replicate2 | | | | | | | | | |
| ND5.3- | C | 0.00% | 0.02% | 0.00% | 0.01% | 99.98% | 0.02% | 0.00% | 78.92% |
| DdCBE_ | | | | | | | | | |
| replicate2 | | | | | | | | | |
| ND5.3- | G | 0.01% | 0.00% | 0.01% | 0.00% | 0.00% | 0.00% | 0.01% | 0.00% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | A | 99.98% | 0.00% | 99.98% | 0.01% | 0.01% | 0.01% | 99.99% | 0.01% |
| DdCBE_ | | | | | | | | | |
| replicate3 | | | | | | | | | |
| ND5.3- | T | 0.01% | 99.99% | 0.01% | 99.99% | 0.01% | 99.99% | 0.01% | 16.15% |
| DdCBE_ | | | | | | | | | |
| replicate3 | | | | | | | | | |
| ND5.3- | C | 0.00% | 0.01% | 0.00% | 0.01% | 99.98% | 0.01% | 0.00% | 83.84% |
| DdCBE_ | | | | | | | | | |
| replicate3 | | | | | | | | | |
| ND5.3- | G | 0.00% | 0.00% | 0.01% | 0.00% | 0.00% | 0.00% | 0.00% | 0.00% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | C | A | T | A | C | T | A | A |
| ND5.3- | A | 0.01% | 99.98% | 0.00% | 99.97% | 0.00% | 0.01% | 99.99% | 99.96% |
| DdCBE_ | | | | | | | | | |
| replicate 1 | | | | | | | | | |
| ND5.3- | T | 0.01% | 0.01% | 99.98% | 0.01% | 0.01% | 99.97% | 0.00% | 0.01% |
| DdCBE_ | | | | | | | | | |
| replicate 1 | | | | | | | | | |
| ND5.3- | C | 99.98% | 0.00% | 0.01% | 0.01% | 99.99% | 0.01% | 0.00% | 0.02% |
| DdCBE_ | | | | | | | | | |
| replicate1 | | | | | | | | | |
| ND5.3- | G | 0.00% | 0.01% | 0.00% | 0.01% | 0.00% | 0.00% | 0.01% | 0.01% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND5.3- | A | 0.01% | 99.97% | 0.00% | 99.97% | 0.01% | 0.00% | 99.99% | 99.96% |
| DdCBE_ | | | | | | | | | |
| replicate2 | | | | | | | | | |
| ND5.3- | T | 0.01% | 0.02% | 99.98% | 0.01% | 0.00% | 99.98% | 0.00% | 0.02% |
| DdCBE_ | | | | | | | | | |
| replicate2 | | | | | | | | | |
| ND5.3- | C | 99.98% | 0.00% | 0.01% | 0.01% | 99.99% | 0.01% | 0.00% | 0.01% |
| DdCBE_ | | | | | | | | | |
| replicate2 | | | | | | | | | |
| ND5.3- | G | 0.00% | 0.00% | 0.00% | 0.01% | 0.00% | 0.00% | 0.01% | 0.01% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | A | 0.01% | 99.98% | 0.01% | 99.98% | 0.01% | 0.01% | 99.99% | 99.98% |
| DdCBE_ | | | | | | | | | |
| replicate3 | | | | | | | | | |
| ND5.3- | T | 0.01% | 0.01% | 99.98% | 0.01% | 0.01% | 99.97% | 0.00% | 0.01% |
| DdCBE_ | | | | | | | | | |
| replicate3 | | | | | | | | | |
| ND5.3- | C | 99.97% | 0.00% | 0.01% | 0.00% | 99.99% | 0.01% | 0.00% | 0.01% |
| DdCBE_ | | | | | | | | | |
| replicate3 | | | | | | | | | |
| ND5.3- | G | 0.00% | 0.01% | 0.00% | 0.01% | 0.00% | 0.00% | 0.01% | 0.00% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.01% | 0.00% | 0.00% | 0.00% | 0.00% | 0.01% | 0.00% | 0.01% |
| DdCBE_ | | | | | | | | | |
| replicate3 | | | | | | | | | |
| Batch | Nucleotide | C | A | A | C | C | T | A | T |
| ND5.3- | A | 0.01% | 99.98% | 99.96% | 0.01% | 0.00% | 0.00% | 99.97% | 0.00% |
| DdCBE_ | | | | | | | | | |
| replicate 1 | | | | | | | | | |
| ND5.3- | T | 0.01% | 0.00% | 0.01% | 0.01% | 0.01% | 99.98% | 0.02% | 99.99% |
| DdCBE_ | | | | | | | | | |
| replicate1 | | | | | | | | | |
| ND5.3- | C | 99.97% | 0.00% | 0.01% | 99.98% | 99.98% | 0.01% | 0.01% | 0.01% |
| DdCBE_ | | | | | | | | | |
| replicate 1 | | | | | | | | | |
| ND5.3- | G | 0.01% | 0.01% | 0.01% | 0.00% | 0.00% | 0.00% | 0.01% | 0.00% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND5.3- | — | 0.00% | 0.00% | 0.01% | 0.00% | 0.00% | 0.00% | 0.00% | 0.00% |
| DdCBE_ | | | | | | | | | |
| replicate 1 | | | | | | | | | |
| ND5.3- | A | 0.01% | 99.99% | 99.98% | 0.00% | 0.02% | 0.01% | 99.97% | 0.00% |
| DdCBE_ | | | | | | | | | |
| replicate2 | | | | | | | | | |
| ND5.3- | T | 0.01% | 0.00% | 0.01% | 0.01% | 0.01% | 99.98% | 0.01% | 99.98% |
| DdCBE_ | | | | | | | | | |
| replicate2 | | | | | | | | | |
| ND5.3- | C | 99.98% | 0.00% | 0.01% | 99.99% | 99.97% | 0.01% | 0.00% | 0.02% |
| DdCBE_ | | | | | | | | | |
| replicate2 | | | | | | | | | |
| ND5.3- | G | 0.00% | 0.01% | 0.01% | 0.00% | 0.00% | 0.00% | 0.01% | 0.00% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | A | 0.01% | 99.98% | 99.97% | 0.01% | 0.01% | 0.01% | 99.96% | 0.00% |
| DdCBE_ | | | | | | | | | |
| replicate3 | | | | | | | | | |
| ND5.3- | T | 0.01% | 0.00% | 0.01% | 0.01% | 0.01% | 99.98% | 0.02% | 99.98% |
| DdCBE_ | | | | | | | | | |
| replicate3 | | | | | | | | | |
| ND5.3- | C | 99.99% | 0.01% | 0.01% | 99.99% | 99.99% | 0.01% | 0.01% | 0.02% |
| DdCBE_ | | | | | | | | | |
| replicate3 | | | | | | | | | |
| ND5.3- | G | 0.00% | 0.01% | 0.01% | 0.00% | 0.00% | 0.00% | 0.01% | 0.00% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | T | T | A | A | T | C | A | G |
| ND5.3- | A | 0.00% | 0.00% | 99.97% | 99.95% | 0.01% | 0.02% | 99.97% | 0.09% |
| DdCBE_ | | | | | | | | | |
| replicate1 | | | | | | | | | |
| ND5.3- | T | 100.00% | 99.98% | 0.02% | 0.02% | 99.98% | 0.01% | 0.01% | 0.01% |
| DdCBE_ | | | | | | | | | |
| replicate 1 | | | | | | | | | |
| ND5.3- | C | 0.00% | 0.01% | 0.01% | 0.01% | 0.01% | 99.97% | 0.00% | 0.00% |
| DdCBE_ | | | | | | | | | |
| replicate1 | | | | | | | | | |
| ND5.3- | G | 0.00% | 0.00% | 0.01% | 0.01% | 0.00% | 0.00% | 0.02% | 99.90% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND5.3- | A | 0.00% | 0.01% | 99.96% | 99.95% | 0.01% | 0.02% | 99.99% | 0.09% |
| DdCBE_ | | | | | | | | | |
| replicate2 | | | | | | | | | |
| ND5.3- | T | 99.98% | 99.98% | 0.02% | 0.03% | 99.98% | 0.01% | 0.00% | 0.00% |
| DdCBE_ | | | | | | | | | |
| replicate2 | | | | | | | | | |
| ND5.3- | C | 0.02% | 0.02% | 0.00% | 0.01% | 0.01% | 99.96% | 0.00% | 0.00% |
| DdCBE_ | | | | | | | | | |
| replicate2 | | | | | | | | | |
| ND5.3- | G | 0.00% | 0.00% | 0.01% | 0.01% | 0.00% | 0.00% | 0.00% | 99.91% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | A | 0.00% | 0.01% | 99.96% | 99.94% | 0.01% | 0.02% | 99.97% | 0.07% |
| DdCBE_ | | | | | | | | | |
| replicate3 | | | | | | | | | |
| ND5.3- | T | 99.99% | 99.98% | 0.03% | 0.03% | 99.98% | 0.01% | 0.01% | 0.00% |
| DdCBE_ | | | | | | | | | |
| replicate3 | | | | | | | | | |
| ND5.3- | C | 0.01% | 0.01% | 0.00% | 0.02% | 0.01% | 99.97% | 0.01% | 0.00% |
| DdCBE_ | | | | | | | | | |
| replicate3 | | | | | | | | | |
| ND5.3- | G | 0.00% | 0.00% | 0.01% | 0.01% | 0.00% | 0.00% | 0.01% | 99.93% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | T | C | A | T | C | T | T | C |
| ND5.3- | A | 0.02% | 0.01% | 99.98% | 0.00% | 0.00% | 0.01% | 0.00% | 0.01% |
| DdCBE_ | | | | | | | | | |
| replicate 1 | | | | | | | | | |
| ND5.3- | T | 99.98% | 0.02% | 0.00% | 99.98% | 0.10% | 99.97% | 99.98% | 28.54% |
| DdCBE_ | | | | | | | | | |
| replicate 1 | | | | | | | | | |
| ND5.3- | C | 0.00% | 99.97% | 0.01% | 0.01% | 99.90% | 0.02% | 0.02% | 71.45% |
| DdCBE_ | | | | | | | | | |
| replicate 1 | | | | | | | | | |
| ND5.3- | G | 0.00% | 0.00% | 0.01% | 0.00% | 0.00% | 0.00% | 0.00% | 0.00% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | A | 0.02% | 0.01% | 99.96% | 0.01% | 0.00% | 0.01% | 0.01% | 0.00% |
| DdCBE_ | | | | | | | | | |
| replicate2 | | | | | | | | | |
| ND5.3- | T | 99.98% | 0.02% | 0.01% | 99.98% | 0.11% | 99.98% | 99.97% | 26.39% |
| DdCBE_ | | | | | | | | | |
| replicate2 | | | | | | | | | |
| ND5.3- | C | 0.01% | 99.97% | 0.01% | 0.01% | 99.89% | 0.01% | 0.02% | 73.61% |
| DdCBE_ | | | | | | | | | |
| replicate2 | | | | | | | | | |
| ND5.3- | G | 0.00% | 0.00% | 0.01% | 0.00% | 0.00% | 0.00% | 0.01% | 0.00% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | A | 0.01% | 0.01% | 99.98% | 0.00% | 0.00% | 0.01% | 0.00% | 0.00% |
| DdCBE_ | | | | | | | | | |
| replicate3 | | | | | | | | | |
| ND5.3- | T | 99.98% | 0.03% | 0.00% | 99.99% | 0.11% | 99.97% | 99.98% | 25.96% |
| DdCBE_ | | | | | | | | | |
| replicate3 | | | | | | | | | |
| ND5.3- | C | 0.01% | 99.96% | 0.01% | 0.01% | 99.89% | 0.01% | 0.01% | 74.04% |
| DdCBE_ | | | | | | | | | |
| replicate3 | | | | | | | | | |
| ND5.3- | G | 0.00% | 0.01% | 0.01% | 0.00% | 0.00% | 0.00% | 0.01% | 0.00% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | T | C | T | T | A | G | T | T |
| ND5.3- | A | 0.01% | 0.00% | 0.01% | 0.01% | 99.99% | 0.01% | 0.02% | 0.01% |
| DdCBE_ | | | | | | | | | |
| replicate 1 | | | | | | | | | |
| ND5.3- | T | 99.98% | 0.01% | 99.98% | 99.97% | 0.00% | 0.01% | 99.97% | 99.97% |
| DdCBE_ | | | | | | | | | |
| replicate 1 | | | | | | | | | |
| ND5.3- | C | 0.01% | 99.98% | 0.01% | 0.02% | 0.00% | 0.00% | 0.01% | 0.01% |
| DdCBE_ | | | | | | | | | |
| replicate1 | | | | | | | | | |
| ND5.3- | G | 0.00% | 0.00% | 0.00% | 0.00% | 0.01% | 99.97% | 0.00% | 0.00% |
| DdCBE_ | | | | | | | | | |
| replicate 1 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND5.3- | — | 0.00% | 0.00% | 0.00% | 0.01% | 0.00% | 0.00% | 0.00% | 0.01% |
| DdCBE_ | | | | | | | | | |
| replicate 1 | | | | | | | | | |
| ND5.3- | A | 0.00% | 0.00% | 0.00% | 0.01% | 99.98% | 0.01% | 0.02% | 0.01% |
| DdCBE_ | | | | | | | | | |
| replicate2 | | | | | | | | | |
| ND5.3- | T | 99.98% | 0.01% | 99.98% | 99.97% | 0.00% | 0.02% | 99.97% | 99.97% |
| DdCBE_ | | | | | | | | | |
| replicate2 | | | | | | | | | |
| ND5.3- | C | 0.02% | 99.99% | 0.01% | 0.02% | 0.01% | 0.00% | 0.01% | 0.01% |
| DdCBE_ | | | | | | | | | |
| replicate2 | | | | | | | | | |
| ND5.3- | G | 0.00% | 0.00% | 0.00% | 0.00% | 0.02% | 99.97% | 0.00% | 0.00% |
| DdCBE_ | | | | | | | | | |
| replicate2 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND5.3- | — | 0.00% | 0.00% | 0.00% | 0.01% | 0.00% | 0.00% | 0.00% | 0.01% |
| DdCBE_ | | | | | | | | | |
| replicate2 | | | | | | | | | |
| ND5.3- | A | 0.00% | 0.00% | 0.01% | 0.00% | 99.99% | 0.01% | 0.01% | 0.01% |
| DdCBE_ | | | | | | | | | |
| replicate3 | | | | | | | | | |
| ND5.3- | T | 99.99% | 0.00% | 99.97% | 99.97% | 0.00% | 0.01% | 99.98% | 99.98% |
| DdCBE_ | | | | | | | | | |
| replicate3 | | | | | | | | | |
| ND5.3- | C | 0.01% | 99.99% | 0.01% | 0.01% | 0.00% | 0.00% | 0.01% | 0.00% |
| DdCBE_ | | | | | | | | | |
| replicate3 | | | | | | | | | |
| ND5.3- | G | 0.00% | 0.00% | 0.00% | 0.00% | 0.01% | 99.97% | 0.00% | 0.00% |
| DdCBE_ | | | | | | | | | |
| replicate3 | | | | | | | | | |
| ND5.3- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND5.3- | — | 0.00% | 0.00% | 0.00% | 0.01% | 0.00% | 0.00% | 0.00% | 0.01% |
| DdCBE_ | | | | | | | | | |
| replicate3 | | | | | | | | | |

| Batch | Nucleotide | T |
|---|---|---|
| ND5.3- | A | 0.01% |
| DdCBE_ | | |
| replicate 1 | | |
| ND5.3- | T | 99.98% |
| DdCBE_ | | |
| replicate1 | | |
| ND5.3- | C | 0.01% |
| DdCBE_ | | |
| replicate 1 | | |
| ND5.3- | G | 0.00% |
| DdCBE_ | | |
| replicate 1 | | |
| ND5.3- | N | 0.00% |
| DdCBE_ | | |
| replicate 1 | | |
| ND5.3- | — | 0.00% |
| DdCBE_ | | |
| replicate 1 | | |
| ND5.3- | A | 0.01% |
| DdCBE_ | | |
| replicate2 | | |
| ND5.3- | T | 99.98% |
| DdCBE_ | | |
| replicate2 | | |
| ND5.3- | C | 0.01% |
| DdCBE_ | | |
| replicate2 | | |
| ND5.3- | G | 0.00% |
| DdCBE_ | | |
| replicate2 | | |
| ND5.3- | N | 0.00% |
| DdCBE_ | | |
| replicate2 | | |
| ND5.3- | — | 0.00% |
| DdCBE_ | | |
| replicate2 | | |
| ND5.3- | A | 0.01% |
| DdCBE_ | | |
| replicate3 | | |
| ND5.3- | T | 99.97% |
| DdCBE_ | | |
| replicate3 | | |
| ND5.3- | C | 0.01% |
| DdCBE_ | | |
| replicate3 | | |
| ND5.3- | G | 0.00% |
| DdCBE_ | | |
| replicate3 | | |
| ND5.3- | N | 0.00% |
| DdCBE_ | | |
| replicate3 | | |
| ND5.3- | — | 0.00% |
| DdCBE_ | | |
| replicate3 | | |

TABLE 12E
Base Percentages at TALE Binding Sites-ND4-DdCBE

| Batch | Nucleotide | C | C | T | A | C | T | G | G |
|---|---|---|---|---|---|---|---|---|---|
| ND4- | A | 0.00% | 0.02% | 0.01% | 99.98% | 0.00% | 0.01% | 0.25% | 0.01% |
| DdCBE_ | | | | | | | | | |
| replicate 1 | | | | | | | | | |
| ND4- | T | 0.01% | 0.01% | 99.98% | 0.00% | 0.01% | 99.98% | 0.01% | 0.01% |
| DdCBE_ | | | | | | | | | |
| replicate1 | | | | | | | | | |
| ND4- | C | 99.99% | 99.97% | 0.01% | 0.00% | 99.99% | 0.01% | 0.00% | 0.04% |
| DdCBE_ | | | | | | | | | |
| replicate 1 | | | | | | | | | |
| ND4- | G | 0.00% | 0.00% | 0.01% | 0.01% | 0.00% | 0.00% | 99.74% | 99.94% |
| DdCBE_ | | | | | | | | | |
| replicate1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | A | 0.01% | 0.03% | 0.01% | 99.99% | 0.00% | 0.01% | 0.25% | 0.02% |
| DdCBE_ | | | | | | | | | |
| replicate2 | | | | | | | | | |
| ND4- | T | 0.00% | 0.01% | 99.96% | 0.00% | 0.00% | 99.97% | 0.03% | 0.01% |
| DdCBE_ | | | | | | | | | |
| replicate2 | | | | | | | | | |
| ND4 | C | 99.99% | 99.96% | 0.02% | 0.00% | 100.00% | 0.02% | 0.00% | 0.02% |
| DdCBE_ | | | | | | | | | |
| replicate2 | | | | | | | | | |
| ND4 | G | 0.00% | 0.00% | 0.01% | 0.01% | 0.00% | 0.00% | 99.72% | 99.94% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4 | A | 0.00% | 0.02% | 0.00% | 99.98% | 0.00% | 0.01% | 0.20% | 0.01% |
| DdCBE_ | | | | | | | | | |
| replicate3 | | | | | | | | | |
| ND4 | T | 0.00% | 0.01% | 99.97% | 0.01% | 0.01% | 99.98% | 0.02% | 0.02% |
| DdCBE_ | | | | | | | | | |
| replicate3 | | | | | | | | | |
| ND4- | C | 99.99% | 99.96% | 0.02% | 0.01% | 99.99% | 0.01% | 0.00% | 0.03% |
| DdCBE_ | | | | | | | | | |
| replicate3 | | | | | | | | | |
| ND4- | G | 0.00% | 0.00% | 0.01% | 0.00% | 0.00% | 0.00% | 99.77% | 99.94% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | A | G | T | A | A | C | C | A |
| ND4- | A | 99.98% | 0.01% | 0.01% | 99.98% | 99.97% | 0.01% | 0.01% | 99.97% |
| DdCBE_ | | | | | | | | | |
| replicate 1 | | | | | | | | | |
| ND4- | T | 0.00% | 0.01% | 99.91% | 0.00% | 0.02% | 0.01% | 0.01% | 0.01% |
| DdCBE_ | | | | | | | | | |
| replicate1 | | | | | | | | | |
| ND4- | C | 0.01% | 0.00% | 0.09% | 0.01% | 0.00% | 99.98% | 99.98% | 0.01% |
| DdCBE_ | | | | | | | | | |
| replicate1 | | | | | | | | | |
| ND4- | G | 0.01% | 99.98% | 0.00% | 0.01% | 0.01% | 0.00% | 0.00% | 0.01% |
| DdCBE_ | | | | | | | | | |
| replicate1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | — | 0.00% | 0.00% | 0.00% | 0.00% | 0.01% | 0.00% | 0.00% | 0.00% |
| DdCBE_ | | | | | | | | | |
| replicate1 | | | | | | | | | |
| ND4- | A | 99.98% | 0.01% | 0.01% | 99.98% | 99.98% | 0.00% | 0.01% | 99.98% |
| DdCBE_ | | | | | | | | | |
| replicate2 | | | | | | | | | |
| ND4- | T | 0.01% | 0.01% | 99.92% | 0.01% | 0.01% | 0.01% | 0.01% | 0.01% |
| DdCBE_ | | | | | | | | | |
| replicate2 | | | | | | | | | |
| ND4- | C | 0.01% | 0.00% | 0.07% | 0.00% | 0.01% | 99.99% | 99.97% | 0.00% |
| DdCBE_ | | | | | | | | | |
| replicate2 | | | | | | | | | |
| ND4- | G | 0.01% | 99.99% | 0.00% | 0.01% | 0.00% | 0.00% | 0.00% | 0.01% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | A | 99.98% | 0.01% | 0.01% | 99.98% | 99.97% | 0.00% | 0.01% | 99.98% |
| DdCBE_ | | | | | | | | | |
| replicate3 | | | | | | | | | |
| ND4- | T | 0.01% | 0.01% | 99.93% | 0.00% | 0.01% | 0.01% | 0.02% | 0.01% |
| DdCBE_ | | | | | | | | | |
| replicate3 | | | | | | | | | |
| ND4- | C | 0.01% | 0.00% | 0.06% | 0.01% | 0.01% | 99.99% | 99.96% | 0.01% |
| DdCBE_ | | | | | | | | | |
| replicate3 | | | | | | | | | |
| ND4- | G | 0.01% | 99.98% | 0.00% | 0.01% | 0.01% | 0.00% | 0.01% | 0.01% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | — | 0.00% | 0.00% | 0.00% | 0.00% | 0.01% | 0.00% | 0.01% | 0.00% |
| DdCBE_ | | | | | | | | | |
| replicate3 | | | | | | | | | |
| Batch | Nucleotide | T | A | T | C | A | C | T | C |
| ND4- | A | 0.00% | 99.97% | 0.01% | 0.01% | 99.97% | 0.01% | 0.01% | 0.01% |
| DdCBE_ | | | | | | | | | |
| replicate 1 | | | | | | | | | |
| ND4- | T | 99.99% | 0.02% | 99.98% | 0.02% | 0.01% | 0.01% | 99.98% | 0.01% |
| DdCBE_ | | | | | | | | | |
| replicate1 | | | | | | | | | |
| ND4- | C | 0.01% | 0.00% | 0.01% | 99.97% | 0.01% | 99.98% | 0.01% | 99.98% |
| DdCBE_ | | | | | | | | | |
| replicate 1 | | | | | | | | | |
| ND4- | G | 0.00% | 0.01% | 0.00% | 0.00% | 0.01% | 0.00% | 0.00% | 0.00% |
| DdCBE_ | | | | | | | | | |
| replicate 1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND4- | | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND4- | A | 0.01% | 99.98% | 0.00% | 0.00% | 99.95% | 0.00% | 0.01% | 0.00% |
| DdCBE_ | | | | | | | | | |
| replicate2 | | | | | | | | | |
| ND4- | T | 99.98% | 0.00% | 99.99% | 0.02% | 0.01% | 0.01% | 99.98% | 0.01% |
| DdCBE_ | | | | | | | | | |
| replicate2 | | | | | | | | | |
| ND4- | C | 0.01% | 0.01% | 0.01% | 99.98% | 0.03% | 99.99% | 0.01% | 99.99% |
| DdCBE_ | | | | | | | | | |
| replicate2 | | | | | | | | | |
| ND4- | G | 0.00% | 0.01% | 0.00% | 0.00% | 0.01% | 0.00% | 0.00% | 0.00% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | A | 0.01% | 99.98% | 0.01% | 0.01% | 99.97% | 0.01% | 0.01% | 0.01% |
| DdCBE_ | | | | | | | | | |
| replicate3 | | | | | | | | | |
| ND4- | T | 99.99% | 0.01% | 99.97% | 0.02% | 0.01% | 0.01% | 99.98% | 0.01% |
| DdCBE_ | | | | | | | | | |
| replicate3 | | | | | | | | | |
| ND4- | C | 0.01% | 0.00% | 0.01% | 99.97% | 0.01% | 99.98% | 0.01% | 99.98% |
| DdCBE_ | | | | | | | | | |
| replicate3 | | | | | | | | | |
| ND4- | G | 0.00% | 0.01% | 0.00% | 0.00% | 0.01% | 0.00% | 0.00% | 0.00% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | C | A | A | C | A | T | A | C |
| ND4- | A | 0.01% | 99.98% | 99.95% | 0.00% | 99.97% | 0.01% | 99.96% | 0.00% |
| DdCBE_ | | | | | | | | | |
| replicate1 | | | | | | | | | |
| ND4- | T | 0.02% | 0.00% | 0.01% | 0.01% | 0.02% | 99.99% | 0.01% | 0.02% |
| DdCBE_ | | | | | | | | | |
| replicate 1 | | | | | | | | | |
| ND4- | C | 99.97% | 0.01% | 0.02% | 99.98% | 0.01% | 0.01% | 0.02% | 99.98% |
| DdCBE_ | | | | | | | | | |
| replicate 1 | | | | | | | | | |
| ND4- | G | 0.00% | 0.01% | 0.01% | 0.00% | 0.01% | 0.00% | 0.01% | 0.00% |
| DdCBE_ | | | | | | | | | |
| replicate 1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | A | 0.01% | 99.96% | 99.94% | 0.01% | 99.98% | 0.00% | 99.97% | 0.01% |
| DdCBE_ | | | | | | | | | |
| replicate2 | | | | | | | | | |
| ND4- | T | 0.01% | 0.00% | 0.02% | 0.01% | 0.01% | 99.99% | 0.01% | 0.01% |
| DdCBE_ | | | | | | | | | |
| replicate2 | | | | | | | | | |
| ND4- | C | 99.98% | 0.03% | 0.03% | 99.98% | 0.00% | 0.00% | 0.01% | 99.99% |
| DdCBE_ | | | | | | | | | |
| replicate2 | | | | | | | | | |
| ND4- | G | 0.00% | 0.02% | 0.02% | 0.00% | 0.01% | 0.00% | 0.01% | 0.00% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | A | 0.01% | 99.97% | 99.96% | 0.00% | 99.98% | 0.00% | 99.97% | 0.00% |
| DdCBE_ | | | | | | | | | |
| replicate3 | | | | | | | | | |
| ND4- | T | 0.01% | 0.00% | 0.01% | 0.01% | 0.01% | 99.99% | 0.01% | 0.02% |
| DdCBE_ | | | | | | | | | |
| replicate3 | | | | | | | | | |
| ND4- | C | 99.98% | 0.01% | 0.02% | 99.99% | 0.00% | 0.01% | 0.01% | 99.98% |
| DdCBE_ | | | | | | | | | |
| replicate3 | | | | | | | | | |
| ND4- | G | 0.00% | 0.02% | 0.01% | 0.00% | 0.01% | 0.00% | 0.00% | 0.00% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | G | A | G | A | A | C | T | C |
| ND4- | A | 0.02% | 99.95% | 0.01% | 99.96% | 99.97% | 0.01% | 0.00% | 0.00% |
| DdCBE_ | | | | | | | | | |
| replicate 1 | | | | | | | | | |
| ND4- | T | 0.03% | 0.02% | 0.02% | 0.01% | 0.01% | 0.01% | 99.98% | 0.01% |
| DdCBE_ | | | | | | | | | |
| replicate1 | | | | | | | | | |
| ND4- | C | 0.00% | 0.02% | 0.00% | 0.02% | 0.00% | 99.98% | 0.01% | 99.99% |
| DdCBE_ | | | | | | | | | |
| replicate 1 | | | | | | | | | |
| ND4- | G | 99.93% | 0.01% | 99.97% | 0.01% | 0.02% | 0.00% | 0.00% | 0.00% |
| DdCBE_ | | | | | | | | | |
| replicate1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND4- | — | 0.01% | 0.00% | 0.00% | 0.00% | 0.00% | 0.00% | 0.00% | 0.00% |
| DdCBE_ | | | | | | | | | |
| replicate1 | | | | | | | | | |
| ND4- | A | 0.01% | 99.95% | 0.01% | 99.96% | 99.98% | 0.02% | 0.00% | 0.01% |
| DdCBE_ | | | | | | | | | |
| replicate2 | | | | | | | | | |
| ND4- | T | 0.04% | 0.02% | 0.03% | 0.01% | 0.01% | 0.01% | 99.99% | 0.00% |
| DdCBE_ | | | | | | | | | |
| replicate2 | | | | | | | | | |
| ND4 | C | 0.00% | 0.02% | 0.00% | 0.01% | 0.00% | 99.98% | 0.01% | 99.99% |
| DdCBE_ | | | | | | | | | |
| replicate2 | | | | | | | | | |
| ND4 | G | 99.93% | 0.02% | 99.96% | 0.02% | 0.01% | 0.00% | 0.00% | 0.00% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.01% | 0.00% | 0.00% | 0.00% | 0.00% | 0.00% | 0.00% | 0.00% |
| DdCBE_ | | | | | | | | | |
| replicate2 | | | | | | | | | |
| ND4 | A | 0.03% | 99.97% | 0.00% | 99.96% | 99.98% | 0.01% | 0.00% | 0.00% |
| DdCBE_ | | | | | | | | | |
| replicate3 | | | | | | | | | |
| ND4 | T | 0.03% | 0.01% | 0.01% | 0.01% | 0.01% | 0.01% | 99.98% | 0.01% |
| DdCBE_ | | | | | | | | | |
| replicate3 | | | | | | | | | |
| ND4- | C | 0.00% | 0.01% | 0.00% | 0.01% | 0.00% | 99.99% | 0.02% | 99.98% |
| DdCBE_ | | | | | | | | | |
| replicate3 | | | | | | | | | |
| ND4- | G | 99.93% | 0.01% | 99.98% | 0.01% | 0.01% | 0.00% | 0.00% | 0.00% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | | 0.01% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | C | G | T | T | C | T | C | C |
| ND4- | A | 0.00% | 0.25% | 0.00% | 0.00% | 0.01% | 0.01% | 0.01% | 0.02% |
| DdCBE_ | | | | | | | | | |
| replicate 1 | | | | | | | | | |
| ND4- | T | 0.02% | 0.00% | 99.96% | 99.98% | 0.01% | 99.98% | 0.02% | 0.01% |
| DdCBE_ | | | | | | | | | |
| replicate1 | | | | | | | | | |
| ND4- | C | 99.98% | 0.00% | 0.03% | 0.01% | 99.99% | 0.01% | 99.97% | 99.97% |
| DdCBE_ | | | | | | | | | |
| replicate1 | | | | | | | | | |
| ND4- | G | 0.00% | 99.75% | 0.01% | 0.00% | 0.00% | 0.00% | 0.00% | 0.00% |
| DdCBE_ | | | | | | | | | |
| replicate1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | A | 0.00% | 0.24% | 0.00% | 0.00% | 0.00% | 0.01% | 0.01% | 0.03% |
| DdCBE_ | | | | | | | | | |
| replicate2 | | | | | | | | | |
| ND4- | T | 0.03% | 0.01% | 99.97% | 99.99% | 0.00% | 99.97% | 0.03% | 0.01% |
| DdCBE_ | | | | | | | | | |
| replicate2 | | | | | | | | | |
| ND4- | C | 99.97% | 0.00% | 0.03% | 0.01% | 99.99% | 0.02% | 99.96% | 99.96% |
| DdCBE_ | | | | | | | | | |
| replicate2 | | | | | | | | | |
| ND4- | G | 0.00% | 99.75% | 0.00% | 0.00% | 0.00% | 0.00% | 0.00% | 0.00% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | A | 0.01% | 0.23% | 0.00% | 0.01% | 0.00% | 0.01% | 0.01% | 0.02% |
| DdCBE_ | | | | | | | | | |
| replicate3 | | | | | | | | | |
| ND4- | T | 0.02% | 0.00% | 99.96% | 99.99% | 0.00% | 99.99% | 0.01% | 0.01% |
| DdCBE_ | | | | | | | | | |
| replicate3 | | | | | | | | | |
| ND4- | C | 99.97% | 0.00% | 0.03% | 0.01% | 99.99% | 0.00% | 99.98% | 99.97% |
| DdCBE_ | | | | | | | | | |
| replicate3 | | | | | | | | | |
| ND4- | G | 0.00% | 99.76% | 0.00% | 0.00% | 0.00% | 0.00% | 0.00% | 0.00% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | T | C | C | T | A | C | T | T |
| ND4- | A | 0.01% | 0.01% | 0.01% | 0.01% | 99.97% | 0.01% | 0.01% | 0.01% |
| DdCBE_ | | | | | | | | | |
| replicate 1 | | | | | | | | | |
| ND4- | T | 99.98% | 0.01% | 0.02% | 99.98% | 0.01% | 0.01% | 99.98% | 99.97% |
| DdCBE_ | | | | | | | | | |
| replicate1 | | | | | | | | | |
| ND4- | C | 0.01% | 99.98% | 99.97% | 0.01% | 0.02% | 99.99% | 0.01% | 0.01% |
| DdCBE_ | | | | | | | | | |
| replicate 1 | | | | | | | | | |
| ND4- | G | 0.00% | 0.00% | 0.00% | 0.00% | 0.01% | 0.00% | 0.00% | 0.00% |
| DdCBE_ | | | | | | | | | |
| replicate 1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND4- | | 0.00% | 0.00% | 0.00% | 0.00% | 0.00% | 0.00% | 0.00% | 0.01% |
| DdCBE_ | | | | | | | | | |
| replicate 1 | | | | | | | | | |
| ND4- | A | 0.02% | 0.01% | 0.01% | 0.02% | 99.96% | 0.00% | 0.01% | 0.00% |
| DdCBE_ | | | | | | | | | |
| replicate2 | | | | | | | | | |
| ND4- | T | 99.97% | 0.00% | 0.02% | 99.97% | 0.01% | 0.00% | 99.99% | 99.98% |
| DdCBE_ | | | | | | | | | |
| replicate2 | | | | | | | | | |
| ND4- | C | 0.00% | 99.99% | 99.97% | 0.01% | 0.02% | 100.00% | 0.01% | 0.01% |
| DdCBE_ | | | | | | | | | |
| replicate2 | | | | | | | | | |
| ND4- | G | 0.01% | 0.00% | 0.01% | 0.00% | 0.01% | 0.00% | 0.00% | 0.00% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.01% |
| DdCBE_ | | | | | | | | | |
| replicate2 | | | | | | | | | |
| ND4- | A | 0.01% | 0.02% | 0.00% | 0.01% | 99.97% | 0.00% | 0.01% | 0.01% |
| DdCBE_ | | | | | | | | | |
| replicate3 | | | | | | | | | |
| ND4- | T | 99.97% | 0.01% | 0.02% | 99.98% | 0.01% | 0.01% | 99.98% | 99.96% |
| DdCBE_ | | | | | | | | | |
| replicate3 | | | | | | | | | |
| ND4- | C | 0.02% | 99.98% | 99.97% | 0.01% | 0.01% | 99.99% | 0.01% | 0.01% |
| DdCBE_ | | | | | | | | | |
| replicate3 | | | | | | | | | |
| ND4- | G | 0.00% | 0.00% | 0.00% | 0.00% | 0.01% | 0.00% | 0.00% | 0.00% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | — | 0.00% | 0.00% | 0.00% | 0.00% | 0.00% | 0.00% | 0.00% | 0.01% |
| DdCBE_ | | | | | | | | | |
| replicate3 | | | | | | | | | |
| Batch | Nucleotide | T | A | G | T | C | A | C | A |
| ND4- | A | 0.02% | 99.98% | 0.24% | 0.02% | 0.02% | 99.96% | 0.01% | 99.99% |
| DdCBE_ | | | | | | | | | |
| replicate1 | | | | | | | | | |
| ND4- | T | 99.97% | 0.00% | 0.01% | 99.95% | 0.03% | 0.01% | 0.01% | 0.00% |
| DdCBE_ | | | | | | | | | |
| replicate 1 | | | | | | | | | |
| ND4- | C | 0.01% | 0.00% | 0.00% | 0.03% | 99.95% | 0.02% | 99.98% | 0.00% |
| DdCBE_ | | | | | | | | | |
| replicate 1 | | | | | | | | | |
| ND4- | G | 0.00% | 0.02% | 99.76% | 0.00% | 0.00% | 0.01% | 0.00% | 0.01% |
| DdCBE_ | | | | | | | | | |
| replicate 1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | A | 0.01% | 99.97% | 0.23% | 0.02% | 0.03% | 99.96% | 0.00% | 99.99% |
| DdCBE_ | | | | | | | | | |
| replicate2 | | | | | | | | | |
| ND4- | T | 99.97% | 0.01% | 0.01% | 99.92% | 0.05% | 0.01% | 0.01% | 0.00% |
| DdCBE_ | | | | | | | | | |
| replicate2 | | | | | | | | | |
| ND4- | C | 0.01% | 0.00% | 0.00% | 0.06% | 99.92% | 0.01% | 99.98% | 0.00% |
| DdCBE_ | | | | | | | | | |
| replicate2 | | | | | | | | | |
| ND4- | G | 0.00% | 0.02% | 99.77% | 0.00% | 0.00% | 0.02% | 0.00% | 0.01% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | A | 0.01% | 99.99% | 0.21% | 0.02% | 0.02% | 99.97% | 0.01% | 99.98% |
| DdCBE_ | | | | | | | | | |
| replicate3 | | | | | | | | | |
| ND4- | T | 99.97% | 0.01% | 0.01% | 99.93% | 0.03% | 0.01% | 0.01% | 0.00% |
| DdCBE_ | | | | | | | | | |
| replicate3 | | | | | | | | | |
| ND4- | C | 0.02% | 0.00% | 0.00% | 0.05% | 99.95% | 0.00% | 99.98% | 0.00% |
| DdCBE_ | | | | | | | | | |
| replicate3 | | | | | | | | | |
| ND4- | G | 0.00% | 0.01% | 99.78% | 0.00% | 0.00% | 0.01% | 0.00% | 0.01% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | T | C | T | G | T | G | C | T |
| ND4- | A | 0.01% | 0.01% | 0.01% | 0.02% | 0.02% | 0.01% | 0.01% | 0.00% |
| DdCBE_ | | | | | | | | | |
| replicate 1 | | | | | | | | | |
| ND4- | T | 99.98% | 0.01% | 99.97% | 0.00% | 99.96% | 0.00% | 0.01% | 99.98% |
| DdCBE_ | | | | | | | | | |
| replicate1 | | | | | | | | | |
| ND4- | C | 0.01% | 99.97% | 0.01% | 0.00% | 0.01% | 0.00% | 99.98% | 0.01% |
| DdCBE_ | | | | | | | | | |
| replicate 1 | | | | | | | | | |
| ND4- | G | 0.00% | 0.01% | 0.00% | 99.98% | 0.01% | 99.98% | 0.00% | 0.00% |
| DdCBE_ | | | | | | | | | |
| replicate1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | A | 0.01% | 0.01% | 0.01% | 0.01% | 0.02% | 0.01% | 0.01% | 0.00% |
| DdCBE_ | | | | | | | | | |
| replicate2 | | | | | | | | | |
| ND4- | T | 99.98% | 0.01% | 99.98% | 0.00% | 99.96% | 0.01% | 0.01% | 99.99% |
| DdCBE_ | | | | | | | | | |
| replicate2 | | | | | | | | | |
| ND4 | C | 0.01% | 99.97% | 0.01% | 0.00% | 0.01% | 0.01% | 99.99% | 0.01% |
| DdCBE_ | | | | | | | | | |
| replicate2 | | | | | | | | | |
| ND4 | G | 0.00% | 0.00% | 0.00% | 99.99% | 0.01% | 99.97% | 0.00% | 0.00% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4 | A | 0.01% | 0.01% | 0.01% | 0.01% | 0.01% | 0.01% | 0.01% | 0.01% |
| DdCBE_ | | | | | | | | | |
| replicate3 | | | | | | | | | |
| ND4 | T | 99.97% | 0.01% | 99.98% | 0.01% | 99.95% | 0.01% | 0.01% | 99.98% |
| DdCBE_ | | | | | | | | | |
| replicate3 | | | | | | | | | |
| ND4- | C | 0.01% | 99.97% | 0.01% | 0.00% | 0.02% | 0.01% | 99.98% | 0.01% |
| DdCBE_ | | | | | | | | | |
| replicate3 | | | | | | | | | |
| ND4- | G | 0.00% | 0.01% | 0.00% | 99.98% | 0.01% | 99.97% | 0.00% | 0.00% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | T | G | A | T | C | A | A | A |
| ND4- | A | 0.00% | 47.28% | 99.97% | 0.01% | 0.01% | 99.96% | 99.98% | 99.98% |
| DdCBE_ | | | | | | | | | |
| replicate 1 | | | | | | | | | |
| ND4- | T | 99.98% | 0.01% | 0.02% | 99.98% | 0.37% | 0.01% | 0.01% | 0.01% |
| DdCBE_ | | | | | | | | | |
| replicate1 | | | | | | | | | |
| ND4- | C | 0.01% | 0.00% | 0.00% | 0.01% | 99.61% | 0.00% | 0.00% | 0.00% |
| DdCBE_ | | | | | | | | | |
| replicate1 | | | | | | | | | |
| ND4- | G | 0.00% | 52.70% | 0.01% | 0.00% | 0.00% | 0.02% | 0.01% | 0.01% |
| DdCBE_ | | | | | | | | | |
| replicate1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND4- | — | 0.00% | 0.01% | 0.00% | 0.00% | 0.00% | 0.01% | 0.00% | 0.00% |
| DdCBE_ | | | | | | | | | |
| replicate1 | | | | | | | | | |
| ND4- | A | 0.01% | 47.37% | 99.98% | 0.01% | 0.01% | 99.97% | 99.99% | 99.98% |
| DdCBE_ | | | | | | | | | |
| replicate2 | | | | | | | | | |
| ND4- | T | 99.98% | 0.00% | 0.01% | 99.99% | 0.43% | 0.00% | 0.00% | 0.01% |
| DdCBE_ | | | | | | | | | |
| replicate2 | | | | | | | | | |
| ND4- | C | 0.01% | 0.00% | 0.01% | 0.01% | 99.56% | 0.00% | 0.00% | 0.00% |
| DdCBE_ | | | | | | | | | |
| replicate2 | | | | | | | | | |
| ND4- | G | 0.00% | 52.62% | 0.00% | 0.00% | 0.00% | 0.01% | 0.01% | 0.01% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.01% | 0.00% | 0.00% | 0.00% | 0.01% | 0.00% | 0.00% |
| DdCBE_ | | | | | | | | | |
| replicate2 | | | | | | | | | |
| ND4- | A | 0.01% | 43.55% | 99.97% | 0.01% | 0.00% | 99.97% | 99.97% | 99.98% |
| DdCBE_ | | | | | | | | | |
| replicate3 | | | | | | | | | |
| ND4- | T | 99.98% | 0.00% | 0.02% | 99.99% | 0.36% | 0.00% | 0.01% | 0.00% |
| DdCBE_ | | | | | | | | | |
| replicate3 | | | | | | | | | |
| ND4- | C | 0.01% | 0.01% | 0.00% | 0.01% | 99.64% | 0.01% | 0.01% | 0.00% |
| DdCBE_ | | | | | | | | | |
| replicate3 | | | | | | | | | |
| ND4- | G | 0.00% | 56.43% | 0.01% | 0.00% | 0.00% | 0.01% | 0.02% | 0.01% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | — | 0.00% | 0.00% | 0.00% | 0.00% | 0.00% | 0.01% | 0.00% | 0.00% |
| DdCBE_ | | | | | | | | | |
| replicate3 | | | | | | | | | |
| Batch | Nucleotide | A | C | A | G | G | A | C | T |
| ND4- | A | 99.96% | 0.01% | 99.96% | 0.01% | 0.01% | 99.93% | 0.01% | 0.01% |
| DdCBE_ | | | | | | | | | |
| replicate 1 | | | | | | | | | |
| ND4- | T | 0.00% | 0.01% | 0.01% | 0.02% | 0.01% | 0.00% | 0.01% | 99.97% |
| DdCBE_ | | | | | | | | | |
| replicate1 | | | | | | | | | |
| ND4- | C | 0.03% | 99.98% | 0.00% | 0.00% | 0.00% | 0.05% | 99.98% | 0.02% |
| DdCBE_ | | | | | | | | | |
| replicate 1 | | | | | | | | | |
| ND4- | G | 0.01% | 0.00% | 0.03% | 99.97% | 99.98% | 0.02% | 0.00% | 0.00% |
| DdCBE_ | | | | | | | | | |
| replicate 1 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND4- | | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND4- | A | 99.95% | 0.01% | 99.97% | 0.01% | 0.01% | 99.94% | 0.00% | 0.01% |
| DdCBE_ | | | | | | | | | |
| replicate2 | | | | | | | | | |
| ND4- | T | 0.01% | 0.01% | 0.01% | 0.02% | 0.00% | 0.01% | 0.01% | 99.98% |
| DdCBE_ | | | | | | | | | |
| replicate2 | | | | | | | | | |
| ND4- | C | 0.02% | 99.98% | 0.01% | 0.00% | 0.01% | 0.05% | 99.99% | 0.01% |
| DdCBE_ | | | | | | | | | |
| replicate2 | | | | | | | | | |
| ND4- | G | 0.01% | 0.00% | 0.01% | 99.97% | 99.98% | 0.00% | 0.00% | 0.00% |
| DdCBE_ | | | | | | | | | |
| replicate2 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND4- | A | 99.96% | 0.01% | 99.97% | 0.02% | 0.01% | 99.92% | 0.00% | 0.01% |
| DdCBE_ | | | | | | | | | |
| replicate3 | | | | | | | | | |
| ND4- | T | 0.00% | 0.01% | 0.00% | 0.01% | 0.01% | 0.01% | 0.01% | 99.98% |
| DdCBE_ | | | | | | | | | |
| replicate3 | | | | | | | | | |
| ND4- | C | 0.03% | 99.98% | 0.00% | 0.00% | 0.00% | 0.06% | 99.99% | 0.02% |
| DdCBE_ | | | | | | | | | |
| replicate3 | | | | | | | | | |
| ND4- | G | 0.01% | 0.00% | 0.02% | 99.97% | 99.98% | 0.01% | 0.00% | 0.00% |
| DdCBE_ | | | | | | | | | |
| replicate3 | | | | | | | | | |
| ND4- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND4- | — | 0.00% | 0.01% | 0.01% | 0.00% | 0.00% | 0.00% | 0.00% | 0.00% |
| DdCBE_ | | | | | | | | | |
| replicate3 | | | | | | | | | |

| Batch | Nucleotide | G | C |
|---|---|---|---|
| ND4- | A | 0.02% | 0.01% |
| DdCBE_ | | | |
| replicate1 | | | |
| ND4- | T | 0.01% | 0.01% |
| DdCBE_ | | | |
| replicate 1 | | | |
| ND4- | C | 0.01% | 99.8% |
| DdCBE_ | | | |
| replicate 1 | | | |
| ND4- | G | 99.96% | 0.00% |
| DdCBE_ | | | |
| replicate 1 | | | |
| ND4- | N | 0.00% | 0.00% |
| DdCBE_ | | | |
| replicate1 | | | |
| ND4- | — | 0.00% | 0.00% |
| DdCBE_ | | | |
| replicate1 | | | |
| ND4- | A | 0.01% | 0.00% |
| DdCBE_ | | | |
| replicate2 | | | |
| ND4- | T | 0.01% | 0.01% |
| DdCBE_ | | | |
| replicate2 | | | |
| ND4- | C | 0.00% | 99.99% |
| DdCBE_ | | | |
| replicate2 | | | |
| ND4- | G | 99.98% | 0.00% |
| DdCBE_ | | | |
| replicate2 | | | |
| ND4- | N | 0.00% | 0.00% |
| DdCBE_ | | | |
| replicate2 | | | |
| ND4- | — | 0.00% | 0.00% |
| DdCBE_ | | | |
| replicate2 | | | |
| ND4- | A | 0.02% | 0.01% |
| DdCBE_ | | | |
| replicate3 | | | |
| ND4- | T | 0.01% | 0.00% |
| DdCBE_ | | | |
| replicate3 | | | |
| ND4- | C | 0.00% | 99.99% |
| DdCBE_ | | | |
| replicate3 | | | |
| ND4- | G | 99.97% | 0.00% |
| DdCBE_ | | | |
| replicate3 | | | |
| ND4- | N | 0.00% | 0.00% |
| DdCBE_ | | | |
| replicate3 | | | |
| ND4- | — | 0.00% | 0.00% |
| DdCBE_ | | | |
| replicate3 | | | |

TABLE 12F
Base Percentages at TALE Binding Sites-ND2-DdCBE

| Batch | Nucleotide | C | C | A | A | A | C | C | C |
|---|---|---|---|---|---|---|---|---|---|
| ND2- | A | 0.01% | 0.01% | 99.98% | 99.99% | 99.97% | 0.02% | 0.02% | 0.01% |
| DdCBE_ | | | | | | | | | |
| replicate 1 | | | | | | | | | |
| ND2- | T | 0.02% | 0.01% | 0.00% | 0.00% | 0.00% | 0.00% | 0.01% | 0.04% |
| DdCBE_ | | | | | | | | | |
| replicate 1 | | | | | | | | | |
| ND2- | C | 99.97% | 99.97% | 0.00% | 0.00% | 0.01% | 99.97% | 99.97% | 99.95% |
| DdCBE_ | | | | | | | | | |
| replicate1 | | | | | | | | | |
| ND2- | G | 0.00% | 0.00% | 0.02% | 0.01% | 0.01% | 0.00% | 0.00% | 0.00% |
| DdCBE_ | | | | | | | | | |
| replicate 1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | — | 0.00% | 0.00% | 0.00% | 0.00% | 0.01% | 0.00% | 0.00% | 0.00% |
| DdCBE_ | | | | | | | | | |
| replicate1 | | | | | | | | | |
| ND2- | A | 0.00% | 0.01% | 99.98% | 99.97% | 99.98% | 0.03% | 0.00% | 0.00% |
| DdCBE_ | | | | | | | | | |
| replicate2 | | | | | | | | | |
| ND2- | T | 0.01% | 0.02% | 0.00% | 0.00% | 0.00% | 0.00% | 0.02% | 0.04% |
| DdCBE_ | | | | | | | | | |
| replicate2 | | | | | | | | | |
| ND2- | C | 99.98% | 99.96% | 0.01% | 0.02% | 0.00% | 99.96% | 99.98% | 99.95% |
| DdCBE_ | | | | | | | | | |
| replicate2 | | | | | | | | | |
| ND2- | G | 0.00% | 0.00% | 0.02% | 0.01% | 0.01% | 0.00% | 0.00% | 0.00% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 0.00% | 0.01% | 99.99% | 99.98% | 99.98% | 0.02% | 0.01% | 0.01% |
| DdCBE_ | | | | | | | | | |
| replicate3 | | | | | | | | | |
| ND2- | T | 0.01% | 0.01% | 0.00% | 0.00% | 0.00% | 0.02% | 0.01% | 0.03% |
| DdCBE_ | | | | | | | | | |
| replicate3 | | | | | | | | | |
| ND2- | C | 99.99% | 99.97% | 0.00% | 0.01% | 0.01% | 99.96% | 99.99% | 99.96% |
| DdCBE_ | | | | | | | | | |
| replicate3 | | | | | | | | | |
| ND2- | G | 0.00% | 0.00% | 0.01% | 0.01% | 0.01% | 0.00% | 0.00% | 0.00% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.00% | 0.01% | 0.00% | 0.00% | 0.00% |
| DdCBE_ | | | | | | | | | |
| replicate3 | | | | | | | | | |
| Batch | Nucleotide | A | G | C | A | T | A | C | T |
| ND2- | A | 99.98% | 0.02% | 0.01% | 99.98% | 0.01% | 99.97% | 0.00% | 0.01% |
| DdCBE_ | | | | | | | | | |
| replicate1 | | | | | | | | | |
| ND2- | T | 0.00% | 0.01% | 0.02% | 0.01% | 99.97% | 0.01% | 0.01% | 99.97% |
| DdCBE_ | | | | | | | | | |
| replicate1 | | | | | | | | | |
| ND2- | C | 0.00% | 0.00% | 99.97% | 0.00% | 0.01% | 0.01% | 99.99% | 0.02% |
| DdCBE_ | | | | | | | | | |
| replicate1 | | | | | | | | | |
| ND2- | G | 0.02% | 99.97% | 0.00% | 0.01% | 0.00% | 0.01% | 0.00% | 0.01% |
| DdCBE_ | | | | | | | | | |
| replicate1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | A | 99.98% | 0.01% | 0.00% | 99.97% | 0.00% | 99.98% | 0.01% | 0.01% |
| DdCBE_ | | | | | | | | | |
| replicate2 | | | | | | | | | |
| ND2- | T | 0.00% | 0.01% | 0.02% | 0.01% | 99.98% | 0.01% | 0.00% | 99.97% |
| DdCBE_ | | | | | | | | | |
| replicate2 | | | | | | | | | |
| ND2- | C | 0.00% | 0.01% | 99.97% | 0.00% | 0.01% | 0.00% | 99.98% | 0.01% |
| DdCBE_ | | | | | | | | | |
| replicate2 | | | | | | | | | |
| ND2- | G | 0.01% | 99.97% | 0.00% | 0.02% | 0.01% | 0.00% | 0.00% | 0.01% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 99.98% | 0.01% | 0.01% | 99.96% | 0.01% | 99.97% | 0.02% | 0.01% |
| DdCBE_ | | | | | | | | | |
| replicate3 | | | | | | | | | |
| ND2- | T | 0.00% | 0.02% | 0.02% | 0.02% | 99.99% | 0.01% | 0.00% | 99.97% |
| DdCBE_ | | | | | | | | | |
| replicate3 | | | | | | | | | |
| ND2- | C | 0.00% | 0.00% | 99.98% | 0.00% | 0.00% | 0.01% | 99.98% | 0.01% |
| DdCBE_ | | | | | | | | | |
| replicate3 | | | | | | | | | |
| ND2- | G | 0.01% | 99.97% | 0.00% | 0.02% | 0.00% | 0.01% | 0.00% | 0.01% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | A | G | G | A | T | G | A | A |
| ND2- | A | 100.00% | 0.29% | 4.82% | 99.95% | 0.00% | 0.05% | 99.97% | 99.94% |
| DdCBE_ | | | | | | | | | |
| replicate 1 | | | | | | | | | |
| ND2- | T | 0.00% | 0.02% | 0.01% | 0.01% | 99.99% | 0.02% | 0.01% | 0.02% |
| DdCBE_ | | | | | | | | | |
| replicate 1 | | | | | | | | | |
| ND2- | C | 0.00% | 0.00% | 0.00% | 0.01% | 0.01% | 0.00% | 0.00% | 0.01% |
| DdCBE_ | | | | | | | | | |
| replicate 1 | | | | | | | | | |
| ND2- | G | 0.00% | 99.69% | 95.18% | 0.02% | 0.00% | 99.93% | 0.02% | 0.02% |
| DdCBE_ | | | | | | | | | |
| replicate 1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | A | 99.99% | 0.35% | 4.74% | 99.97% | 0.00% | 0.04% | 99.98% | 99.96% |
| DdCBE_ | | | | | | | | | |
| replicate2 | | | | | | | | | |
| ND2- | T | 0.00% | 0.02% | 0.00% | 0.01% | 99.98% | 0.01% | 0.01% | 0.01% |
| DdCBE_ | | | | | | | | | |
| replicate2 | | | | | | | | | |
| ND2- | C | 0.00% | 0.00% | 0.01% | 0.02% | 0.01% | 0.00% | 0.00% | 0.01% |
| DdCBE_ | | | | | | | | | |
| replicate2 | | | | | | | | | |
| ND2- | G | 0.01% | 99.64% | 95.25% | 0.00% | 0.00% | 99.95% | 0.01% | 0.02% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 99.99% | 0.33% | 4.37% | 99.98% | 0.01% | 0.05% | 99.97% | 99.98% |
| DdCBE_ | | | | | | | | | |
| replicate3 | | | | | | | | | |
| ND2- | T | 0.01% | 0.02% | 0.01% | 0.00% | 99.97% | 0.01% | 0.01% | 0.01% |
| DdCBE_ | | | | | | | | | |
| replicate3 | | | | | | | | | |
| ND2- | C | 0.00% | 0.01% | 0.01% | 0.01% | 0.01% | 0.01% | 0.00% | 0.00% |
| DdCBE_ | | | | | | | | | |
| replicate3 | | | | | | | | | |
| ND2- | G | 0.01% | 99.65% | 95.61% | 0.01% | 0.01% | 99.93% | 0.02% | 0.00% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | G | T | A | C | A | A | C | C |
| ND2- | A | 0.01% | 0.01% | 99.98% | 0.01% | 99.99% | 99.98% | 0.01% | 0.02% |
| DdCBE_ | | | | | | | | | |
| replicate 1 | | | | | | | | | |
| ND2- | T | 0.00% | 99.94% | 0.01% | 0.02% | 0.00% | 0.01% | 0.02% | 0.01% |
| DdCBE_ | | | | | | | | | |
| replicate 1 | | | | | | | | | |
| ND2- | C | 0.00% | 0.05% | 0.00% | 99.97% | 0.01% | 0.00% | 99.97% | 99.98% |
| DdCBE_ | | | | | | | | | |
| replicate 1 | | | | | | | | | |
| ND2- | G | 99.98% | 0.01% | 0.01% | 0.00% | 0.01% | 0.01% | 0.00% | 0.00% |
| DdCBE_ | | | | | | | | | |
| replicate 1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND2- | A | 0.02% | 0.01% | 99.97% | 0.00% | 99.99% | 99.98% | 0.00% | 0.02% |
| DdCBE_ | | | | | | | | | |
| replicate2 | | | | | | | | | |
| ND2- | T | 0.02% | 99.95% | 0.01% | 0.02% | 0.00% | 0.01% | 0.02% | 0.02% |
| DdCBE_ | | | | | | | | | |
| replicate2 | | | | | | | | | |
| ND2- | C | 0.00% | 0.05% | 0.01% | 99.98% | 0.00% | 0.00% | 99.98% | 99.96% |
| DdCBE_ | | | | | | | | | |
| replicate2 | | | | | | | | | |
| ND2- | G | 99.96% | 0.00% | 0.01% | 0.00% | 0.01% | 0.00% | 0.00% | 0.00% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 0.04% | 0.01% | 99.97% | 0.01% | 99.98% | 99.97% | 0.00% | 0.02% |
| DdCBE_ | | | | | | | | | |
| replicate3 | | | | | | | | | |
| ND2- | T | 0.01% | 99.95% | 0.02% | 0.01% | 0.00% | 0.01% | 0.00% | 0.01% |
| DdCBE_ | | | | | | | | | |
| replicate3 | | | | | | | | | |
| ND2- | C | 0.00% | 0.04% | 0.00% | 99.98% | 0.01% | 0.01% | 99.99% | 99.97% |
| DdCBE_ | | | | | | | | | |
| replicate3 | | | | | | | | | |
| ND2- | G | 99.95% | 0.00% | 0.01% | 0.00% | 0.01% | 0.01% | 0.00% | 0.00% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | A | G | C | T | A | C | G | C |
| ND2- | A | 99.97% | 0.18% | 0.00% | 0.00% | 99.98% | 0.01% | 0.03% | 0.01% |
| DdCBE_ | | | | | | | | | |
| replicate 1 | | | | | | | | | |
| ND2- | T | 0.01% | 0.02% | 0.01% | 99.98% | 0.01% | 0.02% | 0.01% | 0.01% |
| DdCBE_ | | | | | | | | | |
| replicate 1 | | | | | | | | | |
| ND2- | C | 0.00% | 0.00% | 99.99% | 0.02% | 0.01% | 99.98% | 0.01% | 99.99% |
| DdCBE_ | | | | | | | | | |
| replicate1 | | | | | | | | | |
| ND2- | G | 0.01% | 99.80% | 0.00% | 0.00% | 0.01% | 0.00% | 99.95% | 0.00% |
| DdCBE_ | | | | | | | | | |
| replicate 1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND2- | A | 99.97% | 0.20% | 0.01% | 0.00% | 99.99% | 0.00% | 0.05% | 0.01% |
| DdCBE_ | | | | | | | | | |
| replicate2 | | | | | | | | | |
| ND2- | T | 0.02% | 0.02% | 0.02% | 99.99% | 0.00% | 0.01% | 0.01% | 0.01% |
| DdCBE_ | | | | | | | | | |
| replicate2 | | | | | | | | | |
| ND2- | C | 0.00% | 0.00% | 99.98% | 0.01% | 0.00% | 99.99% | 0.01% | 99.98% |
| DdCBE_ | | | | | | | | | |
| replicate2 | | | | | | | | | |
| ND2- | G | 0.00% | 99.78% | 0.00% | 0.00% | 0.00% | 0.00% | 99.93% | 0.00% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 99.96% | 0.14% | 0.00% | 0.00% | 99.98% | 0.00% | 0.08% | 0.00% |
| DdCBE_ | | | | | | | | | |
| replicate3 | | | | | | | | | |
| ND2- | T | 0.02% | 0.03% | 0.00% | 99.99% | 0.01% | 0.01% | 0.02% | 0.01% |
| DdCBE_ | | | | | | | | | |
| replicate3 | | | | | | | | | |
| ND2- | C | 0.01% | 0.00% | 99.99% | 0.01% | 0.00% | 99.99% | 0.01% | 99.98% |
| DdCBE_ | | | | | | | | | |
| replicate3 | | | | | | | | | |
| ND2- | G | 0.02% | 99.83% | 0.00% | 0.00% | 0.01% | 0.00% | 99.90% | 0.00% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | C | C | T | C | A | A | T | T |
| ND2- | A | 0.03% | 0.01% | 0.01% | 0.00% | 99.96% | 99.98% | 0.01% | 0.01% |
| DdCBE_ | | | | | | | | | |
| replicate1 | | | | | | | | | |
| ND2- | T | 0.02% | 0.01% | 99.98% | 0.31% | 0.01% | 0.00% | 99.99% | 99.98% |
| DdCBE_ | | | | | | | | | |
| replicate1 | | | | | | | | | |
| ND2- | C | 99.96% | 99.99% | 0.01% | 99.68% | 0.00% | 0.02% | 0.00% | 0.01% |
| DdCBE_ | | | | | | | | | |
| replicate1 | | | | | | | | | |
| ND2- | G | 0.00% | 0.00% | 0.00% | 0.00% | 0.03% | 0.00% | 0.00% | 0.00% |
| DdCBE_ | | | | | | | | | |
| replicate1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | — | 0.00% | 0.00% | 0.00% | 0.00% | 0.00% | 0.00% | 0.00% | 0.01% |
| DdCBE_ | | | | | | | | | |
| replicate 1 | | | | | | | | | |
| ND2- | A | 0.01% | 0.01% | 0.00% | 0.00% | 99.95% | 99.98% | 0.01% | 0.01% |
| DdCBE_ | | | | | | | | | |
| replicate2 | | | | | | | | | |
| ND2- | T | 0.03% | 0.01% | 99.97% | 0.33% | 0.01% | 0.01% | 99.98% | 99.97% |
| DdCBE_ | | | | | | | | | |
| replicate2 | | | | | | | | | |
| ND2- | C | 99.95% | 99.98% | 0.02% | 99.66% | 0.00% | 0.01% | 0.01% | 0.02% |
| DdCBE_ | | | | | | | | | |
| replicate2 | | | | | | | | | |
| ND2- | G | 0.00% | 0.00% | 0.01% | 0.00% | 0.04% | 0.00% | 0.00% | 0.00% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 0.02% | 0.01% | 0.01% | 0.00% | 99.96% | 99.97% | 0.01% | 0.01% |
| DdCBE_ | | | | | | | | | |
| replicate3 | | | | | | | | | |
| ND2- | T | 0.03% | 0.01% | 99.98% | 0.33% | 0.01% | 0.01% | 99.98% | 99.98% |
| DdCBE_ | | | | | | | | | |
| replicate3 | | | | | | | | | |
| ND2- | C | 99.96% | 99.98% | 0.01% | 99.67% | 0.00% | 0.02% | 0.01% | 0.02% |
| DdCBE_ | | | | | | | | | |
| replicate3 | | | | | | | | | |
| ND2- | G | 0.00% | 0.00% | 0.01% | 0.00% | 0.03% | 0.01% | 0.00% | 0.00% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | T | A | A | T | A | G | C | A |
| ND2- | A | 0.00% | 99.98% | 99.96% | 0.01% | 99.98% | 0.02% | 0.01% | 99.97% |
| DdCBE_ | | | | | | | | | |
| replicate 1 | | | | | | | | | |
| ND2- | T | 99.98% | 0.01% | 0.02% | 99.82% | 0.01% | 0.00% | 0.00% | 0.00% |
| DdCBE_ | | | | | | | | | |
| replicate 1 | | | | | | | | | |
| ND2- | C | 0.01% | 0.00% | 0.01% | 0.18% | 0.00% | 0.00% | 99.98% | 0.01% |
| DdCBE_ | | | | | | | | | |
| replicate 1 | | | | | | | | | |
| ND2- | G | 0.01% | 0.01% | 0.01% | 0.00% | 0.02% | 99.97% | 0.01% | 0.02% |
| DdCBE_ | | | | | | | | | |
| replicate 1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | A | 0.01% | 99.99% | 99.96% | 0.01% | 99.97% | 0.01% | 0.01% | 99.97% |
| DdCBE_ | | | | | | | | | |
| replicate2 | | | | | | | | | |
| ND2- | T | 99.97% | 0.00% | 0.02% | 99.79% | 0.00% | 0.01% | 0.02% | 0.01% |
| DdCBE_ | | | | | | | | | |
| replicate2 | | | | | | | | | |
| ND2- | C | 0.02% | 0.00% | 0.00% | 0.20% | 0.00% | 0.01% | 99.97% | 0.00% |
| DdCBE_ | | | | | | | | | |
| replicate2 | | | | | | | | | |
| ND2- | G | 0.00% | 0.01% | 0.01% | 0.00% | 0.02% | 99.97% | 0.00% | 0.02% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 0.00% | 99.99% | 99.96% | 0.00% | 99.98% | 0.01% | 0.00% | 99.97% |
| DdCBE_ | | | | | | | | | |
| replicate3 | | | | | | | | | |
| ND2- | T | 99.98% | 0.00% | 0.02% | 99.84% | 0.01% | 0.01% | 0.02% | 0.00% |
| DdCBE_ | | | | | | | | | |
| replicate3 | | | | | | | | | |
| ND2- | C | 0.01% | 0.00% | 0.01% | 0.16% | 0.00% | 0.01% | 99.97% | 0.01% |
| DdCBE_ | | | | | | | | | |
| replicate3 | | | | | | | | | |
| ND2- | G | 0.00% | 0.01% | 0.01% | 0.00% | 0.01% | 99.97% | 0.01% | 0.02% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.01% | 0.00% | 0.00% | 0.00% | 0.00% | 0.00% |
| DdCBE_ | | | | | | | | | |
| replicate3 | | | | | | | | | |
| Batch | Nucleotide | C | T | A | A | C | A | T | A |
| ND2- | A | 0.00% | 0.01% | 99.97% | 99.96% | 0.01% | 99.96% | 0.01% | 99.97% |
| DdCBE_ | | | | | | | | | |
| replicate 1 | | | | | | | | | |
| ND2- | T | 0.02% | 99.98% | 0.00% | 0.01% | 0.01% | 0.02% | 99.98% | 0.01% |
| DdCBE_ | | | | | | | | | |
| replicate 1 | | | | | | | | | |
| ND2- | C | 99.98% | 0.01% | 0.01% | 0.01% | 99.98% | 0.00% | 0.01% | 0.01% |
| DdCBE_ | | | | | | | | | |
| replicate 1 | | | | | | | | | |
| ND2- | G | 0.01% | 0.00% | 0.02% | 0.01% | 0.00% | 0.01% | 0.00% | 0.00% |
| DdCBE_ | | | | | | | | | |
| replicate 1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND2- | — | 0.00% | 0.00% | 0.00% | 0.01% | 0.00% | 0.00% | 0.00% | 0.00% |
| DdCBE_ | | | | | | | | | |
| replicate1 | | | | | | | | | |
| ND2- | A | 0.01% | 0.00% | 99.96% | 99.98% | 0.01% | 99.97% | 0.00% | 99.98% |
| DdCBE_ | | | | | | | | | |
| replicate2 | | | | | | | | | |
| ND2- | T | 0.02% | 99.98% | 0.00% | 0.00% | 0.01% | 0.01% | 99.99% | 0.00% |
| DdCBE_ | | | | | | | | | |
| replicate2 | | | | | | | | | |
| ND2- | C | 99.97% | 0.02% | 0.00% | 0.01% | 99.98% | 0.00% | 0.00% | 0.01% |
| DdCBE_ | | | | | | | | | |
| replicate2 | | | | | | | | | |
| ND2- | G | 0.00% | 0.00% | 0.03% | 0.00% | 0.00% | 0.01% | 0.00% | 0.01% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.01% | 0.00% | 0.00% | 0.00% | 0.00% |
| DdCBE_ | | | | | | | | | |
| replicate2 | | | | | | | | | |
| ND2- | A | 0.01% | 0.01% | 99.98% | 99.96% | 0.00% | 99.97% | 0.00% | 99.97% |
| DdCBE_ | | | | | | | | | |
| replicate3 | | | | | | | | | |
| ND2- | T | 0.02% | 99.98% | 0.00% | 0.01% | 0.01% | 0.02% | 100.00% | 0.01% |
| DdCBE_ | | | | | | | | | |
| replicate3 | | | | | | | | | |
| ND2- | C | 99.97% | 0.01% | 0.00% | 0.02% | 99.99% | 0.00% | 0.00% | 0.01% |
| DdCBE_ | | | | | | | | | |
| replicate3 | | | | | | | | | |
| ND2- | G | 0.00% | 0.00% | 0.02% | 0.01% | 0.00% | 0.00% | 0.00% | 0.01% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.01% | 0.00% | 0.00% | 0.00% | 0.00% |
| DdCBE_ | | | | | | | | | |
| replicate3 | | | | | | | | | |
| Batch | Nucleotide | A | A | A | A | T | C | T | T |
| ND2- | A | 99.97% | 99.96% | 99.89% | 99.92% | 0.00% | 0.00% | 0.00% | 0.00% |
| DdCBE_ | | | | | | | | | |
| replicate 1 | | | | | | | | | |
| ND2- | T | 0.02% | 0.02% | 0.02% | 0.00% | 99.99% | 0.00% | 100.00% | 99.98% |
| DdCBE_ | | | | | | | | | |
| replicate 1 | | | | | | | | | |
| ND2- | C | 0.00% | 0.01% | 0.07% | 0.06% | 0.01% | 99.99% | 0.00% | 0.02% |
| DdCBE_ | | | | | | | | | |
| replicate1 | | | | | | | | | |
| ND2- | G | 0.01% | 0.02% | 0.02% | 0.02% | 0.00% | 0.01% | 0.00% | 0.00% |
| DdCBE_ | | | | | | | | | |
| replicate 1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND2- | A | 99.96% | 99.97% | 99.87% | 99.95% | 0.00% | 0.00% | 0.00% | 0.00% |
| DdCBE_ | | | | | | | | | |
| replicate2 | | | | | | | | | |
| ND2- | T | 0.01% | 0.01% | 0.02% | 0.00% | 99.99% | 0.01% | 99.99% | 99.98% |
| DdCBE_ | | | | | | | | | |
| replicate2 | | | | | | | | | |
| ND2- | C | 0.01% | 0.00% | 0.10% | 0.03% | 0.01% | 99.98% | 0.00% | 0.01% |
| DdCBE_ | | | | | | | | | |
| replicate2 | | | | | | | | | |
| ND2- | G | 0.02% | 0.01% | 0.01% | 0.02% | 0.00% | 0.01% | 0.00% | 0.00% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.01% |
| DdCBE_ | | | | | | | | | |
| replicate2 | | | | | | | | | |
| ND2- | A | 99.96% | 99.95% | 99.90% | 99.96% | 0.00% | 0.01% | 0.00% | 0.00% |
| DdCBE_ | | | | | | | | | |
| replicate3 | | | | | | | | | |
| ND2- | T | 0.01% | 0.02% | 0.01% | 0.01% | 99.99% | 0.01% | 99.99% | 99.97% |
| DdCBE_ | | | | | | | | | |
| replicate3 | | | | | | | | | |
| ND2- | C | 0.01% | 0.00% | 0.08% | 0.03% | 0.01% | 99.98% | 0.01% | 0.02% |
| DdCBE_ | | | | | | | | | |
| replicate3 | | | | | | | | | |
| ND2- | G | 0.01% | 0.03% | 0.01% | 0.01% | 0.00% | 0.00% | 0.00% | 0.00% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.00% | 0.00% | 0.00% | 0.00% | 0.01% |
| DdCBE_ | | | | | | | | | |
| replicate3 | | | | | | | | | |
| Batch | Nucleotide | A | C | C | C | A | C | A | T |
| ND2- | A | 99.98% | 0.01% | 0.00% | 0.02% | 99.96% | 0.01% | 99.99% | 0.00% |
| DdCBE_ | | | | | | | | | |
| replicate1 | | | | | | | | | |
| ND2- | T | 0.01% | 0.01% | 0.02% | 0.01% | 0.02% | 0.74% | 0.01% | 99.99% |
| DdCBE_ | | | | | | | | | |
| replicate1 | | | | | | | | | |
| ND2- | C | 0.00% | 99.98% | 99.97% | 99.96% | 0.00% | 99.25% | 0.00% | 0.01% |
| DdCBE_ | | | | | | | | | |
| replicate1 | | | | | | | | | |
| ND2- | G | 0.01% | 0.00% | 0.01% | 0.01% | 0.02% | 0.00% | 0.00% | 0.00% |
| DdCBE_ | | | | | | | | | |
| replicate1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | — | 0.01% | 0.00% | 0.00% | 0.00% | 0.01% | 0.00% | 0.00% | 0.00% |
| DdCBE_ | | | | | | | | | |
| replicate 1 | | | | | | | | | |
| ND2- | A | 99.98% | 0.01% | 0.00% | 0.02% | 99.97% | 0.00% | 99.97% | 0.00% |
| DdCBE_ | | | | | | | | | |
| replicate2 | | | | | | | | | |
| ND2- | T | 0.01% | 0.01% | 0.01% | 0.02% | 0.01% | 0.79% | 0.01% | 99.99% |
| DdCBE_ | | | | | | | | | |
| replicate2 | | | | | | | | | |
| ND2- | C | 0.00% | 99.98% | 99.98% | 99.95% | 0.01% | 99.20% | 0.01% | 0.01% |
| DdCBE_ | | | | | | | | | |
| replicate2 | | | | | | | | | |
| ND2- | G | 0.01% | 0.00% | 0.00% | 0.00% | 0.01% | 0.00% | 0.02% | 0.00% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 99.98% | 0.00% | 0.01% | 0.01% | 99.97% | 0.01% | 99.97% | 0.01% |
| DdCBE_ | | | | | | | | | |
| replicate3 | | | | | | | | | |
| ND2- | T | 0.01% | 0.03% | 0.01% | 0.02% | 0.01% | 0.74% | 0.01% | 99.99% |
| DdCBE_ | | | | | | | | | |
| replicate3 | | | | | | | | | |
| ND2- | C | 0.01% | 99.96% | 99.98% | 99.96% | 0.00% | 99.25% | 0.01% | 0.01% |
| DdCBE_ | | | | | | | | | |
| replicate3 | | | | | | | | | |
| ND2- | G | 0.01% | 0.00% | 0.01% | 0.00% | 0.01% | 0.00% | 0.01% | 0.00% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.01% | 0.00% | 0.00% | 0.00% | 0.00% |
| DdCBE_ | | | | | | | | | |
| replicate3 | | | | | | | | | |
| Batch | Nucleotide | G | T | T | C | T | A | C | C |
| ND2- | A | 0.00% | 0.01% | 0.01% | 0.01% | 0.02% | 99.98% | 0.00% | 0.01% |
| DdCBE_ | | | | | | | | | |
| replicate 1 | | | | | | | | | |
| ND2- | T | 0.01% | 99.97% | 99.98% | 0.01% | 99.97% | 0.01% | 0.02% | 0.02% |
| DdCBE_ | | | | | | | | | |
| replicate 1 | | | | | | | | | |
| ND2- | C | 0.01% | 0.01% | 0.01% | 99.98% | 0.01% | 0.00% | 99.97% | 99.96% |
| DdCBE_ | | | | | | | | | |
| replicate 1 | | | | | | | | | |
| ND2- | G | 99.98% | 0.01% | 0.00% | 0.00% | 0.00% | 0.01% | 0.00% | 0.00% |
| DdCBE_ | | | | | | | | | |
| replicate 1 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ND2- | A | 0.01% | 0.01% | 0.00% | 0.00% | 0.01% | 99.99% | 0.00% | 0.01% |
| DdCBE_ | | | | | | | | | |
| replicate2 | | | | | | | | | |
| ND2- | T | 0.00% | 99.97% | 100.00% | 0.01% | 99.98% | 0.00% | 0.01% | 0.02% |
| DdCBE_ | | | | | | | | | |
| replicate2 | | | | | | | | | |
| ND2- | C | 0.00% | 0.01% | 0.00% | 99.99% | 0.01% | 0.00% | 99.98% | 99.96% |
| DdCBE_ | | | | | | | | | |
| replicate2 | | | | | | | | | |
| ND2- | G | 99.99% | 0.01% | 0.00% | 0.00% | 0.00% | 0.01% | 0.00% | 0.00% |
| DdCBE_ | | | | | | | | | |
| replicate2 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND2- | A | 0.01% | 0.01% | 0.01% | 0.00% | 0.01% | 99.96% | 0.00% | 0.01% |
| DdCBE_ | | | | | | | | | |
| replicate3 | | | | | | | | | |
| ND2- | T | 0.01% | 99.97% | 99.97% | 0.01% | 99.97% | 0.01% | 0.02% | 0.02% |
| DdCBE_ | | | | | | | | | |
| replicate3 | | | | | | | | | |
| ND2- | C | 0.00% | 0.02% | 0.02% | 99.98% | 0.01% | 0.00% | 99.98% | 99.96% |
| DdCBE_ | | | | | | | | | |
| replicate3 | | | | | | | | | |
| ND2- | G | 99.98% | 0.00% | 0.00% | 0.01% | 0.00% | 0.03% | 0.00% | 0.01% |
| DdCBE_ | | | | | | | | | |
| replicate3 | | | | | | | | | |
| ND2- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND2- | — | 0.00% | 0.00% | 0.00% | 0.00% | 0.00% | 0.00% | 0.00% | 0.00% |
| DdCBE_ | | | | | | | | | |
| replicate3 | | | | | | | | | |

| Batch | Nucleotide | A | C |
|---|---|---|---|
| ND2- | A | 99.96% | 0.00% |
| DdCBE_ | | | |
| replicate 1 | | | |
| ND2- | T | 0.01% | 0.02% |
| DdCBE_ | | | |
| replicate 1 | | | |
| ND2- | C | 0.01% | 99.98% |
| DdCBE_ | | | |
| replicate 1 | | | |
| ND2- | G | 0.01% | 0.00% |
| DdCBE_ | | | |
| replicate 1 | | | |
| ND2- | N | 0.00% | 0.00% |
| DdCBE_ | | | |
| replicate1 | | | |
| ND2- | — | 0.02% | 0.00% |
| DdCBE_ | | | |
| replicate1 | | | |
| ND2- | A | 99.96% | 0.01% |
| DdCBE_ | | | |
| replicate2 | | | |
| ND2- | T | 0.01% | 0.02% |
| DdCBE_ | | | |
| replicate2 | | | |
| ND2- | C | 0.01% | 99.97% |
| DdCBE_ | | | |
| replicate2 | | | |
| ND2- | G | 0.02% | 0.00% |
| DdCBE_ | | | |
| replicate2 | | | |
| ND2- | N | 0.00% | 0.00% |
| DdCBE_ | | | |
| replicate2 | | | |
| ND2- | — | 0.01% | 0.00% |
| DdCBE_ | | | |
| replicate2 | | | |
| ND2- | A | 99.97% | 0.01% |
| DdCBE_ | | | |
| replicate3 | | | |
| ND2- | T | 0.00% | 0.02% |
| DdCBE_ | | | |
| replicate3 | | | |
| ND2- | C | 0.02% | 99.98% |
| DdCBE_ | | | |
| replicate3 | | | |
| ND2- | G | 0.01% | 0.00% |
| DdCBE_ | | | |
| replicate3 | | | |
| ND2- | N | 0.00% | 0.00% |
| DdCBE_ | | | |
| replicate3 | | | |
| ND2- | — | 0.00% | 0.00% |
| DdCBE_ | | | |
| replicate3 | | | |

TABLE 12G
Base Percentages at TALE Binding Sites-ND1-DdCBE

| Batch | Nucleotide | T | C | C | T | A | T | T | T |
|---|---|---|---|---|---|---|---|---|---|
| ND1- | A | 0.00% | 0.03% | 0.00% | 0.00% | 99.56% | 0.00% | 0.00% | 0.00% |
| DdCBE_ | | | | | | | | | |
| replicate1 | | | | | | | | | |
| ND1- | T | 99.97% | 0.12% | 0.00% | 99.91% | 0.09% | 99.97% | 100.00% | 100.00% |
| DdCBE_ | | | | | | | | | |
| replicate1 | | | | | | | | | |
| ND1- | C | 0.03% | 99.85% | 100.00% | 0.09% | 0.35% | 0.03% | 0.00% | 0.00% |
| DdCBE_ | | | | | | | | | |
| replicate1 | | | | | | | | | |
| ND1- | G | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 0.01% | 0.02% | 0.01% | 0.00% | 99.47% | 0.01% | 0.00% | 0.00% |
| DdCBE_ | | | | | | | | | |
| replicate2 | | | | | | | | | |
| ND1- | T | 99.97% | 0.22% | 0.03% | 99.96% | 0.03% | 99.98% | 99.99% | 99.99% |
| DdCBE_ | | | | | | | | | |
| replicate2 | | | | | | | | | |
| ND1- | C | 0.02% | 99.75% | 99.96% | 0.04% | 0.49% | 0.01% | 0.01% | 0.01% |
| DdCBE_ | | | | | | | | | |
| replicate2 | | | | | | | | | |
| ND1- | G | 0.00% | 0.00% | 0.00% | 0.00% | 0.01% | 0.00% | 0.00% | 0.00% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 0.00% | 0.01% | 0.01% | 0.00% | 99.46% | 0.00% | 0.00% | 0.00% |
| DdCBE_ | | | | | | | | | |
| replicate3 | | | | | | | | | |
| ND1- | T | 99.98% | 0.15% | 0.02% | 99.95% | 0.01% | 99.98% | 99.98% | 99.98% |
| DdCBE_ | | | | | | | | | |
| replicate3 | | | | | | | | | |
| ND1- | C | 0.02% | 99.83% | 99.97% | 0.05% | 0.53% | 0.02% | 0.01% | 0.01% |
| DdCBE_ | | | | | | | | | |
| replicate3 | | | | | | | | | |
| ND1- | G | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | G | C | C | T | A | G | C | C |
| ND1- | A | 0.03% | 0.03% | 0.03% | 0.00% | 99.97% | 0.00% | 0.00% | 0.03% |
| DdCBE_ | | | | | | | | | |
| replicate1 | | | | | | | | | |
| ND1- | T | 0.00% | 0.00% | 0.06% | 99.91% | 0.00% | 0.00% | 0.03% | 0.00% |
| DdCBE_ | | | | | | | | | |
| replicate1 | | | | | | | | | |
| ND1- | C | 0.00% | 99.97% | 99.91% | 0.09% | 0.03% | 0.00% | 99.97% | 99.97% |
| DdCBE_ | | | | | | | | | |
| replicate1 | | | | | | | | | |
| ND1- | G | 99.97% | 0.00% | 0.00% | 0.00% | 0.00% | 100.00% | 0.00% | 0.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 0.01% | 0.03% | 0.00% | 0.01% | 99.92% | 0.01% | 0.01% | 0.00% |
| DdCBE_ | | | | | | | | | |
| replicate2 | | | | | | | | | |
| ND1- | T | 0.01% | 0.01% | 0.01% | 99.94% | 0.02% | 0.01% | 0.01% | 0.02% |
| DdCBE_ | | | | | | | | | |
| replicate2 | | | | | | | | | |
| ND1- | C | 0.00% | 99.95% | 99.98% | 0.05% | 0.06% | 0.01% | 99.98% | 99.97% |
| DdCBE_ | | | | | | | | | |
| replicate2 | | | | | | | | | |
| ND1- | G | 99.98% | 0.00% | 0.00% | 0.00% | 0.01% | 99.97% | 0.00% | 0.00% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 0.01% | 0.03% | 0.00% | 0.00% | 99.89% | 0.01% | 0.01% | 0.00% |
| DdCBE_ | | | | | | | | | |
| replicate3 | | | | | | | | | |
| ND1- | T | 0.00% | 0.00% | 0.00% | 99.95% | 0.01% | 0.01% | 0.01% | 0.01% |
| DdCBE_ | | | | | | | | | |
| replicate3 | | | | | | | | | |
| ND1- | C | 0.00% | 99.96% | 100.00% | 0.05% | 0.08% | 0.00% | 99.98% | 99.99% |
| DdCBE_ | | | | | | | | | |
| replicate3 | | | | | | | | | |
| ND1- | G | 99.99% | 0.00% | 0.00% | 0.00% | 0.01% | 99.98% | 0.00% | 0.00% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | G | A | T | C | A | G | G | G |
| ND1- | A | 5.28% | 99.85% | 0.00% | 0.00% | 99.94% | 0.00% | 0.00% | 0.00% |
| DdCBE_ | | | | | | | | | |
| replicate1 | | | | | | | | | |
| ND1- | T | 0.00% | 0.12% | 100.00% | 0.06% | 0.00% | 0.09% | 0.00% | 0.00% |
| DdCBE_ | | | | | | | | | |
| replicate1 | | | | | | | | | |
| ND1- | C | 0.00% | 0.00% | 0.00% | 99.94% | 0.03% | 0.00% | 0.03% | 0.00% |
| DdCBE_ | | | | | | | | | |
| replicate1 | | | | | | | | | |
| ND1- | G | 94.72% | 0.03% | 0.00% | 0.00% | 0.03% | 99.91% | 99.97% | 100.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 4.93% | 99.88% | 0.00% | 0.02% | 99.97% | 0.02% | 0.01% | 0.01% |
| DdCBE_ | | | | | | | | | |
| replicate2 | | | | | | | | | |
| ND1- | T | 0.02% | 0.10% | 99.98% | 0.03% | 0.01% | 0.05% | 0.03% | 0.00% |
| DdCBE_ | | | | | | | | | |
| replicate2 | | | | | | | | | |
| ND1- | C | 0.05% | 0.01% | 0.01% | 99.94% | 0.01% | 0.00% | 0.00% | 0.00% |
| DdCBE_ | | | | | | | | | |
| replicate2 | | | | | | | | | |
| ND1- | G | 95.00% | 0.01% | 0.00% | 0.00% | 0.01% | 99.93% | 99.96% | 99.98% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 4.41% | 99.92% | 0.01% | 0.01% | 99.98% | 0.01% | 0.02% | 0.00% |
| DdCBE_ | | | | | | | | | |
| replicate3 | | | | | | | | | |
| ND1- | T | 0.05% | 0.07% | 99.98% | 0.02% | 0.00% | 0.07% | 0.02% | 0.01% |
| DdCBE_ | | | | | | | | | |
| replicate3 | | | | | | | | | |
| ND1- | C | 0.03% | 0.00% | 0.01% | 99.97% | 0.01% | 0.00% | 0.00% | 0.00% |
| DdCBE_ | | | | | | | | | |
| replicate3 | | | | | | | | | |
| ND1- | G | 95.51% | 0.01% | 0.00% | 0.00% | 0.01% | 99.91% | 99.96% | 99.99% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | A | C | T | A | C | G | C | C |
| ND1- | A | 99.97% | 0.03% | 0.00% | 100.00% | 0.23% | 0.09% | 0.00% | 0.00% |
| DdCBE_ | | | | | | | | | |
| replicate1 | | | | | | | | | |
| ND1- | T | 0.00% | 0.00% | 100.00% | 0.00% | 0.00% | 0.09% | 0.00% | 0.00% |
| DdCBE_ | | | | | | | | | |
| replicate1 | | | | | | | | | |
| ND1- | C | 0.03% | 99.97% | 0.00% | 0.00% | 99.77% | 0.82% | 100.00% | 100.00% |
| DdCBE_ | | | | | | | | | |
| replicate1 | | | | | | | | | |
| ND1- | G | 0.00% | 0.00% | 0.00% | 0.00% | 0.00% | 99.00% | 0.00% | 0.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 99.97% | 0.01% | 0.01% | 99.98% | 0.14% | 0.03% | 0.01% | 0.01% |
| DdCBE_ | | | | | | | | | |
| replicate2 | | | | | | | | | |
| ND1- | T | 0.00% | 0.01% | 99.98% | 0.01% | 0.01% | 0.07% | 0.01% | 0.01% |
| DdCBE_ | | | | | | | | | |
| replicate2 | | | | | | | | | |
| ND1- | C | 0.01% | 99.98% | 0.01% | 0.01% | 99.86% | 1.08% | 99.98% | 99.98% |
| DdCBE_ | | | | | | | | | |
| replicate2 | | | | | | | | | |
| ND1- | G | 0.01% | 0.00% | 0.00% | 0.00% | 0.00% | 98.83% | 0.00% | 0.00% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 99.99% | 0.01% | 0.01% | 99.96% | 0.15% | 0.02% | 0.01% | 0.00% |
| DdCBE_ | | | | | | | | | |
| replicate3 | | | | | | | | | |
| ND1- | T | 0.00% | 0.00% | 99.95% | 0.01% | 0.00% | 0.10% | 0.00% | 0.01% |
| DdCBE_ | | | | | | | | | |
| replicate3 | | | | | | | | | |
| ND1- | C | 0.00% | 99.99% | 0.03% | 0.02% | 99.85% | 0.99% | 99.98% | 99.99% |
| DdCBE_ | | | | | | | | | |
| replicate3 | | | | | | | | | |
| ND1- | G | 0.00% | 0.00% | 0.00% | 0.01% | 0.00% | 98.89% | 0.00% | 0.00% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | A | T | T | C | T | A | G | C |
| ND1- | A | 99.85% | 0.00% | 0.00% | 0.00% | 0.00% | 99.88% | 0.03% | 0.06% |
| DdCBE_ | | | | | | | | | |
| replicate1 | | | | | | | | | |
| ND1- | T | 0.06% | 99.97% | 100.00% | 0.06% | 99.97% | 0.06% | 0.06% | 0.03% |
| DdCBE_ | | | | | | | | | |
| replicate1 | | | | | | | | | |
| ND1- | C | 0.09% | 0.03% | 0.00% | 99.94% | 0.03% | 0.00% | 0.00% | 99.91% |
| DdCBE_ | | | | | | | | | |
| replicate1 | | | | | | | | | |
| ND1- | G | 0.00% | 0.00% | 0.00% | 0.00% | 0.00% | 0.06% | 99.91% | 0.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 99.84% | 0.00% | 0.00% | 0.01% | 0.00% | 99.89% | 0.01% | 0.02% |
| DdCBE_ | | | | | | | | | |
| replicate2 | | | | | | | | | |
| ND1- | T | 0.06% | 99.98% | 99.99% | 0.03% | 99.97% | 0.06% | 0.01% | 0.04% |
| DdCBE_ | | | | | | | | | |
| replicate2 | | | | | | | | | |
| ND1- | C | 0.08% | 0.02% | 0.01% | 99.95% | 0.02% | 0.04% | 0.00% | 99.94% |
| DdCBE_ | | | | | | | | | |
| replicate2 | | | | | | | | | |
| ND1- | G | 0.01% | 0.00% | 0.00% | 0.00% | 0.00% | 0.00% | 99.97% | 0.00% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 99.88% | 0.00% | 0.00% | 0.00% | 0.00% | 99.91% | 0.02% | 0.00% |
| DdCBE_ | | | | | | | | | |
| replicate3 | | | | | | | | | |
| ND1- | T | 0.02% | 99.98% | 99.99% | 0.03% | 99.99% | 0.04% | 0.01% | 0.02% |
| DdCBE_ | | | | | | | | | |
| replicate3 | | | | | | | | | |
| ND1- | C | 0.10% | 0.01% | 0.01% | 99.96% | 0.01% | 0.05% | 0.01% | 99.97% |
| DdCBE_ | | | | | | | | | |
| replicate3 | | | | | | | | | |
| ND1- | G | 0.00% | 0.01% | 0.00% | 0.00% | 0.00% | 0.00% | 99.96% | 0.00% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | G | T | T | T | A | C | T | C |
| ND1- | A | 0.00% | 0.06% | 0.00% | 0.00% | 99.85% | 0.06% | 0.00% | 0.03% |
| DdCBE_ | | | | | | | | | |
| replicate1 | | | | | | | | | |
| ND1- | T | 0.00% | 99.82% | 99.94% | 100.00% | 0.06% | 0.03% | 99.97% | 0.29% |
| DdCBE_ | | | | | | | | | |
| replicate1 | | | | | | | | | |
| ND1- | C | 0.00% | 0.12% | 0.06% | 0.00% | 0.03% | 99.91% | 0.03% | 99.65% |
| DdCBE_ | | | | | | | | | |
| replicate1 | | | | | | | | | |
| ND1- | G | 100.00% | 0.00% | 0.00% | 0.00% | 0.06% | 0.00% | 0.00% | 0.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.03% |
| DdCBE_ | | | | | | | | | |
| replicate1 | | | | | | | | | |
| ND1- | A | 0.02% | 0.03% | 0.00% | 0.00% | 99.92% | 0.03% | 0.00% | 0.01% |
| DdCBE_ | | | | | | | | | |
| replicate2 | | | | | | | | | |
| ND1- | T | 0.01% | 99.82% | 99.96% | 99.98% | 0.03% | 0.01% | 99.96% | 0.30% |
| DdCBE_ | | | | | | | | | |
| replicate2 | | | | | | | | | |
| ND1- | C | 0.00% | 0.09% | 0.03% | 0.02% | 0.04% | 99.95% | 0.04% | 99.68% |
| DdCBE_ | | | | | | | | | |
| replicate2 | | | | | | | | | |
| ND1- | G | 99.97% | 0.07% | 0.00% | 0.00% | 0.01% | 0.00% | 0.00% | 0.00% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.01% | 0.00% | 0.00% | 0.00% | 0.00% |
| DdCBE_ | | | | | | | | | |
| replicate2 | | | | | | | | | |
| ND1- | A | 0.01% | 0.03% | 0.00% | 0.00% | 99.93% | 0.02% | 0.00% | 0.01% |
| DdCBE_ | | | | | | | | | |
| replicate3 | | | | | | | | | |
| ND1- | T | 0.01% | 99.79% | 99.97% | 99.97% | 0.03% | 0.02% | 99.97% | 0.33% |
| DdCBE_ | | | | | | | | | |
| replicate3 | | | | | | | | | |
| ND1- | C | 0.00% | 0.13% | 0.03% | 0.02% | 0.03% | 99.96% | 0.03% | 99.65% |
| DdCBE_ | | | | | | | | | |
| replicate3 | | | | | | | | | |
| ND1- | G | 99.97% | 0.05% | 0.00% | 0.00% | 0.01% | 0.00% | 0.00% | 0.00% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | T | G | A | G | C | A | T | C |
| ND1- | A | 0.15% | 0.00% | 99.97% | 0.00% | 0.00% | 99.97% | 0.00% | 0.03% |
| DdCBE_ | | | | | | | | | |
| replicate1 | | | | | | | | | |
| ND1- | T | 98.74% | 0.00% | 0.00% | 0.00% | 0.00% | 0.00% | 100.00 | 0.00% |
| DdCBE_ | | | | | | | | | |
| replicate1 | | | | | | | | | |
| ND1- | C | 1.03% | 0.00% | 0.00% | 0.03% | 100.00 | 0.00% | 0.00% | 99.97% |
| DdCBE_ | | | | | | | | | |
| replicate1 | | | | | | | | | |
| ND1- | G | 0.09% | 100.00% | 0.03% | 99.97% | 0.00% | 0.03% | 0.00% | 0.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 0.11% | 0.02% | 99.91% | 0.01% | 0.01% | 99.92% | 0.02% | 0.00% |
| DdCBE_ | | | | | | | | | |
| replicate2 | | | | | | | | | |
| ND1- | T | 98.81% | 0.02% | 0.02% | 0.01% | 0.03% | 0.04% | 99.96 | 0.02% |
| DdCBE_ | | | | | | | | | |
| replicate2 | | | | | | | | | |
| ND1- | C | 0.96% | 0.00% | 0.01% | 0.00% | 99.96% | 0.03% | 0.02% | 99.98% |
| DdCBE_ | | | | | | | | | |
| replicate2 | | | | | | | | | |
| ND1- | G | 0.12% | 99.96% | 0.06% | 99.99% | 0.01% | 0.01% | 0.00% | 0.00% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 0.10% | 0.02% | 99.93% | 0.01% | 0.00% | 99.92% | 0.01% | 0.02% |
| DdCBE_ | | | | | | | | | |
| replicate3 | | | | | | | | | |
| ND1- | T | 98.81% | 0.00% | 0.02% | 0.00% | 0.03% | 0.03% | 99.97% | 0.02% |
| DdCBE_ | | | | | | | | | |
| replicate3 | | | | | | | | | |
| ND1- | C | 0.95% | 0.00% | 0.02% | 0.00% | 99.96% | 0.04% | 0.02% | 99.96% |
| DdCBE_ | | | | | | | | | |
| replicate3 | | | | | | | | | |
| ND1- | G | 0.14% | 99.98% | 0.04% | 99.99% | 0.01% | 0.01% | 0.00% | 0.00% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | C | T | G | A | T | C | G | G |
| ND1- | A | 0.00% | 0.00% | 0.03% | 99.85% | 0.00% | 0.00% | 0.03% | 0.03% |
| DdCBE_ | | | | | | | | | |
| replicate1 | | | | | | | | | |
| ND1- | T | 0.00% | 99.97% | 0.00% | 0.00% | 99.79% | 0.38% | 0.00% | 0.03% |
| DdCBE_ | | | | | | | | | |
| replicate1 | | | | | | | | | |
| ND1- | C | 100.00% | 0.03% | 0.03% | 0.15% | 0.21% | 99.62% | 0.00% | 0.23% |
| DdCBE_ | | | | | | | | | |
| replicate1 | | | | | | | | | |
| ND1- | G | 0.00% | 0.00% | 99.94% | 0.00% | 0.00% | 0.00% | 99.97% | 99.71% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 0.01% | 0.01% | 0.10% | 99.89% | 0.01% | 0.01% | 0.02% | 0.02% |
| DdCBE_ | | | | | | | | | |
| replicate2 | | | | | | | | | |
| ND1- | T | 0.00% | 99.88% | 0.01% | 0.01% | 99.83% | 0.59% | 0.03% | 0.02% |
| DdCBE_ | | | | | | | | | |
| replicate2 | | | | | | | | | |
| ND1- | C | 99.98% | 0.10% | 0.05% | 0.08% | 0.16% | 99.40% | 0.03% | 0.20% |
| DdCBE_ | | | | | | | | | |
| replicate2 | | | | | | | | | |
| ND1- | G | 0.00% | 0.00% | 99.84% | 0.02% | 0.00% | 0.00% | 99.92% | 99.76% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 0.01% | 0.00% | 0.11% | 99.90% | 0.03% | 0.00% | 0.03% | 0.02% |
| DdCBE_ | | | | | | | | | |
| replicate3 | | | | | | | | | |
| ND1- | T | 0.01% | 99.84% | 0.00% | 0.01% | 99.84% | 0.46% | 0.04% | 0.03% |
| DdCBE_ | | | | | | | | | |
| replicate3 | | | | | | | | | |
| ND1- | C | 99.97% | 0.15% | 0.04% | 0.09% | 0.13% | 99.53% | 0.02% | 0.19% |
| DdCBE_ | | | | | | | | | |
| replicate3 | | | | | | | | | |
| ND1- | G | 0.00% | 0.00% | 99.85% | 0.00% | 0.00% | 0.00% | 99.91% | 99.76% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.01% | 0.00% | 0.00% | 0.00% | 0.00% | 0.00% | 0.00% |
| DdCBE_ | | | | | | | | | |
| replicate3 | | | | | | | | | |
| Batch | Nucleotide | C | A | C | C | T | C | T | A |
| ND1- | A | 0.03% | 99.88% | 0.00% | 0.00% | 0.00% | 0.00% | 0.00% | 99.88% |
| DdCBE_ | | | | | | | | | |
| replicate1 | | | | | | | | | |
| ND1- | T | 0.00% | 0.00% | 0.00% | 0.00% | 99.97% | 0.03% | 99.97% | 0.09% |
| DdCBE_ | | | | | | | | | |
| replicate1 | | | | | | | | | |
| ND1- | C | 99.97% | 0.09% | 100.00% | 100.00% | 0.03% | 99.97% | 0.03% | 0.00% |
| DdCBE_ | | | | | | | | | |
| replicate1 | | | | | | | | | |
| ND1- | G | 0.00% | 0.03% | 0.00% | 0.00% | 0.00% | 0.00% | 0.00% | 0.03% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 0.01% | 99.94% | 0.01% | 0.01% | 0.01% | 0.01% | 0.00% | 99.86% |
| DdCBE_ | | | | | | | | | |
| replicate2 | | | | | | | | | |
| ND1- | T | 0.03% | 0.01% | 0.01% | 0.01% | 99.98% | 0.05% | 99.98% | 0.04% |
| DdCBE_ | | | | | | | | | |
| replicate2 | | | | | | | | | |
| ND1- | C | 99.96% | 0.04% | 99.99% | 99.98% | 0.01% | 99.94% | 0.02% | 0.09% |
| DdCBE_ | | | | | | | | | |
| replicate2 | | | | | | | | | |
| ND1- | G | 0.00% | 0.01% | 0.00% | 0.00% | 0.00% | 0.00% | 0.00% | 0.01% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 0.00% | 99.90% | 0.00% | 0.01% | 0.01% | 0.00% | 0.01% | 99.81% |
| DdCBE_ | | | | | | | | | |
| replicate3 | | | | | | | | | |
| ND1- | T | 0.04% | 0.02% | 0.01% | 0.00% | 99.98% | 0.03% | 99.97% | 0.04% |
| DdCBE_ | | | | | | | | | |
| replicate3 | | | | | | | | | |
| ND1- | C | 99.96% | 0.08% | 99.98% | 99.99% | 0.01% | 99.96% | 0.02% | 0.13% |
| DdCBE_ | | | | | | | | | |
| replicate3 | | | | | | | | | |
| ND1- | G | 0.00% | 0.00% | 0.00% | 0.00% | 0.00% | 0.00% | 0.00% | 0.02% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | A | A | T | C | C | T | C | T |
| ND1- | A | 99.82% | 99.79% | 0.00% | 0.03% | 0.00% | 0.00% | 0.00% | 0.03% |
| DdCBE_ | | | | | | | | | |
| replicate1 | | | | | | | | | |
| ND1- | T | 0.03% | 0.00% | 100.00% | 47.25% | 46.54% | 100.00% | 31.24% | 99.91% |
| DdCBE_ | | | | | | | | | |
| replicate1 | | | | | | | | | |
| ND1- | C | 0.09% | 0.21% | 0.00% | 52.73% | 53.46% | 0.00% | 68.76% | 0.06% |
| DdCBE_ | | | | | | | | | |
| replicate1 | | | | | | | | | |
| ND1- | G | 0.06% | 0.00% | 0.00% | 0.00% | 0.00% | 0.00% | 0.00% | 0.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 99.85% | 99.83% | 0.01% | 0.01% | 0.01% | 0.00% | 0.00% | 0.01% |
| DdCBE_ | | | | | | | | | |
| replicate2 | | | | | | | | | |
| ND1- | T | 0.05% | 0.02% | 99.98% | 47.68% | 46.95% | 99.99% | 29.89% | 99.97% |
| DdCBE_ | | | | | | | | | |
| replicate2 | | | | | | | | | |
| ND1- | C | 0.08% | 0.15% | 0.01% | 52.31% | 53.04% | 0.01% | 70.11% | 0.02% |
| DdCBE_ | | | | | | | | | |
| replicate2 | | | | | | | | | |
| ND1- | G | 0.02% | 0.00% | 0.00% | 0.00% | 0.00% | 0.00% | 0.00% | 0.00% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.01% | 0.00% | 0.00% | 0.00% |
| DdCBE_ | | | | | | | | | |
| replicate2 | | | | | | | | | |
| ND1- | A | 99.81% | 99.85% | 0.01% | 0.00% | 0.00% | 0.00% | 0.00% | 0.01% |
| DdCBE_ | | | | | | | | | |
| replicate3 | | | | | | | | | |
| ND1- | T | 0.05% | 0.01% | 99.98% | 43.02% | 42.41% | 99.97% | 27.64% | 99.97% |
| DdCBE_ | | | | | | | | | |
| replicate3 | | | | | | | | | |
| ND1- | C | 0.11% | 0.15% | 0.01% | 56.98% | 57.58% | 0.03% | 72.36% | 0.02% |
| DdCBE_ | | | | | | | | | |
| replicate3 | | | | | | | | | |
| ND1- | G | 0.02% | 0.00% | 0.00% | 0.00% | 0.00% | 0.00% | 0.00% | 0.00% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.01% | 0.00% | 0.00% | 0.00% |
| DdCBE_ | | | | | | | | | |
| replicate3 | | | | | | | | | |
| Batch | Nucleotide | A | A | A | C | T | C | A | A |
| ND1- | A | 99.97% | 100.00% | 99.97% | 0.15% | 0.00% | 0.12% | 99.97% | 100.00% |
| DdCBE_ | | | | | | | | | |
| replicate1 | | | | | | | | | |
| ND1- | T | 0.00% | 0.00% | 0.00% | 0.00% | 99.97% | 0.00% | 0.00% | 0.00% |
| DdCBE_ | | | | | | | | | |
| replicate1 | | | | | | | | | |
| ND1- | C | 0.03% | 0.00% | 0.03% | 99.85% | 0.03% | 99.88% | 0.03% | 0.00% |
| DdCBE_ | | | | | | | | | |
| replicate1 | | | | | | | | | |
| ND1- | G | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ND1- | A | 99.95% | 99.97% | 99.97% | 0.03% | 0.01% | 0.06% | 99.97% | 99.95% |
| DdCBE_ | | | | | | | | | |
| replicate2 | | | | | | | | | |
| ND1- | T | 0.01% | 0.00% | 0.01% | 0.01% | 99.96% | 0.02% | 0.01% | 0.01% |
| DdCBE_ | | | | | | | | | |
| replicate2 | | | | | | | | | |
| ND1- | C | 0.03% | 0.02% | 0.02% | 99.95% | 0.03% | 99.92% | 0.02% | 0.03% |
| DdCBE_ | | | | | | | | | |
| replicate2 | | | | | | | | | |
| ND1- | G | 0.01% | 0.01% | 0.00% | 0.00% | 0.00% | 0.00% | 0.01% | 0.01% |
| DdCBE_ | | | | | | | | | |
| replicate2 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ND1- | A | 99.93% | 99.96% | 99.97% | 0.08% | 0.00% | 0.06% | 99.97% | 99.96% |
| DdCBE_ | | | | | | | | | |
| replicate3 | | | | | | | | | |
| ND1- | T | 0.00% | 0.00% | 0.01% | 0.00% | 99.97% | 0.01% | 0.01% | 0.01% |
| DdCBE_ | | | | | | | | | |
| replicate3 | | | | | | | | | |
| ND1- | C | 0.05% | 0.03% | 0.01% | 99.92% | 0.02% | 99.93% | 0.01% | 0.03% |
| DdCBE_ | | | | | | | | | |
| replicate3 | | | | | | | | | |
| ND1- | G | 0.02% | 0.01% | 0.01% | 0.00% | 0.00% | 0.00% | 0.01% | 0.01% |
| DdCBE_ | | | | | | | | | |
| replicate3 | | | | | | | | | |
| ND1- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ND1- | — | 0.00% | 0.00% | 0.00% | 0.00% | 0.00% | 0.00% | 0.00% | 0.00% |
| DdCBE_ | | | | | | | | | |
| replicate3 | | | | | | | | | |

| Batch | Nucleotide | C | G | C |
|---|---|---|---|---|
| ND1- | A | 0.00% | 0.06% | 0.03% |
| DdCBE_ | | | | |
| replicate1 | | | | |
| ND1- | T | 0.03% | 0.00% | 0.00% |
| DdCBE_ | | | | |
| replicate1 | | | | |
| ND1- | C | 99.94% | 0.03% | 99.97% |
| DdCBE_ | | | | |
| replicate1 | | | | |
| ND1- | G | 0.03% | 99.91% | 0.00% |
| DdCBE_ | | | | |
| replicate1 | | | | |
| ND1- | N | 0.00% | 0.00% | 0.00% |
| DdCBE_ | | | | |
| replicate1 | | | | |
| ND1- | — | 0.00% | 0.00% | 0.00% |
| DdCBE_ | | | | |
| replicate1 | | | | |
| ND1- | A | 0.01% | 0.01% | 0.01% |
| DdCBE_ | | | | |
| replicate2 | | | | |
| ND1- | T | 0.03% | 0.04% | 0.00% |
| DdCBE_ | | | | |
| replicate2 | | | | |
| ND1- | C | 99.97% | 0.01% | 99.98% |
| DdCBE_ | | | | |
| replicate2 | | | | |
| ND1- | G | 0.00% | 99.94% | 0.00% |
| DdCBE_ | | | | |
| replicate2 | | | | |
| ND1- | N | 0.00% | 0.00% | 0.00% |
| DdCBE_ | | | | |
| replicate2 | | | | |
| ND1- | — | 0.00% | 0.00% | 0.00% |
| DdCBE_ | | | | |
| replicate2 | | | | |
| ND1- | A | 0.00% | 0.00% | 0.02% |
| DdCBE_ | | | | |
| replicate3 | | | | |
| ND1- | T | 0.03% | 0.03% | 0.00% |
| DdCBE_ | | | | |
| replicate3 | | | | |
| ND1- | C | 99.97% | 0.01% | 99.98% |
| DdCBE_ | | | | |
| replicate3 | | | | |
| ND1- | G | 0.00% | 99.96% | 0.00% |
| DdCBE_ | | | | |
| replicate3 | | | | |
| ND1- | N | 0.00% | 0.00% | 0.00% |
| DdCBE_ | | | | |
| replicate3 | | | | |
| ND1- | — | 0.00% | 0.00% | 0.00% |
| DdCBE_ | | | | |
| replicate3 | | | | |

TABLE 12H
Base Percentages at TALE Binding Sites-ATP8-DdCBE

| Batch | Nucleotide | C | C | T | C | A | T | C | A |
|---|---|---|---|---|---|---|---|---|---|
| ATP8- | A | 0.07% | 0.03% | 0.01% | 0.01% | 99.97% | 0.02% | 0.02% | 99.96% |
| DdCBE_ | | | | | | | | | |
| replicate 1 | | | | | | | | | |
| ATP8- | T | 0.06% | 0.03% | 99.96% | 0.18% | 0.01% | 99.97% | 0.04% | 0.03% |
| DdCBE_ | | | | | | | | | |
| replicate 1 | | | | | | | | | |
| ATP8- | C | 99.86% | 99.93% | 0.03% | 99.81% | 0.01% | 0.02% | 99.93% | 0.01% |
| DdCBE_ | | | | | | | | | |
| replicate1 | | | | | | | | | |
| ATP8 | G | 0.01% | 0.00% | 0.00% | 0.00% | 0.01% | 0.00% | 0.00% | 0.00% |
| DdCBE_ | | | | | | | | | |
| replicate 1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ATP8- | A | 0.07% | 0.02% | 0.01% | 0.04% | 99.97% | 0.01% | 0.03% | 99.95% |
| DdCBE_ | | | | | | | | | |
| replicate2 | | | | | | | | | |
| ATP8- | T | 0.06% | 0.02% | 99.96% | 0.23% | 0.01% | 99.99% | 0.07% | 0.02% |
| DdCBE_ | | | | | | | | | |
| replicate2 | | | | | | | | | |
| ATP8- | C | 99.87% | 99.95% | 0.03% | 99.73% | 0.01% | 0.00% | 99.90% | 0.02% |
| DdCBE_ | | | | | | | | | |
| replicate2 | | | | | | | | | |
| ATP8- | G | 0.01% | 0.00% | 0.00% | 0.00% | 0.01% | 0.00% | 0.00% | 0.01% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.00% | 0.01% | 0.00% | 0.00% | 0.00% | 0.00% | 0.00% | 0.00% |
| DdCBE_ | | | | | | | | | |
| replicate2 | | | | | | | | | |
| ATP8- | A | 0.06% | 0.03% | 0.02% | 0.01% | 99.96% | 0.03% | 0.02% | 99.93% |
| DdCBE_ | | | | | | | | | |
| replicate3 | | | | | | | | | |
| ATP8- | T | 0.04% | 0.02% | 99.96% | 0.18% | 0.01% | 99.97% | 0.07% | 0.03% |
| DdCBE_ | | | | | | | | | |
| replicate3 | | | | | | | | | |
| ATP8- | C | 99.90% | 99.94% | 0.02% | 99.80% | 0.02% | 0.00% | 99.92% | 0.03% |
| DdCBE_ | | | | | | | | | |
| replicate3 | | | | | | | | | |
| ATP8- | G | 0.01% | 0.00% | 0.00% | 0.00% | 0.01% | 0.00% | 0.00% | 0.01% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.00% | 0.00% | 0.00% | 0.01% | 0.00% | 0.00% | 0.00% | 0.01% |
| DdCBE_ | | | | | | | | | |
| replicate3 | | | | | | | | | |
| Batch | Nucleotide | A | A | A | C | A | C | A | A |
| ATP8- | A | 99.97% | 99.98% | 99.95% | 0.01% | 99.97% | 0.01% | 99.98% | 99.96% |
| DdCBE_ | | | | | | | | | |
| replicate 1 | | | | | | | | | |
| ATP8- | T | 0.01% | 0.01% | 0.01% | 0.05% | 0.01% | 0.03% | 0.00% | 0.02% |
| DdCBE_ | | | | | | | | | |
| replicate1 | | | | | | | | | |
| ATP8- | C | 0.01% | 0.00% | 0.01% | 99.94% | 0.00% | 99.96% | 0.01% | 0.01% |
| DdCBE_ | | | | | | | | | |
| replicate 1 | | | | | | | | | |
| ATP8- | G | 0.01% | 0.01% | 0.01% | 0.00% | 0.02% | 0.00% | 0.01% | 0.01% |
| DdCBE_ | | | | | | | | | |
| replicate 1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.00% | 0.01% | 0.00% | 0.00% | 0.00% | 0.00% | 0.00% |
| DdCBE_ | | | | | | | | | |
| replicate 1 | | | | | | | | | |
| ATP8- | A | 99.95% | 99.96% | 99.95% | 0.02% | 99.97% | 0.03% | 99.97% | 99.95% |
| DdCBE_ | | | | | | | | | |
| replicate2 | | | | | | | | | |
| ATP8- | T | 0.01% | 0.03% | 0.02% | 0.06% | 0.01% | 0.02% | 0.00% | 0.02% |
| DdCBE_ | | | | | | | | | |
| replicate2 | | | | | | | | | |
| ATP8- | C | 0.02% | 0.00% | 0.01% | 99.91% | 0.00% | 99.95% | 0.01% | 0.02% |
| DdCBE_ | | | | | | | | | |
| replicate2 | | | | | | | | | |
| ATP8- | G | 0.02% | 0.00% | 0.01% | 0.00% | 0.01% | 0.00% | 0.01% | 0.01% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.00% | 0.00% | 0.01% | 0.00% | 0.00% | 0.00% | 0.00% | 0.00% |
| DdCBE_ | | | | | | | | | |
| replicate2 | | | | | | | | | |
| ATP8- | A | 99.95% | 99.96% | 99.95% | 0.02% | 99.96% | 0.01% | 99.98% | 99.93% |
| DdCBE_ | | | | | | | | | |
| replicate3 | | | | | | | | | |
| ATP8- | T | 0.02% | 0.02% | 0.02% | 0.04% | 0.02% | 0.02% | 0.00% | 0.02% |
| DdCBE_ | | | | | | | | | |
| replicate3 | | | | | | | | | |
| ATP8- | C | 0.01% | 0.01% | 0.01% | 99.94% | 0.01% | 99.96% | 0.01% | 0.03% |
| DdCBE_ | | | | | | | | | |
| replicate3 | | | | | | | | | |
| ATP8- | G | 0.02% | 0.02% | 0.00% | 0.00% | 0.01% | 0.00% | 0.01% | 0.01% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.00% | 0.00% | 0.01% | 0.00% | 0.00% | 0.00% | 0.00% | 0.00% |
| DdCBE_ | | | | | | | | | |
| replicate3 | | | | | | | | | |
| Batch | Nucleotide | C | T | C | A | C | C | A | A |
| ATP8- | A | 0.04% | 0.03% | 0.05% | 99.98% | 0.02% | 0.02% | 99.97% | 99.98% |
| DdCBE_ | | | | | | | | | |
| replicate 1 | | | | | | | | | |
| ATP8- | T | 0.02% | 99.95% | 0.07% | 0.01% | 0.01% | 0.01% | 0.01% | 0.01% |
| DdCBE_ | | | | | | | | | |
| replicate 1 | | | | | | | | | |
| ATP8- | C | 99.94% | 0.02% | 99.87% | 0.00% | 99.97% | 99.96% | 0.01% | 0.00% |
| DdCBE_ | | | | | | | | | |
| replicate 1 | | | | | | | | | |
| ATP8- | G | 0.00% | 0.00% | 0.00% | 0.01% | 0.00% | 0.01% | 0.01% | 0.01% |
| DdCBE_ | | | | | | | | | |
| replicate1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | A | 0.01% | 0.01% | 0.05% | 99.97% | 0.01% | 0.03% | 99.97% | 99.96% |
| DdCBE_ | | | | | | | | | |
| replicate2 | | | | | | | | | |
| ATP8- | T | 0.01% | 99.97% | 0.07% | 0.01% | 0.02% | 0.02% | 0.01% | 0.03% |
| DdCBE_ | | | | | | | | | |
| replicate2 | | | | | | | | | |
| ATP8- | C | 99.97% | 0.02% | 99.87% | 0.01% | 99.97% | 99.95% | 0.01% | 0.00% |
| DdCBE_ | | | | | | | | | |
| replicate2 | | | | | | | | | |
| ATP8- | G | 0.00% | 0.00% | 0.00% | 0.00% | 0.00% | 0.00% | 0.00% | 0.01% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.01% | 0.00% | 0.00% | 0.00% | 0.00% | 0.00% | 0.00% | 0.00% |
| DdCBE_ | | | | | | | | | |
| replicate2 | | | | | | | | | |
| ATP8- | A | 0.02% | 0.02% | 0.04% | 99.96% | 0.02% | 0.02% | 99.98% | 99.96% |
| DdCBE_ | | | | | | | | | |
| replicate3 | | | | | | | | | |
| ATP8- | T | 0.02% | 99.96% | 0.04% | 0.01% | 0.01% | 0.02% | 0.01% | 0.02% |
| DdCBE_ | | | | | | | | | |
| replicate3 | | | | | | | | | |
| ATP8- | C | 99.94% | 0.01% | 99.92% | 0.01% | 99.97% | 99.96% | 0.01% | 0.01% |
| DdCBE_ | | | | | | | | | |
| replicate3 | | | | | | | | | |
| ATP8- | G | 0.00% | 0.00% | 0.00% | 0.02% | 0.00% | 0.00% | 0.01% | 0.01% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.02% | 0.00% | 0.00% | 0.01% | 0.00% | 0.00% | 0.00% | 0.00% |
| DdCBE_ | | | | | | | | | |
| replicate3 | | | | | | | | | |
| Batch | Nucleotide | A | A | A | T | T | A | T | A |
| ATP8- | A | 99.99% | 99.97% | 99.93% | 0.01% | 0.14% | 99.98% | 0.04% | 99.98% |
| DdCBE_ | | | | | | | | | |
| replicate1 | | | | | | | | | |
| ATP8- | T | 0.00% | 0.02% | 0.02% | 99.95% | 99.84% | 0.01% | 99.94% | 0.01% |
| DdCBE_ | | | | | | | | | |
| replicate 1 | | | | | | | | | |
| ATP8- | C | 0.00% | 0.00% | 0.00% | 0.03% | 0.01% | 0.00% | 0.01% | 0.00% |
| DdCBE_ | | | | | | | | | |
| replicate 1 | | | | | | | | | |
| ATP8- | G | 0.01% | 0.01% | 0.00% | 0.00% | 0.01% | 0.00% | 0.00% | 0.01% |
| DdCBE_ | | | | | | | | | |
| replicate 1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate 1 | | | | | | | | | |
| ATP8- | — | 0.00% | 0.00% | 0.05% | 0.00% | 0.01% | 0.00% | 0.01% | 0.00% |
| DdCBE_ | | | | | | | | | |
| replicate1 | | | | | | | | | |
| ATP8- | A | 99.99% | 99.97% | 99.94% | 0.02% | 0.16% | 99.97% | 0.04% | 99.96% |
| DdCBE_ | | | | | | | | | |
| replicate2 | | | | | | | | | |
| ATP8- | T | 0.00% | 0.01% | 0.02% | 99.93% | 99.83% | 0.01% | 99.94% | 0.03% |
| DdCBE_ | | | | | | | | | |
| replicate2 | | | | | | | | | |
| ATP8- | C | 0.00% | 0.01% | 0.00% | 0.05% | 0.00% | 0.00% | 0.01% | 0.00% |
| DdCBE_ | | | | | | | | | |
| replicate2 | | | | | | | | | |
| ATP8- | G | 0.01% | 0.01% | 0.01% | 0.00% | 0.00% | 0.01% | 0.00% | 0.01% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.00% | 0.00% | 0.03% | 0.00% | 0.00% | 0.00% | 0.00% | 0.00% |
| DdCBE_ | | | | | | | | | |
| replicate2 | | | | | | | | | |
| ATP8- | A | 99.98% | 99.98% | 99.93% | 0.01% | 0.13% | 99.97% | 0.02% | 99.98% |
| DdCBE_ | | | | | | | | | |
| replicate3 | | | | | | | | | |
| ATP8- | T | 0.00% | 0.01% | 0.03% | 99.94% | 99.85% | 0.02% | 99.96% | 0.01% |
| DdCBE_ | | | | | | | | | |
| replicate3 | | | | | | | | | |
| ATP8- | C | 0.01% | 0.00% | 0.00% | 0.04% | 0.02% | 0.00% | 0.01% | 0.01% |
| DdCBE_ | | | | | | | | | |
| replicate3 | | | | | | | | | |
| ATP8- | G | 0.01% | 0.00% | 0.00% | 0.00% | 0.00% | 0.01% | 0.00% | 0.01% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.00% | 0.00% | 0.04% | 0.00% | 0.00% | 0.00% | 0.00% | 0.00% |
| DdCBE_ | | | | | | | | | |
| replicate3 | | | | | | | | | |
| Batch | Nucleotide | C | C | C | A | A | C | T | A |
| ATP8- | A | 0.01% | 0.01% | 0.03% | 99.98% | 99.25% | 0.01% | 0.02% | 99.97% |
| DdCBE_ | | | | | | | | | |
| replicate 1 | | | | | | | | | |
| ATP8- | T | 0.01% | 0.01% | 0.02% | 0.01% | 0.01% | 0.01% | 99.97% | 0.02% |
| DdCBE_ | | | | | | | | | |
| replicate 1 | | | | | | | | | |
| ATP8- | C | 99.97% | 99.97% | 99.93% | 0.01% | 0.72% | 99.97% | 0.01% | 0.00% |
| DdCBE_ | | | | | | | | | |
| replicate1 | | | | | | | | | |
| ATP8 | G | 0.00% | 0.00% | 0.00% | 0.00% | 0.02% | 0.00% | 0.00% | 0.01% |
| DdCBE_ | | | | | | | | | |
| replicate 1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.00% | 0.01% | 0.00% | 0.00% | 0.00% | 0.00% | 0.00% |
| DdCBE_ | | | | | | | | | |
| replicate 1 | | | | | | | | | |
| ATP8- | A | 0.02% | 0.01% | 0.02% | 99.97% | 99.30% | 0.03% | 0.01% | 99.96% |
| DdCBE_ | | | | | | | | | |
| replicate2 | | | | | | | | | |
| ATP8- | T | 0.03% | 0.03% | 0.02% | 0.01% | 0.02% | 0.00% | 99.97% | 0.02% |
| DdCBE_ | | | | | | | | | |
| replicate2 | | | | | | | | | |
| ATP8- | C | 99.95% | 99.96% | 99.95% | 0.01% | 0.64% | 99.97% | 0.01% | 0.01% |
| DdCBE_ | | | | | | | | | |
| replicate2 | | | | | | | | | |
| ATP8- | G | 0.00% | 0.00% | 0.00% | 0.01% | 0.03% | 0.00% | 0.00% | 0.01% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | A | 0.03% | 0.02% | 0.03% | 99.98% | 99.25% | 0.01% | 0.02% | 99.97% |
| DdCBE_ | | | | | | | | | |
| replicate3 | | | | | | | | | |
| ATP8- | T | 0.00% | 0.02% | 0.03% | 0.00% | 0.02% | 0.02% | 99.97% | 0.01% |
| DdCBE_ | | | | | | | | | |
| replicate3 | | | | | | | | | |
| ATP8- | C | 99.96% | 99.95% | 99.94% | 0.01% | 0.72% | 99.95% | 0.01% | 0.02% |
| DdCBE_ | | | | | | | | | |
| replicate3 | | | | | | | | | |
| ATP8- | G | 0.00% | 0.00% | 0.00% | 0.01% | 0.02% | 0.00% | 0.00% | 0.01% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.01% | 0.01% | 0.01% | 0.00% | 0.00% | 0.01% | 0.01% | 0.00% |
| DdCBE_ | | | | | | | | | |
| replicate3 | | | | | | | | | |
| Batch | Nucleotide | A | C | T | A | C | C | A | C |
| ATP8- | A | 99.97% | 0.02% | 0.03% | 99.96% | 0.02% | 0.15% | 99.96% | 0.01% |
| DdCBE_ | | | | | | | | | |
| replicate 1 | | | | | | | | | |
| ATP8- | T | 0.01% | 0.04% | 99.95% | 0.01% | 0.03% | 0.04% | 0.01% | 0.02% |
| DdCBE_ | | | | | | | | | |
| replicate1 | | | | | | | | | |
| ATP8- | C | 0.01% | 99.94% | 0.02% | 0.02% | 99.95% | 99.80% | 0.00% | 99.97% |
| DdCBE_ | | | | | | | | | |
| replicate 1 | | | | | | | | | |
| ATP8- | G | 0.00% | 0.00% | 0.00% | 0.01% | 0.00% | 0.00% | 0.01% | 0.00% |
| DdCBE_ | | | | | | | | | |
| replicate 1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ATP8- | A | 99.97% | 0.02% | 0.01% | 99.96% | 0.03% | 0.13% | 99.96% | 0.02% |
| DdCBE_ | | | | | | | | | |
| replicate2 | | | | | | | | | |
| ATP8- | T | 0.01% | 0.04% | 99.96% | 0.01% | 0.01% | 0.06% | 0.02% | 0.02% |
| DdCBE_ | | | | | | | | | |
| replicate2 | | | | | | | | | |
| ATP8- | C | 0.01% | 99.93% | 0.02% | 0.01% | 99.96% | 99.81% | 0.01% | 99.96% |
| DdCBE_ | | | | | | | | | |
| replicate2 | | | | | | | | | |
| ATP8- | G | 0.01% | 0.00% | 0.00% | 0.01% | 0.00% | 0.00% | 0.00% | 0.00% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | A | 99.97% | 0.02% | 0.02% | 99.96% | 0.03% | 0.15% | 99.96% | 0.02% |
| DdCBE_ | | | | | | | | | |
| replicate3 | | | | | | | | | |
| ATP8- | T | 0.01% | 0.04% | 99.95% | 0.02% | 0.04% | 0.06% | 0.02% | 0.02% |
| DdCBE_ | | | | | | | | | |
| replicate3 | | | | | | | | | |
| ATP8- | C | 0.01% | 99.93% | 0.03% | 0.02% | 99.93% | 99.78% | 0.01% | 99.96% |
| DdCBE_ | | | | | | | | | |
| replicate3 | | | | | | | | | |
| ATP8- | G | 0.01% | 0.00% | 0.00% | 0.00% | 0.00% | 0.00% | 0.01% | 0.00% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.00% | 0.00% | 0.00% | 0.00% | 0.00% | 0.01% | 0.01% | 0.00% |
| DdCBE_ | | | | | | | | | |
| replicate3 | | | | | | | | | |
| Batch | Nucleotide | A | G | C | C | C | A | T | A |
| ATP8- | A | 99.97% | 0.06% | 0.02% | 0.02% | 0.06% | 99.95% | 0.02% | 99.98% |
| DdCBE_ | | | | | | | | | |
| replicate 1 | | | | | | | | | |
| ATP8- | T | 0.01% | 0.04% | 0.01% | 0.02% | 0.03% | 0.03% | 99.97% | 0.01% |
| DdCBE_ | | | | | | | | | |
| replicate 1 | | | | | | | | | |
| ATP8- | C | 0.01% | 0.06% | 99.97% | 99.95% | 99.91% | 0.01% | 0.01% | 0.00% |
| DdCBE_ | | | | | | | | | |
| replicate 1 | | | | | | | | | |
| ATP8- | G | 0.01% | 99.83% | 0.00% | 0.00% | 0.00% | 0.02% | 0.00% | 0.01% |
| DdCBE_ | | | | | | | | | |
| replicate1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.01% | 0.00% | 0.00% | 0.00% | 0.00% | 0.00% | 0.00% |
| DdCBE_ | | | | | | | | | |
| replicate1 | | | | | | | | | |
| ATP8- | A | 99.98% | 0.02% | 0.01% | 0.04% | 0.06% | 99.96% | 0.01% | 99.97% |
| DdCBE_ | | | | | | | | | |
| replicate2 | | | | | | | | | |
| ATP8- | T | 0.00% | 0.03% | 0.01% | 0.03% | 0.04% | 0.03% | 99.98% | 0.01% |
| DdCBE_ | | | | | | | | | |
| replicate2 | | | | | | | | | |
| ATP8- | C | 0.00% | 0.07% | 99.97% | 99.93% | 99.90% | 0.01% | 0.01% | 0.00% |
| DdCBE_ | | | | | | | | | |
| replicate2 | | | | | | | | | |
| ATP8- | G | 0.01% | 99.88% | 0.00% | 0.00% | 0.00% | 0.01% | 0.00% | 0.01% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.01% | 0.00% | 0.00% | 0.00% | 0.00% | 0.00% | 0.00% | 0.00% |
| DdCBE_ | | | | | | | | | |
| replicate2 | | | | | | | | | |
| ATP8- | A | 99.96% | 0.06% | 0.02% | 0.02% | 0.05% | 99.96% | 0.01% | 99.99% |
| DdCBE_ | | | | | | | | | |
| replicate3 | | | | | | | | | |
| ATP8- | T | 0.00% | 0.04% | 0.02% | 0.02% | 0.03% | 0.03% | 99.97% | 0.01% |
| DdCBE_ | | | | | | | | | |
| replicate3 | | | | | | | | | |
| ATP8- | C | 0.01% | 0.05% | 99.96% | 99.96% | 99.91% | 0.01% | 0.02% | 0.00% |
| DdCBE_ | | | | | | | | | |
| replicate3 | | | | | | | | | |
| ATP8- | G | 0.02% | 99.85% | 0.00% | 0.00% | 0.00% | 0.01% | 0.00% | 0.01% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.01% | 0.00% | 0.00% | 0.00% | 0.01% | 0.00% | 0.01% | 0.00% |
| DdCBE_ | | | | | | | | | |
| replicate3 | | | | | | | | | |
| Batch | Nucleotide | A | | C | | A | | A | |
| ATP8- | A | 99.97% | | 0.07% | | 99.98% | | 99.99% | |
| DdCBE_ | | | | | | | | | |
| replicate1 | | | | | | | | | |
| ATP8- | T | 0.01% | | 0.02% | | 0.00% | | 0.00% | |
| DdCBE_ | | | | | | | | | |
| replicate 1 | | | | | | | | | |
| ATP8- | C | 0.01% | | 99.91% | | 0.00% | | 0.00% | |
| DdCBE_ | | | | | | | | | |
| replicate 1 | | | | | | | | | |
| ATP8- | G | 0.01% | | 0.00% | | 0.01% | | 0.01% | |
| DdCBE_ | | | | | | | | | |
| replicate 1 | | | | | | | | | |
| ATP8- | N | 0.00% | | 0.00% | | 0.00% | | 0.00% | |
| DdCBE_ | | | | | | | | | |
| replicate 1 | | | | | | | | | |
| ATP8- | — | 0.00% | | 0.00% | | 0.00% | | 0.00% | |
| DdCBE_ | | | | | | | | | |
| replicate1 | | | | | | | | | |
| ATP8- | A | 99.94% | | 0.08% | | 99.99% | | 99.99% | |
| DdCBE_ | | | | | | | | | |
| replicate2 | | | | | | | | | |
| ATP8- | T | 0.02% | | 0.01% | | 0.00% | | 0.01% | |
| DdCBE_ | | | | | | | | | |
| replicate2 | | | | | | | | | |
| ATP8- | C | 0.03% | | 99.91% | | 0.00% | | 0.00% | |
| DdCBE_ | | | | | | | | | |
| replicate2 | | | | | | | | | |
| ATP8- | G | 0.01% | | 0.00% | | 0.00% | | 0.00% | |
| DdCBE_ | | | | | | | | | |
| replicate2 | | | | | | | | | |
| ATP8- | N | 0.00% | | 0.00% | | 0.00% | | 0.00% | |
| DdCBE_ | | | | | | | | | |
| replicate2 | | | | | | | | | |
| ATP8- | — | 0.01% | | 0.00% | | 0.00% | | 0.00% | |
| DdCBE_ | | | | | | | | | |
| replicate2 | | | | | | | | | |
| ATP8- | A | 99.94% | | 0.05% | | 99.98% | | 99.97% | |
| DdCBE_ | | | | | | | | | |
| replicate3 | | | | | | | | | |
| ATP8- | T | 0.02% | | 0.01% | | 0.00% | | 0.02% | |
| DdCBE_ | | | | | | | | | |
| replicate3 | | | | | | | | | |
| ATP8- | C | 0.02% | | 99.94% | | 0.00% | | 0.01% | |
| DdCBE_ | | | | | | | | | |
| replicate3 | | | | | | | | | |
| ATP8- | G | 0.01% | | 0.00% | | 0.01% | | 0.01% | |
| DdCBE_ | | | | | | | | | |
| replicate3 | | | | | | | | | |
| ATP8- | N | 0.00% | | 0.00% | | 0.00% | | 0.00% | |
| DdCBE_ | | | | | | | | | |
| replicate3 | | | | | | | | | |
| ATP8- | — | 0.01% | | 0.00% | | 0.00% | | 0.00% | |
| DdCBE_ | | | | | | | | | |
| replicate3 | | | | | | | | | |
| Batch | Nucleotide | A | A | A | A | T | A | T | T |
| ATP8- | A | 99.98% | 99.97% | 99.97% | 99.94% | 0.01% | 99.97% | 0.01% | 0.03% |
| DdCBE_ | | | | | | | | | |
| replicate 1 | | | | | | | | | |
| ATP8- | T | 0.01% | 0.02% | 0.01% | 0.01% | 99.99% | 0.01% | 99.98% | 99.95% |
| DdCBE_ | | | | | | | | | |
| replicate 1 | | | | | | | | | |
| ATP8- | C | 0.00% | 0.00% | 0.01% | 0.01% | 0.01% | 0.02% | 0.01% | 0.02% |
| DdCBE_ | | | | | | | | | |
| replicate1 | | | | | | | | | |
| ATP8 | G | 0.01% | 0.01% | 0.01% | 0.01% | 0.00% | 0.00% | 0.00% | 0.00% |
| DdCBE_ | | | | | | | | | |
| replicate 1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.00% | 0.00% | 0.03% | 0.00% | 0.00% | 0.00% | 0.00% |
| DdCBE_ | | | | | | | | | |
| replicate 1 | | | | | | | | | |
| ATP8- | A | 99.97% | 99.96% | 99.97% | 99.92% | 0.00% | 99.95% | 0.02% | 0.01% |
| DdCBE_ | | | | | | | | | |
| replicate2 | | | | | | | | | |
| ATP8- | T | 0.01% | 0.03% | 0.02% | 0.01% | 99.99% | 0.02% | 99.97% | 99.97% |
| DdCBE_ | | | | | | | | | |
| replicate2 | | | | | | | | | |
| ATP8- | C | 0.01% | 0.00% | 0.01% | 0.02% | 0.00% | 0.02% | 0.01% | 0.01% |
| DdCBE_ | | | | | | | | | |
| replicate2 | | | | | | | | | |
| ATP8- | G | 0.01% | 0.01% | 0.00% | 0.01% | 0.00% | 0.01% | 0.00% | 0.00% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.00% | 0.00% | 0.00% | 0.03% | 0.00% | 0.00% | 0.00% | 0.00% |
| DdCBE_ | | | | | | | | | |
| replicate2 | | | | | | | | | |
| ATP8- | A | 99.98% | 99.97% | 99.96% | 99.95% | 0.00% | 99.97% | 0.02% | 0.02% |
| DdCBE_ | | | | | | | | | |
| replicate3 | | | | | | | | | |
| ATP8- | T | 0.01% | 0.01% | 0.01% | 0.00% | 99.99% | 0.00% | 99.96% | 99.95% |
| DdCBE_ | | | | | | | | | |
| replicate3 | | | | | | | | | |
| ATP8- | C | 0.00% | 0.01% | 0.01% | 0.02% | 0.00% | 0.02% | 0.02% | 0.02% |
| DdCBE_ | | | | | | | | | |
| replicate3 | | | | | | | | | |
| ATP8- | G | 0.01% | 0.01% | 0.01% | 0.01% | 0.00% | 0.01% | 0.01% | 0.00% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.00% | 0.00% | 0.00% | 0.03% | 0.00% | 0.00% | 0.00% | 0.00% |
| DdCBE_ | | | | | | | | | |
| replicate3 | | | | | | | | | |
| Batch | Nucleotide | C | T | A | C | C | T | C | C |
| ATP8- | A | 0.01% | 0.03% | 99.95% | 0.02% | 0.02% | 0.01% | 0.01% | 0.01% |
| DdCBE_ | | | | | | | | | |
| replicate 1 | | | | | | | | | |
| ATP8- | T | 0.03% | 99.96% | 0.03% | 0.04% | 0.02% | 99.97% | 9.61% | 4.46% |
| DdCBE_ | | | | | | | | | |
| replicate1 | | | | | | | | | |
| ATP8- | C | 99.96% | 0.01% | 0.02% | 99.94% | 99.96% | 0.02% | 90.37% | 95.53% |
| DdCBE_ | | | | | | | | | |
| replicate 1 | | | | | | | | | |
| ATP8- | G | 0.00% | 0.00% | 0.00% | 0.00% | 0.00% | 0.00% | 0.00% | 0.00% |
| DdCBE_ | | | | | | | | | |
| replicate 1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.00% | 0.00% | 0.00% | 0.00% | 0.00% | 0.00% | 0.00% |
| DdCBE_ | | | | | | | | | |
| replicate 1 | | | | | | | | | |
| ATP8- | A | 0.03% | 0.02% | 99.93% | 0.01% | 0.03% | 0.02% | 0.03% | 0.01% |
| DdCBE_ | | | | | | | | | |
| replicate2 | | | | | | | | | |
| ATP8- | T | 0.02% | 99.95% | 0.03% | 0.02% | 0.02% | 99.97% | 12.09% | 5.71% |
| DdCBE_ | | | | | | | | | |
| replicate2 | | | | | | | | | |
| ATP8- | C | 99.95% | 0.03% | 0.02% | 99.97% | 99.94% | 0.01% | 87.88% | 94.27% |
| DdCBE_ | | | | | | | | | |
| replicate2 | | | | | | | | | |
| ATP8- | G | 0.00% | 0.00% | 0.02% | 0.00% | 0.00% | 0.00% | 0.00% | 0.00% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | A | 0.04% | 0.02% | 99.94% | 0.03% | 0.03% | 0.03% | 0.02% | 0.01% |
| DdCBE_ | | | | | | | | | |
| replicate3 | | | | | | | | | |
| ATP8- | T | 0.02% | 99.96% | 0.03% | 0.01% | 0.03% | 99.95% | 10.85% | 5.12% |
| DdCBE_ | | | | | | | | | |
| replicate3 | | | | | | | | | |
| ATP8- | C | 99.94% | 0.02% | 0.02% | 99.95% | 99.93% | 0.02% | 89.13% | 94.86% |
| DdCBE_ | | | | | | | | | |
| replicate3 | | | | | | | | | |
| ATP8- | G | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.00% | 0.00% | 0.00% | 0.00% | 0.00% | 0.00% | 0.00% | 0.00% |
| DdCBE_ | | | | | | | | | |
| replicate3 | | | | | | | | | |
| Batch | Nucleotide | A | A | A | A | T | A | A | A |
| ATP8- | A | 99.97% | 99.96% | 99.98% | 99.93% | 0.02% | 99.96% | 99.97% | 99.99% |
| DdCBE_ | | | | | | | | | |
| replicate 1 | | | | | | | | | |
| ATP8- | T | 0.01% | 0.01% | 0.00% | 0.03% | 99.97% | 0.01% | 0.02% | 0.00% |
| DdCBE_ | | | | | | | | | |
| replicate 1 | | | | | | | | | |
| ATP8- | C | 0.00% | 0.01% | 0.00% | 0.02% | 0.01% | 0.01% | 0.00% | 0.00% |
| DdCBE_ | | | | | | | | | |
| replicate 1 | | | | | | | | | |
| ATP8- | G | 0.01% | 0.02% | 0.01% | 0.00% | 0.00% | 0.01% | 0.01% | 0.01% |
| DdCBE_ | | | | | | | | | |
| replicate1 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate1 | | | | | | | | | |
| ATP8- | — | 0.00% | 0.00% | 0.00% | 0.01% | 0.00% | 0.00% | 0.00% | 0.00% |
| DdCBE_ | | | | | | | | | |
| replicate1 | | | | | | | | | |
| ATP8- | A | 99.97% | 99.97% | 99.98% | 99.90% | 0.02% | 99.97% | 99.97% | 99.99% |
| DdCBE_ | | | | | | | | | |
| replicate2 | | | | | | | | | |
| ATP8- | T | 0.02% | 0.01% | 0.01% | 0.04% | 99.96% | 0.01% | 0.02% | 0.01% |
| DdCBE_ | | | | | | | | | |
| replicate2 | | | | | | | | | |
| ATP8- | C | 0.00% | 0.00% | 0.00% | 0.01% | 0.01% | 0.01% | 0.00% | 0.00% |
| DdCBE_ | | | | | | | | | |
| replicate2 | | | | | | | | | |
| ATP8- | G | 0.00% | 0.01% | 0.00% | 0.02% | 0.00% | 0.01% | 0.01% | 0.01% |
| DdCBE_ | | | | | | | | | |
| replicate2 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate2 | | | | | | | | | |
| ATP8- | — | 0.00% | 0.00% | 0.00% | 0.03% | 0.00% | 0.00% | 0.00% | 0.00% |
| DdCBE_ | | | | | | | | | |
| replicate2 | | | | | | | | | |
| ATP8- | A | 99.97% | 99.97% | 99.97% | 99.91% | 0.01% | 99.97% | 99.98% | 99.99% |
| DdCBE_ | | | | | | | | | |
| replicate3 | | | | | | | | | |
| ATP8- | T | 0.01% | 0.02% | 0.01% | 0.04% | 99.96% | 0.01% | 0.01% | 0.01% |
| DdCBE_ | | | | | | | | | |
| replicate3 | | | | | | | | | |
| ATP8- | C | 0.01% | 0.00% | 0.01% | 0.02% | 0.02% | 0.01% | 0.00% | 0.00% |
| DdCBE_ | | | | | | | | | |
| replicate3 | | | | | | | | | |
| ATP8- | G | 0.01% | 0.01% | 0.01% | 0.00% | 0.00% | 0.01% | 0.00% | 0.00% |
| DdCBE_ | | | | | | | | | |
| replicate3 | | | | | | | | | |
| ATP8- | N | 0.00% | 0.00% | 0.00% | 0.00% | 0.00% | 0.00% | 0.00% | 0.00% |
| DdCBE_ | | | | | | | | | |
| replicate3 | | | | | | | | | |
| ATP8- | — | 0.00% | 0.00% | 0.00% | 0.03% | 0.01% | 0.00% | 0.00% | 0.00% |
| DdCBE_ | | | | | | | | | |
| replicate3 | | | | | | | | | |

### Supplementary Sequences

Sequences used for DddAₜₒₓ characterization in bacteria.

| | | SEQ ID NOS |
|---|---|---|
| Primers for cloning dddA deaminase domain into pScrhaB2-V | | |
| NdeI-DddA | TCAAGTACTACATATGATAGGACTCAACGGTGGGGC | 160 |
| DddA-NS- | TACTGATTGATCTAGAACAACCTCCTTTCGTGGGGGA | 161 |
| XbaI | | |
| Primers for cloning dddA deaminase domain into pPSV39-CV | | |
| DddAI- | TACTGATTGATCTAGATTACAACTCGCTCCATGTCAGTTG | 162 |
| XbaI | | |
| SacI-RBS- | TCAAGTACTAGAGCTCACGGGAGGAAAGATGTACGCAGACG | 163 |
| DddAI | ATTTCGACG | |
| Primers for cloning dddA deaminase domain into pETDuet-1 mcs1 | | |
| BamHI- | TATCAGAAACGGATCCATAGGACTCAACGGTGGGGC | 164 |
| DddA_duet | | |
| DddA_duet- | TATGTTACTAGCGGCCGCTCAACAACCTCCTTTCGTGGG | 165 |
| NotI | | |
| Primers for cloning dddA deaminase domain into pETDuet-1 mcs1 | | |
| NdeI_DddAI | TCAAGTACTACATATGTACGCAGACGATTTCGACG | 166 |
| DddAI-BglII | TATGTTACTAAGATCTTTACAACTCGCTCCATGTCAGTTG | 167 |
| Primers for cloning dddA(E1347A) deaminase domain into pETDuet-1 mcs1 | | |
| DddA_E1347A- | CGGACTGACCGGCAACGTGCCCGGCGTTTGC | 168 |
| 3 | | |
| DddA_E1347A- | GGCACGTTGCCGGTCAGTCCGCCTTATTTATGC | 169 |
| 4 | | |
| Primers for construction of deletion cassette for icmF1 using | | |
| pDONRPEX18Gm-SceI-pheS | | |
| HindIII- | TGTTAAGCTAAAGCTTTAGGGATAACAGGGTAATCTGCTGG | 170 |
| ISecI-IcmF1 | ATCCGGATTTCCG | |
| IcmF1-2 | TTCAGCATGCTTGCGGCTCGAGTTGATGCGTTGCATAGGAC | 171 |
| | GTTCA | |
| Icmf1-3 | AACTCGAGCCGCAAGCATGCTGAAAGGGCGCAATGACGCAA | 172 |
| | ACC | |
| Bcen_IcmF1- | TCAATCAGTATCTAGAGTAGAACGGATCGACCGGCA | 173 |
| XbaI | | |
| HindIII- | TGTTAAGCTAAAGCTTTAGGGATAACAGGGTAATCGCTCAT | 174 |
| ISecI-IcmF2 | TGTCCGTTTGCAGC | |
| IcmF2-2 | TTCAGCATGCTTGCGGCTCGAGTTGCGAACGATCATGTGTG | 175 |
| | ATACAC | |
| IcmF2-3 | AACTCGAGCCGCAAGCATGCTGAATTTCGAGACCCGCGATG | 176 |
| | ACG | |
| IcmF2-XbaI | TCAATCAGTATCTAGACGAGCCGCTCGATACGATTG | 177 |
| Primers for construction of deletion cassette for dddA-dddAr using | | |
| pDONRPEX18Gm-Scel-pheS | | |
| HindIII- | TGTTAAGCTAAAGCTTTAGGGATAACAGGGTAATGTGGTAC | 178 |
| ISecI- | TTCAACGAAGCAGATG | |
| DddADddAI | | |
| DddADddAI- | TTCAGCATGCTTGCGGCTCGAGTTATCGGATCAGTGACTCG | 179 |
| 2 | TGC | |
| DddADddA | AACTCGAGCCGCAAGCATGCTGAAAGCGAGTTGTAAGAAAC | 180 |
| I-3 | GGAGC | |
| Bcen_E1- | TCAATCAGTATCTAGAAGTGAGCTCTCCGAAATCGAAC | 181 |
| I1-XbaI | | |
| Primers for construction of dddA(E147A) using pDONRPEX18Gm-SceI-pheS | | |
| Bcen_E1347A- | TAAAACGACGGCCAGTGCCAAGCTTAGGGATAACAGGGTAA | 182 |
| 1 | TAGCAGCTACGTGTACAGTCCGGACGCACCGTATTCGC | |
| Bcen_E1347A- | AATAAGGCGGACTGACCGGCAACGTGCCCGGCGTTTGCGTA | 183 |
| 2 | GTTGG | |
| Bcen_E1347A- | CCGGGCACGTTGCCGGTCAGTCCGCCTTATTTATGCGC | 184 |
| 3 | | |
| Bcen_E1347A- | GCTCGGTACCCGGGGATCCTCTAGACTCGCTCCATGTCAGT | 185 |
| 4 | TGCTCGGGCCG | |
| Primers for cloning of cdd into pScrhaB2-V | | |
| CDD_fwd | TGAAATTCAGCAGGATCACATATGCATCCACGTTTTCAAAC | 186 |
| | CGC | |
| CDD_rev | TGCATGCCTGCAGGTCGACTCTAGAAGCGAGAAGCACTCGG | 187 |
| | TC | |
| Primers for cloning of tadA into pScrhaB2-V | | |
| tadA fwd | AATGATACGGCGACCACCGAGATCTACACTCTTTCCCTACA | 188 |
| | CGACGCTCTTCCGATCT | |
| tadA_rev | TGCATGCCTGCAGGTCGACTCTAGACTTAAAAATACGTATC | 189 |
| | GCTTTAG | |
| Primers for cloning of A3G into pScrhaB2-V | | |
| A3G_fwd | TGAAATTCAGCAGGATCACATATGGACCCACCAACTTTTAC | 190 |
| | TTTTAATTTTAAC | |
| A3G_rev | TGCCTGCAGGTCGACTCTAGAGTTTTCCTGGTTTTGTAAGA | 191 |
| | TTG | |
| Primers for ung deletion in E.coli | | |
| ung_del-F | TAGAAAGAAGCAGTTAAGCTAGGCGGATTGAAGATTCGCAG | 192 |
| | GAGAGCGAGATGGTGTAGGCTGGAGCTGCTTC | |
| ung_del-R | TGATAAATCAGCCGGGGGCAACTCTGCCATCCGGCATTTC | 193 |
| | CCCGCAAATTTACATATGAATATCCTCCTTAG | |
| Primers for cloning ung into pBAD24 | | |
| EcoRI_ung | TAGTACAGAGAATTCATGGCTAACGAATTAACCTGGCATGA | 194 |
| | C | |
| ung_XbaI | TCAATCAGTATCTAGATTACTCACTCTCTGCCGGTAATACT | 195 |
| | G | |
| Sequences for in vitro deamination assays | | |
| S36- | AAAAAAAAAAAAAAACTCGCCAAAAAAAAAAAAAAA | 196 |
| CTCGCC | | |
| S35- | AAAAAAAAAAAAAAAGACGGAAAAAAAAAAAAAAA | 197 |
| GACGG | | |
| S35- | AAAAAAAAAAAAAAAGTCGGAAAAAAAAAAAAAAA | 198 |
| GTCGG | | |
| S35- | AAAAAAAAAAAAAAAGGCGGAAAAAAAAAAAAAAA | 199 |
| GGCGG | | |
| S35- | AAAAAAAAAAAAAAAGCCGGAAAAAAAAAAAAAAA | 200 |
| GCCGG | | |

Sequences used for characterization of DddAₜₒₓ and its fusions in mammalian cells.

| | | SEQ ID NOS |
|---|---|---|
| Primers used for generating sgRNA transfection plasmids. | | |
| Rev_sgRNA_plasmid was used in all cases | | |
| rev_sgRNA_ | GGTGTTTCGTCCTTTCCACAAG | 201 |
| plasmid | | |
| fwd_saG1 | GTCTGTGCCCCTCCCTCCCTGGCGTTTTAGTACTCTGT | 202 |
| | AATGAAAATTACAGAATCTAC | |
| fwd_saG2 | GCCCCTCCCTCCCTGGCCCAGGTGTTTTAGTACTCTGT | 203 |
| | AATGAAAATTACAGAATCTAC | |
| fwd_saG3 | GCCCTCCCTGGCCCAGGTGAAGGGTTTTAGTACTCTGT | 204 |
| | AATGAAAATTACAGAATCTAC | |
| fwd_saG4 | GTGTGGTTCCAGAACCGGAGGAGTTTTAGTACTCTGTA | 205 |
| | ATGAAAATTACAGAATCTAC | |
| fwd_spG6 | GAGGCCCCCAGAGCAGCCACGTTTTAGAGCTAGAAATA | 206 |
| | GCAAGTTAAAATAAGGC | |
| fwd_spG7 | GCCACTGGGGCCTCAACACTCGTTTTAGAGCTAGAAAT | 207 |
| | AGCAAGTTAAAATAAGGC | |
| fwd_EMX1 | GAGTCCGAGCAGAAGAAGAAGTTTTAGAGCTAGAAATA | 208 |
| | GCAAGTTAAAATAAGGC | |
| Primers for HTS of on-target sites from all mammalian cell culture | | |
| experiments | | |
| fwd_CCR5_HTS | ACACTCTTTCCCTACACGACGCTCTTCCGATCTNNNNC | 209 |
| | AAGTGTGATCACTTGGGTGG | |
| rev_CCR5_HTS | TGGAGTTCAGACGTGTGCTCTTCCGATCTGGATTCCCG | 210 |
| | AGTAGCAGATG | |
| fwd_EMX1_HTS | ACACTCTTTCCCTACACGACGCTCTTCCGATCTNNNNG | 211 |
| | GCCCCTAACCCTATGTAGC | |
| rev_EMX1_HTS | TGGAGTTCAGACGTGTGCTCTTCCGATCTCTTCTGCTC | 212 |
| | GGACTCAGGC | |
| fwd_ND1_HTS | ACACTCTTTCCCTACACGACGCTCTTCCGATCTNNNNC | 213 |
| | TCACCATCGCTCTTCTACTATG | |
| rev_ND1_HTS | TGGAGTTCAGACGTGTGCTCTTCCGATCTGGCTAGGGT | 214 |
| | GACTTCATATGAG | |
| fwd_ND2_HTS | ACACTCTTTCCCTACACGACGCTCTTCCGATCTNNNNC | 215 |
| | GTAAGCCTTCTCCTCACT | |
| rev_ND2_HTS | TGGAGTTCAGACGTGTGCTCTTCCGATCTGTTGAGTAG | 216 |
| | TAGGAATGCGGTAG | |
| fwd_ND4_HTS | ACACTCTTTCCCTACACGACGCTCTTCCGATCTNNNNG | 217 |
| | ACTTCAAACTCTACTCCCACTAATAG | |
| rev_ND4_HTS | TGGAGTTCAGACGTGTGCTCTTCCGATCTGTTGTGGTA | 218 |
| | AATATGTAGAGGGAG | |
| fwd_ND5.1/5.2 | ACACTCTTTCCCTACACGACGCTCTTCCGATCTNNNNC | 219 |
| HTS | GGGTCCATCATCCACAAC | |
| rev_ND5.1/5.2 | TGGAGTTCAGACGTGTGCTCTTCCGATCTAGAGTAATA | 220 |
| HTS | GATAGGGCTCAGGC | |
| fwd_ND5.3_HTS | ACACTCTTTCCCTACACGACGCTCTTCCGATCTNNNNC | 221 |
| | TGTAGCATTGTTCGTTACATGG | |
| rev_ND5.3_HTS | TGGAGTTCAGACGTGTGCTCTTCCGATCTCTGATGAGC | 222 |
| | AAGAAGGATATAATTCC | |
| fwd_ND6_HTS | ACACTCTTTCCCTACACGACGCTCTTCCGATCTNNNNC | 223 |
| | TCTTTCACCCACAGCACC | |
| rev_ND6_HTS | TGGAGTTCAGACGTGTGCTCTTCCGATCTGATTGTTAG | 224 |
| | CGGTGTGGTCG | |
| fwd_ATP8_HTS | ACACTCTTTCCCTACACGACGCTCTTCCGATCTNNNNC | 225 |
| | TTTACAGTGAAATGCCCCAAC | |
| rev_ATP8_HTS | TGGAGTTCAGACGTGTGCTCTTCCGATCTGGGGGCAAT | 226 |
| | GAATGAAGCG | |
| Primers for HTS of off-target sites from all mammalian cell culture | | |
| experiments | | |
| fwd_5303_HTS | ACACTCTTTCCCTACACGACGCTCTTCCGATCTNNNNG | 227 |
| | CTAACATGACTAACACCCTTAATTC | |
| rev_5303_HTS | TGGAGTTCAGACGTGTGCTCTTCCGATCTGTAGGAGTA | 228 |
| | GCGTGGTAAGG | |
| fwd_7994/8115_ | ACACTCTTTCCCTACACGACGCTCTTCCGATCTNNNNC | 229 |
| HTS | CCCCATTATTCCTAGAACCAG | |
| rev_7994/8115_ | TGGAGTTCAGACGTGTGCTCTTCCGATCTGCTATAGGG | 230 |
| HTS | TAAATACGGGCC | |
| fwd_8619/8648/ | ACACTCTTTCCCTACACGACGCTCTTCCGATCTNNNNG | 231 |
| 8720_HTS | ATCATTCTATTTCCCCCTCTATTG | |
| rev_8619/8648/ | TGGAGTTCAGACGTGTGCTCTTCCGATCTCACTGTGCC | 232 |
| 8720_HTS | CGCTCATAAG | |
| fwd_10192/10205/ | ACACTCTTTCCCTACACGACGCTCTTCCGATCTNNNNG | 233 |
| 10349_HTS | AAAAATCCACCCCTTACGAG | |
| rev_10192/10205/ | TGGAGTTCAGACGTGTGCTCTTCCGATCTCGTTTTGTT | 234 |
| 10349_HTS | TAAACTATATACCAATTCGG | |
| fwd_13763_HTS | ACACTCTTTCCCTACACGACGCTCTTCCGATCTNNNNC | 235 |
| | CCCACCCTTACTAACATTAACG | |
| rev_13763_HTS | TGGAGTTCAGACGTGTGCTCTTCCGATCTGTTTGTTGG | 236 |
| | TTAGGTAGTTGAGG | |
| fwd_15598/15619/ | ACACTCTTTCCCTACACGACGCTCTTCCGATCTNNNNC | 237 |
| 15646/15675 | CTATTCGCCTACACAATTCTC | |
| HTS | | |
| rev_15598/15619/ | TGGAGTTCAGACGTGTGCTCTTCCGATCTGTTGGTATT | 238 |
| 15646/15675 | AGGATTAGGATTGTTGTG | |
| HTS | | |
| fwd_15950_HTS | ACACTCTTTCCCTACACGACGCTCTTCCGATCTNNNNC | 239 |
| | TCAAATGGGCCTGTCCTTG | |
| rev_15950_HTS | TGGAGTTCAGACGTGTGCTCTTCCGATCTGTACTACAG | 240 |
| | GTGGTCAAGTATTTATG | |
| fwd_16363/16393/ | ACACTCTTTCCCTACACGACGCTCTTCCGATCTNNNNC | 241 |
| 16394_HTS | CATTTACCGTACATAGCACATTAC | |
| rev_16363/16393/ | TGGAGTTCAGACGTGTGCTCTTCCGATCTCGTGAGTGG | 242 |
| 16394_HTS | TTAATAGGGTGATAG | |
| Primers for mtDNA copy number anaylsis by quantitative PCR | | |
| fwd_ND5.1/5.2_ | GAACAAGATATTCGAAAAATAGGAGGAC | 243 |
| qPCR | | |
| rev_ND5.1/5.2_ | GCGGTTTCGATGATGTGG | 244 |
| qPCR | | |
| fwd_ND6_qPCR | CACTCACCAAGACCTCAACC | 245 |
| rev_ND6_qPCR | GAATGATGGTTGTCTTTGGATATACTAC | 246 |
| fwd_ATP8_qPCR | CTTACACTATTCCTCATCACCCAAC | 247 |
| rev_ATP8_qPCR | GTTCATTTTGGTTCTCAGGGTTTG | 248 |
| fwd_ß- | AGGCACCAGGGCGTGAT | 249 |
| actin_qPCR | | |
| rev_ß- | CAGGGTGAGGATGCCTC | 250 |
| actin_qPCR | | |
| Primers for long-range PCR of whole mitochondrial genome as two | | |
| amplicons | | |
| fwd_2478-10858 | GCAAATCTTACCCCGCCTG | 251 |
| rev_2478-10858 | AATTAGGCTGTGGGTGGTTG | 252 |
| fwd_2688-10653 | GCCATACTAGTCTTTGCCGC | 253 |
| rev_2688-10653 | GGCAGGTCAATTTCACTGG | 254 |
| Primer for amplification of ND4 gene fragement from ND4-edited cells | | |
| fwd_ND4_PCR | GCCATTCTCATCCAAACC | 255 |
| Rev_ND4_PCR | GGTTGAGGGATAGGAGGAG | 256 |
| Primers for qPCR and RT-qPCR of ND4-edited cells | | |
| fwd_ND4_RT- | CAAGCTCCATCTGCCTACGA | 257 |
| qPCR | | |
| rev_ND4_RT- | GCGATTATGAGAATGACTGC | 258 |
| qPCR | | |
| fwd_RNR1_RT- | ATTACACATGCAAGCATCCC | 259 |
| qPCR | | |
| rev_RNR1_RT- | CACGAAATTGACCAACCCTG | 260 |
| qPCR | | |
| fwd_ND1_RT- | TAGCAGAGACCAACCGAACC | 261 |
| qPCR | | |
| rev_ND1_RT- | ATGAAGAATAGGGCGAAGGG | 262 |
| qPCR | | |
| fwd_ND2_RT- | CTATCTCGCACCTGAAACAAGC | 263 |
| qPCR | | |
| rev_ND2_RT- | GGTGGAGTAGATTAGGCGTAGG | 264 |
| qPCR | | |
| fwd_ATP6/8_ | TGTTAGCGGTTAGGCGTA | 265 |
| RT-qPCR | | |
| rev_ATP6/8_ | TTACACCAACCACCCAAC | 266 |
| RT-qPCR | | |
| fwd_CO3_RT- | TTTACCCTCCTACAAGCC | 267 |
| qPCR | | |
| rev_CO3_RT- | GCGGATGAAGCAGATAGT | 268 |
| qPCR | | |
| fwd_CYB_RT- | GCCTGCCTGATCCTCCAAAT | 269 |
| qPCR | | |
| rev_CYB_RT- | AAGGTAGCGGATGATTCAGCC | 270 |
| qPCR | | |
| fwd_B2M_RT- | CAGGTACTCCAAAGATTCAGG | 271 |
| qPCR_qPCR | | |
| rev_B2M_RT- | GTCAACTTCAATGTCGGATGG | 272 |
| qPCR_qPCR | | |
| Nextera primers for ATAC-seq | | |
| i5_common | AATGATACGGCGACCACCGAGATCTACACTCGTCGGCA | 273 |
| | GCGTCAGATGTG | |
| i7_1 | CAAGCAGAAGACGGCATACGAGATTCGCCTTAGTCTCG | 274 |
| | TGGGCTCGGAGATGT | |
| i7_2 | CAAGCAGAAGACGGCATACGAGATCTAGTACGGTCTCG | 275 |
| | TGGGCTCGGAGATGT | |
| i7_3 | CAAGCAGAAGACGGCATACGAGATTTCTGCCTGTCTCG | 276 |
| | TGGGCTCGGAGATGT | |
| i7_4 | CAAGCAGAAGACGGCATACGAGATGCTCAGGAGTCTCG | 277 |
| | TGGGCTCGGAGATGT | |
| i7_5 | CAAGCAGAAGACGGCATACGAGATAGGAGTCCGTCTCG | 278 |
| | TGGGCTCGGAGATGT | |
| i7_6 | CAAGCAGAAGACGGCATACGAGATCATGCCTAGTCTCG | 279 |
| | TGGGCTCGGAGATGT | |
| i7_7 | CAAGCAGAAGACGGCATACGAGATGTAGAGAGGTCTCG | 280 |
| | TGGGCTCGGAGATGT | |
| i7_8 | CAAGCAGAAGACGGCATACGAGATCCTCTCTGGTCTCG | 281 |
| | TGGGCTCGGAGATGT | |
| i7_9 | CAAGCAGAAGACGGCATACGAGATAGCGTAGCGTCTCG | 282 |
| | TGGGCTCGGAGATGT | |

### Split-DDAₜₒₓ-Cas9 Fusions Sequences

G1397 DddAₜₒₓ-N
(SEQ ID NO: 283)
GGCAGCTACGCCCTGGGTCCGTATCAGATTAGCGCCCCGCAGCTGCCAGCTTACAATGGTCAGA
CCGTGGGTACCTTCTACTATGTGAACGACGCGGGCGGTCTGGAGAGCAAGGTGTTTAGCAGCGG
CGGTCCAACCCCGTACCCAAACTATGCCAATGCCGGTCATGTGGAGGGTCAGAGCGCCCTGTTC
ATGCGTGATAACGGCATCAGCGAGGGTCTGGTGTTCCACAACAACCCGGAAGGCACCTGCGGTT
TTTGCGTGAACATGACCGAGACCCTGCTGCCGGAAAACGCGAAAATGACCGTGGTGCCGCCGGA
AGGT
Translated amino acid sequence:
(SEQ ID NO: 284)
GSYALGPYQISAPOLPAYNGQTVGTFYYVNDAGGLESKVFSSGGPTPYPNYANAGHVEGQSALF
MRDNGISEGLVFHNNPEGTCGFCVNMTETLLPENAKMTVVPPEG
G1397 DddAₜₒₓ-C
(SEQ ID NO: 285)
GCCATTCCAGTGAAGCGCGGCGCTACCGGTGAAACCAAAGTGTTTACCGGTAACAGCAACAGCC
CGAAGAGCCCGACCAAAGGCGGTTGC
(SEQ ID NO: 286)
Translated amino acid sequence:
AIPVKRGATGETKVFTGNSNSPKSPTKGGC
G1333 DddAₜₒₓ-N-SaKKH-Cas9(D10A)-UGI
(SEQ ID NO: 287)
GGCAGCTACGCCCTGGGTCCGTATCAGATTAGCGCCCCGCAGCTGCCAGCTTACAATGGTCAGA
CCGTGGGTACCTTCTACTATGTGAACGACGCGGGCGGTCTGGAGAGCAAGGTGTTTAGCAGCGG
CGGTTCTGGTGGTTCTTCTGGTGGTTCTAGCGGCAGCGAGACTCCCGGGACCTCAGAGTCCGCC
ACACCCGAAAGTAGTGGCGGCAGCAGCGGCGGCAGCGGGAAACGGAACTACATCCTGGGGCTTG
CCATTGGGATAACCAGCGTTGGCTACGGAATTATTGATTATGAGACACGCGATGTGATTGACGC
CGGGGTTAGGCTGTTCAAAGAGGCCAACGTTGAAAACAACGAGGGAAGACGGAGTAAGCGCGGA
GCAAGAAGACTCAAGCGCAGACGGAGACATCGGATTCAGAGGGTGAAAAAGCTGCTCTTCGATT
ACAATCTCCTGACCGATCATAGTGAGCTGAGCGGAATCAACCCCTACGAGGCGCGAGTGAAAGG
GCTTTCCCAGAAGCTGTCCGAAGAGGAGTTCTCCGCCGCGTTGCTGCACCTGGCCAAACGGAGG
GGGGTTCACAATGTAAACGAAGTGGAGGAGGACACGGGCAATGAACTTAGTACGAAAGAACAGA
TCAGTAGGAACTCTAAGGCTCTCGAAGAGAAATACGTCGCTGAGTTGCAGCTTGAGAGACTGAA
AAAAGACGGCGAAGTACGCGGATCTATTAATAGGTTCAAGACTTCAGATTACGTAAAGGAAGCC
AAGCAGCTCCTGAAAGTACAGAAAGCGTACCATCAGCTCGATCAGAGCTTCATCGATACCTACA
TAGATTTGCTGGAGACACGGAGGACATACTACGAGGGCCCAGGGGAAGGATCTCCTTTTGGGTG
GAAGGACATCAAGGAATGGTACGAGATGCTTATGGGACATTGTACATATTTTCCGGAGGAGCTC
AGGAGCGTCAAGTACGCCTACAATGCCGACCTGTACAATGCCCTCAATGACCTCAATAACCTCG
TGATTACCAGGGACGAGAACGAGAAGCTGGAGTACTATGAAAAGTTCCAGATTATCGAGAATGT
GTTTAAGCAGAAGAAGAAGCCGACACTTAAGCAGATTGCAAAGGAAATCCTCGTGAATGAGGAA
GATATCAAGGGATACAGAGTGACAAGTACAGGCAAGCCCGAGTTCACAAATCTGAAGGTGTACC
ACGATATTAAGGACATAACCGCACGAAAGGAGATAATCGAAAACGCTGAGCTCCTCGATCAGAT
CGCAAAAATTCTTACCATCTACCAGTCTAGTGAGGACATTCAGGAGGAACTGACTAATCTGAAC
AGTGAGCTCACCCAAGAGGAAATTGAGCAGATTTCAAACCTGAAAGGCTACACCGGGACGCACA
ATCTGAGCCTCAAAGCAATCAACCTCATTCTGGATGAACTTTGGCACACAAATGACAACCAAAT
TGCCATATTCAACCGCCTGAAACTGGTGCCAAAAAAAGTGGATCTGTCACAGCAAAAGGAAATC
CCTACAACCTTGGTTGACGATTTTATTCTGTCCCCCGTTGTCAAGCGGAGCTTCATCCAGTCAA
TCAAGGTGATCAATGCCATCATTAAAAAATACGGATTGCCAAACGATATAATTATCGAGCTTGC
ACGAGAGAAGAACTCAAAGGACGCCCAGAAGATGATTAACGAAATGCAGAAGCGCAACCGCCAG
ACAAACGAACGCATAGAGGAAATTATAAGAACAACCGGCAAAGAGAATGCCAAGTATCTGATCG
AGAAAATCAAGCTGCACGACATGCAAGAAGGCAAGTGCCTGTACTCTCTGGAAGCTATCCCACT
CGAAGATCTGCTGAATAATCCATTCAATTACGAGGTGGACCACATCATCCCTAGATCCGTAAGC
TTTGACAATTCCTTCAATAACAAAGTTCTGGTTAAACAGGAGGAAAATTCTAAAAAAGGGAACC
GGACCCCGTTCCAGTACCTGAGCTCCAGTGACAGCAAGATTAGCTACGAGACTTTTAAGAAACA
TATTCTGAATCTGGCCAAAGGCAAAGGCAGGATCAGCAAGACCAAGAAGGAGTACCTCCTCGAA
GAACGCGACATTAACAGATTTAGTGTGCAGAAAGATTTCATCAACCGAAACCTTGTCGATACTC
GGTACGCCACGAGAGGCCTGATGAATCTCCTCAGGAGCTACTTCCGCGTCAATAATCTGGACGT
TAAAGTCAAGAGCATAAATGGGGGATTCACCAGCTTTCTGAGGAGAAAGTGGAAGTTTAAGAAG
GAACGAAACAAAGGATACAAGCACCATGCTGAGGATGCTTTGATCATCGCTAACGCGGACTTTA
TCTTTAAGGAATGGAAAAAGCTGGATAAGGCAAAGAAAGTGATGGAAAACCAGATGTTCGAGGA
GAAGCAGGCAGAGTCAATGCCTGAGATCGAGACAGAGCAGGAATACAAGGAAATTTTCATCACC
CCTCATCAGATTAAACACATAAAGGACTTCAAAGACTATAAATACTCTCATAGGGTGGACAAAA
AACCCAATCGCAAGCTCATTAATGACACCCTGTACTCAACACGGAAGGATGATAAAGGTAATAC
CTTGATTGTGAATAATCTTAATGGATTGTATGACAAAGATAACGACAAGCTCAAGAAGCTGATC
AACAAGTCTCCAGAGAAGCTCCTTATGTATCACCACGACCCACAGACTTATCAGAAATTGAAAC
TGATCATGGAGCAATACGGGGATGAGAAGAACCCACTCTACAAATATTATGAGGAAACAGGTAA
TTACCTGACCAAGTACTCCAAGAAGGATAACGGACCAGTGATCAAAAAGATAAAGTACTATGGC
AACAAACTTAATGCGCATTTGGACATAACTGACGATTACCCCAATTCTCGAAACAAGGTTGTGA
AGCTCTCCCTGAAGCCTTATAGATTTGACGTGTACCTGGATAATGGGGTTTATAAATTCGTCAC
CGTGAAAAATCTGGACGTGATCAAAAAGGAGAACTATTATGAAGTAAACTCAAAGTGCTATGAG
GAGGCGAAGAAGCTGAAGAAGATCTCCAATCAGGCCGAGTTCATCGCTTCCTTCTATAAGAACG
ATCTCATCAAGATCAATGGAGAGCTTTATCGCGTCATTGGTGTGAACAATGACTTGCTGAACAG
GATCGAAGTCAATATGATAGACATTACCTACCGGGAGTATCTCGAAAACATGAATGATAAACGG
CCGCCTCACATCATCAAGACAATCGCATCTAAAACTCAGTCAATAAAAAAGTACTCTACCGATA
TCCTGGGGAATCTCTATGAAGTGAAGTCAAAGAAGCACCCACAAATCATTAAAAAAGGTGGATC
CCCCAAGAAGAAGAGGAAAGTCTCGAGCGACTACAAAGACCATGACGGTGATTATAAAGATCAT

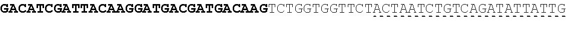

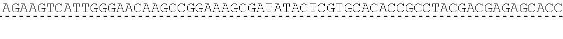

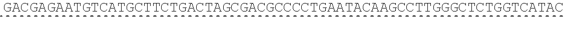

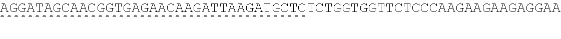

AGTC
Translated amino acid sequence:
(SEQ ID NO: 288)
GSYALGPYQISAPQLPAYNGOTVGTFYYVNDAGGLESKVFSSGGSGGSSGGSSGSETPGTSESA
TPESSGGSSGGSGKRNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSEEEFSAALLHLAKRR
GVHNVNEVEEDTGNELSTKEQISRNSKALEEKYVAELQLERLKKDGEVRGSINRFKTSDYVKEA
KQLLKVOKAYHOLDQSFIDTYIDLLETRRTYYEGPGEGSPFGWKDIKEWYEMLMGHCTYFPEEL
RSVKYAYNADLYNALNDLNNLVITRDENEKLEYYEKFQIIENVFKQKKKPTLKQIAKEILVNEE
DIKGYRVTSTGKPEFTNLKVYHDIKDITARKEIIENAELLDQIAKILTIYQSSEDIQEELTNLN
SELTQEEIEQISNLKGYTGTHNLSLKAINLILDELWHINDNQIAIFNRLKLVPKKVDLSQQKEI
PTTLVDDFILSPVVKRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNPFNYEVDHIIPRSVS
FDNSFNNKVLVKQEENSKKGNRTPFQYLSSSDSKISYETFKKHILNLAKGKGRISKTKKEYLLE
ERDINRFSVQKDFINRNLVDTRYATRGLMNLLRSYFRVNNLDVKVKSINGGFTSFLRRKWKFKK
ERNKGYKHHAEDALIIANADFIFKEWKKLDKAKKVMENOMFEEKQAESMPEIETEQEYKEIFIT
PHQIKHIKDFKDYKYSHRVDKKPNRKLINDTLYSTRKDDKGNTLIVNNLNGLYDKDNDKLKKLI
NKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYE
EAKKLKKISNQAEFIASFYKNDLIKINGELYRVIGVNNDLLNRIEVNMIDITYREYLENMNDKR
PPHIIKTIASKTQSIKKYSTDILGNLYEVKSKKHPQIIKKGGSPKKKRKVSSDYKDHDGDYKDH
DIDYKDDDDKSGGSTNLSDIIEKETGKQLVIQESILMLPEEVEEVIGNKPESDILVHTAYDEST
DENVMLLTSDAPEYKPWALVIQDSNGENKIKMLSGGSPKKKRKV
G1333 DddAₜₒₓ-C-SaKKH-Cas9(D10A)-UGI
(SEQ ID NO: 289)
CCAACCCCGTACCCAAACTATGCCAATGCCGGTCATGTGGAGGGTCAGAGCGCCCTGTTCATGC
GTGATAACGGCATCAGCGAGGGTCTGGTGTTCCACAACAACCCGGAAGGCACCTGCGGTTTTTG
CGTGAACATGACCGAGACCCTGCTGCCGGAAAACGCGAAAATGACCGTGGTGCCGCCGGAAGGT
GCCATTCCAGTGAAGCGCGGCGCTACCGGTGAAACCAAAGTGTTTACCGGTAACAGCAACAGCC
CGAAGAGCCCGACCAAAGGCGGTTGCTCTGGTGGTTCTTCTGGTGGTTCTAGCGGCAGCGAGAC
TCCCGGGACCTCAGAGTCCGCCACACCCGAAAGTAGTGGCGGCAGCAGCGGCGGCAGCGGGAAA
CGGAACTACATCCTGGGGCTTGCCATTGGGATAACCAGCGTTGGCTACGGAATTATTGATTATG
AGACACGCGATGTGATTGACGCCGGGGTTAGGCTGTTCAAAGAGGCCAACGTTGAAAACAACGA
GGGAAGACGGAGTAAGCGCGGAGCAAGAAGACTCAAGCGCAGACGGAGACATCGGATTCAGAGG
GTGAAAAAGCTGCTCTTCGATTACAATCTCCTGACCGATCATAGTGAGCTGAGCGGAATCAACC
CCTACGAGGCGCGAGTGAAAGGGCTTTCCCAGAAGCTGTCCGAAGAGGAGTTCTCCGCCGCGTT
GCTGCACCTGGCCAAACGGAGGGGGGTTCACAATGTAAACGAAGTGGAGGAGGACACGGGCAAT
GAACTTAGTACGAAAGAACAGATCAGTAGGAACTCTAAGGCTCTCGAAGAGAAATACGTCGCTG
AGTTGCAGCTTGAGAGACTGAAAAAAGACGGCGAAGTACGCGGATCTATTAATAGGTTCAAGAC
TTCAGATTACGTAAAGGAAGCCAAGCAGCTCCTGAAAGTACAGAAAGCGTACCATCAGCTCGAT
CAGAGCTTCATCGATACCTACATAGATTTGCTGGAGACACGGAGGACATACTACGAGGGCCCAG
GGGAAGGATCTCCTTTTGGGTGGAAGGACATCAAGGAATGGTACGAGATGCTTATGGGACATTG
TACATATTTTCCGGAGGAGCTCAGGAGCGTCAAGTACGCCTACAATGCCGACCTGTACAATGCC
CTCAATGACCTCAATAACCTCGTGATTACCAGGGACGAGAACGAGAAGCTGGAGTACTATGAAA
AGTTCCAGATTATCGAGAATGTGTTTAAGCAGAAGAAGAAGCCGACACTTAAGCAGATTGCAAA
GGAAATCCTCGTGAATGAGGAAGATATCAAGGGATACAGAGTGACAAGTACAGGCAAGCCCGAG
TTCACAAATCTGAAGGTGTACCACGATATTAAGGACATAACCGCACGAAAGGAGATAATCGAAA
ACGCTGAGCTCCTCGATCAGATCGCAAAAATTCTTACCATCTACCAGTCTAGTGAGGACATTCA
GGAGGAACTGACTAATCTGAACAGTGAGCTCACCCAAGAGGAAATTGAGCAGATTTCAAACCTG
AAAGGCTACACCGGGACGCACAATCTGAGCCTCAAAGCAATCAACCTCATTCTGGATGAACTTT
GGCACACAAATGACAACCAAATTGCCATATTCAACCGCCTGAAACTGGTGCCAAAAAAAGTGGA
TCTGTCACAGCAAAAGGAAATCCCTACAACCTTGGTTGACGATTTTATTCTGTCCCCCGTTGTC
AAGCGGAGCTTCATCCAGTCAATCAAGGTGATCAATGCCATCATTAAAAAATACGGATTGCCAA
ACGATATAATTATCGAGCTTGCACGAGAGAAGAACTCAAAGGACGCCCAGAAGATGATTAACGA
AATGCAGAAGCGCAACCGCCAGACAAACGAACGCATAGAGGAAATTATAAGAACAACCGGCAAA
GAGAATGCCAAGTATCTGATCGAGAAAATCAAGCTGCACGACATGCAAGAAGGCAAGTGCCTGT
ACTCTCTGGAAGCTATCCCACTCGAAGATCTGCTGAATAATCCATTCAATTACGAGGTGGACCA
CATCATCCCTAGATCCGTAAGCTTTGACAATTCCTTCAATAACAAAGTTCTGGTTAAACAGGAG
GAAAATTCTAAAAAAGGGAACCGGACCCCGTTCCAGTACCTGAGCTCCAGTGACAGCAAGATTA
GCTACGAGACTTTTAAGAAACATATTCTGAATCTGGCCAAAGGCAAAGGCAGGATCAGCAAGAC
CAAGAAGGAGTACCTCCTCGAAGAACGCGACATTAACAGATTTAGTGTGCAGAAAGATTTCATC
AACCGAAACCTTGTCGATACTCGGTACGCCACGAGAGGCCTGATGAATCTCCTCAGGAGCTACT
TCCGCGTCAATAATCTGGACGTTAAAGTCAAGAGCATAAATGGGGGATTCACCAGCTTTCTGAG
GAGAAAGTGGAAGTTTAAGAAGGAACGAAACAAAGGATACAAGCACCATGCTGAGGATGCTTTG
ATCATCGCTAACGCGGACTTTATCTTTAAGGAATGGAAAAAGCTGGATAAGGCAAAGAAAGTGA
TGGAAAACCAGATGTTCGAGGAGAAGCAGGCAGAGTCAATGCCTGAGATCGAGACAGAGCAGGA
ATACAAGGAAATTTTCATCACCCCTCATCAGATTAAACACATAAAGGACTTCAAAGACTATAAA
TACTCTCATAGGGTGGACAAAAAACCCAATCGCAAGCTCATTAATGACACCCTGTACTCAACAC
GGAAGGATGATAAAGGTAATACCTTGATTGTGAATAATCTTAATGGATTGTATGACAAAGATAA
CGACAAGCTCAAGAAGCTGATCAACAAGTCTCCAGAGAAGCTCCTTATGTATCACCACGACCCA
CAGACTTATCAGAAATTGAAACTGATCATGGAGCAATACGGGGATGAGAAGAACCCACTCTACA
AATATTATGAGGAAACAGGTAATTACCTGACCAAGTACTCCAAGAAGGATAACGGACCAGTGAT
CAAAAAGATAAAGTACTATGGCAACAAACTTAATGCGCATTTGGACATAACTGACGATTACCCC
AATTCTCGAAACAAGGTTGTGAAGCTCTCCCTGAAGCCTTATAGATTTGACGTGTACCTGGATA
ATGGGGTTTATAAATTCGTCACCGTGAAAAATCTGGACGTGATCAAAAAGGAGAACTATTATGA
AGTAAACTCAAAGTGCTATGAGGAGGCGAAGAAGCTGAAGAAGATCTCCAATCAGGCCGAGTTC
ATCGCTTCCTTCTATAAGAACGATCTCATCAAGATCAATGGAGAGCTTTATCGCGTCATTGGTG
TGAACAATGACTTGCTGAACAGGATCGAAGTCAATATGATAGACATTACCTACCGGGAGTATCT
CGAAAACATGAATGATAAACGGCCGCCTCACATCATCAAGACAATCGCATCTAAAACTCAGTCA
ATAAAAAAGTACTCTACCGATATCCTGGGGAATCTCTATGAAGTGAAGTCAAAGAAGCACCCAC
AAATCATTAAAAAAGGTGGATCCCCCAAGAAGAAGAGGAAAGTCTCGAGCGACTACAAAGACCA
TGACGGTGATTATAAAGATCATGACATCGATTACAAGGATGACGATGACAAGTCTGGTGGTTCT

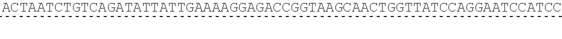

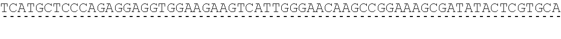

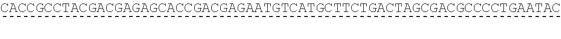

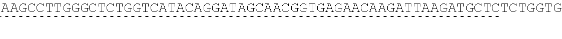

GTTCTCCCAAGAAGAAGAGGAAAGTC
Translated amino acid sequence:
(SEQ ID NO: 290)
PTPYPNYANAGHVEGQSALFMRDNGISEGLVFHNNPEGTCGFCVNMTETLLPENAKMTVVPPEG
AIPVKRGATGETKVFTGNSNSPKSPTKGGCSGGSSGGSSGSETPGTSESATPESSGGSSGGSGK
RNYILGLAIGITSVGYGIIDYETRDVIDAGVRLFKEANVENNEGRRSKRGARRLKRRRRHRIQR
VKKLLFDYNLLTDHSELSGINPYEARVKGLSQKLSEEEFSAALLHLAKRRGVHNVNEVEEDTGN
ELSTKEQISRNSKALEEKYVAELQLERLKKDGEVRGSINRFKTSDYVKEAKOLLKVOKAYHOLD
QSFIDTYIDLLETRRTYYEGPGEGSPFGWKDIKEWYEMLMGHCTYFPEELRSVKYAYNADLYNA
LNDLNNLVITRDENEKLEYYEKFQIIENVFKQKKKPTLKQIAKEILVNEEDIKGYRVTSTGKPE
FTNLKVYHDIKDITARKEIIENAELLDQIAKILTIYQSSEDIQEELTNLNSELTQEEIEQISNL
KGYTGTHNLSLKAINLILDELWHTNDNOIAIFNRLKLVPKKVDLSQQKEIPTTLVDDFILSPVV
KRSFIQSIKVINAIIKKYGLPNDIIIELAREKNSKDAQKMINEMQKRNRQTNERIEEIIRTTGK
ENAKYLIEKIKLHDMQEGKCLYSLEAIPLEDLLNNPFNYEVDHIIPRSVSFDNSFNNKVLVKQE
ENSKKGNRTPFQYLSSSDSKISYETFKKHILNLAKGKGRISKTKKEYLLEERDINRFSVQKDFI
NRNLVDTRYATRGLMNLLRSYFRVNNLDVKVKSINGGFTSFLRRKWKFKKERNKGYKHHAEDAL
IIANADFIFKEWKKLDKAKKVMENOMFEEKQAESMPEIETEQEYKEIFITPHQIKHIKDFKDYK
YSHRVDKKPNRKLINDTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYYGNKLNAHLDITDDYP
NSRNKVVKLSLKPYRFDVYLDNGVYKFVTVKNLDVIKKENYYEVNSKCYEEAKKLKKISNQAEF
IASFYKNDLIKINGELYRVIGVNNDLLNRIEVNMIDITYREYLENMNDKRPPHIIKTIASKTQS
IKKYSTDILGNLYEVKSKKHPQIIKKGGSPKKKRKVSSDYKDHDGDYKDHDIDYKDDDDKSGGS
TNLSDIIEKETGKOLVIQESILMLPEEVEEVIGNKPESDILVHTAYDESTDENVMLLTSDAPEY
KPWALVIQDSNGENKIKMLSGGSPKKKRKV
G1333 DddAₜₒₓ-N-dSpCas9-2x-UGI
(SEQ ID NO: 291)
AAACGGACAGCCGACGGAAGCGAGTTCGAGTCACCAAAGAAGAAGCGGAAAGTCGGCAGCTACG
CCCTGGGTCCGTATCAGATTAGCGCCCCGCAGCTGCCAGCTTACAATGGTCAGACCGTGGGTAC
CTTCTACTATGTGAACGACGCGGGCGGTCTGGAGAGCAAGGTGTTTAGCAGCGGCGGTTCTGGA
GGATCTAGCGGAGGATCCTCTGGCAGCGAGACACCAGGAACAAGCGAGTCAGCAACACCAGAGA
GCAGTGGCGGCAGCAGCGGCGGCAGCGACAAGAAGTACAGCATCGGCCTGGCCATCGGCACCAA
CTCTGTGGGCTGGGCCGTGATCACCGACGAGTACAAGGTGCCCAGCAAGAAATTCAAGGTGCTG
GGCAACACCGACCGGCACAGCATCAAGAAGAACCTGATCGGAGCCCTGCTGTTCGACAGCGGCG
AAACAGCCGAGGCCACCCGGCTGAAGAGAACCGCCAGAAGAAGATACACCAGACGGAAGAACCG
GATCTGCTATCTGCAAGAGATCTTCAGCAACGAGATGGCCAAGGTGGACGACAGCTTCTTCCAC
AGACTGGAAGAGTCCTTCCTGGTGGAAGAGGATAAGAAGCACGAGCGGCACCCCATCTTCGGCA
ACATCGTGGACGAGGTGGCCTACCACGAGAAGTACCCCACCATCTACCACCTGAGAAAGAAACT
GGTGGACAGCACCGACAAGGCCGACCTGCGGCTGATCTATCTGGCCCTGGCCCACATGATCAAG
TTCCGGGGCCACTTCCTGATCGAGGGCGACCTGAACCCCGACAACAGCGACGTGGACAAGCTGT
TCATCCAGCTGGTGCAGACCTACAACCAGCTGTTCGAGGAAAACCCCATCAACGCCAGCGGCGT
GGACGCCAAGGCCATCCTGTCTGCCAGACTGAGCAAGAGCAGACGGCTGGAAAATCTGATCGCC
CAGCTGCCCGGCGAGAAGAAGAATGGCCTGTTCGGAAACCTGATTGCCCTGAGCCTGGGCCTGA
CCCCCAACTTCAAGAGCAACTTCGACCTGGCCGAGGATGCCAAACTGCAGCTGAGCAAGGACAC
CTACGACGACGACCTGGACAACCTGCTGGCCCAGATCGGCGACCAGTACGCCGACCTGTTTCTG
GCCGCCAAGAACCTGTCCGACGCCATCCTGCTGAGCGACATCCTGAGAGTGAACACCGAGATCA
CCAAGGCCCCCCTGAGCGCCTCTATGATCAAGAGATACGACGAGCACCACCAGGACCTGACCCT
GCTGAAAGCTCTCGTGCGGCAGCAGCTGCCTGAGAAGTACAAAGAGATTTTCTTCGACCAGAGC
AAGAACGGCTACGCCGGCTACATTGACGGCGGAGCCAGCCAGGAAGAGTTCTACAAGTTCATCA
AGCCCATCCTGGAAAAGATGGACGGCACCGAGGAACTGCTCGTGAAGCTGAACAGAGAGGACCT
GCTGCGGAAGCAGCGGACCTTCGACAACGGCAGCATCCCCCACCAGATCCACCTGGGAGAGCTG
CACGCCATTCTGCGGCGGCAGGAAGATTTTTACCCATTCCTGAAGGACAACCGGGAAAAGATCG
AGAAGATCCTGACCTTCCGCATCCCCTACTACGTGGGCCCTCTGGCCAGGGGAAACAGCAGATT
CGCCTGGATGACCAGAAAGAGCGAGGAAACCATCACCCCCTGGAACTTCGAGGAAGTGGTGGAC
AAGGGCGCTTCCGCCCAGAGCTTCATCGAGCGGATGACCAACTTCGATAAGAACCTGCCCAACG
AGAAGGTGCTGCCCAAGCACAGCCTGCTGTACGAGTACTTCACCGTGTATAACGAGCTGACCAA
AGTGAAATACGTGACCGAGGGAATGAGAAAGCCCGCCTTCCTGAGCGGCGAGCAGAAAAAGGCC
ATCGTGGACCTGCTGTTCAAGACCAACCGGAAAGTGACCGTGAAGCAGCTGAAAGAGGACTACT
TCAAGAAAATCGAGTGCTTCGACTCCGTGGAAATCTCCGGCGTGGAAGATCGGTTCAACGCCTC
CCTGGGCACATACCACGATCTGCTGAAAATTATCAAGGACAAGGACTTCCTGGACAATGAGGAA
AACGAGGACATTCTGGAAGATATCGTGCTGACCCTGACACTGTTTGAGGACAGAGAGATGATCG
AGGAACGGCTGAAAACCTATGCCCACCTGTTCGACGACAAAGTGATGAAGCAGCTGAAGCGGCG
GAGATACACCGGCTGGGGCAGGCTGAGCCGGAAGCTGATCAACGGCATCCGGGACAAGCAGTCC
GGCAAGACAATCCTGGATTTCCTGAAGTCCGACGGCTTCGCCAACAGAAACTTCATGCAGCTGA
TCCACGACGACAGCCTGACCTTTAAAGAGGACATCCAGAAAGCCCAGGTGTCCGGCCAGGGCGA
TAGCCTGCACGAGCACATTGCCAATCTGGCCGGCAGCCCCGCCATTAAGAAGGGCATCCTGCAG
ACAGTGAAGGTGGTGGACGAGCTCGTGAAAGTGATGGGCCGGCACAAGCCCGAGAACATCGTGA
TCGAAATGGCCAGAGAGAACCAGACCACCCAGAAGGGACAGAAGAACAGCCGCGAGAGAATGAA
GCGGATCGAAGAGGGCATCAAAGAGCTGGGCAGCCAGATCCTGAAAGAACACCCCGTGGAAAAC
ACCCAGCTGCAGAACGAGAAGCTGTACCTGTACTACCTGCAGAATGGGCGGGATATGTACGTGG
ACCAGGAACTGGACATCAACCGGCTGTCCGACTACGATGTGGACGCTATCGTGCCTCAGAGCTT
TCTGAAGGACGACTCCATCGACAACAAGGTGCTGACCAGAAGCGACAAGAACCGGGGCAAGAGC
GACAACGTGCCCTCCGAAGAGGTCGTGAAGAAGATGAAGAACTACTGGCGGCAGCTGCTGAACG
CCAAGCTGATTACCCAGAGAAAGTTCGACAATCTGACCAAGGCCGAGAGAGGCGGCCTGAGCGA
ACTGGATAAGGCCGGCTTCATCAAGAGACAGCTGGTGGAAACCCGGCAGATCACAAAGCACGTG
GCACAGATCCTGGACTCCCGGATGAACACTAAGTACGACGAGAATGACAAGCTGATCCGGGAAG
TGAAAGTGATCACCCTGAAGTCCAAGCTGGTGTCCGATTTCCGGAAGGATTTCCAGTTTTACAA
AGTGCGCGAGATCAACAACTACCACCACGCCCACGACGCCTACCTGAACGCCGTCGTGGGAACC
GCCCTGATCAAAAAGTACCCTAAGCTGGAAAGCGAGTTCGTGTACGGCGACTACAAGGTGTACG
ACGTGCGGAAGATGATCGCCAAGAGCGAGCAGGAAATCGGCAAGGCTACCGCCAAGTACTTCTT
CTACAGCAACATCATGAACTTTTTCAAGACCGAGATTACCCTGGCCAACGGCGAGATCCGGAAG
CGGCCTCTGATCGAGACAAACGGCGAAACCGGGGAGATCGTGTGGGATAAGGGCCGGGATTTTG
CCACCGTGCGGAAAGTGCTGAGCATGCCCCAAGTGAATATCGTGAAAAAGACCGAGGTGCAGAC
AGGCGGCTTCAGCAAAGAGTCTATCCTGCCCAAGAGGAACAGCGATAAGCTGATCGCCAGAAAG
AAGGACTGGGACCCTAAGAAGTACGGCGGCTTCGACAGCCCCACCGTGGCCTATTCTGTGCTGG
TGGTGGCCAAAGTGGAAAAGGGCAAGTCCAAGAAACTGAAGAGTGTGAAAGAGCTGCTGGGGAT
CACCATCATGGAAAGAAGCAGCTTCGAGAAGAATCCCATCGACTTTCTGGAAGCCAAGGGCTAC
AAAGAAGTGAAAAAGGACCTGATCATCAAGCTGCCTAAGTACTCCCTGTTCGAGCTGGAAAACG
GCCGGAAGAGAATGCTGGCCTCTGCCGGCGAACTGCAGAAGGGAAACGAACTGGCCCTGCCCTC
CAAATATGTGAACTTCCTGTACCTGGCCAGCCACTATGAGAAGCTGAAGGGCTCCCCCGAGGAT
AATGAGCAGAAACAGCTGTTTGTGGAACAGCACAAGCACTACCTGGACGAGATCATCGAGCAGA
TCAGCGAGTTCTCCAAGAGAGTGATCCTGGCCGACGCTAATCTGGACAAAGTGCTGTCCGCCTA
CAACAAGCACCGGGATAAGCCCATCAGAGAGCAGGCCGAGAATATCATCCACCTGTTTACCCTG
ACCAATCTGGGAGCCCCTGCCGCCTTCAAGTACTTTGACACCACCATCGACCGGAAGAGGTACA
CCAGCACCAAAGAGGTGCTGGACGCCACCCTGATCCACCAGAGCATCACCGGCCTGTACGAGAC

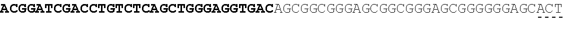

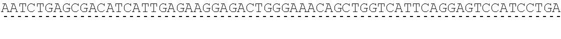

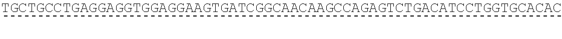

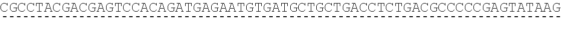

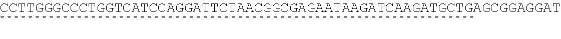

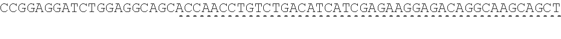

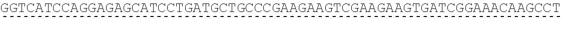

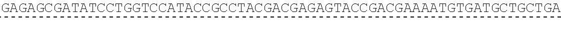

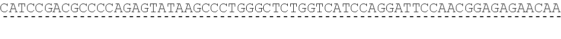

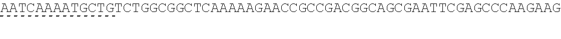

AAGAGGAAAGTC
Translated amino acid sequence:
(SEQ ID NO: 292)
KRTADGSEFESPKKKRKVGSYALGPYQISAPOLPAYNGQTVGTFYYVNDAGGLESKVFSSGGSG
GSSGGSSGSETPGTSESATPESSGGSSGGSDKKYSIGLAIGTNSVGWAVITDEYKVPSKKFKVL
GNTDRHSIKKNLIGALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRRLENLIA
QLPGEKKNGLFGNLIALSLGLTPNFKSNFDLAEDAKLQLSKDTYDDDLDNLLAQIGDQYADLFL
AAKNLSDAILLSDILRVNTEITKAPLSASMIKRYDEHHODLTLLKALVROOLPEKYKEIFFDQS
KNGYAGYIDGGASQEEFYKFIKPILEKMDGTEELLVKLNREDLLRKORTFDNGSIPHQIHLGEL
HAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKA
IVDLLFKTNRKVTVKOLKEDYFKKIECFDSVEISGVEDRFNASLGTYHDLLKIIKDKDFLDNEE
NEDILEDIVLTLTLFEDREMIEERLKTYAHLFDDKVMKOLKRRRYTGWGRLSRKLINGIRDKOS
GKTILDFLKSDGFANRNFMOLIHDDSLTFKEDIQKAQVSGQGDSLHEHIANLAGSPAIKKGILQ
TVKVVDELVKVMGRHKPENIVIEMARENOTTOKGOKNSRERMKRIEEGIKELGSQILKEHPVEN
TQLQNEKLYLYYLONGRDMYVDQELDINRLSDYDVDAIVPQSFLKDDSIDNKVLTRSDKNRGKS
DNVPSEEVVKKMKNYWROLLNAKLITORKFDNLTKAERGGLSELDKAGFIKROLVETROITKHV
AQILDSRMNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPIDFLEAKGY
KEVKKDLIIKLPKYSLFELENGRKRMLASAGELQKGNELALPSKYVNFLYLASHYEKLKGSPED
NEQKOLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSGGSGGST
NLSDIIEKETGKOLVIQESILMLPEEVEEVIGNKPESDILVHTAYDESTDENVMLLTSDAPEYK
PWALVIQDSNGENKIKMLSGGSGGSGGSTNLSDIIEKETGKOLVIQESILMLPEEVEEVIGNKP
ESDILVHTAYDESTDENVMLLTSDAPEYKPWALVIQDSNGENKIKMLSGGSKRTADGSEFEPKK
KRKV
G1333 DddAₜₒₓ-C-dSpCas9-2x-UGI
(SEQ ID NO: 293)
AAACGGACAGCCGACGGAAGCGAGTTCGAGTCACCAAAGAAGAAGCGGAAAGTCCCAACCCCGT
ACCCAAACTATGCCAATGCCGGTCATGTGGAGGGTCAGAGCGCCCTGTTCATGCGTGATAACGG
CATCAGCGAGGGTCTGGTGTTCCACAACAACCCGGAAGGCACCTGCGGTTTTTGCGTGAACATG
ACCGAGACCCTGCTGCCGGAAAACGCGAAAATGACCGTGGTGCCGCCGGAAGGTGCCATTCCAG
TGAAGCGCGGCGCTACCGGTGAAACCAAAGTGTTTACCGGTAACAGCAACAGCCCGAAGAGCCC
GACCAAAGGCGGTTGCTTCTGGAGGATCTAGCGGAGGATCCTCTGGCAGCGAGACACCAGGAAC
AAGCGAGTCAGCAACACCAGAGAGCAGTGGCGGCAGCAGCGGCGGCAGCGACAAGAAGTACAGC
ATCGGCCTGGCCATCGGCACCAACTCTGTGGGCTGGGCCGTGATCACCGACGAGTACAAGGTGC
CCAGCAAGAAATTCAAGGTGCTGGGCAACACCGACCGGCACAGCATCAAGAAGAACCTGATCGG
AGCCCTGCTGTTCGACAGCGGCGAAACAGCCGAGGCCACCCGGCTGAAGAGAACCGCCAGAAGA
AGATACACCAGACGGAAGAACCGGATCTGCTATCTGCAAGAGATCTTCAGCAACGAGATGGCCA
AGGTGGACGACAGCTTCTTCCACAGACTGGAAGAGTCCTTCCTGGTGGAAGAGGATAAGAAGCA
CGAGCGGCACCCCATCTTCGGCAACATCGTGGACGAGGTGGCCTACCACGAGAAGTACCCCACC
ATCTACCACCTGAGAAAGAAACTGGTGGACAGCACCGACAAGGCCGACCTGCGGCTGATCTATC
TGGCCCTGGCCCACATGATCAAGTTCCGGGGCCACTTCCTGATCGAGGGCGACCTGAACCCCGA
CAACAGCGACGTGGACAAGCTGTTCATCCAGCTGGTGCAGACCTACAACCAGCTGTTCGAGGAA
AACCCCATCAACGCCAGCGGCGTGGACGCCAAGGCCATCCTGTCTGCCAGACTGAGCAAGAGCA
GACGGCTGGAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCGAGGATGCC
AAACTGCAGCTGAGCAAGGACACCTACGACGACGACCTGGACAACCTGCTGGCCCAGATCGGCG
ACCAGTACGCCGACCTGTTTCTGGCCGCCAAGAACCTGTCCGACGCCATCCTGCTGAGCGACAT
CCTGAGAGTGAACACCGAGATCACCAAGGCCCCCCTGAGCGCCTCTATGATCAAGAGATACGAC
GAGCACCACCAGGACCTGACCCTGCTGAAAGCTCTCGTGCGGCAGCAGCTGCCTGAGAAGTACA
AAGAGATTTTCTTCGACCAGAGCAAGAACGGCTACGCCGGCTACATTGACGGCGGAGCCAGCCA
GGAAGAGTTCTACAAGTTCATCAAGCCCATCCTGGAAAAGATGGACGGCACCGAGGAACTGCTC
GTGAAGCTGAACAGAGAGGACCTGCTGCGGAAGCAGCGGACCTTCGACAACGGCAGCATCCCCC
ACCAGATCCACCTGGGAGAGCTGCACGCCATTCTGCGGCGGCAGGAAGATTTTTACCCATTCCT
GAAGGACAACCGGGAAAAGATCGAGAAGATCCTGACCTTCCGCATCCCCTACTACGTGGGCCCT
CTGGCCAGGGGAAACAGCAGATTCGCCTGGATGACCAGAAAGAGCGAGGAAACCATCACCCCCT
GGAACTTCGAGGAAGTGGTGGACAAGGGCGCTTCCGCCCAGAGCTTCATCGAGCGGATGACCAA
CTTCGATAAGAACCTGCCCAACGAGAAGGTGCTGCCCAAGCACAGCCTGCTGTACGAGTACTTC
ACCGTGTATAACGAGCTGACCAAAGTGAAATACGTGACCGAGGGAATGAGAAAGCCCGCCTTCC
TGAGCGGCGAGCAGAAAAAGGCCATCGTGGACCTGCTGTTCAAGACCAACCGGAAAGTGACCGT
GAAGCAGCTGAAAGAGGACTACTTCAAGAAAATCGAGTGCTTCGACTCCGTGGAAATCTCCGGC
GTGGAAGATCGGTTCAACGCCTCCCTGGGCACATACCACGATCTGCTGAAAATTATCAAGGACA
AGGACTTCCTGGACAATGAGGAAAACGAGGACATTCTGGAAGATATCGTGCTGACCCTGACACT
GTTTGAGGACAGAGAGATGATCGAGGAACGGCTGAAAACCTATGCCCACCTGTTCGACGACAAA
GTGATGAAGCAGCTGAAGCGGCGGAGATACACCGGCTGGGGCAGGCTGAGCCGGAAGCTGATCA
ACGGCATCCGGGACAAGCAGTCCGGCAAGACAATCCTGGATTTCCTGAAGTCCGACGGCTTCGC
CAACAGAAACTTCATGCAGCTGATCCACGACGACAGCCTGACCTTTAAAGAGGACATCCAGAAA
GCCCAGGTGTCCGGCCAGGGCGATAGCCTGCACGAGCACATTGCCAATCTGGCCGGCAGCCCCG
CCATTAAGAAGGGCATCCTGCAGACAGTGAAGGTGGTGGACGAGCTCGTGAAAGTGATGGGCCG
GCACAAGCCCGAGAACATCGTGATCGAAATGGCCAGAGAGAACCAGACCACCCAGAAGGGACAG
AAGAACAGCCGCGAGAGAATGAAGCGGATCGAAGAGGGCATCAAAGAGCTGGGCAGCCAGATCC
TGAAAGAACACCCCGTGGAAAACACCCAGCTGCAGAACGAGAAGCTGTACCTGTACTACCTGCA
GAATGGGCGGGATATGTACGTGGACCAGGAACTGGACATCAACCGGCTGTCCGACTACGATGTG
GACGCTATCGTGCCTCAGAGCTTTCTGAAGGACGACTCCATCGACAACAAGGTGCTGACCAGAA
GCGACAAGAACCGGGGCAAGAGCGACAACGTGCCCTCCGAAGAGGTCGTGAAGAAGATGAAGAA
CTACTGGCGGCAGCTGCTGAACGCCAAGCTGATTACCCAGAGAAAGTTCGACAATCTGACCAAG
GCCGAGAGAGGCGGCCTGAGCGAACTGGATAAGGCCGGCTTCATCAAGAGACAGCTGGTGGAAA
CCCGGCAGATCACAAAGCACGTGGCACAGATCCTGGACTCCCGGATGAACACTAAGTACGACGA
GAATGACAAGCTGATCCGGGAAGTGAAAGTGATCACCCTGAAGTCCAAGCTGGTGTCCGATTTC
CGGAAGGATTTCCAGTTTTACAAAGTGCGCGAGATCAACAACTACCACCACGCCCACGACGCCT
ACCTGAACGCCGTCGTGGGAACCGCCCTGATCAAAAAGTACCCTAAGCTGGAAAGCGAGTTCGT
GTACGGCGACTACAAGGTGTACGACGTGCGGAAGATGATCGCCAAGAGCGAGCAGGAAATCGGC
AAGGCTACCGCCAAGTACTTCTTCTACAGCAACATCATGAACTTTTTCAAGACCGAGATTACCC
TGGCCAACGGCGAGATCCGGAAGCGGCCTCTGATCGAGACAAACGGCGAAACCGGGGAGATCGT
GTGGGATAAGGGCCGGGATTTTGCCACCGTGCGGAAAGTGCTGAGCATGCCCCAAGTGAATATC
GTGAAAAAGACCGAGGTGCAGACAGGCGGCTTCAGCAAAGAGTCTATCCTGCCCAAGAGGAACA
GCGATAAGCTGATCGCCAGAAAGAAGGACTGGGACCCTAAGAAGTACGGCGGCTTCGACAGCCC
CACCGTGGCCTATTCTGTGCTGGTGGTGGCCAAAGTGGAAAAGGGCAAGTCCAAGAAACTGAAG
AGTGTGAAAGAGCTGCTGGGGATCACCATCATGGAAAGAAGCAGCTTCGAGAAGAATCCCATCG
ACTTTCTGGAAGCCAAGGGCTACAAAGAAGTGAAAAAGGACCTGATCATCAAGCTGCCTAAGTA
CTCCCTGTTCGAGCTGGAAAACGGCCGGAAGAGAATGCTGGCCTCTGCCGGCGAACTGCAGAAG
GGAAACGAACTGGCCCTGCCCTCCAAATATGTGAACTTCCTGTACCTGGCCAGCCACTATGAGA
AGCTGAAGGGCTCCCCCGAGGATAATGAGCAGAAACAGCTGTTTGTGGAACAGCACAAGCACTA
CCTGGACGAGATCATCGAGCAGATCAGCGAGTTCTCCAAGAGAGTGATCCTGGCCGACGCTAAT
CTGGACAAAGTGCTGTCCGCCTACAACAAGCACCGGGATAAGCCCATCAGAGAGCAGGCCGAGA
ATATCATCCACCTGTTTACCCTGACCAATCTGGGAGCCCCTGCCGCCTTCAAGTACTTTGACAC
CACCATCGACCGGAAGAGGTACACCAGCACCAAAGAGGTGCTGGACGCCACCCTGATCCACCAG
AGCATCACCGGCCTGTACGAGACACGGATCGACCTGTCTCAGCTGGGAGGTGACAGCGGCGGGA

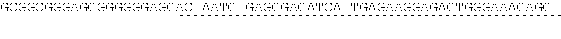

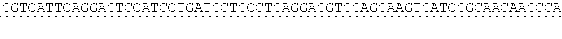

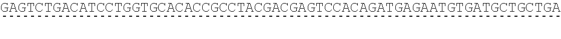

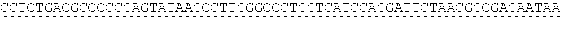

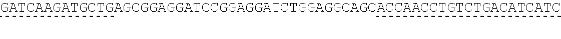

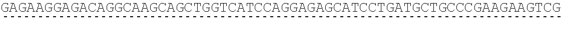

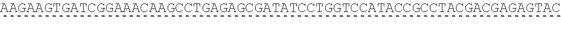

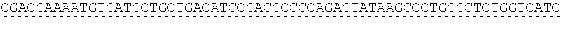

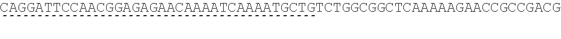

GCAGCGAATTCGAGCCCAAGAAGAAGAGGAAAGTC

### Split-DddAₜₒₓ-CCR5 TALE Fusions Sequences

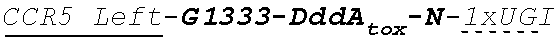

(SEQ ID NO: 294)
CCCAAGAAGAAGAGGAAGGTGGGCATTCACCGCGGGGTACCTATGGTGGACTTGAGGACACTCG
GTTATTCGCAACAGCAACAGGAGAAAATCAAGCCTAAGGTCAGGAGCACCGTCGCGCAACACCA
CGAGGCGCTTGTGGGGCATGGCTTCACTCATGCGCATATTGTCGCGCTTTCACAGCACCCTGCG
GCGCTTGGGACGGTGGCTGTCAAATACCAAGATATGATTGCGGCCCTGCCCGAAGCCACGCACG
AGGCAATTGTAGGGGTCGGTAAACAGTGGTCGGGAGCGCGAGCACTTGAGGCGCTGCTGACTGT
GGCGGGTGAGCTTAGGGGGCCTCCGCTCCAGCTCGACACCGGGCAGCTGCTGAAGATCGCGAAG
AGAGGGGGAGTAACAGCGGTAGAGGCAGTGCACGCCTGGCGCAATGCGCTCACCGGGGCCCCCT
TGAACCTGACCCCAGACCAGGTAGTCGCAATCGCGTCAAACGGAGGGGGAAAGCAAGCCCTGGA
AACCGTGCAAAGGTTGTTGCCGGTCCTTTGTCAAGACCACGGCCTTACACCGGAGCAAGTCGTG
GCCATTGCATCCCACGACGGTGGCAAACAGGCTCTTGAGACGGTTCAGAGACTTCTCCCAGTTC
TCTGTCAAGCCCACGGGCTGACTCCCGATCAAGTTGTAGCGATTGCGTCGAACATTGGAGGGAA
ACAAGCATTGGAGACTGTCCAACGGCTCCTTCCCGTGTTGTGTCAAGCCCACGGTTTGACGCCT
GCACAAGTGGTCGCCATCGCCTCGAATGGCGGCGGTAAGCAGGCGCTGGAAACAGTACAGCGCC
TGCTGCCTGTACTGTGCCAGGATCATGGACTGACCCCAGACCAGGTAGTCGCAATCGCGTCAAA
CGGAGGGGGAAAGCAAGCCCTGGAAACCGTGCAAAGGTTGTTGCCGGTCCTTTGTCAAGACCAC
GGCCTTACACCGGAGCAAGTCGTGGCCATTGCAAGCAACATCGGTGGCAAACAGGCTCTTGAGA
CGGTTCAGAGACTTCTCCCAGTTCTCTGTCAAGCCCACGGGCTGACTCCCGATCAAGTTGTAGC
GATTGCGTCGCATGACGGAGGGAAACAAGCATTGGAGACTGTCCAACGGCTCCTTCCCGTGTTG
TGTCAAGCCCACGGTTTGACGCCTGCACAAGTGGTCGCCATCGCCTCCAATATTGGCGGTAAGC
AGGCGCTGGAAACAGTACAGCGCCTGCTGCCTGTACTGTGCCAGGATCATGGACTGACCCCAGA
CCAGGTAGTCGCAATCGCGTCACATGACGGGGGAAAGCAAGCCCTGGAAACCGTGCAAAGGTTG
TTGCCGGTCCTTTGTCAAGACCACGGCCTTACACCGGAGCAAGTCGTGGCCATTGCATCCCACG
ACGGTGGCAAACAGGCTCTTGAGACGGTTCAGAGACTTCTCCCAGTTCTCTGTCAAGCCCACGG
GCTGACTCCCGATCAAGTTGTAGCGATTGCGTCCAACGGTGGAGGGAAACAAGCATTGGAGACT
GTCCAACGGCTCCTTCCCGTGTTGTGTCAAGCCCACGGTTTGACGCCTGCACAAGTGGTCGCCA
TCGCCAACAACAACGGCGGTAAGCAGGCGCTGGAAACAGTACAGCGCCTGCTGCCTGTACTGTG
CCAGGATCATGGACTGACCCCAGACCAGGTAGTCGCAATCGCGTCACATGACGGGGGAAAGCAA
GCCCTGGAAACCGTGCAAAGGTTGTTGCCGGTCCTTTGTCAAGACCACGGCCTTACACCGGAGC
AAGTCGTGGCCATTGCAAGCAACATCGGTGGCAAACAGGCTCTTGAGACGGTTCAGAGACTTCT
CCCAGTTCTCTGTCAAGCCCACGGGCTGACTCCCGATCAAGTTGTAGCGATTGCGAATAACAAT
GGAGGGAAACAAGCATTGGAGACTGTCCAACGGCTCCTTCCCGTGTTGTGTCAAGCCCACGGTT
TGACGCCTGCACAAGTGGTCGCCATCGCCAGCCATGATGGCGGTAAGCAGGCGCTGGAAACAGT
ACAGCGCCTGCTGCCTGTACTGTGCCAGGATCATGGACTGACACCCGAACAGGTGGTCGCCATT
GCTTCTAATGGGGGAGGACGGCCAGCCTTGGAGTCCATCGTAGCCCAATTGTCCAGGCCCGATC
CCGCGTTGGCTGCGTTAACGAATGACCATCTGGTGGCGTTGGCATGTCTTGGTGGACGACCCGC
GCTCGATGCAGTCAAAAAGGGTCTGCCTCATGCTCCCGCATTGATCAAAAGAACCAACCGGCGG
ATTCCCGAGAGAACTTCCCATCGAGTCGCGGGATCCGGCAGCTACGCCCTGGGTCCGTATCAGA
TTAGCGCCCCGCAGCTGCCAGCTTACAATGGTCAGACCGTGGGTACCTTCTACTATGTGAACGA

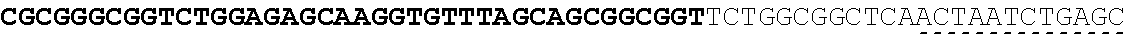

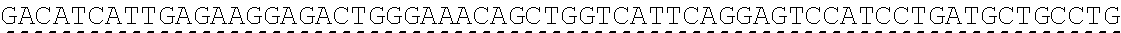

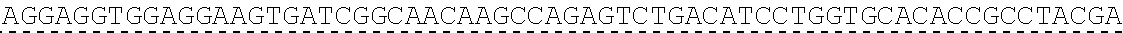

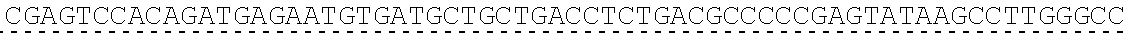

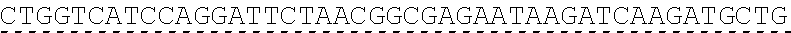

Tranlated amino acid sequence:
(SEQ ID NO: 295)
PKKKRKVGIHRGVPMVDLRTLGYSQQQQEKIKPKVRSTVAQHHEALVGHGFTHAHIVALSQHPA
ALGTVAVKYQDMIAALPEATHEAIVGVGKOWSGARALEALLTVAGELRGPPLOLDTGQLLKIAK
RGGVTAVEAVHAWRNALTGAPLNLTPDQVVAIASNGGGKQALETVORLLPVLCQDHGLTPEQVV
AIASHDGGKQALETVQRLLPVLCQAHGLTPDQVVAIASNIGGKQALETVORLLPVLCQAHGLTP
AQVVAIASNGGGKQALETVORLLPVLCQDHGLTPDQVVAIASNGGGKQALETVORLLPVLCQDH
GLTPEQVVAIASNIGGKQALETVORLLPVLCQAHGLTPDQVVAIASHDGGKQALETVQRLLPVL
CQAHGLTPAQVVAIASNIGGKQALETVORLLPVLCQDHGLTPDQVVAIASHDGGKQALETVORL
LPVLCQDHGLTPEQVVAIASHDGGKQALETVORLLPVLCQAHGLTPDQVVAIASNGGGKQALET
VQRLLPVLCQAHGLTPAQVVAIANNNGGKQALETVORLLPVLCQDHGLTPDQVVAIASHDGGKQ
ALETVQRLLPVLCQDHGLTPEQVVAIASNIGGKQALETVQRLLPVLCQAHGLTPDQVVAIANNN
GGKQALETVORLLPVLCQAHGLTPAQVVAIASHDGGKQALETVORLLPVLCQDHGLTPEQVVAI
ASNGGGRPALESIVAQLSRPDPALAALTNDHLVALACLGGRPALDAVKKGLPHAPALIKRTNRR
IPERTSHRVAGSGSYALGPYQISAPOLPAYNGQTVGTFYYVNDAGGLESKVFSSGGSGGSTNLS
DIIEKETGKQLVIQESILMLPEEVEEVIGNKPESDILVHTAYDESTDENVMLLTSDAPEYKPWA
LVIQDSNGENKIKML

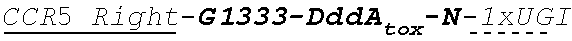

(SEQ ID NO: 296)
CCCAAGAAGAAGAGGAAGGTGGGCATTCACCGCGGGGTACCTATGGTGGACTTGAGGACACTCG
GTTATTCGCAACAGCAACAGGAGAAAATCAAGCCTAAGGTCAGGAGCACCGTCGCGCAACACCA
CGAGGCGCTTGTGGGGCATGGCTTCACTCATGCGCATATTGTCGCGCTTTCACAGCACCCTGCG
GCGCTTGGGACGGTGGCTGTCAAATACCAAGATATGATTGCGGCCCTGCCCGAAGCCACGCACG
AGGCAATTGTAGGGGTCGGTAAACAGTGGTCGGGAGCGCGAGCACTTGAGGCGCTGCTGACTGT
GGCGGGTGAGCTTAGGGGGCCTCCGCTCCAGCTCGACACCGGGCAGCTGCTGAAGATCGCGAAG
AGAGGGGGAGTAACAGCGGTAGAGGCAGTGCACGCCTGGCGCAATGCGCTCACCGGGGCCCCCT
TGAACCTGACCCCAGACCAGGTAGTCGCAATCGCGTCACATGACGGGGGAAAGCAAGCCCTGGA
AACCGTGCAAAGGTTGTTGCCGGTCCTTTGTCAAGACCACGGCCTTACACCGGAGCAAGTCGTG
GCCATTGCAAGCAATGGGGGTGGCAAACAGGCTCTTGAGACGGTTCAGAGACTTCTCCCAGTTC
TCTGTCAAGCCCACGGGCTGACTCCCGATCAAGTTGTAGCGATTGCGTCCAACGGTGGAGGGAA
ACAAGCATTGGAGACTGTCCAACGGCTCCTTCCCGTGTTGTGTCAAGCCCACGGTTTGACGCCT
GCACAAGTGGTCGCCATCGCCAGCCATGATGGCGGTAAGCAGGCGCTGGAAACAGTACAGCGCC
TGCTGCCTGTACTGTGCCAGGATCATGGACTGACCCCAGACCAGGTAGTCGCAATCGCGTCACA
TGACGGGGGAAAGCAAGCCCTGGAAACCGTGCAAAGGTTGTTGCCGGTCCTTTGTCAAGACCAC
GGCCTTACACCGGAGCAAGTCGTGGCCATTGCAAGCAACATCGGTGGCAAACAGGCTCTTGAGA
CGGTTCAGAGACTTCTCCCAGTTCTCTGTCAAGCCCACGGGCTGACTCCCGATCAAGTTGTAGC
GATTGCGAATAACAATGGAGGGAAACAAGCATTGGAGACTGTCCAACGGCTCCTTCCCGTGTTG
TGTCAAGCCCACGGTTTGACGCCTGCACAAGTGGTCGCCATCGCCTCCAATATTGGCGGTAAGC
AGGCGCTGGAAACAGTACAGCGCCTGCTGCCTGTACTGTGCCAGGATCATGGACTGACCCCAGA
CCAGGTAGTCGCAATCGCGTCGAACATTGGGGGAAAGCAAGCCCTGGAAACCGTGCAAAGGTTG
TTGCCGGTCCTTTGTCAAGACCACGGCCTTACACCGGAGCAAGTCGTGGCCATTGCAAGCAATG
GGGGTGGCAAACAGGCTCTTGAGACGGTTCAGAGACTTCTCCCAGTTCTCTGTCAAGCCCACGG
GCTGACTCCCGATCAAGTTGTAGCGATTGCGTCCAACGGTGGAGGGAAACAAGCATTGGAGACT
GTCCAACGGCTCCTTCCCGTGTTGTGTCAAGCCCACGGTTTGACGCCTGCACAAGTGGTCGCCA
TCGCCAACAACAACGGCGGTAAGCAGGCGCTGGAAACAGTACAGCGCCTGCTGCCTGTACTGTG
CCAGGATCATGGACTGACCCCAGACCAGGTAGTCGCAATCGCGTCGAACATTGGGGGAAAGCAA
GCCCTGGAAACCGTGCAAAGGTTGTTGCCGGTCCTTTGTCAAGACCACGGCCTTACACCGGAGC
AAGTCGTGGCCATTGCAAGCAATGGGGGTGGCAAACAGGCTCTTGAGACGGTTCAGAGACTTCT
CCCAGTTCTCTGTCAAGCCCACGGGCTGACTCCCGATCAAGTTGTAGCGATTGCGTCGAACATT
GGAGGGAAACAAGCATTGGAGACTGTCCAACGGCTCCTTCCCGTGTTGTGTCAAGCCCACGGTT
TGACGCCTGCACAAGTGGTCGCCATCGCCAGCCATGATGGCGGTAAGCAGGCGCTGGAAACAGT
ACAGCGCCTGCTGCCTGTACTGTGCCAGGATCATGGACTGACACCCGAACAGGTGGTCGCCATT
GCTTCTAATGGGGGAGGACGGCCAGCCTTGGAGTCCATCGTAGCCCAATTGTCCAGGCCCGATC
CCGCGTTGGCTGCGTTAACGAATGACCATCTGGTGGCGTTGGCATGTCTTGGTGGACGACCCGC
GCTCGATGCAGTCAAAAAGGGTCTGCCTCATGCTCCCGCATTGATCAAAAGAACCAACCGGCGG
ATTCCCGAGAGAACTTCCCATCGAGTCGCGGGATCCCCAACCCCGTACCCAAACTATGCCAATG
CCGGTCATGTGGAGGGTCAGAGCGCCCTGTTCATGCGTGATAACGGCATCAGCGAGGGTCTGGT
GTTCCACAACAACCCGGAAGGCACCTGCGGTTTTTGCGTGAACATGACCGAGACCCTGCTGCCG
GAAAACGCGAAAATGACCGTGGTGCCGCCGGAAGGTGCCATTCCAGTGAAGCGCGGCGCTACCG
GTGAAACCAAAGTGTTTACCGGTAACAGCAACAGCCCGAAGAGCCCGACCAAAGGCGGTTGCTC

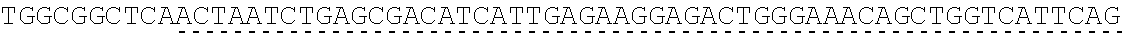

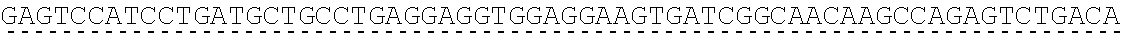

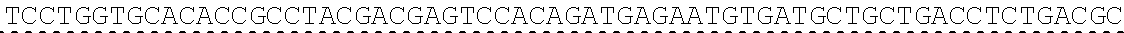

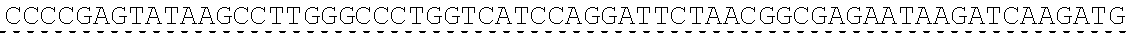

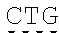

Translated amino acid sequence:
(SEQ ID NO: 297)
PKKKRKVGIHRGVPMVDLRTLGYSQQQQEKIKPKVRSTVAQHHEALVGHGFTHAHIVALSQHPA
ALGTVAVKYQDMIAALPEATHEAIVGVGKOWSGARALEALLTVAGELRGPPLOLDTGQLLKIAK
RGGVTAVEAVHAWRNALTGAPLNLTPDQVVAIASHDGGKQALETVORLLPVLCQDHGLTPEQVV
AIASNGGGKQALETVQRLLPVLCQAHGLTPDQVVAIASNGGGKQALETVORLLPVLCQAHGLTP
AQVVAIASHDGGKQALETVQRLLPVLCQDHGLTPDQVVAIASHDGGKOALETVQRLLPVLCQDH
GLTPEQVVAIASNIGGKQALETVORLLPVLCQAHGLTPDQVVAIANNNGGKQALETVORLLPVL
CQAHGLTPAQVVAIASNIGGKQALETVQRLLPVLCQDHGLTPDQVVAIASNIGGKQALETVQRL
LPVLCQDHGLTPEQVVAIASNGGGKQALETVQRLLPVLCQAHGLTPDQVVAIASNGGGKQALET
VQRLLPVLCQAHGLTPAQVVAIANNNGGKQALETVORLLPVLCQDHGLTPDQVVAIASNIGGKQ
ALETVQRLLPVLCQDHGLTPEQVVAIASNGGGKQALETVORLLPVLCQAHGLTPDQVVAIASNI
GGKQALETVORLLPVLCQAHGLTPAQVVAIASHDGGKQALETVQRLLPVLCQDHGLTPEQVVAI
ASNGGGRPALESIVAQLSRPDPALAALTNDHLVALACLGGRPALDAVKKGLPHAPALIKRINRR
IPERTSHRVAGSPTPYPNYANAGHVEGQSALFMRDNGISEGLVFHNNPEGTCGFCVNMTETLLP
ENAKMTVVPPEGAIPVKRGATGETKVFTGNSNSPKSPTKGGCSGGSTNLSDIIEKETGKOLVIQ
ESILMLPEEVEEVIGNKPESDILVHTAYDESTDENVMLLTSDAPEYKPWALVIQDSNGENKIKM
L

General DdCBE Architecture and mitoTALE Sequences

### Legend:

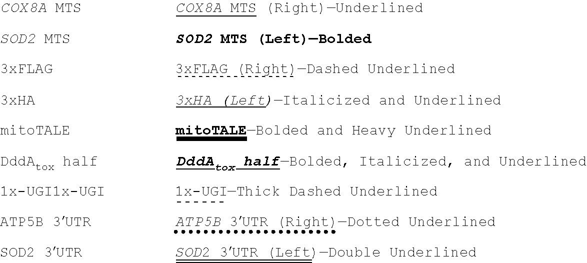

All right-side halves of DdCBEs have the general architecture of (from N- to C-terminus): COX8 Å MTS-3×FLAG-mitoTALE-2aa linker-DddAₜₒₓ half-4aa linker-1×-UGI-ATP5B 3′UTR

All left-side halves of DdCBEs have the general architecture of (from N- to C-terminus):

SOD2 MTS-3xHA-mitoTALE-2aa linker-DddAₜₒₓ half-4aa linker-1x-UGI- SOD2 3'UTR
(A) SOD2 MTS
(SEQ ID NO: 298)
MLSRAVCGTSRQLAPVLGYLGSROKHSLPD
(B) COX8A MTS
(SEQ ID NO: 299)
MSVLTPLLLRGLTGSARRLPVPRAKIHSL
(C) SOD2 3'UTR
(SEQ ID NO: 300)
ACCACGATCGTTATGCTGATCATACCCTAATGATCCCAGCAAGATAATGTCCTGTCTTCTAAGA
TGTGCATCAAGCCTGGTACATACTGAAAACCCTATAAGGTCCTGGATAATTTTTGTTTGATTAT
TCATTGAAGAAACATTTATTTTCCAATTGTGTGAAGTTTTTGACTGTTAATAAAAGAATCTGTC
AACCATCAAAAAAAAAAAAAAA
(D) ATP5B 3'UTR
(SEQ ID NO: 301)
ACCACGATCGTTATGCTGATCATACCCTAATGATCCCAGCAAGATAATGTCCTGTCTTCTAAGA
TGTGCATCAAGCCTGGTACATACTGAAAACCCTATAAGGTCCTGGATAATTTTTGTTTGATTAT
TCATTGAAGAAACATTTATTTTCCAATTGTGTGAAGTTTTTGACTGTTAATAAAAGAATCTGTC
AACCATCAAAAAAAAAAAAAAA
(E) ND6-DdCBE: Left mitoTALE-G1397-DddAtox-N-1x-UGI
(SEQ ID No: 302)
ATGGCCCTGTCCCGTGCGGTTTGTGGCACCTCCCGTCAACTGGCTCCGGTTCTGGGTTATCTGG
GTTCCCGTCAAAAACACTCCCTGCCGGACTACCCGTATGATGTTCCGGATTACGCTGGCTACCC
ATACGACGTCCCAGACTACGCTGGCTACCCATACGACGTCCCAGACTACGCTATGGACATCGCG
GATCTGCGTACCCTGGGTTACAGCCAGCAGCAGCAGGAGAAGATCAAGCCGAAGGTGCGCAGCA
CCGTGGCTCAGCACCACGAAGCCCTGGTGGGCCACGGTTTCACCCACGCTCACATTGTGGCCCT
GAGCCAGCACCCAGCCGCGCTGGGCACCGTGGCCGTGAAATATCAGGATATGATTGCTGCCCTG
CCAGAGGCCACCCATGAAGCTATTGTGGGCGTGGGCAAGCAGTGGAGCGGTGCTCGTGCGCTGG
AGGCGCTGCTGACCGTGGCTGGTGAACTGCGTGGTCCGCCGCTGCAGCTGGACACCGGTCAGCT
GCTGAAAATCGCGAAACGTGGCGGTGTGACCGCGGTGGAAGCCGTGCATGCTTGGCGTAATGCT
CTGACCGGTGCGCCGCTGAACCTGACCCCGCAGCAGGTGGTGGCTATTGCCAGCAACAACGGCG
GTAAACAGGCCCTGGAGACCGTGCAGCGCCTGCTGCCGGTGCTGTGCCAGGCCCATGGTCTGAC
CCCGGAGCAGGTGGTGGCGATCGCTAGCAACATCGGCGGCAAGCAGGCCCTGGAAACCGTGCAG
GCGCTGTTACCGGTGCTGTGCCAGGCTCATGGCCTGACCCCGGAACAAGTTGTGGCTATTGCCA
GCCATGATGGCGGTAAACAGGCTCTGGAAACCGTGCAGCGTCTGTTGCCGGTGCTGTGCCAAGC
CCATGGCCTGACCCCGGAGCAAGTTGTGGCTATTGCGAGCCATGATGGCGGCAAGCAGGCGCTG
GAAACCGTTCAGCGCCTGTTACCGGTGCTGTGCCAAGCTCATGGTCTGACCCCGGAACAGGTGG
TGGCCATTGCTTCCCATGATGGCGGTAAACAGGCCCTGGAAACCGTTCAGCGTCTGCTTCCGGT
GCTGTGCCAGGCCCATGGGCTGACCCCGGAACAAGTGGTTGCTATTGCCAGCCACGATGGCGGC
AAGCAGGCTCTGGAGACCGTTCAGCGCCTGCTTCCGGTGCTGTGCCAGGCCCATGGCTTAACCC
CGGAACAAGTTGTTGCTATTGCTAGTCATGATGGCGGTAAACAGGCGCTGGAGACCGTTCAGCG
TCTGTTACCGGTGCTGTGCCAGGCGCATGGCTTAACCCCGGAGCAGGTTGTTGCCATTGCCTCC
AATATCGGCGGCAAGCAGGCTCTGGAAACCGTTCAGGCCCTGTTGCCGGTGCTGTGCCAGGCCC
ATGGACTGACCCCGCAGCAAGTTGTTGCCATTGCCAGCAATGGCGGTGGCAAACAGGCGCTGGA
AACTGTTCAGCGCCTGCTCCCGGTGCTGTGCCAAGCGCATGGTCTGACCCCGCAGCAAGTGGTT
GCTATTGCTAGCAATGGTGGCGGTCGTCCGGCGCTGGAAAGCATTGTGGCTCAGCTGAGCCGTC
CAGACCCGGCCCTGGCGGCTCTGACCAACGATCACCTGGTGGCGCTGGCTTGCCTGGGCGGTCG
TCCGGCCCTGGATGCGGTGAAGAAAGGCCTGGGTGGATCCGGCAGCTACGCCCTGGGTCCGTAT
CAGATTAGCGCCCCGCAGCTGCCAGCTTACAATGGTCAGACCGTGGGTACCTTCTACTATGTGA
ACGACGCGGGCGGTCTGGAGAGCAAGGTGTTTAGCAGCGGCGGTCCAACCCCGTACCCAAACTA
TGCCAATGCCGGTCATGTGGAGGGTCAGAGCGCCCTGTTCATGCGTGATAACGGCATCAGCGAG
GGTCTGGTGTTCCACAACAACCCGGAAGGCACCTGCGGTTTTTGCGTGAACATGACCGAGACCC

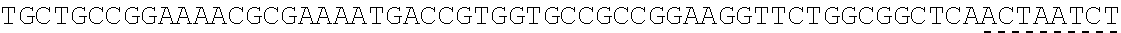

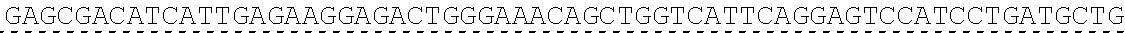

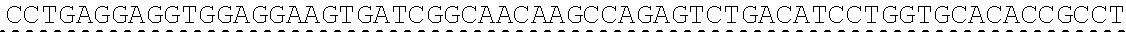

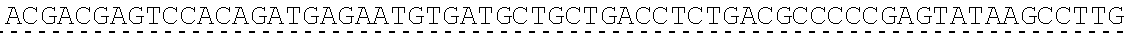

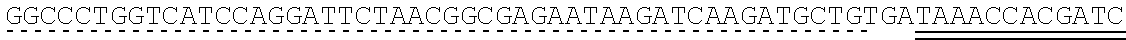

GTTATGCTGATCATACCCTAATGATCCCAGCAAGATAATGTCCTGTCTTCTAAGATGTGCATCA
AGCCTGGTACATACTGAAAACCCTATAAGGTCCTGGATAATTTTTGTTTGATTATTCATTGAAG
AAACATTTATTTTCCAATTGTGTGAAGTTTTTGACTGTTAATAAAAGAATCTGTCAACCATCAA
A
(F) ND6-DdCBE: Right mitoTALE-G1397-DddAtox-N-1x-UGI
(SEQ ID NO: 303)
TCCGTTCTGACCCCGCTGCTGCTGCGTGGCCTGACCGGCTCCGCTCGTCGTCTGCCAGTTCCGC

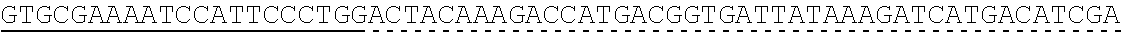

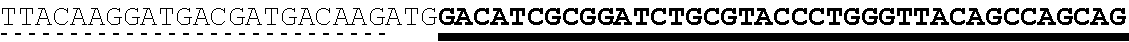

CAGCAGGAGAAGATCAAGCCAAAGGTGCGCAGCACCGTGGCCCAGCACCATGAAGCTCTGGTGG
GTCACGGCTTCACCCACGCGCACATCGTGGCTCTGAGCCAGCACCCAGCCGCGCTGGGTACCGT
GGCCGTGAAATATCAGGACATGATTGCTGCCCTGCCAGAGGCTACCCATGAAGCGATTGTGGGT
GTGGGCAAGAGAGGAGCCGGTGCTCGTGCGCTGGAGGCCCTGCTGACCGTGGCCGGTGAACTGC
GTGGCCCGCCGCTGCAGCTGGATACCGGCCAGCTGCTGAAAATCGCGAAACGTGGCGGTGTGAC
CGCTGTGGAAGCTGTGCATGCCTGGCGTAATGCTCTGACCGGTGCCCCGCTGAACCTGACCCCG
CAGCAGGTGGTGGCTATTGCCAGCAACAACGGCGGTAAACAGGCTCTGGAAACCGTGCAGCGCC
TGCTGCCGGTGCTGTGCCAGGCTCATGGTCTGACCCCGGAGCAGGTGGTGGCGATCGCTAGCAA
CATCGGCGGCAAGCAGGCTCTGGAGACCGTTCAGGCCCTGTTACCGGTGCTGTGCCAAGCCCAT
GGTCTGACCCCGCAGCAAGTTGTGGCTATTGCCAGCAATGGCGGTGGCAAACAGGCGCTGGAGA
CCGTGCAGCGTCTGTTGCCGGTGCTGTGCCAAGCCCATGGGCTGACCCCGCAGCAAGTGGTTGC
CATCGCCAGCAACAACGGTGGCAAGCAGGCCCTGGAGACCGTTCAGCGCCTGTTACCGGTGCTG
TGCCAGGCCCATGGCTTAACCCCGCAGCAAGTIGTGGCCATCGCTAGCAACAACGGTGGCAAAC
AGGCTCTGGAGACTGTTCAGCGTCTGCTTCCGGTGCTGTGCCAAGCGCATGGCCTGACCCCGGA
ACAAGTTGTTGCTATTGCCAGCCATGATGGTGGCAAGCAGGCGCTGGAAACCGTTCAGCGCCTG
CTTCCGGTGCTGTGCCAGGCGCATGGATTAACCCCGCAGCAAGTGGTGGCCATCGCCAGCAATG
GTGGCGGTAAACAGGCCCTGGAAACCGTTCAGCGTCTGTTACCGGTGCTGTGCCAGGCCCATGG
ATTAACCCCGGAACAAGTTGTGGCTATTGCGTCCAATATCGGCGGCAAGCAGGCGCTGGAAACT
GTGCAGGCTCTGCTCCCGGTGCTGTGCCAGGCCCATGGGTTAACCCCGCAGCAGGTTGTTGCCA
TTGCGAGCAACGGCGGTGGCAAACAGGCTCTGGAGACGGTTCAGCGCCTGCTCCCGGTGCTGTG
CCAGGCCCATGGTTTAACCCCGCAGCAGGTGGTTGCTATTGCTAGCAATGGCGGCGGCAAGCAG
GCGCTGGAAACGGTGCAGCGTCTGCTACCGGTGCTGTGCCAGGCACATGGCCTTACCCCGCAGC
AAGTTGTGGCCATTGCTAGCAATGGCGGTGGCCGTCCGGCCCTGGAAAGCATTGTGGCGCAGCT
GAGCCGTCCAGACCCGGCCCTGGCGGCTCTGACCAATGATCACCTGGTGGCCCTGGCCTGCCTG
GGTGGCCGTCCGGCTCTGGATGCCGTGAAGAAAGGTCTGGGCGGATCCGCCATTCCAGTGAAGC
GCGGCGCTACCGGTGAAACCAAAGTGTTTACCGGTAACAGCAACAGCCCGAAGAGCCCGACCAA

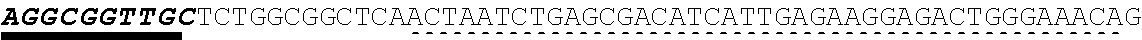

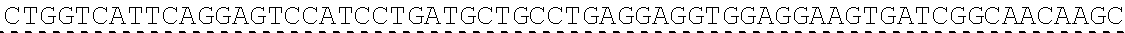

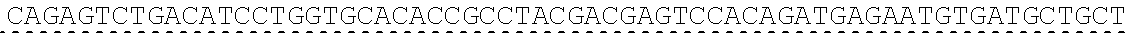

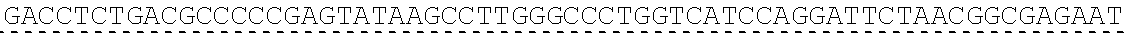

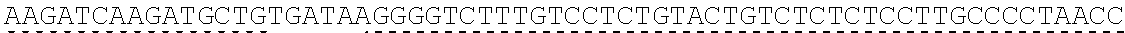

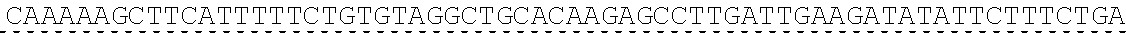

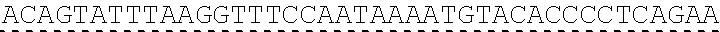

(G) ND1-DdCBE Right mitoTALE repeat
(SEQ ID NO: 304)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACAT
CGTGGCTCTGAGCCAGCACCCAGCCGCGCTGGGTACCGTGGCCGTGAAATATCAGGACATGATT
GCTGCCCTGCCAGAGGCTACCCATGAAGCGATTGTGGGTGTGGGCAAGCAGTGGAGCGGTGCTC
GTGCGCTGGAGGCCCTGCTGACCGTGGCCGGTGAACTGCGTGGCCCGCCGCTGCAGCTGGATAC
CGGCCAGCTGCTGAAAATCGCGAAACGTGGCGGTGTGACCGCTGTGGAAGCTGTGCATGCCTGG
CGTAATGCTCTGACCGGTGCCCCGCTGAACCTGACCCCGGAACAGGTGGTTGCCATCGCATCCA
ATAATGGTGGTAAACAAGCTCTGGAGACCGTTCAAGCCCTGCTGCCAGTGCTGTGCCAGGCTCA
TGGTCTGACCCCGCAGCAAGTIGTGGCTATTGCCAGCAACATCGGCGGCAAGCAGGCCCTGGAG
ACCGTGCAGCGTCTGCTGCCGGTGCTGTGCCAGGCCCATGGCCTGACCCCGCAGCAAGTGGTTG
CTATCGCCAGCAACAACGGCGGTAAACAGGCTCTGGAAACCGTGCAGCGCCTGTTACCGGTGCT
GTGCCAAGCCCATGGTCTGACCCCGGAGCAGGTGGTGGCGATTGCTAGCAACGGCGGTGGCAAG
CAGGCTCTGGAGACCGTTCAGGCCCTGCTTCCGGTGCTGTGCCAAGCGCATGGCCTGACCCCGG
AACAAGTTGTTGCCATTGCCAGCAATGGTGGCGGTAAACAGGCGCTGGAAACCGTGCAGGCTCT
GTTACCGGTGCTGTGCCAGGCCCATGGGCTGACCCCGGAGCAAGTGGTGGCTATTGCGAGCAAT
GGCGGTGGCAAGCAGGCCCTGGAAACCGTGCAGGCGCTGTTGCCGGTGCTGTGCCAAGCCCATG
GATTAACCCCGGAACAAGTGGTGGCGATCGCTAGCAACAACGGTGGCAAACAGGCGCTGGAGAC
CGTTCAGCGTCTGTTACCGGTGCTGTGCCAGGCGCATGGCTTAACCCCGGAACAGGTTGTTGCG
ATTGCCAGCAACATTGGTGGCAAGCAGGCTCTGGAAACCGTTCAGGCCCTGCTCCCGGTGCTGT
GCCAGGCCCATGGTTTAACCCCGGAACAGGTGGTGGCCATTGCCAGCAACGGTGGCGGTAAACA
GGCCCTGGAGACCGTTCAGCGCCTGCTACCGGTGCTGTGCCAGGCCCATGGACTGACCCCGGAG
CAAGTTGTTGCCATTGCTAGCAACAACGGCGGCAAGCAGGCGCTGGAGACCGTGCAGGCTCTGC
TTCCGGTGCTGTGCCAGGCCCATGGGTTAACCCCGGAGCAGGTTGTGGCCATCGCCAGCCACGA
CGGCGGTAAACAGGCCCTGGAAACCGTTCAGGCGCTGCTACCGGTGCTGTGCCAGGCACATGGC
TTAACCCCGGAGCAGGTGGTTGCCATCGCCTCCAATGGCGGTGGCAAGCAGGCTCTGGAAACGG
TGCAGGCCCTGCTGCCGGTGCTGTGCCAAGCCCATGGGTTGACCCCGGAACAAGTGGTGGCTAT
TGCTAGCCACGACGGTGGCAAACAGGCTCTGGAGACTGTTCAGCGTCTGCTTCCGGTGCTGTGC
CAGGCTCATGGCTTAACCCCGCAGCAAGTTGTTGCTATTGCCTCCAATATTGGTGGCAAGCAGG
CGCTGGAAACCGTTCAGCGCCTGCTGCCGGTGCTGTGCCAGGCTCATGGGCTTACCCCGGAACA
AGTTGTGGCCATTGCCTCCCATGATGGTGGCAAACAGGCGCTGGAAACTGTGCAGGCTCTGCTC
CCGGTGCTGTGCCAGGCTCATGGATTAACCCCGCAGCAAGTGGTGGCCATTGCTAGCCACGATG
GTGGCAAGCAGGCCCTGGAGACGGTTCAGCGTCTGCTCCCGGTGCTGTGCCAGGCCCATGGGCT
AACCCCGCAGCAGGTTGTTGCTATTGCCAGTCATGATGGTGGCAAACAGGCTCTGGAAACTGTG
CAGCGCCTGCTACCGGTGCTGTGCCAGGCTCACGGTCTGACCCCGCAGCAGGTGGTGGCAATCG
CAAGCAACGGTGGTGGTCGTCCGGCACTGGAAAGCATTGTGGCGCAGCTGAGCCGTCCAGACCC
GGCCCTGGCGGCTCTGACCAATGATCACCTGGTGGCCCTGGCCTGCCTGGGTGGCCGTCCGGCT
CTGGATGCCGTGAAGAAAGGTCTGGGC
Translated amino acid sequence :
(SEQ ID NO: 305)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPEQVVAIASNNGGKQALETVQALLPVLCQAHGLTPQQVVAIASNIGGKQALE
TVQRLLPVLCQAHGLTPQQVVAIASNNGGKQALETVORLLPVLCQAHGLTPEQVVAIASNGGGK
QALETVQALLPVLCQAHGLTPEQVVAIASNGGGKQALETVQALLPVLCQAHGLTPEQVVAIASN
GGGKQALETVQALLPVLCQAHGLTPEQVVAIASNNGGKQALETVQRLLPVLCQAHGLTPEQVVA
IASNIGGKQALETVQALLPVLCQAHGLTPEQVVAIASNGGGKQALETVORLLPVLCQAHGLTPE
QVVAIASNNGGKQALETVQALLPVLCQAHGLTPEQVVAIASHDGGKQALETVQALLPVLCQAHG
LTPEQVVAIASNGGGKQALETVQALLPVLCQAHGLTPEQVVAIASHDGGKQALETVORLLPVLC
QAHGLTPQQVVAIASNIGGKQALETVORLLPVLCQAHGLTPEQVVAIASHDGGKOALETVQALL
PVLCQAHGLTPQQVVAIASHDGGKQALETVQRLLPVLCQAHGLTPQQVVAIASHDGGKQALETV
QRLLPVLCQAHGLTPQQVVAIASNGGGRPALESIVAQLSRPDPALAALTNDHLVALACLGGRPA
LDAVKKGLG
(H) ND1-DdCBE Left mitoTALE repeat
(SEQ ID NO: 306)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACAT
TGTGGCCCTGAGCCAGCACCCAGCCGCGCTGGGCACCGTGGCCGTGAAATATCAGGATATGATT
GCTGCCCTGCCAGAGGCCACCCATGAAGCTATTGTGGGCGTGGGCAAGCAGTGGAGCGGTGCTC
GTGCGCTGGAGGCGCTGCTGACCGTGGCTGGTGAACTGCGTGGTCCGCCGCTGCAGCTGGACAC
CGGTCAGCTGCTGAAAATCGCGAAACGTGGCGGTGTGACCGCGGTGGAAGCCGTGCATGCTTGG
CGTAATGCTCTGACCGGTGCGCCGCTGAACCTGACCCCGGAACAAGTGGTTGCTATCGCATCCC
ATGACGGCGGTAAACAAGCCCTGGAGACCGTTCAAGCCCTGCTGCCAGTGCTGTGCCAGGCTCA
TGGTCTGACCCCGCAGCAGGTGGTGGCTATTGCCAGCAATGGCGGTGGCAAGCAGGCGCTGGAG
ACCGTGCAGCGTCTGCTGCCGGTGCTGTGCCAAGCCCATGGCCTGACCCCGCAGCAAGTIGTGG
CTATCGCCAGCAACATTGGTGGCAAACAGGCCCTGGAAACCGTGCAGCGCCTGTTACCGGTGCT
GTGCCAGGCCCATGGTCTGACCCCGGAGCAGGTGGTGGCGATCGCTAGCAACAACGGTGGCAAG
CAGGCTCTGGAAACCGTGCAGGCCCTGCTTCCGGTGCTGTGCCAGGCCCATGGGCTGACCCCGG
AACAAGTTGTGGCTATTGCCAGCCACGACGGTGGCAAACAGGCGCTGGAAACCGTGCAGGCTCT
GTTACCGGTGCTGTGCCAAGCGCATGGCCTGACCCCGGAACAGGTGGTGGCTATTGCTAGCCAC
GATGGTGGCAAGCAGGCCCTGGAGACCGTTCAGGCGCTGTTGCCGGTGCTGTGCCAGGCGCATG
GCTTAACCCCGGAACAAGTTGTTGCGATTGCTAGCAACGGTGGCGGTAAACAGGCTCTGGAGAC
CGTTCAGCGTCTGTTACCGGTGCTGTGCCAGGCACATGGCCTGACCCCGGAGCAAGTTGTTGCC
ATTGCCAGCAACATCGGCGGCAAGCAGGCTCTGGAGACCGTGCAGGCCCTGCTCCCGGTGCTGT
GCCAGGCCCATGGCTTAACCCCGGAGCAAGTTGTGGCCATTGCCAGCAACAACGGCGGTAAACA
GGCGCTGGAGACCGTTCAGCGCCTGCTACCGGTGCTGTGCCAGGCTCATGGCTTGACCCCGGAA
CAGGTTGTTGCGATTGCGAGCCATGATGGCGGCAAGCAGGCGCTGGAAACCGTTCAGGCTCTGC
TTCCGGTGCTGTGCCAGGCCCATGGATTAACCCCGGAGCAGGTTGTTGCTATTGCCAGCCATGA
TGGCGGTAAACAGGCCCTGGAGACCGTGCAGGCGCTGCTACCGGTGCTGTGCCAGGCTCATGGG
CTGACCCCGGAGCAAGTGGTTGCTATCGCGAGCAACAATGGCGGCAAGCAGGCTCTGGAAACGG
TGCAGGCCCTGCTGCCGGTGCTGTGCCAGGCCCATGGGTTAACCCCGGAACAAGTGGTGGCCAT
CGCTAGCAACGGCGGTGGCAAACAGGCCCTGGAGACTGTTCAGCGTCTGCTTCCGGTGCTGTGC
CAGGCCCATGGGCTAACCCCGCAGCAAGTGGTTGCCATTGCCAGCAATGGCGGCGGCAAGCAGG
CTCTGGAAACTGTGCAGCGCCTGCTGCCGGTGCTGTGCCAGGCTCACGGTCTGACCCCGCAACA
GGTGGTGGCAATCGCAAGCAATGGTGGTGGTCGTCCGGCACTGGAGAGCATTGTGGCTCAGCTG
AGCCGTCCAGACCCGGCCCTGGCGGCTCTGACCAACGATCACCTGGTGGCGCTGGCTTGCCTGG
GCGGTCGTCCGGCCCTGGATGCGGTGAAGAAAGGCCTGGGT
Translated amino acid sequence :
(SEQ ID NO: 307)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPEQVVAIASHDGGKQALETVQALLPVLCQAHGLTPQQVVAIASNGGGKQALE
TVQRLLPVLCQAHGLTPQQVVAIASNIGGKQALETVORLLPVLCQAHGLTPEQVVAIASNNGGK
QALETVQALLPVLCQAHGLTPEQVVAIASHDGGKQALETVQALLPVLCQAHGLTPEQVVAIASH
DGGKQALETVQALLPVLCQAHGLTPEQVVAIASNGGGKQALETVORLLPVLCQAHGLTPEQVVA
IASNIGGKQALETVQALLPVLCQAHGLTPEQVVAIASNNGGKQALETVORLLPVLCQAHGLTPE
QVVAIASHDGGKQALETVQALLPVLCQAHGLTPEQVVAIASHDGGKQALETVQALLPVLCQAHG
LTPEQVVAIASNNGGKQALETVQALLPVLCQAHGLTPEQVVAIASNGGGKQALETVORLLPVLC
QAHGLTPQQVVAIASNGGGKQALETVORLLPVLCQAHGLTPQQVVAIASNGGGRPALESIVAQL
SRPDPALAALTNDHLVALACLGGRPALDAVKKGLG
(I) ND2-DdCBE Right mitoTALE repeat
(SEQ ID NO: 308)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACAT
CGTGGCTCTGAGCCAGCACCCAGCCGCGCTGGGTACCGTGGCCGTGAAATATCAGGACATGATT
GCTGCCCTGCCAGAGGCTACCCATGAAGCGATTGTGGGTGTGGGCAAGCAGTGGAGCGGTGCTC
GTGCGCTGGAGGCCCTGCTGACCGTGGCCGGTGAACTGCGTGGCCCGCCGCTGCAGCTGGATAC
CGGCCAGCTGCTGAAAATCGCGAAACGTGGCGGTGTGACCGCTGTGGAAGCTGTGCATGCCTGG
CGTAATGCTCTGACCGGTGCCCCGCTGAACCTGACCCCGGAACAAGTGGTTGCCATCGCATCCA
ATATCGGTGGTAAACAAGCCCTGGAGACCGTTCAAGCCCTGCTGCCAGTGCTGTGCCAGGCTCA
TGGTCTGACCCCGCAGCAGGTGGTGGCCATTGCGAGCAACAATGGCGGCAAGCAGGCGCTGGAG
ACCGTGCAGCGTCTGCTGCCGGTGCTGTGCCAAGCCCATGGCCTGACCCCGCAGCAAGTGGTTG
CTATCGCCAGCAACATTGGCGGTAAACAGGCCCTGGAAACCGTGCAGCGCCTGTTACCGGTGCT
GTGCCAGGCCCATGGTCTGACCCCGGAGCAGGTGGTGGCGATCGCTAGCAACATCGGCGGCAAG
CAGGCTCTGGAAACCGTGCAGGCCCTGCTTCCGGTGCTGTGCCAGGCCCATGGGCTGACCCCGG
AACAAGTTGTGGCTATTGCCAGCCATGATGGCGGTAAACAGGCGCTGGAAACCGTGCAGGCTCT
GTTACCGGTGCTGTGCCAAGCGCATGGCCTGACCCCGGAACAGGTGGTGGCTATTGCGAGCAAT
GGCGGTGGCAAGCAGGCCCTGGAGACCGTTCAGGCGCTGTTGCCGGTGCTGTGCCAGGCGCATG
GCTTAACCCCGGAACAAGTTGTTGCGATCGCTAGCAACAACGGTGGCAAACAGGCTCTGGAGAC
CGTTCAGCGTCTGTTACCGGTGCTGTGCCAGGCACATGGCCTGACCCCGGAGCAAGTTGTTGCC
ATTGCCAGCCACGATGGTGGCAAGCAGGCTCTGGAGACCGTGCAGGCCCTGCTCCCGGTGCTGT
GCCAGGCCCATGGCTTAACCCCGGAGCAAGTIGTGGCTATCGCCAGCAACGGTGGCGGTAAACA
GGCGCTGGAGACCGTTCAGCGCCTGCTACCGGTGCTGTGCCAGGCTCATGGCTTGACCCCGGAA
CAGGTTGTTGCCATTGCGTCCAATATCGGCGGCAAGCAGGCGCTGGAAACCGTTCAGGCTCTGC
TTCCGGTGCTGTGCCAGGCCCATGGATTAACCCCGGAGCAGGTTGTGGCGATTGCGAGCAACGG
CGGTGGCAAACAGGCCCTGGAGACCGTGCAGGCGCTGCTACCGGTGCTGTGCCAGGCTCATGGG
CTGACCCCGGAGCAAGTGGTTGCTATTGCTAGCAATGGCGGCGGCAAGCAGGCTCTGGAAACGG
TGCAGGCCCTGCTGCCGGTGCTGTGCCAGGCCCATGGGTTAACCCCGGAACAAGTGGTGGCCAT
CGCTTCCAATATTGGCGGTAAACAGGCCCTGGAGACTGTTCAGCGTCTGCTTCCGGTGCTGTGC
CAGGCCCATGGGCTAACCCCGCAGCAAGTTGTTGCTATTGCCTCCAATGGCGGTGGCAAGCAGG
CTCTGGAAACTGTGCAGCGCCTGCTGCCGGTGCTGTGCCAGGCTCACGGCCTGACCCCGCAGCA
AGTTGTGGCAATCGCAAGCAATGGTGGTGGTCGTCCGGCTCTGGAGAGCATTGTGGCGCAGCTG
AGCCGTCCAGACCCGGCCCTGGCGGCTCTGACCAATGATCACCTGGTGGCCCTGGCCTGCCTGG
GTGGCCGTCCGGCTCTGGATGCCGTGAAGAAAGGTCTGGGC
Translated amino acid sequence:
(SEQ ID NO: 309)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPEQVVAIASNIGGKQALETVQALLPVLCQAHGLTPQQVVAIASNNGGKQALE
TVQRLLPVLCQAHGLTPQQVVAIASNIGGKQALETVORLLPVLCQAHGLTPEQVVAIASNIGGK
QALETVQALLPVLCQAHGLTPEQVVAIASHDGGKQALETVQALLPVLCQAHGLTPEQVVAIASN
GGGKQALETVQALLPVLCQAHGLTPEQVVAIASNNGGKQALETVORLLPVLCQAHGLTPEQVVA
IASHDGGKQALETVQALLPVLCQAHGLTPEQVVAIASNGGGKQALETVORLLPVLCQAHGLTPE
QVVAIASNIGGKQALETVQALLPVLCQAHGLTPEQVVAIASNGGGKQALETVQALLPVLCQAHG
LTPEQVVAIASNGGGKQALETVQALLPVLCQAHGLTPEQVVAIASNIGGKQALETVORLLPVLC
QAHGLTPQQVVAIASNGGGKQALETVQRLLPVLCQAHGLTPQQVVAIASNGGGRPALESIVAQL
SRPDPALAALTNDHLVALACLGGRPALDAVKKGLG
(J) ND2-DdCBE Left mitoTALE repeat
(SEQ ID NO: 310)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACAT
CGTGGCTCTGAGCCAGCACCCAGCCGCGCTGGGTACCGTGGCCGTGAAATATCAGGACATGATT
GCTGCCCTGCCAGAGGCTACCCATGAAGCGATTGTGGGTGTGGGCAAGCAGTGGAGCGGTGCTC
GTGCGCTGGAGGCCCTGCTGACCGTGGCCGGTGAACTGCGTGGCCCGCCGCTGCAGCTGGATAC
CGGCCAGCTGCTGAAAATCGCGAAACGTGGCGGTGTGACCGCTGTGGAAGCTGTGCATGCCTGG
CGTAATGCTCTGACCGGTGCCCCGCTGAACCTGACCCCGGAACAAGTGGTGGCTATCGCGTCCC
ATGATGGTGGTAAACAGGCTCTGGAGACCGTGCAAGCTCTGCTGCCAGTGCTGTGCCAGGCCCA
TGGTCTGACCCCGCAGCAGGTGGTGGCTATTGCCAGCAATGGCGGTGGCAAGCAGGCGCTGGAG
ACCGTGCAGCGTCTGCTGCCGGTGCTGTGCCAGGCTCATGGCCTGACCCCGCAGCAAGTIGTGG
CTATTGCCAGCAACGGTGGCGGTAAACAGGCCCTGGAGACCGTGCAGCGCCTGTTACCGGTGCT
GTGCCAAGCCCATGGCCTGACCCCGGAGCAGGTGGTGGCGATCGCTAGCAACATCGGCGGCAAG
CAGGCTCTGGAAACCGTGCAGGCCCTGCTTCCGGTGCTGTGCCAGGCCCATGGCTTAACCCCGG
AACAGGTTGTTGCTATTGCCAGCAACAACGGCGGTAAACAGGCGCTGGAAACCGTGCAGGCTCT
GTTACCGGTGCTGTGCCAGGCCCATGGGCTGACCCCGGAACAAGTTGTGGCTATTGCGAGCCAT
GATGGCGGCAAGCAGGCCCTGGAAACCGTGCAGGCGCTGTTGCCGGTGCTGTGCCAAGCCCATG
GATTAACCCCGGAACAAGTTGTTGCGATCGCTAGCAACATTGGCGGTAAACAGGCTCTGGAAAC
CGTTCAGCGTCTGTTACCGGTGCTGTGCCAGGCGCATGGTCTGACCCCGGAACAGGTTGTGGCC
ATTGCCTCCAATGGCGGTGGCAAGCAGGCTCTGGAGACCGTTCAGGCCCTGCTCCCGGTGCTGT
GCCAAGCGCATGGCCTGACCCCGGAACAGGTGGTGGCTATCGCCAGCAACATTGGTGGCAAACA
GGCGCTGGAGACCGTTCAGCGCCTGCTACCGGTGCTGTGCCAGGCACATGGTCTGACCCCGGAG
CAAGTTGTGGCCATTGCTAGCCACGATGGTGGCAAGCAGGCGCTGGAAACCGTTCAGGCTCTGC
TTCCGGTGCTGTGCCAGGCCCATGGTTTAACCCCGGAACAAGTGGTTGCCATTGCGTCCAATGG
TGGCGGTAAACAGGCCCTGGAAACCGTTCAGGCGCTGCTACCGGTGCTGTGCCAGGCTCATGGG
CTGACCCCGGAGCAAGTGGTTGCTATTGCTTCCCATGATGGCGGCAAGCAGGCTCTGGAAACGG
TGCAGGCCCTGCTGCCGGTGCTGTGCCAAGCCCATGGGTTAACCCCGGAACAGGTGGTTGCGAT
TGCTAGCCACGACGGCGGTAAACAGGCCCTGGAAACGGTTCAGCGTCTGCTTCCGGTGCTGTGC
CAGGCCCATGGACTTACCCCGCAGCAGGTTGTGGCGATTGCCTCCAATGGCGGTGGCAAGCAGG
CTCTGGAAACTGTGCAGCGCCTGCTGCCGGTGCTGTGCCAGGCTCATGGTTTAACCCCGGAGCA
GGTTGTTGCCATCGCCAGCCACGACGGTGGCAAACAGGCGCTGGAAACTGTGCAGGCTCTGCTC
CCGGTGCTGTGCCAGGCTCATGGACTTACCCCGGAGCAGGTGGTTGCCATTGCTAGCAACATTG
GTGGCAAGCAGGCCCTGGAGACTGTTCAGGCGCTGTTACCGGTGCTGTGCCAGGCCCATGGGTT
AACCCCGGAGCAAGTTGTTGCCATTGCCTCCAATATTGGTGGCAAACAGGCTCTGGAGACTGTT
CAGGCCCTGCTGCCGGTGCTGTGCCAGGCTCACGGTCTGACCCCGCAGCAAGTGGTGGCAATCG
CAAGCAATGGTGGTGGTCGTCCGGCTCTGGAGAGCATTGTGGCGCAGCTGAGCCGTCCAGACCC
GGCCCTGGCGGCTCTGACCAATGATCACCTGGTGGCCCTGGCCTGCCTGGGTGGCCGTCCGGCT
CTGGATGCCGTGAAGAAAGGTCTGGGC
Translated amino acid sequence:
(SEQ ID NO: 311)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPEQVVAIASNIGGKQALETVQALLPVLCQAHGLTPQQVVAIASNNGGKQALE
TVQRLLPVLCQAHGLTPQQVVAIASNIGGKQALETVQRLLPVLCQAHGLTPEQVVAIASNIGGK
QALETVQALLPVLCQAHGLTPEQVVAIASHDGGKQALETVQALLPVLCQAHGLTPEQVVAIASN
GGGKQALETVQALLPVLCQAHGLTPEQVVAIASNNGGKQALETVORLLPVLCQAHGLTPEQVVA
IASHDGGKQALETVQALLPVLCQAHGLTPEQVVAIASNGGGKQALETVORLLPVLCQAHGLTPE
QVVAIASNIGGKQALETVQALLPVLCQAHGLTPEQVVAIASNGGGKQALETVQALLPVLCQAHG
LTPEQVVAIASNGGGKQALETVQALLPVLCQAHGLTPEQVVAIASNIGGKQALETVQRLLPVLC
QAHGLTPQQVVAIASNGGGKQALETVORLLPVLCQAHGLTPQQVVAIASNGGGRPALESIVAQL
SRPDPALAALTNDHLVALACLGGRPALDAVKKGLG
(K) ND4-DdCBE Right mitoTALE repeat
(SEQ ID NO: 312)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACAT
CGTGGCTCTGAGCCAGCACCCAGCCGCGCTGGGTACCGTGGCCGTGAAATATCAGGACATGATT
GCTGCCCTGCCAGAGGCTACCCATGAAGCGATTGTGGGTGTGGGCAAGCAGTGGAGCGGTGCTC
GTGCGCTGGAGGCCCTGCTGACCGTGGCCGGTGAACTGCGTGGCCCGCCGCTGCAGCTGGATAC
CGGCCAGCTGCTGAAAATCGCGAAACGTGGCGGTGTGACCGCTGTGGAAGCTGTGCATGCCTGG
CGTAATGCTCTGACCGGTGCCCCGCTGAACCTGACCCCGGAACAAGTGGTTGCGATTGCGTCCC
ATGATGGTGGTAAACAAGCCCTGGAGACCGTTCAAGCTCTGCTGCCAGTGCTGTGCCAGGCTCA
TGGTCTGACCCCGCAGCAGGTGGTGGCTATTGCCAGCCATGATGGCGGCAAGCAGGCTCTGGAG
ACCGTGCAGCGTCTGCTGCCGGTGCTGTGCCAAGCCCATGGCCTGACCCCGCAGCAAGTIGTGG
CTATTGCCAGCAACGGCGGTGGCAAACAGGCGCTGGAAACCGTGCAGCGCCTGTTACCGGTGCT
GTGCCAAGCGCATGGTCTGACCCCGGAGCAGGTGGTGGCCATCGCTAGCAACAACGGTGGCAAG
CAGGCCCTGGAAACCGTGCAGGCGCTGTTGCCGGTGCTGTGCCAGGCCCATGGCTTAACCCCGG
AACAAGTGGTGGCCATTGCGAGCAATGGTGGCGGTAAACAGGCTCTGGAAACCGTGCAGGCCCT
GCTTCCGGTGCTGTGCCAGGCCCATGGGCTGACCCCGGAACAAGTIGTGGCTATCGCCAGCAAC
ATCGGCGGCAAGCAGGCGCTGGAGACCGTTCAGGCTCTGTTACCGGTGCTGTGCCAGGCGCATG
GCCTGACCCCGGAACAGGTGGTGGCGATCGCTAGCAACATTGGCGGTAAACAGGCCCTGGAGAC
CGTTCAGCGCCTGCTCCCGGTGCTGTGCCAGGCCCATGGTCTGACCCCGGAACAGGTTGTTGCT
ATTGCCAGCAACAACGGCGGCAAGCAGGCCCTGGAGACCGTGCAGGCGCTGCTACCGGTGCTGT
GCCAGGCCCATGGACTGACCCCGGAGCAGGTTGTGGCCATCGCGTCCAATGGCGGTGGCAAACA
GGCTCTGGAGACCGTTCAGCGTCTGTTACCGGTGCTGTGCCAGGCACATGGCCTGACCCCGGAG
CAAGTTGTTGCCATCGCTAGCAACATTGGTGGCAAGCAGGCGCTGGAAACCGTTCAGCGCCTGC
TACCGGTGCTGTGCCAGGCTCATGGCTTAACCCCGGAGCAGGTTGTCGCCATTGCCAGCAACAA
TGGTGGCAAACAGGCTCTGGAAACTGTGCAGGCCCTGCTACCGGTGCTGTGCCAGGCCCATGGG
TTAACCCCGGAACAGGTTGTGGCCATTGCCTCCAATAACGGTGGCAAGCAGGCGCTGGAAACGG
TGCAGGCTCTGCTTCCGGTGCTGTGCCAGGCTCATGGGCTGACCCCGGAGCAAGTGGTTGCTAT
TGCGTCCAACATTGGTGGCAAACAGGCCCTGGAAACCGTTCAGGCGCTGCTCCCGGTGCTGTGC
CAGGCCCATGGGCTAACCCCGGAACAGGTGGTTGCCATTGCCTCCAACAATGGTGGCAAGCAGG
CCCTGGAAACGGTTCAGCGTCTGCTTCCGGTGCTGTGCCAGGCCCATGGGCTTACCCCGCAGCA
AGTTGTTGCTATCGCCAGCAATATTGGTGGCAAACAGGCTCTGGAAACGGTGCAGCGCCTGCTA
CCGGTGCTGTGCCAGGCTCATGGTTTAACCCCGCAGCAGGTGGTTGCGATTGCCTCCAACAACG
GTGGCAAGCAGGCGCTGGAAACTGTTCAGCGTCTGCTCCCGGTGCTGTGCCAGGCTCACGGCCT
GACCCCGCAGCAAGTGGTGGCTATCGCCTCCAACGGTGGTGGTCGCCCGGCTCTGGAAAGCATT
GTGGCGCAGCTGAGCCGTCCAGACCCGGCCCTGGCGGCTCTGACCAATGATCACCTGGTGGCCC
TGGCCTGCCTGGGTGGCCGTCCGGCTCTGGATGCCGTGAAGAAAGGTCTGGGC
Translated amino acid sequence:
(SEQ ID NO: 313)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPEQVVAIASHDGGKQALETVQALLPVLCQAHGLTPQQVVAIASHDGGKQALE
TVQRLLPVLCQAHGLTPQQVVAIASNGGGKQALETVORLLPVLCQAHGLTPEQVVAIASNNGGK
QALETVQALLPVLCQAHGLTPEQVVAIASNGGGKQALETVQALLPVLCQAHGLTPEQVVAIASN
IGGKQALETVQALLPVLCQAHGLTPEQVVAIASNIGGKQALETVORLLPVLCQAHGLTPEQVVA
IASNNGGKQALETVQALLPVLCQAHGLTPEQVVAIASNGGGKQALETVORLLPVLCQAHGLTPE
QVVAIASNIGGKQALETVORLLPVLCQAHGLTPEQVVAIASNNGGKQALETVQALLPVLCQAHG
LTPEQVVAIASNNGGKQALETVQALLPVLCQAHGLTPEQVVAIASNIGGKQALETVQALLPVLC
QAHGLTPEQVVAIASNNGGKQALETVORLLPVLCQAHGLTPQQVVAIASNIGGKQALETVORLL
PVLCQAHGLTPQQVVAIASNNGGKQALETVORLLPVLCQAHGLTPQQVVAIASNGGGRPALESI
VAQLSRPDPALAALTNDHLVALACLGGRPALDAVKKGLG
(L) ND4-DdCBE Left mitoTALE repeat
(SEQ ID NO: 314)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACAT
CGTGGCTCTGAGCCAGCACCCAGCCGCGCTGGGTACCGTGGCCGTGAAATATCAGGACATGATT
GCTGCCCTGCCAGAGGCTACCCATGAAGCGATTGTGGGTGTGGGCAAGCAGTGGAGCGGTGCTC
GTGCGCTGGAGGCCCTGCTGACCGTGGCCGGTGAACTGCGTGGCCCGCCGCTGCAGCTGGATAC
CGGCCAGCTGCTGAAAATCGCGAAACGTGGCGGTGTGACCGCTGTGGAAGCTGTGCATGCCTGG
CGTAATGCTCTGACCGGTGCCCCGCTGAACCTGACCCCGGAACAGGTGGTGGCAATCGCAAGCA
ATAATGGTGGTAAACAGGCTCTGGAAACCGTGCAAGCTCTGCTGCCAGTTCTGTGCCAGGCTCA
TGGTCTGACCCCGCAGCAGGTGGTGGCTATTGCCAGCCATGATGGCGGCAAGCAGGCCCTGGAG
ACCGTGCAGCGTCTGCTGCCGGTGCTGTGCCAGGCCCATGGCCTGACCCCGCAGCAAGTIGTGG
CTATTGCCAGCAACGGCGGTGGCAAACAGGCTCTGGAGACCGTGCAGCGCCTGTTACCGGTGCT
GTGCCAAGCCCATGGTCTGACCCCGGAGCAGGTGGTGGCGATCGCTAGCAACATTGGTGGCAAG
CAGGCCCTGGAAACCGTGCAGGCGCTGTTGCCGGTGCTGTGCCAAGCCCATGGGCTGACCCCGG
AACAAGTTGTTGCCATTGCCAGCAACAATGGTGGCAAACAGGCTCTGGAAACTGTGCAGGCCCT
GCTTCCGGTGCTGTGCCAGGCCCATGGATTAACCCCGGAACAAGTIGTGGCTATTGCGAGCAAT
GGCGGCGGCAAGCAGGCGCTGGAAACCGTGCAGGCTCTGTTACCGGTGCTGTGCCAGGCGCATG
GCCTGACCCCGGAGCAAGTGGTGGCCATCGCTAGCAACATTGGCGGTAAACAGGCGCTGGAGAC
CGTTCAGCGTCTGTTACCGGTGCTGTGCCAGGCACATGGCCTTACCCCGGAACAAGTIGTGGCC
ATTGCCAGCAACATCGGCGGCAAGCAGGCCCTGGAAACGGTGCAGGCGCTGCTCCCGGTGCTGT
GCCAGGCCCATGGGTTAACCCCGGAACAAGTGGTTGCTATTGCTAGCCATGATGGCGGTAAACA
GGCCCTGGAGACCGTTCAGCGCCTGCTACCGGTGCTGTGCCAGGCCCATGGTTTAACCCCGGAA
CAGGTTGTTGCGATTGCTAGCCACGATGGCGGCAAGCAGGCTCTGGAGACCGTTCAGGCCCTGC
TCCCGGTGCTGTGCCAGGCCCATGGGCTTACCCCGGAGCAAGTTGTTGCTATTGCCTCCAATAT
TGGCGGTAAACAGGCGCTGGAAACCGTTCAGGCTCTGCTTCCGGTGCTGTGCCAGGCTCATGGC
CTCACCCCGGAACAAGTIGTGGCGATTGCGTCCCATGATGGCGGCAAGCAGGCCCTGGAAACTG
TGCAGGCGCTGCTACCGGTGCTGTGCCAGGCCCATGGGCTAACCCCGGAACAGGTGGTTGCGAT
TGCTAGCAACAACGGCGGTAAACAGGCTCTGGAGACTGTTCAGCGTCTGCTTCCGGTGCTGTGC
CAGGCTCATGGGCTGACCCCGCAGCAAGTGGTTGCTATTGCCAGCAATGGCGGTGGCAAGCAGG
CGCTGGAGACTGTTCAGCGCCTGCTCCCGGTGCTGTGCCAGGCTCATGGTTTAACCCCGGAGCA
GGTTGTGGCGATCGCCAGCAATGGTGGCGGTAAACAGGCTCTGGAAACGGTGCAGGCCCTGCTC
CCGGTGCTGTGCCAGGCTCATGGACTGACCCCGGAGCAAGTTGTTGCCATTGCGTCCCACGACG
GCGGCAAGCAGGCGCTGGAGACGGTGCAGGCTCTGCTCCCGGTGCTGTGCCAGGCTCACGGTCT
GACCCCGCAACAGGTGGTGGCAATCGCAAGCAACGGTGGTGGTCGTCCGGCACTGGAGAGCATT
GTGGCGCAGCTGAGCCGTCCAGACCCGGCCCTGGCGGCTCTGACCAATGATCACCTGGTGGCCC
TGGCCTGCCTGGGTGGCCGTCCGGCTCTGGATGCCGTGAAGAAAGGTCTGGGC
Translated amino acid sequence:
(SEQ ID NO: 315)
DIADLRTLGYSQQQQEKIKPKVRSTVAQHHEALVGHGFTHAHIVALSQHPAALGTVAVKYQDMI
AALPEATHEAIVGVGKQWSGARALEALLTVAGELRGPPLQLDTGOLLKIAKRGGVTAVEAVHAW
RNALTGAPLNLTPEQVVAIASNNGGKQALETVQALLPVLCQAHGLTPQQVVAIASHDGGKQALE
TVQRLLPVLCQAHGLTPQQVVAIASNGGGKQALETVORLLPVLCQAHGLTPEQVVAIASNIGGK
QALETVQALLPVLCQAHGLTPEQVVAIASNNGGKQALETVQALLPVLCQAHGLTPEQVVAIASN
GGGKQALETVQALLPVLCQAHGLTPEQVVAIASNIGGKQALETVORLLPVLCQAHGLTPEQVVA
IASNIGGKQALETVQALLPVLCQAHGLTPEQVVAIASHDGGKQALETVORLLPVLCQAHGLTPE
QVVAIASHDGGKQALETVQALLPVLCQAHGLTPEQVVAIASNIGGKQALETVQALLPVLCQAHG
LTPEQVVAIASHDGGKQALETVQALLPVLCQAHGLTPEQVVAIASNNGGKQALETVQRLLPVLC
QAHGLTPQQVVAIASNGGGKQALETVQRLLPVLCQAHGLTPEQVVAIASNGGGKQALETVQALL
PVLCQAHGLTPEQVVAIASHDGGKQALETVQALLPVLCQAHGLTPQQVVAIASNGGGRPALESI
VAQLSRPDPALAALTNDHLVALACLGGRPALDAVKKGLG
(M) ND5.1-DdCBE Right mitoTALE repeat
(SEQ ID NO: 316)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACAT
CGTGGCTCTGAGCCAGCACCCAGCCGCGCTGGGTACCGTGGCCGTGAAATATCAGGACATGATT
GCTGCCCTGCCAGAGGCTACCCATGAAGCGATTGTGGGTGTGGGCAAGCAGTGGAGCGGTGCTC
GTGCGCTGGAGGCCCTGCTGACCGTGGCCGGTGAACTGCGTGGCCCGCCGCTGCAGCTGGATAC
CGGCCAGCTGCTGAAAATCGCGAAACGTGGCGGTGTGACCGCTGTGGAAGCTGTGCATGCCTGG
CGTAATGCTCTGACCGGTGCCCCGCTGAACCTGACCCCACAGCAGGTGGTGGCAATCGCAAGCC
ACGACGGAGGCAAGCAGGCCCTGGAGACCGTGCAGAGGCTGCTGCCCGTGCTGTGCCAGGCACA
CGGACTGACACCTGAACAGGTCGTGGCAATCGCATCCAACGGAGGCGGCAAGCAGGCCCTGGAA
ACCGTGCAGCGCCTGTTACCCGTGCTGTGCCAGGCCCACGGCCTGACACCCCAGCAGGTGGTGG
CCATCGCCTCTAATGGAGGGGGCAAGCAGGCCCTGGAGACGGTGCAGCGGCTGCTGCCTGTGCT
GTGCCAGGCTCATGGACTGACACCAGAACAGGTGGTCGCAATCGCAAGCAACGGAGGTGGCAAG
CAGGCCCTGGAGACTGTGCAGGCCCTGCTTCCCGTGCTGTGCCAGGCTCACGGACTGACACCTC
AGCAGGTCGTCGCCATCGCCTCCAACAATGGTGGCAAGCAGGCCCTGGAGACAGTGCAGAGACT
GCTGCCAGTGCTGTGCCAAGCCCATGGACTGACACCACAGCAGGTCGTCGCTATCGCCTCTAAT
AACGGCGGCAAGCAGGCCCTGGAGACGGTACAGAGGCTGTTACCCGTGCTGTGCCAAGCACACG
GACTGACACCAGAGCAGGTCGTCGCAATCGCCAGCAATATCGGTGGCAAGCAGGCCCTGGAGAC
GGTCCAGCGCCTGCTCCCCGTGCTGTGCCAAGCCCACGGCCTGACCCCTCAGCAGGTCGTGGCT
ATTGCTAGCAATAACGGGGGCAAGCAGGCCCTGGAGACGGTTCAGCGGCTGTTGCCCGTGCTGT
GCCAAGCCCACGGTCTGACCCCTCAGCAGGTGGTCGCTATTGCTTCTAATGGAGGAGGCAAGCA
GGCCCTGGAGACGGTACAGAGACTGTTACCTGTGCTGTGCCAGGCACATGGCCTGACACCAGAG
CAGGTGGTCGCTATCGCCAGCAACATAGGTGGCAAGCAGGCCCTGGAGACGGTACAGAGGCTGC
TTCCCGTGCTGTGCCAAGCTCATGGCCTGACACCTGAACAGGTGGTCGCCATTGCTAGCAATAA
CGGTGGCAAGCAGGCCCTGGAGACGGTACAGCGGCTGTTACCAGTGCTGTGCCAAGCACATGGC
TTAACCCCTCAACAGGTCGTCGCAATTGCCTCTAATATCGGAGGCAAGCAGGCCCTGGAGACGG
TACAGCGGCTGCTCCCCGTGCTGTGCCAGGCGCACGGCCTGACTCCTCAGCAGGTCGTGGCAAT
CGCCAGCAACATCGGCGGCAGACCTGCCCTGGAGAGCATTGTGGCGCAGCTGAGCCGTCCAGAC
CCGGCCCTGGCGGCTCTGACCAATGATCACCTGGTGGCCCTGGCCTGCCTGGGTGGCCGTCCGG
CTCTGGATGCCGTGAAGAAAGGTCTGGGC
Translated amino acid sequence:
(SEQ ID NO: 317)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPQQVVAIASHDGGKQALETVORLLPVLCQAHGLTPEQVVAIASNGGGKQALE
TVQRLLPVLCQAHGLTPQQVVAIASNGGGKQALETVQRLLPVLCQAHGLTPEQVVAIASNGGGK
QALETVQALLPVLCQAHGLTPQQVVAIASNNGGKQALETVORLLPVLCQAHGLTPQQVVAIASN
NGGKQALETVQRLLPVLCQAHGLTPEQVVAIASNIGGKQALETVORLLPVLCQAHGLTPQQVVA
IASNNGGKQALETVQRLLPVLCQAHGLTPQQVVAIASNGGGKQALETVORLLPVLCQAHGLTPE
QVVAIASNIGGKQALETVQRLLPVLCQAHGLTPEQVVAIASNNGGKQALETVORLLPVLCQAHG
LTPQQVVAIASNIGGKQALETVORLLPVLCQAHGLTPQQVVAIASNIGGRPALESIVAQLSRPD
PALAALTNDHLVALACLGGRPALDAVKKGLG
(N) ND5.1-DdCBE Left mitoTALE repeat
(SEQ ID NO: 318)
CTGACCCCTGAGCAGGTGGTGGCCATCGCCAGCAATATCGGAGGCAAGCAGGCCCTGGAGACCG
TGCAGGCCCTGCTGCCCGTGCTGTGCCAGGCACACGGACTGACACCTCAGCAGGTCGTCGCCAT
CGCCTCCAACAATGGCGGCAAGCAGGCCCTGGAAACCGTGCAGAGGCTGTTACCCGTGCTGTGC
CAGGCCCACGGCCTGACACCCCAGCAGGTGGTGGCAATCGCATCTCACGATGGGGGCAAGCAGG
CCCTGGAGACGGTGCAGCGCCTGCTGCCTGTGCTGTGCCAGGCTCATGGACTGACACCAGAACA
GGTCGTGGCCATCGCCAGCAACATTGGCGGCAAGCAGGCCCTGGAGACTGTCCAGGCCCTGTTA
CCCGTGCTGTGCCAAGCCCATGGACTGACACCTGAACAGGTCGTGGCAATCGCATCCAATGGAG
GTGGCAAGCAGGCCCTGGAGACAGTGCAGGCCCTGCTGCCAGTGCTGTGCCAGGCTCACGGCCT
GACACCAGAACAGGTGGTCGCAATCGCATCTAATGGAGGAGGCAAGCAGGCCCTGGAGACGGTA
CAGGCCCTGTTGCCCGTGCTGTGCCAAGCCCACGGACTGACACCAGAGCAGGTCGTCGCTATTG
CTTCCAACATTGGAGGCAAGCAGGCCCTGGAGACGGTCCAGCGGCTGCTTCCCGTGCTGTGCCA
AGCTCATGGCCTGACACCAGAGCAGGTGGTCGCTATTGCCTCCAACAATGGAGGCAAGCAGGCC
CTGGAGACGGTTCAGGCCCTGCTTCCCGTGCTGTGCCAGGCTCATGGTCTGACACCCGAACAGG
TGGTCGCTATCGCCTCTCACGATGGAGGCAAGCAGGCCCTGGAGACGGTACAGAGGCTGTTACC
TGTGCTGTGCCAGGCCCATGGGCTGACCCCAGAACAGGTGGTCGCCATCGCCAGCAACATCGGC
GGCAAGCAGGCCCTGGAGACGGTACAGGCCCTGCTCCCCGTGCTGTGCCAAGCACATGGCCTGA
CACCCGAGCAGGTCGTGGCTATTGCTAGCAACAACGGGGGCAAGCAGGCCCTGGAGACGGTACA
GGCCCTGCTACCAGTGCTGTGCCAAGCGCACGGGCTGACCCCAGAGCAGGTCGTCGCAATCGCC
TCTAACAACGGTGGCAAGCAGGCCCTGGAGACGGTACAGGCCCTGCTGCCCGTGCTGTGCCAAG
CGCATGGGCTGACTCCAGAACAGGTGGTGGCTATCGCCAGCAACATTGGAGGCAAGCAGGCCCT
GGAGACGGTACAGCGGCTGCTACCCGTGCTGTGCCAAGCGCACGGTCTGACACCTCAGCAGGTG
GTCGCTATCGCTTCTAACATAGGGGGCAAGCAGGCCCTGGAGACGGTACAGCGGCTGCTGCCCG
TGCTGTGCCAAGCGCACGGACTGACCCCACAGCAGGTCGTCGCTATCGCCTCTAACGGAGGAGG
CAGACCCGCCCTGGAG
Translated amino acid sequence :
(SEQ ID NO: 319)
LTPEQVVAIASNIGGKQALETVQALLPVLCQAHGLTPQQVVAIASNNGGKQALETVORLLPVLC
QAHGLTPQQVVAIASHDGGKQALETVQRLLPVLCQAHGLTPEQVVAIASNIGGKQALETVQALL
PVLCQAHGLTPEQVVAIASNGGGKQALETVQALLPVLCQAHGLTPEQVVAIASNGGGKQALETV
QALLPVLCQAHGLTPEQVVAIASNIGGKQALETVORLLPVLCQAHGLTPEQVVAIASNNGGKQA
LETVQALLPVLCQAHGLTPEQVVAIASHDGGKQALETVORLLPVLCQAHGLTPEQVVAIASNIG
GKQALETVQALLPVLCQAHGLTPEQVVAIASNNGGKQALETVQALLPVLCQAHGLTPEQVVAIA
SNNGGKQALETVQALLPVLCQAHGLTPEQVVAIASNIGGKQALETVORLLPVLCQAHGLTPQQV
VAIASNIGGKQALETVORLLPVLCQAHGLTPQQVVAIASNGGGRPALE
(O) ND5.2-DdCBE Right mitoTALE repeat
(SEQ ID NO: 320)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACAT
CGTGGCCCTGAGCCAGCACCCAGCCGCGCTGGGCACCGTGGCCGTGAAATATCAGGACATGATT
GCTGCCCTGCCAGAGGCCACCCATGAAGCTATTGTGGGCGTGGGCAAGCAGTGGAGCGGTGCTC
GTGCGCTGGAGGCGCTGCTGACCGTGGCTGGTGAACTGCGTGGTCCGCCGCTGCAGCTGGATAC
CGGTCAGCTGCTGAAAATTGCCAAACGTGGCGGTGTGACCGCGGTGGAAGCCGTGCATGCTTGG
CGTAATGCTCTGACCGGTGCGCCGCTGAACCTGACCCCGCAGCAGGTGGTGGCTATTGCCAGCA
ACAACGGCGGTAAACAGGCTCTGGAGACCGTGCAGCGTCTGCTGCCGGTGCTGTGCCAGGCTCA
TGGTCTGACCCCGGAGCAGGTGGTGGCCATTGCTAGCCATGATGGCGGCAAGCAGGCGCTGGAA
ACCGTGCAGCGCCTGTTACCGGTGCTGTGCCAAGCCCATGGTCTGACCCCGCAGCAAGTIGTGG
CTATTGCGAGCAACGGCGGTGGCAAACAGGCCCTGGAAACCGTTCAGCGTCTGTTACCGGTGCT
GTGCCAGGCCCATGGCCTGACCCCGGAACAAGTGGTGGCTATCGCCAGCAACATTGGTGGCAAG
CAGGCCCTGGAAACCGTGCAGGCGCTGTTGCCGGTGCTGTGCCAAGCCCATGGGCTGACCCCGC
AGCAAGTGGTTGCGATCGCTAGCAACAACGGTGGCAAACAGGCTCTGGAAACCGTTCAGCGCCT
GCTTCCGGTGCTGTGCCAAGCGCATGGCTTAACCCCGCAGCAAGTTGTGGCCATTGCGAGCAAC
AACGGTGGCAAGCAGGCGCTGGAGACCGTTCAGCGTCTGCTTCCGGTGCTGTGCCAGGCGCATG
GCCTGACCCCGGAGCAAGTGGTGGCTATTGCTAGCCACGATGGTGGCAAACAGGCCCTGGAGAC
CGTGCAGCGCCTGCTCCCGGTGCTGTGCCAGGCCCATGGATTAACCCCGCAGCAAGTGGTGGCC
ATCGCCAGCAATGGCGGCGGCAAGCAGGCTCTGGAAACTGTGCAGCGTCTGTTACCGGTGCTGT
GCCAGGCCCATGGGTTAACCCCGCAGCAGGTTGTTGCCATTGCCTCCAATAATGGCGGTAAACA
GGCGCTGGAGACTGTGCAGCGCCTGCTACCGGTGCTGTGCCAGGCACATGGTCTGACCCCGGAA
CAAGTTGTTGCCATTGCGTCCCATGATGGCGGCAAGCAGGCCCTGGAGACTGTTCAGCGTCTGC
TCCCGGTGCTGTGCCAGGCCCATGGTTTAACCCCGGAACAAGTTGTGGCCATTGCTAGCCACGA
TGGCGGTAAACAGGCTCTGGAAACTGTTCAGCGCCTGCTGCCGGTGCTGTGCCAAGCACATGGC
TTAACCCCGGAACAGGTTGTTGCTATTGCCAGCAACATCGGCGGCAAGCAGGCTCTGGAGACCG
TTCAGGCCCTGTTGCCGGTGCTGTGCCAGGCCCATGGGCTTACCCCGGAACAAGTGGTTGCCAT
CGCCAGCAACATTGGCGGTAAACAGGCGCTGGAAACCGTTCAGGCTCTGTTGCCGGTGCTGTGC
CAGGCTCATGGCCTTACCCCGCAGCAAGTIGTGGCGATTGCTAGCAATGGCGGTGGCAAGCAGG
CGCTGGAGACGGTTCAGCGTCTGCTACCGGTGCTGTGCCAGGCTCATGGATTGACCCCGCAGCA
GGTCGTGGCCATTGCCTCCAATAACGGTGGCAAACAGGCGCTGGAGACAGTTCAGCGCCTGCTG
CCGGTGCTGTGCCAGGCTCATGGGTTGACCCCGCAGCAGGTAGTTGCTATTGCTAGCAATGGTG
GCGGTCGTCCGGCCCTGGAGAGCATTGTGGCGCAGCTGAGCCGTCCAGACCCGGCGCTGGCGGC
TCTGACCAACGATCACCTGGTGGCGCTGGCTTGCCTGGGCGGTCGTCCGGCCCTGGATGCCGTG
AAGAAAGGCCTGGGT
Translated amino acid sequence:
(SEQ ID NO: 321)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPQQVVAIASNNGGKQALETVORLLPVLCQAHGLTPEQVVAIASHDGGKQALE
TVQRLLPVLCQAHGLTPQQVVAIASNGGGKQALETVORLLPVLCQAHGLTPEQVVAIASNIGGK
QALETVQALLPVLCQAHGLTPQQVVAIASNNGGKQALETVQRLLPVLCQAHGLTPQQVVAIASN
NGGKQALETVORLLPVLCQAHGLTPEQVVAIASHDGGKQALETVORLLPVLCQAHGLTPQQVVA
IASNGGGKQALETVORLLPVLCQAHGLTPQQVVAIASNNGGKQALETVORLLPVLCQAHGLTPE
QVVAIASHDGGKQALETVORLLPVLCQAHGLTPEQVVAIASHDGGKQALETVORLLPVLCQAHG
LTPEQVVAIASNIGGKQALETVQALLPVLCQAHGLTPEQVVAIASNIGGKQALETVQALLPVLC
QAHGLTPQQVVAIASNGGGKQALETVQRLLPVLCQAHGLTPQQVVAIASNNGGKQALETVORLL
PVLCQAHGLTPQQVVAIASNGGGRPALESIVAQLSRPDPALAALTNDHLVALACLGGRPALDAV
KKGLG
(P) ND5.2-DdCBE Left mitoTALE repeat
(SEQ ID NO: 322)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACAT
TGTGGCCCTGAGCCAGCACCCAGCCGCGCTGGGCACCGTGGCCGTGAAATATCAGGATATGATT
GCTGCCCTGCCAGAGGCCACCCATGAAGCTATTGTGGGCGTGGGCAAGCAGTGGAGCGGTGCTC
GTGCGCTGGAGGCGCTGCTGACCGTGGCTGGTGAACTGCGTGGTCCGCCGCTGCAGCTGGACAC
CGGTCAGCTGCTGAAAATCGCGAAACGTGGCGGTGTGACCGCGGTGGAAGCCGTGCATGCTTGG
CGTAATGCTCTGACCGGTGCGCCGCTGAACCTGACCCCTGAGCAGGTGGTGGCAATCGCAAGCC
ACGACGGAGGCAAGCAGGCCCTGGAGACAGTGCAGGCCCTGCTGCCCGTGCTGTGCCAGGCACA
CGGCCTGACACCTGAGCAGGTGGTGGCCATCGCCTCCAACATCGGCGGCAAGCAGGCCCTGGAG
ACAGTACAGAGGCTGTTACCCGTGCTGTGCCAGGCCCACGGCCTGACACCCCAGCAGGTCGTCG
CCATCGCCTCTAATATTGGAGGCAAGCAGGCCCTGGAGACAGTCCAGCGCCTGCTGCCTGTGCT
GTGCCAGGCTCATGGCCTGACACCAGAACAGGTCGTGGCCATCGCCAGTAATATTGGGGGCAAG
CAGGCCCTGGAGACAGTTCAGGCCCTGTTACCCGTGCTGTGCCAAGCCCATGGCCTGACACCTG
AACAGGTGGTCGCCATCGCCTCCAATATTGGTGGCAAGCAGGCCCTGGAGACAGTACAGGCCCT
GCTGCCAGTGCTGTGCCAGGCTCACGGCCTGACACCAGAGCAGGTCGTCGCAATCGCATCTCAT
GATGGCGGCAAGCAGGCCCTGGAGACAGTACAGGCCCTGTTACCCGTGCTGTGCCAAGCGCACG
GCCTGACCCCTGAACAGGTCGTGGCTATTGCAAGCCACGATGGTGGCAAGCAGGCCCTGGAGAC
AGTACAGCGGCTGCTTCCCGTGCTGTGCCAAGCTCATGGCCTGACACCTGAGCAGGTCGTCGCT
ATTGCTAGCAATATTGGCGGCAAGCAGGCCCTGGAGACAGTACAGGCCCTGCTCCCCGTGCTGT
GCCAAGCACACGGCCTGACACCCGAACAGGTGGTGGCTATCGCCTCTAATGGAGGTGGCAAGCA
GGCCCTGGAGACAGTACAGAGGCTGCTTCCTGTGCTGTGCCAGGCCCATGGCCTGACCCCTGAG
CAGGTCGTGGCTATTGCCAGTAATATAGGAGGCAAGCAGGCCCTGGAGACAGTACAGGCCCTGC
TACCCGTGCTGTGCCAAGCGCATGGCCTGACCCCAGAACAGGTCGTGGCAATCGCATCTCATGA
CGGCGGCAAGCAGGCCCTGGAGACAGTACAGGCCCTGCTACCAGTGCTGTGCCAAGCACATGGC
CTGACCCCCGAACAGGTGGTGGCAATCGCCTCTCACGACGGGGGCAAGCAGGCCCTGGAGACAG
TACAGGCCCTGCTACCCGTGCTGTGCCAAGCGCACGGCCTGACGCCAGAACAGGTGGTCGCTAT
CGCAAGCAACGGCGGTGGCAAGCAGGCCCTGGAGACAGTACAGCGGCTGCTACCCGTGCTGTGC
CAAGCGCACGGCCTGACTCCTCAGCAGGTCGTCGCTATCGCATCTCATGATGGTGGCAAGCAGG
CCCTGGAGACAGTACAGCGGCTGCTACCCGTGCTGTGCCAAGCGCACGGCCTGACACCACAGCA
GGTCGTCGCAATTGCATCTAACGGAGGAGGCAGACCCGCCCTGGAGAGCATTGTGGCTCAGCTG
AGCCGTCCAGACCCGGCCCTGGCGGCTCTGACCAACGATCACCTGGTGGCGCTGGCTTGCCTGG
GCGGTCGTCCGGCCCTGGATGCGGTGAAGAAAGGCCTGGGT
Translated amino acid sequence:
(SEQ ID NO: 323)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLQLDTGQLLKIAKRGGVTAVEAVHAW
RNALTGAPLNLTPEQVVAIASHDGGKQALETVQALLPVLCQAHGLTPEQVVAIASNIGGKQALE
TVQRLLPVLCQAHGLTPQQVVAIASNIGGKQALETVORLLPVLCQAHGLTPEQVVAIASNIGGK
QALETVQALLPVLCQAHGLTPEQVVAIASNIGGKQALETVQALLPVLCQAHGLTPEQVVAIASH
DGGKQALETVQALLPVLCQAHGLTPEQVVAIASHDGGKQALETVORLLPVLCQAHGLTPEQVVA
IASNIGGKQALETVQALLPVLCQAHGLTPEQVVAIASNGGGKQALETVORLLPVLCQAHGLTPE
QVVAIASNIGGKQALETVQALLPVLCQAHGLTPEQVVAIASHDGGKQALETVQALLPVLCQAHG
LTPEQVVAIASHDGGKOALETVQALLPVLCQAHGLTPEQVVAIASNGGGKQALETVORLLPVLC
QAHGLTPQQVVAIASHDGGKQALETVORLLPVLCQAHGLTPQQVVAIASNGGGRPALESIVAQL
SRPDPALAALTNDHLVALACLGGRPALDAVKKGLG
(Q) ND5.3-DdCBE Right mitoTALE repeat
(SEQ ID NO: 324)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACAT
CGTGGCTCTGAGCCAGCACCCAGCCGCGCTGGGTACCGTGGCCGTGAAATATCAGGACATGATT
GCTGCCCTGCCAGAGGCTACCCATGAAGCGATTGTGGGTGTGGGCAAGCAGTGGAGCGGTGCTC
GTGCGCTGGAGGCCCTGCTGACCGTGGCCGGTGAACTGCGTGGCCCGCCGCTGCAGCTGGATAC
CGGCCAGCTGCTGAAAATCGCGAAACGTGGCGGTGTGACCGCTGTGGAAGCTGTGCATGCCTGG
CGTAATGCTCTGACCGGTGCCCCGCTGAACCTGACACCACAGCAGGTCGTCGCTATCGCTTCAA
ACATTGGGGGGAAACAGGCACTGGAAACCGTCCAGAGACTGCTGCCCGTCCTGTGCCAGGCCCA
CGGCCTGACCCCTGAGCAGGTGGTGGCCATCGCCAGCAATATCGGAGGCAAGCAGGCCCTGGAG
ACCGTGCAGCGGCTGCTGCCCGTGCTGTGCCAAGCCCACGGCTTAACACCTCAGCAGGTCGTGG
CTATCGCCTCCAACAATGGCGGCAAGCAGGCCCTGGAGACGGTGCAGAGACTGCTGCCAGTGCT
GTGCCAGGCCCACGGCTTAACACCAGAACAGGTCGTGGCCATCGCCTCTAACATTGGCGGCAAG
CAGGCCCTGGAGACTGTGCAGGCCCTGCTGCCCGTGCTGTGCCAGGCCCACGGCCTTACACCAC
AGCAGGTGGTGGCAATCGCCAGCAATGGAGGGGGCAAGCAGGCCCTGGAGACAGTGCAGAGGCT
GCTGCCCGTGCTGTGCCAAGCCCACGGCCTGACACCTCAGCAGGTGGTCGCCATCGCCTCCAAC
GGAGGTGGCAAGCAGGCCCTGGAGACGGTACAGCGCCTGCTGCCCGTGCTGTGCCAAGCCCACG
GCCTAACACCCGAACAGGTCGTCGCCATCGCCTCTAACATCGGCGGCAAGCAGGCCCTGGAGAC
GGTCCAGCGGCTGCTGCCTGTGCTGTGCCAAGCCCACGGCCTTACCCCTCAGCAGGTCGTGGCA
ATCGCCAGCAACAATGGTGGCAAGCAGGCCCTGGAGACGGTTCAGAGACTGCTGCCCGTGCTGT
GCCAAGCCCACGGCCTCACACCTCAGCAGGTGGTGGCCATTGCCTCCAACGGAGGAGGCAAGCA
GGCCCTGGAGACGGTACAGAGGCTGCTGCCAGTGCTGTGCCAGGCCCACGGCCTAACACCAGAA
CAGGTGGTCGCTATTGCCTCTAACATTGGTGGCAAGCAGGCCCTGGAGACGGTACAGCGCCTGC
TGCCCGTGCTGTGCCAAGCCCACGGCCTAACGCCAGAACAGGTCGTCGCTATCGCCAGCAACGG
AGGAGGCAAGCAGGCCCTGGAGACGGTACAGCGGCTGCTGCCCGTGCTGTGCCAAGCCCACGGC
CTAACCCCACAGCAGGTCGTGGCCATTGCCTCCAATAACGGCGGCAAGCAGGCCCTGGAGACGG
TACAGCGGCTGCTGCCCGTGCTGTGCCAAGCCCACGGCCTAACTCCCCAGCAAGTCGTCGCTAT
TGCCTCTAATAACGGGGGCAAGCAGGCCCTGGAGACGGTACAGAGACTGCTGCCCGTGCTGTGC
CAAGCCCACGGCCTGACACCACAGCAGGTCGTCGCCATCGCAAGCAACGGAGGAGGGAGGCCCG
CACTGGAGAGCATTGTGGCGCAGCTGAGCCGTCCAGACCCGGCCCTGGCGGCTCTGACCAATGA
TCACCTGGTGGCCCTGGCCTGCCTGGGTGGCCGTCCGGCTCTGGATGCCGTGAAGAAAGGTCTG
GGC
Translated amino acid sequence:
(SEQ ID NO: 325)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPQQVVAIASNIGGKQALETVORLLPVLCQAHGLTPEQVVAIASNIGGKQALE
TVQRLLPVLCQAHGLTPQQVVAIASNNGGKQALETVORLLPVLCQAHGLTPEQVVAIASNIGGK
QALETVQALLPVLCQAHGLTPQQVVAIASNGGGKQALETVORLLPVLCQAHGLTPQQVVAIASN
GGGKQALETVQRLLPVLCQAHGLTPEQVVAIASNIGGKQALETVORLLPVLCQAHGLTPQQVVA
IASNNGGKQALETVORLLPVLCQAHGLTPQQVVAIASNGGGKQALETVORLLPVLCQAHGLTPE
QVVAIASNIGGKQALETVORLLPVLCQAHGLTPEQVVAIASNGGGKQALETVORLLPVLCQAHG
LTPQQVVAIASNNGGKQALETVORLLPVLCQAHGLTPQQVVAIASNNGGKQALETVORLLPVLC
QAHGLTPQQVVAIASNGGGRPALESIVAQLSRPDPALAALINDHLVALACLGGRPALDAVKKGL
G
(R) ND5.3-DdCBE Left mitoTALE repeat
(SEQ ID NO: 326)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACAT
TGTGGCCCTGAGCCAGCACCCAGCCGCGCTGGGCACCGTGGCCGTGAAATATCAGGATATGATT
GCTGCCCTGCCAGAGGCCACCCATGAAGCTATTGTGGGCGTGGGCAAGCAGTGGAGCGGTGCTC
GTGCGCTGGAGGCGCTGCTGACCGTGGCTGGTGAACTGCGTGGTCCGCCGCTGCAGCTGGACAC
CGGTCAGCTGCTGAAAATCGCGAAACGTGGCGGTGTGACCGCGGTGGAAGCCGTGCATGCTTGG
CGTAATGCTCTGACCGGTGCGCCGCTGAACCTGACTCCCGAACAGGTGGTCGCTATCGCTTCTC
ATGATGGCGGAAAACAGGCTCTGGAAACCGTCCAGGCTCTGCTGCCCGTGCTGTGCCAGGCCCA
CGGCCTGACCCCACAGCAGGTCGTCGCAATCGCCAGCAATATCGGAGGCAAGCAGGCCCTGGAG
ACCGTGCAGCGGCTGCTGCCCGTGCTGTGCCAAGCCCACGGCTTAACACCTCAGCAGGTGGTGG
CCATCGCCTCCAACAATGGCGGCAAGCAGGCCCTGGAGACGGTGCAGAGACTGCTGCCAGTGCT
GTGCCAGGCCCACGGCTTAACACCAGAACAGGTCGTGGCAATCGCCTCTAACGGAGGGGGCAAG
CAGGCCCTGGAGACTGTGCAGGCCCTGCTGCCCGTGCTGTGCCAGGCCCACGGCCTTACACCAG
AACAGGTGGTCGCCATTGCCAGCAATGGAGGTGGCAAGCAGGCCCTGGAGACAGTCCAGGCCCT
GCTGCCCGTGCTGTGCCAAGCCCACGGCCTGACACCTGAACAGGTGGTCGCAATCGCCTCCCAC
GATGGGGGCAAGCAGGCCCTGGAGACGGTACAGGCCCTGCTGCCCGTGCTGTGCCAAGCCCACG
GCCTAACACCCGAACAGGTGGTGGCCATTGCCTCTAACGGAGGAGGCAAGCAGGCCCTGGAGAC
GGTCCAGCGGCTGCTGCCTGTGCTGTGCCAAGCCCACGGCCTTACCCCTGAACAAGTCGTGGCC
ATCGCCAGCAATGGAGGAGGCAAGCAGGCCCTGGAGACGGTTCAGGCCCTGCTGCCCGTGCTGT
GCCAAGCCCACGGCCTCACACCTGAACAAGTIGTGGCCATCGCCTCCCACGATGGTGGCAAGCA
GGCCCTGGAGACGGTACAGAGGCTGCTGCCAGTGCTGTGCCAGGCCCACGGCCTAACACCAGAA
CAGGTGGTGGCTATCGCCTCTAACATTGGCGGCAAGCAGGCCCTGGAGACGGTACAGGCCCTGC
TGCCCGTGCTGTGCCAAGCCCACGGCCTAACGCCAGAACAGGTCGTCGCTATTGCCAGCAACAT
TGGGGGCAAGCAGGCCCTGGAGACGGTACAGGCCCTGCTGCCCGTGCTGTGCCAAGCCCACGGC
CTAACCCCTGAACAGGTGGTGGCAATCGCCTCCAACATTGGTGGCAAGCAGGCCCTGGAGACGG
TACAGGCCCTGCTGCCCGTGCTGTGCCAAGCCCACGGCCTAACTCCCGAGCAGGTCGTCGCCAT
CGCCTCTAATGGCGGCGGCAAGCAGGCCCTGGAGACGGTACAGAGGCTGCTGCCTGTGCTGTGC
CAAGCCCACGGCCTAACGCCGCAGCAAGTCGTCGCTATTGCCAGCAATATTGGCGGCAAGCAGG
CCCTGGAGACGGTACAGCGCCTGCTGCCCGTGCTGTGCCAAGCCCACGGCCTGACCCCCCAGCA
GGTGGTGGCAATCGCTTCAAACGGAGGAGGGAGACCCGCTCTGGAAAGCATTGTGGCTCAGCTG
AGCCGTCCAGACCCGGCCCTGGCGGCTCTGACCAACGATCACCTGGTGGCGCTGGCTTGCCTGG
GCGGTCGTCCGGCCCTGGATGCGGTGAAGAAAGGCCTGGGT
Translated amino acid sequence:
(SEQ ID NO: 327)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGQLLKIAKRGGVTAVEAVHAW
RNALTGAPLNLTPEQVVAIASHDGGKQALETVQALLPVLCQAHGLTPQQVVAIASNIGGKQALE
TVQRLLPVLCQAHGLTPQQVVAIASNNGGKQALETVORLLPVLCQAHGLTPEQVVAIASNGGGK
QALETVQALLPVLCQAHGLTPEQVVAIASNGGGKQALETVQALLPVLCQAHGLTPEQVVAIASH
DGGKQALETVQALLPVLCQAHGLTPEQVVAIASNGGGKQALETVORLLPVLCQAHGLTPEQVVA
IASNGGGKQALETVQALLPVLCQAHGLTPEQVVAIASHDGGKQALETVQRLLPVLCQAHGLTPE
QVVAIASNIGGKQALETVQALLPVLCQAHGLTPEQVVAIASNIGGKQALETVQALLPVLCQAHG
LTPEQVVAIASNIGGKQALETVOALLPVLCQAHGLTPEQVVAIASNGGGKQALETVQRLLPVLC
QAHGLTPQQVVAIASNIGGKQALETVORLLPVLCQAHGLTPQQVVAIASNGGGRPALESIVAQL
SRPDPALAALTNDHLVALACLGGRPALDAVKKGLG
(S) ATP8-DdCBE Right mitoTALE repeat
(SEQ ID NO: 328)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACAT
CGTGGCTCTGAGCCAGCACCCAGCCGCGCTGGGTACCGTGGCCGTGAAATATCAGGACATGATT
GCTGCCCTGCCAGAGGCTACCCATGAAGCGATTGTGGGTGTGGGCAAGCAGTGGAGCGGTGCTC
GTGCGCTGGAGGCCCTGCTGACCGTGGCCGGTGAACTGCGTGGCCCGCCGCTGCAGCTGGATAC
CGGCCAGCTGCTGAAAATCGCGAAACGTGGCGGTGTGACCGCTGTGGAAGCTGTGCATGCCTGG
CGTAATGCTCTGACCGGTGCCCCGCTGAACCTGACCCCTCAGCAGGTGGTGGCCATCGCCAGCA
ACATCGGCGGCAAGCAGGCCCTGGAGACAGTGCAGAGGCTGCTGCCCGTGCTGTGCCAGGCACA
CGGCCTGACACCTGAGCAGGTGGTGGCAATCGCATCCAATGGAGGAGGCAAGCAGGCCCTGGAG
ACAGTACAGCGCCTGTTACCCGTGCTGTGCCAGGCCCACGGCCTGACACCCCAGCAGGTCGTCG
CCATCGCCTCTAACAATGGGGGCAAGCAGGCCCTGGAGACAGTCCAGCGGCTGCTGCCTGTGCT
GTGCCAGGCTCATGGCCTGACACCAGAACAGGTCGTGGCTATTGCCAGCAACAATGGTGGCAAG
CAGGCCCTGGAGACAGTTCAGGCCCTGCTTCCCGTGCTGTGCCAGGCTCACGGCCTGACACCAC
AGCAGGTCGTGGCCATCGCCTCCAACAATGGCGGCAAGCAGGCCCTGGAGACAGTACAGAGACT
GCTGCCAGTGCTGTGCCAAGCCCATGGCCTGACCCCTCAGCAGGTCGTGGCAATCGCATCTCAC
GACGGTGGCAAGCAGGCCCTGGAGACAGTACAGAGGCTGTTACCCGTGCTGTGCCAAGCACACG
GCCTGACACCAGAGCAGGTCGTCGCAATCGCAAGCAACGGCGGCGGCAAGCAGGCCCTGGAGAC
AGTACAGCGCCTGCTCCCCGTGCTGTGCCAAGCCCACGGCCTGACACCTCAGCAGGTGGTCGCC
ATTGCCAGCAACGGCGGGGGCAAGCAGGCCCTGGAGACAGTACAGCGGCTGTTGCCCGTGCTGT
GCCAAGCCCACGGCCTGACGCCCCAGCAGGTGGTCGCCATCGCATCTAACGGCGGTGGCAAGCA
GGCCCTGGAGACAGTACAGCGGCTGCTTCCTGTGCTGTGCCAGGCCCATGGCCTGACCCCCGAA
CAGGTCGTGGCTATCGCTAGCAACAATGGCGGCAAGCAGGCCCTGGAGACAGTACAGAGACTGT
TACCCGTGCTGTGCCAAGCGCATGGCCTGACCCCTGAACAGGTCGTGGCAATTGCCTCCAATAA
CGGTGGCAAGCAGGCCCTGGAGACAGTACAGCGGCTGCTACCAGTGCTGTGCCAAGCACATGGC
CTGACCCCCCAGCAGGTCGTGGCTATTGCATCTAATGGAGGAGGCAGACCCGCCCTGGAGAGCA
TTGTGGCGCAGCTGAGCCGTCCAGACCCGGCCCTGGCGGCTCTGACCAATGATCACCTGGTGGC
CCTGGCCTGCCTGGGTGGCCGTCCGGCTCTGGATGCCGTGAAGAAAGGTCTGGGC
Translated amino acid sequence:
(SEQ ID NO: 329)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGQLLKIAKRGGVTAVEAVHAW
RNALTGAPLNLTPQQVVAIASNIGGKQALETVORLLPVLCQAHGLTPEQVVAIASNGGGKQALE
TVQRLLPVLCQAHGLTPQQVVAIASNNGGKQALETVORLLPVLCQAHGLTPEQVVAIASNNGGK
QALETVQALLPVLCQAHGLTPQQVVAIASNNGGKQALETVORLLPVLCQAHGLTPQQVVAIASH
DGGKQALETVORLLPVLCQAHGLTPEQVVAIASNGGGKQALETVORLLPVLCQAHGLTPQQVVA
IASNGGGKQALETVORLLPVLCQAHGLTPQQVVAIASNGGGKQALETVORLLPVLCQAHGLTPE
QVVAIASNNGGKQALETVORLLPVLCQAHGLTPEQVVAIASNNGGKQALETVORLLPVLCQAHG
LTPQQVVAIASNGGGRPALESIVAQLSRPDPALAALTNDHLVALACLGGRPALDAVKKGLG
(T) ATP8-DdCBE Left mitoTALE repeat
(SEQ ID NO: 330)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACAT
CGTGGCCCTGAGCCAGCACCCAGCCGCGCTGGGCACCGTGGCCGTGAAATATCAGGACATGATT
GCTGCCCTGCCAGAGGCCACCCATGAAGCTATTGTGGGCGTGGGCAAGCAGTGGAGCGGTGCTC
GTGCGCTGGAGGCGCTGCTGACCGTGGCTGGTGAACTGCGTGGTCCGCCGCTGCAGCTGGATAC
CGGTCAGCTGCTGAAAATCGCGAAACGTGGCGGTGTGACCGCGGTGGAAGCCGTGCATGCTTGG
CGTAATGCTCTGACCGGTGCGCCGCTGAACCTGACCCCGGAGCAGGTGGTGGCTATCGCCAGCA
ACATTGGCGGTAAACAGGCCCTGGAAACCGTGCAGGCGCTGCTGCCGGTGCTGTGCCAGGCTCA
TGGTCTGACCCCGCAGCAGGTGGTGGCGATCGCTAGCAACGGCGGTGGCAAGCAGGCTCTGGAG
ACCGTGCAGCGTCTGTTACCGGTGCTGTGCCAAGCCCATGGCCTGACCCCGCAGCAAGTIGTGG
CCATTGCGAGCAATGGTGGCGGTAAACAGGCGCTGGAAACCGTGCAGCGCCTGTTGCCGGTGCT
GTGCCAAGCCCATGGGCTGACCCCGGAACAAGTTGTTGCTATCGCCAGCAACATCGGCGGCAAG
CAGGCTCTGGAAACCGTGCAGGCCCTGCTTCCGGTGCTGTGCCAAGCGCATGGTCTGACCCCGG
AACAAGTGGTGGCCATCGCTTCCAATATTGGCGGTAAACAGGCGCTGGAGACCGTGCAGGCTCT
GCTCCCGGTGCTGTGCCAAGCACATGGTCTGACCCCGGAGCAAGTIGTGGCTATTGCCTCCAAT
ATCGGCGGCAAGCAGGCCCTGGAGACCGTTCAGGCGCTGTTACCGGTGCTGTGCCAGGCCCATG
GATTAACCCCGGAGCAAGTGGTGGCTATTGCTAGCCATGATGGCGGTAAACAGGCCCTGGAGAC
TGTTCAGCGTCTGCTACCGGTGCTGTGCCAGGCCCATGGTTTAACCCCGGAACAGGTTGTTGCC
ATCGCTTCCAACATCGGCGGCAAGCAGGCTCTGGAAACGGTGCAGGCCCTGTTACCGGTGCTGT
GCCAGGCCCATGGGTTAACCCCGGAACAAGTTGTGGCCATTGCCTCCCATGACGGCGGTAAACA
GGCTCTGGAGACCGTTCAGCGCCTGCTACCGGTGCTGTGCCAGGCGCATGGCTTAACCCCGGAA
CAAGTGGTTGCCATTGCGTCCAATATCGGCGGCAAGCAGGCGCTGGAGACCGTTCAGGCTCTGC
TTCCGGTGCTGTGCCAGGCACATGGCCTTACCCCGGAACAAGTGGTCGCGATCGCTTCCAACAT
TGGCGGTAAACAGGCCCTGGAAACGGTTCAGGCGCTGCTTCCGGTGCTGTGCCAGGCCCATGGG
CTTACCCCGGAACAGGTTGTGGCTATTGCCAGTAATATCGGCGGCAAGCAGGCTCTGGAAACTG
TGCAGGCCCTGCTACCGGTGCTGTGCCAGGCTCATGGGCTGACCCCGGAGCAAGTGGTTGCCAT
TGCCTCCCATGATGGCGGTAAACAGGCGCTGGAAACGGTGCAGCGTCTGCTTCCGGTGCTGTGC
CAGGCTCATGGCTTAACCCCGCAGCAAGTTGTTGCGATTGCTAGCAATGGCGGTGGCAAGCAGG
CCCTGGAAACTGTTCAGCGCCTGTTACCGGTGCTGTGCCAGGCCCATGGGCTAACCCCGGAACA
GGTGGTTGCTATTGCCAGCAACATTGGTGGCAAACAGGCGCTGGAAACTGTGCAGGCTCTGCTT
CCGGTGCTGTGCCAGGCCCATGGGCTGACCCCGCAGCAAGTGGTTGCTATTGCTAGCAATGGTG
GCGGTCGTCCGGCCCTGGAGAGCATTGTGGCGCAGCTGAGCCGTCCAGACCCGGCCCTGGCGGC
TCTGACCAACGATCACCTGGTGGCGCTGGCTTGCCTGGGCGGTCGTCCGGCCCTGGATGCGGTG
AAGAAAGGCCTGGGT
Translated amino acid sequence:
(SEQ ID NO: 331)
DIADLRTLGYSQQQQEKIKPKVRSTVAQHHEALVGHGFTHAHIVALSQHPAALGTVAVKYQDMI
AALPEATHEAIVGVGKOWSGARALEALLTVAGELRGPPLOLDTGOLLKIAKRGGVTAVEAVHAW
RNALTGAPLNLTPEQVVAIASNIGGKQALETVQALLPVLCQAHGLTPQQVVAIASNGGGKQALE
TVQRLLPVLCQAHGLTPQQVVAIASNGGGKQALETVORLLPVLCQAHGLTPEQVVAIASNIGGK
QALETVQALLPVLCQAHGLTPEQVVAIASNIGGKQALETVQALLPVLCQAHGLTPEQVVAIASN
IGGKQALETVQALLPVLCQAHGLTPEQVVAIASHDGGKOALETVORLLPVLCQAHGLTPEQVVA
IASNIGGKQALETVQALLPVLCQAHGLTPEQVVAIASHDGGKQALETVORLLPVLCQAHGLTPE
QVVAIASNIGGKQALETVQALLPVLCQAHGLTPEQVVAIASNIGGKQALETVQALLPVLCQAHG
LTPEQVVAIASNIGGKQALETVQALLPVLCQAHGLTPEQVVAIASHDGGKQALETVQRLLPVLC
QAHGLTPQQVVAIASNGGGKQALETVORLLPVLCQAHGLTPEQVVAIASNIGGKQALETVQALL
PVLCQAHGLTPQQVVAIASNGGGRPALESIVAQLSRPDPALAALTNDHLVALACLGGRPALDAV
KKGLG
Intact DddAtox-Cas9 fusions sequences
All intact DddAtox-Cas9 have the general architecture of (from
N- to C-terminus): bpNLS-DddAtox-linker 1-dSpCas9 or
SpCas9(D10A)-10aa linker--10aa linker- UGI-4aa linker-bpNLS
DddAtox
(SEQ IN NO: 338)
GSYALGPYQISAPOLPAYNGQTVGTFYYVNDAGGLESKVFSSGGPTPYPNYANAGHVEGQSALF
MRDNGISEGLVFHNNPEGTCGFCVNMTETLLPENAKMTVVPPEGAIPVKRGATGETKVFTGNSN
SPKSPTKGGC
dSpCas9
(SEQ ID NO: 339)
DKKYSIGLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNED
LAEDAKLQLSKDTYDDDLDNLLAQIGDQYADLFLAAKNLSDAILLSDILRVNTEITKAPLSASM
IKRYDEHHQDLTLLKALVROQLPEKYKEIFFDQSKNGYAGYIDGGASQEEFYKFIKPILEKMDG
TEELLVKLNREDLLRKORTFDNGSIPHOIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKOLKEDYFKKIECFDS
VEISGVEDRFNASLGTYHDLLKIIKDKDFLDNEENEDILEDIVLTLTLFEDREMIEERLKTYAH
LFDDKVMKOLKRRRYTGWGRLSRKLINGIRDKQSGKTILDFLKSDGFANRNFMQLIHDDSLTFK
EDIQKAQVSGOGDSLHEHIANLAGSPAIKKGILQTVKVVDELVKVMGRHKPENIVIEMARENQT
TOKGOKNSRERMKRIEEGIKELGSQILKEHPVENTQLQNEKLYLYYLONGRDMYVDQELDINRL
SDYDVDAIVPQSFLKDDSIDNKVLTRSDKNRGKSDNVPSEEVVKKMKNYWROLLNAKLITORKF
DNLTKAERGGLSELDKAGFIKROLVETROITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKOLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGD
SpCas9 (D10A)
(SEQ ID NO: 340)
DKKYSIGLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNED
LAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDG
TEELLVKLNREDLLRKORTFDNGSIPHOIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKOLKEDYFKKIECFDS
VEISGVEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMARENOT
TQKGOKNSRERMKRIEEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNYWRQLLNAKLITORKF
DNLTKAERGGLSELDKAGFIKRQLVETROITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKOLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGD
UGI
(SEQ ID NO: 341)
TNLSDIIEKETGKOLVIQESILMLPEEVEEVIGNKPESDILVHTAYDESTDENVMLLTSDAPEY
KPWALVIQDSNGENKIKML
bpNLS
(SEQ ID NO: 342)
KRTADGSEFEPKKKRKV
Linker 1: 32aa linker
(SEQ ID NO: 343)
SGGSSGGSSGSETPGTSESATPESSGGSSGGS
Linker 1: 10aa flexible
(SEQ ID NO: 344)
GGGGSGGGGS
Linker 1: 10aa rigid
(SEQ ID NO: 345)
EAAAKEAAAK
Linker 1: 5aa rigid
(SEQ ID NO: 346)
EAAAK
10aa linker
(SEQ ID NO: 347)
SGGSGGSGGS
4aa linker
(SEQ ID NO: 348)
SGGS

### Supplementary Sequences 2|Split-DddAₜₒₓ-Cas9 Fusions Sequences

All split-DddAₜₒₓ-Cas9 have the general architecture of (from N- to C-terminus): bpNLS-DddAₜₒₓ half-32aa linker-dSpCas9 or SaKKH-Cas9(D10 Å)-10aa linker-UGI-10aa linker-UGI-4aa linker-bpNLS

G1333 DddAtox-N-
(SEQ ID NO: 349)
GSYALGPYQISAPQLPAYNGQTVGTFYYVNDAGGLESKVFSSGG
G1333 DddAtox-C
(SEQ ID NO: 350)
PTPYPNYANAGHVEGQSALFMRDNGISEGLVFHNNPEGTCGFCVNMTETLLPENAKMT
VVPPEGAIPVKRGATGETKVFTGNSNSPKSPTKGGC
G1397 DddAtox-N-
(SEQ ID NO: 351)
GSYALGPYQISAPQLPAYNGQTVGTFYYVNDAGGLESKVESSGGPTPYPNYANAGHVE
GQSALFMRDNGISEGLVFHNNPEGTCGFCVNMTETLLPENAKMTVVPPEG
G1397 DddAtox-C
(SEQ ID NO: 352)
AIPVKRGATGETKVFTGNSNSPKSPTKGGC
dSpCas9
(SEQ ID NO: 353)
DKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARENQT
TQKGQKNSRERMKRIEEGIKELGSQILKEHPVENTQLQNEKLYLYYLQNGRDMYVDQE
LDINRLSDYDVDAIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYD
ENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLY
LASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLYE
TRIDLSQLGGD
SaKKH-Cas9(D10A)
(SEQ ID NO: 354)
GKRNYILGLAIGITSVGYGIIDYETRDVIDAGVRLFKEANVENNEGRRSKRGARRLKRR
RRHRIQRVKKLLFDYNLLTDHSELSGINPYEARVKGLSQKLSEEEFSAALLHLAKRRGV
HNVNEVEEDTGNELSTKEQISRNSKALEEKYVAELQLERLKKDGEVRGSINRFKTSDYV
KEAKQLLKVQKAYHQLDQSFIDTYIDLLETRRTYYEGPGEGSPFGWKDIKEWYEMLM
GHCTYFPEELRSVKYAYNADLYNALNDLNNLVITRDENEKLEYYEKFQIIENVFKQKK
KPTLKQIAKEILVNEEDIKGYRVTSTGKPEFTNLKVYHDIKDITARKEIIENAELLDQIAKI
LTIYQSSEDIQEELTNLNSELTQEEIEQISNLKGYTGTHNLSLKAINLILDELWHTNDNQI
AIFNRLKLVPKKVDLSQQKEIPTTLVDDFILSPVVKRSFIQSIKVINAIIKKYGLPNDIIIEL
AREKNSKDAQKMINEMQKRNRQTNERIEEIIRTTGKENAKYLIEKIKLHDMQEGKCLYS
LEAIPLEDLLNNPFNYEVDHIIPRSVSFDNSFNNKVLVKQEENSKKGNRTPFQYLSSSDS
KISYETFKKHILNLAKGKGRISKTKKEYLLEERDINRFSVQKDFINRNLVDTRYATRGL
MNLLRSYFRVNNLDVKVKSINGGFTSFLRRKWKFKKERNKGYKHHAEDALIIANADFI
FKEWKKLDKAKKVMENQMFEEKQAESMPEIETEQEYKEIFITPHQIKHIKDFKDYKYSH
RVDKKPNRKLINDTLYSTRKDDKGNTLIVNNLNGLYDKDNDKLKKLINKSPEKLLMYH
HDPQTYQKLKLIMEQYGDEKNPLYKYYEETGNYLTKYSKKDNGPVIKKIKYYGNKLN
AHLDITDDYPNSRNKVVKLSLKPYRFDVYLDNGVYKFVTVKNLDVIKKENYYEVNSK
CYEEAKKLKKISNQAEFIASFYKNDLIKINGELYRVIGVNNDLLNRIEVNMIDITYREYL
ENMNDKRPPHIIKTIASKTQSIKKYSTDILGNLYEVKSKKHPQIIKKGGSPKKKRKVSSD
YKDHDGDYKDHDIDYKDDDDK
UGI
(SEQ ID NO: 355)
TNLSDIIEKETGKQLVIQESILMLPEEVEEVIGNKPESDILVHTAYDESTDENVMLLTSDA
PEYKPWALVIQDSNGENKIKML
bpNLS
(SEQ ID NO: 356)
KRTADGSEFEPKKKRKV
32 aa linker
(SEQ ID NO: 357)
SGGSSGGSSGSETPGTSESATPESSGGSSGGS
10 aa linker
(SEQ ID NO: 358)
SGGSGGSGGS
4 aa linker
(SEQ ID NO: 359)
SGGS

Supplementary Sequences 4 General DdCBE Architecture and mitoTALE Amino Acid Sequences

All right-side halves of DdCBEs have the general architecture of (from N- to C-terminus): COX8 Å MTS-3×FLAG-mitoTALE-2aa linker-DddAₜₒₓ half-4aa linker-1×-UGI-ATP5B 3′UTR

All left-side halves of DdCBEs have the general architecture of (from N- to C-terminus): SOD2 MTS-3×HA-mitoTALE-2aa linker-DddAₜₒₓ half-4aa linker-1×-UGI-SOD2 3′UTR

mitoTALE domains are annotated as: bold for N-terminal domain, underlined for RVD and bolded italics for C-terminal domain.

ND6-DdCBE: Left mitoTALE-G1397-DddAtox-N-1x-UGI (Note: Terminal NG RVD
recognizes a mismatched T instead of a G in the reference genome)
(SEQ ID NO: 360)
MALSRAVCGTSRQLAPVLGYLGSRQKHSLPDYPYDVPDYAGYPYDVPDYAGYPYDVP
DYAMDIADLRTLGYSQQQQEKIKPKVRSTVAQHHEALVGHGFTHAHIVALSQHPAAL
GTVAVKYQDMIAALPEATHEAIVGVGKQWSGARALEALLTVAGELRGPPLQLDTGQL
LKIAKRGGVTAVEAVHAWRNALTGAPLNLTPQQVVAIASNNGGKQALETVQRLLPVL
CQAHGLTPEQVVAIASNIGGKQALETVQALLPVLCQAHGLTPEQVVAIASHDGGKQAL
ETVQRLLPVLCQAHGLTPEQVVAIASHDGGKQALETVQRLLPVLCQAHGLTPEQVVAI
ASHDGGKQALETVQRLLPVLCQAHGLTPEQVVAIASHDGGKQALETVQRLLPVLCQA
HGLTPEQVVAIASHDGGKQALETVQRLLPVLCQAHGLTPEQVVAIASNIGGKQALETV
QALLPVLCQAHGLTPQQVVAIASNGGGKQALETVQRLLPVLCQAHGLTPQQVVAIASN
GGGRPALESIVAQLSRPDPALAALTNDHLVALACLGGRPALDAVKKGLGGSGSYALGP
YQISAPQLPAYNGQTVGTFYYVNDAGGLESKVFSSGGPTPYPNYANAGHVEGQSALFM
RDNGISEGLVFHNNPEGTCGFCVNMTETLLPENAKMTVVPPEGSGGSTNLSDIIEKETG
KQLVIQESILMLPEEVEEVIGNKPESDILVHTAYDESTDENVMLLTSDAPEYKPWALVIQ
DSNGENKIKML**
ND6-DdCBE: Right mitoTALE-G1397-DddAtox-N-1x-UGI (Note: Terminal NG RVD
recognizes a mismatched T instead of a G in the reference genome. The
NTD was also engineered to be permissive for A, T, C and G nucleotides
at the NO position)
(SEQ ID NO: 361)
MASVLTPLLLRGLTGSARRLPVPRAKIHSLDYKDHDGDYKDHDIDYKDDDDKMDIAD
LRTLGYSQQQQEKIKPKVRSTVAQHHEALVGHGFTHAHIVALSQHPAALGTVAVKYQ
DMIAALPEATHEAIVGVGKRGAGARALEALLTVAGELRGPPLQLDTGQLLKIAKRGGV
TAVEAVHAWRNALTGAPLNLTPQQVVAIASNNGGKQALETVQRLLPVLCQAHGLTPE
QVVAIASNIGGKQALETVQALLPVLCQAHGLTPQQVVAIASNGGGKQALETVQRLLPV
LCQAHGLTPQQVVAIASNNGGKQALETVQRLLPVLCQAHGLTPQQVVAIASNNGGKQ
ALETVQRLLPVLCQAHGLTPEQVVAIASHDGGKQALETVQRLLPVLCQAHGLTPQQVV
AIASNGGGKQALETVQRLLPVLCQAHGLTPEQVVAIASNIGGKQALETVQALLPVLCQ
AHGLTPQQVVAIASNGGGKQALETVQRLLPVLCQAHGLTPQQVVAIASNGGGKQALE
TVQRLLPVLCQAHGLTPQQVVAIASNGGGRPALESIVAQLSRPDPALAALTNDHLVAL
ACLGGRPALDAVKKGLGGSAIPVKRGATGETKVFTGNSNSPKSPTKGGCSGGSTNLSDI
IEKETGKQLVIQESILMLPEEVEEVIGNKPESDILVHTAYDESTDENVMLLTSDAPEYKP
WALVIQDSNGENKIKML**
ND1-DdCBE Right mitoTALE repeat
(SEQ ID NO: 362)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNNGGKQALETVQALLPVLCQAHGL
TPQQVVAIASNIGGKQALETVQRLLPVLCQAHGLTPQQVVAIASNNGGKQALETVQRL
LPVLCQAHGLTPEQVVAIASNGGGKQALETVQALLPVLCQAHGLTPEQVVAIASNGGG
KQALETVQALLPVLCQAHGLTPEQVVAIASNGGGKQALETVQALLPVLCQAHGLTPEQ
VVAIASNNGGKQALETVQRLLPVLCQAHGLTPEQVVAIASNIGGKQALETVQALLPVL
CQAHGLTPEQVVAIASNGGGKQALETVQRLLPVLCQAHGLTPEQVVAIASNNGGKQA
LETVQALLPVLCQAHGLTPEQVVAIASHDGGKQALETVQALLPVLCQAHGLTPEQVVA
IASNGGGKQALETVQALLPVLCQAHGLTPEQVVAIASHDGGKQALETVQRLLPVLCQA
HGLTPQQVVAIASNIGGKQALETVQRLLPVLCQAHGLTPEQVVAIASHDGGKQALETV
QALLPVLCQAHGLTPQQVVAIASHDGGKQALETVQRLLPVLCQAHGLTPQQVVAIASH
DGGKQALETVQRLLPVLCQAHGLTPQQVVAIASNGGGRPALESIVAQLSRPDPALAAL
TNDHLVALACLGGRPALDAVKKGLG
ND1-DdCBE Left mitoTALE repeat
(SEQ ID NO: 363)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNGGGKQALETVQRLLPVLCQAHGLTPQQVVAIASNIGGKQALETVQRL
LPVLCQAHGLTPEQVVAIASNNGGKQALETVQALLPVLCQAHGLTPEQVVAIASHDGG
KQALETVQALLPVLCQAHGLTPEQVVAIASHDGGKQALETVQALLPVLCQAHGLTPEQ
VVAIASNGGGKQALETVQRLLPVLCQAHGLTPEQVVAIASNIGGKQALETVQALLPVL
CQAHGLTPEQVVAIASNNGGKQALETVQRLLPVLCQAHGLTPEQVVAIASHDGGKQA
LETVQALLPVLCQAHGLTPEQVVAIASHDGGKQALETVQALLPVLCQAHGLTPEQVVA
IASNNGGKQALETVQALLPVLCQAHGLTPEQVVAIASNGGGKQALETVQRLLPVLCQA
HGLTPQQVVAIASNGGGKQALETVQRLLPVLCQAHGLTPQQVVAIASNGGGRPALESI
VAQLSRPDPALAALTNDHLVALACLGGRPALDAVKKGLG
ND2-DdCBE Right mitoTALE repeat
(SEQ ID NO: 364)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNIGGKQALETVQALLPVLCQAHGL
TPQQVVAIASNNGGKQALETVQRLLPVLCQAHGLTPQQVVAIASNIGGKQALETVQRL
LPVLCQAHGLTPEQVVAIASNIGGKQALETVQALLPVLCQAHGLTPEQVVAIASHDGG
KQALETVQALLPVLCQAHGLTPEQVVAIASNGGGKQALETVQALLPVLCQAHGLTPEQ
VVAIASNNGGKQALETVQRLLPVLCQAHGLTPEQVVAIASHDGGKQALETVQALLPVL
CQAHGLTPEQVVAIASNGGGKQALETVQRLLPVLCQAHGLTPEQVVAIASNIGGKQAL
ETVQALLPVLCQAHGLTPEQVVAIASNGGGKQALETVQALLPVLCQAHGLTPEQVVAI
ASNGGGKQALETVQALLPVLCQAHGLTPEQVVAIASNIGGKQALETVQRLLPVLCQAH
GLTPQQVVAIASNGGGKQALETVQRLLPVLCQAHGLTPQQVVAIASNGGGRPALESIV
AQLSRPDPALAALTNDHLVALACLGGRPALDAVKKGLG
ND2-DdCBE Left mitoTALE repeat
(SEQ ID NO: 365)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNGGGKQALETVQRLLPVLCQAHGLTPQQVVAIASNGGGKQALETVQR
LLPVLCQAHGLTPEQVVAIASNIGGKQALETVQALLPVLCQAHGLTPEQVVAIASNNG
GKQALETVQALLPVLCQAHGLTPEQVVAIASHDGGKQALETVQALLPVLCQAHGLTPE
QVVAIASNIGGKQALETVQRLLPVLCQAHGLTPEQVVAIASNGGGKQALETVQALLPV
LCQAHGLTPEQVVAIASNIGGKQALETVQRLLPVLCQAHGLTPEQVVAIASHDGGKQA
LETVQALLPVLCQAHGLTPEQVVAIASNGGGKQALETVQALLPVLCQAHGLTPEQVVA
IASHDGGKQALETVQALLPVLCQAHGLTPEQVVAIASHDGGKQALETVQRLLPVLCQA
HGLTPQQVVAIASNGGGKQALETVQRLLPVLCQAHGLTPEQVVAIASHDGGKQALETV
QALLPVLCQAHGLTPEQVVAIASNIGGKQALETVQALLPVLCQAHGLTPEQVVAIASNI
GGKQALETVQALLPVLCQAHGLTPQQVVAIASNGGGRPALESIVAQLSRPDPALAALT
NDHLVALACLGGRPALDAVKKGLG
ND4-DdCBE Right mitoTALE repeat
(SEQ ID NO: 366)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASHDGGKQALETVQRLLPVLCQAHGLTPQQVVAIASNGGGKQALETVQR
LLPVLCQAHGLTPEQVVAIASNNGGKQALETVQALLPVLCQAHGLTPEQVVAIASNGG
GKQALETVQALLPVLCQAHGLTPEQVVAIASNIGGKQALETVQALLPVLCQAHGLTPE
QVVAIASNIGGKQALETVQRLLPVLCQAHGLTPEQVVAIASNNGGKQALETVQALLPV
LCQAHGLTPEQVVAIASNGGGKQALETVQRLLPVLCQAHGLTPEQVVAIASNIGGKQA
LETVQRLLPVLCQAHGLTPEQVVAIASNNGGKQALETVQALLPVLCQAHGLTPEQVVA
IASNNGGKQALETVQALLPVLCQAHGLTPEQVVAIASNIGGKQALETVQALLPVLCQA
HGLTPEQVVAIASNNGGKQALETVQRLLPVLCQAHGLTPQQVVAIASNIGGKQALETV
QRLLPVLCQAHGLTPQQVVAIASNNGGKQALETVQRLLPVLCQAHGLTPQQVVAIASN
GGGRPALESIVAQLSRPDPALAALTNDHLVALACLGGRPALDAVKKGLG
ND4-DdCBE Left mitoTALE repeat
(SEQ ID NO: 367)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNNGGKQALETVQALLPVLCQAHGL
TPQQVVAIASHDGGKQALETVQRLLPVLCQAHGLTPQQVVAIASNGGGKQALETVQR
LLPVLCQAHGLTPEQVVAIASNIGGKQALETVQALLPVLCQAHGLTPEQVVAIASNNG
GKQALETVQALLPVLCQAHGLTPEQVVAIASNGGGKQALETVQALLPVLCQAHGLTPE
QVVAIASNIGGKQALETVQRLLPVLCQAHGLTPEQVVAIASNIGGKQALETVQALLPVL
CQAHGLTPEQVVAIASHDGGKQALETVQRLLPVLCQAHGLTPEQVVAIASHDGGKQA
LETVQALLPVLCQAHGLTPEQVVAIASNIGGKQALETVQALLPVLCQAHGLTPEQVVAI
ASHDGGKQALETVQALLPVLCQAHGLTPEQVVAIASNNGGKQALETVQRLLPVLCQA
HGLTPQQVVAIASNGGGKQALETVQRLLPVLCQAHGLTPEQVVAIASNGGGKQALETV
QALLPVLCQAHGLTPEQVVAIASHDGGKQALETVQALLPVLCQAHGLTPQQVVAIASN
GGGRPALESIVAQLSRPDPALAALTNDHLVALACLGGRPALDAVKKGLG
ND5.1-DdCBE Right mitoTALE repeat
(SEQ ID NO: 368)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASHDGGKQALETVQRLLPVLCQAHGL
TPEQVVAIASNGGGKQALETVQRLLPVLCQAHGLTPQQVVAIASNGGGKQALETVQRL
LPVLCQAHGLTPEQVVAIASNGGGKQALETVQALLPVLCQAHGLTPQQVVAIASNNGG
KQALETVQRLLPVLCQAHGLTPQQVVAIASNNGGKQALETVQRLLPVLCQAHGLTPEQ
VVAIASNIGGKQALETVQRLLPVLCQAHGLTPQQVVAIASNNGGKQALETVQRLLPVL
CQAHGLTPQQVVAIASNGGGKQALETVQRLLPVLCQAHGLTPEQVVAIASNIGGKQAL
ETVQRLLPVLCQAHGLTPEQVVAIASNNGGKQALETVQRLLPVLCQAHGLTPQQVVAI
ASNIGGKQALETVQRLLPVLCQAHGLTPQQVVAIASNIGGRPALESIVAQLSRPDPALA
ALTNDHLVALACLGGRPALDAVKKGLG
ND5.1-DdCBE Left mitoTALE repeat
(SEQ ID NO: 369)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNIGGKQALETVQALLPVLCQAHGL
TPQQVVAIASNNGGKQALETVQRLLPVLCQAHGLTPQQVVAIASHDGGKQALETVQR
LLPVLCQAHGLTPEQVVAIASNIGGKQALETVQALLPVLCQAHGLTPEQVVAIASNGG
GKQALETVQALLPVLCQAHGLTPEQVVAIASNGGGKQALETVQALLPVLCQAHGLTPE
QVVAIASNIGGKQALETVQRLLPVLCQAHGLTPEQVVAIASNNGGKQALETVQALLPV
LCQAHGLTPEQVVAIASHDGGKQALETVQRLLPVLCQAHGLTPEQVVAIASNIGGKQA
LETVQALLPVLCQAHGLTPEQVVAIASNNGGKQALETVQALLPVLCQAHGLTPEQVVA
IASNNGGKQALETVQALLPVLCQAHGLTPEQVVAIASNIGGKQALETVQRLLPVLCQA
HGLTPQQVVAIASNIGGKQALETVQRLLPVLCQAHGLTPQQVVAIASNGGGRPALESIV
AQLSRPDPALAALTNDHLVALACLGGRPALDAVKKGLG
ND5.2-DdCBE Right mitoTALE repeat (Note: Terminal NG RVD recognizes a
mismatched T instead of a G in the reference genome)
(SEQ ID NO: 370)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASNNGGKQALETVQRLLPVLCQAHGL
TPEQVVAIASHDGGKQALETVQRLLPVLCQAHGLTPQQVVAIASNGGGKQALETVQRL
LPVLCQAHGLTPEQVVAIASNIGGKQALETVQALLPVLCQAHGLTPQQVVAIASNNGG
KQALETVQRLLPVLCQAHGLTPQQVVAIASNNGGKQALETVQRLLPVLCQAHGLTPEQ
VVAIASHDGGKQALETVQRLLPVLCQAHGLTPQQVVAIASNGGGKQALETVQRLLPVL
CQAHGLTPQQVVAIASNNGGKQALETVQRLLPVLCQAHGLTPEQVVAIASHDGGKQA
LETVQRLLPVLCQAHGLTPEQVVAIASHDGGKQALETVQRLLPVLCQAHGLTPEQVVA
IASNIGGKQALETVQALLPVLCQAHGLTPEQVVAIASNIGGKQALETVQALLPVLCQAH
GLTPQQVVAIASNGGGKQALETVQRLLPVLCQAHGLTPQQVVAIASNNGGKQALETV
QRLLPVLCQAHGLTPQQVVAIASNGGGRPALESIVAQLSRPDPALAALTNDHLVALAC
LGGRPALDAVKKGLG
ND5.2-DdCBE Left mitoTALE repeat
(SEQ ID NO: 371)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNIGGKQALETVQRLLPVLCQAHGLTPQQVVAIASNIGGKQALETVQRLL
PVLCQAHGLTPEQVVAIASNIGGKQALETVQALLPVLCQAHGLTPEQVVAIASNIGGKQ
ALETVQALLPVLCQAHGLTPEQVVAIASHDGGKQALETVQALLPVLCQAHGLTPEQVV
AIASHDGGKQALETVQRLLPVLCQAHGLTPEQVVAIASNIGGKQALETVQALLPVLCQ
AHGLTPEQVVAIASNGGGKQALETVQRLLPVLCQAHGLTPEQVVAIASNIGGKQALET
VQALLPVLCQAHGLTPEQVVAIASHDGGKQALETVQALLPVLCQAHGLTPEQVVAIAS
HDGGKQALETVQALLPVLCQAHGLTPEQVVAIASNGGGKQALETVQRLLPVLCQAHG
LTPQQVVAIASHDGGKQALETVQRLLPVLCQAHGLTPQQVVAIASNGGGRPALESIVA
QLSRPDPALAALTNDHLVALACLGGRPALDAVKKGLG
ND5.3-DdCBE Right mitoTALE repeat
(SEQ ID NO: 372)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASNIGGKQALETVQRLLPVLCQAHGL
TPEQVVAIASNIGGKQALETVQRLLPVLCQAHGLTPQQVVAIASNNGGKQALETVQRL
LPVLCQAHGLTPEQVVAIASNIGGKQALETVQALLPVLCQAHGLTPQQVVAIASNGGG
KQALETVQRLLPVLCQAHGLTPQQVVAIASNGGGKQALETVQRLLPVLCQAHGLTPEQ
VVAIASNIGGKQALETVQRLLPVLCQAHGLTPQQVVAIASNNGGKQALETVQRLLPVL
CQAHGLTPQQVVAIASNGGGKQALETVQRLLPVLCQAHGLTPEQVVAIASNIGGKQAL
ETVQRLLPVLCQAHGLTPEQVVAIASNGGGKQALETVQRLLPVLCQAHGLTPQQVVAI
ASNNGGKQALETVQRLLPVLCQAHGLTPQQVVAIASNNGGKQALETVQRLLPVLCQA
HGLTPQQVVAIASNGGGRPALESIVAQLSRPDPALAALTNDHLVALACLGGRPALDAV
KKGLG
ND5.3-DdCBE Left mitoTALE repeat
(SEQ ID NO: 373)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASHDGGKQALETVQALLPVLCQAHGL
TPQQVVAIASNIGGKQALETVQRLLPVLCQAHGLTPQQVVAIASNNGGKQALETVQRL
LPVLCQAHGLTPEQVVAIASNGGGKQALETVQALLPVLCQAHGLTPEQVVAIASNGGG
KQALETVQALLPVLCQAHGLTPEQVVAIASHDGGKQALETVQALLPVLCQAHGLTPEQ
VVAIASNGGGKQALETVQRLLPVLCQAHGLTPEQVVAIASNGGGKQALETVQALLPVL
CQAHGLTPEQVVAIASHDGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIA
SNIGGKQALETVQALLPVLCQAHGLTPEQVVAIASNGGGKQALETVQRLLPVLCQAHG
LTPQQVVAIASNIGGKQALETVQRLLPVLCQAHGLTPQQVVAIASNGGGRPALESIVAQ
LSRPDPALAALTNDHLVALACLGGRPALDAVKKGLG
ATP8-DdCBE Right mitoTALE repeat (Note: Terminal NG RVD recognizes a
mismatched T instead of a C in the reference genome)
(SEQ ID NO: 374)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPQQVVAIASNIGGKQALETVQRLLPVLCQAHGL
TPEQVVAIASNGGGKQALETVQRLLPVLCQAHGLTPQQVVAIASNNGGKQALETVQRL
LPVLCQAHGLTPEQVVAIASNNGGKQALETVQALLPVLCQAHGLTPQQVVAIASNNGG
KQALETVQRLLPVLCQAHGLTPQQVVAIASHDGGKQALETVQRLLPVLCQAHGLTPEQ
VVAIASNGGGKQALETVQRLLPVLCQAHGLTPQQVVAIASNGGGKQALETVQRLLPVL
CQAHGLTPQQVVAIASNGGGKQALETVQRLLPVLCQAHGLTPEQVVAIASNNGGKQA
LETVQRLLPVLCQAHGLTPEQVVAIASNNGGKQALETVQRLLPVLCQAHGLTPQQVVA
IASNGGGRPALESIVAQLSRPDPALAALTNDHLVALACLGGRPALDAVKKGLG
ATP8-DdCBE Left mitoTALE repeat
(SEQ ID NO: 375)
DIADLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNIGGKQALETVQALLPVLCQAHGL
TPQQVVAIASNGGGKQALETVQRLLPVLCQAHGLTPQQVVAIASNGGGKQALETVQR
LLPVLCQAHGLTPEQVVAIASNIGGKQALETVQALLPVLCQAHGLTPEQVVAIASNIGG
KQALETVQALLPVLCQAHGLTPEQVVAIASNIGGKQALETVQALLPVLCQAHGLTPEQ
VVAIASHDGGKQALETVQRLLPVLCQAHGLTPEQVVAIASNIGGKQALETVQALLPVL
CQAHGLTPEQVVAIASHDGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIA
SNIGGKQALETVQALLPVLCQAHGLTPEQVVAIASHDGGKQALETVQRLLPVLCQAHG
LTPQQVVAIASNGGGKQALETVQRLLPVLCQAHGLTPEQVVAIASNIGGKQALETVQA
LLPVLCQAHGLTPQQVVAIASNGGGRPALESIVAQLSRPDPALAALTNDHLVALACLG
GRPALDAVKKGLG

### Example 2: New DddA Mutants with Improved Editing Activity at TC Context

As noted in Example 1, DdCBE editing activity at selected sites, including MT-ATP8 and MT-ND5.2, remained low (<10%) across all possible G1333 and G1397 split orientations. Phage-assisted non-continuous and continuous evolution (PANCE and PACE) was applied to evolve split-DddA (Thuronyi, B. W. et al. Nat Biotechnol 37, 1070-1079(2019)). (FIG. 59), resulting in variants that contained mutations in the N-terminal and C-terminal halves of split DddA (FIGS. 60A-60D). Exemplary variant sequences are shown:

| | | SEQ ID |
|---|---|---|
| Mutation(s) | Sequence | NO: |
| DddA (residues | GSYALGPYQISAPQLPAYNGQTVGTFYYVNDAGGLESKVFSSGGPTP | 338 |
| 1290-1427) | YPNYANAGHVEGQSALFMRDNGISEGLVFHNNPEGTCGFCVNMTET | |
| | LLPENAKMTVVPPEGAIPVKRGATGETKVFTGNSNSPKSPTKGGC | |
| T1380I | GSYALGPYQISAPQLPAYNGQTVGTFYYVNDAGGLESKVFSSGGPTP | 377 |
| | YPNYANAGHVEGQSALFMRDNGISEGLVFHNNPEGTCGFCVNMIETL | |
| | LPENAKMTVVPPEGAIPVKRGATGETKVFTGNSNSPKSPTKGGC | |
| T1314A/T1380I | GSYALGPYQISAPQLPAYNGQTVGAFYYVNDAGGLESKVFSSGGPTP | 378 |
| | YPNYANAGHVEGQSALFMRDNGISEGLVFHNNPEGTCGFCVNMIETL | |
| | LPENAKMTVVPPEGAIPVKRGATGETKVFTGNSNSPKSPTKGGC | |
| Q1310R/S1330I/ | GSYALGPYQISAPQLPAYNGRTVGTFYYVNDAGGLESKVFISGGPTP | 379 |
| T1380I | YPNYANAGHVEGQSALFMRDNGISEGLVFHNNPEGTCGFCVNMIETL | |
| | LPENAKMTVVPPEGAIPVKRGATGETKVFTGNSNSPKSPTKGGC | |
| T1380I/T1413I | GSYALGPYQISAPQLPAYNGQTVGTFYYVNDAGGLESKVFSSGGPTP | 380 |
| | YPNYANAGHVEGQSALFMRDNGISEGLVFHNNPEGTCGFCVNMIETL | |
| | LPENAKMTVVPPEGAIPVKRGATGETKVFIGNSNSPKSPTKGGC | |
| T1314A/T1380I/ | GSYALGPYQISAPQLPAYNGQTVGAFYYVNDAGGLESKVFSSGGPTP | 381 |
| E1396K | YPNYANAGHVEGQSALFMRDNGISEGLVFHNNPEGTCGFCVNMIETL | |
| | LPENAKMTVVPPKGAIPVKRGATGETKVFTGNSNSPKSPTKGGC | |

These DddA mutations were cloned into the DdCBE architecture (MTS-right TALE-G1397 split DddA-(N/C)-UGI) and plasmid transfected into human HEK293T cells (FIG. 60A). At sites that were weakly editing by the wildtype DdCBE, editor variant T1380I improved editing efficiencies 1.2-2.3 fold (FIG. 60B), and variants Q1310R/S1330I/T1380I and T1380I/T1413I improved editing efficiencies by 4.8-4.9-fold up to 40%. (FIG. 60C). The indels associated with the highly active variants remained below the 0.1% detection limit (FIG. 60D). Using directed protein evolution, split G1397-DddA variants that show greatly improved editing efficiencies over the wildtype DddA were successfully evolved.

### Example 3. Lentiviral Delivery of DdCBEs into Mouse Cells

The ability of DdCBEs to install disease-causing mutations in animal models will accelerate the study of disease etiology and facilitate preclinical testing of drug candidates. In particular, mutations in mitochondrial genes encoding for Complex I subunits are increasingly studied for their role in cancer (Gopal, R. K. et al. Cancer Cell 34, 242-255.e245(2018)). DdCBEs were designed that target various Complex I genes in the mouse mtDNA to install a pathogenic missense mutation. The right and left halves of each DdCBE were cloned separately into a lentiviral vector and co-transfected into mouse embryonic fibroblasts (FIG. 61). Sanger sequencing of cells that express both DdCBEs halves showed efficient DdCBE editing at the target mtDNA site (FIG. 62). Compared to untreated cells, cells containing these pathogenic mutations had lower oxygen consumption rates and higher extracellular acidification rates, indicating a preference for glycolysis over oxidative phosphorylation (FIG. 63). These results collectively indicate that DdCBEs is compatible with existing viral delivery platforms and can be applied to generate relevant disease cell lines, and potentially animal models.

### Example 4. Alternative DdCBE Architectures

The original DdCBE design, described in Example 1, contains a Left-TALE that targets the top coding DNA strand and a Right-TALE that targets the bottom coding DNA strand (FIG. 64A). TALEs that bind in this orientation brings the two inactive DddA halves in close proximity for reassembly. It was observed that mtDNA editing efficiencies and editing window are partly dependent on the length of the spacing region and the position of the target C within the spacing region. These parameters can be optimized through testing of different TALE sequences. This effort, however, has been hampered by strict guidelines governing the design of high affinity-binding TALEs (Rogers, J. M. et al. Nat Commun 6, 7440 (2015)).. To expand the options for TALE design three other alternative DdCBE architectures were generated. In the “Opposite” design, the Left-TALE binds to the bottom DNA strand and the Right-TALE binds to the top DNA strand (FIG. 64B). For DdCBEs containing monomers that bind to the same DNA strand, the TALEs can target either the top strand or bottom strand (FIG. 64B). To maintain editing activity in these alternative architectures, the MTS is fused to the N-terminus and the UGI must be distal to the MTS for localization of the editor into the mitochondria.

In general, the editing efficiencies observed for the alternative DdCBE architectures were generally lower than the original DdCBE design (FIG. 65). In particular, the Opposite architecture abrogated editing at target cytosines that had been most efficiently edited by the Original design (up to 35%). These results indicate that while these alternative DdCBE architectures remain active, they should only be used when there are no available TALE binding sites that correspond to the Original architecture. An exemplary protein sequence is shown below:

MTS-split DddA half G1397-N-ND1 Right TALE
repeat-UGI
(SEQ ID NO: 420)
SVLTPLLLRGLTGSARRLPVPRAKIHSLMGSYALGPYQISAPQLPAYNG
QTVGTFYYVNDAGGLESKVFSSGGPTPYPNYANAGHVEGQSALFMRDNG
ISEGLVFHNNPEGTCGFCVNMTETLLPENAKMTVVPPEGGSDIADLRTL
GYSQQQQEKIKPKVRSTVAQHHEALVGHGFTHAHIVALSQHPAALGTVA
VKYQDMIAALPEATHEAIVGVGKQWSGARALEALLTVAGELRGPPLQLD
TGQLLKIAKRGGVTAVEAVHAWRNALTGAPLNLTPEQVVAIASHDGGKQ
ALETVQALLPVLCQAHGLTPQQVVAIASNIGGKQALETVQRLLPVLCQA
HGLTPQQVVAIASNNGGKQALETVQRLLPVLCQAHGLTPEQVVAIASNN
GGKQALETVQALLPVLCQAHGLTPEQVVAIASNNGGKQALETVQALLPV
LCQAHGLTPEQVVAIASNGGGKQALETVQALLPVLCQAHGLTPEQVVAI
ASNNGGKQALETVQRLLPVLCQAHGLTPEQVVAIASNIGGKQALETVQA
LLPVLCQAHGLTPEQVVAIASNNGGKQALETVQRLLPVLCQAHGLTPEQ
VVAIASHDGGKQALETVQALLPVLCQAHGLTPEQVVAIASNIGGKQALE
TVQALLPVLCQAHGLTPQQVVAIASNGGGRPALESIVAQLSRPDPALAA
LTNDHLVALACLGGRPALDAVKKGLGSGGSTNLSDIIEKETGKQLVIQE
SILMLPEEVEEVIGNKPESDILVHTAYDESTDENVMLLTSDAPEYKPWA
LVIQDSNGENKIKML

### Example 5. Mitochondrial Base Editing with ZF-BE

A process for ZF-BE base editing was designed as an alternative to TALE-DdCBE described in the Examples above. As ZFs (0.5kb) are smaller than TALE (2kb), it was believed that ZF-BEs would allow for better expression and lower immunogenicity, as well as single AAV base editing (2×1.2 kb). However, to date, no published pairs of mitoZFs exist which target the wild-type genome sequence. As a result, ZFs were designed to be used in combination previously reported mitoZFs R13-1 and R8 (Gammage et al. EMBO Mol Med. 2014 Apr;6(4):458-66.)

### ZF-BEs Design

ZF-BEs were designed to comprise the following components:
Mitochondrial Targeting Sequence (MTS) for import of a cytosolic-located protein into the mitochondria. Localisation of the ZF-BE in the mitochondria is critical for its ability to edit mitochondrial DNA.
FLAG tag which serves two roles—firstly as an affinity tag allowing both expression levels of the ZF-BE to be determined by Western Blot using an anti-FLAG antibody, and intracellular localization to be imaged by antibody staining of transfected cells by microscopy. Secondly, as a disordered sequence following the MTS which improves mitochondrial import via the MTS, facilitating better loading and protein unfolding through mitochondrial import channel.
Nuclear Export Sequence (NES) for export of a nuclear-located protein into the cytosol, where it can then be transported into the mitochondria via its MTS. Since ZFs inherently encode a Nuclear Localisation Sequence (NLS), this is important to counteract their natural tendency to localize in the nucleus which is problematic for editing mitochondrial DNA. Utilising two separate NES boosts the cytoplasmic localization of ZF-BEs by further reducing their nuclear localization, enabling stronger uptake of ZF-BEs into the mitochondria for BE to occur.
Zinc Finger (ZF) as a DNA-binding domain able to specify the target sequence for the ZF-BE to bind to within mitochondrial DNA and target the split DddA deaminase towards site-specific base editing.
Split DddA deaminase, capable of performing deamination on dsDNA when reconstituted into a full-length DddA at the target site within mitochondrial DNA when both halves of the DddA deaminase are colocalized.
Uracil glycosylase inhibitor (UGI) for binding to mitochondrial uracil DNA glycosylase (UDG) and inhibiting the activity of UDG to excise modified uracil bases from edited DNA.
P2 Å peptide enabling coexpression of two independent polypeptides from a single gene-encoding sequence due to the inability of the mammalian ribosome to form complete polypeptide bond formation during translation. This is used to enable coexpression of an additional copy of UGI from the same gene-encoding sequence which can independently localize into mitochondria via its own MTS, increasing the level of mitochondrial UDG inhibition.

### Optimisation of ZF-BE Architecture

ZFs were designed to target two sites in the mitochondrial genome: ATP8 (referred to as R8) and ND5.1 (referred to as R13). ZFs were designed using a modular assembly approach, using a modified Zif268 scaffold. At each site ZFs were designed to form one half of a pair alongside R8 or R13-1. The resulting set of ZFs bound to sequences and created an editing window between the ZF pair, ranging from 4-18 bp. Binding motifs are shown in FIG. 66. Results from initial experiments (v1) are shown in FIGS. 67-70D. Based on this data, designed ZFs 5×ZnF-4-R8 and 5×ZnF-10-R8 (site R8), and 5×ZnF-9-R13 and 5×ZnF-12-R13 (site R13) were deemed to be the best performing.

The architecture of the lead v1 ZF-BE candidates were varied with regards to MTS, NES and linker length (v2) (FIGS. 71A-71B). Results showed that v1 MTS and NES outperformed v2 MTS1/2 and NES1/2 (FIGS. 72-75B). The optimal linker length varied between different sites, suggesting that varying linker length may be a route to restricting the editing window. Link13 was chosen for further experimentation as it returned the best results across both sites. Results also showed that varying the ZF binding site, and in turn the editing window size, appeared to be an effective route for changing targeted nucleotide edits.

The ZF-BE architecture was further optimized by retaining the Link13 flexible linker and the N-terminal MTS and NES from v2 experiments. V3 systematically varied the MTS in the presence or absence of an affinity tag sequence (FLAG or HA) immediately following the MTS (FIG. 76). Results showed that the Minczuk (Mzk) MTS in the presence of a FLAG tag was the best performing architecture (FIGS. 77A-77D) and these improvements were used in further experimentation.

The next round of optimization tested additional NES sequences (x3), different UGI homologs (x2) and ZF scaffold mutations (FIG. 78, v4). Results showed that adding an extra NES to the c-terminus (“NESX”) did not improve editing efficiencies. However, adding an extra internal NES (“IntNES2”) downstream of the existing NES improved editing efficiencies (FIGS. 79A-79D) and was carried forward in further experimentation. Results of editing with alternative UGI homologs did not show any improvement to ZF base editing (FIGS. 80A-80D). ZFs naturally localize in the nucleus and the ZF sequence is necessary and sufficient for nuclear localization. However, nuclear localization is not dependent or tertiary structure and mutation of conserved His/Cys residues which coordinate Zn²⁺ does not perturb nuclear localization. As a result, mutation of positively charged residues can reduce nuclear localization. FIGS. 81A-81C show ZF scaffolding with non-conserved positively charged residues in bold. These residues were targeted for mutations (FIG. 81C) to ablate the positive charge and reduce the overall NLS quality of the ZF. Results of ZF scaffold mutations showed that the N and combined DN set of mutations did not improve editing efficiency. However, the D mutation set improved editing efficiency and these mutations were carried forward for further experimentation (FIGS. 82A-82D).

The D mutations were combined with previous architecture improvements (FIG. 83, v5). Results confirmed that D mutations are beneficial and that combining these with the additional internal NES could improve editing efficiency further (D2=D+IntNES2; D3=D+IntNES3) (FIGS. 84A-84D). V5 experiments showed that moving the UGI to the N-terminus did not improve results. However, increasing the amount of UGI delivered to the mitochdria, either by appending 2× UGI or by following the editor with an extra copy of UGI expressed in trans and targeted to mitochondria via its own MTS, led to an improvement in editing efficiency (FIGS. 85A-85D).

The final round of optimization included an additional NES, improvement of the ZF scaffold sequence, and coexpression of a separate mitochondrially-targeted UGI (FIG. 86, v6). Inclusion of mutations T1380I, E1396K and T1413I into the split DddA deaminase halves were also tested (FIG. 86, v6M). Sequences for architectures shown FIG. 86 are described below. Results of editing efficiency are shown in FIG. 87B.

| | | |
|---|---|---|
| R8 v6 | MLGFVGRVAAAPASGALRRLTPSASLPP | MTS |
| | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 394) |
| | LEELELDAAMAERPFQCDICMRNFSTSG | FLAG tag |
| | SLSRHIRTHTGEKPFQCDICMRNFSQSGS | DYKDDDDK (SEQ ID NO: 395) |
| | LTRHIRTHTGSEKPFQCDICMRNFSRSDA | NES |
| | LSQHIRTHTGEKPFQCDICMRNFSRNDN | VDEMTKKFGTLTIHDTEK (SEQ ID NO: 396) |
| | RITHIRTHTGEKPFQCDICMRNFSRSDHL | Linker |
| | TQHTKIHLRGSGGGGSGGSGGSGSYAL | GS |
| | GPYQISAPQLPAYNGQTVGTFYYVNDA | NES2 |
| | GGLESKVFSSGGPTPYPNYANAGHVEG | LQKKLEELELD (SEQ ID NO: 397) |
| | QSALFMRDNGISEGLVFHNNPEGTCGFC | Linker |
| | VNMTETLLPENAKMTVVPPEGSGGSTN | AA |
| | LSDIIEKETGKQLVIQESILMLPEEVEEVI | ZF (R8) |
| | GNKPESDILVHTAYDESTDENVMLLTSD | MAERPFQCDICMRNFSTSGSLSRHIRTHTGEKPF |
| | APEYKPWALVIQDSNGENKIKMLGSGA | QCDICMRNFSQSGSLTRHIRTHTGSEKPFQCDIC |
| | TNFSLLKQAGDVEENPGPMASVLTPLLL | MRNFSRSDALSQHIRTHTGEKPFQCDICMRNFS |
| | RGLTGSARRLPVPRAKIHSLGSTNLSDII | RNDNRITHIRTHTGEKPFQCDICMRNFSRSDHL |
| | EKETGKQLVIQESILMLPEEVEEVIGNKP | TQHTKIHLR (SEQ ID NO: 398) |
| | ESDILVHTAYDESTDENVMLLTSDAPEY | Linker |
| | KPWALVIQDSNGENKIKML (SEQ ID NO: | GSGGGGSGGSGGS (SEQ ID NO: 399) |
| | 382) | Split DddA (DddA-G1397N) |
| | | GSYALGPYQISAPQLPAYNGQTVGTFYYVNDA |
| | | GGLESKVFSSGGPTPYPNYANAGHVEGQSALF |
| | | MRDNGISEGLVFHNNPEGTCGFCVNMTETLLP |
| | | ENAKMTVVPPEG (SEQ ID NO: 284) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| | | Linker |
| | | GSG |
| | | P2A Peptide |
| | | ATNFSLLKQAGDVEENPGP (SEQ ID NO: 400) |
| | | MTS |
| | | MASVLTPLLLRGLTGSARRLPVPRAKIHSL |
| | | (SEQ ID NO: 401) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| 5xZnF-4- | MLGFVGRVAAAPASGALRRLTPSASLPP | MTS |
| R8 v6 | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 394) |
| | LEELELDAAMAERPFQCDICMRNFSQAS | FLAG tag |
| | NLISHIRTHTGEKPFQCDICMRNFSTSHS | DYKDDDDK (SEQ ID NO: 395) |
| | LTEHIRTHTGSEKPFQCDICMRNFSERSH | NES |
| | LREHIRTHTGEKPFQCDICMRNFSQSGN | VDEMTKKFGTLTIHDTEK (SEQ ID NO: 396) |
| | LTEHIRTHTGEKPFQCDICMRNFSSKKA | Linker |
| | LTEHTKIHLRGSGGGGSGGSGGSAIPVK | GS |
| | RGATGETKVFTGNSNSPKSPTKGGCSGG | NES2 |
| | STNLSDIIEKETGKQLVIQESILMLPEEVE | LQKKLEELELD (SEQ ID NO: 397) |
| | EVIGNKPESDILVHTAYDESTDENVMLL | Linker |
| | TSDAPEYKPWALVIQDSNGENKIKMLGS | AA |
| | GATNFSLLKQAGDVEENPGPMASVLTP | ZF (5xZnF-4-R8) |
| | LLLRGLTGSARRLPVPRAKIHSLGSTNLS | MAERPFQCDICMRNFSQASNLISHIRTHTGEKPF |
| | DIIEKETGKQLVIQESILMLPEEVEEVIGN | QCDICMRNFSTSHSLTEHIRTHTGSEKPFQCDIC |
| | KPESDILVHTAYDESTDENVMLLTSDAP | MRNFSERSHLREHIRTHTGEKPFQCDICMRNFS |
| | EYKPWALVIQDSNGENKIKML (SEQ ID | QSGNLTEHIRTHTGEKPFQCDICMRNESSKKAL |
| | NO: 383) | TEHTKIHLR (SEQ ID NO: 402) |
| | | Linker |
| | | GSGGGGSGGSGGS (SEQ ID NO: 399) |
| | | Split DddA (DddA-G1397C) |
| | | AIPVKRGATGETKVFTGNSNSPKSPTKGGC |
| | | (SEQ ID NO: 286) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| | | Linker |
| | | GSG |
| | | P2A Peptide |
| | | ATNFSLLKQAGDVEENPGP (SEQ ID NO: 400) |
| | | MTS |
| | | MASVLTPLLLRGLTGSARRLPVPRAKIHSL |
| | | (SEQ ID NO: 401) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| 5xZnF-10- | MLGFVGRVAAAPASGALRRLTPSASLPP | MTS |
| R8 v6 | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 394) |
| | LEELELDAAMAERPFQCDICMRNFSQAS | FLAG tag |
| | NLISHIRTHTGEKPFQCDICMRNFSQRAN | DYKDDDDK (SEQ ID NO: 395) |
| | LRAHIRTHTGSEKPFQCDICMRNFSQAS | NES |
| | NLISHIRTHTGEKPFQCDICMRNFSTSHS | VDEMTKKFGTLTIHDTEK (SEQ ID NO: 396) |
| | LTEHIRTHTGEKPFQCDICMRNFSERSHL | Linker |
| | REHTKIHLRGSGGGGSGGSGGSAIPVKR | GS |
| | GATGETKVFTGNSNSPKSPTKGGCSGGS | NES2 |
| | TNLSDIIEKETGKQLVIQESILMLPEEVEE | LQKKLEELELD (SEQ ID NO: 397) |
| | VIGNKPESDILVHTAYDESTDENVMLLT | Linker |
| | SDAPEYKPWALVIQDSNGENKIKMLGS | AA |
| | GATNFSLLKQAGDVEENPGPMASVLTP | ZF (5xZnF-10-R8) |
| | LLLRGLTGSARRLPVPRAKIHSLGSTNLS | MAERPFQCDICMRNFSQASNLISHIRTHTGEKPF |
| | DIIEKETGKQLVIQESILMLPEEVEEVIGN | QCDICMRNFSQRANLRAHIRTHTGSEKPFQCDI |
| | KPESDILVHTAYDESTDENVMLLTSDAP | CMRNFSQASNLISHIRTHTGEKPFQCDICMRNFS |
| | EYKPWALVIQDSNGENKIKML (SEQ ID | TSHSLTEHIRTHTGEKPFQCDICMRNFSERSHLR |
| | NO: 384) | EHTKIHLR (SEQ ID NO: 403) |
| | | Linker |
| | | GSGGGGSGGSGGS (SEQ ID NO: 399) |
| | | Split DddA (DddA-G1397C) |
| | | AIPVKRGATGETKVFTGNSNSPKSPTKGGC |
| | | (SEQ ID NO: 286) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| | | Linker |
| | | GSG |
| | | P2A Peptide |
| | | ATNFSLLKQAGDVEENPGP (SEQ ID NO: 400) |
| | | MTS |
| | | MASVLTPLLLRGLTGSARRLPVPRAKIHSL |
| | | (SEQ ID NO: 401) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| R13-1 v6 | MLGFVGRVAAAPASGALRRLTPSASLPP | MTS |
| | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 394) |
| | LEELELDAAMAERPFQCDICMRNFSRSD | FLAG tag |
| | NLSTHIRTHTGEKPFQCDICMRNFSDRS | DYKDDDDK (SEQ ID NO: 395) |
| | DLSRHIRTHTGEKPFQCDICMRNFSQSG | NES |
| | DLTRHIRTHTGSEKPFQCDICMRNFSRSD | VDEMTKKFGTLTIHDTEK (SEQ ID NO: 396) |
| | SLSAHIRTHTGEKPFQCDICMRNFSQKA | Linker |
| | TRITHTKIHLRGSGGGGSGGSGGSGSYA | GS |
| | LGPYQISAPQLPAYNGQTVGTFYYVND | NES2 |
| | AGGLESKVFSSGGPTPYPNYANAGHVE | LqKKLEELELD (SEQ ID NO: 397) |
| | GQSALFMRDNGISEGLVFHNNPEGTCGF | Linker |
| | CVNMTETLLPENAKMTVVPPEGSGGST | AA |
| | NLSDIIEKETGKQLVIQESILMLPEEVEEV | ZF (R13-1) |
| | IGNKPESDILVHTAYDESTDENVMLLTS | MAERPFQCDICMRNFSRSDNLSTHIRTHTGEKP |
| | DAPEYKPWALVIQDSNGENKIKMLGSG | FQCDICMRNFSDRSDLSRHIRTHTGEKPFQCDIC |
| | ATNFSLLKQAGDVEENPGPMASVLTPLL | MRNFSQSGDLTRHIRTHTGSEKPFQCDICMRNF |
| | LRGLTGSARRLPVPRAKIHSLGSTNLSDI | SRSDSLSAHIRTHTGEKPFQCDICMRNFSQKAT |
| | IEKETGKQLVIQESILMLPEEVEEVIGNK | RITHTKIHLR (SEQ ID NO: 404) |
| | PESDILVHTAYDESTDENVMLLTSDAPE | Linker |
| | YKPWALVIQDSNGENKIKML (SEQ ID | GSGGGGSGGSGGS (SEQ ID NO: 399) |
| | NO: 385) | Split DddA (DddA-G1397N) |
| | | GSYALGPYQISAPQLPAYNGQTVGTFYYVNDA |
| | | GGLESKVFSSGGPTPYPNYANAGHVEGQSALF |
| | | MRDNGISEGLVFHNNPEGTCGFCVNMTETLLP |
| | | ENAKMTVVPPEG (SEQ ID NO: 284) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| | | Linker |
| | | GSG |
| | | P2A Peptide |
| | | ATNFSLLKQAGDVEENPGP (SEQ ID NO: 400) |
| | | MTS |
| | | MASVLTPLLLRGLTGSARRLPVPRAKIHSL |
| | | (SEQ ID NO: 401) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| 5xZnF-9- | MLGFVGRVAAAPASGALRRLTPSASLPP | MTS |
| R13 v6 | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 394) |
| | LEELELDAAMAERPFQCDICMRNFSQSS | FLAG tag |
| | SLVRHIRTHTGEKPFQCDICMRNFSRSD | DYKDDDDK (SEQ ID NO: 395) |
| | NLVRHIRTHTGSEKPFQCDICMRNFSQA | NES |
| | GHLASHIRTHTGEKPFQCDICMRNFSRK | VDEMTKKFGTLTIHDTEK (SEQ ID NO: 396) |
| | DNLKNHIRTHTGEKPFQCDICMRNFSRK | Linker |
| | DALRGHTKIHLRGSGGGGSGGSGGSAIP | GS |
| | VKRGATGETKVFTGNSNSPKSPTKGGCS | NES2 |
| | GGSTNLSDIIEKETGKQLVIQESILMLPEE | LQKKLEELELD (SEQ ID NO: 397) |
| | VEEVIGNKPESDILVHTAYDESTDENVM | Linker |
| | LLTSDAPEYKPWALVIQDSNGENKIKML | AA |
| | GSGATNFSLLKQAGDVEENPGPMASVL | ZF (5xZnF-9-R13) |
| | TPLLLRGLTGSARRLPVPRAKIHSLGSTN | MAERPFQCDICMRNFSQSSSLVRHIRTHTGEKP |
| | LSDIIEKETGKQLVIQESILMLPEEVEEVI | FQCDICMRNFSRSDNLVRHIRTHTGSEKPFQCDI |
| | GNKPESDILVHTAYDESTDENVMLLTSD | CMRNFSQAGHLASHIRTHTGEKPFQCDICMRNF |
| | APEYKPWALVIQDSNGENKIKML (SEQ | SRKDNLKNHIRTHTGEKPFQCDICMRNFSRKDA |
| | ID NO: 386) | LRGHTKIHLR (SEQ ID NO: 405) |
| | | Linker |
| | | GSGGGGSGGSGGS (SEQ ID NO: 399) |
| | | Split DddA (DddA-G1397C) |
| | | AIPVKRGATGETKVFTGNSNSPKSPTKGGC |
| | | (SEQ ID NO: 286) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| | | Linker |
| | | GSG |
| | | P2A Peptide |
| | | ATNFSLLKQAGDVEENPGP (SEQ ID NO: 400) |
| | | MTS |
| | | MASVLTPLLLRGLTGSARRLPVPRAKIHSL |
| | | (SEQ ID NO: 401) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| 5xZnF-12- | MLGFVGRVAAAPASGALRRLTPSASLPP | MTS |
| R13 v6 | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 394) |
| | LEELELDAAMAERPFQCDICMRNFSRSD | FLAG tag |
| | HLTTHIRTHTGEKPFQCDICMRNFSQSSS | DYKDDDDK (SEQ ID NO: 395) |
| | LVRHIRTHTGSEKPFQCDICMRNFSRSD | NES |
| | NLVRHIRTHTGEKPFQCDICMRNFSQAG | VDEMTKKFGTLTIHDTEK (SEQ ID NO: 396) |
| | HLASHIRTHTGEKPFQCDICMRNFSRKD | Linker |
| | NLKNHTKIHLRGSGGGGSGGSGGSAIPV | GS |
| | KRGATGETKVFTGNSNSPKSPTKGGCSG | NES2 |
| | GSTNLSDIIEKETGKQLVIQESILMLPEEV | LqKKLEELELD (SEQ ID NO: 397) |
| | EEVIGNKPESDILVHTAYDESTDENVML | Linker |
| | LTSDAPEYKPWALVIQDSNGENKIKML | AA |
| | GSGATNFSLLKQAGDVEENPGPMASVL | ZF (5xZnF-12-R13) |
| | TPLLLRGLTGSARRLPVPRAKIHSLGSTN | MAERPFQCDICMRNFSRSDHLTTHIRTHTGEKP |
| | LSDIIEKETGKQLVIQESILMLPEEVEEVI | FQCDICMRNFSQSSSLVRHIRTHTGSEKPFQCDI |
| | GNKPESDILVHTAYDESTDENVMLLTSD | CMRNFSRSDNLVRHIRTHTGEKPFQCDICMRNF |
| | APEYKPWALVIQDSNGENKIKML (SEQ | SQAGHLASHIRTHTGEKPFQCDICMRNFSRKDN |
| | ID NO: 387) | LKNHTKIHLR (SEQ ID NO: 406) |
| | | Linker |
| | | GSGGGGSGGSGGS (SEQ ID NO: 399) |
| | | Split DddA (DddA-G1397C) |
| | | AIPVKRGATGETKVFTGNSNSPKSPTKGGC |
| | | (SEQ ID NO: 286) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| | | Linker |
| | | GSG |
| | | P2A Peptide |
| | | ATNFSLLKQAGDVEENPGP (SEQ ID NO: 400) |
| | | MTS |
| | | MASVLTPLLLRGLTGSARRLPVPRAKIHSL |
| | | (SEQ ID NO: 401) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| R8 v3 | MLGFVGRVAAAPASGALRRLTPSASLPP | MTS |
| | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 394) |
| | PFQCRICMRNFSTSGSLSRHIRTHTGEKP | FLAG tag |
| | FACDICGRKFAQSGSLTRHTKIHTGGQR | DYKDDDDK (SEQ ID NO: 395) |
| | PFQCRICMRNFSRSDALSQHIRTHTGEKP | NES |
| | FACDICGRKFARNDNRITHTKIHTGEKPF | VDEMTKKFGTLTIHDTEK (SEQ ID NO: 396) |
| | QCRICMRKFARSDHLTQHTKIHLRGSGG | Linker |
| | GGSGGSGGSGSYALGPYQISAPQLPAYN | AA |
| | GQTVGTFYYVNDAGGLESKVFSSGGPT | ZF (R8) |
| | PYPNYANAGHVEGQSALFMRDNGISEG | MAERPFQCRICMRNFSTSGSLSRHIRTHTGEKPF |
| | LVFHNNPEGTCGFCVNMTETLLPENAK | ACDICGRKFAQSGSLTRHTKIHTGGQRPFQCRI |
| | MTVVPPEGSGGSTNLSDIIEKETGKQLVI | CMRNFSRSDALSQHIRTHTGEKPFACDICGRKF |
| | QESILMLPEEVEEVIGNKPESDILVHTAY | ARNDNRITHTKIHTGEKPFQCRICMRKFARSDH |
| | DESTDENVMLLTSDAPEYKPWALVIQD | LTQHTKIHLR (SEQ ID NO: 407) |
| | SNGENKIKML (SEQ ID NO: 388) | Linker |
| | | GSGGGGSGGSGGS (SEQ ID NO: 399) |
| | | Split DddA (DddA-G1397N) |
| | | GSYALGPYQISAPQLPAYNGQTVGTFYYVNDA |
| | | GGLESKVFSSGGPTPYPNYANAGHVEGQSALF |
| | | MRDNGISEGLVFHNNPEGTCGFCVNMTETLLP |
| | | ENAKMTVVPPEG (SEQ ID NO: 284) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| 5xZnF-4- | MLGFVGRVAAAPASGALRRLTPSASLPP | MTS |
| R8 v3 | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 394) |
| | PFQCRICMRNFSQASNLISHIRTHTGEKP | FLAG tag |
| | FACDICGRKFATSHSLTEHTKIHTGSQKP | DYKDDDDK (SEQ ID NO: 395) |
| | FQCRICMRNFSERSHLREHIRTHTGEKPF | NES |
| | ACDICGRKFAQSGNLTEHTKIHTGEKPF | VDEMTKKFGTLTIHDTEK (SEQ ID NO: 396) |
| | QCRICMRKFASKKALTEHTKIHLRGSGG | Linker |
| | GGSGGSGGSAIPVKRGATGETKVFTGNS | AA |
| | NSPKSPTKGGCSGGSTNLSDIIEKETGKQ | ZF (5xZnF-4-R8) |
| | LVIQESILMLPEEVEEVIGNKPESDILVHT | MAERPFQCRICMRNFSQASNLISHIRTHTGEKPF |
| | AYDESTDENVMLLTSDAPEYKPWALVI | ACDICGRKFATSHSLTEHTKIHTGSQKPFQCRIC |
| | QDSNGENKIKML (SEQ ID NO: 389) | MRNFSERSHLREHIRTHTGEKPFACDICGRKFA |
| | | QSGNLTEHTKIHTGEKPFQCRICMRKFASKKAL |
| | | TEHTKIHLR (SEQ ID NO: 408) |
| | | Linker |
| | | GSGGGGSGGSGGS (SEQ ID NO: 399) |
| | | Split DddA (DddA-G1397C) |
| | | AIPVKRGATGETKVFTGNSNSPKSPTKGGC |
| | | (SEQ ID NO: 286) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| 5xZnF-10- | MLGFVGRVAAAPASGALRRLTPSASLPP | MTS |
| R8 v3 | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 394) |
| | PFQCRICMRNFSQASNLISHIRTHTGEKP | FLAG tag |
| | FACDICGRKFAQRANLRAHTKIHTGSQK | DYKDDDDK (SEQ ID NO: 395) |
| | PFQCRICMRNFSQASNLISHIRTHTGEKP | NES |
| | FACDICGRKFATSHSLTEHTKIHTGEKPF | VDEMTKKFGTLTIHDTEK (SEQ ID NO: 396) |
| | QCRICMRKFAERSHLREHTKIHLRGSGG | Linker |
| | GGSGGSGGSAIPVKRGATGETKVFTGNS | AA |
| | NSPKSPTKGGCSGGSTNLSDIIEKETGKQ | ZF (5xZnF-10-R8) |
| | LVIQESILMLPEEVEEVIGNKPESDILVHT | MAERPFQCRICMRNFSQASNLISHIRTHTGEKPF |
| | AYDESTDENVMLLTSDAPEYKPWALVI | ACDICGRKFAQRANLRAHTKIHTGSQKPFQCRI |
| | QDSNGENKIKML (SEQ ID NO: 390) | CMRNFSQASNLISHIRTHTGEKPFACDICGRKFA |
| | | TSHSLTEHTKIHTGEKPFQCRICMRKFAERSHLR |
| | | EHTKIHLR (SEQ ID NO: 409) |
| | | Linker |
| | | GSGGGGSGGSGGS (SEQ ID NO: 399) |
| | | Split DddA (DddA-G1397C) |
| | | AIPVKRGATGETKVFTGNSNSPKSPTKGGC |
| | | (SEQ ID NO: 286) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| R13-1 v3 | MLGFVGRVAAAPASGALRRLTPSASLPP | MTS |
| | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 394) |
| | PFQCRICMRNFSRSDNLSTHIRTHTGEKP | FLAG tag |
| | FACDICGRKFADRSDLSRHTKIHTGEKPF | DYKDDDDK (SEQ ID NO: 395) |
| | QCRICMRKFAQSGDLTRHTKIHTGSQKP | NES |
| | FQCRICMRNFSRSDSLSAHIRTHTGEKPF | VDEMTKKFGTLTIHDTEK (SEQ ID NO: 396) |
| | ACDICGRKFAQKATRITHTKIHLRGSGG | Linker |
| | GGSGGSGGSGSYALGPYQISAPQLPAYN | AA |
| | GQTVGTFYYVNDAGGLESKVFSSGGPT | ZF (R13-1) |
| | PYPNYANAGHVEGQSALFMRDNGISEG | MAERPFQCRICMRNFSRSDNLSTHIRTHTGEKP |
| | LVFHNNPEGTCGFCVNMTETLLPENAK | FACDICGRKFADRSDLSRHTKIHTGEKPFQCRIC |
| | MTVVPPEGSGGSTNLSDIIEKETGKQLVI | MRKFAQSGDLTRHTKIHTGSQKPFQCRICMRNF |
| | QESILMLPEEVEEVIGNKPESDILVHTAY | SRSDSLSAHIRTHTGEKPFACDICGRKFAQKAT |
| | DESTDENVMLLTSDAPEYKPWALVIQD | RITHTKIHLR (SEQ ID NO: 410) |
| | SNGENKIKML (SEQ ID NO: 391) | Linker |
| | | GSGGGGSGGSGGS (SEQ ID NO: 399) |
| | | Split DddA (DddA-G1397N) |
| | | GSYALGPYQISAPQLPAYNGQTVGTFYYVNDA |
| | | GGLESKVFSSGGPTPYPNYANAGHVEGQSALF |
| | | MRDNGISEGLVFHNNPEGTCGFCVNMTETLLP |
| | | ENAKMTVVPPEG (SEQ ID NO: 284) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| 5xZnF-9- | MLGFVGRVAAAPASGALRRLTPSASLPP | MTS |
| R13 v3 | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 394) |
| | PFQCRICMRNFSQSSSLVRHIRTHTGEKP | FLAG tag |
| | FACDICGRKFARSDNLVRHTKIHTGSQK | DYKDDDDK (SEQ ID NO: 395) |
| | PFQCRICMRNFSQAGHLASHIRTHTGEK | NES |
| | PFACDICGRKFARKDNLKNHTKIHTGEK | VDEMTKKFGTLTIHDTEK (SEQ ID NO: 396) |
| | PFQCRICMRKFARKDALRGHTKIHLRGS | Linker |
| | GGGGSGGSGGSAIPVKRGATGETKVFT | AA |
| | GNSNSPKSPTKGGCSGGSTNLSDIIEKET | ZF (5xZnF-9-R13) |
| | GKQLVIQESILMLPEEVEEVIGNKPESDI | MAERPFQCRICMRNFSQSSSLVRHIRTHTGEKP |
| | LVHTAYDESTDENVMLLTSDAPEYKPW | FACDICGRKFARSDNLVRHTKIHTGSQKPFQCR |
| | ALVIQDSNGENKIKML (SEQ ID NO: 392) | ICMRNFSQAGHLASHIRTHTGEKPFACDICGRK |
| | | FARKDNLKNHTKIHTGEKPFQCRICMRKFARK |
| | | DALRGHTKIHLR (SEQ ID NO: 411) |
| | | Linker |
| | | GSGGGGSGGSGGS (SEQ ID NO: 399) |
| | | Split DddA (DddA-G1397C) |
| | | AIPVKRGATGETKVFTGNSNSPKSPTKGGC |
| | | (SEQ ID NO: 286) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |
| 5xZnF-12- | MLGFVGRVAAAPASGALRRLTPSASLPP | MTS |
| R13 v3 | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 394) |
| | PFQCRICMRNFSRSDHLTTHIRTHTGEKP | FLAG tag |
| | FACDICGRKFAQSSSLVRHTKIHTGSQKP | DYKDDDDK (SEQ ID NO: 395) |
| | FQCRICMRNFSRSDNLVRHIRTHTGEKP | NES |
| | FACDICGRKFAQAGHLASHTKIHTGEKP | VDEMTKKFGTLTIHDTEK (SEQ ID NO: 396) |
| | FQCRICMRKFARKDNLKNHTKIHLRGSG | Linker |
| | GGGSGGSGGSAIPVKRGATGETKVFTG | AA |
| | NSNSPKSPTKGGCSGGSTNLSDIIEKETG | ZF (5xZnF-12-R13) |
| | KQLVIQESILMLPEEVEEVIGNKPESDIL | MAERPFQCRICMRNFSRSDHLTTHIRTHTGEKP |
| | VHTAYDESTDENVMLLTSDAPEYKPWA | FACDICGRKFAQSSSLVRHTKIHTGSQKPFQCRI |
| | LVIQDSNGENKIKML (SEQ ID NO: 393) | CMRNFSRSDNLVRHIRTHTGEKPFACDICGRKF |
| | | AQAGHLASHTKIHTGEKPFQCRICMRKFARKD |
| | | NLKNHTKIHLR (SEQ ID NO: 412) |
| | | Linker |
| | | GSGGGGSGGSGGS (SEQ ID NO: 399) |
| | | Split DddA (DddA-G1397C) |
| | | AIPVKRGATGETKVFTGNSNSPKSPTKGGC |
| | | (SEQ ID NO: 286) |
| | | Linker |
| | | SGGS (SEQ ID NO: 348) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 341) |

### Optimisation of the ZF Scaffold Sequence Encompasses Two Separate Improvements

First, ZFs designed according to the Zif268 scaffold consisted of an N-terminal sequence (MAERP (SEQ ID NO: 421)), followed by a number of ZF repeats separated by linkers, and ended with a C-terminal sequence (HTKIHLR (SEQ ID NO: 422)). ZFs are typically composed of multiple ZF repeats, and each repeat will have a N-terminal scaffold half (FQCRICMRNFS (SEQ ID NO: 423) or FACDICGRKFA (SEQ ID NO: 424) or FQCRICMRKFA (SEQ ID NO: 425), a seven amino acid DNA-binding region, a C-terminal scaffold half (HIRTH (SEQ ID NO: 426) or HTKIH (SEQ ID NO: 427), and a linker (TGEKP (SEQ ID NO: 428) or TGQKP (SEQ ID NO: 429), or TGSQKP (SEQ ID NO: 430)). An example of this structure is noted below:

(SEQ ID NO: 421)
MAERP
(SEQ ID NO: 431)
FQCRICMRNFS...ZF1...HIRTH TGEKP
(SEQ ID NO: 432)
FACDICGRKFA...ZF2...HTKIH TGSQKP
(SEQ ID NO: 431)
FQCRICMRNFS...ZF3...HIRTH TGEKP
(SEQ ID NO: 433)
FACDICGRKFA...ZF4...HTKIH TGEKP
(SEQ ID NO: 434)
FQCRICMRKFA...ZF5...HTKIHLR

Wherein . . . ZF # . . . was replaced with seven amino acids (XXXXXXX) which specify the 3 bp DNA sequence to which this ZF repeat binds.

Improvements in ZF-BE editing efficiencies were only found in sequences that were scanned computationally to encode a weaker NLS. This implies designing ZFs such that every repeat will have an N-terminal scaffold half of FQCRICMRNFS (SEQ ID NO: 423) only, the seven amino acid DNA-binding region, a C-terminal scaffold half of HIRTH (SEQ ID NO: 426) only, and a linker of TGEKP (SEQ ID NO: 428) or TGSEKP (SEQ ID NO: 435) only. This strategy reduces the number of positively-charged residues (particularly Lys, K) which contribute to the inherent NLS of ZFs. As a result, the above example sequence would be converted into the sequence below, which produced improved levels of mitochondrial BE:

(SEQ ID NO: 421)
MAERP
(SEQ ID NO: 431)
FQCRICMRNFS...ZF1...HIRTH TGEKP
(SEQ ID NO: 436)
FQCRICMRNFS...ZF2...HIRTH TGSEKP
(SEQ ID NO: 431)
FQCRICMRNFS...ZF3...HIRTH TGEKP
(SEQ ID NO: 431)
FQCRICMRNFS...ZF4...HIRTH TGEKP
(SEQ ID NO: 437)
FQCRICMRNFS...ZF5...HTKIHLR

Second, within each ZF repeat, mutation of the FQCRICMRNFS (SEQ ID NO: 423)N-terminal scaffold half to FQCDICMRNFS (SEQ ID NO: 438) (a single R to D mutation in each repeat), removes a positively-charged amino acid and replaces it with a negatively-charged amino acid. This weakens the NLS inherently encoded within ZFs without disrupting its ability to bind to DNA.

In combination, these improvements are significant because they specify a way to construct not just ZFs but specifically mitochondrially-optimised ZFs for the first time. Previously, ZFs have been used for nuclear genome editing, or repurposed without any optimization (beyond addition of an MTS) for mitochondrial genome editing. However, these advancements represent optimisations being made to ZFs to specifically tailor their architecture towards mitochondrial genome editing—improving their activity by boosting mitochondrial localization and reducing nuclear localization without impairing the DNA-binding ability of the ZFs themselves.

### Co-Expression and Targeting of an Additional Copy of UGI to the Mitochondria in Trans

Previous BEs rely on inhibition of UDG, either in the nucleus or mitochondria, by fusion of one or multiple copies of UGI to the BE protein itself and act via colocalization. However, co-expression of additional copies of UGI as separate polypeptides (targeted to mitochondria via their own MTS) in order to suppress mitochondrial UDG to even lower levels has not previously been reported. This is an additional way to suppress mitochondrial UDG activity which can be applied to any mitochondrial BE (ZF- or TALE-based).

### Example 6. Tale-DdCBE Mitochondrial Base Editing with Alternative UGI Homologs

New UGI homologs were identified by computational search for homology to known UGI proteins, namely from bacteriophage PBS2, bacteriophage phi29 and S. aureus, in addition to literature review (see UGI2 below). These newly-identified UGI homologs were tested for their ability to inhibit mitochondrial UDG in the context of TALE-DdCBEs (FIG. 88). Three different sites were chosen, reflecting high (ND4), medium (ND5.1) and low (ATP8) editing efficiencies to establish if the observed effects were consistent across different sites (FIG. 89). The best performing homologs were UG15 and UGI 17, and lead to robust improvements in editing efficiencies across different sites in the context of TALE-DdCBE (FIGS. 90A-90C) Five UGI homologs were deemed to show relative improvements over the canonical UGI (from bacteriophage PBS2) typically used in BE experiments (FIGS. 90A-90D). The following sequences were used in the context of TALE-DdCBEs, replacing the normal (canonical) UGI sequence following the DddA split deaminase and SGGS linker as shown in FIG. 90D. Note that UGI12 was predicted to exist as a homodimer and to bind to UDG in this homodimeric form. Therefore, UGI12 was tested as two UGI12 monomers linked in tandem together via a flexible linker. However, given the predicted nature of the homodimeric structure (each monomer binding antiparallel to the other), it was required to fuse one monomer of UGI12 to a circularly-permuted version of another monomer of UGI12.

| UGI | Sequence | SEQ ID NO: |
|---|---|---|
| Canonical UGI | TNLSDIIEKETGKQLVIQESILMLPEEVEEVIGNKPESDILVHTAYDE | 341 |
| | STDENVMLLTSDAPEYKPWALVIQDSNGENKIKML | |
| UGI2 | MTLELQLKHYITNLFNLPKDEKWHCESIEEIADDILPDQYVRLGAL | 445 |
| | SNKILQTYTYYSDTLHESNIYPFILYYQKQLIAIGYIDENHDMDFLY | |
| | LHNTIMPLLDQRYLLTGGQ | |
| UGI 3 | MNKNFDEVKADLRTVTGKKIEFKERLKNILRVQMNQLGFEDSYMI | 446 |
| | QVQVSSDQEEWVECHENMSLSDFEVMYGNISGEIKRMTVVKYEE | |
| | ANIEKLVELKFEYEYAKAHQEYIRAYTKLMSNTLYGRKPSL | |
| UGI5 | MNEEKMHYRDAIKEVELTMMSLDSHFRTHKEFTDSYLLVLILEDV | 447 |
| | VGETRVEVSEGLTFDEASYIIGGTSDNILNMHMINYCEKNREEIYK | |
| | WLKVSRVNTFKSNYAKMLLNTAYGKDLLKGVVK | |
| UGI7 | MNNHFMSIGRNCSKCNNVRLNEDFSKSEEICNECFDKEERFVDSY | 375 |
| | TLIYITEDETGKRFEAILENQTIEETEIIYGNIIDKIIVWNVILTM | |
| UGI12 | DGNEHWEVHPGLSLSDFEVVYGNNPHQIVKLRLDKEVGGSGGSM | 376 |
| | VQNDFIDSYTLCWLLRDDSGGGGSMVQNDFIDSYTLCWLLRDDD | |
| | GNEHWEVHPGLSLSDFEVVYGNNPHQIVKLRLDKEV | |
| UGI12 | DGNEHWEVHPGLSLSDFEVVYGNNPHQIVKLRLDKEV | 439 |
| Monomer 1-C | | |
| UGI12 | GGSGGS | 440 |
| Monomer 1-C | | |
| Linker | | |
| UGI12 | MVQNDFIDSYTLCWLLRDD | 441 |
| Monomer 2-N | | |
| UGI12 | SGGGGS | 442 |
| Monomer 2-N | | |
| Linker | | |
| UGI12 | MVQNDFIDSYTLCWLLRDDDGNEHWEVHPGLSLSDFEVVYGNNP | 443 |
| Monomer 2 | HQIVKLRLDKEV | |

Three of the top perfuming UGI homologs were tested in the context of BE4 max. The remaining two were unable to be cloned in the BE4max architecture as they were toxic to E. coli in UGI-UGI (BE4max) architecture. However, results show that the UGI homologs did not improve editing efficiency in BE4max (FIGS. 91A-91D.)

### REFERENCES

The following references are each incorporated herein by reference in their entireties.
1. Jinek, M. et al. A Programmable Dual-RNA-Guided DNA Endonuclease in Adaptive Bacterial Immunity. Science 337, 816-821 (2012).
2. Cong, L. et al. Multiplex Genome Engineering Using CRISPR/Cas Systems. Science 339, 819-823 (2013).
3. Komor, A. C., Badran, A. H. & Liu, D. R. CRISPR-Based Technologies for the Manipulation of Eukaryotic Genomes. Cell 168, 20-36 (2017).
4. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
5. Nishida, K. et al. Targeted nucleotide editing using hybrid prokaryotic and vertebrate adaptive immune systems. Science 353, aaf8729 (2016).
6. Gaudelli, N. M. et al. Programmable base editing of A·T to G·C in genomic DNA without DNA cleavage. Nature 551, 464-471 (2017).
7. ClinVar, July 2019.
8. Dunbar, C. E. et al. Gene therapy comes of age. Science 359, eaan4672 (2018).
9. Cox, D. B. T., Platt, R. J. & Zhang, F. Therapeutic genome editing: prospects and challenges. Nat. Med. 21, 121-131 (2015).
10. Adli, M. The CRISPR tool kit for genome editing and beyond. Nat. Commun. 9, 1911 (2018).
11. Kleinstiver, B. P. et al. Engineered CRISPR-Cas9 nucleases with altered PAM specificities. Nature 523, 481-485 (2015).
12. Kleinstiver, B. P. et al. High-fidelity CRISPR-Cas9 nucleases with no detectable genome-wide off-target effects. Nature 529, 490-495 (2016).
13. Hu, J. H. et al. Evolved Cas9 variants with broad PAM compatibility and high DNA specificity. Nature 556, 57-63 (2018).
14. Nishimasu, H. et al. Engineered CRISPR-Cas9 nuclease with expanded targeting space. Science 361, 1259-1262 (2018).
15. Jasin, M. & Rothstein, R. Repair of strand breaks by homologous recombination. Cold Spring Harb. Perspect. Biol. 5, a012740 (2013).
16. Paquet, D. et al. Efficient introduction of specific homozygous and heterozygous mutations using CRISPR/Cas9. Nature 533, 125-129 (2016).
17. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
18. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
19. Ihry, R. J. et al. p53 inhibits CRISPR-Cas9 engineering in human pluripotent stem cells. Nat. Med. 24, 939-946 (2018).
20. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat. Biotechnol. 34, 339-344 (2016).
21. Srivastava, M. et al. An Inhibitor of Nonhomologous End-Joining Abrogates Double-Strand Break Repair and Impedes Cancer Progression. Cell 151, 1474-1487 (2012).
22. Chu, V. T. et al. Increasing the efficiency of homology-directed repair for CRISPR-Cas9-induced precise gene editing in mammalian cells. Nat. Biotechnol. 33, 543-548 (2015).
23. Maruyama, T. et al. Increasing the efficiency of precise genome editing with CRISPR-Cas9 by inhibition of nonhomologous end joining. Nat. Biotechnol. 33, 538-542 (2015).
24. Kim, Y. B. et al. Increasing the genome-targeting scope and precision of base editing with engineered Cas9-cytidine deaminase fusions. Nat. Biotechnol. 35, 371-376 (2017).
25. Li, X. et al. Base editing with a Cpf1-cytidine deaminase fusion. Nat. Biotechnol. 36, 324-327 (2018).
26. Gehrke, J. M. et al. An APOBEC3A-Cas9 base editor with minimized bystander and off-target activities. Nat. Biotechnol. (2018). doi:10.1038/nbt.4199
27. Rees, H. A. & Liu, D. R. Base editing: precision chemistry on the genome and transcriptome of living cells. Nat. Rev. Genet. 1 (2018). doi:10.1038/s41576-018-0059-1.
28. Ostertag, E. M. & Kazazian Jr, H. H. Biology of Mammalian L1 Retrotransposons. Annu. Rev. Genet. 35, 501-538 (2001).
29. Zimmerly, S., Guo, H., Perlman, P. S. & Lambowltz, A. M. Group II intron mobility occurs by target DNA-primed reverse transcription. Cell 82, 545-554 (1995).
30. Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
31. Feng, Q., Moran, J. V., Kazazian, H. H. & Boeke, J. D. Human L1 retrotransposon encodes a conserved endonuclease required for retrotransposition. Cell 87, 905-916 (1996).
32. Jinek, M. et al. Structures of Cas9 Endonucleases Reveal RNA-Mediated Conformational Activation. Science 343, 1247997 (2014).
33. Jiang, F. et al. Structures of a CRISPR-Cas9 R-loop complex primed for DNA cleavage. Science aad8282 (2016). doi:10.1126/science.aad8282
34. Qi, L. S. et al. Repurposing CRISPR as an RNA-Guided Platform for Sequence-Specific Control of Gene Expression. Cell 152, 1173-1183 (2013).
35. Tang, W., Hu, J. H. & Liu, D. R. Aptazyme-embedded guide RNAs enable ligand-responsive genome editing and transcriptional activation. Nat. Commun. 8, 15939 (2017).
36. Shechner, D. M., Hacisuleyman, E., Younger, S. T. & Rinn, J. L. Multiplexable, locus-specific targeting of long RNAs with CRISPR-Display. Nat. Methods 12, 664-670 (2015).
37. Anders, C. & Jinek, M. Chapter One—In vitro Enzymology of Cas9. in Methods in Enzymology (eds. Doudna, J. A. & Sontheimer, E. J.) 546, 1-20 (Academic Press, 2014).
38. Briner, A. E. et al. Guide RNA Functional Modules Direct Cas9 Activity and Orthogonality. Mol. Cell 56, 333-339 (2014).
39. Nowak, C. M., Lawson, S., Zerez, M. & Bleris, L. Guide RNA engineering for versatile Cas9 functionality. Nucleic Acids Res. 44, 9555-9564 (2016).
40. Sternberg, S. H., Redding, S., Jinek, M., Greene, E. C. & Doudna, J. A. DNA interrogation by the CRISPR RNA-guided endonuclease Cas9. Nature 507, 62-67 (2014).
41. Mohr, S. et al. Thermostable group II intron reverse transcriptase fusion proteins and their use in cDNA synthesis and next-generation RNA sequencing. RNA 19, 958-970 (2013).
42. Stamos, J. L., Lentzsch, A. M. & Lambowitz, A. M. Structure of a Thermostable Group II Intron Reverse Transcriptase with Template-Primer and Its Functional and Evolutionary Implications. Mol. Cell 68, 926-939.e4 (2017).
43. Zhao, C. & Pyle, A. M. Crystal structures of a group II intron maturase reveal a missing link in spliceosome evolution. Nat. Struct. Mol. Biol. 23, 558-565 (2016).
44. Zhao, C., Liu, F. & Pyle, A. M. An ultraprocessive, accurate reverse transcriptase encoded by a metazoan group II intron. RNA 24, 183-195 (2018).
45. Ran, F. A. et al. Genome engineering using the CRISPR-Cas9 system. Nat. Protoc. 8, 2281-2308 (2013).
46. Liu, Y., Kao, H.-I. & Bambara, R. A. Flap endonuclease 1: a central component of DNA metabolism. Annu. Rev. Biochem. 73, 589-615 (2004).
47. Krokan, H. E. & Bjsris, M. Base Excision Repair. Cold Spring Harb. Perspect. Biol. 5, (2013).
48. Kelman, Z. PCNA: structure, functions and interactions. Oncogene 14, 629-640 (1997).
49. Choe, K. N. & Moldovan, G.-L. Forging Ahead through Darkness: PCNA, Still the Principal Conductor at the Replication Fork. Mol. Cell 65, 380-392 (2017).
50. Li, X., Li, J., Harrington, J., Lieber, M. R. & Burgers, P. M. Lagging strand DNA synthesis at the eukaryotic replication fork involves binding and stimulation of FEN-1 by proliferating cell nuclear antigen. J. Biol. Chem. 270, 22109-22112 (1995).
51. Tom, S., Henricksen, L. A. & Bambara, R. A. Mechanism whereby proliferating cell nuclear antigen stimulates flap endonuclease 1. J. Biol. Chem. 275, 10498-10505 (2000).
52. Tanenbaum, M. E., Gilbert, L. A., Qi, L. S., Weissman, J. S. & Vale, R. D. A protein-tagging system for signal amplification in gene expression and fluorescence imaging. Cell 159, 635-646 (2014).
53. Bertrand, E. et al. Localization of ASH1 mRNA particles in living yeast. Mol. Cell 2, 437-445 (1998).
54. Dahlman, J. E. et al. Orthogonal gene knockout and activation with a catalytically active Cas9 nuclease. Nat. Biotechnol. 33, 1159-1161 (2015).
55. Tsai, S. Q. et al. GUIDE-seq enables genome-wide profiling of off-target cleavage by CRISPR-Cas nucleases. Nat. Biotechnol. 33, 187-197 (2015).
56. Tsai, S. Q. et al. CIRCLE-seq: a highly sensitive in vitro screen for genome-wide CRISPR-Cas9 nuclease off-targets. Nat. Methods 14, 607-614 (2017).
57. Schek N, Cooke C, Alwine J C. Molecular and Cellular Biology. (1992).
58. Gil A, Proudfoot N J. Cell. (1987).
59. Zhao, B. S., Roundtree, I. A., He, C. Nat Rev Mol Cell Biol. (2017).
60. Rubio, M. A. T., Hopper, A. K. Wiley Interdiscip Rev RNA (2011).
61. Shechner, D. M., Hacisuleyman E., Younger, S. T., Rinn, J. L. Nat Methods. (2015).
62. Paige, J. S., Wu, K. Y., Jaffrey, S.R. Science (2011).
63. Ray D., . . . Hughes T R. Nature (2013).
64. Chadalavada, D. M., Cerrone-Szakal, A. L., Bevilacqua, P. C. RNA (2007).
65. Forster A C, Symons R H. Cell. (1987).
66. Weinberg Z, Kim P B, Chen T H, Li S, Harris K A, Lunse C E, Breaker R R. Nat. Chem. Biol. (2015).
67. Feldstein P A, Buzayan J M, Bruening G. Gene (1989).
68. Saville B J, Collins R A. Cell. (1990).
69. Winkler W C, Nahvi A, Roth A, Collins J A, Breaker R R. Nature (2004).
70. Roth A, Weinberg Z, Chen A G, Kim P G, Ames T D, Breaker R R. Nat Chem Biol. (2013).
71. Choudhury R, Tsai Y S, Dominguez D, Wang Y, Wang Z. Nat Commun. (2012).
72. MacRae I J, Doudna J A. Curr Opin Struct Biol. (2007).
73. Bernstein E, Caudy A A, Hammond S M, Hannon G J Nature (2001).
74. Filippov V, Solovyev V, Filippova M, Gill SS. Gene (2000).
75. Cadwell R C and Joyce G F. PCR Methods Appl. (1992).
76. McInerney P, Adams P, and Hadi M Z. Mol Biol Int. (2014).
77. Esvelt K M, Carlson J C, and Liu D R. Nature. (2011).
78. Naorem S S, Hin J, Wang S, Lee W R, Heng X, Miller J F, Guo H. Proc Natl Acad Sci USA (2017).
79. Martinez M A, Vartanian J P, Wain-Hobson S. Proc Natl Acad Sci USA (1994).
80. Meyer A J, Ellefson J W, Ellington A D. Curr Protoc Mol Biol. (2014).
81. Wang H H, Isaacs F J, Carr P A, Sun Z Z, Xu G, Forest C R, Church G M. Nature. (2009).
82. Nyerges Á et al. Proc Natl Acad Sci USA. (2016).
83. Mascola J R, Haynes B F. Immunol Rev. (2013).
84. X. Wen, K. Wen, D. Cao, G. Li, R. W. Jones, J. Li, S. Szu, Y. Hoshino, L. Yuan, Inclusion of a universal tetanus toxoid CD4(+) T cell epitope P2 significantly enhanced the immunogenicity of recombinant rotavirus ΔVP8* subunit parenteral vaccines. Vaccine 32, 4420-4427 (2014).
85. G. Ada, D. Isaacs, Carbohydrate-protein conjugate vaccines. Clin Microbiol Infect 9, 79-85 (2003).
86. E. Malito, B. Bursulaya, C. Chen, P. L. Surdo, M. Picchianti, E. Balducci, M. Biancucci, A. Brock, F. Berti, M. J. Bottomley, M. Nissum, P. Costantino, R. Rappuoli, G. Spraggon, Structural basis for lack of toxicity of the diphtheria toxin mutant CRM197. Proceedings of the National Academy of Sciences 109, 5229 (2012).
87. J. de Wit, M. E. Emmelot, M. C. M. Poelen, J. Lanfermeijer, W. G. H. Han, C. van Els, P. Kaaijk, The Human CD4(+) T Cell Response against Mumps Virus Targets a Broadly Recognized Nucleoprotein Epitope. J Virol 93, (2019).
88. M. May, C. A. Rieder, R. J. Rowe, Emergent lineages of mumps virus suggest the need for a polyvalent vaccine. Int J Infect Dis 66, 1-4 (2018).
89. M. Ramamurthy, P. Rajendiran, N. Saravanan, S. Sankar, S. Gopalan, B. Nandagopal, Identification of immunogenic B-cell epitope peptides of rubella virus E1 glycoprotein towards development of highly specific immunoassays and/or vaccine. Conference Abstract, (2019).
90. U. S. F. Tambunan, F. R. P. Sipahutar, A. A. Parikesit, D. Kerami, Vaccine Design for H5N1 Based on B- and T-cell Epitope Predictions. Bioinform Biol Insights 10, 27-35 (2016).
91. Asante, E A. et. al. “A naturally occurring variant of the human prion protein completely prevents prion disease”. Nature. (2015).
92. Crabtree, G. R. & Schreiber, S. L. Three-part inventions: intracellular signaling and induced proximity. Trends Biochem. Sci. 21, 418-22 (1996).
93. Liu, J. et al. Calcineurin Is a Common Target of A and FKBP-FK506 Complexes. Cell 66, 807-815 (1991).
94. Keith, C. T. et al. A mammalian protein targeted by G1-arresting rapamycin-receptor complex. Nature 369, 756-758 (2003).
95. Spencer, D. M., Wandless, T. J., Schreiber, S. L. S. & Crabtree, G. R. Controlling signal transduction with synthetic ligands. Science 262, 1019-24 (1993).
96. Pruschy, M. N. et al. Mechanistic studies of a signaling pathway activated by the organic dimerizer FK1012. Chem. Biol. 1, 163-172 (1994).
97. Spencer, D. M. et al. Functional analysis of Fas signaling in vivo using synthetic inducers of dimerization. Curr. Biol. 6, 839-847 (1996).
98. Belshaw, P. J., Spencer, D. M., Crabtree, G. R. & Schreiber, S. L. Controlling programmed cell death with a cyclophilin-cyclosporin-based chemical inducer of dimerization. Chem. Biol. 3, 731-738 (1996).
99. Yang, J. X., Symes, K., Mercola, M. & Schreiber, S. L. Small-molecule control of insulin and PDGF receptor signaling and the role of membrane attachment. Curr. Biol. 8, 11-18 (1998).
100. Belshaw, P. J., Ho, S. N., Crabtree, G. R. & Schreiber, S. L. Controlling protein association and subcellular localization with a synthetic ligand that induces heterodimerization of proteins. Proc. Natl. Acad. Sci. 93, 4604-4607 (2002).
101. Stockwell, B. R. & Schreiber, S. L. Probing the role of homomeric and heteromeric receptor interactions in TGF-β signaling using small molecule dimerizers. Curr. Biol. 8, 761-773 (2004).
102. Spencer, D. M., Graef, I., Austin, D. J., Schreiber, S. L. & Crabtree, G. R. A general strategy for producing conditional alleles of Src-like tyrosine kinases. Proc. Natl. Acad. Sci. 92, 9805-9809 (2006).
103. Holsinger, L. J., Spencer, D. M., Austin, D. J., Schreiber, S. L. & Crabtree, G. R. Signal transduction in T lymphocytes using a conditional allele of Sos. Proc. Natd. Acad. Sci. 92, 9810-9814 (2006).
104. Myers, M. G. Insulin Signal Transduction and the IRS Proteins. Annu. Rev. Pharmacol. Toxicol. 36, 615-658 (1996).
105. Watowich, S. S. The erythropoietin receptor: Molecular structure and hematopoietic signaling pathways. J. Investig. Med. 59, 1067-1072 (2011).
106. Blau, C. A., Peterson, K. R., Drachman, J. G. & Spencer, D. M. A proliferation switch for genetically modified cells. Proc. Natl. Acad. Sci. 94, 3076-3081 (2002).
107. Clackson, T. et al. Redesigning an FKBP-ligand interface to generate chemical dimerizers with novel specificity. Proc. Natl. Acad. Sci. 95, 10437-10442 (1998).
108. Diver, S. T. & Schreiber, S. L. Single-step synthesis of cell-permeable protein dimerizers that activate signal transduction and gene expression. J. Am. Chem. Soc. 119, 5106-5109 (1997).
109. Guo, Z. F., Zhang, R. & Liang, F. Sen. Facile functionalization of FK506 for biological studies by the thiol-ene ‘click’ reaction. RSC Adv. 4, 11400-11403 (2014). Robinson, D. R., Wu, Y.-M. & Lin, S.-F. The protein tyrosine kinase family of the human genome. Oncogene 19, 5548-5557 (2000).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A non-naturally occurring polypeptide variant comprising a double-stranded DNA deaminase activity.

2-12. (canceled)

13. A non-naturally occurring polypeptide fragment of a double-stranded DNA deaminase obtained by splitting the deaminase in the deaminase domain at a split site.

14. The non-naturally occurring polypeptide fragment of claim 13, wherein the fragment corresponds to an N-terminal fragment, wherein said fragment comprises an N-terminal portion of a split deaminase domain.

15. The non-naturally occurring polypeptide fragment of claim 13, wherein the fragment corresponds to a C-terminal half fragment, wherein said fragment comprises a C-terminal portion of a split deaminase domain.

16-19. (canceled)

20. The non-naturally occurring polypeptide fragment of claim 14, wherein the N-terminal half fragment comprises an amino acid sequence having at least 90% sequence identity to SEQ ID NO: 349 or 351.

21. The non-naturally occurring polypeptide fragment of claim 15, wherein the C-terminal half fragment comprises an amino acid sequence having at least 90% sequence identity to SEQ ID NO: 350 or 352.

22. A base editor comprising a heterodimer having first and second monomers, said first monomer comprising a first programmable DNA binding protein and an N-terminal fragment of a split double-stranded DNA deaminase, and said second monomer comprising a second programmable DNA binding protein and a C-terminal fragment of a split double-stranded DNA deaminase, wherein dimerization of the first and second monomers reconstitutes the double-stranded DNA deaminase activity.

23-24. (canceled)

25. The base editor of claim 22, wherein the first and/or second programmable DNA binding protein is a nucleic acid programmable DNA binding protein (napDNAbp), a TALE protein, a zinc finger protein, or a mitoTALE protein.

26-36. (canceled)

37. The base editor of claim 22, wherein the N-terminal fragment of the split double-stranded DNA deaminase domain comprises an amino acid sequence having at least 90% sequence identity to SEQ ID NO: 349 or 351; and wherein the C-terminal fragment of the split double-stranded DNA deaminase domain comprises an amino acid sequence having at least 90% sequence identity to SEQ ID NO: 350 or 352.

38-46. (canceled)

47. The base editor of claim 22, wherein the first monomer comprises a linker that joins the first programmable DNA binding protein with the N-terminal fragment of the split double-stranded DNA deaminase, and wherein the second monomer comprises a linker that joins the second programmable DNA binding protein with the C-terminal fragment of the split double-stranded DNA deaminase.

48-51. (canceled)

52. The base editor of claim 22, further comprising one or more uracil glycosylase inhibitor (UGI) domains.

53. (canceled)

54. The base editor of claim 22, further comprising one or more targeting sequences.

55. The base editor of claim 54, wherein the one or more targeting sequences is a nuclear localization sequence (NLS) or a mitochondrial targeting sequence (MTS).

56-57. (canceled)

58. The base editor of claim 5557, wherein the MTS comprises an amino acid sequence selected from the group consisting of: SEQ ID NOs: 13, 14, and 299, or an amino acid sequence having at least 90% sequence identity to SEQ ID NOs: 13, 14, or 299.

59-60. (canceled)

61. The base editor of claim 22, wherein the first and/or second monomers have one of the following structures:
(a) [A]-[programmable DNA binding protein]-[N-terminal or C-terminal fragment of a split double-stranded DNA deaminase]-[B]; or
(b) [A]-[N-terminal or C-terminal fragment of a split double-stranded DNA deaminase]-[programmable DNA binding protein]-[B],
wherein “[A]” and/or “[B]” represent optional one or more additional functional domains; and wherein “]-[” is an optional linker.

62-65. (canceled)

66. The base editor of claim 22, wherein the first monomer comprises one of the following structures: [SOD2]-[UGI]₁₋₂-[mitoTALE]-[DddAₜₒₓ-N of SEQ ID NO: 349 or DddAₜₒₓ-C of SEQ ID NO: 350]-[UGI]₁₋₂ or [COX8A]-[UGI]₁₋₂-[mitoTALE]-[DddAₜₒₓ-N of SEQ ID NO: 351 or DddAₜₒₓ-C of SEQ ID NO: 3521-[UGI]₁₋₂; and
wherein the second monomer comprises one of the following structures: [SOD21-[UGI]₁₋₂-[mitoTALE]-[DddAₜₒₓ-N of SEQ ID NO: 349 or DddAₜₒₓ-C of SEQ ID NO: 350]-[UGI]₁₋₂ or [COX8A]-[UGI]₁₋₂-[mitoTALE]-[DddAₜₒₓ-N of SEQ ID NO: 351 or DddAₜₒₓ-C of SEQ ID NO: 352]-[UGI]₁₋₂.

67. The base editor of claim 66, wherein the first monomer comprises an amino acid sequence having at least 90% sequence identity to SEQ ID NO: 360 or 361, and wherein the second monomer comprises an amino acid sequence having at least 90% sequence identity to SEQ ID NO: 360 or 361.

68-73. (canceled)

74. The base editor of claim 22, wherein the first and second monomers bind to first and second nucleotide sequences, respectively, on either side of a target site; and wherein the target site comprises a target base that becomes deaminated by the base editor.

75-84. (canceled)

85. An isolated nucleic acid encoding the first monomer and/or the second monomer of the base editor of claim 22.

86-88. (canceled)

89. A vector comprising the isolated nucleic acid of claim 85.

90. A cell comprising a vector of claim 89.

91. A method of editing a target nucleotide sequence at a target site, comprising contacting a target nucleotide sequence with a base editor of claim 22, wherein the first monomer targets a first nucleotide sequence flanking a target site, and the second monomer targets a second nucleotide sequence flanking the target site, thereby inducing deamination of a target base at the target site.

92-110. (canceled)

111. A method of delivering a base editor of claim 22 to a cell comprising transforming a cell with one or more vectors encoding the first and second monomers of the base editor, wherein once in the cell the first and second monomers are expressed and dimerize, thereby forming a base editor in the cell.

112-114. (canceled)

115. A method of delivering a base editor of claim 22 to a mitochondria comprising transforming a cell with one or more vectors encoding the first and second monomers of the base editor, wherein once in the cell the first and second monomers are expressed, transported to the mitochondria, and dimerize therein, thereby forming a base editor in the mitochondria, wherein the first and second programmable DNA binding proteins of the base editor are mitoTALE domains.

116-121. (canceled)
